(19) United States
(12) Patent Application Publication
(10) Pub. No.: US 2026/0241012 A1
(43) Pub. Date: Aug. 20, 2026

(54) 1,8-NAPHTHYRIDIN-2-ONE HETEROBIFUNCTIONAL BCL6 DEGRADERS

(71) Applicant: Treeline Biosciences, Inc., Watertown, MA (US)

(72) Inventors: Kevin M. Oberg, San Diego, CA (US); Joshua D. Hansen, San Diego, CA (US); Matthew D. Correa, San Diego, CA (US); Jeffrey A. Engelman, Brookline, MA (US); Joel D. Leverson, San Diego, CA (US); Tami J. Marrone, Carlsbad, CA (US); Matthew H. McNeill, San Clemente, CA (US); Mark A. Nagy, Encinitas, CA (US); Kristin D. Schleicher, San Diego, CA (US); Andrew Valiere, EI Cajon, CA (US); Ming Yan, San Diego, CA (US)

(21) Appl. No.: 18/874,504

(22) PCT Filed: Jun. 5, 2023
(86) PCT No.: PCT/US2023/067918

§ 371 Date: Dec. 12, 2024

Related U.S. Application Data
(60) Provisional application No. 63/351,697, filed on Jun. 13, 2022.
(60) Provisional application No. 63/351,715, filed on Jun. 13, 2022.
(60) Provisional application No. 63/395,630, filed on Aug. 5, 2022.
(60) Provisional application No. 63/395,638, filed on Aug. 5, 2022.
(60) Provisional application No. 63/407,006, filed on Sep. 15, 2022.
(60) Provisional application No. 63/407,012, filed on Sep. 15, 2022.
(60) Provisional application No. 63/420,421, filed on Oct. 28, 2022.
(60) Provisional application No. 63/436,009, filed on Dec. 29, 2022.
(60) Provisional application No. 63/444,801, filed on Feb. 10, 2023.
(60) Provisional application No. 63/497,054, filed on Apr. 19, 2023.
(60) Provisional application No. 63/501,077, filed on May 9, 2023.

(51) Int. Cl. A61K 47/55; A61K 47/54

(52) U.S. Cl. CPC A61K 47/55; A61K 47/545

(57) ABSTRACT

This disclosure provides compounds of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, that induce degradation of a BCL6 protein. These compounds are useful, for example, for treating a cancer in a subject (e.g., a human). This disclosure also provides compositions containing the same as well as methods of using and making the same.

### CROSS-REFERENCE TO RELATED APPLICATIONS

This application claims priority to U.S. Provisional Application Ser. No. 63/351,715, filed Jun. 13, 2022; 63/351,697, filed Jun. 13, 2022; 63/395,630, filed Aug. 5, 2022; 63/395,638, filed Aug. 5, 2022; 63/407,006, filed Sep. 15, 2022; 63/407,012, filed Sep. 15, 2022; 63/420,421, filed Oct. 28, 2022; 63/436,009, filed Dec. 29, 2022; 63/444,801, filed Feb. 10, 2023; 63/497,054, filed Apr. 19, 2023; and 63/501,077, filed May 9, 2023; each of which is hereby incorporated by reference in its entirety.

### DESCRIPTION OF THE TEXT FILE SUBMITTED ELECTRONICALLY

This application contains a Sequence Listing which has been submitted electronically in XML format. The Sequence Listing XML is incorporated herein by reference. Said XML file, created on May 26, 2023, is named TLS-037WO_SL.xml and is 2,663 bytes in size.

### TECHNICAL FIELD

This disclosure provides compounds of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or pharmaceutically acceptable salts thereof, that induce degradation of a BCL6 protein. These compounds are useful, for example, for treating cancer in a subject (e.g., a human). This disclosure also provides compositions containing the same as well as methods of using and making the same.

### BACKGROUND

B-cell lymphoma 6 (BCL6) protein is a transcriptional repressor involved in the formation and maintenance of germinal centers (GCs) within lymphoid follicles. It controls the functions of the GC and coordinates the activities of signaling mediators in the maturation of GC B cells. There are over 1000 known or putative BCL6 target genes, including MYC, BCL2, genes related to DNA damage response (e.g., ATR, TP53), and cell cycle checkpoint control (e.g., CDKN1A, CDKN1B). BCL6 is expressed in the dark zone cells of GCs, where somatic hypermutation is allowed to occur to generate high-affinity B-cell receptors. Overexpression or loss of control of BCL6, for example by translocation, can permit maintenance of the pro-hypermutation functions and abrogation of the antitumor functions of BCL6.

### SUMMARY

Provided herein are compounds of Formula (I):

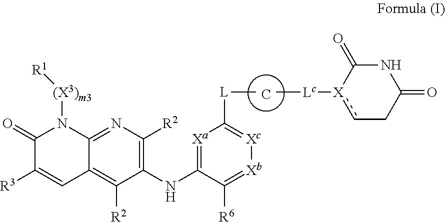

or pharmaceutically acceptable salts thereof, wherein:
R¹ is selected from the group consisting of: H, halo, cyano, and R^{b1};
m3 is 0, 1, 2, or 3;
each X³ is independently selected from the group consisting of: —O—, —NR^{f}—, —C(═O)—, and C₁₋₃ alkylene optionally substituted with 1-3 R^{c};
provided that the N—(X³)ₘ₃—R¹ moiety does not contain any O—O, N—O, N—N, O-halo, or N-halo bonds;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e},
R³ is selected from the group consisting of:
A¹-C(R⁴R⁴)-A² and —CH═CH-A², wherein:
A¹ is —O— or —S—;
each R⁴ is independently selected from the group consisting of: H, C₁₋₆ alkyl, and C₁₋₆ haloalkyl; or
the pair of R⁴ taken together with the carbon atom to which each is attached form a C₃₋₆ cycloalkyl ring or a 4-8 membered heterocyclyl ring, wherein the C₃₋₆ cycloalkyl ring or 4-8 membered heterocyclyl ring is optionally substituted with 1-3 R⁸;
A² is selected from the group consisting of: —C(O)OH, —C(O)NH₂, —C(O)R³⁴, —C(O)OR^{3A}, —C(O)NR^{3A}R^{f}, —S(O)₁₋₂ (C₁₋₆ alkyl), —P(O)—(C₁₋₆ alkyl)₂, and —C(═NH)NH₂, wherein:
R^{3A} is selected from the group consisting of: C₁₋₆ alkyl, C₃₋₆ alkenyl, C₃₋₆ alkynyl, C₃₋₆ cycloalkyl, and 3-8 membered heterocyclyl, each of which is optionally substituted with 1-6 substituents independently selected from the group consisting of R^{a} and —(C₀₋₃ alkylene)-R^{b1};
X^{a} and X^{c} are independently selected from the group consisting of: N, CH, and CF, provided that one or both of X^{a} and X^{c} is N;
X^{b} is selected from the group consisting of N and CR^{x1};
R⁶ and R^{x1} are each independently selected from the group consisting of: H, halo, C₁₋₂ alkyl, C₁₋₂ haloalkyl, C₁₋₂ alkoxy, CN, and —C═CH;
L is -(L^{A})ₙ₁-, wherein L^{A} and n1 are defined according to (AA) or (BB):
   (AA)
n1 is an integer from 1 to 15; and
each L^{A} is independently selected from the group consisting of: L^{A1}, L^{A3}, and L^{A4}, provided that 1-3 occurrences of L^{A} is L^{A4};
   (BB)
n1 is an integer from 0 to 20; and
each L^{A} is independently selected from the group consisting of: L^{A1} and L^{A3}, each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, O—O, N—N, N—S(O)₀, or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, S(O)₀₋₂(C₁₋₆ alkyl), S(O)₀₋₂ (C₁₋₆ haloalkyl), S(O)₁₋₂N(R^{f})₂, —R^{b}, and C₁₋₆ alkyl optionally substituted with 1-6 R^{c};
Ring C is selected from the group consisting of:

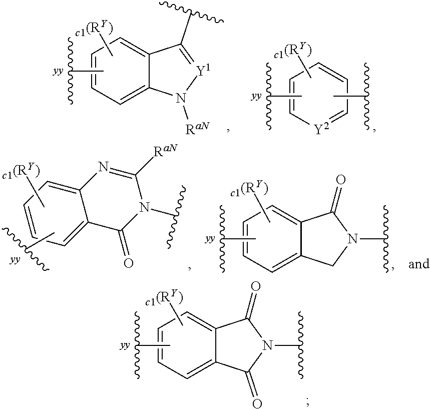

c1 is 0, 1, 2, or 3;
each R^{Y} is independently selected from the group consisting of: R^{a} and R^{b};
R^{aN} is H or C₁₋₆ alkyl optionally substituted with 1-3 R^{c};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —C(═O)—, —N(R^{d})—, and O, provided that when X is N, then L^{C} is other than O; and further provided that when Ring C is attached to -L^{C}- via a ring nitrogen, then X is CH, and LC is a bond;
each R^{a} is independently selected from the group consisting of:
(a) halo;
(b) cyano;
(c) —OH;
(d) oxo;
(e) C₁₋₆ alkoxy optionally substituted with 1-6 R^{c};
(f) —NR^{d}R^{e};
(g) C(═O)C₁₋₆ alkyl optionally substituted with 1-6 R^{c};
(h) C(═O)OH;
(i) C(═O)OC₁₋₆ alkyl;
(j) C(═O)OC₁₋₆ haloalkyl;
(k) C(═O)N(R^{f})₂;
(l) S(O)₀₋₂ (C₁₋₆ alkyl);
(m) S(O)₀₋₂ (C₁₋₆ haloalkyl);
(n) S(O)₁₋₂N(R^{f})₂; and
(o) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂ (C₁₋₆ alkyl), S(O)₀₋₂ (C₁₋₆ haloalkyl), and S(O)₁₋₂N(R^{f})₂;
each R^{d} and R^{e} is independently selected from the group consisting of: H, C(—O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, cyano, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H) (C₁₋₃ alkyl), and —(C₀₋₃ alkylene)-N(C₁₋₃ alkyl)₂.

Also provided herein are pharmaceutical compositions comprising a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, and a pharmaceutically acceptable carrier.

Provided herein are methods for treating cancer in a subject in need thereof, the methods comprising administering to the subject a therapeutically effective amount of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition as provided herein.

Also provided herein are BCL6 proteins non-covalently bound with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof.

Also provided herein are ternary complexes comprising a BCL6 protein, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, and a CRBN protein, or a portion thereof.

To facilitate understanding of the disclosure set forth herein, a number of additional terms are provided. Generally, the nomenclature used herein and the laboratory procedures in organic chemistry, medicinal chemistry, and pharmacology described are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties. In the case of conflict between the present disclosure and any content incorporated by reference, the present disclosure controls.

The details of one or more embodiments of the invention are set forth in the accompanying drawings and the description below. Other features and advantages of the invention will be apparent from the description and drawings, and from the claims.

### DETAILED DESCRIPTION

This disclosure provides compounds of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or pharmaceutically acceptable salts thereof, that induce degradation of a BCL6 protein. These compounds are useful, e.g., for treating a cancer. This disclosure also provides compositions containing the compounds provided herein as well as methods of using and making the same.

Upon antigen challenge, germinal centers (GCs) are formed in lymphoid follicles, and B-cells in the dark zone of GCs undergo rapid proliferation and somatic hypermutation, both of their immunoglobin variable genes to generate high-affinity B-cell receptors, as well as of other genes including BCL6. BCL6 is often considered to be a ‘master regulator’ of the GC reaction. In some cancers, BCL6 can be mutated, translocated, and/or BCL6 expression can be upregulated. See, e.g., Leeman-Neill and Bhagat, Expert Opinion on Therapeutic Targets 22.2 (2018): 143-152; Mlynarczyk and Melnick. Immunological Reviews 288.1 (2019): 214-239.

The BCL6 protein has multiple domains, including a BTB domain, an RD2 domain, and a DNA binding domain. The N-terminal BTB domain is the site of homodimerization of BCL6, and the interface of the monomers forms the “lateral groove”, which is a binding site for endogenous co-repressors of BCL6, such as SMRT, NCOR, and BCOR. See, e.g., Cardenas, Mariano G., et al. Clinical Cancer Research 23.4 (2017): 885-893.

Compounds that induce degradation of a target protein are sometimes referred to as heterobifunctional compounds, PROTACs, or degraders. Such compounds generally include a moiety that binds to the target protein and a moiety that binds to a ubiquitin E3 ligase (sometimes referred to as an E3 ligase or simply an E3), these two moieties being optionally separated by a linker. To induce degradation, heterobifunctional compounds are believed to induce formation of a ternary complex between the target protein, the compound, and an E3 ligase. Formation of the ternary complex is then followed by ubiquitination of the target protein and degradation of the ubiquitinated target protein by a proteosome. Several E3 ligases have been used as the partner E3 ligase for heterobifunctional degraders. Herein, the cereblon (CRBN) E3 ligase (also referred to herein as a CRBN protein) is used.

A degradation approach for a target protein can have potential advantages compared to, e.g., small molecule inhibition of the target protein. One potential advantage is that the duration of effect of a heterobifunctional compound is generally based on the resynthesis rate of the target protein. Another potential advantage is that many heterobifunctional compounds are believed to be released from the ubiquitinated target protein-E3 ligase complex and made available for formation of further ternary complexes; this is sometimes referred to as “catalytic” turnover of the heterobifunctional compound. Degradation of a target protein can also be advantageous over small molecule inhibition in some cases, as degradation can impair a scaffolding function of a target protein, whereas a small molecule might not. It is also generally believed that for formation of a ternary complex, high affinity to the target protein is not always required.

Heterobifunctional compounds are further described in, for example, International Publication Nos. WO 2021/077010 and WO 2022/221673; McCoull, William, et al., ACS Chemical Biology 13.11 (2018): 3131-3141; Chamberlain and Hamann, Nature Chemical Biology 15.10 (2019): 937-944; Li and Song, Journal of Hematology & Oncology 13 (2020): 1-14; Wu, et al. Nature Structural & Molecular Biology 27.7 (2020): 605-614; Dong, et al., Journal of Medicinal Chemistry 64.15 (2021): 10606-10620; Yang, et al., Targeted Oncology 16.1 (2021): 1-12.

### Compound Embodiments

Provided herein are compounds of Formula (I):

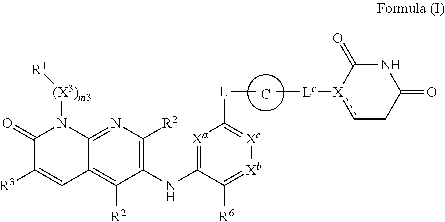

or pharmaceutically acceptable salts thereof, wherein:
R¹ is selected from the group consisting of: H, halo, cyano, and R^{b1};
m3 is 0, 1, 2, or 3;
each X³ is independently selected from the group consisting of: —O—, —NR^{f}—, —C(═O)—, and C₁₋₃ alkylene optionally substituted with 1-3 R^{c};
provided that the N—(X³)ₘ₃—R¹ moiety does not contain any O—O, N—O, N—N, O-halo, or N-halo bonds;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e};
R³ is selected from the group consisting of:
A¹-C(R⁴R⁴)-A² and —CH—CH-A², wherein:
A¹ is —O— or —S—;
each R⁴ is independently selected from the group consisting of: H, C₁₋₆ alkyl, and C₁₋₆ haloalkyl; or
the pair of R⁴ taken together with the carbon atom to which each is attached form a C₃₋₆ cycloalkyl ring or a 4-8 membered heterocyclyl ring, wherein the C₃₋₆ cycloalkyl ring or 4-8 membered heterocyclyl ring is optionally substituted with 1-3 R^{g};
A² is selected from the group consisting of: —C(O)OH, —C(O)NH₂, —C(O)R^{3A}, —C(O)OR^{3A}, —C(O)NR^{3A}R^{f}, —S(O)₁₋₂ (C₁₋₆ alkyl), —P(O)—(C₁₋₆ alkyl)₂, and —C(═NH)NH₂, wherein:
R^{3A} is selected from the group consisting of: C₁₋₆ alkyl, C₃₋₆ alkenyl, C₃₋₆ alkynyl, C₃₋₆ cycloalkyl, and 3-8 membered heterocyclyl, each of which is optionally substituted with 1-6 substituents independently selected from the group consisting of R^{a} and —(C₀₋₃ alkylene)-R^{b1};
X^{a} and X^{c} are independently selected from the group consisting of: N, CH, and CF, provided that one or both of X^{a} and X^{c} is N;
X^{b} is selected from the group consisting of N and CR^{x1};
R⁶ and R^{x1} are each independently selected from the group consisting of: H, halo, C₁₋₂ alkyl, C₁₋₂ haloalkyl, C₁₋₂ alkoxy, CN, and —C═CH;
L is -(L^{A})ₙ₁-, wherein L^{A} and n1 are defined according to (AA) or (BB):
   (AA)
n1 is an integer from 1 to 15; and
each L^{A} is independently selected from the group consisting of: L^{A1}, L^{A3}, and L^{A4}, provided that 1-3 occurrences of L^{A} is L^{A4}.
   (BB)
n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, O—O, N—N, N—S(O) o, or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, S(O)₀₋₂ (C₁₋₆ alkyl), S(O)₀₋₂ (C₁₋₆ haloalkyl), S(O)₁₋₂N(R^{f})₂, —R^{b}, and C₁₋₆ alkyl optionally substituted with 1-6 R^{e};
Ring C is selected from the group consisting of:

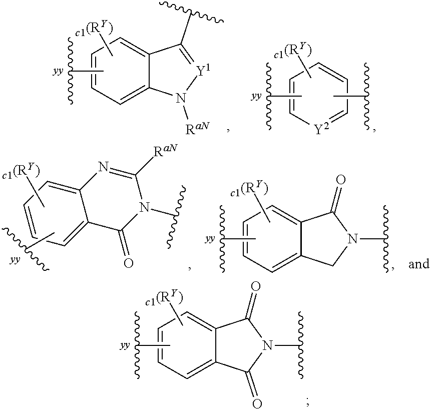

c1 is 0, 1, 2, or 3;
each R^{Y} is independently selected from the group consisting of: R^{a} and R^{b};
R^{aN} is H or C₁₋₆ alkyl optionally substituted with 1-3 R^{c};
Y¹ and Y² are independently N, CH, or CRY;
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —C(═O)—, —N(R^{d})—, and O, provided that when X is N, then L^{C} is other than O; and
further provided that when Ring Cis attached to -L^{C}- via a ring nitrogen, then X is CH, and L^{C} is a bond;
each R^{a} is independently selected from the group consisting of:
(a) halo;
(b) cyano;
(c) —OH;
(d) oxo;
(e) C₁₋₆ alkoxy optionally substituted with 1-6 R^{c};
(f) —NR^{d}R^{e};
(g) C(═O)C₁₋₆ alkyl optionally substituted with 1-6 R^{c};
(h) C(═O)OH;
(i) C(═O)OC₁₋₆ alkyl;
(j) C(═O)OC₁₋₆ haloalkyl;
(k) C(═O)N(R^{f})₂;
(l) S(O)₀₋₂ (C₁₋₆ alkyl);
(m) S(O)₀₋₂ (C₁₋₆ haloalkyl);
(n) S(O)₁₋₂N(R^{f})₂; and
(o) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{e} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═)OC₁₋₆ alkyl, C(═O)OC₁₋₆ haloalkyl, C(═O)OH, C(═O)N(R^{f})₂, S(O)₀₋₂ (C₁₋₆ alkyl), S(O)₀₋₂ (C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂ (C₁₋₆ haloalkyl), S(O)₁₋₂N(R^{f})₂, and C₁₋₆ alkyl optionally substituted with 1-3 R^{h},
each R^{f} is independently selected from the group consisting of: H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, cyano, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H) (C₁₋₃ alkyl), and —(C₀₋₃ alkylene)-N(C₁₋₃ alkyl)₂.

In some embodiments, R³ is -A¹-C(R⁴R⁴)-A².

In some embodiments, A¹ is —O—.

In some embodiments, each R⁴ is H. In some embodiments, one R⁴ is C₁₋₃ alkyl (e.g., methyl); and the other R⁴ is H.

In some embodiments, R³ is —CH═CH-A².

In some embodiments, A² is —C(O)NH₂ or —C(O)NR^{3A}R^{f}. In some embodiments, R^{3A} is C₁₋₃ alkyl optionally substituted with 1-6 R^{c}. In some embodiments, A² is —C(O)NH₂, —C(O)NHMe, or —C(O)NMe₂. For example, A² can be —C(O)NHMe.

In some embodiments, R³ is -A¹-C(R⁴R⁴)-A²; A¹ is O; each R⁴ is H; and A² is —C(O)NH₂ or —C(O)NR^{3A}R^{f}, wherein R^{3A} is C₁₋₃ alkyl optionally substituted with 1-6 R^{c}. In some embodiments, A² is —C(O)NH₂, —C(O)NHMe, or —C(O)NMe₂.

In some embodiments, R³ is

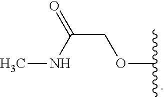 [SMILES: *OCC(=O)NC]

In some embodiments, the compounds of Formula (I) are compounds of Formula (I-1):

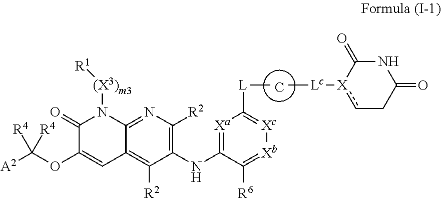

or pharmaceutically acceptable salts thereof, wherein:
A² is —C(O)NH₂ or —C(O)NR^{3A}R^{f}; and
each R⁴ is independently H or C₁₋₃ alkyl.

In some embodiments of Formula (I-1), each R⁴ is H.

In some embodiments of Formula (I-1), A² is —C(O)NH₂, —C(O)NHMe, or —C(O)NMe₂. For example, A² can be —C(O)NHMe.

In some embodiments of Formula (I-1), each R⁴ is H; and A² is —C(O)NH₂, —C(O)NHMe, or —C(O)NMe₂. For example, A² can be —C(O)NHMe.

In some embodiments, Xa is N; X^{c} is N; and X^{b} is CR^{x1}. For example, Xa can be N; X^{c} can be N; and X^{b} can be CH.

In some embodiments, X^{a} is CH; X^{c} is N; and X^{b} is CR^{x1} (e.g., CH). For example, X^{a} can be CH; X^{c} can be N; and X^{b} can be CH.

In some embodiments, R⁶ is halo (e.g., —F, —C₁, —Br) or CN. For example, R⁶ can be —Cl, —F, or CN.

In some embodiments, R⁶ is —Cl or —F.

In some embodiments, each R² is H.

In some embodiments, m3 is 0.

In some embodiments, m3 is 1; and X³ is C₁₋₃ alkylene (e.g., methylene, ethylene, or isopropylene).

In some embodiments, R¹ is H.

In some embodiments, R¹ is R^{b1}. In some embodiments, R¹ is C₃₋₆ cycloalkyl or 4-6 membered heterocyclyl, each of which is optionally substituted with 1-3 R⁸. For example, R¹ can be selected from the group consisting of:

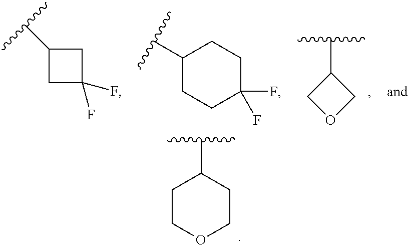

In some embodiments, m3 is 1; X³ is methylene, ethylene, or isopropylene; and R¹ is H.

In some embodiments, R³ is -A¹-C(R⁴R⁴)-A², wherein A¹ is O; each R⁴ is H; and A² is —C(O)NH₂ or —C(O)NR^{3A}R^{f}, wherein R^{3A} is C₁₋₃ alkyl optionally substituted with 1-6 R^{c};
each R² is H; X^{a} is N or CH; X^{c} is N; X^{b} is CH; and R⁶ is —F or —C₁. In some embodiments, m3 is 1; X³ is methylene, ethylene, or isopropylene; and R¹ is H. In some embodiments, X^{a} is N.

In some embodiments, the

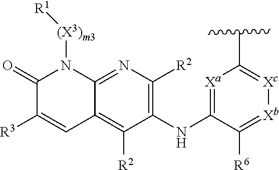

moiety is

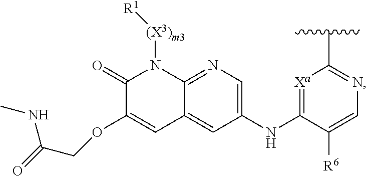

wherein:
X^{a} is N or CH; R⁶ is —F or —Cl;
m3 is 1; X³ is C₁₋₃ alkylene; and
R¹ is H. For example, X³ can be isopropylene.

In some embodiments, Ring C is

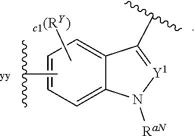

In some embodiments, Ring C is

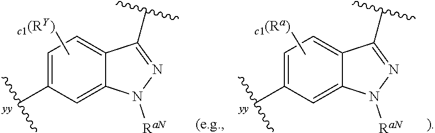

In some embodiments, Ring C is

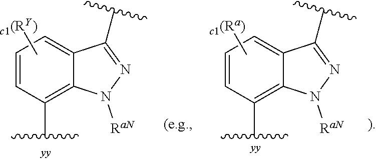

In some embodiments, c1 is 0. In some embodiments, c1 is 1; and R^{Y} is halo (e.g., —F). In some embodiments, R^{aN} is C₁₋₃ alkyl (e.g., methyl).

In some embodiments, Ring C is

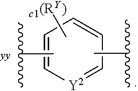

In some embodiments, c1 is 0. In some embodiments, c1 is 1; and R^{Y} is halo (e.g., —F).

In some embodiments, Ring C is selected from the group consisting of:

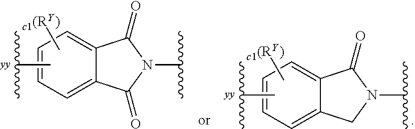

In some embodiments, c1 is 0. In some embodiments, c1 is 1; and R^{Y} is halo (e.g., —F).

In some embodiments, c1 is 0. In some embodiments, c1 is 1; and R^{Y} is halo (e.g., —F). In some embodiments, R^{aN} is C₁₋₃ alkyl (e.g., methyl).

In some embodiments, X is CH. In some embodiments, X is N.

In some embodiments, L^{C} is a bond.

In some embodiments, the

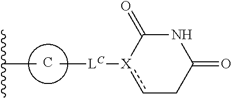

moiety is selected from the group consisting of:

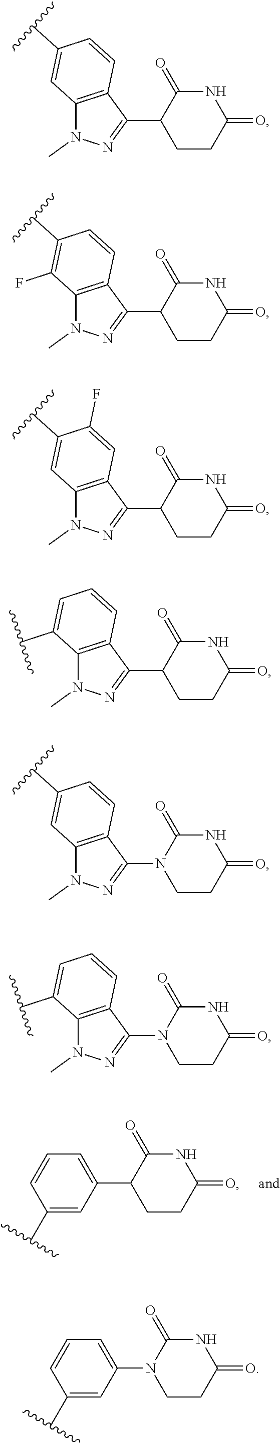

In some embodiments, the

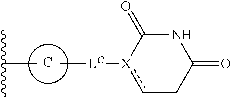

moiety is selected from the group consisting of:

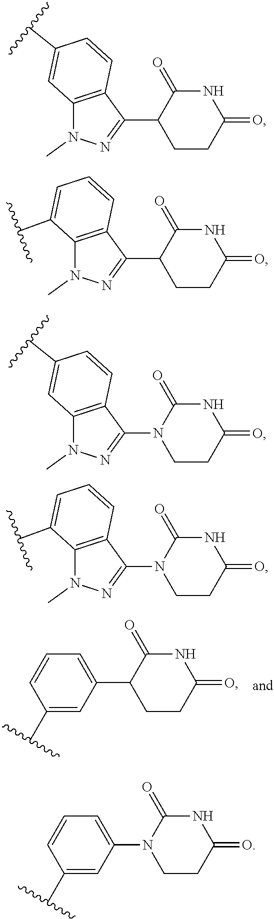

In some embodiments, the

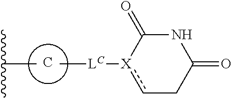

moiety is selected from the group consisting of:

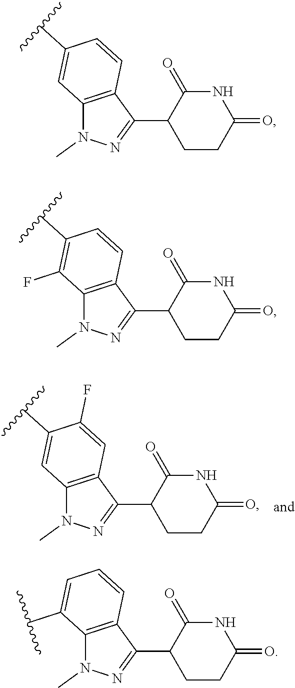

In some embodiments, the

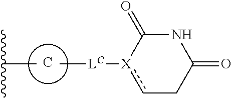

moiety is selected from the group consisting of:

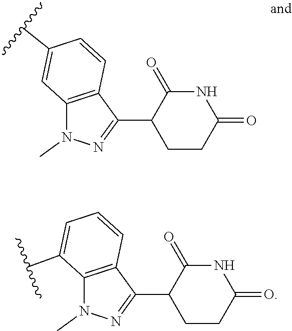 [SMILES: *c1ccc2c(C3CCC(=O)NC3=O)nn(C)c2c1 ; *c1cccc2c(C3CCC(=O)NC3=O)nn(C)c12]

In some embodiments, the

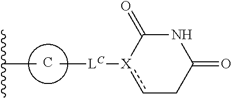

moiety is selected from the group consisting of:

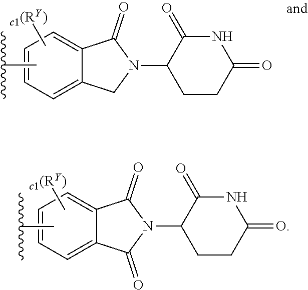

In some embodiments, the

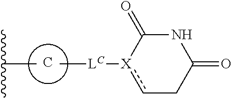

moiety is

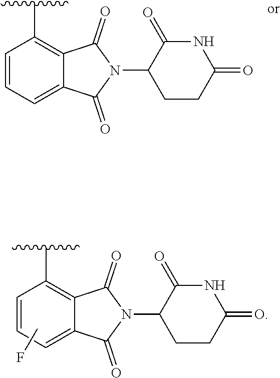

In some embodiments, L is -(L^{A})ₙ₁-, wherein L^{A} and n1 are defined according to (AA). In some embodiments, n1 is an integer from 1 to 5. In some embodiments, n1 is an integer from 2 to 5 (e.g., 2 or 3).

In some embodiments, L is selected from the group consisting of:

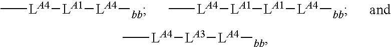

wherein bb represents the point of attachment to Ring C.

In some embodiments, each L^{A4} is independently a C₃₋₁₀ cycloalkylene or 4-12 membered heterocyclylene, each of which is optionally substituted with 1-6 R^{a}.

In some embodiments, each L^{A4} is independently a 4-12 membered heterocyclylene optionally substituted with 1-6 R^{a}

In some embodiments, each L^{A4} is independently a 4-10 membered heterocyclylene optionally substituted with 1-3 R^{a}.

In some embodiments, each L^{A4} is independently a monocyclic 4-6 membered nitrogen-containing heterocyclylene optionally substituted with 1-3 R^{a}.

In some embodiments, one L^{A4} is a monocyclic 4-6 membered monocyclic nitrogen-containing heterocyclylene optionally substituted with 1-3 R^{a}; and the other L^{A4} is a bicyclic 6-12 membered nitrogen-containing heterocyclylene optionally substituted with 1-3 R^{a}. In some embodiments, the other L^{A4} is a spirocyclic bicyclic 6-10 membered nitrogen-containing heterocyclylene optionally substituted with 1-3 R^{a}. In some embodiments, the other L^{A4} is a bridged bicyclic 6-10 membered nitrogen-containing heterocyclylene optionally substituted with 1-3 R^{a}.

In some embodiments, each R^{a} present on L^{A4} is independently selected from the group consisting of: —F; CN; C₁₋₃ alkoxy; OH; C₁₋₃ alkyl optionally substituted with 1-3 F; and C₁₋₃ alkyl substituted with —OH.

In some embodiments, each R^{a} present on L^{A4} is independently selected from the group consisting of: —F, CN, C₁₋₃ alkoxy, OH, and C₁₋₃ alkyl optionally substituted with 1-3 F.

In some embodiments, each R^{a} present on L^{A4} is independently selected from the group consisting of: F, methyl, CF₃, CHF₂, and CH₂F.

In some embodiments, each L^{A4} contains 1-2 ring nitrogen atoms and no additional ring heteroatoms.

In some embodiments, L is -L^{A4}-L^{A1}-L^{A4}-_{bb}, wherein bb represents the point of attachment to Ring C. In some embodiments, L^{A1} is —CH₂—. In some embodiments, L^{A1} is —CHMe-. In some embodiments, L^{A1} is —C(Me)₂-.

In some embodiments, L is -L^{A4}-L^{A3}-L^{A4}-_{bb}, wherein bb represents the point of attachment to Ring C. In some embodiments, L^{A3} is —O—, —NH—, or —N(C₁₋₃ alkyl)-.

In some embodiments, L is -L^{A4}-L^{A1}_L^{A1}-L^{A4}-_{bb}, wherein bb represents the point of attachment to Ring C. In some embodiments, each L^{A1} is CH₂.

In some embodiments, L is -L^{A4}-L^{A1}-(L^{A3})_{1-2-bb}, wherein bb represents the point of attachment to Ring C. In some embodiments, L is -L^{A4}-L^{A1}-L^{A3}-_{bb}, wherein bb represents the point of attachment to Ring C. In some embodiments, L^{A1} is —CH₂—. In some embodiments, L^{A3} is NH or N(C₁₋₃ alkyl) (e.g., NH). In some embodiments, L is -L^{A4}-CH₂—NH—_{bb}, wherein bb represents the point of attachment to Ring C. In some embodiments, L^{A4} is a 4-10 membered heterocyclylene optionally substituted with 1-3 R^{a}. In some embodiments, L^{A4} is a monocyclic 4-6 membered nitrogen-containing heterocyclylene optionally substituted with 1-3 R^{a}.

In some embodiments, L is -L^{A4}-L^{A3}-_{bb}, wherein bb represents the point of attachment to Ring C. In some embodiments, L^{A3} is NH. In some embodiments, L^{A3} is —O—. In some embodiments, L^{A4} is a monocyclic 4-8 (e.g., 4-6) membered nitrogen-containing heterocyclylene optionally substituted with 1-3 R^{a}. In some embodiments, L^{A4} is a spirocyclic bicyclic 6-12 (e.g., 8-12) membered nitrogen-containing heterocyclylene optionally substituted with 1-3 R^{a}.

In some embodiments, L is -L^{A4}-L^{A4}_L^{A3}-L^{A4}-_{bb} or -L^{A4}_L^{A3}-L^{A4}-L^{A4}-_{bb}, wherein bb represents the point of attachment to Ring C.

In some embodiments, L is -L^{A4}-(C₃₋₆ cycloalkylene)-O-L^{A4}-_{bb} or -L^{A4}-O—(C₃₋₆ cycloalkylene)-L^{A4}-_{bb}, wherein the C₃₋₆ cycloalkylene is optionally substituted with 1-2 R^{a}; and each L^{A4} is independently a 4-12 membered heterocyclylene (e.g., 5-6 membered (e.g., 6-membered)) optionally substituted with 1-2 R^{a}.

In some embodiments, L is

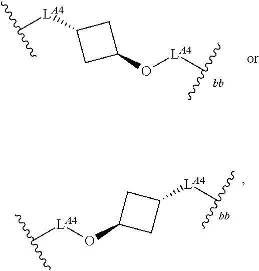

wherein each L^{A4} is a monocyclic 4-6 membered heterocyclylene optionally substituted with 1-2 R^{a}.

In some embodiments, L is selected from the group consisting of the moieties delineated in Table L, wherein bb represents the point of attachment to Ring C.

TABLE L
Examples when L is -L-^{A4}-L^{A1}-L^{A4}-_{bb}

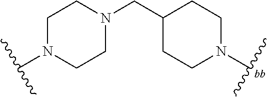 [SMILES: *N1CCC(CN2CCN(*)CC2)CC1]

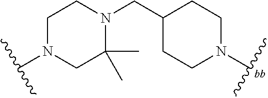 [SMILES: *N1CCC(CN2CCN(*)CC2(C)C)CC1]

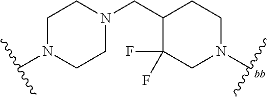 [SMILES: *N1CCN(CC2CCN(*)CC2(F)F)CC1]

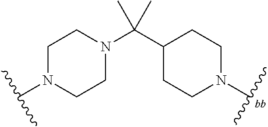 [SMILES: *N1CCC(C(C)(C)N2CCN(*)CC2)CC1]

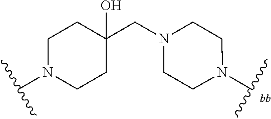 [SMILES: *N1CCN(CC2(O)CCN(*)CC2)CC1]

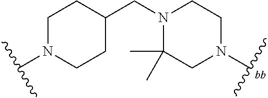 [SMILES: *N1CCC(CN2CCN(*)CC2(C)C)CC1]

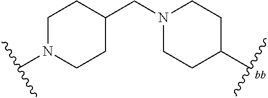 [SMILES: *C1CCN(CC2CCN(*)CC2)CC1]

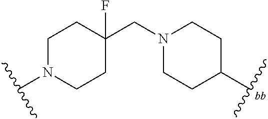 [SMILES: *C1CCN(CC2(F)CCN(*)CC2)CC1]

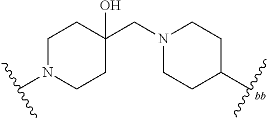 [SMILES: *C1CCN(CC2(O)CCN(*)CC2)CC1]

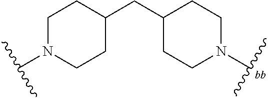 [SMILES: *N1CCC(CC2CCN(*)CC2)CC1]

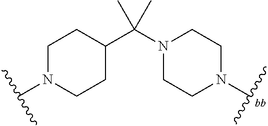 [SMILES: *N1CCC(C(C)(C)N2CCN(*)CC2)CC1]

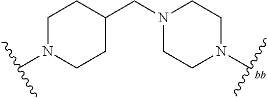 [SMILES: *N1CCC(CN2CCN(*)CC2)CC1]

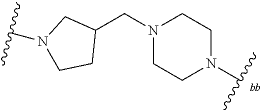 [SMILES: *N1CCN(CC2CCN(*)C2)CC1]

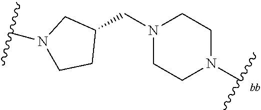 [SMILES: *N1CCN(C[C@@H]2CCN(*)C2)CC1]

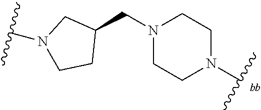 [SMILES: *N1CCN(C[C@H]2CCN(*)C2)CC1]

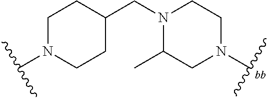 [SMILES: *N1CCC(CN2CCN(*)CC2C)CC1]

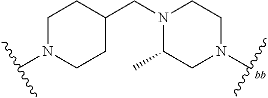 [SMILES: *N1CCC(CN2CCN(*)C[C@@H]2C)CC1]

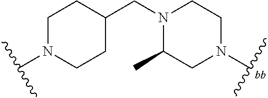 [SMILES: *N1CCC(CN2CCN(*)C[C@H]2C)CC1]

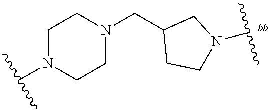 [SMILES: *N1CCN(CC2CCN(*)C2)CC1]

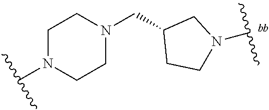 [SMILES: *N1CCN(C[C@H]2CCN(*)C2)CC1]

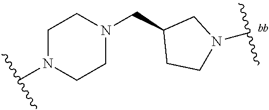 [SMILES: *N1CCN(C[C@@H]2CCN(*)C2)CC1]

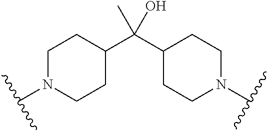 [SMILES: *N1CCC(C(C)(O)C2CCN(*)CC2)CC1]

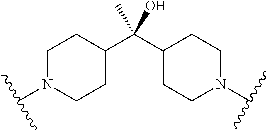

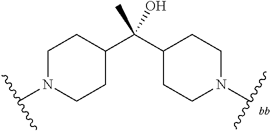

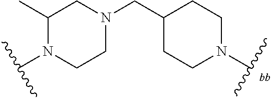 [SMILES: *N1CCC(CN2CCN(*)C(C)C2)CC1]

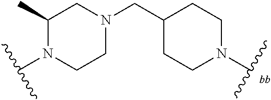 [SMILES: *N1CCC(CN2CCN(*)[C@@H](C)C2)CC1]

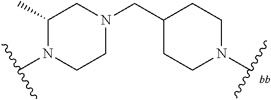 [SMILES: *N1CCC(CN2CCN(*)[C@H](C)C2)CC1]

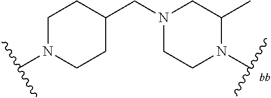 [SMILES: *N1CCC(CN2CCN(*)C(C)C2)CC1]

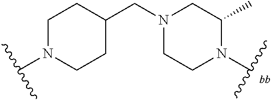 [SMILES: *N1CCC(CN2CCN(*)[C@@H](C)C2)CC1]

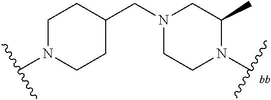 [SMILES: *N1CCC(CN2CCN(*)[C@H](C)C2)CC1]

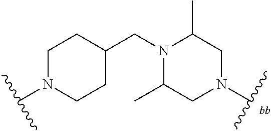 [SMILES: *N1CCC(CN2C(C)CN(*)CC2C)CC1]

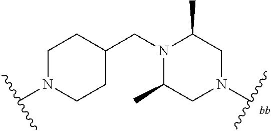 [SMILES: *N1CCC(CN2[C@H](C)CN(*)C[C@@H]2C)CC1]

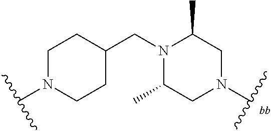 [SMILES: *N1CCC(CN2[C@@H](C)CN(*)C[C@@H]2C)CC1]

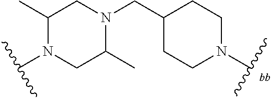 [SMILES: *N1CCC(CN2CC(C)N(*)CC2C)CC1]

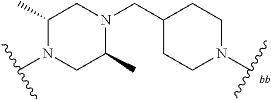 [SMILES: *N1CCC(CN2C[C@@H](C)N(*)C[C@@H]2C)CC1]

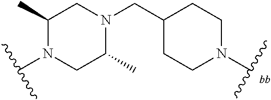 [SMILES: *N1CCC(CN2C[C@H](C)N(*)C[C@H]2C)CC1]

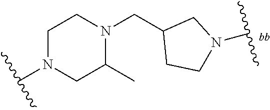 [SMILES: *N1CCC(CN2CCN(*)CC2C)C1]

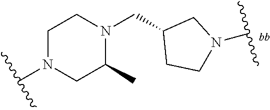 [SMILES: *N1CC[C@H](CN2CCN(*)C[C@@H]2C)C1]

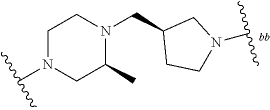 [SMILES: *N1CC[C@@H](CN2CCN(*)C[C@@H]2C)C1]

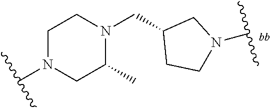 [SMILES: *N1CC[C@H](CN2CCN(*)C[C@H]2C)C1]

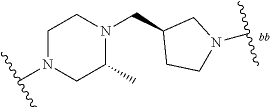 [SMILES: *N1CC[C@@H](CN2CCN(*)C[C@H]2C)C1]

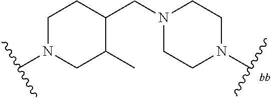 [SMILES: *N1CCN(CC2CCN(*)CC2C)CC1]

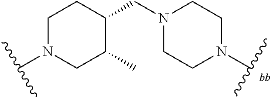 [SMILES: *N1CCN(C[C@H]2CCN(*)C[C@H]2C)CC1]

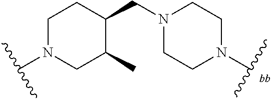 [SMILES: *N1CCN(C[C@@H]2CCN(*)C[C@@H]2C)CC1]

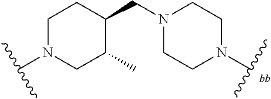 [SMILES: *N1CCN(C[C@@H]2CCN(*)C[C@H]2C)CC1]

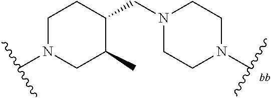 [SMILES: *N1CCN(C[C@H]2CCN(*)C[C@@H]2C)CC1]

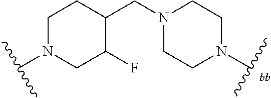 [SMILES: *N1CCN(CC2CCN(*)CC2F)CC1]

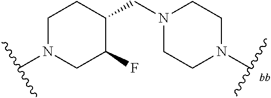 [SMILES: *N1CCN(C[C@H]2CCN(*)C[C@@H]2F)CC1]

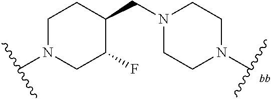 [SMILES: *N1CCN(C[C@@H]2CCN(*)C[C@H]2F)CC1]

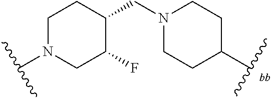 [SMILES: *C1CCN(C[C@H]2CCN(*)C[C@H]2F)CC1]

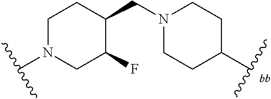 [SMILES: *C1CCN(C[C@@H]2CCN(*)C[C@@H]2F)CC1]

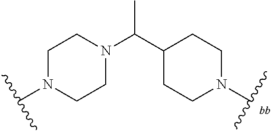 [SMILES: *N1CCC(C(C)N2CCN(*)CC2)CC1]

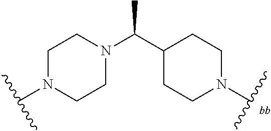 [SMILES: *N1CCC([C@H](C)N2CCN(*)CC2)CC1]

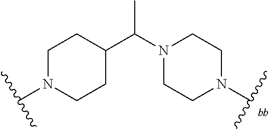 [SMILES: *N1CCC(C(C)N2CCN(*)CC2)CC1]

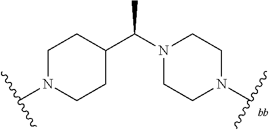 [SMILES: *N1CCC([C@@H](C)N2CCN(*)CC2)CC1]

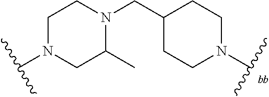 [SMILES: *N1CCC(CN2CCN(*)CC2C)CC1]

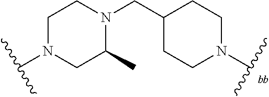 [SMILES: *N1CCC(CN2CCN(*)C[C@@H]2C)CC1]

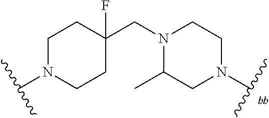 [SMILES: *N1CCC(F)(CN2CCN(*)CC2C)CC1]

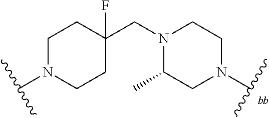 [SMILES: *N1CCC(F)(CN2CCN(*)C[C@@H]2C)CC1]

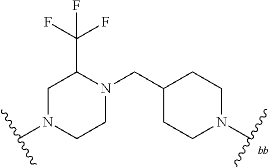 [SMILES: *N1CCC(CN2CCN(*)CC2C(F)(F)F)CC1]

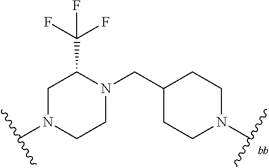 [SMILES: *N1CCC(CN2CCN(*)C[C@@H]2C(F)(F)F)CC1]

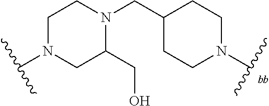 [SMILES: *N1CCC(CN2CCN(*)CC2CO)CC1]

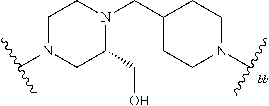 [SMILES: *N1CCC(CN2CCN(*)C[C@H]2CO)CC1]

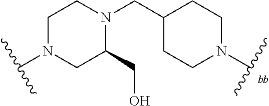 [SMILES: *N1CCC(CN2CCN(*)C[C@@H]2CO)CC1]

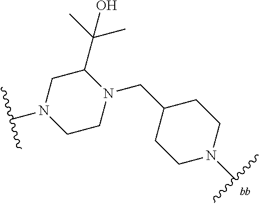 [SMILES: *N1CCC(CN2CCN(*)CC2C(C)(C)O)CC1]

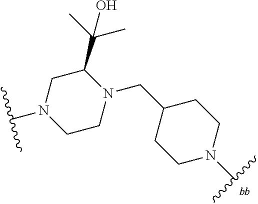 [SMILES: *N1CCC(CN2CCN(*)C[C@H]2C(C)(C)O)CC1]

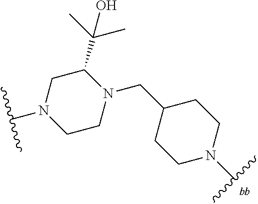 [SMILES: *N1CCC(CN2CCN(*)C[C@@H]2C(C)(C)O)CC1]

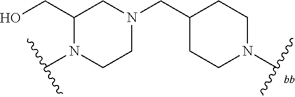 [SMILES: *N1CCC(CN2CCN(*)C(CO)C2)CC1]

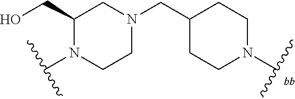 [SMILES: *N1CCC(CN2CCN(*)[C@@H](CO)C2)CC1]

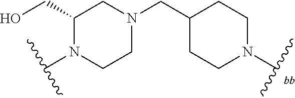 [SMILES: *N1CCC(CN2CCN(*)[C@H](CO)C2)CC1]

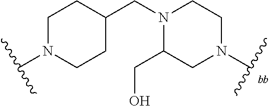 [SMILES: *N1CCC(CN2CCN(*)CC2CO)CC1]

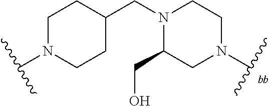 [SMILES: *N1CCC(CN2CCN(*)C[C@H]2CO)CC1]

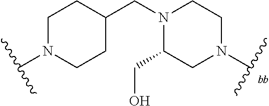 [SMILES: *N1CCC(CN2CCN(*)C[C@@H]2CO)CC1]

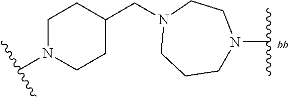 [SMILES: *N1CCC(CN2CCCN(*)CC2)CC1]

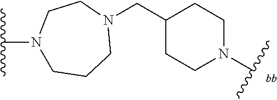 [SMILES: *N1CCC(CN2CCCN(*)CC2)CC1]

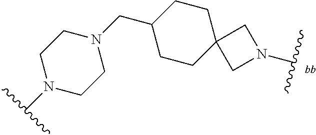 [SMILES: *N1CCN(CC2CCC3(CC2)CN(*)C3)CC1]

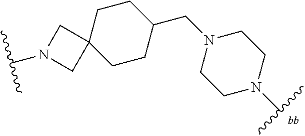 [SMILES: *N1CCN(CC2CCC3(CC2)CN(*)C3)CC1]

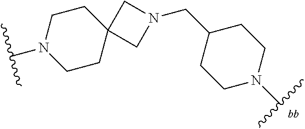 [SMILES: *N1CCC(CN2CC3(CCN(*)CC3)C2)CC1]

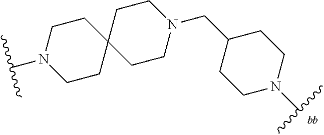 [SMILES: *N1CCC(CN2CCC3(CCN(*)CC3)CC2)CC1]

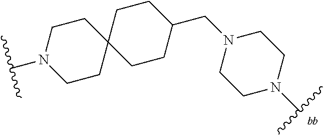 [SMILES: *N1CCN(CC2CCC3(CC2)CCN(*)CC3)CC1]

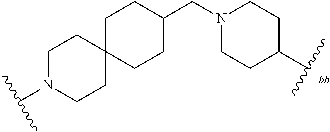 [SMILES: *C1CCN(CC2CCC3(CC2)CCN(*)CC3)CC1]

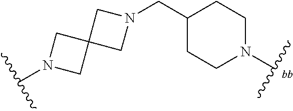 [SMILES: *N1CCC(CN2CC3(CN(*)C3)C2)CC1]

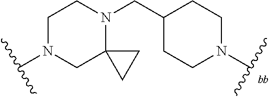 [SMILES: *N1CCC(CN2CCN(*)CC23CC3)CC1]

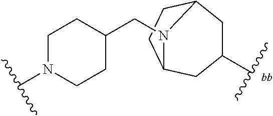 [SMILES: *C1CC2CCC(C1)N2CC1CCN(*)CC1]

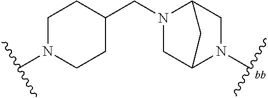 [SMILES: *N1CCC(CN2CC3CC2CN3*)CC1]

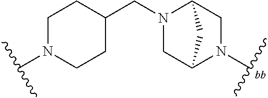 [SMILES: *N1CCC(CN2C[C@H]3C[C@@H]2CN3*)CC1]

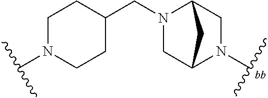 [SMILES: *N1CCC(CN2C[C@@H]3C[C@H]2CN3*)CC1]

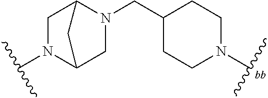 [SMILES: *N1CCC(CN2CC3CC2CN3*)CC1]

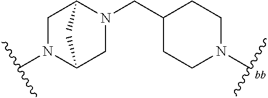 [SMILES: *N1CCC(CN2C[C@@H]3C[C@H]2CN3*)CC1]

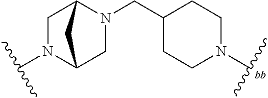 [SMILES: *N1CCC(CN2C[C@H]3C[C@@H]2CN3*)CC1]

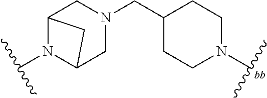 [SMILES: *N1CCC(CN2CC3CC(C2)N3*)CC1]

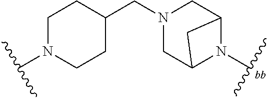 [SMILES: *N1CCC(CN2CC3CC(C2)N3*)CC1]

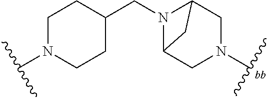 [SMILES: *N1CCC(CN2C3CC2CN(*)C3)CC1]

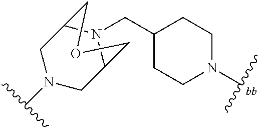 [SMILES: *N1CCC(CN2C3COCC2CN(*)C3)CC1]

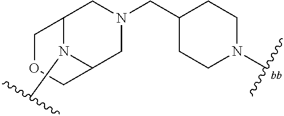 [SMILES: *N1CCC(CN2CC3COCC(C2)N3*)CC1]

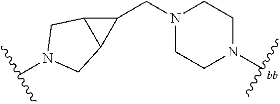 [SMILES: *N1CCN(CC2C3CN(*)CC32)CC1]

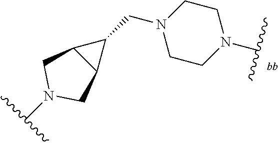 [SMILES: *N1CCN(C[C@H]2[C@@H]3CN(*)C[C@@H]32)CC1]

Example when L is -L-^{A4}-L^{A4}-_{bb}

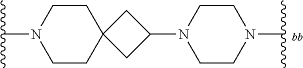 [SMILES: *N1CCN(C2CC3(CCN(*)CC3)C2)CC1]

Example when L is -L-^{A4}-L^{A1}-L^{A1}-L^{A4}-_{bb}

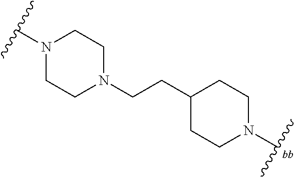 [SMILES: *N1CCC(CCN2CCN(*)CC2)CC1]

Examples when L is -L-^{A4}-L^{A3}-L^{A4}-_{bb}

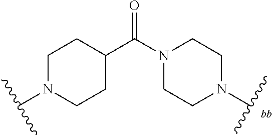 [SMILES: *N1CCC(C(=O)N2CCN(*)CC2)CC1]

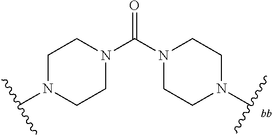 [SMILES: *N1CCN(C(=O)N2CCN(*)CC2)CC1]

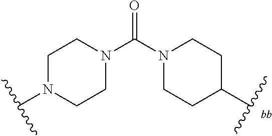 [SMILES: *C1CCN(C(=O)N2CCN(*)CC2)CC1]

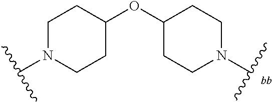 [SMILES: *N1CCC(OC2CCN(*)CC2)CC1]

Examples when L is -L-^{A4}-L^{A3}-_{bb}

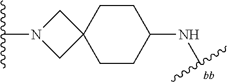 [SMILES: *NC1CCC2(CC1)CN(*)C2]

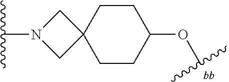 [SMILES: *OC1CCC2(CC1)CN(*)C2]

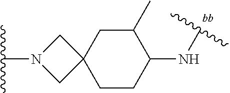 [SMILES: *NC1CCC2(CC1C)CN(*)C2]

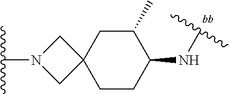 [SMILES: *N[C@H]1CCC2(C[C@@H]1C)CN(*)C2]

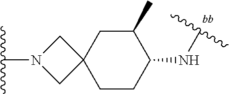 [SMILES: *N[C@@H]1CCC2(C[C@H]1C)CN(*)C2]

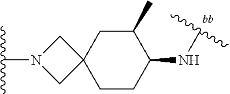 [SMILES: *N[C@H]1CCC2(C[C@H]1C)CN(*)C2]

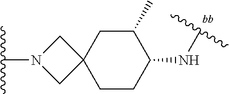 [SMILES: *N[C@@H]1CCC2(C[C@@H]1C)CN(*)C2]

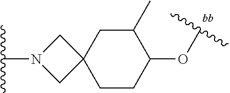 [SMILES: *OC1CCC2(CC1C)CN(*)C2]

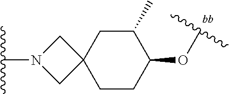 [SMILES: *O[C@H]1CCC2(C[C@@H]1C)CN(*)C2]

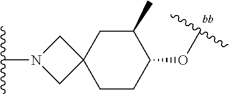 [SMILES: *O[C@@H]1CCC2(C[C@H]1C)CN(*)C2]

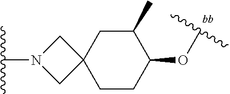 [SMILES: *O[C@H]1CCC2(C[C@H]1C)CN(*)C2]

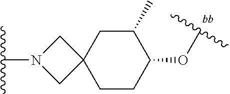 [SMILES: *O[C@@H]1CCC2(C[C@@H]1C)CN(*)C2]

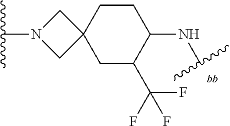 [SMILES: *NC1CCC2(CC1C(F)(F)F)CN(*)C2]

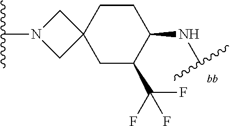 [SMILES: *N[C@@H]1CCC2(C[C@@H]1C(F)(F)F)CN(*)C2]

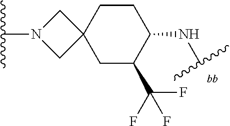 [SMILES: *N[C@H]1CCC2(C[C@@H]1C(F)(F)F)CN(*)C2]

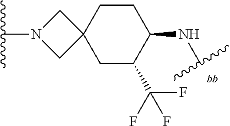 [SMILES: *N[C@@H]1CCC2(C[C@H]1C(F)(F)F)CN(*)C2]

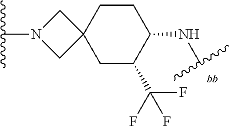 [SMILES: *N[C@H]1CCC2(C[C@H]1C(F)(F)F)CN(*)C2]

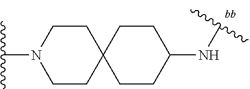 [SMILES: *NC1CCC2(CC1)CCN(*)CC2]

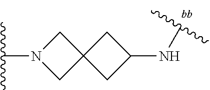 [SMILES: *NC1CC2(C1)CN(*)C2]

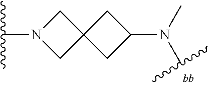 [SMILES: *N1CC2(CC(N(*)C)C2)C1]

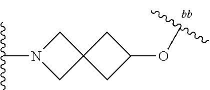 [SMILES: *OC1CC2(C1)CN(*)C2]

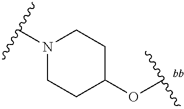 [SMILES: *OC1CCN(*)CC1]

Examples when L is -L-^{A4}-L^{A1}-L^{A3}-_{bb}

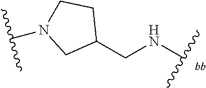 [SMILES: *NCC1CCN(*)C1]

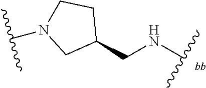 [SMILES: *NC[C@@H]1CCN(*)C1]

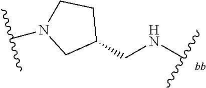 [SMILES: *NC[C@H]1CCN(*)C1]

Examples when L is -L-^{A4}-L^{A4}-L^{A3}-L^{A4}-_{bb} or -L-^{A4}-L^{A3}-L^{A4}-L^{AA}-_{bb}

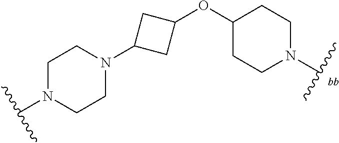 [SMILES: *N1CCC(OC2CC(N3CCN(*)CC3)C2)CC1]

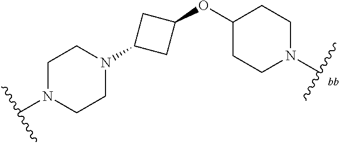 [SMILES: *N1CCC(O[C@H]2C[C@H](N3CCN(*)CC3)C2)CC1]

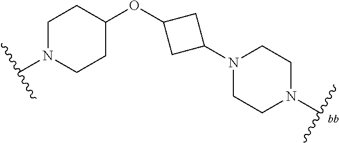 [SMILES: *N1CCC(OC2CC(N3CCN(*)CC3)C2)CC1]

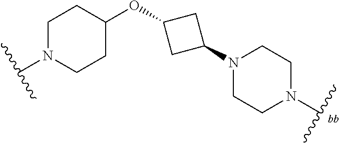 [SMILES: *N1CCC(O[C@H]2C[C@H](N3CCN(*)CC3)C2)CC1]

Examples when L is -L-^{A3}-L^{A4}-_{bb} or -L^{A4}-

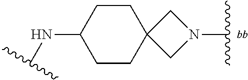 [SMILES: *NC1CCC2(CC1)CN(*)C2]

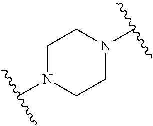 [SMILES: *N1CCN(*)CC1]

In some embodiments, L is selected from the group consisting of the moieties delineated in Table L1a:

TABLE L1a

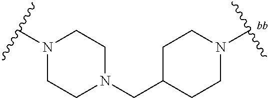

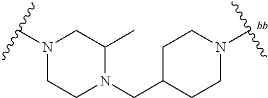

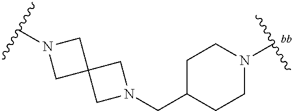

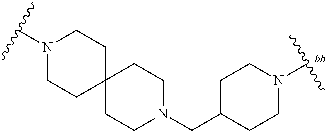

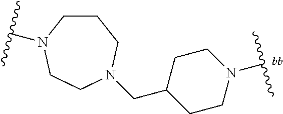

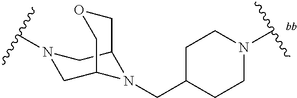

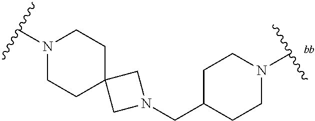

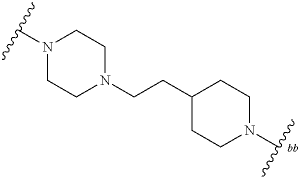

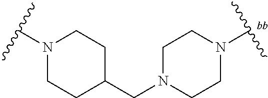

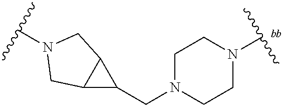

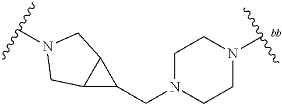

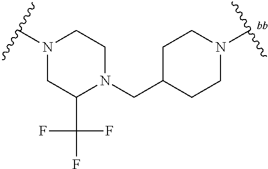

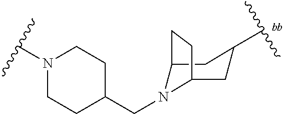

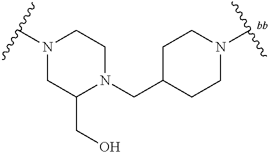

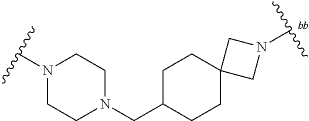

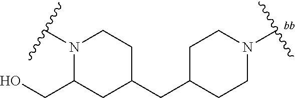

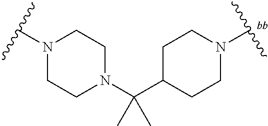

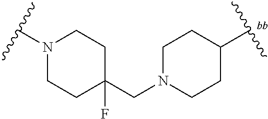

wherein bb represents the point of attachment to Ring C.

In some embodiments, L is selected from the group consisting of the moieties delineated in Table L3a:

TABLE L3a

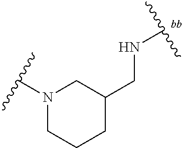

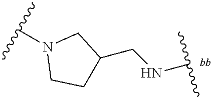

wherein bb represents the point of attachment to Ring C.

In some embodiments, L is selected from the group consisting of the moieties delineated in Table L4a:

TABLE L4a

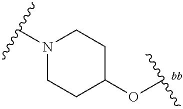

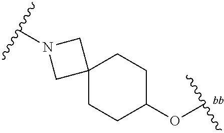

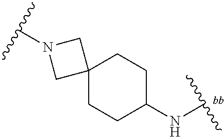

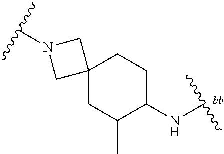

wherein bb represents the point of attachment to Ring C.

In some embodiments, L is selected from the group consisting of:

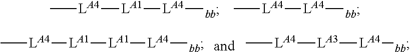

wherein bb represents the point of attachment to Ring C; and
L^{A1} is —CH₂—, —CHMe-, —CMe₂-, or —CH(OH)—;
each L^{A4} is independently a 4-12 membered nitrogen-containing heterocyclylene optionally substituted with 1-3 R^{a}, wherein:
each R^{a} present on L^{A4} is independently selected from the group consisting of: —F; CN; C₁₋₃ alkoxy; OH; C₁₋₃ alkyl substituted with OH; and C₁₋₃ alkyl optionally substituted with 1-3 F.

In some embodiments, L is selected from the group consisting of:

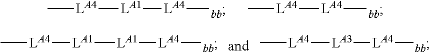

wherein bb represents the point of attachment to Ring C; and
L^{A1} is CH₂, CHMe, or CMe₂;
each L^{A4} is independently 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments, L is L^{A4}-L^{A1}-L^{A4}-_{bb}, wherein:
bb represents the point of attachment to Ring C;
L^{A1} is —CH₂—, —CHMe-, —CMe₂-, or —CH(OH)—;
each L^{A4} is independently 4-12 membered heterocyclylene, each of which is optionally substituted with 1-3 R^{a}; and
each R^{a} present on L^{A4} is independently selected from the group consisting of: —F; CN; C₁₋₃ alkoxy; OH; C₁₋₃ alkyl substituted with OH; and C₁₋₃ alkyl optionally substituted with 1-3 F.

In some embodiments, L is L^{A4}-L^{A1}-L^{A4}-_{bb}, wherein:
bb represents the point of attachment to Ring C;
L^{A1} is CH₂, CHMe, or CMe₂;
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments, the

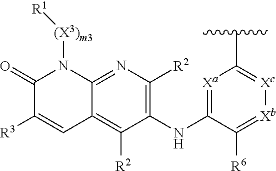

moiety is:

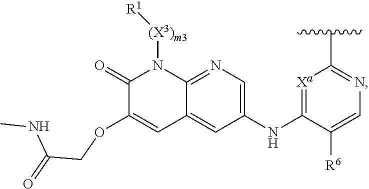

wherein:
R⁶ is —F or —Cl;
X^{a} is N or CH;
L is selected from the group consisting of:

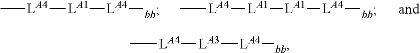

wherein bb represents the point of attachment to Ring C, wherein:
each L^{A4} is independently a monocyclic 4-6 membered nitrogen-containing heterocyclylene optionally substituted with 1-3 R^{a}; and
the

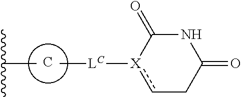

moiety is selected from the group consisting of:

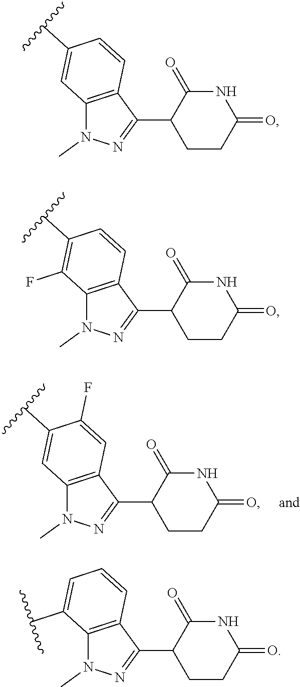

In some embodiments, m3 is 1; X³ is methylene, ethylene, or isopropylene; and R¹ is H. In some embodiments, X^{a} is N. In some embodiments, X^{a} is N. In some embodiments, the

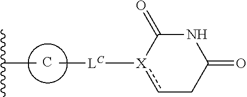

moiety is selected from the group consisting of:

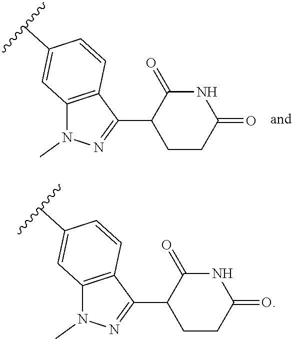 [SMILES: *c1ccc2c(C3CCC(=O)NC3=O)nn(C)c2c1 ; *c1ccc2c(C3CCC(=O)NC3=O)nn(C)c2c1]

In some embodiments, the

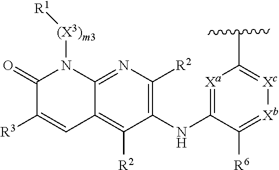

moiety is:

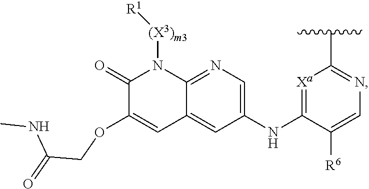

wherein:
R⁶ is —F or —Cl;
X^{a} is N or CH;
L is selected from the group consisting of:

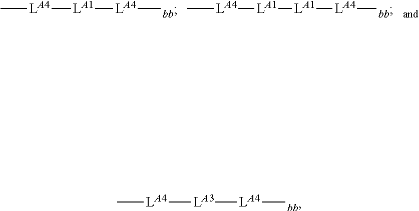

wherein bb represents the point of attachment to Ring C, wherein:
one L^{A4} is a monocyclic 4-6 membered nitrogen-containing heterocyclylene optionally substituted with 1-3 R^{a}; and the other L^{A4} is a bicyclic 6-12 membered nitrogen-containing heterocyclylene optionally substituted with 1-3 R^{a}; and
the

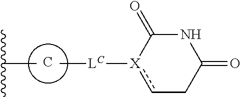

moiety is selected from the group consisting of:

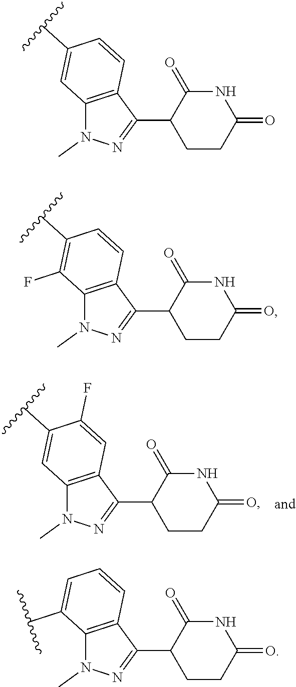

In some embodiments, m3 is 1; X³ is methylene, ethylene, or isopropylene; and R¹ is H. In some embodiments, X^{a} is N. In some embodiments, the

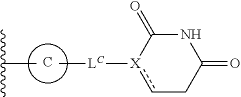

moiety is selected from the group consisting of:

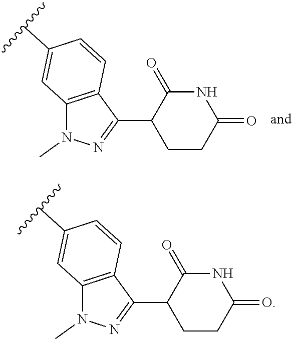 [SMILES: *c1ccc2c(C3CCC(=O)NC3=O)nn(C)c2c1 ; *c1ccc2c(C3CCC(=O)NC3=O)nn(C)c2c1]

In some embodiments, the

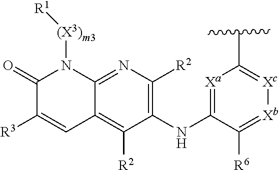

moiety is:

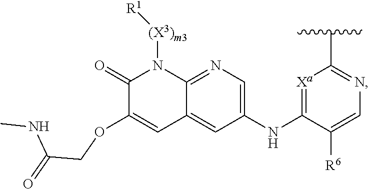

wherein:
R⁶ is —F or —Cl;
X^{a} is N or CH;
L is selected from the group consisting of the moieties delineated in Table L, Table L1a or Table L3a (e.g., Table L1a or Table L3a); and
the

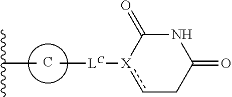

moiety is selected from the group consisting of:

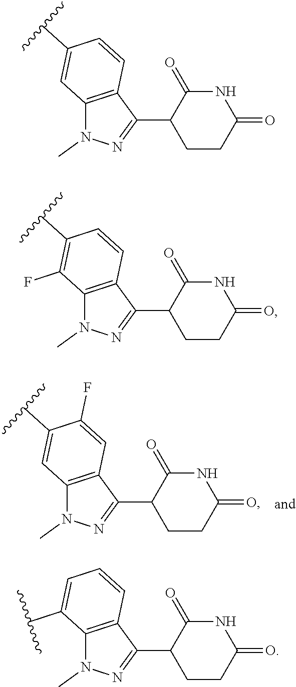

In some embodiments, m3 is 1; X³ is methylene, ethylene, or isopropylene; and R¹ is H. In some embodiments, X^{a} is N. In some embodiments, X^{a} is CH. In some embodiments, the

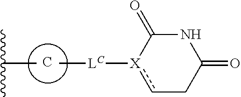

moiety is selected from the group consisting of:

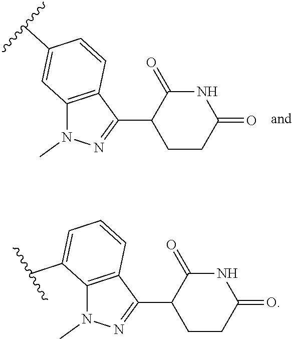 [SMILES: *c1ccc2c(C3CCC(=O)NC3=O)nn(C)c2c1 ; *c1cccc2c(C3CCC(=O)NC3=O)nn(C)c12]

In some embodiments, the compounds of Formula (I) are compounds of Formula (I-a):

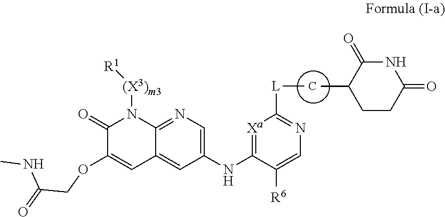

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

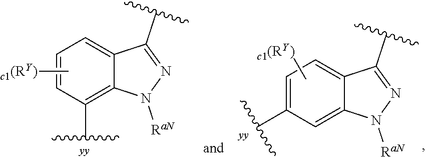

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L is selected from the group consisting of:

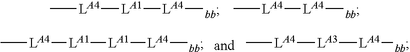

wherein bb represents the point of attachment to Ring C; and
L^{A1} is CH₂, CHMe, or CMe₂;
each L^{A4} is independently 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-a), each L^{A4} is independently monocyclic 4-6 membered nitrogen-containing heterocyclylene optionally substituted with 1-3 R^{a}, wherein each L^{A4} contains 1-2 ring nitrogen atoms and no additional ring heteroatoms.

In some embodiments of Formula (I-a), one L^{A4} is a monocyclic 4-6 membered nitrogen-containing heterocyclylene optionally substituted with 1-3 R^{a}; and
the other L^{A4} is a bicyclic spirocyclic 6-12 (e.g., 6-10) membered nitrogen-containing heterocyclylene optionally substituted with 1-3 R^{a},
wherein each L^{A4} contains 1-2 ring nitrogen atoms and no additional ring heteroatoms.

In some embodiments of Formula (I-a), L is -L^{A4}-L^{A1}-L^{A4}-_{bb}; and L^{A1} is CH₂ or CHMe.

In some embodiments of Formula (I-a), L is selected from the group consisting of the moieties delineated in Table L-I-a, wherein bb represents the point of attachment to Ring C.

TABLE L-I-a

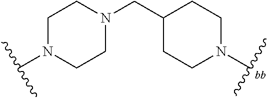 [SMILES: *N1CCC(CN2CCN(*)CC2)CC1]

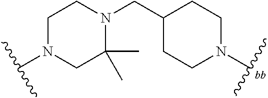 [SMILES: *N1CCC(CN2CCN(*)CC2(C)C)CC1]

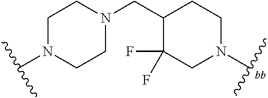 [SMILES: *N1CCN(CC2CCN(*)CC2(F)F)CC1]

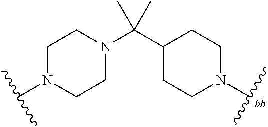 [SMILES: *N1CCC(C(C)(C)N2CCN(*)CC2)CC1]

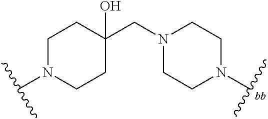 [SMILES: *N1CCN(CC2(O)CCN(*)CC2)CC1]

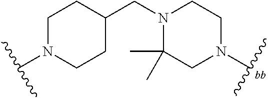 [SMILES: *N1CCC(CN2CCN(*)CC2(C)C)CC1]

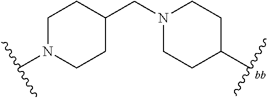 [SMILES: *C1CCN(CC2CCN(*)CC2)CC1]

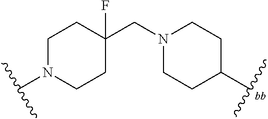 [SMILES: *C1CCN(CC2(F)CCN(*)CC2)CC1]

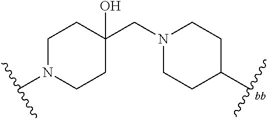 [SMILES: *C1CCN(CC2(O)CCN(*)CC2)CC1]

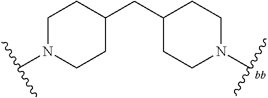 [SMILES: *N1CCC(CC2CCN(*)CC2)CC1]

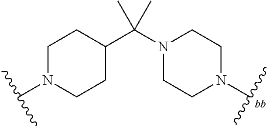 [SMILES: *N1CCC(C(C)(C)N2CCN(*)CC2)CC1]

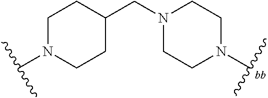 [SMILES: *N1CCC(CN2CCN(*)CC2)CC1]

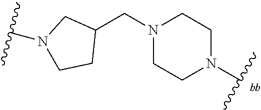 [SMILES: *N1CCN(CC2CCN(*)C2)CC1]

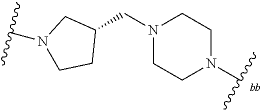 [SMILES: *N1CCN(C[C@@H]2CCN(*)C2)CC1]

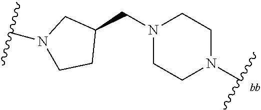 [SMILES: *N1CCN(C[C@H]2CCN(*)C2)CC1]

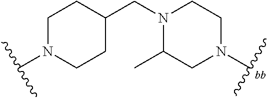 [SMILES: *N1CCC(CN2CCN(*)CC2C)CC1]

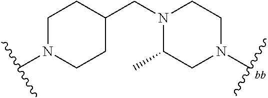 [SMILES: *N1CCC(CN2CCN(*)C[C@@H]2C)CC1]

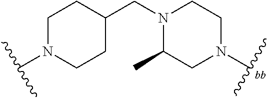 [SMILES: *N1CCC(CN2CCN(*)C[C@H]2C)CC1]

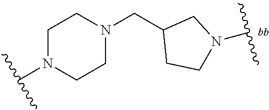 [SMILES: *N1CCN(CC2CCN(*)C2)CC1]

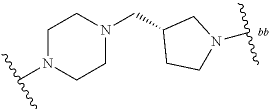 [SMILES: *N1CCN(C[C@H]2CCN(*)C2)CC1]

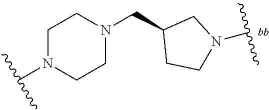 [SMILES: *N1CCN(C[C@@H]2CCN(*)C2)CC1]

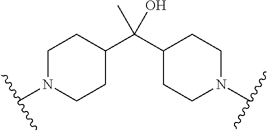 [SMILES: *N1CCC(C(C)(O)C2CCN(*)CC2)CC1]

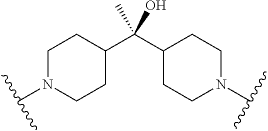

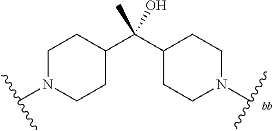

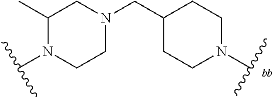 [SMILES: *N1CCC(CN2CCN(*)C(C)C2)CC1]

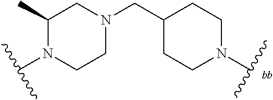 [SMILES: *N1CCC(CN2CCN(*)[C@@H](C)C2)CC1]

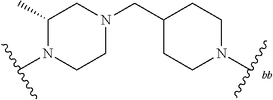 [SMILES: *N1CCC(CN2CCN(*)[C@H](C)C2)CC1]

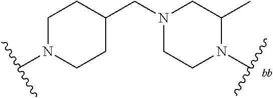 [SMILES: *N1CCC(CN2CCN(*)C(C)C2)CC1]

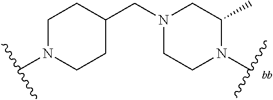 [SMILES: *N1CCC(CN2CCN(*)[C@@H](C)C2)CC1]

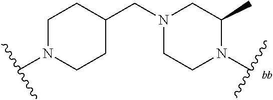 [SMILES: *N1CCC(CN2CCN(*)[C@H](C)C2)CC1]

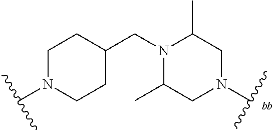 [SMILES: *N1CCC(CN2C(C)CN(*)CC2C)CC1]

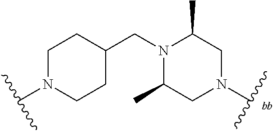 [SMILES: *N1CCC(CN2[C@H](C)CN(*)C[C@@H]2C)CC1]

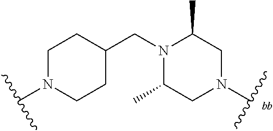 [SMILES: *N1CCC(CN2[C@@H](C)CN(*)C[C@@H]2C)CC1]

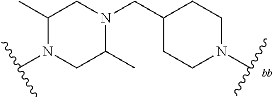 [SMILES: *N1CCC(CN2CC(C)N(*)CC2C)CC1]

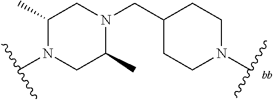 [SMILES: *N1CCC(CN2C[C@@H](C)N(*)C[C@@H]2C)CC1]

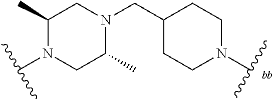 [SMILES: *N1CCC(CN2C[C@H](C)N(*)C[C@H]2C)CC1]

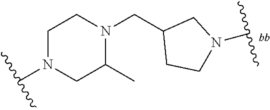 [SMILES: *N1CCC(CN2CCN(*)CC2C)C1]

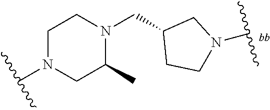 [SMILES: *N1CC[C@H](CN2CCN(*)C[C@@H]2C)C1]

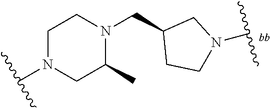 [SMILES: *N1CC[C@@H](CN2CCN(*)C[C@@H]2C)C1]

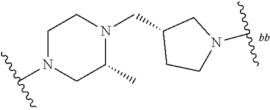 [SMILES: *N1CC[C@H](CN2CCN(*)C[C@H]2C)C1]

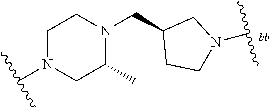 [SMILES: *N1CC[C@@H](CN2CCN(*)C[C@H]2C)C1]

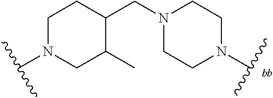 [SMILES: *N1CCN(CC2CCN(*)CC2C)CC1]

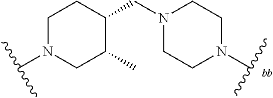 [SMILES: *N1CCN(C[C@H]2CCN(*)C[C@H]2C)CC1]

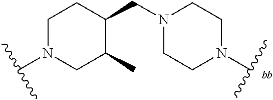 [SMILES: *N1CCN(C[C@@H]2CCN(*)C[C@@H]2C)CC1]

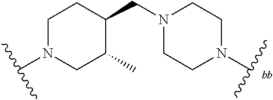 [SMILES: *N1CCN(C[C@@H]2CCN(*)C[C@H]2C)CC1]

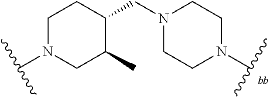 [SMILES: *N1CCN(C[C@H]2CCN(*)C[C@@H]2C)CC1]

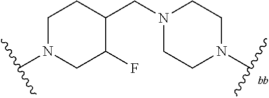 [SMILES: *N1CCN(CC2CCN(*)CC2F)CC1]

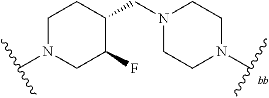 [SMILES: *N1CCN(C[C@H]2CCN(*)C[C@@H]2F)CC1]

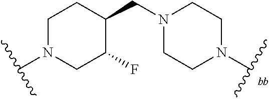 [SMILES: *N1CCN(C[C@@H]2CCN(*)C[C@H]2F)CC1]

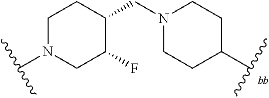 [SMILES: *C1CCN(C[C@H]2CCN(*)C[C@H]2F)CC1]

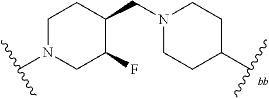 [SMILES: *C1CCN(C[C@@H]2CCN(*)C[C@@H]2F)CC1]

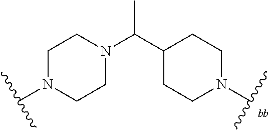 [SMILES: *N1CCC(C(C)N2CCN(*)CC2)CC1]

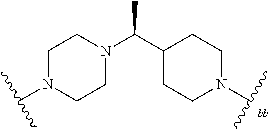 [SMILES: *N1CCC([C@H](C)N2CCN(*)CC2)CC1]

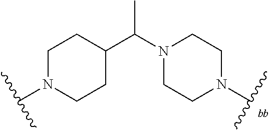 [SMILES: *N1CCC(C(C)N2CCN(*)CC2)CC1]

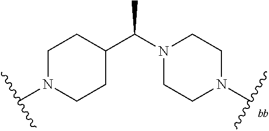 [SMILES: *N1CCC([C@@H](C)N2CCN(*)CC2)CC1]

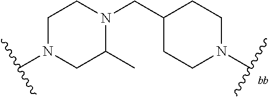 [SMILES: *N1CCC(CN2CCN(*)CC2C)CC1]

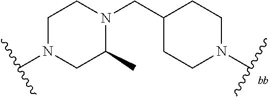 [SMILES: *N1CCC(CN2CCN(*)C[C@@H]2C)CC1]

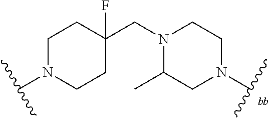 [SMILES: *N1CCC(F)(CN2CCN(*)CC2C)CC1]

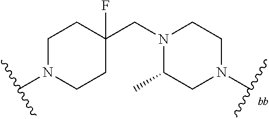 [SMILES: *N1CCC(F)(CN2CCN(*)C[C@@H]2C)CC1]

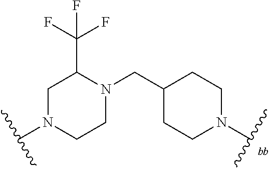 [SMILES: *N1CCC(CN2CCN(*)CC2C(F)(F)F)CC1]

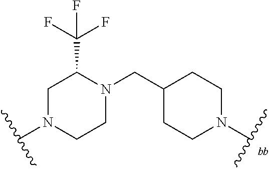 [SMILES: *N1CCC(CN2CCN(*)C[C@@H]2C(F)(F)F)CC1]

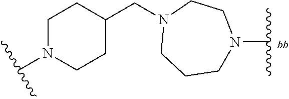 [SMILES: *N1CCC(CN2CCCN(*)CC2)CC1]

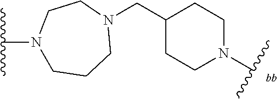 [SMILES: *N1CCC(CN2CCCN(*)CC2)CC1]

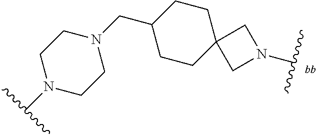 [SMILES: *N1CCN(CC2CCC3(CC2)CN(*)C3)CC1]

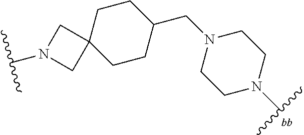 [SMILES: *N1CCN(CC2CCC3(CC2)CN(*)C3)CC1]

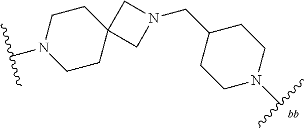 [SMILES: *N1CCC(CN2CC3(CCN(*)CC3)C2)CC1]

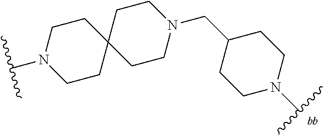 [SMILES: *N1CCC(CN2CCC3(CCN(*)CC3)CC2)CC1]

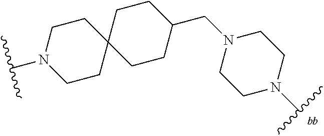 [SMILES: *N1CCN(CC2CCC3(CC2)CCN(*)CC3)CC1]

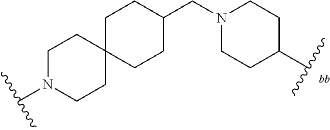 [SMILES: *C1CCN(CC2CCC3(CC2)CCN(*)CC3)CC1]

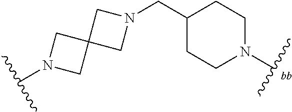 [SMILES: *N1CCC(CN2CC3(CN(*)C3)C2)CC1]

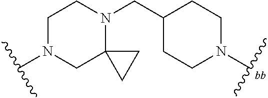 [SMILES: *N1CCC(CN2CCN(*)CC23CC3)CC1]

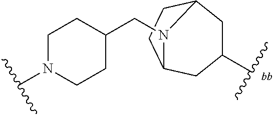 [SMILES: *C1CC2CCC(C1)N2CC1CCN(*)CC1]

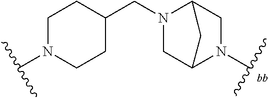 [SMILES: *N1CCC(CN2CC3CC2CN3*)CC1]

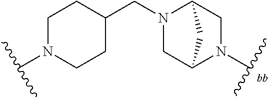 [SMILES: *N1CCC(CN2C[C@H]3C[C@@H]2CN3*)CC1]

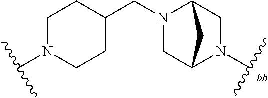 [SMILES: *N1CCC(CN2C[C@@H]3C[C@H]2CN3*)CC1]

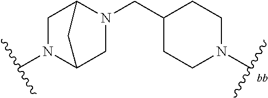 [SMILES: *N1CCC(CN2CC3CC2CN3*)CC1]

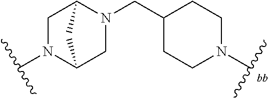 [SMILES: *N1CCC(CN2C[C@@H]3C[C@H]2CN3*)CC1]

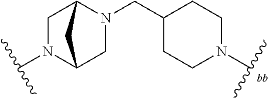 [SMILES: *N1CCC(CN2C[C@H]3C[C@@H]2CN3*)CC1]

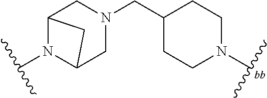 [SMILES: *N1CCC(CN2CC3CC(C2)N3*)CC1]

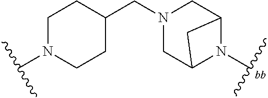 [SMILES: *N1CCC(CN2CC3CC(C2)N3*)CC1]

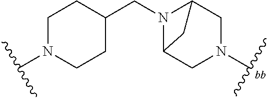 [SMILES: *N1CCC(CN2C3CC2CN(*)C3)CC1]

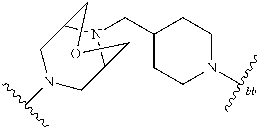 [SMILES: *N1CCC(CN2C3COCC2CN(*)C3)CC1]

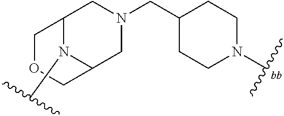 [SMILES: *N1CCC(CN2CC3COCC(C2)N3*)CC1]

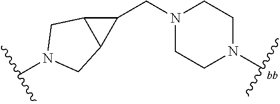 [SMILES: *N1CCN(CC2C3CN(*)CC32)CC1]

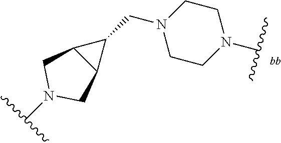 [SMILES: *N1CCN(C[C@H]2[C@@H]3CN(*)C[C@@H]32)CC1]

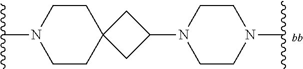 [SMILES: *N1CCN(C2CC3(CCN(*)CC3)C2)CC1]

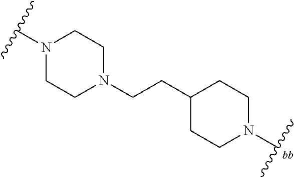 [SMILES: *N1CCC(CCN2CCN(*)CC2)CC1]

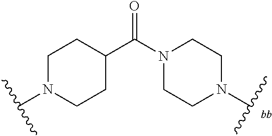 [SMILES: *N1CCC(C(=O)N2CCN(*)CC2)CC1]

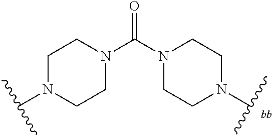 [SMILES: *N1CCN(C(=O)N2CCN(*)CC2)CC1]

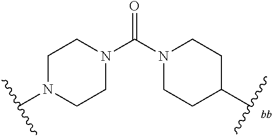 [SMILES: *C1CCN(C(=O)N2CCN(*)CC2)CC1]

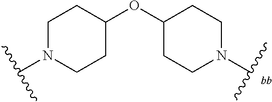 [SMILES: *N1CCC(OC2CCN(*)CC2)CC1]

In some embodiments, the compounds of Formula (I-a) are compounds of Formula (I-a-1):

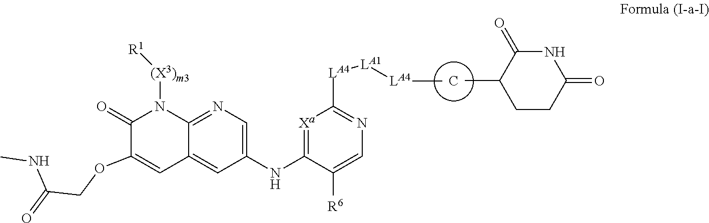

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

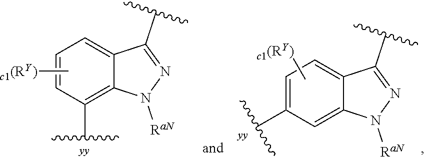

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂; and
each L^{A4} is independently monocyclic 4-6 membered nitrogen-containing heterocyclylene optionally substituted with 1-3 R^{a}, wherein:
each L^{A4} contains 1-2 ring nitrogen atoms and no additional ring heteroatoms, and
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-a-1), the -L^{A4}-L^{A1}-L^{A4}- moiety is selected from the group consisting of the moieties delineated in Table L-I-a-1, wherein bb represents the point of attachment to Ring C.

TABLE L-I-a-1

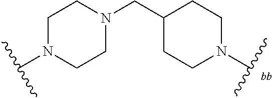 [SMILES: *N1CCC(CN2CCN(*)CC2)CC1]

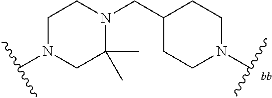 [SMILES: *N1CCC(CN2CCN(*)CC2(C)C)CC1]

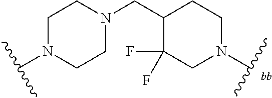 [SMILES: *N1CCN(CC2CCN(*)CC2(F)F)CC1]

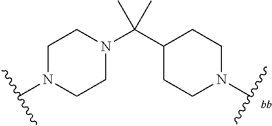 [SMILES: *N1CCC(C(C)(C)N2CCN(*)CC2)CC1]

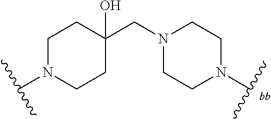 [SMILES: *N1CCN(CC2(O)CCN(*)CC2)CC1]

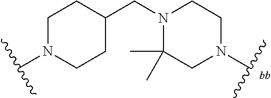 [SMILES: *N1CCC(CN2CCN(*)CC2(C)C)CC1]

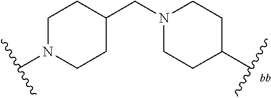 [SMILES: *C1CCN(CC2CCN(*)CC2)CC1]

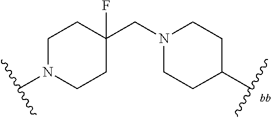 [SMILES: *C1CCN(CC2(F)CCN(*)CC2)CC1]

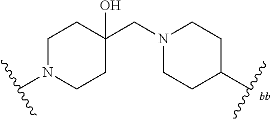 [SMILES: *C1CCN(CC2(O)CCN(*)CC2)CC1]

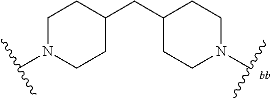 [SMILES: *N1CCC(CC2CCN(*)CC2)CC1]

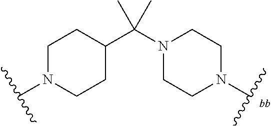 [SMILES: *N1CCC(C(C)(C)N2CCN(*)CC2)CC1]

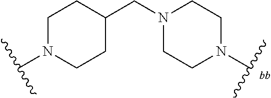 [SMILES: *N1CCC(CN2CCN(*)CC2)CC1]

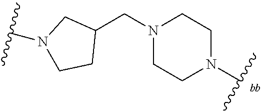 [SMILES: *N1CCN(CC2CCN(*)C2)CC1]

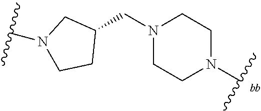 [SMILES: *N1CCN(C[C@@H]2CCN(*)C2)CC1]

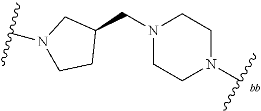 [SMILES: *N1CCN(C[C@H]2CCN(*)C2)CC1]

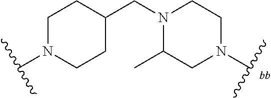 [SMILES: *N1CCC(CN2CCN(*)CC2C)CC1]

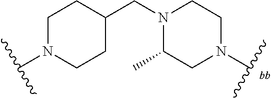 [SMILES: *N1CCC(CN2CCN(*)C[C@@H]2C)CC1]

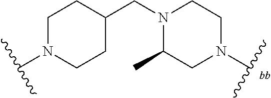 [SMILES: *N1CCC(CN2CCN(*)C[C@H]2C)CC1]

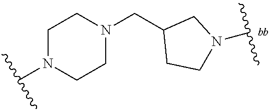 [SMILES: *N1CCN(CC2CCN(*)C2)CC1]

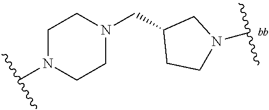 [SMILES: *N1CCN(C[C@H]2CCN(*)C2)CC1]

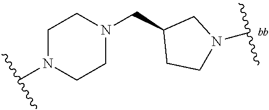 [SMILES: *N1CCN(C[C@@H]2CCN(*)C2)CC1]

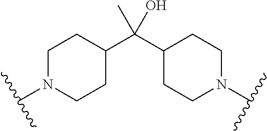 [SMILES: *N1CCC(C(C)(O)C2CCN(*)CC2)CC1]

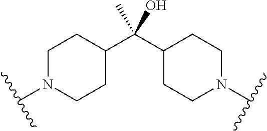

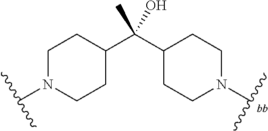

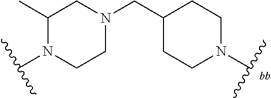 [SMILES: *N1CCC(CN2CCN(*)C(C)C2)CC1]

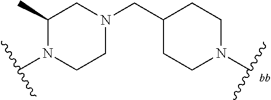 [SMILES: *N1CCC(CN2CCN(*)[C@@H](C)C2)CC1]

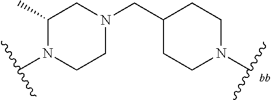 [SMILES: *N1CCC(CN2CCN(*)[C@H](C)C2)CC1]

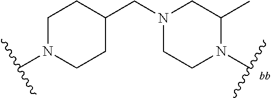 [SMILES: *N1CCC(CN2CCN(*)C(C)C2)CC1]

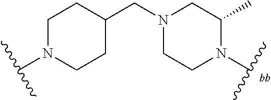 [SMILES: *N1CCC(CN2CCN(*)[C@@H](C)C2)CC1]

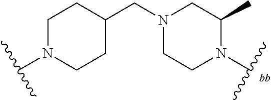 [SMILES: *N1CCC(CN2CCN(*)[C@H](C)C2)CC1]

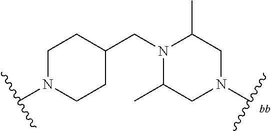 [SMILES: *N1CCC(CN2C(C)CN(*)CC2C)CC1]

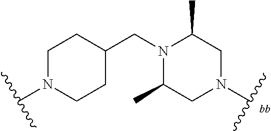 [SMILES: *N1CCC(CN2[C@H](C)CN(*)C[C@@H]2C)CC1]

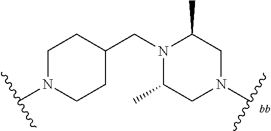 [SMILES: *N1CCC(CN2[C@@H](C)CN(*)C[C@@H]2C)CC1]

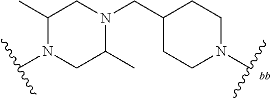 [SMILES: *N1CCC(CN2CC(C)N(*)CC2C)CC1]

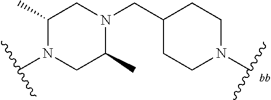 [SMILES: *N1CCC(CN2C[C@@H](C)N(*)C[C@@H]2C)CC1]

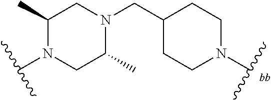 [SMILES: *N1CCC(CN2C[C@H](C)N(*)C[C@H]2C)CC1]

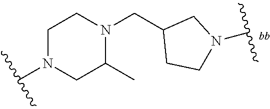 [SMILES: *N1CCC(CN2CCN(*)CC2C)C1]

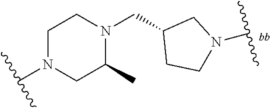 [SMILES: *N1CC[C@H](CN2CCN(*)C[C@@H]2C)C1]

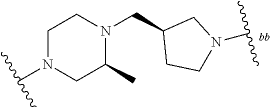 [SMILES: *N1CC[C@@H](CN2CCN(*)C[C@@H]2C)C1]

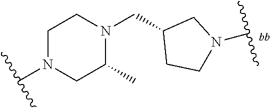 [SMILES: *N1CC[C@H](CN2CCN(*)C[C@H]2C)C1]

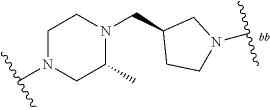 [SMILES: *N1CC[C@@H](CN2CCN(*)C[C@H]2C)C1]

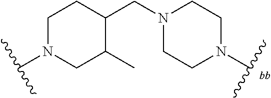 [SMILES: *N1CCN(CC2CCN(*)CC2C)CC1]

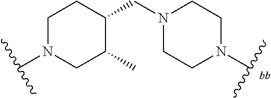 [SMILES: *N1CCN(C[C@H]2CCN(*)C[C@H]2C)CC1]

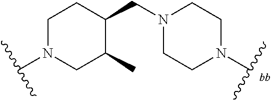 [SMILES: *N1CCN(C[C@@H]2CCN(*)C[C@@H]2C)CC1]

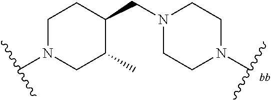 [SMILES: *N1CCN(C[C@@H]2CCN(*)C[C@H]2C)CC1]

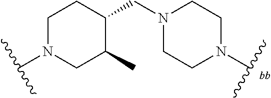 [SMILES: *N1CCN(C[C@H]2CCN(*)C[C@@H]2C)CC1]

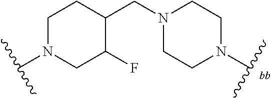 [SMILES: *N1CCN(CC2CCN(*)CC2F)CC1]

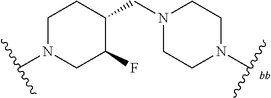 [SMILES: *N1CCN(C[C@H]2CCN(*)C[C@@H]2F)CC1]

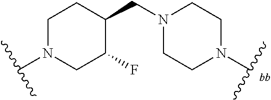 [SMILES: *N1CCN(C[C@@H]2CCN(*)C[C@H]2F)CC1]

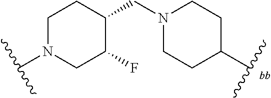 [SMILES: *C1CCN(C[C@H]2CCN(*)C[C@H]2F)CC1]

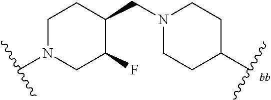 [SMILES: *C1CCN(C[C@@H]2CCN(*)C[C@@H]2F)CC1]

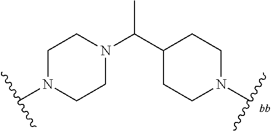 [SMILES: *N1CCC(C(C)N2CCN(*)CC2)CC1]

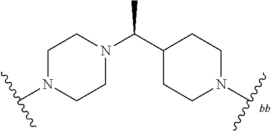 [SMILES: *N1CCC([C@H](C)N2CCN(*)CC2)CC1]

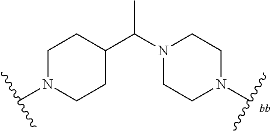 [SMILES: *N1CCC(C(C)N2CCN(*)CC2)CC1]

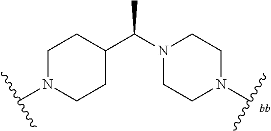 [SMILES: *N1CCC([C@@H](C)N2CCN(*)CC2)CC1]

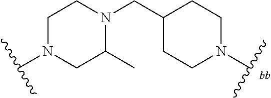 [SMILES: *N1CCC(CN2CCN(*)CC2C)CC1]

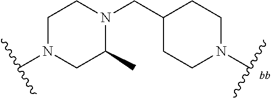 [SMILES: *N1CCC(CN2CCN(*)C[C@@H]2C)CC1]

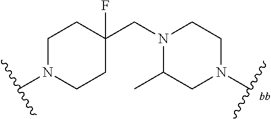 [SMILES: *N1CCC(F)(CN2CCN(*)CC2C)CC1]

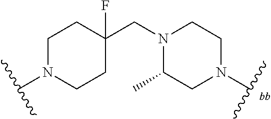 [SMILES: *N1CCC(F)(CN2CCN(*)C[C@@H]2C)CC1]

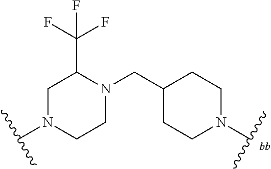 [SMILES: *N1CCC(CN2CCN(*)CC2C(F)(F)F)CC1]

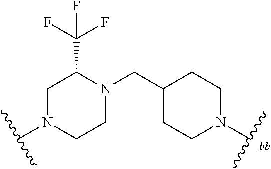 [SMILES: *N1CCC(CN2CCN(*)C[C@@H]2C(F)(F)F)CC1]

In some embodiments, the compounds of Formula (I-a) are compounds of Formula (I-a-2):

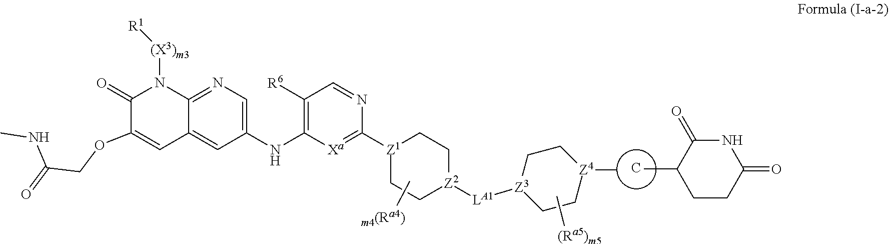

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

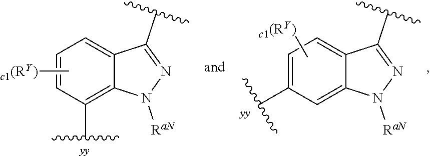

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂;
Z1 and Z² are independently selected from the group consisting of: CH, CR^{a4}, and N;
Z3 and Z4 are independently selected from the group consisting of: CH, CR^{a5}, and N, provided that at least one of Z¹ and Z² is N; at least one of Z³ and Z⁴ is N; and when Z² is N, then Z³ is CH or CR^{a5};
m4 and m5 are independently selected from the group consisting of: 0, 1, and 2; and
each R^{a4} and R^{a5} is independently selected from the group consisting of: —F, CN, C₁₋₃ alkoxy, OH, and C₁₋₃ alkyl optionally substituted with 1-3 F.

In some embodiments of Formula (I-a-2), Z¹ is N. In some embodiments of Formula (I-a-2), Z¹ is N; and Z² is CH or CR^{a4}. In some embodiments of Formula (I-a-2), Z¹ is N; Z² is CH or CR^{a4}; and Z³ is N.

In some embodiments of Formula (I-a-2), Z¹ is N; Z² is CH or CR^{a4}; Z³ is N; and Z⁴ is CH or CR^{a5}.

In some embodiments of Formula (I-a-2), Z¹ is N; Z² is CH; Z³ is N; Z⁴ is CH; and m4 and m5 are both 0. In some embodiments of Formula (I-a-2), Z¹ is N; Z² is CH; Z³ is N; Z⁴ is CH; one of m4 and m5 is 1; and the other of m4 and m5 is 0. In some embodiments of Formula (I-a-2), Z¹ is N; Z² is CH; Z³ is N; Z⁴ is CH; one of m4 and m5 is 1; the other of m4 and m5 is 0; and the R^{a4} or R^{a5} when present is methyl. In some embodiments of Formula (I-a-2), Z¹ is N; Z² is CH; Z³ is N; Z⁴ is CH; m4 is 1; and m5 is 0. In some embodiments of Formula (I-a-2), Z¹ is N; Z² is CH; Z³ is N; Z⁴ is CH; m4 is 1; m5 is 0; and R^{a4} is methyl.

In some embodiments of Formula (I-a-2), Z¹ is N; Z² is CH; Z³ is N; and Z⁴ is CR^{a5} (e.g., CF). In some embodiments of Formula (I-a-2), Z¹ is N; Z² is CH; Z³ is N; Z⁴ is CF; and m4 and m5 are both 0.

In some embodiments of Formula (I-a-2), Z¹ is N; Z² is CH or CR^{a4}; Z³ is N; and Z⁴ is N. In some embodiments of Formula (I-a-2), Z¹ is N; Z² is CH; Z³ is N; Z⁴ is N; and m4 and m5 are both 0. In some embodiments of Formula (I-a-2), Z¹ is N; Z² is CH; Z³ is N; Z⁴ is N; one of m4 and m5 is 1; and the other of m4 and m5 is 0. In some embodiments of Formula (I-a-2), Z¹ is N; Z² is CH; Z³ is N; Z⁴ is N; one of m4 and m5 is 1; the other of m4 and m5 is 0; and the R^{a4} or R^{a5} when present is methyl. In some embodiments of Formula (I-a-2), Z¹ is N; Z² is CH; Z³ is N; Z⁴ is N; m4 is 1; m5 is 0. In some embodiments, R^{a4} is methyl.

In some embodiments of Formula (I-a-2), the

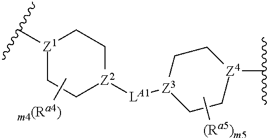

moiety is selected from the group consisting of the moieties delineated in Table L-I-a-2, wherein bb represents the point of attachment to Ring C.

TABLE L-I-a-2

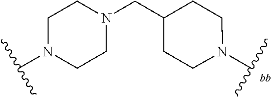 [SMILES: *N1CCC(CN2CCN(*)CC2)CC1]

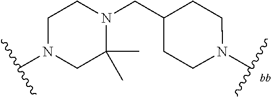 [SMILES: *N1CCC(CN2CCN(*)CC2(C)C)CC1]

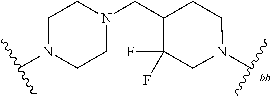 [SMILES: *N1CCN(CC2CCN(*)CC2(F)F)CC1]

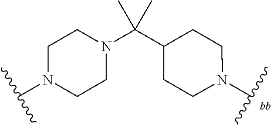 [SMILES: *N1CCC(C(C)(C)N2CCN(*)CC2)CC1]

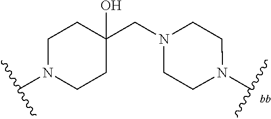 [SMILES: *N1CCN(CC2(O)CCN(*)CC2)CC1]

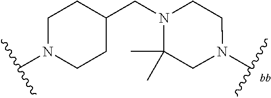 [SMILES: *N1CCC(CN2CCN(*)CC2(C)C)CC1]

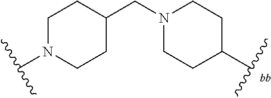 [SMILES: *C1CCN(CC2CCN(*)CC2)CC1]

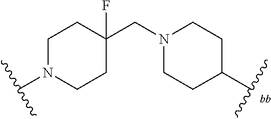 [SMILES: *C1CCN(CC2(F)CCN(*)CC2)CC1]

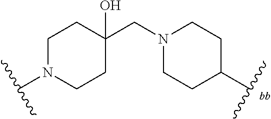 [SMILES: *C1CCN(CC2(O)CCN(*)CC2)CC1]

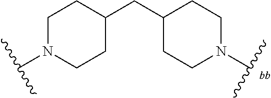 [SMILES: *N1CCC(CC2CCN(*)CC2)CC1]

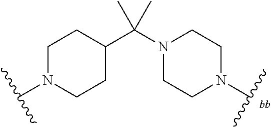 [SMILES: *N1CCC(C(C)(C)N2CCN(*)CC2)CC1]

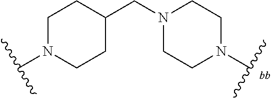 [SMILES: *N1CCC(CN2CCN(*)CC2)CC1]

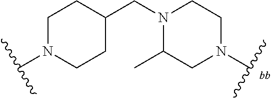 [SMILES: *N1CCC(CN2CCN(*)CC2C)CC1]

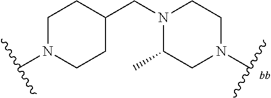 [SMILES: *N1CCC(CN2CCN(*)C[C@@H]2C)CC1]

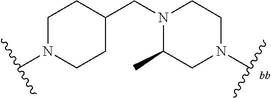 [SMILES: *N1CCC(CN2CCN(*)C[C@H]2C)CC1]

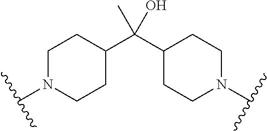 [SMILES: *N1CCC(C(C)(O)C2CCN(*)CC2)CC1]

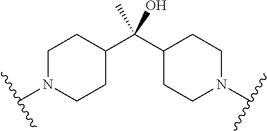

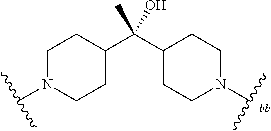

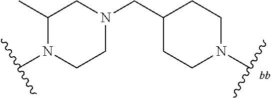 [SMILES: *N1CCC(CN2CCN(*)C(C)C2)CC1]

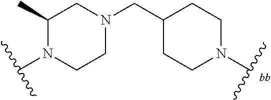 [SMILES: *N1CCC(CN2CCN(*)[C@@H](C)C2)CC1]

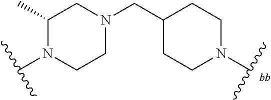 [SMILES: *N1CCC(CN2CCN(*)[C@H](C)C2)CC1]

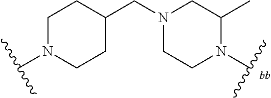 [SMILES: *N1CCC(CN2CCN(*)C(C)C2)CC1]

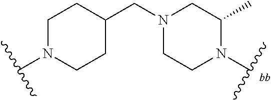 [SMILES: *N1CCC(CN2CCN(*)[C@@H](C)C2)CC1]

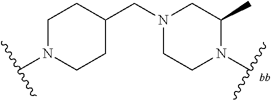 [SMILES: *N1CCC(CN2CCN(*)[C@H](C)C2)CC1]

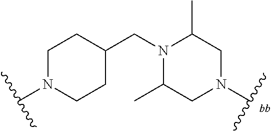 [SMILES: *N1CCC(CN2C(C)CN(*)CC2C)CC1]

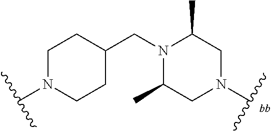 [SMILES: *N1CCC(CN2[C@H](C)CN(*)C[C@@H]2C)CC1]

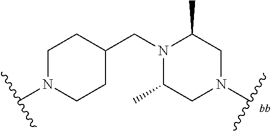 [SMILES: *N1CCC(CN2[C@@H](C)CN(*)C[C@@H]2C)CC1]

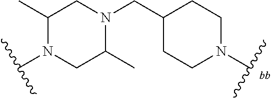 [SMILES: *N1CCC(CN2CC(C)N(*)CC2C)CC1]

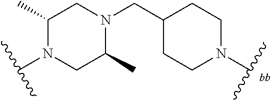 [SMILES: *N1CCC(CN2C[C@@H](C)N(*)C[C@@H]2C)CC1]

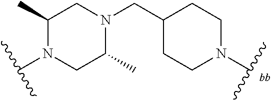 [SMILES: *N1CCC(CN2C[C@H](C)N(*)C[C@H]2C)CC1]

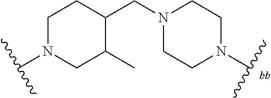 [SMILES: *N1CCN(CC2CCN(*)CC2C)CC1]

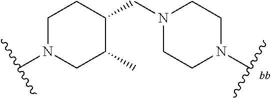 [SMILES: *N1CCN(C[C@H]2CCN(*)C[C@H]2C)CC1]

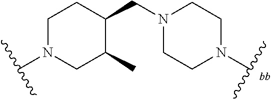 [SMILES: *N1CCN(C[C@@H]2CCN(*)C[C@@H]2C)CC1]

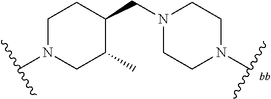 [SMILES: *N1CCN(C[C@@H]2CCN(*)C[C@H]2C)CC1]

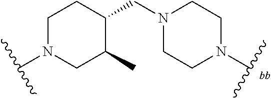 [SMILES: *N1CCN(C[C@H]2CCN(*)C[C@@H]2C)CC1]

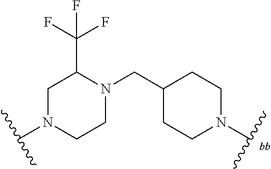 [SMILES: *N1CCC(CN2CCN(*)CC2C(F)(F)F)CC1]

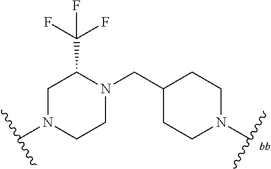 [SMILES: *N1CCC(CN2CCN(*)C[C@@H]2C(F)(F)F)CC1]

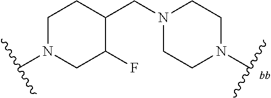 [SMILES: *N1CCN(CC2CCN(*)CC2F)CC1]

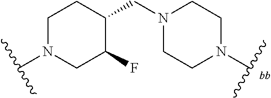 [SMILES: *N1CCN(C[C@H]2CCN(*)C[C@@H]2F)CC1]

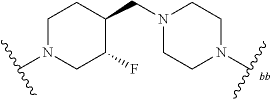 [SMILES: *N1CCN(C[C@@H]2CCN(*)C[C@H]2F)CC1]

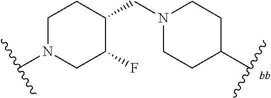 [SMILES: *C1CCN(C[C@H]2CCN(*)C[C@H]2F)CC1]

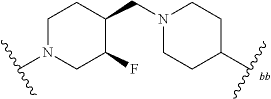 [SMILES: *C1CCN(C[C@@H]2CCN(*)C[C@@H]2F)CC1]

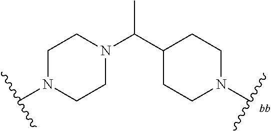 [SMILES: *N1CCC(C(C)N2CCN(*)CC2)CC1]

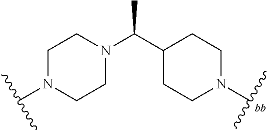 [SMILES: *N1CCC([C@H](C)N2CCN(*)CC2)CC1]

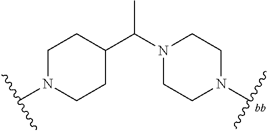 [SMILES: *N1CCC(C(C)N2CCN(*)CC2)CC1]

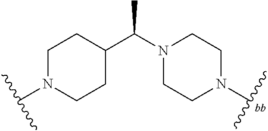 [SMILES: *N1CCC([C@@H](C)N2CCN(*)CC2)CC1]

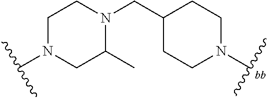 [SMILES: *N1CCC(CN2CCN(*)CC2C)CC1]

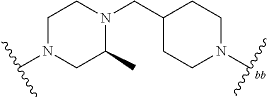 [SMILES: *N1CCC(CN2CCN(*)C[C@@H]2C)CC1]

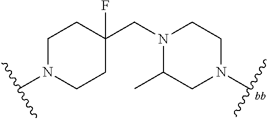 [SMILES: *N1CCC(F)(CN2CCN(*)CC2C)CC1]

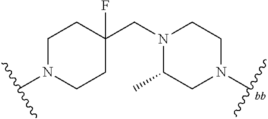 [SMILES: *N1CCC(F)(CN2CCN(*)C[C@@H]2C)CC1]

In some embodiments, the compounds of Formula (I-a) are compounds of Formula (I-a-3):

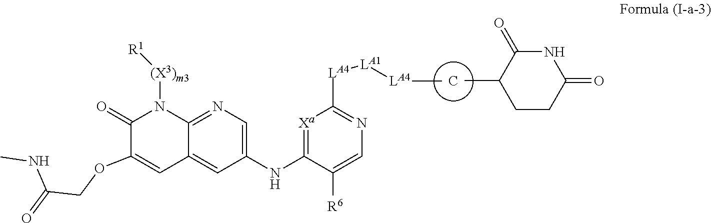

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

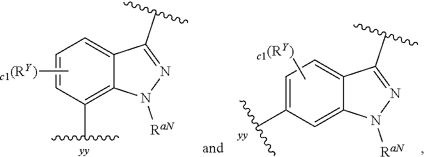

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂; and
one L^{A4} is a monocyclic 4-6 membered nitrogen-containing heterocyclylene optionally substituted with 1-3 R^{a}; and
the other L^{A4} is a bicyclic 6-12 membered nitrogen-containing heterocyclylene optionally substituted with 1-3 R^{a}, wherein:
each L^{A4} contains 1-2 ring nitrogen atoms and no additional ring heteroatom, and
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-a3), one L^{A4} is a monocyclic 4-6 membered nitrogen-containing heterocyclylene optionally substituted with 1-3 R^{a}; and the other L^{A4} is a bicyclic spirocyclic 6-12 (e.g., 7, 9, or 11) membered nitrogen-containing heterocyclylene optionally substituted with 1-3 R^{a}.

In some embodiments of Formula (I-a3), one L^{A4} is a monocyclic 4-6 membered nitrogen-containing heterocyclylene optionally substituted with 1-3 R^{a}; and the other L^{A4} is a bicyclic bridged 6-12 (e.g., 7, 8, or 9) membered nitrogen-containing heterocyclylene optionally substituted with 1-3 R^{a}

In some embodiments of Formula (I-a3), one L^{A4} is a monocyclic 4-6 membered nitrogen-containing heterocyclylene optionally substituted with 1-3 R^{a}; and the other L^{A4} is a bicyclic fused 6-12 (e.g., 6) membered nitrogen-containing heterocyclylene optionally substituted with 1-3 R^{a}.

In some embodiments of Formula (I-a-3), the -L^{A4}-L^{A1}_L^{A4}- moiety is selected from the group consisting of the moieties delineated in Table L-I-a-3, wherein bb represents the point of attachment to Ring C.

TABLE L-I-a-3

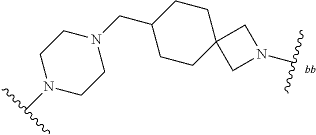 [SMILES: *N1CCN(CC2CCC3(CC2)CN(*)C3)CC1]

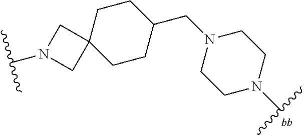 [SMILES: *N1CCN(CC2CCC3(CC2)CN(*)C3)CC1]

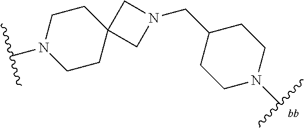 [SMILES: *N1CCC(CN2CC3(CCN(*)CC3)C2)CC1]

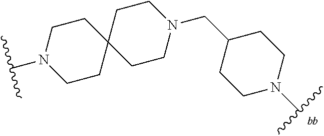 [SMILES: *N1CCC(CN2CCC3(CCN(*)CC3)CC2)CC1]

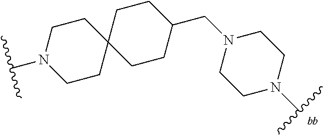 [SMILES: *N1CCN(CC2CCC3(CC2)CCN(*)CC3)CC1]

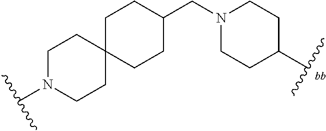 [SMILES: *C1CCN(CC2CCC3(CC2)CCN(*)CC3)CC1]

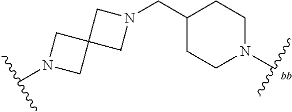 [SMILES: *N1CCC(CN2CC3(CN(*)C3)C2)CC1]

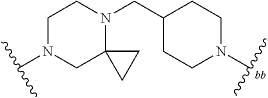 [SMILES: *N1CCC(CN2CCN(*)CC23CC3)CC1]

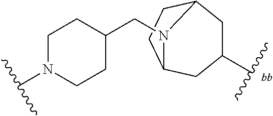 [SMILES: *C1CC2CCC(C1)N2CC1CCN(*)CC1]

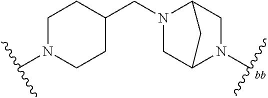 [SMILES: *N1CCC(CN2CC3CC2CN3*)CC1]

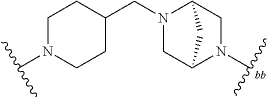 [SMILES: *N1CCC(CN2C[C@H]3C[C@@H]2CN3*)CC1]

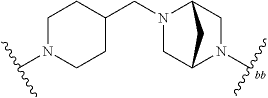 [SMILES: *N1CCC(CN2C[C@@H]3C[C@H]2CN3*)CC1]

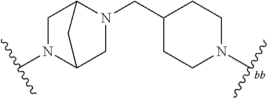 [SMILES: *N1CCC(CN2CC3CC2CN3*)CC1]

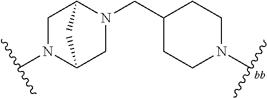 [SMILES: *N1CCC(CN2C[C@@H]3C[C@H]2CN3*)CC1]

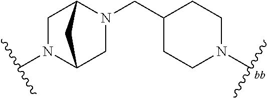 [SMILES: *N1CCC(CN2C[C@H]3C[C@@H]2CN3*)CC1]

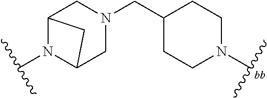 [SMILES: *N1CCC(CN2CC3CC(C2)N3*)CC1]

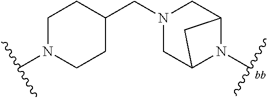 [SMILES: *N1CCC(CN2CC3CC(C2)N3*)CC1]

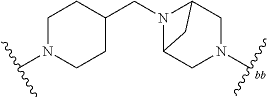 [SMILES: *N1CCC(CN2C3CC2CN(*)C3)CC1]

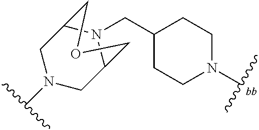 [SMILES: *N1CCC(CN2C3COCC2CN(*)C3)CC1]

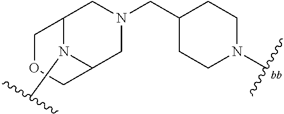 [SMILES: *N1CCC(CN2CC3COCC(C2)N3*)CC1]

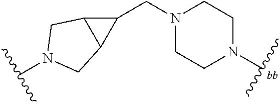 [SMILES: *N1CCN(CC2C3CN(*)CC32)CC1]

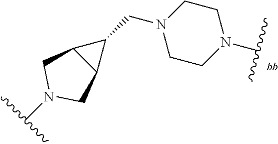 [SMILES: *N1CCN(C[C@H]2[C@@H]3CN(*)C[C@@H]32)CC1]

In some embodiments, the compounds of Formula (I-a) are compounds of Formula (I-a-4):

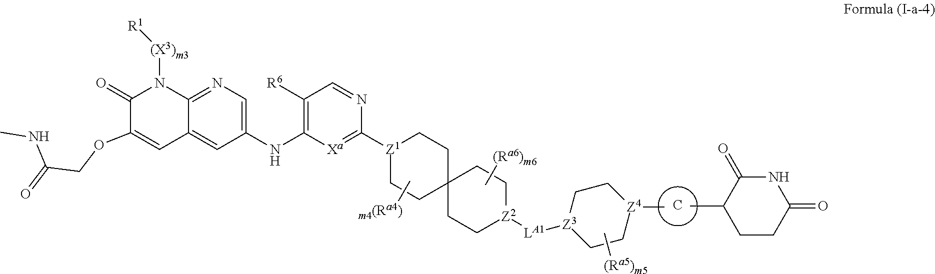

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

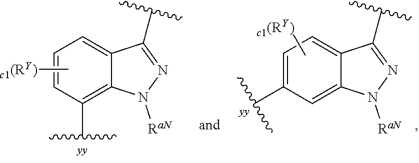

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂;
Z¹ and Z² are independently selected from the group consisting of: CH, CR^{a4}, and N;
Z³ and Z⁴ are independently selected from the group consisting of: CH, CR^{a5}, and N,
provided that at least one of Z¹ and Z² is N; at least one of Z³ and ZA is N; and when Z² is N, then Z³ is CH or CR^{a5},
m4 and m6 are independently 0 or 1;
m5 is 0, 1, or 2; and
each R^{a4}, R^{a5}, and R^{a6} is independently selected from the group consisting of: —F, CN, C₁₋₃ alkoxy, OH, and C₁₋₃ alkyl optionally substituted with 1-3 F.

In some embodiments of Formula (I-a4), Z¹ is N. In some embodiments of Formula (I-a4), Z¹ is N; and Z² is N. In some embodiments of Formula (I-a4), Z¹ is N; Z² is N; Z³ is CH; and Z⁴ is N. In some embodiments of Formula (I-a4), Z¹ is N; Z² is N; Z³ is CH; Z⁴ is N; and 2-3 (e.g., 3) of m4, m6, and m6 are 0.

In some embodiments of Formula (I-a-4), the

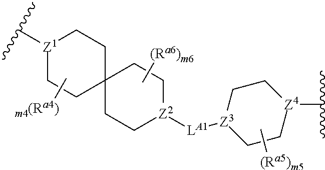

moiety is selected from the group consisting of the moieties delineated in Table L-I-a-4, wherein bb represents the point of attachment to Ring C.

TABLE L-I-a-4

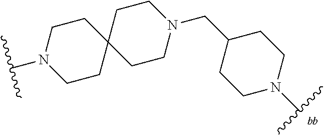 [SMILES: *N1CCC(CN2CCC3(CCN(*)CC3)CC2)CC1]

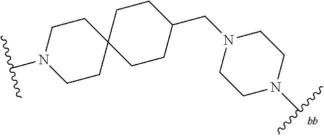 [SMILES: *N1CCN(CC2CCC3(CC2)CCN(*)CC3)CC1]

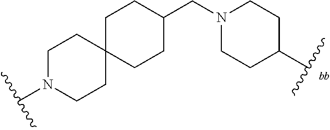 [SMILES: *C1CCN(CC2CCC3(CC2)CCN(*)CC3)CC1]

In some embodiments, the compounds of Formula (I-a) are compounds of Formula (I-a-5):

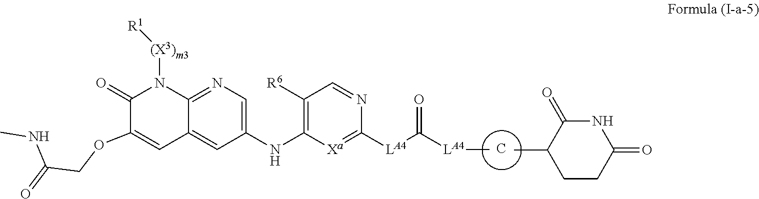

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

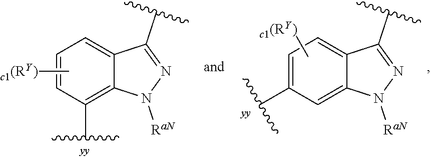

wherein: c1 is 0 or 1, R^{Y} is selected from the group consisting of halo (e.g., —F) and C₁₋₃ alkyl optionally substituted with 1-3 F, and R^{aN} is C₁₋₃ alkyl; and
each L^{A4} is independently 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-a-5), each L^{A4} is independently monocyclic 4-6 membered nitrogen-containing heterocyclylene optionally substituted with 1-3 R^{a}, wherein each L^{A4} contains 1-2 ring nitrogen atoms and no additional ring heteroatoms. For example, each L^{A4} can be independently selected from the group consisting of: piperazinylene and piperidinylene.

In some embodiments of Formula (I-a-5), the -L^{A4}-C(—O)L^{A4}- moiety is selected from the group consisting of the moieties delineated in Table L-I-a-5, wherein bb represents the point of attachment to Ring C.

TABLE L-I-a-5

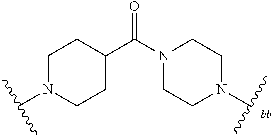 [SMILES: *N1CCC(C(=O)N2CCN(*)CC2)CC1]

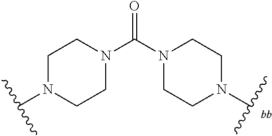 [SMILES: *N1CCN(C(=O)N2CCN(*)CC2)CC1]

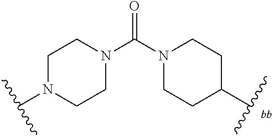 [SMILES: *C1CCN(C(=O)N2CCN(*)CC2)CC1]

In some embodiments, the compounds of Formula (I-a) are compounds of Formula (I-a-6):

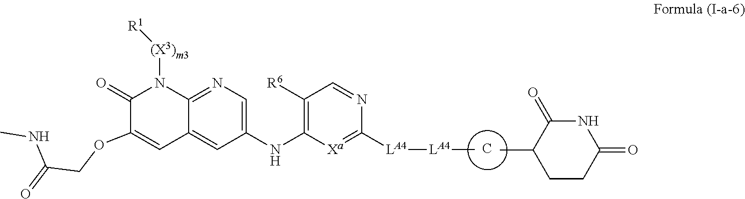

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

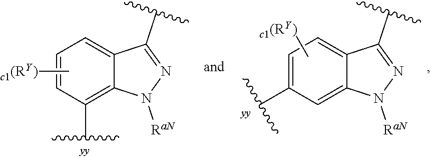

wherein: c1 is 0 or 1, R^{Y} is selected from the group consisting of halo (e.g., —F) and C₁₋₃ alkyl optionally substituted with 1-3 F, and R^{aN} is C₁₋₃ alkyl; and
each L^{A4} is independently 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-a-6), each L^{A4} is independently monocyclic 4-6 membered nitrogen-containing heterocyclylene optionally substituted with 1-3 R^{a}, wherein each L^{A4} contains 1-2 ring nitrogen atoms and no additional ring heteroatoms. For example, each L^{A4} can be independently selected from the group consisting of: piperazinylene, piperidinylene, and pyrrolidinylene.

In some embodiments of Formula (I-a), (I-a-1), (I-a-2), (I-a-3), (I-a-4), (I-a-5), or (I-a-6), Ring C is selected from the group consisting of:

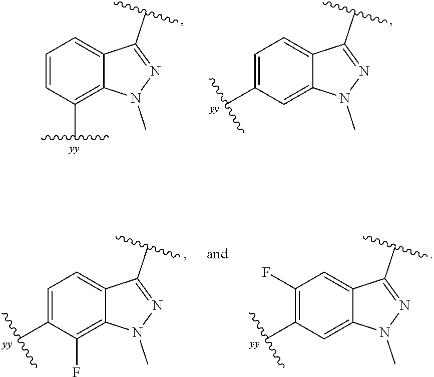 [SMILES: *c1nn(C)c2c(*)cccc12 ; *c1ccc2c(*)nn(C)c2c1 ; *c1ccc2c(*)nn(C)c2c1F ; *c1cc2c(cc1F)c(*)nn2C]

For example, Ring C can be

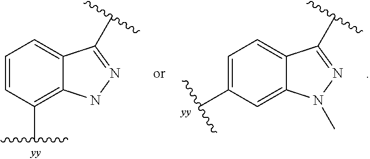 [SMILES: *c1n[nH]c2c(*)cccc12 ; *c1ccc2c(*)nn(C)c2c1]

For example, Ring C can be

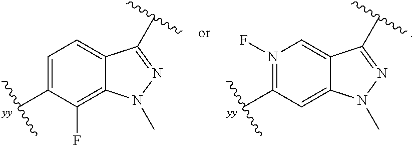

In some embodiments of Formula (I-a), (I-a-1), (I-a-2), (I-a-3), (I-a-4), (I-a-5), or (I-a-6), X^{a} is N.

In some embodiments of Formula (I-a), (I-a-1), (I-a-2), (I-a-3), (I-a-4), (I-a-5), or (I-a-6), X^{a} is CH.

In some embodiments, the compounds of Formula (I) are compounds of Formula (I-b):

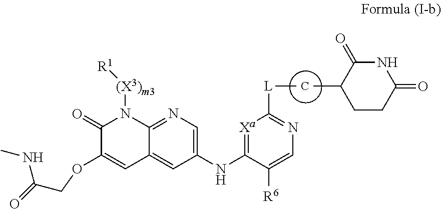

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

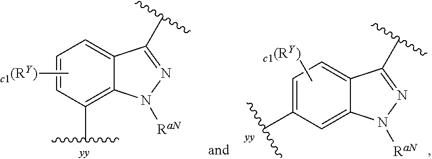

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L is -L^{A4}-L^{A3}-_{bb} or -L^{A4}-L^{A1}_L^{A3}-_{bb}, wherein bb represents the point of attachment to Ring C; and
L^{A4} is 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-b), L is -L^{A4}-L^{A3}-_{bb}, and L^{A3} is —NH—.

In some embodiments of Formula (I-b), L^{A4} is a monocyclic 4-6 membered nitrogen-containing heterocyclylene optionally substituted with 1-3 R^{a}, wherein L^{A4} contains 1-2 ring nitrogen atoms and no additional ring heteroatoms.

In some embodiments of Formula (I-b), L^{A4} is a bicyclic spirocyclic 6-12 (e.g., 6-10) membered nitrogen-containing heterocyclylene optionally substituted with 1-3 R^{a}, wherein L^{A4} contains 1-2 ring nitrogen atoms and no additional ring heteroatoms.

In some embodiments of Formula (I-b), X^{a} is N.

In some embodiments of Formula (I-b), X^{a} is CH.

In some embodiments of Formula (I-b), L is selected from the group consisting of the moieties delineated in Table L-I-b, wherein bb represents the point of attachment to Ring C.

TABLE L-I-b

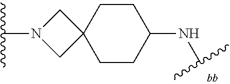 [SMILES: *NC1CCC2(CC1)CN(*)C2]

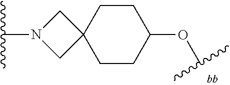 [SMILES: *OC1CCC2(CC1)CN(*)C2]

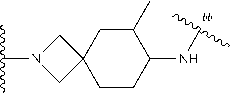 [SMILES: *NC1CCC2(CC1C)CN(*)C2]

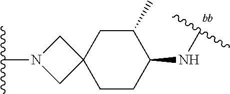 [SMILES: *N[C@H]1CCC2(C[C@@H]1C)CN(*)C2]

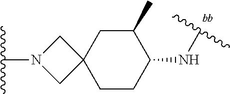 [SMILES: *N[C@@H]1CCC2(C[C@H]1C)CN(*)C2]

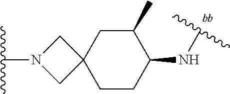 [SMILES: *N[C@H]1CCC2(C[C@H]1C)CN(*)C2]

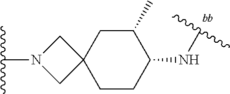 [SMILES: *N[C@@H]1CCC2(C[C@@H]1C)CN(*)C2]

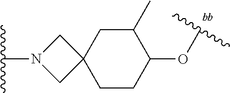 [SMILES: *OC1CCC2(CC1C)CN(*)C2]

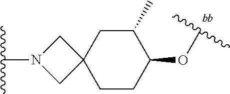 [SMILES: *O[C@H]1CCC2(C[C@@H]1C)CN(*)C2]

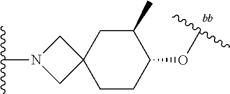 [SMILES: *O[C@@H]1CCC2(C[C@H]1C)CN(*)C2]

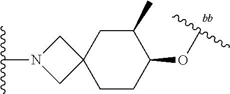 [SMILES: *O[C@H]1CCC2(C[C@H]1C)CN(*)C2]

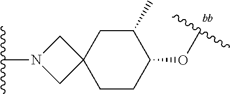 [SMILES: *O[C@@H]1CCC2(C[C@@H]1C)CN(*)C2]

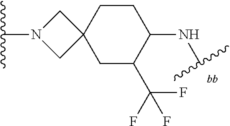 [SMILES: *NC1CCC2(CC1C(F)(F)F)CN(*)C2]

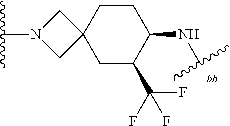 [SMILES: *N[C@@H]1CCC2(C[C@@H]1C(F)(F)F)CN(*)C2]

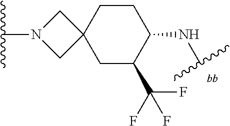 [SMILES: *N[C@H]1CCC2(C[C@@H]1C(F)(F)F)CN(*)C2]

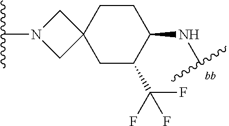 [SMILES: *N[C@@H]1CCC2(C[C@H]1C(F)(F)F)CN(*)C2]

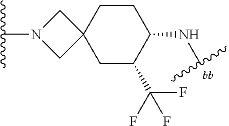 [SMILES: *N[C@H]1CCC2(C[C@H]1C(F)(F)F)CN(*)C2]

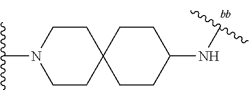 [SMILES: *NC1CCC2(CC1)CCN(*)CC2]

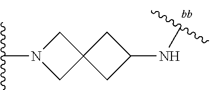 [SMILES: *NC1CC2(C1)CN(*)C2]

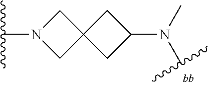 [SMILES: *N1CC2(CC(N(*)C)C2)C1]

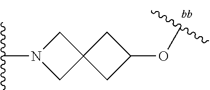 [SMILES: *OC1CC2(C1)CN(*)C2]

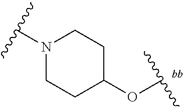 [SMILES: *OC1CCN(*)CC1]

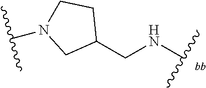 [SMILES: *NCC1CCN(*)C1]

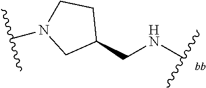 [SMILES: *NC[C@@H]1CCN(*)C1]

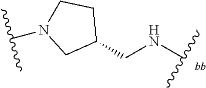 [SMILES: *NC[C@H]1CCN(*)C1]

In some embodiments, the compounds of Formula (I-b) are compounds of Formula (I-b-1):

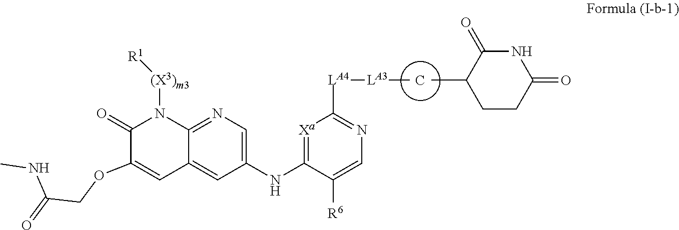

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

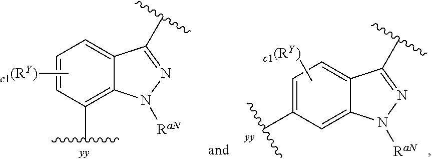

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;

L^{A4} is 6-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F; and
L^{A3} is —NH—, —N(C₁₋₃ alkyl)-, or —O—.

In some embodiments of Formula (I-b-1), L^{A4} is a bicyclic spirocyclic 6-12 (e.g., 8-10 (e.g., 9)) membered nitrogen-containing heterocyclylene optionally substituted with 1-3 R^{a}, wherein L^{A4} contains 1-2 (e.g., 1) ring nitrogen atoms and no additional ring heteroatoms.

In some embodiments of Formula (I-b-1), the -L^{A4}-L^{A3}- moiety is selected from the group consisting of the moieties delineated in Table L-I-b-1, wherein bb represents the point of attachment to Ring C.

TABLE L-I-b-1

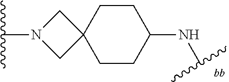 [SMILES: *NC1CCC2(CC1)CN(*)C2]

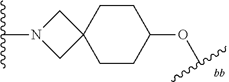 [SMILES: *OC1CCC2(CC1)CN(*)C2]

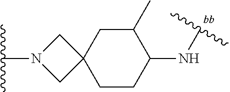 [SMILES: *NC1CCC2(CC1C)CN(*)C2]

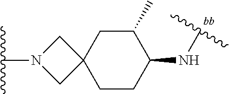 [SMILES: *N[C@H]1CCC2(C[C@@H]1C)CN(*)C2]

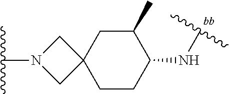 [SMILES: *N[C@@H]1CCC2(C[C@H]1C)CN(*)C2]

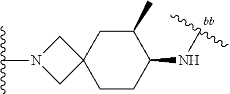 [SMILES: *N[C@H]1CCC2(C[C@H]1C)CN(*)C2]

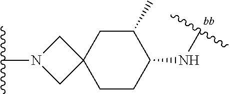 [SMILES: *N[C@@H]1CCC2(C[C@@H]1C)CN(*)C2]

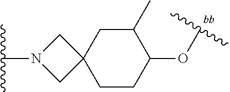 [SMILES: *OC1CCC2(CC1C)CN(*)C2]

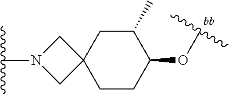 [SMILES: *O[C@H]1CCC2(C[C@@H]1C)CN(*)C2]

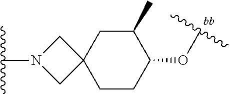 [SMILES: *O[C@@H]1CCC2(C[C@H]1C)CN(*)C2]

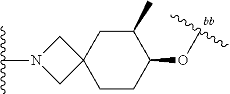 [SMILES: *O[C@H]1CCC2(C[C@H]1C)CN(*)C2]

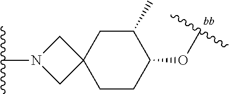 [SMILES: *O[C@@H]1CCC2(C[C@@H]1C)CN(*)C2]

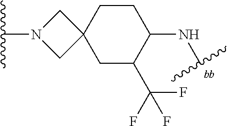 [SMILES: *NC1CCC2(CC1C(F)(F)F)CN(*)C2]

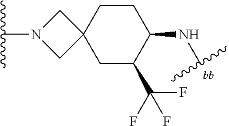 [SMILES: *N[C@@H]1CCC2(C[C@@H]1C(F)(F)F)CN(*)C2]

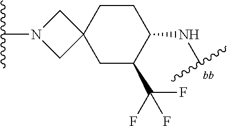 [SMILES: *N[C@H]1CCC2(C[C@@H]1C(F)(F)F)CN(*)C2]

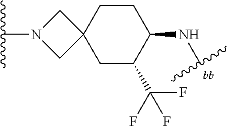 [SMILES: *N[C@@H]1CCC2(C[C@H]1C(F)(F)F)CN(*)C2]

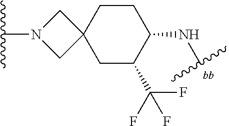 [SMILES: *N[C@H]1CCC2(C[C@H]1C(F)(F)F)CN(*)C2]

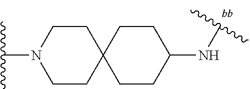 [SMILES: *NC1CCC2(CC1)CCN(*)CC2]

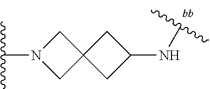 [SMILES: *NC1CC2(C1)CN(*)C2]

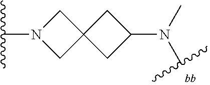 [SMILES: *N1CC2(CC(N(*)C)C2)C1]

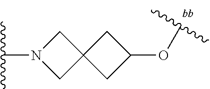 [SMILES: *OC1CC2(C1)CN(*)C2]

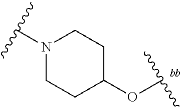 [SMILES: *OC1CCN(*)CC1]

In some embodiments, the compounds of Formula (I-b) are compounds of Formula (I-b-2):

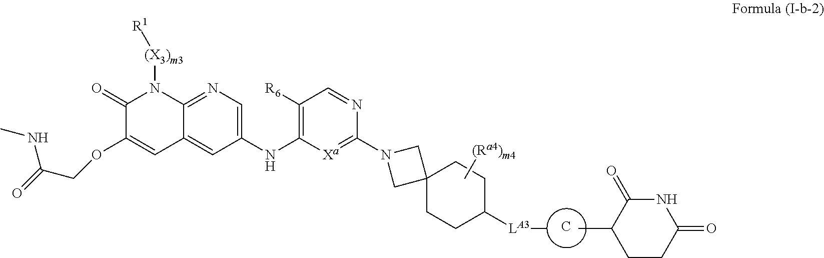

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

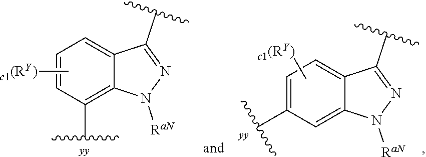

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A3} is —NH—, —N(C₁₋₃ alkyl)-, or —O—;
m4 is selected from the group consisting of: 0, 1, and 2; and
each R^{a4} is independently selected from the group consisting of: —F, CN, C₁₋₃ alkoxy, OH, and C₁₋₃ alkyl optionally substituted with 1-3 F.

In some embodiments of Formula (I-b-2), m4 is 0 or 1; and R^{a4} when present is methyl.

In some embodiments of Formula (I-b-2), the

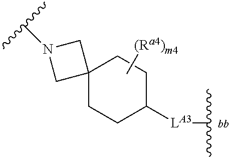

moiety is selected from the group consisting of the moieties delineated in Table L-I-b-2, wherein bb represents the point of attachment to Ring C.

TABLE L-I-b-2

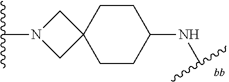 [SMILES: *NC1CCC2(CC1)CN(*)C2]

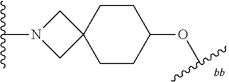 [SMILES: *OC1CCC2(CC1)CN(*)C2]

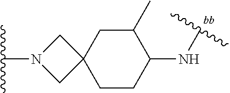 [SMILES: *NC1CCC2(CC1C)CN(*)C2]

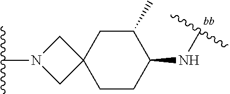 [SMILES: *N[C@H]1CCC2(C[C@@H]1C)CN(*)C2]

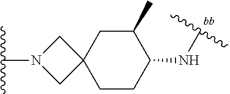 [SMILES: *N[C@@H]1CCC2(C[C@H]1C)CN(*)C2]

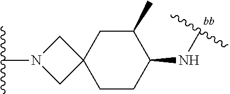 [SMILES: *N[C@H]1CCC2(C[C@H]1C)CN(*)C2]

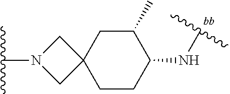 [SMILES: *N[C@@H]1CCC2(C[C@@H]1C)CN(*)C2]

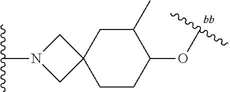 [SMILES: *OC1CCC2(CC1C)CN(*)C2]

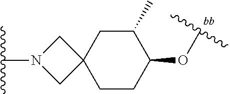 [SMILES: *O[C@H]1CCC2(C[C@@H]1C)CN(*)C2]

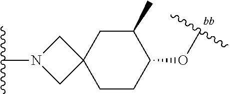 [SMILES: *O[C@@H]1CCC2(C[C@H]1C)CN(*)C2]

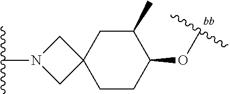 [SMILES: *O[C@H]1CCC2(C[C@H]1C)CN(*)C2]

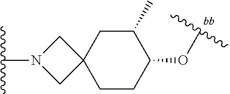 [SMILES: *O[C@@H]1CCC2(C[C@@H]1C)CN(*)C2]

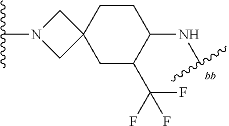 [SMILES: *NC1CCC2(CC1C(F)(F)F)CN(*)C2]

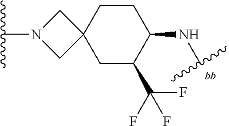 [SMILES: *N[C@@H]1CCC2(C[C@@H]1C(F)(F)F)CN(*)C2]

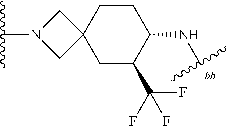 [SMILES: *N[C@H]1CCC2(C[C@@H]1C(F)(F)F)CN(*)C2]

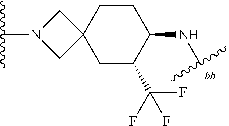 [SMILES: *N[C@@H]1CCC2(C[C@H]1C(F)(F)F)CN(*)C2]

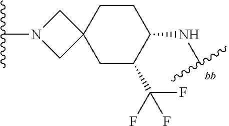 [SMILES: *N[C@H]1CCC2(C[C@H]1C(F)(F)F)CN(*)C2]

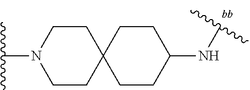 [SMILES: *NC1CCC2(CC1)CCN(*)CC2]

In some embodiments, the compounds are selected from the group consisting of the compounds in Table C1, or pharmaceutically acceptable salts thereof.

TABLE C1

| No. | Compound Structure |
|---|---|
| 101 | 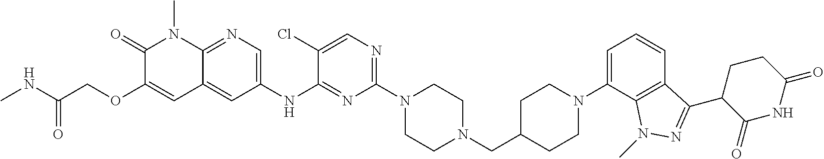 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)cnc2n(C)c1=O] |
| 102 | 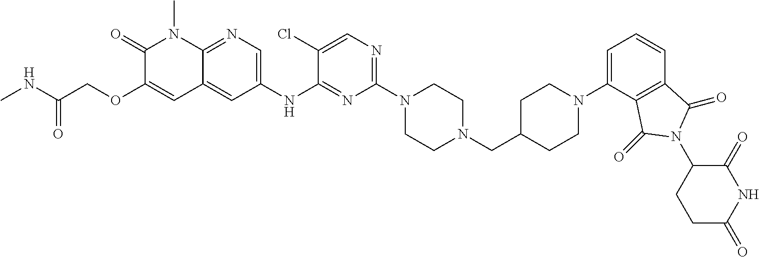 [SMILES: CNC(=O)COc1cc2cc(Nc3nc(N4CCN(CC5CCN(c6cccc7c6C(=O)N(C6CCC(=O)NC6=O)C7=O)CC5)CC4)ncc3Cl)cnc2n(C)c1=O] |
| 103 | 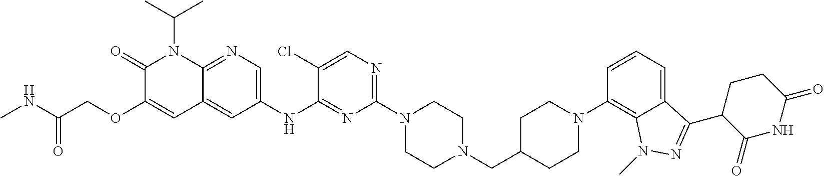 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)cnc2n(C(C)C)c1=O] |
| 104 | 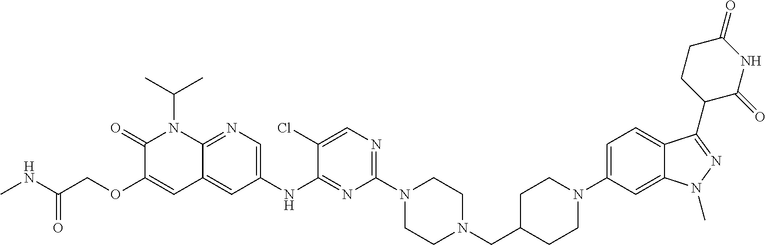 [SMILES: CNC(=O)COc1cc2cc(Nc3nc(N4CCN(CC5CCN(c6ccc7c(C8CCC(=O)NC8=O)nn(C)c7c6)CC5)CC4)ncc3Cl)cnc2n(C(C)C)c1=O] |
| 105 | 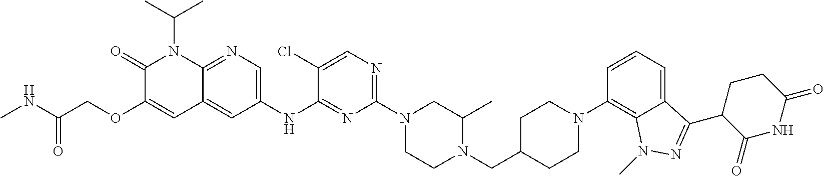 [SMILES: CNC(=O)COc1cc2cc(Nc3nc(N4CCN(CC5CCN(c6cccc7c(C8CCC(=O)NC8=O)nn(C)c67)CC5)C(C)C4)ncc3Cl)cnc2n(C(C)C)c1=O] |
| 105a | 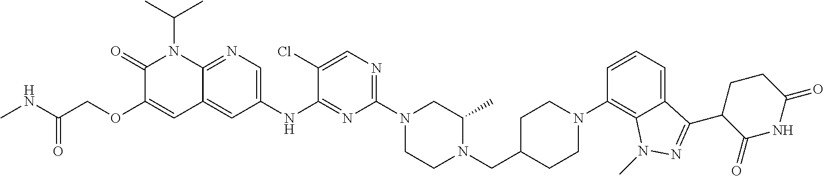 [SMILES: CNC(=O)COc1cc2cc(Nc3nc(N4CCN(CC5CCN(c6cccc7c(C8CCC(=O)NC8=O)nn(C)c67)CC5)[C@@H](C)C4)ncc3Cl)cnc2n(C(C)C)c1=O] |
| 106 | 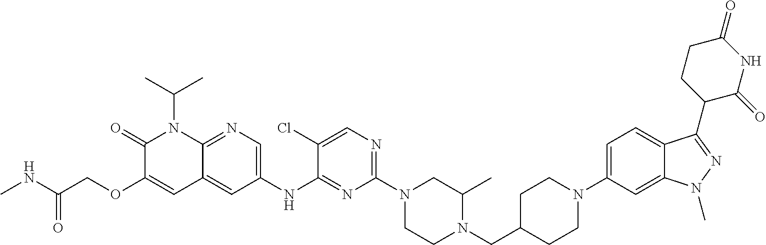 [SMILES: CNC(=O)COc1cc2cc(Nc3nc(N4CCN(CC5CCN(c6ccc7c(C8CCC(=O)NC8=O)nn(C)c7c6)CC5)C(C)C4)ncc3Cl)cnc2n(C(C)C)c1=O] |
| 106a | 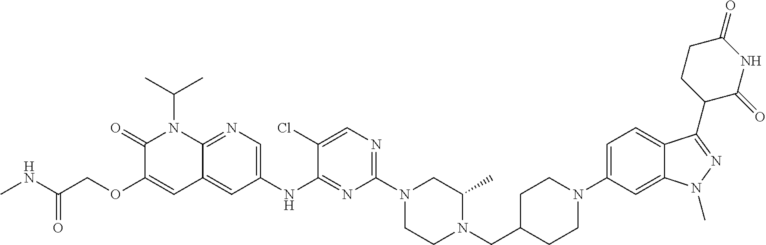 [SMILES: CNC(=O)COc1cc2cc(Nc3nc(N4CCN(CC5CCN(c6ccc7c(C8CCC(=O)NC8=O)nn(C)c7c6)CC5)[C@@H](C)C4)ncc3Cl)cnc2n(C(C)C)c1=O] |
| 107 | 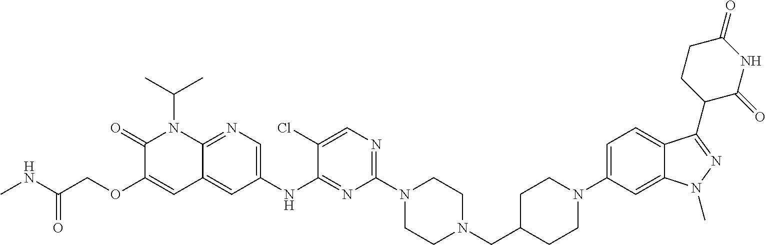 [SMILES: CNC(=O)COc1cc2cc(Nc3nc(N4CCN(CC5CCN(c6ccc7c(C8CCC(=O)NC8=O)nn(C)c7c6)CC5)CC4)ncc3Cl)cnc2n(C(C)C)c1=O] |
| 108 | 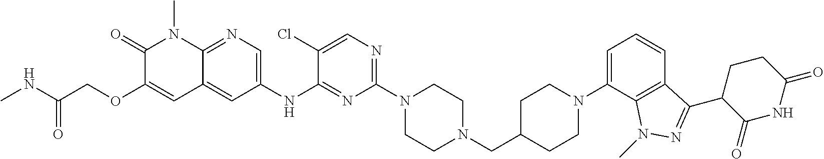 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)cnc2n(C)c1=O] |
| 109 | 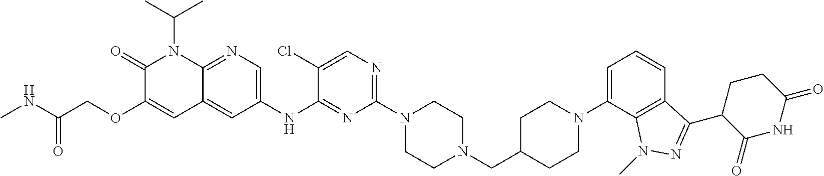 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)cnc2n(C(C)C)c1=O] |
| 110 | 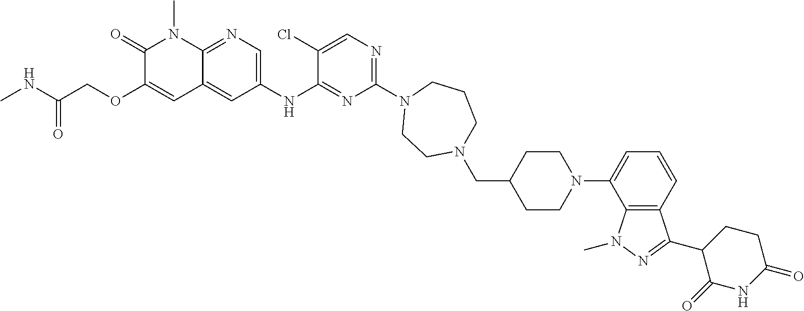 [SMILES: CNC(=O)COc1cc2cc(Nc3nc(N4CCCN(CC5CCN(c6cccc7c(C8CCC(=O)NC8=O)nn(C)c67)CC5)CC4)ncc3Cl)cnc2n(C)c1=O] |
| 111 | 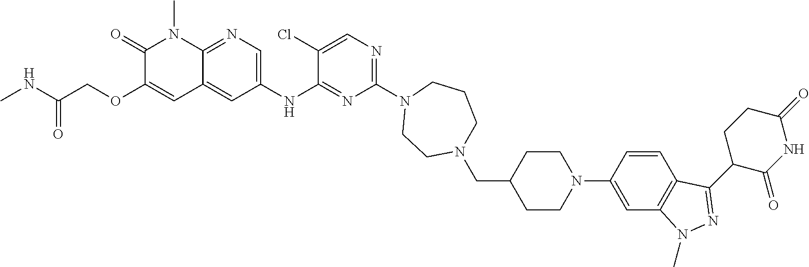 [SMILES: CNC(=O)COc1cc2cc(Nc3nc(N4CCCN(CC5CCN(c6ccc7c(C8CCC(=O)NC8=O)nn(C)c7c6)CC5)CC4)ncc3Cl)cnc2n(C)c1=O] |
| 112 | 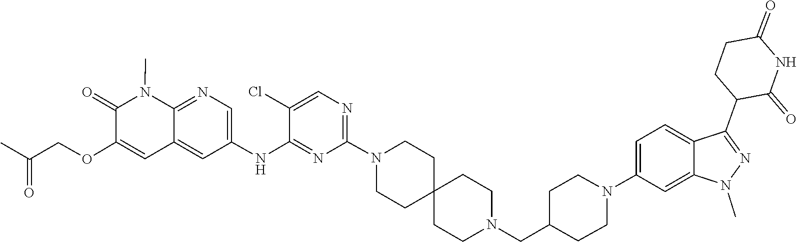 [SMILES: CC(=O)COc1cc2cc(Nc3nc(N4CCC5(CCN(CC6CCN(c7ccc8c(C9CCC(=O)NC9=O)nn(C)c8c7)CC6)CC5)CC4)ncc3Cl)cnc2n(C)c1=O] |
| 113 | 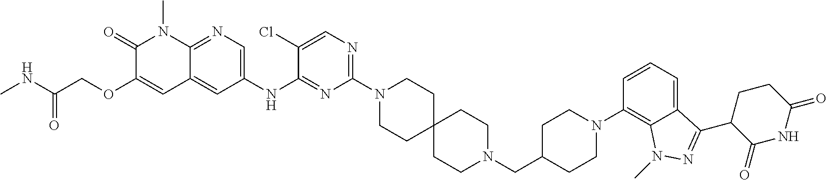 [SMILES: CNC(=O)COc1cc2cc(Nc3nc(N4CCC5(CCN(CC6CCN(c7cccc8c(C9CCC(=O)NC9=O)nn(C)c78)CC6)CC5)CC4)ncc3Cl)cnc2n(C)c1=O] |
| 114 | 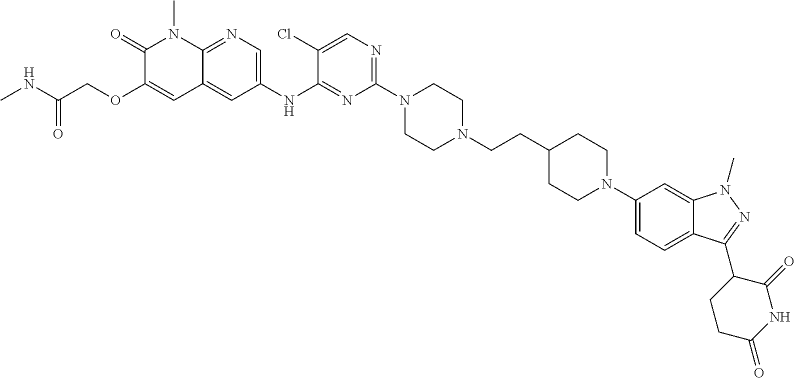 [SMILES: CNC(=O)COc1cc2cc(Nc3nc(N4CCN(CCC5CCN(c6ccc7c(C8CCC(=O)NC8=O)nn(C)c7c6)CC5)CC4)ncc3Cl)cnc2n(C)c1=O] |
| 115 | 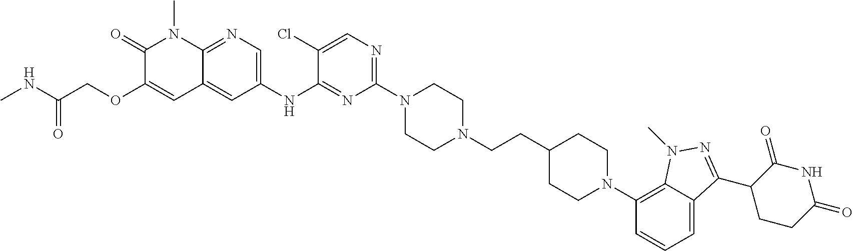 [SMILES: CNC(=O)COc1cc2cc(Nc3nc(N4CCN(CCC5CCN(c6cccc7c(C8CCC(=O)NC8=O)nn(C)c67)CC5)CC4)ncc3Cl)cnc2n(C)c1=O] |
| 116 | 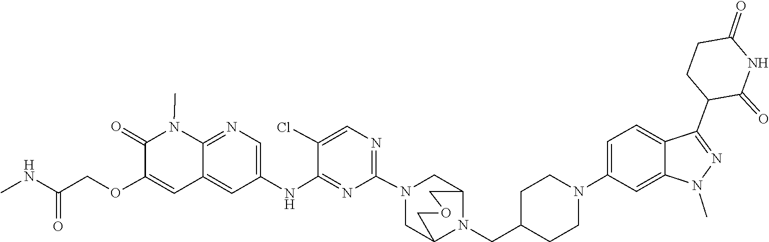 [SMILES: CNC(=O)COc1cc2cc(Nc3nc(N4CC5COCC(C4)N5CC4CCN(c5ccc6c(C7CCC(=O)NC7=O)nn(C)c6c5)CC4)ncc3Cl)cnc2n(C)c1=O] |
| 117 | 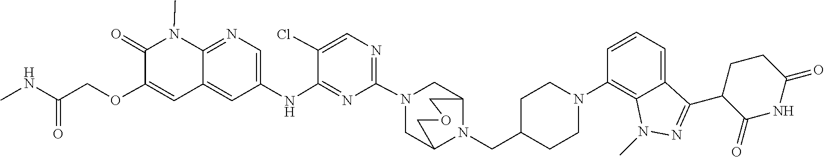 [SMILES: CNC(=O)COc1cc2cc(Nc3nc(N4CC5COCC(C4)N5CC4CCN(c5cccc6c(C7CCC(=O)NC7=O)nn(C)c56)CC4)ncc3Cl)cnc2n(C)c1=O] |
| 118 | 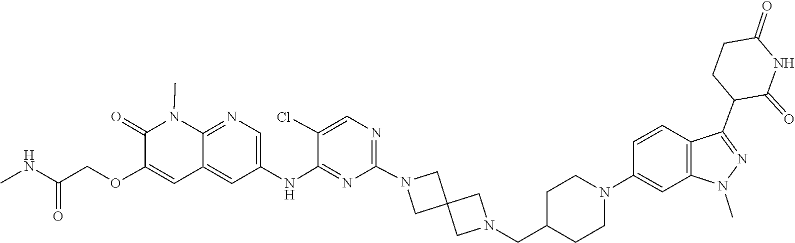 [SMILES: CNC(=O)COc1cc2cc(Nc3nc(N4CC5(CN(CC6CCN(c7ccc8c(C9CCC(=O)NC9=O)nn(C)c8c7)CC6)C5)C4)ncc3Cl)cnc2n(C)c1=O] |
| 119 | 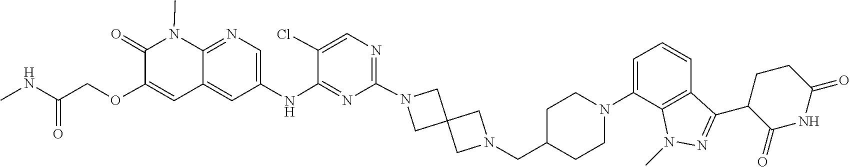 [SMILES: CNC(=O)COc1cc2cc(Nc3nc(N4CC5(CN(CC6CCN(c7cccc8c(C9CCC(=O)NC9=O)nn(C)c78)CC6)C5)C4)ncc3Cl)cnc2n(C)c1=O] |
| 120 | 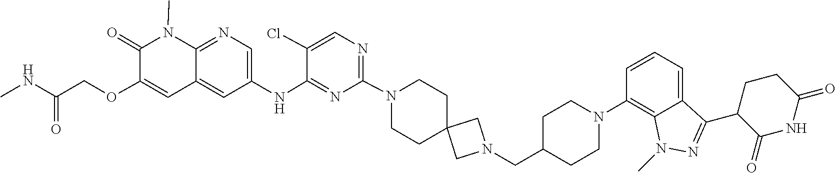 [SMILES: CNC(=O)COc1cc2cc(Nc3nc(N4CCC5(CC4)CN(CC4CCN(c6cccc7c(C8CCC(=O)NC8=O)nn(C)c67)CC4)C5)ncc3Cl)cnc2n(C)c1=O] |
| 121 | 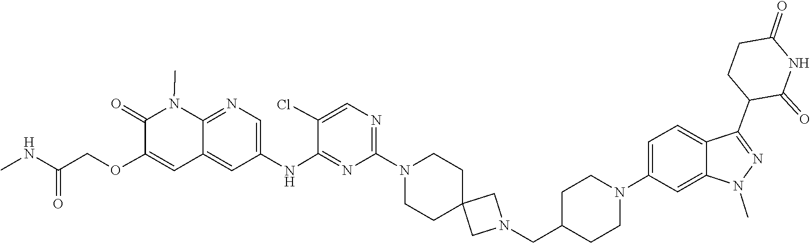 [SMILES: CNC(=O)COc1cc2cc(Nc3nc(N4CCC5(CC4)CN(CC4CCN(c6ccc7c(C8CCC(=O)NC8=O)nn(C)c7c6)CC4)C5)ncc3Cl)cnc2n(C)c1=O] |
| 122 | 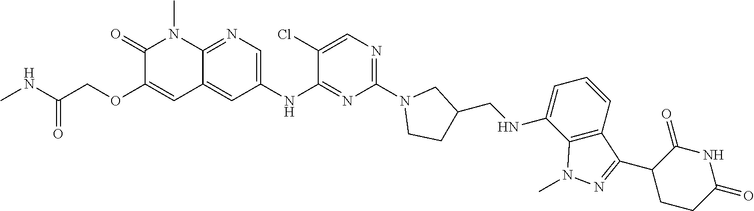 [SMILES: CNC(=O)COc1cc2cc(Nc3nc(N4CCC(CNc5cccc6c(C7CCC(=O)NC7=O)nn(C)c56)C4)ncc3Cl)cnc2n(C)c1=O] |
| 122a | 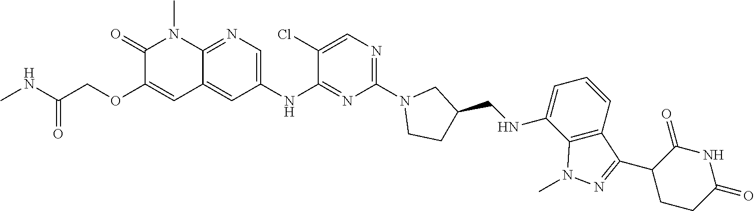 [SMILES: CNC(=O)COc1cc2cc(Nc3nc(N4CC[C@H](CNc5cccc6c(C7CCC(=O)NC7=O)nn(C)c56)C4)ncc3Cl)cnc2n(C)c1=O] |
| 122b | 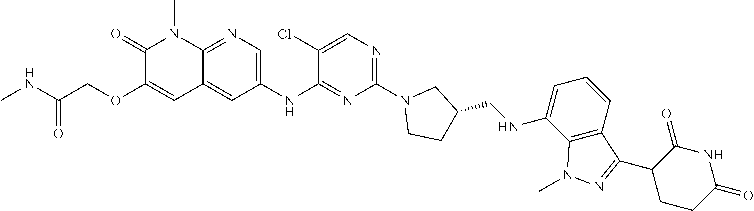 [SMILES: CNC(=O)COc1cc2cc(Nc3nc(N4CC[C@@H](CNc5cccc6c(C7CCC(=O)NC7=O)nn(C)c56)C4)ncc3Cl)cnc2n(C)c1=O] |
| 123 | 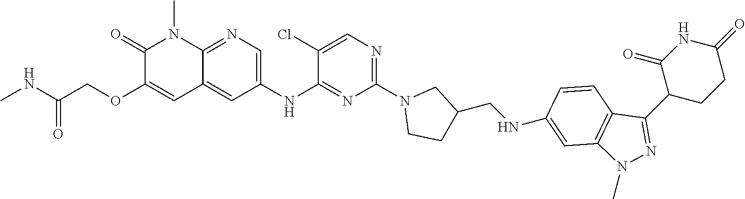 [SMILES: CNC(=O)COc1cc2cc(Nc3nc(N4CCC(CNc5ccc6c(C7CCC(=O)NC7=O)nn(C)c6c5)C4)ncc3Cl)cnc2n(C)c1=O] |
| 123a | 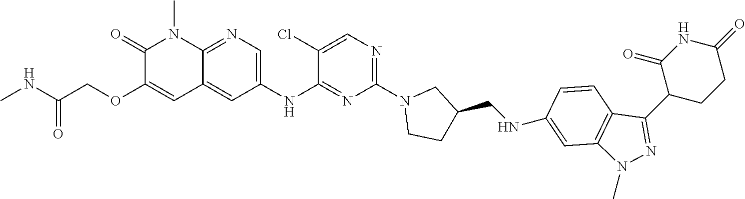 [SMILES: CNC(=O)COc1cc2cc(Nc3nc(N4CC[C@H](CNc5ccc6c(C7CCC(=O)NC7=O)nn(C)c6c5)C4)ncc3Cl)cnc2n(C)c1=O] |
| 123b | 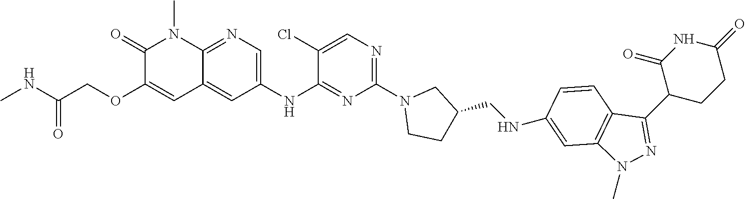 [SMILES: CNC(=O)COc1cc2cc(Nc3nc(N4CC[C@@H](CNc5ccc6c(C7CCC(=O)NC7=O)nn(C)c6c5)C4)ncc3Cl)cnc2n(C)c1=O] |
| 124 | 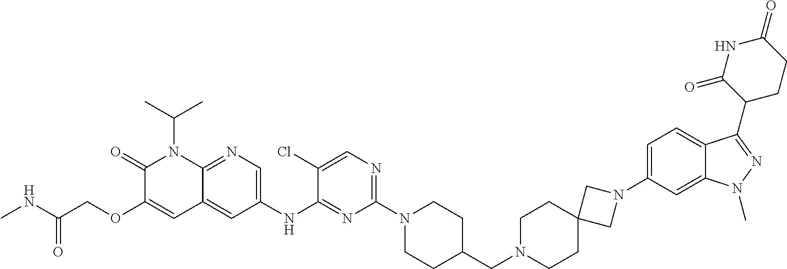 [SMILES: CNC(=O)COc1cc2cc(Nc3nc(N4CCC(CN5CCC6(CC5)CN(c5ccc7c(C8CCC(=O)NC8=O)nn(C)c7c5)C6)CC4)ncc3Cl)cnc2n(C(C)C)c1=O] |
| 125 | 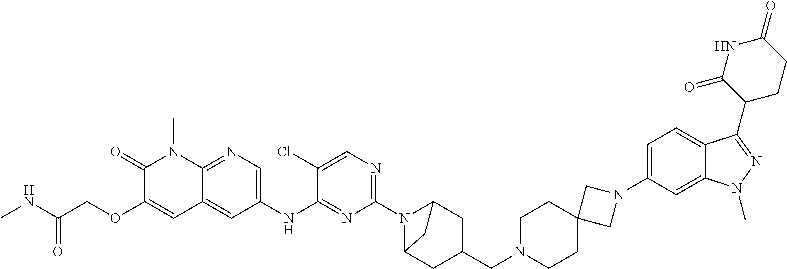 [SMILES: CNC(=O)COc1cc2cc(Nc3nc(N4C5CC(CN6CCC7(CC6)CN(c6ccc8c(C9CCC(=O)NC9=O)nn(C)c8c6)C7)CC4C5)ncc3Cl)cnc2n(C)c1=O] |
| 126 | 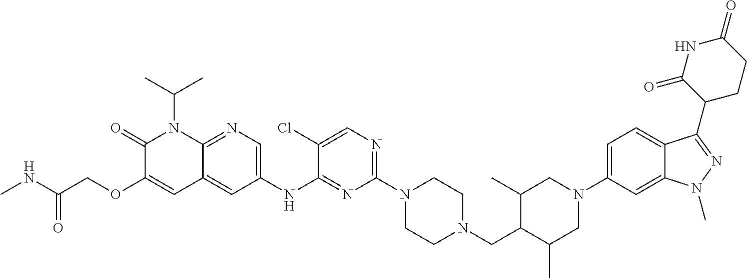 [SMILES: CNC(=O)COc1cc2cc(Nc3nc(N4CCN(CC5C(C)CN(c6ccc7c(C8CCC(=O)NC8=O)nn(C)c7c6)CC5C)CC4)ncc3Cl)cnc2n(C(C)C)c1=O] |
| 126a | 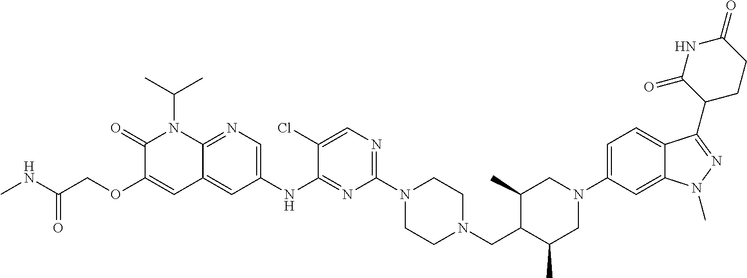 [SMILES: CNC(=O)COc1cc2cc(Nc3nc(N4CCN(CC5[C@H](C)CN(c6ccc7c(C8CCC(=O)NC8=O)nn(C)c7c6)C[C@@H]5C)CC4)ncc3Cl)cnc2n(C(C)C)c1=O] |
| 127 | 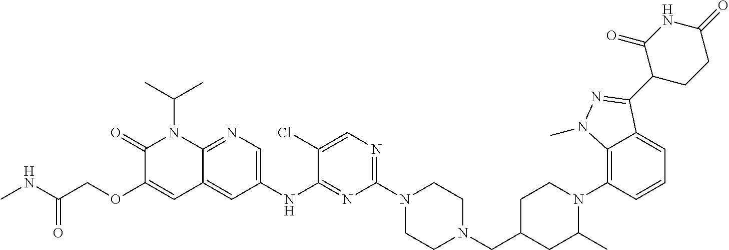 [SMILES: CNC(=O)COc1cc2cc(Nc3nc(N4CCN(CC5CCN(c6cccc7c(C8CCC(=O)NC8=O)nn(C)c67)C(C)C5)CC4)ncc3Cl)cnc2n(C(C)C)c1=O] |
| 127a | 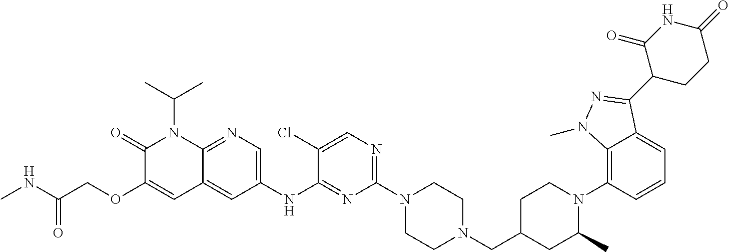 [SMILES: CNC(=O)COc1cc2cc(Nc3nc(N4CCN(CC5CCN(c6cccc7c(C8CCC(=O)NC8=O)nn(C)c67)[C@@H](C)C5)CC4)ncc3Cl)cnc2n(C(C)C)c1=O] |
| 128 | 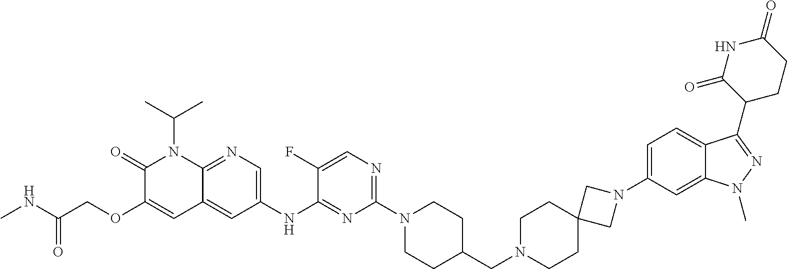 [SMILES: CNC(=O)COc1cc2cc(Nc3nc(N4CCC(CN5CCC6(CC5)CN(c5ccc7c(C8CCC(=O)NC8=O)nn(C)c7c5)C6)CC4)ncc3F)cnc2n(C(C)C)c1=O] |
| 129 | 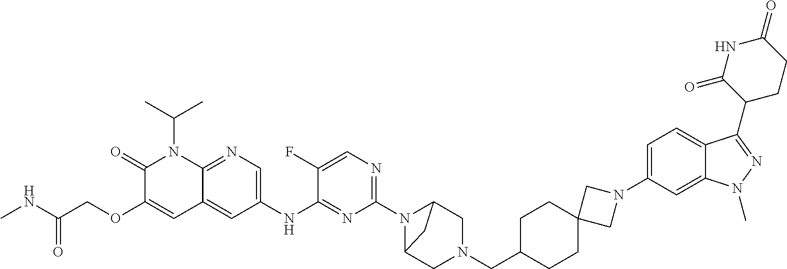 [SMILES: CNC(=O)COc1cc2cc(Nc3nc(N4C5CC4CN(CC4CCC6(CC4)CN(c4ccc7c(C8CCC(=O)NC8=O)nn(C)c7c4)C6)C5)ncc3F)cnc2n(C(C)C)c1=O] |
| 130 | 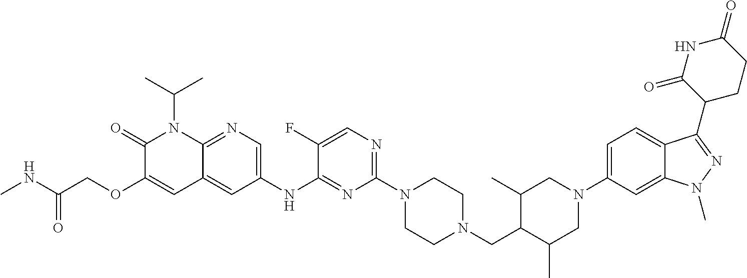 [SMILES: CNC(=O)COc1cc2cc(Nc3nc(N4CCN(CC5C(C)CN(c6ccc7c(C8CCC(=O)NC8=O)nn(C)c7c6)CC5C)CC4)ncc3F)cnc2n(C(C)C)c1=O] |
| 130a | 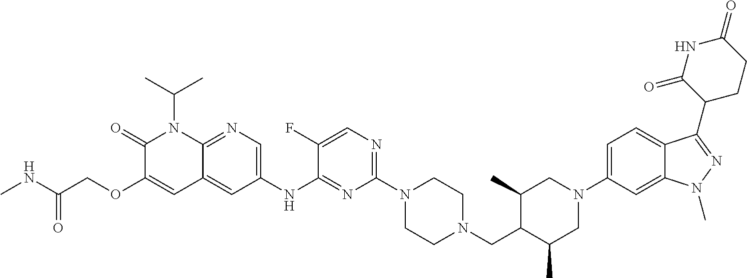 [SMILES: CNC(=O)COc1cc2cc(Nc3nc(N4CCN(CC5[C@H](C)CN(c6ccc7c(C8CCC(=O)NC8=O)nn(C)c7c6)C[C@@H]5C)CC4)ncc3F)cnc2n(C(C)C)c1=O] |
| 131 | 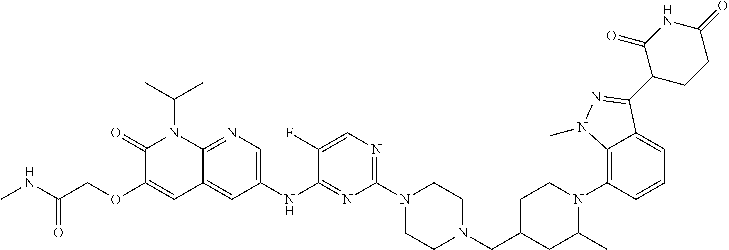 [SMILES: CNC(=O)COc1cc2cc(Nc3nc(N4CCN(CC5CCN(c6cccc7c(C8CCC(=O)NC8=O)nn(C)c67)C(C)C5)CC4)ncc3F)cnc2n(C(C)C)c1=O] |
| 131a | 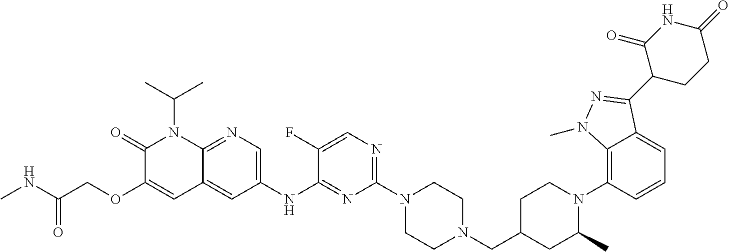 [SMILES: CNC(=O)COc1cc2cc(Nc3nc(N4CCN(CC5CCN(c6cccc7c(C8CCC(=O)NC8=O)nn(C)c67)[C@@H](C)C5)CC4)ncc3F)cnc2n(C(C)C)c1=O] |
| 132 | 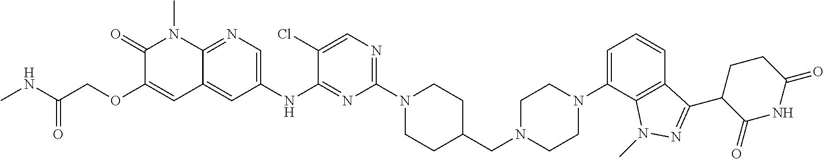 [SMILES: CNC(=O)COc1cc2cc(Nc3nc(N4CCC(CN5CCN(c6cccc7c(C8CCC(=O)NC8=O)nn(C)c67)CC5)CC4)ncc3Cl)cnc2n(C)c1=O] |
| 133 | 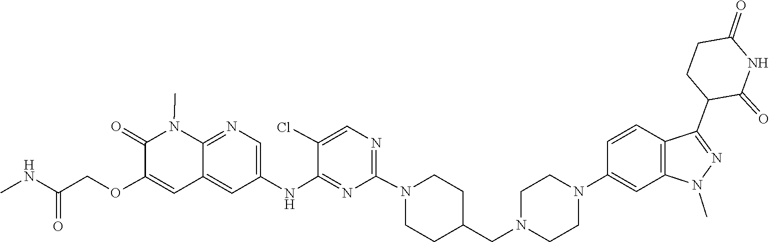 [SMILES: CNC(=O)COc1cc2cc(Nc3nc(N4CCC(CN5CCN(c6ccc7c(C8CCC(=O)NC8=O)nn(C)c7c6)CC5)CC4)ncc3Cl)cnc2n(C)c1=O] |
| 134 | 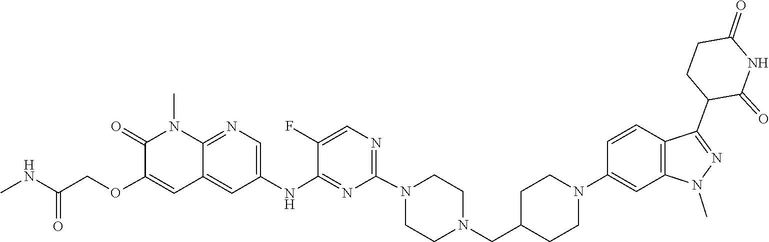 [SMILES: CNC(=O)COc1cc2cc(Nc3nc(N4CCN(CC5CCN(c6ccc7c(C8CCC(=O)NC8=O)nn(C)c7c6)CC5)CC4)ncc3F)cnc2n(C)c1=O] |
| 135 | 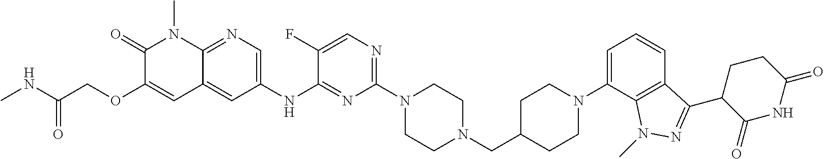 [SMILES: CNC(=O)COc1cc2cc(Nc3nc(N4CCN(CC5CCN(c6cccc7c(C8CCC(=O)NC8=O)nn(C)c67)CC5)CC4)ncc3F)cnc2n(C)c1=O] |
| 136 | 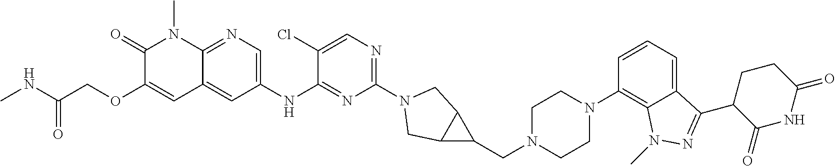 [SMILES: CNC(=O)COc1cc2cc(Nc3nc(N4CC5C(CN6CCN(c7cccc8c(C9CCC(=O)NC9=O)nn(C)c78)CC6)C5C4)ncc3Cl)cnc2n(C)c1=O] |
| 136a | 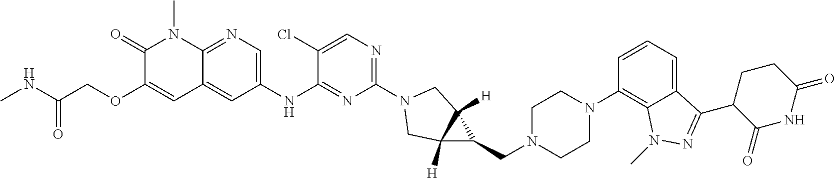 [SMILES: CNC(=O)COc1cc2cc(Nc3nc(N4C[C@@H]5[C@@H](CN6CCN(c7cccc8c(C9CCC(=O)NC9=O)nn(C)c78)CC6)[C@@H]5C4)ncc3Cl)cnc2n(C)c1=O] |
| 137 | 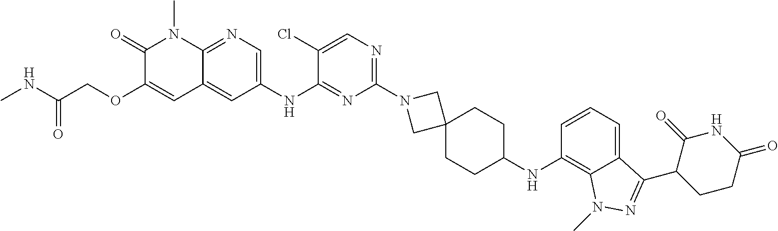 [SMILES: CNC(=O)COc1cc2cc(Nc3nc(N4CC5(CCC(Nc6cccc7c(C8CCC(=O)NC8=O)nn(C)c67)CC5)C4)ncc3Cl)cnc2n(C)c1=O] |
| 138 | 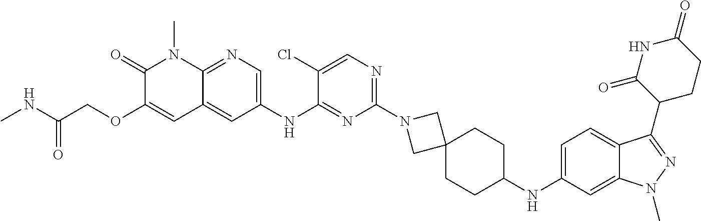 [SMILES: CNC(=O)COc1cc2cc(Nc3nc(N4CC5(CCC(Nc6ccc7c(C8CCC(=O)NC8=O)nn(C)c7c6)CC5)C4)ncc3Cl)cnc2n(C)c1=O] |
| 139 | 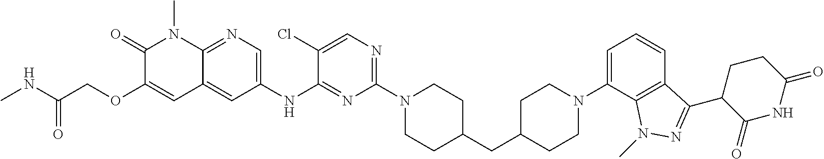 [SMILES: CNC(=O)COc1cc2cc(Nc3nc(N4CCC(CC5CCN(c6cccc7c(C8CCC(=O)NC8=O)nn(C)c67)CC5)CC4)ncc3Cl)cnc2n(C)c1=O] |
| 140 | 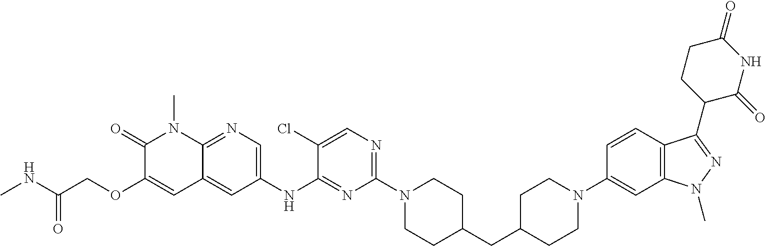 [SMILES: CNC(=O)COc1cc2cc(Nc3nc(N4CCC(CC5CCN(c6ccc7c(C8CCC(=O)NC8=O)nn(C)c7c6)CC5)CC4)ncc3Cl)cnc2n(C)c1=O] |
| 141 | 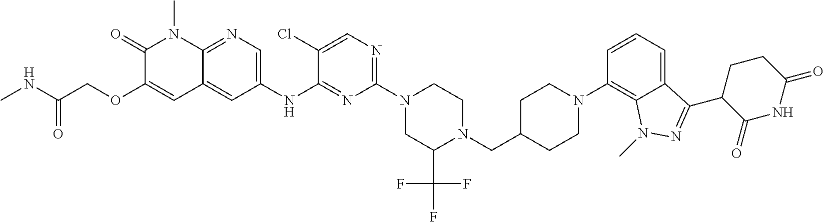 [SMILES: CNC(=O)COc1cc2cc(Nc3nc(N4CCN(CC5CCN(c6cccc7c(C8CCC(=O)NC8=O)nn(C)c67)CC5)C(C(F)(F)F)C4)ncc3Cl)cnc2n(C)c1=O] |
| 141a | 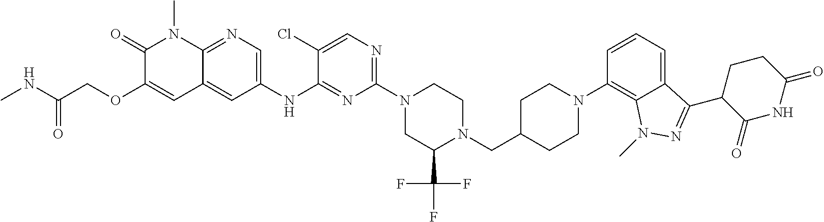 [SMILES: CNC(=O)COc1cc2cc(Nc3nc(N4CCN(CC5CCN(c6cccc7c(C8CCC(=O)NC8=O)nn(C)c67)CC5)[C@@H](C(F)(F)F)C4)ncc3Cl)cnc2n(C)c1=O] |
| 142 | 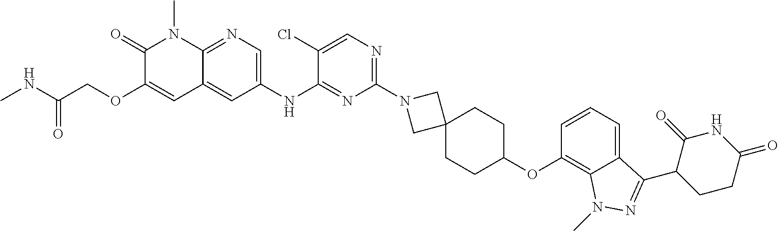 [SMILES: CNC(=O)COc1cc2cc(Nc3nc(N4CC5(CCC(Oc6cccc7c(C8CCC(=O)NC8=O)nn(C)c67)CC5)C4)ncc3Cl)cnc2n(C)c1=O] |
| 143 | 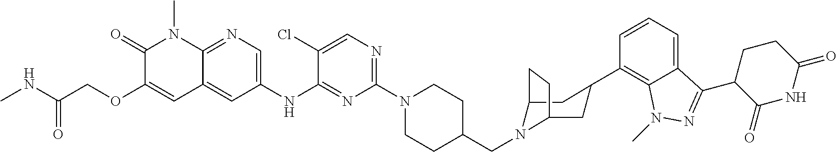 [SMILES: CNC(=O)COc1cc2cc(Nc3nc(N4CCC(CN5C6CCC5CC(c5cccc7c(C8CCC(=O)NC8=O)nn(C)c57)C6)CC4)ncc3Cl)cnc2n(C)c1=O] |
| 144 | 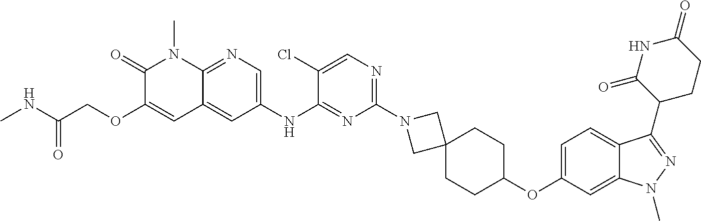 [SMILES: CNC(=O)COc1cc2cc(Nc3nc(N4CC5(CCC(Oc6ccc7c(C8CCC(=O)NC8=O)nn(C)c7c6)CC5)C4)ncc3Cl)cnc2n(C)c1=O] |
| 145 | 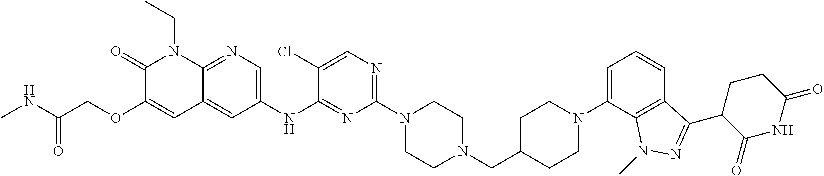 [SMILES: CCn1c(=O)c(OCC(=O)NC)cc2cc(Nc3nc(N4CCN(CC5CCN(c6cccc7c(C8CCC(=O)NC8=O)nn(C)c67)CC5)CC4)ncc3Cl)cnc21] |
| 146 | 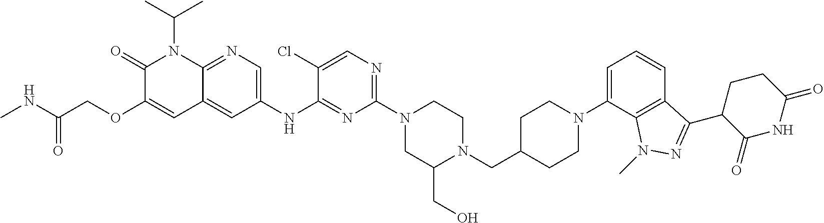 [SMILES: CNC(=O)COc1cc2cc(Nc3nc(N4CCN(CC5CCN(c6cccc7c(C8CCC(=O)NC8=O)nn(C)c67)CC5)C(CO)C4)ncc3Cl)cnc2n(C(C)C)c1=O] |
| 146a | 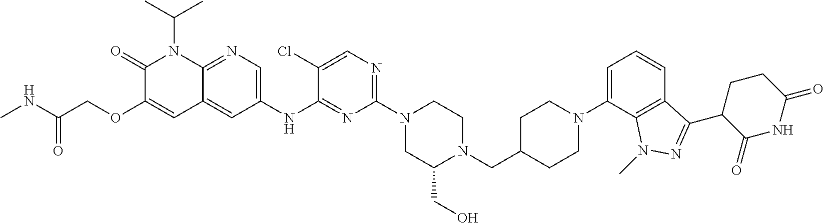 [SMILES: CNC(=O)COc1cc2cc(Nc3nc(N4CCN(CC5CCN(c6cccc7c(C8CCC(=O)NC8=O)nn(C)c67)CC5)[C@H](CO)C4)ncc3Cl)cnc2n(C(C)C)c1=O] |
| 146b | 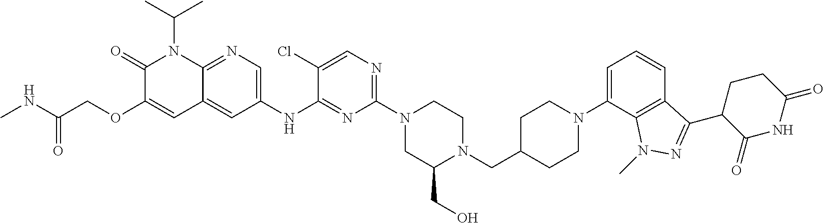 [SMILES: CNC(=O)COc1cc2cc(Nc3nc(N4CCN(CC5CCN(c6cccc7c(C8CCC(=O)NC8=O)nn(C)c67)CC5)[C@@H](CO)C4)ncc3Cl)cnc2n(C(C)C)c1=O] |
| 147 | 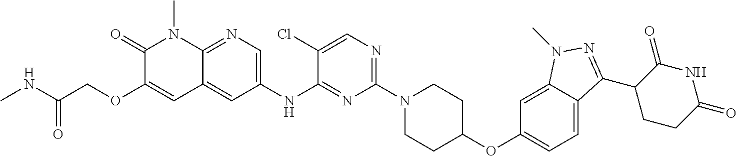 [SMILES: CNC(=O)COc1cc2cc(Nc3nc(N4CCC(Oc5ccc6c(C7CCC(=O)NC7=O)nn(C)c6c5)CC4)ncc3Cl)cnc2n(C)c1=O] |
| 148 | 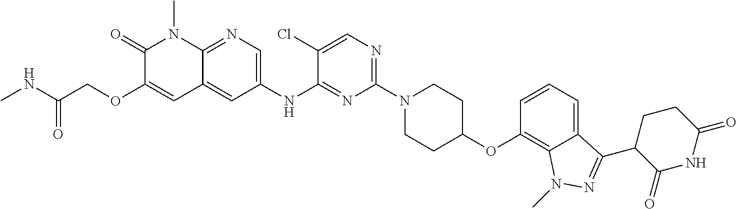 [SMILES: CNC(=O)COc1cc2cc(Nc3nc(N4CCC(Oc5cccc6c(C7CCC(=O)NC7=O)nn(C)c56)CC4)ncc3Cl)cnc2n(C)c1=O] |
| 149 | 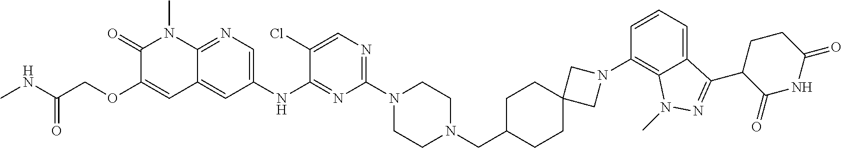 [SMILES: CNC(=O)COc1cc2cc(Nc3nc(N4CCN(CC5CCC6(CC5)CN(c5cccc7c(C8CCC(=O)NC8=O)nn(C)c57)C6)CC4)ncc3Cl)cnc2n(C)c1=O] |
| 150 | 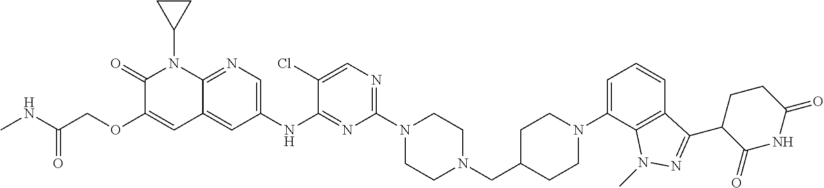 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)cnc2n(C2CC2)c1=O] |
| 151 | 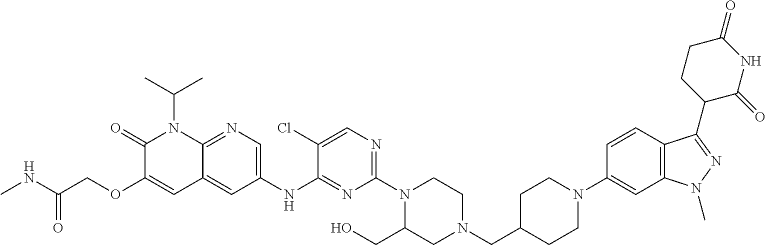 [SMILES: CNC(=O)COc1cc2cc(Nc3nc(N4CCN(CC5CCN(c6ccc7c(C8CCC(=O)NC8=O)nn(C)c7c6)CC5)CC4CO)ncc3Cl)cnc2n(C(C)C)c1=O] |
| 151a | 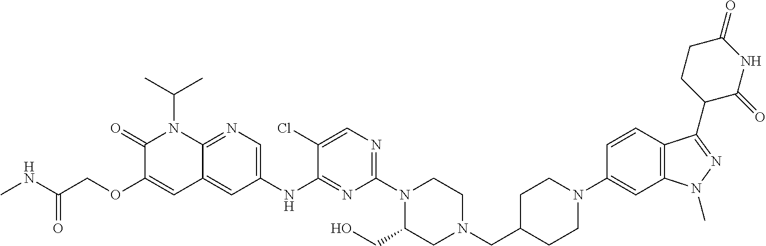 [SMILES: CNC(=O)COc1cc2cc(Nc3nc(N4CCN(CC5CCN(c6ccc7c(C8CCC(=O)NC8=O)nn(C)c7c6)CC5)C[C@@H]4CO)ncc3Cl)cnc2n(C(C)C)c1=O] |
| 151b | 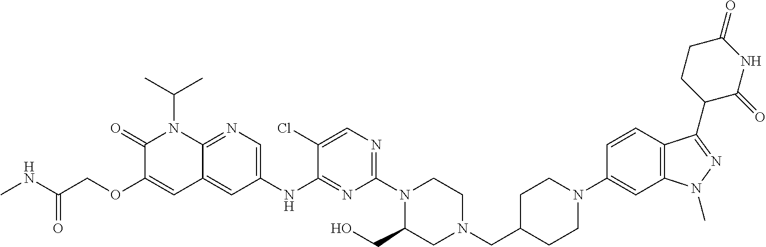 [SMILES: CNC(=O)COc1cc2cc(Nc3nc(N4CCN(CC5CCN(c6ccc7c(C8CCC(=O)NC8=O)nn(C)c7c6)CC5)C[C@H]4CO)ncc3Cl)cnc2n(C(C)C)c1=O] |
| 152 | 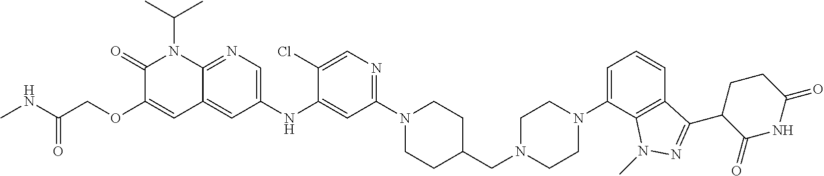 [SMILES: CNC(=O)COc1cc2cc(Nc3cc(N4CCC(CN5CCN(c6cccc7c(C8CCC(=O)NC8=O)nn(C)c67)CC5)CC4)ncc3Cl)cnc2n(C(C)C)c1=O] |
| 153 | 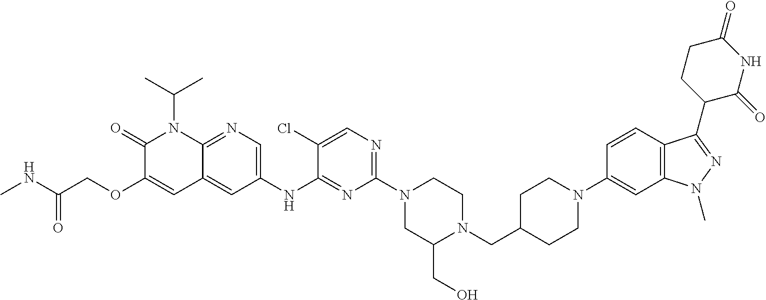 [SMILES: CNC(=O)COc1cc2cc(Nc3nc(N4CCN(CC5CCN(c6ccc7c(C8CCC(=O)NC8=O)nn(C)c7c6)CC5)C(CO)C4)ncc3Cl)cnc2n(C(C)C)c1=O] |
| 153a | 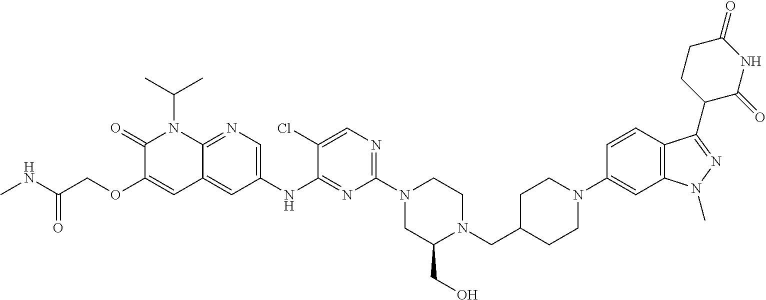 [SMILES: CNC(=O)COc1cc2cc(Nc3nc(N4CCN(CC5CCN(c6ccc7c(C8CCC(=O)NC8=O)nn(C)c7c6)CC5)[C@@H](CO)C4)ncc3Cl)cnc2n(C(C)C)c1=O] |
| 153b | 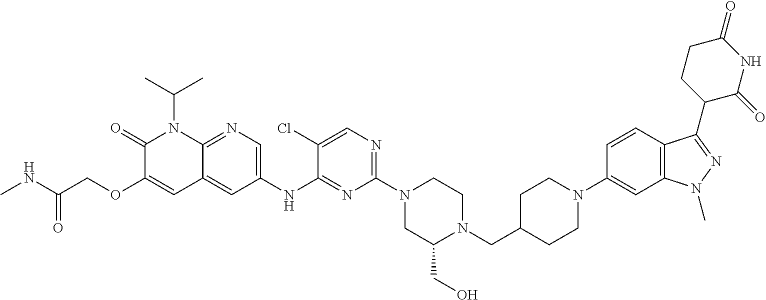 [SMILES: CNC(=O)COc1cc2cc(Nc3nc(N4CCN(CC5CCN(c6ccc7c(C8CCC(=O)NC8=O)nn(C)c7c6)CC5)[C@H](CO)C4)ncc3Cl)cnc2n(C(C)C)c1=O] |
| 154 | 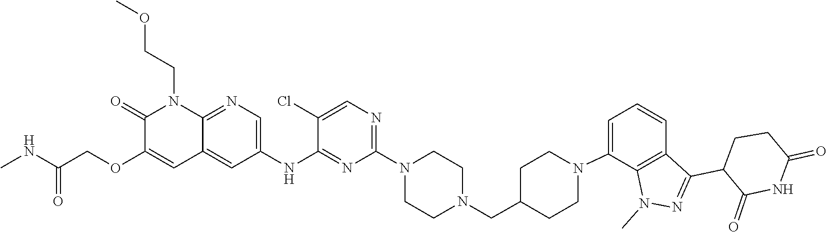 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)cnc2n(CCOC)c1=O] |
| 155 | 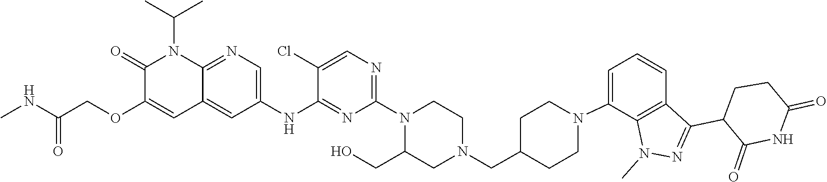 [SMILES: CNC(=O)COc1cc2cc(Nc3nc(N4CCN(CC5CCN(c6cccc7c(C8CCC(=O)NC8=O)nn(C)c67)CC5)CC4CO)ncc3Cl)cnc2n(C(C)C)c1=O] |
| 155a | 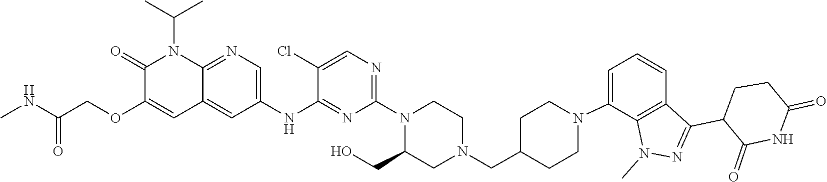 [SMILES: CNC(=O)COc1cc2cc(Nc3nc(N4CCN(CC5CCN(c6cccc7c(C8CCC(=O)NC8=O)nn(C)c67)CC5)C[C@H]4CO)ncc3Cl)cnc2n(C(C)C)c1=O] |
| 155b | 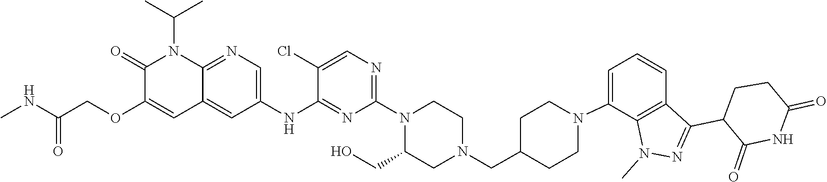 [SMILES: CNC(=O)COc1cc2cc(Nc3nc(N4CCN(CC5CCN(c6cccc7c(C8CCC(=O)NC8=O)nn(C)c67)CC5)C[C@@H]4CO)ncc3Cl)cnc2n(C(C)C)c1=O] |
| 156 | 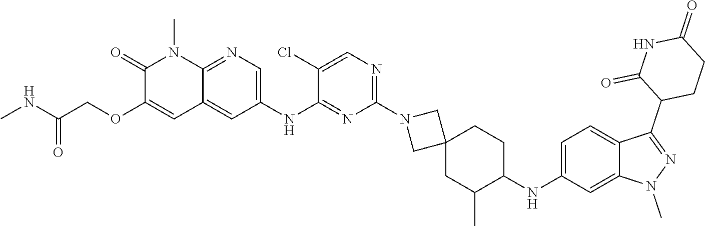 [SMILES: CNC(=O)COc1cc2cc(Nc3nc(N4CC5(CCC(Nc6ccc7c(C8CCC(=O)NC8=O)nn(C)c7c6)C(C)C5)C4)ncc3Cl)cnc2n(C)c1=O] |
| 156a | 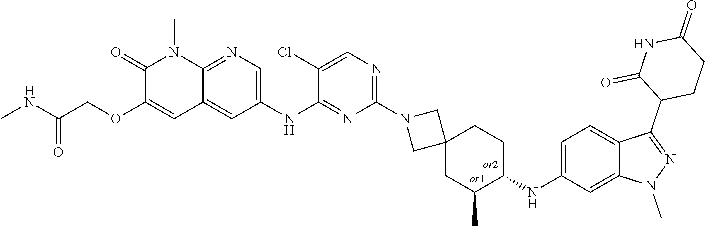 [SMILES: CNC(=O)COc1cc2cc(Nc3nc(N4CC5(CC[C@H](Nc6ccc7c(C8CCC(=O)NC8=O)nn(C)c7c6)[C@@H](C)C5)C4)ncc3Cl)cnc2n(C)c1=O] |
| 156b | 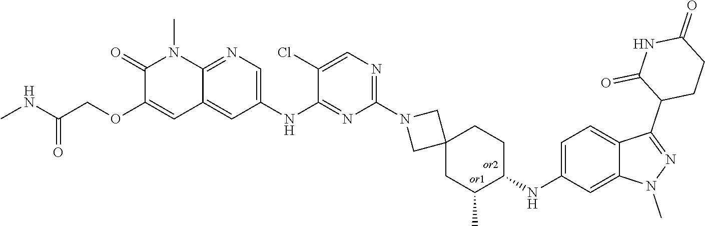 [SMILES: CNC(=O)COc1cc2cc(Nc3nc(N4CC5(CC[C@H](Nc6ccc7c(C8CCC(=O)NC8=O)nn(C)c7c6)[C@H](C)C5)C4)ncc3Cl)cnc2n(C)c1=O] |
| 156c | 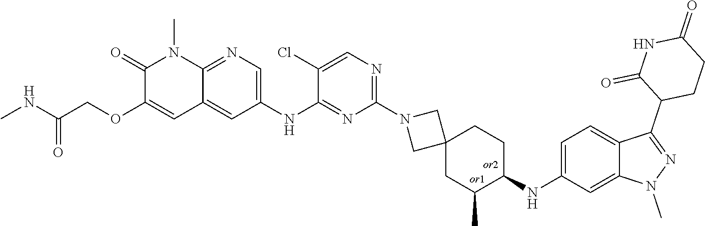 [SMILES: CNC(=O)COc1cc2cc(Nc3nc(N4CC5(CC[C@@H](Nc6ccc7c(C8CCC(=O)NC8=O)nn(C)c7c6)[C@@H](C)C5)C4)ncc3Cl)cnc2n(C)c1=O] |
| 156d | 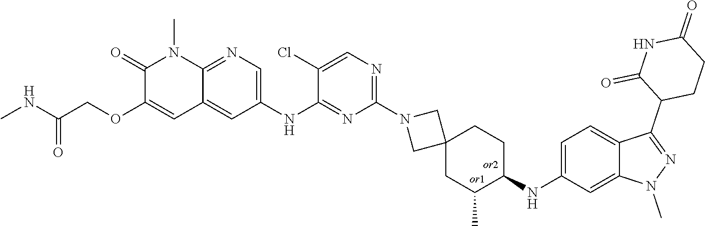 [SMILES: CNC(=O)COc1cc2cc(Nc3nc(N4CC5(CC[C@@H](Nc6ccc7c(C8CCC(=O)NC8=O)nn(C)c7c6)[C@H](C)C5)C4)ncc3Cl)cnc2n(C)c1=O] |
| 157 | 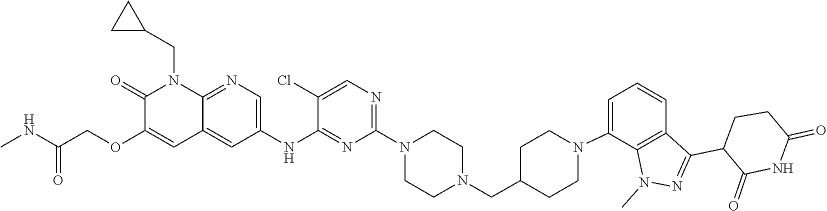 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)cnc2n(CC2CC2)c1=O] |
| 158 | 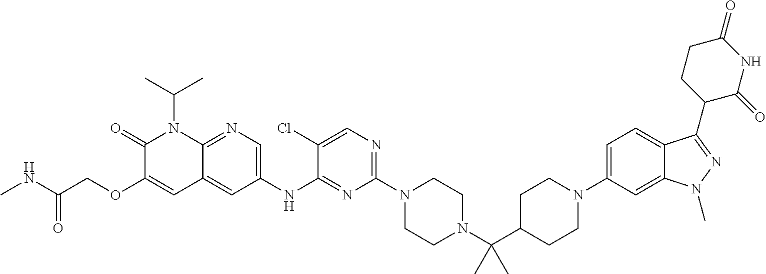 [SMILES: CNC(=O)COc1cc2cc(Nc3nc(N4CCN(C(C)(C)C5CCN(c6ccc7c(C8CCC(=O)NC8=O)nn(C)c7c6)CC5)CC4)ncc3Cl)cnc2n(C(C)C)c1=O] |
| 159 | 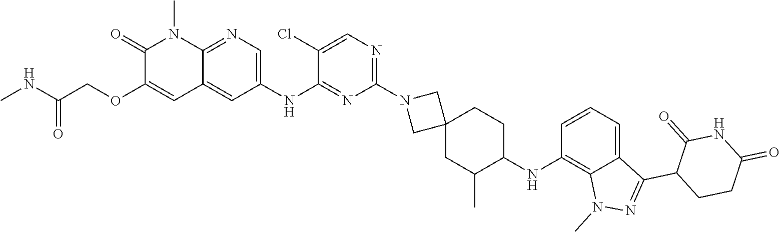 [SMILES: CNC(=O)COc1cc2cc(Nc3nc(N4CC5(CCC(Nc6cccc7c(C8CCC(=O)NC8=O)nn(C)c67)C(C)C5)C4)ncc3Cl)cnc2n(C)c1=O] |
| 159a | 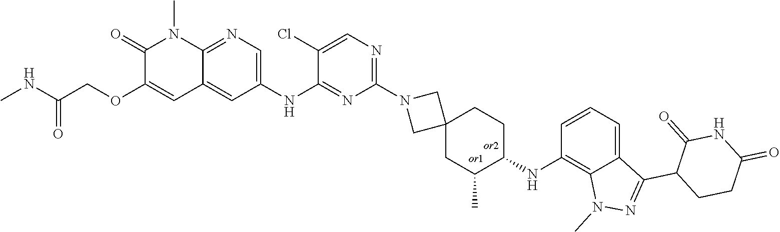 [SMILES: CNC(=O)COc1cc2cc(Nc3nc(N4CC5(CC[C@H](Nc6cccc7c(C8CCC(=O)NC8=O)nn(C)c67)[C@H](C)C5)C4)ncc3Cl)cnc2n(C)c1=O] |
| 159b | 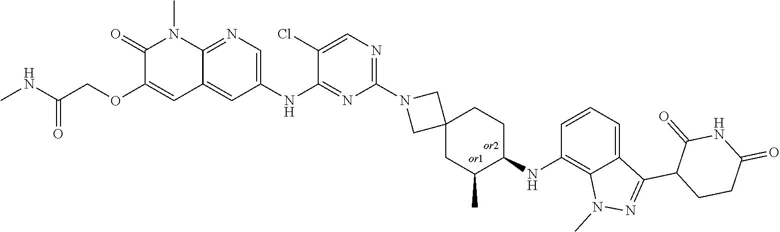 [SMILES: CNC(=O)COc1cc2cc(Nc3nc(N4CC5(CC[C@@H](Nc6cccc7c(C8CCC(=O)NC8=O)nn(C)c67)[C@@H](C)C5)C4)ncc3Cl)cnc2n(C)c1=O] |
| 159c | 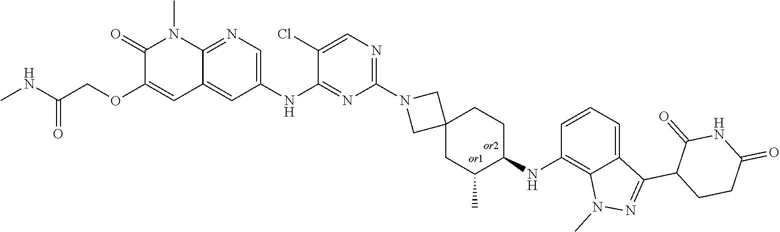 [SMILES: CNC(=O)COc1cc2cc(Nc3nc(N4CC5(CC[C@@H](Nc6cccc7c(C8CCC(=O)NC8=O)nn(C)c67)[C@H](C)C5)C4)ncc3Cl)cnc2n(C)c1=O] |
| 159d | 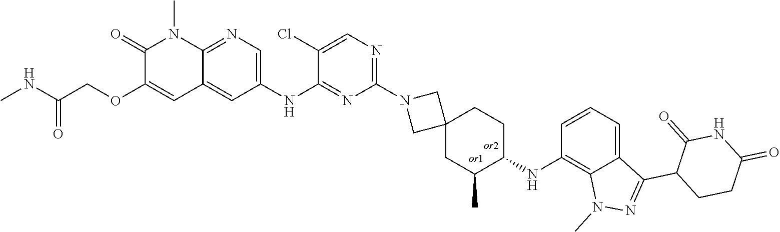 [SMILES: CNC(=O)COc1cc2cc(Nc3nc(N4CC5(CC[C@H](Nc6cccc7c(C8CCC(=O)NC8=O)nn(C)c67)[C@@H](C)C5)C4)ncc3Cl)cnc2n(C)c1=O] |
| 160 | 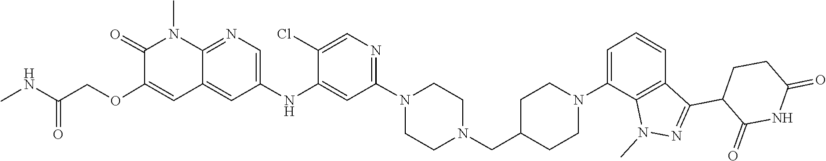 [SMILES: CNC(=O)COc1cc2cc(Nc3cc(N4CCN(CC5CCN(c6cccc7c(C8CCC(=O)NC8=O)nn(C)c67)CC5)CC4)ncc3Cl)cnc2n(C)c1=O] |
| 161 | 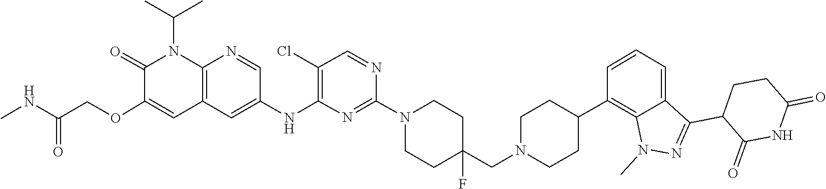 [SMILES: CNC(=O)COc1cc2cc(Nc3nc(N4CCC(F)(CN5CCC(c6cccc7c(C8CCC(=O)NC8=O)nn(C)c67)CC5)CC4)ncc3Cl)cnc2n(C(C)C)c1=O] |
| 162 | 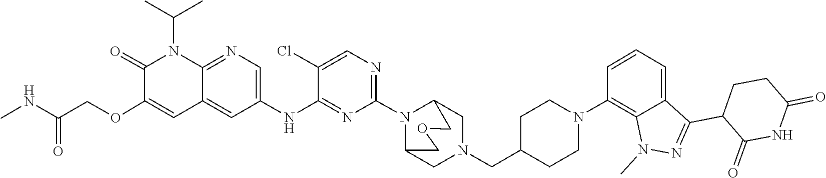 [SMILES: CNC(=O)COc1cc2cc(Nc3nc(N4C5COCC4CN(CC4CCN(c6cccc7c(C8CCC(=O)NC8=O)nn(C)c67)CC4)C5)ncc3Cl)cnc2n(C(C)C)c1=O] |
| 163 | 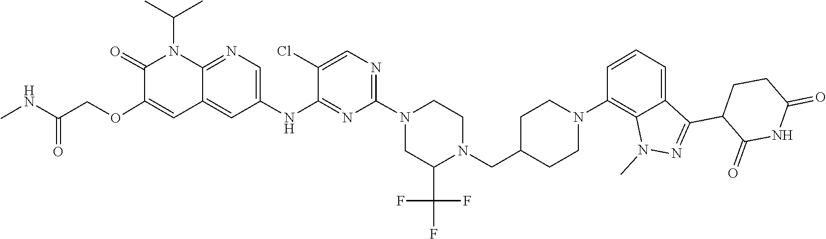 [SMILES: CNC(=O)COc1cc2cc(Nc3nc(N4CCN(CC5CCN(c6cccc7c(C8CCC(=O)NC8=O)nn(C)c67)CC5)C(C(F)(F)F)C4)ncc3Cl)cnc2n(C(C)C)c1=O] |
| 163a | 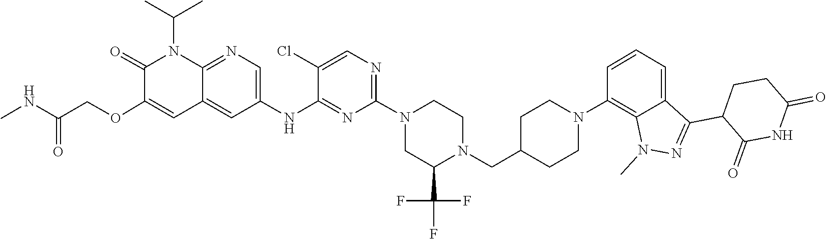 [SMILES: CNC(=O)COc1cc2cc(Nc3nc(N4CCN(CC5CCN(c6cccc7c(C8CCC(=O)NC8=O)nn(C)c67)CC5)[C@@H](C(F)(F)F)C4)ncc3Cl)cnc2n(C(C)C)c1=O] |
| 164 | 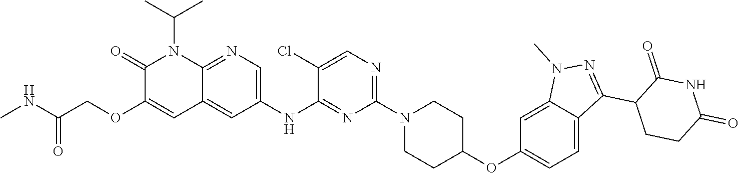 [SMILES: CNC(=O)COc1cc2cc(Nc3nc(N4CCC(Oc5ccc6c(C7CCC(=O)NC7=O)nn(C)c6c5)CC4)ncc3Cl)cnc2n(C(C)C)c1=O] |
| 165 | 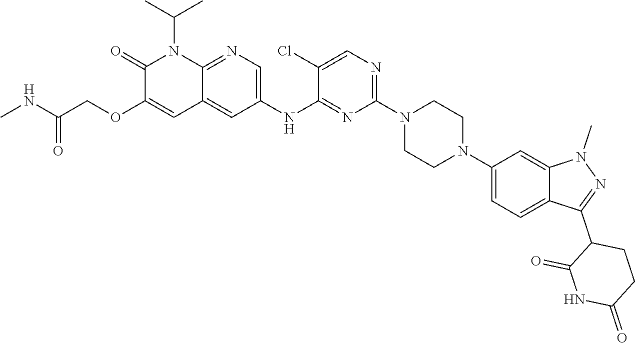 [SMILES: CNC(=O)COc1cc2cc(Nc3nc(N4CCN(c5ccc6c(C7CCC(=O)NC7=O)nn(C)c6c5)CC4)ncc3Cl)cnc2n(C(C)C)c1=O] |
| 166 | 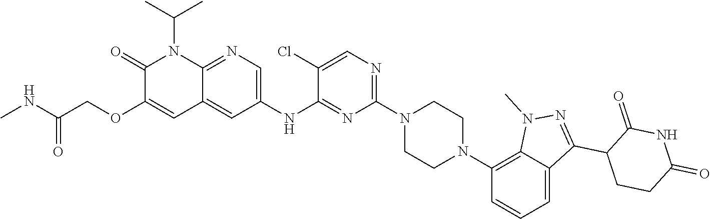 [SMILES: CNC(=O)COc1cc2cc(Nc3nc(N4CCN(c5cccc6c(C7CCC(=O)NC7=O)nn(C)c56)CC4)ncc3Cl)cnc2n(C(C)C)c1=O] |
| 167 | 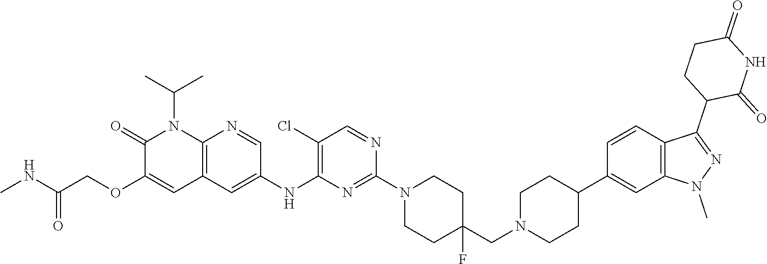 [SMILES: CNC(=O)COc1cc2cc(Nc3nc(N4CCC(F)(CN5CCC(c6ccc7c(C8CCC(=O)NC8=O)nn(C)c7c6)CC5)CC4)ncc3Cl)cnc2n(C(C)C)c1=O] |
| 168 | 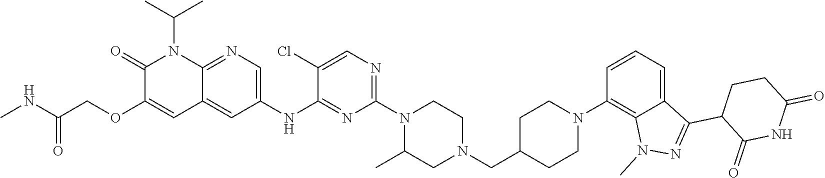 [SMILES: CNC(=O)COc1cc2cc(Nc3nc(N4CCN(CC5CCN(c6cccc7c(C8CCC(=O)NC8=O)nn(C)c67)CC5)CC4C)ncc3Cl)cnc2n(C(C)C)c1=O] |
| 168a | 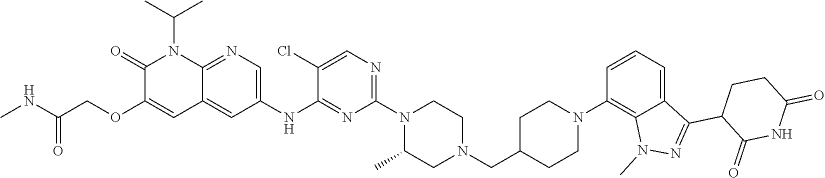 [SMILES: CNC(=O)COc1cc2cc(Nc3nc(N4CCN(CC5CCN(c6cccc7c(C8CCC(=O)NC8=O)nn(C)c67)CC5)C[C@@H]4C)ncc3Cl)cnc2n(C(C)C)c1=O] |
| 168b | 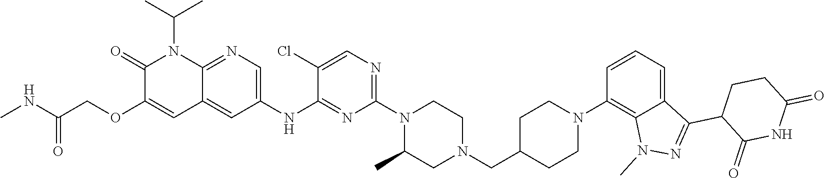 [SMILES: CNC(=O)COc1cc2cc(Nc3nc(N4CCN(CC5CCN(c6cccc7c(C8CCC(=O)NC8=O)nn(C)c67)CC5)C[C@H]4C)ncc3Cl)cnc2n(C(C)C)c1=O] |
| 169 | 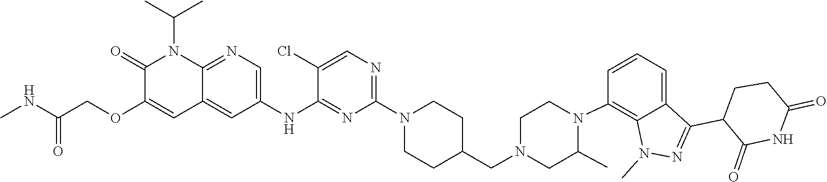 [SMILES: CNC(=O)COc1cc2cc(Nc3nc(N4CCC(CN5CCN(c6cccc7c(C8CCC(=O)NC8=O)nn(C)c67)C(C)C5)CC4)ncc3Cl)cnc2n(C(C)C)c1=O] |
| 169a | 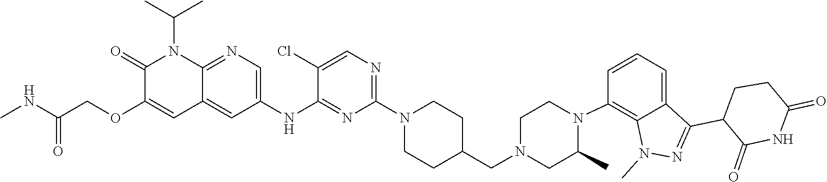 [SMILES: CNC(=O)COc1cc2cc(Nc3nc(N4CCC(CN5CCN(c6cccc7c(C8CCC(=O)NC8=O)nn(C)c67)[C@@H](C)C5)CC4)ncc3Cl)cnc2n(C(C)C)c1=O] |
| 169b | 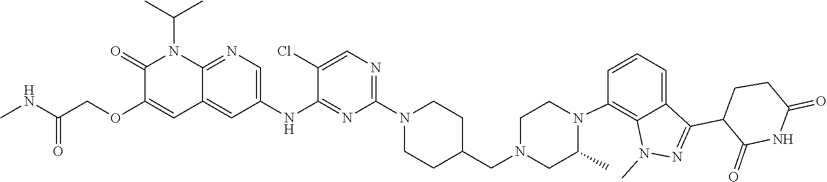 [SMILES: CNC(=O)COc1cc2cc(Nc3nc(N4CCC(CN5CCN(c6cccc7c(C8CCC(=O)NC8=O)nn(C)c67)[C@H](C)C5)CC4)ncc3Cl)cnc2n(C(C)C)c1=O] |
| 170 | 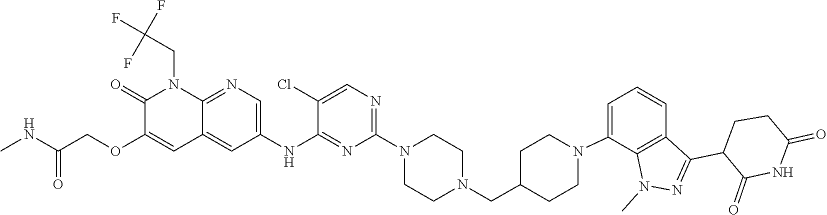 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)cnc2n(CC(F)(F)F)c1=O] |
| 171 | 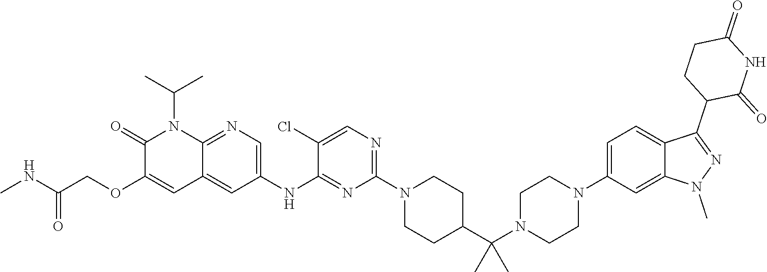 [SMILES: CNC(=O)COc1cc2cc(Nc3nc(N4CCC(C(C)(C)N5CCN(c6ccc7c(C8CCC(=O)NC8=O)nn(C)c7c6)CC5)CC4)ncc3Cl)cnc2n(C(C)C)c1=O] |
| 172 | 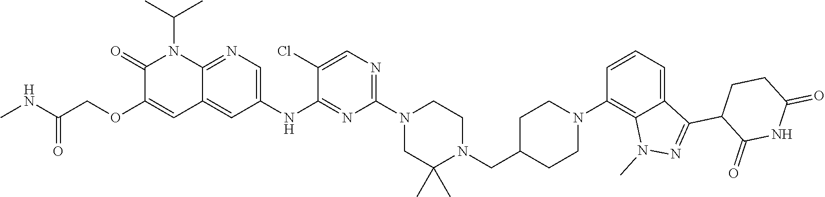 [SMILES: CNC(=O)COc1cc2cc(Nc3nc(N4CCN(CC5CCN(c6cccc7c(C8CCC(=O)NC8=O)nn(C)c67)CC5)C(C)(C)C4)ncc3Cl)cnc2n(C(C)C)c1=O] |
| 173 | 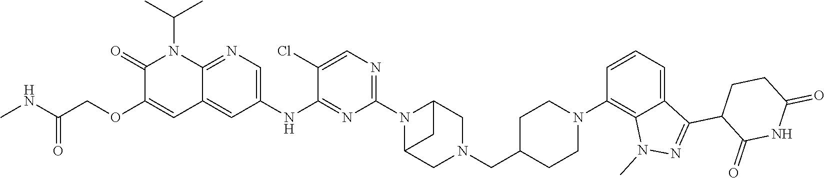 [SMILES: CNC(=O)COc1cc2cc(Nc3nc(N4C5CC4CN(CC4CCN(c6cccc7c(C8CCC(=O)NC8=O)nn(C)c67)CC4)C5)ncc3Cl)cnc2n(C(C)C)c1=O] |
| 174 | 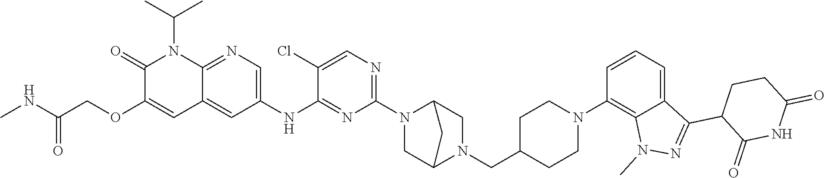 [SMILES: CNC(=O)COc1cc2cc(Nc3nc(N4CC5CC4CN5CC4CCN(c5cccc6c(C7CCC(=O)NC7=O)nn(C)c56)CC4)ncc3Cl)cnc2n(C(C)C)c1=O] |
| 174a | 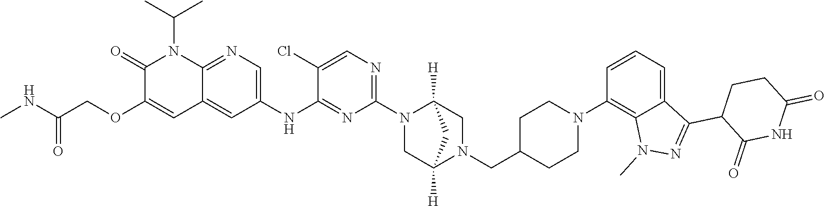 [SMILES: CNC(=O)COc1cc2cc(Nc3nc(N4C[C@H]5C[C@@H]4CN5CC4CCN(c5cccc6c(C7CCC(=O)NC7=O)nn(C)c56)CC4)ncc3Cl)cnc2n(C(C)C)c1=O] |
| 174b | 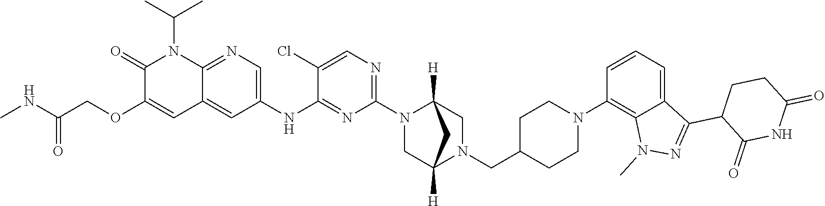 [SMILES: CNC(=O)COc1cc2cc(Nc3nc(N4C[C@@H]5C[C@H]4CN5CC4CCN(c5cccc6c(C7CCC(=O)NC7=O)nn(C)c56)CC4)ncc3Cl)cnc2n(C(C)C)c1=O] |
| 175 | 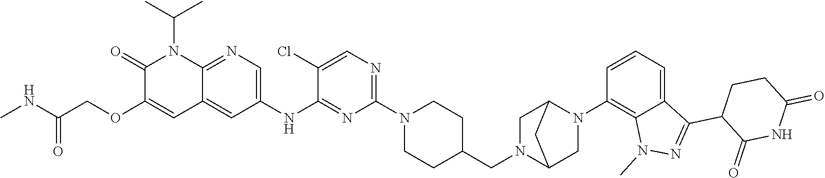 [SMILES: CNC(=O)COc1cc2cc(Nc3nc(N4CCC(CN5CC6CC5CN6c5cccc6c(C7CCC(=O)NC7=O)nn(C)c56)CC4)ncc3Cl)cnc2n(C(C)C)c1=O] |
| 175a | 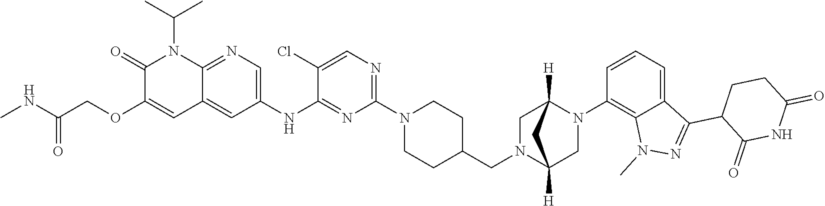 [SMILES: CNC(=O)COc1cc2cc(Nc3nc(N4CCC(CN5C[C@H]6C[C@@H]5CN6c5cccc6c(C7CCC(=O)NC7=O)nn(C)c56)CC4)ncc3Cl)cnc2n(C(C)C)c1=O] |
| 175b | 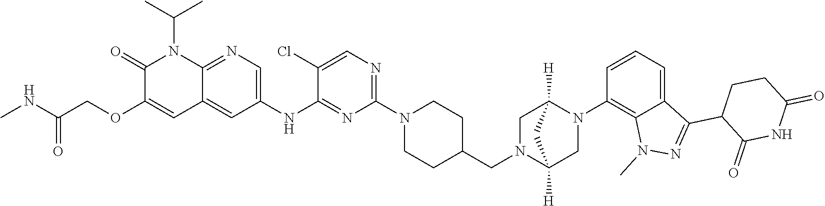 [SMILES: CNC(=O)COc1cc2cc(Nc3nc(N4CCC(CN5C[C@@H]6C[C@H]5CN6c5cccc6c(C7CCC(=O)NC7=O)nn(C)c56)CC4)ncc3Cl)cnc2n(C(C)C)c1=O] |
| 176 | 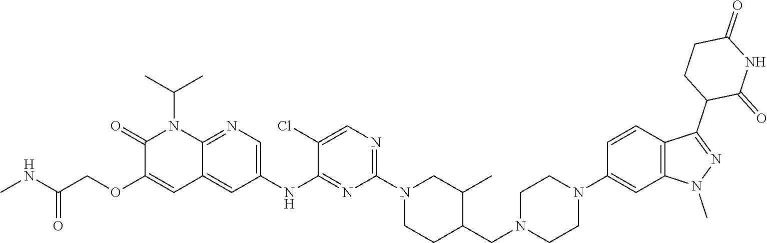 [SMILES: CNC(=O)COc1cc2cc(Nc3nc(N4CCC(CN5CCN(c6ccc7c(C8CCC(=O)NC8=O)nn(C)c7c6)CC5)C(C)C4)ncc3Cl)cnc2n(C(C)C)c1=O] |
| 176a | 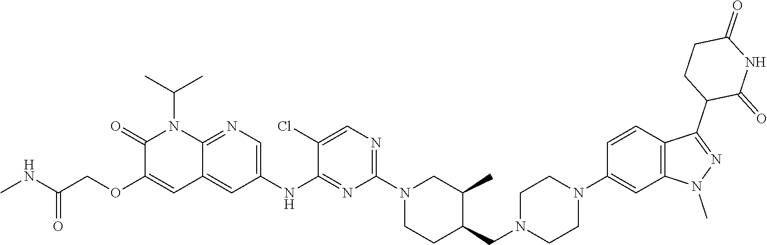 [SMILES: CNC(=O)COc1cc2cc(Nc3nc(N4CC[C@H](CN5CCN(c6ccc7c(C8CCC(=O)NC8=O)nn(C)c7c6)CC5)[C@H](C)C4)ncc3Cl)cnc2n(C(C)C)c1=O] |
| 176b | 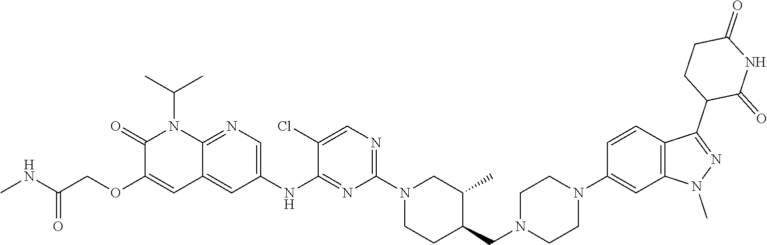 [SMILES: CNC(=O)COc1cc2cc(Nc3nc(N4CC[C@H](CN5CCN(c6ccc7c(C8CCC(=O)NC8=O)nn(C)c7c6)CC5)[C@@H](C)C4)ncc3Cl)cnc2n(C(C)C)c1=O] |
| 176c | 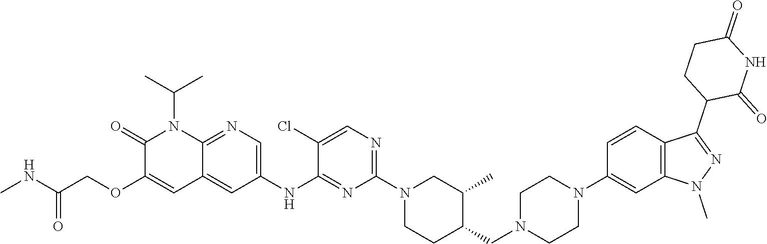 [SMILES: CNC(=O)COc1cc2cc(Nc3nc(N4CC[C@@H](CN5CCN(c6ccc7c(C8CCC(=O)NC8=O)nn(C)c7c6)CC5)[C@@H](C)C4)ncc3Cl)cnc2n(C(C)C)c1=O] |
| 176d | 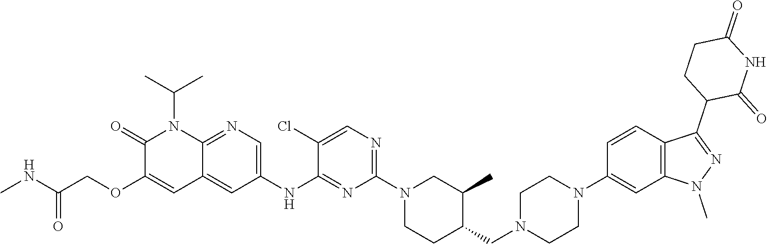 [SMILES: CNC(=O)COc1cc2cc(Nc3nc(N4CC[C@@H](CN5CCN(c6ccc7c(C8CCC(=O)NC8=O)nn(C)c7c6)CC5)[C@H](C)C4)ncc3Cl)cnc2n(C(C)C)c1=O] |
| 177 | 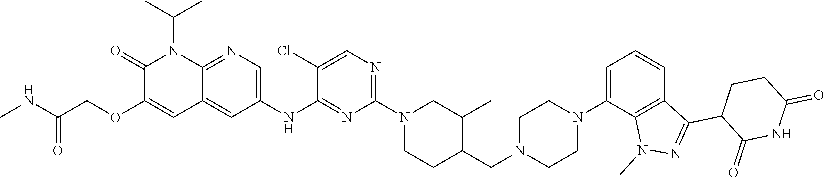 [SMILES: CNC(=O)COc1cc2cc(Nc3nc(N4CCC(CN5CCN(c6cccc7c(C8CCC(=O)NC8=O)nn(C)c67)CC5)C(C)C4)ncc3Cl)cnc2n(C(C)C)c1=O] |
| 177a | 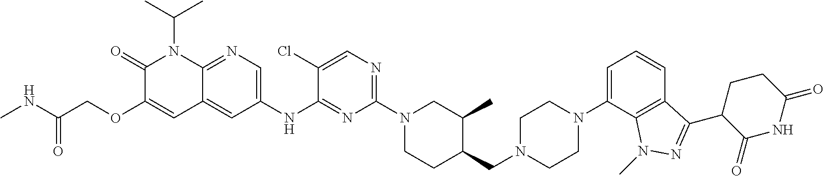 [SMILES: CNC(=O)COc1cc2cc(Nc3nc(N4CC[C@H](CN5CCN(c6cccc7c(C8CCC(=O)NC8=O)nn(C)c67)CC5)[C@H](C)C4)ncc3Cl)cnc2n(C(C)C)c1=O] |
| 177b | 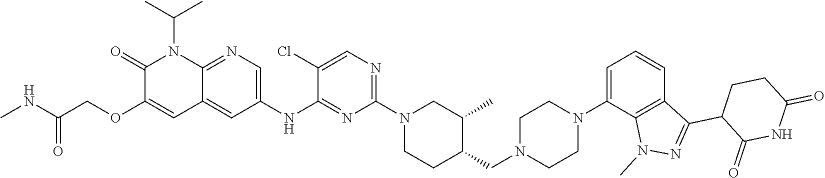 [SMILES: CNC(=O)COc1cc2cc(Nc3nc(N4CC[C@@H](CN5CCN(c6cccc7c(C8CCC(=O)NC8=O)nn(C)c67)CC5)[C@@H](C)C4)ncc3Cl)cnc2n(C(C)C)c1=O] |
| 177c | 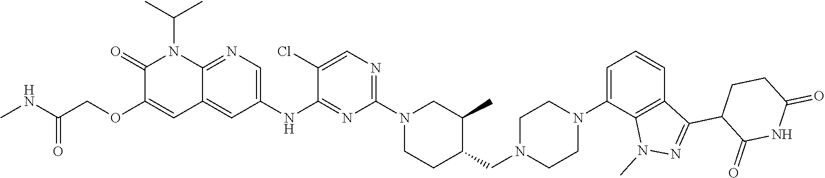 [SMILES: CNC(=O)COc1cc2cc(Nc3nc(N4CC[C@@H](CN5CCN(c6cccc7c(C8CCC(=O)NC8=O)nn(C)c67)CC5)[C@H](C)C4)ncc3Cl)cnc2n(C(C)C)c1=O] |
| 177d | 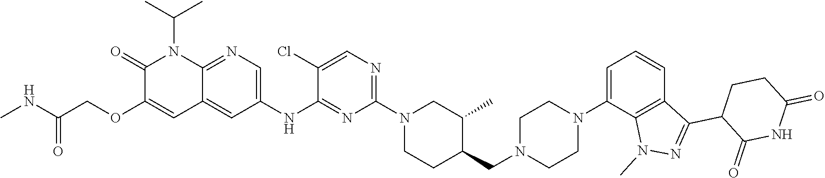 [SMILES: CNC(=O)COc1cc2cc(Nc3nc(N4CC[C@H](CN5CCN(c6cccc7c(C8CCC(=O)NC8=O)nn(C)c67)CC5)[C@@H](C)C4)ncc3Cl)cnc2n(C(C)C)c1=O] |
| 178 | 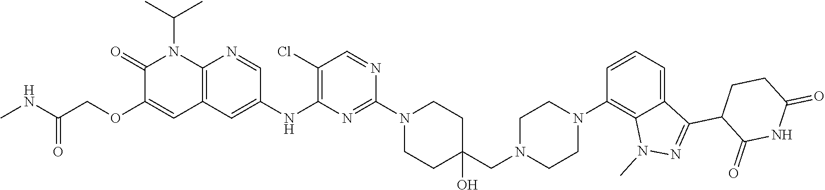 [SMILES: CNC(=O)COc1cc2cc(Nc3nc(N4CCC(O)(CN5CCN(c6cccc7c(C8CCC(=O)NC8=O)nn(C)c67)CC5)CC4)ncc3Cl)cnc2n(C(C)C)c1=O] |
| 179 | 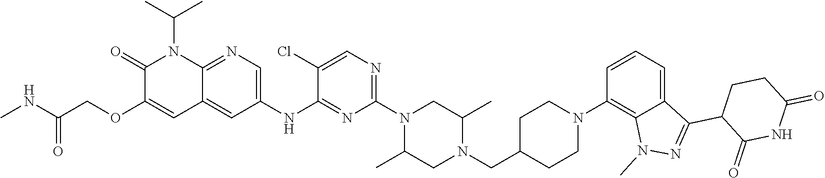 [SMILES: CNC(=O)COc1cc2cc(Nc3nc(N4CC(C)N(CC5CCN(c6cccc7c(C8CCC(=O)NC8=O)nn(C)c67)CC5)CC4C)ncc3Cl)cnc2n(C(C)C)c1=O] |
| 179a | 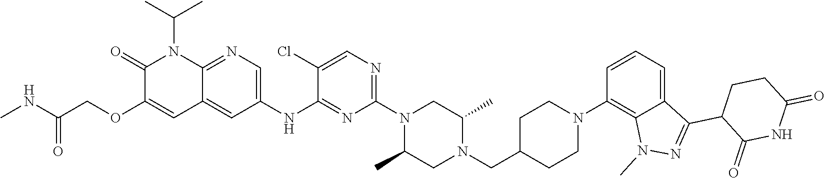 [SMILES: CNC(=O)COc1cc2cc(Nc3nc(N4C[C@H](C)N(CC5CCN(c6cccc7c(C8CCC(=O)NC8=O)nn(C)c67)CC5)C[C@H]4C)ncc3Cl)cnc2n(C(C)C)c1=O] |
| 179b | 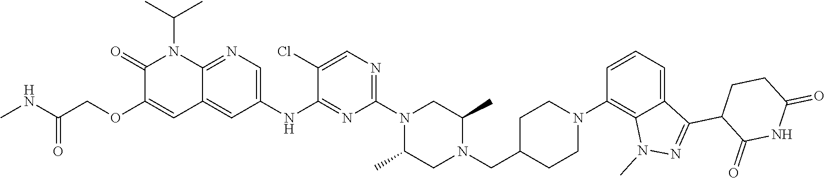 [SMILES: CNC(=O)COc1cc2cc(Nc3nc(N4C[C@@H](C)N(CC5CCN(c6cccc7c(C8CCC(=O)NC8=O)nn(C)c67)CC5)C[C@@H]4C)ncc3Cl)cnc2n(C(C)C)c1=O] |
| 180 | 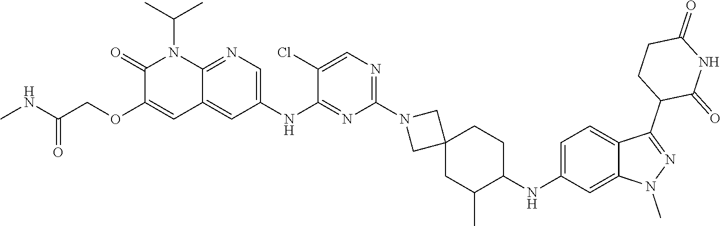 [SMILES: CNC(=O)COc1cc2cc(Nc3nc(N4CC5(CCC(Nc6ccc7c(C8CCC(=O)NC8=O)nn(C)c7c6)C(C)C5)C4)ncc3Cl)cnc2n(C(C)C)c1=O] |
| 180a | 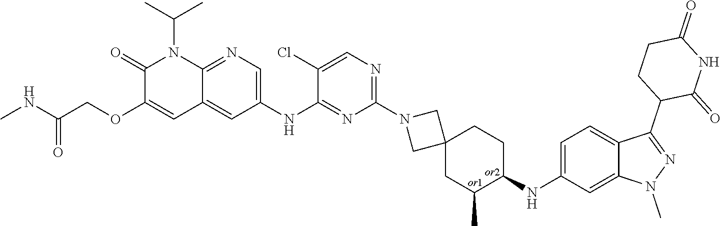 [SMILES: CNC(=O)COc1cc2cc(Nc3nc(N4CC5(CC[C@@H](Nc6ccc7c(C8CCC(=O)NC8=O)nn(C)c7c6)[C@@H](C)C5)C4)ncc3Cl)cnc2n(C(C)C)c1=O] |
| 180b | 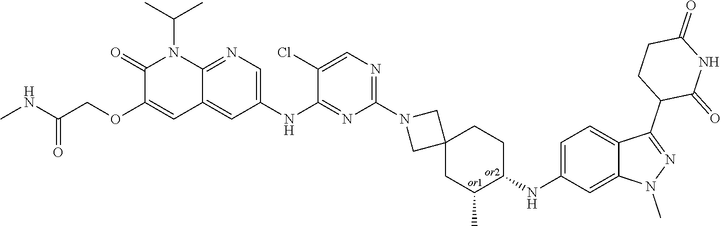 [SMILES: CNC(=O)COc1cc2cc(Nc3nc(N4CC5(CC[C@H](Nc6ccc7c(C8CCC(=O)NC8=O)nn(C)c7c6)[C@H](C)C5)C4)ncc3Cl)cnc2n(C(C)C)c1=O] |
| 180c | 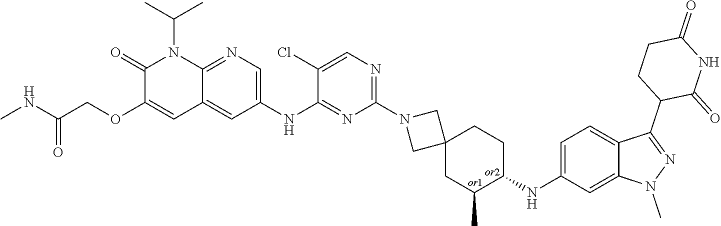 [SMILES: CNC(=O)COc1cc2cc(Nc3nc(N4CC5(CC[C@H](Nc6ccc7c(C8CCC(=O)NC8=O)nn(C)c7c6)[C@@H](C)C5)C4)ncc3Cl)cnc2n(C(C)C)c1=O] |
| 180d | 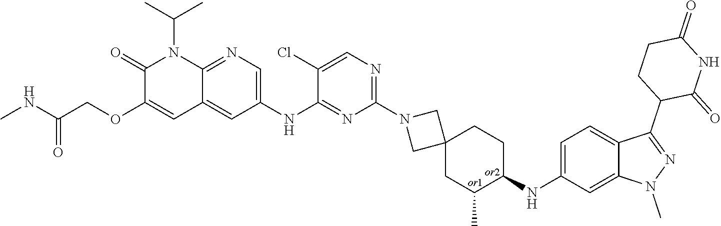 [SMILES: CNC(=O)COc1cc2cc(Nc3nc(N4CC5(CC[C@@H](Nc6ccc7c(C8CCC(=O)NC8=O)nn(C)c7c6)[C@H](C)C5)C4)ncc3Cl)cnc2n(C(C)C)c1=O] |
| 181 | 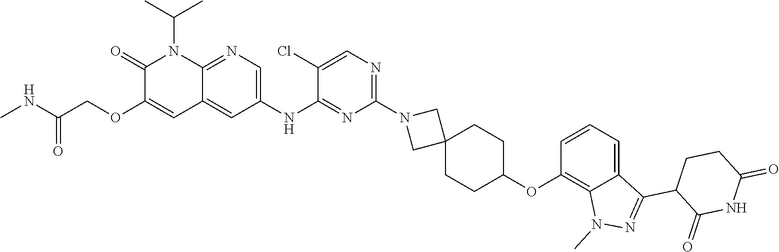 [SMILES: CNC(=O)COc1cc2cc(Nc3nc(N4CC5(CCC(Oc6cccc7c(C8CCC(=O)NC8=O)nn(C)c67)CC5)C4)ncc3Cl)cnc2n(C(C)C)c1=O] |
| 182 | 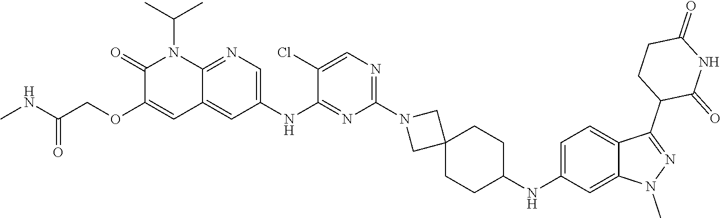 [SMILES: CNC(=O)COc1cc2cc(Nc3nc(N4CC5(CCC(Nc6ccc7c(C8CCC(=O)NC8=O)nn(C)c7c6)CC5)C4)ncc3Cl)cnc2n(C(C)C)c1=O] |
| 183 | 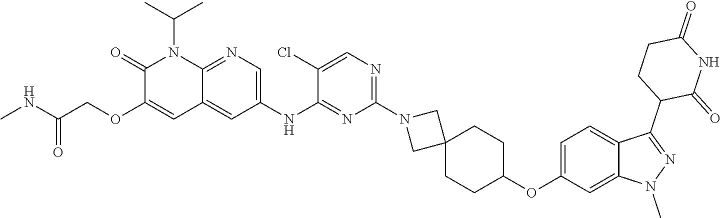 [SMILES: CNC(=O)COc1cc2cc(Nc3nc(N4CC5(CCC(Oc6ccc7c(C8CCC(=O)NC8=O)nn(C)c7c6)CC5)C4)ncc3Cl)cnc2n(C(C)C)c1=O] |
| 184 | 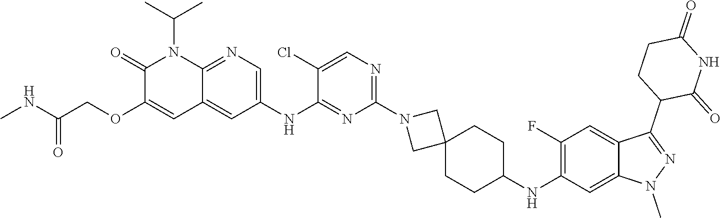 [SMILES: CNC(=O)COc1cc2cc(Nc3nc(N4CC5(CCC(Nc6cc7c(cc6F)c(C6CCC(=O)NC6=O)nn7C)CC5)C4)ncc3Cl)cnc2n(C(C)C)c1=O] |
| 185 | 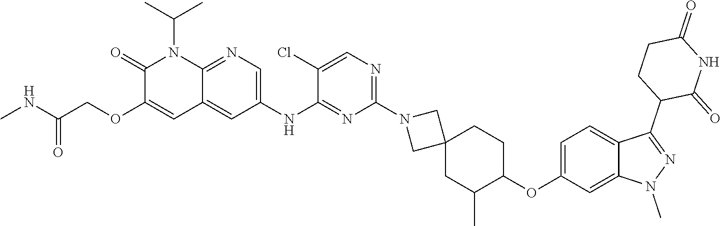 [SMILES: CNC(=O)COc1cc2cc(Nc3nc(N4CC5(CCC(Oc6ccc7c(C8CCC(=O)NC8=O)nn(C)c7c6)C(C)C5)C4)ncc3Cl)cnc2n(C(C)C)c1=O] |
| 185a | 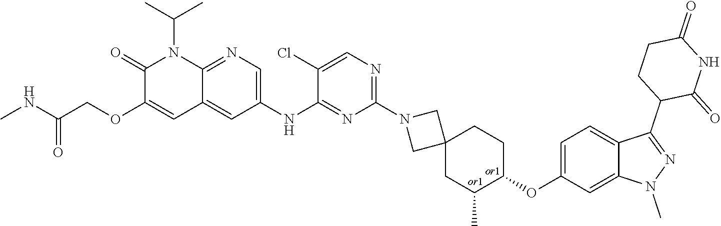 [SMILES: CNC(=O)COc1cc2cc(Nc3nc(N4CC5(CC[C@H](Oc6ccc7c(C8CCC(=O)NC8=O)nn(C)c7c6)[C@H](C)C5)C4)ncc3Cl)cnc2n(C(C)C)c1=O] |
| 185b | 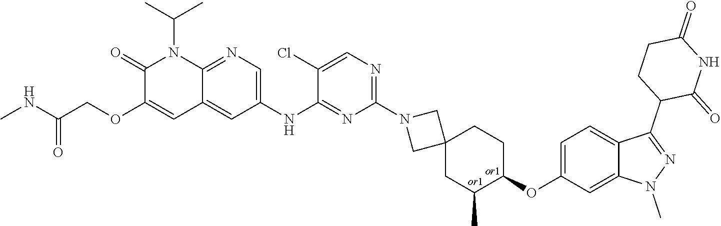 [SMILES: CNC(=O)COc1cc2cc(Nc3nc(N4CC5(CC[C@@H](Oc6ccc7c(C8CCC(=O)NC8=O)nn(C)c7c6)[C@@H](C)C5)C4)ncc3Cl)cnc2n(C(C)C)c1=O] |
| 185c | 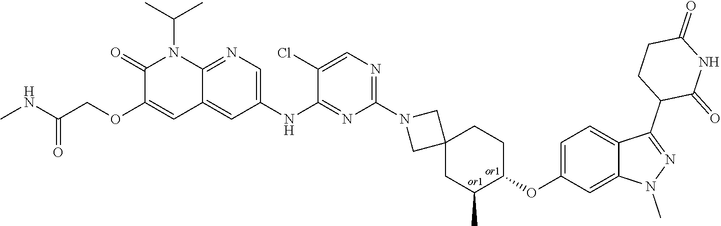 [SMILES: CNC(=O)COc1cc2cc(Nc3nc(N4CC5(CC[C@H](Oc6ccc7c(C8CCC(=O)NC8=O)nn(C)c7c6)[C@@H](C)C5)C4)ncc3Cl)cnc2n(C(C)C)c1=O] |
| 185d | 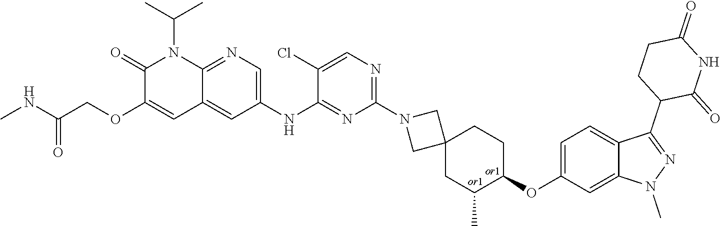 [SMILES: CNC(=O)COc1cc2cc(Nc3nc(N4CC5(CC[C@@H](Oc6ccc7c(C8CCC(=O)NC8=O)nn(C)c7c6)[C@H](C)C5)C4)ncc3Cl)cnc2n(C(C)C)c1=O] |
| 186 | 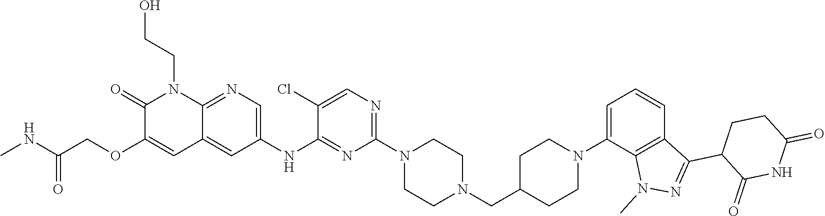 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)cnc2n(CCO)c1=O] |
| 187 | 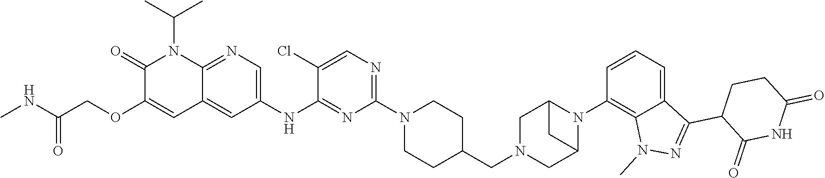 [SMILES: CNC(=O)COc1cc2cc(Nc3nc(N4CCC(CN5CC6CC(C5)N6c5cccc6c(C7CCC(=O)NC7=O)nn(C)c56)CC4)ncc3Cl)cnc2n(C(C)C)c1=O] |
| 188 | 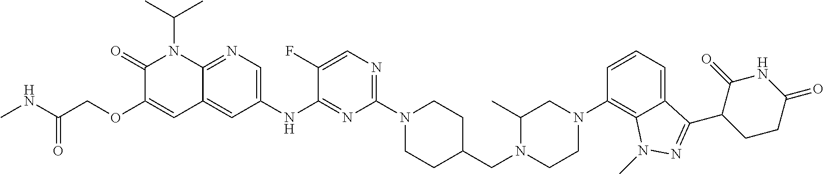 [SMILES: CNC(=O)COc1cc2cc(Nc3nc(N4CCC(CN5CCN(c6cccc7c(C8CCC(=O)NC8=O)nn(C)c67)CC5C)CC4)ncc3F)cnc2n(C(C)C)c1=O] |
| 188a | 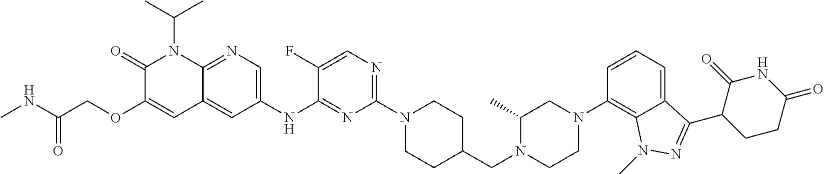 [SMILES: CNC(=O)COc1cc2cc(Nc3nc(N4CCC(CN5CCN(c6cccc7c(C8CCC(=O)NC8=O)nn(C)c67)C[C@H]5C)CC4)ncc3F)cnc2n(C(C)C)c1=O] |
| 188b | 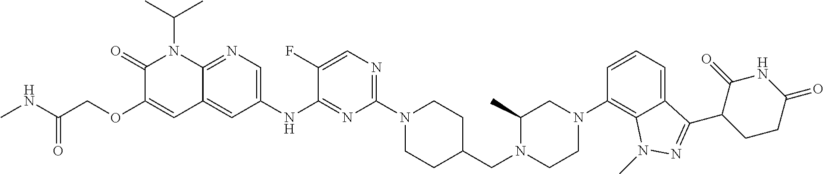 [SMILES: CNC(=O)COc1cc2cc(Nc3nc(N4CCC(CN5CCN(c6cccc7c(C8CCC(=O)NC8=O)nn(C)c67)C[C@@H]5C)CC4)ncc3F)cnc2n(C(C)C)c1=O] |
| 189 | 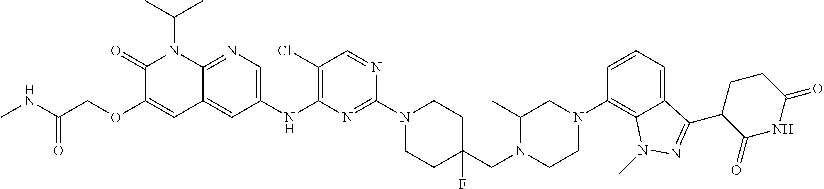 [SMILES: CNC(=O)COc1cc2cc(Nc3nc(N4CCC(F)(CN5CCN(c6cccc7c(C8CCC(=O)NC8=O)nn(C)c67)CC5C)CC4)ncc3Cl)cnc2n(C(C)C)c1=O] |
| 189a | 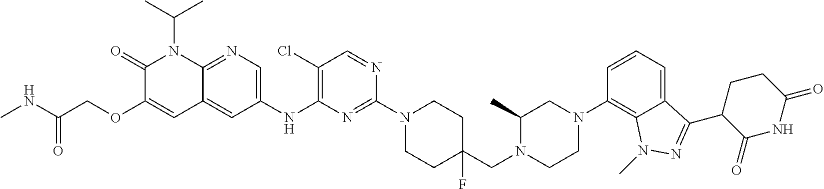 [SMILES: CNC(=O)COc1cc2cc(Nc3nc(N4CCC(F)(CN5CCN(c6cccc7c(C8CCC(=O)NC8=O)nn(C)c67)C[C@@H]5C)CC4)ncc3Cl)cnc2n(C(C)C)c1=O] |
| 190 | 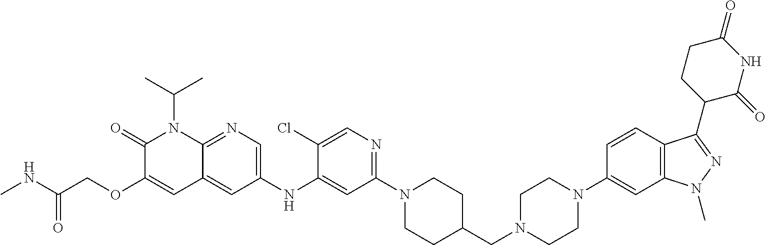 [SMILES: CNC(=O)COc1cc2cc(Nc3cc(N4CCC(CN5CCN(c6ccc7c(C8CCC(=O)NC8=O)nn(C)c7c6)CC5)CC4)ncc3Cl)cnc2n(C(C)C)c1=O] |
| 191 | 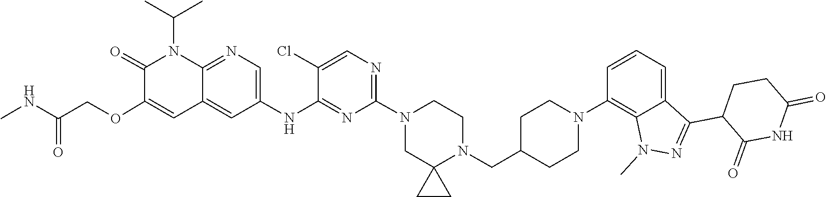 [SMILES: CNC(=O)COc1cc2cc(Nc3nc(N4CCN(CC5CCN(c6cccc7c(C8CCC(=O)NC8=O)nn(C)c67)CC5)C5(CC5)C4)ncc3Cl)cnc2n(C(C)C)c1=O] |
| 192 | 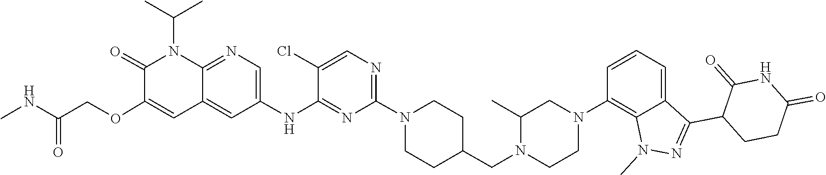 [SMILES: CNC(=O)COc1cc2cc(Nc3nc(N4CCC(CN5CCN(c6cccc7c(C8CCC(=O)NC8=O)nn(C)c67)CC5C)CC4)ncc3Cl)cnc2n(C(C)C)c1=O] |
| 192a | 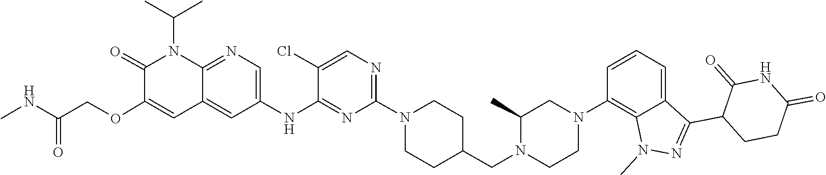 [SMILES: CNC(=O)COc1cc2cc(Nc3nc(N4CCC(CN5CCN(c6cccc7c(C8CCC(=O)NC8=O)nn(C)c67)C[C@@H]5C)CC4)ncc3Cl)cnc2n(C(C)C)c1=O] |
| 192b | 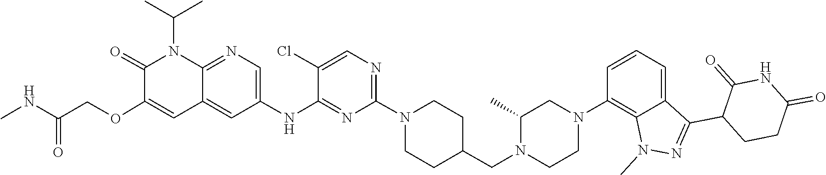 [SMILES: CNC(=O)COc1cc2cc(Nc3nc(N4CCC(CN5CCN(c6cccc7c(C8CCC(=O)NC8=O)nn(C)c67)C[C@H]5C)CC4)ncc3Cl)cnc2n(C(C)C)c1=O] |
| 193 | 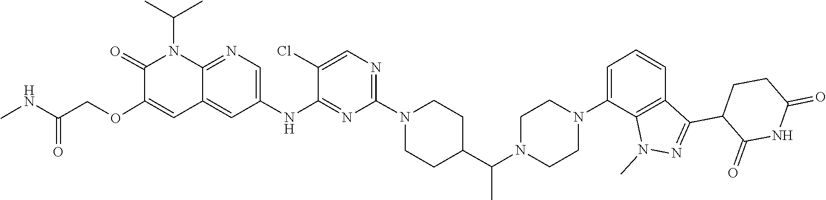 [SMILES: CNC(=O)COc1cc2cc(Nc3nc(N4CCC(C(C)N5CCN(c6cccc7c(C8CCC(=O)NC8=O)nn(C)c67)CC5)CC4)ncc3Cl)cnc2n(C(C)C)c1=O] |
| 193a | 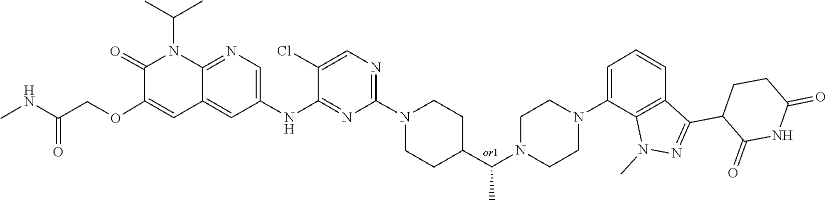 [SMILES: CNC(=O)COc1cc2cc(Nc3nc(N4CCC([C@@H](C)N5CCN(c6cccc7c(C8CCC(=O)NC8=O)nn(C)c67)CC5)CC4)ncc3Cl)cnc2n(C(C)C)c1=O] |
| 193b | 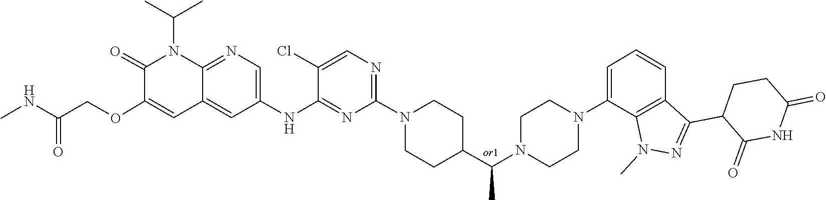 [SMILES: CNC(=O)COc1cc2cc(Nc3nc(N4CCC([C@H](C)N5CCN(c6cccc7c(C8CCC(=O)NC8=O)nn(C)c67)CC5)CC4)ncc3Cl)cnc2n(C(C)C)c1=O] |
| 194 | 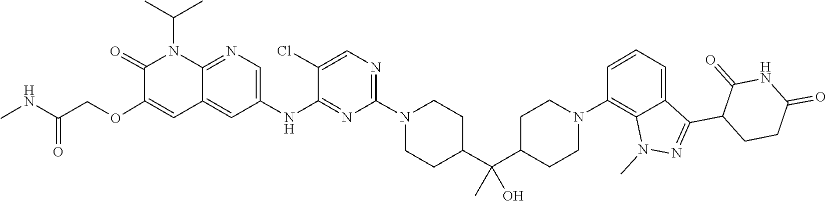 [SMILES: CNC(=O)COc1cc2cc(Nc3nc(N4CCC(C(C)(O)C5CCN(c6cccc7c(C8CCC(=O)NC8=O)nn(C)c67)CC5)CC4)ncc3Cl)cnc2n(C(C)C)c1=O] |
| 194a | 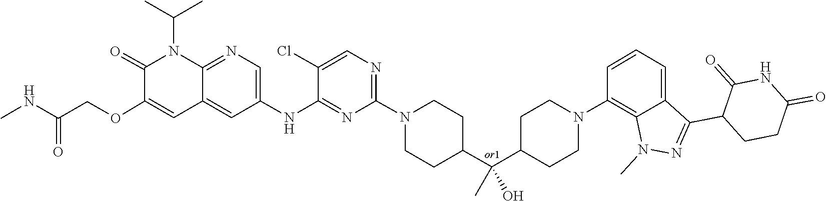 [SMILES: CNC(=O)COc1cc2cc(Nc3nc(N4CCC([C@@](C)(O)C5CCN(c6cccc7c(C8CCC(=O)NC8=O)nn(C)c67)CC5)CC4)ncc3Cl)cnc2n(C(C)C)c1=O] |
| 194b | 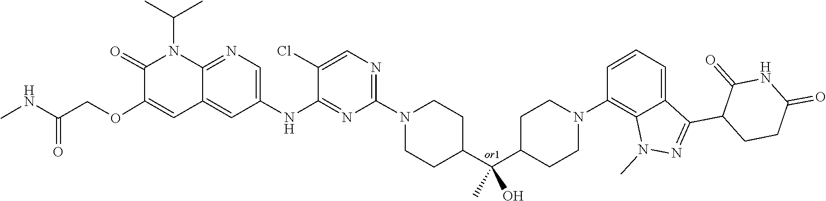 [SMILES: CNC(=O)COc1cc2cc(Nc3nc(N4CCC([C@](C)(O)C5CCN(c6cccc7c(C8CCC(=O)NC8=O)nn(C)c67)CC5)CC4)ncc3Cl)cnc2n(C(C)C)c1=O] |
| 195 | 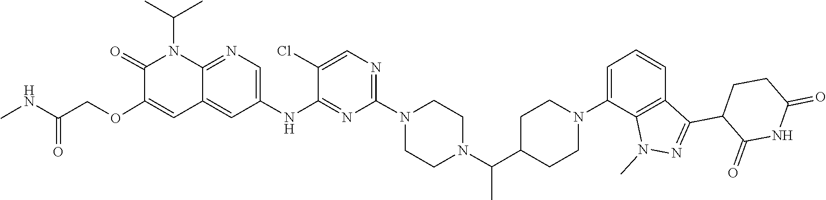 [SMILES: CNC(=O)COc1cc2cc(Nc3nc(N4CCN(C(C)C5CCN(c6cccc7c(C8CCC(=O)NC8=O)nn(C)c67)CC5)CC4)ncc3Cl)cnc2n(C(C)C)c1=O] |
| 195a | 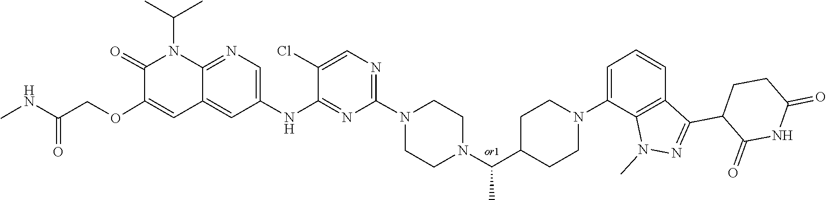 [SMILES: CNC(=O)COc1cc2cc(Nc3nc(N4CCN([C@@H](C)C5CCN(c6cccc7c(C8CCC(=O)NC8=O)nn(C)c67)CC5)CC4)ncc3Cl)cnc2n(C(C)C)c1=O] |
| 195b | 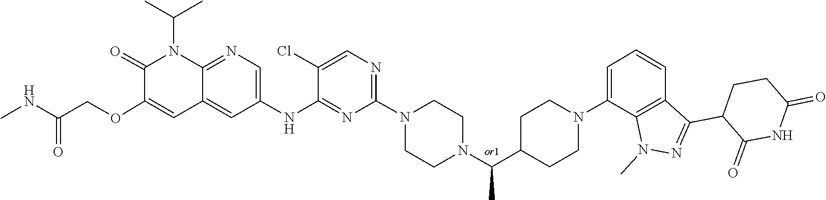 [SMILES: CNC(=O)COc1cc2cc(Nc3nc(N4CCN([C@H](C)C5CCN(c6cccc7c(C8CCC(=O)NC8=O)nn(C)c67)CC5)CC4)ncc3Cl)cnc2n(C(C)C)c1=O] |
| 196 | 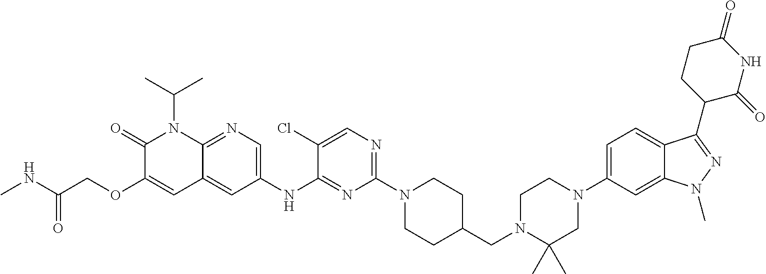 [SMILES: CNC(=O)COc1cc2cc(Nc3nc(N4CCC(CN5CCN(c6ccc7c(C8CCC(=O)NC8=O)nn(C)c7c6)CC5(C)C)CC4)ncc3Cl)cnc2n(C(C)C)c1=O] |
| 197 | 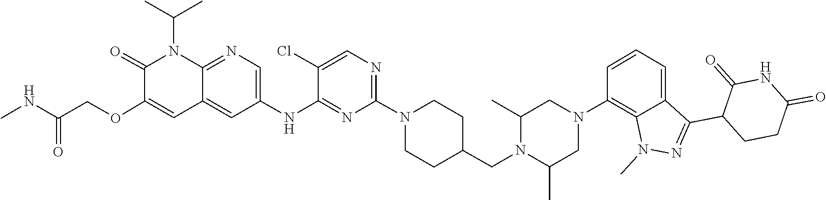 [SMILES: CNC(=O)COc1cc2cc(Nc3nc(N4CCC(CN5C(C)CN(c6cccc7c(C8CCC(=O)NC8=O)nn(C)c67)CC5C)CC4)ncc3Cl)cnc2n(C(C)C)c1=O] |
| 197a | 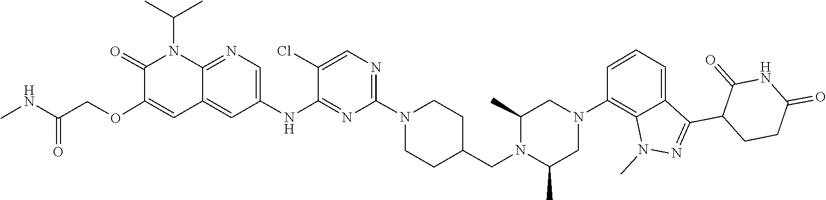 [SMILES: CNC(=O)COc1cc2cc(Nc3nc(N4CCC(CN5[C@H](C)CN(c6cccc7c(C8CCC(=O)NC8=O)nn(C)c67)C[C@@H]5C)CC4)ncc3Cl)cnc2n(C(C)C)c1=O] |
| 197b | 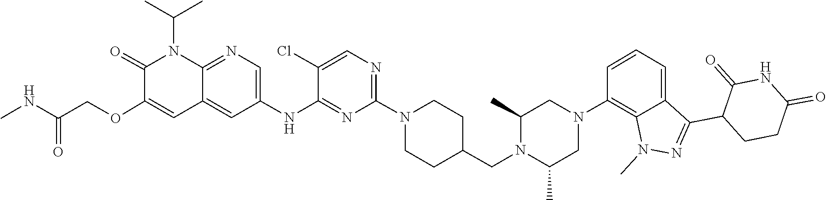 [SMILES: CNC(=O)COc1cc2cc(Nc3nc(N4CCC(CN5[C@@H](C)CN(c6cccc7c(C8CCC(=O)NC8=O)nn(C)c67)C[C@@H]5C)CC4)ncc3Cl)cnc2n(C(C)C)c1=O] |
| 198 | 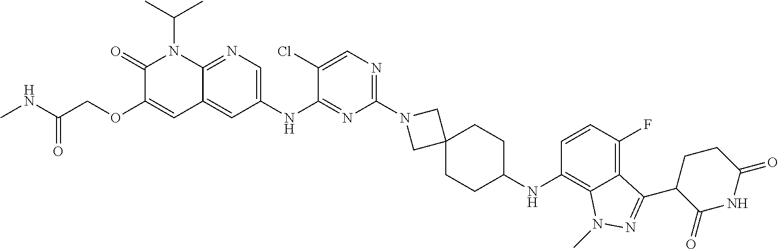 [SMILES: CNC(=O)COc1cc2cc(Nc3nc(N4CC5(CCC(Nc6ccc(F)c7c(C8CCC(=O)NC8=O)nn(C)c67)CC5)C4)ncc3Cl)cnc2n(C(C)C)c1=O] |
| 199 | 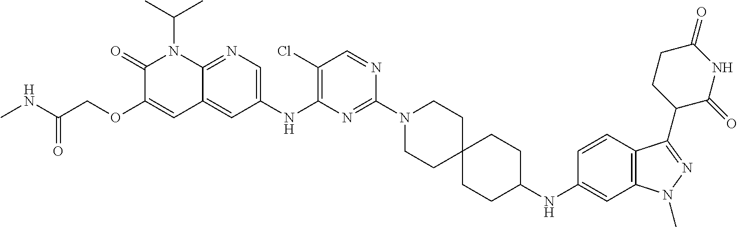 [SMILES: CNC(=O)COc1cc2cc(Nc3nc(N4CCC5(CCC(Nc6ccc7c(C8CCC(=O)NC8=O)nn(C)c7c6)CC5)CC4)ncc3Cl)cnc2n(C(C)C)c1=O] |
| 200 | 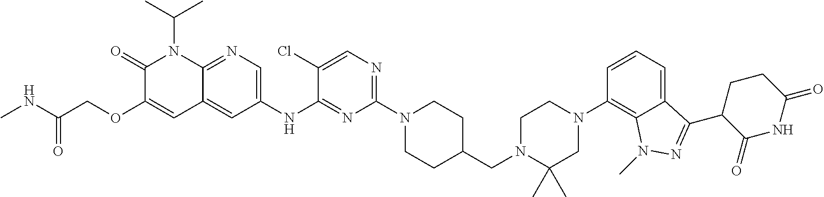 [SMILES: CNC(=O)COc1cc2cc(Nc3nc(N4CCC(CN5CCN(c6cccc7c(C8CCC(=O)NC8=O)nn(C)c67)CC5(C)C)CC4)ncc3Cl)cnc2n(C(C)C)c1=O] |
| 201 | 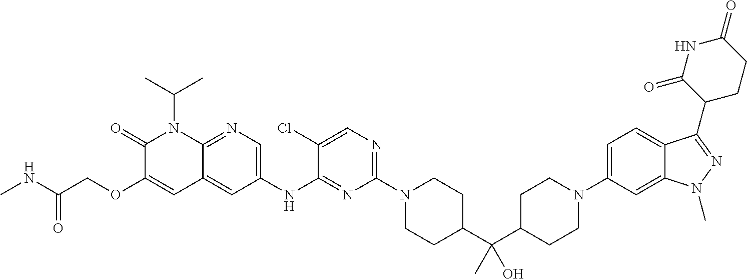 [SMILES: CNC(=O)COc1cc2cc(Nc3nc(N4CCC(C(C)(O)C5CCN(c6ccc7c(C8CCC(=O)NC8=O)nn(C)c7c6)CC5)CC4)ncc3Cl)cnc2n(C(C)C)c1=O] |
| 201a | 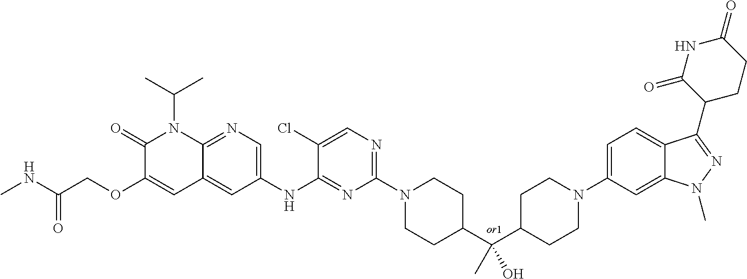 [SMILES: CNC(=O)COc1cc2cc(Nc3nc(N4CCC([C@@](C)(O)C5CCN(c6ccc7c(C8CCC(=O)NC8=O)nn(C)c7c6)CC5)CC4)ncc3Cl)cnc2n(C(C)C)c1=O] |
| 201b | 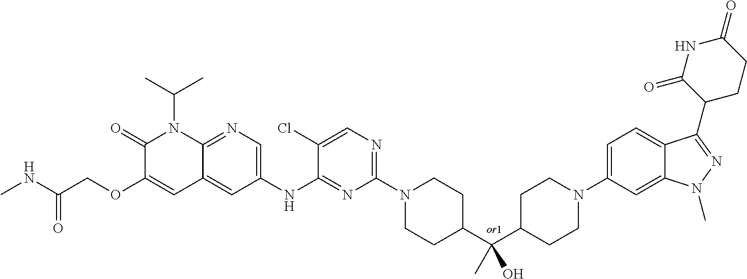 [SMILES: CNC(=O)COc1cc2cc(Nc3nc(N4CCC([C@](C)(O)C5CCN(c6ccc7c(C8CCC(=O)NC8=O)nn(C)c7c6)CC5)CC4)ncc3Cl)cnc2n(C(C)C)c1=O] |
| 202 | 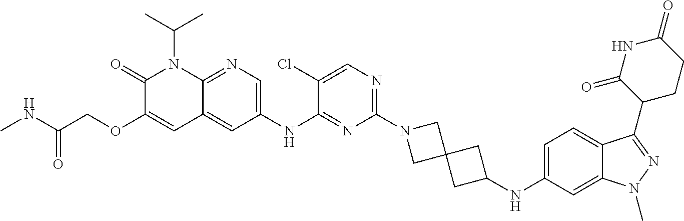 [SMILES: CNC(=O)COc1cc2cc(Nc3nc(N4CC5(CC(Nc6ccc7c(C8CCC(=O)NC8=O)nn(C)c7c6)C5)C4)ncc3Cl)cnc2n(C(C)C)c1=O] |
| 203 | 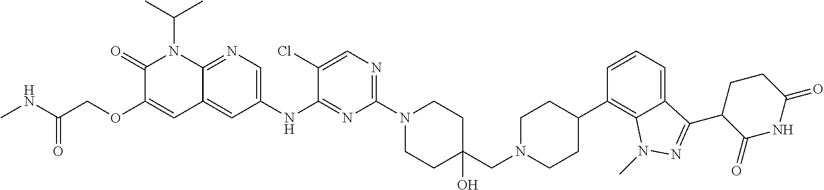 [SMILES: CNC(=O)COc1cc2cc(Nc3nc(N4CCC(O)(CN5CCC(c6cccc7c(C8CCC(=O)NC8=O)nn(C)c67)CC5)CC4)ncc3Cl)cnc2n(C(C)C)c1=O] |
| 204 | 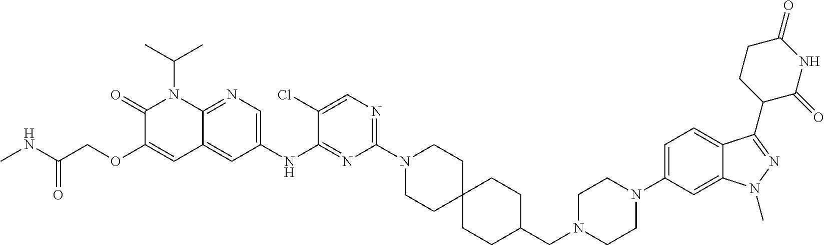 [SMILES: CNC(=O)COc1cc2cc(Nc3nc(N4CCC5(CCC(CN6CCN(c7ccc8c(C9CCC(=O)NC9=O)nn(C)c8c7)CC6)CC5)CC4)ncc3Cl)cnc2n(C(C)C)c1=O] |
| 205 | 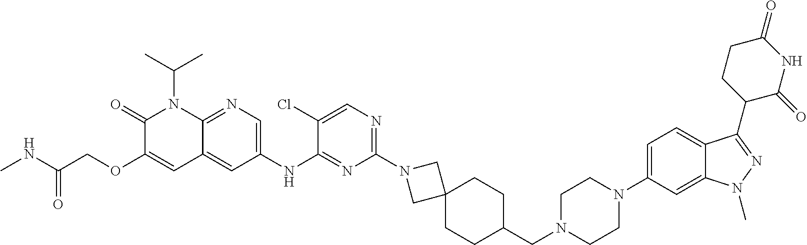 [SMILES: CNC(=O)COc1cc2cc(Nc3nc(N4CC5(CCC(CN6CCN(c7ccc8c(C9CCC(=O)NC9=O)nn(C)c8c7)CC6)CC5)C4)ncc3Cl)cnc2n(C(C)C)c1=O] |
| 206 | 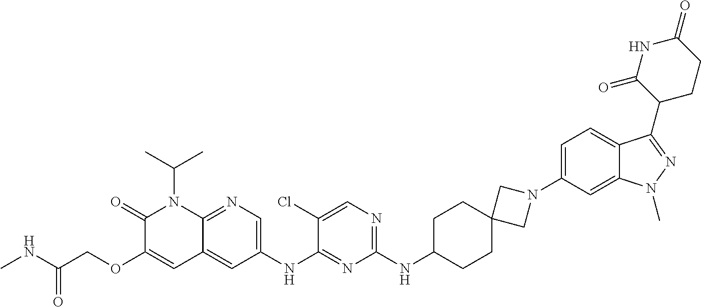 [SMILES: CNC(=O)COc1cc2cc(Nc3nc(NC4CCC5(CC4)CN(c4ccc6c(C7CCC(=O)NC7=O)nn(C)c6c4)C5)ncc3Cl)cnc2n(C(C)C)c1=O] |
| 207 | 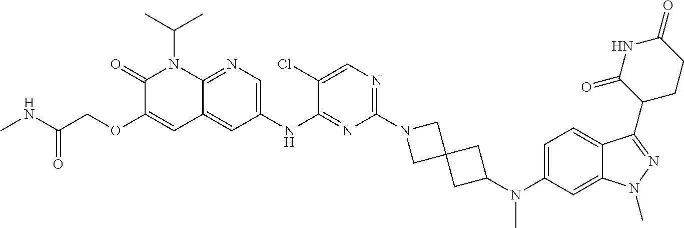 [SMILES: CNC(=O)COc1cc2cc(Nc3nc(N4CC5(CC(N(C)c6ccc7c(C8CCC(=O)NC8=O)nn(C)c7c6)C5)C4)ncc3Cl)cnc2n(C(C)C)c1=O] |
| 208 | 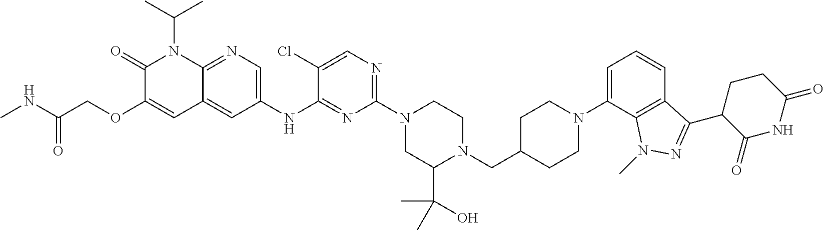 [SMILES: CNC(=O)COc1cc2cc(Nc3nc(N4CCN(CC5CCN(c6cccc7c(C8CCC(=O)NC8=O)nn(C)c67)CC5)C(C(C)(C)O)C4)ncc3Cl)cnc2n(C(C)C)c1=O] |
| 208a | 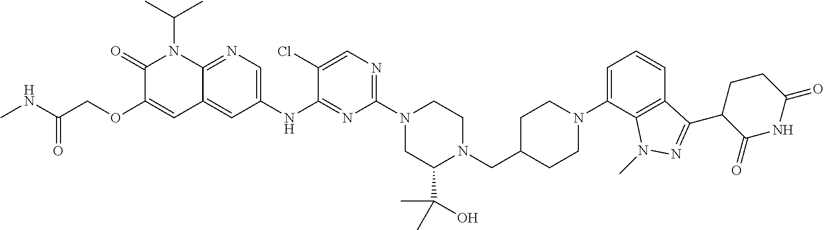 [SMILES: CNC(=O)COc1cc2cc(Nc3nc(N4CCN(CC5CCN(c6cccc7c(C8CCC(=O)NC8=O)nn(C)c67)CC5)[C@H](C(C)(C)O)C4)ncc3Cl)cnc2n(C(C)C)c1=O] |
| 208b | 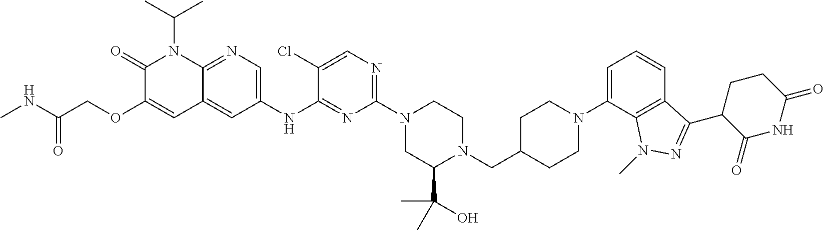 [SMILES: CNC(=O)COc1cc2cc(Nc3nc(N4CCN(CC5CCN(c6cccc7c(C8CCC(=O)NC8=O)nn(C)c67)CC5)[C@@H](C(C)(C)O)C4)ncc3Cl)cnc2n(C(C)C)c1=O] |
| 209 | 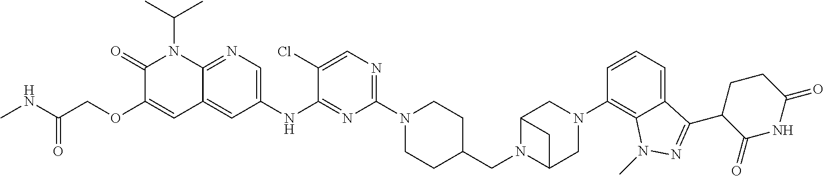 [SMILES: CNC(=O)COc1cc2cc(Nc3nc(N4CCC(CN5C6CC5CN(c5cccc7c(C8CCC(=O)NC8=O)nn(C)c57)C6)CC4)ncc3Cl)cnc2n(C(C)C)c1=O] |
| 210 | 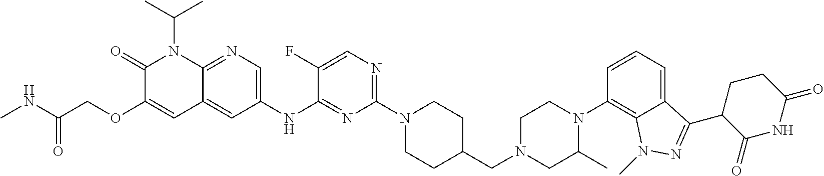 [SMILES: CNC(=O)COc1cc2cc(Nc3nc(N4CCC(CN5CCN(c6cccc7c(C8CCC(=O)NC8=O)nn(C)c67)C(C)C5)CC4)ncc3F)cnc2n(C(C)C)c1=O] |
| 210a | 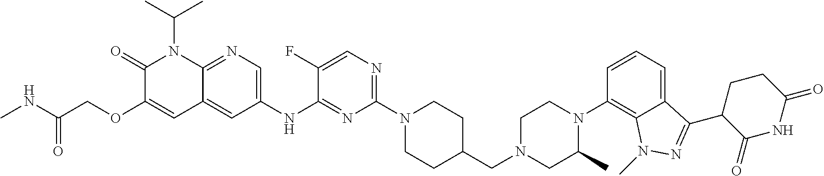 [SMILES: CNC(=O)COc1cc2cc(Nc3nc(N4CCC(CN5CCN(c6cccc7c(C8CCC(=O)NC8=O)nn(C)c67)[C@@H](C)C5)CC4)ncc3F)cnc2n(C(C)C)c1=O] |
| 210b | 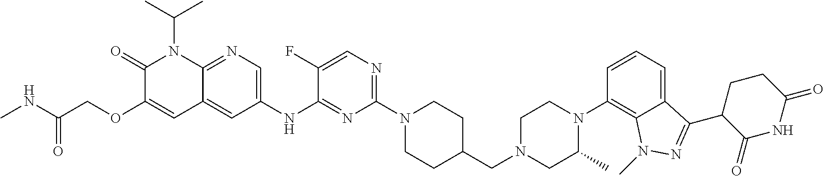 [SMILES: CNC(=O)COc1cc2cc(Nc3nc(N4CCC(CN5CCN(c6cccc7c(C8CCC(=O)NC8=O)nn(C)c67)[C@H](C)C5)CC4)ncc3F)cnc2n(C(C)C)c1=O] |
| 211 | 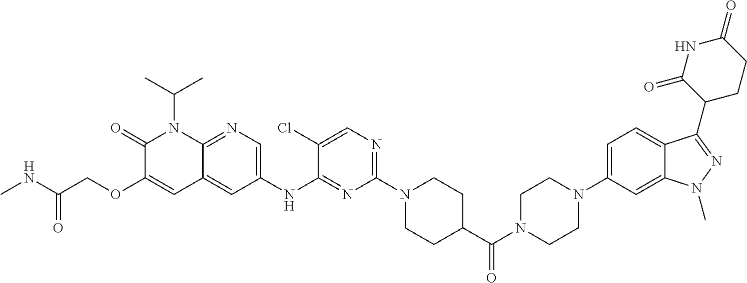 [SMILES: CNC(=O)COc1cc2cc(Nc3nc(N4CCC(C(=O)N5CCN(c6ccc7c(C8CCC(=O)NC8=O)nn(C)c7c6)CC5)CC4)ncc3Cl)cnc2n(C(C)C)c1=O] |
| 212 | 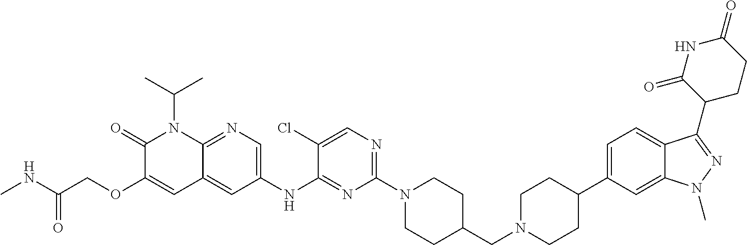 [SMILES: CNC(=O)COc1cc2cc(Nc3nc(N4CCC(CN5CCC(c6ccc7c(C8CCC(=O)NC8=O)nn(C)c7c6)CC5)CC4)ncc3Cl)cnc2n(C(C)C)c1=O] |
| 213 | 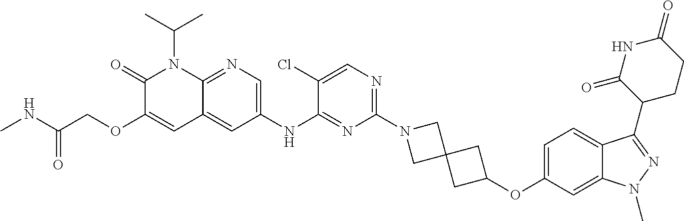 [SMILES: CNC(=O)COc1cc2cc(Nc3nc(N4CC5(CC(Oc6ccc7c(C8CCC(=O)NC8=O)nn(C)c7c6)C5)C4)ncc3Cl)cnc2n(C(C)C)c1=O] |
| 214 | 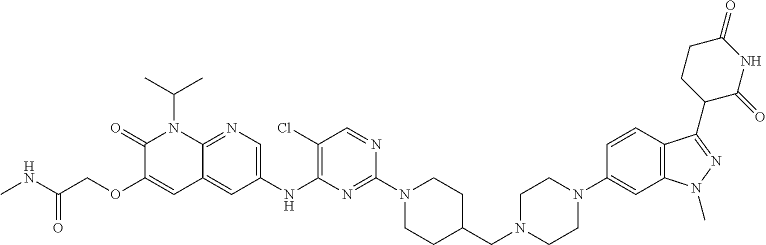 [SMILES: CNC(=O)COc1cc2cc(Nc3nc(N4CCC(CN5CCN(c6ccc7c(C8CCC(=O)NC8=O)nn(C)c7c6)CC5)CC4)ncc3Cl)cnc2n(C(C)C)c1=O] |
| 215 | 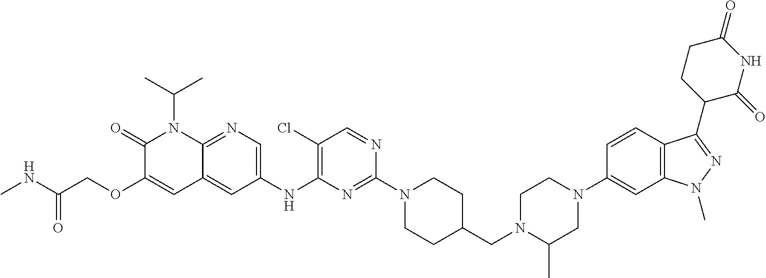 [SMILES: CNC(=O)COc1cc2cc(Nc3nc(N4CCC(CN5CCN(c6ccc7c(C8CCC(=O)NC8=O)nn(C)c7c6)CC5C)CC4)ncc3Cl)cnc2n(C(C)C)c1=O] |
| 215a | 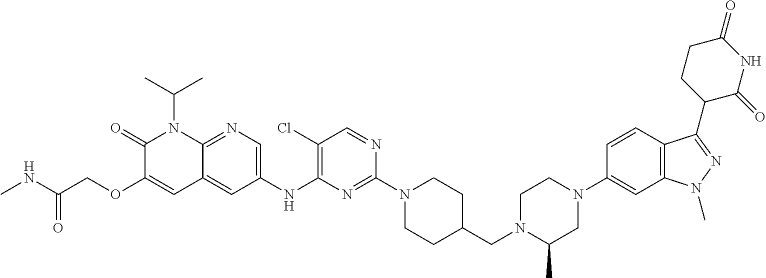 [SMILES: CNC(=O)COc1cc2cc(Nc3nc(N4CCC(CN5CCN(c6ccc7c(C8CCC(=O)NC8=O)nn(C)c7c6)C[C@H]5C)CC4)ncc3Cl)cnc2n(C(C)C)c1=O] |
| 215b | 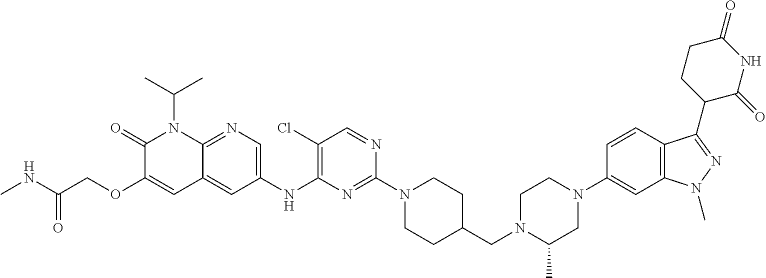 [SMILES: CNC(=O)COc1cc2cc(Nc3nc(N4CCC(CN5CCN(c6ccc7c(C8CCC(=O)NC8=O)nn(C)c7c6)C[C@@H]5C)CC4)ncc3Cl)cnc2n(C(C)C)c1=O] |
| 216 | 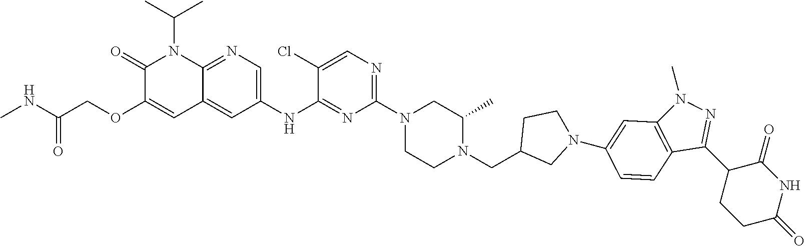 [SMILES: CNC(=O)COc1cc2cc(Nc3nc(N4CCN(CC5CCN(c6ccc7c(C8CCC(=O)NC8=O)nn(C)c7c6)C5)[C@@H](C)C4)ncc3Cl)cnc2n(C(C)C)c1=O] |
| 216a | 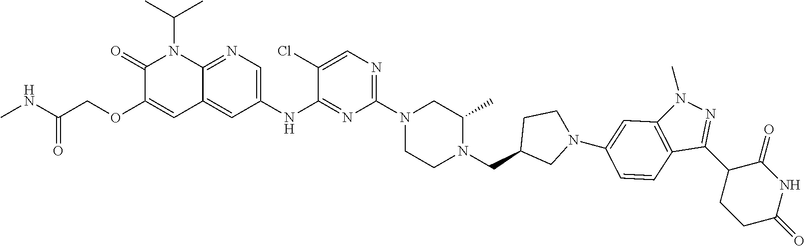 [SMILES: CNC(=O)COc1cc2cc(Nc3nc(N4CCN(C[C@H]5CCN(c6ccc7c(C8CCC(=O)NC8=O)nn(C)c7c6)C5)[C@@H](C)C4)ncc3Cl)cnc2n(C(C)C)c1=O] |
| 216b | 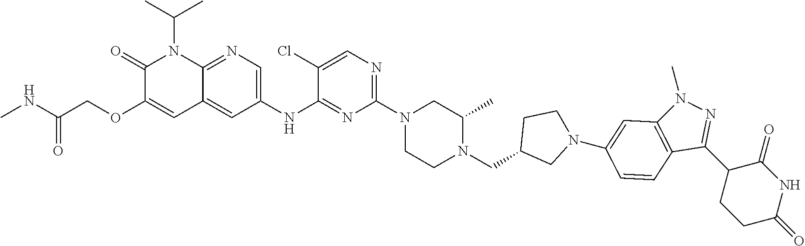 [SMILES: CNC(=O)COc1cc2cc(Nc3nc(N4CCN(C[C@@H]5CCN(c6ccc7c(C8CCC(=O)NC8=O)nn(C)c7c6)C5)[C@@H](C)C4)ncc3Cl)cnc2n(C(C)C)c1=O] |
| 216c | 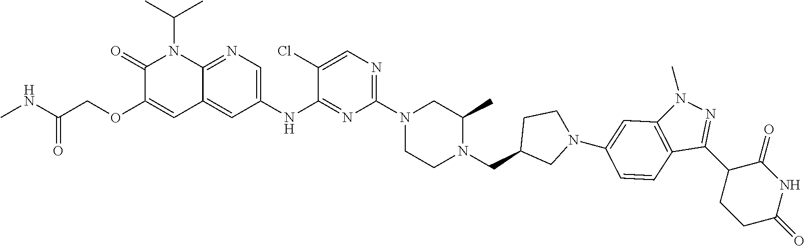 [SMILES: CNC(=O)COc1cc2cc(Nc3nc(N4CCN(C[C@H]5CCN(c6ccc7c(C8CCC(=O)NC8=O)nn(C)c7c6)C5)[C@H](C)C4)ncc3Cl)cnc2n(C(C)C)c1=O] |
| 216d | 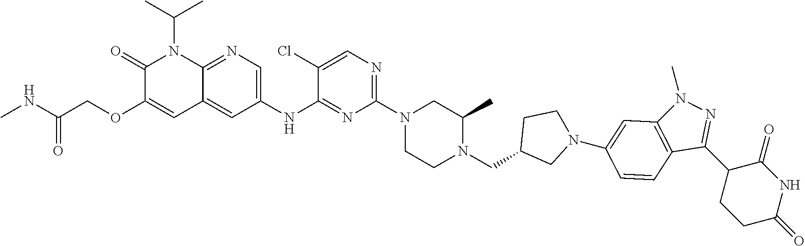 [SMILES: CNC(=O)COc1cc2cc(Nc3nc(N4CCN(C[C@@H]5CCN(c6ccc7c(C8CCC(=O)NC8=O)nn(C)c7c6)C5)[C@H](C)C4)ncc3Cl)cnc2n(C(C)C)c1=O] |
| 217 | 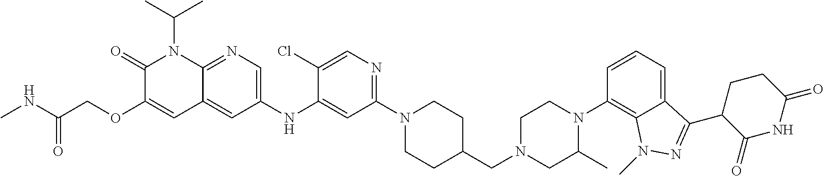 [SMILES: CNC(=O)COc1cc2cc(Nc3cc(N4CCC(CN5CCN(c6cccc7c(C8CCC(=O)NC8=O)nn(C)c67)C(C)C5)CC4)ncc3Cl)cnc2n(C(C)C)c1=O] |
| 217a | 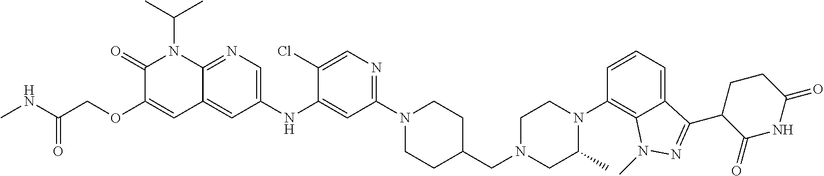 [SMILES: CNC(=O)COc1cc2cc(Nc3cc(N4CCC(CN5CCN(c6cccc7c(C8CCC(=O)NC8=O)nn(C)c67)[C@H](C)C5)CC4)ncc3Cl)cnc2n(C(C)C)c1=O] |
| 217b | 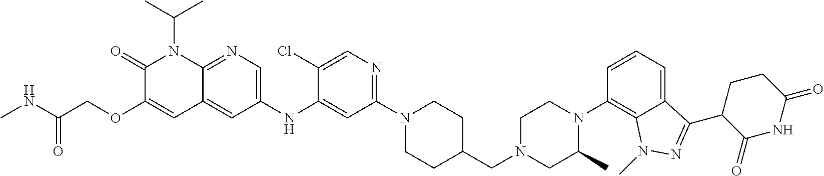 [SMILES: CNC(=O)COc1cc2cc(Nc3cc(N4CCC(CN5CCN(c6cccc7c(C8CCC(=O)NC8=O)nn(C)c67)[C@@H](C)C5)CC4)ncc3Cl)cnc2n(C(C)C)c1=O] |
| 218 | 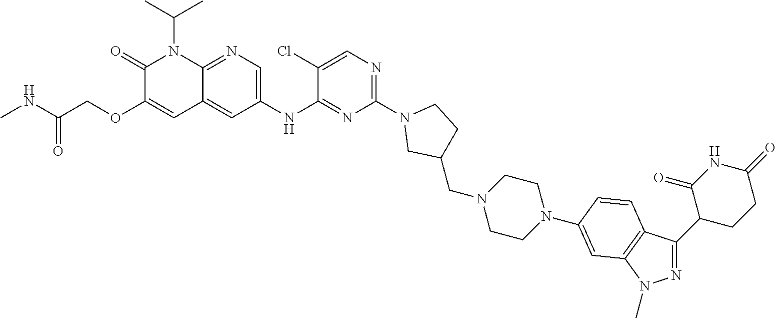 [SMILES: CNC(=O)COc1cc2cc(Nc3nc(N4CCC(CN5CCN(c6ccc7c(C8CCC(=O)NC8=O)nn(C)c7c6)CC5)C4)ncc3Cl)cnc2n(C(C)C)c1=O] |
| 218a | 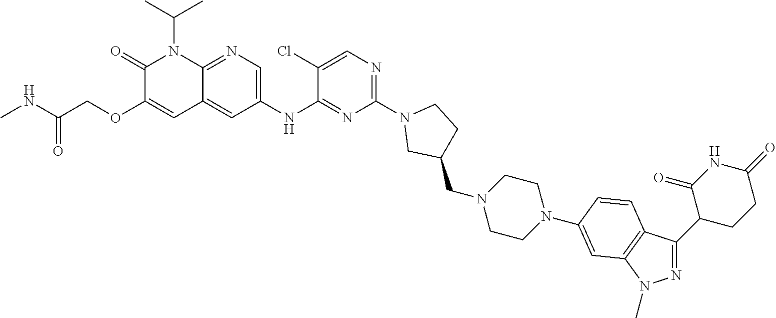 [SMILES: CNC(=O)COc1cc2cc(Nc3nc(N4CC[C@@H](CN5CCN(c6ccc7c(C8CCC(=O)NC8=O)nn(C)c7c6)CC5)C4)ncc3Cl)cnc2n(C(C)C)c1=O] |
| 218b | 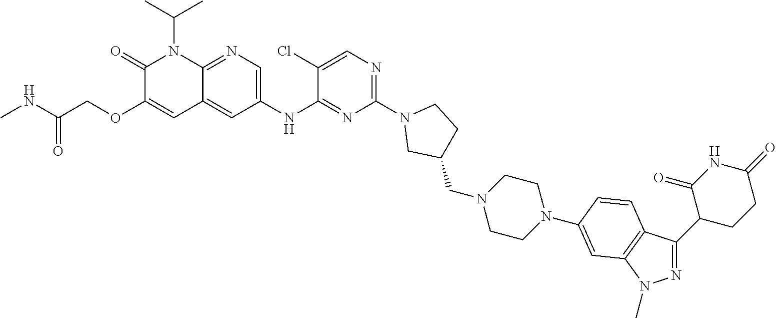 [SMILES: CNC(=O)COc1cc2cc(Nc3nc(N4CC[C@H](CN5CCN(c6ccc7c(C8CCC(=O)NC8=O)nn(C)c7c6)CC5)C4)ncc3Cl)cnc2n(C(C)C)c1=O] |
| 219 | 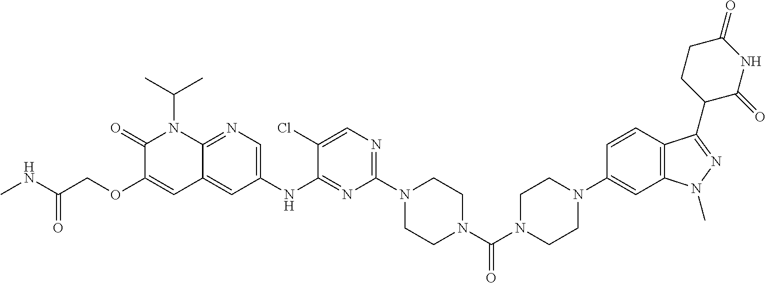 [SMILES: CNC(=O)COc1cc2cc(Nc3nc(N4CCN(C(=O)N5CCN(c6ccc7c(C8CCC(=O)NC8=O)nn(C)c7c6)CC5)CC4)ncc3Cl)cnc2n(C(C)C)c1=O] |
| 220 | 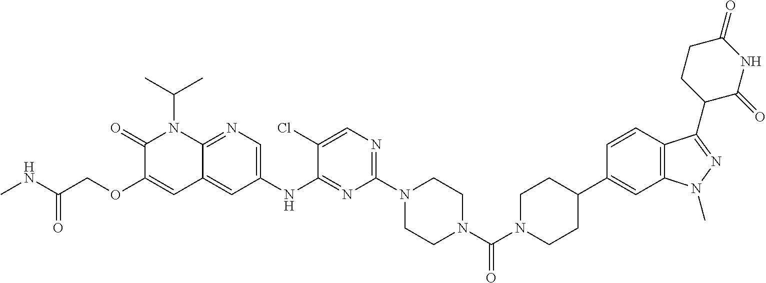 [SMILES: CNC(=O)COc1cc2cc(Nc3nc(N4CCN(C(=O)N5CCC(c6ccc7c(C8CCC(=O)NC8=O)nn(C)c7c6)CC5)CC4)ncc3Cl)cnc2n(C(C)C)c1=O] |
| 221 | 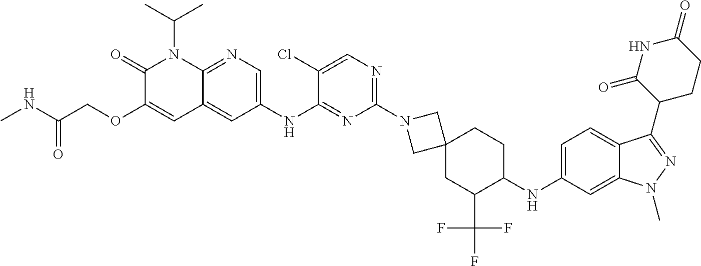 [SMILES: CNC(=O)COc1cc2cc(Nc3nc(N4CC5(CCC(Nc6ccc7c(C8CCC(=O)NC8=O)nn(C)c7c6)C(C(F)(F)F)C5)C4)ncc3Cl)cnc2n(C(C)C)c1=O] |
| 221a | 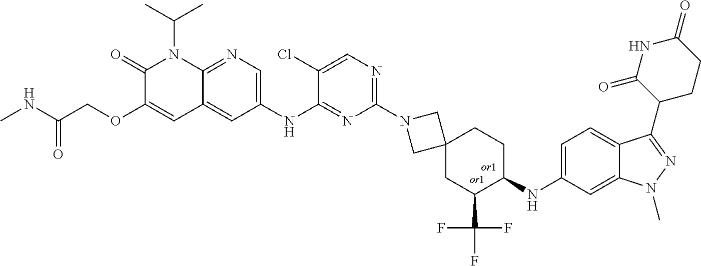 [SMILES: CNC(=O)COc1cc2cc(Nc3nc(N4CC5(CC[C@@H](Nc6ccc7c(C8CCC(=O)NC8=O)nn(C)c7c6)[C@@H](C(F)(F)F)C5)C4)ncc3Cl)cnc2n(C(C)C)c1=O] |
| 221b | 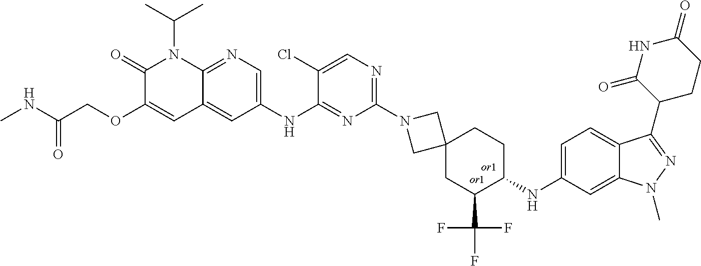 [SMILES: CNC(=O)COc1cc2cc(Nc3nc(N4CC5(CC[C@H](Nc6ccc7c(C8CCC(=O)NC8=O)nn(C)c7c6)[C@@H](C(F)(F)F)C5)C4)ncc3Cl)cnc2n(C(C)C)c1=O] |
| 221c | 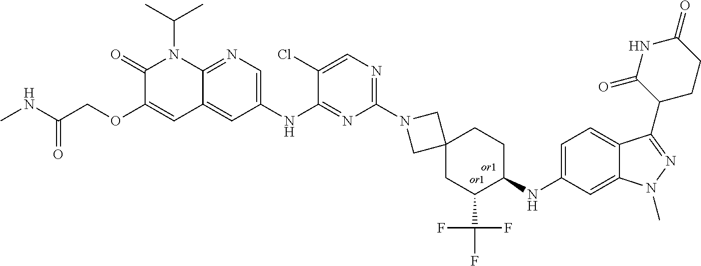 [SMILES: CNC(=O)COc1cc2cc(Nc3nc(N4CC5(CC[C@@H](Nc6ccc7c(C8CCC(=O)NC8=O)nn(C)c7c6)[C@H](C(F)(F)F)C5)C4)ncc3Cl)cnc2n(C(C)C)c1=O] |
| 221d | 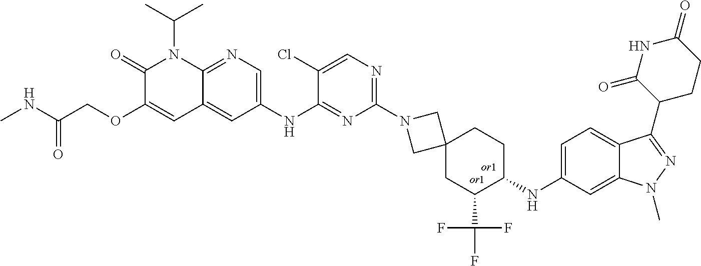 [SMILES: CNC(=O)COc1cc2cc(Nc3nc(N4CC5(CC[C@H](Nc6ccc7c(C8CCC(=O)NC8=O)nn(C)c7c6)[C@H](C(F)(F)F)C5)C4)ncc3Cl)cnc2n(C(C)C)c1=O] |
| 222 | 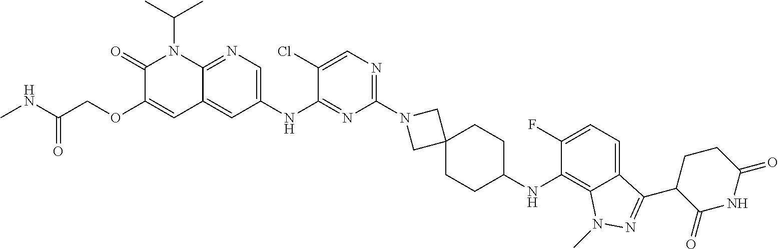 [SMILES: CNC(=O)COc1cc2cc(Nc3nc(N4CC5(CCC(Nc6c(F)ccc7c(C8CCC(=O)NC8=O)nn(C)c67)CC5)C4)ncc3Cl)cnc2n(C(C)C)c1=O] |
| 223 | 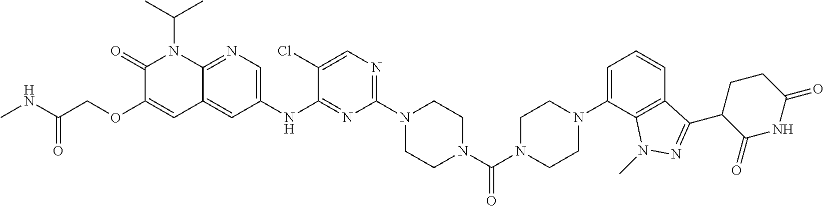 [SMILES: CNC(=O)COc1cc2cc(Nc3nc(N4CCN(C(=O)N5CCN(c6cccc7c(C8CCC(=O)NC8=O)nn(C)c67)CC5)CC4)ncc3Cl)cnc2n(C(C)C)c1=O] |
| 224 | 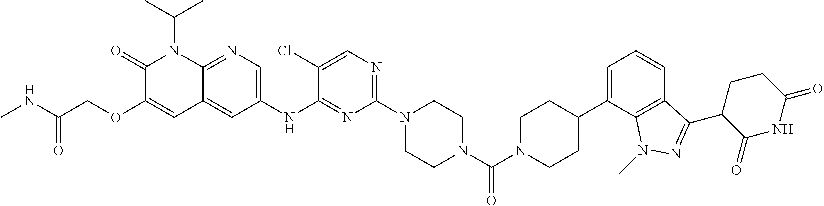 [SMILES: CNC(=O)COc1cc2cc(Nc3nc(N4CCN(C(=O)N5CCC(c6cccc7c(C8CCC(=O)NC8=O)nn(C)c67)CC5)CC4)ncc3Cl)cnc2n(C(C)C)c1=O] |
| 225 | 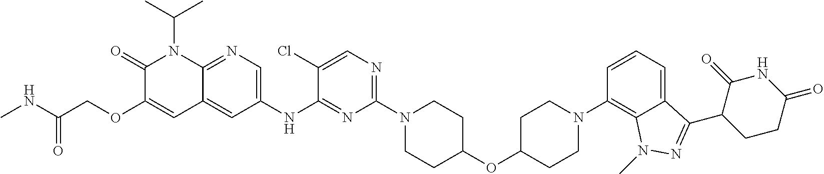 [SMILES: CNC(=O)COc1cc2cc(Nc3nc(N4CCC(OC5CCN(c6cccc7c(C8CCC(=O)NC8=O)nn(C)c67)CC5)CC4)ncc3Cl)cnc2n(C(C)C)c1=O] |
| 226 | 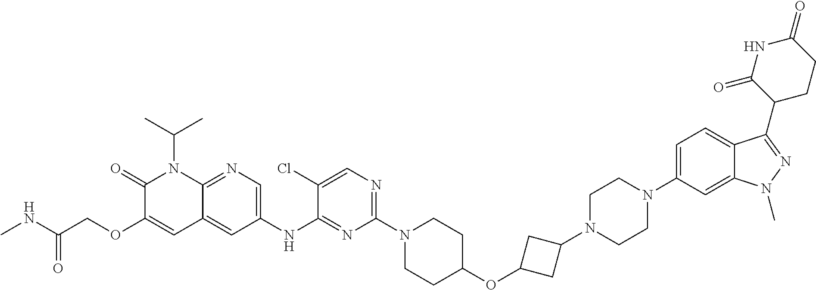 [SMILES: CNC(=O)COc1cc2cc(Nc3nc(N4CCC(OC5CC(N6CCN(c7ccc8c(C9CCC(=O)NC9=O)nn(C)c8c7)CC6)C5)CC4)ncc3Cl)cnc2n(C(C)C)c1=O] |
| 226a | 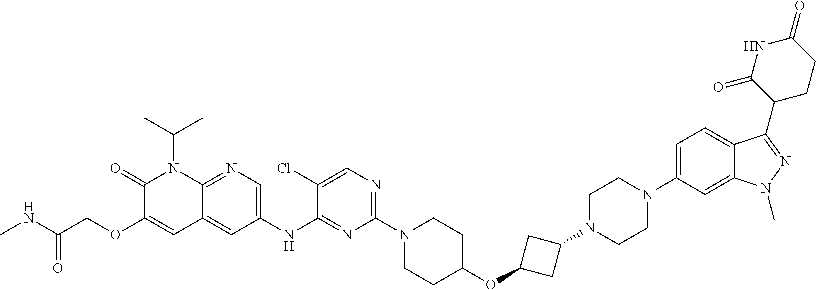 [SMILES: CNC(=O)COc1cc2cc(Nc3nc(N4CCC(O[C@H]5C[C@H](N6CCN(c7ccc8c(C9CCC(=O)NC9=O)nn(C)c8c7)CC6)C5)CC4)ncc3Cl)cnc2n(C(C)C)c1=O] |
| 227 | 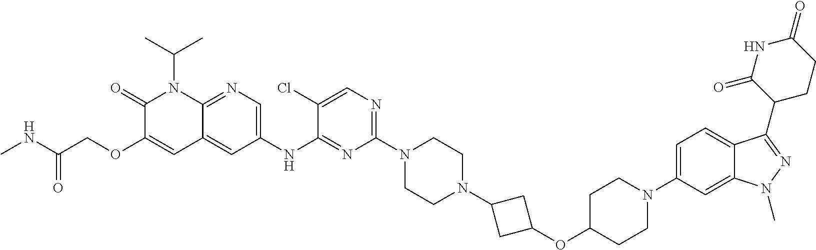 [SMILES: CNC(=O)COc1cc2cc(Nc3nc(N4CCN(C5CC(OC6CCN(c7ccc8c(C9CCC(=O)NC9=O)nn(C)c8c7)CC6)C5)CC4)ncc3Cl)cnc2n(C(C)C)c1=O] |
| 227a | 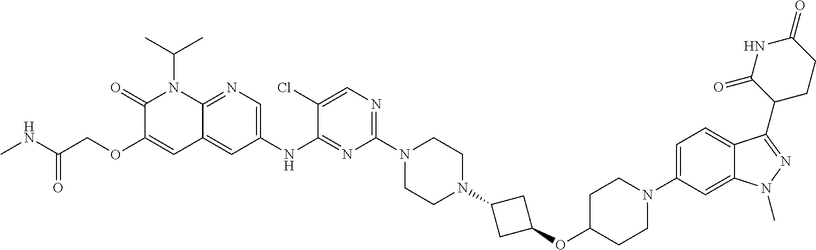 [SMILES: CNC(=O)COc1cc2cc(Nc3nc(N4CCN([C@H]5C[C@H](OC6CCN(c7ccc8c(C9CCC(=O)NC9=O)nn(C)c8c7)CC6)C5)CC4)ncc3Cl)cnc2n(C(C)C)c1=O] |
| 228 | 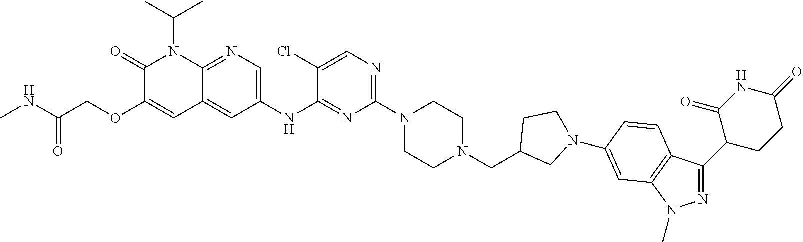 [SMILES: CNC(=O)COc1cc2cc(Nc3nc(N4CCN(CC5CCN(c6ccc7c(C8CCC(=O)NC8=O)nn(C)c7c6)C5)CC4)ncc3Cl)cnc2n(C(C)C)c1=O] |
| 228a | 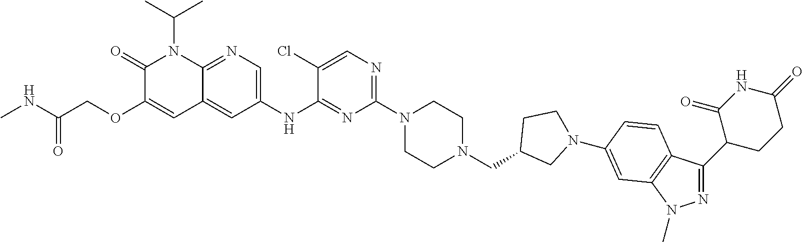 [SMILES: CNC(=O)COc1cc2cc(Nc3nc(N4CCN(C[C@@H]5CCN(c6ccc7c(C8CCC(=O)NC8=O)nn(C)c7c6)C5)CC4)ncc3Cl)cnc2n(C(C)C)c1=O] |
| 228b | 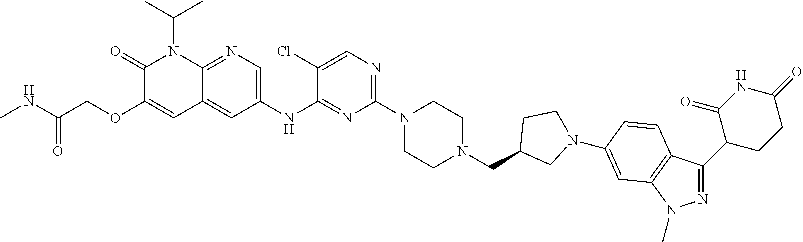 [SMILES: CNC(=O)COc1cc2cc(Nc3nc(N4CCN(C[C@H]5CCN(c6ccc7c(C8CCC(=O)NC8=O)nn(C)c7c6)C5)CC4)ncc3Cl)cnc2n(C(C)C)c1=O] |
| 229 | 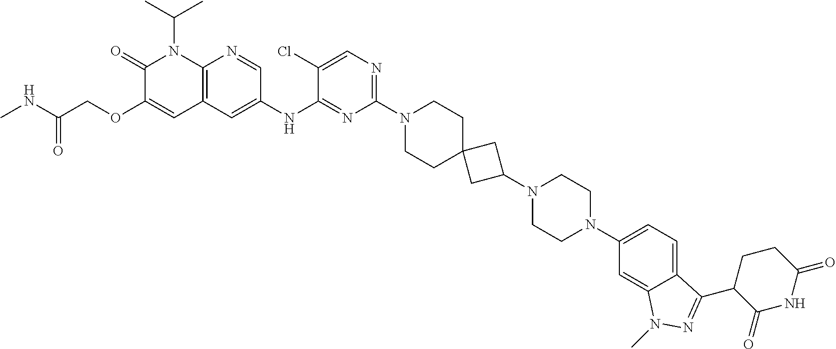 [SMILES: CNC(=O)COc1cc2cc(Nc3nc(N4CCC5(CC4)CC(N4CCN(c6ccc7c(C8CCC(=O)NC8=O)nn(C)c7c6)CC4)C5)ncc3Cl)cnc2n(C(C)C)c1=O] |
| 230 | 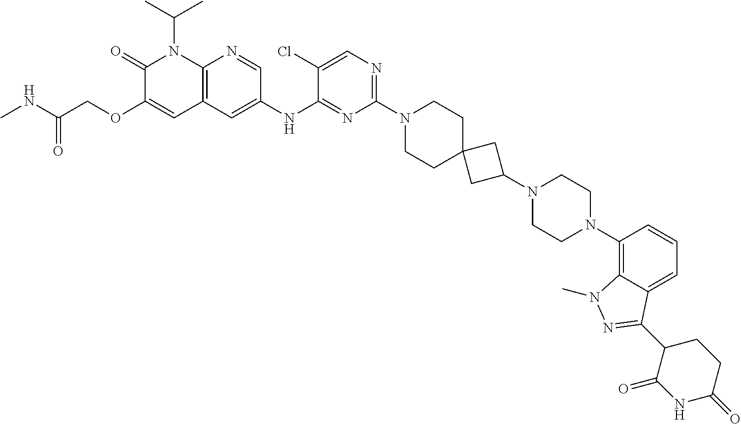 [SMILES: CNC(=O)COc1cc2cc(Nc3nc(N4CCC5(CC4)CC(N4CCN(c6cccc7c(C8CCC(=O)NC8=O)nn(C)c67)CC4)C5)ncc3Cl)cnc2n(C(C)C)c1=O] |
| 231 | 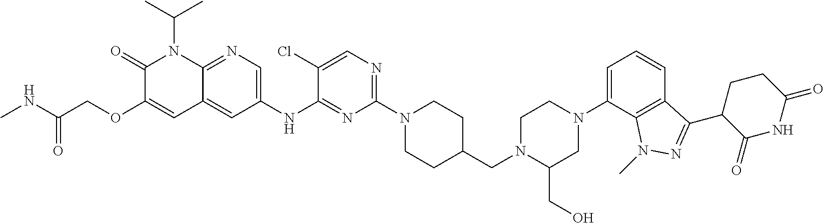 [SMILES: CNC(=O)COc1cc2cc(Nc3nc(N4CCC(CN5CCN(c6cccc7c(C8CCC(=O)NC8=O)nn(C)c67)CC5CO)CC4)ncc3Cl)cnc2n(C(C)C)c1=O] |
| 231a | 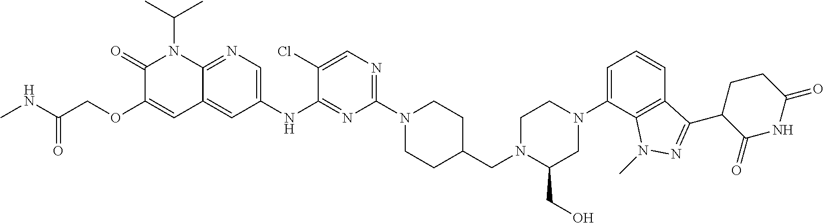 [SMILES: CNC(=O)COc1cc2cc(Nc3nc(N4CCC(CN5CCN(c6cccc7c(C8CCC(=O)NC8=O)nn(C)c67)C[C@H]5CO)CC4)ncc3Cl)cnc2n(C(C)C)c1=O] |
| 231b | 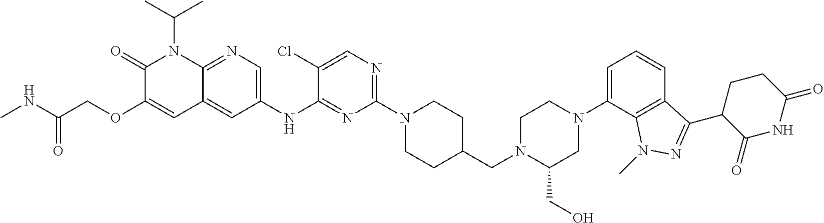 [SMILES: CNC(=O)COc1cc2cc(Nc3nc(N4CCC(CN5CCN(c6cccc7c(C8CCC(=O)NC8=O)nn(C)c67)C[C@@H]5CO)CC4)ncc3Cl)cnc2n(C(C)C)c1=O] |
| 232 | 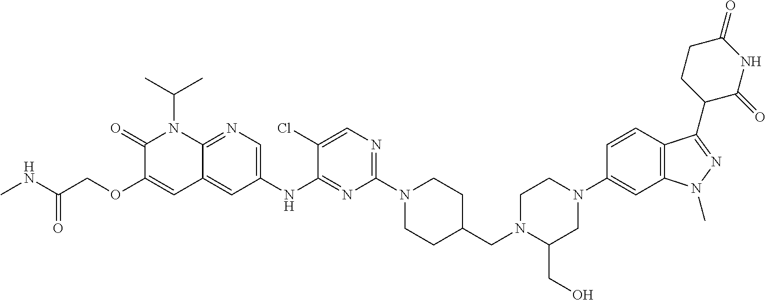 [SMILES: CNC(=O)COc1cc2cc(Nc3nc(N4CCC(CN5CCN(c6ccc7c(C8CCC(=O)NC8=O)nn(C)c7c6)CC5CO)CC4)ncc3Cl)cnc2n(C(C)C)c1=O] |
| 232a | 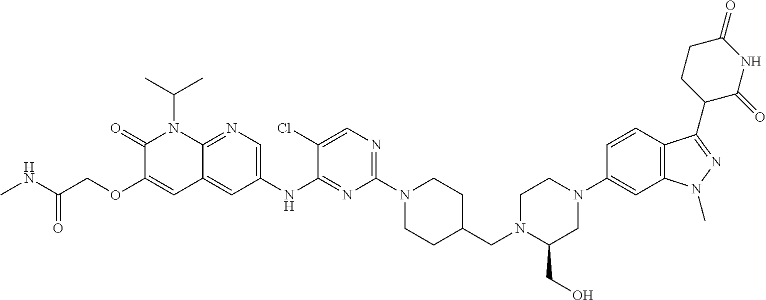 [SMILES: CNC(=O)COc1cc2cc(Nc3nc(N4CCC(CN5CCN(c6ccc7c(C8CCC(=O)NC8=O)nn(C)c7c6)C[C@H]5CO)CC4)ncc3Cl)cnc2n(C(C)C)c1=O] |
| 232b | 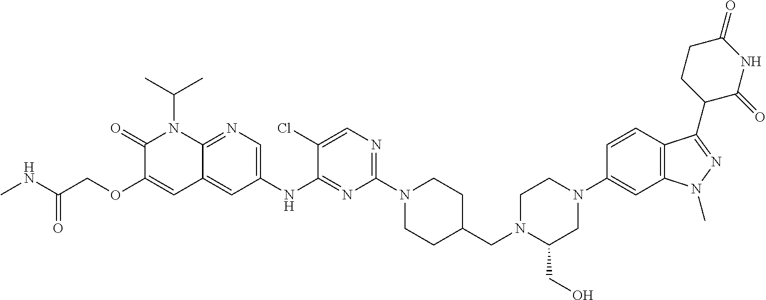 [SMILES: CNC(=O)COc1cc2cc(Nc3nc(N4CCC(CN5CCN(c6ccc7c(C8CCC(=O)NC8=O)nn(C)c7c6)C[C@@H]5CO)CC4)ncc3Cl)cnc2n(C(C)C)c1=O] |
| 233 | 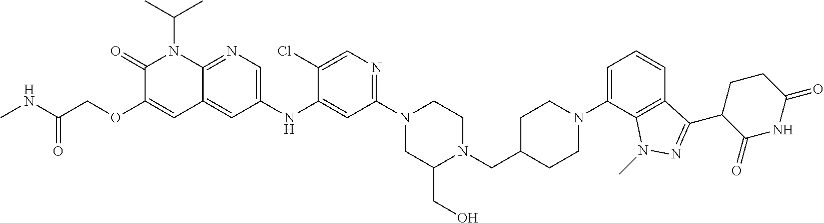 [SMILES: CNC(=O)COc1cc2cc(Nc3cc(N4CCN(CC5CCN(c6cccc7c(C8CCC(=O)NC8=O)nn(C)c67)CC5)C(CO)C4)ncc3Cl)cnc2n(C(C)C)c1=O] |
| 233a | 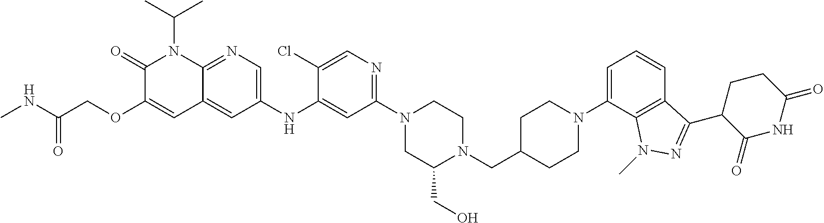 [SMILES: CNC(=O)COc1cc2cc(Nc3cc(N4CCN(CC5CCN(c6cccc7c(C8CCC(=O)NC8=O)nn(C)c67)CC5)[C@H](CO)C4)ncc3Cl)cnc2n(C(C)C)c1=O] |
| 235 | 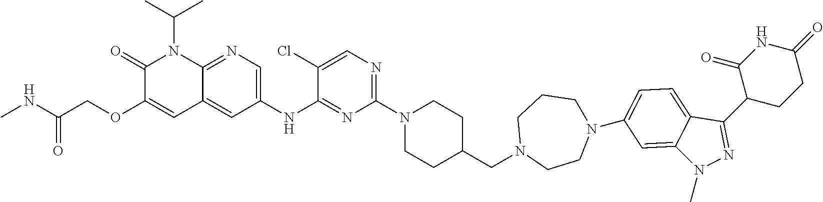 [SMILES: CNC(=O)COc1cc2cc(Nc3nc(N4CCC(CN5CCCN(c6ccc7c(C8CCC(=O)NC8=O)nn(C)c7c6)CC5)CC4)ncc3Cl)cnc2n(C(C)C)c1=O] |
| 236 | 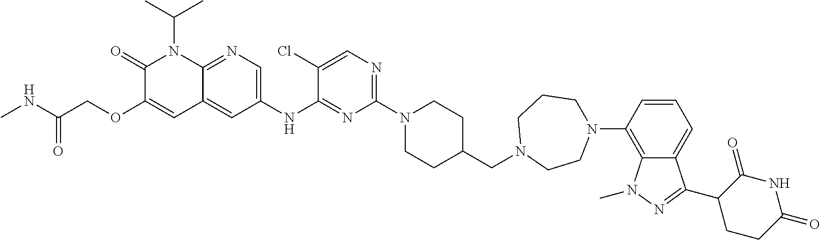 [SMILES: CNC(=O)COc1cc2cc(Nc3nc(N4CCC(CN5CCCN(c6cccc7c(C8CCC(=O)NC8=O)nn(C)c67)CC5)CC4)ncc3Cl)cnc2n(C(C)C)c1=O] |
| 237 | 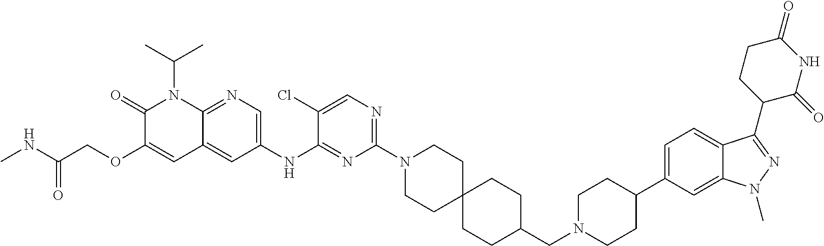 [SMILES: CNC(=O)COc1cc2cc(Nc3nc(N4CCC5(CCC(CN6CCC(c7ccc8c(C9CCC(=O)NC9=O)nn(C)c8c7)CC6)CC5)CC4)ncc3Cl)cnc2n(C(C)C)c1=O] |
| 238 | 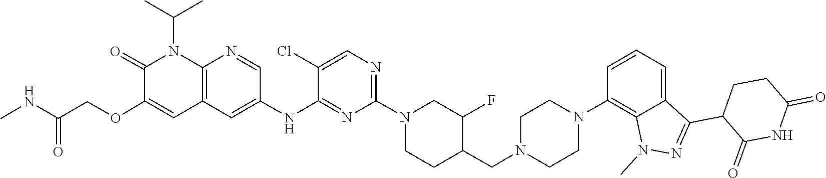 [SMILES: CNC(=O)COc1cc2cc(Nc3nc(N4CCC(CN5CCN(c6cccc7c(C8CCC(=O)NC8=O)nn(C)c67)CC5)C(F)C4)ncc3Cl)cnc2n(C(C)C)c1=O] |
| 238a | 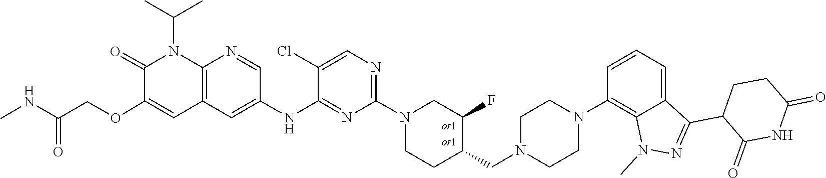 [SMILES: CNC(=O)COc1cc2cc(Nc3nc(N4CC[C@@H](CN5CCN(c6cccc7c(C8CCC(=O)NC8=O)nn(C)c67)CC5)[C@H](F)C4)ncc3Cl)cnc2n(C(C)C)c1=O] |
| 238b | 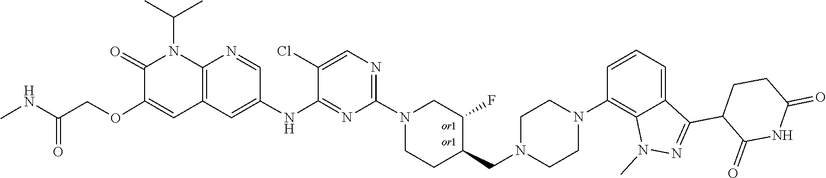 [SMILES: CNC(=O)COc1cc2cc(Nc3nc(N4CC[C@H](CN5CCN(c6cccc7c(C8CCC(=O)NC8=O)nn(C)c67)CC5)[C@@H](F)C4)ncc3Cl)cnc2n(C(C)C)c1=O] |
| 239 | 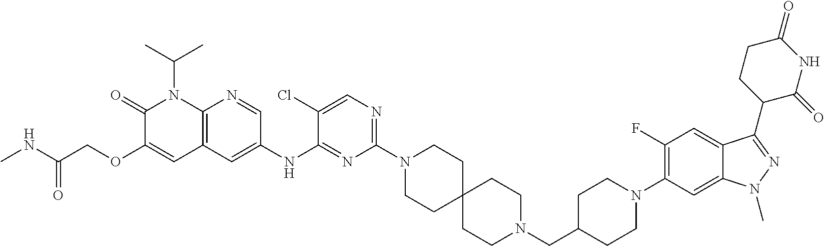 [SMILES: CNC(=O)COc1cc2cc(Nc3nc(N4CCC5(CCN(CC6CCN(c7cc8c(cc7F)c(C7CCC(=O)NC7=O)nn8C)CC6)CC5)CC4)ncc3Cl)cnc2n(C(C)C)c1=O] |
| 240 | 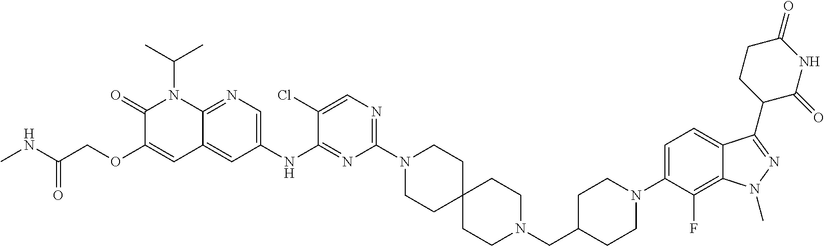 [SMILES: CNC(=O)COc1cc2cc(Nc3nc(N4CCC5(CCN(CC6CCN(c7ccc8c(C9CCC(=O)NC9=O)nn(C)c8c7F)CC6)CC5)CC4)ncc3Cl)cnc2n(C(C)C)c1=O] |
| 241 | 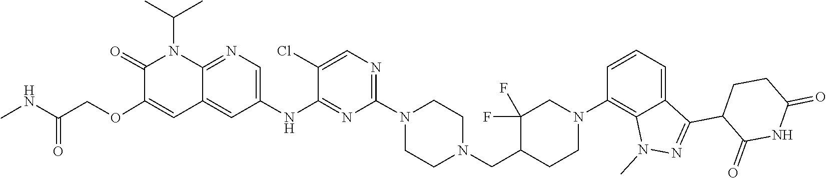 [SMILES: CNC(=O)COc1cc2cc(Nc3nc(N4CCN(CC5CCN(c6cccc7c(C8CCC(=O)NC8=O)nn(C)c67)CC5(F)F)CC4)ncc3Cl)cnc2n(C(C)C)c1=O] |
| 242 | 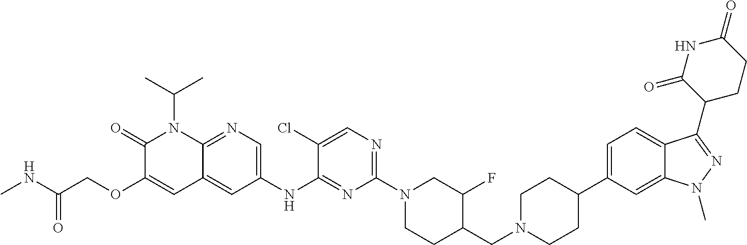 [SMILES: CNC(=O)COc1cc2cc(Nc3nc(N4CCC(CN5CCC(c6ccc7c(C8CCC(=O)NC8=O)nn(C)c7c6)CC5)C(F)C4)ncc3Cl)cnc2n(C(C)C)c1=O] |
| 242a | 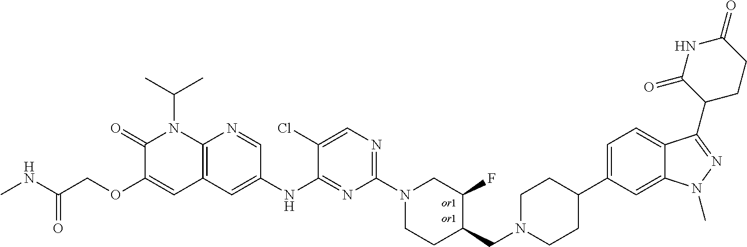 [SMILES: CNC(=O)COc1cc2cc(Nc3nc(N4CC[C@H](CN5CCC(c6ccc7c(C8CCC(=O)NC8=O)nn(C)c7c6)CC5)[C@H](F)C4)ncc3Cl)cnc2n(C(C)C)c1=O] |
| 242b | 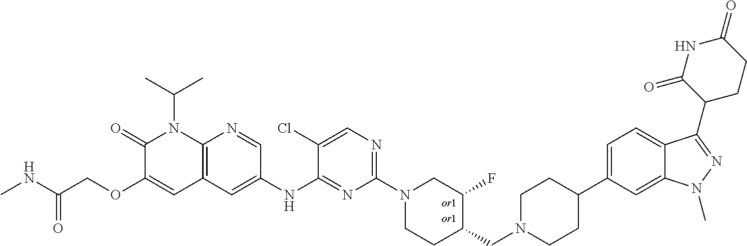 [SMILES: CNC(=O)COc1cc2cc(Nc3nc(N4CC[C@@H](CN5CCC(c6ccc7c(C8CCC(=O)NC8=O)nn(C)c7c6)CC5)[C@@H](F)C4)ncc3Cl)cnc2n(C(C)C)c1=O] |

In certain compounds of Table C1, one or more stereogenic centers are denoted with the “V3000 enhanced stereochemical notation” (see: support.collaborativedrug.com/hc/en-us/articles/360020872171-Advanced-Stereochemistry-Registration-Atropisomers-Mixtures-Unknowns-and-Non-Tetrahedral-Chirality, accessed on Dec. 23, 2022 and Accelrys Chemical Representation Guide, Accelrys Software Inc., 2014, each of which is incorporated by reference herein in its entirety). Using this stereochemical notation, certain stereogenic centers are denoted with “abs”, “&x”, or “orx”, wherein x is an integer (e.g., 1 or 2). For avoidance of doubt, the stereochemical notations in Table C1 have the following meaning:
(1) When a stereogenic center (e.g., a stereogenic carbon) is depicted with “flat” bonds (i.e., none of the chemical bonds at the stereogenic center is depicted with wedges or dashes) in a structural formula, each of said stereogenic centers can independently adopt the (R)- or (S)-configurations. For example, the structure

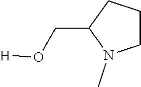

represents (S)-(1-methylpyrrolidin-2-yl)methanol, (R)-(1-methylpyrrolidin-2-yl)methanol, or a mixture thereof. As another non-limiting example, the structure

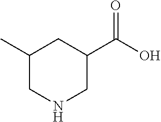

represents: (3S,5S)-5-methylpiperidine-3-carboxylic acid; (3R,5S)-5-methylpiperidine-3-carboxylic acid; (3S,5R)-5-methylpiperidine-3-carboxylic acid; (3R,5R)-5-methylpiperidine-3-carboxylic acid; or a mixture thereof.

When a stereogenic center or a plurality of stereogenic centers is depicted with wedges and dashes, the following notations are used:
(2) When a stereogenic center is denoted with “abs” or when a stereogenic center is not denoted with an enhanced stereochemical notation (e.g., “abs”, “&x”, or “orx”), the stereogenic center has the absolute configuration as depicted by the structural formula. For example, both of the structures

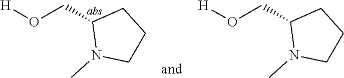

refer to (S)-(1-methylpyrrolidin-2-yl)methanol.
(3) When a stereogenic center is denoted with “orx” in a structural formula, the stereogenic center has been resolved but the configuration at the stereogenic center has not been determined. For example, the structure

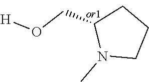

refers to one stereoisomer selected from the group consisting of(S)-(1-methylpyrrolidin-2-yl)methanol and (R)-(1-methylpyrrolidin-2-yl)methanol.
(4) When two or more stereogenic centers are denoted with “orx” in a structural formula, each of these stereogenic centers has been resolved but the configurations at said stereogenic centers have not been determined. Specifically:
   a. For any pair of stereogenic centers denoted with “orx” in a structural formula, when the numerical parts in the notation are different (e.g., two stereogenic centers denoted with “or1” and “or2” respectively), each stereogenic center is independently defined according to (3) (vide supra). For example, the structure

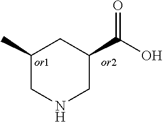

refers to one stereoisomer selected from the group consisting of:

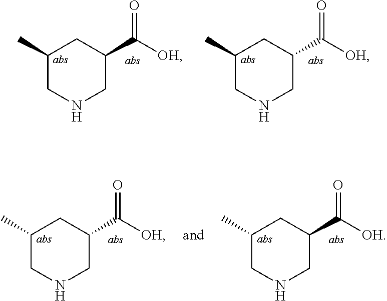

b. For any pair of stereogenic centers denoted with “orx” in a structural formula, when the numerical part in the notation is identical (e.g., two stereogenic centers each denoted with “or1”), the structural formula refers to one stereoisomer having the relative stereochemistry at these stereogenic centers as depicted in the structural formula, but the absolute configurations of these stereogenic centers have not been determined. For example, the structure

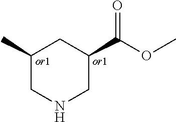

refers to one of the two “syn” stereoisomers:

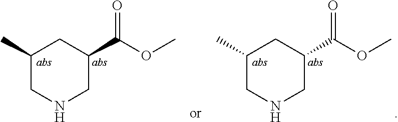

As another example, the structure

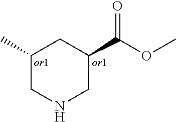

refers to one of the “anti” stereoisomers:

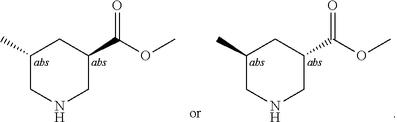

(5) When two or more stereogenic centers are denoted with “&x” in a structural formula, the structural formula refers to a mixture of stereoisomers that differ in the configuration at said stereogenic centers. Specifically:
   a. For any pair of stereogenic centers denoted with “&x” in a structural formula, when the numerical parts in the notation are different (e.g., two stereogenic centers denoted with “&1” and “&2” respectively), the structural formula refers to a mixture of stereoisomers at these two stereogenic centers, wherein the configuration at each stereogenic center can vary independently of one another. For example, the structure

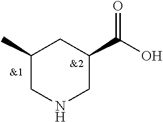

refers to a mixture of four stereoisomers:

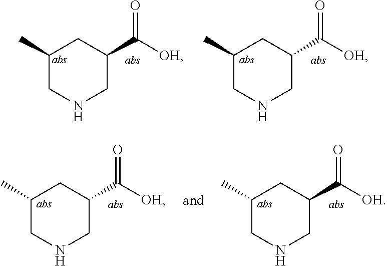

b. For any pair of stereogenic centers denoted with “&x” in a structural formula, when the numerical part in the notation is identical (e.g., two stereogenic centers each denoted with “&l”), the structural formula refers to a mixture of stereoisomers at these two or more stereogenic centers, wherein the relative configurations are as depicted in the structural formula. For example, the structure

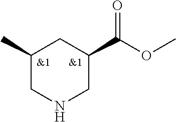

refers to a mixture of “syn” stereoisomers:

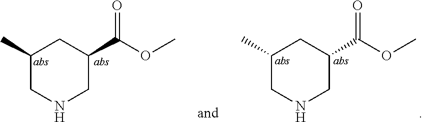

As another example, the structure

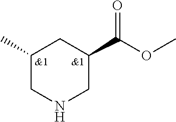

refers to a mixture of “anti” stereoisomers:

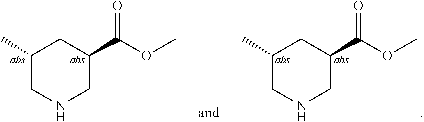

Exemplary compounds of Formula (I-a) include compounds: 101, 103, 104, 105, 105a, 106, 106a, 107, 110, 111, 112, 113, 114, 115, 116, 117, 118, 119, 120, 121, 124, 125, 126, 126a, 127, 127a, 128, 129, 130, 130a, 131, 131a, 132, 133, 134, 135, 136, 136a, 139, 140, 141, 141a, 143, 145, 149, 152, 158, 160, 161, 162, 163, 163a, 167, 168, 168a, 168b, 169, 169a, 169b, 171, 172, 173, 174, 174a, 174b, 175, 175a, 175b, 176, 176a, 176b, 176c, 176d, 177, 177a, 177b, 177c, 177d, 178, 179, 179a, 179b, 187, 188, 188a, 188b, 189, 189a, 190, 191, 192, 192a, 192b, 193, 193a, 195, 195a, 196, 197, 197a, 197b, 200, 203, 204, 205, 209, 210, 210a, 210b, 211, 212, 214, 215, 215a, 215b, 216, 216a, 216b, 216c, 216d, 217, 217a, 217b, 218, 218a, 218b, 219, 220, 223, 224, 225, 228, 228a, 228b, 229, 230, 235, 236, 237, 238, 238a, 238b, 239, 240, 241, 242, 242a, and 242b, as depicted in Table C1, or pharmaceutically acceptable salts thereof.

Exemplary compounds of Formula (I-a-1) include compounds: 101, 103, 104, 105, 105a, 106, 106a, 107, 110, 111, 126, 126a, 127, 127a, 130, 130a, 131, 131a, 132, 133, 134, 135, 139, 140, 141, 141a, 145, 152, 158, 160, 161, 163, 163a, 167, 168, 168a, 168b, 169, 169a, 169b, 171, 172, 176, 176a, 176b, 176c, 176d, 177, 177a, 177b, 177c, 177d, 178, 179, 179a, 179b, 188, 188a, 188b, 189, 189a, 190, 192, 192a, 192b, 193, 193a, 195, 195a, 196, 197, 197a, 197b, 200, 203, 210, 210a, 210b, 212, 214, 215, 215a, 215b, 216, 216a, 216b, 216c, 216d, 217, 217a, 217b, 218, 218a, 218b, 228, 228a, 228b, 235, 236, 238, 238a, 238b, 241, 242, 242a, and 242b, as depicted in Table C1, or pharmaceutically acceptable salts thereof.

Exemplary compounds of Formula (I-a-2) include compounds: 101, 103, 104, 105, 105a, 106, 106a, 107, 126, 126a, 127, 127a, 130, 130a, 131, 131a, 132, 133, 134, 135, 139, 140, 141, 141a, 145, 152, 158, 160, 161, 163, 163a, 167, 168, 168a, 168b, 169, 169a, 169b, 171, 172, 176, 176a, 176b, 176c, 176d, 177, 177a, 177b, 177c, 177d, 178, 179, 179a, 179b, 188, 188a, 188b, 189, 189a, 190, 192, 192a, 192b, 193, 193a, 195, 195a, 196, 197, 197a, 197b, 200, 203, 210, 210a, 210b, 212, 214, 215, 215a, 215b, 217, 217a, 217b, 238, 238a, 238b, 241, 242, 242a, and 242b, as depicted in Table C1, or pharmaceutically acceptable salts thereof.

Exemplary compounds of Formula (I-a-3) include compounds: 112, 113, 118, 119, 120, 121, 124, 128, 136, 136a, 143, 149, 173, 174, 174a, 174b, 175, 175a, 175b, 187, 191, 204, 205, 209, 237, 239, and 240, as depicted in Table C1, or pharmaceutically acceptable salts thereof.

Exemplary compounds of Formula (I-a-4) include compounds: 112, 113, 204, 237, 239, and 240, as depicted in Table C1, or pharmaceutically acceptable salts thereof.

Exemplary compounds of Formula (I-a-5) include compounds: 211, 219, 220, 223, and 224, as depicted in Table C1, or pharmaceutically acceptable salts thereof.

Exemplary compounds of Formula (I-a-6) include compounds: 229 and 230, as depicted in Table C1, or pharmaceutically acceptable salts thereof.

Exemplary compounds of Formula (I-b) include compounds: 122, 122a, 122b, 123, 123a, 123b, 137, 138, 142, 144, 147, 148, 156, 156a, 156b, 156c, 156d, 159, 159a, 159b, 164, 180, 180a, 180b, 180c, 180d, 181, 182, 183, 184, 185, 185a, 185b, 185c, 185d, 198, 199, 202, 207, 213, 221, 221a, 221b, 221c, 221d, and 222, as depicted in Table C1, or pharmaceutically acceptable salts thereof.

Exemplary compounds of Formula (I-b-1) include compounds: 137, 138, 142, 144, 147, 148, 156, 156a, 156b, 156c, 156d, 159, 159a, 159b, 164, 180, 180a, 180b, 180c, 180d, 181, 182, 183, 184, 185, 185a, 185b, 185c, 185d, 198, 199, 202, 207, 213, 221, 221a, 221b, 221c, 221d, and 222, as depicted in Table C1, or pharmaceutically acceptable salts thereof.

Exemplary compounds of Formula (I-b-2) include compounds: 137, 138, 142, 144, 156, 156a, 156b, 156c, 156d, 159, 159a, 159b, 180, 180a, 180b, 180c, 180d, 181, 182, 183, 184, 185, 185a, 185b, 185c, 185d, 198, 221, 221a, 221b, 221c, 221d, and 222, as depicted in Table C1, or pharmaceutically acceptable salts thereof.

In some embodiments, the compounds are selected from the group consisting of the compounds in Table C2, or pharmaceutically acceptable salts thereof.

TABLE C2

| Compound | |
|---|---|
| No. | Compound Name |
| 101 | 2-((6-((5-chloro-2-(4-((1-(3-(2,6-dioxopiperidin-3-yl)-1-methyl-1H-indazol-7-yl)piperidin- |
| | 4-yl)methyl)piperazin-1-yl)pyrimidin-4-yl)amino)-1-methyl-2-oxo-1,2-dihydro-1,8- |
| | naphthyridin-3-yl)oxy)-N-methylacetamide |
| 102 | 2-((6-((5-chloro-2-(4-((1-(2-(2,6-dioxopiperidin-3-yl)-1,3-dioxoisoindolin-4-yl)piperidin-4- |
| | yl)methyl)piperazin-1-yl)pyrimidin-4-yl)amino)-1-methyl-2-oxo-1,2-dihydro-1,8- |
| | naphthyridin-3-yl)oxy)-N-methylacetamide |
| 103 | 2-((6-((5-chloro-2-(4-((1-(3-(2,6-dioxopiperidin-3-yl)-1-methyl-1H-indazol-7-yl)piperidin- |
| | 4-yl)methyl)piperazin-1-yl)pyrimidin-4-yl)amino)-1-isopropyl-2-oxo-1,2-dihydro-1,8- |
| | naphthyridin-3-yl)oxy)-N-methylacetamide |
| 104 | 2-((6-((5-chloro-2-(4-((1-(3-(2,6-dioxopiperidin-3-yl)-1-methyl-1H-indazol-6-yl)piperidin- |
| | 4-yl)methyl)piperazin-1-yl)pyrimidin-4-yl)amino)-1-isopropyl-2-oxo-1,2-dihydro-1,8- |
| | naphthyridin-3-yl)oxy)-N-methylacetamide |
| 105 | 2-[[6-[[5-chloro-2-[4-[[1-[3-(2,6-dioxo-3-piperidyl)-1-methyl-indazol-7-yl]-4- |
| | piperidyl]methyl]-3-methyl-piperazin-1-yl]pyrimidin-4-yl]amino]-1-isopropyl-2-oxo-1,8- |
| | naphthyridin-3-yl]oxy]-N-methyl-acetamide |
| 105a | 2-((6-((5-chloro-2-((3S)-4-((1-(3-(2,6-dioxopiperidin-3-yl)-1-methyl-1H-indazol-7- |
| | yl)piperidin-4-yl)methyl)-3-methylpiperazin-1-yl)pyrimidin-4-yl)amino)-1-isopropyl-2-oxo- |
| | 1,2-dihydro-1,8-naphthyridin-3-yl)oxy)-N-methylacetamide |
| 106 | 2-[[6-[[5-chloro-2-[4-[[1-[3-(2,6-dioxo-3-piperidyl)-1-methyl-indazol-6-yl]-4- |
| | piperidyl]methyl]-3-methyl-piperazin-1-yl]pyrimidin-4-yl]amino]-1-isopropyl-2-oxo-1,8- |
| | naphthyridin-3-yl]oxy]-N-methyl-acetamide |
| 106a | 2-((6-((5-chloro-2-((3S)-4-((1-(3-(2,6-dioxopiperidin-3-yl)-1-methyl-1H-indazol-6- |
| | yl)piperidin-4-yl)methyl)-3-methylpiperazin-1-yl)pyrimidin-4-yl)amino)-1-isopropyl-2-oxo- |
| | 1,2-dihydro-1,8-naphthyridin-3-yl)oxy)-N-methylacetamide |
| 107 | 2-((6-((5-chloro-2-(4-((1-(3-(2,6-dioxopiperidin-3-yl)-1-methyl-1H-indazol-6-yl)piperidin- |
| | 4-yl)methyl)piperazin-1-yl)pyrimidin-4-yl)amino)-1-methyl-2-oxo-1,2-dihydro-1,8- |
| | naphthyridin-3-yl)oxy)-N-methylacetamide |
| 108 | 2-((6-((5-chloro-2-(4-((1-(3-(2,4-dioxotetrahydropyrimidin-1(2H)-yl)-1-methyl-1H-indazol- |
| | 7-yl)piperidin-4-yl)methyl)piperazin-1-yl)pyrimidin-4-yl)amino)-1-methyl-2-oxo-1,2- |
| | dihydro-1,8-naphthyridin-3-yl)oxy)-N-methylacetamide |
| 109 | 2-((6-((5-chloro-2-(4-((1-(3-(2,4-dioxotetrahydropyrimidin-1(2H)-yl)-1-methyl-1H-indazol- |
| | 7-yl) piperidin-4-yl) methyl) piperazin-1-yl) pyrimidin-4-yl) amino)-1-isopropyl-2-oxo-1,2- |
| | dihydro-1,8-naphthyridin-3-yl) oxy)-N-methylacetamide |
| 110 | 2-((6-((5-chloro-2-(4-((1-(3-(2,6-dioxopiperidin-3-yl)-1-methyl-1H-indazol-7-yl)piperidin- |
| | 4-yl)methyl)-1,4-diazepan-1-yl)pyrimidin-4-yl)amino)-1-methyl-2-oxo-1,2-dihydro-1,8- |
| | naphthyridin-3-yl)oxy)-N-methylacetamide |
| 111 | 2-((6-((5-chloro-2-(4-((1-(3-(2,6-dioxopiperidin-3-yl)-1-methyl-1H-indazol-6-yl)piperidin- |
| | 4-yl)methyl)-1,4-diazepan-1-yl)pyrimidin-4-yl)amino)-1-methyl-2-oxo-1,2-dihydro-1,8- |
| | naphthyridin-3-yl)oxy)-N-methylacetamide |
| 112 | 2-((6-((5-chloro-2-(9-((1-(3-(2,6-dioxopiperidin-3-yl)-1-methyl-1H-indazol-6-yl)piperidin- |
| | 4-yl)methyl)-3,9-diazaspiro[5.5]undecan-3-yl)pyrimidin-4-yl)amino)-1-methyl-2-oxo-1,2- |
| | dihydro-1,8-naphthyridin-3-yl)oxy)-N-methylacetamide |
| 113 | 2-((6-((5-chloro-2-(9-((1-(3-(2,6-dioxopiperidin-3-yl)-1-methyl-1H-indazol-7-yl)piperidin- |
| | 4-yl)methyl)-3,9-diazaspiro[5.5]undecan-3-yl)pyrimidin-4-yl)amino)-1-methyl-2-oxo-1,2- |
| | dihydro-1,8-naphthyridin-3-yl)oxy)-N-methylacetamide |
| 114 | 2-((6-((5-chloro-2-(4-(2-(1-(3-(2,6-dioxopiperidin-3-yl)-1-methyl-1H-indazol-6- |
| | yl)piperidin-4-yl)ethyl)piperazin-1-yl)pyrimidin-4-yl)amino)-1-methyl-2-oxo-1,2-dihydro- |
| | 1,8-naphthyridin-3-yl)oxy)-N-methylacetamide |
| 115 | 2-((6-((5-chloro-2-(4-(2-(1-(3-(2,6-dioxopiperidin-3-yl)-1-methyl-1H-indazol-7- |
| | yl)piperidin-4-yl)ethyl)piperazin-1-yl)pyrimidin-4-yl)amino)-1-methyl-2-oxo-1,2-dihydro- |
| | 1,8-naphthyridin-3-yl)oxy)-N-methylacetamide |
| 116 | 2-[[6-[[5-chloro-2-[9-[[1-[3-(2,6-dioxo-3-piperidyl)-1-methyl-indazol-6-yl]-4- |
| | piperidyl]methyl]-3-oxa-7,9-diazabicyclo[3.3.1]nonan-7-yl]pyrimidin-4-yl]amino]-1- |
| | methyl-2-oxo-1,8-naphthyridin-3-yl]oxy]-N-methyl-acetamide |
| 117 | 2-((6-((5-chloro-2-(9-((1-(3-(2,6-dioxopiperidin-3-yl)-1-methyl-1H-indazol-7-yl)piperidin- |
| | 4-yl)methyl)-3-oxa-7,9-diazabicyclo[3.3.1]nonan-7-yl)pyrimidin-4-yl)amino)-1-methyl-2- |
| | oxo-1,2-dihydro-1,8-naphthyridin-3-yl)oxy)-N-methylacetamide |
| 118 | 2-((6-((5-chloro-2-(6-((1-(3-(2,6-dioxopiperidin-3-yl)-1-methyl-1H-indazol-6-yl)piperidin- |
| | 4-yl)methyl)-2,6-diazaspiro[3.3]heptan-2-yl)pyrimidin-4-yl)amino)-1-methyl-2-oxo-1,2- |
| | dihydro-1,8-naphthyridin-3-yl)oxy)-N-methylacetamide |
| 119 | 2-((6-((5-chloro-2-(6-((1-(3-(2,6-dioxopiperidin-3-yl)-1-methyl-1H-indazol-7-yl)piperidin- |
| | 4-yl)methyl)-2,6-diazaspiro[3.3]heptan-2-yl)pyrimidin-4-yl)amino)-1-methyl-2-oxo-1,2- |
| | dihydro-1,8-naphthyridin-3-yl)oxy)-N-methylacetamide |
| 120 | 2-((6-((5-chloro-2-(2-((1-(3-(2,6-dioxopiperidin-3-yl)-1-methyl-1H-indazol-7-yl)piperidin- |
| | 4-yl)methyl)-2,7-diazaspiro[3.5]nonan-7-yl)pyrimidin-4-yl)amino)-1-methyl-2-oxo-1,2- |
| | dihydro-1,8-naphthyridin-3-yl)oxy)-N-methylacetamide |
| 121 | 2-((6-((5-chloro-2-(2-((1-(3-(2,6-dioxopiperidin-3-yl)-1-methyl-1H-indazol-6-yl)piperidin- |
| | 4-yl)methyl)-2,7-diazaspiro[3.5]nonan-7-yl)pyrimidin-4-yl)amino)-1-methyl-2-oxo-1,2- |
| | dihydro-1,8-naphthyridin-3-yl)oxy)-N-methylacetamide |
| 122 | 2-[[6-[[5-chloro-2-[3-[[[3-(2,6-dioxo-3-piperidyl)-1-methyl-indazol-7- |
| | yl]amino]methyl]pyrrolidin-1-yl]pyrimidin-4-yl]amino]-1-methyl-2-oxo-1,8-naphthyridin- |
| | 3-yl]oxy]-N-methyl-acetamide |
| 122a | 2-((6-((5-chloro-2-((3R)-3-(((3-(2,6-dioxopiperidin-3-yl)-1-methyl-1H-indazol-7- |
| | yl)amino)methyl)pyrrolidin-1-yl)pyrimidin-4-yl)amino)-1-methyl-2-oxo-1,2-dihydro-1,8- |
| | naphthyridin-3-yl)oxy)-N-methylacetamide |
| 122b | 2-((6-((5-chloro-2-((3S)-3-(((3-(2,6-dioxopiperidin-3-yl)-1-methyl-1H-indazol-7- |
| | yl)amino)methyl)pyrrolidin-1-yl)pyrimidin-4-yl)amino)-1-methyl-2-oxo-1,2-dihydro-1,8- |
| | naphthyridin-3-yl)oxy)-N-methylacetamide |
| 123 | 2-[[6-[[5-chloro-2-[3-[[[3-(2,6-dioxo-3-piperidyl)-1-methyl-indazol-6- |
| | yl]amino]methyl]pyrrolidin-1-yl]pyrimidin-4-yl]amino]-1-methyl-2-oxo-1,8-naphthyridin- |
| | 3-yl]oxy]-N-methyl-acetamide |
| 123a | 2-((6-((5-chloro-2-((3R)-3-(((3-(2,6-dioxopiperidin-3-yl)-1-methyl-1H-indazol-6- |
| | yl)amino)methyl)pyrrolidin-1-yl)pyrimidin-4-yl)amino)-1-methyl-2-oxo-1,2-dihydro-1,8- |
| | naphthyridin-3-yl)oxy)-N-methylacetamide |
| 123b | 2-((6-((5-chloro-2-((3S)-3-(((3-(2,6-dioxopiperidin-3-yl)-1-methyl-1H-indazol-6- |
| | yl)amino)methyl)pyrrolidin-1-yl)pyrimidin-4-yl)amino)-1-methyl-2-oxo-1,2-dihydro-1,8- |
| | naphthyridin-3-yl)oxy)-N-methylacetamide |
| 124 | 2-[[6-[[5-chloro-2-[4-[[2-[3-(2,6-dioxo-3-piperidyl)-1-methyl-indazol-6-yl]-2,7- |
| | diazaspiro[3.5]nonan-7-yl]methyl]-1-piperidyl]pyrimidin-4-yl]amino]-1-isopropyl-2-oxo- |
| | 1,8-naphthyridin-3-yl]oxy]-N-methyl-acetamide |
| 125 | 2-[[6-[[5-chloro-2-[3-[[2-[3-(2,6-dioxo-3-piperidyl)-1-methyl-indazol-6-yl]-2- |
| | azaspiro[3.5]nonan-7-yl]methyl]-3,6-diazabicyclo[3.1.1]heptan-6-yl]pyrimidin-4-yl]amino]- |
| | 1-methyl-2-oxo-1,8-naphthyridin-3-yl]oxy]-N-methyl-acetamide |
| 126 | 2-[[6-[[5-chloro-2-[4-[[1-[3-(2,6-dioxo-3-piperidyl)-1-methyl-indazol-6-yl]-3,5-dimethyl-4- |
| | piperidyl]methyl]piperazin-1-yl]pyrimidin-4-yl]amino]-1-isopropyl-2-oxo-1,8-naphthyridin- |
| | 3-yl]oxy]-N-methyl-acetamide |
| 126a | 2-[[6-[[5-chloro-2-[4-[[(3S,5R)-1-[3-(2,6-dioxo-3-piperidyl)-1-methyl-indazol-6-yl]-3,5- |
| | dimethyl-4-piperidyl]methyl]piperazin-1-yl]pyrimidin-4-yl]amino]-1-isopropyl-2-oxo-1,8- |
| | naphthyridin-3-yl]oxy]-N-methyl-acetamide |
| 127 | 2-[[6-[[5-chloro-2-[4-[[1-[3-(2,6-dioxo-3-piperidyl)-1-methyl-indazol-7-yl]-2-methyl-4- |
| | piperidyl]methyl]piperazin-1-yl]pyrimidin-4-yl]amino]-1-isopropyl-2-oxo-1,8-naphthyridin- |
| | 3-yl]oxy]-N-methyl-acetamide |
| 127a | 2-[[6-[[5-chloro-2-[4-[[(2S)-1-[3-(2,6-dioxo-3-piperidyl)-1-methyl-indazol-7-yl]-2-methyl- |
| | 4-piperidyl]methyl]piperazin-1-yl]pyrimidin-4-yl]amino]-1-isopropyl-2-oxo-1,8- |
| | naphthyridin-3-yl]oxy]-N-methyl-acetamide |
| 128 | 2-[[6-[[2-[4-[[2-[3-(2,6-dioxo-3-piperidyl)-1-methyl-indazol-6-yl]-2,7- |
| | diazaspiro[3.5]nonan-7-yl]methyl]-1-piperidyl]-5-fluoro-pyrimidin-4-yl]amino]-1- |
| | isopropyl-2-oxo-1,8-naphthyridin-3-yl]oxy]-N-methyl-acetamide |
| 129 | 2-[[6-[[2-[3-[[2-[3-(2,6-dioxo-3-piperidyl)-1-methyl-indazol-6-yl]-2-azaspiro[3.5]nonan-7- |
| | yl]methyl]-3,6-diazabicyclo[3.1.1]heptan-6-yl]-5-fluoro-pyrimidin-4-yl]amino]-1-methyl-2- |
| | oxo-1,8-naphthyridin-3-yl]oxy]-N-methyl-acetamide |
| 130 | 2-[[6-[[2-[4-[[1-[3-(2,6-dioxo-3-piperidyl)-1-methyl-indazol-6-yl]-3,5-dimethyl-4- |
| | piperidyl]methyl]piperazin-1-yl]-5-fluoro-pyrimidin-4-yl]amino]-1-isopropyl-2-oxo-1,8- |
| | naphthyridin-3-yl]oxy]-N-methyl-acetamide |
| 130a | 2-[[6-[[2-[4-[[(3S,5R)-1-[3-(2,6-dioxo-3-piperidyl)-1-methyl-indazol-6-yl]-3,5-dimethyl-4- |
| | piperidyl]methyl]piperazin-1-yl]-5-fluoro-pyrimidin-4-yl]amino]-1-isopropyl-2-oxo-1,8- |
| | naphthyridin-3-yl]oxy]-N-methyl-acetamide |
| 131 | 2-[[6-[[2-[4-[[1-[3-(2,6-dioxo-3-piperidyl)-1-methyl-indazol-7-yl]-2-methyl-4- |
| | piperidyl]methyl]piperazin-1-yl]-5-fluoro-pyrimidin-4-yl]amino]-1-isopropyl-2-oxo-1,8- |
| | naphthyridin-3-yl]oxy]-N-methyl-acetamide |
| 131a | 2-[[6-[[2-[4-[[(2S)-1-[3-(2,6-dioxo-3-piperidyl)-1-methyl-indazol-7-yl]-2-methyl-4- |
| | piperidyl]methyl]piperazin-1-yl]-5-fluoro-pyrimidin-4-yl]amino]-1-isopropyl-2-oxo-1,8- |
| | naphthyridin-3-yl]oxy]-N-methyl-acetamide |
| 132 | 2-((6-((5-chloro-2-(4-((4-(3-(2,6-dioxopiperidin-3-yl)-1-methyl-1H-indazol-7-yl)piperazin- |
| | 1-yl)methyl)piperidin-1-yl)pyrimidin-4-yl)amino)-1-methyl-2-oxo-1,2-dihydro-1,8- |
| | naphthyridin-3-yl)oxy)-N-methylacetamide |
| 133 | 2-((6-((5-chloro-2-(4-((4-(3-(2,6-dioxopiperidin-3-yl)-1-methyl-1H-indazol-6-yl)piperazin- |
| | 1-yl)methyl)piperidin-1-yl)pyrimidin-4-yl)amino)-1-methyl-2-oxo-1,2-dihydro-1,8- |
| | naphthyridin-3-yl)oxy)-N-methylacetamide |
| 134 | 2-((6-((2-(4-((1-(3-(2,6-dioxopiperidin-3-yl)-1-methyl-1H-indazol-6-yl)piperidin-4- |
| | yl)methyl)piperazin-1-yl)-5-fluoropyrimidin-4-yl)amino)-1-methyl-2-oxo-1,2-dihydro-1,8- |
| | naphthyridin-3-yl)oxy)-N-methylacetamide |
| 135 | 2-((6-((2-(4-((1-(3-(2,6-dioxopiperidin-3-yl)-1-methyl-1H-indazol-7-yl)piperidin-4- |
| | yl)methyl)piperazin-1-yl)-5-fluoropyrimidin-4-yl)amino)-1-methyl-2-oxo-1,2-dihydro-1,8- |
| | naphthyridin-3-yl)oxy)-N-methylacetamide |
| 136 | 2-[[6-[[5-chloro-2-[6-[[4-[3-(2,6-dioxo-3-piperidyl)-1-methyl-indazol-7-yl]piperazin-1- |
| | yl]methyl]-3-azabicyclo[3.1.0]hexan-3-yl]pyrimidin-4-yl]amino]-1-methyl-2-oxo-1,8- |
| | naphthyridin-3-yl]oxy]-N-methyl-acetamide |
| 136a | 2-((6-((5-chloro-2-((1R,5S,6s)-6-((4-(3-(2,6-dioxopiperidin-3-yl)-1-methyl-1H-indazol-7- |
| | yl)piperazin-1-yl)methyl)-3-azabicyclo[3.1.0]hexan-3-yl)pyrimidin-4-yl)amino)-1-methyl-2- |
| | oxo-1,2-dihydro-1,8-naphthyridin-3-yl)oxy)-N-methylacetamide |
| 137 | 2-((6-((5-chloro-2-(7-((3-(2,6-dioxopiperidin-3-yl)-1-methyl-1H-indazol-7-yl)amino)-2- |
| | azaspiro[3.5]nonan-2-yl)pyrimidin-4-yl)amino)-1-methyl-2-oxo-1,2-dihydro-1,8- |
| | naphthyridin-3-yl)oxy)-N-methylacetamide |
| 138 | 2-((6-((5-chloro-2-(7-((3-(2,6-dioxopiperidin-3-yl)-1-methyl-1H-indazol-6-yl)amino)-2- |
| | azaspiro[3.5]nonan-2-yl)pyrimidin-4-yl)amino)-1-methyl-2-oxo-1,2-dihydro-1,8- |
| | naphthyridin-3-yl)oxy)-N-methylacetamide |
| 139 | 2-((6-((5-chloro-2-(4-((1-(3-(2,6-dioxopiperidin-3-yl)-1-methyl-1H-indazol-7-yl)piperidin- |
| | 4-yl)methyl)piperidin-1-yl)pyrimidin-4-yl)amino)-1-methyl-2-oxo-1,2-dihydro-1,8- |
| | naphthyridin-3-yl)oxy)-N-methylacetamide |
| 140 | 2-((6-((5-chloro-2-(4-((1-(3-(2,6-dioxopiperidin-3-yl)-1-methyl-1H-indazol-6-yl)piperidin- |
| | 4-yl)methyl)piperidin-1-yl)pyrimidin-4-yl)amino)-1-methyl-2-oxo-1,2-dihydro-1,8- |
| | naphthyridin-3-yl)oxy)-N-methylacetamide |
| 141 | 2-[[6-[[5-chloro-2-[4-[[1-[3-(2,6-dioxo-3-piperidyl)-1-methyl-indazol-7-yl]-4- |
| | piperidyl]methyl]-3-(trifluoromethyl)piperazin-1-yl]pyrimidin-4-yl]amino]-1-methyl-2-oxo- |
| | 1,8-naphthyridin-3-yl]oxy]-N-methyl-acetamide |
| 141a | 2-((6-((5-chloro-2-((3R)-4-((1-(3-(2,6-dioxopiperidin-3-yl)-1-methyl-1H-indazol-7- |
| | yl)piperidin-4-yl)methyl)-3-(trifluoromethyl)piperazin-1-yl)pyrimidin-4-yl)amino)-1- |
| | methyl-2-oxo-1,2-dihydro-1,8-naphthyridin-3-yl)oxy)-N-methylacetamide |
| 142 | 2-((6-((5-chloro-2-(7-((3-(2,6-dioxopiperidin-3-yl)-1-methyl-1H-indazol-7-yl)oxy)-2- |
| | azaspiro[3.5]nonan-2-yl)pyrimidin-4-yl)amino)-1-methyl-2-oxo-1,2-dihydro-1,8- |
| | naphthyridin-3-yl)oxy)-N-methylacetamide |
| 143 | 2-((6-((5-chloro-2-(4-(((1R,3s,5S)-3-(3-(2,6-dioxopiperidin-3-yl)-1-methyl-1H-indazol-7- |
| | yl)-8-azabicyclo[3.2.1]octan-8-yl)methyl)piperidin-1-yl)pyrimidin-4-yl)amino)-1-methyl-2- |
| | oxo-1,2-dihydro-1,8-naphthyridin-3-yl)oxy)-N-methylacetamide |
| 144 | 2-((6-((5-chloro-2-(7-((3-(2,6-dioxopiperidin-3-yl)-1-methyl-1H-indazol-6-yl)oxy)-2- |
| | azaspiro[3.5]nonan-2-yl)pyrimidin-4-yl)amino)-1-methyl-2-oxo-1,2-dihydro-1,8- |
| | naphthyridin-3-yl)oxy)-N-methylacetamide |
| 145 | 2-((6-((5-chloro-2-(4-((1-(3-(2,6-dioxopiperidin-3-yl)-1-methyl-1H-indazol-7-yl)piperidin- |
| | 4-yl)methyl)piperazin-1-yl)pyrimidin-4-yl)amino)-1-ethyl-2-oxo-1,2-dihydro-1,8- |
| | naphthyridin-3-yl)oxy)-N-methylacetamide |
| 146 | 2-[[6-[[5-chloro-2-[4-[[1-[3-(2,6-dioxo-3-piperidyl)-1-methyl-indazol-7-yl]-4- |
| | piperidyl]methyl]-3-(hydroxymethyl)piperazin-1-yl]pyrimidin-4-yl]amino]-1-isopropyl-2- |
| | oxo-1,8-naphthyridin-3-yl]oxy]-N-methyl-acetamide |
| 146a | 2-((6-((5-chloro-2-((3S)-4-((1-(3-(2,6-dioxopiperidin-3-yl)-1-methyl-1H-indazol-7- |
| | yl)piperidin-4-yl)methyl)-3-(hydroxymethyl)piperazin-1-yl)pyrimidin-4-yl)amino)-1- |
| | isopropyl-2-oxo-1,2-dihydro-1,8-naphthyridin-3-yl)oxy)-N-methylacetamide |
| 146b | 2-((6-((5-chloro-2-((3R)-4-((1-(3-(2,6-dioxopiperidin-3-yl)-1-methyl-1H-indazol-7- |
| | yl)piperidin-4-yl)methyl)-3-(hydroxymethyl)piperazin-1-yl)pyrimidin-4-yl)amino)-1- |
| | isopropyl-2-oxo-1,2-dihydro-1,8-naphthyridin-3-yl)oxy)-N-methylacetamide |
| 147 | 2-((6-((5-chloro-2-(4-((3-(2,6-dioxopiperidin-3-yl)-1-methyl-1H-indazol-6-yl)oxy)piperidin- |
| | 1-yl)pyrimidin-4-yl)amino)-1-methyl-2-oxo-1,2-dihydro-1,8-naphthyridin-3-yl)oxy)-N- |
| | methylacetamide |
| 148 | 2-((6-((5-chloro-2-(4-((3-(2,6-dioxopiperidin-3-yl)-1-methyl-1H-indazol-7-yl)oxy)piperidin- |
| | 1-yl)pyrimidin-4-yl)amino)-1-methyl-2-oxo-1,2-dihydro-1,8-naphthyridin-3-yl)oxy)-N- |
| | methylacetamide |
| 149 | 2-((6-((5-chloro-2-(4-((2-(3-(2,6-dioxopiperidin-3-yl)-1-methyl-1H-indazol-7-yl)-2- |
| | azaspiro[3.5]nonan-7-yl)methyl)piperazin-1-yl)pyrimidin-4-yl)amino)-1-methyl-2-oxo-1,2- |
| | dihydro-1,8-naphthyridin-3-yl)oxy)-N-methylacetamide |
| 150 | 2-((6-((5-chloro-2-(4-((1-(3-(2,6-dioxopiperidin-3-yl)-1-methyl-1H-indazol-7-yl)piperidin- |
| | 4-yl)methyl)piperazin-1-yl)pyrimidin-4-yl)amino)-1-cyclopropyl-2-oxo-1,2-dihydro-1,8- |
| | naphthyridin-3-yl)oxy)-N-methylacetamide |
| 151 | 2-[[6-[[5-chloro-2-[4-[[1-[3-(2,6-dioxo-3-piperidyl)-1-methyl-indazol-6-yl]-4- |
| | piperidyl]methyl]-2-(hydroxymethyl)piperazin-1-yl]pyrimidin-4-yl]amino]-1-isopropyl-2- |
| | oxo-1,8-naphthyridin-3-yl]oxy]-N-methyl-acetamide |
| 151a | 2-((6-((5-chloro-2-((2R)-4-((1-(3-(2,6-dioxopiperidin-3-yl)-1-methyl-1H-indazol-6- |
| | yl)piperidin-4-yl)methyl)-2-(hydroxymethyl)piperazin-1-yl)pyrimidin-4-yl)amino)-1- |
| | isopropyl-2-oxo-1,2-dihydro-1,8-naphthyridin-3-yl)oxy)-N-methylacetamide |
| 151b | 2-((6-((5-chloro-2-((2S)-4-((1-(3-(2,6-dioxopiperidin-3-yl)-1-methyl-1H-indazol-6- |
| | yl)piperidin-4-yl)methyl)-2-(hydroxymethyl)piperazin-1-yl)pyrimidin-4-yl)amino)-1- |
| | isopropyl-2-oxo-1,2-dihydro-1,8-naphthyridin-3-yl)oxy)-N-methylacetamide |
| 152 | 2-((6-((5-chloro-2-(4-((4-(3-(2,6-dioxopiperidin-3-yl)-1-methyl-1H-indazol-7-yl)piperazin- |
| | 1-yl)methyl)piperidin-1-yl)pyridin-4-yl)amino)-1-isopropyl-2-oxo-1,2-dihydro-1,8- |
| | naphthyridin-3-yl)oxy)-N-methylacetamide |
| 153 | 2-[[6-[[5-chloro-2-[4-[[1-[3-(2,6-dioxo-3-piperidyl)-1-methyl-indazol-6-yl]-4- |
| | piperidyl]methyl]-3-(hydroxymethyl)piperazin-1-yl]pyrimidin-4-yl]amino]-1-isopropyl-2- |
| | oxo-1,8-naphthyridin-3-yl]oxy]-N-methyl-acetamide |
| 153a | 2-((6-((5-chloro-2-((3R)-4-((1-(3-(2,6-dioxopiperidin-3-yl)-1-methyl-1H-indazol-6- |
| | yl)piperidin-4-yl)methyl)-3-(hydroxymethyl)piperazin-1-yl)pyrimidin-4-yl)amino)-1- |
| | isopropyl-2-oxo-1,2-dihydro-1,8-naphthyridin-3-yl)oxy)-N-methylacetamide |
| 153b | 2-((6-((5-chloro-2-((3S)-4-((1-(3-(2,6-dioxopiperidin-3-yl)-1-methyl-1H-indazol-6- |
| | yl)piperidin-4-yl)methyl)-3-(hydroxymethyl)piperazin-1-yl)pyrimidin-4-yl)amino)-1- |
| | isopropyl-2-oxo-1,2-dihydro-1,8-naphthyridin-3-yl)oxy)-N-methylacetamide |
| 154 | 2-((6-((5-chloro-2-(4-((1-(3-(2,6-dioxopiperidin-3-yl)-1-methyl-1H-indazol-7-yl)piperidin- |
| | 4-yl)methyl)piperazin-1-yl)pyrimidin-4-yl)amino)-1-(2-methoxyethyl)-2-oxo-1,2-dihydro- |
| | 1,8-naphthyridin-3-yl)oxy)-N-methylacetamide |
| 155 | 2-[[6-[[5-chloro-2-[4-[[1-[3-(2,6-dioxo-3-piperidyl)-1-methyl-indazol-7-yl]-4- |
| | piperidyl]methyl]-2-(hydroxymethyl)piperazin-1-yl]pyrimidin-4-yl]amino]-1-isopropyl-2- |
| | oxo-1,8-naphthyridin-3-yl]oxy]-N-methyl-acetamide |
| 155a | 2-((6-((5-chloro-2-((2S)-4-((1-(3-(2,6-dioxopiperidin-3-yl)-1-methyl-1H-indazol-7- |
| | yl)piperidin-4-yl)methyl)-2-(hydroxymethyl)piperazin-1-yl)pyrimidin-4-yl)amino)-1- |
| | isopropyl-2-oxo-1,2-dihydro-1,8-naphthyridin-3-yl)oxy)-N-methylacetamide |
| 155b | 2-((6-((5-chloro-2-((2R)-4-((1-(3-(2,6-dioxopiperidin-3-yl)-1-methyl-1H-indazol-7- |
| | yl)piperidin-4-yl)methyl)-2-(hydroxymethyl)piperazin-1-yl)pyrimidin-4-yl)amino)-1- |
| | isopropyl-2-oxo-1,2-dihydro-1,8-naphthyridin-3-yl)oxy)-N-methylacetamide |
| 156 | 2-[[6-[[5-chloro-2-[7-[[3-(2,6-dioxo-3-piperidyl)-1-methyl-indazol-6-yl]amino]-6-methyl-2- |
| | azaspiro[3.5]nonan-2-yl]pyrimidin-4-yl]amino]-1-methyl-2-oxo-1,8-naphthyridin-3-yl]oxy]- |
| | N-methyl-acetamide |
| 156a | rel-2-((6-((5-chloro-2-((6S,7S)-7-((3-(2,6-dioxopiperidin-3-yl)-1-methyl-1H-indazol-6- |
| | yl)amino)-6-methyl-2-azaspiro[3.5]nonan-2-yl)pyrimidin-4-yl)amino)-1-methyl-2-oxo-1,2- |
| | dihydro-1,8-naphthyridin-3-yl)oxy)-N-methylacetamide |
| 156b | rel-2-((6-((5-chloro-2-((6R,7S)-7-((3-(2,6-dioxopiperidin-3-yl)-1-methyl-1H-indazol-6- |
| | yl)amino)-6-methyl-2-azaspiro[3.5]nonan-2-yl)pyrimidin-4-yl)amino)-1-methyl-2-oxo-1,2- |
| | dihydro-1,8-naphthyridin-3-yl)oxy)-N-methylacetamide |
| 156c | rel-2-((6-((5-chloro-2-((6S,7R)-7-((3-(2,6-dioxopiperidin-3-yl)-1-methyl-1H-indazol-6- |
| | yl)amino)-6-methyl-2-azaspiro[3.5]nonan-2-yl)pyrimidin-4-yl)amino)-1-methyl-2-oxo-1,2- |
| | dihydro-1,8-naphthyridin-3-yl)oxy)-N-methylacetamide |
| 156d | rel-2-((6-((5-chloro-2-((6R,7R)-7-((3-(2,6-dioxopiperidin-3-yl)-1-methyl-1H-indazol-6- |
| | yl)amino)-6-methyl-2-azaspiro[3.5]nonan-2-yl)pyrimidin-4-yl)amino)-1-methyl-2-oxo-1,2- |
| | dihydro-1,8-naphthyridin-3-yl)oxy)-N-methylacetamide |
| 157 | 2-((6-((5-chloro-2-(4-((1-(3-(2,6-dioxopiperidin-3-yl)-1-methyl-1H-indazol-7-yl)piperidin- |
| | 4-yl)methyl)piperazin-1-yl)pyrimidin-4-yl)amino)-1-(cyclopropylmethyl)-2-oxo-1,2- |
| | dihydro-1,8-naphthyridin-3-yl)oxy)-N-methylacetamide |
| 158 | 2-((6-((5-chloro-2-(4-(2-(1-(3-(2,6-dioxopiperidin-3-yl)-1-methyl-1H-indazol-6- |
| | yl)piperidin-4-yl)propan-2-yl)piperazin-1-yl)pyrimidin-4-yl)amino)-1-isopropyl-2-oxo-1,2- |
| | dihydro-1,8-naphthyridin-3-yl)oxy)-N-methylacetamide |
| 159 | 2-[[6-[[5-chloro-2-[7-[[3-(2,6-dioxo-3-piperidyl)-1-methyl-indazol-7-yl]amino]-6-methyl-2- |
| | azaspiro[3.5]nonan-2-yl]pyrimidin-4-yl]amino]-1-methyl-2-oxo-1,8-naphthyridin-3-yl]oxy]- |
| | N-methyl-acetamide |
| 159a | rel-2-((6-((5-chloro-2-((6R,7S)-7-((3-(2,6-dioxopiperidin-3-yl)-1-methyl-1H-indazol-7- |
| | yl)amino)-6-methyl-2-azaspiro[3.5]nonan-2-yl)pyrimidin-4-yl)amino)-1-methyl-2-oxo-1,2- |
| | dihydro-1,8-naphthyridin-3-yl)oxy)-N-methylacetamide |
| 159b | rel-2-((6-((5-chloro-2-((6S,7R)-7-((3-(2,6-dioxopiperidin-3-yl)-1-methyl-1H-indazol-7- |
| | yl)amino)-6-methyl-2-azaspiro[3.5]nonan-2-yl)pyrimidin-4-yl)amino)-1-methyl-2-oxo-1,2- |
| | dihydro-1,8-naphthyridin-3-yl)oxy)-N-methylacetamide |
| 160 | 2-((6-((5-chloro-2-(4-((1-(3-(2,6-dioxopiperidin-3-yl)-1-methyl-1H-indazol-7-yl)piperidin- |
| | 4-yl)methyl)piperazin-1-yl)pyridin-4-yl)amino)-1-methyl-2-oxo-1,2-dihydro-1,8- |
| | naphthyridin-3-yl)oxy)-N-methylacetamide |
| 161 | 2-((6-((5-chloro-2-(4-((4-(3-(2,6-dioxopiperidin-3-yl)-1-methyl-1H-indazol-7-yl)piperidin- |
| | 1-yl)methyl)-4-fluoropiperidin-1-yl)pyrimidin-4-yl)amino)-1-isopropyl-2-oxo-1,2-dihydro- |
| | 1,8-naphthyridin-3-yl)oxy)-N-methylacetamide |
| 162 | 2-((6-((5-chloro-2-(7-((1-(3-(2,6-dioxopiperidin-3-yl)-1-methyl-1H-indazol-7-yl)piperidin- |
| | 4-yl)methyl)-3-oxa-7,9-diazabicyclo[3.3.1]nonan-9-yl)pyrimidin-4-yl)amino)-1-isopropyl- |
| | 2-oxo-1,2-dihydro-1,8-naphthyridin-3-yl)oxy)-N-methylacetamide |
| 163 | 2-[[6-[[5-chloro-2-[4-[[1-[3-(2,6-dioxo-3-piperidyl)-1-methyl-indazol-7-yl]-4- |
| | piperidyl]methyl]-3-(trifluoromethyl)piperazin-1-yl]pyrimidin-4-yl]amino]-1-isopropyl-2- |
| | oxo-1,8-naphthyridin-3-yl]oxy]-N-methyl-acetamide |
| 163a | 2-[[6-[[5-chloro-2-[(3R)-4-[[1-[3-(2,6-dioxo-3-piperidyl)-1-methyl-indazol-7-yl]-4- |
| | piperidyl]methyl]-3-(trifluoromethyl)piperazin-1-yl]pyrimidin-4-yl]amino]-1-isopropyl-2- |
| | oxo-1,8-naphthyridin-3-yl]oxy]-N-methyl-acetamide |
| 164 | 2-[[6-[[5-chloro-2-[4-[3-(2,6-dioxo-3-piperidyl)-1-methyl-indazol-6-yl]oxy-1- |
| | piperidyl]pyrimidin-4-yl]amino]-1-isopropyl-2-oxo-1,8-naphthyridin-3-yl]oxy]-N-methyl- |
| | acetamide |
| 165 | 2-[[6-[[5-chloro-2-[4-[3-(2,6-dioxo-3-piperidyl)-1-methyl-indazol-6-yl]piperazin-1- |
| | yl]pyrimidin-4-yl]amino]-1-isopropyl-2-oxo-1,8-naphthyridin-3-yl]oxy]-N-methyl- |
| | acetamide |
| 166 | 2-[[6-[[5-chloro-2-[4-[3-(2,6-dioxo-3-piperidyl)-1-methyl-indazol-7-yl]piperazin-1- |
| | yl]pyrimidin-4-yl]amino]-1-isopropyl-2-oxo-1,8-naphthyridin-3-yl]oxy]-N-methyl- |
| | acetamide |
| 167 | 2-[[6-[[5-chloro-2-[4-[[4-[3-(2,6-dioxo-3-piperidyl)-1-methyl-indazol-6-yl]-1- |
| | piperidyl]methyl]-4-fluoro-1-piperidyl]pyrimidin-4-yl]amino]-1-isopropyl-2-oxo-1,8- |
| | naphthyridin-3-yl]oxy]-N-methyl-acetamide |
| 168 | 2-[[6-[[5-chloro-2-[4-[[1-[3-(2,6-dioxo-3-piperidyl)-1-methyl-indazol-7-yl]-4- |
| | piperidyl]methyl]-2-methyl-piperazin-1-yl]pyrimidin-4-yl]amino]-1-isopropyl-2-oxo-1,8- |
| | naphthyridin-3-yl]oxy]-N-methyl-acetamide |
| 168a | 2-[[6-[[5-chloro-2-[(2S)-4-[[1-[3-(2,6-dioxo-3-piperidyl)-1-methyl-indazol-7-yl]-4- |
| | piperidyl]methyl]-2-methyl-piperazin-1-yl]pyrimidin-4-yl]amino]-1-isopropyl-2-oxo-1,8- |
| | naphthyridin-3-yl]oxy]-N-methyl-acetamide |
| 168b | 2-[[6-[[5-chloro-2-[(2R)-4-[[1-[3-(2,6-dioxo-3-piperidyl)-1-methyl-indazol-7-yl]-4- |
| | piperidyl]methyl]-2-methyl-piperazin-1-yl]pyrimidin-4-yl]amino]-1-isopropyl-2-oxo-1,8- |
| | naphthyridin-3-yl]oxy]-N-methyl-acetamide |
| 169 | 2-[[6-[[5-chloro-2-[4-[[4-[3-(2,6-dioxo-3-piperidyl)-1-methyl-indazol-7-yl]-3-methyl- |
| | piperazin-1-yl]methyl]-1-piperidyl]pyrimidin-4-yl]amino]-1-isopropyl-2-oxo-1,8- |
| | naphthyridin-3-yl]oxy]-N-methyl-acetamide |
| 169a | 2-((6-((5-chloro-2-(4-(((3S)-4-(3-(2,6-dioxopiperidin-3-yl)-1-methyl-1H-indazol-7-yl)-3- |
| | methylpiperazin-1-yl)methyl)piperidin-1-yl)pyrimidin-4-yl)amino)-1-isopropyl-2-oxo-1,2- |
| | dihydro-1,8-naphthyridin-3-yl)oxy)-N-methylacetamide |
| 169b | 2-((6-((5-chloro-2-(4-(((3R)-4-(3-(2,6-dioxopiperidin-3-yl)-1-methyl-1H-indazol-7-yl)-3- |
| | methylpiperazin-1-yl)methyl)piperidin-1-yl)pyrimidin-4-yl)amino)-1-isopropyl-2-oxo-1,2- |
| | dihydro-1,8-naphthyridin-3-yl)oxy)-N-methylacetamide |
| 170 | 2-[[6-[[5-chloro-2-[4-[[1-[3-(2,6-dioxo-3-piperidyl)-1-methyl-indazol-7-yl]-4- |
| | piperidyl]methyl]piperazin-1-yl]pyrimidin-4-yl]amino]-2-oxo-1-(2,2,2-trifluoroethyl)-1,8- |
| | naphthyridin-3-yl]oxy]-N-methyl-acetamide |
| 171 | 2-[[6-[[5-chloro-2-[4-[1-[4-[3-(2,6-dioxo-3-piperidyl)-1-methyl-indazol-6-yl]piperazin-1- |
| | yl]-1-methyl-ethyl]-1-piperidyl]pyrimidin-4-yl]amino]-1-isopropyl-2-oxo-1,8-naphthyridin- |
| | 3-yl]oxy]-N-methyl-acetamide |
| 172 | 2-[[6-[[5-chloro-2-[4-[[1-[3-(2,6-dioxo-3-piperidyl)-1-methyl-indazol-7-yl]-4- |
| | piperidyl]methyl]-3,3-dimethyl-piperazin-1-yl]pyrimidin-4-yl]amino]-1-isopropyl-2-oxo- |
| | 1,8-naphthyridin-3-yl]oxy]-N-methyl-acetamide |
| 173 | 2-[[6-[[5-chloro-2-[3-[[1-[3-(2,6-dioxo-3-piperidyl)-1-methyl-indazol-7-yl]-4- |
| | piperidyl]methyl]-3,6-diazabicyclo[3.1.1]heptan-6-yl]pyrimidin-4-yl]amino]-1-isopropyl-2- |
| | oxo-1,8-naphthyridin-3-yl]oxy]-N-methyl-acetamide |
| 174 | 2-[[6-[[5-chloro-2-[5-[[1-[3-(2,6-dioxo-3-piperidyl)-1-methyl-indazol-7-yl]-4- |
| | piperidyl]methyl]-2,5-diazabicyclo[2.2.1]heptan-2-yl]pyrimidin-4-yl]amino]-1-isopropyl-2- |
| | oxo-1,8-naphthyridin-3-yl]oxy]-N-methyl-acetamide |
| 174a | 2-[[6-[[5-chloro-2-[(1R,4R)-5-[[1-[3-(2,6-dioxo-3-piperidyl)-1-methyl-indazol-7-yl]-4- |
| | piperidyl]methyl]-2,5-diazabicyclo[2.2.1]heptan-2-yl]pyrimidin-4-yl]amino]-1-isopropyl-2- |
| | oxo-1,8-naphthyridin-3-yl]oxy]-N-methyl-acetamide |
| 174b | 2-[[6-[[5-chloro-2-[(1S,4S)-5-[[1-[3-(2,6-dioxo-3-piperidyl)-1-methyl-indazol-7-yl]-4- |
| | piperidyl]methyl]-2,5-diazabicyclo[2.2.1]heptan-2-yl]pyrimidin-4-yl]amino]-1-isopropyl-2- |
| | oxo-1,8-naphthyridin-3-yl]oxy]-N-methyl-acetamide |
| 175 | 2-[[6-[[5-chloro-2-[4-[[5-[3-(2,6-dioxo-3-piperidyl)-1-methyl-indazol-7-yl]-2,5- |
| | diazabicyclo[2.2.1]heptan-2-yl]methyl]-1-piperidyl]pyrimidin-4-yl]amino]-1-isopropyl-2- |
| | oxo-1,8-naphthyridin-3-yl]oxy]-N-methyl-acetamide |
| 175a | 2-[[6-[[5-chloro-2-[4-[[(1R,4R)-5-[3-(2,6-dioxo-3-piperidyl)-1-methyl-indazol-7-yl]-2,5- |
| | diazabicyclo[2.2.1]heptan-2-yl]methyl]-1-piperidyl]pyrimidin-4-yl]amino]-1-isopropyl-2- |
| | oxo-1,8-naphthyridin-3-yl]oxy]-N-methyl-acetamide |
| 175b | 2-[[6-[[5-chloro-2-[4-[[(1S,4S)-5-[3-(2,6-dioxo-3-piperidyl)-1-methyl-indazol-7-yl]-2,5- |
| | diazabicyclo[2.2.1]heptan-2-yl]methyl]-1-piperidyl]pyrimidin-4-yl]amino]-1-isopropyl-2- |
| | oxo-1,8-naphthyridin-3-yl]oxy]-N-methyl-acetamide |
| 176 | 2-[[6-[[5-chloro-2-[4-[[4-[3-(2,6-dioxo-3-piperidyl)-1-methyl-indazol-6-yl]piperazin-1- |
| | yl]methyl]-3-methyl-1-piperidyl]pyrimidin-4-yl]amino]-1-isopropyl-2-oxo-1,8- |
| | naphthyridin-3-yl]oxy]-N-methyl-acetamide |
| 176a | 2-((6-((5-chloro-2-((3S,4S)-4-((4-(3-(2,6-dioxopiperidin-3-yl)-1-methyl-1H-indazol-6-yl) |
| | piperazin-1-yl) methyl)-3-methylpiperidin-1-yl) pyrimidin-4-yl) amino)-1-isopropyl-2-oxo- |
| | 1,2-dihydro-1,8-naphthyridin-3-yl) oxy)-N-methylacetamide |
| 176b | 2-((6-((5-chloro-2-((3R,4S)-4-((4-(3-(2,6-dioxopiperidin-3-yl)-1-methyl-1H-indazol-6- |
| | yl)piperazin-1-yl)methyl)-3-methylpiperidin-1-yl)pyrimidin-4-yl)amino)-1-isopropyl-2-oxo- |
| | 1,2-dihydro-1,8-naphthyridin-3-yl)oxy)-N-methylacetamide |
| 176c | 2-((6-((5-chloro-2-((3R,4R)-4-((4-(3-(2,6-dioxopiperidin-3-yl)-1-methyl-1H-indazol-6- |
| | yl)piperazin-1-yl)methyl)-3-methylpiperidin-1-yl)pyrimidin-4-yl)amino)-1-isopropyl-2-oxo- |
| | 1,2-dihydro-1,8-naphthyridin-3-yl)oxy)-N-methylacetamide |
| 176d | 2-[[6-[[5-chloro-2-[(3S,4R)-4-[[4-[3-(2,6-dioxo-3-piperidyl)-1-methyl-indazol-6- |
| | yl]piperazin-1-yl]methyl]-3-methyl-1-piperidyl]pyrimidin-4-yl]amino]-1-isopropyl-2-oxo- |
| | 1,8-naphthyridin-3-yl]oxy]-N-methyl-acetamide |
| 177 | 2-[[6-[[5-chloro-2-[4-[[4-[3-(2,6-dioxo-3-piperidyl)-1-methyl-indazol-7-yl]piperazin-1- |
| | yl]methyl]-3-methyl-1-piperidyl]pyrimidin-4-yl]amino]-1-isopropyl-2-oxo-1,8- |
| | naphthyridin-3-yl]oxy]-N-methyl-acetamide |
| 177a | 2-((6-((5-chloro-2-((3S,4S)-4-((4-(3-(2,6-dioxopiperidin-3-yl)-1-methyl-1H-indazol-7- |
| | yl)piperazin-1-yl)methyl)-3-methylpiperidin-1-yl)pyrimidin-4-yl)amino)-1-isopropyl-2-oxo- |
| | 1,2-dihydro-1,8-naphthyridin-3-yl)oxy)-N-methylacetamide |
| 177b | 2-[[6-[[5-chloro-2-[(3R,4R)-4-[[4-[3-(2,6-dioxo-3-piperidyl)-1-methyl-indazol-7- |
| | yl]piperazin-1-yl]methyl]-3-methyl-1-piperidyl]pyrimidin-4-yl]amino]-1-isopropyl-2-oxo- |
| | 1,8-naphthyridin-3-yl]oxy]-N-methyl-acetamide |
| 177c | 2-[[6-[[5-chloro-2-[(3S,4R)-4-[[4-[3-(2,6-dioxo-3-piperidyl)-1-methyl-indazol-7- |
| | yl]piperazin-1-yl]methyl]-3-methyl-1-piperidyl]pyrimidin-4-yl]amino]-1-isopropyl-2-oxo- |
| | 1,8-naphthyridin-3-yl]oxy]-N-methyl-acetamide |
| 177d | 2-((6-((5-chloro-2-((3R,4S)-4-((4-(3-(2,6-dioxopiperidin-3-yl)-1-methyl-1H-indazol-7- |
| | yl)piperazin-1-yl)methyl)-3-methylpiperidin-1-yl)pyrimidin-4-yl)amino)-1-isopropyl-2-oxo- |
| | 1,2-dihydro-1,8-naphthyridin-3-yl)oxy)-N-methylacetamide |
| 178 | 2-[[6-[[5-chloro-2-[4-[[4-[3-(2,6-dioxo-3-piperidyl)-1-methyl-indazol-7-yl]piperazin-1- |
| | yl]methyl]-4-hydroxy-1-piperidyl]pyrimidin-4-yl]amino]-1-isopropyl-2-oxo-1,8- |
| | naphthyridin-3-yl]oxy]-N-methyl-acetamide |
| 179 | 2-[[6-[[5-chloro-2-[4-[[1-[3-(2,6-dioxo-3-piperidyl)-1-methyl-indazol-7-yl]-4- |
| | piperidyl]methyl]-2,5-dimethyl-piperazin-1-yl]pyrimidin-4-yl]amino]-1-isopropyl-2-oxo- |
| | 1,8-naphthyridin-3-yl]oxy]-N-methyl-acetamide |
| 179a | 2-[[6-[[5-chloro-2-[(2R,5S)-4-[[1-[3-(2,6-dioxo-3-piperidyl)-1-methyl-indazol-7-yl]-4- |
| | piperidyl]methyl]-2,5-dimethyl-piperazin-1-yl]pyrimidin-4-yl]amino]-1-isopropyl-2-oxo- |
| | 1,8-naphthyridin-3-yl]oxy]-N-methyl-acetamide |
| 179b | 2-[[6-[[5-chloro-2-[(2S,5R)-4-[[1-[3-(2,6-dioxo-3-piperidyl)-1-methyl-indazol-7-yl]-4- |
| | piperidyl]methyl]-2,5-dimethyl-piperazin-1-yl]pyrimidin-4-yl]amino]-1-isopropyl-2-oxo- |
| | 1,8-naphthyridin-3-yl]oxy]-N-methyl-acetamide |
| 180 | 2-[[6-[[5-chloro-2-[7-[[3-(2,6-dioxo-3-piperidyl)-1-methyl-indazol-6-yl]amino]-6-methyl-2- |
| | azaspiro[3.5]nonan-2-yl]pyrimidin-4-yl]amino]-1-isopropyl-2-oxo-1,8-naphthyridin-3- |
| | yl]oxy]-N-methyl-acetamide |
| 180a | rel-2-((6-((5-chloro-2-((6S,7R)-7-((3-(2,6-dioxopiperidin-3-yl)-1-methyl-1H-indazol-6- |
| | yl)amino)-6-methyl-2-azaspiro[3.5]nonan-2-yl)pyrimidin-4-yl)amino)-1-isopropyl-2-oxo- |
| | 1,2-dihydro-1,8-naphthyridin-3-yl)oxy)-N-methylacetamide |
| 180b | rel-2-((6-((5-chloro-2-((6R,7S)-7-((3-(2,6-dioxopiperidin-3-yl)-1-methyl-1H-indazol-6- |
| | yl)amino)-6-methyl-2-azaspiro[3.5]nonan-2-yl)pyrimidin-4-yl)amino)-1-isopropyl-2-oxo- |
| | 1,2-dihydro-1,8-naphthyridin-3-yl)oxy)-N-methylacetamide |
| 180c | rel-2-((6-((5-chloro-2-((6S,7S)-7-((3-(2,6-dioxopiperidin-3-yl)-1-methyl-1H-indazol-6- |
| | yl)amino)-6-methyl-2-azaspiro[3.5]nonan-2-yl)pyrimidin-4-yl)amino)-1-isopropyl-2-oxo- |
| | 1,2-dihydro-1,8-naphthyridin-3-yl)oxy)-N-methylacetamide |
| 180d | rel-2-((6-((5-chloro-2-((6R,7R)-7-((3-(2,6-dioxopiperidin-3-yl)-1-methyl-1H-indazol-6- |
| | yl)amino)-6-methyl-2-azaspiro[3.5]nonan-2-yl)pyrimidin-4-yl)amino)-1-isopropyl-2-oxo- |
| | 1,2-dihydro-1,8-naphthyridin-3-yl)oxy)-N-methylacetamide |
| 181 | 2-[[6-[[5-chloro-2-[7-[3-(2,6-dioxo-3-piperidyl)-1-methyl-indazol-7-yl]oxy-2- |
| | azaspiro[3.5]nonan-2-yl]pyrimidin-4-yl]amino]-1-isopropyl-2-oxo-1,8-naphthyridin-3- |
| | yl]oxy]-N-methyl-acetamide |
| 182 | 2-((6-((5-chloro-2-(7-((3-(2,6-dioxopiperidin-3-yl)-1-methyl-1H-indazol-6-yl)amino)-2- |
| | azaspiro[3.5]nonan-2-yl)pyrimidin-4-yl)amino)-1-isopropyl-2-oxo-1,2-dihydro-1,8- |
| | naphthyridin-3-yl)oxy)-N-methylacetamide |
| 183 | 2-[[6-[[5-chloro-2-[7-[3-(2,6-dioxo-3-piperidyl)-1-methyl-indazol-6-yl]oxy-2- |
| | azaspiro[3.5]nonan-2-yl]pyrimidin-4-yl]amino]-1-isopropyl-2-oxo-1,8-naphthyridin-3- |
| | yl]oxy]-N-methyl-acetamide |
| 184 | 2-[[6-[[5-chloro-2-[7-[[3-(2,6-dioxo-3-piperidyl)-5-fluoro-1-methyl-indazol-6-yl]amino]-2- |
| | azaspiro[3.5]nonan-2-yl]pyrimidin-4-yl]amino]-1-isopropyl-2-oxo-1,8-naphthyridin-3- |
| | yl]oxy]-N-methyl-acetamide |
| 185 | 2-[[6-[[5-chloro-2-[7-[3-(2,6-dioxo-3-piperidyl)-1-methyl-indazol-6-yl]oxy-6-methyl-2- |
| | azaspiro[3.5]nonan-2-yl]pyrimidin-4-yl]amino]-1-isopropyl-2-oxo-1,8-naphthyridin-3- |
| | yl]oxy]-N-methyl-acetamide |
| 185a | rel-2-((6-((5-chloro-2-((6R,7S)-7-((3-(2,6-dioxopiperidin-3-yl)-1-methyl-1H-indazol-6- |
| | yl)oxy)-6-methyl-2-azaspiro[3.5]nonan-2-yl)pyrimidin-4-yl)amino)-1-isopropyl-2-oxo-1,2- |
| | dihydro-1,8-naphthyridin-3-yl)oxy)-N-methylacetamide |
| 185b | rel-2-((6-((5-chloro-2-((6S,7R)-7-((3-(2,6-dioxopiperidin-3-yl)-1-methyl-1H-indazol-6- |
| | yl)oxy)-6-methyl-2-azaspiro[3.5]nonan-2-yl)pyrimidin-4-yl)amino)-1-isopropyl-2-oxo-1,2- |
| | dihydro-1,8-naphthyridin-3-yl)oxy)-N-methylacetamide |
| 185c | rel-2-((6-((5-chloro-2-((6S,7S)-7-((3-(2,6-dioxopiperidin-3-yl)-1-methyl-1H-indazol-6- |
| | yl)oxy)-6-methyl-2-azaspiro[3.5]nonan-2-yl)pyrimidin-4-yl)amino)-1-isopropyl-2-oxo-1,2- |
| | dihydro-1,8-naphthyridin-3-yl)oxy)-N-methylacetamide |
| 185d | rel-2-((6-((5-chloro-2-((6R,7R)-7-((3-(2,6-dioxopiperidin-3-yl)-1-methyl-1H-indazol-6- |
| | yl)oxy)-6-methyl-2-azaspiro[3.5]nonan-2-yl)pyrimidin-4-yl)amino)-1-isopropyl-2-oxo-1,2- |
| | dihydro-1,8-naphthyridin-3-yl)oxy)-N-methylacetamide |
| 186 | 2-((6-((5-chloro-2-(4-((1-(3-(2,6-dioxopiperidin-3-yl)-1-methyl-1H-indazol-7-yl)piperidin- |
| | 4-yl)methyl)piperazin-1-yl)pyrimidin-4-yl)amino)-1-(2-hydroxyethyl)-2-oxo-1,2-dihydro- |
| | 1,8-naphthyridin-3-yl)oxy)-N-methylacetamide |
| 187 | 2-[[6-[[5-chloro-2-[4-[[6-[3-(2,6-dioxo-3-piperidyl)-1-methyl-indazol-7-yl]-3,6- |
| | diazabicyclo[3.1.1]heptan-3-yl]methyl]-1-piperidyl]pyrimidin-4-yl]amino]-1-isopropyl-2- |
| | oxo-1,8-naphthyridin-3-yl]oxy]-N-methyl-acetamide |
| 188 | 2-[[6-[[2-[4-[[4-[3-(2,6-dioxo-3-piperidyl)-1-methyl-indazol-7-yl]-2-methyl-piperazin-1- |
| | yl]methyl]-1-piperidyl]-5-fluoro-pyrimidin-4-yl]amino]-1-isopropyl-2-oxo-1,8- |
| | naphthyridin-3-yl]oxy]-N-methyl-acetamide |
| 188a | 2-[[6-[[2-[4-[[(2R)-4-[3-(2,6-dioxo-3-piperidyl)-1-methyl-indazol-7-yl]-2-methyl-piperazin- |
| | 1-yl]methyl]-1-piperidyl]-5-fluoro-pyrimidin-4-yl]amino]-1-isopropyl-2-oxo-1,8- |
| | naphthyridin-3-yl]oxy]-N-methyl-acetamide |
| 188b | 2-[[6-[[2-[4-[[(2S)-4-[3-(2,6-dioxo-3-piperidyl)-1-methyl-indazol-7-yl]-2-methyl-piperazin- |
| | 1-yl]methyl]-1-piperidyl]-5-fluoro-pyrimidin-4-yl]amino]-1-isopropyl-2-oxo-1,8- |
| | naphthyridin-3-yl]oxy]-N-methyl-acetamide |
| 189 | 2-[[6-[[5-chloro-2-[4-[[4-[3-(2,6-dioxo-3-piperidyl)-1-methyl-indazol-7-yl]-2-methyl- |
| | piperazin-1-yl]methyl]-4-fluoro-1-piperidyl]pyrimidin-4-yl]amino]-1-isopropyl-2-oxo-1,8- |
| | naphthyridin-3-yl]oxy]-N-methyl-acetamide |
| 189a | 2-[[6-[[5-chloro-2-[4-[[(2S)-4-[3-(2,6-dioxo-3-piperidyl)-1-methyl-indazol-7-yl]-2-methyl- |
| | piperazin-1-yl]methyl]-4-fluoro-1-piperidyl]pyrimidin-4-yl]amino]-1-isopropyl-2-oxo-1,8- |
| | naphthyridin-3-yl]oxy]-N-methyl-acetamide |
| 190 | 2-[[6-[[5-chloro-2-[4-[[4-[3-(2,6-dioxo-3-piperidyl)-1-methyl-indazol-6-yl]piperazin-1- |
| | yl]methyl]-1-piperidyl]-4-pyridyl]amino]-1-isopropyl-2-oxo-1,8-naphthyridin-3-yl]oxy]-N- |
| | methyl-acetamide |
| 191 | 2-[[6-[[5-chloro-2-[4-[[1-[3-(2,6-dioxo-3-piperidyl)-1-methyl-indazol-7-yl]-4- |
| | piperidyl]methyl]-4,7-diazaspiro[2.5]octan-7-yl]pyrimidin-4-yl]amino]-1-isopropyl-2-oxo- |
| | 1,8-naphthyridin-3-yl]oxy]-N-methyl-acetamide |
| 192 | 2-[[6-[[5-chloro-2-[4-[[4-[3-(2,6-dioxo-3-piperidyl)-1-methyl-indazol-7-yl]-2-methyl- |
| | piperazin-1-yl]methyl]-1-piperidyl]pyrimidin-4-yl]amino]-1-isopropyl-2-oxo-1,8- |
| | naphthyridin-3-yl]oxy]-N-methyl-acetamide |
| 192a | 2-[[6-[[5-chloro-2-[4-[[(2S)-4-[3-(2,6-dioxo-3-piperidyl)-1-methyl-indazol-7-yl]-2-methyl- |
| | piperazin-1-yl]methyl]-1-piperidyl]pyrimidin-4-yl]amino]-1-isopropyl-2-oxo-1,8- |
| | naphthyridin-3-yl]oxy]-N-methyl-acetamide |
| 192b | 2-[[6-[[5-chloro-2-[4-[[(2R)-4-[3-(2,6-dioxo-3-piperidyl)-1-methyl-indazol-7-yl]-2-methyl- |
| | piperazin-1-yl]methyl]-1-piperidyl]pyrimidin-4-yl]amino]-1-isopropyl-2-oxo-1,8- |
| | naphthyridin-3-yl]oxy]-N-methyl-acetamide |
| 193 | 2-[[6-[[5-chloro-2-[4-[1-[4-[3-(2,6-dioxo-3-piperidyl)-1-methyl-indazol-7-yl]piperazin-1- |
| | yl]ethyl]-1-piperidyl]pyrimidin-4-yl]amino]-1-isopropyl-2-oxo-1,8-naphthyridin-3-yl]oxy]- |
| | N-methyl-acetamide |
| 193a | rel-2-((6-((5-chloro-2-(4-((1R)-1-(4-(3-(2,6-dioxopiperidin-3-yl)-1-methyl-1H-indazol-7- |
| | yl)piperazin-1-yl)ethyl)piperidin-1-yl)pyrimidin-4-yl)amino)-1-isopropyl-2-oxo-1,2- |
| | dihydro-1,8-naphthyridin-3-yl)oxy)-N-methylacetamide |
| 193b | rel-2-((6-((5-chloro-2-(4-((1S)-1-(4-(3-(2,6-dioxopiperidin-3-yl)-1-methyl-1H-indazol-7- |
| | yl)piperazin-1-yl)ethyl)piperidin-1-yl)pyrimidin-4-yl)amino)-1-isopropyl-2-oxo-1,2- |
| | dihydro-1,8-naphthyridin-3-yl)oxy)-N-methylacetamide |
| 194 | 2-[[6-[[5-chloro-2-[4-[1-[1-[3-(2,6-dioxo-3-piperidyl)-1-methyl-indazol-7-yl]-4-piperidyl]- |
| | 1-hydroxy-ethyl]-1-piperidyl]pyrimidin-4-yl]amino]-1-isopropyl-2-oxo-1,8-naphthyridin-3- |
| | yl]oxy]-N-methyl-acetamide |
| 194a | rel-2-((6-((5-chloro-2-(4-((1S)-1-(1-(3-(2,6-dioxopiperidin-3-yl)-1-methyl-1H-indazol-7- |
| | yl)piperidin-4-yl)-1-hydroxyethyl)piperidin-1-yl)pyrimidin-4-yl)amino)-1-isopropyl-2-oxo- |
| | 1,2-dihydro-1,8-naphthyridin-3-yl)oxy)-N-methylacetamide |
| 194b | rel-2-((6-((5-chloro-2-(4-((1R)-1-(1-(3-(2,6-dioxopiperidin-3-yl)-1-methyl-1H-indazol-7- |
| | yl)piperidin-4-yl)-1-hydroxyethyl)piperidin-1-yl)pyrimidin-4-yl)amino)-1-isopropyl-2-oxo- |
| | 1,2-dihydro-1,8-naphthyridin-3-yl)oxy)-N-methylacetamide |
| 195 | 2-[[6-[[5-chloro-2-[4-[1-[1-[3-(2,6-dioxo-3-piperidyl)-1-methyl-indazol-7-yl]-4- |
| | piperidyl]ethyl]piperazin-1-yl]pyrimidin-4-yl]amino]-1-isopropyl-2-oxo-1,8-naphthyridin-3- |
| | yl]oxy]-N-methyl-acetamide |
| 195a | rel-2-((6-((5-chloro-2-(4-((1S)-1-(1-(3-(2,6-dioxopiperidin-3-yl)-1-methyl-1H-indazol-7- |
| | yl)piperidin-4-yl)ethyl)piperazin-1-yl)pyrimidin-4-yl)amino)-1-isopropyl-2-oxo-1,2- |
| | dihydro-1,8-naphthyridin-3-yl)oxy)-N-methylacetamide |
| 195b | rel-2-((6-((5-chloro-2-(4-((1R)-1-(1-(3-(2,6-dioxopiperidin-3-yl)-1-methyl-1H-indazol-7- |
| | yl)piperidin-4-yl)ethyl)piperazin-1-yl)pyrimidin-4-yl)amino)-1-isopropyl-2-oxo-1,2- |
| | dihydro-1,8-naphthyridin-3-yl)oxy)-N-methylacetamide |
| 196 | 2-((6-((5-chloro-2-(4-((4-(3-(2,6-dioxopiperidin-3-yl)-1-methyl-1H-indazol-6-yl)-2,2- |
| | dimethylpiperazin-1-yl)methyl) piperidin-1-yl)pyrimidin-4-yl)amino)-1-isopropyl-2-oxo- |
| | 1,2-dihydro-1,8-naphthyridin-3-yl)oxy)-N-methylacetamide |
| 197 | 2-[[6-[[5-chloro-2-[4-[[4-[3-(2,6-dioxo-3-piperidyl)-1-methyl-indazol-7-yl]-2,6-dimethyl- |
| | piperazin-1-yl]methyl]-1-piperidyl]pyrimidin-4-yl]amino]-1-isopropyl-2-oxo-1,8- |
| | naphthyridin-3-yl]oxy]-N-methyl-acetamide |
| 197a | 2-[[6-[[5-chloro-2-[4-[[(2R,6S)-4-[3-(2,6-dioxo-3-piperidyl)-1-methyl-indazol-7-yl]-2,6- |
| | dimethyl-piperazin-1-yl]methyl]-1-piperidyl]pyrimidin-4-yl]amino]-1-isopropyl-2-oxo-1,8- |
| | naphthyridin-3-yl]oxy]-N-methyl-acetamide |
| 197b | 2-[[6-[[5-chloro-2-[4-[[(2S,6S)-4-[3-(2,6-dioxo-3-piperidyl)-1-methyl-indazol-7-yl]-2,6- |
| | dimethyl-piperazin-1-yl]methyl]-1-piperidyl]pyrimidin-4-yl]amino]-1-isopropyl-2-oxo-1,8- |
| | naphthyridin-3-yl]oxy]-N-methyl-acetamide |
| 198 | 2-[[6-[[5-chloro-2-[7-[[3-(2,6-dioxo-3-piperidyl)-4-fluoro-1-methyl-indazol-7-yl]amino]-2- |
| | azaspiro[3.5]nonan-2-yl]pyrimidin-4-yl]amino]-1-isopropyl-2-oxo-1,8-naphthyridin-3- |
| | yl]oxy]-N-methyl-acetamide |
| 199 | 2-[[6-[[5-chloro-2-[9-[[3-(2,6-dioxo-3-piperidyl)-1-methyl-indazol-6-yl]amino]-3- |
| | azaspiro[5.5]undecan-3-yl]pyrimidin-4-yl]amino]-1-isopropyl-2-oxo-1,8-naphthyridin-3- |
| | yl]oxy]-N-methyl-acetamide |
| 200 | 2-[[6-[[5-chloro-2-[4-[[4-[3-(2,6-dioxo-3-piperidyl)-1-methyl-indazol-7-yl]-2,2-dimethyl- |
| | piperazin-1-yl]methyl]-1-piperidyl]pyrimidin-4-yl]amino]-1-isopropyl-2-oxo-1,8- |
| | naphthyridin-3-yl]oxy]-N-methyl-acetamide |
| 201 | 2-[[6-[[5-chloro-2-[4-[1-[1-[3-(2,6-dioxo-3-piperidyl)-1-methyl-indazol-6-yl]-4-piperidyl]- |
| | 1-hydroxy-ethyl]-1-piperidyl]pyrimidin-4-yl]amino]-1-isopropyl-2-oxo-1,8-naphthyridin-3- |
| | yl]oxy]-N-methyl-acetamide |
| 201a | rel-2-((6-((5-chloro-2-(4-((1S)-1-(1-(3-(2,6-dioxopiperidin-3-yl)-1-methyl-1H-indazol-6- |
| | yl)piperidin-4-yl)-1-hydroxyethyl)piperidin-1-yl)pyrimidin-4-yl)amino)-1-isopropyl-2-oxo- |
| | 1,2-dihydro-1,8-naphthyridin-3-yl)oxy)-N-methylacetamide |
| 201b | rel-2-((6-((5-chloro-2-(4-((1R)-1-(1-(3-(2,6-dioxopiperidin-3-yl)-1-methyl-1H-indazol-6- |
| | yl)piperidin-4-yl)-1-hydroxyethyl)piperidin-1-yl)pyrimidin-4-yl)amino)-1-isopropyl-2-oxo- |
| | 1,2-dihydro-1,8-naphthyridin-3-yl)oxy)-N-methylacetamide |
| 202 | 2-[[6-[[5-chloro-2-[6-[[3-(2,6-dioxo-3-piperidyl)-1-methyl-indazol-6-yl]amino]-2- |
| | azaspiro[3.3]heptan-2-yl]pyrimidin-4-yl]amino]-1-isopropyl-2-oxo-1,8-naphthyridin-3- |
| | yl]oxy]-N-methyl-acetamide |
| 203 | 2-[[6-[[5-chloro-2-[4-[[4-[3-(2,6-dioxo-3-piperidyl)-1-methyl-indazol-7-yl]-1- |
| | piperidyl]methyl]-4-hydroxy-1-piperidyl]pyrimidin-4-yl]amino]-1-isopropyl-2-oxo-1,8- |
| | naphthyridin-3-yl]oxy]-N-methyl-acetamide |
| 204 | 2-((6-((5-chloro-2-(9-((4-(3-(2,6-dioxopiperidin-3-yl)-1-methyl-1H-indazol-6-yl)piperazin- |
| | 1-yl)methyl)-3-azaspiro[5.5]undecan-3-yl)pyrimidin-4-yl)amino)-1-isopropyl-2-oxo-1,2- |
| | dihydro-1,8-naphthyridin-3-yl)oxy)-N-methylacetamide |
| 205 | 2-((6-((5-chloro-2-(7-((4-(3-(2,6-dioxopiperidin-3-yl)-1-methyl-1H-indazol-6-yl)piperazin- |
| | 1-yl)methyl)-2-azaspiro[3.5]nonan-2-yl)pyrimidin-4-yl)amino)-1-isopropyl-2-oxo-1,2- |
| | dihydro-1,8-naphthyridin-3-yl)oxy)-N-methylacetamide |
| 206 | 2-[[6-[[5-chloro-2-[[2-[3-(2,6-dioxo-3-piperidyl)-1-methyl-indazol-6-yl]-2- |
| | azaspiro[3.5]nonan-7-yl]amino]pyrimidin-4-yl]amino]-1-isopropyl-2-oxo-1,8-naphthyridin- |
| | 3-yl]oxy]-N-methyl-acetamide |
| 207 | 2-[[6-[[5-chloro-2-[6-[[3-(2,6-dioxo-3-piperidyl)-1-methyl-indazol-6-yl]-methyl-amino]-2- |
| | azaspiro[3.3]heptan-2-yl]pyrimidin-4-yl]amino]-1-isopropyl-2-oxo-1,8-naphthyridin-3- |
| | yl]oxy]-N-methyl-acetamide |
| 208 | 2-[[6-[[5-chloro-2-[4-[[1-[3-(2,6-dioxo-3-piperidyl)-1-methyl-indazol-7-yl]-4- |
| | piperidyl]methyl]-3-(1-hydroxy-1-methyl-ethyl)piperazin-1-yl]pyrimidin-4-yl]amino]-1- |
| | isopropyl-2-oxo-1,8-naphthyridin-3-yl]oxy]-N-methyl-acetamide |
| 208a | 2-[[6-[[5-chloro-2-[(3S)-4-[[1-[3-(2,6-dioxo-3-piperidyl)-1-methyl-indazol-7-yl]-4- |
| | piperidyl]methyl]-3-(1-hydroxy-1-methyl-ethyl)piperazin-1-yl]pyrimidin-4-yl]amino]-1- |
| | isopropyl-2-oxo-1,8-naphthyridin-3-yl]oxy]-N-methyl-acetamide |
| 208b | 2-[[6-[[5-chloro-2-[(3R)-4-[1-[3-(2,6-dioxo-3-piperidyl)-1-methyl-indazol-7-yl]-4- |
| | piperidyl]methyl]-3-(1-hydroxy-1-methyl-ethyl)piperazin-1-yl]pyrimidin-4-yl]amino]-1- |
| | isopropyl-2-oxo-1,8-naphthyridin-3-yl]oxy]-N-methyl-acetamide |
| 209 | 2-[[6-[[5-chloro-2-[4-[[3-[3-(2,6-dioxo-3-piperidyl)-1-methyl-indazol-7-yl]-3,6- |
| | diazabicyclo[3.1.1]heptan-6-yl]methyl]-1-piperidyl]pyrimidin-4-yl]amino]-1-isopropyl-2- |
| | oxo-1,8-naphthyridin-3-yl]oxy]-N-methyl-acetamide |
| 210 | 2-[[6-[[2-[4-[[4-[3-(2,6-dioxo-3-piperidyl)-1-methyl-indazol-7-yl]-3-methyl-piperazin-1- |
| | yl]methyl]-1-piperidyl]-5-fluoro-pyrimidin-4-yl]amino]-1-isopropyl-2-oxo-1,8- |
| | naphthyridin-3-yl]oxy]-N-methyl-acetamide |
| 210a | 2-[[6-[[2-[4-[[(3S)-4-[3-(2,6-dioxo-3-piperidyl)-1-methyl-indazol-7-yl]-3-methyl-piperazin- |
| | 1-yl]methyl]-1-piperidyl]-5-fluoro-pyrimidin-4-yl]amino]-1-isopropyl-2-oxo-1,8- |
| | naphthyridin-3-yl]oxy]-N-methyl-acetamide |
| 210b | 2-[[6-[[2-[4-[[(3R)-4-[3-(2,6-dioxo-3-piperidyl)-1-methyl-indazol-7-yl]-3-methyl-piperazin- |
| | 1-yl]methyl]-1-piperidyl]-5-fluoro-pyrimidin-4-yl]amino]-1-isopropyl-2-oxo-1,8- |
| | naphthyridin-3-yl]oxy]-N-methyl-acetamide |
| 211 | 2-[[6-[[5-chloro-2-[4-[4-[3-(2,6-dioxo-3-piperidyl)-1-methyl-indazol-6-yl]piperazine-1- |
| | carbonyl]-1-piperidyl]pyrimidin-4-yl]amino]-1-isopropyl-2-oxo-1,8-naphthyridin-3-yl]oxy]- |
| | N-methyl-acetamide |
| 212 | 2-((6-((5-chloro-2-(4-((4-(3-(2,6-dioxopiperidin-3-yl)-1-methyl-1H-indazol-6-yl)piperidin- |
| | 1-yl)methyl)piperidin-1-yl)pyrimidin-4-yl)amino)-1-isopropyl-2-oxo-1,2-dihydro-1,8- |
| | naphthyridin-3-yl)oxy)-N-methylacetamide |
| 213 | 2-[[6-[[5-chloro-2-[6-[3-(2,6-dioxo-3-piperidyl)-1-methyl-indazol-6-yl]oxy-2- |
| | azaspiro[3.3]heptan-2-yl]pyrimidin-4-yl]amino]-1-isopropyl-2-oxo-1,8-naphthyridin-3- |
| | yl]oxy]-N-methyl-acetamide |
| 214 | 2-((6-((5-chloro-2-(4-((4-(3-(2,6-dioxopiperidin-3-yl)-1-methyl-1H-indazol-6-yl)piperazin- |
| | 1-yl)methyl)piperidin-1-yl)pyrimidin-4-yl)amino)-1-isopropyl-2-oxo-1,2-dihydro-1,8- |
| | naphthyridin-3-yl)oxy)-N-methylacetamide |
| 215 | 2-[[6-[[5-chloro-2-[4-[[4-[3-(2,6-dioxo-3-piperidyl)-1-methyl-indazol-6-yl]-2-methyl- |
| | piperazin-1-yl]methyl]-1-piperidyl]pyrimidin-4-yl]amino]-1-isopropyl-2-oxo-1,8- |
| | naphthyridin-3-yl]oxy]-N-methyl-acetamide |
| 215a | 2-((6-((5-chloro-2-(4-(((2R)-4-(3-(2,6-dioxopiperidin-3-yl)-1-methyl-1H-indazol-6-yl)-2- |
| | methylpiperazin-1-yl)methyl)piperidin-1-yl)pyrimidin-4-yl)amino)-1-isopropyl-2-oxo-1,2- |
| | dihydro-1,8-naphthyridin-3-yl)oxy)-N-methylacetamide |
| 215b | 2-((6-((5-chloro-2-(4-(((2S)-4-(3-(2,6-dioxopiperidin-3-yl)-1-methyl-1H-indazol-6-yl)-2- |
| | methylpiperazin-1-yl)methyl)piperidin-1-yl)pyrimidin-4-yl)amino)-1-isopropyl-2-oxo-1,2- |
| | dihydro-1,8-naphthyridin-3-yl)oxy)-N-methylacetamide |
| 216 | 2-[[6-[[5-chloro-2-[4-[[1-[3-(2,6-dioxo-3-piperidyl)-1-methyl-indazol-6-yl]pyrrolidin-3- |
| | yl]methyl]-3-methyl-piperazin-1-yl]pyrimidin-4-yl]amino]-1-isopropyl-2-oxo-1,8- |
| | naphthyridin-3-yl]oxy]-N-methyl-acetamide |
| 216a | 2-[[6-[[5-chloro-2-[(3S)-4-[[(3R)-1-[3-(2,6-dioxo-3-piperidyl)-1-methyl-indazol-6- |
| | yl]pyrrolidin-3-yl]methyl]-3-methyl-piperazin-1-yl]pyrimidin-4-yl]amino]-1-isopropyl-2- |
| | oxo-1,8-naphthyridin-3-yl]oxy]-N-methyl-acetamide |
| 216b | 2-((6-((5-Chloro-2-((3S)-4-(((3S)-1-(3-(2,6-dioxopiperidin-3-yl)-1-methyl-1H-indazol-6- |
| | yl)pyrrolidin-3-yl)methyl)-3-methylpiperazin-1-yl)pyrimidin-4-yl)amino)-1-isopropyl-2- |
| | oxo-1,2-dihydro-1,8-naphthyridin-3-yl)oxy)-N-methylacetamide |
| 216c | 2-[[6-[[5-chloro-2-[(3R)-4-[[(3R)-1-[3-(2,6-dioxo-3-piperidyl)-1-methyl-indazol-6- |
| | yl]pyrrolidin-3-yl]methyl]-3-methyl-piperazin-1-yl]pyrimidin-4-yl]amino]-1-isopropyl-2- |
| | oxo-1,8-naphthyridin-3-yl]oxy]-N-methyl-acetamide |
| 216d | 2-[[6-[[5-chloro-2-[(3R)-4-[[(3S)-1-[3-(2,6-dioxo-3-piperidyl)-1-methyl-indazol-6- |
| | yl]pyrrolidin-3-yl]methyl]-3-methyl-piperazin-1-yl]pyrimidin-4-yl]amino]-1-isopropyl-2- |
| | oxo-1,8-naphthyridin-3-yl]oxy]-N-methyl-acetamide |
| 217 | 2-[[6-[[5-chloro-2-[4-[[4-[3-(2,6-dioxo-3-piperidyl)-1-methyl-indazol-7-yl]-3-methyl- |
| | piperazin-1-yl]methyl]-1-piperidyl]-4-pyridyl]amino]-1-isopropyl-2-oxo-1,8-naphthyridin- |
| | 3-yl]oxy]-N-methyl-acetamide |
| 217a | 2-[[6-[[5-chloro-2-[4-[[(3R)-4-[3-(2,6-dioxo-3-piperidyl)-1-methyl-indazol-7-yl]-3-methyl- |
| | piperazin-1-yl]methyl]-1-piperidyl]-4-pyridyl]amino]-1-isopropyl-2-oxo-1,8-naphthyridin- |
| | 3-yl]oxy]-N-methyl-acetamide |
| 217b | 2-[[6-[[5-chloro-2-[4-[[(3S)-4-[3-(2,6-dioxo-3-piperidyl)-1-methyl-indazol-7-yl]-3-methyl- |
| | piperazin-1-yl]methyl]-1-piperidyl]-4-pyridyl]amino]-1-isopropyl-2-oxo-1,8-naphthyridin- |
| | 3-yl]oxy]-N-methyl-acetamide |
| 218 | 2-[[6-[[5-chloro-2-[3-[[4-[3-(2,6-dioxo-3-piperidyl)-1-methyl-indazol-6-yl]piperazin-1- |
| | yl]methyl]pyrrolidin-1-yl]pyrimidin-4-yl]amino]-1-isopropyl-2-oxo-1,8-naphthyridin-3- |
| | yl]oxy]-N-methyl-acetamide |
| 218a | 2-[[6-[[5-chloro-2-[(3S)-3-[[4-[3-(2,6-dioxo-3-piperidyl)-1-methyl-indazol-6-yl]piperazin-1- |
| | yl]methyl]pyrrolidin-1-yl]pyrimidin-4-yl]amino]-1-isopropyl-2-oxo-1,8-naphthyridin-3- |
| | yl]oxy]-N-methyl-acetamide |
| 218b | 2-[[6-[[5-chloro-2-[(3R)-3-[[4-[3-(2,6-dioxo-3-piperidyl)-1-methyl-indazol-6-yl]piperazin- |
| | 1-yl]methyl]pyrrolidin-1-yl]pyrimidin-4-yl]amino]-1-isopropyl-2-oxo-1,8-naphthyridin-3- |
| | yl]oxy]-N-methyl-acetamide |
| 219 | 2-[[6-[[5-chloro-2-[4-[4-[3-(2,6-dioxo-3-piperidyl)-1-methyl-indazol-6-yl]piperazine-1- |
| | carbonyl]piperazin-1-yl]pyrimidin-4-yl]amino]-1-isopropyl-2-oxo-1,8-naphthyridin-3- |
| | yl]oxy]-N-methyl-acetamide |
| 220 | 2-[[6-[[5-chloro-2-[4-[4-[3-(2,6-dioxo-3-piperidyl)-1-methyl-indazol-6-yl]piperidine-1- |
| | carbonyl]piperazin-1-yl]pyrimidin-4-yl]amino]-1-isopropyl-2-oxo-1,8-naphthyridin-3- |
| | yl]oxy]-N-methyl-acetamide |
| 221 | 2-[[6-[[5-chloro-2-[7-[[3-(2,6-dioxo-3-piperidyl)-1-methyl-indazol-6-yl]amino]-6- |
| | (trifluoromethyl)-2-azaspiro[3.5]nonan-2-yl]pyrimidin-4-yl]amino]-1-isopropyl-2-oxo-1,8- |
| | naphthyridin-3-yl]oxy]-N-methyl-acetamide |
| 221a | rel-2-((6-((5-chloro-2-((6S,7R)-7-((3-(2,6-dioxopiperidin-3-yl)-1-methyl-1H-indazol-6- |
| | yl)amino)-6-(trifluoromethyl)-2-azaspiro[3.5]nonan-2-yl)pyrimidin-4-yl)amino)-1- |
| | isopropyl-2-oxo-1,2-dihydro-1,8-naphthyridin-3-yl)oxy)-N-methylacetamide |
| 221b | rel-2-((6-((5-chloro-2-((6S,7S)-7-((3-(2,6-dioxopiperidin-3-yl)-1-methyl-1H-indazol-6- |
| | yl)amino)-6-(trifluoromethyl)-2-azaspiro[3.5]nonan-2-yl)pyrimidin-4-yl)amino)-1- |
| | isopropyl-2-oxo-1,2-dihydro-1,8-naphthyridin-3-yl)oxy)-N-methylacetamide |
| 221c | rel-2-((6-((5-chloro-2-((6R,7R)-7-((3-(2,6-dioxopiperidin-3-yl)-1-methyl-1H-indazol-6- |
| | yl)amino)-6-(trifluoromethyl)-2-azaspiro[3.5]nonan-2-yl)pyrimidin-4-yl)amino)-1- |
| | isopropyl-2-oxo-1,2-dihydro-1,8-naphthyridin-3-yl)oxy)-N-methylacetamide |
| 221d | rel-2-((6-((5-chloro-2-((6R,7S)-7-((3-(2,6-dioxopiperidin-3-yl)-1-methyl-1H-indazol-6- |
| | yl)amino)-6-(trifluoromethyl)-2-azaspiro[3.5]nonan-2-yl)pyrimidin-4-yl)amino)-1- |
| | isopropyl-2-oxo-1,2-dihydro-1,8-naphthyridin-3-yl)oxy)-N-methylacetamide |
| 222 | 2-[[6-[[5-chloro-2-[7-[[3-(2,6-dioxo-3-piperidyl)-6-fluoro-1-methyl-indazol-7-yl]amino]-2- |
| | azaspiro[3.5]nonan-2-yl]pyrimidin-4-yl]amino]-1-isopropyl-2-oxo-1,8-naphthyridin-3- |
| | yl]oxy]-N-methyl-acetamide |
| 223 | 2-[[6-[[5-chloro-2-[4-[4-[3-(2,6-dioxo-3-piperidyl)-1-methyl-indazol-7-yl]piperazine-1- |
| | carbonyl]piperazin-1-yl]pyrimidin-4-yl]amino]-1-isopropyl-2-oxo-1,8-naphthyridin-3- |
| | yl]oxy]-N-methyl-acetamide |
| 224 | 2-[[6-[[5-chloro-2-[4-[4-[3-(2,6-dioxo-3-piperidyl)-1-methyl-indazol-7-yl]piperidine-1- |
| | carbonyl]piperazin-1-yl]pyrimidin-4-yl]amino]-1-isopropyl-2-oxo-1,8-naphthyridin-3- |
| | yl]oxy]-N-methyl-acetamide |
| 225 | 2-[[6-[[5-chloro-2-[4-[[1-[3-(2,6-dioxo-3-piperidyl)-1-methyl-indazol-7-yl]-4- |
| | piperidyl]oxy]-1-piperidyl]pyrimidin-4-yl]amino]-1-isopropyl-2-oxo-1,8-naphthyridin-3- |
| | yl]oxy]-N-methyl-acetamide |
| 226 | 2-[[6-[[5-chloro-2-[4-[3-[4-[3-(2,6-dioxo-3-piperidyl)-1-methyl-indazol-6-yl]piperazin-1- |
| | yl]cyclobutoxy]-1-piperidyl]pyrimidin-4-yl]amino]-1-isopropyl-2-oxo-1,8-naphthyridin-3- |
| | yl]oxy]-N-methyl-acetamide |
| 226a | 2-((6-((5-chloro-2-(4-((1r,3r)-3-(4-(3-(2,6-dioxopiperidin-3-yl)-1-methyl-1H-indazol-6- |
| | yl)piperazin-1-yl)cyclobutoxy)piperidin-1-yl)pyrimidin-4-yl) amino)-1-isopropyl-2-oxo-1,2- |
| | dihydro-1,8-naphthyridin-3-yl)oxy)-N-methylacetamide |
| 227 | 2-[[6-[[5-chloro-2-[4-[3-[[1-[3-(2,6-dioxo-3-piperidyl)-1-methyl-indazol-6-yl]-4- |
| | piperidyl]oxy]cyclobutyl]piperazin-1-yl]pyrimidin-4-yl]amino]-1-isopropyl-2-oxo-1,8- |
| | naphthyridin-3-yl]oxy]-N-methyl-acetamide |
| 227a | 2-((6-((5-chloro-2-(4-((1r,3r)-3-((1-(3-(2,6-dioxopiperidin-3-yl)-1-methyl-1H-indazol-6- |
| | yl)piperidin-4-yl)oxy)cyclobutyl)piperazin-1-yl)pyrimidin-4-yl)amino)-1-isopropyl-2-oxo- |
| | 1,2-dihydro-1,8-naphthyridin-3-yl)oxy)-N-methylacetamide |
| 228 | 2-[[6-[[5-chloro-2-[4-[[1-[3-(2,6-dioxo-3-piperidyl)-1-methyl-indazol-6-yl]pyrrolidin-3- |
| | yl]methyl]piperazin-1-yl]pyrimidin-4-yl]amino]-1-isopropyl-2-oxo-1,8-naphthyridin-3- |
| | yl]oxy]-N-methyl-acetamide |
| 228a | 2-[[6-[[5-chloro-2-[4-[[(3S)-1-[3-(2,6-dioxo-3-piperidyl)-1-methyl-indazol-6-yl]pyrrolidin- |
| | 3-yl]methyl]piperazin-1-yl]pyrimidin-4-yl]amino]-1-isopropyl-2-oxo-1,8-naphthyridin-3- |
| | yl]oxy]-N-methyl-acetamide |
| 228b | 2-((6-((5-chloro-2-(4-(((3R)-1-(3-(2,6-dioxopiperidin-3-yl)-1-methyl-1H-indazol-6- |
| | yl)pyrrolidin-3-yl)methyl)piperazin-1-yl)pyrimidin-4-yl)amino)-1-isopropyl-2-oxo-1,2- |
| | dihydro-1,8-naphthyridin-3-yl)oxy)-N-methylacetamide |
| 229 | 2-[[6-[[5-chloro-2-[2-[4-[3-(2,6-dioxo-3-piperidyl)-1-methyl-indazol-6-yl]piperazin-1-yl]-7- |
| | azaspiro[3.5]nonan-7-yl]pyrimidin-4-yl]amino]-1-isopropyl-2-oxo-1,8-naphthyridin-3- |
| | yl]oxy]-N-methyl-acetamide |
| 230 | 2-[[6-[[5-chloro-2-[2-[4-[3-(2,6-dioxo-3-piperidyl)-1-methyl-indazol-7-yl]piperazin-1-yl]-7- |
| | azaspiro [3.5]nonan-7-yl]pyrimidin-4-yl]amino]-1-isopropyl-2-oxo-1,8-naphthyridin-3- |
| | yl]oxy]-N-methyl-acetamide |
| 231 | 2-[[6-[[5-chloro-2-[4-[[4-[3-(2,6-dioxo-3-piperidyl)-1-methyl-indazol-7-yl]-2- |
| | (hydroxymethyl)piperazin-1-yl]methyl]-1-piperidyl]pyrimidin-4-yl]amino]-1-isopropyl-2- |
| | oxo-1,8-naphthyridin-3-yl]oxy]-N-methyl-acetamide |
| 231a | 2-[[6-[[5-chloro-2-[4-[[(2S)-4-[3-(2,6-dioxo-3-piperidyl)-1-methyl-indazol-7-yl]-2- |
| | (hydroxymethyl)piperazin-1-yl]methyl]-1-piperidyl]pyrimidin-4-yl]amino]-1-isopropyl-2- |
| | oxo-1,8-naphthyridin-3-yl]oxy]-N-methyl-acetamide |
| 231b | 2-[[6-[[5-chloro-2-[4-[[(2R)-4-[3-(2,6-dioxo-3-piperidyl)-1-methyl-indazol-7-yl]-2- |
| | (hydroxymethyl)piperazin-1-yl]methyl]-1-piperidyl]pyrimidin-4-yl]amino]-1-isopropyl-2- |
| | oxo-1,8-naphthyridin-3-yl]oxy]-N-methyl-acetamide |
| 232 | 2-[[6-[[5-chloro-2-[4-[[4-[3-(2,6-dioxo-3-piperidyl)-1-methyl-indazol-6-yl]-2- |
| | (hydroxymethyl)piperazin-1-yl]methyl]-1-piperidyl]pyrimidin-4-yl]amino]-1-isopropyl-2- |
| | oxo-1,8-naphthyridin-3-yl]oxy]-N-methyl-acetamide |
| 232a | 2-[[6-[[5-chloro-2-[4-[[(2S)-4-[3-(2,6-dioxo-3-piperidyl)-1-methyl-indazol-6-yl]-2- |
| | (hydroxymethyl)piperazin-1-yl]methyl]-1-piperidyl]pyrimidin-4-yl]amino]-1-isopropyl-2- |
| | oxo-1,8-naphthyridin-3-yl]oxy]-N-methyl-acetamide |
| 232b | 2-[[6-[[5-chloro-2-[4-[[(2R)-4-[3-(2,6-dioxo-3-piperidyl)-1-methyl-indazol-6-yl]-2- |
| | (hydroxymethyl)piperazin-1-yl]methyl]-1-piperidyl]pyrimidin-4-yl]amino]-1-isopropyl-2- |
| | oxo-1,8-naphthyridin-3-yl]oxy]-N-methyl-acetamide |
| 233 | 2-[[6-[[5-chloro-2-[4-[[1-[3-(2,6-dioxo-3-piperidyl)-1-methyl-indazol-7-yl]-4- |
| | piperidyl]methyl]-3-(hydroxymethyl)piperazin-1-yl]-4-pyridyl]amino]-1-isopropyl-2-oxo- |
| | 1,8-naphthyridin-3-yl]oxy]-N-methyl-acetamide |
| 233a | 2-[[6-[[5-chloro-2-[(3S)-4-[[1-[3-(2,6-dioxo-3-piperidyl)-1-methyl-indazol-7-yl]-4- |
| | piperidyl]methyl]-3-(hydroxymethyl)piperazin-1-yl]-4-pyridyl]amino]-1-isopropyl-2-oxo- |
| | 1,8-naphthyridin-3-yl]oxy]-N-methyl-acetamide |
| 235 | 2-[[6-[[5-chloro-2-[4-[[4-[3-(2,6-dioxo-3-piperidyl)-1-methyl-indazol-6-yl]-1,4-diazepan-1- |
| | yl]methyl]-1-piperidyl]pyrimidin-4-yl]amino]-1-isopropyl-2-oxo-1,8-naphthyridin-3- |
| | yl]oxy]-N-methyl-acetamide |
| 236 | 2-[[6-[[5-chloro-2-[4-[[4-[3-(2,6-dioxo-3-piperidyl)-1-methyl-indazol-7-yl]-1,4-diazepan-1- |
| | yl]methyl]-1-piperidyl]pyrimidin-4-yl]amino]-1-isopropyl-2-oxo-1,8-naphthyridin-3- |
| | yl]oxy]-N-methyl-acetamide |
| 237 | 2-((6-((5-chloro-2-(9-((4-(3-(2,6-dioxopiperidin-3-yl)-1-methyl-1H-indazol-6-yl)piperidin- |
| | 1-yl)methyl)-3-azaspiro[5.5]undecan-3-yl)pyrimidin-4-yl)amino)-1-isopropyl-2-oxo-1,2- |
| | dihydro-1,8-naphthyridin-3-yl)oxy)-N-methylacetamide |
| 238 | 2-[[6-[[5-chloro-2-[4-[[4-[3-(2,6-dioxo-3-piperidyl)-1-methyl-indazol-7-yl]piperazin-1- |
| | yl]methyl]-3-fluoro-1-piperidyl]pyrimidin-4-yl]amino]-1-isopropyl-2-oxo-1,8-naphthyridin- |
| | 3-yl]oxy]-N-methyl-acetamide |
| 238a | rel-2-((6-((5-chloro-2-((3S,4S)-4-((4-(3-(2,6-dioxopiperidin-3-yl)-1-methyl-1H-indazol-7- |
| | yl)piperazin-1-yl)methyl)-3-fluoropiperidin-1-yl)pyrimidin-4-yl)amino)-1-isopropyl-2-oxo- |
| | 1,2-dihydro-1,8-naphthyridin-3-yl)oxy)-N-methylacetamide |
| 238b | rel-2-((6-((5-chloro-2-((3R,4R)-4-((4-(3-(2,6-dioxopiperidin-3-yl)-1-methyl-1H-indazol-7- |
| | yl)piperazin-1-yl)methyl)-3-fluoropiperidin-1-yl)pyrimidin-4-yl)amino)-1-isopropyl-2-oxo- |
| | 1,2-dihydro-1,8-naphthyridin-3-yl)oxy)-N-methylacetamide |
| 239 | 2-((6-((5-chloro-2-(9-((1-(3-(2,6-dioxopiperidin-3-yl)-5-fluoro-1-methyl-1H-indazol-6- |
| | yl)piperidin-4-yl)methyl)-3,9-diazaspiro[5.5]undecan-3-yl)pyrimidin-4-yl)amino)-1- |
| | isopropyl-2-oxo-1,2-dihydro-1,8-naphthyridin-3-yl)oxy)-N-methylacetamide |
| 240 | 2-((6-((5-chloro-2-(9-((4-(3-(2,6-dioxopiperidin-3-yl)-7-fluoro-1-methyl-1H-indazol-6- |
| | yl)piperidin-1-yl)methyl)-3-azaspiro[5.5]undecan-3-yl)pyrimidin-4-yl)amino)-1-isopropyl- |
| | 2-oxo-1,2-dihydro-1,8-naphthyridin-3-yl)oxy)-N-methylacetamide |
| 241 | 2-((6-((5-chloro-2-(4-((1-(3-(2,6-dioxopiperidin-3-yl)-1-methyl-1H-indazol-7-yl)-3,3- |
| | difluoropiperidin-4-yl)methyl)piperazin-1-yl)pyrimidin-4-yl)amino)-1-isopropyl-2-oxo-1,2- |
| | dihydro-1,8-naphthyridin-3-yl)oxy)-N-methylacetamide |
| 242 | 2-[[6-[[5-chloro-2-[4-[[4-[3-(2,6-dioxo-3-piperidyl)-1-methyl-indazol-6-yl]-1- |
| | piperidyl]methyl]-3-fluoro-1-piperidyl]pyrimidin-4-yl]amino]-1-isopropyl-2-oxo-1,8- |
| | naphthyridin-3-yl]oxy]-N-methyl-acetamide |
| 242a | rel-2-((6-((5-chloro-2-((3S,4R)-4-((4-(3-(2,6-dioxopiperidin-3-yl)-1-methyl-1H-indazol-6- |
| | yl)piperidin-1-yl)methyl)-3-fluoropiperidin-1-yl)pyrimidin-4-yl)amino)-1-isopropyl-2-oxo- |
| | 1,2-dihydro-1,8-naphthyridin-3-yl)oxy)-N-methylacetamide |
| 242b | rel-2-((6-((5-chloro-2-((3R,4S)-4-((4-(3-(2,6-dioxopiperidin-3-yl)-1-methyl-1H-indazol-6- |
| | yl)piperidin-1-yl)methyl)-3-fluoropiperidin-1-yl)pyrimidin-4-yl)amino)-1-isopropyl-2-oxo- |
| | 1,2-dihydro-1,8-naphthyridin-3-yl)oxy)-N-methylacetamide |

Certain examples of Formula (I) compounds were synthesized using methods involving resolution of stereoisomeric mixture(s) (e.g., SFC separation of stereoisomers). In Table C1, the resolved stereogenic centers in these compounds are labelled with the “or1” and/or “or2” enhanced stereochemical notations. In some instances, the stereoisomeric resolutions were performed during the last step of the synthesis, thereby providing the individual stereoisomers of the Formula (I) compounds. Alternatively, in some other instances, the resolutions were performed on an intermediate or starting material, wherein each of the constituent stereoisomers of the intermediate or starting material could be separately subjected to the subsequent steps of the synthesis to provide the respective Formula (I) compounds as separate stereoisomers. Methods of resolution and correlation between resolved intermediates and Formula (I) compounds are disclosed in the examples herein and in Table P1. A person of ordinary skill in the art would understand that, under either approach for stereoisomeric resolution, stereoisomers having both (R)- and (S)-configurations at a resolved stereogenic center are provided. See Table C3, wherein Table C1 compounds whose stereoisomers contain the or1 and/or or2 stereochemical notations are provided in non-stereogenic form, followed by the respective stereoisomers having the (R)- and (S)-configurations.

TABLE C3

| No. | Compound Structure |
|---|---|
| 156 | 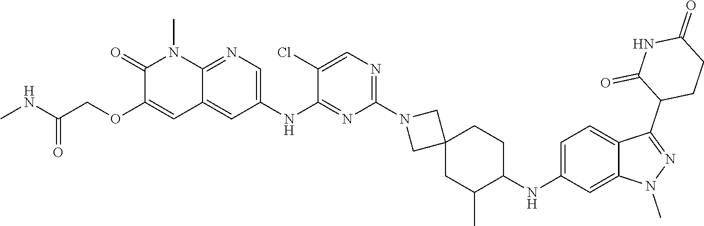 [SMILES: CNC(=O)COc1cc2cc(Nc3nc(N4CC5(CCC(Nc6ccc7c(C8CCC(=O)NC8=O)nn(C)c7c6)C(C)C5)C4)ncc3Cl)cnc2n(C)c1=O] |
| | 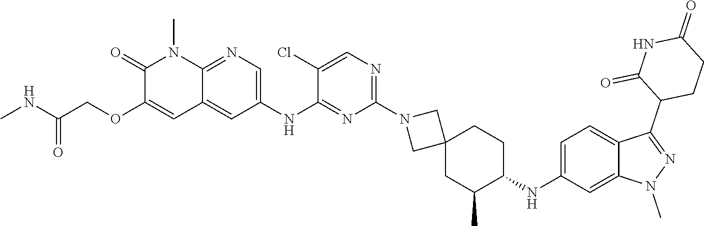 [SMILES: CNC(=O)COc1cc2cc(Nc3nc(N4CC5(CC[C@H](Nc6ccc7c(C8CCC(=O)NC8=O)nn(C)c7c6)[C@@H](C)C5)C4)ncc3Cl)cnc2n(C)c1=O] |
| | 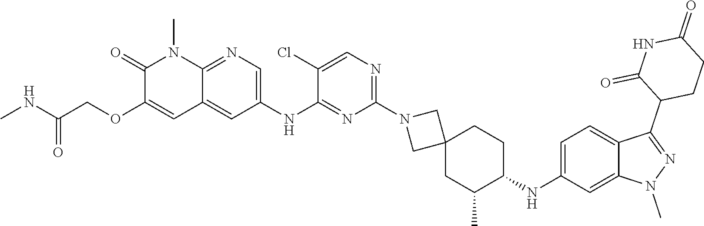 [SMILES: CNC(=O)COc1cc2cc(Nc3nc(N4CC5(CC[C@H](Nc6ccc7c(C8CCC(=O)NC8=O)nn(C)c7c6)[C@H](C)C5)C4)ncc3Cl)cnc2n(C)c1=O] |
| | 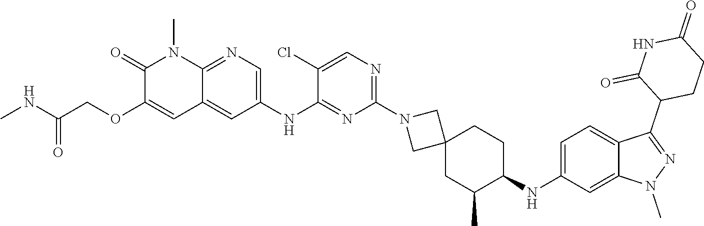 [SMILES: CNC(=O)COc1cc2cc(Nc3nc(N4CC5(CC[C@@H](Nc6ccc7c(C8CCC(=O)NC8=O)nn(C)c7c6)[C@@H](C)C5)C4)ncc3Cl)cnc2n(C)c1=O] |
| | 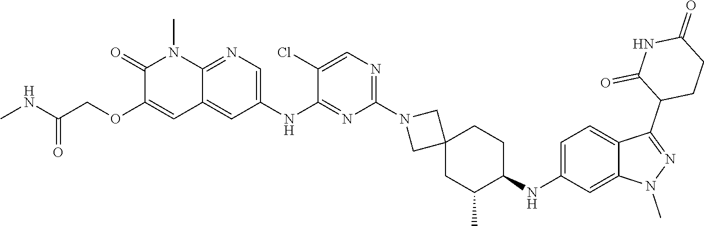 [SMILES: CNC(=O)COc1cc2cc(Nc3nc(N4CC5(CC[C@@H](Nc6ccc7c(C8CCC(=O)NC8=O)nn(C)c7c6)[C@H](C)C5)C4)ncc3Cl)cnc2n(C)c1=O] |
| 159 | 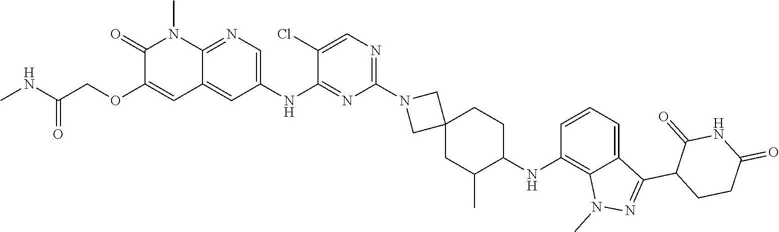 [SMILES: CNC(=O)COc1cc2cc(Nc3nc(N4CC5(CCC(Nc6cccc7c(C8CCC(=O)NC8=O)nn(C)c67)C(C)C5)C4)ncc3Cl)cnc2n(C)c1=O] |
| | 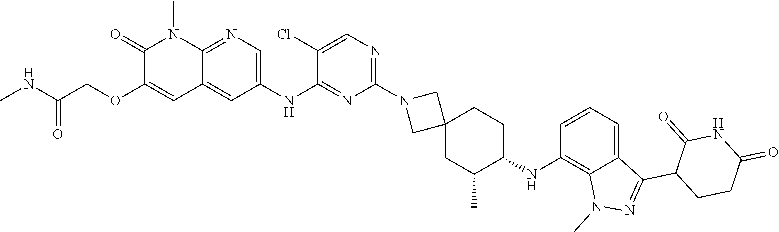 [SMILES: CNC(=O)COc1cc2cc(Nc3nc(N4CC5(CC[C@H](Nc6cccc7c(C8CCC(=O)NC8=O)nn(C)c67)[C@H](C)C5)C4)ncc3Cl)cnc2n(C)c1=O] |
| | 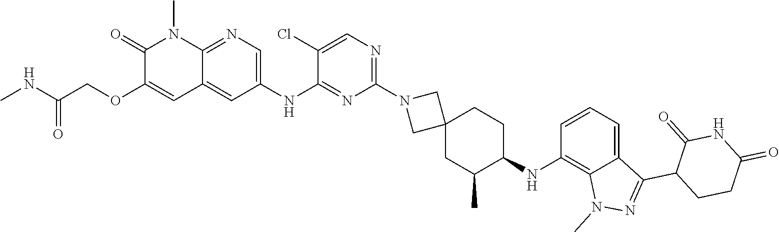 [SMILES: CNC(=O)COc1cc2cc(Nc3nc(N4CC5(CC[C@@H](Nc6cccc7c(C8CCC(=O)NC8=O)nn(C)c67)[C@@H](C)C5)C4)ncc3Cl)cnc2n(C)c1=O] |
| | 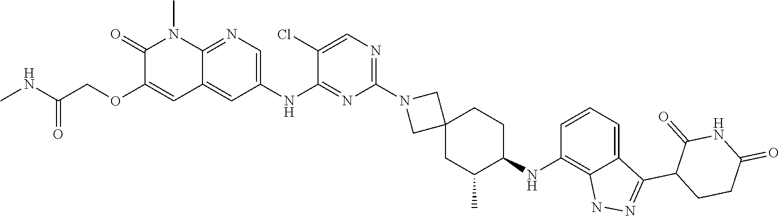 [SMILES: CNC(=O)COc1cc2cc(Nc3nc(N4CC5(CC[C@@H](Nc6cccc7c(C8CCC(=O)NC8=O)n[nH]c67)[C@H](C)C5)C4)ncc3Cl)cnc2n(C)c1=O] |
| | 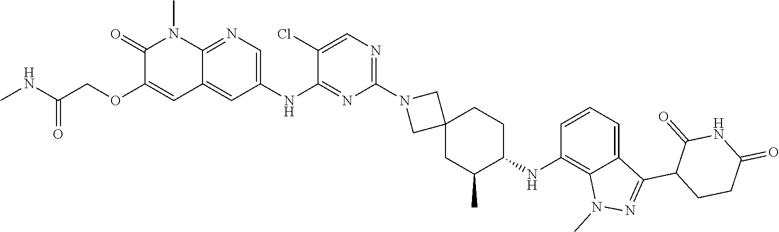 [SMILES: CNC(=O)COc1cc2cc(Nc3nc(N4CC5(CC[C@H](Nc6cccc7c(C8CCC(=O)NC8=O)nn(C)c67)[C@@H](C)C5)C4)ncc3Cl)cnc2n(C)c1=O] |
| 180 | 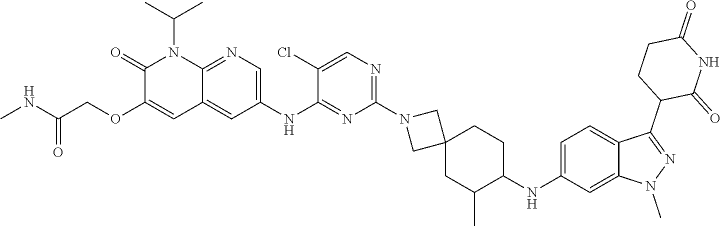 [SMILES: CNC(=O)COc1cc2cc(Nc3nc(N4CC5(CCC(Nc6ccc7c(C8CCC(=O)NC8=O)nn(C)c7c6)C(C)C5)C4)ncc3Cl)cnc2n(C(C)C)c1=O] |
| | 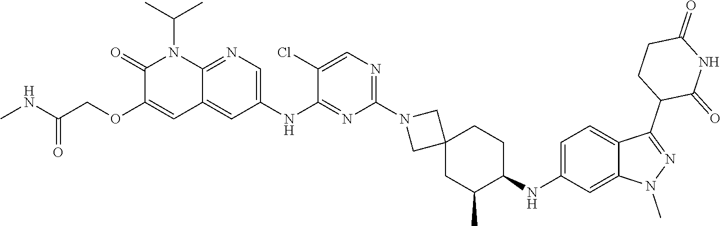 [SMILES: CNC(=O)COc1cc2cc(Nc3nc(N4CC5(CC[C@@H](Nc6ccc7c(C8CCC(=O)NC8=O)nn(C)c7c6)[C@@H](C)C5)C4)ncc3Cl)cnc2n(C(C)C)c1=O] |
| | 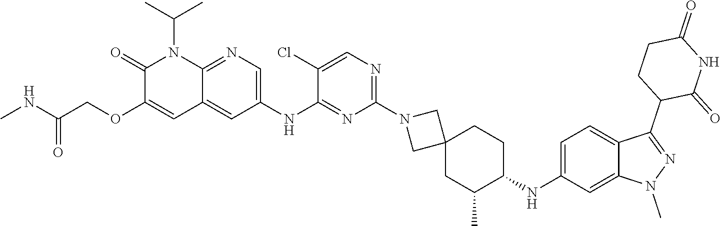 [SMILES: CNC(=O)COc1cc2cc(Nc3nc(N4CC5(CC[C@H](Nc6ccc7c(C8CCC(=O)NC8=O)nn(C)c7c6)[C@H](C)C5)C4)ncc3Cl)cnc2n(C(C)C)c1=O] |
| | 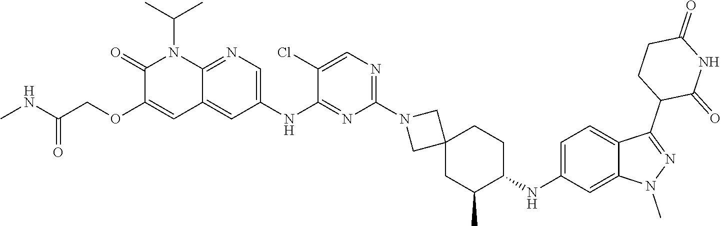 [SMILES: CNC(=O)COc1cc2cc(Nc3nc(N4CC5(CC[C@H](Nc6ccc7c(C8CCC(=O)NC8=O)nn(C)c7c6)[C@@H](C)C5)C4)ncc3Cl)cnc2n(C(C)C)c1=O] |
| | 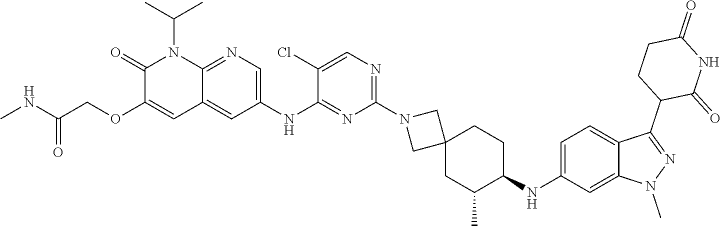 [SMILES: CNC(=O)COc1cc2cc(Nc3nc(N4CC5(CC[C@@H](Nc6ccc7c(C8CCC(=O)NC8=O)nn(C)c7c6)[C@H](C)C5)C4)ncc3Cl)cnc2n(C(C)C)c1=O] |
| 185 | 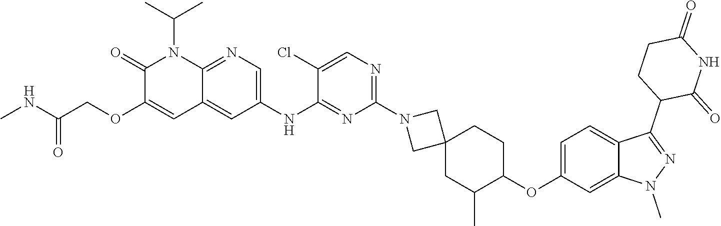 [SMILES: CNC(=O)COc1cc2cc(Nc3nc(N4CC5(CCC(Oc6ccc7c(C8CCC(=O)NC8=O)nn(C)c7c6)C(C)C5)C4)ncc3Cl)cnc2n(C(C)C)c1=O] |
| | 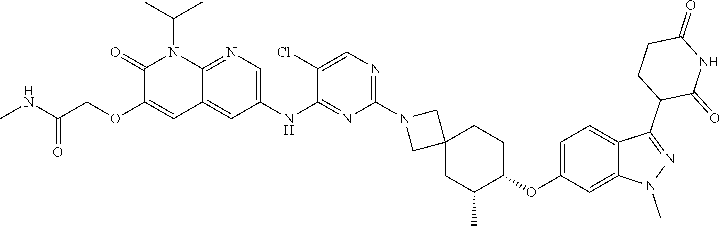 [SMILES: CNC(=O)COc1cc2cc(Nc3nc(N4CC5(CC[C@H](Oc6ccc7c(C8CCC(=O)NC8=O)nn(C)c7c6)[C@H](C)C5)C4)ncc3Cl)cnc2n(C(C)C)c1=O] |
| | 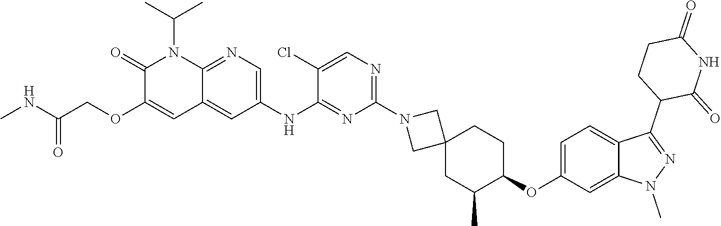 [SMILES: CNC(=O)COc1cc2cc(Nc3nc(N4CC5(CC[C@@H](Oc6ccc7c(C8CCC(=O)NC8=O)nn(C)c7c6)[C@@H](C)C5)C4)ncc3Cl)cnc2n(C(C)C)c1=O] |
| | 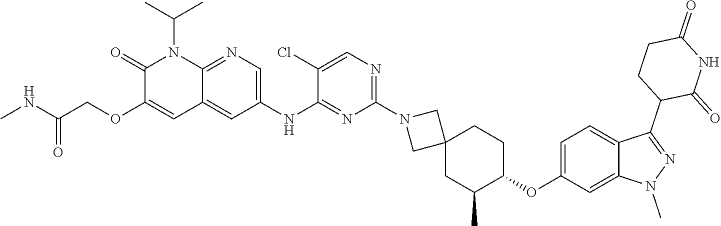 [SMILES: CNC(=O)COc1cc2cc(Nc3nc(N4CC5(CC[C@H](Oc6ccc7c(C8CCC(=O)NC8=O)nn(C)c7c6)[C@@H](C)C5)C4)ncc3Cl)cnc2n(C(C)C)c1=O] |
| | 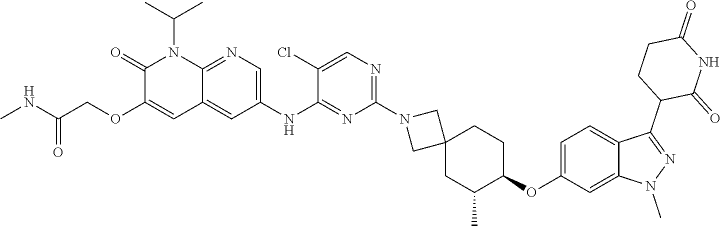 [SMILES: CNC(=O)COc1cc2cc(Nc3nc(N4CC5(CC[C@@H](Oc6ccc7c(C8CCC(=O)NC8=O)nn(C)c7c6)[C@H](C)C5)C4)ncc3Cl)cnc2n(C(C)C)c1=O] |
| 193 | 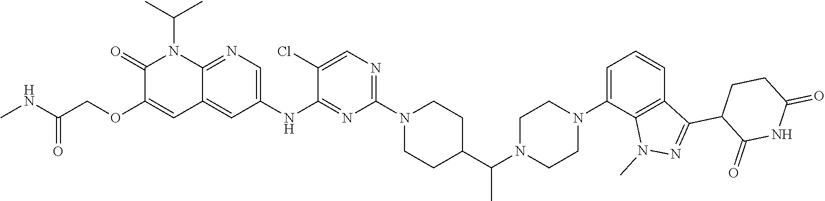 [SMILES: CNC(=O)COc1cc2cc(Nc3nc(N4CCC(C(C)N5CCN(c6cccc7c(C8CCC(=O)NC8=O)nn(C)c67)CC5)CC4)ncc3Cl)cnc2n(C(C)C)c1=O] |
| | 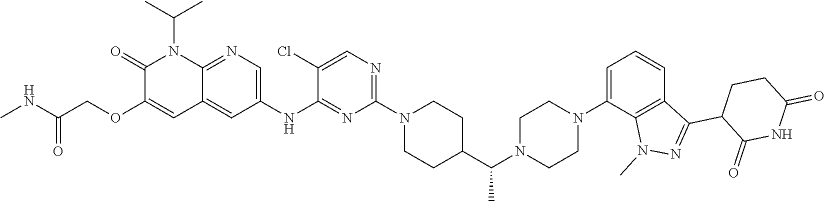 [SMILES: CNC(=O)COc1cc2cc(Nc3nc(N4CCC([C@@H](C)N5CCN(c6cccc7c(C8CCC(=O)NC8=O)nn(C)c67)CC5)CC4)ncc3Cl)cnc2n(C(C)C)c1=O] |
| | 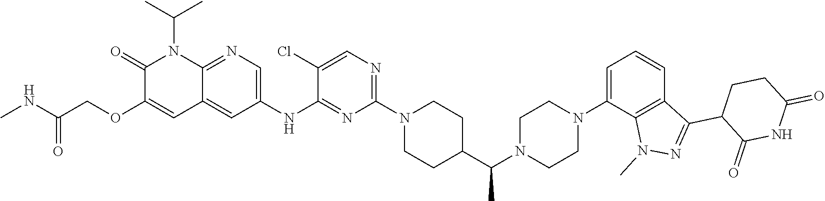 [SMILES: CNC(=O)COc1cc2cc(Nc3nc(N4CCC([C@H](C)N5CCN(c6cccc7c(C8CCC(=O)NC8=O)nn(C)c67)CC5)CC4)ncc3Cl)cnc2n(C(C)C)c1=O] |
| 194 | 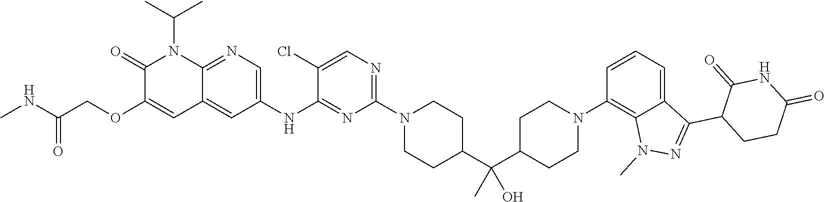 [SMILES: CNC(=O)COc1cc2cc(Nc3nc(N4CCC(C(C)(O)C5CCN(c6cccc7c(C8CCC(=O)NC8=O)nn(C)c67)CC5)CC4)ncc3Cl)cnc2n(C(C)C)c1=O] |
| | 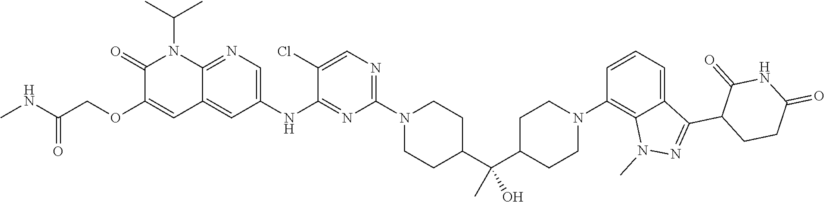 [SMILES: CNC(=O)COc1cc2cc(Nc3nc(N4CCC([C@@](C)(O)C5CCN(c6cccc7c(C8CCC(=O)NC8=O)nn(C)c67)CC5)CC4)ncc3Cl)cnc2n(C(C)C)c1=O] |
| | 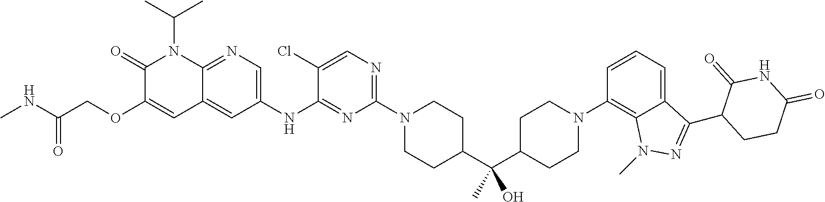 [SMILES: CNC(=O)COc1cc2cc(Nc3nc(N4CCC([C@](C)(O)C5CCN(c6cccc7c(C8CCC(=O)NC8=O)nn(C)c67)CC5)CC4)ncc3Cl)cnc2n(C(C)C)c1=O] |
| 195 | 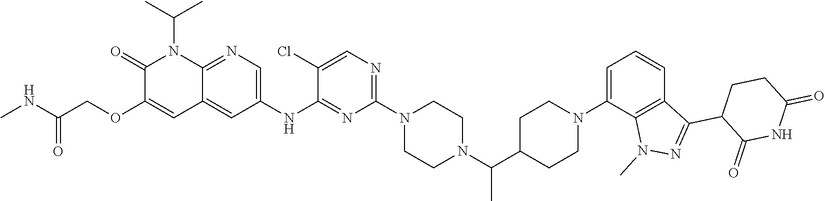 [SMILES: CNC(=O)COc1cc2cc(Nc3nc(N4CCN(C(C)C5CCN(c6cccc7c(C8CCC(=O)NC8=O)nn(C)c67)CC5)CC4)ncc3Cl)cnc2n(C(C)C)c1=O] |
| | 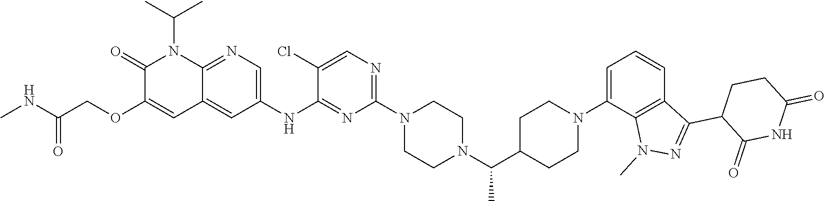 [SMILES: CNC(=O)COc1cc2cc(Nc3nc(N4CCN([C@@H](C)C5CCN(c6cccc7c(C8CCC(=O)NC8=O)nn(C)c67)CC5)CC4)ncc3Cl)cnc2n(C(C)C)c1=O] |
| | 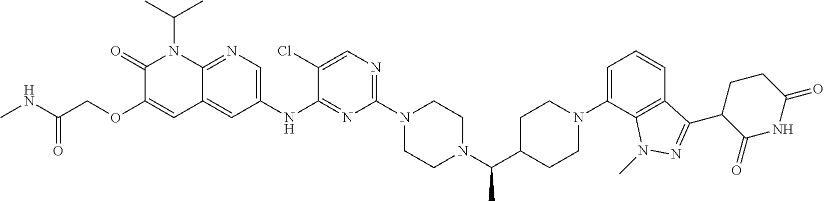 [SMILES: CNC(=O)COc1cc2cc(Nc3nc(N4CCN([C@H](C)C5CCN(c6cccc7c(C8CCC(=O)NC8=O)nn(C)c67)CC5)CC4)ncc3Cl)cnc2n(C(C)C)c1=O] |
| 201 | 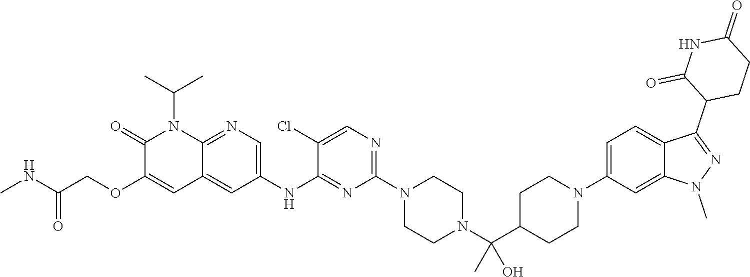 [SMILES: CNC(=O)COc1cc2cc(Nc3nc(N4CCN(C(C)(O)C5CCN(c6ccc7c(C8CCC(=O)NC8=O)nn(C)c7c6)CC5)CC4)ncc3Cl)cnc2n(C(C)C)c1=O] |
| | 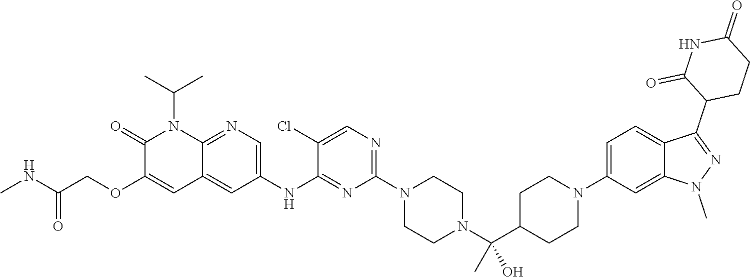 [SMILES: CNC(=O)COc1cc2cc(Nc3nc(N4CCN([C@@](C)(O)C5CCN(c6ccc7c(C8CCC(=O)NC8=O)nn(C)c7c6)CC5)CC4)ncc3Cl)cnc2n(C(C)C)c1=O] |
| | 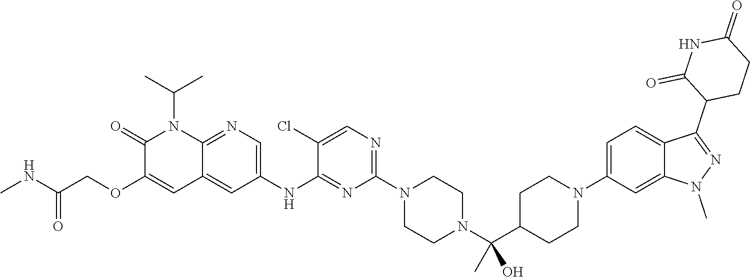 [SMILES: CNC(=O)COc1cc2cc(Nc3nc(N4CCN([C@](C)(O)C5CCN(c6ccc7c(C8CCC(=O)NC8=O)nn(C)c7c6)CC5)CC4)ncc3Cl)cnc2n(C(C)C)c1=O] |
| 221 | 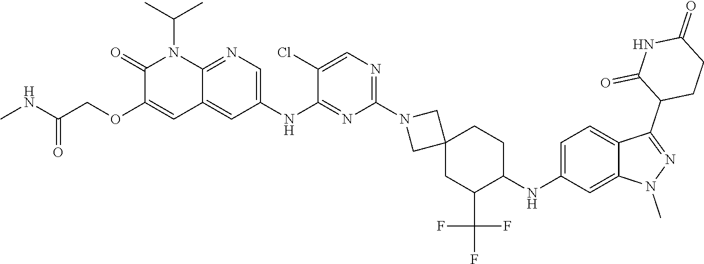 [SMILES: CNC(=O)COc1cc2cc(Nc3nc(N4CC5(CCC(Nc6ccc7c(C8CCC(=O)NC8=O)nn(C)c7c6)C(C(F)(F)F)C5)C4)ncc3Cl)cnc2n(C(C)C)c1=O] |
| | 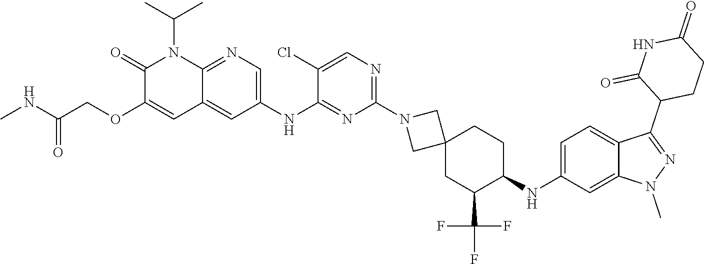 [SMILES: CNC(=O)COc1cc2cc(Nc3nc(N4CC5(CC[C@@H](Nc6ccc7c(C8CCC(=O)NC8=O)nn(C)c7c6)[C@@H](C(F)(F)F)C5)C4)ncc3Cl)cnc2n(C(C)C)c1=O] |
| | 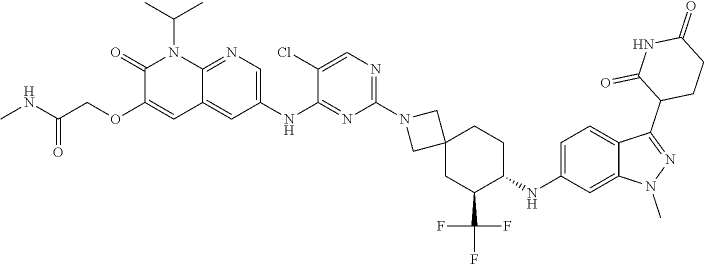 [SMILES: CNC(=O)COc1cc2cc(Nc3nc(N4CC5(CC[C@H](Nc6ccc7c(C8CCC(=O)NC8=O)nn(C)c7c6)[C@@H](C(F)(F)F)C5)C4)ncc3Cl)cnc2n(C(C)C)c1=O] |
| | 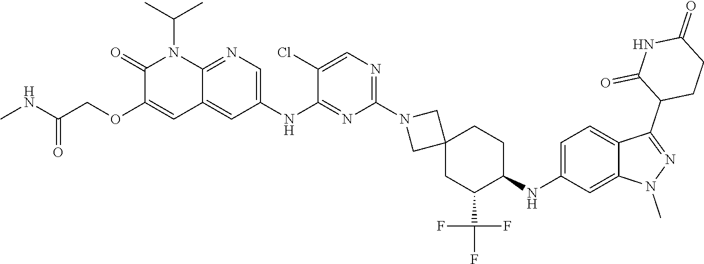 [SMILES: CNC(=O)COc1cc2cc(Nc3nc(N4CC5(CC[C@@H](Nc6ccc7c(C8CCC(=O)NC8=O)nn(C)c7c6)[C@H](C(F)(F)F)C5)C4)ncc3Cl)cnc2n(C(C)C)c1=O] |
| | 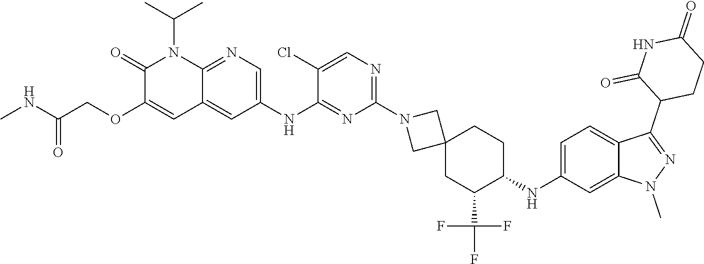 [SMILES: CNC(=O)COc1cc2cc(Nc3nc(N4CC5(CC[C@H](Nc6ccc7c(C8CCC(=O)NC8=O)nn(C)c7c6)[C@H](C(F)(F)F)C5)C4)ncc3Cl)cnc2n(C(C)C)c1=O] |
| 238 | 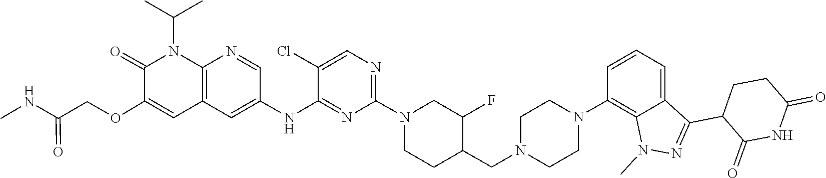 [SMILES: CNC(=O)COc1cc2cc(Nc3nc(N4CCC(CN5CCN(c6cccc7c(C8CCC(=O)NC8=O)nn(C)c67)CC5)C(F)C4)ncc3Cl)cnc2n(C(C)C)c1=O] |
| | 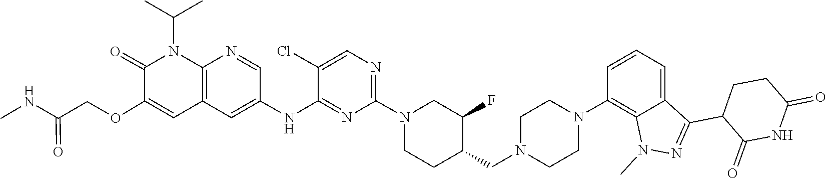 [SMILES: CNC(=O)COc1cc2cc(Nc3nc(N4CC[C@@H](CN5CCN(c6cccc7c(C8CCC(=O)NC8=O)nn(C)c67)CC5)[C@H](F)C4)ncc3Cl)cnc2n(C(C)C)c1=O] |
| | 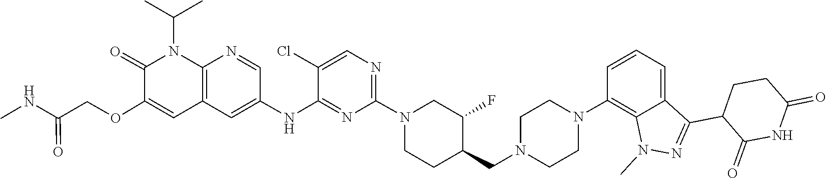 [SMILES: CNC(=O)COc1cc2cc(Nc3nc(N4CC[C@H](CN5CCN(c6cccc7c(C8CCC(=O)NC8=O)nn(C)c67)CC5)[C@@H](F)C4)ncc3Cl)cnc2n(C(C)C)c1=O] |
| 242 | 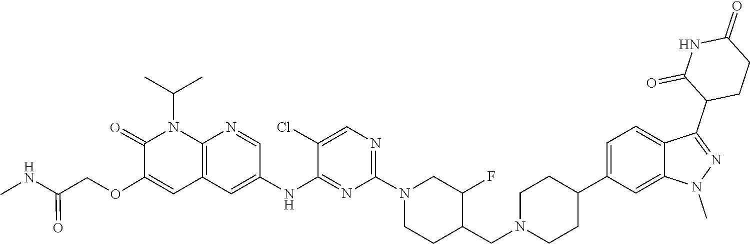 [SMILES: CNC(=O)COc1cc2cc(Nc3nc(N4CCC(CN5CCC(c6ccc7c(C8CCC(=O)NC8=O)nn(C)c7c6)CC5)C(F)C4)ncc3Cl)cnc2n(C(C)C)c1=O] |
| | 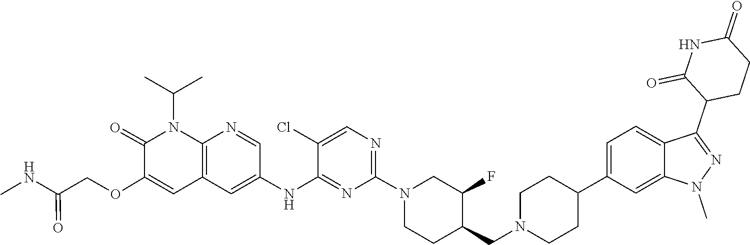 [SMILES: CNC(=O)COc1cc2cc(Nc3nc(N4CC[C@H](CN5CCC(c6ccc7c(C8CCC(=O)NC8=O)nn(C)c7c6)CC5)[C@H](F)C4)ncc3Cl)cnc2n(C(C)C)c1=O] |
| | 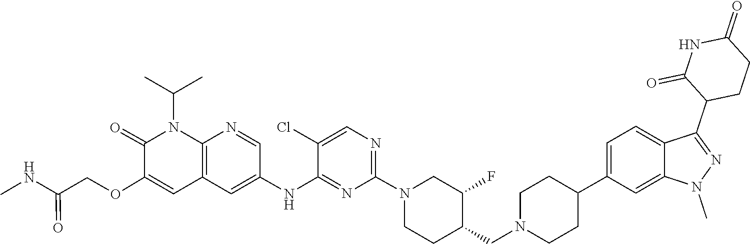 [SMILES: CNC(=O)COc1cc2cc(Nc3nc(N4CC[C@@H](CN5CCC(c6ccc7c(C8CCC(=O)NC8=O)nn(C)c7c6)CC5)[C@@H](F)C4)ncc3Cl)cnc2n(C(C)C)c1=O] |

Exemplary compounds of Formula (I) also include those depicted in Table C1 of U.S. Provisional Application Ser. No. 63/407,006, filed Sep. 15, 2022; Table C1 of U.S. Provisional Application Ser. No. 63/407,012, filed Sep. 15, 2022; Table C1 of U.S. Provisional Application Ser. No. 63/436,009, filed Dec. 29, 2022; Table C1 of U.S. Provisional Application Ser. No. 63/497,054, filed Apr. 19, 2023; and Table C1 of U.S. Provisional Application Ser. No. 63/501,077, filed May 9, 2023; or pharmaceutically acceptable salts thereof, wherein each Table C1 is incorporated herein by reference in its entirety.

In some embodiments, the compounds of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or pharmaceutically acceptable salts thereof, reduce cell viability in a cell line expressing a BCL6 protein with an EC₅₀ of less than 1 μM (e.g., less than 750 nM, less than 500 nM, or less than 200 nM). In some embodiments, the compounds reduce cell viability in a cell line expressing the BCL6 protein with an EC50 of less than 200 nM (e.g., less than 150 nM, less than 200 nM, less than 100 nM, less than 10 nM, less than 1 nM). For example, the compounds can reduce cell viability in a cell line expressing the BCL6 protein with an EC50 of about 0.1 nM to about 100 nM, about 0.1 nM to about 50 nM, about 1 nM to about 50 nM, about 1 nM to about 20 nM, or about 0.1 nM to about 1 nM.

In some embodiments, the compounds of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or pharmaceutically acceptable salts thereof, induce degradation of a BCL6 protein in a cell line expressing the BCL6 protein with a DC₅₀ of less than 1 μM (e.g., less than 750 nM, less than 500 nM, or less than 200 nM). In some embodiments, the compounds of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or pharmaceutically acceptable salts thereof, induce degradation of a BCL6 protein in a cell line expressing the BCL6 protein with a DC50 of less than 200 nM (e.g., less than 150 nM, less than 200 nM, less than 100 nM, less than 10 nM, less than 1 nM). For example, the compounds can induce degradation of a BCL6 protein in a cell line expressing the BCL6 protein with a DC50 of about 0.1 nM to about 100 nM, about 0.1 nM to about 50 nM, about 1 nM to about 50 nM, about 1 nM to about 20 nM, or about 0.1 nM to about 1 nM.

In some embodiments, the compounds of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or pharmaceutically acceptable salts thereof, induce degradation of a BCL6 protein in a cell line expressing the BCL6 protein with a Y min of less than 70% (e.g., less than 50%, less than 30%, less than 20%, or less than 10%). In some embodiments, the compounds of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or pharmaceutically acceptable salts thereof, induce degradation of a BCL6 protein in a cell line expressing the BCL6 protein with a Yₘᵢₙ of less than 50% (e.g., less than 40%, less than 30%, less than 20%, less than 10%, or less than 5%). In some embodiments, the compounds of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or pharmaceutically acceptable salts thereof, induce degradation of a BCL6 protein in a cell line expressing the BCL6 protein with a Yₘᵢₙ of less than 30% (e.g., less than 25%, less than 20%, less than 15%, less than 10%, or less than 5%). For example, the compounds can induce degradation of a BCL6 protein in a cell line expressing the BCL6 protein with a Yₘᵢₙ of about 1% to about 70% (e.g., about 5% to about 50% or about 10% to about 30%).

Also provided herein is a BCL6 protein non-covalently bound with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof.

Also provided herein is a ternary complex comprising a BCL6 protein, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, and a CRBN protein or a portion thereof.

### Chemical Definitions

The term “halo” refers to fluoro (F), chloro (Cl), bromo (Br), or iodo (I).

The term “oxo” refers to a divalent doubly bonded oxygen atom (i.e., “═O”). As used herein, oxo groups are attached to carbon atoms to form carbonyls.

The term “alkyl” refers to a saturated acyclic hydrocarbon radical that may be a straight chain or branched chain, containing the indicated number of carbon atoms. For example, C1-10 indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo (e.g., —CF₃, —CHF₂, or —CH₂F).

The term “alkoxy” refers to an —O-alkyl radical (e.g., —OCH₃).

The term “alkylene” refers to a divalent alkyl (e.g., —CH₂—). Similarly, terms such as “cycloalkylene” and “heterocyclylene” refer to divalent cycloalkyl and heterocyclyl respectively. For avoidance of doubt, in “cycloalkylene” and “heterocyclylene”, the two radicals can be on the same ring carbon atom (e.g., a geminal diradical such as

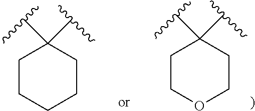 [SMILES: *C1(*)CCCCC1 ; *C1(*)CCOCC1]

or on different ring atoms (e.g., ring carbon and/or nitrogen atoms (e.g., vicinal ring carbon and/or nitrogen atoms))

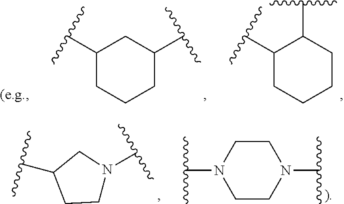 [SMILES: *C1CCCC(*)C1 ; *C1CCCCC1* ; *C1CCN(*)C1 ; *N1CCN(*)CC1]

The term “alkenyl” refers to an acyclic hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it. Alkenyl groups can either be unsubstituted or substituted with one or more substituents.

The term “alkynyl” refers to an acyclic hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it. Alkynyl groups can either be unsubstituted or substituted with one or more substituents.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein refers to mono-, bi-, tri-, or polycyclic saturated or partially unsaturated hydrocarbon groups having, e.g., 3 to 20 ring carbons, preferably 3 to 15 ring carbons, and more preferably 3 to 12 ring carbons or 3-10 ring carbons or 3-6 ring carbons, wherein the cycloalkyl group may be optionally substituted. The term “saturated” as used in this context means only single bonds present between constituent carbon atoms. Examples of saturated cycloalkyl groups include, without limitation, cyclopropyl, cyclobutyl, cyclopentyl, cyclohexyl, cycloheptyl, and cyclooctyl. Partially unsaturated cycloalkyl may have any degree of unsaturation provided that one or more double bonds is present in the cycloalkyl, none of the rings in the ring system are aromatic, and the partially unsaturated cycloalkyl group is not fully saturated overall. Examples of partially unsaturated cycloalkyl include, without limitation, cyclopentenyl, cyclohexenyl, cycloheptenyl, and cyclooctenyl. Cycloalkyl may include multiple fused and/or bridged rings. Non-limiting examples of fused/bridged cycloalkyl includes: bicyclo[1.1.0]butyl, bicyclo[2.1.0]pentyl, bicyclo[1.1.1]pentyl, bicyclo[3.1.0]hexyl, bicyclo[2.1.1]hexyl, bicyclo[3.2.0]heptyl, bicyclo[4.1.0]heptyl, bicyclo[2.2.1]heptyl, bicyclo[3.1.1]heptyl, bicyclo[4.2.0]octyl, bicyclo[3.2.1]octyl, bicyclo[2.2.2]octyl, and the like. Cycloalkyl also includes spirocyclic rings (e.g., spirocyclic bicycle wherein two rings are connected through just one atom). Non-limiting examples of spirocyclic cycloalkyls include spiro[2.2]pentyl, spiro[2.5]octyl, spiro[3.5]nonyl, spiro[3.5]nonyl, spiro[3.5]nonyl, spiro[4.4]nonyl, spiro[2.6]nonyl, spiro[4.5]decyl, spiro[3.6]decyl, spiro[5.5]undecyl, and the like.

The term “heteroaryl”, as used herein, means a mono-, bi-, tri- or polycyclic group having 5 to 20 ring atoms, alternatively 5, 6, 9, 10, or 15 ring atoms; wherein at least one ring in the system contains one or more heteroatoms independently selected from the group consisting of N, O, and S (inclusive of oxidized forms such as:

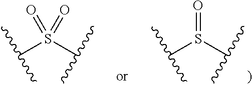

and at least one ring in the system is aromatic (but does not have to be a ring which contains a heteroatom, e.g. tetrahydroisoquinolinyl, e.g., tetrahydroquinolinyl). In some embodiments, heteroaryl groups contain 1-4 (e.g., 1, 2, or 3) ring heteroatoms each independently selected from the group consisting of N, O, and S (inclusive of oxidized forms such as:

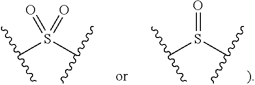

Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridinyl, pyrazolo[4,3-b]pyridinyl, tetrazolyl, chromanyl, 2,3-dihydrobenzo[b][1,4]dioxinyl, benzo[d][1,3]dioxolyl, 2,3-dihydrobenzofuranyl, tetrahydroquinolinyl, 2,3-dihydrobenzo[b][1,4]oxathiinyl, isoindolinyl, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

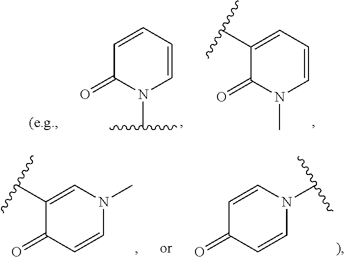

pyrimidone

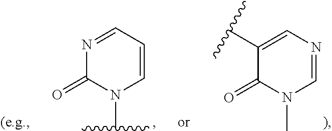

pyridazinone

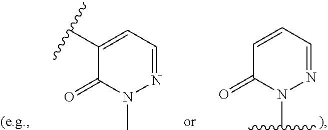

pyrazinone

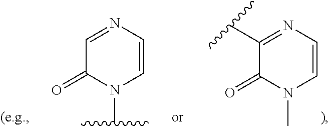

and imidazolone

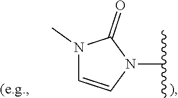 [SMILES: *n1ccn(C)c1=O]

wherein each ring nitrogen adjacent to a carbonyl is tertiary (i.e., the oxo group (i.e., “═O”) herein is a constituent part of the heteroaryl ring).

The term “heterocyclyl” refers to a mono-, bi-, tri-, or polycyclic saturated or partially unsaturated ring system with 3-15 ring atoms (e.g., 5-8 membered monocyclic, 8-12 membered bicyclic, or 11-15 membered tricyclic ring system) having 1-3 heteroatoms if monocyclic, 1-6 heteroatoms if bicyclic, or 1-9 heteroatoms if tricyclic or polycyclic, said heteroatoms selected from O, N, and S (inclusive of oxidized forms such as:

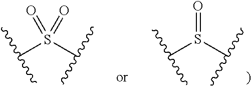

(e.g., carbon atoms and 1-3, 1-6, or 1-9 heteroatoms of N, O, S, or P if monocyclic, bicyclic, or tricyclic, respectively), wherein 0, 1, 2 or 3 atoms of each ring may be substituted by a substituent. The term “saturated” as used in this context means only single bonds present between constituent ring atoms and other available valences occupied by hydrogen and/or other substituents as defined herein. Examples of saturated heterocyclyl groups include piperazinyl, pyrrolidinyl, dioxanyl, morpholinyl, tetrahydrofuranyl, and the like. Partially unsaturated heterocyclyl groups may have any degree of unsaturation provided that one or more double bonds is present in the heterocyclyl, none of the rings in the ring system are aromatic, and the partially unsaturated heterocyclyl group is not fully saturated overall. Examples of partially unsaturated heterocyclyl groups include, without limitation, tetrahydropyridyl, dihydropyrazinyl, dihydropyridyl, dihydropyrrolyl, dihydrofuranyl, dihydrothiophenyl.

Heterocyclyl may include multiple fused and bridged rings. Non-limiting examples of fused/bridged heteorocyclyl includes: 2-azabicyclo[1.1.0]butyl, 2-azabicyclo[2.1.0]pentyl, 2-azabicyclo[1.1.1]pentyl, 3-azabicyclo[3.1.0]hexyl, 5-azabicyclo[2.1.1]hexyl, 3-azabicyclo[3.2.0]heptyl, octahydrocyclopenta[c]pyrrolyl, 3-azabicyclo[4.1.0]heptyl, 7-azabicyclo[2.2.1]heptyl, 6-azabicyclo[3.1.1]heptyl, 7-azabicyclo[4.2.0]octyl, 2-azabicyclo[2.2.2]octyl, 3-azabicyclo[3.2.1]octyl, 2-oxabicyclo[1.1.0]butyl, 2-oxabicyclo[2.1.0]pentyl, 2-oxabicyclo[1.1.1]pentyl, 3-oxabicyclo[3.1.0]hexyl, 5-oxabicyclo[2.1.1]hexyl, 3-oxabicyclo[3.2.0]heptyl, 3-oxabicyclo[4.1.0]heptyl, 7-oxabicyclo[2.2.1]heptyl, 6-oxabicyclo[3.1.1]heptyl, 7-oxabicyclo[4.2.0]octyl, 2-oxabicyclo[2.2.2]octyl, 3-oxabicyclo[3.2.1]octyl, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentyl, 4-azaspiro[2.5]octyl, 1-azaspiro[3.5]nonyl, 2-azaspiro[3.5]nonyl, 7-azaspiro[3.5]nonyl, 2-azaspiro[4.4]nonyl, 6-azaspiro[2.6]nonyl, 1,7-diazaspiro[4.5]decyl, 7-azaspiro[4.5]decyl 2,5-diazaspiro[3.6]decyl, 3-azaspiro[5.5]undecyl, 2-oxaspiro[2.2]pentyl, 4-oxaspiro[2.5]octyl, 1-oxaspiro[3.5]nonyl, 2-oxaspiro[3.5]nonyl, 7-oxaspiro[3.5]nonyl, 2-oxaspiro[4.4]nonyl, 6-oxaspiro[2.6]nonyl, 1,7-dioxaspiro[4.5]decyl, 2,5-dioxaspiro[3.6]decyl, 1-oxaspiro[5.5]undecyl, 3-oxaspiro[5.5]undecyl, 3-oxa-9-azaspiro[5.5]undecyl and the like.

A nitrogen-containing heterocyclyl as used herein refers to a heterocyclyl having 1-2 ring nitrogen atoms and 0-2 additional ring heteroatoms selected from the group consisting of O and S (inclusive of oxidized such as:

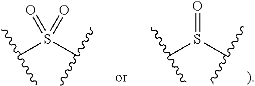

The nitrogen-containing heterocyclyl can be monocyclic, bicyclic, or polycyclic as defined elsewhere herein. Examples of monocyclic nitrogen-containing heterocyclyl include azetidinyl, pyrrolidinyl, piperidinyl, piperazinyl, morpholinyl, and the like. Examples of bicyclic nitrogen-containing heterocyclyl include 7-azaspiro[3.5]nonyl, 1,7-diazaspiro[4.5]decyl, 3-oxa-7,9-diazabicyclo[3.3.1]nonanyl, 2,6-diazaspiro[3.3]heptanyl, and the like.

As used herein, when a ring is described as being “partially unsaturated”, it means said ring has one or more additional degrees of unsaturation (in addition to the degree of unsaturation attributed to the ring itself; e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, heterocycloalkenyl, cycloalkenyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

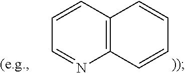

(ii) a single ring atom (spiro-fused ring systems)

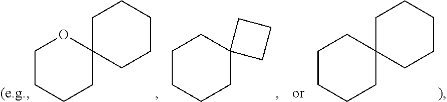

or (iii) a contiguous array of ring atoms (bridged ring systems having all bridge lengths >0)

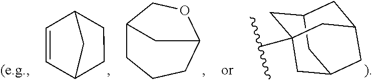

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

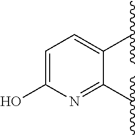 [SMILES: *c1ccc(O)nc1*]

encompasses the tautomeric form containing the moiety:

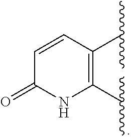 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Unless otherwise indicated, when a disclosed compound is named or depicted by a structure without specifying the stereochemistry and has one or more chiral centers, it is understood to represent all possible stereoisomers of the compound.

### Methods of Treatment

### Indications

Provided herein are methods for inducing degradation of a BCL6 protein. For example, provided herein are compounds capable of inducing degradation of a BCL6 protein useful for treating or preventing cancers. Exemplary compounds that bind to BCL6 are described in, e.g., Cerchietti, Leandro C., et al. Cancer Cell 17.4 (2010): 400-411; Cardenas, Mariano G., et al. The Journal of Clinical Investigation 126(9) (2016): 3351-3362; Kerres, Nina, et al., Cell Reports 20.12 (2017): 2860-2875; Yasui, Takeshi, et al., Bioorganic & Medicinal Chemistry 25.17 (2017): 4876-4886; Kamada, Yusuke, et al., Journal of Medicinal Chemistry 60.10 (2017): 4358-4368; McCoull, William, et al., ACS Chemical Biology 13.11 (2018): 3131-3141; Guo, Weikai, et al. Journal of Medicinal Chemistry 63.2 (2020): 676-695; Teng, Mingxing, et al., ACS Medicinal Chemistry Letters 11.6 (2020): 1269-1273; Pearce, Andrew C., et al., Journal of Biological Chemistry 297.2 (2021); Ding, Shu, Yu Rao, and Qianjin Lu, Cellular & Molecular Immunology (2022): 1-3; Xing, Y. et al., Cancer Letters (2022), doi: 10.1016/j.canlet.2021.12.035; Huckvale, R. et al., Journal of Medicinal Chemistry (2022), doi: 10.1021/acs.jmedchem. 1c02175; Davis, O. et al., Journal of Medicinal Chemistry (2022), doi: 10.1021/acs.jmedchem.1c02174; International Publication Nos. WO 2008/066887; WO 2010/008436; WO 2014/204859; WO 2018/215798; WO 2018/215801; WO 2018/219281; WO 2019/119138; WO 2019/119144; WO 2019/119145; WO 2019/153080; WO 2019/197842; WO 2020/104820; WO 2021/074620; WO 2021/077010, and WO 2022/221673.

Potency of degradation by a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, as provided herein can be determined by DC₅₀ value. As used herein, DC₅₀ refers to the concentration of the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))) that results in a 50% decrease in the concentration of a protein (e.g., BCL6 protein) in a cell compared to the concentration of the protein before the cell is contacted with the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or compared to the concentration of the protein in a cell not contacted with the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))). A compound with a lower DC₅₀ value, as determined under substantially similar conditions, is a more potent inducer of degradation relative to a compound with a higher DC₅₀ value. In some embodiments, a DC₅₀ value can be determined (e.g., using HiBiT detection) in vitro or in vivo (e.g., in tumor cells (e.g., cell lines such as A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RI1, R^{L}, SU-DHL-4, SU-DHL-5, SU-DHL-6, SU-DHL-8, SU-DHL-10, VAL, or WSU-DLCL2; see also those disclosed in, e.g., Cardenas, Mariano G., et al. Clinical Cancer Research 23.4 (2017): 885-893 and International Publication Nos. WO 2021/080950, WO 2021/077010, and WO 2022/221673) expressing a BCL6 protein)). In some embodiments, a cell line that is not dependent on BCL6 and/or that does not have significant expression of BCL6 can be used as a control (e.g., Toledo, H929, MM.1S, or OPM2).

Potency of degradation by a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, as provided herein can be determined by EC₅₀ value. As used herein, EC₅₀ refers to the concentration of the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))) that results in a 50% decrease in the concentration of a protein (e.g., BCL6 protein) relative to the trough concentration of the protein in a cell, when compared to the concentration of the protein before the cell is contacted with the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or compared to the concentration of the protein in a cell not contacted with the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))). A compound with a lower EC₅₀ value, as determined under substantially similar conditions, is a more potent inducer of degradation relative to a compound with a higher EC₅₀ value. In some embodiments, a EC₅₀ value can be determined (e.g., using HiBiT detection) in vitro or in vivo (e.g., in tumor cells (e.g., cell lines such as A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RII, RL, SU-DHL-4, SU-DHL-5, SU-DHL-6, SU-DHL-8, SU-DHL-10, VAL, or WSU-DLCL2; see also those disclosed in, e.g., Cardenas, Mariano G., et al. Clinical Cancer Research 23.4 (2017): 885-893 and International Publication Nos. WO 2021/080950, WO 2021/077010, and WO 2022/221673) expressing a BCL6 protein)). In some embodiments, a cell line that is not dependent on BCL6 and/or that does not have significant expression of BCL6 can be used as a control (e.g., Toledo, H929, MM.1S, or OPM2).

Potency of degradation by a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, as provided herein can be determined by a Yₘᵢₙ value. As used herein, Yₘᵢₙ refers to the ratio of trough concentration of a protein (e.g., BCL6 protein) in a cell compared to the concentration of the protein before the cell is contacted with the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or compared to the concentration of the protein in a cell not contacted with the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), expressed as a percentage. As used herein, Dₘₐₓ is 1-Yₘᵢₙ. Yₘᵢₙ can be measured by a HiBiT assay as described in Example B1. A compound with a lower Yₘᵢₙ value, as determined under substantially similar conditions, is a more potent inducer of degradation relative to a compound with a higher Yₘᵢₙ value. In some embodiments, a Yₘᵢₙ value can determined (e.g., using HiBiT detection), in vitro or in vivo (e.g., in tumor cells (e.g., cell lines such as A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RII, R^{L}, SU-DHL-4, SU-DHL-5, SU-DHL-6, SU-DHL-8, SU-DHL-10, VAL, or WSU-DLCL2; see also those disclosed in, e.g., Cardenas, Mariano G., et al. Clinical Cancer Research 23.4 (2017): 885-893 and International Publication Nos. WO 2021/080950, WO 2021/077010, and WO 2022/221673) expressing a BCL6 protein)). In some embodiments, a cell line that is not dependent on BCL6 and/or that does not have significant expression of BCL6 can be used as a control (e.g., Toledo, H929, MM.1S, or OPM2).

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, exhibits a Yₘᵢₙ of less than 70% (e.g., less than 50% or less than 30%) in a HiBiT based degradation assay (e.g., an assay as described in Example B1). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, exhibits a Yₘᵢₙ of less than 50% (e.g., less than 30%) in a HiBiT based degradation assay. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, exhibits a Yₘᵢₙ of less than 30% in a HiBiT based degradation assay.

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, exhibits a Yₘᵢₙ of less than 70% (e.g., less than 50% or less than 30%) in the assay described in Example B1. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, exhibits a Yₘᵢₙ of less than 50% (e.g., less than 30%) in the assay described in Example B1. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, exhibits a Yₘᵢₙ of less than 30% in the assay described in Example B1.

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, exhibits a Yₘᵢₙ of about 0% to about 70% (e.g., about 0% to about 50%, about 30% to about 50%, or about 0% to about 30%) in a HiBiT based degradation assay (e.g., an assay as described in Example B1). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, exhibits a Yₘᵢₙ of about 0% to about 50% (e.g., about 30% to about 50% or about 0% to about 30%) in a HiBiT based degradation assay. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, exhibits a Yₘᵢₙ of about 0% to about 30% in a HiBiT based degradation assay.

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, exhibits a Yₘᵢₙ of about 0% to about 70% (e.g., about 0% to about 50%, about 30% to about 50%, or about 0% to about 30%) in the assay described in Example B1. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, exhibits a Yₘᵢₙ of about 0% to about 50% (e.g., about 30% to about 50% or about 0% to about 30%) in the assay described in Example B1. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, exhibits a Yₘᵢₙ of about 0% to about 30% in the assay described in Example B1.

It will be understood that the effect of protein degradation typically increases over time, though the appearance of degradation (e.g., as expressed by the percentage degradation compared to a control, or the parameters Yₘᵢₙ, DC₅₀, and/or Dₘₐₓ) is affected by the resynthesis rate of the protein. It is common in the art to examine degradation after a specified period of time, such as 6 hours, 12 hours, 18 hours, 1 day, 2 days, 3 days, 5 days, 10 days, or more. For example, degradation can be expressed as the percent degradation after 24 hours.

Exemplary assays for validating the degradation-inducing mechanism of a compound as provided herein are known in the art and are described, for example, in Wu, et al. Nature Structural & Molecular Biology 27.7 (2020): 605-614.

Degradation assays can be used to quantify both on- and off-target degradation-inducing effects of compounds, such as those provided herein. Exemplary assays include, quantitative immunoblotting, other immunoassays (e.g., MesoScale Discovery (MSD) immunoassays), homogenous time resolved florescence (HTRF), and HiBiT. In some embodiments, cells can be contacted with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, incubated, and then the lysate can be prepared for gel electrophoresis (e.g., SDS-PAGE), followed by immunoblotting and quantification compared to a control (e.g., a DMSO-treated control). As another example, a cell line can be engineered to express a HiBiT-tagged BCL6 protein, and the amount of fluorescence observed when the complementary LgBiT peptide is added can be compared between cells treated with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, and a control (e.g., a DMSO-treated control). See, for instance, Example B1. In some embodiments, off-target degradation inducing effects can be assessed for the proteins Eukaryotic peptide chain release factor GTP-binding subunit ERF3A (GSPT1), Ikaros (IKZF1), Helios (IKZF2), Aiolos (IKZF3), and/or casein kinase I isoform alpha (CKla). See also, e.g., International Publication Nos. WO 2018/215798; WO 2018/215801; WO 2020/104820; McCoull, William, et al., ACS Chemical Biology 13.11 (2018): 3131-3141.Bellenie, Benjamin R., et al., Journal of Medicinal Chemistry 63.8 (2020): 4047-4068; Lloyd, Matthew G., et al., Journal of Medicinal Chemistry 64.23 (2021): 17079-17097.

Binding affinity of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, as provided herein to BCL6 can be determined by, for example, a binding IC₅₀ or Kᵢ value (e.g., using a competition assay), or by a K_{D} value (e.g., using a biophysical assay). A compound with a lower binding IC₅₀ value, as determined under substantially similar conditions, is a more potent binder relative to a compound with a higher binding IC₅₀ value. A compound with a lower binding Kᵢ value, as determined under substantially similar conditions, is a more potent binder relative to a compound with a higher binding Kᵢ value. Similarly, a compound with a lower K_{D} value, as determined under substantially similar conditions, is a more potent binder relative to a compound with a higher K_{D} value. For example, a K_{D} value can be determined by surface plasmon resonance (SPR) or biolayer interferometry; see, e.g., Guo, Weikai, et al., Journal of Medicinal Chemistry 63.2 (2020): 676-695; Lloyd, Matthew G., et al., Journal of Medicinal Chemistry 64.23 (2021): 17079-17097 and International Publication Nos. WO 2019/153080; WO 2019/119144; and WO 2019/119145.

The ability of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, as provided herein to inhibit BCL6 can be determined using an IC₅₀ value. A compound with a lower IC₅₀ value, as determined under substantially similar conditions, is a more potent inhibitor relative to a compound with a higher IC₅₀ value. For example, an IC₅₀ value can be calculated using a FRET (e.g., Homogeneous Time Resolved Fluorescence (HTRF)) assay, where a tagged (e.g., His-tagged) BCL6 protein and tagged (e.g., fluorophore-tagged (e.g., Alexa-Fluor633)) corepressor peptide (e.g., BCOR) are incubated in the presence of compounds of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), and subsequently, the FRET ratio (relative to appropriate controls) can be measured using an appropriate FRET pair (e.g., with an antibody that recognizes the tagged BCL6 protein (e.g., anti-His-Terbium cryptate)). See, e.g., International Publication Nos. WO 2018/108704; WO 2018/215798; WO 2019/197842; WO 2020/104820; WO 2021/074620. As another example, an IC₅₀ value can be calculated using an enzyme-linked immunosorbent assay (ELISA) using a tagged (e.g., biotinylated) corepressor peptide (e.g., BCOR) immobilized on a substrate and a tagged (e.g., FLAG-tagged) BCL6 (e.g., a domain, such as the BTB domain, thereof), where compounds of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))) can be used to prevent the interaction between the corepressor peptide and BCL6, and the interaction between the corepressor peptide and BCL6 can be measured using an antibody to the BCL6 construct (e.g., anti-FLAG antibody). See, e.g., Kamada, Yusuke, et al., Journal of Medicinal Chemistry 60.10 (2017): 4358-4368. As yet another example, an IC₅₀ value can be calculated using a florescence polarization assay with a fluorescently-tagged corepressor peptide (e.g., SMRT) where compounds of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))) can be used to prevent the interaction between the corepressor peptide and BCL6. See, e.g., International Publication No. WO 2019/119144. As another example, a cellular IC₅₀ value can be calculated using a BRET (Bioluminescence Resonance Energy Transfer) assay, where vectors encoding BCL6 and a corepressor peptide (e.g., SMRT), complementarily fused with NanoLuc or HaloTag, can be inserted into cells. The cells can be treated with compounds of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or pharmaceutically acceptable salts thereof, to determine the effect of the compounds on inhibiting the BCL6-corepressor interaction. See, e.g., International Publication Nos. WO 2018/215798 and WO 2019/197842. In another example, an IC₅₀ value for the inhibition of BCL6 repressor function can be calculated using a luciferase assay, where cells are engineered to express luciferase under the control of one or more BCL6 repressor sites, and the cells can be incubated with compounds of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or pharmaceutically acceptable salts thereof, to determine the effect of the compounds on the function of the BCL6 repressor. See, e.g., International Publication Nos. WO 2019/119144; WO 2019/119145; WO 2019/153080.

Another exemplary way of evaluating the effect of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is to measure the induction (e.g., fold induction) of genes that are typically repressed by BCL6 (e.g., p53, ATR, CXCR3, CD69, and CDKN1A) using a method such as RT-PCR. See, e.g., Guo, Weikai, et al., Journal of Medicinal Chemistry 63.2 (2020): 676-695.

An exemplary assay for determining the potency of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, includes measuring the effect of the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, on cell proliferation and/or viability. Cell proliferation assays can be performed in a number of formats, including 2D and 3D. Similarly, a cell proliferation assay can be performed with any appropriate cell line, including, for example, A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RII, RL, SU-DHL-4, SU-DHL-5, SU-DHL-6, SU-DHL-8, SU-DHL-10, VAL, or WSU-DLCL2. In some embodiments, a cell line that is not dependent on BCL6 and/or that does not have significant expression of BCL6 can be used as a control (e.g., Toledo, H929, MM.1S, or OPM2). As an illustrative example, a 3D cell proliferation assay can include growing cells in a 3D medium, contacting the cells with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, measuring the cellular proliferation using an appropriate reagent (e.g., CELLTITERGLO® 3D), and then comparing the signal from an experiment with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, to the signal from a control experiment (e.g., lacking the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof). As another illustrative example, a 2D cell proliferation assay can include plating cells onto a growth surface, optionally letting the cells grow for a period of time, contacting the cells with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, measuring the cellular proliferation using an appropriate reagent (e.g., CELLTITERGLO®), and then comparing the signal from an experiment with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, to the signal from a control experiment (e.g., lacking a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof). Additional cell viability assays include MTT assays, which are colorimetric assays based on the reduction of the tetrazolium dye MTT (3-(4,5-dimethylthiazol-2-yl)-2,5-diphenyltetrazolium bromide) to the insoluble purple formazan, and other similar assays based on related tetrazolium salts, ATPlite assays, and other methods are known in the art. See, for instance, Example B2. See also, e.g., Guo, Weikai, et al., Journal of Medicinal Chemistry 63.2 (2020): 676-695; McCoull, William, et al. ACS Chemical Biology 13.11 (2018): 3131-3141; Lloyd, Matthew G., et al. Journal of Medicinal Chemistry 64.23 (2021): 17079-17097; Bellenie, Benjamin R., et al. Journal of Medicinal Chemistry 63.8 (2020): 4047-4068; and International Publication Nos. WO 2018/215798; WO 2018/215801; WO 2018/219281; WO 2019/119145; WO 2019/153080; and WO 2020/104820.

A cell viability assay can be used to measure the effect of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, on cell death. For example, cells expressing BCL6 protein (e.g., A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RII, RL, SU-DHL-4, SU-DHL-5, SU-DHL-6, SU-DHL-8, SU-DHL-10, VAL, or WSU-DLCL2 cells) can be incubated with various concentrations of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, then exposed to a detection reagent (e.g., using a CELLTITER-GLO® Cell Viability Assay kit) to determine cell viability. In some embodiments, the effect on cell viability can be compared to a cell line that is not dependent on BCL6 and/or that does not have significant expression of BCL6 can be used as a control (e.g., Toledo, H929, MM.1S, or OPM2).

A cell viability assay can be used to measure the effect of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, on cell death in combination with an additional therapeutic agent. For example, cells expressing BCL6 protein (e.g., A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RII, RL, SU-DHL-4, SU-DHL-5, SU-DHL-6, SU-DHL-8, SU-DHL-10, VAL, or WSU-DLCL2 cells) can be incubated (e.g., for 72 hours or for 120 hours) in a 7×7 dose matrix at various concentrations of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, and an additional therapeutic agent (e.g., any of the additional therapeutic agents described herein) (e.g., half-log diluted from 316 to 1 nM), then exposed to a detection reagent (e.g., using a CELLTITER-GLOR Cell Viability Assay kit) to determine cell viability. The combination activity can be assessed by the Bliss independence model: negative values as indication of antagonism, positive as synergy, and a value of zero as additive activity. Bliss scores in the dose matrix can be added up to give a “Bliss sum” value to reflect the overall synergy activity of the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, and the additional therapeutic agent in each cell line. In some embodiments, a cell line that is not dependent on BCL6 and/or that does not have significant expression of BCL6 can be used as a control (e.g., Toledo, H929, MM.1S, or OPM2).

As another example, the potency and/or efficacy of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can be evaluated in an animal model, for example, a cell line-derived (CDX) xenograft model (e.g., using an established cancer cell line such as DB, DoHH2, OCI-Ly1, OCI-Ly7, RL, Pfeiffer, SU-DHL-5, SU-DHL-6, WSU-DLCL2, REH, BALL-1, RS4;11, SEMK2, KOPN8, NALM-6, KASUMI-2, RCH-ACV, SUP-B15, BV-173, TOM-1, NALM-20, NALM-21, MUTZ-5, or MHH-CALL-4 (e.g., OCI-Ly1, OCI-Ly7, SU-DHL-5, SU-DHL-6, WSU-DLCL2, DB, RL, Pfeiffer, or DoHH2)), a genetically engineered mouse model (GEMM) or a patient-derived xenograft (PDX) model. For example, a PDX model can be run in immunodeficient mice (e.g., athymic nude, outbred homozygous (e.g., Crl: NU (NCr)-Foxn1nu) or Fox Chase SCID (CB17/Icr-Prkdcscid/IcrIcoCrl), mice). The mice can be female, 6-12 weeks old at tumor implantation and have access to food and water ad libitum. Approximately 70 mg of a tumor can be implanted subcutaneously in the right flank of each mouse. Following implantation, tumors can be measured weekly and once the tumor volumes reach 150-300 mm³, the mice can be randomized into treatment and control groups. In some embodiments, one or more experimental arms can be added to evaluate pharmacokinetics and/or pharmacodynamics. The mice can be treated with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, (e.g., via IP or PO administration) and optionally an additional therapy or therapeutic agent (e.g., any of the additional therapies or therapeutic agents described herein). Throughout the study, health condition, body weight and tumor volumes of the mice can be recorded on a weekly basis. The mice can be sacrificed at 28 days or when the tumor reaches 1 cm³, and the tumors can be evaluated (e.g., by tumor weight, by tumor volume). At the end of each study the Best Response can be calculated for each treatment arm. Best Response is defined as the minimum value of ΔVolumeₜ for t≥10 days. Best Responses between the control arm(s) and the treatment arm(s) can be compared to determine if the treatment(s) work better than the control(s). In some embodiments, tumor samples can also be collected at the end of each study and relevant proteins (e.g., BCL6) can be measured to determine if the treatment has a better protein modulation profile compared to a control. In some embodiments, tumor samples and/or blood samples can also be collected at the end of each study and analyzed for altered gene expression activity (e.g., altered ARID3A, ARID3B, ATR, B2M, BANK1, BATF, BCL11A, BCL2, BCL2A1, BLIMP1, BMI1, CASP8, CCND1, CCND2, CCR6, CCR7, CD38, CD44, CD69, CDKN1A, CDKN1B, CFLAR/FLIP, CHEK1, CXCR4, CXCR5, DR5, EBI2, ETV6, FCMR, FGD4, ID2, IFITM1, IFITM2, IFNAR2, IFNGR1, IL10, IL10RB, IL7R, CXCL10, IRF1, IRF4, IRF7, IRF9, JAK3, JARID2, JUN, KLF2, LITAF, MCL1, MIP-1a, MYC, MYD88, NFKBIE, NOTCH2, PDL1, PIM1, PPP3R¹, PRDMI, PTEN, SIPRI, SHP1, STAT1, STAT3, STAT5A, TLR1, TLR4, TLR7, TLR9, TNF-R², TOX, TP53, ZEB2, and/or ZNF608 expression levels). For pharmacokinetic and pharmacodynamic studies, tumor and/or blood samples from the mice can be obtained at the same or different time points than efficacy studies. For example, for pharmacokinetic and pharmacodynamic studies, tumor and/or blood samples from the mice can be obtained at Day 1, 3 and/or 5, with collections at 6, 12 and/or 24 hours post-dosing, and relevant proteins can be measured in the tumor samples and pharmacokinetic studies can be performed on the blood samples or a portion thereof (e.g., plasma). In some embodiments, the PDX is a model of B-ALL (e.g., Philadelphia chromosome positive B-ALL, Philadelphia chromosome negative B-ALL, or B-ALL with an MLL-Af4 fusion, an MLL-Af6 fusion, an MLL-Af9 fusion, an MLL-ENL fusion, or an MLL-PTD fusion), DLBCL, FL, MCL, Burkitt lymphoma, or peripheral T-cell lymphoma (PTCL) (e.g., PTCL with a T follicular helper phenotype (PTPCL-TFH), angioimmunoblastic T-cell lymphoma (AITL), or PTCL not otherwise specified (PTCL-NOS)). See, e.g., Guo, Weikai, et al., Journal of Medicinal Chemistry 63.2 (2020): 676-695.

In some embodiments, the compounds of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or pharmaceutically acceptable salts thereof, exhibit activity in a model of an autoimmune disease. Exemplary assays can be found in, for example, International Publication Nos. WO 2020/014599 and WO 2021/074620.

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or pharmaceutically acceptable salt thereof, can be assessed for its ability to modulate (e.g., decrease) IgG antibody production and/or modulate (e.g., decrease) germinal center formation in an animal (e.g., mouse) following challenge with a T cell-dependent antigen (e.g., keyhole limpet haemocyanin (KLH)). For example, KLH can be administered to mice (e.g., C57BL/6 mice), followed by administration of the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, for a period of time (e.g., 14 days). Following sacrifice, serum samples from the mice can be analyzed for IgG specific for KLH, for example, by ELISA. Similarly, germinal centers can be detected using immunohistological staining using, e.g., peanut agglutinin (PNA). See, e.g., Example 1 of WO 2020/014599.

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can be assessed for its ability to modulate (e.g., decrease) the number of germinal center B cells in an animal (e.g., mouse) following immunization with an antigen. For example, animals (e.g., mice) can be immunized with Complete Freund's Adjuvant (CFA), and after a period of time (e.g., 8 days), the animals can be sacrificed and the spleens harvested. The spleens can be processed into suspension and cultured in the presence of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or pharmaceutically acceptable salt thereof. The cells can then be analyzed, for example, for the number of germinal center B cells (e.g., by flow cytometry using the lineage markers GL7 and CD95). See, e.g., WO 2021/074620.

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or pharmaceutically acceptable salt thereof, can be assessed for its ability to improve one or more symptoms, biomarkers, or other signs of an autoimmune disease in an animal model of the autoimmune disease.

Experimental autoimmune encephalitis (EAE) can be used as an animal model of inflammatory diseases of the CNS, including multiple sclerosis (MS) and neuromyelitis optica. In some cases, EAE can be induced in animals (e.g., mice), for example, via immunization with recombinant human myelin oligodendrocyte glycoprotein (MOG) or a fragment thereof (e.g., MOG1-125), myelin basic protein, and/or proteolipid protein. Following induction of EAE, the animals can be treated with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof. The animals can be evaluated by, for example, EAE severity, B cell depletion, or both. See, e.g., Constantinescu, Cris S., et al. British Journal of Pharmacology 164.4 (2011): 1079-1106; Monson, Nancy L., et al. PloS One 6.2 (2011): e17103.

In some embodiments, the autoimmune disease is anti-synthetase syndrome. For example, a model of anti-synthetase syndrome, can be induced in susceptible mice (e.g., C₅₇BL/6, B6.G7, and/or NOD.Idd3/5) by immunization with histidyl-tRNA synthetase (e.g., murine histidyl-tRNA synthetase or human histidyl-tRNA synthetase), or a fragment thereof. Treatment with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, may begin at immunization and continue for a period of time (e.g., short term (e.g., 10-14 days) or long term (e.g., 8-16 weeks)), then the mice can be sacrificed. The mice can be evaluated, for example, for generation of histidyl-tRNA synthetase-specific antibody (e.g., via immunoprecipitation, ELISA, and/or flow cytometry), tissue (e.g., lung and/or muscle) inflammation (e.g., by pathologist review), or a combination thereof. See e.g., Katsumata, Yasuhiro, et al. Journal of Autoimmunity 29.2-3 (2007): 174-186; Katsumata, Yasuhiro, et al. Journal of Autoimmunity 29.2-3 (2007): 174-186; Ascherman, Dana P. Current Rheumatology Reports 17 (2015): 1-7; and Konishi, Risa, Yuki Ichimura, and Naoko Okiyama. Immunological Medicine 46.1 (2023): 9-14.

In some embodiments, the autoimmune disease is arthritis (e.g., rheumatoid arthritis or inflammatory arthritis). For example, a mouse model of rheumatoid arthritis, collagen induced arthritis (CIA), can be induced in susceptible mice (e.g., DBA/1 or HLA-DR) by immunization with type II collagen (CII). Treatment with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, may begin at immunization and continue for a period of time (e.g., 6 weeks). The mice can be evaluated, for example, for clinical scores (e.g., inflammation of an arthritic limb, foot thickness, paw volume (e.g., using a plethysmometer), or a combination thereof), generation of CII-specific antibody, B cell depletion, or a combination thereof. See e.g., Example 2 of WO 2020/014599; Brand, David D., et al. Nature Protocols 2.5 (2007): 1269-1275. Additional animal models of arthritis (e.g., inflammatory arthritis) are known in the art, such as K/BxN mice. In some embodiments, such mice can be treated with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, and evaluated, for example, in similar ways to CII-immunized mice, or additionally, titering for autoantibodies, such as those against glucose-6-phosphate isomerase. See, e.g., Huang, Haochu, Christophe Benoist, and Diane Mathis. Proceedings of the National Academy of Sciences 107.10 (2010): 4658-4663; and Pigott, Elizabeth, and Laura Mandik-Nayak. Arthritis & Rheumatism 64.7 (2012): 2169-2178.

In some embodiments, the autoimmune disease is graft-versus-host disease (e.g., chronic graft-versus-host disease). In some cases, an animal model of graft-versus-host disease can be in animals (e.g., mice) conditioned with high-dose cyclophosphamide and lethal total-body irradiation (TBI) rescued with bone marrow optionally including allogeneic splenocytes or purified T cells. Administration of the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, for a period of time can follow. The animals can be evaluated by, for example, pulmonary function tests, by immunohistochemistry to evaluate the presence of autoantibodies, or by examining sections of the spleen for germinal centers, including for example, examining for B cell depletion (e.g., germinal center B cell depletion). See, e.g., Srinivasan, Mathangi, et al. Blood, The Journal of the American Society of Hematology 119.6 (2012): 1570-1580; and Paz, Katelyn, et al. Blood, The Journal of the American Society of Hematology 133.1 (2019): 94-99. See also, e.g., Dubovsky, Jason A., et al. The Journal of Clinical Investigation 124.11 (2014): 4867-4876.

In some embodiments, the autoimmune disease is IgG4-related disease (IgG4-RD). In some cases, an animal model of IgG4-RD can be generated by injecting mice with IgGs (e.g., IgG1 and/or IgG4) derived from human IgG4-RD patients. Such mice can be treated with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof. The animals can be evaluated by measuring, for example, pancreatic and/or salivary tissue damage, B cell depletion, or both. See, e.g., Shiokawa, Masahiro, et al. Gut 65.8 (2016): 1322-1332.

In some embodiments, the autoimmune disease is lupus (e.g., lupus erythematosus). In some cases, an animal model of lupus is MRL/Ipr mice, which can display high expression of Tfh-associated molecules such as ICOS, PD-1, BCL-6, and IL-21 and produce autoantibodies to nuclear components, develop nephritis, arthritis, and skin lesions. Such mice can be treated with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof. The animals can be evaluated by, for example, measuring B cell depletion (e.g., germinal center B cell depletion), identifying the presence and/or severity of glomerulonephritis, autoantibody titers (e.g., anti-RNA antibody titers, anti-nuclear antibody titers, and/or anti-dsDNA antibody titers), IL-21 expression, and/or amount of activated CD4+ T cells. See, e.g., Ahuja, Anupama, et al. The Journal of Immunology 179.5 (2007): 3351-3361; Shen, Chunxiu, et al. Journal of Cellular and Molecular Medicine 25.17 (2021): 8329-8337; and Marinov, Anthony D., et al. Arthritis & Rheumatology 73.5 (2021): 826-836. Additional animal models of lupus are known in the art, such as NZB/W mice, which can be treated with a compound of Formula (I) (e.g., Formula (1-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, and evaluated in similar ways to the MRL/Ipr mice. See, e.g., Wang, Wensheng, et al. The Journal of Immunology 192.7 (2014): 3011-3020; and Kansal, Rita, et al. Science Translational Medicine 11.482 (2019): eaav1648.

In some embodiments, the autoimmune disease is myasthenia gravis (e.g., muscle-specific tyrosine kinase (MuSK) positive myasthenia gravis). For example, an animal model (e.g., a rat model, a mouse model, or a rabbit model) of myasthenia gravis can be generated by immunizing the animal with acetylcholine receptor from Torpedo (e.g., Torpedo californica) or Electrophorus (e.g., Electrophorus electricus) electric organs, or recombinant acetylcholine receptor protein or fragments thereof. Treatment with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can begin, for example, approximately 4 weeks after immunization, but can sometimes begin earlier or later. The rodents can be evaluated, for example, for clinical scores (e.g., grasping and/or lifting of a weight, while looking for tremor, hunched posture, muscle strength, and signs of fatigue), body weight, compound muscle action potential (e.g., using electromyography), anti-acetylcholine receptor antibodies, B cell depletion, or a combination thereof. See, e.g., Mori, Shuuichi, et al. The American Journal of Pathology 180.2 (2012): 798-810; Xin, Ning, et al. Molecular and Cellular Neuroscience 58 (2014): 85-94; and Losen, Mario, et al. Experimental Neurology 270 (2015): 18-28.

In some embodiments, the autoimmune disease is multiple sclerosis (MS). In some embodiments, the MS is clinically isolated syndrome (CIS), relapsing-remitting MS (RRMS), primary progressive MS (PPMS), or secondary progressive MS (SPMS). In some embodiments, animal models of MS can be based on EAE, for example, relapsing-remitting EAE in SJL/J mice (e.g., via immunization with proteolipid protein (PLP)₁₃₉₋₁₅₁), chronic EAE in C57BL/6J mice (e.g., via immunization with MOG₃₅₋₅₅), or EAE in transgenic mice (e.g., via a T cell clone expressing Va and VB chains reacting specifically to MOG₃₅₋₅₅, or a B cell heavy chain knock-in mouse). In some embodiments, the animal (e.g., mouse) model is generated via infection with a picornavirus, such as Theiler's murine encephalitis virus. In some embodiments, the animal (e.g., mouse) model is generated by feeding C57BL/6 mice with cuprizone (e.g., 0.2% for 6 weeks). In some embodiments, the animal (e.g., mouse) model is generated via lysolecithin injection (e.g., in SJL/J mice). Treatment with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can be followed, for example, by analysis of the animals for levels of myelin-specific T cells, B cell depletion, reduction of inflammatory lesions, axonal degeneration, protection or reversal of cuprizone or lysolecithin-induced demyelination, or a combination thereof. See, e.g., Procaccini, Claudio, et al. European Journal of Pharmacology 759 (2015): 182-191.

In some embodiments, the autoimmune disease is neuromyelitis optica (NMO). In some cases, animal models of NMO include administration of antibodies against astrocyte water channel aquaporin-4 (AQP4), called AQP4-IgG or NMO-IgG, to various CNS tissues, such as the brain. In some embodiments, the administration of the antibodies is to an animal with EAE. The AQP4-IgG may be recombinant or derived from human NMO patients. Treatment with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or pharmaceutically acceptable salt thereof, can be followed, for example, by analysis of the sacrificed animal for slowing or reversal of astrocyte demyelination, number of macrophages, number of activated microglia, number of activated neutrophils, B cell depletion, or a combination thereof. See, e.g., Bennett, Jeffrey L., et al. Annals of Neurology: Official Journal of the American Neurological Association and the Child Neurology Society 66.5 (2009): 617-629; Saini, Harleen, et al. BMC Neurology 13.1 (2013): 1-9; Oji, Satoru, et al. PloS One 11.3 (2016): e0151244; Peschl, Patrick, et al. Journal of Neuroinflammation 14.1 (2017): 1-14; and Duan, Tianjiao, and Alan S. Verkman. Brain Pathology 30.1 (2020): 13-25.

In some embodiments, the autoimmune disease is pemphigus (e.g., pemphigus vulgaris). In some cases, an animal (e.g., mouse) model of pemphigus can be generated using adoptive transfer of peripheral lymphocytes from Dsg3 knockout animals to immune-deficient but desmoglein 3-expressing recipient mice to create an artificial immune state in the recipient animals. In some cases, an animal (e.g., mouse) model of pemphigus can be generated using an animal with a MHC class II-null background that expresses the pemphigus-associated HLA-DRB1*0402 allele, which after immunization with recombinant human DSG3, produce anti-human DSG3 antibodies. Treatment of these models with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can be followed, for example, by analysis of the B cell repertoires of the animals for anti-desmoglein B-cell clones, B cell depletion, or both. See, e.g., Kasperkiewicz, Michael, et al. Nature Reviews Disease Primers 3.1 (2017): 1-18.

In some embodiments, the autoimmune disease is Sjogren's syndrome. In some cases, an animal model of Sjogren's syndrome is a NOD mouse, which sometimes has further genetic manipulation or crosses (e.g., NOD.B10.H2^{b} or C57BL/6.NOTDc3.NODclt) to recapitulate the symptoms of the syndrome. Such mice can be treated with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof. The animals can be evaluated, for example, for salivary and lacrimal gland secretory flow rates, salivary protein content, autoantibodies to exocrine gland proteins (e.g., anti-Sjogren's syndrome A (SSA) antibodies, anti-Sjogren's syndrome B (SSB) antibodies, and/or anti-muscarinic acetylcholine 3 receptor (M3R) antibodies), B cell depletion, or a combination thereof. Robinson, Christopher P., et al. Arthritis & Rheumatism 41.1 (1998): 150-156; Cha, Seunghee, et al. “Arthritis & Rheumatism 46.5 (2002): 1390-1398; and Ohno, Seiji, et al. Autoimmunity 45.7 (2012): 540-546. Additional animal models of Sjogren's syndrome are known in the art and include, for example, NFS/sld mice, IQI/Jic mice, Aly/aly mice, and mice immunized with M3R peptides, which can be evaluated, in some cases, for the same parameters as NOD mice. See, e.g., Iizuka, Mana, et al. Journal of Autoimmunity 35.4 (2010): 383-389; and Park, Young-Seok, Adrienne E Gauna, and Seunghee Cha. Current Pharmaceutical Design 21.18 (2015): 2350-2364.

The pharmacokinetic parameters of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can be evaluated in an animal model, for instance, a mouse model, a rat model, a dog model, or a nonhuman primate (e.g., cynomolgus monkey) model. Pharmacokinetics (PK) studies can be conducted in an animal model (e.g., male CD-1 mice) by two exemplary delivery routes: intravenous (IV) injection and oral (PO), for example, oral gavage. Animals in the IV group (e.g., n=3) are allowed free access to food and water; animals in the PO group are allowed free access to food or are fasted for 6-8 hours prior to dosing. A compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can be formulated in solution for the IV route and solution or suspension for the PO route. On the day of the experiment, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can be administered via vein injection (e.g., at 1 mg/kg) for IV route or via oral gavage (e.g., at 3 to 90 mg/kg, or 3 to 10 mg/kg, such as 10 mg/kg) for PO route. In some cases, the animals can be orally pre-dosed with a cytochrome P450 inhibitor (e.g., 1-aminobenzotriazole) prior to (e.g., 16 hours prior to) dosing the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof. Blood samples can be collected via serial bleeding (e.g., at 8 timepoints from 0.83 to 24 hours post dose). At each timepoint, blood (e.g., about 30 μL to about 125 μL, or about 75 μL to about 125 μL) can be collected (e.g., via the saphenous vein) in a tube containing an anti-coagulant (e.g., K₂EDTA). Blood samples can be put on wet ice and centrifuged (e.g., at 2000×g for 4-10 minutes) to obtain plasma samples. Plasma samples can be diluted (e.g., with an equal volume of pH 3.0 phosphate buffer or with an equal volume of pH 5.0 sodium citrate) and submitted to LC-MS/MS for sample analysis. Pharmacokinetics parameters, including clearance (CL), volume of distribution (V_{d}), maximum plasma concentration (Cₘₐₓ), time of maximum plasma concentration (tₘₐₓ), half-life (t_{1/2}), area under the curve (AUC), and oral bioavailability (% F) can be calculated using a non-compartmental model.

In some embodiments, the % F for a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is at least 4%. In some embodiments, the % F for a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is at least 10%. In some embodiments, the % F for a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is at least 20%. In some embodiments, the % F for a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is at least 30%. In some embodiments, the % F for a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is at least 40%. In some embodiments, the % F for a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is at least 60%. In some embodiments, the % F for a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is at least 80%. In some embodiments, the % F for a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is about 4% to about 90% (e.g., about 4% to about 80%, about 4% to about 60%, about 4% to about 40%, about 4% to about 20%, about 4% to about 10%, about 20% to about 40%, about 40% to about 60%, about 60% to about 80%, or about 70% to about 90%). In some embodiments, the % F for a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is about 4% to about 20%. In some embodiments, the % F for a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is about 20% to about 40%. In some embodiments, the % F for a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is about 40% to about 60%. In some embodiments, the % F for a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is about 50% to about 80%.

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is not a substrate of a human cytochrome P450 enzyme. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is not a substrate of a human cytochrome P450 enzyme where ≥25% of clearance is attributed to that enzyme. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is not an inhibitor and/or an inducer of one or more human cytochrome P450 enzymes. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is not an inhibitor and/or an inducer of one or more human cytochrome P450 enzymes, where the IC₅₀ and/or EC₅₀ for the one or more human cytochrome P450 enzymes, respectively, is at a concentration to be significantly greater than the estimated free fraction concentration of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, at a clinically relevant dose.

Exemplary human cytochrome P450 enzymes include those in the CYP1, CYP2, and CYP3 families. For example, any one of CYP1A1, CYP1A2, CYP1B1, CYP2A6, CYP2B6, CYP2C₈, CYP2C9, CYP2C18, CYP2C19, CYP2D6, CYP2J2, CYP2S1, CYP2E1, CYP3A4, and CYP3A5. In some embodiments, no single cytochrome P450 enzyme is responsible for greater than or equal to 25% of the elimination of the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof. Cytochrome P450 inhibition and/or inducing activity can be determined using any appropriate assay, such as those described in the guidance document “In Vitro Drug Interaction Studies-Cytochrome P450 Enzyme- and Transporter-Mediated Drug Interactions” provided by the U.S. F.D.A. in January 2020. For example, evaluation of cytochrome P450 inhibition can be performed in in vitro studies, in both a reversible and time-dependent manner. In an in vitro inhibition study, the ratio of intrinsic clearance values of a probe substrate for an enzymatic pathway in the absence and in the presence of the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can be calculated; this ratio is referred to as R₁ for reversible inhibition, where R₁=1+(I_{max,u}/K_{i,u}), and I_{max,u} is the maximal unbound plasma concentration of the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, and K_{i,u} is the unbound inhibition constant determined in vivo. Specifically, for CYP3A, R_{1,gut} can be calculated where R_{1,gut}=1+ (I_{gut}+K_{i,u}), and where I_{gut} is the intestinal luminal concentration of the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, calculated as the dose/250 mL. The time-dependent inhibition ratio R² can similarly be calculated, where R₂=(k_{obs}+k_{deg})/k_{deg}, and k_{obs} is the observed (apparent first order) inactivation rate of the affected cytochrome P450 calculated by k_{obs}=(k_{inact}*50*I_{max,u})/(K_{I,u}+50*I_{max,u}), k_{deg} is the apparent first-order degradation rate constant of the affected cytochrome P450, K_{I,u} is the unbound concentration of the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, causing half maximal inactivation, and k_{inact} is the maximal inactivation rate constant. If R₁≥1.02, R₂≥1.25, and/or R_{1,gut}≥11, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, may be an inhibitor of a cytochrome P450, and the drug-drug interaction (DDI) potential can be further investigated using mechanistic models and/or conducting a clinical DDI study with a sensitive index substrate. For example, evaluation of cytochrome P450 induction can be performed via the fold-change method, wherein the fold-change in cytochrome P450 enzyme mRNA levels when incubated with the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, by using a cutoff determined from known positive and negative controls to calibrate the system. For example, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is interpreted as an inducer if: (1) it increased mRNA expression of a cytochrome P450 enzyme in a concentration-dependent manner; and (2) the fold change of cytochrome P450 mRNA expression relative to the vehicle control is ≥2-fold at the expected hepatic concentrations of the drug. As another example, evaluation of cytochrome P450 induction can be performed by a correlation method, wherein correlation methods are used to predict the magnitude of a clinical induction effect (e.g., AUC ratio of an index substrate in the presence and absence of inducers) of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, according to a calibration curve of relative induction scores (RIS) or I_{max,u}/EC₅₀ for a set of known inducers of the same cytochrome P450. If the predicted magnitude is more than a predefined cut-off (e.g., AUC ratio≤0.8), a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is considered to have induction potential in vivo.

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is not a hERG inhibitor. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, inhibits hERG with an IC₅₀ of greater than 60 nM (e.g., greater than 100 nM, 300 nM, 500 nM, 1 μM, 3 μM, 5 μM, 10 M, 20 μM, or 30 μM). For example, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, inhibits hERG with an IC₅₀ of greater than 500 nM (e.g., 1 μM, 3 μM, 5 μM, 10 μM, 20 μM, or 30 uM). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, inhibits hERG with an IC₅₀ of greater than 1 μM (e.g., greater than 3 μM, 5 μM, 10 μM, 20 μM, or 30 μM). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, inhibits hERG with an IC₅₀ of greater than 10 μM (e.g., greater than 20 μM or 30 μM). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, inhibits hERG with an IC₅₀ of greater than 30 μM.

Heterobifunctional degraders can, in some cases, induce the degradation of off-target proteins. For heterobifunctional degraders that utilize CRBN, common off-target proteins that can be degraded include GSPT1, IKZF1, IKZF2, IKZF3, and/or CK1α. This degradation is generally believed to be due to the E3 binding moiety of the heterobifunctional degrader facilitating ternary complex formation between the off-target protein and CRBN. GSPT1 is a translation termination factor, and CK1α is a kinase that is involved in many key cellular processes including cell cycle progression and chromosome segregation; these are both commonly essential genes, so undesired degradation of either or both may lead to nonspecific cytotoxicity. The IKZF proteins are zinc finger transcription factors that are involved with cell fate during hematopoiesis, and degradation of these proteins has been associated with hematotoxicity. See, e.g., Moreau, Kevin, et al. British Journal of Pharmacology 177.8 (2020): 1709-1718.

In some embodiments, the compounds of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or pharmaceutically acceptable salts thereof, can exhibit potent and selective induction of degradation of a BCL6 protein. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutically acceptable salt thereof, can selectively target a BCL6 protein for degradation over a second protein (e.g., GSPT1, IKZF1, IKZF2, IKZF3, CK1α, C6orf132, CAMP (cathelicidin antimicrobial peptide), CCNA2 (cyclin-A²), FSP1 (ferroptosis suppressor protein 1, also known as AIFM2), JCHAIN (immunoglobin J chain), NLRP7 (NACHT, LRR, PYD domains-containing protein 7), PTTG1 (securin), and/or TPX2 (targeting protein for Xklp2)). CAMP is an antimicrobial protein that is an integral part of the innate immune system, and it binds to bacterial lipopolysaccharides. CCNA2 controls both the G1/S and the G2/M transition phases of the cell cycle. FSP1 is an oxidoreductase that is an inhibitor of ferroptosis. JCHAIN links two monomer units of either IgM or IgA; the J chain-joined dimer is a nucleating unit of the IgM pentamer, and the J chain-joined dimer of IgA induces dimers or larger polymers. NLRP7 Inhibits CASP1/caspase-1-dependent IL1B secretion. PTTG1 is key for chromosomal stability and negatively regulates TP53. TPX2 is required for the normal assembly of mitotic spindles.

As used herein, “selective” or “selectively”, when referring to a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, in a protein degradation assay, indicates at least a 5-fold (e.g., at least a 10-fold, at least a 25-fold, at least a 50-fold, or at least a 100-fold) superior performance in the protein degradation assay for a specified protein with reference to a comparator protein in the assay.

In some embodiments, the compounds provided herein can exhibit potency (e.g., nanomolar potency) against a BCL6 protein with minimal activity (e.g., single digit micromolar potency, for example, potency greater than 1 μM (e.g., greater than 3 μM, 5 M, 10 M, 20 μM, or 30 μM)) against a second protein. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit potent degradation of a BCL6 protein and have minimal potency in degrading (e.g., as measured by Yₘᵢₙ, DC₅₀, and/or Dₘₐₓ values) a second protein (e.g., GSPT1, IKZF1, IKZF2, IKZF3, CK1α, C6orf132, CAMP, CCNA2, FSP1, JCHAIN, NLRP7, PTTG1, and/or TPX2). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit greater induction of degradation of a BCL6 protein relative to induction of degradation (e.g., as measured by Yₘᵢₙ, DC₅₀, and/or Dₘₐₓ values) of a second protein (e.g., GSPT1, IKZF1, IKZF2, IKZF3, CK1α, C6orf132, CAMP, CCNA2, FSP1, JCHAIN, NLRP7, PTTG1, and/or TPX2). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit at least 2-fold, 3-fold, 5-fold, 10-fold, 25-fold, 50-fold, or 100-fold greater induction of degradation of a BCL6 protein relative to induction of degradation of a second protein. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit up to 1000-fold greater induction of degradation of a BCL6 protein relative to induction of degradation of a second protein. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit from about 2-fold to about 10-fold greater induction of degradation of a BCL6 protein relative to induction of degradation of a second protein (e.g., GSPT1, IKZF1, IKZF2, IKZF3, CK1α, C6orf132, CAMP, CCNA2, FSP1, JCHAIN, NLRP7, PTTG1, and/or TPX2) (e.g., as measured by Yₘᵢₙ, DC₅₀, and/or Dₘₐₓ values). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit from about 10-fold to about 100-fold greater induction of degradation of a BCL6 protein relative to induction of degradation of a second protein. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit from about 100-fold to about 1000-fold greater induction of degradation of a BCL6 protein relative to induction of degradation of a second protein. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit from about 1000-fold to about 10000-fold greater induction of degradation of a BCL6 protein relative to induction of degradation of a second protein. In some embodiments, the second protein is selected from the group consisting of GSPT1, IKZF1, IKZF2, IKZF3, CKla, C6orf132, CAMP, CCNA2, FSP1, JCHAIN, NLRP7, PTTG1, and TPX2. In some embodiments, the second protein is selected from the group consisting of GSPT1, IKZF1, IKZF2, IKZF3, and CKla. In some embodiments, the second protein is selected from the group consisting of C6orf132, CAMP, CCNA2, FSP1, JCHAIN, NLRP7, PTTG1, and TPX2. In some embodiments, the second protein is C6orf132. In some embodiments, the second protein is CAMP. In some embodiments, the second protein is CCNA2. In some embodiments, the second protein is FSP1. In some embodiments, the second protein is JCHAIN. In some embodiments, the second protein is NLRP7. In some embodiments, the second protein is PTTG1. In some embodiments, the second protein is TPX2.

In some embodiments, the compounds provided herein can exhibit potency against a BCL6 protein with similar activity against a second protein (i.e., less than 2-fold greater activity against a BCL6 protein than against a second protein and no more than 2-fold greater activity against the second protein than against the BCL6 protein). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit similar induction of degradation of a BCL6 protein relative to induction of degradation (e.g., as measured by Yₘᵢₙ, DC₅₀, and/or Dₘₐₓ values) of a second protein (i.e., less than 2-fold difference greater induction of degradation of a BCL6 protein than induction of degradation of a second protein and no more than 2-fold greater activity against the second protein than against a BCL6 protein) (e.g., GSPT1, IKZF1, IKZF2, IKZF3, CK1a, C6orf132, CAMP, CCNA2, FSP1, JCHAIN, NLRP7, PTTG1, and/or TPX2). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit less than 2-fold greater induction of degradation of a BCL6 protein relative to induction of degradation of a second protein. In some embodiments, the second protein is selected from the group consisting of GSPT1, IKZF1, IKZF2, IKZF3, CKla, C6orf132, CAMP, CCNA2, FSP1, JCHAIN, NLRP7, PTTG1, and TPX2. In some embodiments, the second protein is selected from the group consisting of GSPT1, IKZF1, IKZF2, IKZF3, and CK1α. In some embodiments, the second protein is selected from the group consisting of C6orf132, CAMP, CCNA2, FSP1, JCHAIN, NLRP7, PTTG1, and TPX2. In some embodiments, the second protein is C6orf132. In some embodiments, the second protein is CAMP. In some embodiments, the second protein is CCNA2. In some embodiments, the second protein is FSP1.

In some embodiments, the second protein is JCHAIN. In some embodiments, the second protein is NLRP7. In some embodiments, the second protein is PTTG1. In some embodiments, the second protein is TPX2.

In some embodiments, the compounds provided herein can exhibit potency against a BCL6 protein with minimal activity against a second protein (e.g., as measured by a proteomics assay, for example, a <20% reduction in protein abundance as measured in the proteomics assay described herein). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit potent degradation of a BCL6 protein and have minimal potency in degrading (e.g., as measured by abundance in a proteomic assay) a second protein (e.g., C6orf132, CAMP, CCNA2, FSP1, JCHAIN, NLRP7, PTTG, and/or TPX2) (e.g., as measured by abundance in a proteomic assay, for example, the proteomics assay as described herein). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit greater induction of degradation of a BCL6 protein relative to induction of degradation (e.g., as measured by abundance in a proteomic assay, for example, the proteomics assay as described herein) of a second protein (e.g., C6orf132, CAMP, CCNA2, FSP1, JCHAIN, NLRP7, PTTG1, and/or TPX2). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit at least 2-fold, 3-fold, 5-fold, 10-fold, 25-fold, 50-fold, or 100-fold greater induction of degradation of a BCL6 protein relative to induction of degradation of a second protein (e.g., C6orf132, CAMP, CCNA2, FSP1, JCHAIN, NLRP7, PTTG1, and/or TPX2) (e.g., as measured by abundance in a proteomic assay, for example, the proteomics assay as described herein). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit up to 1000-fold greater induction of degradation of a BCL6 protein relative to induction of degradation of a second protein (e.g., C6orf132, CAMP, CCNA2, FSP1, JCHAIN, NLRP7, PTTG1, and/or TPX2) (e.g., as measured by abundance in a proteomic assay, for example, the proteomics assay as described herein). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit from about 2-fold to about 10-fold greater induction of degradation of a BCL6 protein relative to induction of degradation of a second protein (e.g., C6orf132, CAMP, CCNA2, FSP1, JCHAIN, NLRP7, PTTG1, and/or TPX2) (e.g., as measured by abundance in a proteomic assay, for example, the proteomics assay as described herein). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit from about 10-fold to about 100-fold greater induction of degradation of a BCL6 protein relative to induction of degradation of a second protein (e.g., C6orf132, CAMP, CCNA2, FSP1, JCHAIN, NLRP7, PTTG1, and/or TPX2) (e.g., as measured by abundance in a proteomic assay, for example, the proteomics assay as described herein). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit from about 100-fold to about 1000-fold greater induction of degradation of a BCL6 protein relative to induction of degradation of a second protein (e.g., C6orf132, CAMP, CCNA2, FSP1, JCHAIN, NLRP7, PTTG1, and/or TPX2) (e.g., as measured by abundance in a proteomic assay, for example, the proteomics assay as described herein). In some embodiments, the second protein is selected from the group consisting of GSPT1, IKZF1, IKZF2, IKZF3, CKla, C6orf132, CAMP, CCNA2, FSP1, JCHAIN, NLRP7, PTTG1, and TPX2. In some embodiments, the second protein is selected from the group consisting of GSPT1, IKZF1, IKZF2, IKZF3, and CKla. In some embodiments, the second protein is selected from the group consisting of C6orf132, CAMP, CCNA2, FSP1, JCHAIN, NLRP7, PTTG1, and TPX2. In some embodiments, the second protein is C6orf132. In some embodiments, the second protein is CAMP. In some embodiments, the second protein is CCNA2. In some embodiments, the second protein is FSP1. In some embodiments, the second protein is JCHAIN. In some embodiments, the second protein is NLRP7. In some embodiments, the second protein is PTTG1. In some embodiments, the second protein is TPX2.

In some embodiments, the compounds provided herein can exhibit potency against a BCL6 protein with minimal activity against any other detectable protein (e.g., as measured by a proteomics assay, for example, a <20% reduction in protein abundance as measured in the proteomics assay described herein). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit potent degradation of a BCL6 protein and have minimal potency in degrading (e.g., as measured by abundance in a proteomic assay, for example, the proteomics assay as described herein) any other detectable protein. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit greater induction of degradation of a BCL6 protein relative to induction of degradation (e.g., as measured by abundance in a proteomic assay, for example, the proteomics assay as described herein) of any other detectable protein. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit at least 2-fold, 3-fold, 5-fold, 10-fold, 25-fold, 50-fold, or 100-fold greater induction of degradation of a BCL6 protein relative to induction of degradation of any other detectable protein (e.g., as measured by abundance in a proteomic assay, for example, the proteomics assay as described herein). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit up to 1000-fold greater induction of degradation of a BCL6 protein relative to induction of degradation of any other detectable protein (e.g., as measured by abundance in a proteomic assay, for example, the proteomics assay as described herein). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit from about 2-fold to about 10-fold greater induction of degradation of a BCL6 protein relative to induction of degradation of any other detectable protein (e.g., as measured by abundance in a proteomic assay, for example, the proteomics assay as described herein). In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit from about 10-fold to about 100-fold greater induction of degradation of a BCL6 protein relative to induction of degradation of any other detectable protein (e.g., as measured by abundance in a proteomic assay, for example, the proteomics assay as described herein).

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can exhibit potent degradation of a BCL6 protein and have minimal potency in degrading one or more additional proteins as measured by abundance in a proteomic assay. An exemplary proteomic experiment follows. OCI-Ly1 (DSMZ: ACC 722) cells are incubated with 100 nM of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or dimethyl sulfoxide (DMSO), for six hours. The cells are then washed two times with phosphate buffered saline and collected. Cells are lysed to extract total proteins, and total proteins are prepared for mass spectrometry analysis according to the protocol for the EASYPEPIM MS Sample Prep Kit (Fisher Scientific). In brief, proteins are reduced with dithiothreitol, alkylated with iodoacetamide, and digested with Trypsin and LysC enzyme. The resulting peptides are labeled with TMTPRO™ 18plex reagents (Fisher Scientific) according to the manufacturer protocol. Labeled peptides from each sample are mixed together in equal volumes, and the peptide mixture is separated by basic reverse-phase chromatography. A total of 85 fractions are combined into 18 pooled fractions. The pooled fractions are dried with a centrivap and resuspended in 5% acetonitrile, 0.1% formic acid for mass spectrometry analysis. Peptide abundance is quantified by tandem mass spectrometry using a Vanquish Neo chromatography system (Fisher Scientific) and Orbitrap FUSION™ LUMOS™ mass spectrometer (Fisher Scientific). Briefly, two micrograms of total peptides are loaded on a two-centimeter C8 trap column followed by a 50-centimeter C18 column. Data-dependent acquisition is performed to obtain peptide sequence and abundance information. Peptide and protein abundances are determined using the PROTEOME DISCOVERER™ software and the Homo sapiens proteome database (TaxID 9606), and the results are filtered to FDR<0.01. Significance thresholds are set to p-value <0.001 and abundance fold-change <50%.

Provided herein is a method of treating a cancer in a subject in need thereof, the method comprising administering to the subject a therapeutically effective amount of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition comprising a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof. In some embodiments, the subject is treatment naïve with respect to the cancer. In some embodiments, the subject has received one or more lines of previous therapy for the cancer.

Also provided herein is a method of treating a cancer in a subject in need thereof, the method comprising administering to the subject a therapeutically effective amount of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition comprising a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof as a monotherapy. In some embodiments, the subject is treatment naïve with respect to the cancer. In some embodiments, the subject has received one or more lines of previous therapy for the cancer.

Also provided herein is a method of treating a cancer in a subject in need thereof, the method comprising administering to the subject a therapeutically effective amount of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition thereof, as a monotherapy. In some embodiments, the subject is treatment naïve with respect to the cancer. In some embodiments, the subject has received one or more lines of previous therapy for the cancer.

Provided herein is use of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition thereof, for the treatment of cancer, for example, any of the cancers provided herein.

Provided herein is use of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition thereof, as a medicament for the treatment of cancer, for example, any of the cancers provided herein.

Provided herein is use of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, in the manufacture of a medicament for the treatment of cancer, for example, any of the cancers provided herein.

Provided herein is a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition thereof, for use as a medicament. Also provided herein is a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition thereof, for use as a medicament for the treatment of cancer, for example, any of the cancers provided herein.

Provided herein is a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition thereof, for use in treating a cancer, for example, any of the cancers provided herein.

As used herein, treatment of a cancer can include treatment of a primary tumor (i.e., non-metastatic cancer) (e.g., as first, second, third, or later line of therapy, including, but not limited to, the relapsed/refractory setting), treatment of a metastatic (or secondary) tumor, neoadjuvant therapy (e.g., before treatment with an additional therapy or therapeutic agent, such as surgery, radiation, chemotherapy, or a line of therapy), adjuvant therapy (e.g., following treatment with an additional therapy or therapeutic agent, such as surgery, radiation, chemotherapy, or a line of therapy), or maintenance therapy (e.g., treatment following response to an additional therapy or therapeutic agent, such as surgery, radiation, chemotherapy, or a line of therapy).

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is used in the treatment of a primary tumor. In some embodiments, the subject is treatment naïve with respect to the cancer. In some embodiments, the subject has received one or more lines of therapy for the cancer. In some embodiments, the patient has received chemotherapy, cell-based therapy (e.g., adoptive cell therapy (e.g., CAR T therapy, cytokine-induced killer cells (CIKs), natural killer cells (e.g., CAR-modified NK cells)) or antibody-armed cell therapy), or both. In some embodiments, the patient has received R-CHOP, G-CHOP, R-EPOCH, CVP, CVAD, R², R-CODOX-M, R-IVAC, DA-EPOCH-R, cell-based therapy, or two or more thereof. In some embodiments, the patient has received a rituximab-containing regimen. In some embodiments, the patient has received an obinutuzumab-containing regimen. In some embodiments, the patient has received a mosunetuzumab-containing regimen. In some embodiments, the patient has received an epcoritamab-containing regimen. In some embodiments, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is used in the treatment of a patient who has received one or more lines of systemic therapy for the cancer. In some embodiments, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is used in the treatment of a patient who has received two or more lines of systemic therapy for the cancer.

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is used in the treatment of a metastatic tumor. In some embodiments, the subject is treatment naïve with respect to the metastatic tumor. In some embodiments, the subject has received one or more lines of therapy for the secondary tumor. In some embodiments, the patient has received chemotherapy, cell-based therapy (e.g., adoptive cell therapy (e.g., CAR T therapy, cytokine-induced killer cells (CIKs), natural killer cells (e.g., CAR-modified NK cells)) or antibody-armed cell therapy), or both. In some embodiments, the patient has received R-CHOP, G-CHOP, R-EPOCH, CVP, CVAD, R², R-CODOX-M, R-IVAC, DA-EPOCH-R, cell-based therapy, or two or more thereof. In some embodiments, the patient has received a rituximab-containing regimen. In some embodiments, the patient has received an obinutuzumab-containing regimen. In some embodiments, the patient has received a mosunetuzumab-containing regimen. In some embodiments, the patient has received an epcoritamab-containing regimen. In some embodiments, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is used in the treatment of a patient who has received one or more lines of systemic therapy for the cancer. In some embodiments, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is used in the treatment of a patient who has received two or more lines of systemic therapy for the cancer.

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is used as neoadjuvant therapy. In some embodiments, the neoadjuvant therapy precedes surgery (e.g., surgical resection, such as partial surgical resection or complete, total, or full surgical resection). In some embodiments, the neoadjuvant therapy precedes radiation therapy. In some embodiments, the neoadjuvant therapy precedes chemotherapy.

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is an adjuvant therapy. In some embodiments, the patient has received chemotherapy, cell-based therapy (e.g., adoptive cell therapy (e.g., CAR T therapy, cytokine-induced killer cells (CIKs), natural killer cells (e.g., CAR-modified NK cells)) or antibody-armed cell therapy), or both. In some embodiments, the patient has received R-CHOP, G-CHOP, R-EPOCH, CVP, CVAD, R², R-CODOX-M, R-IVAC, DA-EPOCH-R, cell-based therapy, or two or more thereof. In some embodiments, the patient has received a rituximab-containing regimen. In some embodiments, the patient has received an obinutuzumab-containing regimen. In some embodiments, the patient has received a mosunetuzumab-containing regimen. In some embodiments, the patient has received an epcoritamab-containing regimen. In some embodiments, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is used in the treatment of a patient who has received one or more lines of systemic therapy for the cancer. In some embodiments, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is used in the treatment of a patient who has received two or more lines of systemic therapy for the cancer. In some embodiments, the adjuvant therapy follows surgery (e.g., surgical resection, such as partial surgical resection or complete, total, or full surgical resection). In some embodiments, the adjuvant therapy follows radiation therapy. In some embodiments, the adjuvant therapy follows chemotherapy.

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is a maintenance therapy. In some embodiments, the patient has received chemotherapy, cell-based therapy (e.g., adoptive cell therapy (e.g., CAR T therapy, cytokine-induced killer cells (CIKs), natural killer cells (e.g., CAR-modified NK cells)) or antibody-armed cell therapy), a stem cell transplant, or a combination thereof. In some embodiments, the patient has received R-CHOP, G-CHOP, R-EPOCH, CVP, CVAD, R², R-CODOX-M, R-IVAC, DA-EPOCH-R, cell-based therapy, or two or more thereof. In some embodiments, the patient has received a rituximab-containing regimen. In some embodiments, the patient has received an obinutuzumab-containing regimen. In some embodiments, the patient has received a mosunetuzumab-containing regimen. In some embodiments, the patient has received an epcoritamab-containing regimen. In some embodiments, the patient has received a stem cell transplant. In some embodiments, the patient has received a cell-based therapy (e.g., CAR T therapy). In some embodiments, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is used in the treatment of a patient who has received one or more lines of systemic therapy for the cancer. In some embodiments, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is used in the treatment of a patient who has received two or more lines of systemic therapy for the cancer.

As used herein, “monotherapy”, when referring to a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, means that the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is the only therapeutic agent or therapy (e.g., anticancer agent or therapy) administered to the subject during the treatment cycle (e.g., no additional targeted therapeutics, anticancer agents, chemotherapeutics, or checkpoint inhibitors are administered to the subject during the treatment cycle). As a person of ordinary skill in the art would understand, monotherapy does not exclude the co-administration of medicaments for the treatment of side effects or general symptoms associated with the cancer or treatment, such as pain, rash, edema, photosensitivity, pruritis, skin discoloration, hair brittleness, hair loss, brittle nails, cracked nails, discolored nails, swollen cuticles, fatigue, weight loss, general malaise, shortness of breath, infection, anemia, or gastrointestinal symptoms, including nausea, diarrhea, and lack of appetite.

As used herein, “the subject has previously received one or more therapeutic agents or therapies for the cancer” means that the subject has been previously administered one or more therapeutic agents or therapies (e.g., anticancer agent or therapy) for the cancer other than a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, during a prior treatment cycle. In some embodiments, the subject cannot tolerate the one or more therapeutic agents or therapies previously administered for the cancer. In some embodiments, the subject did not respond to the one or more therapeutic agents or therapies previously administered for the cancer. In some embodiments, the subject did not adequately respond to one or more therapeutic agents or therapies previously administered for the cancer. In some embodiments, the subject has stopped responding to the one or more therapeutic agents or therapies previously administered for the cancer. In some embodiments, a lack of response, an inadequate response, or a discontinued response can be determined by objective criteria (e.g., tumor volume, or by criteria such as RECIST 1.1). In some embodiments, a lack of response, an inadequate response, or a discontinued response can be determined by the subject's physician.

As used herein, “the subject is treatment naïve with respect to the cancer” means that the subject has not been previously administered one or more therapeutic agents or therapies for the cancer.

For any of the solid tumors described herein, the solid tumor can be primary tumors or metastatic (or secondary) tumors. As used herein, “primary” tumors are those located at the site where the tumor began to grow (i.e., where it originated). As used herein, “metastatic” (or “secondary”) tumors are those that have spread to other parts of body from the original tumor site. In some embodiments, the metastatic or secondary tumors are the same type of cancer as the primary tumor. In some embodiments, the metastatic or secondary tumors are not genetically identical to the primary tumor.

In some embodiments of any of the methods or uses described herein, the cancer is breast cancer (e.g., breast invasive carcinoma, breast invasive ductal carcinoma), central or peripheral nervous system tissue cancer (e.g., brain cancer (e.g., astrocytoma, glioblastoma, glioma, oligoastrocytoma)), endocrine or neuroendocrine cancer (e.g., adrenal cancer (e.g., adrenocortical carcinoma, pheochromocytoma, paraganglioma), multiple neuroendocrine type I and type II tumors, parathyroid cancer, pituitary tumors, thyroid cancer (e.g., papillary thyroid cancer)), eye cancer (e.g., uveal cancer (e.g., uveal melanoma)), gastrointestinal cancer (e.g., anal cancer, bile duct cancer (e.g., cholangiocarcinoma), colorectal cancer (e.g., colon adenocarcinoma, rectal adenocarcinoma, mucinous adenocarcinoma, mucinous carcinoma), esophageal cancer (e.g., esophageal adenocarcinoma), gallbladder cancer, gastrointestinal carcinoid tumor, gastrointestinal stromal tumor, liver cancer (e.g., hepatocellular carcinoma, intrahepatic bile duct cancer), pancreatic cancer (e.g., pancreatic adenocarcinoma, pancreatic islet cell cancer), small intestine cancer, or stomach cancer (e.g., stomach adenocarcinoma, signet ring cell carcinoma of the stomach)), genitourinary cancer (e.g., bladder cancer (e.g., bladder urothelial carcinoma), kidney cancer (e.g., renal clear cell carcinoma, renal papillary cell carcinoma, kidney chromophobe), prostate cancer (e.g., prostate adenocarcinoma), testicular cancer (e.g., testicular germ cell tumors), or ureter cancer), gynecologic cancer (e.g., cervical cancer (e.g., cervical squamous cell carcinoma, endocervical adenocarcinoma, mucinous carcinoma), ovarian cancer (e.g., serous ovarian cancer, ovarian serous cystadenocarcinoma), uterine cancer (e.g., uterine carcinosarcoma, uterine endometrioid carcinoma, uterine serous carcinoma, uterine papillary serous carcinoma, uterine corpus endometrial carcinoma), or vulvar cancer), head and neck cancer (e.g., ear cancer (e.g., middle ear cancer), head and neck squamous cell carcinoma, nasal cavity cancer, oral cancer, pharynx cancer (e.g., hypopharynx cancer, nasopharynx cancer, oropharyngeal cancer), hematological cancer (e.g., leukemia (e.g., chronic lymphocytic leukemia (CLL), acute lymphocytic leukemia (ALL) (e.g., Philadelphia chromosome positive ALL, Philadelphia chromosome negative ALL), acute myeloid leukemia (AML) (e.g., acute promyelocytic leukemia (APL)), chronic myeloid leukemia (CML)), lymphoma (e.g., Hodgkin lymphoma (e.g., nodular lymphocyte predominant Hodgkin lymphoma (NLPHL)), non-Hodgkin lymphoma (e.g., Burkitt lymphoma (BL), diffuse large B-cell lymphoma (DLBCL), diffuse histiocytic lymphoma (DHL), follicular lymphoma (FL), intravascular large B-cell lymphoma (IVLBCL), mantle cell lymphoma (MCL), peripheral T-cell lymphoma (PTCL) (e.g., PTCL with a T follicular helper phenotype (PTPCL-TFH), angioimmunoblastic T-cell lymphoma (AITL), or PTCL not otherwise specified (PTCL-NOS)), small lymphocytic lymphoma (SLL))), or myeloma (e.g., multiple myeloma)), Li-Fraumeni tumors, mesentery cancer (e.g., omentum cancer, peritoneal cancer), pleural cancer, respiratory cancer (e.g., larynx cancer, lung cancer (e.g., lung squamous cell carcinoma, lung adenocarcinoma, mesothelioma, non-small cell lung cancer (NSCLC)), tracheal cancer), sarcoma (e.g., bone cancer (e.g., osteosarcoma, chondrosarcoma) or soft tissue sarcoma (Ewing sarcoma, leiomyosarcoma, myxofibrosarcoma, rhabdomyosarcoma)), skin cancer (e.g., melanoma), thymus cancer (e.g., thymoma), or a combination thereof.

In some embodiments, the cancer is breast cancer (e.g., breast invasive carcinoma, breast invasive ductal carcinoma), central or peripheral nervous system tissue cancer (e.g., brain cancer (e.g., astrocytoma, glioblastoma, glioma, oligoastrocytoma)), endocrine or neuroendocrine cancer (e.g., adrenal cancer (e.g., adrenocortical carcinoma, pheochromocytoma, paraganglioma), thyroid cancer (e.g., papillary thyroid cancer)), eye cancer (e.g., uveal cancer (e.g., uveal melanoma)), gastrointestinal cancer (e.g., bile duct cancer (e.g., cholangiocarcinoma), colorectal cancer (e.g., colon adenocarcinoma, rectal adenocarcinoma, mucinous adenocarcinoma, mucinous carcinoma), esophageal cancer (e.g., esophageal adenocarcinoma), liver cancer (e.g., hepatocellular carcinoma), pancreatic cancer (e.g., pancreatic adenocarcinoma), or stomach cancer (e.g., stomach adenocarcinoma, signet ring cell carcinoma of the stomach)), genitourinary cancer (e.g., bladder cancer (e.g., bladder urothelial carcinoma), kidney cancer (e.g., renal clear cell carcinoma, renal papillary cell carcinoma, kidney chromophobe), prostate cancer (e.g., prostate adenocarcinoma), or testicular cancer (e.g., testicular germ cell tumors)), gynecologic cancer (e.g., cervical cancer (e.g., cervical squamous cell carcinoma, endocervical adenocarcinoma, mucinous carcinoma), ovarian cancer (e.g., serous ovarian cancer, ovarian serous cystadenocarcinoma), or uterine cancer (e.g., uterine carcinosarcoma, uterine endometrioid carcinoma, uterine serous carcinoma, uterine papillary serous carcinoma, uterine corpus endometrial carcinoma)), head and neck cancer (e.g., head and neck squamous cell carcinoma), hematological cancer (e.g., leukemia (e.g., chronic lymphocytic leukemia (CLL), acute lymphocytic leukemia (ALL) (e.g., B-cell lineage ALL (B-ALL), Philadelphia chromosome positive ALL (e.g., Philadelphia chromosome positive B-ALL), Philadelphia chromosome negative ALL (e.g., Philadelphia chromosome negative B-ALL)), acute myeloid leukemia (AML), chronic myeloid leukemia (CML)) or lymphoma (e.g., Hodgkin lymphoma (e.g., nodular lymphocyte predominant Hodgkin lymphoma (NLPHL)), non-Hodgkin lymphoma (e.g., Burkitt lymphoma (BL), diffuse large B-cell lymphoma (DLBCL), diffuse histiocytic lymphoma (DHL), follicular lymphoma (FL), intravascular large B-cell lymphoma (IVLBCL), mantle cell lymphoma (MCL), peripheral T-cell lymphoma (PTCL) (e.g., PTCL with a T follicular helper phenotype (PTPCL-TFH), angioimmunoblastic T-cell lymphoma (AITL), or PTCL not otherwise specified (PTCL-NOS)), small lymphocytic lymphoma (SLL)))), respiratory cancer (e.g., lung cancer (e.g., lung squamous cell carcinoma, lung adenocarcinoma, mesothelioma, non-small cell lung cancer (NSCLC))), sarcoma (e.g., leiomyosarcoma, myxofibrosarcoma), skin cancer (e.g., melanoma), thymus cancer (e.g., thymoma), or a combination thereof.

In some embodiments, the cancer is a hematological cancer (e.g., a lymphoma (e.g., diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), peripheral T-cell lymphoma (PTCL) (e.g., PTCL with a T follicular helper phenotype (PTPCL-TFH), angioimmunoblastic T-cell lymphoma (AITL), or PTCL not otherwise specified (PTCL-NOS)), small lymphocytic lymphoma (SLL), Burkitt lymphoma (BL), mantle cell lymphoma (MCL)) or a leukemia (e.g., chronic lymphocytic leukemia (CLL), acute lymphocytic leukemia (ALL) (e.g., B-cell lineage ALL (B-ALL), Philadelphia chromosome positive ALL (e.g., Philadelphia chromosome positive B-ALL), Philadelphia chromosome negative ALL (e.g., Philadelphia chromosome negative B-ALL)), chronic myeloid leukemia (CML))), breast cancer, gastrointestinal cancer, brain cancer (e.g., glioblastoma) or lung cancer (e.g., NSCLC). In some embodiments, the cancer is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), peripheral T-cell lymphoma (PTCL), chronic lymphocytic leukemia (CLL), acute lymphocytic leukemia (ALL), or chronic myeloid leukemia (CML). In some embodiments, the cancer is hematological cancer is DLBCL, FL, MCL, BL, PTCL, or ALL (e.g., B-ALL). In some embodiments, the cancer is FL or DLBCL. In some embodiments, the cancer is DLBCL, FL, MCL, or ALL (e.g., B-ALL). See, e.g., Leeman-Neill and Bhagat, Expert Opinion on Therapeutic Targets 22.2 (2018): 143-152; Mlynarczyk and Melnick. Immunological Reviews 288.1 (2019): 214-239; Hurtz, Christian, et al., Journal of Experimental Medicine 208.11 (2011): 2163-2174; Deb, Dhruba, et al. Cancer Research 77.11 (2017): 3070-3081; Cardenas, Mariano G., et al., Clinical Cancer Research 23.4 (2017): 885-893; Walker, Sarah R., et al., Oncogene 34.9 (2015): 1073-1082; International Publication Nos. WO 2021/080950, WO 2021/077010, and WO 2022/221673.

In some embodiments, the cancer is non-Hodgkin lymphoma (e.g., Burkitt lymphoma (BL), diffuse large B-cell lymphoma (DLBCL), diffuse histiocytic lymphoma (DHL), follicular lymphoma (FL), intravascular large B-cell lymphoma (IVLBCL), mantle cell lymphoma (MCL), peripheral T-cell lymphoma (PTCL) (e.g., PTCL with a T follicular helper phenotype (PTPCL-TFH)), or small lymphocytic lymphoma (SLL))). In some embodiments, the non-Hodgkin lymphoma is B-cell non-Hodgkin lymphoma. In some embodiments, the non-Hodgkin lymphoma is CD20-positive. In some embodiments, the non-Hodgkin lymphoma is CD20-positive B-cell non-Hodgkin lymphoma. In some embodiments, the patient has not been previously treated for the non-Hodgkin lymphoma. In some embodiments, the patient has previously received chemotherapy. In some embodiments, the patient has been previously treated with rituximab or obinutuzumab as a monotherapy or in combination with an additional therapy or therapeutic agent. In some embodiments, the patient has been previously treated with rituximab as a monotherapy or in combination with an additional therapy or therapeutic agent. In some embodiments, the patient has been previously treated with obinutuzumab as a monotherapy or in combination with an additional therapy or therapeutic agent. In some embodiments, the patient has previously been treated with R-CHOP (RITUXAN® (rituximab), cyclophosphamide, hydroxydaunorubicin, vincristine, and prednisone), or G-CHOP (GAZYVA® (obinutuzumab)), cyclophosphamide, hydroxydaunorubicin, vincristine, and prednisone). In some cases, the patient has previously been treated with etoposide and R-CHOP (called R-EPOCH). In some cases, the patient has been previously treated with R-CHOP combined with lenalidomide, venetoclax, ibrutinib, acalabrutinib, obinutuzumab, polatuzumab, pembrolizumab, durvalumab, or mosunetuzumab. In some embodiments, the patient has been previously treated with cyclophosphamide, vincristine, and prednisone (CVP), with or without rituximab or obinutuzumab. In some embodiments, the patient has received one or more lines of systemic therapy. In some embodiments, the patient has received two or more lines of systemic therapy. In some embodiments, the patient has previously been treated with a cell-based therapy (e.g., adoptive cell therapy (e.g., CAR T therapy, cytokine-induced killer cells (CIKs), natural killer cells (e.g., CAR-modified NK cells)) or antibody-armed cell therapy). In some embodiments, the non-Hodgkin lymphoma is non-progressing (including stable disease) non-Hodgkin lymphoma. In some embodiments, the non-Hodgkin lymphoma is relapsed or refractory non-Hodgkin lymphoma. In some embodiments, the patient is a patient who relapsed after, or is refractory to, a rituximab-containing regimen. In some embodiments, the patient is a patient who relapsed after, or is refractory to, an obinutuzumab-containing regimen. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, cancer is a non-Hodgkin lymphoma, and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is administered as a monotherapy.

In some embodiments, the cancer is DLBCL. In some embodiments, the DLBCL is characterized by a BCL2 translocation, a BCL6 translocation, a CD79B mutation (e.g., H225Y, A205D, Y196del, Y196F, Y196D, Y207X, Y196N, A205fs, Y196S, Y196H, A205fs, T206fs, H194_E197delinsQ, E197G, K219T, E192fs, or Y196C), an EZH2 mutation (e.g., a Y646F, Y646N, A682G, or A692V mutation), a MYC translocation, a MYD88 mutation (e.g., a L265P mutation), a NOTCHI mutation (e.g., Q2394X, Q2501X, Q2459X, Y2490X, G2427fs, Q2444X, P2514fs, or P2517S), a NOTCH2 mutation (e.g., Q2285K, S2136fs, Q2361X, P2288fs, L2415fs, G2410fs, Q2409X, S2388X, I2304fs, Q2364X, Q2360fs, S2395X, E2261fs, M2267fs, Q2285fs, R2400X, P2303fs, Q2285fs, A2273fs, K2133fs, Q2389X, E2399X, E2290X, Q2325X, Y2340X, Y2392X, or E2411fs), a TP53 mutation (e.g., R181C, E336A, R248W, P98fs, P152L, R280I, S149fs, P151H, G245D, Y236D, S127F, A161T, D148fs, M246I, Y126C, H179R, A159P, C₂₃₈G, L93fs, Y220C, R283fs, G244D, G245S, E171X, R209X, T155_R156dup, E271K, R306X, G105D, L93fs, G262V, W53X, G244V, H214Y, R282W, R337C, Q331fs, R273G, R273C, C176Y, S215R, R213Q, I195T, G245R, I232T, R175H, Y126D, R273H, R196X, Y205C, C141Y, C229X, Y126N, P278S, P151S, Y236H, R282G, Y103X, V216M, G244S, G266E, V173A, V173fs, I254S, T125M, R342X, P152fs, Y205D, V274L, L257P, C135Y, C176R, Y234N, R248Q, G244R, Y234H, R248G, M237I, R213X, E258D, V173M, L252_1254del, L252I, Y234C, or C176F), 17p deletion, 18q gain, or a combination thereof. In some embodiments, the DLBCL has a BCL6 rearrangement, a NOTCH2 mutation (e.g., Q2285K, S2136fs, Q2361X, P2288fs, L2415fs, G2410fs, Q2409X, S2388X, I2304fs, Q2364X, Q2360fs, S2395X, E2261fs, M2267fs, Q2285fs, R2400X, P2303fs, Q2285fs, A2273fs, K2133fs, Q2389X, E2399X, E2290X, Q2325X, Y2340X, Y2392X, E2411fs), or a combination thereof. In some embodiments, the DLBCL is DLBCL having a germinal center B cell (GCB) cell of origin. In some embodiments, the DLBCL is a BN2-type DLBCL (e.g., having a BCL6 rearrangement and/or a NOTCH2 mutation). In some embodiments, the DLBCL is an EZB-type DLBCL (e.g., having an EZH2 mutation and/or a BCL2 translocation). In some embodiments, the DLBCL is a C1 genetic cluster DLBCL (e.g., having a BCL6 rearrangement and/or a NOTCH2 mutation). See, e.g., Schmitz, Roland, et al. New England Journal of Medicine 378.15 (2018): 1396-1407; Chapuy, Bjoern, et al. Nature Medicine 24.5 (2018): 679-690 for additional description of these classifications.

In some embodiments, the cancer is a FL. In some embodiments, the FL has a BCL2 translocation (e.g., a t (14;18) translocation). In some embodiments, the FL has an EZH2 mutation (e.g., a Y646F, Y646N, A682G, or A692V mutation). See, e.g., Kridel, Robert, Laurie H. Sehn, and Randy D. Gascoyne. The Journal of Clinical Investigation 122.10 (2012): 3424-3431. In some embodiments, the cancer is a FL, and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is administered as a monotherapy.

In some embodiments, the cancer is a B-ALL. In some embodiments, the B-ALL has an MLL rearrangement (e.g., an MLL-Af4 fusion, an MLL-Af6 fusion, an MLL-Af9 fusion, an MLL-ENL fusion, or an MLL-PTD fusion), is pre-B cell receptor positive (Pre-BCR+), has the Philadelphia chromosome, is Philadelphia chromosome-like, is dependent on Ras signaling, has a BCL2 amplification, has a JAK2 mutation (with or without high cytokine receptor-like factor 2 (CRLF2) expression), or a combination thereof. See, e.g., Knight, Thomas, and Julie Anne Elizabeth Irving. Frontiers in Oncology 4 (2014): 160; Geng, Huimin, et al. Cancer Cell 27.3 (2015): 409-425; Jain, Nitin, et al. Blood, 129.5 (2017): 572-581; and Hurtz, Christian, et al. Genes & Development 33.17-18 (2019): 1265-1279. In some embodiments, the cancer is a B-ALL, and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is administered as a monotherapy. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is used in the treatment of patient having a B-ALL. In some embodiments, the B-ALL is a relapsed or refractory B-ALL after two or more lines of systemic therapy.

In some embodiments, the patient has previously been treated with another anticancer agent, a chemotherapeutic agent, surgery, radiation, a multi-kinase inhibitor, or a combination thereof.

In some embodiments, the cancer is a DLBCL. In some embodiments, the patient has not been previously treated for the DLBCL. In some embodiments, the patient has previously received chemotherapy. In some embodiments, the patient has been previously treated with rituximab or obinutuzumab as a monotherapy or in combination with an additional therapy or therapeutic agent. In some embodiments, the patient has been previously treated with rituximab as a monotherapy or in combination with an additional therapy or therapeutic agent. In some embodiments, the patient has been previously treated with obinutuzumab as a monotherapy or in combination with an additional therapy or therapeutic agent. In some embodiments, the patient has previously been treated with lenalidomide in combination with rituximab or obinutuzumab. In some embodiments, the patient has previously been treated with cyclophosphamide, vincristine and prednisone (CVP), optionally in combination with rituximab or obinutuzumab. In some embodiments, the patient has previously been treated with R-CHOP (RITUXAN® (rituximab), cyclophosphamide, hydroxydaunorubicin, vincristine, and prednisone), or G-CHOP (GAZYVAR (obinutuzumab)), cyclophosphamide, hydroxydaunorubicin, vincristine, and prednisone). In some cases, the patient has previously been treated with etoposide and R-CHOP (called R-EPOCH). In some cases, the patient has been previously treated with R-CHOP combined with lenalidomide, venetoclax, ibrutinib, acalabrutinib, obinutuzumab, polatuzumab, pembrolizumab, durvalumab, or mosunetuzumab. In some embodiments, the patient has been previously treated with a rituximab-containing regimen. In some embodiments, the patient has been previously treated with an obinutuzumab-containing regimen. In some embodiments, the patient has been previously treated with a mosunetuzumab-containing regimen. In some embodiments, the patient has been previously treated with an epcoritamab-containing regimen. In some embodiments, the patient has received one or more lines of systemic therapy. In some embodiments, the patient has received two or more lines of systemic therapy. In some embodiments, the patient has previously been treated with a cell-based therapy (e.g., adoptive cell therapy (e.g., CAR T therapy, cytokine-induced killer cells (CIKs), natural killer cells (e.g., CAR-modified NK cells)) or antibody-armed cell therapy). In some embodiments, the DLBCL is non-progressing (including stable disease) DLBCL. In some embodiments, the DLBCL is relapsed or refractory DLBCL. In some embodiments, the patient is a patient who relapsed after, or is refractory to, a rituximab-containing regimen. In some embodiments, the patient is a patient who relapsed after, or is refractory to, an obinutuzumab-containing regimen. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, the cancer is a DLBCL, and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is administered as a monotherapy. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is used in the treatment of patient having a DLBCL. In some embodiments, the DLBCL is a relapsed or refractory DLBCL after two or more lines of systemic therapy.

In some embodiments, the cancer is a FL. In some embodiments, the patient has not been previously treated for the FL. In some embodiments, the patient has previously received chemotherapy. In some embodiments, the patient has been previously treated with rituximab or obinutuzumab as a monotherapy or in combination with an additional therapy or therapeutic agent. In some embodiments, the patient has been previously treated with rituximab as a monotherapy or in combination with an additional therapy or therapeutic agent. In some embodiments, the patient has been previously treated with obinutuzumab as a monotherapy or in combination with an additional therapy or therapeutic agent. In some embodiments, the patient has previously been treated with rituximab or obinutuzumab monotherapy. In some embodiments, the patient has previously been treated with bendamustine in combination with rituximab or obinutuzumab. In some embodiments, the patient has previously been treated with lenalidomide in combination with rituximab or obinutuzumab (the combination with rituximab is sometimes called “R²”). In some embodiments, the patient has previously been treated with cyclophosphamide, vincristine and prednisone (CVP), optionally in combination with rituximab or obinutuzumab. In some embodiments, the patient has previously been treated with R-CHOP or G-CHOP. In some embodiments, the patient has received one or more lines of systemic therapy. In some embodiments, the patient has received two or more lines of systemic therapy. In some embodiments, the patient has previously been treated with a cell-based therapy (e.g., adoptive cell therapy (e.g., CAR T therapy, cytokine-induced killer cells (CIKs), natural killer cells (e.g., CAR-modified NK cells)) or antibody-armed cell therapy). In some embodiments, the non-Hodgkin lymphoma is non-progressing (including stable disease) FL. In some embodiments, the FL is relapsed or refractory FL. In some embodiments, the patient is a patient who relapsed after, or is refractory to, a rituximab-containing regimen. In some embodiments, the patient is a patient who relapsed after, or is refractory to, an obinutuzumab-containing regimen. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, the cancer is a FL, and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is administered as a monotherapy. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is used in the treatment of patient having a FL. In some embodiments, the FL is a relapsed or refractory FL after two or more lines of systemic therapy.

In some embodiments, the cancer is a BL. In some embodiments, the patient has not been previously treated for the BL. In some embodiments, the patient has previously received chemotherapy. In some embodiments, the patient has been previously treated with rituximab or obinutuzumab as a monotherapy or in combination with an additional therapy or therapeutic agent. In some embodiments, the patient has been previously treated with rituximab as a monotherapy or in combination with an additional therapy or therapeutic agent. In some embodiments, the patient has been previously treated with obinutuzumab as a monotherapy or in combination with an additional therapy or therapeutic agent. In some embodiments, the patient has previously been treated with rituximab or obinutuzumab monotherapy. In some embodiments, the patient has previously been treated with bendamustine in combination with rituximab or obinutuzumab. In some embodiments, the patient has previously been treated with R-CHOP or G-CHOP. In some embodiments, the patient has previously been treated with rituximab, cyclophosphamide, vincristine, doxorubicin, and methotrexate (R-CODOX-M). In some embodiments, the patient has previously been treated with rituximab, ifosfamide, etoposide, and cytarabine (R-IVAC). In some embodiments, the patient has previously been treated with rituximab with dose-adjusted etoposide, prednisolone, vincristine, cyclophosphamide, and doxorubicin (DA-EPOCH-R). In some embodiments, the patient has received one or more lines of systemic therapy. In some embodiments, the patient has received two or more lines of systemic therapy. In some embodiments, the patient has previously been treated with a cell-based therapy (e.g., adoptive cell therapy (e.g., CAR T therapy, cytokine-induced killer cells (CIKs), natural killer cells (e.g., CAR-modified NK cells)) or antibody-armed cell therapy). In some embodiments, the BL is non-progressing (including stable disease) BL. In some embodiments, the BL is relapsed or refractory BL. In some embodiments, the patient is a patient who relapsed after, or is refractory to, a rituximab-containing regimen. In some embodiments, the patient is a patient who relapsed after, or is refractory to, an obinutuzumab-containing regimen. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, the cancer is a BL, and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is administered as a monotherapy. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is used in the treatment of patient having a BL. In some embodiments, the BL is a relapsed or refractory BL after two or more lines of systemic therapy.

In some embodiments, the cancer is a PTCL (e.g., PTCL with a T follicular helper phenotype (PTPCL-TFH), angioimmunoblastic T-cell lymphoma (AITL), or PTCL not otherwise specified (PTCL-NOS)). In some embodiments, the patient has not been previously treated for the PTCL. In some embodiments, the patient has previously received chemotherapy. In some embodiments, the patient has been previously treated with rituximab or obinutuzumab as a monotherapy or in combination with an additional therapy or therapeutic agent. In some embodiments, the patient has been previously treated with rituximab as a monotherapy or in combination with an additional therapy or therapeutic agent. In some embodiments, the patient has been previously treated with obinutuzumab as a monotherapy or in combination with an additional therapy or therapeutic agent. In some embodiments, the patient has previously been treated with rituximab or obinutuzumab monotherapy. In some embodiments, the patient has previously been treated with bendamustine in combination with rituximab or obinutuzumab. In some embodiments, the patient has previously been treated with lenalidomide in combination with rituximab or obinutuzumab (the combination with rituximab is sometimes called “R²”). In some embodiments, the patient has previously been treated with cyclophosphamide, vincristine and prednisone (CVP), optionally in combination with rituximab or obinutuzumab (R-CVP or G-CVP, respectively). In some embodiments, the patient has previously been treated with R-CHOP or G-CHOP. In some embodiments, the patient has received one or more lines of systemic therapy. In some embodiments, the patient has received two or more lines of systemic therapy. In some embodiments, the patient has previously been treated with a cell-based therapy (e.g., adoptive cell therapy (e.g., CAR T therapy, cytokine-induced killer cells (CIKs), natural killer cells (e.g., CAR-modified NK cells)) or antibody-armed cell therapy). In some embodiments, the PTCL is non-progressing (including stable disease) PTCL. In some embodiments, the PTCL is relapsed or refractory PTCL. In some embodiments, the patient is a patient who relapsed after, or is refractory to, a rituximab-containing regimen. In some embodiments, the patient is a patient who relapsed after, or is refractory to, an obinutuzumab-containing regimen. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, the cancer is a PTCL, and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is administered as a monotherapy. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is used in the treatment of a patient having a PTCL. In some embodiments, the PTCL is a relapsed or refractory PTCL after two or more lines of systemic therapy.

In some embodiments, the cancer is B-ALL. In some embodiments, the B-ALL is Philadelphia chromosome positive B-ALL. In some embodiments, the B-ALL is Philadelphia chromosome negative B-ALL. In some embodiments, the patient has previously received chemotherapy. In some embodiments, the patient has previously been treated with at least one cycle of induction, consolidation, intensification, and optional maintenance. In some cases, induction therapy can include an anthracycline, vincristine, a corticosteroid, and cyclophosphamide. In some embodiments, the anthracycline is doxorubicin. In some embodiments, the corticosteroid is dexamethasone. In some cases, the combination of doxorubicin, vincristine, dexamethasone, and cyclophosphamide is known as CVAD. In some embodiments, induction therapy can further include a tyrosine kinase inhibitor (e.g., a BCR-ABL inhibitor for patients with this fusion). In some embodiments, induction therapy can further include asparaginase (e.g., for pediatric patients). In some cases, consolidation therapy can include methotrexate, cytarabine, vincristine, 6-mercaptopurine, 6-thioguanine, cyclophosphamide, and etoposide. In some embodiments, consolidation therapy can further include a tyrosine kinase inhibitor (e.g., a BCR-ABL inhibitor for patients with this fusion). In some embodiments, consolidation therapy can further include asparaginase (e.g., for pediatric patients). In some cases, intensification therapy can include an anthracycline, vincristine, a corticosteroid, and cyclophosphamide. In some embodiments, intensification therapy can further include a tyrosine kinase inhibitor (e.g., a BCR-ABL inhibitor for patients with this fusion). In some embodiments, intensification therapy can further include asparaginase (e.g., for pediatric patients). Typically, pediatric and young adult regimens include higher cumulative doses of asparaginase and vincristine but may have lower cumulative doses of anthracycline and cyclophosphamide compared to adult regimens. In any of these cycle phases, an anti-CD20 immunotherapy (e.g., rituximab) can be added for patients expressing the CD20 protein on the cells. See, e.g., Muffly, Lori, and Emily Curran. Hematology 2014, the American Society of Hematology Education Program Book 2019.1 (2019): 17-23. In some embodiments, the patient has received one or more lines of systemic therapy. In some embodiments, the patient has received two or more lines of systemic therapy. In some embodiments, the B-ALL is relapsed or refractory B-ALL. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, the cancer is a B-ALL, and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is administered as a monotherapy. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is used in the treatment of patient having a B-ALL. In some embodiments, the B-ALL is a relapsed or refractory B-ALL after two or more lines of systemic therapy.

Also provided herein is a method of treating a subject having a cancer, wherein the method comprises:
administering a therapeutically effective amount of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, as a monotherapy or in conjunction with a first anticancer agent to the subject who has been administered one or more doses of the first anticancer agent to the subject for a period of time.

Also provided herein is a method of treating a subject having a cancer, wherein the method comprises:
(a) administering one or more doses of a first anticancer agent to the subject for a period of time; and
(b) after (a), administering a therapeutically effective amount of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, as a monotherapy or in conjunction with the first anticancer agent to the subject.

Also provided herein is a method of treating a subject having a cancer, wherein the method comprises:
(a) administering one or more doses of a first anticancer agent to the subject for a period of time; and
(b) after (a), administering a therapeutically effective amount of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, as a monotherapy or in conjunction with a second anticancer agent to the subject.

In some embodiments of any of the methods of treating cancers provided herein, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is dosed q.d. (once daily) to the subject. In some embodiments of any of the methods of treating cancers provided herein, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is dosed b.i.d. (twice daily) to the subject. In some embodiments of any of the methods of treating cancers provided herein, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is dosed t.i.d. (three times daily) to the subject. In some embodiments of any of the methods of treating cancers provided herein, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is dosed q.i.d. (four times daily) to the subject. In some embodiments of any of the methods of treating cancers provided herein, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is dosed q.o.d. (every other day) to the subject. In some embodiments of any of the methods of treating cancers provided herein, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is dosed q.week (once weekly) to the subject. In some embodiments of any of the methods of treating cancers provided herein, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is dosed b.i.w. (twice weekly) to the subject. In some embodiments of any of the methods of treating cancers provided herein, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is dosed t.i.w. (three times weekly) to the subject.

BCL6 activity has also been implicated in autoimmunity. See, for example, Li, Qing, et al. European Journal of Immunology 50.4 (2020): 525-536; Pearce, Andrew C., et al. Journal of Biological Chemistry 297.2 (2021); Venkatadri, Rajkumar, et al. European Journal of Immunology 52.5 (2022): 825-834; Patel, Preeyam S., et al. Science Advances 8.25 (2022): eabo1782; Ding, Shu, Yu Rao, and Qianjin Lu. Cellular & Molecular Immunology 19.7 (2022): 863-865. Accordingly, also provided herein is a method of treating an autoimmune condition in a subject in need thereof, the method comprising administering to the subject a therapeutically effective amount of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition comprising a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof.

Provided herein is use of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition thereof, for the treatment of an autoimmune condition, for example, any of the autoimmune conditions provided herein.

Provided herein is use of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition thereof, as a medicament for the treatment of an autoimmune condition, for example, any of the autoimmune conditions provided herein.

Provided herein is use of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, in the manufacture of a medicament for the treatment of an autoimmune condition, for example, any of the autoimmune conditions provided herein.

Provided herein is a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition thereof, for use as a medicament for the treatment of an autoimmune condition, for example, any of the autoimmune conditions provided herein.

Provided herein is a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition thereof, for use in treating an autoimmune condition, for example, any of the autoimmune conditions provided herein.

In some embodiments, the autoimmune condition is acquired hemophilia, Addison's disease, ankylosing spondylitis, anti-neutrophil cytoplasmic antibody associated vasculitis (ANCA vasculitis), anti-synthetase syndrome, atherosclerosis, autoimmune hemolytic anemia, autoimmune hepatitis, autoimmune sclerosing cholangitis, autoimmune thyroiditis, autoimmune uveitis, Crohn's disease, dermatomyositis, diffuse scleroderma, Goodpasture's syndrome, graft-versus-host disease (GVHD) (e.g., chronic graft-versus-host disease (cGVHD)), Graves' disease, Guillain-Barre syndrome, Hashimoto's thyroiditis, Hughes' syndrome, IgG4-related disease, immune thrombocytopeniarpura (ITP), inflammatory bowel disease, limited scleroderma, multiple sclerosis, myasthenia gravis (MG), neuromyelitis optica spectrum disorders (NMOSD) (e.g., neuromyelitis optica (NMO)), pemphigoid, pemphigus, pernicious anemia, polymyositis, primary biliary cirrhosis, psoriasis, psoriatic arthritis, rheumatoid arthritis, seronegative spondyloarthropathies, Sjogren's syndrome, systemic lupus erythematosus, thrombocytopenia purpura, Type 1 diabetes, ulcerative colitis, vitiligo, or a combination thereof. In some embodiments, the autoimmune condition is rheumatoid arthritis, systemic lupus erythematosus, or a combination thereof. In some embodiments, the autoimmune condition is ANCA vasculitis, GVHD (e.g., cGVHD), myasthenia gravis, NMO, or a combination thereof. In some embodiments, the autoimmune condition is ANCA vasculitis, anti-synthetase syndrome, arthritis (e.g., rheumatoid arthritis or inflammatory arthritis), GVHD (e.g., cGVHD), IgG4-RD, lupus (e.g., lupus erythematosus), ITP, MG (e.g., muscle-specific tyrosine kinase (MuSK) positive MG), MS, NMOSD (e.g., NMO), pemphigus (e.g., pemphigus vulgaris), Sjogren's syndrome, or a combination thereof. In some embodiments, the autoimmune condition is ANCA vasculitis, anti-synthetase syndrome, GVHD (e.g., cGVHD), TIP, MG (e.g., muscle-specific tyrosine kinase (MuSK) positive MG), NMOSD (e.g., NMO), pemphigus, or a combination thereof. In some embodiments, the autoimmune condition is arthritis (e.g., rheumatoid arthritis or inflammatory arthritis), GVHD (e.g., cGVHD), IgG4-RD, lupus (e.g., lupus erythematosus), MG (e.g., muscle-specific tyrosine kinase (MuSK) positive MG), MS, NMOSD (e.g., NMO), pemphigus (e.g., pemphigus vulgaris), Sjogren's syndrome, or a combination thereof. See, e.g., Pearce, Andrew C., et al. Journal of Biological Chemistry 297.2 (2021); Ding, Shu, Yu Rao, and Qianjin Lu, Cellular & Molecular Immunology (2022): 1-3; Lee, Dennis S W, Olga L. Rojas, and Jennifer L. Gommerman, Nature Reviews Drug Discovery 20.3 (2021): 179-199; and International Publication Nos. WO 2020/014599; WO 2021/074620.

In some embodiments of any of the methods of treating autoimmune conditions provided herein, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is dosed q.d. (once daily) to the subject. In some embodiments of any of the methods of treating autoimmune conditions provided herein, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is dosed b.i.d. (twice daily) to the subject. In some embodiments of any of the methods of treating autoimmune conditions provided herein, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is dosed t.i.d. (three times daily) to the subject. In some embodiments of any of the methods of treating autoimmune conditions provided herein, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is dosed q.i.d. (four times daily) to the subject. In some embodiments of any of the methods of treating autoimmune conditions provided herein, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is dosed q.o.d. (every other day) to the subject. In some embodiments of any of the methods of treating autoimmune conditions provided herein, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is dosed q.week (once weekly) to the subject. In some embodiments of any of the methods of treating autoimmune conditions provided herein, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is dosed b.i.w. (twice weekly) to the subject. In some embodiments of any of the methods of treating autoimmune conditions provided herein, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is dosed t.i.w. (three times weekly) to the subject.

Also provided herein is a method of treating a lymphoproliferative disorder in a subject in need thereof, the method comprising administering to the subject a therapeutically effective amount of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition comprising a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof.

Provided herein is use of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition thereof, for the treatment of a lymphoproliferative disorder, for example, any of the lymphoproliferative disorders provided herein.

Provided herein is use of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition thereof, as a medicament for the treatment of a lymphoproliferative disorder, for example, any of the lymphoproliferative disorders provided herein.

Provided herein is use of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, in the manufacture of a medicament for the treatment of a lymphoproliferative disorder, for example, any of the lymphoproliferative disorders provided herein.

Provided herein is a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition thereof, for use as a medicament for the treatment of a lymphoproliferative disorder, for example, any of the lymphoproliferative disorders provided herein.

Provided herein is a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition thereof, for use in treating a lymphoproliferative disorder, for example, any of the lymphoproliferative disorders provided herein.

In some embodiments, the lymphoproliferative disorder is Epstein-Barr Virus-associated lymphoproliferative disorder.

Also provided is a method for modulating (e.g., decreasing) BCL6 protein activity in a cell, comprising contacting the cell with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, to a subject. In some embodiments, the cell is a cancer cell. In some embodiments, the cell is a mammalian cell. In some embodiments, the cell is a mammalian cancer cell. In some embodiments, the cancer cell is any cancer as described herein. As used herein, the term “contacting” refers to the bringing together of indicated moieties in an in vitro system or an in vivo system. For example, “contacting” a cell with a compound provided herein includes the administration of a compound provided herein to the cell, in vitro or in vivo, including, for example, introducing a compound provided herein into a sample containing cells (e.g., grown in culture or derived from a patient), an organoid, or an organism (e.g., an animal (e.g., an animal bearing a tumor), or a human).

Also provided is a method of modulating (e.g., decreasing) the level of BCL6 protein in a cell, comprising contacting the cell with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof. In some embodiments, the level of BCL6 protein is decreased by at least 30% (e.g., at least 40%, at least 50%, at least 60%, at least 70%, at least 80%, at least 90%, at least 95%, at least 97%, or at least 99%) compared to a cell not contacted with the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, to a subject having a cell having a BCL6 protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided is a method of inducing ubiquitination of a BCL6 protein in a cell, comprising contacting the cell with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, to a subject having a cell having a BCL6 protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided is a method of forming a ternary complex comprising a BCL6 protein, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, and a CRBN protein or fragment thereof in a cell, comprising contacting the cell with a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, to a subject having a cell having a BCL6 protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided herein is a method for inducing degradation of a BCL6 protein in a mammalian cell, the method comprising contacting the mammalian cell with an effective amount of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof.

Also provided herein is a method of inhibiting cell proliferation, in vitro or in vivo, the method comprising contacting a cell with an effective amount of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition thereof as defined herein.

Further provided herein is a method of increasing cell death, in vitro or in vivo, the method comprising contacting a cell with an effective amount of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or a pharmaceutical composition thereof as defined herein. Also provided herein is a method of increasing tumor cell death in a subject. The method comprises administering to the subject a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, in an amount effective to increase tumor cell death.

When employed as pharmaceuticals, the compounds of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or pharmaceutically acceptable salts thereof, can be administered in the form of pharmaceutical compositions as described herein.

### Combinations

In any of the indications described herein, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can be used as a monotherapy. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, can be used prior to administration of an additional therapeutic agent or additional therapy. For example, a subject in need thereof can be administered one or more doses of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, for a period of time and then undergo at least partial resection of the tumor. In some embodiments, the treatment with one or more doses of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, reduces the size of the tumor (e.g., the tumor burden) prior to the at least partial resection of the tumor.

In some embodiments, a subject in need thereof can be administered one or more doses of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, for a period of time and undergo one or more rounds of radiation therapy. In some embodiments, the treatment with one or more doses of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, reduces the size of the tumor (e.g., the tumor burden) prior to the one or more rounds of radiation therapy.

In some embodiments of any the methods described herein, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is administered in combination with a therapeutically effective amount of at least one additional therapeutic (e.g., chemotherapeutic) agent.

Non-limiting examples of additional therapeutic agents include: RAS pathway targeted therapeutic agents (e.g., Ras/RAF/MEK/PI3K pathway inhibitors, (e.g., Ras inhibitors, KRas-targeted therapeutic agents, SOS1 inhibitors, SOS1/Ras protein-protein interaction inhibitors, SHP2 inhibitors, PI3K-AKT-mTOR pathway inhibitors)), kinase-targeted therapeutics (e.g., MEK inhibitors, ERK inhibitors, Raf inhibitors (e.g., BRaf inhibitors), PI3K inhibitors, AKT inhibitors, BTK inhibitors, mTOR inhibitors, CDK4/5 inhibitors, CDK4/6 inhibitors, MET inhibitors, JAK inhibitors (e.g., JAK2 inhibitors), FAK inhibitors, ErbB family inhibitors (e.g., EGFR inhibitors, Her2 inhibitors), Src inhibitors), menin inhibitors, mTORC1 inhibitors, YAP inhibitors, proteasome inhibitors, farnesyl transferase inhibitors, HSP90 inhibitors, PTEN inhibitors, inhibitors of the polycomb repressive complex 2 (PRC2) (e.g., EZH1/2 or EZH2 inhibitors), signal transduction pathway inhibitors, checkpoint inhibitors, modulators of the apoptosis pathway (e.g., BCL-2 inhibitors, BCL-XL inhibitors), XPO1 inhibitors, steroids, chemotherapeutics, angiogenesis-targeted therapies, immune-targeted agents including immunomodulatory imide drugs (sometimes called “IMiDs” or “CELMoDs”), immunotherapy (e.g., anti-PD1, anti-PD-L1, anti-CD19, anti-CD20, anti-CD22, anti-CD3, anti-CD30, anti-CD79B, or anti-CD47 therapies, including antibodies (e.g., single-targeted antibodies targeting one or more of PD1, PD-L1, CD19, CD20, CD22, CD3, CD30, CD79B, or CD47, bispecific antibodies (including bispecific T cell engagers (BiTEs)) targeting one or more of PD1, PD-L1, CD19, CD20, CD22, CD3, CD30, CD79B, or CD47, and antibody-drug conjugates (ADCs) incorporating one or more of PD1, PD-L1, CD19, CD20, CD22, CD3, CD30, CD79B, or CD47 antibodies) or antigen-binding fragments thereof, a PD-1 inhibitor, or a PD-L1 inhibitor), cell-based therapeutics (e.g., adoptive cell therapy (e.g., CAR T therapy, cytokine-induced killer cells (CIKs), natural killer cells (e.g., CAR-modified NK cells)) or antibody-armed cell therapy), and radiotherapy.

As used herein, a biosimilar antibody refers to an antibody or antigen-binding fragment that has the same primary amino acid sequence as compared to a reference antibody and optionally, may have detectable differences in post-translation modifications (e.g., glycosylation and/or phosphorylation) as compared to the reference antibody (e.g., a different glycoform).

In some embodiments, the additional therapeutic agent is a PI3K inhibitor, an Abl inhibitor (e.g., a BCR-Abl inhibitor), a BTK inhibitor, a JAK inhibitor (e.g., a JAK2 inhibitor), a BRaf inhibitor, a MEK inhibitor, a menin inhibitor, a BCL-2 inhibitor, a BCL-XL inhibitor, an MCL-1 inhibitor, an XPO1 inhibitor, an inhibitor of the polycomb repressive complex 2 (e.g., an EZH1/2 or EZH2 inhibitor), an immunomodulatory imide drug, a steroid, anti-CD19 therapy, anti-CD20 therapy, anti-CD3 therapy, chemotherapy, or a combination thereof.

Without being bound by any particular theory, it is believed that targeting BCL6 can result in the induction of genes that it typically represses, such as BCL2. In some embodiments, the additional therapeutic agent is a BCL-2 inhibitor (e.g., venetoclax, navitoclax, lisaftoclax, obatoclax, pelcitoclax, AZD-0466, BGB-11417, UBX-1325, UBX-1967, ZN-d5, oblimersen (e.g., oblimersen sodium), or beclanorsen).

In some embodiments, the PI3K inhibitor is alpelisib (BYL719), amdizalisib, apitolisib (GDC-0980), bimiralisib, buparlisib (BKM120), copanlisib (ALIQOPA™, BAY80-6946) (e.g., copanlisib dihydrochloride or a hydrate of copanlisib dihydrochloride), dactolisib (NVP-BEZ235, BEZ-235), dezapelisib, dordaviprone, duvelisib (e.g., a hydrate of duvelisib), eganelisib, fimepinostat, gedatolisib (PF-05212384, PKI-587), idelalisib, inavolisib, leniolisib (e.g., leniolisib phosphate), linperlisib, omipalisib (GSK2126458, GSK458), parsaclisib, pictilisib (GDC-0941), pilaralisib (XL147, SAR245408), paxalisib, rigosertib, risovalisib, seletalisib, serabelisib (TAK-117, MLN1117, INK 1117), sonolisib (PX-866), taselisib (GDC-0032, RG7604), umbralisib (e.g., umbralisib tosylate), voxtalisib (XL756, SAR245409), wortmannin, zandelisib, AMG 511, AMG319, ASN003, AZD8835, BGT-226 (NVP-BGT226), CH5132799, CUDC-907, GDC-0077, GDC-0084 (RG7666), GS-9820, GSK1059615, GSK2636771, KIN-193 (AZD-6428), LY2023414, LY294002, PF-04691502, PI-103, PKI-402, PQR309, SAR260301, SF1126, SHC-014748-M, TQ-B-3525, VS-5584 (SB2343), WX-037, XL-765, ZSTK474, or a combination thereof. In some embodiments, the PI3K inhibitor is alpelisib, amdizalisib, apitolisib, bimiralisib, buparlisib, copanlisib (e.g., copanlisib dihydrochloride or a hydrate of copanlisib dihydrochloride), dactolisib, dezapelisib, dordaviprone, duvelisib (e.g., a hydrate of duvelisib), eganelisib, fimepinostat, gedatolisib, idelalisib, inavolisib, leniolisib (e.g., leniolisib phosphate), linperlisib, parsaclisib, paxalisib, risovalisib, seletalisib, serabelisib, sonolisib, tenalisib, umbralisib (e.g., umbralisib tosylate), zandelisib, PF-04691502, SHC-014748-M, TQ-B-3525, or a combination thereof.

In some embodiments, the Abl inhibitor (e.g., BCR-Abl inhibitor) is asciminib (e.g., asciminib hydrochloride), bafetinib, bosutinib (e.g., bosutinib monohydrate), danusertib, dasatinib (e.g., dasatinib monohydrate), flumatinib (e.g., flumatinib mesylate), imatinib (e.g., imatinib mesylate), nilotinib (e.g., nilotinib monochloride monohydrate), olverembatinib (e.g., olverembatinib mesylate), ponatinib (e.g., ponatinib hydrochloride), radotinib (e.g., radotinib dihydrochloride), ruserontinib, vandetanib, AN-019, AT-9283, IKT-148009, NPB-001-056, or a combination thereof.

In some embodiments, the cancer is a B-ALL, and the additional therapy or therapeutic agent is an Abl inhibitor. In some embodiments, the cancer is a Philadelphia chromosome positive B-ALL and the additional therapy or therapeutic agent is an Abl inhibitor. In some embodiments, the cancer is a Philadelphia chromosome-like B-ALL, and the additional therapy or therapeutic agent is an Abl inhibitor. In some embodiments, the Abl inhibitor is selected from the group consisting of imatinib, dasatinib, ponatinib, or a combination thereof.

In some embodiments, the BTK inhibitor is abivertinib, acalabrutinib, atuzabrutinib, branebrutinib, dasatinib (e.g., dasatinib monohydrate), edralbrutinib (SHR-1459), elsubrutinib, evobrutinib, fenebrutinib, ibrutinib, luxeptinib, nemtabrutinib, olafertinib, nemtabrutinib, orelabrutinib, pirtobrutinib, remibrutinib, rilzabrutinib, spebrutinib, sunvozertinib, tirabrutinib (e.g., tirabrutinib hydrochloride), tolebrutinib, vecabrutinib, zanubrutinib, AC-0058 (AC-0058TA), BMS-986142, CT-1530, DTRMWXHS-12, LY-3337641 (HM-71224), M-7583, TAS-5315, or a combination thereof. In some embodiments, the BTK inhibitor is abivertinib, acalabrutinib, atuzabrutinib, branebrutinib, dasatinib (e.g., dasatinib monohydrate), edralbrutinib, elsubrutinib, evobrutinib, fenebrutinib, ibrutinib, nemtabrutinib, orelabrutinib, pirtobrutinib, remibrutinib, rilzabrutinib, sunvozertinib, tirabrutinib (e.g., tirabrutinib hydrochloride), tolebrutinib, zanubrutinib, AC-0058, BMS-986142, DTRMWXHS-12, LY-3337641, TAS-5315, or a combination thereof.

In some embodiments, the cancer is a B-ALL, and the additional therapy or therapeutic agent is a BTK inhibitor. In some embodiments, the cancer is a pre-BCR+B-ALL, and the additional therapy or therapeutic agent is a BTK inhibitor. In some embodiments, the cancer is a B-ALL dependent on Ras signaling, and the additional therapy or therapeutic agent is a BTK inhibitor. In some embodiments, the BTK inhibitor is ibrutinib or acalabrutinib.

In some embodiments, the JAK inhibitor is abrocitinib, baricitinib, brepocitinib, decernotinib, delgocitinib, deuruxolitinib, elsubrutinib, fedratinib (e.g., fedratinib dihydrochloride monohydrate), filgotinib (e.g., filgotinib maleate), gandotinib, gusacitinib, ilginatinib, itacitinib, ivarmacitinib, izencitinib, jaktinib, momelotinib, nezulcitinib, pacritinib (e.g., pacritinib citrate), peficitinib (e.g., peficitinib hydrobromide), povorcitinib (INCB-54707), ropsacitinib, ruxolitinib (e.g., ruxolitinib phosphate), solcitinib, tasocitinib (e.g., tofacitinib citrate), tinengotinib, upadacitinib (e.g., upadacitinib hydrate), zasocitinib, AGA-201, ATI-1777, ATI-501, ESK-001, GLPG-3667, INCB-52793, LNK-01001, LNK-01003, R-348, TD-8236, TLL-018, TQ-05105, VTX-958, or a combination thereof. In some embodiments, the JAK inhibitor is abrocitinib, baricitinib, brepocitinib, decernotinib, delgocitinib, deuruxolitinib, fedratinib (e.g., fedratinib dihydrochloride monohydrate), filgotinib (e.g., filgotinib maleate), gandotinib, gusacitinib, ilginatinib, itacitinib, ivarmacitinib, izencitinib, jaktinib, momelotinib, nezulcitinib, pacritinib (e.g., pacritinib citrate), peficitinib (e.g., peficitinib hydrobromide), povorcitinib (INCB-54707), ropsacitinib, ruxolitinib (e.g., ruxolitinib phosphate), solcitinib, tasocitinib (e.g., tofacitinib citrate), tinengotinib, upadacitinib (e.g., upadacitinib hydrate), zasocitinib, AGA-201, ATI-1777, ATI-501, ESK-001, GLPG-3667, LNK-01001, LNK-01003, R-348, TD-8236, TLL-018, TQ-05105, VTX-958, or a combination thereof.

In some embodiments, the cancer is a B-ALL, and the additional therapy or therapeutic agent is a JAK inhibitor. In some embodiments, the cancer is a JAK2 (e.g., JAK2^{R683G} or JAK2^{I682F}) mutant B-ALL, and the additional therapy or therapeutic agent is a JAK inhibitor. In some embodiments, the cancer is a JAK2 (e.g., JAK2^{R683G} or JAK2^{I682F}) mutant B-ALL with high CRLF2 expression, and the additional therapy or therapeutic agent is a JAK inhibitor and a BCL-2 inhibitor (e.g., venetoclax).

In some embodiments, the BRaf inhibitor is avutometinib (RO5126766), dabrafenib (e.g., dabrafenib mesylate, GSK2118436), encorafenib (e.g., BRAFTOVI™, LGX⁸¹⁸), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, PLX-4720, or a combination thereof.

In some embodiments, the MEK inhibitor is avutometinib (RO5126766), binimetinib (MEKTOVI®, MEK162), cobimetinib (e.g., cobimetinib fumarate, COTELLIC®), mirdametinib (PD0325901), pimasertib (MSC1936369B), refametinib, selumetinib (e.g., selumetinib sulfate, AZD6244), trametinib (e.g., trametinib dimethyl sulfoxide, GSK-1120212), zapnometinib, CI1040 (PD184352), CS3006, FCN-159, GSK-1120212, NFX-179, PD98059, SHR7390, TAK-733, WX-554, or a combination thereof. In some embodiments, the MEK inhibitor is avutometinib, binimetinib, cobimetinib (e.g., cobimetinib fumarate), mirdametinib, pimasertib, refametinib, selumetinib (e.g., selumetinib sulfate), trametinib (e.g., trametinib dimethyl sulfoxide, GSK-1120212), zapnometinib, FCN-159, NFX-179, TAK-733, or a combination thereof.

In some embodiments, the menin inhibitor is revumenib (e.g., revumenib fumarate), ziftomenib, BMF-219, DS-1594, JNJ-6617, or a combination thereof.

In some embodiments, the cancer is a B-ALL, and the additional therapy or therapeutic agent is a menin inhibitor. In some embodiments, the cancer is an MLL-rearranged (e.g., an MLL-Af4 fusion, an MLL-Af6 fusion, an MLL-Af9 fusion, an MLL-ENL fusion, or an MLL-PTD fusion) B-ALL, and the additional therapy or therapeutic agent is a menin inhibitor.

In some embodiments, the BCL-2 inhibitor is lisaftoclax, navitoclax, obatoclax, venetoclax, oblimersen (e.g., oblimersen sodium), beclanorsen, AZD-0466, BGB-11417, UBX-1325, UBX-1967, ZN-d5, or a combination thereof. In some embodiments, the BCL-2 inhibitor is lisaftoclax, navitoclax, obatoclax, venetoclax, oblimersen (e.g., oblimersen sodium), beclanorsen, or a combination thereof.

In some embodiments, the cancer is a B-ALL, and the additional therapy or therapeutic agent is a BCL-2 inhibitor. In some embodiments, the cancer is an MLL-rearranged (e.g., an MLL-Af4 fusion, an MLL-Af6 fusion, an MLL-Af9 fusion, an MLL-ENL fusion, or an MLL-PTD fusion) B-ALL, and the additional therapy or therapeutic agent is a BCL-2 inhibitor. In some embodiments, the cancer is a BCL2 amplified B-ALL, and the additional therapy or therapeutic agent is a BCL-2 inhibitor. In some embodiments, the BCL-2 inhibitor is venetoclax.

In some embodiments, the cancer is a DLBCL, and the additional therapy or therapeutic agent is a BCL-2 inhibitor. In some embodiments, the BCL-2 inhibitor is venetoclax.

In some embodiments, the cancer is a FL, and the additional therapy or therapeutic agent is a BCL-2 inhibitor. In some embodiments, the BCL-2 inhibitor is venetoclax.

In some embodiments, the cancer is an MCL, and the additional therapy or therapeutic agent is a BCL-2 inhibitor. In some embodiments, the BCL-2 inhibitor is venetoclax.

In some embodiments, the BCL-XL inhibitor is lisaftoclax, navitoclax, obatoclax, pelcitoclax, mirzotamab clezutoclax, ABBV-155, APG-1252-12A, AZD-0466, DT-2216, PA-15227, UBX-1325, UBX-1967, XZ-739, 753-B, or a combination thereof. In some embodiments, the BCL-XL inhibitor is lisaftoclax, navitoclax, obatoclax, or a combination thereof.

In some embodiments, the MCL-1 inhibitor is omacetaxine (e.g., omacetaxine mepesuccinate).

In some embodiments, the XPO1 inhibitor is eltanexor, felezonexor, selinexor, verdinexor, BIIB-100, JS-110, or a combination thereof. In some embodiments, the XPO1 inhibitor is selinexor.

In some embodiments, the inhibitor of the PRC2 is lirametostat, tazemetostat (e.g., tazemetostat hydrobromide), tulmimetostat (CPI-0209), valemetostat (e.g., valemetostat tosylate), EBI-2511, HH-2853, HM-97662, PF-6821497, SHR-2554, XNW-5004, or a combination thereof. In some embodiments, the inhibitor of the PRC2 is an EZH1/2 inhibitor, an EZH2 inhibitor, or a combination thereof. Non-limiting examples of EZH2 and/or EZH1/2 inhibitors are described in International Publication Nos. WO 2011/140325, WO 2012/005805, WO 2012/050532, WO 2012/118812, WO 2012/142513, WO 2012/142504, WO 2013/049770, WO 2013/039988, WO 2013/067300, WO 2015/141616, WO 2017/084494, WO 2018/210296, WO 2018/210302, WO 2019/091450, WO 2019/204490, WO 2019/226491, WO 2020/063863, WO 2020/171606, WO 2020/228591, WO 2021/016414, WO 2021/063332, WO 2021/063340, WO 2021/180235, and WO 2022/035303.

In some embodiments, the cancer is a DLBCL, and the additional therapy or therapeutic agent is an inhibitor of the PRC2 (e.g., an EZH1/2 or EZH2 inhibitor). In some embodiments, the cancer is a B-ALL (e.g., Philadelphia chromosome positive B-ALL, Philadelphia chromosome negative B-ALL, or B-ALL with an MLL rearrangement (e.g., an MLL-Af4 fusion, an MLL-Af6 fusion, an MLL-Af9 fusion, an MLL-ENL fusion, or an MLL-PTD fusion)), and the additional therapy or therapeutic agent is an inhibitor of the PRC2. In some embodiments, the cancer is a FL, and the additional therapy or therapeutic agent is an inhibitor of the PRC2 (e.g., an EZH1/2 or EZH2 inhibitor). In some embodiments, the cancer is an MCL, and the additional therapy or therapeutic agent is an inhibitor of the PRC2 (e.g., an EZH1/2 or EZH2 inhibitor).

An exemplary wild-type human EZH2 sequence is shown below. This is one of several isoforms of EZH2, and it will be understood that residue numbering may change based on the reference isoform.

SEQ ID NO: 1 (UniParc ID UPI000006D77C):
MGQTGKKSEKGPVCWRKRVKSEYMRLRQLKRFRRADEVKSMFSSNR
QKILERTEILNQEWKQRRIQPVHILTSVSSLRGTRECSVTSDLDF
PTQVIPLKTLNAVASVPIMYSWSPLQQNFMVEDETVLHNIPYMGD
EVLDQDGTFIEELIKNYDGKVHGDRECGFINDEIFVELVNALGQY
NDDDDDDDGDDPEEREEKQKDLEDHRDDKESRPPRKFPSDKIFEA
ISSMFPDKGTAEELKEKYKELTEQQLPGALPPECTPNIDGPNAKS
VQREQSLHSFHTLFCRRCFKYDCFLHRKCNYSFHATPNTYKRKNT
ETALDNKPCGPQCYQHLEGAKEFAAALTAERIKTPPKRPGGRRRG
RLPNNSSRPSTPTINVLESKDTDSDREAGTETGGENNDKEEEEKK
DETSSSSEANSRCQTPIKMKPNIEPPENVEWSGAEASMFRVLIGT
YYDNFCAIARLIGTKTCRQVYEFRVKESSIIAPAPAEDVDTPPRK
KKRKHRLWAAHCRKIQLKKDGSSNHVYNYQPCDHPRQPCDSSCPC
VIAQNFCEKFCQCSSECQNRFPGCRCKAQCNTKQCPCYLAVRECD
PDLCLTCGAADHWDSKNVSCKNCSIQRGSKKHLLLAPSDVAGWGI
FIKDPVQKNEFISEYCGEIISQDEADRRGKVYDKYMCSFLFNLNN
DFVVDATRKGNKIRFANHSVNPNCYAKVMMVNGDHRIGIFAKRAI
QTGEELFFDYRYSQADALKYVGIEREMEIP

In some embodiments, the steroid is dexamethasone, prednisone, or a combination thereof.

In some embodiments, the immunomodulatory imide drug is avadomide, lenalidomide, iberdomide, pomalidomide, thalidomide, CC-99282, or a combination thereof.

In some embodiments, the additional therapy or therapeutic agent is lenalidomide and rituximab or obinutuzumab.

In some embodiments, the anti-CD19 therapy is blinatumomab (e.g., BLINCYTO® (blinatumomab) or a biosimilar thereof), coltuximab ravtansine, inebilizumab (e.g., inebilizumab-cdon, or a biosimilar thereof), loncastuximab tesirine (e.g., loncastuximab tesirine-lpyl, or a biosimilar thereof), obexelimab, tafasitamab (e.g., tafasitamab-cxix, or a biosimilar thereof), dDT-2219, biosimilars thereof, or a combination thereof. In some embodiments, the anti-CD19 therapy is a bispecific antibody or antigen-binding fragment thereof (e.g., BLINCYTO® (blinatumomab) or a biosimilar thereof). In some embodiments, the anti-CD19 therapy is an anti-CD19 and anti-CD3 bispecific antibody or antigen-binding fragment thereof (e.g., BLINCYTOR (blinatumomab) or a biosimilar thereof). In some embodiments, the anti-CD19 therapy is an antibody-drug conjugate (e.g., coltuximab ravtansine, loncastuximab tesirine (e.g., loncastuximab tesirine-lpyl, or a biosimilar thereof)).

In some embodiments, the anti-CD20 therapy is divozilimab, epcoritamab (e.g., epcoritamab-bysp, or a biosimilar thereof), glofitamab (e.g., COLUMVI® (glofitamab), or a biosimilar thereof), ibritumomab tiuxetan (e.g., ZEVALIN® (ibritumomab tiuxetan), or a biosimilar thereof), mosunetuzumab (e.g., mosunetuzumab-axgb, or a biosimilar thereof), obinutuzumab (e.g., GAZYVAR (obinutuzumab), or a biosimilar thereof), ocrelizumab (e.g., OCREVUS® (ocrelizumab), or a biosimilar thereof), odronextamab, ofatumumab (e.g., ARZERRAR (ofatumumab), or a biosimilar thereof), plamotamab, rituximab (e.g., RITUXAN® (rituximab), or a biosimilar thereof (e.g., rituximab-abbs, rituximab-arrx, rituximab-pvvr, ACELLBIAR (rituximab), HALPRYZAR (rituximab), HANLIKON® (rituximab), RIXATHON® (rituximab), REDITUX™ (rituximab), Retuxira (rituximab), BI-695500, GB-241, Mabion-CD20, RTXM-83, SAIT-101), ublituximab (e.g., ublituximab-xiiy, or a biosimilar thereof), veltuzumab, zuberitamab, MIL-62, SCT-400, TQB-2303, biosimilars thereof, or a combination thereof. In some embodiments, the anti-CD20 therapy is a bispecific antibody or antigen-binding fragment thereof (e.g., epcoritamab (e.g., epcoritamab-bysp, or a biosimilar thereof), glofitamab (e.g., COLUMVI® (glofitamab), or a biosimilar thereof), mosunetuzumab (e.g., mosunetuzumab-axgb, or a biosimilar thereof), plamotamab, odronextamab, biosimilars thereof, or a combination thereof. In some embodiments, the anti-CD20 therapy is an anti-CD20 and anti-CD3 bispecific antibody or antigen-binding fragment thereof (e.g., epcoritamab (e.g., epcoritamab-bysp, or a biosimilar thereof), glofitamab (e.g., COLUMVI® (glofitamab), or a biosimilar thereof), mosunetuzumab (e.g., mosunetuzumab-axgb, or a biosimilar thereof), plamotamab, odronextamab, biosimilars thereof, or a combination thereof. In some embodiments, the anti-CD20 therapy is an antibody-drug conjugate (e.g., ibritumomab tiuxetan (e.g., ZEVALIN® (ibritumomab tiuxetan), or a biosimilar thereof).

In some embodiments, the additional therapy or therapeutic agent is rituximab. In some such embodiments, the combination of rituximab and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is used as maintenance therapy. In some such embodiments, the cancer is a FL.

In some embodiments, the additional therapy or therapeutic agent is obinutuzumab. In some such embodiments, the combination of obinutuzumab and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is used as maintenance therapy. In some such embodiments, the cancer is a FL.

In some embodiments, the cancer is a DLBCL, and the additional therapy or therapeutic agent is anti-CD20 therapy. In some embodiments, the anti-CD20 therapy is rituximab, obinutuzumab, or a combination thereof.

In some embodiments, the cancer is a FL, and the additional therapy or therapeutic agent is anti-CD20 therapy. In some embodiments, the anti-CD20 therapy is rituximab, obinutuzumab, or a combination thereof.

In some embodiments, the anti-CD22 therapy is an antibody-drug conjugate (e.g., inotuzumab ozogamicin.

In some embodiments, the cancer is a B-ALL, and the additional therapy or therapeutic agent is anti-CD22 therapy. In some embodiments, the anti-CD22 therapy is an anti-CD22 antibody-drug conjugate (e.g., inotuzumab ozogamicin, or a biosimilar thereof).

In some embodiments, the anti-CD3 therapy is blinatumomab (e.g., BLINCYTO® (blinatumomab), or a biosimilar thereof), catumaxomab, elranatamab, epcoritamab (e.g., epcoritamab-bysp, or a biosimilar thereof), ertumaxomab, glofitamab (e.g., COLUMVI® (glofitamab), or a biosimilar thereof), linvoseltamab, mosunetuzumab (e.g., mosunetuzumab-axgb, or a biosimilar thereof), odronextamab, otelixizumab, plamotamab, talquetamab, tarlatamab, tebentafusp (e.g., tebentafusp-tebn, or a biosimilar thereof), teclistamab (e.g., teclistamab-cqyv, or a biosimilar thereof), teplizumab (e.g., teplizumab-mzwv, or a biosimilar thereof), visilizumab, biosimilars thereof, or a combination thereof. In some embodiments, the anti-CD3 therapy is bispecific antibody or antigen-binding fragment thereof (e.g., blinatumomab (e.g., BLINCYTO® (blinatumomab), or a biosimilar thereof), catumaxomab, elranatamab, epcoritamab (e.g., epcoritamab-bysp, or a biosimilar thereof), ertumaxomab, glofitamab (e.g., COLUMVI® (glofitamab), or a biosimilar thereof), linvoseltamab, mosunetuzumab (e.g., mosunetuzumab-axgb, or a biosimilar thereof), plamotamab, odronextamab, talquetamab, tarlatamab, tebentafusp (e.g., tebentafusp-tebn, or a biosimilar thereof), teclistamab (e.g., teclistamab-cqyv, or a biosimilar thereof), biosimilars thereof, or a combination thereof. In some embodiments, the anti-CD3 therapy is an anti-CD3 and anti-CD19 bispecific antibody or antigen-binding fragment thereof (e.g., BLINCYTO® (blinatumomab) or a biosimilar thereof). In some embodiments, the anti-CD3 therapy is an anti-CD20 and anti-CD3 bispecific antibody or antigen-binding fragment thereof (e.g., epcoritamab (e.g., epcoritamab-bysp, or a biosimilar thereof), glofitamab (e.g., COLUMVI® (glofitamab), or a biosimilar thereof), mosunetuzumab (e.g., mosunetuzumab-axgb, or a biosimilar thereof), plamotamab, odronextamab, biosimilars thereof, or a combination thereof.

In some embodiments, the anti-CD30 therapy is brentuximab, brentuximab vedotin, iratumumab, AFM-13, biosimilars thereof, or a combination thereof. In some embodiments, the anti-CD30 therapy is a bispecific antibody or antigen-binding fragment thereof (e.g., AFM-13). In some embodiments, the anti-CD30 therapy is an antibody-drug conjugate (e.g., brentuximab vedotin, or a biosimilar thereof).

In some embodiments, the anti-CD79B therapy is polatuzumab (e.g., polatuzumab vedotin (e.g., polatuzumab vedotin-piiq, or a biosimilar thereof)), MGD-010, RG-7986, biosimilars thereof, or a combination thereof. In some embodiments, the anti-CD79B therapy is polatuzumab (e.g., polatuzumab vedotin (e.g., polatuzumab vedotin-piiq, or a biosimilar thereof)), MGD-010, biosimilars thereof, or a combination thereof. In some embodiments, the anti-CD79B therapy is a bispecific antibody or antigen-binding fragment thereof (e.g., MGD-010). In some embodiments, the anti-CD79B therapy is an antibody-drug conjugate (e.g., polatuzumab vedotin (e.g., polatuzumab vedotin-piiq, or a biosimilar thereof)).

In some embodiments, the anti-PD1 therapy is balstilimab, budigalimab, cadonilimab, camrelizumab, cemiplimab (e.g., cemiplimab-rwlc, or a biosimilar thereof), cetrelimab, dostarlimab (e.g., dostarlimab-gxly, or a biosimilar thereof), ezabenlimab, geptanolimab, ivonescimab, nivolumab (e.g., OPDIVO® (nivolumab), or a biosimilar thereof), nofazinlimab, pembrolizumab (e.g., KEYTRUDA® (pembrolizumab), or a biosimilar thereof), penpulimab, pidilizumab, pimivalimab, prolgolimab, pucotenlimab, retifanlimab (e.g., retifanlimab-dlwr, or a biosimilar thereof), rilvegostomig, rosnilimab, rulonilimab, sasanlimab, serplulimab, sintilimab (e.g., TYVYT® (sintilimab), or a biosimilar thereof), spartalizumab, tebotelimab, tislelizumab, toripalimab, volrustomig, vudalimab, zimberelimab, QL-1604, HX-009, INCB-086550, RG-6139, BAT-1306, SG-001, AZD7709, biosimilars thereof, or a combination thereof. In some embodiments, the anti-PD1 therapy is a bispecific antibody or antigen-binding fragment thereof (e.g., cadonilimab, ivonescimab, rilvegostomig, tebotelimab, volrustomig, vudalimab, AZD7709, HX-009, RG-6139, biosimilars thereof, or a combination thereof). In some embodiments, the anti-PD1 therapy is an anti-PD1 and anti-CD47 bispecific antibody or antigen-binding fragment thereof (e.g., HX-009, or a biosimilar thereof).

In some embodiments, the anti-PD-L1 therapy is adebrelimab, atezolizumab (e.g., TECENTRIQ® (atezolizumab), or a biosimilar thereof), avelumab (e.g., BAVENCIO® (avelumab), or a biosimilar thereof), bintrafusp alfa, cosibelimab, danburstotug, durvalumab (e.g., IMFINZI® (durvalumab), or a biosimilar thereof), envafolimab (e.g., ENWEIDA® (envafolimab), or a biosimilar thereof), erfonrilimab, pacmilimab, socazolimab, sugemalimab (e.g., CEJEMLY® (sugemalimab), or a biosimilar thereof), A-167, APL-502, AUPM-170, BNT-311, SHR-1701, biosimilars thereof, or a combination thereof. In some embodiments, the anti-PD-L1 therapy is a bispecific antibody or antigen-binding fragment thereof (e.g., erfonrilimab, BNT-311, biosimilars thereof, or a combination thereof).

In some embodiments, the PD-L1 inhibitor is INCB-086550.

In some embodiments, the anti-CD47 therapy is lemzoparlimab, letaplimab, magrolimab, 6MW-3211, AO-176, CPO-107, HX-009, TTI-621, TTI-622, biosimilars thereof, or a combination thereof. In some embodiments, the anti-CD47 therapy is lemzoparlimab, magrolimab, HX-009, TTI-621, TTI-622, biosimilars thereof, or a combination thereof. In some embodiments, the anti-anti-CD47 therapy is a bispecific antibody or antigen-binding fragment thereof (e.g., HX-009). In some embodiments, the anti-CD47 therapy is an anti-CD47 and anti-PD1 bispecific antibody or antigen-binding fragment thereof (e.g., HX-009, or a biosimilar thereof).

In some embodiments, the cell-based therapy is adoptive cell therapy (e.g., CAR T therapy, cytokine-induced killer cells (CIKs), natural killer cells (e.g., CAR-modified NK cells)) or antibody-armed cell therapy. In some embodiments, the cell-based therapy is CAR T therapy. In some embodiments, the cell-based therapy is axicabtagene ciloleucel (e.g., YESCARTA® (axicabtagene ciloleucel), or a biosimilar thereof), brexucabtagene autoleucel (e.g., TECARTUS® (brexucabtagene autoleucel), or a biosimilar thereof), inaticabtagene autoleucel, lisocabtagene maraleucel (e.g., BREYANZI® (lisocabtagene maraleucel), or a biosimilar thereof), rapcabtagene autoleucel, relmacabtagene autoleucel (e.g., CARTEYVAR (relmacabtagene autoleucel), or a biosimilar thereof), tisagenlecleucel (e.g., KYMRIAH® (tisagenlecleucel), or a biosimilar thereof), varnimcabtagene autoleucel (e.g., IMN-003A, or a biosimilar thereof), zamtocabtagene autoleucel, BZ-019, CD19CAR CTL, CTL-119, TAK-007, XLCART-001, biosimilars thereof, or a combination thereof.

In some embodiments, the additional therapy or therapeutic agent is chemotherapy. In some embodiments, the chemotherapy is CVAD, hyperCVAD (cyclophosphamide, vincristine, doxorubicin, and dexamethasone), CHOP, R-CHOP, G-CHOP, EPOCH, R-EPOCH, Pola-R-CHP (polatuzumab vedotin, rituximab, cyclophosphamide, doxorubicin, and prednisone), R-CODOX-M, R-IVAC, DA-EPOCH-R, CVP, R-CVP, G-CVP, CVD (cyclophosphamide, vincristine, dacarbazine, including mini-CVD), bendamustine with rituximab or obinutuzumab, methotrexate-cytarabine, vincristine (with or without steroids (e.g., dexamethasone)), nelarabine, a hypomethylating agent (e.g., azacitidine and/or decitabine), CALGB8811, or pediatric-inspired multi-agent chemotherapy (e.g., GRAALL-2003, COG AALL-0434, CCG-1961, CALGB 10403, or the DFCI regimen).

In some embodiments, the cancer is a B-ALL, and the additional therapy or therapeutic agent is chemotherapy (e.g., CVAD). In some embodiments, the cancer is an MLL-rearranged (e.g., an MLL-Af4 fusion, an MLL-Af6 fusion, an MLL-Af9 fusion, an MLL-ENL fusion, or an MLL-PTD fusion) B-ALL, and the additional therapy or therapeutic agent is chemotherapy (e.g., CVAD). In some embodiments, the cancer is a Philadelphia chromosome positive B-ALL, and the additional therapy or therapeutic agent is chemotherapy (e.g., CVAD). In some embodiments, the cancer is a Philadelphia chromosome-like B-ALL, and the additional therapy or therapeutic agent is chemotherapy (e.g., CVAD). In some embodiments, the cancer is a pre-BCR+B-ALL, and the additional therapy or therapeutic agent is chemotherapy (e.g., CVAD). In some embodiments, the cancer is a B-ALL dependent on Ras signaling, and the additional therapy or therapeutic agent is a chemotherapy (e.g., CVAD).

In some embodiments, the cancer is a FL, and the additional therapy or therapeutic agent is R-CHOP. In some such embodiments, the combination of R-CHOP and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is used as treatment for a primary tumor. In some such embodiments, the combination of R-CHOP and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is used as maintenance therapy.

In some embodiments, the cancer is a DLBCL, and the additional therapy or therapeutic agent is R-CHOP. In some such embodiments, the combination of R-CHOP and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is used as treatment for a primary tumor. In some such embodiments, the combination of R-CHOP and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is used as maintenance therapy.

In some embodiments, the cancer is a DLBCL, and the additional therapy or therapeutic agent is R-EPOCH. In some such embodiments, the combination of R-EPOCH and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is used as treatment for a primary tumor. In some such embodiments, the combination of R-EPOCH and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is used as maintenance therapy.

In some embodiments, the cancer is a DLBCL, and the additional therapy or therapeutic agent is Pola-R-CHP. In some such embodiments, the combination of Pola-R-CHP and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is used as treatment for a primary tumor. In some such embodiments, the combination of Pola-R-CHP and a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is used as maintenance therapy.

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof is used in combination with one or more steps of induction, consolidation, intensification, or maintenance in a chemotherapeutic regimen.

Also provided herein is a method of treating cancer, comprising administering to a subject in need thereof (a) a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, and (b) an additional therapeutic agent, for simultaneous, separate or sequential use for the treatment of cancer, wherein the amounts of the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, and the additional therapeutic agent are together effective in treating the cancer. In some embodiments, the method comprises administering (c) at least one pharmaceutically acceptable carrier.

In some such embodiments, the additional therapeutic agent is an inhibitor of the PRC2 (e.g., an EZH1/2 inhibitor or an EZH2 inhibitor (e.g., any of the EZH1/2 inhibitors or EZH2 inhibitors described herein)), and the cancer is a cancer having an EZH2 alteration. In an aspect of this embodiment, the EZH2 alteration is a mutation is at residue 27, residue 34, residue 59, residue 141, residue 162, residue 172, residue 197, residue 238, residue 239, residue 246, residue 395, residue 401, residue 452, residue 510, residue 516, residue 556, residue 583, residue 618, residue 644, residue 646, residue 682, residue 690, residue 692, residue 716, residue 732, residue 744, residue 745, or a combination thereof, relative to SEQ ID NO. 1. In another aspect of this embodiment, the EZH2 alteration is a translocation. In another aspect of this embodiment, the EZH2 mutation is R27*, R34*, E59*, Q141*, E162*, V172Cfs*11, E197Rfs*12, E238*, E239*, E246*, G395Efs*29, E401Kfs*22, E401*, Y452*, K510Yfs*3, X516_splice, S556*, R583*, X618_splice, S644*, Y646F, Y646N, Y646S, R690G, R690H, A692V, F716Lfs*24, X732_splice, 1744Mfs*25, E745Afs*24, EZH2-AUTS2, EZH2-TMEM176B, GALNT11-EZH2, or a combination thereof. In another aspect of this embodiment, the EZH2 mutation is at residue 646, residue 682, or residue 692 relative to SEQ ID NO. 1. In another aspect of this embodiment, the EZH2 mutation is Y646F, Y646N, A682G, or A692V relative to SEQ ID NO: 1. In another aspect of this embodiment, the cancer is a lymphoma (e.g., FL or DLBCL) and the EZH2 mutation is Y646F, Y646N, A682G, or A692V relative to SEQ ID NO: 1.

These additional therapeutic agents may be administered with one or more doses of the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, or pharmaceutical composition comprising a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, as part of the same or separate dosage forms, via the same or different routes of administration, and/or on the same or different administration schedules according to standard pharmaceutical practice known to one skilled in the art. In some embodiments, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, and the additional therapeutic agent are administered simultaneously as separate dosages. In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, and the additional therapeutic agent are administered as separate dosages simultaneously, separately, or sequentially in any order, in jointly therapeutically effective amounts, e.g., in daily or intermittent dosages. In some embodiments, the compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, and the additional therapeutic agent are administered simultaneously as a combined dosage. When administered simultaneously, the two agents can be administered as a single dosage form (e.g., a fixed dosage form) or as separate dosages (e.g., non-fixed dosage forms).

As used herein, the terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject,” “individual,” or “patient,” are used interchangeably, and refer to any animal, including mammals such as mice, rats, other rodents, rabbits, dogs, cats, swine, cattle, sheep, horses, primates, and humans. In some embodiments, the subject is a human. In some embodiments, the subject has experienced and/or exhibited at least one symptom of the disease, disorder, or condition to be treated and/or prevented.

In some embodiments, the subject is a pediatric subject.

The term “pediatric subject” as used herein refers to a subject under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

The term “preventing” as used herein means to delay the onset, recurrence or spread, in whole or in part, of the disease or condition as described herein, or a symptom thereof.

The term “regulatory agency” refers to a country's agency for the approval of the medical use of pharmaceutical agents with the country. For example, a non-limiting example of a regulatory agency is the U.S. Food and Drug Administration (FDA).

The phrase “therapeutically effective amount” means an amount of compound that, when administered to a subject in need thereof, is sufficient to (i) treat a disease, disorder, or condition, (ii) attenuate, ameliorate, or eliminate one or more symptoms of the particular disease, disorder, or condition, or (iii) delay the onset of one or more symptoms of the particular disease, disorder, or condition described herein. The amount of a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, that will correspond to such an amount will vary depending upon factors such as the particular compound, disease condition and its severity, the identity (e.g., weight) of the subject in need of treatment, but can nevertheless be routinely determined by one skilled in the art.

### Pharmaceutical Compositions and Administration

### General

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, is administered as a pharmaceutical composition that includes the compound, or a pharmaceutically acceptable salt thereof, and one or more pharmaceutically acceptable excipients, and optionally one or more additional therapeutic agents as described herein.

In some embodiments, the compounds can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-a-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a compound as described herein in the range of 0.005% to 100% with the balance made up from non-toxic excipient may be prepared. The contemplated compositions may contain 0.001%-100% of a compound provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, a compound of Formula (I) (e.g., Formula (I-1), Formula (I-a) (e.g., Formula (I-a-1), (I-a-2), (I-a-3), (I-a-4), (I-a-5), or (I-a-6)), or Formula (I-b) (e.g., Formula (I-b-1) or (I-b-2))), or a pharmaceutically acceptable salt thereof, as described herein, or a pharmaceutical composition thereof, can be administered to a subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

In some embodiments, a compound of Formula (I), or a pharmaceutically acceptable salt thereof, as described herein, or a pharmaceutical composition thereof, can be administered orally to a subject in need thereof. Without being bound by any particular theory, it is believed that oral dosing (e.g., versus IV dosing) can be preferred by patients for convenience, perception of efficacy, and/or past experience.

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the compound described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein, or a pharmaceutical composition thereof, are suitable for local delivery to the digestive or GI tract by way of oral administration (e.g., solid or liquid dosage forms.).

Solid dosage forms for oral administration include capsules, tablets, pills, powders, and granules. In such solid dosage forms, the compound is mixed with one or more pharmaceutically acceptable excipients, such as sodium citrate or dicalcium phosphate and/or: a) fillers or extenders such as starches, lactose, sucrose, glucose, mannitol, and silicic acid, b) binders such for example, as, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a compound, or pharmaceutically acceptable salt thereof, as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives, or the like. In another solid dosage form, a powder, marume, solution or suspension (e.g., in propylene carbonate, vegetable oils, PEGs, poloxamer 124 or triglycerides) is encapsulated in a capsule (gelatin or cellulose base capsule). Unit dosage forms in which one or more compounds provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can include one or more components that chemically and/or structurally predispose the composition for delivery of the compounds to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier, and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic, or amphoteric surfactant. As with other carriers or vehicles, an ointment base should be inert, stable, nonirritating, and non-sensitizing.

In any of the foregoing embodiments, pharmaceutical compositions described herein can include one or more one or more of the following: lipids, interbilayer crosslinked multilamellar vesicles, biodegradable poly (D,L-lactic-co-glycolic acid) [PLGA]-based or poly anhydride-based nanoparticles or microparticles, and nanoporous particle-supported lipid bilayers.

### Dosages

The dosages may be varied depending on the requirement of the patient, the severity of the condition being treated, and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 1 mg/kg; from about 0.1 mg/kg to about 0.5 mg/kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

The term “acceptable” with respect to a formulation, composition, or ingredient, as used herein, means having no persistent detrimental effect on the general health of the subject being treated.

“API” refers to an active pharmaceutical ingredient.

The term “excipient” or “pharmaceutically acceptable excipient” means a pharmaceutically acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid, and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The term “pharmacologically acceptable salts” is not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine, and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid: organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to a subject. Multiple techniques of administering a compound exist in the art including, but not limited to: rectal, oral, intravenous, aerosol, parenteral, ophthalmic, pulmonary, and topical administration.

### Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

Also provided herein are methods of preparing compounds of Formula (I-a) as depicted in Scheme 1,
the methods comprising reacting compounds of Formula (SII) with compounds of Formula (SI), to provide compounds of Formula (I-a):

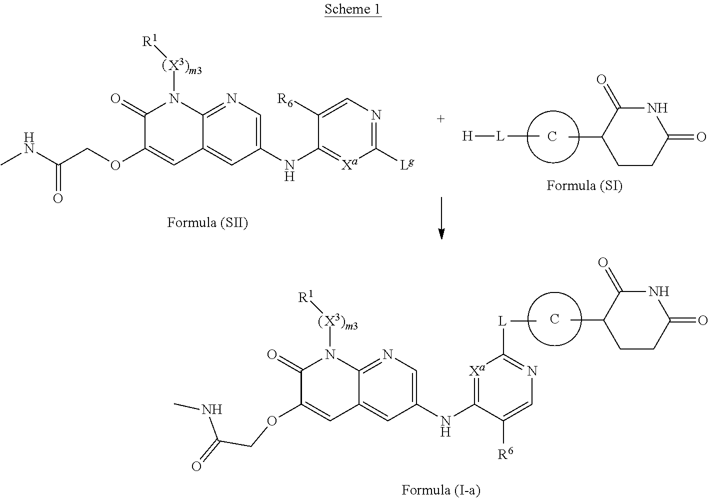

wherein:
Lg is halo;
X^{a} is N or CH;
R⁶ is —F or —Cl;
m3 is 1, X³ is C₁₋₃ alkylene, and R¹ is H;
Ring C is selected from the group consisting of:

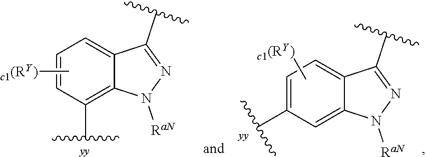

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L is selected from the group consisting of:

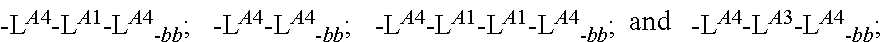

wherein bb represents the point of attachment to Ring C; and
L^{A1} is CH₂, CHMe, or CMe₂;
each L^{A4} is independently 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In the methods of preparing compounds of Formula (I-a) as depicted in Scheme 1, X³, m3, R¹, X^{a}, R⁶, L, and Ring C can be as defined herein for Formula (I-a). In some embodiments of the methods, the compounds of Formula (I-a) are compounds of Formula (I-a-1). In some embodiments of the methods, the compounds of Formula (I-a) are compounds of Formula (I-a-2). In some embodiments of the methods, the compounds of Formula (I-a) are compounds of Formula (I-a-3). In some embodiments of the methods, the compounds of Formula (I-a) are compounds of Formula (I-a-4). In some embodiments of the methods, the compounds of Formula (I-a) are compounds of Formula (I-a-5). In some embodiments of the methods, the compounds of Formula (I-a) are compounds of Formula (I-a-6).

In some embodiments, the methods comprise reacting the compounds of Formula (SII) with the compounds of Formula (SI) under conditions suitable for SNAr reactions. In some embodiments, the methods comprise reacting the compounds of Formula (SII) with the compounds of Formula (SI) in the presence of a base (e.g., triethylamine or diisopropylethylamine) in an appropriate solvent. In some embodiments, the solvent is a polar aprotic solvent (e.g., DMSO or MeCN). In some embodiments, the methods comprise heating the compounds of Formula (SII) with the compounds of Formula (SI) at a temperature of at least 60° C. (e.g., heating the reaction at 60-135° C., 70-120° C., 70-110° C., or 80-110° C.).

Also provided are compounds of Formula (SI):

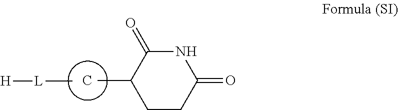

or salts thereof, wherein:

Ring C is selected from the group consisting of:

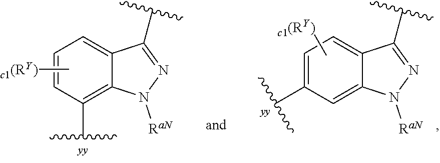

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L is selected from the group consisting of:

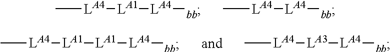

wherein bb represents the point of attachment to Ring C; and
L^{A1} is CH₂, CHMe, or CMe₂;
each L^{A4} is independently 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

Also provided are compounds of Formula (SII):

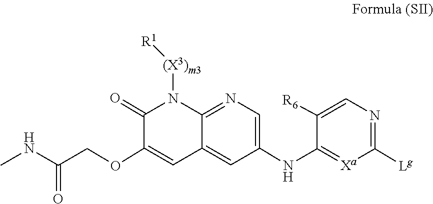

or salts thereof, wherein:
Lg is halo;
X^{a} is N or CH;
R⁶ is —F or —Cl;
m3 is 1, X³ is C₁₋₃ alkylene, and
R¹ is H.

In some embodiments of Formula (SII), X^{a} is N.

In some embodiments of Formula (SII), X^{a} is CH.

In some embodiments of Formula (SII), Le is —Cl.

In some embodiments of Formula (SII), R⁶ is —Cl. In some embodiments of Formula (SII), R⁶ is —F.

### EXAMPLES

In some of the examples disclosed herein, the final product of a described chemical reaction sequence is structurally depicted with enhanced stereochemical notation(s) at one or more stereogenic center(s). Examples of such notations include or1, or2, and the like. In some such examples, in the chemical name of the same compound, each of such stereogenic center(s) is assigned a tentative configuration (e.g., (R)- or (S)-) based on the wedge/dash representation of the structural formula. However, the stereogenic center(s) should be understood to have configurations consistent with the enhanced stereochemical notation(s), as described herein. Accordingly, unless otherwise specified, starting materials and intermediates leading to these compounds incorporate the enhanced stereochemical notations at the corresponding stereogenic centers, notwithstanding the tentative assignments provided in their chemical names.

For example, based on the or1 and or2 notations found on the vicinal stereogenic centers, Compound 156a in Example 48 is a single stereoisomer selected from:
2-((6-((5-chloro-2-((6S,7S)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide;
2-((6-((5-chloro-2-((6S,7R)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide;
2-((6-((5-chloro-2-((6R,7S)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide; and
2-((6-((5-chloro-2-((6R,7R)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide.

The intermediate product provided in the first step of the same example incorporates the orl and or 2 notations. It is therefore a single stereoisomer selected from:
tert-butyl-(6S,7S)-7-((3-(2,6-dioxopiperidin-3-yl)-1-methyl-1H-indazol-6-yl)amino)-6-methyl-2-azaspiro[3.5]nonane-2-carboxylate;
tert-butyl-(6S,7R)-7-((3-(2,6-dioxopiperidin-3-yl)-1-methyl-1H-indazol-6-yl)amino)-6-methyl-2-azaspiro[3.5]nonane-2-carboxylate;
tert-butyl-(6R,7S)-7-((3-(2,6-dioxopiperidin-3-yl)-1-methyl-1H-indazol-6-yl)amino)-6-methyl-2-azaspiro[3.5]nonane-2-carboxylate; and
tert-butyl-(6R,7R)-7-((3-(2,6-dioxopiperidin-3-yl)-1-methyl-1H-indazol-6-yl)amino)-6-methyl-2-azaspiro[3.5]nonane-2-carboxylate.

For further clarification, a chemical name that takes into account the enhanced stereochemical notation(s) (e.g., or1, or2) is provided for the final product having an enhanced stereochemical notation(s) at one or more stereogenic centers. This chemical name is enclosed in brackets (i.e., “[ ]”). In these chemical names, the prefix “rel” means that the stereochemical configuration shown in a chemical name is relative. To illustrate, when a compound contains one stereogenic center, and its chemical name starts with the prefix “rel,” then this stereogenic center is resolved, but its absolute configuration is either (R)- or (S)-. As such, it should be labelled with an or1 enhanced stereochemical notation in its corresponding structure. For example, the chemical name that takes into account the enhanced stereochemical notation for Compound 194a is:
[rel-2-((6-((5-chloro-2-(4-((1S)-1-(1-(3-(2,6-dioxopiperidin-3-yl)-1-methyl-1H-indazol-7-yl)piperidin-4-yl)-1-hydroxyethyl)piperidin-1-yl)pyrimidin-4-yl)amino)-1-isopropyl-2-oxo-1,2-dihydro-1,8-naphthyridin-3-yl)oxy)-N-methylacetamide], as described in Example 76.

When a compound contains two stereogenic centers, and its chemical name starts with the prefix “rel,” the two stereocenters have both been resolved, but they may have (R) or(S) configurations, either concertedly or independently, in conformity with the orx (e.g., or1 or or2) notations in Table C1 for the corresponding compound.

When two stereogenic centers are labelled or1 and or1 in a compound structure, and its chemical name is prefixed with “rel,” then the relative stereochemistry between the two stereogenic centers (e.g., syn or anti relationship) is as represented by the name, but the absolute configurations of the two stereogenic centers can vary concertedly (e.g., a compound designated rel-(R,R) is one stereoisomer selected from (R,R) and (S,S); for avoidance of doubt, the compound designated rel-(R,R) does not have (R,S) or (S,R) configurations across these two stereogenic centers). For example, the chemical name that takes into account the enhanced stereochemical notations for Compound 185a is:
[rel-2-((6-((5-chloro-2-((6R,7S)-7-((3-(2,6-dioxopiperidin-3-yl)-1-methyl-1H-indazol-6-yl)oxy)-6-methyl-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide], as described in Example 72.

When two stereocenters are labelled orl and or2 in a compound structure, and its chemical name is prefixed with “rel,” then the configuration of the two stereocenters can vary independently (e.g., a compound designated rel-(R,R) is one stereoisomer selected from (R,R), (S,S), (R,S), and (S,R)). For example, a chemical name that takes into account the enhanced stereochemical notations for Compound 156a is:
[rel-2-((6-((5-chloro-2-((6S,7S)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide], as described in Example 48.

### Example 1. Synthesis of 2-((6-((5-chloro-2-(4-((1-(3-(2,6-dioxopiperidin-3-yl)-1-methyl-1H-indazol-7-yl)piperidin-4-yl)methyl) piperazin-1-yl)pyrimidin-4-yl)amino)-1-methyl-2-oxo-1,2-dihydro-1,8-naphthyridin-3-yl)oxy)-N-methylacetamide (Compound 101)

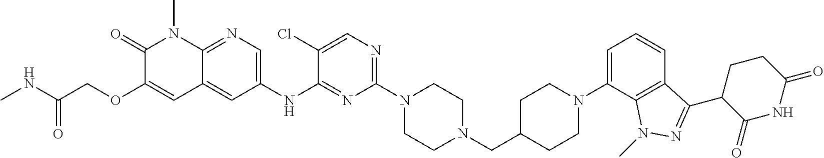 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)cnc2n(C)c1=O]

### Preparation of 1-methyl-5-nitro-1H-pyrrolo[2,3-b]pyridine

To a solution of 5-nitro-1H-pyrrolo[2,3-b]pyridine (50.0 g, 0.30 mol) in DMF (1000 mL) was added potassium carbonate (63.5 g, 0.46 mol). Methyl iodide (65.3 g, 0.46 mol) was added dropwise over a period of 20 minutes and the reaction was stirred at room temperature for 3 hours. The reaction mixture was quenched with ice-cold water (1000 mL), and the precipitated solid was filtered and dried under vacuum to afford 1-methyl-5-nitro-1H-pyrrolo[2,3-b]pyridine (40 g) as a pale-yellow solid.

LC-MS (ESI): m/z=177.95 [M+H]⁺.

### Preparation of 1-methyl-5-nitro-1H-pyrrolo[2,3-b]pyridine-2,3-dione

To a stirred solution of 1-methyl-5-nitro-1H-pyrrolo[2,3-b]pyridine (40.0 g, 0.23 mol) in acetone (280 mL) was added slowly a solution of CrO₃ (293.5 g, 2.93 mol) in a mixture of acetic acid (1280 mL) and H₂O (400 mL) at 0° C. After stirring at room temperature for 3 hours, the reaction mixture was diluted with water (500 mL) and extracted with DCM (500 mL×4). The separated organic layers were combined, washed with brine solution (200 mL), dried over Na₂SO₄, filtered, and concentrated to afford 1-methyl-5-nitro-1H-pyrrolo[2,3-b]pyridine-2,3-dione (28 g) as a pale yellow solid.

LC-MS (ESI): m/z=226.23 [M+18]⁺.

### Preparation of 3-hydroxy-1-methyl-6-nitro-1,8-naphthyridin-2 (1H)-one

To a cooled solution of 1-methyl-5-nitro-1H-pyrrolo[2,3-b]pyridine-2,3-dione (28.0 g, 0.14 mol) in EtOH (560 mL) was added triethyl amine (39 mL, 0.28 mol) and (trimethylsilyl diazomethane (2.0 M in diethyl ether) (32.4 g, 0.28 mol) at 0° C. The reaction was allowed to warm to room temperature over 4 hours while stirring. The reaction mixture was quenched with water (300 mL) and extracted with ethyl acetate (300 mL×3). The combined organic layers were washed with brine solution (100 mL), dried over Na₂SO₄, filtered, and concentrated under reduced pressure to afford 3-hydroxy-1-methyl-6-nitro-1,8-naphthyridin-2 (1H)-one (22 g) as a brown solid.

LC-MS (ESI): m/z=222.28 [M+H]⁺.

### Preparation of N-methyl-2-((1-methyl-6-nitro-2-oxo-1,2-dihydro-1,8-naphthyridin-3-yl)oxy) acetamide

To a stirred solution of 3-hydroxy-1-methyl-6-nitro-1,8-naphthyridin-2 (1H)-one (22 g, 0.10 mol) in DMF (440 mL) was added cesium carbonate (97.7 g, 0.3 mol) and 2-bromo-N-methylacetamide (18.2 g, 0.12 mol). After stirring at room temperature for 3 hours, the reaction was quenched with ice-cold water (500 mL) and extracted with 10% MeOH in DCM (300 mL×3). The separated organic layers were washed with brine solution, dried over Na₂SO₄, filtered, and concentrated to afford N-methyl-2-((1-methyl-6-nitro-2-oxo-1,2-dihydro-1,8-naphthyridin-3-yl)oxy) acetamide (3.0 g) as an off brown solid.

LC-MS (ESI): m/z=293.4 [M+H]⁺.

### Preparation of 2-((6-amino-1-methyl-2-oxo-1,2-dihydro-1,8-naphthyridin-3-yl)oxy)-N-methylacetamide

To a solution of N-methyl-2-((1-methyl-6-nitro-2-oxo-1,2-dihydro-1,8-naphthyridin-3-yl)oxy) acetamide (3.0 g, 0.01 mol) in EtOH (90 mL) was added Zn (6.70 g, 0.1 mol) and acetic acid (5.9 mL, 0.10 mol). After stirring at room temperature for 3 hours, the reaction mixture was diluted with DCM (100 mL) and filtered through a pad of celite. The collected filtrate was concentrated under reduced pressure to afford 2-((6-amino-1-methyl-2-oxo-1,2-dihydro-1,8-naphthyridin-3-yl)oxy)-N-methylacetamide (2.8 g) as a pale yellow solid.

LC-MS (ESI): m/z=263.22 [M+H]⁺.

### Preparation of 2-((6-((2,5-dichloropyrimidin-4-yl)amino)-1-methyl-2-oxo-1,2-dihydro-1,8-naphthyridin-3-yl)oxy)-N-methylacetamide

To a stirred solution of 2-((6-amino-1-methyl-2-oxo-1,2-dihydro-1,8-naphthyridin-3-yl)oxy)-N-methylacetamide (1.0 g, 3.81 mmol) in DMSO (20 mL) was added N,N-diisopropylethylamine (5.5 mL, 30.48 mol) and 2,4,5-trichloropyrimidine (0.84 g, 4.58 mmol). The reaction was heated at 100° C. for 4 hours. The reaction was quenched with ice water and stirred for 20 minutes. The resultant solids were filtered and dried to afford 2-((6-((2,5-dichloropyrimidin-4-yl)amino)-1-methyl-2-oxo-1,2-dihydro-1,8-naphthyridin-3-yl)oxy)-N-methylacetamide (0.1 g) as a light yellow solid.

LC-MS (ESI): m/z=409.04 [M+H]⁺.

### Preparation of tert-butyl 4-((1-((benzyloxy) carbonyl)piperidin-4-yl)methyl) piperazine-1-carboxylate

To a stirred solution of tert-butyl piperazine-1-carboxylate (25.0 g, 0.13 mmol), benzyl 4-formylpiperidine-1-carboxylate (39.60 g, 0.16 mmol), acetic acid (7.80 g, 0.13 mmol) in DCM (500 mL) was added NaBH (OAc)₃ (42.5 g, 0.20 mmol) at 25° C., and the resulting mixture was stirred at 25° C. for 3 hours. The reaction mixture was quenched with water and extracted with DCM. The organic layer was separated, dried over anhydrous sodium sulfate, filtered and concentrated. Crude was purified by column chromatography (SiO₂, 100-200 mesh, 15% EtOAc in petroleum ether) to afford tert-butyl 4-((1-((benzyloxy) carbonyl)piperidin-4-yl)methyl) piperazine-1-carboxylate (25 g) as a pale-yellow solid.

LC-MS (ESI): m/z=418.12 [M+H];

### Preparation of tert-butyl 4-(piperidin-4-ylmethyl) piperazine-1-carboxylate

To a stirred solution of tert-butyl 4-((1-((benzyloxy) carbonyl)piperidin-4-yl)methyl) piperazine-1-carboxylate (25 g, 0.06 mmol) in MeOH (500 mL) was added Pd/C (7.5 g), and the reaction mixture was stirred under hydrogen atmosphere. The resulting mixture was stirred at room temperature for 16 hours. The reaction mixture was diluted with 30% methanol in DCM, filtered through a pad of celite, and the filtrate was collected and concentrated to afford tert-butyl 4-(piperidin-4-ylmethyl) piperazine-1-carboxylate. Crude was purified by trituration using hexane and diethyl ether to afford tert-butyl 4-(piperidin-4-ylmethyl) piperazine-1-carboxylate (7.0 g) as a yellow solid.

¹H NMR (400 MHz, DMSO-d₆): δ 3.29 (s, 4H), 3.11-3.08 (m, 2H), 2.66 (t, J=11.60 Hz, 2H), 2.27-2.25 (m, 4H), 2.11 (d, J=6.80 Hz, 2H), 1.74-1.71 (m, 3H), 1.39 (s, 9H), 1.17-1.14 (m, 2H).

### Preparation of 7-bromo-3-iodo-1-methyl-indazole

To a solution of 7-bromo-3-iodo-1H-indazole (20 g, 61.93 mmol, 1.0 eq.) in THF (50 mL) was added t-BuOK (13.90 g, 123.87 mmol, 2.0 eq.) at 0° C. for 1 hour. CH₃I (26.37 g, 185.80 mmol, 11.57 mL, 3.0 eq.) was added to the mixture at 0° C. The mixture was stirred at 20° C. for 2 hours under N₂ atmosphere. The reaction mixture was filtered and concentrated under reduced pressure to obtain a residue. The crude product was purified by reverse-phase HPLC to afford 7-bromo-3-iodo-1-methyl-indazole (15 g) as a yellow solid.

LC-MS (ESI): m/z=336.6 [M+H]⁺.

### Preparation of 7-bromo-3-(2,6-dibenzyloxy-3-pyridyl)-1-methyl-indazole

A mixture of 2,6-dibenzyloxy-3-(4,4,5,5-tetramethyl-1,3,2-dioxaborolan-2-yl)pyridine (17.34 g, 41.55 mmol, 1.0 eq.), 7-bromo-3-iodo-1-methyl-indazole (14 g, 41.55 mmol, 1.0 eq.), cyclopentyl (diphenyl) phosphane; dichloromethane; dichloropalladium; iron (6.79 g, 8.31 mmol, 0.2 eq.), Cs2CO₃ (40.61 g, 124.65 mmol, 3.0 eq.) in THF (200 mL) and H₂O (40 mL) was degassed and purged with N2 3 times. The mixture was stirred at 85° C. for 1 hour under N₂ atmosphere. The reaction mixture was concentrated under reduced pressure to remove solvent. The resulting residue was purified by column chromatography (SiO₂, petroleum ether/ethyl acetate=1/0 to 10/1) to afford 7-bromo-3-(2,6-dibenzyloxy-3-pyridyl)-1-methyl-indazole (15.1 g) as a white solid.

LC-MS (ESI): m/z=502.3 [M+H]⁺.

### Preparation of tert-butyl 4-((1-(3-(2,6-bis(benzyloxy)pyridin-3-yl)-1-methyl-1H-indazol-7-yl)piperidin-4-yl)methyl) piperazine-1-carboxylate

To a stirred solution of tert-butyl 4-(piperidin-4-ylmethyl) piperazine-1-carboxylate (0.34 g, 1.20 mmol) in 1,4-dioxane (10 mL) was added 7-bromo-3-(2,6-dibenzyloxy-3-pyridyl)-1-methyl-indazole (0.50 g, 1.00 mmol), purged with argon for 15 minutes; cesium carbonate (0. 98 g, 3.00 mmol) and Pd-PEPPSI-iHeptCl (0.05 g, 0.05 mmol) were added, and the mixture was heated to 120° C. for 3 hours. The reaction mixture quenched with water and extracted into ethyl acetate, and the combined organic layers were washed with brine and dried over anhydrous Na₂SO₄, filtered and concentrated under vacuum to get crude. Crude purified by flash column chromatography (SiO₂, 100-200 mesh, 27% ethyl acetate in petroleum ether) to afford tert-butyl 4-((1-(3-(2,6-bis(benzyloxy)pyridin-3-yl)-1-methyl-1H-indazol-7-yl)piperidin-4-yl)methyl) piperazine-1-carboxylate (0.48 g) as a pale yellow solid. LC-MS (ESI): m/z=703.5 [M+H]⁺.

### Preparation of 3-(2,6-bis(benzyloxy)pyridin-3-yl)-1-methyl-7-(4-(piperazin-1-ylmethyl)piperidin-1-yl)-1H-indazole hydrochloride

To a stirred solution of tert-butyl 4-((1-(3-(2,6-bis(benzyloxy)pyridin-3-yl)-1-methyl-1H-indazol-7-yl)piperidin-4-yl)methyl) piperazine-1-carboxylate (0.48 g, 0.68 mmol) in DCM (4.8 mL) was added 4 M HCl in dioxane (2.8 mL) at 0° C., and the reaction mixture was stirred at room temperature for 16 hours. The reaction mixture concentrated under vacuum to afford 3-(2,6-bis(benzyloxy)pyridin-3-yl)-1-methyl-7-(4-(piperazin-1-ylmethyl)piperidin-1-yl)-1H-indazole hydrochloride (0.45 g) as an off-white semi-solid.

¹H NMR (400 MHz, DMSO-d₆): δ 7.84 (d, J=8.00 Hz, 1H), 7.48 (d, J=1.60 Hz, 2H), 7.41-7.42 (m, 9H), 7.30-7.31 (m, 1H), 6.57-6.59 (m, 1H), 5.43 (d, J=5.60 Hz, 4H), 4.32 (s, 3H), 3.27-3.25 (m, 2H), 3.08 (s, 4H), 2.54-2.57 (m, 1H), 2.51-2.51 (m, 3H), 2.28-2.30 (m, 1H).

### Preparation of 3-(1-methyl-7-(4-(piperazin-1-ylmethyl)piperidin-1-yl)-1H-indazol-3-yl)piperidine-2,6-dione

To a stirred solution of 3-(2,6-bis(benzyloxy)pyridin-3-yl)-1-methyl-7-(4-(piperazin-1-ylmethyl)piperidin-1-yl)-1H-indazole hydrochloride (0.45 g, 0.75 mmol) in DMF (4.50 mL) was added 10% palladium on carbon (dry) (0.45 g) and stirred under hydrogen purging with bladder pressure at room temperature for 16 hours. The reaction mixture was diluted with DCM and filtered through a pad of celite and washed with an excess of 30% THF in DCM. Filtrate was collected and concentrated under vacuum to afford crude 3-(1-methyl-7-(4-(piperazin-1-ylmethyl)piperidin-1-yl)-1H-indazol-3-yl)piperidine-2,6-dione (0.45 g, crude) as a pale yellow solid. The crude product was triturated with methanol and diethyl ether to afford 3-(1-methyl-7-(4-(piperazin-1-ylmethyl)piperidin-1-yl)-1H-indazol-3-yl)piperidine-2,6-dione (0.45 g) as a pale yellow solid. LC-MS (ESI): m/z=425 [M+H]⁺.

### Preparation of 2-((6-((5-chloro-2-(4-((1-(3-(2,6-dioxopiperidin-3-yl)-1-methyl-1H-indazol-7-yl)piperidin-4-yl)methyl) piperazin-1-yl)pyrimidin-4-yl)amino)-1-methyl-2-oxo-1,2-dihydro-1,8-naphthyridin-3-yl)oxy)-N-methylacetamide

To a stirred solution of 3-(1-methyl-7-(4-(piperazin-1-ylmethyl)piperidin-1-yl)-1H-indazol-3-yl)piperidine-2,6-dione (0.19 g, 0.45 mmol) in DMSO (3.8 mL) was added 2-((6-((2,5-dichloropyrimidin-4-yl)amino)-1-methyl-2-oxo-1,2-dihydro-1,8-naphthyridin-3-yl)oxy)-N-methylacetamide (0.09 g, 0.22 mmol) and DIPEA (1.0 mL, 5.37 mmol). The reaction was heated at 100° C. for 18 hours. The reaction mixture was concentrated using a centrifugal evaporator to obtain the crude product. Crude product was purified by preparative HPLC to afford 2-((6-((5-chloro-2-(4-((1-(3-(2,6-dioxopiperidin-3-yl)-1-methyl-1H-indazol-7-yl)piperidin-4-yl)methyl) piperazin-1-yl)pyrimidin-4-yl)amino)-1-methyl-2-oxo-1,2-dihydro-1,8-naphthyridin-3-yl)oxy)-N-methylacetamide (0.026 g) as an off-white solid.

LC-MS (ESI): m/z=795.55 [M−H]⁻.

### Example 2. Synthesis of 2-((6-((5-chloro-2-(4-((1-(2-(2,6-dioxopiperidin-3-yl)-1,3-dioxoisoindolin-4-yl)piperidin-4-yl)methyl) piperazin-1-yl)pyrimidin-4-yl)amino)-1-methyl-2-oxo-1,2-dihydro-1,8-naphthyridin-3-yl)oxy)-N-methylacetamide (Compound 102)

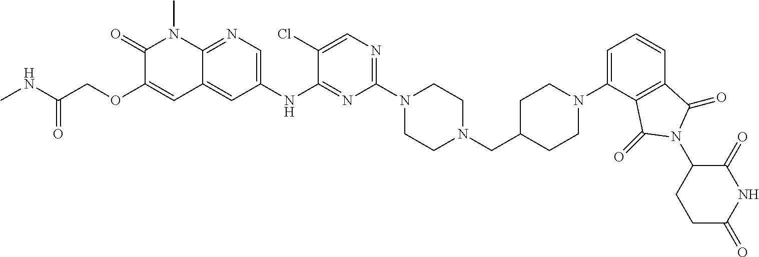 [SMILES: CNC(=O)COc1cc2cc(Nc3nc(N4CCN(CC5CCN(c6cccc7c6C(=O)N(C6CCC(=O)NC6=O)C7=O)CC5)CC4)ncc3Cl)cnc2n(C)c1=O]

### Preparation of tert-butyl 4-((1-(2-(2,6-dioxopiperidin-3-yl)-1,3-dioxoisoindolin-4-yl)piperidin-4-yl)methyl) piperazine-1-carboxylate

To a stirred solution of tert-butyl 4-(piperidin-4-ylmethyl) piperazine-1-carboxylate (1.15 g, 4.06 mmol) in DMSO (5.0 mL) was added 2-(2,6-dioxopiperidin-3-yl)-4-fluoroisoindoline-1,3-dione (0.57 g, 2.07 mmol) and DIPEA (3.45 mL, 20.29 mmol). The reaction was heated to 80° C. for 4 h. The reaction mixture was quenched with ice-cold water, and the precipitated solid was filtered and dried to obtain crude product. The crude product was washed with n-pentane (30 mL) to afford tert-butyl 4-((1-(2-(2,6-dioxopiperidin-3-yl)-1,3-dioxoisoindolin-4-yl)piperidin-4-yl)methyl) piperazine-1-carboxylate (0.90 g) as a yellow semi-solid. LC-MS (ESI): m/z=540.58 [M+H]⁺.

### Preparation of 2-(2,6-dioxopiperidin-3-yl)-4-(4-(piperazin-1-ylmethyl)piperidin-1-yl) isoindoline-1,3-dione

To a stirred solution of tert-butyl 4-((1-(2-(2,6-dioxopiperidin-3-yl)-1,3-dioxoisoindolin-4-yl)piperidin-4-yl)methyl) piperazine-1-carboxylate (0.55 g, 1.02 mmol) in DCM (6.0 mL) was added TFA (2.5 mL), and the reaction was stirred at room temperature for 1 hour. The reaction mixture was concentrated under vacuum to obtain crude product. The crude product was washed with n-pentane (10 mL) and dried under vacuum to afford 2-(2,6-dioxopiperidin-3-yl)-4-(4-(piperazin-1-ylmethyl)piperidin-1-yl) isoindoline-1,3-dione (0.66 g) as a pale green semi-solid. LC-MS (ESI): m/z=440.59 [M+H]⁺

### Preparation of 2-((6-((5-chloro-2-(4-((1-(2-(2,6-dioxopiperidin-3-yl)-1,3-dioxoisoindolin-4-yl)piperidin-4-yl)methyl) piperazin-1-yl)pyrimidin-4-yl)amino)-1-methyl-2-oxo-1,2-dihydro-1,8-naphthyridin-3-yl)oxy)-N-methylacetamide

To a stirred solution of 2-(2,6-dioxopiperidin-3-yl)-4-(4-(piperazin-1-ylmethyl)piperidin-1-yl) isoindoline-1,3-dione (0.20 g, 0.45 mmol) in DMSO (4.0 mL) was added 2-((6-((2,5-dichloropyrimidin-4-yl)amino)-1-methyl-2-oxo-1,2-dihydro-1,8-naphthyridin-3-yl)oxy)-N-methylacetamide (0.09 g, 0.23 mmol) and DIPEA (2.0 mL). The reaction was heated to 110° C. for 8 hours. The reaction mixture was quenched with ice-cold water, and the precipitated solid was filtered and dried to obtain crude product. The crude product was purified by prep-HPLC to afford 2-((6-((5-chloro-2-(4-((1-(2-(2,6-dioxopiperidin-3-yl)-1,3-dioxoisoindolin-4-yl)piperidin-4-yl)methyl) piperazin-1-yl)pyrimidin-4-yl)amino)-1-methyl-2-oxo-1,2-dihydro-1,8-naphthyridin-3-yl)oxy)-N-methylacetamide (0.03 g) as pale yellow solid.

LC-MS (ESI): m/z=812.49 [M+H]⁺.

¹H NMR: (400 MHz, DMSO-d₆): δ 11.07 (s, 1H), 9.09 (s, 1H), 8.70 (d, J=2.40 Hz, 1H), 8.31 (d, J=2.40 Hz, 1H), 8.09 (s, 1H), 7.95-7.94 (m, 1H), 7.69-7.65 (m, 1H), 7.34-7.31 (m, 2H), 7.18 (s, 1H), 5.10-5.06 (m, 1H), 4.62 (s, 2H), 3.73 (s, 3H), 3.69 (d, J=11.20 Hz, 2H), 3.61 (s, 4H), 2.91-2.84 (m, 3H), 2.66 (d, J=4.80 Hz, 3H), 2.61-2.56 (m, 2H), 2.39 (s, 4H), 2.21 (d, J=6.40 Hz, 2H), 2.07-2.02 (m, 1H), 1.90-1.81 (m, 3H), 1.34-1.32 (m, 2H).

### Example 3. Synthesis of 2-((6-((5-chloro-2-(4-((1-(3-(2,6-dioxopiperidin-3-yl)-1-methyl-1H-indazol-6-yl)piperidin-4-yl)methyl) piperazin-1-yl)pyrimidin-4-yl)amino)-1-methyl-2-oxo-1,2-dihydro-1,8-naphthyridin-3-yl)oxy)-N-methylacetamide (Compound 107)

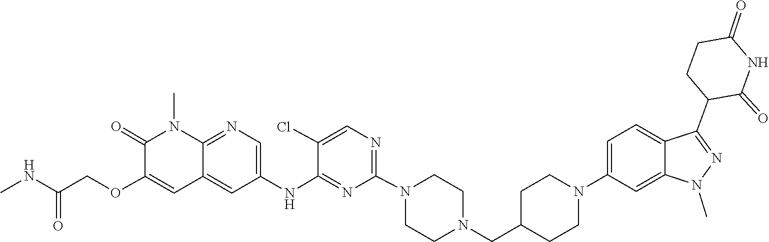 [SMILES: CNC(=O)COc1cc2cc(Nc3nc(N4CCN(CC5CCN(c6ccc7c(C8CCC(=O)NC8=O)nn(C)c7c6)CC5)CC4)ncc3Cl)cnc2n(C)c1=O]

### Preparation of 6-bromo-3-iodo-1-methyl-indazole

To a solution of 6-bromo-3-iodo-1H-indazole (30 g, 92.9 mmol, 1 eq.) in DMF (200 mL) was added NaH (7.43 g, 186 mmol, 60% purity, 2 eq.) at 0° C. The mixture was stirred for 1 hour. After stirring, Mel (26.4 g, 186 mmol, 11.6 mL, 2 eq.) was added into the solution. The mixture was stirred at 20° C. for 1 hour. After completion, the solution was cooled to 0° C., and saturated NH₄Cl solution (300 mL) was added dropwise. The solution was extracted with ethyl acetate (500 mL×3). The combined organic layers were washed with brine (500 mL), dried over anhydrous sodium sulfate, filtered, and concentrated under reduced pressure to give a residue. The residue was purified by reverse-phase HPLC (0.1% FA condition) to afford 6-bromo-3-iodo-1-methyl-indazole (15.3 g) as a brown solid.

LC-MS (ESI): m/z=336.8 [M+H]⁺.

### Preparation of 6-bromo-3-(2,6-dibenzyloxy-3-pyridyl)-1-methyl-indazole

A mixture of 6-bromo-3-iodo-1-methyl-indazole (14.8 g, 43.9 mmol, 1 eq.), 2,6-dibenzyloxy-3-(4,4,5,5-tetramethyl-1,3,2-dioxaborolan-2-yl)pyridine (18.3 g, 43.9 mmol, 1 eq.), Pd(dppf)Cl₂ (3.21 g, 4.39 mmol, 0.1 eq.), Cs2CO₃ (42.9 g, 131.8 mmol, 3 eq.) and H₂O (10 mL) in THF (100 mL) was degassed and purged with N₂ 3 times. The mixture was stirred at 60° C. for 16 hours under N₂ atmosphere. After completion, the reaction mixture was poured into water (200 mL) and extracted with ethyl acetate (150 mL×3). The combined organic layers were washed with brine (200 mL), dried over anhydrous sodium sulfate, filtered, and concentrated under reduced pressure to give a residue. The residue was purified by column chromatography (SiO₂, petroleum ether/ethyl acetate=100/1 to 20/1) to afford 6-bromo-3-(2,6-dibenzyloxy-3-pyridyl)-1-methyl-indazole (14 g) as a yellow solid.

¹H NMR (400 MHz, DMSO-d₆) δ=7.94 (d, J=1.2 Hz, 1H), 7.91 (d, J=8.0 Hz, 1H), 7.61 (d, J=8.4 Hz, 1H), 7.51-7.44 (m, 2H), 7.42-7.23 (m, 8H), 7.10 (dd, J=1.6, 8.8 Hz, 1H), 6.59 (d, J=8.0 Hz, 1H), 5.44 (d, J=8.4 Hz, 4H), 4.04 (s, 3H).

### Preparation of tert-butyl 4-((1-(3-(2,6-bis(benzyloxy)pyridin-3-yl)-1-methyl-1H-indazol-6-yl)piperidin-4-yl)methyl) piperazine-1-carboxylate

To a stirred suspension of 6-bromo-3-(2,6-dibenzyloxy-3-pyridyl)-1-methyl-indazole (1.0 g, 2.0 mmol) in 1,4-dioxane (15 mL) and DMF (15 mL) was added tert-butyl 4-(piperidin-4-ylmethyl) piperazine-1-carboxylate (0.68 g, 2.4 mmol) and cesium carbonate (1.95 g, 6.0 mmol). The reaction was purged with argon for 15 minutes; RuPhos (0.18 g, 0.4 mmol) and RuPhos-Pd-G₃ (0.08 g, 0.1 mmol) was added, and heated the reaction to 100° C. for 3 hours. The reaction mixture was diluted with water and extracted with ethyl acetate. The combined organic layers were washed with brine solution, dried over anhydrous Na₂SO₄, filtered, and concentrated to afford crude product. The crude was purified by flash column chromatography (amine silica, 35-60 mesh, 50% EtOAc in petroleum ether) to afford tert-butyl 4-((1-(3-(2,6-bis(benzyloxy)pyridin-3-yl)-1-methyl-1H-indazol-6-yl)piperidin-4-yl)methyl) piperazine-1-carboxylate (0.7 g) as a brown semi-solid.

LC-MS (ESI): m/z=703.72 [M+H]⁺.

### Preparation of tert-butyl 4-((1-(3-(2,6-dioxopiperidin-3-yl)-1-methyl-1H-indazol-6-yl)piperidin-4-yl)methyl) piperazine-1-carboxylate

To a stirred solution of tert-butyl 4-((1-(3-(2,6-bis(benzyloxy)pyridin-3-yl)-1-methyl-1H-indazol-6-yl)piperidin-4-yl)methyl) piperazine-1-carboxylate (0.70 g, 0.99 mmol) in mixture of isopropyl alcohol (10 mL), ethyl acetate (10 mL) and DMF (7 mL) was added 10% Pd/C (0.69 g, 6.50 mmol). The reaction was put under hydrogen atmosphere (balloon) for 12 hours at room temperature. The reaction mixture was filtered through a pad of celite using 50% THF-DCM. The filtrate was concentrated under reduced pressure to afford tert-butyl 4-((1-(3-(2,6-dioxopiperidin-3-yl)-1-methyl-1H-indazol-6-yl)piperidin-4-yl)methyl) piperazine-1-carboxylate (0.40 g) as a brown solid.

LC-MS (ESI): m/z=525.55 [M+H]⁺.

### Preparation of 3-(1-methyl-6-(4-(piperazin-1-ylmethyl)piperidin-1-yl)-1H-indazol-3-yl)piperidine-2,6-dione (HCl salt)

To a stirred solution of tert-butyl 4-((1-(3-(2,6-dioxopiperidin-3-yl)-1-methyl-1H-indazol-6-yl)piperidin-4-yl)methyl) piperazine-1-carboxylate (0.40 g, 0.76 mmol) in DCM (4 mL) was added 4 M HCl in dioxane (2 mL). The reaction was stirred at room temperature for 3 hours. The reaction mixture was concentrated under reduced pressure to afford 3-(1-methyl-6-(4-(piperazin-1-ylmethyl)piperidin-1-yl)-1H-indazol-3-yl)piperidine-2,6-dione (HCl salt) (0.40 g) as a brown solid.

LC-MS (ESI): m/z=425.40 [M+H]⁺.

### Preparation of 2-((6-((5-chloro-2-(4-((1-(3-(2,6-dioxopiperidin-3-yl)-1-methyl-1H-indazol-6-yl)piperidin-4-yl)methyl) piperazin-1-yl)pyrimidin-4-yl)amino)-1-methyl-2-oxo-1,2-dihydro-1,8-naphthyridin-3-yl)oxy)-N-methylacetamide

To a stirred solution of 3-(1-methyl-6-(4-(piperazin-1-ylmethyl)piperidin-1-yl)-1H-indazol-3-yl)piperidine-2,6-dione hydrochloride (0.21 g, 0.46 mmol), 2-((6-((2,5-dichloropyrimidin-4-yl)amino)-1-methyl-2-oxo-1,2-dihydro-1,8-naphthyridin-3-yl)oxy)-N-methylacetamide (0.09 g, 0.23 mmol) in DMSO (3 mL) was added N,N-diisopropylethylamine (1.2 mL, 6.99 mmol). The reaction was heated to 100° C. for 4 hours. Solvent was removed via centrifugal evaporator to obtain crude product. The crude product was purified by prep-HPLC to afford 2-((6-((5-chloro-2-(4-((1-(3-(2,6-dioxopiperidin-3-yl)-1-methyl-1H-indazol-6-yl)piperidin-4-yl)methyl) piperazin-1-yl)pyrimidin-4-yl)amino)-1-methyl-2-oxo-1,2-dihydro-1,8-naphthyridin-3-yl)oxy)-N-methylacetamide (26 mg) as a pale brown solid.

LC-MS (ESI): m/z=797.51 [M+H]⁺.

¹H NMR (400 MHz, DMSO-d₆): δ 10.84 (s, 1H), 9.09 (s, 1H), 8.70 (d, J=2.00 Hz, 1H), 8.35 (s, 1H), 8.31 (d, J=2.00 Hz, 1H), 7.94 (d, J=4.40 Hz, 1H), 7.47 (d, J=9.20 Hz, 1H), 7.18 (s, 1H), 6.90 (d, J=9.60 Hz, 1H), 6.82 (br s, 1H), 4.62 (s, 2H), 4.26-4.23 (m, 1H), 3.88 (s, 3H), 3.78 (d, J=12.00 Hz, 2H), 3.73 (s, 3H), 3.61-3.59 (m, 4H), 2.76-2.73 (m, 2H), 2.66 (d, J=4.80 Hz, 3H), 2.62-2.59 (m, 1H), 2.50-2.45 (m, 1H), 2.44-2.39 (m, 4H), 2.38-2.38 (m, 1H), 2.31-2.27 (m, 3H), 1.81-1.73 (m, 3H), 1.30-1.24 (m, 2H).

### Example 4. Synthesis of 2-((6-((5-chloro-2-(4-(2-(1-(3-(2,6-dioxopiperidin-3-yl)-1-methyl-1H-indazol-6-yl)piperidin-4-yl)ethyl) piperazin-1-yl)pyrimidin-4-yl)amino)-1-methyl-2-oxo-1,2-dihydro-1,8-naphthyridin-3-yl)oxy)-N-methylacetamide (Compound 114)

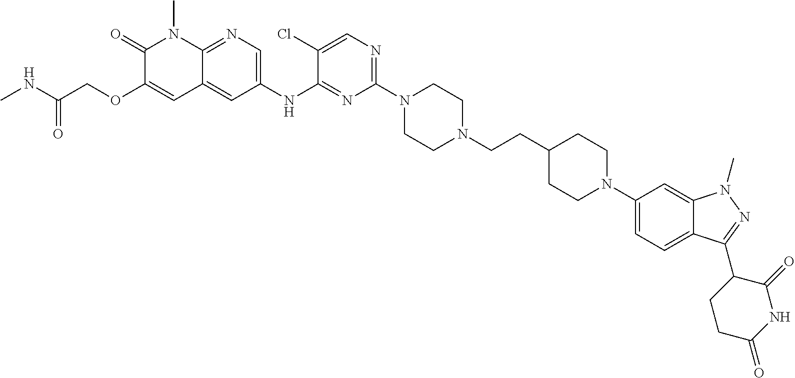 [SMILES: CNC(=O)COc1cc2cc(Nc3nc(N4CCN(CCC5CCN(c6ccc7c(C8CCC(=O)NC8=O)nn(C)c7c6)CC5)CC4)ncc3Cl)cnc2n(C)c1=O]

### Preparation of 2-(1-(3-(2,6-bis(benzyloxy)pyridin-3-yl)-1-methyl-1H-indazol-6-yl)piperidin-4-yl) ethan-1-ol

To a stirred suspension of 3-(2,6-bis(benzyloxy)pyridin-3-yl)-6-bromo-1-methyl-1H-indazole (2.5 g, 4.99 mmol) in 1,4-dioxane (50 mL) was added 2-(piperidin-4-yl) ethan-1-ol (0.97 g, 7.48 mmol) and cesium carbonate (4.87 g, 15.0 mmol). After purging with argon gas for 15 minutes, RuPhos (0.23 g, 0.5 mmol) and RuPhos-Pd-G3 (0.21 g, 0.25 mmol) were added. The reaction was heated at 100° C. for 5 hours. The reaction mixture was filtered through a celite bed and concentrated under reduced pressure. The crude was purified by column chromatography (SiO₂, 100-200 mesh, 65% EtOAc in petroleum ether) to afford 2-(1-(3-(2,6-bis(benzyloxy)pyridin-3-yl)-1-methyl-1H-indazol-6-yl)piperidin-4-yl) ethan-1-ol (0.75 g) as a yellow semi-solid.

LC-MS (ESI): m/z=549.63 [M+H]⁺.

### Preparation of 2-(1-(3-(2,6-bis(benzyloxy)pyridin-3-yl)-1-methyl-1H-indazol-6-yl)piperidin-4-yl) ethyl methanesulfonate

A stirred solution of 2-(1-(3-(2,6-bis(benzyloxy)pyridin-3-yl)-1-methyl-1H-indazol-6-yl)piperidin-4-yl) ethan-1-ol (0.75 g, 1.37 mmol) in DCM was cooled to 0° C. TEA (0.57 mL, 4.1 mmol) and MsCl (0.32 mL, 4.1 mmol) was added and the reaction was stirred for 2 hours. The reaction mixture was concentrated under reduced pressure to afford crude product. The crude product was washed with n-pentane to afford 2-(1-(3-(2,6-bis(benzyloxy)pyridin-3-yl)-1-methyl-1H-indazol-6-yl)piperidin-4-yl) ethyl methanesulfonate (1.0 g) as an orange semi-solid.

LC-MS (ESI): m/z=627.37 [M+H]⁺.

### Preparation of tert-butyl 4-(2-(1-(3-(2,6-bis(benzyloxy)pyridin-3-yl)-1-methyl-1H-indazol-6-yl)piperidin-4-yl)ethyl) piperazine-1-carboxylate

To a stirred solution of 2-(1-(3-(2,6-bis(benzyloxy)pyridin-3-yl)-1-methyl-1H-indazol-6-yl)piperidin-4-yl) ethyl methanesulfonate (0.7 g, 1.12 mmol) and tert-butyl piperazine-1-carboxylate (0.42 g, 2.24 mmol) in DMSO (5 mL) was added N,N-diisopropylethylamine (1.59 mL, 8.96 mmol). The reaction mixture was stirred at 50° C. for 16 hours. The reaction mixture was diluted with ice water (10 mL) and extracted with ethyl acetate (2×20 mL). The separated organic layers were washed with brine solution, dried over anhydrous Na₂SO₄, filtered, and concentrated to afford crude product. The crude product was purified by column chromatography (SiO₂, 100-200 mesh; 74% EtOAc in petroleum ether) to afford tert-butyl 4-(2-(1-(3-(2,6-bis(benzyloxy)pyridin-3-yl)-1-methyl-1H-indazol-6-yl)piperidin-4-yl)ethyl) piperazine-1-carboxylate (0.7 g) as an off-white solid.

LC-MS (ESI): m/z=717.75 [M+H]⁺.

### Preparation of tert-butyl 4-(2-(1-(3-(2,6-dioxopiperidin-3-yl)-1-methyl-1H-indazol-6-yl)piperidin-4-yl)ethyl) piperazine-1-carboxylate

To a stirred solution of tert-butyl 4-(2-(1-(3-(2,6-bis(benzyloxy)pyridin-3-yl)-1-methyl-1H-indazol-6-yl)piperidin-4-yl)ethyl) piperazine-1-carboxylate (0.70 g, 0.97 mmol) in THF (21 mL) was added 20% palladium hydroxide (0.90 g) and acetic acid (0.4 mL). The reaction was put under H₂ atmosphere (80 psi) and stirred at room temperature for 8 hours. The reaction mixture was filtered through a celite bed and concentrated to obtain crude product. The crude was washed with n-pentane and dried to obtain tert-butyl 4-(2-(1-(3-(2,6-dioxopiperidin-3-yl)-1-methyl-1H-indazol-6-yl)piperidin-4-yl)ethyl) piperazine-1-carboxylate (0.55 g) as a green semi-solid.

LC-MS (ESI): m/z=539.63 [M+H]⁺.

### Preparation of 3-(1-methyl-6-(4-(2-(piperazin-1-yl)ethyl)piperidin-1-yl)-1H-indazol-3-yl)piperidine-2,6-dione

A stirred solution of tert-butyl 4-(2-(1-(3-(2,6-dioxopiperidin-3-yl)-1-methyl-1H-indazol-6-yl)piperidin-4-yl)ethyl) piperazine-1-carboxylate (0.55 g, 1.02 mmol) in DCM (6 mL) was cooled to 0° C. TFA (5.5 mL) was added, and the reaction was allowed to warm to room temperature with stirring over 2 hours. The reaction mixture was concentrated under reduced pressure to afford crude product. The crude product was washed with n-pentane to obtain 3-(1-methyl-6-(4-(2-(piperazin-1-yl)ethyl)piperidin-1-yl)-1H-indazol-3-yl)piperidine-2,6-dione (0.3 g) as a brown solid.

LC-MS (ESI): m/z=439.55 [M+H]⁺.

### Preparation of 2-((6-((5-chloro-2-(4-(2-(1-(3-(2,6-dioxopiperidin-3-yl)-1-methyl-1H-indazol-6-yl)piperidin-4-yl)ethyl) piperazin-1-yl)pyrimidin-4-yl)amino)-1-methyl-2-oxo-1,2-dihydro-1,8-naphthyridin-3-yl)oxy)-N-methylacetamide

To a stirred solution of 3-(1-methyl-6-(4-(2-(piperazin-1-yl)ethyl)piperidin-1-yl)-1H-indazol-3-yl)piperidine-2,6-dione (0.15 g, 0.34 mmol) and 2-((6-((2,5-dichloropyrimidin-4-yl)amino)-1-methyl-2-oxo-1,2-dihydro-1,8-naphthyridin-3-yl)oxy)-N-methylacetamide (0.1 g, 0.21 mmol) in DMSO (3 mL) was added N, N-diisopropylethylamine (0.35 g, 2.72 mmol). The reaction mixture was stirred at 100° C. for 5 hours. Solvent was removed via centrifugal evaporator to afford crude product. The crude product was purified by prep-HPLC to afford 2-((6-((5-chloro-2-(4-(2-(1-(3-(2,6-dioxopiperidin-3-yl)-1-methyl-1H-indazol-6-yl)piperidin-4-yl)ethyl) piperazin-1-yl)pyrimidin-4-yl)amino)-1-methyl-2-oxo-1,2-dihydro-1,8-naphthyridin-3-yl)oxy)-N-methylacetamide (11 mg) as a pale brown solid.

LC-MS (ESI): m/z=811.51 [M+H]⁺.

¹H NMR (400 MHz, DMSO-d₆): δ 10.84 (s, 1H), 9.09 (s, 1H), 8.70 (d, J=2.40 Hz, 1H), 8.31 (d, J=2.40 Hz, 1H), 8.09 (s, 1H), 7.95 (dd, J=4.40, Hz, 1H), 7.47 (dd, J=8.80, Hz, 1H), 7.18 (s, 1H), 6.89 (dd, J=9.20, Hz, 1H), 6.82 (s, 1H), 4.62 (s, 2H), 4.24-4.22 (m, 1H), 3.88 (s, 3H), 3.7-3.73 (m, 5H), 3.60 (s, 3H), 2.62-2.60 (m, 7H), 2.33-2.30 (m, 8H), 2.14-2.13 (m, 1H), 1.79-1.75 (m, 2H), 1.68 (br s, 4H), 1.51-1.45 (m, 1H).

### Example 5. Synthesis of 2-((5-((5-chloro-2-(4-(2-(1-(3-(2,5-dioxopiperidin-3-yl)-1-methyl-1H-indazol-7-yl)piperidin-4-yl)ethyl)piperazin-1-yl)pyrimidin-4-yl)amino)-1-methyl-2-oxo-1,2-dihydro-1,8-naphthyridin-3-yl)oxy)-N-methylacetamide (Compound 115)

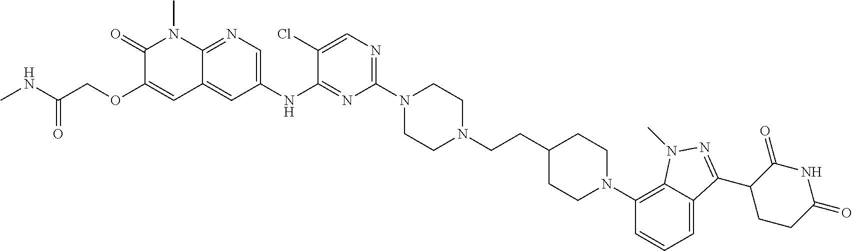 [SMILES: CNC(=O)COc1cc2cc(Nc3nc(N4CCN(CCC5CCN(c6cccc7c(C8CCC(=O)NC8=O)nn(C)c67)CC5)CC4)ncc3Cl)cnc2n(C)c1=O]

### Preparation of 2-(1-(3-(2,6-bis(benzyloxy)pyridin-3-yl)-1-methyl-1H-indazol-7-yl)piperidin-4-yl) ethan-1-ol

To a stirred suspension of 3-(2,6-bis(benzyloxy)pyridin-3-yl)-7-bromo-1-methyl-1H-indazole (2.0 g, 4.0 mmol) in 1,4-dioxane (50 mL) was added 2-(piperidin-4-yl) ethan-1-ol (0.77 g, 6.0 mmol) and cesium carbonate (3.91 g, 12.0 mmol). After purging with argon gas for 15 minutes, PEPPSI iHept C₁ (0.19 g, 0.20 mmol) was added, and the reaction was heated at 100° C. for 5 hours. The reaction mixture was filtered through a celite bed and concentrated under reduced pressure to afford crude product. The crude was purified by column chromatography (SiO₂, 100-200 mesh, 65% EtOAc in petroleum ether) to afford 2-(1-(3-(2,6-bis(benzyloxy)pyridin-3-yl)-1-methyl-1H-indazol-7-yl)piperidin-4-yl) ethan-1-ol (0.70 g) as a yellow solid. LC-MS (ESI): m/z=549.59 [M+H]⁺.

### Preparation of 2-(1-(3-(2,6-bis(benzyloxy)pyridin-3-yl)-1-methyl-1H-indazol-7-yl)piperidin-4-yl) ethyl methanesulfonate

A stirred solution of 2-(1-(3-(2,6-bis(benzyloxy)pyridin-3-yl)-1-methyl-1H-indazol-7-yl)piperidin-4-yl) ethan-1-ol (0.70 g, 1.27 mmol)) in DCM cooled to 0° C. TEA (0.53 mL, 3.81 mmol) and MsCl (0.30 mL, 3.81 mmol) were added, and the reaction was allowed to warm to room temperature over 2 hours. Th reaction mixture was concentrated under reduced pressure to afford crude product. The crude product was washed with n-pentane to afford 2-(1-(3-(2,6-bis(benzyloxy)pyridin-3-yl)-1-methyl-1H-indazol-7-yl)piperidin-4-yl) ethyl methanesulfonate (1.0 g) as an orange semi-solid.

LC-MS (ESI): m/z=627.57 [M+H]⁺.

### Preparation of tert-butyl 4-(2-(1-(3-(2,6-bis(benzyloxy)pyridin-3-yl)-1-methyl-1H-indazol-7-yl)piperidin-4-yl)ethyl) piperazine-1-carboxylate

To a stirred solution of 2-(1-(3-(2,6-bis(benzyloxy)pyridin-3-yl)-1-methyl-1H-indazol-7-yl)piperidin-4-yl) ethyl methanesulfonate (0.7 g, 1.12 mmol) and tert-butyl piperazine-1-carboxylate (0.42 g, 2.24 mmol) in DMSO was added N, N-diisopropylethylamine (1.59 mL, 8.96 mmol). The reaction mixture was then stirred at 50° C. for 16 hours. The reaction mixture was diluted with ice water (70 mL) and extracted with ethyl acetate (2×70 mL). The separated organic layers were washed with brine solution, dried over anhydrous Na₂SO₄, filtered, and concentrated to afford crude product. The crude product was purified by column chromatography (SiO₂, 100-200 mesh; 74% EtOAc in petroleum ether) to afford tert-butyl 4-(2-(1-(3-(2,6-bis(benzyloxy)pyridin-3-yl)-1-methyl-1H-indazol-7-yl)piperidin-4-yl)ethyl) piperazine-1-carboxylate (0.7 g) as an off-white solid.

### Preparation of tert-butyl 4-(2-(1-(3-(2,6-dioxopiperidin-3-yl)-1-methyl-1H-indazol-7-yl)piperidin-4-yl)ethyl) piperazine-1-carboxylate

A stirred solution of tert-butyl 4-(2-(1-(3-(2,6-bis(benzyloxy)pyridin-3-yl)-1-methyl-1H-indazol-7-yl)piperidin-4-yl)ethyl) piperazine-1-carboxylate (0.7 g, 0.97 mmol) in THF (21 mL) was degassed with nitrogen and then 20% palladium hydroxide (0.90 g) was added. The reaction mixture was put under H₂ atmosphere (80 psi) and stirred at room temperature for 16 hours. The reaction mixture was filtered through a celite bed and concentrated to obtain crude product. The crude product was washed with n-pentane and dried to afford tert-butyl 4-(2-(1-(3-(2,6-dioxopiperidin-3-yl)-1-methyl-1H-indazol-7-yl)piperidin-4-yl)ethyl) piperazine-1-carboxylate (0.55 g) as a green solid.

LC-MS (ESI): m/z=539.63 [M+H]⁺.

### Preparation of 3-(1-methyl-7-(4-(2-(piperazin-1-yl)ethyl)piperidin-1-yl)-1H-indazol-3-yl)piperidine-2,6-dione

A stirred solution of tert-butyl 4-(2-(1-(3-(2,6-dioxopiperidin-3-yl)-1-methyl-1H-indazol-7-yl)piperidin-4-yl)ethyl) piperazine-1-carboxylate (0.50 g, 0.92 mmol) in DCM (5.0 mL) was cooled to 0° C. and TFA (5.0 mL) was added. The reaction mixture was allowed to warm to room temperature with stirring over 2 hours. The reaction mixture was concentrated under reduced pressure to remove solvent. The crude product was washed with n-pentane to obtain 3-(1-methyl-7-(4-(2-(piperazin-1-yl)ethyl)piperidin-1-yl)-1H-indazol-3-yl)piperidine-2,6-dione (0.3 g) as a brown solid.

LC-MS (ESI): m/z=439.55 [M+H]⁺.

### Preparation of 2-((6-((5-chloro-2-(4-(2-(1-(3-(2,6-dioxopiperidin-3-yl)-1-methyl-1H-indazol-7-yl)piperidin-4-yl)ethyl) piperazin-1-yl)pyrimidin-4-yl)amino)-1-methyl-2-oxo-1,2-dihydro-1,8-naphthyridin-3-yl)oxy)-N-methylacetamide

To a stirred solution of 3-(1-methyl-7-(4-(2-(piperazin-1-yl)ethyl)piperidin-1-yl)-1H-indazol-3-yl)piperidine-2,6-dione (0.15 g, 0.34 mmol) and 2-((6-((2,5-dichloropyrimidin-4-yl)amino)-1-methyl-2-oxo-1,2-dihydro-1,8-naphthyridin-3-yl)oxy)-N-methylacetamide (0.08 g, 0.20 mmol) in DMSO (3 mL) was added N, N-diisopropylethylamine (0.35 g, 2.72 mmol). The reaction mixture was stirred at 100° C. for 5 hours. The reaction mixture was poured into ice-cold water and stirred for 15 minutes. The resulting precipitate was filtered and dried to afford crude product. The crude product was purified by prep-HPLC to afford 2-((6-((5-chloro-2-(4-(2-(1-(3-(2,6-dioxopiperidin-3-yl)-1-methyl-1H-indazol-7-yl)piperidin-4-yl)ethyl) piperazin-1-yl)pyrimidin-4-yl)amino)-1-methyl-2-oxo-1,2-dihydro-1,8-naphthyridin-3-yl)oxy)-N-methylacetamide (0.14 g) as an off-white solid.

LC-MS (ESI): m/z=809.45 [M−H].

¹H NMR (400 MHz, DMSO-d₆): δ 10.87 (s, 1H), 9.09 (s, 1H), 8.70 (d, J=2.40 Hz, 1H), 8.31 (d, J=2.40 Hz, 1H), 8.09 (s, 1H), 7.98 (s, 2H), 7.94 (d, J=4.40 Hz, 1H), 7.36-7.35 (m, 1H), 7.19 (s, 1H), 7.00 (d, J=4.40 Hz, 2H), 6.88 (d, J=8.40 Hz, 2H), 4.62 (s, 2H), 4.33 (q, J=5.20 Hz, 1H), 4.23 (s, 3H), 3.74 (s, 3H), 3.22 (br, 2H), 3.61 (s, 4H), 2.61-2.58 (m, 7H), 2.31-2.29 (m, 8H), 2.18-2.17 (m, 1H), 1.84-1.81 (m, 2H).

### Example 6. Synthesis of 2-((6-((5-chloro-2-(4-((4-(3-(2,6-dioxopiperidin-3-yl)-1-methyl-1H-indazol-7-yl) piperazin-1-yl)methyl)piperidin-1-yl)pyrimidin-4-yl)amino)-1-methyl-2-oxo-1,2-dihydro-1,8-naphthyridin-3-yl)oxy)-N-methylacetamide (Compound 132)

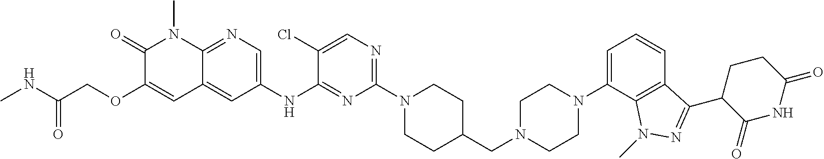 [SMILES: CNC(=O)COc1cc2cc(Nc3nc(N4CCC(CN5CCN(c6cccc7c(C8CCC(=O)NC8=O)nn(C)c67)CC5)CC4)ncc3Cl)cnc2n(C)c1=O]

### Preparation of benzyl 4-((1-(tert-butoxycarbonyl)piperidin-4-yl)methyl) piperazine-1-carboxylate

To a solution of benzyl piperazine-1-carboxylate (10.0 g, 45.39 mmol), tert-butyl 4-formylpiperidine-1-carboxylate (11.61 g, 54.47 mmol), and NaBH (OAc)₃ (9.62 g, 45.39 mmol) in DCM (200 mL) was added acetic acid (2.72 g, 45.39 mmol). The reaction was stirred at room temperature for 16 hours. The reaction mixture was quenched with ice-cold water (500 mL) and extracted into DCM (3×250 mL). The combined organic layers were washed with brine solution (500 mL) and dried over anhydrous Na₂SO₄, filtered, and dried under vacuum to get crude product. The crude product was purified by column chromatography (SiO₂; 40% EtOAc in petroleum ether) to afford benzyl 4-((1-(tert-butoxycarbonyl)piperidin-4-yl) methyl) piperazine-1-carboxylate (6.0 g) as colorless liquid.

LC-MS (ESI): m/z=418.31. [M+H]⁺.

### Preparation of tert-butyl 4-(piperazin-1-ylmethyl)piperidine-1-carboxylate

To a stirred solution of benzyl 4-((1-(tert-butoxycarbonyl)piperidin-4-yl)methyl) piperazine-1-carboxylate (6.0 g, 14.36 mmol) in THF (180 mL) was added 20% Pd (OH)₂ on carbon (3.0 g). The reaction was put under a hydrogen atmosphere (60 psi) at room temperature and stirred for 16 hours. The reaction mixture was filtered through a celite bed and concentrated to obtain the crude product. The crude product was washed with n-pentane and dried to afford tert-butyl 4-(piperazin-1-ylmethyl)piperidine-1-carboxylate (3.0 g) as colorless liquid.

LC-MS (ESI): m/z=284.16 [M+H]⁺.

### Preparation of tert-butyl 4-((4-(3-(2,6-bis(benzyloxy)pyridin-3-yl)-1-methyl-1H-indazol-7-yl) piperazin-1-yl)methyl)piperidine-1-carboxylate

A solution of 3-(2,6-bis(benzyloxy)pyridin-3-yl)-7-bromo-1-methyl-1H-indazole (1.0 g, 2.0 mmol), tert-butyl 4-(piperazin-1-ylmethyl)piperidine-1-carboxylate (0.56 g, 2.0 mmol) and cesium carbonate (1.95 g, 6.0 mmol) in 1,4-dioxane (20 mL) was degassed with argon for 5 minutes. Pd-PEPPSI-iHeptCl (0.97 g, 0.1 mmol) was added, the reaction was flushed with argon for 5 minutes, and the reaction mixture was heated at 80° C. for 16 hours. The reaction mixture was quenched with ice-cold water (50 mL) and extracted into ethyl acetate (3×100 mL). The combined organic layers were washed with brine solution (50 mL), dried over anhydrous Na₂SO₄, filtered, and concentrated to afford crude product. The crude product was purified by column chromatography (SiO₂; 40% EtOAc in petroleum ether) to afford tert-butyl 4-((4-(3-(2,6-bis(benzyloxy)pyridin-3-yl)-1-methyl-1H-indazol-7-yl) piperazin-1-yl)methyl)piperidine-1-carboxylate (1.0 g) as a pale brown semi-solid. LC-MS (ESI): m/z=703.73 [M+H]⁺.

### Preparation of tert-butyl 4-((4-(3-(2,6-dioxopiperidin-3-yl)-1-methyl-1H-indazol-7-yl) piperazin-1-yl)methyl)piperidine-1-carboxylate

To a stirred solution of tert-butyl 4-((4-(3-(2,6-bis(benzyloxy)pyridin-3-yl)-1-methyl-1H-indazol-7-yl) piperazin-1-yl)methyl)piperidine-1-carboxylate (1.0 g, 1.42 mmol) in THF (60 mL) was added 20% Pd (OH)₂ on carbon (1.0 g). The reaction was put under hydrogen atmosphere (60 psi) and stirred at room temperature for 6 hours. The reaction mixture was filtered through a celite bed and concentrated to obtain crude product. The crude was washed with n-pentane and dried to afford tert-butyl 4-((4-(3-(2,6-dioxopiperidin-3-yl)-1-methyl-1H-indazol-7-yl) piperazin-1-yl)methyl)piperidine-1-carboxylate (0.70 g) as a brown solid. LC-MS (ESI): m/z=525.66 [M+H]⁺.

### Preparation of 3-(1-methyl-7-(4-(piperidin-4-ylmethyl) piperazin-1-yl)-1H-indazol-3-yl)piperidine-2,6-dione

A stirred solution of tert-butyl 4-((4-(3-(2,6-dioxopiperidin-3-yl)-1-methyl-1H-indazol-7-yl) piperazin-1-yl)methyl)piperidine-1-carboxylate (0.70 g, 1.33 mmol) in DCM (14 mL) was cooled to 0° C. TFA (7 mL) was added, and the reaction was allowed to warm to room temperature for 3 hours with stirring. The reaction mixture was concentrated to obtain crude product. The crude product was washed with n-pentane to afford 3-(1-methyl-7-(4-(piperidin-4-ylmethyl) piperazin-1-yl)-1H-indazol-3-yl)piperidine-2,6-dione (0.50 g, quantitative) as a brown gummy solid.

LC-MS (ESI): m/z=425.54 [M+H]⁺.

### Preparation of 2-((6-((5-chloro-2-(4-((4-(3-(2,6-dioxopiperidin-3-yl)-1-methyl-1H-indazol-7-yl) piperazin-1-yl)methyl)piperidin-1-yl)pyrimidin-4-yl)amino)-1-methyl-2-oxo-1,2-dihydro-1,8-naphthyridin-3-yl)oxy)-N-methylacetamide

To a stirred solution of 3-(1-methyl-7-(4-(piperidin-4-ylmethyl) piperazin-1-yl)-1H-indazol-3-yl)piperidine-2,6-dione (0.13 g, 0.30 mmol) and 2-((6-((2,5-dichloropyrimidin-4-yl)amino)-1-methyl-2-oxo-1,2-dihydro-1,8-naphthyridin-3-yl)oxy)-N-methylacetamide (0.08 g, 0.21 mmol) in DMSO was added N,N-diisopropylethylamine (0.31 g, 2.44 mmol). The reaction mixture was stirred at 100° C. for 16 hours. The reaction mixture was poured into ice-cold water and stirred for 15 minutes. The resulting precipitate was filtered and dried to obtain crude product. The crude product was purified by prep-HPLC to afford 2-((6-((5-chloro-2-(4-((4-(3-(2,6-dioxopiperidin-3-yl)-1-methyl-1H-indazol-7-yl) piperazin-1-yl)methyl)piperidin-1-yl)pyrimidin-4-yl)amino)-1-methyl-2-oxo-1,2-dihydro-1,8-naphthyridin-3-yl)oxy)-N-methylacetamide (0.018 g) as an off-white solid.

LC-MS (ESI): m/z=797.52 [M+H]⁺.

¹H NMR (400 MHz, DMSO-d₆): δ 10.92 (s, 1H), 8.77 (s, 1H), 8.18 (d, J=2.00 Hz, 1H), 8.03 s, 1H), 7.76-7.73 (m, 1H), 7.45 (d, J=9.20 Hz, 1H), 7.39 (t, J=4.80 Hz, 1H), 7.03-7.02 (m, 1H), 6.17 (br s, 1H), 4.61 (s, 2H), 4.47-4.44 (m, 2H), 4.35-4.31 (m, 1H), 4.25 (s, 3H), 3.57 (s, 3H), 3.20-2.75 (m, 14H), 2.33-2.17 (m, 6H), 1.75-1.72 (m, 3H), 1.04-1.02 (m, 2H).

### Example 7. Synthesis of 2-((6-((5-chloro-2-(4-((4-(3-(2,6-dioxopiperidin-3-yl)-1-methyl-1H-indazol-6-yl) piperazin-1-yl)methyl)piperidin-1-yl)pyrimidin-4-yl)amino)-1-methyl-2-oxo-1,2-dihydro-1,8-naphthyridin-3-yl)oxy)-N-methylacetamide (Compound 133)

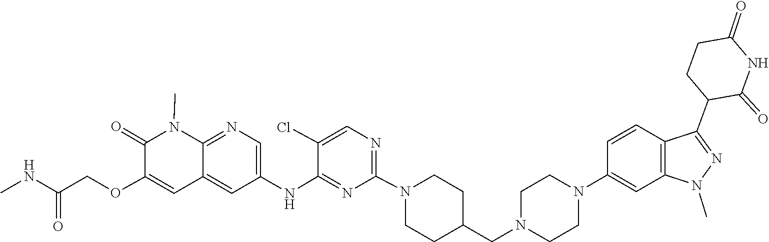 [SMILES: CNC(=O)COc1cc2cc(Nc3nc(N4CCC(CN5CCN(c6ccc7c(C8CCC(=O)NC8=O)nn(C)c7c6)CC5)CC4)ncc3Cl)cnc2n(C)c1=O]

### Preparation of tert-butyl 4-((4-(3-(2,6-bis(benzyloxy)pyridin-3-yl)-1-methyl-1H-indazol-6-yl) piperazin-1-yl)methyl)piperidine-1-carboxylate

A solution of 3-(2,6-bis(benzyloxy)pyridin-3-yl)-6-bromo-1-methyl-1H-indazole (1 g, 1.99 mmol), tert-butyl 4-(piperazin-1-ylmethyl)piperidine-1-carboxylate (1.13 g, 3.99 mmol), and cesium carbonate (1.94 g, 5.97 mmol) in 1,4-dioxane (20 mL) was degassed with argon for 5 minutes. Ruphos-Pd-G3 (0.07 g, 0.09 mmol) and Ruphos (0.08 g, 0.19 mmol) were added, the reaction was degassed for 5 minutes, and the reaction mixture was heated at 100° C. for 16 hours. The reaction mixture was quenched with ice-cold water (40 mL) and extracted into ethyl acetate (3×100 mL). The combined organic layers were washed with brine solution (40 mL), dried over anhydrous Na₂SO₄, filtered, and concentrated to afford crude product. The crude product was purified by column chromatography (SiO₂; 50% EtOAc in petroleum ether) to afford tert-butyl 4-((4-(3-(2,6-bis(benzyloxy)pyridin-3-yl)-1-methyl-1H-indazol-6-yl) piperazin-1-yl)methyl)piperidine-1-carboxylate (1.0 g) as a pale brown semi-solid. LC-MS (ESI): m/z=703.84 [M+H]⁺.

### Preparation of tert-butyl 4-((4-(3-(2,6-dioxopiperidin-3-yl)-1-methyl-1H-indazol-6-yl) piperazin-1-yl)methyl)piperidine-1-carboxylate

To a stirred solution of tert-butyl 4-((4-(3-(2,6-bis(benzyloxy)pyridin-3-yl)-1-methyl-1H-indazol-6-yl) piperazin-1-yl)methyl)piperidine-1-carboxylate (0.90 g, 1.28 mmol) in THF (54 mL) was added 20% Pd (OH)₂ on carbon (0.9 g). The reaction was put under hydrogen atmosphere (60 psi) and stirred at room temperature for 6 hours. The reaction mixture was filtered through a celite bed, and the filtrate was concentrated to afford crude product. The crude was washed with n-pentane and dried to afford tert-butyl 4-((4-(3-(2,6-dioxopiperidin-3-yl)-1-methyl-1H-indazol-6-yl) piperazin-1-yl)methyl)piperidine-1-carboxylate (0.7 g) as a brown solid.

LC-MS (ESI): m/z=525.65 [M+H]⁺.

### Preparation of 3-(1-methyl-6-(4-(piperidin-4-ylmethyl) piperazin-1-yl)-1H-indazol-3-yl)piperidine-2,6-dione

A stirred solution of tert-butyl 4-((4-(3-(2,6-dioxopiperidin-3-yl)-1-methyl-1H-indazol-6-yl) piperazin-1-yl)methyl)piperidine-1-carboxylate (0.7 g, 1.33 mmol) in DCM (14 mL) was cooled to 0° C. TFA (7.0 mL) was added, and the reaction mixture was allowed to warm to room temperature over 3 hours with stirring. The reaction mixture was concentrated to obtain crude product. The crude product was washed with n-pentane to afford 3-(1-methyl-6-(4-(piperidin-4-ylmethyl) piperazin-1-yl)-1H-indazol-3-yl)piperidine-2,6-dione (0.55 g) as a brown gummy solid. LC-MS (ESI): m/z=425.54 [M+H]⁺.

### Preparation of 2-((6-((5-chloro-2-(4-((4-(3-(2,6-dioxopiperidin-3-yl)-1-methyl-1H-indazol-6-yl) piperazin-1-yl)methyl)piperidin-1-yl)pyrimidin-4-yl)amino)-1-methyl-2-oxo-1,2-dihydro-1,8-naphthyridin-3-yl)oxy)-N-methylacetamide

To a stirred solution of 3-(1-methyl-6-(4-(piperidin-4-ylmethyl) piperazin-1-yl)-1H-indazol-3-yl)piperidine-2,6-dione (0.150 g, 0.35 mmol) and 2-((6-((2,5-dichloropyrimidin-4-yl)amino)-1-methyl-2-oxo-1,2-dihydro-1,8-naphthyridin-3-yl)oxy)-N-methylacetamide (0.14 g, 0.28 mmol) in DMSO (3 mL) was added N,N-diisopropylethylamine (0.36 g, 2.80 mmol). The reaction mixture was stirred at 100° C. for 16 hours. The reaction mixture was poured into ice-cold water and stirred for 15 minutes. The resulting precipitate was filtered and dried to afford crude product. The crude product was purified by prep-HPLC to afford 2-((6-((5-chloro-2-(4-((4-(3-(2,6-dioxopiperidin-3-yl)-1-methyl-1H-indazol-6-yl) piperazin-1-yl)methyl)piperidin-1-yl)pyrimidin-4-yl)amino)-1-methyl-2-oxo-1,2-dihydro-1,8-naphthyridin-3-yl)oxy)-N-methylacetamide (0.012 g) as a brown solid.

LC-MS (ESI): m/z=797.49 [M+H]⁺.

¹H NMR (400 MHz, DMSO-d₆): δ 10.85 (s, 1H), 9.04 (s, 1H), 8.73 (d, J=2.40 Hz, 1H), 8.47 (s, 1H), 8.29 (d, J=2.40 Hz, 1H), 8.07 (s, 1H), 7.95 (d, J=4.80 Hz, 1H), 7.49 (d, J=8.80 Hz, 1H), 7.16 (s, 1H), 6.91 (d, J=9.20 Hz, 1H), 4.61 (s, 2H), 4.45 (d, J=11.20 Hz, 2H), 4.25 (q, J=5.20 Hz, 1H), 3.89 (s, 3H), 3.73 (s, 3H), 3.27 (d, J=48.80 Hz, 4H), 2.85 (t, J=11.60 Hz, 2H), 2.65-2.62 (m, 9H), 2.33-2.32 (m, 1H), 2.21-2.19 (m, 3H), 1.78 (t, J=12.80 Hz, 3H), 1.08-1.04 (m, 2H).

### Example 8. Synthesis of 2-((6-((5-chloro-2-((3S)-3-(((3-(2,6-dioxopiperidin-3-yl)-1-methyl-1H-indazol-6-yl)amino)methyl) pyrrolidin-1-yl)pyrimidin-4-yl)amino)-1-methyl-2-oxo-1,2-dihydro-1,8-naphthyridin-3-yl)oxy)-N-methylacetamide (Compound 123b)

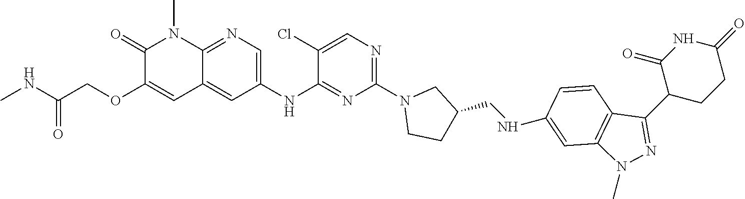 [SMILES: CNC(=O)COc1cc2cc(Nc3nc(N4CC[C@@H](CNc5ccc6c(C7CCC(=O)NC7=O)nn(C)c6c5)C4)ncc3Cl)cnc2n(C)c1=O]

### Preparation of tert-butyl(S)-3-(((3-(2,6-bis(benzyloxy)pyridin-3-yl)-1-methyl-1H-indazol-6-yl)amino)methyl) pyrrolidine-1-carboxylate

A solution of tert-butyl(S)-3-(aminomethyl) pyrrolidine-1-carboxylate (0.3 g, 1.49 mmol), 3-(2,6-bis(benzyloxy)pyridin-3-yl)-6-bromo-1-methyl-1H-indazole (0.75 g, 1.49 mmol), and potassium tert-butoxide (0.5 g, 4.49 mmol) in 1,4-dioxane (6 mL) was degassed with nitrogen for 15 minutes. tBuBrettPhos Pd G3 (0.06 g, 0.07 mmol) was added, the mixture was degassed again for 10 minutes, and the reaction was stirred at room temperature for 2 hours. The reaction mixture was filtered through a celite bed, and the filtrate was concentrated under reduced pressure to afford crude product. The crude product was purified by flash column chromatography (SiO₂, 100-200 mesh, 25% ethyl acetate in petroleum ether) to afford tert-butyl(S)-3-(((3-(2,6-bis(benzyloxy)pyridin-3-yl)-1-methyl-1H-indazol-6-yl)amino)methyl) pyrrolidine-1-carboxylate (0.6 g) as a brown solid.

LC-MS (ESI): m/z=620.66 [M+H]⁺.

### Preparation of tert-butyl (3S)-3-(((3-(2,6-dioxopiperidin-3-yl)-1-methyl-1H-indazol-6-yl)amino)methyl) pyrrolidine-1-carboxylate

To stirred solution of tert-butyl(S)-3-(((3-(2,6-bis(benzyloxy)pyridin-3-yl)-1-methyl-1H-indazol-6-yl)amino)methyl) pyrrolidine-1-carboxylate (0.6 g, 0.96 mmol) in tetrahydrofuran (36 mL) was added palladium hydroxide (0.54 g). The reaction mixture was put under hydrogen atmosphere (15 psi) and stirred at room temperature for 3 hours. The reaction mixture was filtered through a celite bed and washed, and the filtrate was concentrated under reduced pressure to afford crude product. The crude product was purified by flash column chromatography (SiO₂, 100-200 mesh, 60% Ethyl acetate in Pet ether) to afford tert-butyl (3S)-3-(((3-(2,6-dioxopiperidin-3-yl)-1-methyl-1H-indazol-6-yl)amino)methyl) pyrrolidine-1-carboxylate (0.3 g) as a brown semi-solid. LC-MS (ESI): m/z=442.45 [M+H]⁺.

### Preparation of 3-(1-methyl-6-((((R)-pyrrolidin-3-yl)methyl) amino)-1H-indazol-3-yl)piperidine-2,6-dione hydrochloride

To a stirred suspension of tert-butyl (3S)-3-(((3-(2,6-dioxopiperidin-3-yl)-1-methyl-1H-indazol-6-yl)amino)methyl) pyrrolidine-1-carboxylate (0.3 g, 0.67 mmol) in DCM (6 mL), hydrochloric acid was added (3 mL) at 0° C. The reaction mixture was allowed to warm to room temperature over 3 hours with stirring. The reaction mixture concentrated under reduced pressure to afford crude product. The crude product was purified by trituration to afford 3-(1-methyl-6-((((R)-pyrrolidin-3-yl)methyl) amino)-1H-indazol-3-yl)piperidine-2,6-dione hydrochloride (0.2 g) as a white solid.

LC-MS (ESI): m/z=342.40 [M+H]⁺.

### Preparation of 2-((6-((5-chloro-2-((3S)-3-(((3-(2,6-dioxopiperidin-3-yl)-1-methyl-1H-indazol-6-yl)amino)methyl) pyrrolidin-1-yl)pyrimidin-4-yl)amino)-1-methyl-2-oxo-1,2-dihydro-1,8-naphthyridin-3-yl)oxy)-N-methylacetamide

To a stirred solution of 3-(1-methyl-6-((((R)-pyrrolidin-3-yl)methyl) amino)-1H-indazol-3-yl)piperidine-2,6-dione hydrochloride (0.18 g, 0.47 mmol) and 2-((6-((2,5-dichloropyrimidin-4-yl)amino)-1-methyl-2-oxo-1,2-dihydro-1,8-naphthyridin-3-yl)oxy)-N-methylacetamide (0.08 g, 0.19 mmol) in dimethyl sulfoxide (3.6 mL) was added N,N-diisopropylethylamine (0.4 mL, 2.38 mmol) at room temperature. The resulting mixture was stirred at 100° C. for 4 hours. After cooling to room temperature water was added, and the reaction mixture was stirred for 15 minutes. The precipitate was filtered and dried to afford crude product. The crude product was purified by prep HPLC to afford 2-((6-((5-chloro-2-((3S)-3-(((3-(2,6-dioxopiperidin-3-yl)-1-methyl-1H-indazol-6-yl)amino)methyl) pyrrolidin-1-yl)pyrimidin-4-yl)amino)-1-methyl-2-oxo-1,2-dihydro-1,8-naphthyridin-3-yl)oxy)-N-methylacetamide as an off-white solid (0.029 g).

LC-MS (ESI): m/z=714.38 [M+H]⁺.

¹H NMR (400 MHz, DMSO-d₆): δ 10.81 (s, 1H), 8 8.98 (s, 1H), 8.84 (s, 1H), 8.43 (bs, 1H), 8.07 (s, 1H), 7.93 (bs, 1H), 7.32 (d, J=8.40 Hz, 1H), 7.21 (s, 1H), 6.55 (d, J=8.40 Hz, 1H), 6.38 (s, 1H), 6.07 (s, 1H), 4.60 (s, 2H), 4.17-4.16 (m, 1H), 3.79 (s, 3H), 3.73 (s, 4H), 3.57 (br, 1H), 3.42 (br, 1H), 3.14 (br, 3H), 2.65-2.59 (m, 6H), 2.11-2.13 (m, 3H), 1.78-1.76 (m, 1H).

### Example 9. Synthesis of 2-((6-((5-chloro-2-(4-((1-(3-(2,6-dioxopiperidin-3-yl)-1-methyl-1H-indazol-7-yl)piperidin-4-yl)methyl) piperazin-1-yl)pyrimidin-4-yl)amino)-1-isopropyl-2-oxo-1,2-dihydro-1,8-naphthyridin-3-yl)oxy)-N-methylacetamide (Compound 103)

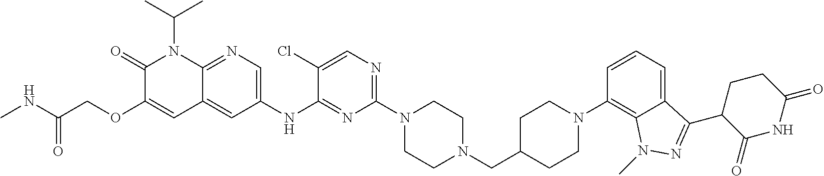 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)cnc2n(C(C)C)c1=O]

### Preparation of 1-isopropyl-5-nitro-1H-pyrrolo[2,3-b]pyridine

To a solution of 5-nitro-1H-pyrrolo[2,3-b]pyridine (10.0 g, 61.34 mmol) and potassium carbonate (12.6 g, 92.0 mmol) in DMF (200 mL) was added isopropyliodide (7.0 mL, 73.6 mmol) dropwise over a period of 20 minutes. The reaction was stirred at room temperature for 12 hours. The reaction mixture was quenched with ice-cold water (500 mL). The precipitated solid was filtered and dried under vacuum to afford 1-isopropyl-5-nitro-1H-pyrrolo[2,3-b] pyridine (10.9 g) as a pale yellow solid.

LC-MS (ESI): m/z=206.20 [M+H]⁺.

### Preparation of 1-isopropyl-5-nitro-1H-pyrrolo[2,3-b]pyridine-2,3-dione

To a stirred solution of 1-isopropyl-5-nitro-1H-pyrrolo[2,3-b]pyridine (10.9 g, 53.2 mmol) in acetone (76 mL) was added slowly a solution of CrO₃ (68.4 g, 691 mmol) in acetic acid (348 mL)/H₂O (109 mL) at 0° C. The reaction was allowed to warm to room temperature over 1 hour with stirring. The reaction mixture was diluted with water (500 mL) and extracted with DCM (400 mL×2). The separated organic layers were washed with brine solution, dried over Na₂SO₄, filtered, and concentrated to afford 1-isopropyl-5-nitro-1H-pyrrolo[2,3-b]pyridine-2,3-dione (9.64 g) as a pale yellow solid.

LC-MS (ESI): m/z=254.28 [M+18]⁺.

### Preparation of 1-isopropyl-3-methoxy-6-nitro-1,8-naphthyridin-2 (1H)-one

To a cooled solution of 1-isopropyl-5-nitro-1H-pyrrolo[2,3-b]pyridine-2,3-dione (9.64 g, 41.0 mmol) in EtOH (192 mL) was added triethyl amine (9.25 mL, 103 mmol) and trimethylsilyl diazomethane (2.0 M in diethyl ether) (9.82 g, 86.1 mmol) at 0° C. The reaction was allowed to warm to room temperature over 4 hours with stirring. The reaction mixture was quenched with water (400 mL) and extracted with ethyl acetate (300 mL×3). The combined organic layers were washed with brine solution, dried over Na₂SO₄, filtered, and concentrated to afford 1-isopropyl-3-methoxy-6-nitro-1,8-naphthyridin-2 (1//)-one (3.20 g) as a brown semi-solid. LC-MS (ESI): m/z=264.07 [M+H]⁺.

### Preparation of 3-hydroxy-1-isopropyl-6-nitro-1,8-naphthyridin-2 (1H)-one

To a cooled solution of 1-isopropyl-3-methoxy-6-nitro-1,8-naphthyridin-2 (1H)-one (3.20 g, 12.16 mmol) in DCM (64 mL) was added BBr₃ in DCM (32 mL) dropwise at 0° C. The reaction was stirred at 0° C. for 4 hours. The reaction mixture was quenched with methanol at 0° C. The reaction was concentrated under reduced pressure to obtain crude product. The crude product was triturated with diethyl ether to afford 3-hydroxy-1-isopropyl-6-nitro-1,8-naphthyridin-2 (1H)-one (2.2 g) as brown solid.

LC-MS (ESI): m/z=250.26 [M+H]⁺

### Preparation of 2-((1-isopropyl-6-nitro-2-oxo-1,2-dihydro-1,8-naphthyridin-3-yl)oxy)-N-methylacetamide

To a stirred solution of 3-hydroxy-1-isopropyl-6-nitro-1,8-naphthyridin-2 (1H)-one (2.20 g, 8.83 mmol) in DMF (44 mL) was added cesium carbonate (8.61 g, 26.5 mmol) and 2-bromo-N-methylacetamide (1.59 g, 10.6 mmol). The reaction was stirred at room temperature for 3 hours. The reaction was quenched with ice-cold water (300 mL) and extracted with 10% MeOH in DCM (200 mL×2). The separated organic layers were washed with brine solution, dried over Na₂SO₄ filtered, and concentrated to afford 2-((1-isopropyl-6-nitro-2-oxo-1,2-dihydro-1,8-naphthyridin-3-yl)oxy)-N-methylacetamide (2.10 g) as a brown solid.

LC-MS (ESI): m/z=321.31 [M+H]⁺.

### Preparation of 2-((6-amino-1-isopropyl-2-oxo-1,2-dihydro-1,8-naphthyridin-3-yl)oxy)-N-methylacetamide

To a solution of 2-((1-isopropyl-6-nitro-2-oxo-1,2-dihydro-1,8-naphthyridin-3-yl)oxy)-N-methylacetamide (2.10 g, 6.56 mmol) in MeOH/H₂O (63/63 mL) was added Fe (3.60 g, 65.6 mmol) and ammonium chloride (3.47 mL, 65.6 mmol). The reaction was stirred at 75° C. for 2 hours. The reaction mixture was diluted with DCM (300 mL) and filtered through a pad of a celite. The collected filtrate was concentrated under reduced pressure to obtain crude product. The crude product was triturated with n-pentane to afford 2-((6-amino-1-isopropyl-2-oxo-1,2-dihydro-1,8-naphthyridin-3-yl)oxy)-N-methylacetamide (1.49 g) as a grey solid. LC-MS (ESI): m/z=291.35 [M+H]⁺.

### Preparation of 2-((6-((2,5-dichloropyrimidin-4-yl)amino)-1-isopropyl-2-oxo-1,2-dihydro-1,8-naphthyridin-3-yl)oxy)-N-methylacetamide

In a vial equipped with stir bar, a solution of 2-((6-amino-1-isopropyl-2-oxo-1,2-dihydro-1,8-naphthyridin-3-yl)oxy)-N-methylacetamide (500 mg 1.72 mmol) and DIPEA (490 mg, 660 μL, 3.79 mmol) in DMSO (8.5 mL) was treated with 2,4,5-trichloropyrimidine (347 mg, 1.1 eq., 1.89 mmol). The resulting solution was stirred at room temperature for 18 hours. The reaction mixture was diluted with water (30 mL). The precipitate was collected by filtration and washed with water (5 mL). The precipitate was dried under vacuum to afford 2-((6-((2,5-dichloropyrimidin-4-yl)amino)-1-isopropyl-2-oxo-1,2-dihydro-1,8-naphthyridin-3-yl)oxy)-N-methylacetamide (592 mg) as a gray solid.

LC-MS (ESI): m/z=437.1 [M+H]⁺.

### Preparation of 2-((6-((5-chloro-2-(4-((1-(3-(2,6-dioxopiperidin-3-yl)-1-methyl-1H-indazol-7-yl)piperidin-4-yl)methyl) piperazin-1-yl)pyrimidin-4-yl)amino)-1-isopropyl-2-oxo-1,2-dihydro-1,8-naphthyridin-3-yl)oxy)-N-methylacetamide

To a stirred solution of 2-((6-((2,5-dichloropyrimidin-4-yl)amino)-1-isopropyl-2-oxo-1,2-dihydro-1,8-naphthyridin-3-yl)oxy)-N-methylacetamide (0.15 g, 0.34 mmol) and 3-(1-methyl-7-(4-(piperazin-1-ylmethyl)piperidin-1-yl)-1H-indazol-3-yl)piperidine-2,6-dione (0.21 g, 0.52 mmol) in DMSO (3.0 mL) was added N, N-diisopropylethylamine (0.4 mL, 2.29 mmol). The reaction mixture was stirred at 100° C. for 5 hours. The reaction mixture was poured into ice-cold water and stirred for 15 minutes. The resulting precipitate was filtered and dried to afford crude product. The crude product was purified by prep-HPLC to afford 2-((6-((5-chloro-2-(4-((1-(3-(2,6-dioxopiperidin-3-yl)-1-methyl-1H-indazol-7-yl)piperidin-4-yl)methyl) piperazin-1-yl)pyrimidin-4-yl)amino)-1-isopropyl-2-oxo-1,2-dihydro-1,8-naphthyridin-3-yl)oxy)-N-methylacetamide (69 mg) as an off-white solid.

LC-MS (ESI): m/z=825.57 [M+H]⁺.

¹H NMR (400 MHz, DMSO-d₆): δ 10.87 (s, 1H), 9.08 (s, 1H), 8.66 (d, J=2.40 Hz, 1H), 8.09 (s, 1H), 7.95 (d, J=4.40 Hz, 1H), 7.37-7.35 (dd, J=2.40, 6.40 Hz, 1H), 7.11 (s, 1H), 7.01-6.99 (m, 2H), 5.96 (br s, 1H), 4.59 (s, 2H), 4.32-4.31 (m, 1H), 4.24 (s, 3H), 3.63 (s, 4H), 3.23-3.21 (m, 2H), 2.65-2.64 (m, 7H), 2.41 (bs, 4H), 2.32-2.31 (m, 3H), 2.26-2.24 (m, 2H), 1.90-1.89 (m, 3H), 1.85-1.81 (m, 6H), 1.40 (br s, 2H).

### Example 10. Synthesis of 2-((6-((5-chloro-2-(4-((1-(3-(2,6-dioxopiperidin-3-yl)-1-methyl-1H-indazol-6-yl)piperidin-4-yl)methyl) piperazin-1-yl)pyrimidin-4-yl)amino)-1-isopropyl-2-oxo-1,2-dihydro-1,8-naphthyridin-3-yl)oxy)-N-methylacetamide (Compound 104)

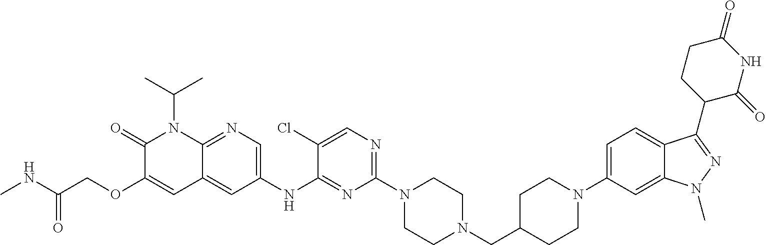 [SMILES: CNC(=O)COc1cc2cc(Nc3nc(N4CCN(CC5CCN(c6ccc7c(C8CCC(=O)NC8=O)nn(C)c7c6)CC5)CC4)ncc3Cl)cnc2n(C(C)C)c1=O]

### Preparation of 2-((6-((5-chloro-2-(4-((1-(3-(2,6-dioxopiperidin-3-yl)-1-methyl-1H-indazol-6-yl)piperidin-4-yl)methyl) piperazin-1-yl)pyrimidin-4-yl)amino)-1-isopropyl-2-oxo-1,2-dihydro-1,8-naphthyridin-3-yl)oxy)-N-methylacetamide

To a stirred solution of 2-((6-((2,5-dichloropyrimidin-4-yl)amino)-1-isopropyl-2-oxo-1,2-dihydro-1,8-naphthyridin-3-yl)oxy)-N-methylacetamide (0.15 g, 0.34 mmol) and 3-(1-methyl-6-(4-(piperazin-1-ylmethyl)piperidin-1-yl)-1H-indazol-3-yl)piperidine-2,6-dione hydrochloride (0.21 g, 0.52 mmol) in DMSO (3.0 mL) was added N,N-diisopropylethylamine (0.4 mL, 2.29 mmol). The reaction mixture was stirred at 100° C. for 5 hours. The reaction mixture was poured into ice-cold water and stirred for 15 minutes. The resulting precipitate was filtered and dried to afford crude product. The crude product was purified by prep-HPLC to afford 2-((6-((5-chloro-2-(4-((1-(3-(2,6-dioxopiperidin-3-yl)-1-methyl-1H-indazol-6-yl)piperidin-4-yl)methyl) piperazin-1-yl)pyrimidin-4-yl)amino)-1-isopropyl-2-oxo-1,2-dihydro-1,8-naphthyridin-3-yl)oxy)-N-methylacetamide (14 mg) as a pale brown solid. LC-MS (ESI): m/z=825.45 [M+H]⁺.

¹H NMR (400 MHz, DMSO-d₆): δ 10.84 (s, 1H), 9.08 (s, 1H), 8.66 (d, J=2.40 Hz, 1H), 8.32 (d, J=2.40 Hz, 1H), 8.09 (s, 1H), 7.94 (d, J=4.40 Hz, 1H), 7.47 (d, J=8.80 Hz, 1H), 7.11 (s, 2H), 6.90 (d, J=8.80 Hz, 1H), 6.82 (s, 1H), 4.58 (s, 2H), 4.24-4.23 (m, 1H), 3.88 (s, 3H), 3.73 (d, J=55.60 Hz, 2H), 2.66 (s, 4H), 2.66-2.64 (m, 7H), 2.39-2.31 (m, 4H), 2.21-2.19 (m, 3H), 1.81-1.76 (m, 3H), 1.56 (d, J=6.80 Hz, 6H), 1.26 (d, J=11.60 Hz, 3H).

### Example 11. Synthesis of 2-((6-((5-chloro-2-((3S)-4-((1-(3-(2,6-dioxopiperidin-3-yl)-1-methyl-1H-indazol-7-yl)piperidin-4-yl)methyl)-3-methylpiperazin-1-yl)pyrimidin-4-yl)amino)-1-isopropyl-2-oxo-1,2-dihydro-1,8-naphthyridin-3-yl)oxy)-N-methylacetamide (Compound 105a)

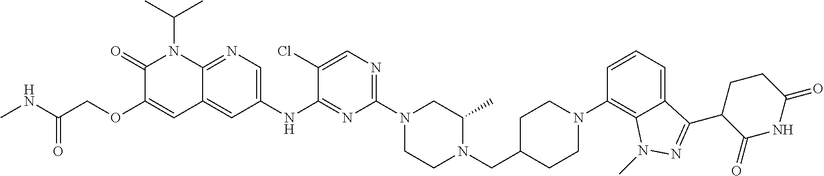 [SMILES: CNC(=O)COc1cc2cc(Nc3nc(N4CCN(CC5CCN(c6cccc7c(C8CCC(=O)NC8=O)nn(C)c67)CC5)[C@@H](C)C4)ncc3Cl)cnc2n(C(C)C)c1=O]

### Preparation of tert-butyl(S)-4-((1-((benzyloxy) carbonyl)piperidin-4-yl)methyl)-3-methylpiperazine-1-carboxylate

To a stirred solution of tert-butyl(S)-3-methylpiperazine-1-carboxylate (10 g, 49.93 mmol) in DCM (200 mL), acetic acid (2.99 mL, 49.9 mmol) and benzyl 4-formylpiperidine-1-carboxylate (6.17 g, 25.0 mmol) were added at 0° C. After stirring for 5 hours, sodium triacetoxyborohydride (26.5 g, 125 mmol) was added. The reaction was allowed to warm to room temperature over 16 hours. The reaction mixture was quenched with ice-cold saturated ammonium chloride solution (200 mL) and extracted into DCM (400 mL×3). The separated organic layers were washed with brine solution (100 mL), dried over anhydrous Na₂SO₄, filtered, and concentrated under reduced pressure to obtain crude product tert-butyl (S)-4-((1-((benzyloxy) carbonyl)piperidin-4-yl)methyl)-3-methylpiperazine-1-carboxylate (8.0 g) as a light yellow semi-solid.

LC-MS (ESI): m/z=432.54 [M+H]⁺.

### Preparation of tert-butyl(S)-3-methyl-4-(piperidin-4-ylmethyl) piperazine-1-carboxylate

To a stirred solution of tert-butyl(S)-4-((1-((benzyloxy) carbonyl)piperidin-4-yl)methyl)-3-methylpiperazine-1-carboxylate (8.0 g, 18.5 mmol) in THF (120 mL) was added 20% Pd (OH)₂/C (4.0 g, 50% w/w). The reaction was stirred under a hydrogen atmosphere (80 psi) at room temperature for 16 hours. The reaction mixture was diluted with DCM (100 mL) and filtered through a celite bed. The celite was washed with 30% THF in DCM (300 mL) and the collected filtrate was concentrated under vacuum to obtain crude product. The crude product was washed with n-pentane (30 mL) and dried under vacuum to obtain tert-butyl(S)-3-methyl-4-(piperidin-4-ylmethyl) piperazine-1-carboxylate (6.0 g) as a pale brown solid. LC-MS (ESI): m/z=297.24 [M+H]⁺.

### Preparation of tert-butyl(S)-4-((1-(3-(2,6-bis(benzyloxy)pyridin-3-yl)-1-methyl-1H-indazol-7-yl)piperidin-4-yl)methyl)-3-methylpiperazine-1-carboxylate

To a stirred solution of 3-(2,6-bis(benzyloxy)pyridin-3-yl)-6-bromo-1-methyl-1H-indazole (1.50 g, 2.99 mmol) in dioxane (7.5 mL) and DMF (1.5 mL) was added tert-butyl(S)-3-methyl-4-(piperidin-4-ylmethyl) piperazine-1-carboxylate (2.67 g, 8.99 mmol) and cesium carbonate (2.93 g, 8.99 mmol). The reaction was degassed with argon for 5 minutes and Pd-PEPPSI-iHeptCl (0.14 g, 0.15 mmol) was added. The reaction was heated at 100° C. for 16 hours. The reaction mixture was diluted with DCM (50 mL) and filtered through a celite bed and washed with THF: DCM (30%) (300 mL). The collected filtrate was concentrated under vacuum to obtain crude product. The crude product was purified by flash chromatography (230-400 silica gel; 30-27% EtOAc in petroleum ether) to afford tert-butyl(S)-4-((1-(3-(2,6-bis(benzyloxy)pyridin-3-yl)-1-methyl-1H-indazol-7-yl)piperidin-4-yl)methyl)-3-methylpiperazine-1-carboxylate (0.80 g) as a pale brown semi-solid.

LC-MS (ESI): m/z=717.79 [M+H]⁺.

### Preparation of tert-butyl (3S)-4-((1-(3-(2,6-dioxopiperidin-3-yl)-1-methyl-1H-indazol-7-yl)piperidin-4-yl)methyl)-3-methylpiperazine-1-carboxylate

To a stirred solution of tert-butyl(S)-4-((1-(3-(2,6-bis(benzyloxy)pyridin-3-yl)-1-methyl-1H-indazol-7-yl)piperidin-4-yl)methyl)-3-methylpiperazine-1-carboxylate (0.71 g, 1.00 mmol) in THF (100 mL) was added 20% Pd (OH)₂/C (0.71 g). The reaction was put under hydrogen atmosphere (80 psi) and stirred at room temperature for 16 hours. The reaction mixture was diluted with DCM (100 mL) and filtered through a celite bed. The celite was washed with 30% THF in DCM (300 mL) and the collected filtrate was concentrated under vacuum to obtain tert-butyl (3S)-4-((1-(3-(2,6-dioxopiperidin-3-yl)-1-methyl-1H-indazol-7-yl)piperidin-4-y1) methyl)-3-methylpiperazine-1-carboxylate (0.65 g) as a light yellow semi-solid. LC-MS (ESI): m/z=539.41 [M+H]⁺.

### Preparation of 3-(1-methyl-7-(4-(((S)-2-methylpiperazin-1-yl)methyl)piperidin-1-yl)-1H-indazol-3-yl)piperidine-2,6-dione

To a stirred solution of tert-butyl (3S)-4-((1-(3-(2,6-dioxopiperidin-3-yl)-1-methyl-1H-indazol-7-yl)piperidin-4-yl)methyl)-3-methylpiperazine-1-carboxylate (0.650 g, 1.20 mmol) in DCM (10 mL) was added trifluoroacetic acid (5.0 mL). The reaction was stirred at room temperature for 2 hours. The reaction mixture was concentrated under vacuum to get afford 3-(1-methyl-7-(4-(((S)-2-methylpiperazin-1-yl)methyl)piperidin-1-yl)-1H-indazol-3-yl)piperidine-2,6-dione (0.60 g) as a pale brown semi-solid.

LC-MS (ESI): m/z=439.57 [M+H]⁺.

### Preparation of 2-((6-((5-chloro-2-((3S)-4-((1-(3-(2,6-dioxopiperidin-3-yl)-1-methyl-1H-indazol-7-yl)piperidin-4-yl)methyl)-3-methylpiperazin-1-yl)pyrimidin-4-yl)amino)-1-isopropyl-2-oxo-1,2-dihydro-1,8-naphthyridin-3-yl)oxy)-N-methylacetamide

To a stirred solution of 3-(1-methyl-7-(4-(((S)-2-methylpiperazin-1-yl)methyl)piperidin-1-yl)-1H-indazol-3-yl)piperidine-2,6-dione (0.20 g, 0.45 mmol) in DMSO (4 mL) was added 2-((6-((2,5-dichloropyrimidin-4-yl)amino)-1-isopropyl-2-oxo-1,2-dihydro-1,8-naphthyridin-3-yl)oxy)-N-methylacetamide (0.099 g, 0.22 mmol) and DIPEA (0.79 mL, 4.5 mmol). The reaction was heated at 100° C. for 12 hours. The solvent was removed using a centrifugal evaporator to obtain crude product. The crude product was purified by prep HPLC to afford 2-((6-((5-chloro-2-((3S)-4-((1-(3-(2,6-dioxopiperidin-3-yl)-1-methyl-1H-indazol-7-yl)piperidin-4-yl)methyl)-3-methylpiperazin-1-yl)pyrimidin-4-yl)amino)-1-isopropyl-2-oxo-1,2-dihydro-1,8-naphthyridin-3-yl)oxy)-N-methylacetamide (43 mg) as an off-white solid. LC-MS (ESI): m/z=837.43 [M−H]⁻.

¹H NMR (400 MHz, DMSO-d₆): δ 10.90 (s, 1H), 9.09 (s, 1H), 8.67 (d, J=2.40 Hz, 1H), 8.30 (d, J=2.00 Hz, 1H), 8.08 (s, 1H), 7.94 (d, J=4.80 Hz, 1H), 7.36-7.35 (m, 1H), 7.12 (s, 1H), 7.10-7.00 (m, 2H), 5.90 (s, 1H), 4.60 (s, 2H), 4.40-4.30 (m, 1H), 4.20 (s, 3H), 4.02-4.00 (d, J=10.0 Hz, 2H), 3.20 (br, 3H), 2.89-2.86 (m, 2H), 2.61-2.59 (m, 8H), 2.45-2.31 (m, 2H), 2.25-1.98 (m, 4H), 1.80-1.60 (m, 2H), 1.58 (d, J=6.80 Hz, 6H), 1.50-1.20 (m, 2H), 1.00 (d, J=Hz, 3H).

### Example 12. Synthesis of 2-((6-((5-chloro-2-((3S)-4-((1-(3-(2,6-dioxopiperidin-3-yl)-1-methyl-1H-indazol-6-yl)piperidin-4-yl)methyl)-3-methylpiperazin-1-yl)pyrimidin-4-yl)amino)-1-isopropyl-2-oxo-1,2-dihydro-1,8-naphthyridin-3-yl)oxy)-N-methylacetamide (Compound 106a)

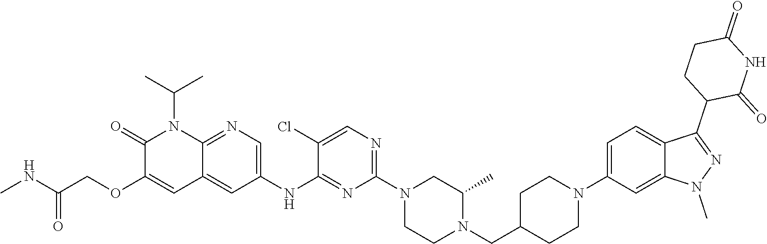 [SMILES: CNC(=O)COc1cc2cc(Nc3nc(N4CCN(CC5CCN(c6ccc7c(C8CCC(=O)NC8=O)nn(C)c7c6)CC5)[C@@H](C)C4)ncc3Cl)cnc2n(C(C)C)c1=O]

### Preparation of tert-butyl(S)-4-((1-(3-(2,6-bis(benzyloxy)pyridin-3-yl)-1-methyl-1H-indazol-6-yl)piperidin-4-yl)methyl)-3-methylpiperazine-1-carboxylate

To stirred solution of 3-(2,6-bis(benzyloxy)pyridin-3-yl)-6-bromo-1-methyl-1H-indazole (1.0 g, 1.99 mmol) in dioxane (12.0 mL), tert-butyl(S)-3-methyl-4-(piperidin-4-ylmethyl) piperazine-1-carboxylate (1.78 g, 5.99 mmol) and NaOtBu (0.58 g, 5.99 mmol) were added. The reaction was degassed with argon for 5 minutes. Brettphos Pd G3 (0.09 g, 0.10 mmol) was added, and the reaction was heated at 100° C. for 18 h. The reaction mixture was quenched with water (5 mL) and extracted with ethyl acetate (50 mL). The separated organic layers were dried over anhydrous Na₂SO₄, filtered, and dried under vacuum to obtain crude product. The crude product was purified by flash column chromatography (230-400 mesh silica gel; 3% Methanol in DCM as eluent) to afford tert-butyl(S)-4-((1-(3-(2,6-bis(benzyloxy)pyridin-3-yl)-1-methyl-1H-indazol-6-yl)piperidin-4-yl)methyl)-3-methylpiperazine-1-carboxylate (0.96 g) as a brown semi-solid.

LC-MS (ESI): m/z=717.83 [M+H]⁺.

### Preparation of tert-butyl (3S)-4-((1-(3-(2,6-dioxopiperidin-3-yl)-1-methyl-1H-indazol-6-yl)piperidin-4-yl)methyl)-3-methylpiperazine-1-carboxylate

To a stirred solution of tert-butyl(S)-4-((1-(3-(2,6-bis(benzyloxy)pyridin-3-yl)-1-methyl-1H-indazol-6-yl)piperidin-4-yl)methyl)-3-methylpiperazine-1-carboxylate (0.96 g, 1.35 mmol) in THF (38 mL), 20% Pd (OH)₂/C (0.96 g, 1.35 mmol) was added. The reaction was put under a hydrogen atmosphere (80 psi) and stirred at room temperature for 16 h. The reaction mixture was diluted with DCM (10 mL) and filtered through a celite bed. The celite was washed with 20% THF: DCM (200 mL). The collected filtrate was concentrated under vacuum to afford tert-butyl (3S)-4-((1-(3-(2,6-dioxopiperidin-3-yl)-1-methyl-1H-indazol-6-yl)piperidin-4-yl)methyl)-3-methylpiperazine-1-carboxylate (0.96 g) as a pale brown semi-solid.

LC-MS (ESI): m/z=539.83 [M+H]⁺.

### Preparation of 3-(1-methyl-6-(4-(((S)-2-methylpiperazin-1-yl)methyl)piperidin-1-yl)-1H-indazol-3-yl)piperidine-2,6-dione

To a stirred solution of tert-butyl (3S)-4-((1-(3-(2,6-dioxopiperidin-3-yl)-1-methyl-1H-indazol-6-yl)piperidin-4-yl)methyl)-3-methylpiperazine-1-carboxylate (0.80 g, 1.49 mmol) in DCM (8.0 mL), TFA (8.0 mL) was added at 0° C. The reaction was allowed to warm to room temperature with stirring over 3 h. The reaction mixture was concentrated under vacuum to obtain crude product. The crude product was triturated with petroleum ether (3×10 mL) and dried to afford 3-(1-methyl-6-(4-(((S)-2-methylpiperazin-1-yl)methyl)piperidin-1-yl)-1H-indazol-3-yl)piperidine-2,6-dione (TFA salt) (0.80 g) as a pale brown semi-solid.

LC-MS (ESI): m/z=439.83 [M+H]⁺.

### Preparation of 2-((6-((5-chloro-2-((3S)-4-((1-(3-(2,6-dioxopiperidin-3-yl)-1-methyl-1H-indazol-6-yl)piperidin-4-yl)methyl)-3-methylpiperazin-1-yl)pyrimidin-4-yl)amino)-1-isopropyl-2-oxo-1,2-dihydro-1,8-naphthyridin-3-yl)oxy)-N-methylacetamide

To a stirred solution of 3-(1-methyl-6-(4-(((S)-2-methylpiperazin-1-yl)methyl)piperidin-1-yl)-1H-indazol-3-yl)piperidine-2,6-dione (TFA salt) (0.20 g, 0.45 mmol) in DMSO (4 mL) was added 2-((6-((2,5-dichloropyrimidin-4-yl)amino)-1-isopropyl-2-oxo-1,2-dihydro-1,8-naphthyridin-3-yl)oxy)-N-methylacetamide (0.099 g, 0.22 mmol) and DIPEA (0.79 mL, 4.5 mmol). The reaction was heated at 100° C. for 12 hours. Solvent was removed using a centrifugal evaporator to obtain crude product. The crude product was purified by prep HPLC to afford 2-((6-((5-chloro-2-((3S)-4-((1-(3-(2,6-dioxopiperidin-3-yl)-1-methyl-1H-indazol-6-yl)piperidin-4-yl)methyl)-3-methylpiperazin-1-yl)pyrimidin-4-yl)amino)-1-isopropyl-2-oxo-1,2-dihydro-1,8-naphthyridin-3-yl)oxy)-N-methylacetamide (39 mg) as an off-white solid. ¹H NMR (400 MHz, DMSO-d₆): δ 10.84 (s, 1H), 9.08 (s, 1H), 8.67 (d, J=2.40 Hz, 1H), 8.29 (d, J=2.00 Hz, 1H), 8.08 (s, 1H), 7.94 (d, J=4.40 Hz, 1H), 7.47 (d, J=9.20 Hz, 1H), 7.12 (s, 1H), 6.90 (d, J=8.80 Hz, 1H), 6.82 (s, 1H), 5.90 (s, 1H), 4.56 (s, 1H), 4.24-4.23 (m, 1H), 4.01-3.98 (m, 2H), 3.88 (s, 3H), 3.77 (d, J=12.00 Hz, 2H), 3.32-3.23 (br, 1H), 3.00-2.80 (m, 2H), 2.80-2.60 (m, 8H), 2.59 (br, 1H), 2.42-2.31 (m, 2H), 2.34-2.12 (m, 2H), 2.01-1.91 (m, 2H), 1.75-1.72 (m, 2H), 1.58 (d, J=6.80 Hz, 6H), 1.23-1.20 (m, 2H), 0.97 (d, J=6.00 Hz, 3H). LC-MS (ESI): m/z=839.55 [M+H]⁺.

### Example 13. Synthesis of 2-((6-((5-chloro-2-(7-((3-(2,6-dioxopiperidin-3-yl)-1-methyl-1H-indazol-7-yl)amino)-2-azaspiro[3.5]nonan-2-yl)pyrimidin-4-yl)amino)-1-methyl-2-oxo-1,2-dihydro-1,8-naphthyridin-3-yl)oxy)-N-methylacetamide (Compound 137)

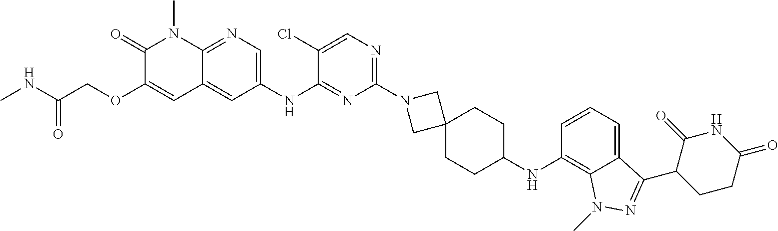 [SMILES: CNC(=O)COc1cc2cc(Nc3nc(N4CC5(CCC(Nc6cccc7c(C8CCC(=O)NC8=O)nn(C)c67)CC5)C4)ncc3Cl)cnc2n(C)c1=O]

### Preparation of tert-butyl 7-((3-(2,6-bis(benzyloxy)pyridin-3-yl)-1-methyl-1H-indazol-7-yl)amino)-2-azaspiro[3.5]nonane-2-carboxylate

A stirred solution of 3-(2,6-bis(benzyloxy)pyridin-3-yl)-7-bromo-1-methyl-1H-indazole (0.60 g, 1.20 mmol), tert-butyl 7-amino-2-azaspiro[3.5]nonane-2-carboxylate (0.86 g, 3.60 mmol), and cesium carbonate (1.17 g, 3.60 mmol) in dioxane (6 mL) was degassed with argon for 5 minutes. Pd-PEPPSI-iHeptC1 (0.06 g, 0.06 mmol) was added, the reaction flushed for 5 minutes with argon, and the reaction was heated to 100° C. for 18 hours. The reaction mixture was filtered through a celite bed and washed with excess of EtOAc (200 mL). The filtrate was concentrated under vacuum to obtain crude product. The crude product was purified by flash chromatography (230-400 mesh silica gel, 50%-52% of ethyl acetate in petroleum ether as eluent) to afford tert-butyl 7-((3-(2,6-bis(benzyloxy)pyridin-3-yl)-1-methyl-1H-indazol-7-yl)amino)-2-azaspiro[3.5]nonane-2-carboxylate (0.50 g) as a pale yellow semi-solid. LC-MS (ESI): m/z=660.46 [M+H]⁺.

### Preparation of tert-butyl 7-((3-(2,6-dioxopiperidin-3-yl)-1-methyl-1H-indazol-7-yl)amino)-2-azaspiro[3.5]nonane-2-carboxylate

To a stirred solution of tert-butyl 7-((3-(2,6-bis(benzyloxy)pyridin-3-yl)-1-methyl-1H-indazol-7-yl)amino)-2-azaspiro[3.5]nonane-2-carboxylate (0.50 g, 0.76 mmol) in THF (65 mL), 20% Pd (OH)₂/C (0.75 g) was added. The reaction was put under H₂ atmosphere (80 psi) and stirred at room temperature for 16 hours. The reaction mixture was diluted with DCM (100 mL) and filtered through a celite bed. The collected filtrate was concentrated under vacuum to afford tert-butyl 7-((3-(2,6-dioxopiperidin-3-yl)-1-methyl-1H-indazol-7-yl)amino)-2-azaspiro[3.5]nonane-2-carboxylate (0.40 g) as a pale brown semi-solid.

LC-MS (ESI): m/z=482.59 [M+H]⁺.

### Preparation of 3-(7-((2-azaspiro[3.5]nonan-7-yl)amino)-1-methyl-1H-indazol-3-yl)piperidine-2,6-dione

To a stirred solution of tert-butyl 7-((3-(2,6-dioxopiperidin-3-yl)-1-methyl-1H-indazol-7-yl)amino)-2-azaspiro[3.5]nonane-2-carboxylate (0.40 g, 0.83 mmol) in DCM (4 mL) was added trifluoroacetic acid (3.2 mL) at 0° C. The reaction was allowed to warm to room temperature over 2 hours with stirring. The reaction mixture was concentrated under reduced pressure to afford 3-(7-((2-azaspiro[3.5]nonan-7-yl)amino)-1-methyl-1H-indazol-3-yl)piperidine-2,6-dione (0.35 g) as a pale brown semi-solid.

LC-MS (ESI): m/z=382.48 [M−H]⁻.

### Preparation of 2-((6-((5-chloro-2-(7-((3-(2,6-dioxopiperidin-3-yl)-1-methyl-1H-indazol-7-yl)amino)-2-azaspiro[3.5]nonan-2-yl)pyrimidin-4-yl)amino)-1-methyl-2-oxo-1,2-dihydro-1,8-naphthyridin-3-yl)oxy)-N-methylacetamide

To a stirred solution of 3-(7-((2-azaspiro[3.5]nonan-7-yl)amino)-1-methyl-1H-indazol-3-yl)piperidine-2,6-dione (0.35 g, 0.92 mmol) and 2-((6-((2,5-dichloropyrimidin-4-yl)amino)-1-methyl-2-oxo-1,2-dihydro-1,8-naphthyridin-3-yl)oxy)-N-methylacetamide (0.17 g, 0.41 mmol) in DMSO (3.01 mL), DIPEA (3.01 mL) was added. The reaction was at 100° C. for 8 hours. Solvent was removed using a centrifugal evaporator to obtain crude product. The crude product was purified by prep-HPLC to afford 2-((6-((5-chloro-2-(7-((3-(2,6-dioxopiperidin-3-yl)-1-methyl-1H-indazol-7-yl)amino)-2-azaspiro[3.5]nonan-2-yl)pyrimidin-4-yl)amino)-1-methyl-2-oxo-1,2-dihydro-1,8-naphthyridin-3-yl)oxy)-N-methylacetamide (0.13 g) as an off-white solid.

LC-MS (ESI): m/z=754.58 [M+H]⁺.

¹H NMR: (400 MHz, DMSO-d₆): δ 10.85 (s, 1H), 9.03 (s, 1H), 8.83 (d, J=2.40 Hz, 1H), 8.25 (s, 1H), 8.07 (s, 1H), 7.96 (d, J=4.40 Hz, 1H), 7.19 (s, 1H), 6.97 (d, J=8.00 Hz, 1H), 6.87-6.89 (m, 1H), 6.54 (d, J=7.20 Hz, 1H), 4.94 (d, J=5.60 Hz, 1H), 4.64 (s, 2H), 4.26-4.25 (m, 1H), 4.22 (s, 3H), 3.74 (s, 3H), 3.68 (s, 4H), 2.65 (br, 1H), 2.61-2.60 (m, 5H), 2.27-2.26 (m, 1H), 2.13-2.12 (m, 1H), 1.93-1.91 (m, 4H), 1.67-1.66 (m, 2H), 1.52-1.49 (m, 2H).

### Example 14. Synthesis of 2-((6-((5-chloro-2-(7-((3-(2,6-dioxopiperidin-3-yl)-1-methyl-1H-indazol-6-yl)amino)-2-azaspiro[3.5]nonan-2-yl)pyrimidin-4-yl)amino)-1-methyl-2-oxo-1,2-dihydro-1,8-naphthyridin-3-yl)oxy)-N-methylacetamide (Compound 138)

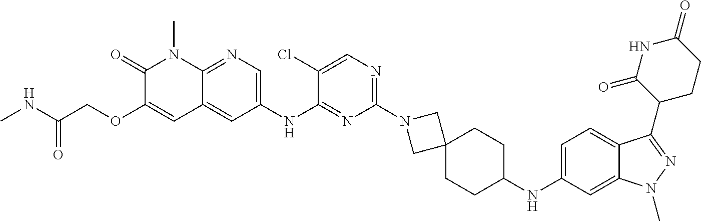 [SMILES: CNC(=O)COc1cc2cc(Nc3nc(N4CC5(CCC(Nc6ccc7c(C8CCC(=O)NC8=O)nn(C)c7c6)CC5)C4)ncc3Cl)cnc2n(C)c1=O]

### Preparation of tert-butyl 7-((3-(2,6-bis(benzyloxy)pyridin-3-yl)-1-methyl-1H-indazol-6-yl)amino)-2-azaspiro[3.5]nonane-2-carboxylate

A stirred solution of 3-(2,6-bis(benzyloxy)pyridin-3-yl)-6-bromo-1-methyl-1H-indazole (0.80 g, 1.60 mmol), tert-butyl 7-amino-2-azaspiro[3.5]nonane-2-carboxylate (0.58 g, 2.40 mmol), KOtBu (0.54 g, 4.80 mmol) in 1,4-dioxane (20 mL) was degassed with argon for 10 minutes. Brettphos Pd G3 (0.14 g, 0.16 mmol) was added, the reaction was purged with argon for 5 minutes and heated to 100° C. for 2 hours. The reaction mixture was filtered through a celite bed and washed with ethyl acetate (100 mL). Th collected filtrate was concentrated under vacuum to obtain crude product. The crude product was purified by flash column chromatography (SiO₂, 230-400, 40% ethyl acetate in petroleum ether as eluent) to afford tert-butyl 7-((3-(2,6-bis(benzyloxy)pyridin-3-yl)-1-methyl-1H-indazol-6-yl)amino)-2-azaspiro[3.5]nonane-2-carboxylate (0.90 g) as an off-white solid.

LC-MS (ESI): m/z=661.66 [M+2]⁺.

### Preparation of tert-butyl 7-((3-(2,6-dioxopiperidin-3-yl)-1-methyl-1H-indazol-6-yl)amino)-2-azaspiro[3.5]nonane-2-carboxylate

To a stirred solution of tert-butyl 7-((3-(2,6-bis(benzyloxy)pyridin-3-yl)-1-methyl-1H-indazol-6-yl)amino)-2-azaspiro[3.5]nonane-2-carboxylate (0.90 g, 1.36 mmol) in THF (27.0 mL) was added 20% Pd (OH)₂ (0.90 g, 100% w/w). The reaction was put under H₂ (70 psi) atmosphere and stirred at room temperature for 16 hours. The reaction mixture was filtered through a celite bed and washed with ethyl acetate (200 mL). The collected filtrate was concentrated under reduced pressure to afford tert-butyl 7-((3-(2,6-dioxopiperidin-3-yl)-1-methyl-1H-indazol-6-yl)amino)-2-azaspiro[3.5]nonane-2-carboxylate (0.70 g) as a brown solid.

LC-MS (ESI): m/z=482.57 [M+H]⁺.

### Preparation of 3-(6-((2-azaspiro[3.5]nonan-7-yl)amino)-1-methyl-1H-indazol-3-yl)piperidine-2,6-dione

To a stirred solution of tert-butyl 7-((3-(2,6-dioxopiperidin-3-yl)-1-methyl-1H-indazol-6-yl)amino)-2-azaspiro[3.5]nonane-2-carboxylate (0.70 g, 1.45 mmol) in DCM (14 mL) cooled to 0° C., TFA (7 mL) was added. The reaction was allowed to warm to room temperature over 3 hours with stirring. The reaction mixture was concentrated to obtain crude product. The crude product was triturated with diethyl ether and dried under vacuum to afford 3-(6-((2-azaspiro[3.5]nonan-7-yl)amino)-1-methyl-1H-indazol-3-yl)piperidine-2,6-dione (0.90 g) as an off-white solid.

LC-MS (ESI): m/z=382.41 [M+H]⁺.

### Preparation of 2-((6-((5-chloro-2-(7-((3-(2,6-dioxopiperidin-3-yl)-1-methyl-1H-indazol-6-yl)amino)-2-azaspiro[3.5]nonan-2-yl)pyrimidin-4-yl)amino)-1-methyl-2-oxo-1,2-dihydro-1,8-naphthyridin-3-yl)oxy)-N-methylacetamide

To a stirred solution of 3-(6-((2-azaspiro[3.5]nonan-7-yl)amino)-1-methyl-1H-indazol-3-yl)piperidine-2,6-dione (0.30 g, 0.78 mmol) and 2-((6-((2,5-dichloropyrimidin-4-yl)amino)-1-methyl-2-oxo-1,2-dihydro-1,8-naphthyridin-3-yl)oxy)-N-methylacetamide (0.12 g, 0.31 mmol) in DMSO (3 mL), DIPEA (2.05 mL, 11.8 mmol) was added. The reaction was heated to 100° C. for 8 hours. Solvent was removed using a centrifugal evaporator to afford crude product. The crude product was purified via prep-HPLC to afford 2-((6-((5-chloro-2-(7-((3-(2,6-dioxopiperidin-3-yl)-1-methyl-1H-indazol-6-yl)amino)-2-azaspiro[3.5]nonan-2-yl)pyrimidin-4-yl)amino)-1-methyl-2-oxo-1,2-dihydro-1,8-naphthyridin-3-yl)oxy)-N-methylacetamide (0.08 g) as an off-white solid.

LC-MS (ESI): m/z=752.36 [M−H]⁻.

¹H NMR data (400 MHz, DMSO-d₆): δ 10.88 (s, 1H), 9.04 (s, 1H), 8.84 (d, J=2.40 Hz, 1H), 8.52-8.46 (m, 1H), 8.07 (s, 1H), 7.97 (d, J=4.80 Hz, 1H), 7.31 (d, J=8.80 Hz, 1H), 7.21 (s, 1H), 6.51 (d, J=8.80 Hz, 1H), 6.38 (s, 1H), 5.71 (d, J=8.00 Hz, 1H), 4.64 (s, 2H), 4.17-4.15 (m, 1H), 3.81 (s, 3H), 3.74 (d, J=3.60 Hz, 4H), 3.68 (s, 3H), 3.38-3.32 (m, 1H), 2.67 (d, J=4.40 Hz, 3H), 2.60-2.58 (m, 2H), 2.22-2.21 (m, 1H), 2.13-2.12 (m, 1H), 1.93 (d, J=10.40 Hz, 4H), 1.65-1.62 (m, 2H), 1.29-1.26 (m, 2H).

### Example 15. Synthesis of 2-((6-((5-chloro-2-(6-((1-(3-(2,6-dioxopiperidin-3-yl)-1-methyl-1H-indazol-6-yl)piperidin-4-yl)methyl)-2,6-diazaspiro[3.3]heptan-2-yl)pyrimidin-4-yl)amino)-1-methyl-2-oxo-1,2-dihydro-1,8-naphthyridin-3-yl)oxy)-N-methylacetamide (Compound 118)

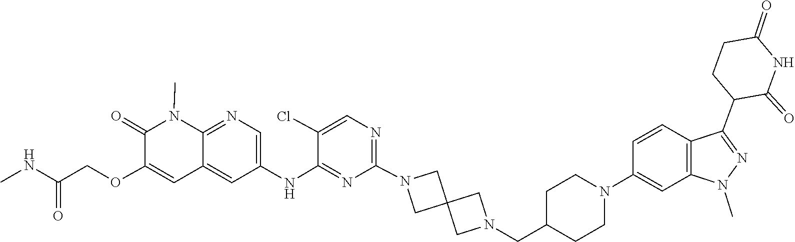 [SMILES: CNC(=O)COc1cc2cc(Nc3nc(N4CC5(CN(CC6CCN(c7ccc8c(C9CCC(=O)NC9=O)nn(C)c8c7)CC6)C5)C4)ncc3Cl)cnc2n(C)c1=O]

### Preparation of tert-butyl 6-((1-((benzyloxy) carbonyl)piperidin-4-yl)methyl)-2,6-diazaspiro[3.3]heptane-2-carboxylate

Tert-butyl 2,6-diazaspiro[3.3]heptane-2-carboxylate (1 g, 5.04 mmol) and benzyl 4-formylpiperidine-1-carboxylate (1.49 g, 6.05 mmol) were dissolved in a mixture of THF:MeOH (1:1) (20 mL) and stirred at room temperature for 1 hour under N₂ atmosphere. After 1 hour, sodium cyanoborohydride (0.63 g, 10.0 mmol) was added at 0° C. The reaction was allowed to warm to room temperature over 16 hours with stirring. The reaction mixture was concentrated under reduced pressure. The crude reaction was diluted with water (100 mL) and extracted by ethyl acetate (3×100 mL). The combined organic layers and concentrated under reduced pressure to afford crude product. The crude product was purified by flash column chromatography (SiO₂, 60-120 mesh, 25% ethyl acetate in petroleum ether) to afford tert-butyl 6-((1-((benzyloxy) carbonyl)piperidin-4-yl)methyl)-2,6-diazaspiro[3.3]heptane-2-carboxylate (0.9 g).

LC-MS (ESI): m/z=430.56 [M+H]⁺.

### Preparation of tert-butyl 6-(piperidin-4-ylmethyl)-2,6-diazaspiro[3.3]heptane-2-carboxylate

A stirred solution of tert-butyl 6-((1-((benzyloxy) carbonyl)piperidin-4-yl)methyl)-2,6-diazaspiro[3.3]heptane-2-carboxylate (0.8 g, 1.86 mmol) in ethanol (16 mL) was purged with N₂ for 5 minutes. Palladium on activated carbon (10%) (0.80 g, 1.86 mmol) was added at room temperature. The reaction mixture was put under H₂ atmosphere (100 psi) and stirred at room temperature for 16 hours. The reaction mixture was filtered through a celite bed, and the celite was washed with ethyl acetate (100 mL). The filtrate was concentrated under reduced pressure to afford tert-butyl 6-(piperidin-4-ylmethyl)-2,6-diazaspiro[3.3]heptane-2-carboxylate (0.50 g).

LC-MS (ESI): m/z=296.21 [M+H]⁺.

### Preparation of tert-butyl 6-((1-(3-(2,6-bis(benzyloxy)pyridin-3-yl)-1-methyl-1H-indazol-6-yl)piperidin-4-yl)methyl)-2,6-diazaspiro[3.3]heptane-2-carboxylate

To a stirred solution of tert-butyl 6-(piperidin-4-ylmethyl)-2,6-diazaspiro[3.3]heptane-2-carboxylate (0.5 g, 1.69 mmol) and 3-(2,6-bis(benzyloxy)pyridin-3-yl)-6-bromo-1-methyl-1H-indazole (0.42 g, 0.84 mmol) in 1,4 dioxane (5 mL), sodium tert-butoxide (0.49 g, 5.07 mmol) was added. The reaction was purged with N₂ for 5 minutes, then Ruphos Pd G3 (0.14 g, 0.16 mmol) was added, and the reaction was sparged with N₂ for 5 minutes and then heated at 100° C. for 16 hours. The reaction mixture was quenched with ice-cold water (50 mL) and extracted with ethyl acetate (2×100 mL). The separated organic layers were washed with brine solution (50 mL), dried over anhydrous sodium sulfate, filtered, and concentrated to afford crude product. The crude product was purified by flash column chromatography (SiO₂, 60-120 mesh, 50-60% ethyl acetate in petroleum ether) to afford tert-butyl 6-((1-(3-(2,6-bis(benzyloxy)pyridin-3-yl)-1-methyl-1H-indazol-6-yl)piperidin-4-yl)methyl)-2,6-diazaspiro[3.3]heptane-2-carboxylate (0.75 g).

LC-MS (ESI): m/z=715.77 [M+H]⁺.

### Preparation of tert-butyl 6-((1-(3-(2,6-dioxopiperidin-3-yl)-1-methyl-1H-indazol-6-yl)piperidin-4-yl)methyl)-2,6-diazaspiro[3.3]heptane-2-carboxylate

A stirred solution of tert-butyl 6-((1-(3-(2,6-bis(benzyloxy)pyridin-3-yl)-1-methyl-1H-indazol-6-yl)piperidin-4-yl)methyl)-2,6-diazaspiro[3.3]heptane-2-carboxylate (0.6 g, 0.83 mmol) and AcOH (0.06 mL) in THF (18 mL) was degassed with nitrogen for 5 minutes and then 20% palladium hydroxide on carbon, 50% w/w (0.60 g) was added to the reaction. The reaction mixture was put under H₂ atmosphere (15 psi) and stirred at room temperature for 16 hours. The reaction mixture was diluted with ethyl acetate (50 mL) and filtered through a celite bed. The filtrate was concentrated to obtain crude product. The crude product was purified by trituration with diethyl ether (2×20 mL) to afford tert-butyl 6-((1-(3-(2,6-dioxopiperidin-3-yl)-1-methyl-1H-indazol-6-yl)piperidin-4-yl)methyl)-2,6-diazaspiro[3.3]heptane-2-carboxylate (0.41 g).

LC-MS (ESI): m/z=537.66 [M+H]⁺.

### Preparation of 3-(6-(4-((2,6-diazaspiro[3.3]heptan-2-yl)methyl)piperidin-1-yl)-1-methyl-1H-indazol-3-yl)piperidine-2,6-dione

To a stirred solution of tert-butyl 6-((1-(3-(2,6-dioxopiperidin-3-yl)-1-methyl-1H-indazol-6-yl)piperidin-4-yl)methyl)-2,6-diazaspiro[3.3]heptane-2-carboxylate (0.4 g, 0.74 mmol) in DCM (4 mL), 4 M HCl in dioxane (2 mL) was added at 0° C. under N₂ atmosphere. The reaction mixture was stirred at room temperature for 3 hours. The reaction mixture was concentrated under reduced pressure to obtain crude product. The crude product was purified by trituration with diethyl ether (2×20 mL) to afford 3-(6-(4-((2,6-diazaspiro[3.3]heptan-2-yl)methyl)piperidin-1-yl)-1-methyl-1H-indazol-3-yl)piperidine-2,6-dione (0.30 g). LC-MS (ESI): m/z=437.59 [M+H]⁺.

### Preparation of 2-((6-((5-chloro-2-(6-((1-(3-(2,6-dioxopiperidin-3-yl)-1-methyl-1H-indazol-6-yl)piperidin-4-yl)methyl)-2,6-diazaspiro[3.3]heptan-2-yl)pyrimidin-4-yl)amino)-1-methyl-2-oxo-1,2-dihydro-1,8-naphthyridin-3-yl)oxy)-N-methylacetamide

To a stirred solution of 3-(6-(4-((2,6-diazaspiro[3.3]heptan-2-yl)methyl)piperidin-1-yl)-1-methyl-1H-indazol-3-yl)piperidine-2,6-dione hydrochloride (0.150 g, 0.31 mmol) and 2-((6-((2,5-dichloropyrimidin-4-yl)amino)-1-methyl-2-oxo-1,2-dihydro-1,8-naphthyridin-3-yl)oxy)-N-methylacetamide (0.06 g, 0.15 mmol) in DMSO, N,N-diisopropylethylamine (0.17 mL, 0.95 mmol) was added at room temperature. The reaction was heated at 100° C. for 3 hours. Solvent was removed using a centrifugal evaporator to obtain crude product. The crude product was purified by prep-HPLC to afford 2-((6-((5-chloro-2-(6-((1-(3-(2,6-dioxopiperidin-3-yl)-1-methyl-1H-indazol-6-yl)piperidin-4-yl)methyl)-2,6-diazaspiro[3.3]heptan-2-yl)pyrimidin-4-yl)amino)-1-methyl-2-oxo-1,2-dihydro-1,8-naphthyridin-3-yl)oxy)-N-methylacetamide (35 mg) as an off-white solid.

LC-MS (ESI): m/z=807.47 [M−H]⁻.

¹H NMR (400 MHz, DMSO-d₆): δ 10.83 (s, 1H), 9.06 (s, 1H), 8.81 (d, J=2.40 Hz, 1H), 8.26 (br s, 1H), 8.39 (d, J=2.40 Hz, 1H), 8.06 (s, 1H), 7.97 (d, J=4.80 Hz, 1H), 7.46 (d, J=8.80 Hz, 1H), 7.24 (s, 1H), 6.88 (dd, J=1.60, 9.20 Hz, 1H), 6.81 (s, 1H), 4.63 (s, 2H), 4.24 (q, J=5.20 Hz, 1H), 4.03 (s, 4H), 3.88 (s, 3H), 3.74-3.73 (m, 5H), 3.28 (br, 4H), 2.65-2.64 (m, 6H), 2.62-2.60 (m, 1H), 2.28-2.26 (m, 3H), 2.14-2.13 (m, 1H), δ 1.75 (d, J=11.20 Hz, 2H), 1.42 (br, 1H)_{1.25}-1.23 (m, 2H).

### Example 16. Synthesis of 2-((6-((5-chloro-2-(9-((1-(3-(2,6-dioxopiperidin-3-yl)-1-methyl-1H-indazol-7-yl)piperidin-4-yl)methyl)-3,9-diazaspiro[5.5]undecan-3-yl)pyrimidin-4-yl)amino)-1-methyl-2-oxo-1,2-dihydro-1,8-naphthyridin-3-yl)oxy)-N-methylacetamide

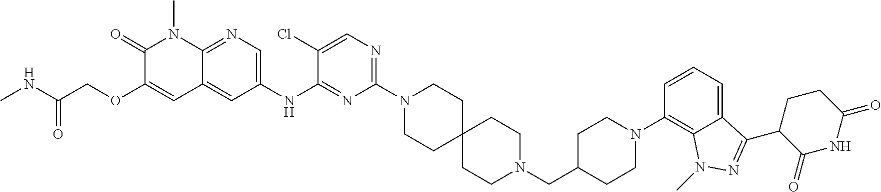 [SMILES: CNC(=O)COc1cc2cc(Nc3nc(N4CCC5(CCN(CC6CCN(c7cccc8c(C9CCC(=O)NC9=O)nn(C)c78)CC6)CC5)CC4)ncc3Cl)cnc2n(C)c1=O]

### Preparation of (1-(3-(2,6-bis(benzyloxy)pyridin-3-yl)-1-methyl-1H-indazol-7-yl)piperidin-4-yl)methanol

To a stirred suspension of 3-(2,6-bis(benzyloxy)pyridin-3-yl)-7-bromo-1-methyl-1H-indazole (2.0 g, 4.0 mmol) in 1,4-dioxane (40 mL) was added piperidin-4-ylmethanol (0.69 g, 6.00 mmol) and cesium carbonate (3.91 g, 12.0 mmol). The reaction was purged with argon for 15 minutes, then PEPPSI iHept C₁ (0.19 g, 0.20 mmol) was added, and the reaction was heated at 100° C. for 5 hours. The reaction mixture was quenched with ice-cold water (30 mL) and extracted into ethyl acetate (3×150 mL). The combined organic layers were washed with brine solution, dried over anhydrous Na₂SO₄, filtered, and concentrated to afford crude product. The crude product was purified by column chromatography (SiO₂, 100-200 mesh, 55% EtOAc in petroleum ether) to afford (1-(3-(2,6-bis(benzyloxy)pyridin-3-yl)-1-methyl-1H-indazol-7-yl)piperidin-4-yl)methanol (0.80 g) as a yellow solid.

LC-MS (ESI): m/z=535.62 [M+H]⁺.

### Preparation of (1-(3-(2,6-bis(benzyloxy)pyridin-3-yl)-1-methyl-1H-indazol-7-yl)piperidin-4-yl)methyl methanesulfonate

A stirred solution of (1-(3-(2,6-bis(benzyloxy)pyridin-3-yl)-1-methyl-1H-indazol-7-yl)piperidin-4-yl)methanol (0.80 g, 1.49 mmol) in DCM (24 mL) was cooled to 0° C. TEA (0.61 mL, 4.49 mmol) and MsCl (0.30 mL, 4.49 mmol) were added, and the reaction mixture was stirred for 2 hours. The reaction mixture was quenched with ice-cold water (30 mL) and extracted into ethyl acetate (3×150 mL). The combined organic layers were washed with brine solution, dried over anhydrous Na₂SO₄, filtered, and concentrated under vacuum to afford crude product. The crude product was washed with n-pentane to afford (1-(3-(2,6-bis(benzyloxy)pyridin-3-yl)-1-methyl-1H-indazol-7-yl)piperidin-4-yl) methyl methanesulfonate (0.8 g) as an orange semi-solid.

LC-MS (ESI): m/z=613.62 [M+H]⁺.

### Preparation of tert-butyl 9-((1-(3-(2,6-bis(benzyloxy)pyridin-3-yl)-1-methyl-1H-indazol-7-yl)piperidin-4-yl)methyl)-3,9-diazaspiro[5.5]undecane-3-carboxylate

To a stirred solution of (1-(3-(2,6-bis(benzyloxy)pyridin-3-yl)-1-methyl-1H-indazol-7-yl)piperidin-4-yl)methyl methanesulfonate (0.8 g, 1.30 mmol) and tert-butyl 3,9-diazaspiro[5.5]undecane-3-carboxylate (1.32 g, 5.22 mmol) in DMSO (16 mL), N,N-diisopropylethylamine (2.27 mL, 13.0 mmol) was added. The reaction mixture was stirred at 100° C. for 16 hours. The reaction mixture was diluted with ice water (50 mL) and extracted with 5% MeOH in DCM (3×70 mL). The organic layers were washed with brine solution, dried over anhydrous Na₂SO₄, filtered, and concentrated under vacuum to afford crude product. The crude product was purified by column chromatography (SiO₂, 100-200 mesh; 52% EtOAc in petroleum ether) to afford tert-butyl 9-((1-(3-(2,6-bis(benzyloxy)pyridin-3-yl)-1-methyl-1H-indazol-7-yl)piperidin-4-yl)methyl)-3,9-diazaspiro[5.5]undecane-3-carboxylate (0.5 g) as an off-white solid.

LC-MS (ESI): m/z=771.79 [M+H]⁺.

### Preparation of tert-butyl 9-((1-(3-(2,6-dioxopiperidin-3-yl)-1-methyl-1H-indazol-7-yl)piperidin-4-yl)methyl)-3,9-diazaspiro[5.5]undecane-3-carboxylate

A stirred solution of tert-butyl 9-((1-(3-(2,6-bis(benzyloxy)pyridin-3-yl)-1-methyl-1H-indazol-7-yl)piperidin-4-yl)methyl)-3,9-diazaspiro[5.5]undecane-3-carboxylate (0.5 g, 0.64 mmol) in THF (15 mL) was degassed with nitrogen. 20% palladium hydroxide (0.5 g) was added to the reaction mixture. The reaction was put under hydrogen pressure (80 psi) and stirred at room temperature for 16 hours. The reaction mixture was filtered through a celite bed and concentrated to obtain crude product. The crude product was washed with n-pentane and dried to afford tert-butyl 9-((1-(3-(2,6-dioxopiperidin-3-yl)-1-methyl-1H-indazol-7-yl)piperidin-4-yl)methyl)-3,9-diazaspiro[5.5]undecane-3-carboxylate (0.4 g) as a green solid. LC-MS (ESI): m/z=593.79 [M+H]⁺.

### Preparation of 3-(7-(4-((3,9-diazaspiro[5.5]undecan-3-yl)methyl)piperidin-1-yl)-1-methyl-1H-indazol-3-yl)piperidine-2,6-dione

To a cooled solution of tert-butyl 9-((1-(3-(2,6-dioxopiperidin-3-yl)-1-methyl-1H-indazol-7-yl)piperidin-4-yl)methyl)-3,9-diazaspiro[5.5]undecane-3-carboxylate (0.40 g, 0.67 mmol) in DCM (4.0 mL), TFA (4.0 mL) was added at 0° C. The reaction mixture was allowed to warm to room temperature over 2 hours with stirring. The reaction mixture was concentrated under reduced pressure to obtain crude product. The crude product was washed with diethyl ether to obtain 3-(7-(4-((3,9-diazaspiro[5.5]undecan-3-yl)methyl)piperidin-1-yl)-1-methyl-1H-indazol-3-yl)piperidine-2,6-dione (0.25 g) as a brown solid.

LC-MS (ESI): m/z=493.61 [M+H]⁺.

### Preparation of 2-((6-((5-chloro-2-(9-((1-(3-(2,6-dioxopiperidin-3-yl)-1-methyl-1H-indazol-7-yl)piperidin-4-yl)methyl)-3,9-diazaspiro[5.5]undecan-3-yl)pyrimidin-4-yl)amino)-1-methyl-2-oxo-1,2-dihydro-1,8-naphthyridin-3-yl)oxy)-N-methylacetamide

To a stirred solution of 3-(7-(4-((3,9-diazaspiro[5.5]undecan-3-yl)methyl)piperidin-1-yl)-1-methyl-1H-indazol-3-yl)piperidine-2,6-dione (0.15 g, 0.30 mmol) and 2-((6-((2,5-dichloropyrimidin-4-yl)amino)-1-methyl-2-oxo-1,2-dihydro-1,8-naphthyridin-3-yl)oxy)-N-methylacetamide (0.06 g, 0.15 mmol) in DMSO (3 mL) was added N,N-diisopropylethylamine (0.42 g, 2.43 mmol). The reaction mixture was stirred at 100° C. for 5 hours. The reaction mixture was poured into ice-cold water and stirred for 15 minutes. The resulting precipitate was filtered and dried to obtain crude product. The crude product was purified by prep-HPLC to afford 2-((6-((5-chloro-2-(9-((1-(3-(2,6-dioxopiperidin-3-yl)-1-methyl-1H-indazol-7-yl)piperidin-4-yl)methyl)-3,9-diazaspiro[5.5]undecan-3-yl)pyrimidin-4-yl)amino)-1-methyl-2-oxo-1,2-dihydro-1,8-naphthyridin-3-yl)oxy)-N-methylacetamide (0.009 g) as an off-white solid.

LC-MS (ESI): m/z=865.58 [M+H]⁺.

¹H NMR (400 MHz, DMSO-d₆): δ 10.87 (s, 1H), 9.03 (s, 1H), 8.73 (d, J=2.40 Hz, 1H), 8.28 (d, J=2.40 Hz, 1H), 8.07 (s, 1H), 7.95 (d, J=4.40 Hz, 1H), 7.36-7.34 (m, 1H), 7.15 (s, 1H), 7.00 (d, J=6.40 Hz, 2H), 4.62 (s, 2H), 4.33 (q, J=5.20 Hz, 1H), 0.00 (s, 3H), 3.74 (s, 3H), 3.60 (s, 4H), 3.29 (s, 2H), 2.64-2.62 (m, 7H), 2.53 (d, J=1.60 Hz, 4H), 2.31-2.30 (m, 3H), 2.15-2.17 (m, 1H), 1.85 (t, J=11.20 Hz, 2H), 1.73 (s, 1H), 1.53 (s, 4H), 1.42 (m, 6H).

### Example 17. Synthesis of 2-((6-((5-chloro-2-(7-((3-(2,6-dioxopiperidin-3-yl)-1-methyl-1H-indazol-7-yl)oxy)-2-azaspiro[3.5]nonan-2-yl)pyrimidin-4-yl)amino)-1-methyl-2-oxo-1,2-dihydro-1,8-naphthyridin-3-yl)oxy)-N-methylacetamide (Compound 142)

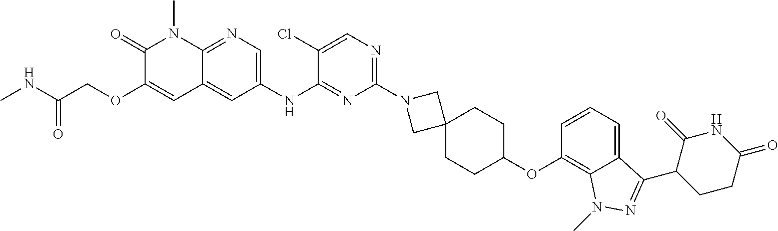 [SMILES: CNC(=O)COc1cc2cc(Nc3nc(N4CC5(CCC(Oc6cccc7c(C8CCC(=O)NC8=O)nn(C)c67)CC5)C4)ncc3Cl)cnc2n(C)c1=O]

### Preparation of 3-(2,6-bis(benzyloxy)pyridin-3-yl)-1-methyl-1H-indazol-7-ol

A stirred solution of 3-(2,6-bis(benzyloxy)pyridin-3-yl)-7-bromo-1-methyl-1H-indazole (1.00 g, 2.00 mmol) and KOH (0.78 g, 13.99 mmol) in a mixture of dioxane (25 mL) and water (5 mL) was degassed with argon for 5 minutes. T-butyl Xphos (0.17 g, 0.40 mmol) and Pd₂(dba)₃ (0.17 g, 0.20 mmol) was added, and the reaction was heated to 100° C. for 18 hours. The reaction mixture was diluted with ethyl acetate (300 mL) and filtered through a pad of celite. The collected filtrate was concentrated under vacuum to obtain crude product. The crude product was purified by flash column chromatography (230-400 mesh silica gel; 11% EtOAc in petroleum ether as eluent) to afford 3-(2,6-bis(benzyloxy)pyridin-3-yl)-1-methyl-1H-indazol-7-ol (0.67 g) as a pale green semi-solid.

LC-MS (ESI): m/z=438.46 [M+H]⁺.

### Preparation of tert-butyl 7-((methyl sulfonyl)oxy)-2-azaspiro[3.5]nonane-2-carboxylate

To a stirred solution of tert-butyl 7-hydroxy-2-azaspiro[3.5]nonane-2-carboxylate (0.50 g, 2.07 mmol) and TEA (0.87 mL, 6.22 mmol) in DCM (10 mL), MsCl (0.19 mL, 2.49 mmol) was added at 0° C. The reaction was allowed to warm to room temperature over 2 hours with stirring. The reaction mixture was quenched with saturated ammonium bicarbonate solution (10 mL) and extracted into DCM (3×30 mL). The organic layers were dried over anhydrous Na₂SO₄, filtered, and concentrated to obtain tert-butyl 7-((methyl sulfonyl)oxy)-2-azaspiro[3.5]nonane-2-carboxylate (0.66 g) as a pale-yellow solid.

LC-MS (ESI): m/z=320.19 [M+H]⁺.

### Preparation of tert-butyl 7-((3-(2,6-bis(benzyloxy)pyridin-3-yl)-1-methyl-1H-indazol-7-yl)oxy)-2-azaspiro[3.5]nonane-2-carboxylate

To stirred solution of tert-butyl 7-((methyl sulfonyl)oxy)-2-azaspiro[3.5]nonane-2-carboxylate (0.66 g, 2.07 mmol) and 3-(2,6-bis(benzyloxy)pyridin-3-yl)-1-methyl-1H-indazol-7-ol (0.50 g, 1.14 mmol) in DMF (6.6 mL), Cs₂CO₃ (2.02 g, 6.20 mmol) was added. The reaction was heated at 100° C. for 2 hours. The reaction mixture was quenched with water and extracted into ethyl acetate. The organic layers were washed with brine solution, dried over anhydrous Na₂SO₄, filtered, and concentrated to obtain tert-butyl 7-((3-(2,6-bis(benzyloxy)pyridin-3-yl)-1-methyl-1H-indazol-7-yl)oxy)-2-azaspiro[3.5]nonane-2-carboxylate (0.89 g) as a pale brown semi-solid.

LC-MS (ESI): m/z=661.67 [M+H]⁺.

### Preparation of tert-butyl 7-((3-(2,6-dioxopiperidin-3-yl)-1-methyl-1H-indazol-7-yl)oxy)-2-azaspiro[3.5]nonane-2-carboxylate

To a stirred solution of tert-butyl 7-((3-(2,6-bis(benzyloxy)pyridin-3-yl)-1-methyl-1H-indazol-7-yl)oxy)-2-azaspiro[3.5]nonane-2-carboxylate (0.89 g, 1.35 mmol) in THF (100 mL), 20% Pd (OH)₂ on carbon (1.13 g, 8.08 mmol) was added. The reaction was put under hydrogen atmosphere (80 psi) and stirred at room temperature for 18 hours. The reaction mixture was diluted with DCM (100 mL) and filtered through a pad of celite. The filtrate was concentrated under vacuum to afford tert-butyl 7-((3-(2,6-dioxopiperidin-3-yl)-1-methyl-1H-indazol-7-yl)oxy)-2-azaspiro[3.5]nonane-2-carboxylate (0.67 g) as a pale brown semi-solid. LC-MS (ESI): m/z=483.36 [M+H]⁺.

### Preparation of 3-(7-((2-azaspiro[3.5]nonan-7-yl)oxy)-1-methyl-1H-indazol-3-yl)piperidine-2,6-dione

To a stirred solution of tert-butyl 7-((3-(2,6-dioxopiperidin-3-yl)-1-methyl-1H-indazol-7-yl)oxy)-2-azaspiro[3.5]nonane-2-carboxylate (0.33 g, 0.68 mmol) in DCM (3.30 mL), TFA (2.64 mL) was added at 0° C. The reaction was allowed to warm to room temperature over 1 hour with stirring. The reaction mixture was concentrated under vacuum and co-distilled with petroleum ether (30 mL) to afford 3-(7-((2-azaspiro[3.5]nonan-7-yl)oxy)-1-methyl-1H-indazol-3-yl)piperidine-2,6-dione (0.55 g (TFA salt)) as a pale brown semi-solid. LC-MS (ESI): m/z=383.41 [M+H]⁺.

### Preparation of 2-((6-((5-chloro-2-(7-((3-(2,6-dioxopiperidin-3-yl)-1-methyl-1H-indazol-7-yl)oxy)-2-azaspiro[3.5]nonan-2-yl)pyrimidin-4-yl)amino)-1-methyl-2-oxo-1,2-dihydro-1,8-naphthyridin-3-yl)oxy)-N-methylacetamide

A stirred solution of 3-(7-((2-azaspiro[3.5]nonan-7-yl)oxy)-1-methyl-1H-indazol-3-yl)piperidine-2,6-dione (0.35 g, 0.91 mmol), 2-((6-((2,5-dichloropyrimidin-4-yl)amino)-1-methyl-2-oxo-1,2-dihydro-1,8-naphthyridin-3-yl)oxy)-N-methylacetamide (0.19 g, 0.46 mmol), DMSO (3.50 mL), and DIPEA (2.45 mL) was heated to 100° C. for 18 hours. Solvent was removed using a centrifugal evaporator to obtain crude product. The crude product was purified by preparative HPLC to afford 2-((6-((5-chloro-2-(7-((3-(2,6-dioxopiperidin-3-yl)-1-methyl-1H-indazol-7-yl)oxy)-2-azaspiro[3.5]nonan-2-yl)pyrimidin-4-yl)amino)-1-methyl-2-oxo-1,2-dihydro-1,8-naphthyridin-3-yl)oxy)-N-methylacetamide (0.051 g) as an off-white solid.

LC-MS (ESI): m/z=755.42 [M+H]⁺.

¹H NMR (400 MHz, DMSO-d₆): δ 10.87 (s, 1H), 9.03 (s, 1H), 8.82 (d, J=2.40 Hz, 1H), 8.42 (s, 1H), 8.07 (s, 1H), 7.95 (d, J=4.80 Hz, 1H), 7.22 (s, 1H), 7.19 (d, J=8.00 Hz, 1H), 6.96-6.98 (m, 1H), 6.88 (d, J=7.60 Hz, 1H), 4.62 (s, 3H), 4.29-4.30 (m, 1H), 4.18 (s, 3H), 3.74 (s, 7H), 2.66 (d, J=4.40 Hz, 3H), 2.60-2.61 (m, 2H), 2.29-2.30 (m, 1H), 2.13-2.15 (m, 1H), 1.93 (d, J=9.60 Hz, 4H), 1.73 (t, J=8.40 Hz, 4H).

### Example 18. Synthesis of 2-((6-((5-chloro-2-(4-(((1R,3s,5S)-3-(3-(2,6-dioxopiperidin-3-yl)-1-methyl-1H-indazol-7-yl)-8-azabicyclo[3.2.1]octan-8-yl)methyl)piperidin-1-yl)pyrimidin-4-yl)amino)-1-methyl-2-oxo-1,2-dihydro-1,8-naphthyridin-3-yl)oxy)-N-methylacetamide (Compound 143)

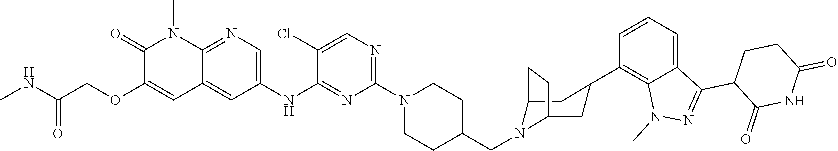 [SMILES: CNC(=O)COc1cc2cc(Nc3nc(N4CCC(CN5C6CCC5CC(c5cccc7c(C8CCC(=O)NC8=O)nn(C)c57)C6)CC4)ncc3Cl)cnc2n(C)c1=O]

### Preparation of tert-butyl (1s,5r)-3-(3-(2,6-bis(benzyloxy)pyridin-3-yl)-1-methyl-1H-indazol-7-yl)-8-azabicyclo[3.2.1]oct-2-ene-8-carboxylate

To a stirred solution of 3-(2,6-bis(benzyloxy)pyridin-3-yl)-7-bromo-1-methyl-1H-indazole (1.20 g, 2.40 mmol) and tert-butyl (1s,5r)-3-(4,4,5,5-tetramethyl-1,3,2-dioxaborolan-2-yl)-8-azabicyclo[3.2.1]oct-2-ene-8-carboxylate (0.96 g, 2.88 mmol) in 1,4-dioxane (9.6 mL) and water (2.4 mL) was added sodium carbonate (0.76 g, 7.20 mmol). The reaction was purged with N₂ for 10 minutes, Pd(dppf)Cl₂ (0.18 g, 0.24 mmol) was added, and the resultant reaction mixture was stirred at 100° C. for 16 hours. The reaction mixture was diluted with ethyl acetate (30 mL) and filtered through a celite bed. The celite was washed with 10% MeOH in DCM (100 mL). The filtrate was concentrated to obtain crude product. The crude product was purified by flash column (SiO₂, 230-400, 10-20% EtOAc in petroleum ether) to afford tert-butyl (1s,5r)-3-(3-(2,6-bis(benzyloxy)pyridin-3-yl)-1-methyl-1H-indazol-7-yl)-8-azabicyclo[3.2.1]oct-2-ene-8-carboxylate (1.02 g) as a yellow solid.

LC-MS (ESI): m/z=629.58 [M+H]⁺.

### Preparation of 5-(7-((1s,5r)-8-azabicyclo[3.2.1]oct-2-en-3-yl)-1-methyl-1H-indazol-3-yl)-6-(benzyloxy)pyridin-2-ol

To a stirred solution of tert-butyl (1s,5r)-3-(3-(2,6-bis(benzyloxy)pyridin-3-yl)-1-methyl-1H-indazol-7-yl)-8-azabicyclo[3.2.1]oct-2-ene-8-carboxylate (1.0 g, 1.60 mmol) in DCM (20.0 mL) was added 4 M HCl in dioxane (10.0 mL) at 0° C. The reaction was allowed to warm to room temperature over 16 hours with stirring. The reaction mixture was concentrated under vacuum to obtain crude product. The crude product was triturated with diethyl ether to afford 5-(7-((1s,5r)-8-azabicyclo[3.2.1]oct-2-en-3-yl)-1-methyl-1H-indazol-3-yl)-6-(benzyloxy)pyridin-2-ol (0.85 g) as an off-white solid.

LC-MS (ESI): m/z=439.29 [M+H]⁺.

### Preparation of tert-butyl 4-(((1s,5r)-3-(3-(2-(benzyloxy)-6-hydroxypyridin-3-yl)-1-methyl-1H-indazol-7-yl)-8-azabicyclo[3.2.1]oct-2-en-8-yl)methyl)piperidine-1-carboxylate

To a stirred solution of 5-(7-((1s,5r)-8-azabicyclo[3.2.1]oct-2-en-3-yl)-1-methyl-1H-indazol-3-yl)-6-(benzyloxy)pyridin-2-ol (0.70 g, 1.60 mmol) and tert-butyl 4-formylpiperidine-1-carboxylate (1.02 g, 4.80 mmol) in DCM (14.0 mL) was added triethylamine (1.10 mL, 7.98 mmol). The reaction was stirred at 40° C. for 2 hours. After cooling to 0° C., sodium triacetoxyborohydride (0.67 g, 3.19 mmol) was added, and the reaction mixture was allowed to warm to room temperature over 4 hours with stirring. The reaction mixture was quenched with NH₄Cl solution (30 mL) and extracted with DCM (2×50 mL). The combined organic layers were washed with brine solution, dried over anhydrous Na₂SO₄, filtered, and concentrated to obtain crude product. The crude was triturated with diethyl ether to afford tert-butyl 4-(((1s,5r)-3-(3-(2-(benzyloxy)-6-hydroxypyridin-3-yl)-1-methyl-1H-indazol-7-yl)-8-azabicyclo[3.2.1]oct-2-en-8-yl)methyl)piperidine-1-carboxylate (1.52 g) as an off-white solid. LC-MS (ESI): m/z=636.48 [M+H]⁺.

### Preparation of tert-butyl 4-(((1r,5s)-3-(3-(2,6-dioxopiperidin-3-yl)-1-methyl-1H-indazol-7-yl)-8-azabicyclo[3.2.1]octan-8-yl)methyl)piperidine-1-carboxylate

To a solution of tert-butyl 4-(((1s,5r)-3-(3-(2-(benzyloxy)-6-hydroxypyridin-3-yl)-1-methyl-1H-indazol-7-yl)-8-azabicyclo[3.2.1]oct-2-en-8-yl)methyl)piperidine-1-carboxylate (1.10 g, 1.73 mmol) in THF (66.0 mL) and acetic acid (0.1 mL, 1.73 mmol) was added 20% Pd (OH)₂/C (1.65 g, 150% w/w). The reaction was put under a hydrogen atmosphere (80 psi) at room temperature and stirred for 16 hours. The reaction mixture was diluted with THF (50 mL) and filtered through a celite bed. The celite was washed with THF: DCM (1:1, 200 mL). The filtrate was concentrated and dried to obtain tert-butyl 4-(((1r,5s)-3-(3-(2,6-dioxopiperidin-3-yl)-1-methyl-1H-indazol-7-yl)-8-azabicyclo[3.2.1]octan-8-yl)methyl)piperidine-1-carboxylate (0.65 g) as a pale yellow semi-solid.

LC-MS (ESI): m/z=550.46 [M+H]⁺.

### Preparation of 3-(1-methyl-7-((1r,5s)-8-(piperidin-4-ylmethyl)-8-azabicyclo[3.2.1]octan-3-yl)-1H-indazol-3-yl)piperidine-2,6-dione

To a stirred solution of tert-butyl 4-(((1r,5s)-3-(3-(2,6-dioxopiperidin-3-yl)-1-methyl-1H-indazol-7-yl)-8-azabicyclo[3.2.1]octan-8-yl)methyl)piperidine-1-carboxylate (0.61 g, 1.11 mmol) in DCM (12.2 mL), TFA (6.1 mL) was added at 0° C. The reaction mixture was allowed to warm to room temperature over 3 hours with stirring. The reaction mixture was concentrated under reduced pressure and co-distilled with DCM (2×10 mL) to obtain crude product. The crude product was triturated with diethyl ether to obtain 3-(1-methyl-7-((1r,5s)-8-(piperidin-4-ylmethyl)-8-azabicyclo[3.2.1]octan-3-yl)-1H-indazol-3-yl)piperidine-2,6-dione (0.51 g (TFA salt)) as a pale-yellow, gummy solid. LC-MS (ESI): m/z=450.31 [M+H]⁺.

### Preparation of 2-((6-((5-chloro-2-(4-(((1R,3s,5S)-3-(3-(2,6-dioxopiperidin-3-yl)-1-methyl-1H-indazol-7-yl)-8-azabicyclo[3.2.1]octan-8-yl)methyl)piperidin-1-yl)pyrimidin-4-yl)amino)-1-methyl-2-oxo-1,2-dihydro-1,8-naphthyridin-3-yl)oxy)-N-methylacetamide

To a stirred solution of 3-(1-methyl-7-((1r,5s)-8-(piperidin-4-ylmethyl)-8-azabicyclo[3.2.1]octan-3-yl)-1H-indazol-3-yl)piperidine-2,6-dione (0.30 g, 0.66 mmol) in DMSO (3.0 mL) and DIPEA (3.0 mL), 2-((6-((2,5-dichloropyrimidin-4-yl)amino)-1-methyl-2-oxo-1,2-dihydro-1,8-naphthyridin-3-yl)oxy)-N-methylacetamide (0.16 g, 0.40 mmol) was added. The reaction was stirred at 100° C. for 16 hours. Solvent was removed using a centrifugal evaporator to obtain crude product. The crude was purified by prep-HPLC to afford 2-((6-((5-chloro-2-(4-(((1R,3s,5S)-3-(3-(2,6-dioxopiperidin-3-yl)-1-methyl-1H-indazol-7-yl)-8-azabicyclo[3.2.1]octan-8-yl)methyl)piperidin-1-yl)pyrimidin-4-yl)amino)-1-methyl-2-oxo-1,2-dihydro-1,8-naphthyridin-3-yl)oxy)-N-methylacetamide (0.033 g) as an off-white solid. LC-MS (ESI): m/z=822.53 [M+H]⁺.

¹H NMR (400 MHz, DMSO-d₆): δ 10.91 (br s, 1H), 9.04 (br s, 1H), 8.26 (d, J=2.00 Hz, 1H), 8.26 (d, J=2.00 Hz, 1H), 8.07 (s, 1H), 7.92 (d, J=4.80 Hz, 1H), 7.48 (d, J=8.00 Hz, 1H), 7.25 (d, J=7.20 Hz, 1H), 7.17 (s, 1H), 7.04 (t, J=7.60 Hz, 1H), 4.60 (s, 2H), 4.46 (d, J=11.20 Hz, 2H), 4.31-4.32 (m, 1H), 4.15 (s, 3H), 3.93-3.81 (m, 1H), 3.74 (s, 3H), 3.29-3.21 (m, 2H), 2.87 (t, J=11.60 Hz, 2H), 2.69-2.63 (m, 5H), 2.50-2.33 (m, 3H), 2.15 (d, J=6.80 Hz, 3H), 1.98-1.96 (m, 2H), 1.83 (d, J=11.20 Hz, 2H), 1.65 (br s, 1H), 1.48-1.38 (d, J=7.60 Hz, 2H), 1.36 (t, J=12.40 Hz, 2H), 1.15-1.09 (m, 2H).

### Example 19. Synthesis of 2-((6-((5-chloro-2-(7-((3-(2,6-dioxopiperidin-3-yl)-1-methyl-1H-indazol-6-yl)oxy)-2-azaspiro[3.5]nonan-2-yl)pyrimidin-4-yl)amino)-1-methyl-2-oxo-1,2-dihydro-1,8-naphthyridin-3-yl)oxy)-N-methylacetamide (Compound 144)

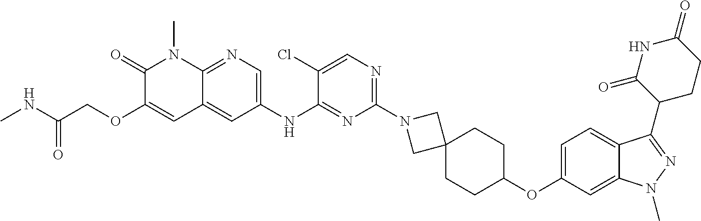 [SMILES: CNC(=O)COc1cc2cc(Nc3nc(N4CC5(CCC(Oc6ccc7c(C8CCC(=O)NC8=O)nn(C)c7c6)CC5)C4)ncc3Cl)cnc2n(C)c1=O]

### Preparation of 3-(2,6-bis(benzyloxy)pyridin-3-yl)-1-methyl-1H-indazol-6-ol

To a stirred solution of 3-(2,6-bis(benzyloxy)pyridin-3-yl)-6-bromo-1-methyl-1H-indazole (1.0 g, 2.00 mmol) in 1,4-dioxane (10.0 mL) and water (10.0 mL), potassium hydroxide (0.33 g, 5.99 mmol) was added. The reaction was purged with nitrogen for 15 minutes, tert-Butyl XPhos (0.10 g, 0.20 mmol) and Pd₂dba₃ (0.18 g, 0.20 mmol) were added, and the reaction was stirred at 100° C. 16 hours. The reaction mixture was diluted with water (100 mL) and extracted into ethyl acetate (2×100 mL). The combined organic layers were washed with brine solution, dried over anhydrous Na₂SO₄, filtered, and concentrated to obtain crude product. The crude product was purified by flash column chromatography (SiO₂, 60-120 mesh, 35% ethyl acetate in petroleum ether) to afford 3-(2,6-bis(benzyloxy)pyridin-3-yl)-1-methyl-1H-indazol-6-ol (0.7 g) as a yellow solid.

LC-MS (ESI): m/z=438.46 [M+H]⁺.

### Preparation of tert-butyl 7-((methylsulfonyl)oxy)-2-azaspiro[3.5]nonane-2-carboxylate

A stirred solution of tert-butyl 7-hydroxy-2-azaspiro[3.5]nonane-2-carboxylate (0.7 g, 2.9 mmol) and TEA (0.83 mL, 5.8 mmol) in DCM (14.0 mL) was cooled to 0° C. Methanesulfonyl chloride (0.39 g, 3.48 mmol) was added and the reaction was allowed to warm to room temperature over 4 hours with stirring. The reaction mixture was diluted with water (100 mL) and extracted into DCM (2×100 mL). The combined organic layers were washed with brine solution, dried over anhydrous Na₂SO₄, filtered, and concentrated under reduced pressure to isolate crude product. The crude product was purified by trituration using diethyl ether to afford tert-butyl 7-((methylsulfonyl)oxy)-2-azaspiro[3.5]nonane-2-carboxylate (0.7 g) as a yellow semi-solid.

¹H NMR (400 MHz, CDCl₃): δ 4.72 (m, 1H), 3.61 (m, 2H), 3.01 (m, 2H), 3.01 (s, 3H), 1.93 (m, 4H), 1.65 (m, 4H), 1.44 (s, 9H).

### Preparation of tert-butyl 7-((3-(2,6-bis(benzyloxy)pyridin-3-yl)-1-methyl-1H-indazol-6-yl)oxy)-2-azaspiro[3.5]nonane-2-carboxylate

To a stirred solution of 3-(2,6-bis(benzyloxy)pyridin-3-yl)-1-methyl-1H-indazol-6-ol (0.7 g, 1.60 mmol) and tert-butyl 7-((methylsulfonyl)oxy)-2-azaspiro[3.5]nonane-2-carboxylate (0.61 g, 1.92 mmol) in DMF (14.0 mL) at 0° C., Cs₂CO₃ (1.56 g, 4.80 mmol) was added. The reaction was then stirred at 100° C. for 16 hours. The reaction mixture was diluted with water (70 mL) and extracted into ethyl acetate (150 mL). The combined organic layers were washed with brine solution, dried over anhydrous Na₂SO₄, filtered, and concentrated to obtain crude product. The crude product was purified by flash column chromatography (SiO₂, 60-120 mesh, 20% ethyl acetate in petroleum ether) to afford tert-butyl 7-((3-(2,6-bis(benzyloxy)pyridin-3-yl)-1-methyl-1H-indazol-6-yl)oxy)-2-azaspiro[3.5]nonane-2-carboxylate (0.8 g) as a yellow solid. LC-MS (ESI): m/z=661.73 [M+H]⁺.

### Preparation of tert-butyl 7-((3-(2,6-dioxopiperidin-3-yl)-1-methyl-1H-indazol-6-yl)oxy)-2-azaspiro[3.5]nonane-2-carboxylate

To a stirred solution of tert-butyl 7-((3-(2,6-bis(benzyloxy)pyridin-3-yl)-1-methyl-1H-indazol-6-yl)oxy)-2-azaspiro[3.5]nonane-2-carboxylate (0.7 g, 1.06 mmol) in THF (21 mL), 20% Pd (OH)₂ on carbon (1.4 g) was added. The reaction was put under hydrogen atmosphere (20 psi) at room temperature and stirred for 4 hours. The reaction mixture was diluted with EtOAc (100 mL) and filtered through a celite bed. The celite was washed with EtOAc (2×50 mL). The collected filtrate was concentrated under reduced pressure to obtain crude product. The crude product was triturated with diethyl ether to afford tert-butyl 7-((3-(2,6-dioxopiperidin-3-yl)-1-methyl-1H-indazol-6-yl)oxy)-2-azaspiro[3.5]nonane-2-carboxylate (0.5 g) as an off-white solid.

LC-MS (ESI): m/z=483.53 [M+Na]⁺.

### Preparation of 3-(6-((2-azaspiro[3.5]nonan-7-yl)oxy)-1-methyl-1H-indazol-3-yl)piperidine-2,6-dione

To a stirred solution of tert-butyl 7-((3-(2,6-dioxopiperidin-3-yl)-1-methyl-1H-indazol-6-yl)oxy)-2-azaspiro[3.5]nonane-2-carboxylate (0.5 g, 1.09 mmol) in DCM (5 mL) was added TFA (2.5 mL). After stirring at room temperature for 3 hours, the reaction mixture was concentrated under reduced pressure to obtain crude product. The crude product was triturated using diethyl ether to afford 3-(6-((2-azaspiro[3.5]nonan-7-yl)oxy)-1-methyl-1H-indazol-3-yl)piperidine-2,6-dione (0.4 g (TFA salt)). LC-MS (ESI): m/z=383.51 [M+H]⁺.

### Preparation of 2-((6-((5-chloro-2-(7-((3-(2,6-dioxopiperidin-3-yl)-1-methyl-1H-indazol-6-yl)oxy)-2-azaspiro[3.5]nonan-2-yl)pyrimidin-4-yl)amino)-1-methyl-2-oxo-1,2-dihydro-1,8-naphthyridin-3-yl)oxy)-N-methylacetamide

To a stirred solution of 3-(6-((2-azaspiro[3.5]nonan-7-yl)oxy)-1-methyl-1H-indazol-3-yl)piperidine-2,6-dione (TFA salt) (0.2 g, 0.41 mmol) in DMSO (4 mL), 2-((6-((2,5-dichloropyrimidin-4-yl)amino)-1-methyl-2-oxo-1,2-dihydro-1,8-naphthyridin-3-yl)oxy)-N-methylacetamide (0.067 g, 0.16 mmol) and DIPEA (0.38 mL, 2.0 mmol) were added. The reaction was heated to 100° C. for 4 hour. The solvent was removed using a centrifugal evaporator to obtain crude product. The crude product was purified by preparative HPLC to afford 2-((6-((5-chloro-2-(7-((3-(2,6-dioxopiperidin-3-yl)-1-methyl-1H-indazol-6-yl)oxy)-2-azaspiro[3.5]nonan-2-yl)pyrimidin-4-yl)amino)-1-methyl-2-oxo-1,2-dihydro-1,8-naphthyridin-3-yl)oxy)-N-methylacetamide (0.051 g) as off-white solid.

LC-MS (ESI): m/z=755.43 [M+H]⁺.

¹H NMR (400 MHz, DMSO-d₆): δ 10.85 (s, 1H), 8.82-8.81 (m, 1H), 8.42-8.41 (m, 1H), 8.06 (s, 1H), 7.96-7.95 (m, 1H), 7.56-7.54 (m, 1H), 7.23 (s, 1H), 7.09-7.08 (m, 1H), 6.75-6.72 (m, 1H), 4.62 (s, 2H), 4.30-4.27 (m, 1H), 3.91 (s, 3H), 3.74-3.71 (m, 6H), 2.68-2.50 (m, 5H), 2.32-2.28 (m, 1H), 2.18-2.13 (m, 1H), 1.93-1.91 (m, 4H), 1.79 (s, 3H), 1.70-1.59 (m, 3H).

### Example 20. Synthesis of 2-((6-((5-chloro-2-(4-((3-(2,6-dioxopiperidin-3-yl)-1-methyl-1H-indazol-6-yl)oxy)piperidin-1-yl)pyrimidin-4-yl)amino)-1-methyl-2-oxo-1,2-dihydro-1,8-naphthyridin-3-yl)oxy)-N-methylacetamide (Compound 147)

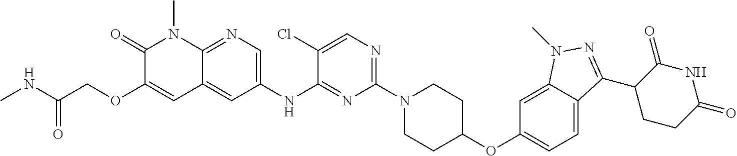 [SMILES: CNC(=O)COc1cc2cc(Nc3nc(N4CCC(Oc5ccc6c(C7CCC(=O)NC7=O)nn(C)c6c5)CC4)ncc3Cl)cnc2n(C)c1=O]

### Preparation of tert-butyl 4-[3-(2,6-dibenzyloxy-3-pyridyl)-1-methyl-indazol-6-yl]oxypiperidine-1-carboxylate

A mixture of 3-(2,6-dibenzyloxy-3-pyridyl)-1-methyl-indazol-6-ol (5.5 g, 12.6 mmol, 1 eq.), tert-butyl 4-hydroxypiperidine-1-carboxylate (5.06 g, 25.1 mmol, 2 eq.), 2-(tributyl-25-phosphanylidene) acetonitrile (3.34 g, 13.8 mmol, 1.1 eq.) in toluene (100 mL) was degassed and purged with N₂ 3 times. The reaction mixture was stirred at 110° C. for 16 hours under N₂ atmosphere. The reaction mixture was concentrated under reduced pressure to give a residue. The residue was purified by column chromatography (SiO₂, petroleum ether/ethyl acetate=20/1 to 2/1) to afford tert-butyl 4-[3-(2,6-dibenzyloxy-3-pyridyl)-1-methyl-indazol-6-yl]oxypiperidine-1-carboxylate (16 g) as a yellow oil.

¹H NMR (400 MHz, DMSO-d₆) ô=7.89 (d, J=8.0 Hz, 1H), 7.54 (d, J=8.8 Hz, 1H), 7.50-7.45 (m, 2H), 7.43-7.24 (m, 8H), 7.13 (d, J=1.6 Hz, 1H), 6.66 (dd, J=2.0, 8.8 Hz, 1H), 6.58 (d, J=8.0 Hz, 1H), 5.48-5.40 (m, 4H), 4.70-4.64 (m, 1H), 4.00 (s, 3H), 3.30-3.15 (m, 2H), 2.95 (s, 3H), 2.03-1.92 (m, 2H), 1.60-1.53 (m, 2H), 1.39 (s, 9H).

### Preparation of tert-butyl 4-[3-(2,6-dioxo-3-piperidyl)-1-methyl-indazol-6-yl]oxypiperidine-1-carboxylate

A mixture of tert-butyl 4-[3-(2,6-dibenzyloxy-3-pyridyl)-1-methyl-indazol-6-yl]oxypiperidine-1-carboxylate (16 g, 25.8 mmol, 1 eq.), Pd/C (3 g, 25.8 mmol, 10% purity, 1 eq.), AcOH (1.55 g, 25.8 mmol, 1.48 mL, 1 eq.), Pd (OH)₂ (3 g, 2.14 mmol, 10% purity, 8.29e-2 eq.) and EtOH (100 mL) in THF (100 mL) was degassed and purged with H₂ (50 psi)₃ times. The mixture was stirred at 80° C. for 16 hours under H₂ (50 psi) atmosphere. The reaction mixture was filtered and concentrated under reduced pressure to give a residue. The residue was purified by reverse-phase HPLC (0.1% TFA condition) to afford tert-butyl 4-[3-(2,6-dioxo-3-piperidyl)-1-methyl-indazol-6-yl]oxypiperidine-1-carboxylate (3.8 g) as a colorless oil.

¹H NMR (400 MHz, DMSO-d₆) ¿=10.87 (s, 1H), 7.57 (d, J=8.8 Hz, 1H), 7.13 (d, J=1.6 Hz, 1H), 6.77 (dd, J=1.6, 8.8 Hz, 1H), 4.74-4.65 (m, 1H), 4.30 (dd, J=5.2, 9.6 Hz, 1H), 3.93 (s, 3H), 3.74-3.63 (m, 2H), 3.29-3.16 (m, 2H), 2.71-2.56 (m, 2H), 2.39-2.27 (m, 1H), 2.21-2.12 (m, 1H), 2.02-1.92 (m, 2H), 1.64-1.52 (m, 2H), 1.42 (s, 9H).

### Preparation of 3-[1-methyl-6-(4-piperidyloxy) indazol-3-yl]piperidine-2,6-dione

A solution of tert-butyl 4-[3-(2,6-dioxo-3-piperidyl)-1-methyl-indazol-6-yl]oxypiperidine-1-carboxylate (3.8 g, 8.59 mmol, 1 eq.) in HCl/dioxane (30 mL) and DCM (10 mL) was stirred at 25° C. for 1 hour. The reaction mixture was filtered to give a filter cake. The filter cake was triturated with MeCN at 25° C. for 10 minutes to afford 3-[1-methyl-6-(4-piperidyloxy) indazol-3-yl]piperidine-2,6-dione (2.20 g) as a white solid.

LC-MS (ESI): m/z=343.2 [M+H]⁺.

### Preparation of 2-((6-((5-chloro-2-(4-((3-(2,6-dioxopiperidin-3-yl)-1-methyl-1H-indazol-6-yl)oxy)piperidin-1-yl)pyrimidin-4-yl)amino)-1-methyl-2-oxo-1,2-dihydro-1,8-naphthyridin-3-yl)oxy)-N-methylacetamide

A mixture of 2-((6-((2,5-dichloropyrimidin-4-yl)amino)-1-methyl-2-oxo-1,2-dihydro-1,8-naphthyridin-3-yl)oxy)-N-methylacetamide (40.0 mg, 1.00 eq., 97.7 μmol) and 3-(1-methyl-6-(piperidin-4-yloxy)-1H-indazol-3-yl)piperidine-2,6-dione, HCl (44.4 mg, 1.20 eq., 117 μmol) in DMSO (0.700 mL) was treated with DIEA (50.5 mg, 68.1 μL, 4.00 eq., 391 μmol) and warmed to 80° C. for 19 hours. The reaction mixture was cooled to room temperature, filtered, and purified by prep-HPLC to afford 2-((6-((5-chloro-2-(4-((3-(2,6-dioxopiperidin-3-yl)-1-methyl-1H-indazol-6-yl)oxy)piperidin-1-yl)pyrimidin-4-yl)amino)-1-methyl-2-oxo-1,2-dihydro-1,8-naphthyridin-3-yl)oxy)-N-methylacetamide (28.5 mg).

UPLC-MS (ESI): m/z=715.3 [M+H]⁺.

¹H NMR (400 MHz, DMSO) & 10.85 (s, 1H), 9.10 (s, 1H), 8.72 (d, J=2.4 Hz, 1H), 8.29 (d, J=2.5 Hz, 1H), 8.11 (s, 1H), 7.90 (q, J=4.7 Hz, 1H), 7.56 (d, J=8.8 Hz, 1H), 7.22-7.07 (m, 2H), 6.76 (dd, J=8.8, 2.0 Hz, 1H), 4.84-4.70 (m, 1H), 4.59 (s, 2H), 4.29 (dd, J=9.5, 5.1 Hz, 1H), 4.05 (d, J=13.0 Hz, 2H), 3.92 (s, 3H), 3.72 (s, 3H), 3.50 (t, J=9.6 Hz, 2H), 2.70-2.61 (m, 2H), 2.59 (d, J=4.7 Hz, 3H), 2.32 (dtd, J=14.3, 9.3, 5.3 Hz, 1H), 2.16 (dq, J=13.2, 5.5 Hz, 1H), 2.01 (s, 2H), 1.65 (dd, J=8.6, 4.4 Hz, 1H).

### Example 21. Synthesis of 2-((6-((5-chloro-2-(4-((1-(3-(2,6-dioxopiperidin-3-yl)-1-methyl-1H-indazol-7-yl)piperidin-4-yl)methyl)-1,4-diazepan-1-yl)pyrimidin-4-yl)amino)-1-methyl-2-oxo-1,2-dihydro-1,8-naphthyridin-3-yl)oxy)-N-methylacetamide (Compound 110)

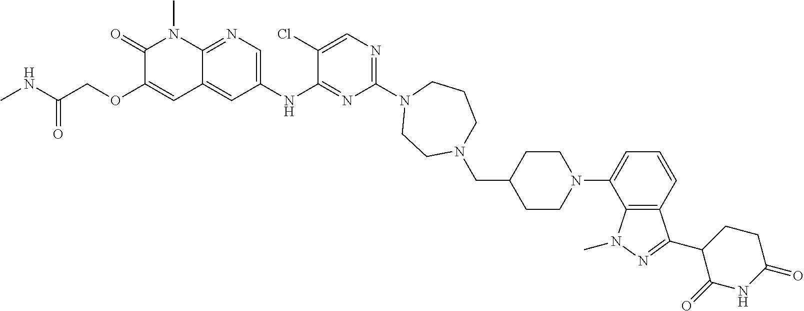 [SMILES: CNC(=O)COc1cc2cc(Nc3nc(N4CCCN(CC5CCN(c6cccc7c(C8CCC(=O)NC8=O)nn(C)c67)CC5)CC4)ncc3Cl)cnc2n(C)c1=O]

### Preparation of tert-butyl 4-((1-((benzyloxy) carbonyl)piperidin-4-yl)methyl)-1,4-diazepane-1-carboxylate

To a stirred solution of tert-butyl 1,4-diazepane-1-carboxylate (4.00 g, 20.0 mmol) and benzyl 4-formylpiperidine-1-carboxylate (4.93 g, 20.0 mmol) in DCM (40 mL), acetic acid (1.14 mL, 20.0 mmol) was added. After stirring for 1 hour, sodium triacetoxyborohydride (8.46 g, 39.9 mmol) was added, and the reaction was stirred at room temperature for 16 hours. The reaction mixture was quenched with ammonium chloride solution (100 mL) and extracted with DCM (2×100 mL). The combined organic layers were washed with brine solution, dried over anhydrous Na₂SO₄, filtered, and concentrated under reduced pressure to afford tert-butyl 4-((1-((benzyloxy) carbonyl)piperidin-4-yl)methyl)-1,4-diazepane-1-carboxylate (5.1 g) as a pale yellow, gummy solid.

LC-MS (ESI): m/z=432.32 [M+H]⁺.

### Preparation of tert-butyl 4-(piperidin-4-ylmethyl)-1,4-diazepane-1-carboxylate

To a solution of tert-butyl 4-((1-((benzyloxy) carbonyl)piperidin-4-yl)methyl)-1,4-diazepane-1-carboxylate (3.00 g, 6.95 mmol) in THF (120 mL), 20% Pd (OH)₂/C (1.50 g) was added. The reaction was put under hydrogen atmosphere (80 psi) and stirred at room temperature for 16 hours. The reaction mixture was diluted with THF (50 mL) and filtered through a celite bed. The celite was washed with 200 mL of THF: DCM (1:1) and the filtrate was concentrated to afford tert-butyl 4-(piperidin-4-ylmethyl)-1,4-diazepane-1-carboxylate (1.8 g) as a brown gummy solid.

¹H NMR (400 MHz, CDCl₃): δ 3.48-3.39 (m, 4H), 3.08 (d, J=12.00 Hz, 2H), 2.65-2.56 (m, 6H), 2.28-2.29 (m, 2H), 1.79-1.73 (m, 4H), 1.43 (s, 9H), 1.12-1.03 (m, 2H).

### Preparation of tert-butyl 4-((1-(3-(2,6-bis(benzyloxy)pyridin-3-yl)-1-methyl-1H-indazol-7-yl)piperidin-4-yl)methyl)-1,4-diazepane-1-carboxylate

To a stirred solution of 3-(2,6-bis(benzyloxy)pyridin-3-yl)-7-bromo-1-methyl-1H-indazole (0.50 g, 0.99 mmol) and tert-butyl 4-(piperidin-4-ylmethyl)-1,4-diazepane-1-carboxylate (0.89 g, 2.99 mmol) in 1,4-dioxane (10 mL), cesium carbonate (0.97 g, 2.99 mmol) was added. The reaction was degassed with argon for 15 minutes. Pd-PEPPSI-IHeptCl (0.048 g, 0.05 mmol) was added, and the reaction was stirred at 100° C. for 16 hours. The reaction mixture was diluted with ethyl acetate (40 mL) and filtered through a celite bed. The celite was washed with 10% MeOH: DCM (250 mL) and the filtrate was concentrated to obtain crude product. The crude product was purified by flash chromatography (230-400 silica gel; 10-15% EtOAc in petroleum ether) to afford tert-butyl 4-((1-(3-(2,6-bis(benzyloxy)pyridin-3-yl)-1-methyl-1H-indazol-7-yl)piperidin-4-yl)methyl)-1,4-diazepane-1-carboxylate (0.33 g) as a pale yellow solid. LC-MS (ESI): m/z=717.71 [M+H]⁺.

### Preparation of tert-butyl 4-((1-(3-(2,6-dioxopiperidin-3-yl)-1-methyl-1H-indazol-7-yl)piperidin-4-yl)methyl)-1,4-diazepane-1-carboxylate

To a solution of tert-butyl 4-((1-(3-(2,6-bis(benzyloxy)pyridin-3-yl)-1-methyl-1H-indazol-7-yl)piperidin-4-yl)methyl)-1,4-diazepane-1-carboxylate (0.33 g, 0.46 mmol) in THF (32 mL) was added 20% Pd (OH)₂/C (0.66 g). The reaction was put under hydrogen atmosphere (80 psi) and stirred at room temperature for 5 hours. The reaction mixture was diluted with THF (60 mL) and filtered through a celite bed. The celite was washed with THF: DCM (1:1, 200 mL) and the filtrate was concentrated to afford tert-butyl 4-((1-(3-(2,6-dioxopiperidin-3-yl)-1-methyl-1H-indazol-7-yl)piperidin-4-yl)methyl)-1,4-diazepane-1-carboxylate (0.22 g) as a brown solid.

LC-MS (ESI): m/z=551.68 [M+H]⁺.

### Preparation of 3-(7-(4-((1,4-diazepan-1-yl)methyl)piperidin-1-yl)-1-methyl-1H-indazol-3-yl)piperidine-2,6-dione

To a stirred solution of tert-butyl 4-((1-(3-(2,6-dioxopiperidin-3-yl)-1-methyl-1H-indazol-7-yl)piperidin-4-yl)methyl)-1,4-diazepane-1-carboxylate (0.59 g, 1.07 mmol) in DCM (6 mL), TFA (3.0 mL) was added at 0° C. The reaction was allowed to warm to room temperature over 3 hours with stirring. The reaction mixture was concentrated under reduced pressure and co-distilled with DCM (3×10 mL) to afford 3-(7-(4-((1,4-diazepan-1-yl)methyl)piperidin-1-yl)-1-methyl-1H-indazol-3-yl)piperidine-2,6-dione (0.25 g) as a brown solid.

LC-MS (ESI): m/z=451.29 [M+H]⁺.

### Preparation of 2-((6-((5-chloro-2-(4-((1-(3-(2,6-dioxopiperidin-3-yl)-1-methyl-1H-indazol-7-yl)piperidin-4-yl)methyl)-1,4-diazepan-1-yl)pyrimidin-4-yl)amino)-1-methyl-2-oxo-1,2-dihydro-1,8-naphthyridin-3-yl)oxy)-N-methylacetamide

To a stirred solution of 3-(7-(4-((1,4-diazepan-1-yl)methyl)piperidin-1-yl)-1-methyl-1H-indazol-3-yl)piperidine-2,6-dione (0.12 g, 0.27 mmol) in DMSO (1.2 mL), DIPEA (0.96 mL) and 2-((6-((2,5-dichloropyrimidin-4-yl)amino)-1-methyl-2-oxo-1,2-dihydro-1,8-naphthyridin-3-yl)oxy)-N-methylacetamide (0.04 g, 0.10 mmol) were added. The reaction was then stirred at 100° C. for 16 hours. The solvent was removed using a centrifugal evaporator to obtain crude product. The crude product was purified via prep-HPLC to afford 2-((6-((5-chloro-2-(4-((1-(3-(2,6-dioxopiperidin-3-yl)-1-methyl-1H-indazol-7-yl)piperidin-4-yl)methyl)-1,4-diazepan-1-yl)pyrimidin-4-yl)amino)-1-methyl-2-oxo-1,2-dihydro-1,8-naphthyridin-3-yl)oxy)-N-methylacetamide (0.023 g) as an off-white solid.

LC-MS (ESI): m/z=811.54 [M+H]⁺.

¹H NMR (400 MHz, DMSO-d₆): δ 10.87 (s, 1H), 9.02 (s, 1H), 8.74 (d, J=2.00 Hz, 1H), 8.39 (br s, 3H), 7.96 (d, J=4.40 Hz, 1H), 7.36 (d, J=8.40 Hz, 1H), 7.13 (br s, 1H), 7.02-6.99 (m, 2H), 4.61 (s, 2H), 4.32 (q, J=5.20 Hz, 1H), 4.21 (s, 1H), 3.73 (s, 8H), 3.18 (s, 2H), 2.71-2.58 (m, 10H), 2.36-2.33 (m, 3H), 2.18-2.12 (m, 1H), 1.89 (s, 4H), 1.57 (s, 1H), 1.36-1.21 (m, 3H).

### Example 22:2-((6-((5-chloro-2-((3R)-3-(((3-(2,6-dioxopiperidin-3-yl)-1-methyl-1H-indazol-7-yl)amino)methyl) pyrrolidin-1-yl)pyrimidin-4-yl)amino)-1-methyl-2-oxo-1,2-dihydro-1,8-naphthyridin-3-yl)oxy)-N-methylacetamide (Compound 122a)

### Preparation of tert-butyl (R)-3-(((3-(2,6-bis(benzyloxy)pyridin-3-yl)-1-methyl-1H-indazol-7-yl)amino)methyl) pyrrolidine-1-carboxylate

To a stirred solution of tert-butyl (R)-3-(aminomethyl) pyrrolidine-1-carboxylate (0.7 g, 3.51 mmol) and 3-(2,6-bis(benzyloxy)pyridin-3-yl)-7-bromo-1-methyl-1H-indazole (1.0 g, 2.11 mmol) in 1,4-dioxane (14.0 mL) was added sodium tert-butoxide (1.0 g, 10.5 mmol). The reaction mixture was purged for 15 minutes with nitrogen followed by the addition of Pd-PEPPSI-iHeptCl (0.17 g, 0.17 mmol). The resulting reaction mixture was stirred and heated at 100° C. for 3 h. After completion, the reaction mixture was cooled to room temperature and filtered through a celite bed and washed with EtOAc (15 mL). The filtrate was collected and concentrated under reduced pressure to afford a crude product which was purified by flash column chromatography (SiO₂, 100-200 mesh; 30% ethyl acetate in petroleum ether) to obtain tert-butyl (R)-3-(((3-(2,6-bis(benzyloxy)pyridin-3-yl)-1-methyl-1H-indazol-7-yl)amino)methyl) pyrrolidine-1-carboxylate (0.5 g) as an off white solid.

LC-MS (ESI): 620.71 [M+H]⁺

### Preparation of tert-butyl (3R)-3-(((3-(2,6-dioxopiperidin-3-yl)-1-methyl-1H-indazol-7-yl) amino)methyl) pyrrolidine-1-carboxylate

A stirred solution of tert-butyl (R)-3-(((3-(2,6-bis(benzyloxy)pyridin-3-yl)-1-methyl-1H-indazol-7-yl)amino)methyl) pyrrolidine-1-carboxylate (0.5 g, 0.81 mmol) in a mixture of THF (15 mL) and ethyl acetate (15 mL) at room temperature was degassed with nitrogen for 5 minutes. 10% Pd/C (0.51 g, 4.84 mmol) was then added, and the resulting reaction mixture was stirred under hydrogen atmosphere (80 psi) pressure for 16 h. After completion, the reaction mixture was diluted with ethyl acetate (50 mL), filtered through a pad of celite, and concentrated under reduced pressure to afford a crude product which was purified by trituration with n-pentane (2.0 ml) to afford tert-butyl (3R)-3-(((3-(2,6-dioxopiperidin-3-yl)-1-methyl-1H-indazol-7-yl)amino)methyl) pyrrolidine-1-carboxylate (0.3 g) as a brown gum. LC-MS (ESI): m/z=464.50 [M+Na]⁺

### Preparation of 3-(1-methyl-7-((((S)-pyrrolidin-3-yl)methyl) amino)-1H-indazol-3-yl) piperidine-2,6-dione hydrochloride

To a stirred solution of tert-butyl (3R)-3-(((3-(2,6-dioxopiperidin-3-yl)-1-methyl-1H-indazol-7-yl)amino)methyl) pyrrolidine-1-carboxylate (0.3 g, 0.68 mmol) in 1,4-dioxane (3.0 mL) was added 4M HCl in 1,4-dioxane (3.0 mL) at 0° C. under N₂ atmosphere. The reaction mixture was stirred at room temperature for 3 h. After completion, the reaction mixture was evaporated under reduced pressure to provide a crude product which was purified by trituration with n-pentane (10 ml) to afford 3-(1-methyl-7-((((S)-pyrrolidin-3-yl)methyl) amino)-1H-indazol-3-yl)piperidine-2,6-dione hydrochloride (0.2 g) as a brown gum.

LC-MS (ESI): m/z=342.44 [M+H]⁺

### Preparation of 2-((6-((5-chloro-2-((3R)-3-(((3-(2,6-dioxopiperidin-3-yl)-1-methyl-1H-indazol-7-yl)amino)methyl) pyrrolidin-1-yl)pyrimidin-4-yl)amino)-1-methyl-2-oxo-1,2-dihydro-1,8-naphthyridin-3-yl)oxy)-N-methylacetamide

To a stirred solution of 3-(1-methyl-7-((((S)-pyrrolidin-3-yl)methyl) amino)-1H-indazol-3-yl) piperidine-2,6-dione hydrochloride (0.2 g, 0.53 mmol) in DMSO (4.0 mL) was added DIPEA (0.46 mL, 2.65 mmol) and 2-((6-((2,5-dichloropyrimidin-4-yl)amino)-1-methyl-2-oxo-1,2-dihydro-1,8-naphthyridin-3-yl)oxy)-N-methylacetamide (0.08 g, 0.21 mmol) at room temperature under N₂ atmosphere. The reaction mixture was then stirred and heated at 100° C. for 3 h. After completion, the reaction mixture was cooled to room temperature and purified by prep-HPLC to afford 2-((6-((5-chloro-2-((3R)-3-(((3-(2,6-dioxopiperidin-3-yl)-1-methyl-1H-indazol-7-yl)amino)methyl) pyrrolidin-1-yl)pyrimidin-4-yl)amino)-1-methyl-2-oxo-1,2-dihydro-1,8-naphthyridin-3-yl)oxy)-N-methylacetamide (0.009 g) as a brown solid. LC-MS (ESI): m/z=714.18 [M+H]⁺

¹H NMR (400 MHz, DMSO-d₆): δ 10.85 (s, 1H), 8.98 (s, 1H), 8.86 (br s, 1H), 8.48 (br s, 1H), 8.07 (s, 1H), 7.94 (br s, 1H), 7.21 (s, 1H), 6.97-6.95 (m, 1H), 6.90-6.88 (m, 1H), 6.51 (d, J=7.2 Hz, 1H), 5.45 (br s, 1H), 4.59 (br s, 1H), 4.27-4.24 (m, 4H), 3.71 (br s, 5H), 3.58 (s, 2H), 2.72 (br s, 2H), 2.72-2.58 (m, 6H), 2.32-2.27 (m, 1H), 2.16-2.11 (m, 2H), 1.83-1.82 (m, 1H)

### Example 23:2-((6-((5-chloro-2-((3S)-3-(((3-(2,6-dioxopiperidin-3-yl)-1-methyl-1H-indazol-7-yl)amino)methyl) pyrrolidin-1-yl)pyrimidin-4-yl)amino)-1-methyl-2-oxo-1,2-dihydro-1,8-naphthyridin-3-yl)oxy)-N-methylacetamide (Compound 122b)

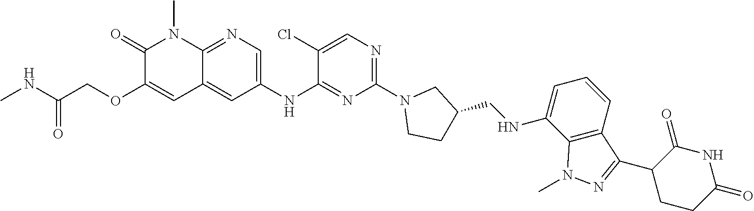 [SMILES: CNC(=O)COc1cc2cc(Nc3nc(N4CC[C@@H](CNc5cccc6c(C7CCC(=O)NC7=O)nn(C)c56)C4)ncc3Cl)cnc2n(C)c1=O]

### Preparation of tert-butyl-(S)-3-(((3-(2,6-bis(benzyloxy)pyridin-3-yl)-1-methyl-1H-indazol-7-yl)amino)methyl) pyrrolidine-1-carboxylate

A stirred suspension of 3-(2,6-bis(benzyloxy)pyridin-3-yl)-7-bromo-1-methyl-1H-indazole (0.70 g, 1.40 mmol) and tert-butyl(S)-3-(aminomethyl) pyrrolidine-1-carboxylate (0.44 g, 2.24 mmol), cesium carbonate (1.36 g, 4.19 mmol) in 1,4-dioxane (14.0 mL) was purged with argon for 15 minutes. Pd PEPPSI-iHept-Cl (0.06 g, 0.007 mmol) was then added, and the reaction mixture was stirred and heated at 100° C. for 3 h. The reaction mixture was quenched with water (30 mL) and extracted with ethyl acetate (3×50 mL). The combined organic layers were washed with brine (20 mL), dried over anhydrous Na₂SO₄, filtered, and concentrated to provide a crude product which was purified by flash column chromatography (25% ethyl acetate in petroleum ether as eluent) to afford tert-butyl-(S)-3-(((3-(2,6-bis(benzyloxy)pyridin-3-yl)-1-methyl-1H-indazol-7-yl)amino)methyl) pyrrolidine-1-carboxylate (0.60 g) as a brown semi solid.

LC-MS (ESI): m/z=620.72 [M+H]⁺

### Preparation of tert-butyl (3S)-3-(((3-(2,6-dioxopiperidin-3-yl)-1-methyl-1H-indazol-7-yl)amino)methyl) pyrrolidine-1-carboxylate

To a stirred solution of tert-butyl(S)-3-(((3-(2,6-bis(benzyloxy)pyridin-3-yl)-1-methyl-1H-indazol-7-yl)amino)methyl) pyrrolidine-1-carboxylate (0.60 g, 0.97 mmol) in ethyl acetate (36.0 mL) was added 10% Pd/C (0.60 g, 100% w/w). The reaction mixture was stirred under hydrogen atmosphere (15 psi) for 24 h at room temperature. The reaction mixture was then filtered through a celite bed using 50% THF-DCM (700 mL). The collected filtrate was concentrated under reduced pressure to afford tert-butyl (3S)-3-(((3-(2,6-dioxopiperidin-3-yl)-1-methyl-1H-indazol-7-yl)amino)methyl) pyrrolidine-1-carboxylate (0.30 g) as a brown solid which was used in the next step without further purification.

LC-MS (ESI): m/z=464.50 [M+Na]⁺.

### Preparation of 3-(1-methyl-7-((((R)-pyrrolidin-3-yl)methyl) amino)-1H-indazol-3-yl)piperidine-2,6-dione

To a stirred solution of tert-butyl (3S)-3-(((3-(2,6-dioxopiperidin-3-yl)-1-methyl-1H-indazol-7-yl)amino)methyl) pyrrolidine-1-carboxylate (0.5 g, 1.13 mmol) in DCM (10 mL) was added 4.0 M HCl in dioxane (5 mL). The reaction mixture was stirred at room temperature for 3 h. The reaction mixture was concentrated under reduced pressure to afford 3-(1-methyl-7-((((R)-pyrrolidin-3-yl)methyl) amino)-1H-indazol-3-yl)piperidine-2,6-dione (HCl salt) (0.18 g) as a white solid which was used in the next step without purification.

LC-MS (ESI): m/z=342.40 [M+H]⁺.

### Preparation of 2-((6-((5-chloro-2-((3S)-3-(((3-(2,6-dioxopiperidin-3-yl)-1-methyl-1H-indazol-7-yl)amino)methyl) pyrrolidin-1-yl)pyrimidin-4-yl)amino)-1-methyl-2-oxo-1,2-dihydro-1,8-naphthyridin-3-yl)oxy)-N-methylacetamide

To a stirred solution of 3-(1-methyl-7-((((R)-pyrrolidin-3-yl)methyl) amino)-1H-indazol-3-yl)piperidine-2,6-dione (0.15 g, 0.39 mmol) and 2-((6-((2,5-dichloropyrimidin-4-yl)amino)-1-methyl-2-oxo-1,2-dihydro-1,8-naphthyridin-3-yl)oxy)-N-methylacetamide (0.07 g, 0.16 mmol) in DMSO (3.0 mL) was added DIPEA (0.34 mL, 1.98 mmol). The reaction mixture was stirred and heated to 100° C. for 4 h. The reaction mixture was concentrated under reduced pressure and purified by prep-HPLC to afford 2-((6-((5-chloro-2-((3S)-3-(((3-(2,6-dioxopiperidin-3-yl)-1-methyl-1H-indazol-7-yl)amino)methyl) pyrrolidin-1-yl)pyrimidin-4-yl)amino)-1-methyl-2-oxo-1,2-dihydro-1,8-naphthyridin-3-yl)oxy)-N-methylacetamide (0.029 g) as a brown solid.

¹H NMR (400 MHz, DMSO-d₆): δ 10.87 (s, 1H), 8.98 (s, 1H), 8.96-8.89 (m, 1H), 8.45-8.35 (m, 1H), 8.08 (s, 1H), 7.92 (br s, 1H), 7.21 (s, 1H), 6.97 (d, J=8.00 Hz, 1H), 6.88-6.87 (m, 1H), 6.51 (d, J=6.80 Hz, 1H), 5.45 (br s, 1H), 4.59 (br s, 2H), 4.24-4.20 (m, 4H), 3.71 (s, 3H), 3.63-3.57 (m, 2H), 3.16-3.14 (m, 2H), 2.72 (d, J=7.20 Hz, 3H), 2.66 (d, J=4.40 Hz, 3H), 2.29-2.28 (m, 1H), 2.15-2.13 (m, 2H), 1.82-1.81 (m, 1H), 1.24-1.21 (m, 2H).

LC-MS (ESI): m/z=714.34 [M+H]⁺

### Example 24:2-((6-((5-chloro-2-(4-((1-(3-(2,6-dioxopiperidin-3-yl)-1-methyl-1H-indazol-6-yl)piperidin-4-yl)methyl)-1,4-diazepan-1-yl)pyrimidin-4-yl)amino)-1-methyl-2-oxo-1,2-dihydro-1,8-naphthyridin-3-yl)oxy)-N-methylacetamide (Compound 111)

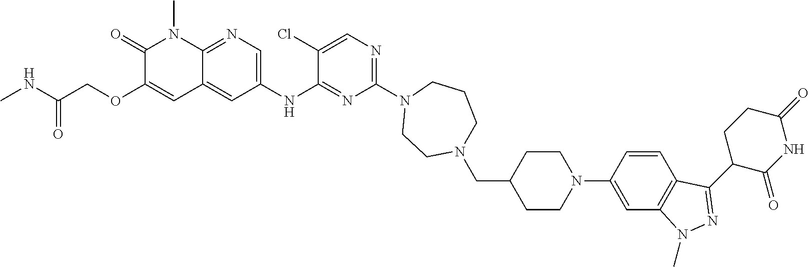 [SMILES: CNC(=O)COc1cc2cc(Nc3nc(N4CCCN(CC5CCN(c6ccc7c(C8CCC(=O)NC8=O)nn(C)c7c6)CC5)CC4)ncc3Cl)cnc2n(C)c1=O]

### Preparation of (1-(3-(2,6-bis(benzyloxy)pyridin-3-yl)-1-methyl-1H-indazol-6-yl)piperidin-4-yl)methanol

To stirred solution of 3-(2,6-bis(benzyloxy)pyridin-3-yl)-6-bromo-1-methyl-1H-indazole (1.5 g, 2.99 mmol) and piperidin-4-ylmethanol (1.38 g, 1.99 mmol) in dioxane (15 mL) was added cesium carbonate (2.92 g, 8.97 mmol). The reaction mixture was degassed for 15 minutes. Afterwards, Pd-PEPPSI-iHeptCl (0.14 g, 0.14 mmol) was added, and the mixture was stirred at 80° C. for 16 h. The reaction mixture was then cooled to room temperature and filtered through a celite bed. The filtrate was concentrated to provide a crude product which was purified by flash column chromatography (SiO₂, 230-400, 30% ethyl acetate in petroleum ether) to afford (1-(3-(2,6-bis(benzyloxy)pyridin-3-yl)-1-methyl-1H-indazol-6-yl)piperidin-4-yl)methanol (1.5 g) as a green semi-solid.

LC-MS (ESI): m/z=535.57 [M+H]⁺

### Preparation of (1-(3-(2,6-bis(benzyloxy)pyridin-3-yl)-1-methyl-1H-indazol-6-yl)piperidin-4-yl)methyl methanesulfonate

To a solution of (1-(3-(2,6-bis(benzyloxy)pyridin-3-yl)-1-methyl-1H-indazol-6-yl)piperidin-4-yl)methanol (1.5 g 2.8 mmol) in DCM (15 mL) was added triethyl amine (1.56 mL, 11.23 mmol) and methanesulfonyl chloride (0.26 mL, 3.36 mmol) at 0° C. The reaction mixture was then stirred at room temperature for 1 h. The reaction mixture was quenched with saturated sodium bicarbonate and extracted with EtOAc (3×5 mL). The combined organic layers were dried with sodium sulfate, filtered, and concentrated under vacuum to afford (1-(3-(2,6-bis(benzyloxy)pyridin-3-yl)-1-methyl-1H-indazol-6-yl)piperidin-4-yl) methyl methanesulfonate (1.50 g) as a yellow semi-solid which was used in the next step without purification.

LC-MS (ESI): m/z=613.62 [M+H]⁺.

### Preparation of tert-butyl 4-((1-(3-(2,6-bis(benzyloxy)pyridin-3-yl)-1-methyl-1H-indazol-6-yl)piperidin-4-yl)methyl)-1,4-diazepane-1-carboxylate

To a solution of (1-(3-(2,6-bis(benzyloxy)pyridin-3-yl)-1-methyl-1H-indazol-6-yl)piperidin-4-yl) methyl methanesulfonate (1.50 g, 2.45 mmol) and tert-butyl 1,4-diazepane-1-carboxylate (0.39 g, 7.35 mmol) in DMSO (15 mL) was added N,N-diisopropylethylamine (4.2 mL, 24.5 mmol). The reaction mixture was stirred at 100° C. for 2 h. Upon completion, cold water was added to the reaction mixture, and the resulting mixture was stirred for 5 min. The resulting solid precipitate was filtered under vacuum and purified by flash column (SiO₂, 100-200, 10% MeOH in DCM) to afford tert-butyl 4-((1-(3-(2,6-bis(benzyloxy)pyridin-3-yl)-1-methyl-1H-indazol-6-yl)piperidin-4-yl)methyl)-1,4-diazepane-1-carboxylate (0.8 g). LC-MS (ESI): m/z=717.83 [M+H]⁺.

### Preparation of tert-butyl 4-((1-(3-(2,6-dioxopiperidin-3-yl)-1-methyl-1H-indazol-6-yl)piperidin-4-yl)methyl)-1,4-diazepane-1-carboxylate

To a solution of tert-butyl 4-((1-(3-(2,6-bis(benzyloxy)pyridin-3-yl)-1-methyl-1H-indazol-6-yl)piperidin-4-yl)methyl)-1,4-diazepane-1-carboxylate (0.8 g, 1.11 mmol) in THF (16 mL) and acetic acid (0.4 mL) was added 20% Pd (OH)₂ on carbon, moisture 50% wet (1.25 g 8.9 mmol). The reaction mixture was stirred under hydrogen atmosphere (80 psi) at room temperature for 16 h. The reaction mixture was diluted with DCM, filtered through a celite bed, and washed with 30% THF in DCM. The collected filtrate was concentrated under vacuum to provide crude tert-butyl 4-((1-(3-(2,6-dioxopiperidin-3-yl)-1-methyl-1H-indazol-6-yl)piperidin-4-yl)methyl)-1,4-diazepane-1-carboxylate (0.49 g) as a light yellow gummy which was used in the next step without further purification.

LC-MS (ESI): m/z=539.41 [M+H]⁺.

### Preparation of 3-(6-(4-((1,4-diazepan-1-yl)methyl)piperidin-1-yl)-1-methyl-1H-indazol-3-yl)piperidine-2,6-dione

To a solution of tert-butyl 4-((1-(3-(2,6-dioxopiperidin-3-yl)-1-methyl-1H-indazol-6-yl)piperidin-4-yl)methyl)-1,4-diazepane-1-carboxylate (0.49 g,0.91 mmol) in DCM (9.8 mL) was added TFA (4.9 mL). The reaction mixture was stirred at room temperature for 2 h. The reaction mixture was then concentrated and triturated with diethyl ether to afford 3-(6-(4-((1,4-diazepan-1-yl)methyl)piperidin-1-yl)-1-methyl-1H-indazol-3-yl)piperidine-2,6-dione (0.40 g) as a TFA salt which was used in the next step without further purification. LC-MS (ESI): m/z=439.35 [M+H]⁺.

### Preparation of 2-((6-((5-chloro-2-(4-((1-(3-(2,6-dioxopiperidin-3-yl)-1-methyl-1H-indazol-6-yl)piperidin-4-yl)methyl)-1,4-diazepan-1-yl)pyrimidin-4-yl)amino)-1-methyl-2-oxo-1,2-dihydro-1,8-naphthyridin-3-yl)oxy)-N-methylacetamide

To a stirred solution of 3-(6-(4-((1,4-diazepan-1-yl)methyl)piperidin-1-yl)-1-methyl-1H-indazol-3-yl)piperidine-2,6-dione (0.20 g, 0.54 mmol) and 2-((6-((2,5-dichloropyrimidin-4-yl)amino)-1-methyl-2-oxo-1,2-dihydro-1,8-naphthyridin-3-yl)oxy)-N-methylacetamide (0.08 g, 0.218) in DMSO (2 mL) was added DIPEA (0.94 mL, 5.42 mmol). The mixture was heated to 100° C. for 6 h. The reaction mixture was quenched with ice-cold water, and the solid thus formed was filtered and purified by prep-HPLC to afford 2-((6-((5-chloro-2-(4-((1-(3-(2,6-dioxopiperidin-3-yl)-1-methyl-1H-indazol-6-yl)piperidin-4-yl)methyl)-1,4-diazepan-1-y1) pyrimidin-4-yl)amino)-1-methyl-2-oxo-1,2-dihydro-1,8-naphthyridin-3-yl)oxy)-N-methylacetamide as a pale brown solid (0.028 g).

LC-MS (ESI): m/z=811.47 [M+H]⁺.

¹H NMR (400 MHz, DMSO-d₆): δ 10.84 (s, 1H), 9.02 (s, 1H), 8.74 (s, 1H), 8.48 (br s, 1H), 8.39 (br s, 1H), 8.08 (s, 1H), 7.95 (s, 1H), 7.46 (d, J=8.80 Hz, 1H), 7.12 (s, 1H), 6.88 (d, J=8.80 Hz, 1H), 6.79 (s, 1H), 4.61 (s, 2H), 4.42-4.26 (m, 1H), 3.88 (s, 3H), 3.72-3.60 (br, 9H), 2.68-2.56 (m, 10H), 2.35-2.32 (m, 3H), 2.17-2.12 (m, 1H), 1.80-1.75 (m, 5H), 1.63-1.58 (m, 1H), 1.23-1.20 (s, 2H).

### Example 25: 2-((6-((5-chloro-2-((3R)-3-(((3-(2,6-dioxopiperidin-3-yl)-1-methyl-1H-indazol-6-yl)amino)methyl) pyrrolidin-1-yl)pyrimidin-4-yl)amino)-1-methyl-2-oxo-1,2-dihydro-1,8-naphthyridin-3-yl)oxy)-N-methylacetamide (Compound 123a)

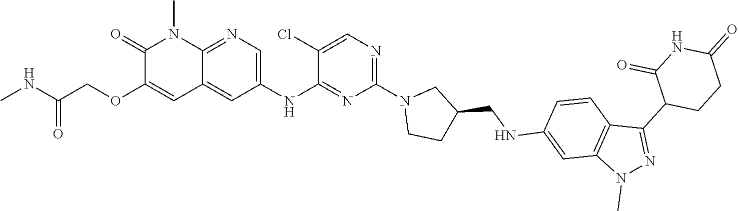 [SMILES: CNC(=O)COc1cc2cc(Nc3nc(N4CC[C@H](CNc5ccc6c(C7CCC(=O)NC7=O)nn(C)c6c5)C4)ncc3Cl)cnc2n(C)c1=O]

### Preparation of tert-butyl (R)-3-(((3-(2,6-bis(benzyloxy)pyridin-3-yl)-1-methyl-1H-indazol-6-yl)amino)methyl) pyrrolidine-1-carboxylate

To a stirred solution of tert-butyl (R)-3-(aminomethyl) pyrrolidine-1-carboxylate (0.5 g, 2.4 mmol) and 3-(2,6-bis(benzyloxy)pyridin-3-yl)-6-bromo-1-methyl-1H-indazole (0.87 g, 1.7 mmol) in 1,4-dioxane (10 mL) was added potassium tert-butoxide (0.33 g, 2.99 mmol). The reaction mixture was degassed under N₂ for 10 min. t-BrettPhos-Pd-G3 (0.10 g, 0.12 mmol) was then added, and the resulting reaction mixture was stirred at room temperature for 1 h. After completion, the reaction mixture was filtered through a celite bed and washed with ethyl acetate (100 mL). The combined organic layer was concentrated under reduced pressure and purified by flash column chromatography (SiO₂, 60-120 mesh, 15% ethyl acetate in petroleum ether) to afford tert-butyl (R)-3-(((3-(2,6-bis(benzyloxy)pyridin-3-yl)-1-methyl-1H-indazol-6-yl)amino)methyl) pyrrolidine-1-carboxylate (0.7 g).

LC-MS (ESI): m/z=620.66 [M+H]⁺.

### Preparation of tert-butyl (3R)-3-(((3-(2,6-dioxopiperidin-3-yl)-1-methyl-1H-indazol-6-yl)amino)methyl) pyrrolidine-1-carboxylate

A stirred solution of tert-butyl (R)-3-(((3-(2,6-bis(benzyloxy)pyridin-3-yl)-1-methyl-1H-indazol-6-yl)amino)methyl) pyrrolidine-1-carboxylate (0.7 g, 1.13 mmol) in THF (20 mL) was degassed with nitrogen for 5 minutes. 20% Palladium hydroxide on carbon (0.7 g, 100% w/w) was then added, and the resulting reaction mixture was stirred under hydrogen atmosphere (80 psi) at room temperature for 16 h. The reaction mixture was diluted with ethyl acetate (50 mL) and filtered through a celite bed. The filtrate was collected, concentrated under reduced pressure, and purified by trituration with diethylether to afford tert-butyl (3R)-3-(((3-(2,6-dioxopiperidin-3-yl)-1-methyl-1H-indazol-6-yl)amino)methyl) pyrrolidine-1-carboxylate (0.45 g).

LC-MS (ESI): m/z=464.64 [M+23]⁺

### Preparation of 3-(1-methyl-6-((((S)-pyrrolidin-3-yl)methyl) amino)-1H-indazol-3-yl)piperidine-2,6-dione

To a stirred solution of tert-butyl (3R)-3-(((3-(2,6-dioxopiperidin-3-yl)-1-methyl-1H-indazol-6-yl)amino)methyl) pyrrolidine-1-carboxylate (0.45 g, 1.01 mmol) in DCM (4.5 mL) was added HCl in dioxane (4 M, 2.25 mL) at 0° C. under N₂ atmosphere. The resulting reaction mixture was stirred at room temperature for 3 h. The reaction mixture was concentrated under reduced pressure and co-distilled with DCM. The crude product was purified by trituration with n-pentane to afford 3-(1-methyl-6-((((S)-pyrrolidin-3-yl)methyl) amino)-1H-indazol-3-yl)piperidine-2,6-dione (0.25 g)

LC-MS (ESI): m/z=342.44 [M+H]⁺

### Preparation of 2-((6-((5-chloro-2-((3R)-3-(((3-(2,6-dioxopiperidin-3-yl)-1-methyl-1H-indazol-6-yl)amino)methyl) pyrrolidin-1-yl)pyrimidin-4-yl)amino)-1-methyl-2-oxo-1,2-dihydro-1,8-naphthyridin-3-yl)oxy)-N-methylacetamide

To a stirred solution of 3-(1-methyl-6-((((S)-pyrrolidin-3-yl)methyl) amino)-1H-indazol-3-yl)piperidine-2,6-dione (0.1 g, 0.26 mmol) in DMSO (2.0 mL) was added DIPEA (0.23 mL, 1.3 mmol). The reaction mixture was stirred at room temperature for 5 min under N₂ atmosphere. 2-((6-((2,5-dichloropyrimidin-4-yl)amino)-1-methyl-2-oxo-1,2-dihydro-1,8-naphthyridin-3-yl)oxy)-N-methylacetamide (0.04 g, 0.1 mmol) was then added, and the resulting reaction mixture was stirred and heated to 100° C. for 3 h. The reaction mixture was concentrated under reduced pressure and purified by prep-HPLC to afford 2-((6-((5-chloro-2-((3R)-3-(((3-(2,6-dioxopiperidin-3-yl)-1-methyl-1H-indazol-6-yl)amino)methyl) pyrrolidin-1-yl)pyrimidin-4-yl)amino)-1-methyl-2-oxo-1,2-dihydro-1,8-naphthyridin-3-yl)oxy)-N-methylacetamide (12 mg) as a brown solid.

LC-MS (ESI): m/z=714.46 [M+H]⁺

¹H NMR (400 MHz, DMSO-d₆): δ 10.81 (s, 1H), 8.98 (s, 1H), 8.83 (s, 1H), 8.43 (br, 3H), 8.07 (s, 1H), 7.94 (s, 1H), 7.33-7.30 (m, 1H), 7.20 (s, 1H), 6.56-6.54 (m, 1H), 6.07-6.06 (m, 1H), 4.60 (s, 2H), 4.18-4.15 (m, 1H), 3.78-3.72 (m, 7H), 3.56-3.54 (m, 1H), 3.14 (m, 4H), 2.65-2.64 (m, 6H), 2.23-2.24 (m, 1H), 1.81-1.76 (m, 1H), 1.23 (s, 1H).

### Example 26:2-((6-((2-(4-((1-(3-(2,6-dioxopiperidin-3-yl)-1-methyl-1H-indazol-6-yl)piperidin-4-yl)methyl) piperazin-1-yl)-5-fluoropyrimidin-4-yl)amino)-1-methyl-2-oxo-1,2-dihydro-1,8-naphthyridin-3-yl)oxy)-N-methylacetamide (Compound 134)

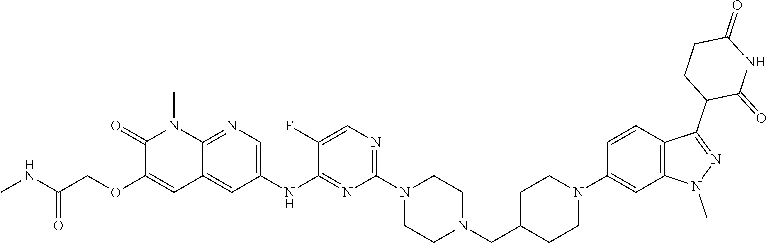 [SMILES: CNC(=O)COc1cc2cc(Nc3nc(N4CCN(CC5CCN(c6ccc7c(C8CCC(=O)NC8=O)nn(C)c7c6)CC5)CC4)ncc3F)cnc2n(C)c1=O]

### Preparation of 2-((6-((2-chloro-5-fluoropyrimidin-4-yl)amino)-1-methyl-2-oxo-1,2-dihydro-1,8-naphthyridin-3-yl)oxy)-N-methylacetamide

To a stirred solution of 2-((6-amino-1-methyl-2-oxo-1,2-dihydro-1,8-naphthyridin-3-yl)oxy)-N-methylacetamide (0.50 g, 1.90 mmol) and 2,4-dichloro-5-fluoropyrimidine (0.70 g, 4.19 mmol) in DMSO (10.0 mL) was added N,N-diisopropylethylamine (0.79 mL, 5.71 mmol). The resulting reaction mixture was stirred at 50° C. for 5 h. The reaction mixture was quenched with water (10.0 mL) and extracted with ethyl acetate (3×20.0 mL). The combined organic layers were washed with brine (20.0 mL), dried over anhydrous Na₂SO₄, filtered, and concentrated under reduced pressure to provide a crude product which was purified by column chromatography 100-200 silica gel (product eluted in 5% MeOH in DCM) to provide 2-((6-((2-chloro-5-fluoropyrimidin-4-yl)amino)-1-methyl-2-oxo-1,2-dihydro-1,8-naphthyridin-3-yl)oxy)-N-methylacetamide as brown solid. (0.34 g)

LC-MS (ESI): m/z=393.11 [M+H]⁺.

### Preparation of 2-((6-((2-(4-((1-(3-(2,6-dioxopiperidin-3-yl)-1-methyl-1H-indazol-6-yl)piperidin-4-yl)methyl) piperazin-1-yl)-5-fluoropyrimidin-4-yl)amino)-1-methyl-2-oxo-1,2-dihydro-1,8-naphthyridin-3-yl)oxy)-N-methylacetamide

To a stirred solution of 3-(1-methyl-6-(4-(piperazin-1-ylmethyl)piperidin-1-yl)-1H-indazol-3-yl)piperidine-2,6-dione hydrochloride (0.15 g, 0.32 mmol) and 2-((6-((2-chloro-5-fluoropyrimidin-4-yl)amino)-1-methyl-2-oxo-1,2-dihydro-1,8-naphthyridin-3-yl)oxy)-N-methylacetamide (0.05 g, 0.13 mmol) in DMSO (3 mL) was added N,N-diisopropylethylamine (0.28 ml, 1.62 mmol) at room temperature. The reaction mixture was then stirred at 100° C. for 4 h. After completion, the reaction mixture was concentrated under reduced pressure and purified by prep-HPLC to afford 2-((6-((2-(4-((1-(3-(2,6-dioxopiperidin-3-yl)-1-methyl-1H-indazol-6-yl)piperidin-4-yl)methyl) piperazin-1-yl)-5-fluoropyrimidin-4-yl)amino)-1-methyl-2-oxo-1,2-dihydro-1,8-naphthyridin-3-yl)oxy)-N-methylacetamide as a pink solid (12 mg).

LC-MS (ESI): m/z=781.55 [M+H]⁺.

¹H NMR: 400 MHz, DMSO: δ 10.84 (s, 1H), 9.60 (s, 1H), 8.78 (d, J=2.00 Hz, 1H), 8.38 (d, J=2.40 Hz, 1H), 8.07 (d, J=3.60 Hz, 1H), 7.95 (d, J=4.40 Hz, 1H), 7.47 (d, J=9.20 Hz, 1H), 7.18 (s, 1H), 6.90 (d, J=8.80 Hz, 1H), 6.82 (s, 1H), 4.63 (s, 2H), 4.23-4.24 (m, 1H), 3.88 (s, 3H), 3.80-3.77 (m, 2H), 3.73 (s, 3H), 3.63 (bs, 4H), 2.76-2.60 (m, 7H), 2.42 (m, 4H), 2.22-2.20 (m, 4H), 1.82-1.77 (m, 3H), 1.28-1.25 (m, 2H).

### Example 27: 2-((6-((2-(4-((1-(3-(2,6-dioxopiperidin-3-yl)-1-methyl-1H-indazol-7-yl)piperidin-4-yl)methyl) piperazin-1-yl)-5-fluoropyrimidin-4-yl)amino)-1-methyl-2-oxo-1,2-dihydro-1,8-naphthyridin-3-yl)oxy)-N-methylacetamide (Compound 135)

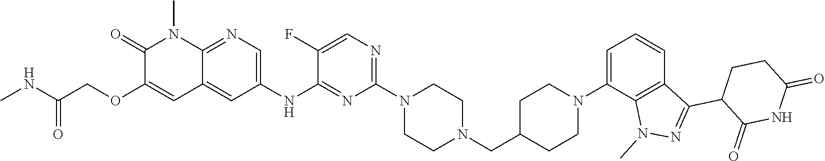 [SMILES: CNC(=O)COc1cc2cc(Nc3nc(N4CCN(CC5CCN(c6cccc7c(C8CCC(=O)NC8=O)nn(C)c67)CC5)CC4)ncc3F)cnc2n(C)c1=O]

### Preparation of 2-((6-((2-(4-((1-(3-(2,6-dioxopiperidin-3-yl)-1-methyl-1H-indazol-7-yl)piperidin-4-yl)methyl) piperazin-1-yl)-5-fluoropyrimidin-4-yl)amino)-1-methyl-2-oxo-1,2-dihydro-1,8-naphthyridin-3-yl)oxy)-N-methylacetamide

To a stirred solution of 3-(1-methyl-7-(4-(piperazin-1-ylmethyl)piperidin-1-yl)-1H-indazol-3-yl)piperidine-2,6-dione (0.15 g, 0.32 mmol) and 2-((6-((2-chloro-5-fluoropyrimidin-4-yl)amino)-1-methyl-2-oxo-1,2-dihydro-1,8-naphthyridin-3-yl)oxy)-N-methylacetamide (0.05 g, 0.13 mmol) in DMSO (3 mL) was added N,N-diisopropylethylamine (0.28 ml, 1.62 mmol) at room temperature. The resulting mixture was stirred at 100° C. for 4 h. After completion, the resulting reaction mixture was concentrated under reduced pressure and purified by prep-HPLC to provide 2-((6-((2-(4-((1-(3-(2,6-dioxopiperidin-3-yl)-1-methyl-1H-indazol-7-yl)piperidin-4-yl)methyl) piperazin-1-yl)-5-fluoropyrimidin-4-yl)amino)-1-methyl-2-oxo-1,2-dihydro-1,8-naphthyridin-3-yl)oxy)-N-methylacetamide as a pink solid (23 mg).

LC-MS (ESI): m/z=781.60 [M+H]⁺.

1HNMR: 400 MHz, DMSO: δ 10.84 (s, 1H), 9.60 (s, 1H), 8.78 (s, 1H), 8.39-8.38 (m, 3H), 8.07 (d, J=3.60 Hz, 1H), 7.95 (d, J=4.40 Hz, 1H), 7.47 (d, J=9.20 Hz, 1H), 7.18 (s, 1H), 6.90 (d, J=8.80 Hz, 1H), 4.63 (s, 2H), 4.23-4.24 (m, 1H), 3.88 (m, 3H), 3.80 (m, 2H), 3.77-3.73 (m, 3H), 3.63 (s, 4H), 2.62-2.61 (m, 6H), 2.42 (m, 4H), 2.22-2.20 (m, 4H), 1.82-1.77 (m, 3H), 1.28-1.25 (m, 2H).

### Example 28:2-((6-((5-chloro-2-(9-((1-(3-(2,6-dioxopiperidin-3-yl)-1-methyl-1H-indazol-6-yl)piperidin-4-yl)methyl)-3,9-diazaspiro[5.5]undecan-3-yl)pyrimidin-4-yl)amino)-1-methyl-2-oxo-1,2-dihydro-1,8-naphthyridin-3-yl)oxy)-N-methylacetamide (Compound

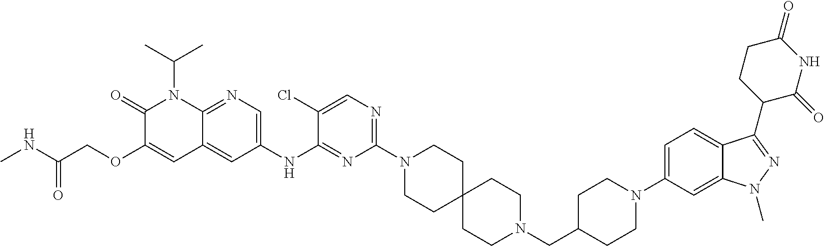 [SMILES: CNC(=O)COc1cc2cc(Nc3nc(N4CCC5(CCN(CC6CCN(c7ccc8c(C9CCC(=O)NC9=O)nn(C)c8c7)CC6)CC5)CC4)ncc3Cl)cnc2n(C(C)C)c1=O]

### Preparation of tert-butyl 9-((1-(3-(2,6-bis(benzyloxy)pyridin-3-yl)-1-methyl-1H-indazol-6-yl)piperidin-4-yl)methyl)-3,9-diazaspiro[5.5]undecane-3-carboxylate

To a stirred solution of 3-(2,6-bis(benzyloxy)pyridin-3-yl)-6-bromo-1-methyl-1H-indazole (1.0 g, 2.0 mmol) and tert-butyl 9-(piperidin-4-ylmethyl)-3,9-diazaspiro[5.5]undecane-3-carboxylate (1.40 g, 4.0 mmol, prepared e.g., using published methods in e.g., International Application Publication No. WO2022228547) in 1,4-dioxane (20 mL) was added cesium carbonate (1.95 g, 6.0 mmol). The reaction was degassed with argon for 10 minutes. RuPhos (0.09 g, 0.20 mmol) and RuPhos-PdG3 (0.08 g, 0.10 mmol) were added, and the reaction was degassed for 5 min. The reaction mixture was stirred at 80° C. for 3 h. The reaction mixture was cooled to room temperature and filtered through a celite bed. The filtrate was concentrated to provide a crude product. The crude product was purified by flash column chromatography (SiO₂, 230-400, 30% ethyl acetate in petroleum ether) to afford tert-butyl 9-((1-(3-(2,6-bis(benzyloxy)pyridin-3-yl)-1-methyl-1H-indazol-6-yl)piperidin-4-yl)methyl)-3,9-diazaspiro[5.5]undecane-3-carboxylate (0.7 g) as an off-white solid.

LC-MS (ESI): m/z=771.83 [M+H]⁺.

### Preparation of tert-butyl 9-((1-(3-(2,6-dioxopiperidin-3-yl)-1-methyl-1H-indazol-6-yl)piperidin-4-yl)methyl)-3,9-diazaspiro[5.5]undecane-3-carboxylate

To a stirred solution of tert-butyl 9-((1-(3-(2,6-bis(benzyloxy)pyridin-3-yl)-1-methyl-1H-indazol-6-yl)piperidin-4-yl)methyl)-3,9-diazaspiro[5.5]undecane-3-carboxylate (0.7 g, 0.90 mmol) in THF (28 mL) was added 20% Pd (OH)₂ (0.7 g) at room temperature. The reaction was put under hydrogen pressure (60 Psi) and stirred for 8 h at room temperature. The reaction mixture was filtered through a celite bed. The celite was washed with ethyl acetate and concentrated under reduced pressure to provide a crude product. The crude product was washed with n-pentane and dried to afford tert-butyl 9-((1-(3-(2,6-dioxopiperidin-3-yl)-1-methyl-1H-indazol-6-yl)piperidin-4-yl)methyl)-3,9-diazaspiro[5.5]undecane-3-carboxylate (0.60 g) as a brown solid.

LC-MS (ESI): m/z=593.67 [M+H]⁺.

### Preparation of 3-(6-(4-((3,9-diazaspiro[5.5]undecan-3-yl)methyl)piperidin-1-yl)-1-methyl-1H-indazol-3-yl)piperidine-2,6-dione

A stirred solution of tert-butyl 9-((1-(3-(2,6-dioxopiperidin-3-yl)-1-methyl-1H-indazol-6-yl)piperidin-4-yl)methyl)-3,9-diazaspiro[5.5]undecane-3-carboxylate (0.6 g, 1.01 mmol) in DCM (12 mL) was cooled to 0° C. TFA (6 mL) was added, and the reaction mixture was allowed to warm to room temperature over 3 h with stirring. The reaction mixture was concentrated under reduced pressure to provide a crude product. The crude product was triturated with diethyl ether (20 mL) to afford 3-(6-(4-((3,9-diazaspiro[5.5]undecan-3-yl)methyl)piperidin-1-yl)-1-methyl-1H-indazol-3-yl)piperidine-2,6-dione (0.44 g) as a brown solid.

LC-MS (ESI): m/z=493.37 [M+H]⁺.

### Preparation of 2-((6-((5-chloro-2-(9-((1-(3-(2,6-dioxopiperidin-3-yl)-1-methyl-1H-indazol-6-yl)piperidin-4-yl)methyl)-3,9-diazaspiro[5.5]undecan-3-yl)pyrimidin-4-yl)amino)-1-methyl-2-oxo-1,2-dihydro-1,8-naphthyridin-3-yl)oxy)-N-methylacetamide

To a stirred solution of 3-(6-(4-((3,9-diazaspiro[5.5]undecan-3-yl)methyl)piperidin-1-yl)-1-methyl-1H-indazol-3-yl)piperidine-2,6-dione (0.22 g, 0.44 mmol) and 2-((6-((2,5-dichloropyrimidin-4-yl)amino)-1-methyl-2-oxo-1,2-dihydro-1,8-naphthyridin-3-yl)oxy)-N-methylacetamide (0.07 g, 0.17 mmol) in DMSO (4 mL) was added N,N-diisopropylethylamine (0.8 mL, 3.57 mmol). The reaction mixture was stirred at 100° C. for 4 h. Solvent was removed to provide a crude product which was purified by prep-HPLC to afford 2-((6-((5-chloro-2-(9-((1-(3-(2,6-dioxopiperidin-3-yl)-1-methyl-1H-indazol-6-yl)piperidin-4-yl)methyl)-3,9-diazaspiro[5.5]undecan-3-yl)pyrimidin-4-yl)amino)-1-methyl-2-oxo-1,2-dihydro-1,8-naphthyridin-3-yl)oxy)-N-methylacetamide (50 mg) as an off white solid.

¹H NMR: 400 MHz, DMSO-d₆: δ 10.85 (s, 1H), 9.03 (s, 1H), 8.72 (d, J=2.4 Hz, 1H), 8.30-8.28 (m, 1H), 8.06 (s, 1H), 8.00-7.94 (m, 1H), 7.46 (d, J=9.2 Hz, 1H), 7.15 (s, 1H), 6.89 (d, J=9.2 Hz, 1H), 6.81 (s, 1H), 4.621 (s, 2H), 4.26-4.22 (m, 1H), 3.87 (s, 3H), 3.77-3.73 (m, 4H), 3.60 (s, 4H), 2.80-2.70 (m, 7H), 2.40-2.22 (m, 3H), 1.50-1.30 (m, 8H), 1.30-1.20 (m, 2H). LC-MS (ESI): m/z=865.44 [M+H]⁺

### Example 29:2-((6-((5-chloro-2-(9-((1-(3-(2,6-dioxopiperidin-3-yl)-1-methyl-1H-indazol-7-yl)piperidin-4-yl)methyl)-3-oxa-7,9-diazabicyclo[3.3.1]nonan-7-yl)pyrimidin-4-yl)amino)-1-methyl-2-oxo-1,2-dihydro-1,8-naphthyridin-3-yl)oxy)-N-methylacetamide (Compound 117)

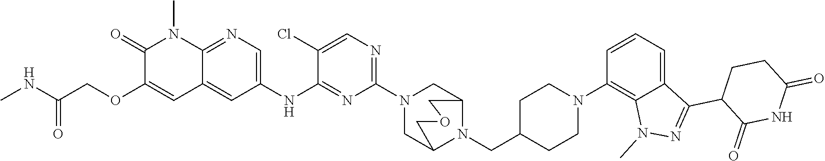 [SMILES: CNC(=O)COc1cc2cc(Nc3nc(N4CC5COCC(C4)N5CC4CCN(c5cccc6c(C7CCC(=O)NC7=O)nn(C)c56)CC4)ncc3Cl)cnc2n(C)c1=O]

### Preparation of tert-butyl 9-((1-((benzyloxy) carbonyl)piperidin-4-yl)methyl)-3-oxa-7,9-diazabicyclo[3.3.1]nonane-7-carboxylate

To a solution of tert-butyl 3-oxa-7,9-diazabicyclo[3.3.1]nonane-7-carboxylate (1.0 g, 4.38 mmol) and benzyl 4-formylpiperidine-1-carboxylate (0.97 g, 3.94 mmol) in DCM (20.0 mL, 20 V) was added acetic acid (0.25 mL, 4.38 mmol). The reaction mixture was stirred for 1 h. Sodium triacetoxyborohydride (1.85 g, 8.76 mmol) was then added at 0° C., and the resultant reaction mixture was stirred at room temperature for 16 h. The reaction mixture was then quenched with NH₄Cl (30 mL) and extracted with DCM (2×40 mL). The combined organic layers were washed with brine (20 mL), dried over anhydrous Na₂SO₄, filtered, and concentrated under reduced pressure to afford tert-butyl 9-((1-((benzyloxy) carbonyl)piperidin-4-yl)methyl)-3-oxa-7,9-diazabicyclo[3.3.1]nonane-7-carboxylate (1.35 g) as a pale yellow semi-solid which was used in the next step without further purification.

LC-MS (ESI): m/z=460.60 [M+H]⁺

### Preparation of tert-butyl 9-(piperidin-4-ylmethyl)-3-oxa-7,9-diazabicyclo[3.3.1]nonane-7-carboxylate

To a solution of tert-butyl 9-((1-((benzyloxy) carbonyl)piperidin-4-yl)methyl)-3-oxa-7,9-diazabicyclo[3.3.1]nonane-7-carboxylate (1.30 g, 2.82 mmol) in THF (78.0 mL, 60 V) was added 20% Pd (OH)₂/C (0.65 g, 50% W/W). The reaction mixture was stirred under hydrogen atmosphere (80 psi) at room temperature for 5 h. The reaction mixture was diluted with THF (50 mL), filtered through a celite bed and washed with 200 mL of THF: DCM (1:1). The filtrate was concentrated and dried to afford tert-butyl 9-(piperidin-4-ylmethyl)-3-oxa-7,9-diazabicyclo[3.3.1]nonane-7-carboxylate as a black semi-solid (0.97 g) which was used in the next step without further purification.

LC-MS (ESI): m/z=326.24 [M+H]⁺

### Preparation of tert-butyl 9-((1-(3-(2,6-bis(benzyloxy)pyridin-3-yl)-1-methyl-1H-indazol-7-yl)piperidin-4-yl)methyl)-3-oxa-7,9-diazabicyclo[3.3.1]nonane-7-carboxylate

To a stirred solution of 3-(2,6-bis(benzyloxy)pyridin-3-yl)-7-bromo-1-methyl-1H-indazole (1.0 g, 2.00 mmol) and tert-butyl 9-(piperidin-4-ylmethyl)-3-oxa-7,9-diazabicyclo[3.3.1]nonane-7-carboxylate (0.97 g, 3.00 mmol) in 1,4-dioxane (706.0 mL) was added cesium carbonate (1.95 g, 6.00 mmol). The reaction mixture was degassed with argon for 15 minutes. Pd-PEPPSI-IHeptCl (0.10 g, 0.10 mmol) was then added, and the reaction mixture was purged again for 2 minutes. The resulting reaction mixture was stirred at 100° C. for 16 h. The reaction mixture was cooled to room temperature, diluted with ethyl acetate (40 mL), filtered through a celite bed, and washed with 10% MeOH in DCM (100 mL). The filtrate was concentrated and purified by flash chromatography (230-400 silica gel; 35% ethyl acetate in petroleum ether) to afford product tert-butyl 9-((1-(3-(2,6-bis(benzyloxy)pyridin-3-yl)-1-methyl-1H-indazol-7-yl)piperidin-4-yl)methyl)-3-oxa-7,9-diazabicyclo[3.3.1]nonane-7-carboxylate as a pale yellow solid (0.48 g) as a pale yellow solid.

LC-MS (ESI): m/z=745.87 [M+H]⁺

### Preparation of tert-butyl 9-((1-(3-(2,6-dioxopiperidin-3-yl)-1-methyl-1H-indazol-7-yl)piperidin-4-yl)methyl)-3-oxa-7,9-diazabicyclo[3.3.1]nonane-7-carboxylate

To a solution of tert-butyl 9-((1-(3-(2,6-bis(benzyloxy)pyridin-3-yl)-1-methyl-1H-indazol-7-yl)piperidin-4-yl)methyl)-3-oxa-7,9-diazabicyclo[3.3.1]nonane-7-carboxylate (0.48 g, 0.64 mmol) in THF (24 mL) was added 20% Pd (OH)₂/C (0.50 g, 100% w/w). The reaction mixture was stirred under hydrogen atmosphere (80 psi) at room temperature for 16 h. The reaction mixture was then diluted with THF (40 mL), filtered through a celite bed, and washed with THF: DCM (1:1, 100 mL). The filtrate was concentrated and dried to obtain tert-butyl 9-((1-(3-(2,6-dioxopiperidin-3-yl)-1-methyl-1H-indazol-7-yl)piperidin-4-yl)methyl)-3-oxa-7,9-diazabicyclo[3.3.1]nonane-7-carboxylate (0.50 g) as a black solid which was used in the next step without further purification.

LC-MS (ESI): m/z=567.68 [M+H]⁺.

### Preparation of 3-(7-(4-((3-oxa-7,9-diazabicyclo[3.3.1]nonan-9-yl)methyl)piperidin-1-yl)-1-methyl-1H-indazol-3-yl)piperidine-2,6-dione

To a solution of tert-butyl 9-((1-(3-(2,6-dioxopiperidin-3-yl)-1-methyl-1H-indazol-7-yl)piperidin-4-yl)methyl)-3-oxa-7,9-diazabicyclo[3.3.1]nonane-7-carboxylate (0.50 g, 0.88 mmol) in DCM (10.0 mL) was added TFA (2.5 mL) at 0° C. and stirred at room temperature for 3 h. The reaction mixture was concentrated under reduced pressure, co-distilled with DCM (2×10 mL), and triturated with diethyl ether (2×15 mL) to afford 3-(7-(4-((3-oxa-7,9-diazabicyclo[3.3.1]nonan-9-yl)methyl)piperidin-1-yl)-1-methyl-1H-indazol-3-yl)piperidine-2,6-dione as a pale yellow semi-solid (0.35 g).

LC-MS (ESI): m/z=467.32 [M+H]⁺.

### Preparation of 2-((6-((5-chloro-2-(9-((1-(3-(2,6-dioxopiperidin-3-yl)-1-methyl-1H-indazol-7-yl)piperidin-4-yl)methyl)-3-oxa-7,9-diazabicyclo[3.3.1]nonan-7-yl)pyrimidin-4-yl)amino)-1-methyl-2-oxo-1,2-dihydro-1,8-naphthyridin-3-yl)oxy)-N-methylacetamide

To a solution of 3-(7-(4-((3-oxa-7,9-diazabicyclo[3.3.1]nonan-9-yl)methyl)piperidin-1-yl)-1-methyl-1H-indazol-3-yl)piperidine-2,6-dione (0.18 g, 0.38 mmol) in DMSO (1.8 mL, 10 V) was added DIPEA (1.8 mL) and 2-((6-((2,5-dichloropyrimidin-4-yl)amino)-1-methyl-2-oxo-1,2-dihydro-1,8-naphthyridin-3-yl)oxy)-N-methylacetamide (0.063 g, 0.15 mmol) and stirred at 100° C. for 16 h. The reaction mixture was concentrated under reduced pressure and purified by prep-HPLC to afford 2-((6-((5-chloro-2-(9-((1-(3-(2,6-dioxopiperidin-3-yl)-1-methyl-1H-indazol-7-yl)piperidin-4-yl)methyl)-3-oxa-7,9-diazabicyclo[3.3.1]nonan-7-yl)pyrimidin-4-yl)amino)-1-methyl-2-oxo-1,2-dihydro-1,8-naphthyridin-3-yl)oxy)-N-methylacetamide as an off white solid (0.044 g).

¹H NMR, 400 MHz, DMSO-d6: δ 10.89 (s, 1H), 9.01 (s, 1H), 8.75 (d, J=2.40 Hz, 1H), 8.38 (d, J=2.40 Hz, 1H), 8.08 (s, 1H), 7.94 (d, J=4.40 Hz, 1H), 7.35-7.35 (m, 1H), 7.18 (s, 1H), 7.01-7.00 (m, 2H), 4.62 (s, 2H), 4.35-4.24 (m, 5H), 4.05-4.02 (m, 1H), 3.81-3.67 (m, 7H), 3.40 (d, J=12.40 Hz, 2H), 3.29-3.20 (m, 2H), 2.74-2.61 (m, 10H), 2.60-2.32 (m, 3H), 1.91 (d, J=12.00 Hz, 2H), 1.65 (br s, 1H), 1.45-1.33 (m, 2H).

LC-MS (ESI): m/z=839.59 [M+H]⁺

### Example 30: 2-((6-((5-chloro-2-((1R,5S,6s)-6-((4-(3-(2,6-dioxopiperidin-3-yl)-1-methyl-1H-indazol-7-yl)piperazin-1-yl)methyl)-3-azabicyclo[3.1.0]hexan-3-yl)pyrimidin-4-yl)amino)-1-methyl-2-oxo-1,2-dihydro-1,8-naphthyridin-3-yl)oxy)-N-methylacetamide (Compound 136a)

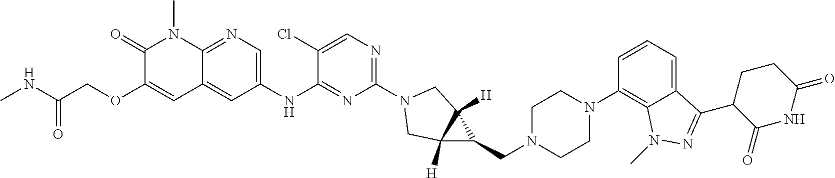 [SMILES: CNC(=O)COc1cc2cc(Nc3nc(N4C[C@@H]5[C@@H](CN6CCN(c7cccc8c(C9CCC(=O)NC9=O)nn(C)c78)CC6)[C@@H]5C4)ncc3Cl)cnc2n(C)c1=O]

### Preparation of tert-butyl (1R,5S,6s)-6-((4-((benzyloxy) carbonyl)piperazin-1-yl)methyl)-3-azabicyclo[3.1.0]hexane-3-carboxylate

To a stirred solution of tert-butyl (1R,5S,6r)-6-formyl-3-azabicyclo[3.1.0]hexane-3-carboxylate (2.0 g, 9.46 mmol) and benzyl piperazine-1-carboxylate (2.08 g, 9.44 mmol) in DCE (40 mL) was added acetic acid (0.54 mL, 9.32 mmol). The reaction mixture was stirred for 1 h. To the mixture was then added sodium triacetoxyborohydride (4.01 g, 18.92 mmol). The resulting mixture was stirred at room temperature for 16 h. The reaction mixture was quenched with ammonium chloride solution (70 mL) and extracted with DCM (2×80 mL). The combined organic layer was washed with brine (50 mL), dried over anhydrous Na₂SO₄, filtered, and concentrated under reduced pressure to afford tert-butyl (1R,5S,6s)-6-((4-((benzyloxy) carbonyl)piperazin-1-yl)methyl)-3-azabicyclo[3.1.0]hexane-3-carboxylate as a pale yellow sticky compound (3.5 g) which was used in the next step without further purification.

¹H NMR, 400 MHz, CDCl₃: δ 7.37-7.26 (m, 5H), 5.13 (s, 2H), 3.62-3.51 (m, 7H), 3.33 (t, J=9.60 Hz, 2H), 2.45 (s, 4H), 2.31 (d, J=6.80 Hz, 2H), 1.43 (s, 9H), 1.32 (s, 2H).

LC-MS (ESI): m/z=416.32 [M+H]⁺.

### Preparation of tert-butyl (1R,5S,6s)-6-(piperazin-1-ylmethyl)-3-azabicyclo[3.1.0]hexane-3-carboxylate

To a solution of tert-butyl (1R,5S,6s)-6-((4-((benzyloxy) carbonyl)piperazin-1-yl)methyl)-3-azabicyclo[3.1.0]hexane-3-carboxylate (3.50 g, 6.76 mmol) in THF (105.0 mL, 30 V) was added 20% Pd(OH)₂/C (1.75 g, 50% w/w). The reaction mixture was stirred under hydrogen atmosphere (80 psi) at room temperature for 16 h. The reaction mixture was diluted with THE (50 mL), filtered through a celite bed, and washed with THF:DCM (1:1, 200 mL). The filtrate was concentrated and dried to afford tert-butyl (1R,5S,6s)-6-(piperazin-1-ylmethyl)-3-azabicyclo[3.1.0]hexane-3-carboxylate as a pale-yellow gummy compound (2.10 g) which was used in the next step without further purification.

¹H NMR, 400 MHz, DMSO-d6: δ 3.40-3.34 (m, 2H), 3.39-3.28 (m, 2H), 2.73 (t, J=4.80 Hz, 4H), 2.49 (s, 4H), 2.20 (d, J=6.80 Hz, 2H), 1.37-1.34 (m, 12H), 0.54-0.46 (m, 1H).

### Preparation of tert-butyl (1R,5S,6s)-6-((4-(3-(2,6-bis(benzyloxy)pyridin-3-yl)-1-methyl-1H-indazol-7-yl)piperazin-1-yl)methyl)-3-azabicyclo[3.1.0]hexane-3-carboxylate

To a stirred solution of 3-(2,6-bis(benzyloxy)pyridin-3-yl)-7-bromo-1-methyl-1H-indazole (0.70 g, 1.39 mmol) and tert-butyl (1R,5S,6s)-6-(piperazin-1-ylmethyl)-3-azabicyclo[3.1.0]hexane-3-carboxylate (1.18 g, 4.19 mmol) in 1,4-dioxane (14.0 mL, 20 V) was added cesium carbonate (1.36 g, 4.19 mmol). The reaction mixture was degassed with argon for 15 minutes. Then to the mixture was added Pd-PEPPSI-IHeptCl (0.068 g, 0.07 mmol), and the resulting mixture was stirred at 100° C. for 16 h. The reaction mixture was diluted with ethyl acetate (50 mL), filtered through a celite bed, and washed with MeOH:DCM (10%, 250 mL). The filtrate was concentrated, dried under vacuum and purified by flash chromatography (230-400 silica gel; 30-35% ethyl acetate in petroleum ether) to afford tert-butyl (1R,5S,6s)-6-((4-(3-(2,6-bis(benzyloxy)pyridin-3-yl)-1-methyl-1H-indazol-7-yl)piperazin-1-yl)methyl)-3-azabicyclo[3.1.0]hexane-3-carboxylate (0.51 g) as a yellow sticky compound.

LC-MS (ESI): m/z=701.79 [M+H]⁺

### Preparation of tert-butyl (1R,5S,6s)-6-((4-(3-(2,6-dioxopiperidin-3-yl)-1-methyl-1H-indazol-7-yl)piperazin-1-yl)methyl)-3-azabicyclo[3.1.0]hexane-3-carboxylate

To a solution of tert-butyl (1R,5S,6s)-6-((4-(3-(2,6-bis(benzyloxy)pyridin-3-yl)-1-methyl-1H-indazol-7-yl)piperazin-1-yl)methyl)-3-azabicyclo[3.1.0]hexane-3-carboxylate (0.51 g, 0.72 mmol) in THF (20.0 mL) was added 20% Pd(OH)₂/C (0.51 g, 100% w/w). The reaction mixture was stirred under hydrogen atmosphere (80 psi) at room temperature for 5 h. The reaction mixture was diluted with THF (50 mL) and filtered through a celite bed and washed with THF:DCM (1:1, 150 mL). The filtrate was concentrated and dried to afford tert-butyl (1R,5S,6s)-6-((4-(3-(2,6-dioxopiperidin-3-yl)-1-methyl-1H-indazol-7-yl)piperazin-1-yl)methyl)-3-azabicyclo[3.1.0]hexane-3-carboxylate as a pale brown solid (0.41 g) which was used in the next step without further purification.

LC-MS (ESI): m/z=523.67 [M+H]⁺

### Preparation of 3-(7-(4-(((1R,5S,6r)-3-azabicyclo[3.1.0]hexan-6-yl)methyl)piperazin-1-yl)-1-methyl-1H-indazol-3-yl)piperidine-2,6-dione

To a solution of tert-butyl (1R,5S,6s)-6-((4-(3-(2,6-dioxopiperidin-3-yl)-1-methyl-1H-indazol-7-yl)piperazin-1-yl)methyl)-3-azabicyclo[3.1.0]hexane-3-carboxylate (0.41 g, 0.78 mmol) in DCM (4.0 mL) was added TFA (2.1 mL) at 0° C. The reaction mixture was stirred at room temperature for 3 h. The reaction mixture was concentrated under reduced pressure, co-distilled with DCM (3×10 mL), and dried to afford 3-(7-(4-(((1R,5S,6r)-3-azabicyclo[3.1.0]hexan-6-yl)methyl)piperazin-1-yl)-1-methyl-1H-indazol-3-yl)piperidine-2,6-dione (0.51 g) as an off white solid which was used in the next step without further purification.

LC-MS (ESI): m/z=423.28 [M+H]⁺.

### Preparation of 2-((6-((5-chloro-2-((1R,5S,6s)-6-((4-(3-(2,6-dioxopiperidin-3-yl)-1-methyl-1H-indazol-7-yl)piperazin-1-yl)methyl)-3-azabicyclo[3.1.0]hexan-3-yl)pyrimidin-4-yl)amino)-1-methyl-2-oxo-1,2-dihydro-1,8-naphthyridin-3-yl)oxy)-N-methylacetamide

To a solution of 3-(7-(4-(((1R,5S,6r)-3-azabicyclo[3.1.0]hexan-6-yl)methyl)piperazin-1-yl)-1-methyl-1H-indazol-3-yl)piperidine-2,6-dione (0.25 g, 0.59 mmol) in DMSO (2.5 mL) and DIPEA (2.0 mL) was added 2-((6-((2,5-dichloropyrimidin-4-yl)amino)-1-methyl-2-oxo-1,2-dihydro-1,8-naphthyridin-3-yl)oxy)-N-methylacetamide (0.09 g, 0.22 mmol). The reaction mixture was stirred at 100° C. for 16 h. The reaction mixture was concentrated under reduced pressure and purified by prep-HPLC to afford 2-((6-((5-chloro-2-((1R,5S,6s)-6-((4-(3-(2,6-dioxopiperidin-3-yl)-1-methyl-1H-indazol-7-yl)piperazin-1-yl)methyl)-3-azabicyclo[3.1.0]hexan-3-yl)pyrimidin-4-yl)amino)-1-methyl-2-oxo-1,2-dihydro-1,8-naphthyridin-3-yl)oxy)-N-methylacetamide (0.086 g) as an off white solid.

¹H NMR (400 MHz, DMSO-d6): δ 10.87 (s, 1H), 9.00 (s, 1H), 8.84 (d, J=2.40 Hz, 1H), 8.37 (d, J=2.40 Hz, 1H), 8.06 (s, 1H), 7.93 (d, J=4.40 Hz, 1H), 7.38 (dd, J=1.60, 7.00 Hz, 1H), 7.23 (s, 1H), 7.04-6.99 (m, 2H), 4.61 (s, 2H), 4.37-4.33 (m, 1H), 4.23 (s, 3H), 3.73 (s, 5H), 3.47-3.45 (m, 2H), 3.22-3.15 (m, 8H), 2.66-2.60 (m, 5H), 2.35-2.12 (m, 4H), 1.55 (s, 2H), 0.69 (br s, 1H).

LC-MS (ESI): m/z=795.58 [M+H]⁺.

### Example 31: 2-((6-((5-chloro-2-(4-((1-(3-(2,6-dioxopiperidin-3-yl)-1-methyl-1H-indazol-7-yl)piperidin-4-yl)methyl)piperidin-1-yl)pyrimidin-4-yl)amino)-1-methyl-2-oxo-1,2-dihydro-1,8-naphthyridin-3-yl)oxy)-N-methylacetamide (Compound 139)

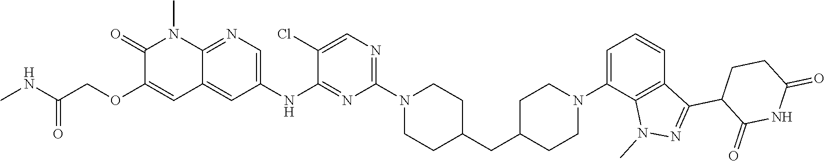 [SMILES: CNC(=O)COc1cc2cc(Nc3nc(N4CCC(CC5CCN(c6cccc7c(C8CCC(=O)NC8=O)nn(C)c67)CC5)CC4)ncc3Cl)cnc2n(C)c1=O]

### Preparation of tert-butyl 4-(pyridin-4-ylmethyl)piperidine-1-carboxylate

To a solution of tert-butyl 4-methylenepiperidine-1-carboxylate (2.0 g, 10.14 mmol) was added 9-BBN (10 mL) at 25° C. The reaction mixture was stirred at 80° C. for 1 h under N₂. After cooling, 4-bromopyridine (1.6 g, 10.14 mmol), Pd(dppf)Cl₂ (0.44 g, 0.61 mmol), K₂CO₃ (4.2 g, 30.42 mmol), DMF (16 mL), and H₂O (1.6 mL) were added. The resultant mixture was heated to 60° C. for 12 hours. After cooling, the reaction mixture was diluted with water (100 mL) and extracted with ethyl acetate (3×100 mL). The organic layer was washed with brine (2×100 mL), dried over sodium sulfate, and concentrated under reduced pressure to provide a crude product which was purified by silica gel column chromatography (0 to 40% ethyl acetate in petroleum ether) to afford tert-butyl 4-(pyridin-4-ylmethyl)piperidine-1-carboxylate (1.7 g) as a pale-yellow oil.

LC-MS (ESI): m/z=277.38 [M+H]⁺.

### Preparation of tert-butyl 4-(piperidin-4-ylmethyl)piperidine-1-carboxylate

To a solution of tert-butyl 4-(pyridin-4-ylmethyl)piperidine-1-carboxylate (1.7 g, 6.16 mmol) in THF (35 mL) and HOAc (0.2 mL) was added PtO₂ (850 mg, 50% w/w) at 25° C. Then the mixture was stirred at 60° C. for 24 h under H₂ (100 psi). After cooling, the reaction was filtered, and the filtrate was concentrated under reduced pressure to afford tert-butyl 4-(piperidin-4-ylmethyl)piperidine-1-carboxylate (2.80 g) as a brown oil which was used in the next step without further purification.

LC-MS (ESI): m/z=283.38 [M+H]⁺.

### Preparation of tert-butyl 4-((1-(3-(2,6-bis(benzyloxy)pyridin-3-yl)-1-methyl-1H-indazol-7-yl)piperidin-4-yl)methyl)piperidine-1-carboxylate

To a stirred solution of 3-(2,6-bis(benzyloxy)pyridin-3-yl)-7-bromo-1-methyl-1H-indazole (0.35 g, 0.7 mmol) and tert-butyl 4-(piperidin-3-yl)piperazine-1-carboxylate (0.6 g, 2.1 mmol) in 1,4-dioxane (7.0 mL) was added cesium carbonate (0.68 g, 2.1 mmol). The reaction was degassed with argon for 10 min followed by the addition of Pd-PEPPSI-IHeptCl (0.04 g, 0.04 mmol). The reaction mixture was stirred at 80° C. for 16 h. The reaction mixture was cooled to room temperature and filtered through a celite bed. The filtrate was concentrated to provide a crude product which was purified by flash column chromatography (SiO₂, 230-400 mesh, 40% ethyl acetate in petroleum ether) to afford tert-butyl 4-((1-(3-(2,6-bis(benzyloxy)pyridin-3-yl)-1-methyl-1H-indazol-7-yl)piperidin-4-yl)methyl)piperidine-1-carboxylate (0.28 g) as an off-white solid.

LC-MS (ESI): m/z=702.81 [M+H]⁺

### Preparation of tert-butyl 4-((1-(3-(2,6-dioxopiperidin-3-yl)-1-methyl-1H-indazol-7-yl)piperidin-4-yl)methyl)piperidine-1-carboxylate

To a stirred solution of tert-butyl 4-((1-(3-(2,6-bis(benzyloxy)pyridin-3-yl)-1-methyl-1H-indazol-7-yl)piperidin-4-yl)methyl)piperidine-1-carboxylate (0.28 g, 0.40 mmol) in THF (40 mL) was added 20% Pd(OH)₂ (0.28 g) at room temperature. The reaction was stirred at room temperature under H₂ atmosphere (60 psi) for 8 h. The reaction mixture was then filtered through a celite bed, washed with ethyl acetate, and concentrated under reduced pressure. The crude product was washed with n-pentane and dried to afford tert-butyl 4-((1-(3-(2,6-dioxopiperidin-3-yl)-1-methyl-1H-indazol-7-yl)piperidin-4-yl)methyl)piperidine-1-carboxylate (0.21 g) as a brown solid which was used in the next step without further purification.

LC-MS (ESI): m/z=524.70 [M+H]⁺.

### Preparation of 3-(1-methyl-7-(4-(piperidin-4-ylmethyl)piperidin-1-yl)-1H-indazol-3-yl)piperidine-2,6-dione

To a stirred solution of tert-butyl 4-((1-(3-(2,6-dioxopiperidin-3-yl)-1-methyl-1H-indazol-7-yl)piperidin-4-yl)methyl)piperidine-1-carboxylate (0.21 g, 0.4 mmol) in DCM (4.0 mL) was added TFA (2.0 mL) at 0° C. The reaction mixture was then stirred at room temperature for 3 h. The reaction mixture was concentrated under reduced pressure to provide a crude product which was triturated with diethyl ether (3 times) and dried to afford 3-(1-methyl-7-(4-(piperidin-4-ylmethyl)piperidin-1-yl)-1H-indazol-3-yl)piperidine-2,6-dione (0.016 g) as an off white solid.

LC-MS (ESI): m/z=424.52 [M+H]⁺

### Preparation of 2-((6-((5-chloro-2-(4-((1-(3-(2,6-dioxopiperidin-3-yl)-1-methyl-1H-indazol-7-yl)piperidin-4-yl)methyl)piperidin-1-yl)pyrimidin-4-yl)amino)-1-methyl-2-oxo-1,2-dihydro-1,8-naphthyridin-3-yl)oxy)-N-methylacetamide

To a stirred solution of 3-(1-methyl-7-(4-(piperidin-4-ylmethyl)piperidin-1-yl)-1H-indazol-3-yl)piperidine-2,6-dione (0.15 g, 0.35 mmol) and 2-((6-((2,5-dichloropyrimidin-4-yl)amino)-1-methyl-2-oxo-1,2-dihydro-1,8-naphthyridin-3-yl)oxy)-N-methylacetamide (0.06 g, 0.14 mmol) in DMSO (3 mL) was added N,N-diisopropylethylamine (0.5 mL, 2.8 mmol). The reaction mixture was stirred at 100° C. for 8 h. The reaction mixture was concentrated under reduced pressure and purified by prep-HPLC to afford 2-((6-((5-chloro-2-(4-((1-(3-(2,6-dioxopiperidin-3-yl)-1-methyl-1H-indazol-7-yl)piperidin-4-yl)methyl)piperidin-1-yl)pyrimidin-4-yl)amino)-1-methyl-2-oxo-1,2-dihydro-1,8-naphthyridin-3-yl)oxy)-N-methylacetamide (0.035 g) as an off white solid.

¹HNMR 400 MHz, DMSO-d6: δ 10.87 (s, 1H), 9.05 (s, 1H), 8.72 (s, 1H), 8.31 (s, 1H), 8.07 (s, 1H), 7.95 (s, 1H), 7.36 (s, 1H), 7.15 (s, 1H), 7.00 (s, 2H), 4.62 (s, 2H), 4.46 (d, J=9.20 Hz, 2H), 4.32 (s, 1H), 4.23 (s, 3H), 3.74 (s, 3H), 3.21-3.22 (m, 2H), 2.79-2.82 (m, 2H), 2.62-2.67 (m, 7H), 2.17-2.18 (m, 2H), 1.72-1.79 (m, 2H), 1.57-1.69 (m, 4H), 1.36-1.39 (m, 2H), 1.23 (br s, 2H), 1.03-1.05 (m, 2H).

LC-MS (ESI): m/z=794.47 [M+H]⁺

### Example 32: 2-((6-((5-chloro-2-(4-((1-(3-(2,6-dioxopiperidin-3-yl)-1-methyl-1H-indazol-6-yl)piperidin-4-yl)methyl)piperidin-1-yl)pyrimidin-4-yl)amino)-1-methyl-2-oxo-1,2-dihydro-1,8-naphthyridin-3-yl)oxy)-N-methylacetamide (Compound 140)

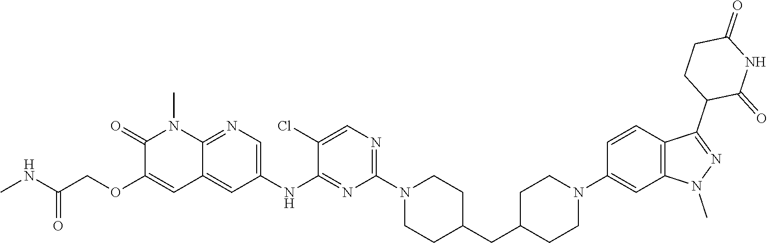 [SMILES: CNC(=O)COc1cc2cc(Nc3nc(N4CCC(CC5CCN(c6ccc7c(C8CCC(=O)NC8=O)nn(C)c7c6)CC5)CC4)ncc3Cl)cnc2n(C)c1=O]

### Preparation of tert-butyl 4-((1-(3-(2,6-bis(benzyloxy)pyridin-3-yl)-1-methyl-1H-indazol-6-yl)piperidin-4-yl)methyl)piperidine-1-carboxylate

To a stirred solution of 3-(2,6-bis(benzyloxy)pyridin-3-yl)-6-bromo-1-methyl-1H-indazole (0.50 g, 1.0 mmol) and tert-butyl 4-(piperidin-4-ylmethyl)piperidine-1-carboxylate (0.85 g, 3.0 mmol) in 1,4-dioxane (10 mL) was added cesium carbonate (0.98 g, 3.0 mmol). The reaction was degassed with argon for 10 minutes followed by the addition of RuPhos (0.05 g, 0.10 mmol) and RuPhos-PdG3 (0.04 g, 0.05 mmol). The reaction mixture was stirred at 80° C. for 16 h. The reaction mixture was cooled to room temperature and filtered through a celite bed. The filtrate was concentrated to provide a crude product which was purified by flash column chromatography (SiO₂, 230-400, 30% ethyl acetate in petroleum ether) to tert-butyl 4-((1-(3-(2,6-bis(benzyloxy)pyridin-3-yl)-1-methyl-1H-indazol-6-yl)piperidin-4-yl)methyl)piperidine-1-carboxylate (0.45 g) as an off white solid.

LC-MS (ESI): m/z=702.81 [M+H]⁺

### Preparation of tert-butyl 4-((1-(3-(2,6-dioxopiperidin-3-yl)-1-methyl-1H-indazol-6-yl)piperidin-4-yl)methyl)piperidine-1-carboxylate

To a stirred solution of tert-butyl 4-((1-(3-(2,6-bis(benzyloxy)pyridin-3-yl)-1-methyl-1H-indazol-6-yl)piperidin-4-yl)methyl)piperidine-1-carboxylate (0.45 g, 0.64 mmol) in THF (40 mL) was added 20% Pd(OH)₂ (0.45 g) at room temperature. The reaction was stirred at room temperature under H₂ (60 psi) atmosphere for 8 h. The reaction mixture was filtered through a celite bed, washed with ethyl acetate, and concentrated under reduced pressure. The crude product was washed with n-pentane and dried to afford tert-butyl 4-((1-(3-(2,6-dioxopiperidin-3-yl)-1-methyl-1H-indazol-6-yl)piperidin-4-yl)methyl)piperidine-1-carboxylate (0.30 g) as a brown solid.

LC-MS (ESI): m/z=524.65 [M+H]⁺.

### Preparation of 3-(1-methyl-6-(4-(piperidin-4-ylmethyl)piperidin-1-yl)-1H-indazol-3-yl)piperidine-2,6-dione

To a stirred solution of tert-butyl 4-((1-(3-(2,6-dioxopiperidin-3-yl)-1-methyl-1H-indazol-6-yl)piperidin-4-yl)methyl)piperidine-1-carboxylate (0.30 g, 0.57 mmol) in DCM (6.0 mL) cooled to 0° C. was added TFA (3.0 mL). The reaction mixture was then stirred at room temperature for 3 h. The reaction mixture was concentrated under reduced pressure to provide a crude product which was triturated with diethyl ether (3 times) and dried to afford 3-(1-methyl-6-(4-(piperidin-4-ylmethyl)piperidin-1-yl)-1H-indazol-3-yl)piperidine-2,6-dione (0.25 g) as an off white solid.

LC-MS (ESI): m/z=424.52 [M+H]⁺

### Preparation of 2-((6-((5-chloro-2-(4-((1-(3-(2,6-dioxopiperidin-3-yl)-1-methyl-1H-indazol-6-yl)piperidin-4-yl)methyl)piperidin-1-yl)pyrimidin-4-yl)amino)-1-methyl-2-oxo-1,2-dihydro-1,8-naphthyridin-3-yl)oxy)-N-methylacetamide

To a stirred solution of 3-(1-methyl-6-(4-(piperidin-4-ylmethyl)piperidin-1-yl)-1H-indazol-3-yl)piperidine-2,6-dione (0.15 g, 0.35 mmol) and 2-((6-((2,5-dichloropyrimidin-4-yl)amino)-1-methyl-2-oxo-1,2-dihydro-1,8-naphthyridin-3-yl)oxy)-N-methylacetamide (0.06 g, 0.14 mmol) in DMSO (3 mL) was added N,N-diisopropylethylamine (0.5 mL, 2.8 mmol). The reaction mixture was then stirred at 100° C. for 8 h. The reaction mixture was concentrated under reduced pressure and purified by prep-HPLC to afford 2-((6-((5-chloro-2-(4-((1-(3-(2,6-dioxopiperidin-3-yl)-1-methyl-1H-indazol-6-yl)piperidin-4-yl)methyl)piperidin-1-yl)pyrimidin-4-yl)amino)-1-methyl-2-oxo-1,2-dihydro-1,8-naphthyridin-3-yl)oxy)-N-methylacetamide (0.043 g) as an off white solid. ¹HNMR 400 MHz, DMSO-d6:10.84 (s, 1H), 9.04 (s, 1H), 8.72 (d, J=2.40 Hz, 1H), 8.30 (d, J=2.40 Hz, 1H), 8.07 (s, 1H), 7.94 (d, J=4.80 Hz, 1H), 7.47 (d, J=8.80 Hz, 1H), 7.15 (s, 1H), 6.89 (dd, J=1.60, 9.00 Hz, 1H), 6.81 (s, 1H), 4.61 (s, 2H), 4.45 (d, J=10.80 Hz, 2H), 4.23-4.24 (m, 1H), 3.88 (s, 3H), 3.73-3.75 (m, 5H), 2.80-2.83 (m, 2H), 2.64-2.65 (m, 6H), 2.59-2.61 (m, 1H), 2.31-2.20 (m, 1H), 2.14-2.16 (m, 1H), 1.68-1.71 (m, 6H), δ 1.17-1.19 (m, 4H), 1.02-1.04 (m, 2H).

LC-MS (ESI): m/z=794.47 [M+H]⁺

### Example 33: 2-((6-((5-chloro-2-(4-((1-(3-(2,4-dioxotetrahydropyrimidin-1 (2H)-yl)-1-methyl-1H-indazol-7-yl)piperidin-4-yl)methyl)piperazin-1-yl)pyrimidin-4-yl)amino)-1-isopropyl-2-oxo-1,2-dihydro-1,8-naphthyridin-3-yl)oxy)-N-methylacetamide (Compound 109)

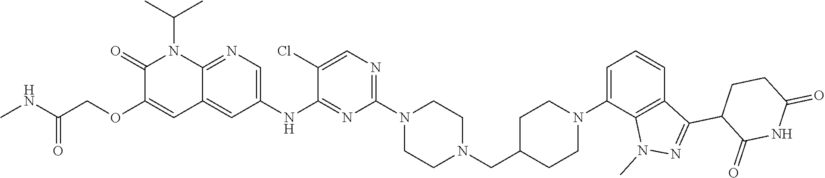 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)cnc2n(C(C)C)c1=O]

### Preparation of 2-((6-((5-chloro-2-(4-((1-(3-(2,4-dioxotetrahydropyrimidin-1 (2H)-yl)-1-methyl-1H-indazol-7-yl)piperidin-4-yl)methyl)piperazin-1-yl)pyrimidin-4-yl)amino)-1-isopropyl-2-oxo-1,2-dihydro-1,8-naphthyridin-3-yl)oxy)-N-methylacetamide

To a stirred solution of 1-(1-methyl-7-(4-(piperazin-1-ylmethyl)piperidin-1-yl)-1H-indazol-3-yl) dihydropyrimidine-2,4 (1H,3H)-dione (0.30 g, 0.70 mmol), 2-((6-((2,5-dichloropyrimidin-4-yl)amino)-1-isopropyl-2-oxo-1,2-dihydro-1,8-naphthyridin-3-yl)oxy)-N-methylacetamide (0.15 g, 0.35 mmol) in DMSO (1.80 mL), was added DIPEA (1.80 mL). The reaction mixture was then stirred and heated to 100° C. for 18 h. The reaction mixture was concentrated under reduced pressure and purified by prep-HPLC to afford 2-((6-((5-chloro-2-(4-((1-(3-(2,4-dioxotetrahydropyrimidin-1 (2H)-yl)-1-methyl-1H-indazol-7-yl)piperidin-4-yl)methyl) piperazin-1-yl)pyrimidin-4-yl)amino)-1-isopropyl-2-oxo-1,2-dihydro-1,8-naphthyridin-3-yl)oxy)-N-methylacetamide (0.057 g) as an off white solid.

¹H NMR (400 MHz, DMSO-d₆): δ 10.53 (s, 1H), 9.08 (s, 1H), 8.66 (d, J=2.40 Hz, 1H), 8.32 (d, J=2.40 Hz, 1H), 8.09 (s, 1H), 7.95 (d, J=4.80 Hz, 1H), 7.28-7.27 (m, 1H), 7.11 (s, 1H), 7.01 (d, J=4.80 Hz, 2H), 5.95 (br s, 1H), 4.59 (s, 2H), 4.23 (s, 3H), 3.8-3.86 (m, 2H), 3.63 (s, 4H), 3.27-3.24 (m, 2H), 2.75-2.74 (m, 2H), 2.68 (d, J=4.80 Hz, 5H), 2.41 (s, 4H), 2.25 (d, J=5.60 Hz, 2H), 1.89 (d, J=13.20 Hz, 2H), 1.57 (d, J=6.80 Hz, 6H), 1.40-1.37 (m, 3H).

LC-MS (ESI): m/z=826.52 [M+H]⁺.

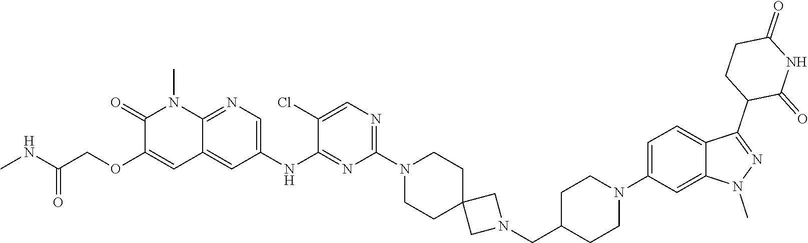 [SMILES: CNC(=O)COc1cc2cc(Nc3nc(N4CCC5(CC4)CN(CC4CCN(c6ccc7c(C8CCC(=O)NC8=O)nn(C)c7c6)CC4)C5)ncc3Cl)cnc2n(C)c1=O]

### Example 34: 2-((6-((5-chloro-2-(2-((1-(3-(2,6-dioxopiperidin-1 (2H)-yl)-1H-indazol-6-yl)piperidin-4-yl)methyl)-2,7-diazaspiro[3.5]nonan-7-yl)pyrimidin-4-yl)amino)-1-methyl-2-oxo-1,2-dihydro-1,8-naphthyridin-3-yl)oxy)-N-methylacetamide (Compound 109)

### Preparation of (1-(3-(2,6-bis(benzyloxy)pyridin-3-yl)-1-methyl-1H-indazol-6-yl)piperidin-4-yl)methanol

To a stirred solution of piperidin-4-ylmethanol (0.5 g, 4.34 mmol) and 3-(2,6-bis(benzyloxy)pyridin-3-yl)-6-bromo-1-methyl-1H-indazole (0.8 g, 1.73 mmol) in 1,4-dioxane (10 mL) was added cesium carbonate (4.2 g, 13.0 mmol). The reaction mixture was degassed with N₂ for 10 min. Pd-PEPPSI-iHept-Cl (0.20 g, 0.20 mmol) was then added, and the resultant reaction mixture was stirred at 100° C. for 3 h. After completion, the reaction mixture was filtered through a celite bed and washed with EtOAc (10 mL). The filtrate was concentrated to provide a crude product which was purified by flash column chromatography (60-120, 25% ethyl acetate in petroleum ether) to afford (1-(3-(2,6-bis(benzyloxy)pyridin-3-yl)-1-methyl-1H-indazol-6-yl)piperidin-4-yl)methanol (0.6 g) as a brown solid.

LC-MS (ESI): m/z=535.64 [M+H]⁺

### Preparation of (1-(3-(2,6-bis(benzyloxy)pyridin-3-yl)-1-methyl-1H-indazol-6-yl)piperidin-4-yl)methyl methane sulfonate

To a stirred solution of (1-(3-(2,6-bis(benzyloxy)pyridin-3-yl)-1-methyl-1H-indazol-6-yl)piperidin-4-yl)methanol (0.8 g, 1.49 mmol) and Et₃N (0.64 mL, 4.49 mmol) in DCM (16 mL) was added methanesulfonyl chloride (0.10 mL, 1.35 mmol) at 0° C. The reaction mixture was stirred at rt for 2 h. The reaction mixture was then filtered through a celite bed and concentrated under vacuum to provide a crude product which was washed with n-pentane (10 mL) and dried to afford (1-(3-(2,6-bis(benzyloxy)pyridin-3-yl)-1-methyl-1H-indazol-6-yl)piperidin-4-yl)methyl methane sulfonate (0.6 g)

LC-MS (ESI): 613.66 [M+H]⁺.

### Preparation of tert-butyl 2-((1-(3-(2,6-bis(benzyloxy)pyridin-3-yl)-1-methyl-1H-indazol-6-yl)piperidin-4-yl)methyl)-2,7-diazaspiro[3.5]nonane-7-carboxylate

To a stirred solution of (1-(3-(2,6-bis(benzyloxy)pyridin-3-yl)-1-methyl-1H-indazol-6-yl)piperidin-4-yl)methyl methane sulfonate (0.7 g, 1.14 mmol) and tert-butyl 2,7-diazaspiro[3.5]nonane-7-carboxylate (0.31 g, 1.37 mmol) in MeCN (14 mL) was added sodium carbonate (0.24 g, 2.28 mmol) and potassium iodide (0.03 g, 0.19 mmol). The resultant reaction mixture was stirred at 100° C. for 16 h. After completion of the reaction, the solvent was evaporated from the reaction mixture, and the resulting residue was diluted with water (10 mL) and extracted with EtOAc (3×20 mL). The extracts were combined, dried over anhydrous sodium sulfate, and concentrated under reduced pressure to provide a crude product which was purified by flash column chromatography (SiO₂, 60-120, 2% methanol in DCM) to afford tert-butyl 2-((1-(3-(2,6-bis(benzyloxy)pyridin-3-yl)-1-methyl-1H-indazol-6-yl)piperidin-4-yl)methyl)-2,7-diazaspiro[3.5]nonane-7-carboxylate (0.6 g) as a brown solid.

LC-MS (ESI): m/z=743.86 [M+H]⁺

### Preparation of tert-butyl 2-((1-(3-(2,6-dioxopiperidin-3-yl)-1-methyl-1H-indazol-6-yl) piperidin-4-yl)methyl)-2,7-diazaspiro[3.5]nonane-7-carboxylate

A stirred solution of tert-butyl 2-((1-(3-(2,6-bis(benzyloxy)pyridin-3-yl)-1-methyl-1H-indazol-6-yl)piperidin-4-yl)methyl)-2,7-diazaspiro[3.5]nonane-7-carboxylate (0.6 g, 0.80 mmol) in mixture of THF (18 mL), AcOH (0.06 mL) was degassed with nitrogen for 5 minutes. 20% Palladium hydroxide on carbon (0.71 g, 16.16 mmol) was then added. The reaction mixture was stirred under hydrogen atmosphere (15 Psi) at room temperature for 16 h. After completion, the reaction mixture was diluted with ethyl acetate (35 mL) and filtered through a pad of celite. The filtrate was collected and concentrated under reduced pressure to obtain a crude product which was purified by trituration with diethyl ether (12 mL) to afford tert-butyl 2-((1-(3-(2,6-dioxopiperidin-3-yl)-1-methyl-1H-indazol-6-yl)piperidin-4-yl)methyl)-2,7-diazaspiro[3.5]nonane-7-carboxylate (0.4 g).

LC-MS (ESI): m/z=565.70 [M+H]⁺

### Preparation of 3-(6-(4-((2,7-diazaspiro[3.5]nonan-2-yl)methyl)piperidin-1-yl)-1-methyl-1H-indazol-3-yl)piperidine-2,6-dione

To a stirred solution of tert-butyl 2-((1-(3-(2,6-dioxopiperidin-3-yl)-1-methyl-1H-indazol-6-yl)piperidin-4-yl)methyl)-2,7-diazaspiro[3.5]nonane-7-carboxylate (0.4 g, 0.7 mmol) in dichloromethane (4.0 mL) was added trifluoroacetic acid (2.0 mL) at 0° C. under N₂ atmosphere. The resultant reaction mixture was stirred at room temperature for 3 h. The reaction mixture was then concentrated under reduced pressure and co-distilled with DCM (10 ml) to provide a crude product which was purified by trituration with diethyl ether (10 ml) to afford 3-(6-(4-((2,7-diazaspiro[3.5]nonan-2-yl)methyl)piperidin-1-yl)-1-methyl-1H-indazol-3-yl)piperidine-2,6-dione (0.2 g) as an off white solid.

LC-MS (ESI): m/z=465.33 [M+H]⁺

### Preparation of 2-((6-((5-chloro-2-(2-((1-(3-(2,6-dioxopiperidin-3-yl)-1-methyl-1H-indazol-6-yl)piperidin-4-yl)methyl)-2,7-diazaspiro[3.5]nonan-7-yl)pyrimidin-4-yl)amino)-1-methyl-2-oxo-1,2-dihydro-1,8-naphthyridin-3-yl)oxy)-N-methylacetamide

To a stirred solution of 3-(6-(4-((2,7-diazaspiro[3.5]nonan-2-yl)methyl)piperidin-1-yl)-1-methyl-1H-indazol-3-yl)piperidine-2,6-dione (0.2 g, 0.43 mmol) and (2-((6-((2,5-dichloropyrimidin-4-yl)amino)-1-methyl-2-oxo-1,2-dihydro-1,8-naphthyridin-3-yl)oxy)-N-methylacetamide (0.07 g, 0.17 mmol) in DMSO (4.0 mL) was added DIPEA (0.38 mL, 2.19 mmol). The reaction mixture was then stirred at 100° C. for 3 h. Reaction mixture was cooled to room temperature and purified by prep-HPLC to afford 2-((6-((5-chloro-2-(2-((1-(3-(2,6-dioxopiperidin-3-yl)-1-methyl-1H-indazol-6-yl)piperidin-4-yl)methyl)-2,7-diazaspiro[3.5]nonan-7-yl)pyrimidin-4-yl)amino)-1-methyl-2-oxo-1,2-dihydro-1,8-naphthyridin-3-yl)oxy)-N-methylacetamide (0.045 g) as an off white solid

LC-MS (ESI): m/z=837.50 [M+H]⁺

¹H NMR (400 MHz, DMSO-d₆): δ 10.82 (s, 1H), 9.03 (d, J=27.60 Hz, 1H), 8.71 (d, J=2.40 Hz, 1H), 8.43 (d, J=8.80 Hz, 4H), 8.06 (s, 1H), 7.98 (d, J=4.00 Hz, 1H), 7.45 (d, J=8.80 Hz, 1H), 7.20-7.16 (m, 1H), 6.53 (d, J=7.60 Hz, 1H), 6.32 (s, 1H), 4.62-4.58 (m, 2H), 4.23-4.20 (m, 1H), 3.87 (s, 3H), 3.82 (s, 3H), 3.73 (s, 2H), 3.67 (s, 2H), 3.29-3.27 (m, 1H), 2.95-2.89 (m, 4H), 2.68-2.65 (m, 4H), 2.60-2.59 (m, 4H), 2.32-2.24 (m, 2H), 2.16-2.14 (m, 3H), 1.94-1.87 (m, 1H), 1.74-1.72 (m, 1H), 1.44-1.42 (m, 3H).

### Example 35: 2-((6-((5-chloro-2-(2-((1-(3-(2,6-dioxopiperidin-3-yl)-1-methyl-1H-indazol-7-yl)piperidin-4-yl)methyl)-2,7-diazaspiro[3.5]nonan-7-yl)pyrimidin-4-yl)amino)-1-methyl-2-oxo-1,2-dihydro-1,8-naphthyridin-3-yl)oxy)-N-methylacetamide (Compound 120)

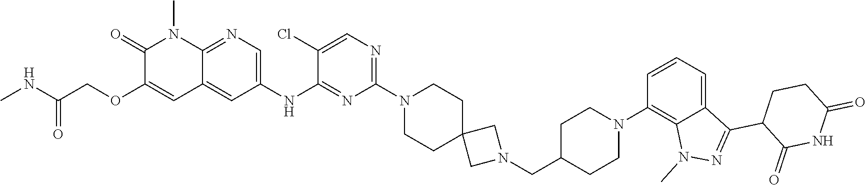 [SMILES: CNC(=O)COc1cc2cc(Nc3nc(N4CCC5(CC4)CN(CC4CCN(c6cccc7c(C8CCC(=O)NC8=O)nn(C)c67)CC4)C5)ncc3Cl)cnc2n(C)c1=O]

### Preparation of (1-(3-(2,6-bis(benzyloxy)pyridin-3-yl)-1-methyl-1H-indazol-7-yl) piperidin-4-yl)methanol

A solution of 3-(2,6-bis(benzyloxy)pyridin-3-yl)-7-bromo-1-methyl-1H-indazole (2.5 g, 4.99 mmol), piperidin-4-ylmethanol (1.72 g, 14.98 mmol), and cesium carbonate (5.69 g, 17.48 mmol) in 1,4-dioxane (50.0 mL) was degassed with argon for 10 minutes. Pd-PEPPSI-iHeptCl (0.24 g, 0.24 mmol) was then added to the reaction mixture. The resulting mixture was stirred and heated at 100° C. for 18 h. The reaction mixture was then diluted with DCM (300 mL), filtered through a celite bed, and the collected filtrate was concentrated under vacuum to obtain a residue which was purified by flash column chromatography (SiO₂, 230-400 mesh, 90% ethyl acetate in petroleum ether as eluent) to afford (1-(3-(2,6-bis(benzyloxy)pyridin-3-yl)-1-methyl-1H-indazol-7-yl)piperidin-4-yl)methanol (0.51 g) as a pale yellow solid.

LC-MS (ESI): m/z=535.60 [M+H]⁺.

### Preparation of (1-(3-(2,6-bis(benzyloxy)pyridin-3-yl)-1-methyl-1H-indazol-7-yl)piperidin-4-yl)methyl methanesulfonate

To a solution of (1-(3-(2,6-bis(benzyloxy)pyridin-3-yl)-1-methyl-1H-indazol-7-yl)piperidin-4-yl)methanol (0.20 g, 0.37 mmol) and triethylamine (0.09 g, 0.93 mmol) in DCM (2.0 mL) was added methanesulfonyl chloride (0.05 g, 0.44 mmol). The reaction mixture was stirred at room temperature for 2 h. The reaction mixture was poured into water (15 mL) and extracted with DCM (2×20 mL). The separated organic layers were combined, washed with brine (10 mL), dried over sodium sulfate, filtered, and concentrated under vacuum to obtain a residue which was triturated with n-pentane (5 mL) and dried under vacuum to afford (1-(3-(2,6-bis(benzyloxy)pyridin-3-yl)-1-methyl-1H-indazol-7-yl)piperidin-4-yl)methyl methanesulfonate (0.20 g) as a pale brown solid.

LC-MS (ESI): m/z=613.66 [M+H]⁺

### Preparation of tert-butyl 2-((1-(3-(2,6-bis(benzyloxy)pyridin-3-yl)-1-methyl-1H-indazol-7-yl)piperidin-4-yl)methyl)-2,7-diazaspiro[3.5]nonane-7-carboxylate

To a solution of (1-(3-(2,6-bis(benzyloxy)pyridin-3-yl)-1-methyl-1H-indazol-7-yl)piperidin-4-yl)methyl methanesulfonate (0.21 g, 0.34 mmol), tert-butyl 2,7-diazaspiro[3.5]nonane-7-carboxylate (0.11 g, 0.51 mmol), and sodium carbonate (0.14 g, 1.36 mmol) in MeCN (4.2 mL), was added potassium iodide (0.05 g, 0.03 mmol). The reaction mixture was stirred at 90° C. for 16 h. The reaction mixture was quenched with water (15 mL) and extracted with EtOAc (2×30 mL). The separated organic layers were washed with brine (10 mL), dried over anhydrous Na₂SO₄, filtered, concentrated under reduced pressure, and purified by flash column chromatography (SiO₂, 100-200 mesh; 5% methanol in dichloromethane as eluent) to afford tert-butyl 2-((1-(3-(2,6-bis(benzyloxy)pyridin-3-yl)-1-methyl-1H-indazol-7-yl)piperidin-4-yl)methyl)-2,7-diazaspiro[3.5]nonane-7-carboxylate (0.20 g) as a yellow semi-solid.

LC-MS (ESI): m/z=743.83 [M+H]⁺

### Preparation of tert-butyl 2-((1-(3-(2,6-dioxopiperidin-3-yl)-1-methyl-1H-indazol-7-yl) piperidin-4-yl)methyl)-2,7-diazaspiro[3.5]nonane-7-carboxylate

To a solution of tert-butyl 2-((1-(3-(2,6-bis(benzyloxy)pyridin-3-yl)-1-methyl-1H-indazol-7-yl)piperidin-4-yl)methyl)-2,7-diazaspiro[3.5]nonane-7-carboxylate (0.200 g, 0.27 mmol) and AcOH (0.2 mL) in THF (6.0 mL) was added 20% Pd(OH)₂/C (0.200 g, 100% w/w). The reaction mixture was stirred under hydrogen atmosphere (15 psi) at room temperature for 16 h. The reaction mixture was diluted with THF (30 mL), filtered through a celite bed, and washed with excess of THF:DCM (1:1, 150 mL). The collected filtrate was concentrated and dried under vacuum to afford tert-butyl 2-((1-(3-(2,6-dioxopiperidin-3-yl)-1-methyl-1H-indazol-7-yl)piperidin-4-yl)methyl)-2,7-diazaspiro[3.5]nonane-7-carboxylate (0.13 g) as a brown semi-solid which was used in the next step without further purification.

LC-MS (ESI): m/z=565.65 [M+H]⁺.

### Preparation of 3-(7-(4-((2,7-diazaspiro[3.5]nonan-2-yl)methyl)piperidin-1-yl)-1-methyl-1H-indazol-3-yl)piperidine-2,6-dione

To a solution of tert-butyl 2-((1-(3-(2,6-dioxopiperidin-3-yl)-1-methyl-1H-indazol-7-yl) piperidin-4-yl)methyl)-2,7-diazaspiro[3.5]nonane-7-carboxylate (0.13 g, 0.23 mmol) in DCM (2.6 mL) was added TFA (1.3 mL) at 0° C. The reaction mixture was stirred at room temperature for 3 h. The reaction mixture was concentrated under reduced pressure and co-distilled with DCM (2×10 mL) to afford 3-(7-(4-((2,7-diazaspiro[3.5]nonan-2-yl)methyl) piperidin-1-yl)-1-methyl-1H-indazol-3-yl)piperidine-2,6-dione (0.13 g) as a pale brown semi-solid.

LC-MS (ESI): m/z=465.55 [M+H]⁺.

### Preparation of 2-((6-((5-chloro-2-(2-((1-(3-(2,6-dioxopiperidin-3-yl)-1-methyl-1H-indazol-7-yl)piperidin-4-yl)methyl)-2,7-diazaspiro[3.5]nonan-7-yl)pyrimidin-4-yl)amino)-1-methyl-2-oxo-1,2-dihydro-1,8-naphthyridin-3-yl)oxy)-N-methylacetamide

To a stirred solution of 3-(7-(4-((2,7-diazaspiro[3.5]nonan-2-yl)methyl)piperidin-1-yl)-1-methyl-1H-indazol-3-yl)piperidine-2,6-dione (0.14 g, 0.30 mmol), 2-((6-((2,5-dichloropyrimidin-4-yl)amino)-1-methyl-2-oxo-1,2-dihydro-1,8-naphthyridin-3-yl)oxy)-N-methylacetamide (0.02 g, 0.06 mmol) in DMSO (2.8 mL) was added N, N-Diisopropylethylamine (1.4 mL). The reaction mixture was heated at 100° C. for 6 h. The reaction was concentrated under reduced pressure and purified by prep-HPLC to afford 2-((6-((5-chloro-2-(2-((1-(3-(2,6-dioxopiperidin-3-yl)-1-methyl-1H-indazol-7-yl)piperidin-4-yl)methyl)-2,7-diazaspiro[3.5]nonan-7-yl)pyrimidin-4-yl)amino)-1-methyl-2-oxo-1,2-dihydro-1,8-naphthyridin-3-yl)oxy)-N-methylacetamide (0.007 g) as an off white solid.

¹H NMR (400 MHz, DMSO-d₆): δ 10.88 (s, 1H), 9.06 (s, 1H), 8.71 (d, J=2.40 Hz, 1H), 8.28 (d, J=2.00 Hz, 1H), 8.06 (s, 1H), 7.98 (d, J=4.80 Hz, 1H), 7.35-7.35 (m, 1H), 7.17 (s, 1H), 7.00 (d, J=6.00 Hz, 1H), 6.94 (s, 1H), 4.62 (s, 2H), 4.32-4.31 (m, 1H), 4.22 (s, 3H), 3.74 (s, 3H), 3.59-3.57 (m, 4H), 3.22-3.21 (m, 2H), 2.97 (br s, 4H), 2.68 (d, J=4.80 Hz, 3H), 2.61-2.59 (m, 3H), 2.36-2.33 (m, 3H), 2.17-2.15 (m, 1H), 1.83-1.80 (m, 2H), 1.65 (br s, 4H), 1.39-1.34 (m, 4H).

LC-MS (ESI): m/z=837.50 [M+H]⁺.

### Example 36: 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-ethyl-2-oxo-1,2-dihydro-1,8-naphthyridin-3-yl)oxy)-N-methylacetamide (Compound 145)

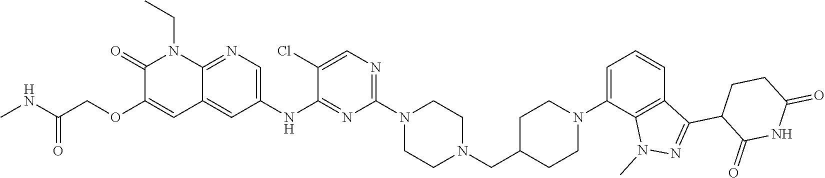 [SMILES: CCn1c(=O)c(OCC(=O)NC)cc2cc(Nc3nc(N4CCN(CC5CCN(c6cccc7c(C8CCC(=O)NC8=O)nn(C)c67)CC5)CC4)ncc3Cl)cnc21]

### Preparation of 1-ethyl-5-nitro-1H-pyrrolo[2,3-b]pyridine

To a stirred solution of 5-nitro-1H-pyrrolo[2,3-b]pyridine (1.0 g, 6.13 mmol) and K₂CO₃ (1.27 g, 9.19 mmol) in DMF (10 mL) was added ethyl iodide (1.43 g, 9.19 mmol). The reaction mixture was stirred at room temperature for 18 h. The reaction mixture was quenched with ice cold water (30 mL), and the precipitated solid was collected by filtration and dried under vacuum to obtain a crude product which was triturated with n-pentane (30 mL) to afford 1-ethyl-5-nitro-1H-pyrrolo[2,3-b]pyridine (0.90 g) as a pale brown semi-solid.

LC-MS (ESI): m/z=192.11 [M+H]⁺.

### Preparation of 1-ethyl-5-nitro-1H-pyrrolo[2,3-b]pyridine-2,3-dione

To a solution of 1-ethyl-5-nitro-1H-pyrrolo[2,3-b]pyridine (1.0 g, 5.23 mmol) in acetone (7 mL), was added CrO₃ (6.80 g, 68.03 mmol) in acetic acid (32 mL) and H₂O (10 mL). The reaction mixture was stirred at 0° C. for 3 h. The reaction mixture was poured into water (80 mL) and extracted with DCM (2×60 mL). The organic layer was separated, washed with brine (20 mL), dried over sodium sulfate, and concentrated to obtain a crude product which was triturated with n-pentane (30 mL) to afford 1-ethyl-5-nitro-1H-pyrrolo[2,3-b]pyridine-2,3-dione (3.50 g) as a pale brown semi-solid.

LC-MS (ESI): m/z=240.02 ([M+H]+18⁺)

### Preparation of 1-ethyl-3-methoxy-6-nitro-1,8-naphthyridin-2 (1H)-one

To a stirred solution of 1-ethyl-5-nitro-1H-pyrrolo[2,3-b]pyridine-2,3-dione (3.0 g, 13.57 mmol) and triethylamine (3.9 mL, 28.50 mmol) in ethanol (30 mL) was added TMSCH₂N₂ (2.0 M in DEE) (17 mL, 33.93 mmol) at 0° C. The reaction mixture was stirred at room temperature for 2 h. The reaction mixture was poured into water (1000 mL) and extracted with ethyl acetate (3×500 mL). The organic layer was separated, washed with brine (20 mL), dried over sodium sulfate, and concentrated to obtain a crude product which was triturated with diethyl ether (300 mL) and dried to afford 1-ethyl-3-methoxy-6-nitro-1,8-naphthyridin-2 (1H)-one (1.0 g) as a brown solid.

LC-MS (ESI): m/z=250.20 [M+H]⁺.

### Preparation of 1-ethyl-3-hydroxy-6-nitro-1,8-naphthyridin-2 (1H)-one

To a stirred solution of 1-ethyl-3-methoxy-6-nitro-1,8-naphthyridin-2 (1H)-one (0.6 g, 2.41 mmol) in DCM (12 mL) was added BBr₃ in DCM (1 M, 4.2 mL) dropwise at 0° C. The reaction mixture was stirred at room temperature for 2 h. The reaction mixture was quenched with methanol (50 mL) at 0° C., stirred for 30 minutes, and concentrated under reduced pressure to provide a crude product which was co-distilled with methanol (2×50 mL) and toluene (50 mL) to afford 1-ethyl-3-hydroxy-6-nitro-1,8-naphthyridin-2 (1H)-one (0.45 g) as a brown solid.

LC-MS (ESI): m/z=236.27 [M+H]⁺.

### Preparation of 2-((1-ethyl-6-nitro-2-oxo-1,2-dihydro-1,8-naphthyridin-3-yl)oxy)-N-methylacetamide

To a solution of 1-ethyl-3-hydroxy-6-nitro-1,8-naphthyridin-2 (1H)-one (0.25 g, 1.06 mmol) in DMF (2.5 mL) was added cesium carbonate (0.70 g, 2.13 mmol) and 2-bromo-N-methylacetamide (0.16 g, 1.06 mmol) at 0° C. The reaction mixture was stirred for 2 h. The reaction mixture was quenched with water (50 mL) at 0° C., and the precipitated solid was filtered under vacuum to provide a crude product which was triturated with petroleum ether (50 mL) and dried under vacuum to afford 2-((1-ethyl-6-nitro-2-oxo-1,2-dihydro-1,8-naphthyridin-3-yl)oxy)-N-methylacetamide (0.17 g) as a brown solid.

LC-MS (ESI): m/z=307.28 [M+H]⁺.

### Preparation of 2-((6-amino-1-ethyl-2-oxo-1,2-dihydro-1,8-naphthyridin-3-yl)oxy)-N-methylacetamide

To a stirred solution of 2-((1-ethyl-6-nitro-2-oxo-1,2-dihydro-1,8-naphthyridin-3-yl)oxy)-N-methylacetamide (0.15 g, 0.49 mmol) in methanol (1.5 mL) and water (1.5 mL), was added ammonium chloride (0.26 g, 4.90 mmol) and iron powder (0.26 g, 4.90 mmol). The reaction mixture was stirred at 65° C. for 3 h. The reaction mixture was diluted with DCM (30 mL), filtered through a pad of celite, and washed with an excess of MeOH in DCM (15%, 100 mL). The collected filtrate was concentrated under vacuum to obtain a residue which was diluted with MeOH in DCM (10%, 100 mL) and washed with water (3×30 mL). The separated organic layer was dried over anhydrous Na₂SO₄, filtered, and dried under vacuum to afford 2-((6-amino-1-ethyl-2-oxo-1,2-dihydro-1,8-naphthyridin-3-yl)oxy)-N-methylacetamide (0.12 g) as a pale green solid.

LC-MS (ESI): m/z=277.25 [M+H]⁺.

### Preparation of 2-((6-((2,5-dichloropyrimidin-4-yl)amino)-1-ethyl-2-oxo-1,2-dihydro-1,8-naphthyridin-3-yl)oxy)-N-methylacetamide

To a stirred solution of 2-((6-amino-1-ethyl-2-oxo-1,2-dihydro-1,8-naphthyridin-3-yl)oxy)-N-methylacetamide (0.14 g, 0.51 mmol) and DIPEA (0.70 mL, 4.06 mmol) in DMSO (2.8 mL) was added 2,4,5-trichloropyrimidine (0.1 g, 0.51 mmol). The reaction mixture was stirred at room temperature for 3 h. The reaction mixture was quenched with ice-cold water (30 mL), and the precipitated solid was filtered and dried under reduced pressure to provide a crude product which was triturated with DEE (50 mL), filtered, and dried under vacuum to afford 2-((6-((2,5-dichloropyrimidin-4-yl)amino)-1-ethyl-2-oxo-1,2-dihydro-1,8-naphthyridin-3-yl)oxy)-N-methylacetamide (0.14 g) as a brown solid.

LC-MS (ESI): m/z=423.30 [M+H]⁺.

### Preparation of 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-ethyl-2-oxo-1,2-dihydro-1,8-naphthyridin-3-yl)oxy)-N-methylacetamide

To a stirred solution of 2-((6-((2,5-dichloropyrimidin-4-yl)amino)-1-ethyl-2-oxo-1,2-dihydro-1,8-naphthyridin-3-yl)oxy)-N-methylacetamide (0.25 g, 0.59 mmol), 3-(1-methyl-7-(4-(piperazin-1-ylmethyl)piperidin-1-yl)-1H-indazol-3-yl)piperidine-2,6-dione (0.50 g, 1.18 mmol) in DMSO (2.5 mL) was added DIPEA (2.06 L, 11.81 mmol). The reaction mixture was stirred and heated at 120° C. for 20 h. The reaction mixture was concentrated under reduced pressure and purified by prep-HPLC to afford 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-ethyl-2-oxo-1,2-dihydro-1,8-naphthyridin-3-yl)oxy)-N-methylacetamide (0.07 g) as an off-white solid.

¹H NMR: (400 MHz, DMSO-d₆): δ 10.86 (s, 1H), 9.09 (s, 1H), 8.69 (d, J=2.80 Hz, 1H), 8.33 (d, J=2.40 Hz, 1H), 8.09 (s, 1H), 7.96 (d, J=4.40 Hz, 1H), 7.36 (dd, J=2.40, 6.40 Hz, 1H), 7.17 (s, 1H), 7.01-7.00 (m, 2H), 4.62 (s, 2H), 4.49-4.47 (m, 2H), 4.32-4.31 (m, 1H), 4.24 (s, 3H), 3.65 (br s, 4H), 3.25-3.23 (m, 2H), 2.67 (d, J=4.40 Hz, 3H), 2.62-2.61 (m, 4H), 2.40 (br s, 4H), 2.32-2.32 (m, 4H), 1.89-1.86 (m, 2H), 1.75-1.67 (m, 2H), 1.37-1.36 (m, 4H).

LC-MS (ESI): m/z=811.51 [M+H]⁺.

### Example 37: 2-((6-((5-chloro-2-((3S)-4-((1-(3-(2,6-dioxopiperidin-3-yl)-1-methyl-1H-indazol-7-yl)piperidin-4-yl)methyl)-3-(hydroxymethyl)piperazin-1-yl)pyrimidin-4-yl)amino)-1-isopropyl-2-oxo-1,2-dihydro-1,8-naphthyridin-3-yl)oxy)-N-methylacetamide (Compound 146a)

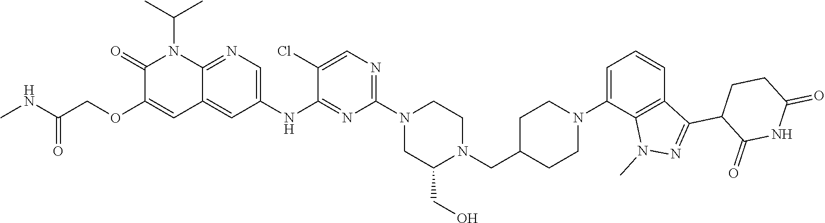 [SMILES: CNC(=O)COc1cc2cc(Nc3nc(N4CCN(CC5CCN(c6cccc7c(C8CCC(=O)NC8=O)nn(C)c67)CC5)[C@H](CO)C4)ncc3Cl)cnc2n(C(C)C)c1=O]

### Preparation of tert-butyl(S)-4-((1-((benzyloxy) carbonyl)piperidin-4-yl)methyl)-3-(hydroxymethyl)piperazine-1-carboxylate

To a solution of tert-butyl(S)-3-(hydroxymethyl)piperazine-1-carboxylate (2.0 g, 9.25 mmol) and benzyl 4-formylpiperidine-1-carboxylate (2.74 g, 11.10 mmol) in DCM (40 mL) was added acetic acid (0.55 g, 9.25 mmol) at 0° C. and stirred for 2 h. NaBH(OAc)₃ (3.92 g, 18.50) was then added at 0° C., and the reaction mixture was stirred at room temperature for an additional 16 h. The reaction mixture was quenched with ice-cold water (100 mL) and extracted with DCM (3×100 mL). The combined organic layers were washed with brine (100 mL), dried over anhydrous Na₂SO₄, filtered, dried under vacuum and purified by column chromatography (SiO₂, 230-400, 25% ethyl acetate in petroleum ether) to afford tert-butyl(S)-4-((1-((benzyloxy) carbonyl)piperidin-4-yl)methyl)-3-(hydroxymethyl)piperazine-1-carboxylate (4.0 g) as a colorless liquid.

LC-MS (ESI): m/z=448.54 [M+H]⁺

### Preparation of tert-butyl(S)-3-(hydroxymethyl)-4-(piperidin-4-ylmethyl)piperazine-1-carboxylate

To a solution of tert-butyl(S)-4-((1-((benzyloxy) carbonyl)piperidin-4-yl)methyl)-3-(hydroxymethyl)piperazine-1-carboxylate (4.0 g, 8.94 mmol) in THF (120 mL) was added 20% Pd(OH)₂ on carbon (4.0 g, 100% w/w). The reaction mixture was stirred under hydrogen atmosphere (70 psi) at room temperature for 16 h. The reaction mixture was filtered through a celite bed, concentrated under reduced pressure, washed with n-pentane and dried under vacuum to afford tert-butyl(S)-3-(hydroxymethyl)-4-(piperidin-4-ylmethyl)piperazine-1-carboxylate (2.0 g) as a brown solid which was used in the next step without purification.

LC-MS (ESI): m/z=314.51 [M+H]⁺

### Preparation of tert-butyl(S)-4-((1-(3-(2,6-bis(benzyloxy)pyridin-3-yl)-1-methyl-1H-indazol-7-yl)piperidin-4-yl)methyl)-3-(hydroxymethyl)piperazine-1-carboxylate

To a stirred solution of 3-(2,6-bis(benzyloxy)pyridin-3-yl)-7-bromo-1-methyl-1H-indazole (0.50 g, 1.00 mmol) and tert-butyl(S)-3-(hydroxymethyl)-4-(piperidin-4-ylmethyl)piperazine-1-carboxylate (0.62 g, 2.00 mmol) in 1,4-dioxane (10 mL) was added cesium carbonate (0.97 g, 3.00 mmol). The reaction mixture was degassed for 10 minutes. Afterwards, Pd PEPSI iHeptCl (0.04 g, 0.05 mmol) was added, and the resulting mixture was stirred at 100° C. for 12 h. The reaction mixture was then cooled to room temperature, filtered through a celite bed, concentrated under reduced pressure, and purified by flash column chromatography (SiO₂, 100-200, 25% ethyl acetate in petroleum ether) to afford tert-butyl (S)-4-((1-(3-(2,6-bis(benzyloxy)pyridin-3-yl)-1-methyl-1H-indazol-7-yl)piperidin-4-yl)methyl)-3-(hydroxymethyl)piperazine-1-carboxylate as a brown solid (0.4 g).

LC-MS (ESI): m/z=733.84 [M+H]⁺

### Preparation of tert-butyl (3S)-4-((1-(3-(2,6-dioxopiperidin-3-yl)-1-methyl-1H-indazol-7-yl)piperidin-4-yl)methyl)-3-(hydroxymethyl)piperazine-1-carboxylate

To a solution tert-butyl(S)-4-((1-(3-(2,6-bis(benzyloxy)pyridin-3-yl)-1-methyl-1H-indazol-7-yl)piperidin-4-yl)methyl)-3-(hydroxymethyl)piperazine-1-carboxylate (0.40 g, 0.54 mmol) in THF (16 mL) and AcOH (0.40 mL) was added 20% Pd(OH)₂ (0.40 g, 100% w/w). The reaction mixture was stirred under hydrogen atmosphere (70 psi) for 16 h at room temperature. The reaction mixture was diluted with THE, filtered through a pad of celite, and washed with excess of 20% THF in DCM. The filtrate was collected and concentrated to afford tert-butyl (3S)-4-((1-(3-(2,6-dioxopiperidin-3-yl)-1-methyl-1H-indazol-7-yl)piperidin-4-yl)methyl)-3-(hydroxymethyl)piperazine-1-carboxylate as a brown solid (0.30 g) which was used in the next step without purification.

LC-MS (ESI): m/z=555.71 [M+H]⁺.

### Preparation of 3-(7-(4-(((S)-2-(hydroxymethyl)-4-(2,2,2-trifluoroacetyl)piperazin-1-yl)methyl)piperidin-1-yl)-1-methyl-1H-indazol-3-yl)piperidine-2,6-dione

To a solution of tert-butyl (3S)-4-((1-(3-(2,6-dioxopiperidin-3-yl)-1-methyl-1H-indazol-7-yl)piperidin-4-yl)methyl)-3-(hydroxymethyl)piperazine-1-carboxylate (0.30 g, 0.54 mmol) in DCM (3.0 mL) was added TFA (1.5 mL, 5v). The reaction mixture was stirred for 2 h at room temperature. The reaction mixture was then concentrated and triturated with diethyl ether to afford 3-(7-(4-(((S)-2-(hydroxymethyl)-4-(2,2,2-trifluoroacetyl)piperazin-1-yl)methyl)piperidin-1-yl)-1-methyl-1H-indazol-3-yl)piperidine-2,6-dione (0.40 g) as TFA salt which was used in the next step without purification.

LC-MS (ESI): m/z=455.52 [M+H]⁺.

### Preparation of 2-((6-((5-chloro-2-((3S)-4-((1-(3-(2,6-dioxopiperidin-3-yl)-1-methyl-1H-indazol-7-yl)piperidin-4-yl)methyl)-3-(hydroxymethyl)piperazin-1-yl)pyrimidin-4-yl)amino)-1-isopropyl-2-oxo-1,2-dihydro-1,8-naphthyridin-3-yl)oxy)-N-methylacetamide

To a stirred solution of 3-(7-(4-(((S)-2-(hydroxymethyl)-4-(2,2,2-trifluoroacetyl)piperazin-1-yl)methyl)piperidin-1-yl)-1-methyl-1H-indazol-3-yl)piperidine-2,6-dione (0.20 g, 0.45 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.08 g, 0.18 mmol)) in DMSO (4 mL) was added DIPEA (0.63 mL, 3.64 mmol). The reaction mixture was heated at 100° C. for 12 h. The reaction mixture was quenched with ice-cold water. Then the solid thus formed was filtered and purified by prep-HPLC to afford 2-((6-((5-chloro-2-((3S)-4-((1-(3-(2,6-dioxopiperidin-3-yl)-1-methyl-1H-indazol-7-yl)piperidin-4-yl)methyl)-3-(hydroxymethyl)piperazin-1-yl)pyrimidin-4-yl)amino)-1-isopropyl-2-oxo-1,2-dihydro-1,8-naphthyridin-3-yl)oxy)-N-methylacetamide as an off-white solid (0.041 g).

¹H NMR 400 MHz, DMSO-d6: δ 10.90 (s, 1H), 9.05 (s, 1H), 8.65 (d, J=4.00 Hz, 1H), 8.46 (s, 1H), 8.10 (s, 1H), 7.93 (d, J=4.00 Hz, 1H), 7.36 (t, J=8.00 Hz, 1H), 7.15 (s, 1H), 7.03-6.99 (m, 2H), 5.95 (s, 1H), 4.56 (s, 3H), 4.33 (q, J=12.00 Hz, 1H), 4.24 (s, 3H), 3.86-3.85 (m, 1H), 3.67-3.70 (m, 1H), 3.32-3.20 (m, 4H), 2.87-2.84 (m, 1H), 2.68-2.60 (m, 8H), 2.38-2.37 (m, 1H), 2.32-2.15 (m, 4H), 1.94-1.92 (m, 1H), 1.83-1.80 (m, 1H), 1.56 (d, J=8.00 Hz, 6H), 1.39-1.35 (m, 2H). LC-MS (ESI): m/z=810.48 [M−H]⁻.

### Example 38: 2-((6-((5-chloro-2-(6-((1-(3-(2,6-dioxopiperidin-3-yl)-1-methyl-1H-indazol-7-yl)piperidin-4-yl)methyl)-2,6-diazaspiro[3.3]heptan-2-yl)pyrimidin-4-yl)amino)-1-methyl-2-oxo-1,2-dihydro-1,8-naphthyridin-3-yl)oxy)-N-methylacetamide (Compound

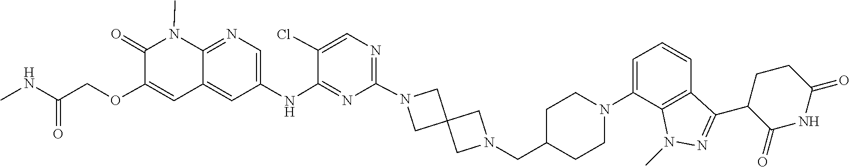 [SMILES: CNC(=O)COc1cc2cc(Nc3nc(N4CC5(CN(CC6CCN(c7cccc8c(C9CCC(=O)NC9=O)nn(C)c78)CC6)C5)C4)ncc3Cl)cnc2n(C)c1=O]

### Preparation of (1-(3-(2,6-bis(benzyloxy)pyridin-3-yl)-1-methyl-1H-indazol-7-yl)piperidin-4-yl)methanol

To a stirred solution of 3-(2,6-bis(benzyloxy)pyridin-3-yl)-7-bromo-1-methyl-1H-indazole (2.0 g, 3.99 mmol) in 1,4-dioxane (40 mL) was added 2-(piperidin-4-yl) ethan-1-ol (1.84 g, 15.9 mmol) and cesium carbonate (3.91 g, 12.0 mmol). The reaction was purged with nitrogen gas for 15 minutes, followed by the addition of Pd-Peppsi-iHeptCl (0.19 g, 0.20 mmol). The resulting mixture was stirred and heated at 100° C. for 16 h. The reaction mixture was then quenched with ice-cold water (30 mL) and extracted with ethyl acetate (3×150 mL). The combined organic layers were washed with brine (30 mL), dried over anhydrous Na₂SO₄, filtered, dried under vacuum, and purified by column chromatography (SiO₂, 100-200 mesh, 55% ethyl acetate in petroleum ether) to afford (1-(3-(2,6-bis(benzyloxy)pyridin-3-yl)-1-methyl-1H-indazol-7-yl)piperidin-4-yl)methanol (0.80 g) as a yellow solid.

LC-MS (ESI): m/z=535.57 [M+1]⁺

### Preparation of 3-(7-(4-(hydroxymethyl)piperidin-1-yl)-1-methyl-1H-indazol-3-yl)piperidine-2,6-dione

A solution of (1-(3-(2,6-bis(benzyloxy)pyridin-3-yl)-1-methyl-1H-indazol-7-yl)piperidin-4-yl)methanol (0.8 g, 1.49 mmol) in tetrahydrofuran (16 mL) was purged with N₂ for 5 min, followed by the addition of palladium on activated carbon (10%) (0.80 g, 100% w/w) at room temperature. The reaction mixture was then stirred at room temperature for 16 h under H₂ atmosphere (30 psi). The reaction mixture was then filtered through a celite bed, and the filtrate was concentrated under reduced pressure to afford 3-(7-(4-(hydroxymethyl)piperidin-1-yl)-1-methyl-1H-indazol-3-yl)piperidine-2,6-dione (0.50 g) which was used in the next step without further purification.

LC-MS (ESI): m/z=357.39 [M+H]⁺

### Preparation of 1-(3-(2,6-dioxopiperidin-3-yl)-1-methyl-1H-indazol-7-yl)piperidine-4-carbaldehyde

To a solution of 3-(7-(4-(hydroxymethyl)piperidin-1-yl)-1-methyl-1H-indazol-3-yl)piperidine-2,6-dione (0.50 g, 1.40 mmol) in DCM (10 mL) was added Dess-Martin periodinane (1.18 g, 2.80 mmol) at 0° C. under a N₂ atmosphere. The reaction mixture was stirred at room temperature for 4 h. The reaction mixture was quenched with saturated sodium bicarbonate (20 mL) and extracted with dichloromethane (2×100 mL). The separated organic layers were washed with brine (50 mL), dried over anhydrous sodium sulfate, filtered, and concentrated to afford 1-(3-(2,6-dioxopiperidin-3-yl)-1-methyl-1H-indazol-7-yl)piperidine-4-carbaldehyde (0.30 g) which was used in the next step without further purification.

LC-MS (ESI): m/z=355.38 [M+H]⁺

### Preparation of tert-butyl 6-((1-(3-(2,6-dioxopiperidin-3-yl)-1-methyl-1H-indazol-7-yl)piperidin-4-yl)methyl)-2,6-diazaspiro[3.3]heptane-2-carboxylate

A stirred solution of 1-(3-(2,6-dioxopiperidin-3-yl)-1-methyl-1H-indazol-7-yl)piperidine-4-carbaldehyde (0.30 g, 0.84 mmol) and tert-butyl 2,6-diazaspiro[3.3]heptane-2-carboxylate (0.33 g, 1.69 mmol) in DCM (10 mL) was purged with N₂ atmosphere for 1 h. After 1 h, Sodium cyanoborohydride (0.10 g, 1.69 mmol) was added at 0° C. The resulting mixture was stirred for 16 h at room temperature. The reaction mixture was then concentrated under reduced pressure, diluted with water (20 mL), and extracted with ethyl acetate (3×50 mL). The organic layers were concentrated under reduced pressure and purified by trituration with diethyl ether to afford tert-butyl 6-((1-(3-(2,6-dioxopiperidin-3-yl)-1-methyl-1H-indazol-7-yl)piperidin-4-yl)methyl)-2,6-diazaspiro[3.3]heptane-2-carboxylate (0.30 g).

LC-MS (ESI): m/z=537.37 [M+H]⁺.

### Preparation of 3-(7-(4-((2,6-diazaspiro[3.3]heptan-2-yl)methyl)piperidin-1-yl)-1-methyl-1H-indazol-3-yl)piperidine-2,6-dione

To a solution of tert-butyl 6-((1-(3-(2,6-dioxopiperidin-3-yl)-1-methyl-1H-indazol-7-yl)piperidin-4-yl)methyl)-2,6-diazaspiro[3.3]heptane-2-carboxylate (0.3 g, 0.55 mmol) in DCM (3 mL) was added TFA (1.5 mL, 5v) at 0° C. under N₂ atmosphere. The resultant reaction mixture was stirred at room temperature for 3 h. The reaction mixture was then concentrated under reduced pressure, co-distilled with DCM, and purified by trituration with diethyl ether (2×20 mL) to provide 3-(7-(4-((2,6-diazaspiro[3.3]heptan-2-yl)methyl)piperidin-1-yl)-1-methyl-1H-indazol-3-yl)piperidine-2,6-dione (0.30 g).

LC-MS (ESI): m/z=437.55 [M+H]⁺

### Preparation of 2-((6-((5-chloro-2-(6-((1-(3-(2,6-dioxopiperidin-3-yl)-1-methyl-1H-indazol-7-yl)piperidin-4-yl)methyl)-2,6-diazaspiro[3.3]heptan-2-yl)pyrimidin-4-yl)amino)-1-methyl-2-oxo-1,2-dihydro-1,8-naphthyridin-3-yl)oxy)-N-methylacetamide

To a stirred solution of 3-(7-(4-((2,6-diazaspiro[3.3]heptan-2-yl)methyl)piperidin-1-yl)-1-methyl-1H-indazol-3-yl)piperidine-2,6-dione (0.150 g, 0.34 mmol) and 2-((6-((2,5-dichloropyrimidin-4-yl)amino)-1-methyl-2-oxo-1,2-dihydro-1,8-naphthyridin-3-yl)oxy)-N-methylacetamide (0.07 g, 0.17 mmol) in DMSO (3 mL) was added N,N-diisopropylethylamine (0.17 mL, 1.02 mmol) at room temperature and heated at 100° C. for 3 h. The reaction mixture was concentrated under reduced pressure and purified by prep-HPLC to afford 2-((6-((5-chloro-2-(6-((1-(3-(2,6-dioxopiperidin-3-yl)-1-methyl-1H-indazol-7-yl)piperidin-4-yl)methyl)-2,6-diazaspiro[3.3]heptan-2-yl)pyrimidin-4-yl)amino)-1-methyl-2-oxo-1,2-dihydro-1,8-naphthyridin-3-yl)oxy)-N-methylacetamide (0.0109 g) as an off white solid.

¹H NMR (400 MHz, DMSO-d₆): δ 10.90 (s, 1H), 9.57 (br s, 1H), 9.12 (s, 1H), 8.91 (br s, 1H), 8.25 (br s, 1H), 8.10 (s, 1H), 7.38 (t, J=4.4 Hz, 1H), 7.26 (s, 1H), 7.02 (d, J=4.4 Hz, 2H), 4.6 (s, 2H) 4.4-4.2 (m, 8H), 4.10 (s, 2H), 3.56 (s, 3H), 3.32-3.24 (m, 7H), 2.99 (s, 2H), 2.67-2.63 (m, 2H), 2.60-2.50 (m, 2H), 1.81-1.78 (m, 2H), 1.35-1.27 (m, 3H), 1.25 (s, 2H).

LC-MS (ESI): m/z=807.51 [M−H]⁻.

### Example 39: 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-cyclopropyl-2-oxo-1,2-dihydro-1,8-naphthyridin-3-yl)oxy)-N-methylacetamide (Compound 150)

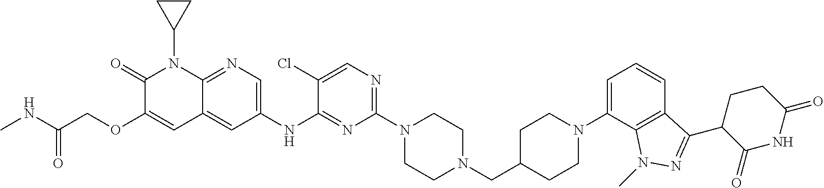 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)cnc2n(C2CC2)c1=O]

### Preparation of 1-cyclopropyl-5-nitro-1H-pyrrolo[2,3-b]pyridine

To a solution of 5-nitro-1H-pyrrolo[2,3-b]pyridine (2.0 g, 12.26 mmol), cyclopropyl boronic acid (2.10 g, 24.53 mmol), sodium carbonate (2.60 g, 24.53 mmol), and 2,2′-bi pyridine (1.91 g, 12.26 mmol) in DCM (60.0 mL) was added copper (II) acetate (2.22 g, 12.26 mmol). The reaction mixture was stirred and heated to 40° C. for 18 h. The reaction mixture was diluted with DCM (300 mL) and filtered through a celite bed. The collected filtrate was concentrated under vacuum to provide a crude product which was purified by flash column chromatography (SiO₂, 230-400, 40% ethyl acetate in petroleum ether as eluent) to afford 1-cyclopropyl-5-nitro-1H-pyrrolo[2,3-b]pyridine (1.51 g) as a pale yellow solid.

LC-MS (ESI): m/z=204.12 [M+H]⁺.

### Preparation of 1-cyclopropyl-5-nitro-1H-pyrrolo[2,3-b]pyridine-2,3-dione

To a solution of 1-cyclopropyl-5-nitro-1H-pyrrolo[2,3-b]pyridine (1.5 g, 7.38 mmol) in acetone (45 mL) was added a solution of CrO₃ (9.60 g, 96.02 mmol) in acetic acid (48 mL) and H₂O (15 mL). The reaction mixture was stirred for 3 h at 0° C. The reaction mixture was poured into water (100 mL) and extracted with DCM (2×20 mL). The separated organic layer was washed with brine (10 mL), dried over sodium sulfate, filtered, and concentrated under vacuum to afford 1-cyclopropyl-5-nitro-1H-pyrrolo[2,3-b]pyridine-2,3-dione (0.90 g) as a pale-yellow solid which was used in the next step without purification.

LC-MS (ESI): m/z=252.18 [M+H]+18⁺.

### Preparation of 1-cyclopropyl-3-methoxy-6-nitro-1,8-naphthyridin-2 (1H)-one

To a stirred solution of 1-cyclopropyl-5-nitro-1H-pyrrolo[2,3-b]pyridine-2,3-dione (0.90 g, 3.86 mmol) in Ethanol (27 mL) was added triethylamine (1.12 mL, 8.1 mmol) and TMSCH₂N₂ (2.0 M in DEE) (9.0 mL, 0.70 mmol). The reaction mixture was stirred at room temperature for 3 h. The reaction mixture was filtered, washed with petroleum ether (50 mL), and dried under vacuum to provide a crude product which was triturated with diethyl ether (100 mL) and dried to afford 1-cyclopropyl-3-methoxy-6-nitro-1,8-naphthyridin-2 (1H)-one (0.27 g) as a pale brown solid.

LC-MS (ESI): m/z=262.38 [M+H]⁺.

### Preparation of 1-cyclopropyl-3-hydroxy-6-nitro-1,8-naphthyridin-2 (1H)-one

To a cooled solution of 1-cyclopropyl-3-methoxy-6-nitro-1,8-naphthyridin-2 (1H)-one (0.1 g, 0.38 mmol) in DCM (2.0 mL) was added BBr₃ in DCM (1.0 M) solution (1.0 mL, 3.82 mmol) dropwise at 0° C. The reaction mixture was then stirred at room temperature for 2 h. The reaction mixture was quenched with methanol (100 mL) at 0° C. and concentrated under vacuum to afford 1-cyclopropyl-3-hydroxy-6-nitro-1,8-naphthyridin-2 (1H)-one (0.10 g) as a pale brown solid which was used in the next step without purification.

LC-MS (ESI): m/z=248.36 [M+H]⁺.

### Preparation of 2-((1-cyclopropyl-6-nitro-2-oxo-1,2-dihydro-1,8-naphthyridin-3-yl)oxy)-N-methylacetamide

To a solution of 1-cyclopropyl-3-hydroxy-6-nitro-1,8-naphthyridin-2 (1H)-one (0.10 g, 0.405 mmol) and cesium carbonate (0.39 g, 1.21 mmol) in DMF (2.0 mL) was added 2-bromo-N-methylacetamide (0.15 g, 1.01 mmol). The reaction mixture was stirred at room temperature for 1 h. The reaction mixture was quenched with ice cold water (20 mL) and the precipitated solid was filtered, washed with petroleum ether (100 mL), and dried under vacuum to afford 2-((1-cyclopropyl-6-nitro-2-oxo-1,2-dihydro-1,8-naphthyridin-3-yl)oxy)-N-methylacetamide (0.09 g) as a pale-brown solid which was used in the next step without purification.

LC-MS (ESI): m/z=319.43 [M+H]⁺.

### Preparation of 2-((6-amino-1-cyclopropyl-2-oxo-1,2-dihydro-1,8-naphthyridin-3-yl)oxy)-N-methylacetamide

To a stirred solution of 2-((1-cyclopropyl-6-nitro-2-oxo-1,2-dihydro-1,8-naphthyridin-3-yl)oxy)-N-methylacetamide (0.09 g, 0.28 mmol) in methanol (3.0 mL) and H₂O (2.0 mL) was added NH₄Cl (0.15 g, 2.82 mmol) and iron (0.15 g, 2.81 mmol). The reaction mixture was stirred and heated at 70° C. for 4 h. The reaction mixture was diluted with methanol (200 mL) and filtered through a pad of celite. The collected filtrate was dried under vacuum to afford 2-((6-amino-1-cyclopropyl-2-oxo-1,2-dihydro-1,8-naphthyridin-3-yl)oxy)-N-methylacetamide (0.08 g) as a pale-brown solid which was used in the next step without purification.

LC-MS (ESI): m/z=289.31 [M+H]⁺.

### Preparation of 2-((1-cyclopropyl-6-((2,5-dichloropyrimidin-4-yl)amino)-2-oxo-1,2-dihydro-1,8-naphthyridin-3-yl)oxy)-N-methylacetamide

To a stirred solution of a 2-((6-amino-1-cyclopropyl-2-oxo-1,2-dihydro-1,8-naphthyridin-3-yl)oxy)-N-methylacetamide (0.08 g, 0.27 mmol) and DIPEA (0.6 mL) in DMSO (1.20 mL) was added 2,4,5-trichloropyrimidine (0.06 g, 0.32 mmol). The reaction mixture was stirred and heated at 100° C. for 8 h. The reaction mixture was quenched with ice cold water (20 mL), and the precipitated solid was filtered and dried under vacuum to afford 2-((1-cyclopropyl-6-((2,5-dichloropyrimidin-4-yl)amino)-2-oxo-1,2-dihydro-1,8-naphthyridin-3-yl)oxy)-N-methylacetamide (0.07 g) as a pale brown semi-solid which was used in the next step without purification.

LC-MS (ESI): m/z=435.39 [M+H]⁺.

### Preparation of 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-cyclopropyl-2-oxo-1,2-dihydro-1,8-naphthyridin-3-yl)oxy)-N-methylacetamide

To stirred solution of a 2-((1-cyclopropyl-6-((2,5-dichloropyrimidin-4-yl)amino)-2-oxo-1,2-dihydro-1,8-naphthyridin-3-yl)oxy)-N-methylacetamide (0.07 g, 0.16 mmol) and 3-(1-methyl-7-(4-(piperazin-1-ylmethyl)piperidin-1-yl)-1H-indazol-3-yl)piperidine-2,6-dione (0.075 g, 0.17 mmol) in DMSO (0.7 mL) was added DIPEA (0.5 mL). The reaction mixture was stirred at 100° C. for 8 h. The reaction mixture was concentrated under reduced pressure and purified by prep-HPLC to afford 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-cyclopropyl-2-oxo-1,2-dihydro-1,8-naphthyridin-3-yl)oxy)-N-methylacetamide (0.052 g) as an off-white solid.

¹H NMR: (400 MHz, DMSO-d₆): δ 10.88 (s, 1H), 9.07 (s, 1H), 8.64 (d, J=2.40 Hz, 1H), 8.27 (d, J=2.40 Hz, 1H), 8.09 (s, 1H), 7.94 (d, J=4.80 Hz, 1H), 7.36 (dd, J=2.40, 6.60 Hz, 1H), 7.11 (s, 1H), 7.02 (d, J=7.60 Hz, 1H), 7.00 (s, 1H), 4.58 (s, 2H), 4.32-4.31 (m, 1H), 4.24 (s, 3H), 3.62 (s, 4H), 3.24-3.22 (m, 2H), 3.02-3.01 (m, 1H), 2.67 (d, J=4.40 Hz, 3H), 2.62-2.61 (m, 4H), 2.50 (s, 4H), 2.25-2.24 (m, 3H), 2.17-2.15 (m, 2H), 1.88 (d, J=10.80 Hz, 2H), 1.39-1.36 (m, 2H), 1.22-1.21 (m, 2H), 0.83-0.81 (m, 2H).

LC-MS (ESI): m/z=823.51 [M+H]⁺.

### Example 40: 2-((6-((5-chloro-2-((2R)-4-((1-(3-(2,6-dioxopiperidin-3-yl)-1-methyl-1H-indazol-6-yl)piperidin-4-yl)methyl)-2-(hydroxymethyl)piperazin-1-yl)pyrimidin-4-yl)amino)-1-isopropyl-2-oxo-1,2-dihydro-1,8-naphthyridin-3-yl)oxy)-N-methylacetamide (Compound 151a)

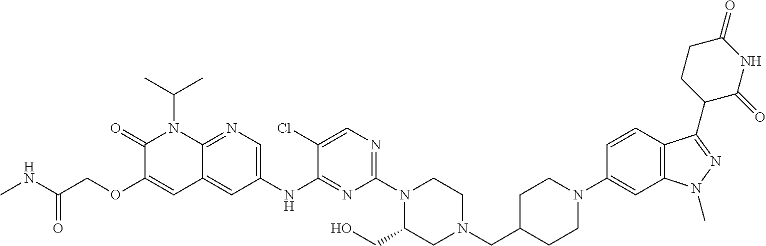 [SMILES: CNC(=O)COc1cc2cc(Nc3nc(N4CCN(CC5CCN(c6ccc7c(C8CCC(=O)NC8=O)nn(C)c7c6)CC5)C[C@@H]4CO)ncc3Cl)cnc2n(C(C)C)c1=O]

### Preparation of tert-butyl (R)-4-((1-((benzyloxy) carbonyl)piperidin-4-yl)methyl)-2-(hydroxymethyl)piperazine-1-carboxylate

To a stirred solution of tert-butyl (R)-2-(hydroxymethyl)piperazine-1-carboxylate (2 g, 9.24 mmol) and benzyl 4-formylpiperidine-1-carboxylate (2.2 g, 9.24 mmol) in DCM (40 mL) was added acetic acid (0.52 mL, 9.24 mmol), followed by the addition of sodium triacetoxyborohydride (2.35 g, 11.09 mmol) at 0° C. under a nitrogen atmosphere. The reaction mixture was stirred at room temperature for 16 h. The reaction mixture was quenched with water (200 mL) and extracted with ethyl acetate (2×100 mL). The combined organic layers were washed with brine, dried over anhydrous Na₂SO₄, filtered, and concentrated under reduced pressure to provide a crude product which was purified by flash column chromatography (SiO₂, 60-120 mesh, 25% ethyl acetate in petroleum ether) to afford tert-butyl (R)-4-((1-((benzyloxy) carbonyl)piperidin-4-yl)methyl)-2-(hydroxymethyl)piperazine-1-carboxylate (2.8 g) as a yellow semi-solid.

LC-MS (ESI): m/z=448.35 [M+H]⁺.

### Preparation of tert-butyl-(R)-2-(hydroxymethyl)-4-(piperidin-4-ylmethyl)piperazine-1-carboxylate

To a stirred solution of tert-butyl (R)-4-((1-((benzyloxy) carbonyl)piperidin-4-yl)methyl)-2-(hydroxymethyl)piperazine-1-carboxylate (2.8 g, 6.26 mmol) in THF (84 mL) was added 20% Pd(OH)₂ on carbon, moisture 50% wet (2.8 g, 100% w/w). The reaction was stirred under hydrogen atmosphere (80 psi) at room temperature for 16 h. The reaction mixture was diluted with DCM (50 mL), filtered through a celite bed, and washed with 30% THF in DCM (500 mL). The collected filtrate was concentrated under reduced pressure and azeotroped with toluene to afford tert-butyl-(R)-2-(hydroxymethyl)-4-(piperidin-4-ylmethyl)piperazine-1-carboxylate (1.5 g) as an off white solid.

¹H NMR, 400 MHz, DMSO-d6: δ 4.64 (br s, 1H), 3.86 (s, 1H), 3.62-3.60 (m, 2H), 3.40-3.38 (m, 1H), 3.08 (d, J=12.00 Hz, 2H), 1.86-1.85 (m, 2H), 2.88-2.79 (m, 2H), 2.07-2.06 (m, 3H), 1.72-1.68 (m, 6H), 1.35 (s, 9H), 1.23-1.22 (m, 2H).

### Preparation of tert-butyl (R)-4-((1-(3-(2,6-bis(benzyloxy)pyridin-3-yl)-1-methyl-1H-indazol-6-yl)piperidin-4-yl)methyl)-2-(hydroxymethyl)piperazine-1-carboxylate

To a stirred solution of 3-(2,6-bis(benzyloxy)pyridin-3-yl)-6-bromo-1-methyl-1H-indazole (0.6 g, 1.19 mmol) and tert-butyl-(R)-2-(hydroxymethyl)-4-(piperidin-4-ylmethyl)piperazine-1-carboxylate (1.12 g, 3.59 mmol) in a mixture of 1,4-dioxane (12 mL) and DMF (3.0 mL) was added cesium carbonate (1.55 g, 4.76 mmol). The solution was degassed with argon for 15 minutes followed by the addition of Ruphos (0.11 g, 0.23 mmol) and Ruphos Pd G3 (0.09 g, 0.11 mmol). The mixture was purged again for 2 minutes. The resulting reaction mixture was heated to 100° C. for 4 h. The reaction mixture was then diluted with EtOAc (20 mL), filtered through a celite bed, and washed with ethyl acetate (100 mL). The collected filtrate was concentrated under reduced pressure to provide a crude product which was purified by flash column chromatography (SiO₂, 100-200 mesh, 30% ethyl acetate in petroleum ether) to afford tert-butyl (R)-4-((1-(3-(2,6-bis(benzyloxy)pyridin-3-yl)-1-methyl-1H-indazol-6-yl)piperidin-4-yl)methyl)-2-(hydroxymethyl)piperazine-1-carboxylate (0.62 g) as a brown semi-solid.

LC-MS (ESI): m/z=733.71 [M+H]⁺.

### Preparation of tert-butyl(2R)-4-((1-(3-(2,6-dioxopiperidin-3-yl)-1-methyl-1H-indazol-6-yl)piperidin-4-yl)methyl)-2-(hydroxymethyl)piperazine-1-carboxylate

To a stirred solution of tert-butyl (R)-4-((1-(3-(2,6-bis(benzyloxy)pyridin-3-yl)-1-methyl-1H-indazol-6-yl)piperidin-4-yl)methyl)-2-(hydroxymethyl)piperazine-1-carboxylate (0.62 g, 0.84 mmol) in THF (18.6 mL) was added 20% Pd(OH)₂ on carbon, moisture 50% wet (1.24 g). The reaction was stirred under hydrogen atmosphere (80 Psi) at room temperature for 16 h. The reaction mixture was diluted with DCM (50 mL), filtered through a celite bed, and washed with 30% THF in DCM (100 mL). The collected filtrate was concentrated under reduced pressure to provide a crude product which was triturated by using diethyl ether to afford tert-butyl(2R)-4-((1-(3-(2,6-dioxopiperidin-3-yl)-1-methyl-1H-indazol-6-yl)piperidin-4-yl)methyl)-2-(hydroxymethyl)piperazine-1-carboxylate (0.52 g) as an off white solid.

LC-MS (ESI): m/z=555.59 [M+H]⁺.

### Preparation of 3-(6-(4-(((R)-3-(hydroxymethyl)piperazin-1-yl)methyl)piperidin-1-yl)-1-methyl-1H-indazol-3-yl)piperidine-2,6-dione

To a stirred solution of tert-butyl(2R)-4-((1-(3-(2,6-dioxopiperidin-3-yl)-1-methyl-1H-indazol-6-yl)piperidin-4-yl)methyl)-2-(hydroxymethyl)piperazine-1-carboxylate (0.52 g, 0.93 mmol) in DCM (5.2 mL) was added TFA (2.6 mL). The reaction was stirred at room temperature for 1 h. The reaction mixture was concentrated under reduced pressure and co-distilled with petroleum ether under reduced pressure to provide a crude product which was triturated by using diethyl ether to afford 3-(6-(4-(((R)-3-(hydroxymethyl)piperazin-1-yl)methyl)piperidin-1-yl)-1-methyl-1H-indazol-3-yl)piperidine-2,6-dione (0.36 g).

LC-MS (ESI): m/z=455.57 [M+H]⁺.

### Preparation of 2-((6-((5-chloro-2-((2R)-4-((1-(3-(2,6-dioxopiperidin-3-yl)-1-methyl-1H-indazol-6-yl)piperidin-4-yl)methyl)-2-(hydroxymethyl)piperazin-1-yl)pyrimidin-4-yl)amino)-1-isopropyl-2-oxo-1,2-dihydro-1,8-naphthyridin-3-yl)oxy)-N-methylacetamide

To a stirred solution of 3-(6-(4-(((R)-3-(hydroxymethyl)piperazin-1-yl)methyl)piperidin-1-yl)-1-methyl-1H-indazol-3-yl)piperidine-2,6-dione (0.18 g, 0.39 mmol) in DMSO (3.6 mL) was added 2-((6-((2,5-dichloropyrimidin-4-yl)amino)-1-isopropyl-2-oxo-1,2-dihydro-1,8-naphthyridin-3-yl)oxy)-N-methylacetamide (0.06 g, 0.15 mmol) and DIPEA (0.34 mL, 1.95 mmol). The reaction mixture was stirred and heated to 100° C. for 16 h. The reaction mixture was concentrated under reduced pressure and purified by prep-HPLC to afford 2-((6-((5-chloro-2-((2R)-4-((1-(3-(2,6-dioxopiperidin-3-yl)-1-methyl-1H-indazol-6-yl)piperidin-4-yl)methyl)-2-(hydroxymethyl)piperazin-1-yl)pyrimidin-4-yl)amino)-1-isopropyl-2-oxo-1,2-dihydro-1,8-naphthyridin-3-yl)oxy)-N-methylacetamide (0.009 g) as an off-white solid.

LC-MS (ESI): m/z=853.51 [M−H]⁻.

¹H-NMR (400 MHz, DMSO-d₆): δ 10.85 (s, 1H), 9.07 (s, 1H), 8.63-8.55 (m, 2H), 7.99 (s, 1H), 8.08 (s, 1H), 7.48 (d, J=8.80 Hz, 1H), 7.21 (s, 1H), 6.83-6.87 (m, 2H), 5.93 (s, 1H), 4.23-4.24 (m, 5H), 3.79-3.85 (m, 7H), 3.32 (br, 1H), 3.02-3.09 (m, 2H), 2.62-2.64 (m, 7H), 2.51-2.59 (m, 2H), 2.31-2.32 (m, 1H), 2.13-2.17 (m, 3H), 1.81-1.85 (m, 7H), 1.60-1.55 (m, 3H), 1.35-1.28 (m, 2H).

### Example 41: 2-((6-((5-chloro-2-((2S)-4-((1-(3-(2,6-dioxopiperidin-3-yl)-1-methyl-1H-indazol-6-yl)piperidin-4-yl)methyl)-2-(hydroxymethyl)piperazin-1-yl)pyrimidin-4-yl)amino)-1-isopropyl-2-oxo-1,2-dihydro-1,8-naphthyridin-3-yl)oxy)-N-methylacetamide (Compound 151b)

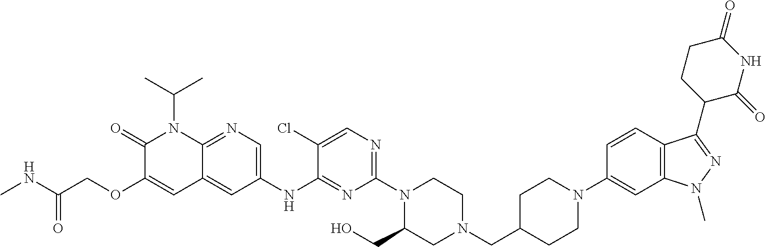 [SMILES: CNC(=O)COc1cc2cc(Nc3nc(N4CCN(CC5CCN(c6ccc7c(C8CCC(=O)NC8=O)nn(C)c7c6)CC5)C[C@H]4CO)ncc3Cl)cnc2n(C(C)C)c1=O]

### Preparation of tert-butyl(S)-4-((1-((benzyloxy) carbonyl)piperidin-4-yl)methyl)-2-(hydroxymethyl)piperazine-1-carboxylate

To a solution of tert-butyl(S)-2-(hydroxymethyl)piperazine-1-carboxylate (2.0 g, 9.24 mmol), benzyl 4-formylpiperidine-1-carboxylate (2.28 g, 9.24 mmol) in DCM (40 mL) was added acetic acid (0.5 mL, 9.24 mmol). The reaction mixture was stirred at room temperature for 6 h, and then NaBH(OAc)₃ (2.35 g, 11.09 mmol) was added. The resulting mixture was stirred at room temperature for 16 h. The reaction mixture was quenched with ice-cold water (100 mL) and extracted into DCM (3×100 mL). The combined organic layers were washed with brine (100 mL), dried over anhydrous Na₂SO₄, filtered, and dried under vacuum to get a residue which was purified by column chromatography (SiO₂ 100-200 mesh; 40% ethyl acetate in petroleum ether) to afford tert-butyl(S)-4-((1-((benzyloxy) carbonyl)piperidin-4-yl)methyl)-2-(hydroxymethyl)piperazine-1-carboxylate (4.0 g) as a white solid.

LC-MS (ESI): m/z=448.32 [M+H]⁺.

### Preparation of tert-butyl-(S)-2-(hydroxymethyl)-4-(piperidin-4-ylmethyl)piperazine-1-carboxylate

To a solution of tert-butyl(S)-4-((1-((benzyloxy) carbonyl)piperidin-4-yl)methyl)-2-(hydroxymethyl)piperazine-1-carboxylate (4.0 g, 8.93 mmol) in THF (240 mL) was added 10% palladium on carbon (4.2 g, 40.21 mmol). The reaction mixture was stirred under hydrogen atmosphere (60 psi) at room temperature for 16 h. The reaction mixture was filtered through a celite bed, washed with ethyl acetate (2×100 mL), concentrated under reduced pressure, washed with n-pentane, and dried to afford tert-butyl-(S)-2-(hydroxymethyl)-4-(piperidin-4-ylmethyl)piperazine-1-carboxylate (2.5 g,) as a white solid.

LC-MS (ESI): m/z=314.51 [M+H]⁺.

### Preparation of tert-butyl-(S)-4-((1-(3-(2,6-bis(benzyloxy)pyridin-3-yl)-1-methyl-1H-indazol-6-yl)piperidin-4-yl)methyl)-2-(hydroxymethyl)piperazine-1-carboxylate

A solution of 3-(2,6-bis(benzyloxy)pyridin-3-yl)-6-bromo-1-methyl-1H-indazole (1.0 g, 1.99 mmol), tert-butyl-(S)-2-(hydroxymethyl)-4-(piperidin-4-ylmethyl)piperazine-1-carboxylate (1.5 g, 4.99 mmol) and cesium carbonate (1.9 g, 5.99 mmol) in 1,4-dioxane (20 mL) was degassed with argon for 15 minutes. Afterwards, Ruphos (0.09 g, 0.20 mmol) and RuPhos Pd G3 (0.08 g, 0.10 mmol) were added and the reaction mixture was heated at 100° C. for 16 h. The reaction mixture was diluted with ice-cold water (100 mL) and extracted into ethyl acetate (3×100 mL). The combined organic layers were washed with brine solution (100 mL), dried over anhydrous Na₂SO₄, filtered, and dried under vacuum to get a residue which was purified by column chromatography (SiO₂ 100-200 mesh; 40% ethyl acetate in petroleum ether) to afford tert-butyl-(S)-4-((1-(3-(2,6-bis(benzyloxy)pyridin-3-yl)-1-methyl-1H-indazol-6-yl)piperidin-4-yl)methyl)-2-(hydroxymethyl)piperazine-1-carboxylate (0.50 g) as a pale brown solid.

LC-MS (ESI): m/z=[M+H]⁺

### Preparation of tert-butyl-(2S)-4-((1-(3-(2,6-dioxopiperidin-3-yl)-1-methyl-1H-indazol-6-yl)piperidin-4-yl)methyl)-2-(hydroxymethyl)piperazine-1-carboxylate

To a solution of tert-butyl-(S)-4-((1-(3-(2,6-bis(benzyloxy)pyridin-3-yl)-1-methyl-1H-indazol-6-yl)piperidin-4-yl)methyl)-2-(hydroxymethyl)piperazine-1-carboxylate (0.48 g, 0.65 mmol) in THF (36.0 mL) was added 20% Pd(OH)₂ on carbon (0.48 g, 100% w/w). The reaction mixture was stirred under hydrogen atmosphere (60 psi) at room temperature for 6 h. The reaction mixture was filtered through a celite bed, washed with ethyl acetate (100 mL), concentrated under reduced pressure, washed with n-pentane, and dried to afford tert-butyl-(2S)-4-((1-(3-(2,6-dioxopiperidin-3-yl)-1-methyl-1H-indazol-6-yl)piperidin-4-yl)methyl)-2-(hydroxymethyl)piperazine-1-carboxylate (0.34 g,) as a green solid.

LC-MS (ESI): m/z=555.67 [M+1]⁺.

### Preparation of 3-(6-(4-(((S)-3-(hydroxymethyl)piperazin-1-yl)methyl)piperidin-1-yl)-1-methyl-1H-indazol-3-yl)piperidine-2,6-dione

To a solution of tert-butyl-(2S)-4-((1-(3-(2,6-dioxopiperidin-3-yl)-1-methyl-1H-indazol-6-yl)piperidin-4-yl)methyl)-2-(hydroxymethyl)piperazine-1-carboxylate (0.38 g, 0.68 mmol) in DCM (4.0 mL), was added TFA (2.0 mL) at 0° C. and stirred at room temperature for 3 h. The reaction mixture was concentrated under reduced pressure and washed with n-pentane to afford 3-(6-(4-(((S)-3-(hydroxymethyl)piperazin-1-yl)methyl)piperidin-1-yl)-1-methyl-1H-indazol-3-yl)piperidine-2,6-dione (0.25 g) as a white solid.

LC-MS (ESI): m/z=455.61 [M+H]⁺

### Preparation of 2-((6-((5-chloro-2-((2S)-4-((1-(3-(2,6-dioxopiperidin-3-yl)-1-methyl-1H-indazol-6-yl)piperidin-4-yl)methyl)-2-(hydroxymethyl)piperazin-1-yl)pyrimidin-4-yl)amino)-1-isopropyl-2-oxo-1,2-dihydro-1,8-naphthyridin-3-yl)oxy)-N-methylacetamide

To a solution 3-(6-(4-(((S)-3-(hydroxymethyl)piperazin-1-yl)methyl)piperidin-1-yl)-1-methyl-1H-indazol-3-yl)piperidine-2,6-dione (0.11 g, 0.24 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.05 g, 0.12 mmol) in DMSO was added N,N-diisopropylethylamine (0.2 mL, 1.21 mmol). The reaction mixture was stirred at 100° C. for 16 h. The reaction mixture was poured into ice cold water (15 mL) and stirred for 15 minutes. The resulting precipitate was filtered, dried, and purified by prep-HPLC to afford 2-((6-((5-chloro-2-((2S)-4-((1-(3-(2,6-dioxopiperidin-3-yl)-1-methyl-1H-indazol-6-yl)piperidin-4-yl)methyl)-2-(hydroxymethyl)piperazin-1-yl)pyrimidin-4-yl)amino)-1-isopropyl-2-oxo-1,2-dihydro-1,8-naphthyridin-3-yl)oxy)-N-methylacetamide (5 mg) as a white solid.

LC-MS (ESI): m/z=853.47 [M+1]⁺.

¹HNMR, 400 MHz, DMSO: δ 10.80 (s, 1H), 9.06 (s, 1H), 8.20-8.80 (m, 2H), 8.08 (s, 1H), 7.91 (d, J=4.80 Hz, 1H), 7.48 (d, J=8.80 Hz, 1H), 7.20 (s, 1H), 6.90-6.83 (m, 2H), 5.94 (br s, 1H), 4.95-4.65 (m, 1H), 4.64-4.22 (m, 5H), 3.93-3.80 (m, 6H), 3.60-3.50 (m, 1H), 3.08-3.03 (m, 2H), 2.86-2.84 (m, 1H), 2.73-2.71 (m, 1H), 2.62-2.61 (m, 5H), 2.17-2.13 (m, 3H), 1.81-1.71 (m, 5H), 1.57-1.55 (m, 5H), 1.35-1.25 (m, 5H).

### Example 42: 2-((6-((5-chloro-2-(4-((4-(3-(2,6-dioxopiperidin-3-yl)-1-methyl-1H-indazol-7-yl)piperazin-1-yl)methyl)piperidin-1-yl)pyridin-4-yl)amino)-1-isopropyl-2-oxo-1,2-dihydro-1,8-naphthyridin-3-yl)oxy)-N-methylacetamide (Compound 152)

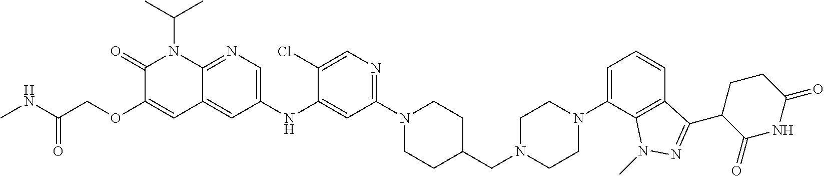 [SMILES: CNC(=O)COc1cc2cc(Nc3cc(N4CCC(CN5CCN(c6cccc7c(C8CCC(=O)NC8=O)nn(C)c67)CC5)CC4)ncc3Cl)cnc2n(C(C)C)c1=O]

### Preparation of (1-(5-chloro-4-iodopyridin-2-yl)piperidin-4-yl)methanol

To a stirred solution of 5-chloro-2-fluoro-4-iodopyridine (0.60 g, 2.33 mmol) and piperidin-4-ylmethanol (0.27 g, 2.33 mmol) in DMSO (6.0 mL) was added DIPEA (3.2 mL, 18.64 mmol). The reaction was then stirred at 100° C. for 4 h. The reaction mixture was cooled to room temperature, quenched with cold water (10 mL), and extracted with ethyl acetate (2×15 mL). The combined organic layer was dried over Na₂SO₄, filtered, and dried to obtain a crude product which was purified by flash chromatography (230-400 silica gel; 20% ethyl acetate in petroleum ether) to afford (1-(5-chloro-4-iodopyridin-2-yl)piperidin-4-yl)methanol (0.70 g) as a pale yellow solid.

LC-MS (ESI): m/z=353.19 [M+H]⁺

### Preparation of 2-((6-((5-chloro-2-(4-(hydroxymethyl)piperidin-1-yl)pyridin-4-yl)amino)-1-isopropyl-2-oxo-1,2-dihydro-1,8-naphthyridin-3-yl)oxy)-N-methylacetamide

To a stirred solution of (1-(5-chloro-4-iodopyridin-2-yl)piperidin-4-yl)methanol (0.60 g, 1.70 mmol) and 2-((6-amino-1-isopropyl-2-oxo-1,2-dihydro-1,8-naphthyridin-3-yl)oxy)-N-methylacetamide (0.49 g, 1.70 mmol) in 1,4-dioxane (18 mL) was added cesium carbonate (1.66 g, 5.11 mmol). The reaction mixture was purged with argon for 10 minutes. Afterwards, Ruphos (0.08 g, 0.17 mmol) was added followed by Ruphos Pd G3 (0.07 g, 0.09 mmol). The reaction mixture was again purged with argon for 2 minutes. The reaction mixture was stirred at 90° C. for 16 h. The reaction mixture was diluted with ethyl acetate (50 mL) and washed with 10% MeOH:DCM (150 mL). The filtrate was concentrated and dried to obtain a crude product which was purified by flash chromatography (230-400 silica gel; 0-10% MeOH in DCM) followed by reverse-phase HPLC to afford 2-((6-((5-chloro-2-(4-(hydroxymethyl)piperidin-1-yl)pyridin-4-yl)amino)-1-isopropyl-2-oxo-1,2-dihydro-1,8-naphthyridin-3-yl)oxy)-N-methylacetamide as an off-white solid (0.21 g).

LC-MS (ESI): m/z=515.54 [M+H]⁺.

### Preparation of 2-((6-((5-chloro-2-(4-formylpiperidin-1-yl)pyridin-4-yl)amino)-1-isopropyl-2-oxo-1,2-dihydro-1,8-naphthyridin-3-yl)oxy)-N-methylacetamide

To a stirred solution of 2-((6-((5-chloro-2-(4-(hydroxymethyl)piperidin-1-yl)pyridin-4-yl)amino)-1-isopropyl-2-oxo-1,2-dihydro-1,8-naphthyridin-3-yl)oxy)-N-methylacetamide (0.20 g, 0.38 mmol) in DCM (20.0 mL) was added Dess-Martin periodinane (0.65 g, 1.55 mmol) at 0° C. The resulting reaction mixture was stirred at room temperature for 16 h. The reaction mixture was quenched with NaHCO₃ solution (20 mL) and extracted with DCM (2×20 mL). The combined organic layer was dried over anhydrous Na₂SO₄, filtered, and concentrated to afford crude product 2-((6-((5-chloro-2-(4-formylpiperidin-1-yl)pyridin-4-yl)amino)-1-isopropyl-2-oxo-1,2-dihydro-1,8-naphthyridin-3-yl)oxy)-N-methylacetamide as a brown solid (0.21 g) which was used in the next step without further purification.

LC-MS (ESI): m/z=513.48 [M+H]⁺

### Preparation of tert-butyl 4-(3-(2,6-bis(benzyloxy)pyridin-3-yl)-1-methyl-1H-indazol-7-yl)piperazine-1-carboxylate

To a stirred solution of 3-(2,6-bis(benzyloxy)pyridin-3-yl)-7-bromo-1-methyl-1H-indazole (0.60 g, 1.20 mmol) and tert-butyl piperazine-1-carboxylate (0.55 g, 3.00 mmol) in 1,4-dioxane (18.0 mL) was added cesium carbonate (1.17 g, 3.60 mmol). The reaction mixture was degassed with argon for 15 min. Afterwards, Pd-PPEPSI-ihept Cl (0.06 g, 0.06 mmol) was added, and the reaction mixture was stirred at 100° C. for 16 h. The reaction mixture was cooled to room temperature, diluted with ethyl acetate (50 mL), filtered through a celite bed, and washed with 10% MeOH:DCM (200 mL). The filtrate was concentrated under reduced pressure to obtain a crude product which was purified by flash chromatography (230-400 silica gel; 10-30% ethyl acetate in petroleum ether) to afford tert-butyl 4-(3-(2,6-bis(benzyloxy)pyridin-3-yl)-1-methyl-1H-indazol-7-yl)piperazine-1-carboxylate as a yellow solid (0.38 g).

LC-MS (ESI): m/z=606.65 [M+H]⁺

### Preparation of tert-butyl 4-(3-(2,6-dioxopiperidin-3-yl)-1-methyl-1H-indazol-7-yl)piperazine-1-carboxylate

To a solution of tert-butyl 4-(3-(2,6-bis(benzyloxy)pyridin-3-yl)-1-methyl-1H-indazol-7-yl)piperazine-1-carboxylate (0.38 g, 0.62 mmol) in THF (23.0 mL) (in a Parr shaker), was added 20% Pd(OH)₂/C (0.38 g, 100% w/w). The reaction mixture was stirred under hydrogen atmosphere (85 psi) at room temperature for 16 h. The reaction mixture was diluted with THE (30 mL), filtered through a celite bed, and washed with THF:DCM (1:1, 200 mL). The filtrate was concentrated and dried to provide tert-butyl 4-(3-(2,6-dioxopiperidin-3-yl)-1-methyl-1H-indazol-7-yl)piperazine-1-carboxylate (0.31 g) as a black solid which was used in the next step without further purification.

LC-MS (ESI): m/z=428.51 [M+H]⁺

### Preparation of 3-(1-methyl-7-(piperazin-1-yl)-1H-indazol-3-yl)piperidine-2,6-dione

To a stirred solution of tert-butyl 4-(3-(2,6-dioxopiperidin-3-yl)-1-methyl-1H-indazol-7-yl)piperazine-1-carboxylate (0.31 g, 0.72 mmol) in DCM (6.2 mL) was added TFA (3.1 mL) at 0° C. The reaction mixture was stirred at room temperature for 3 h. The reaction mixture was concentrated under reduced pressure and co-distilled with DCM (2×10 mL) to provide a crude product which was triturated with diethyl ether (2×10 mL) to afford 3-(1-methyl-7-(piperazin-1-yl)-1H-indazol-3-yl)piperidine-2,6-dione as a brown semi solid (0.38 g).

LC-MS (ESI): m/z=328.48 [M+H]⁺

### Preparation of 2-((6-((5-chloro-2-(4-((4-(3-(2,6-dioxopiperidin-3-yl)-1-methyl-1H-indazol-7-yl)piperazin-1-yl)methyl)piperidin-1-yl)pyridin-4-yl)amino)-1-isopropyl-2-oxo-1,2-dihydro-1,8-naphthyridin-3-yl)oxy)-N-methylacetamide

To a stirred solution of 2-((6-((5-chloro-2-(4-formylpiperidin-1-yl)pyridin-4-yl)amino)-1-isopropyl-2-oxo-1,2-dihydro-1,8-naphthyridin-3-yl)oxy)-N-methylacetamide (0.10 g, 0.20 mmol) and 3-(1-methyl-7-(piperazin-1-yl)-1H-indazol-3-yl)piperidine-2,6-dione (0.08 g, 0.23 mmol) in DCM (6.0 mL) was added triethylamine (0.50 mL). The reaction mixture was stirred at 40° C. for 1 h followed by the addition of sodium triacetoxyborohydride (0.12 g, 0.60 mmol) at 0° C. The reaction mixture was stirred at room temperature for 4 h. The reaction mixture was concentrated under reduced pressure to obtain a crude product which was purified by prep-HPLC to afford 2-((6-((5-chloro-2-(4-((4-(3-(2,6-dioxopiperidin-3-yl)-1-methyl-1H-indazol-7-yl)piperazin-1-yl)methyl)piperidin-1-yl)pyridin-4-yl)amino)-1-isopropyl-2-oxo-1,2-dihydro-1,8-naphthyridin-3-yl)oxy)-N-methylacetamide (0.05 g) as an off white solid.

¹H NMR (400 MHz, DMSO-d₆): δ 10.85 (s, 1H), 8.46-8.42 (m, 2H), 7.97-7.95 (m, 3H), 7.39-7.37 (m, 1H), 7.23 (s, 1H), 7.02-6.99 (m, 2H), 6.21 (s, 1H), 5.97 (s, 1H), 4.55 (s, 2H), 4.37-4.33 (m, 1H), 4.23 (s, 3H), 4.04 (d, J=12.00 Hz, 2H), 3.17-2.87 (m, 6H), 2.72-2.62 (m, 7H), 2.37-2.14 (m, 6H), 1.73 (d, J=9.60 Hz, 3H), 1.57 (d, J=6.80 Hz, 6H), 1.13-1.03 (m, 2H).

LC-MS (ESI): m/z=822.49 [M−H]⁻

### Example 43: 2-((6-((5-chloro-2-((3R)-4-((1-(3-(2,6-dioxopiperidin-3-yl)-1-methyl-1H-indazol-7-yl)piperidin-4-yl)methyl)-3-(hydroxymethyl)piperazin-1-yl)pyrimidin-4-yl)amino)-1-isopropyl-2-oxo-1,2-dihydro-1,8-naphthyridin-3-yl)oxy)-N-methylacetamide (Compound 146b)

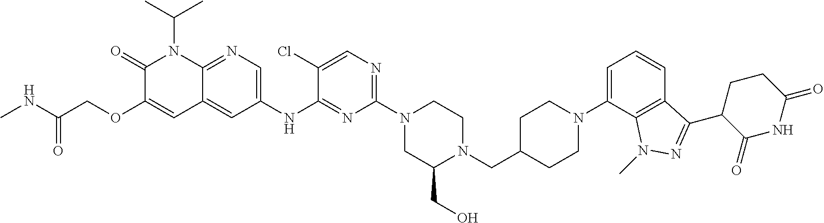 [SMILES: CNC(=O)COc1cc2cc(Nc3nc(N4CCN(CC5CCN(c6cccc7c(C8CCC(=O)NC8=O)nn(C)c67)CC5)[C@@H](CO)C4)ncc3Cl)cnc2n(C(C)C)c1=O]

### Preparation of tert-butyl-(R)-4-((1-((benzyloxy) carbonyl)piperidin-4-yl)methyl)-3-(hydroxymethyl)piperazine-1-carboxylate

To a solution of tert-butyl-(R)-3-(hydroxymethyl)piperazine-1-carboxylate (4.00 g, 18.49 mmol), benzyl4-formylpiperidine-1-carboxylate (4.57 g, 18.49 mmol), and acetic acid (1.06 mL, 18.49 mmol) in DCM (160 mL) was added sodium triacetoxyborohydride (5.88 g, 27.741 mmol) portion wise. The reaction mixture was stirred at room temperature for 18 h. The reaction mixture was quenched with ice-cold water (200 mL) and extracted with DCM (3×200 mL). The combined organic layers were washed with brine (200 mL), dried over anhydrous Na₂SO₄, filtered, dried under vacuum, and purified by column chromatography (SiO₂; 40% ethyl acetate in petroleum ether as eluent) to afford tert-butyl-(R)-4-((1-((benzyloxy) carbonyl)piperidin-4-yl)methyl)-3-(hydroxymethyl)piperazine-1-carboxylate (7.90 g) as a colorless semi-solid.

LC-MS (ESI): m/z=448.54 [M+H]⁺

### Preparation of tert-butyl-(R)-3-(hydroxymethyl)-4-(piperidin-4-ylmethyl)piperazine-1-carboxylate

To a solution of tert-butyl-(R)-4-((1-((benzyloxy) carbonyl)piperidin-4-yl)methyl)-3-(hydroxymethyl)piperazine-1-carboxylate (0.78 g, 1.06 mmol) and acetic acid (0.78 mL) in THF (40 mL) was added 20% Pd(OH)₂ on carbon (1.05 g, 7.45 mmol). The reaction mixture was stirred under hydrogen atmosphere (80 psi) at room temperature for 18 h. The reaction mixture was filtered through a celite bed, concentrated under vacuum, washed with n-pentane, dried to afford tert-butyl-(R)-3-(hydroxymethyl)-4-(piperidin-4-ylmethyl)piperazine-1-carboxylate (0.70 g) as an off-white solid.

### Preparation of tert-butyl-(R)-4-((1-(3-(2,6-bis(benzyloxy)pyridin-3-yl)-1-methyl-1H-indazol-7-yl)piperidin-4-yl)methyl)-3-(hydroxymethyl)piperazine-1-carboxylate

A stirred solution of 3-(2,6-bis(benzyloxy)pyridin-3-yl)-7-bromo-1-methyl-1H-indazole (1.40 g, 2.79 mmol), tert-butyl-(R)-3-(hydroxymethyl)-4-(piperidin-4-ylmethyl)piperazine-1-carboxylate (2.63 g, 8.39 mmol), and cesium carbonate (2.73 g, 8.39 mmol) in dioxane (14 mL) was degassed with argon for 5 minutes, followed by the addition of Pd-PEPPSI-iHeptCl (0.14 g, 0.14 mmol). The reaction mixture was degassed with argon for 5 minutes and heated to 100° C. for 18 h. The reaction mixture was filtered through a celite bed, concentrated under vacuum, and purified by column chromatography (SiO₂, 100-200 mesh; 30-50% ethyl acetate in petroleum ether as eluent) to afford tert-butyl-(R)-4-((1-(3-(2,6-bis(benzyloxy)pyridin-3-yl)-1-methyl-1H-indazol-7-yl)piperidin-4-yl)methyl)-3-(hydroxymethyl)piperazine-1-carboxylate (0.78 g) as an off-white solid.

LC-MS (ESI): m/z=733.79 [M+H]⁺.

### Preparation of tert-butyl-(3R)-4-((1-(3-(2,6-dioxopiperidin-3-yl)-1-methyl-1H-indazol-7-yl)piperidin-4-yl)methyl)-3-(hydroxymethyl)piperazine-1-carboxylate

To a solution of tert-butyl-(R)-4-((1-(3-(2,6-bis(benzyloxy)pyridin-3-yl)-1-methyl-1H-indazol-7-yl)piperidin-4-yl)methyl)-3-(hydroxymethyl)piperazine-1-carboxylate (0.78 g, 1.06 mmol) in THF (47.0 mL) was added acetic acid (0.78 mL) and 20% Pd(OH)₂ on carbon (1.05 g, 7.45 mmol). The reaction mixture was stirred under hydrogen atmosphere (80 psi) at room temperature for 18 h. The reaction mixture was filtered through a celite bed and washed with 40% THF in DCM (800 mL). The collected filtrate was concentrated under reduced pressure and triturated with n-pentane (30 mL) to afford tert-butyl-(3R)-4-((1-(3-(2,6-dioxopiperidin-3-yl)-1-methyl-1H-indazol-7-yl)piperidin-4-yl)methyl)-3-(hydroxymethyl)piperazine-1-carboxylate (0.70 g) as a brown solid.

LC-MS (ESI): m/z=555.67 [M+H]⁺

### Preparation of 3-(7-(4-(((R)-2-(hydroxymethyl)piperazin-1-yl)methyl)piperidin-1-yl)-1-methyl-1H-indazol-3-yl)piperidine-2,6-dione

To a solution of tert-butyl-(3R)-4-((1-(3-(2,6-dioxopiperidin-3-yl)-1-methyl-1H-indazol-7-yl)piperidin-4-yl)methyl)-3-(hydroxymethyl)piperazine-1-carboxylate (0.35 g, 0.63 mmol) in DCM (7.0 mL) was added TFA (3.5 mL) at 0° C. The reaction mixture was stirred at room temperature for 1 h. The reaction mixture was then concentrated under reduced pressure and triturated with n-pentane (10 mL) to afford 3-(7-(4-(((R)-2-(hydroxymethyl)piperazin-1-yl)methyl)piperidin-1-yl)-1-methyl-1H-indazol-3-yl)piperidine-2,6-dione (0.40 g) as a brown solid.

LC-MS (ESI): m/z=455.61 [M+H]⁺

### Preparation of 2-((6-((5-chloro-2-((3R)-4-((1-(3-(2,6-dioxopiperidin-3-yl)-1-methyl-1H-indazol-7-yl)piperidin-4-yl)methyl)-3-(hydroxymethyl)piperazin-1-yl)pyrimidin-4-yl)amino)-1-isopropyl-2-oxo-1,2-dihydro-1,8-naphthyridin-3-yl)oxy)-N-methylacetamide

To a stirred solution of 3-(7-(4-(((R)-2-(hydroxymethyl)piperazin-1-yl)methyl)piperidin-1-yl)-1-methyl-1H-indazol-3-yl)piperidine-2,6-dione (0.40 g, 0.88 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.19 g, 0.44 mmol) in DMSO (2.4 mL) was added DIPEA (2.4 mL). The reaction mixture was heated to 100° C. for 18 h. The reaction mixture was concentrated under reduced pressure and purified by prep-HPLC to afford 2-((6-((5-chloro-2-((3R)-4-((1-(3-(2,6-dioxopiperidin-3-yl)-1-methyl-1H-indazol-7-yl)piperidin-4-yl)methyl)-3-(hydroxymethyl)piperazin-1-yl)pyrimidin-4-yl)amino)-1-isopropyl-2-oxo-1,2-dihydro-1,8-naphthyridin-3-yl)oxy)-N-methylacetamide (0.129 g) as an off white solid.

¹H NMR (400 MHz, DMSO-d₆): δ 10.89 (s, 1H), 9.05 (s, 1H), 8.65 (d, J=2.40 Hz, 1H), 8.48 (br s, 1H), 8.09 (s, 1H), 7.92 (d, J=4.40 Hz, 1H), 7.36 (d, J=3.60 Hz, 1H), 7.16 (s, 1H), 7.01 (d, J=3.60 Hz, 2H), 5.95 (d, J=4.00 Hz, 1H), 4.62-4.53 (m, 3H), 4.44-4.40 (m, 1H), 4.24 (s, 3H), 4.11-4.06 (m, 1H), 3.99-3.89 (m, 1H), 3.68-3.63 (m, 1H), 3.22-3.16 (m, 5H), 2.85-2.80 (m, 1H), 2.67 (d, J=5.20 Hz, 3H), 2.65-2.64 (m, 4H), 2.62-2.61 (m, 1H), 2.43-2.39 (m, 6H), 1.92-1.90 (m, 1H), 1.89-1.87 (m, 1H), 1.78-1.75 (m, 1H), 1.57 (d, J=6.80 Hz, 6H), 1.38-1.36 (m, 1H).

LC-MS (ESI): m/z=855.56 [M+H]⁺.

### Example 44: 2-((6-((5-chloro-2-((3R)-4-((1-(3-(2,6-dioxopiperidin-3-yl)-1-methyl-1H-indazol-6-yl)piperidin-4-yl)methyl)-3-(hydroxymethyl)piperazin-1-yl)pyrimidin-4-yl)amino)-1-isopropyl-2-oxo-1,2-dihydro-1,8-naphthyridin-3-yl)oxy)-N-methylacetamide (Compound 153a)

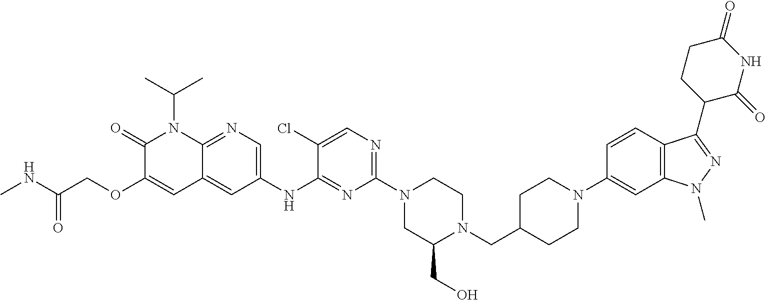 [SMILES: CNC(=O)COc1cc2cc(Nc3nc(N4CCN(CC5CCN(c6ccc7c(C8CCC(=O)NC8=O)nn(C)c7c6)CC5)[C@@H](CO)C4)ncc3Cl)cnc2n(C(C)C)c1=O]

### Preparation of tert-butyl-(R)-4-((1-(3-(2,6-bis(benzyloxy)pyridin-3-yl)-1-methyl-1H-indazol-6-yl)piperidin-4-yl)methyl)-3-(hydroxymethyl)piperazine-1-carboxylate

To a stirred solution of 3-(2,6-bis(benzyloxy)pyridin-3-yl)-6-bromo-1-methyl-1H-indazole (1.20 g, 2.40 mmol) and tert-butyl-(R)-3-(hydroxymethyl)-4-(piperidin-4-ylmethyl)piperazine-1-carboxylate (2.25 g, 7.19 mmol), and NaOtBu (0.69 g, 7.19 mmol) in dioxane (12 mL) was added Brettphos Pd G3 (0.22 g, 0.24 mmol). The resulting reaction was stirred and heated at 100° C. for 18 h. The reaction mixture was filtered through a bed of celite, and the filter cake was washed with DCM (200 mL). The filtrate was concentrated under vacuum and purified by flash silica gel column chromatography (75%-80% of ethyl acetate in petroleum ether) to afford tert-butyl-(R)-4-((1-(3-(2,6-bis(benzyloxy)pyridin-3-yl)-1-methyl-1H-indazol-6-yl)piperidin-4-yl)methyl)-3-(hydroxymethyl)piperazine-1-carboxylate (0.35 g) as a pale yellow semi-solid.

LC-MS (ESI): m/z=733.49 [M+H]⁺

### Preparation of tert-butyl-(3R)-4-((1-(3-(2,6-dioxopiperidin-3-yl)-1-methyl-1H-indazol-6-yl)piperidin-4-yl)methyl)-3-(hydroxymethyl)piperazine-1-carboxylate

To a stirred solution of tert-butyl-(R)-4-((1-(3-(2,6-bis(benzyloxy)pyridin-3-yl)-1-methyl-1H-indazol-6-yl)piperidin-4-yl)methyl)-3-(hydroxymethyl)piperazine-1-carboxylate (0.33 g, 0.45 mmol) and AcOH (0.99 mL) in THF (66.0 mL) was added 20% Pd(OH)₂/C (0.63 g, 4.50 mmol). The resulting mixture was then stirred under a hydrogen atmosphere (80 psi) at ambient temperature for 18 h. The reaction mixture was filtered through a bed of celite and washed with THF. The filtrated was concentrated under reduced pressure, washed with n-pentane, and dried to afford tert-butyl-(3R)-4-((1-(3-(2,6-dioxopiperidin-3-yl)-1-methyl-1H-indazol-6-yl)piperidin-4-yl)methyl)-3-(hydroxymethyl)piperazine-1-carboxylate (0.29 g) as a pale brown semi-solid.

LC-MS (ESI): m/z=555.67 (M+H)+

### Preparation of 3-(6-(4-(((R)-2-(hydroxymethyl)piperazin-1-yl)methyl)piperidin-1-yl)-1-methyl-1H-indazol-3-yl)piperidine-2,6-dione

To a solution of tert-butyl-(3R)-4-((1-(3-(2,6-dioxopiperidin-3-yl)-1-methyl-1H-indazol-6-yl)piperidin-4-yl)methyl)-3-(hydroxymethyl)piperazine-1-carboxylate (0.29 g, 0.52 mmol) in DCM (5.80 mL) was added TFA (1.45 mL). The reaction mixture was stirred at ambient temperature for 1 h. The reaction mixture was then concentrated under reduced pressure, triturated with n-pentane (20 mL), and dried to afford 3-(6-(4-(((R)-2-(hydroxymethyl)piperazin-1-yl)methyl)piperidin-1-yl)-1-methyl-1H-indazol-3-yl)piperidine-2,6-dione (0.39 g) as a pale brown semi-solid.

LC-MS (ESI): m/z=455.52 [M+H]⁺

### Preparation of 2-((6-((5-chloro-2-((3R)-4-((1-(3-(2,6-dioxopiperidin-3-yl)-1-methyl-1H-indazol-6-yl)piperidin-4-yl)methyl)-3-(hydroxymethyl)piperazin-1-yl)pyrimidin-4-yl)amino)-1-isopropyl-2-oxo-1,2-dihydro-1,8-naphthyridin-3-yl)oxy)-N-methylacetamide

To a stirred solution of 3-(6-(4-(((R)-2-(hydroxymethyl)piperazin-1-yl)methyl)piperidin-1-yl)-1-methyl-1H-indazol-3-yl)piperidine-2,6-dione (0.14 g, 0.31 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.07 g, 0.15 mmol) in DMSO (1.40 mL) was added DIPEA (1.40 mL). The mixture was then heated at 100° C. for 18 h. The reaction mixture was then purified by prep-HPLC to afford 2-((6-((5-chloro-2-((3R)-4-((1-(3-(2,6-dioxopiperidin-3-yl)-1-methyl-1H-indazol-6-yl)piperidin-4-yl)methyl)-3-(hydroxymethyl)piperazin-1-yl)pyrimidin-4-yl)amino)-1-isopropyl-2-oxo-1,2-dihydro-1,8-naphthyridin-3-yl)oxy)-N-methylacetamide (0.045 g) as an off-white solid. ¹H NMR (400 MHz, DMSO-d₆): δ 10.84 (s, 1H), 9.05 (s, 1H), 8.65 (d, J=2.00 Hz, 1H), 8.09 (s, 1H), 7.92 (d, J=4.40 Hz, 1H), 7.47 (d, J=8.80 Hz, 1H), 7.15 (s, 1H), 6.90 (d, J=8.80 Hz, 1H), 6.82 (s, 1H), 5.98 (br s, 1H), 4.61-0.00 (m, 3H), 4.23-4.24 (m, 1H), 4.05-4.02 (m, 1H), 3.84 (s, 3H), 3.77-3.79 (m, 3H), 3.65-3.67 (m, 1H), 3.32 (s, 3H), 2.84-2.82 (m, 1H), 2.72-2.73 (m, 5H), 2.67 (d, J=4.40 Hz, 3H), 2.50-2.50 (m, 1H), 2.37-2.35 (m, 2H), 2.16-2.15 (m, 2H), 1.92-1.86 (m, 1H), 1.81-1.75 (m, 2H), 1.56 (d, J=6.80 Hz, 6H), 1.28-1.24 (m, 3H).

LC-MS (ESI): m/z=855.56 [M+H]⁺

### Example 45: 2-((6-((5-chloro-2-((3S)-4-((1-(3-(2,6-dioxopiperidin-3-yl)-1-methyl-1H-indazol-6-yl)piperidin-4-yl)methyl)-3-(hydroxymethyl)piperazin-1-yl)pyrimidin-4-yl)amino)-1-isopropyl-2-oxo-1,2-dihydro-1,8-naphthyridin-3-yl)oxy)-N-methylacetamide (Compound 153b)

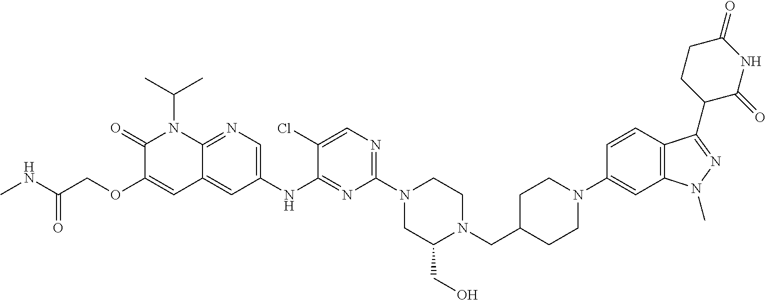 [SMILES: CNC(=O)COc1cc2cc(Nc3nc(N4CCN(CC5CCN(c6ccc7c(C8CCC(=O)NC8=O)nn(C)c7c6)CC5)[C@H](CO)C4)ncc3Cl)cnc2n(C(C)C)c1=O]

### Preparation of tert-butyl-(S)-4-((1-(3-(2,6-bis(benzyloxy)pyridin-3-yl)-1-methyl-1H-indazol-6-yl)piperidin-4-yl)methyl)-3-(hydroxymethyl)piperazine-1-carboxylate

To stirred solution of 3-(2,6-bis(benzyloxy)pyridin-3-yl)-6-bromo-1-methyl-1H-indazole (0.5 g, 0.99 mmol) and tert-butyl-(S)-3-(hydroxymethyl)-4-(piperidin-4-ylmethyl)piperazine-1-carboxylate (0.49 g, 1.49 mmol) in 1,4-dioxane (10.0 mL) was added cesium carbonate (0.96 g, 2.97 mmol). The reaction mixture was degassed for 10 minutes. Afterwards, Ruphos (0.05 g, 0.09 mmol) and Ruphos-PdG₃ (0.04 g, 0.05 mmol) were added, and the resulting solution was stirred at 100° C. for 5 h. The reaction mixture was then cooled to room temperature, filtered through a celite bed, washed with ethyl acetate (100 mL), and concentrated under reduced pressure to obtain a residue which was purified by flash column chromatography (SiO₂, 230-400 mesh, 25% ethyl acetate in petroleum ether) to afford tert-butyl-(S)-4-((1-(3-(2,6-bis(benzyloxy)pyridin-3-yl)-1-methyl-1H-indazol-6-yl)piperidin-4-yl)methyl)-3-(hydroxymethyl)piperazine-1-carboxylate as a brown semi-solid (0.3 g).

LC-MS (ESI): m/z=733.46 [M+H]⁺

### Preparation of tert-butyl-(3S)-4-((1-(3-(2,6-dioxopiperidin-3-yl)-1-methyl-1H-indazol-6-yl)piperidin-4-yl)methyl)-3-(hydroxymethyl)piperazine-1-carboxylate

To a solution of tert-butyl-(S)-4-((1-(3-(2,6-bis(benzyloxy)pyridin-3-yl)-1-methyl-1H-indazol-6-yl)piperidin-4-yl)methyl)-3-(hydroxymethyl)piperazine-1-carboxylate (0.30 g, 0.41 mmol) in THF (12.0 mL) and AcOH (0.3 mL) was added 20% Pd(OH)₂, (0.3 g, 100% w/w). The reaction mixture was stirred under hydrogen atmosphere (60 psi) for 16 h at room temperature. The reaction mixture was diluted with THE, filtered through a pad of celite, and washed with 100 mL of 20% THF in DCM. The filtrate was collected and concentrated to afford tert-butyl-(3S)-4-((1-(3-(2,6-dioxopiperidin-3-yl)-1-methyl-1H-indazol-6-yl)piperidin-4-yl)methyl)-3-(hydroxymethyl)piperazine-1-carboxylate as a brown semi-solid (0.24 g) which was used in the next step without further purification.

LC-MS (ESI): m/z=555.57 [M+H]⁺.

### Preparation of 3-(6-(4-(((S)-2-(hydroxymethyl)piperazin-1-yl)methyl)piperidin-1-yl)-1-methyl-1H-indazol-3-yl)piperidine-2,6-dione

To a solution of tert-butyl-(3S)-4-((1-(3-(2,6-dioxopiperidin-3-yl)-1-methyl-1H-indazol-6-yl)piperidin-4-yl)methyl)-3-(hydroxymethyl)piperazine-1-carboxylate (0.24 g, 0.43 mmol) in DCM (3.0 mL) was added TFA (1.2 mL). The reaction mixture was stirred for 3 h at room temperature. The reaction mixture was then concentrated and triturated with diethyl ether to obtain 3-(6-(4-(((S)-2-(hydroxymethyl)piperazin-1-yl)methyl)piperidin-1-yl)-1-methyl-1H-indazol-3-yl)piperidine-2,6-dione (0.22 g) as a TFA salt which was used in the next step without further purification.

LC-MS (ESI): m/z=455.27 [M+H]⁺.

### Preparation of 2-((6-((5-chloro-2-((3S)-4-((1-(3-(2,6-dioxopiperidin-3-yl)-1-methyl-1H-indazol-6-yl)piperidin-4-yl)methyl)-3-(hydroxymethyl)piperazin-1-yl)pyrimidin-4-yl)amino)-1-isopropyl-2-oxo-1,2-dihydro-1,8-naphthyridin-3-yl)oxy)-N-methylacetamide

To a stirred solution 3-(6-(4-(((S)-2-(hydroxymethyl)piperazin-1-yl)methyl)piperidin-1-yl)-1-methyl-1H-indazol-3-yl)piperidine-2,6-dione (0.11 g, 0.19 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.03 g, 0.08 mmol) in DMSO (2.0 mL) was added DIPEA (0.3 mL, 1.52 mmol). The reaction mixture was heated at 100° C. for 5 h. The reaction mixture was quenched with ice-cold water, and the solid thus formed was filtered and purified by prep-HPLC to afford 2-((6-((5-chloro-2-((3S)-4-((1-(3-(2,6-dioxopiperidin-3-yl)-1-methyl-1H-indazol-6-yl)piperidin-4-yl)methyl)-3-(hydroxymethyl)piperazin-1-yl)pyrimidin-4-yl)amino)-1-isopropyl-2-oxo-1,2-dihydro-1,8-naphthyridin-3-yl)oxy)-N-methylacetamide as an off-white solid (0.023 g).

¹H NMR 400 MHz, DMSO-d6: 10.85 (s, 1H), δ 9.05 (s, 1H), 8.65 (d, J=2.00 Hz, 1H), 8.09 (s, 1H), 7.92 (d, J=4.80 Hz, 1H), 7.47 (d, J=8.6 Hz, 1H), 7.15 (s, 1H), 6.90 (d, J=8.80 Hz, 1H), 6.82 (s, 1H), 5.96 (br s, 1H), 4.56 (s, 3H), 4.55 (br, 2H), 4.27-4.23 (m, 1H), 3.88 (s, 3H), 3.78 (d, J=11.60 Hz, 2H), 3.65-3.64 (m, 1H), 3.28 (br, 3H), 2.68-2.67 (m, 1H), 2.64-2.60 (m, 5H), 2.59-2.58 (m, 5H), 2.37 (br, 1H), 2.27-2.25 (m, 4H), 1.89-1.86 (m, 1H), 1.76-1.75 (m, 2H), 1.86-1.87 (m, 1H) 1.56 (d, J=6.80 Hz, 6H).

LC-MS (ESI): m/z=855.51 [M+H]⁺.

### Example 46: 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-(2-methoxyethyl)-2-oxo-1,2-dihydro-1,8-naphthyridin-3-yl)oxy)-N-methylacetamide (Compound 154)

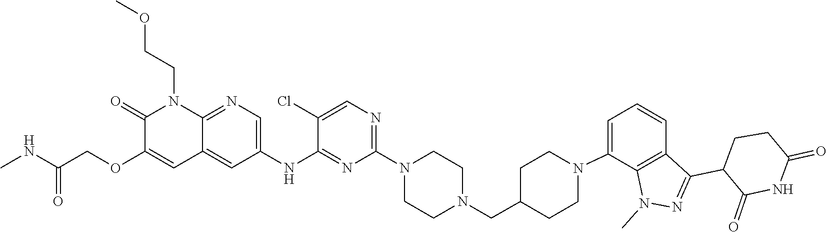 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)cnc2n(CCOC)c1=O]

### Preparation of 1-(2-methoxyethyl)-5-nitro-1H-pyrrolo[2,3-b]pyridine

To a stirred solution of 5-nitro-1H-pyrrolo[2,3-b]pyridine (20.0 g, 0.13 mol) and potassium carbonate (44.16 g, 0.32 mol) in DMF (400 mL) was added 1-bromo-2-methoxyethane (44.85 g, 0.32 mol) dropwise over a period of 20 min. The reaction was stirred at room temperature for 16 h. The reaction mixture was added slowly to ice-cold water, and the precipitate thus formed was filtered and dried under vacuum to afford 1-(2-methoxyethyl)-5-nitro-1H-pyrrolo[2,3-b]pyridine (20 g) as an off-white solid which was used in the next step without further purification.

LC-MS (ESI): m/z=222.03 [M+H]⁺

### Preparation of 1-(2-methoxyethyl)-5-nitro-1H-pyrrolo[2,3-b]pyridine-2,3-dione

To a stirred solution of 1-(2-methoxyethyl)-5-nitro-1H-pyrrolo[2,3-b]pyridine (20.0 g, 0.09 mol) in acetone (170 mL) was added acetic acid (640 mL) and H₂O (200 mL) at 0° C. Then, CrO₃ (117.16 g, 1.17 mol) was added slowly at 0° C. The reaction mixture was stirred at room temperature for 30 min. The reaction mixture was diluted with water (500 mL) and extracted with DCM (500 mL×4). The organic layers were separated and washed with brine (200 mL), dried over Na₂SO₄, filtered, and concentrated to afford 1-(2-methoxyethyl)-5-nitro-1H-pyrrolo[2,3-b]pyridine-2,3-dione (20 g) as a pale yellow solid which was used in the next step without further purification.

### Preparation of 3-hydroxy-1-(2-methoxyethyl)-6-nitro-1,8-naphthyridin-2 (1H)-one

To a cooled solution of 1-(2-methoxyethyl)-5-nitro-1H-pyrrolo[2,3-b]pyridine-2,3-dione (20.0 g, 0.08 mol) in EtOH (400 mL) was added triethylamine (23.37 mL, 0.17 mol). Trimethylsilyl diazomethane (2.0 M in diethylether, 16.8 mL, 0.17 mol) was added at 0° C. The reaction mixture was stirred at room temperature for 4 h. The reaction mixture was diluted with water (300 mL) and extracted with ethyl acetate (300 mL×3). The organic layers were separated and washed with brine, dried over anhydrous Na₂SO₄, and concentrated to afford 3-hydroxy-1-(2-methoxyethyl)-6-nitro-1,8-naphthyridin-2 (1H)-one (8 g) as a brown solid which was used in the next step without purification.

LC-MS (ESI): m/z=266.03 [M+H]⁺

### Preparation of 2-((1-(2-methoxyethyl)-6-nitro-2-oxo-1,2-dihydro-1,8-naphthyridin-3-yl)oxy)-N-methylacetamide

To a stirred solution of 3-hydroxy-1-(2-methoxyethyl)-6-nitro-1,8-naphthyridin-2 (1H)-one (8.0 g, 0.03 mol) in DMF (160 mL) was added cesium carbonate (9.77 g, 0.03 mol) and 2-bromo-N-methylacetamide (4.56 g, 0.03 mol) at room temperature under N₂ atmosphere. The reaction mixture was stirred at room temperature for 2 h. The reaction was quenched with cold water (500 mL) and extracted with 10% MeOH in DCM (300 mL×3). The organic layer was separated and washed with brine, dried over anhydrous Na₂SO₄, and concentrated to provide a crude product which was purified by column chromatography (SiO₂, 100-200 mesh; 0-100% ethyl acetate in petroleum ether) to afford 2-((1-(2-methoxyethyl)-6-nitro-2-oxo-1,2-dihydro-1,8-naphthyridin-3-yl)oxy)-N-methylacetamide (2.8 g) as an off white solid.

LC-MS (ESI): m/z=337.37 [M+H]⁺

### Preparation of 2-((6-amino-1-(2-methoxyethyl)-2-oxo-1,2-dihydro-1,8-naphthyridin-3-yl)oxy)-N-methylacetamide

To a stirred solution of 2-((1-(2-methoxyethyl)-6-nitro-2-oxo-1,2-dihydro-1,8-naphthyridin-3-yl)oxy)-N-methylacetamide (2.8 g, 0.008 mol) in MeOH (56 mL) was added Fe powder (4.46 g, 0.08 mol) and NH₄Cl (4.27 g, 0.08 mol). The reaction mixture was refluxed and allowed to stir for 6 h at 65° C. The reaction mixture was filtered through a celite pad and concentrated under reduced pressure to afford 2-((6-amino-1-(2-methoxyethyl)-2-oxo-1,2-dihydro-1,8-naphthyridin-3-yl)oxy)-N-methylacetamide (0.8 g) as an off-light yellow solid which was used in the next step without purification.

LC-MS (ESI): m/z=[M+H]⁺

### Preparation of 2-((6-((2,5-dichloropyrimidin-4-yl)amino)-1-(2-methoxyethyl)-2-oxo-1,2-dihydro-1,8-naphthyridin-3-yl)oxy)-N-methylacetamide

To a stirred solution of 2-((6-amino-1-(2-methoxyethyl)-2-oxo-1,2-dihydro-1,8-naphthyridin-3-yl)oxy)-N-methylacetamide (1.0 g, 3.81 mmol) in DMSO (4 mL) were added N,N-diisopropylethylamine (0.92 mL, 2.2 mol) and 2,4,5-trichloropyrimidine (0.23 g, 1.30 mmol). The reaction mixture was stirred at room temperature for 4 h. The reaction was quenched with ice water and stirred for 20 min. The precipitate thus formed was filtered, dried under vacuum, and purified by column chromatography (SiO₂, 100-200 mesh; 0-100% ethyl acetate in petroleum ether) to afford 2-((6-((2,5-dichloropyrimidin-4-yl)amino)-1-(2-methoxyethyl)-2-oxo-1,2-dihydro-1,8-naphthyridin-3-yl)oxy)-N-methylacetamide (0.15 g) as an off white solid.

LC-MS (ESI): m/z=453.11 [M+H]⁺.

### Preparation of 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-(2-methoxyethyl)-2-oxo-1,2-dihydro-1,8-naphthyridin-3-yl)oxy)-N-methylacetamide

To a stirred solution of 2-((6-((2,5-dichloropyrimidin-4-yl)amino)-1-(2-methoxyethyl)-2-oxo-1,2-dihydro-1,8-naphthyridin-3-yl)oxy)-N-methylacetamide (0.15 g, 0.33 mmol) in DMSO (3 mL) was added N,N-diisopropylethylamine (0.46 mL, 2.64 mol) and 3-(1-methyl-7-(4-(piperazin-1-ylmethyl)piperidin-1-yl)-1H-indazol-3-yl)piperidine-2,6-dione (0.28 g, 0.66 mmol). The reaction mixture was stirred at 120° C. for 16 h. The reaction was concentrated under reduced pressure and purified by prep-HPLC to afford 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-(2-methoxyethyl)-2-oxo-1,2-dihydro-1,8-naphthyridin-3-yl)oxy)-N-methylacetamide (0.065 g) as an off white solid.

¹H NMR (400 MHz, DMSO-d₆): δ 10.87 (s, 1H), 9.10 (s, 1H), 8.67 (s, 1H), 8.31 (s, 1H), 8.09 (s, 1H), 7.98 (s, 1H), 7.36-7.34 (m, 2H), 7.18 (s, 1H), 6.99 (br s, 2H), 4.75-4.65 (m, 4H), 4.45-4.30 (m, 1H), 4.23 (s, 3H), 3.70-3.55 (m, 6H), 3.33-3.00 (m, 4H), 2.70-2.50 (m, 9H), 2.40-2.10 (m, 4H), 1.89 (d, 2H), 1.50-1.30 (m, 2H), 1.23 (s, 2H), 1.09 (s, 1H).

LC-MS (ESI): m/z=841.43 [M+H]⁺.

### Example 47: 2-((6-((5-chloro-2-((2S)-4-((1-(3-(2,6-dioxopiperidin-3-yl)-1-methyl-1H-indazol-7-yl)piperidin-4-yl)methyl)-2-(hydroxymethyl)piperazin-1-yl)pyrimidin-4-yl)amino)-1-isopropyl-2-oxo-1,2-dihydro-1,8-naphthyridin-3-yl)oxy)-N-methylacetamide (Compound 155a)

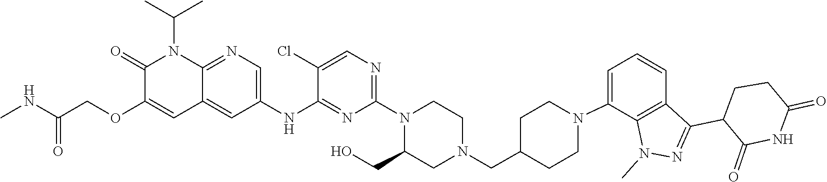 [SMILES: CNC(=O)COc1cc2cc(Nc3nc(N4CCN(CC5CCN(c6cccc7c(C8CCC(=O)NC8=O)nn(C)c67)CC5)C[C@H]4CO)ncc3Cl)cnc2n(C(C)C)c1=O]

### Preparation of tert-butyl-(S)-4-((1-((benzyloxy) carbonyl)piperidin-4-yl)methyl)-2-(hydroxymethyl)piperazine-1-carboxylate

To a solution of tert-butyl-(S)-2-(hydroxymethyl)piperazine-1-carboxylate (2.0 g, 9.24 mmol), benzyl-4-formylpiperidine-1-carboxylate (2.28 g, 9.24 mmol), and NaBH(OAc)₃ (2.35 g, 11.09 mmol) in DCM (40 mL) was added acetic acid (0.5 mL, 9.24 mmol). The reaction mixture was stirred at room temperature for 16 h. The reaction mixture was quenched with ice-cold water (100 mL) and extracted with DCM (3×100 mL). The combined organic layers were washed with brine (100 mL), dried over anhydrous Na₂SO₄, filtered, dried under vacuum and purified by column chromatography (SiO₂; 40% ethyl acetate in petroleum ether) to afford tert-butyl-(S)-4-((1-((benzyloxy) carbonyl)piperidin-4-yl)methyl)-2-(hydroxymethyl)piperazine-1-carboxylate (4.0 g) as a white solid.

LC-MS (ESI): m/z=448.32 [M+H]⁺

### Preparation of tert-butyl-(S)-2-(hydroxymethyl)-4-(piperidin-4-ylmethyl)piperazine-1-carboxylate

To a solution of tert-butyl-(S)-4-((1-((benzyloxy) carbonyl)piperidin-4-yl)methyl)-2-(hydroxymethyl)piperazine-1-carboxylate (4.0 g, 8.93 mmol) in THF (240 mL) was added 10% palladium on carbon (4.0 g, 100% w/w). The reaction mixture was stirred under hydrogen atmosphere (60 psi) at room temperature for 16 h. The reaction mixture was filtered through a celite bed, concentrated, washed with n-pentane, and dried to afford tert-butyl-(S)-2-(hydroxymethyl)-4-(piperidin-4-ylmethyl)piperazine-1-carboxylate (2.5 g) as a white solid which was used in the next step without purification.

LC-MS (ESI): m/z=314.51 [M+H]⁺.

### Preparation of tert-butyl-(S)-4-((1-(3-(2,6-bis(benzyloxy)pyridin-3-yl)-1-methyl-1H-indazol-7-yl)piperidin-4-yl)methyl)-2-(hydroxymethyl)piperazine-1-carboxylate

A solution of 3-(2,6-bis(benzyloxy)pyridin-3-yl)-7-bromo-1-methyl-1H-indazole (0.90 g, 1.79 mmol), tert-butyl-(S)-2-(hydroxymethyl)-4-(piperidin-4-ylmethyl)piperazine-1-carboxylate (1.12 g, 3.59 mmol), and cesium carbonate (1.75 g, 5.39 mmol) in 1,4-dioxane (18 mL) was degassed with argon for 15 minutes. Afterwards, Pd-PEPPSI-IHeptCl (80 mg, 0.09 mmol) was added, and the solution was again degassed for 5 min. The reaction mixture was then stirred and heated at 100° C. for 16 h. The reaction mixture was quenched with ice-cold water (50 mL) and extracted with ethyl acetate (3×50 mL). The combined organic layers were washed with brine (50 mL), dried over anhydrous Na₂SO₄, filtered, dried under vacuum, and purified by column chromatography (SiO₂; 40% ethyl acetate in petroleum ether) to afford tert-butyl-(S)-4-((1-(3-(2,6-bis(benzyloxy)pyridin-3-yl)-1-methyl-1H-indazol-7-yl)piperidin-4-yl)methyl)-2-(hydroxymethyl)piperazine-1-carboxylate (0.71 g) as a pale brown solid.

LC-MS (ESI): m/z=733.7 [M+H]⁺

### Preparation of tert-butyl-(2S)-4-((1-(3-(2,6-dioxopiperidin-3-yl)-1-methyl-1H-indazol-7-yl)piperidin-4-yl)methyl)-2-(hydroxymethyl)piperazine-1-carboxylate

To a solution of tert-butyl-(S)-4-((1-(3-(2,6-bis(benzyloxy)pyridin-3-yl)-1-methyl-1H-indazol-7-yl)piperidin-4-yl)methyl)-2-(hydroxymethyl)piperazine-1-carboxylate (0.71 g, 0.96 mmol) in THF (28.0 mL) was added 20% Pd(OH)₂ on carbon (0.71 g, 100% w/w). The reaction mixture was stirred under hydrogen atmosphere (60 psi) at room temperature for 6 h. The reaction mixture was then filtered through a celite bed, concentrated under vacuum, washed with n-pentane, and dried to afford tert-butyl-(2S)-4-((1-(3-(2,6-dioxopiperidin-3-yl)-1-methyl-1H-indazol-7-yl)piperidin-4-yl)methyl)-2-(hydroxymethyl)piperazine-1-carboxylate (0.52 g) as a green solid.

LC-MS (ESI): m/z=555.67 [M+H]⁺.

### Preparation of 3-(7-(4-(((S)-3-(hydroxymethyl)piperazin-1-yl)methyl)piperidin-1-yl)-1-methyl-1H-indazol-3-yl)piperidine-2,6-dione

To a solution of tert-butyl-(2S)-4-((1-(3-(2,6-dioxopiperidin-3-yl)-1-methyl-1H-indazol-7-yl)piperidin-4-yl)methyl)-2-(hydroxymethyl)piperazine-1-carboxylate (0.52 g, 0.93 mmol) in DCM (5.2 mL) cooled to 0° C. was added TFA (2.6 mL). The reaction mixture was stirred at room temperature for 3 h. The reaction mixture was concentrated under vacuum and washed with n-pentane to afford 3-(7-(4-(((S)-3-(hydroxymethyl)piperazin-1-yl)methyl)piperidin-1-yl)-1-methyl-1H-indazol-3-yl)piperidine-2,6-dione (0.42 g) as a white solid.

LC-MS (ESI): m/z=455.56 [M+H]⁺

### Preparation of 2-((6-((5-chloro-2-((2S)-4-((1-(3-(2,6-dioxopiperidin-3-yl)-1-methyl-1H-indazol-7-yl)piperidin-4-yl)methyl)-2-(hydroxymethyl)piperazin-1-yl)pyrimidin-4-yl)amino)-1-isopropyl-2-oxo-1,2-dihydro-1,8-naphthyridin-3-yl)oxy)-N-methylacetamide

To a solution 3-(7-(4-(((S)-3-(hydroxymethyl)piperazin-1-yl)methyl)piperidin-1-yl)-1-methyl-1H-indazol-3-yl)piperidine-2,6-dione (0.2 g, 0.44 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.09 g, 0.22 mmol) in DMSO was added N,N-diisopropylethylamine (0.37 mL, 2.82 mmol) and stirred at 100° C. for 16 h. The reaction mixture was poured into ice cold water and stirred for 15 minutes. The resulting precipitate was filtered, dried, and purified by prep-HPLC to afford 2-((6-((5-chloro-2-((2S)-4-((1-(3-(2,6-dioxopiperidin-3-yl)-1-methyl-1H-indazol-7-yl)piperidin-4-yl)methyl)-2-(hydroxymethyl)piperazin-1-yl)pyrimidin-4-yl)amino)-1-isopropyl-2-oxo-1,2-dihydro-1,8-naphthyridin-3-yl)oxy)-N-methylacetamide (0.017 g) as an off white solid.

¹H NMR (400 MHz, DMSO-d₆): δ 10.87 (s, 1H), 9.07 (s, 1H), 8.63 (s, 1H), 8.48 (s, 1H), 8.09 (s, 1H), 7.93 (d, J=4.40 Hz, 1H), 7.37 (t, J=4.40 Hz, 1H), 7.21 (s, 1H), 7.01 (d, J=4.40 Hz, 2H), 5.96 (br s, 1H), 4.87-4.84 (m, 1H), 4.54 (s, 3H), 4.31-4.25 (m, 4H), 3.84 (br s, 1H), 3.50 (br, 1H), 3.20-3.03 (m, 5H), 2.88-2.86 (m, 1H), 2.69-2.57 (m, 7H), 2.19-2.16 (m, 3H), 1.98-1.92 (m, 4H), 1.80-1.70 (m, 1H), 1.5-1.6 (m, 6H), 1.3-1.5 (m, 2H), 1.35 (s, 1H).

LC-MS (ESI): m/z=855.56 [M+H]⁺

### Example 48: 2-((6-((5-chloro-2-((6S,7S)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6S,7S)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide] (Compound 156a)

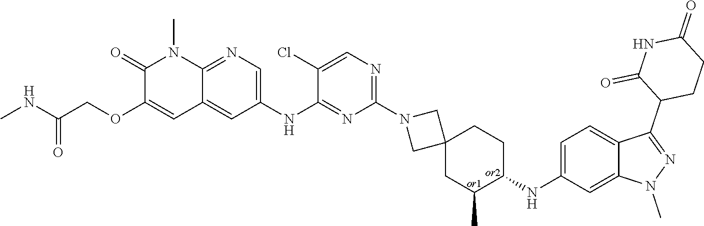 [SMILES: CNC(=O)COc1cc2cc(Nc3nc(N4CC5(CC[C@H](Nc6ccc7c(C8CCC(=O)NC8=O)nn(C)c7c6)[C@@H](C)C5)C4)ncc3Cl)cnc2n(C)c1=O]

### Preparation of tert-butyl-(6S,7S)-7-((3-(2,6-dioxopiperidin-3-yl)-1-methyl-1H-indazol-6-yl)amino)-6-methyl-2-azaspiro[3.5]nonane-2-carboxylate

To a solution of tert-butyl-(6S,7S)-7-((3-(2,6-bis(benzyloxy)pyridin-3-yl)-1-methyl-1H-indazol-6-yl)amino)-6-methyl-2-azaspiro[3.5]nonane-2-carboxylate (0.80 g, 1.18 mmol) in THF (80.0 mL) was added 20% Pd(OH)₂/C (0.80 g, 100% w/w). The reaction mixture was stirred under hydrogen atmosphere (85 psi) at room temperature for 16 h. The reaction mixture was diluted with THF (50 mL), filtered through a celite bed, and washed with THF:DCM (1:1, 200 mL). The filtrate was concentrated and dried to afford tert-butyl-(6S,7S)-7-((3-(2,6-dioxopiperidin-3-yl)-1-methyl-1H-indazol-6-yl)amino)-6-methyl-2-azaspiro[3.5]nonane-2-carboxylate (0.66 g) as a black solid which was used in the next step without further purification.

LC-MS (ESI): m/z=496.35 [M+H]⁺

### Preparation of 3-(1-methyl-6-(((6S,7S)-6-methyl-2-azaspiro[3.5]nonan-7-yl)amino)-1H-indazol-3-yl)piperidine-2,6-dione

To a solution of tert-butyl-(6S,7S)-7-((3-(2,6-dioxopiperidin-3-yl)-1-methyl-1H-indazol-6-yl)amino)-6-methyl-2-azaspiro[3.5]nonane-2-carboxylate (0.65 g, 1.31 mmol) in DCM (13.0 mL) was added TFA (6.5 mL, 10 V) at 0° C. The reaction mixture was stirred at room temperature for 3 h. The reaction mixture was concentrated under reduced pressure, co-distilled with DCM (2×15 mL), and triturated with diethyl ether (2×10 mL) to afford 3-(1-methyl-6-(((6S,7S)-6-methyl-2-azaspiro[3.5]nonan-7-yl)amino)-1H-indazol-3-yl)piperidine-2,6-dione as a brown semi-solid (0.60 g).

LC-MS (ESI): m/z=396.56 [M+H]⁺

### Preparation of 2-((6-((5-chloro-2-((6S,7S)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6S,7S)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide]

To a solution of 3-(1-methyl-6-(((6S,7S)-6-methyl-2-azaspiro[3.5]nonan-7-yl)amino)-1H-indazol-3-yl)piperidine-2,6-dione (0.30 g, 0.75 mmol) in DMSO (3.0 mL) was added DIPEA (3.0 mL) and 2-((6-((2,5-dichloropyrimidin-4-yl)amino)-1-methyl-2-oxo-1,2-dihydro-1,8-naphthyridin-3-yl)oxy)-N-methylacetamide (0.22 g, 0.53 mmol). The reaction mixture was stirred at 100° C. for 16 h. The reaction mixture was concentrated under reduced pressure and purified by prep-HPLC to afford 2-((6-((5-chloro-2-((6S,7S)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6S,7S)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide] (0.027 g) as a brown solid.

¹H NMR (400 MHz, DMSO-d₆): δ 10.81 (s, 1H), 9.04 (s, 1H), 8.84 (s, 1H), 8.55-8.48 (m, 1H), 8.07 (s, 1H), 7.98 (d, J=4.40 Hz, 1H), 7.29 (d, J=8.40 Hz, 1H), 7.20 (s, 1H), 6.50 (d, J=8.80 Hz, 1H), 6.36 (s, 1H), 5.65 (d, J=9.20 Hz, 1H), 4.65 (s, 1H), 4.16 (q, J=5.20 Hz, 1H), 3.79-3.75 (m, 8H), 3.67 (s, 2H), 3.00-2.98 (m, 1H), 2.67 (d, J=4.40 Hz, 3H), 2.57 (t, J=34.00 Hz, 2H), 2.26-2.22 (m, 1H), 2.16-2.15 (m, 1H), 2.07-1.89 (m, 4H), 1.68-1.62 (m, 3H), 1.52 (br, 1H), 1.39 (t, J=12.40 Hz, 1H), 0.97 (d, J=6.00 Hz, 3H).

LC-MS (ESI): m/z=768.51 [M+H]⁺.

### Example 49: 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-(cyclopropylmethyl)-2-oxo-1,2-dihydro-1,8-naphthyridin-3-yl)oxy)-N-methylacetamide (Compound 157)

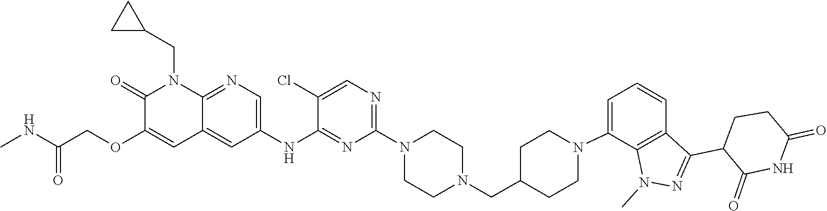 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)cnc2n(CC2CC2)c1=O]

### Preparation of 1-(cyclopropylmethyl)-5-nitro-1H-pyrrolo[2,3-b]pyridine

To a stirred solution of 5-nitro-1H-pyrrolo[2,3-b]pyridine (20.0 g, 122.60 mmol) and potassium carbonate (25.41 g, 183.90 mol) in dry DMF (400 mL) was added (bromomethyl)cyclopropane (41.06 g, 306.50 mmol) over 20 minutes. The reaction mixture was stirred at room temperature for 16 h. Ice-cold water (1000 mL) was added to the reaction mixture, and the precipitate thus formed was filtered and dried under vacuum to afford 1-(cyclopropylmethyl)-5-nitro-1H-pyrrolo[2,3-b]pyridine (19 g) as an off white solid which was used in the next reaction without further purification.

LC-MS (ESI): m/z=218.29 [M+H]⁺

### Preparation of 1-(cyclopropylmethyl)-5-nitro-1H-pyrrolo[2,3-b]pyridine-2,3-dione

To a stirred solution of 1-(cyclopropylmethyl)-5-nitro-1H-pyrrolo[2,3-b]pyridine (3.0 g, 13.81 mmol) in acetone (21 mL), acetic acid (96 mL), and H₂O (20 mL) at 10° C. was added CrO₃ (17.9 g, 179.64 mmol) over 5 min. The reaction mixture was stirred at same temperature for 0.5 h. The reaction mixture was diluted with water (50 mL) and extracted with DCM (50 mL×2). The organic layers were combined, washed with brine (60 mL×2), dried over Na₂SO₄, filtered, and concentrated to afford 1-(cyclopropylmethyl)-5-nitro-1H-pyrrolo[2,3-b]pyridine-2,3-dione (3.0 g) as a yellow solid which was used in the next step without further purification.

LC-MS (ESI): m/z=264.19 [M+NH_{3]}

### Preparation of 1-(cyclopropylmethyl)-3-methoxy-6-nitro-1,8-naphthyridin-2 (1H)-one

To a stirred solution of 1-(cyclopropylmethyl)-5-nitro-1H-pyrrolo[2,3-b]pyridine-2,3-dione (3.0 g, 11.94 mmol) in EtOH (40 mL) was added triethyl amine (6.71 mL, 47.79 mmol) and trimethylsilyl diazomethane (2.0 M in diethyl ether, 23.89 mL, 47.79 mmol) at 0° C. The reaction mixture was stirred at same temperature for 0.5 h. The reaction mixture was diluted with water (30 mL) and extracted with ethyl acetate (50 mL×3). The combined organic layer was washed with brine (20 mL), dried over Na₂SO₄, and concentrated to obtained crude product which was purified by flash column chromatography (SiO₂, 100-200 mesh, 40-50% ethyl acetate in petroleum Ether) to afford 1-(cyclopropylmethyl)-3-methoxy-6-nitro-1,8-naphthyridin-2 (1H)-one (0.7 g) as a yellow solid.

LC-MS (ESI): m/z=276.24 [M+H]⁺

### Preparation of 1-(cyclopropylmethyl)-3-hydroxy-6-nitro-1,8-naphthyridin-2 (1H)-one

To a stirred solution of 1-(cyclopropylmethyl)-3-methoxy-6-nitro-1,8-naphthyridin-2 (1H)-one (0.7 g, 5.88 mmol) in DCM (16 mL) was added BBr₃ (1M in DCM, 6.47 mL, 6.47 mmol) at 0° C. The reaction mixture was stirred at room temperature for 2.5 h. The reaction mixture was concentrated under vacuum to afford 1-(cyclopropylmethyl)-3-hydroxy-6-nitro-1,8-naphthyridin-2 (1H)-one (2.0 g) as a brown solid.

LC-MS (ESI): m/z=262.09 [M+H]

### Preparation of 2-((1-(cyclopropylmethyl)-6-nitro-2-oxo-1,2-dihydro-1,8-naphthyridin-3-yl)oxy)-N-methylacetamide

To a stirred solution of 1-(cyclopropylmethyl)-3-hydroxy-6-nitro-1,8-naphthyridin-2 (1H)-one (2.0 g, 7.66 mmol) in DMF (20 mL) was added cesium carbonate (2.98 g, 9.19 mmol) and 2-bromo-N-methylacetamide (1.39 g, 9.19 mmol) at 0° C. The reaction mixture was stirred at same temperature for 2.5 h. The reaction was quenched with cold water (50 mL) and extracted with 10% MeOH in DCM (30 mL×3). The combined organic layer was washed with brine (10 mL), dried over Na₂SO₄, and concentrated to afford a crude product which was purified by column chromatography (SiO₂, 100-200 mesh; 0-100% ethyl acetate in petroleum ether) to afford 2-((1-(cyclopropylmethyl)-6-nitro-2-oxo-1,2-dihydro-1,8-naphthyridin-3-yl)oxy)-N-methylacetamide (0.73 g) as an off white solid.

LC-MS (ESI): m/z=337.33 [M+H]⁺

### Preparation of 2-((6-amino-1-(cyclopropylmethyl)-2-oxo-1,2-dihydro-1,8-naphthyridin-3-yl)oxy)-N-methylacetamide

To a stirred solution of 2-((1-(cyclopropylmethyl)-6-nitro-2-oxo-1,2-dihydro-1,8-naphthyridin-3-yl)oxy)-N-methylacetamide (0.73 g, 2.19 mmol) in MeOH (14.5 mL) was added Fe (0.664 g, 21.90 mmol) and NH₄Cl (0.318 g, 10.99 mmol). The reaction mixture was heated to 70° C. for 4 h. After completion, the reaction mixture was cooled to room temperature and filtered through a celite pad (1.1 g). The filtrate was concentrated under reduced pressure to provide 2-((6-amino-1-(cyclopropylmethyl)-2-oxo-1,2-dihydro-1,8-naphthyridin-3-yl)oxy)-N-methylacetamide (0.4 g) as a brown solid

LC-MS (ESI): m/z=303.39 [M+H]⁺

### Preparation of 2-((1-(cyclopropylmethyl)-6-((2,5-dichloropyrimidin-4-yl)amino)-2-oxo-1,2-dihydro-1,8-naphthyridin-3-yl)oxy)-N-methylacetamide

To a stirred solution of 2-((6-amino-1-(cyclopropylmethyl)-2-oxo-1,2-dihydro-1,8-naphthyridin-3-yl)oxy)-N-methylacetamide (0.4 g, 1.32 mmol) in dry DMSO (4.0 mL) was added N, N-diisopropylethylamine (1.18 mL, 6.61 mmol) and 2,4,5-trichloropyrimidine (0.48 g, 2.64 mmol) at room temperature. The reaction of stirred for 4 h. The reaction was quenched with cold water (5 mL) and extracted with 2% MeOH in DCM (10 mL×3). The combined organic layer was washed with brine (7.0 mL), dried over Na₂SO₄, and concentrated to afford 2-((1-(cyclopropylmethyl)-6-((2,5-dichloropyrimidin-4-yl)amino)-2-oxo-1,2-dihydro-1,8-naphthyridin-3-yl)oxy)-N-methylacetamide (0.25 g) as a brown solid which was used in the next step without purification.

LC-MS (ESI): m/z=449.12 [M+H]⁺

### Preparation of 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-(cyclopropylmethyl)-2-oxo-1,2-dihydro-1,8-naphthyridin-3-yl)oxy)-N-methylacetamide

To a stirred solution of 2-((1-(cyclopropylmethyl)-6-((2,5-dichloropyrimidin-4-yl)amino)-2-oxo-1,2-dihydro-1,8-naphthyridin-3-yl)oxy)-N-methylacetamide (0.25 g, 0.54 mmol) in dry DMSO (2.5 mL) was added N, N-Diisopropylethylamine (2.5 mL, 0.27 mmol) and 3-(1-methyl-7-(4-(piperazin-1-ylmethyl)piperidin-1-yl)-1H-indazol-3-yl)piperidine-2,6-dione (0.12 g, 0.27 mmol) at room temperature. The reaction mixture was stirred and heated at 110° C. for 4 h. The reaction mixture was cooled to rt, concentrated under reduced pressure, and purified by prep-HPLC to afford 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-(cyclopropylmethyl)-2-oxo-1,2-dihydro-1,8-naphthyridin-3-yl)oxy)-N-methylacetamide (0.018 g) as an off white solid.

LC-MS (ESI): m/z=837.58 [M+H]⁺

¹H NMR data: (400 MHz, DMSO-d₆): δ 10.88 (s, 1H), 9.27-9.22 (m, 2H), 8.67 (d, J=2.40 Hz, 1H), 8.28 (d, J=2.40 Hz, 1H), 8.19 (s, 1H), 7.98 (d, J=4.80 Hz, 1H), 7.02-6.95 (m, 4H), 4.62 (s, 2H), 4.33-4.32 (m, 5H), 4.25 (s, 3H), 3.58 (d, J=10.40 Hz, 2H), 3.08-3.05 (m, 8H), 2.63-2.61 (m, 7H), 2.31-2.29 (m, 1H), 2.18-2.16 (m, 1H), 1.91-1.89 (m, 1H), 1.53-1.50 (m, 2H), 1.34-1.32 (m, 1H), 1.24-1.23 (m, 1H), 0.44-0.43 (m, 4H).

### Example 50: 2-((6-((5-chloro-2-(4-(2-(1-(3-(2,6-dioxopiperidin-3-yl)-1-methyl-1H-indazol-6-yl)piperidin-4-yl) propan-2-yl)piperazin-1-yl)pyrimidin-4-yl)amino)-1-isopropyl-2-oxo-1,2-dihydro-1,8-naphthyridin-3-yl)oxy)-N-methylacetamide (Compound 158)

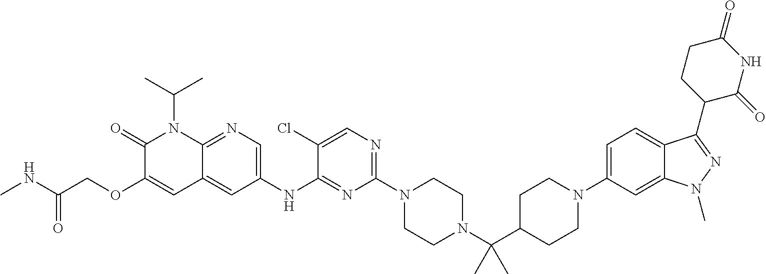 [SMILES: CNC(=O)COc1cc2cc(Nc3nc(N4CCN(C(C)(C)C5CCN(c6ccc7c(C8CCC(=O)NC8=O)nn(C)c7c6)CC5)CC4)ncc3Cl)cnc2n(C(C)C)c1=O]

### Preparation of tert-butyl 4-(2-(1-(3-(2,6-bis(benzyloxy)pyridin-3-yl)-1-methyl-1H-indazol-6-yl)piperidin-4-yl) propan-2-yl)piperazine-1-carboxylate

To a stirred solution of tert-butyl 4-(2-(piperidin-4-yl) propan-2-yl)piperazine-1-carboxylate (1 g, 3.21 mmol) and 3-(2,6-bis(benzyloxy)pyridin-3-yl)-6-bromo-1-methyl-1H-indazole (0.80 g, 1.60 mmol) in 1,4-dioxane (10 mL) at room temperature under N₂ atmosphere was added ^{t}BuONa (0.62 g, 6.42 mmol) and Ruphos-Pd-G3 (0.26 g, 0.32 mmol). The reaction mixture was stirred and heated at 100° C. for 16 h. After completion, the reaction mixture was quenched with ice cold water (20 mL) and extracted with ethyl acetate (2×100 mL). The organic layer was washed with brine (50 mL), dried over anhydrous Na₂SO₄, filtered, and concentrated to provide a crude product which was purified by flash column chromatography (SiO₂, 60-120, 50-60% ethyl acetate in petroleum ether) to afford tert-butyl 4-(2-(1-(3-(2,6-bis(benzyloxy)pyridin-3-yl)-1-methyl-1H-indazol-6-yl)piperidin-4-yl) propan-2-yl)piperazine-1-carboxylate (0.73 g).

LC-MS (ESI): m/z=731.77 [M+H]⁺

### Preparation of tert-butyl 4-(2-(1-(3-(2,6-dioxopiperidin-3-yl)-1-methyl-1H-indazol-6-yl)piperidin-4-yl) propan-2-yl)piperazine-1-carboxylate

To a stirred solution of tert-butyl 4-(2-(1-(3-(2,6-bis(benzyloxy)pyridin-3-yl)-1-methyl-1H-indazol-6-yl)piperidin-4-yl) propan-2-yl)piperazine-1-carboxylate (0.70 g, 0.95 mmol) in THF (14 mL) at room temperature under N₂ atmosphere was added palladium on activated carbon (10%) (0.70 g, 100% w/w). The reaction mixture was stirred at room temperature for 16 h with a hydrogen balloon (30 psi). After completion, the reaction mixture was filtered through a celite bed, and the filtrate was concentrated under reduced pressure to afford tert-butyl 4-(2-(1-(3-(2,6-dioxopiperidin-3-yl)-1-methyl-1H-indazol-6-yl)piperidin-4-yl) propan-2-yl)piperazine-1-carboxylate (0.37 g) which was used in the next step without further purification.

LC-MS (ESI): m/z=553.69 [M+H]⁺

### Preparation of 3-(1-methyl-6-(4-(2-(piperazin-1-yl) propan-2-yl)piperidin-1-yl)-1H-indazol-3-yl)piperidine-2,6-dione

To a stirred solution of tert-butyl 4-(2-(1-(3-(2,6-dioxopiperidin-3-yl)-1-methyl-1H-indazol-6-yl)piperidin-4-yl) propan-2-yl)piperazine-1-carboxylate (0.4 g, 0.74 mmol) in DCM (4.0 mL) was added TFA (2.0 mL) at 0° C. under N₂ atmosphere. The reaction mixture was stirred at room temperature for 3 h. The reaction mixture was then concentrated under reduced pressure, co-distilled with DCM, and triturated with diethyl ether (2×20 mL) to afford 3-(1-methyl-6-(4-(2-(piperazin-1-yl) propan-2-yl)piperidin-1-yl)-1H-indazol-3-yl)piperidine-2,6-dione (0.30 g).

LC-MS (ESI): m/z=453.59 [M+H]

### Preparation of 2-((6-((5-chloro-2-(4-(2-(1-(3-(2,6-dioxopiperidin-3-yl)-1-methyl-1H-indazol-6-yl)piperidin-4-yl) propan-2-yl)piperazin-1-yl)pyrimidin-4-yl)amino)-1-isopropyl-2-oxo-1,2-dihydro-1,8-naphthyridin-3-yl)oxy)-N-methylacetamide

To a stirred solution of 3-(1-methyl-6-(4-(2-(piperazin-1-yl) propan-2-yl)piperidin-1-yl)-1H-indazol-3-yl)piperidine-2,6-dione (0.150 g, 0.33 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.069 g, 0.16 mmol) in DMSO (3 mL) was added N,N-diisopropylethylamine (0.17 mL, 0.99 mmol) at room temperature. The reaction mixture was stirred and heated at 100° C. for 3 h. The reaction mixture was then concentrated under reduced pressure to obtained a crude product which was purified by prep-HPLC to afford 2-((6-((5-chloro-2-(4-(2-(1-(3-(2,6-dioxopiperidin-3-yl)-1-methyl-1H-indazol-6-yl)piperidin-4-yl) propan-2-yl)piperazin-1-yl)pyrimidin-4-yl)amino)-1-isopropyl-2-oxo-1,2-dihydro-1,8-naphthyridin-3-yl)oxy)-N-methylacetamide (0.016 g) as an off white solid.

¹H NMR (400 MHz, DMSO-d₆): δ 10.80 (s, 1H), 9.04 (s, 1H), 8.67 (d, J=2.4 Hz, 1H), 8.32 (d, J=2.4 Hz, 1H), 8.08 (s, 1H), 7.94-7.92 (m, 1H), 7.46 (d, J=9.2 Hz, 1H), 7.12 (s, 1H), 6.89 (d, J=8.8 Hz, 1H), 6.81 (s, 1H), 5.96 (s, 1H), 4.57 (s, 2H), 4.26-4.22 (m, 1H), 3.87-3.83 (m, 5H), 3.58 (bs, 3H), 2.69-2.59 (m, 7H), 2.30-2.14 (m, 3H), 1.83-1.74 (s, 4H), 1.70-1.55 (m, 6H), 1.39-1.38 (m, 3H), 1.21 (s, 2H), 0.98 (s, 6H).

LC-MS (ESI): m/z=853.58 [M+H]⁺

### Example 51: 2-((6-((5-chloro-2-((6R,7S)-7-((3-(2,6-dioxopiperidin-3-yl)-1-methyl-1H-indazol-7-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6R,7S)-7-((3-(2,6-dioxopiperidin-3-yl)-1-methyl-1H-indazol-7-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide] (Compound 159a)

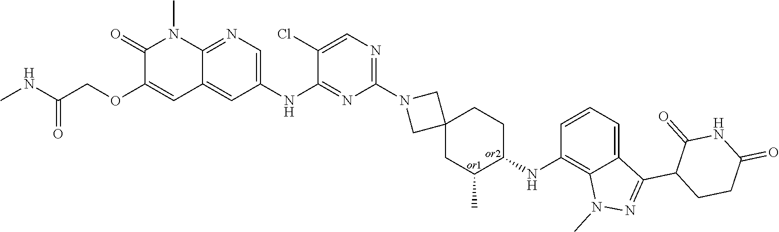 [SMILES: CNC(=O)COc1cc2cc(Nc3nc(N4CC5(CC[C@H](Nc6cccc7c(C8CCC(=O)NC8=O)nn(C)c67)[C@H](C)C5)C4)ncc3Cl)cnc2n(C)c1=O]

### Preparation of tert-butyl (6R,7S)-7-((3-(2,6-bis(benzyloxy)pyridin-3-yl)-1-methyl-1H-indazol-7-yl)amino)-6-methyl-2-azaspiro[3.5]nonane-2-carboxylate

A stirred solution of 3-(2,6-bis(benzyloxy)pyridin-3-yl)-7-bromo-1-methyl-1H-indazole (10.0 g, 19.96 mmol), tert-butyl 7-amino-6-methyl-2-azaspiro[3.5]nonane-2-carboxylate (7.61 g, 29.94 mmol) in 1,4-dioxane (100 mL) was added cesium carbonate (19.5 g, 30.0 mmol). The reaction mixture was degassed with argon for 10 minutes. Afterwards, Pd-PEPPSI-iHeptCl (0.97 g, 0.99 mmol) was added, and the reaction mixture was stirred at 100° C. for 16 h. The reaction mixture was cooled to room temperature and filtered through a celite bed. The filtrate was concentrated to get a residue which was purified by flash column chromatography (SiO₂, 230-400, 15% ethyl acetate in petroleum ether) tert-butyl (6R,7S)-7-((3-(2,6-bis(benzyloxy)pyridin-3-yl)-1-methyl-1H-indazol-7-yl)amino)-6-methyl-2-azaspiro[3.5]nonane-2-carboxylate (5.7 g) as an off white solid.

LC-MS (ESI): m/z=674.73 [M+H]⁺

### Preparation of tert-butyl (6R,7S)-7-((3-(2,6-dioxopiperidin-3-yl)-1-methyl-1H-indazol-7-yl)amino)-6-methyl-2-azaspiro[3.5]nonane-2-carboxylate

To a solution of tert-butyl (6R,7S)-7-((3-(2,6-bis(benzyloxy)pyridin-3-yl)-1-methyl-1H-indazol-7-yl)amino)-6-methyl-2-azaspiro[3.5]nonane-2-carboxylate (0.9 g, 1.33 mmol) in THF (36.0 mL) was added 20% palladium hydroxide (0.90 g) under nitrogen. The reaction mixture was stirred under H₂ (80 psi) at room temperature for 8 h. The reaction mixture was filter through a celite bed, concentrated under reduced pressure, washed with n-pentane, and dried to afford tert-butyl (6R,7S)-7-((3-(2,6-dioxopiperidin-3-yl)-1-methyl-1H-indazol-7-yl)amino)-6-methyl-2-azaspiro[3.5]nonane-2-carboxylate (0.65 g) as a green solid.

LC-MS (ESI): m/z=496.96 [M+H]⁺.

### Preparation of 3-(1-methyl-7-(((6R,7S)-6-methyl-2-azaspiro[3.5]nonan-7-yl)amino)-1H-indazol-3-yl)piperidine-2,6-dione

To a solution of tert-butyl (6R,7S)-7-((3-(2,6-dioxopiperidin-3-yl)-1-methyl-1H-indazol-7-yl)amino)-6-methyl-2-azaspiro[3.5]nonane-2-carboxylate (0.65 g, 1.31 mmol) in DCM (13.0 mL) was added TFA (6.5 mL) at 0° C. The reaction mixture was stirred at room temperature for 2 h. The reaction mixture was concentrated under reduced pressure and washed with n-pentane to afford 3-(1-methyl-7-(((6R,7S)-6-methyl-2-azaspiro[3.5]nonan-7-yl)amino)-1H-indazol-3-yl)piperidine-2,6-dione (0.6 g) as a brown solid.

LC-MS (ESI): m/z=396.46 [M+H]⁺.

### Preparation of 2-((6-((5-chloro-2-((6R,7S)-7-((3-(2,6-dioxopiperidin-3-yl)-1-methyl-1H-indazol-7-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6R,7S)-7-((3-(2,6-dioxopiperidin-3-yl)-1-methyl-1H-indazol-7-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide]

To a solution of 3-(1-methyl-7-(((6R,7S)-6-methyl-2-azaspiro[3.5]nonan-7-yl)amino)-1H-indazol-3-yl)piperidine-2,6-dione (0.3 g, 0.76 mmol) and 2-((6-((2,5-dichloropyrimidin-4-yl)amino)-1-methyl-2-oxo-1,2-dihydro-1,8-naphthyridin-3-yl)oxy)-N-methylacetamide (0.18 g, 0.45 mmol) in DMSO (6.0 mL) was added N, N-diisopropylethylamine (0.78 g, 6.08 mmol). The reaction mixture was stirred at 100° C. for 5 h. The reaction mixture was poured into ice cold water and stirred for 15 minutes. The resulting precipitate was filtered, dried under reduced pressure, and purified by prep-HPLC to afford 2-((6-((5-chloro-2-((6R,7S)-7-((3-(2,6-dioxopiperidin-3-yl)-1-methyl-1H-indazol-7-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6R,7S)-7-((3-(2,6-dioxopiperidin-3-yl)-1-methyl-1H-indazol-7-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide] (0.015 g) as an-off white solid.

¹H NMR (400 MHz, DMSO-d₆): δ 10.85 (s, 1H), 9.04 (s, 1H), 8.84 (d, J=2.00 Hz, 1H), 8.53 (s, 1H), 8.07 (s, 1H), 7.97 (d, J=4.00 Hz, 1H), 7.16 (br s, 1H), 6.87 (dd, J=8.00, 14.20 Hz, 2H), 6.64 (br s, 1H), 6.48 (d, J=6.40 Hz, 1H), 4.85 (br s, 1H), 4.64 (s, 2H), 4.26 (s, 3H), 3.74-3.67 (m, 6H), 2.97 (d, J=10.40 Hz, 1H), 2.65-2.61 (m, 6H), 2.29-2.27 (m, 1H), 2.13-2.11 (m, 4H), 1.63 (d, J=13.20 Hz, 1H), 1.40 (t, J=12.40 Hz, 2H), 1.03 (d, J=6.40 Hz, 3H), 1.77 (br s, 1H).

LC-MS (ESI): m/z=768.51 [M+H]⁺.

### Example 52: 2-((6-((5-chloro-2-((6S,7R)-7-((3-(2,6-dioxopiperidin-3-yl)-1-methyl-1H-indazol-7-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6S,7R)-7-((3-(2,6-dioxopiperidin-3-yl)-1-methyl-1H-indazol-7-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide] (Compound 159b)

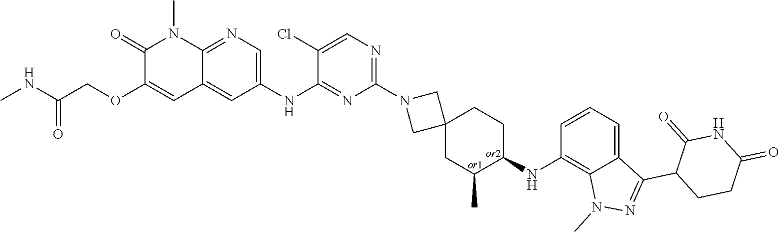 [SMILES: CNC(=O)COc1cc2cc(Nc3nc(N4CC5(CC[C@@H](Nc6cccc7c(C8CCC(=O)NC8=O)nn(C)c67)[C@@H](C)C5)C4)ncc3Cl)cnc2n(C)c1=O]

### Preparation of tert-butyl (6S,7R)-7-((3-(2,6-dioxopiperidin-3-yl)-1-methyl-1H-indazol-7-yl)amino)-6-methyl-2-azaspiro[3.5]nonane-2-carboxylate

To a solution of tert-butyl 7-((3-(2,6-bis(benzyloxy)pyridin-3-yl)-1-methyl-1H-indazol-7-yl)amino)-6-methyl-2-azaspiro[3.5]nonane-2-carboxylate (0.60 g, 0.89 mmol) in THF (30.0 mL) was added 20% Pd(OH)₂ (0.60 g). The reaction mixture was stirred under H₂ (60 psi) atmosphere at room temperature for 8 h. The reaction mixture was filtered through a celite bed and washed with THF:DCM (1:1) (500 mL). The collected filtrate was concentrated under reduced pressure to afford tert-butyl (6S,7R)-7-((3-(2,6-dioxopiperidin-3-yl)-1-methyl-1H-indazol-7-yl)amino)-6-methyl-2-azaspiro[3.5]nonane-2-carboxylate (0.45 g) as brown solid which was used in the next step without purification.

LC-MS (ESI): m/z=496.5 [M+H]⁺.

### Preparation of 3-(1-methyl-7-(((6S,7R)-6-methyl-2-azaspiro[3.5]nonan-7-yl)amino)-1H-indazol-3-yl)piperidine-2,6-dione

To a solution of tert-butyl (6S,7R)-7-((3-(2,6-dioxopiperidin-3-yl)-1-methyl-1H-indazol-7-yl)amino)-6-methyl-2-azaspiro[3.5]nonane-2-carboxylate (0.45 g, 0.90 mmol) in DCM (9.0 mL) was added TFA (0.45 mL) at 0° C. The reaction mixture was stirred at room temperature for 3 h. The reaction mixture was concentrated, co-distilled with DCM (3×40 mL), dried under reduced pressure, and triturated with diethyl ether (30 mL) to afford 3-(1-methyl-7-(((6S,7R)-6-methyl-2-azaspiro[3.5]nonan-7-yl)amino)-1H-indazol-3-yl)piperidine-2,6-dione (0.6 g) as a brown solid.

LC-MS (ESI): m/z=396.46 [M+H]⁺.

### Preparation of 2-((6-((5-chloro-2-((6S,7R)-7-((3-(2,6-dioxopiperidin-3-yl)-1-methyl-1H-indazol-7-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6S,7R)-7-((3-(2,6-dioxopiperidin-3-yl)-1-methyl-1H-indazol-7-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide]

To a solution of 3-(1-methyl-7-(((6S,7R)-6-methyl-2-azaspiro[3.5]nonan-7-yl)amino)-1H-indazol-3-yl)piperidine-2,6-dione (0.30 g, 0.76 mmol) and 2-((6-((2,5-dichloropyrimidin-4-yl)amino)-1-methyl-2-oxo-1,2-dihydro-1,8-naphthyridin-3-yl)oxy)-N-methylacetamide (0.12 g, 0.30 mmol) in DMSO (3.0 mL) was added N,N-diisopropylethylamine (0.60 mL, 3.80 mmol). The reaction mixture was stirred at 100° C. for 2 h. The reaction mixture was concentrated under reduced pressure and purified by prep-HPLC to afford 2-((6-((5-chloro-2-((6S,7R)-7-((3-(2,6-dioxopiperidin-3-yl)-1-methyl-1H-indazol-7-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6S,7R)-7-((3-(2,6-dioxopiperidin-3-yl)-1-methyl-1H-indazol-7-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide] (0.008 g) as an off white solid.

¹H NMR (400 MHz, DMSO-d₆): δ 10.84 (s, 1H), 9.03 (s, 1H), 8.84 (d, J=2.40 Hz, 1H), 8.51-8.52 (m, 1H), 8.07 (s, 1H), 7.97 (d, J=4.80 Hz, 1H), 7.16 (s, 1H), 6.86-6.84 (m, 2H), 6.48 (d, J=6.40 Hz, 1H), 4.85 (s, 1H), 4.64 (s, 2H), 4.25-4.23 (m, 4H), 3.77-3.74 (m, 5H), 3.67 (s, 2H), 2.96-2.83 (m, 1H), 2.51-2.50 (m, 4H), 2.50-2.29 (m, 1H), 2.09-2.07 (m, 4H), 1.65-1.44 (m, 2H), 1.41-1.37 (m, 2H), 1.12 (t, J=7.20 Hz, 1H), 1.03 (d, J=6.40 Hz, 3H).

LC-MS (ESI): m/z=768.5 [M+H]⁺.

### Example 53: 2-((6-((5-chloro-2-((6R,7S)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6R,7S)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide] (Compound 156b)

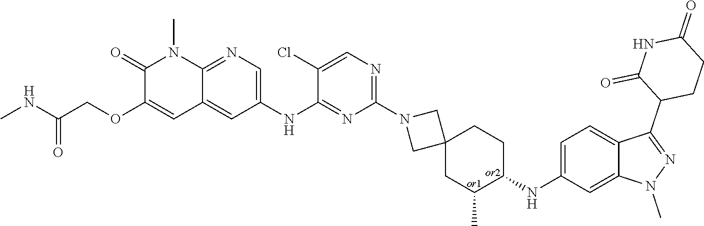 [SMILES: CNC(=O)COc1cc2cc(Nc3nc(N4CC5(CC[C@H](Nc6ccc7c(C8CCC(=O)NC8=O)nn(C)c7c6)[C@H](C)C5)C4)ncc3Cl)cnc2n(C)c1=O]

### Preparation of tert-butyl (6R,7S)-7-((3-(2,6-dioxopiperidin-3-yl)-1-methyl-1H-indazol-6-yl)amino)-6-methyl-2-azaspiro[3.5]nonane-2-carboxylate

To a solution of tert-butyl (6R,7S)-7-((3-(2,6-bis(benzyloxy)pyridin-3-yl)-1-methyl-1H-indazol-6-yl)amino)-6-methyl-2-azaspiro[3.5]nonane-2-carboxylate (0.52 g, 0.77 mmol) in THF (150 mL) was added 20% Pd(OH)₂/C (100% w/w) (0.52 g). The reaction mixture was stirred under hydrogen atmosphere (80 psi) at room temperature for 18 h. The reaction mixture was diluted with 30% THF:DCM (900 mL) and filtered through a pad of celite. The collected filtrate was concentrated under vacuum and purified by trituration with n-pentane (30 mL) to afford tert-butyl (6R,7S)-7-((3-(2,6-dioxopiperidin-3-yl)-1-methyl-1H-indazol-6-yl)amino)-6-methyl-2-azaspiro[3.5]nonane-2-carboxylate (0.44 g) as a pale brown semi-solid.

LC-MS (ESI): m/z=496.55 [M+H]⁺.

### Preparation of 3-(1-methyl-6-(((6R,7S)-6-methyl-2-azaspiro[3.5]nonan-7-yl)amino)-1H-indazol-3-yl)piperidine-2,6-dione

To a solution of tert-butyl (6R,7S)-7-((3-(2,6-dioxopiperidin-3-yl)-1-methyl-1H-indazol-6-yl)amino)-6-methyl-2-azaspiro[3.5]nonane-2-carboxylate (0.20 g, 0.40 mmol) in DCM 4.0 mL), was added TFA (2.0 mL) at 0° C. The reaction mixture was stirred at room temperature for 2 h. The reaction mixture was concentrated under vacuum and purified by trituration with DEE (30 mL) to afford 3-(1-methyl-6-(((6R,7S)-6-methyl-2-azaspiro[3.5]nonan-7-yl)amino)-1H-indazol-3-yl)piperidine-2,6-dione (0.24 g) as a pale brown semi-solid.

LC-MS (ESI): m/z=396.55 [M+H]⁺.

### Preparation of 2-((6-((5-chloro-2-((6R,7S)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6R,7S)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide]

To a stirred solution of 3-(1-methyl-6-(((6R,7S)-6-methyl-2-azaspiro[3.5]nonan-7-yl)amino)-1H-indazol-3-yl)piperidine-2,6-dione (0.25 g, 0.63 mmol) and 2-((6-((2,5-dichloropyrimidin-4-yl)amino)-1-methyl-2-oxo-1,2-dihydro-1,8-naphthyridin-3-yl)oxy)-N-methylacetamide (0.082 g, 0.20 mmol) in DMSO (5.0 mL) was added DIPEA (2.5 mL). The reaction was then stirred and heated at 100° C. for 18 h. The reaction mixture was concentrated under reduced pressure and purified by prep HPLC to afford 2-((6-((5-chloro-2-((6R,7S)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6R,7S)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide] (0.005 g) as an off-white solid.

¹H NMR (400 MHz, DMSO-d₆): § 10.81 (s, 1H), 9.04 (s, 1H), 8.83 (d, J=2.40 Hz, 1H), 8.42 (d, J=1.60 Hz, 1H), 8.06 (s, 1H), 7.95 (d, J=4.40 Hz, 1H), 7.31 (d, J=8.80 Hz, 1H), 7.25 (s, 1H), 6.69 (d, J=8.80 Hz, 1H), 6.40 (s, 1H), 5.57 (d, J=8.40 Hz, 1H), 4.62 (s, 2H), 4.16-4.15 (m, 1H), 3.79 (s, 3H), 3.67 (s, 3H), 3.61-3.58 (m, 4H), 2.68 (d, J=4.80 Hz, 3H), 2.59-2.58 (m, 3H), 2.24-2.23 (m, 1H), 2.13-2.11 (m, 1H), 1.67-1.64 (m, 6H), 1.50-1.47 (m, 1H), 0.92-0.90 (m, 3H).

LC-MS (ESI): m/z=768.47 [M+H]⁺

### Example 54: 2-((6-((5-chloro-2-((6S,7R)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6S,7R)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide] (Compound 156c)

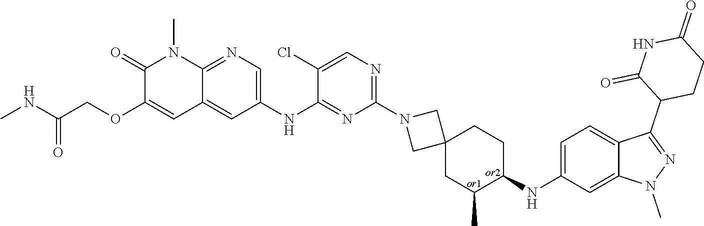 [SMILES: CNC(=O)COc1cc2cc(Nc3nc(N4CC5(CC[C@@H](Nc6ccc7c(C8CCC(=O)NC8=O)nn(C)c7c6)[C@@H](C)C5)C4)ncc3Cl)cnc2n(C)c1=O]

### Preparation of tert-butyl 6-methyl-7-oxo-2-azaspiro[3.5]nonane-2-carboxylate

To a suspension of tert-butyl 7-oxo-2-azaspiro[3.5]nonane-2-carboxylate (90.0 g, 0.38 mol) in THF (50 mL) was added LDA (0.9 L) at 0° C. The reaction mixture was stirred for 1 h. Iodomethane (70.0 mL, 1.13 mol) was then added slowly over 30 min at 0° C., and the resulting mixture was stirred at room temperature for 6 h. The reaction mixture was diluted with ice water (2 L) and extracted with ethyl acetate (2×1 L). The separated organic layer was washed with brine, dried over anhydrous Na₂SO₄, filtered, and dried under vacuum to provide a residue which was purified by column chromatography (SiO₂, 100-200 mesh, 15% ethyl acetate in petroleum ether) to afford tert-butyl 6-methyl-7-oxo-2-azaspiro[3.5]nonane-2-carboxylate (35 g) as a yellow solid.

¹H NMR (400 MHz, DMSO-d₆): δ 3.89-3.87 (m, 2H), 3.65 (s, 2H), 2.34-2.33 (m, 2H), 2.22-2.21 (m, 2H), 1.86-1.85 (m, 1H), 1.60-1.58 (m, 2H), 1.46 (s, 9H), 1.03 (d, J=6.80 Hz, 3H).

### Preparation of tert-butyl 7-amino-6-methyl-2-azaspiro[3.5]nonane-2-carboxylate

To a suspension of tert-butyl 6-methyl-7-oxo-2-azaspiro[3.5]nonane-2-carboxylate (35.0 g, 138.3 mol) in methanol (350 mL) was added NH₃ in methanol (0.35 L, 7.0 M) at room temperature. The reaction mixture was stirred for 8 h. The reaction mixture was concentrated under reduced pressure, diluted with ice water (1 L), and extracted with ethyl acetate (2×0.5 L). The separated organic layer was washed with brine and dried over anhydrous Na₂SO₄, filtered, dried under vacuum, and triturated using diethyl ether to afford tert-butyl 7-amino-6-methyl-2-azaspiro[3.5]nonane-2-carboxylate (35.0 g) as a yellow semi-solid.

LC-MS (ESI): m/z=255.28 [M+H]⁺

### Preparation of tert-butyl (6S,7R)-7-((3-(2,6-bis(benzyloxy)pyridin-3-yl)-1-methyl-1H-indazol-6-yl)amino)-6-methyl-2-azaspiro[3.5]nonane-2-carboxylate

To a solution of 3-(2,6-bis(benzyloxy)pyridin-3-yl)-6-bromo-1-methyl-1H-indazole (5.0 g, 10.0 mmol), tert-butyl 7-amino-6-methyl-2-azaspiro[3.5]nonane-2-carboxylate (10.1 g, 40.0 mmol) in 1,4-dioxane (100 mL) was added cesium carbonate (9.76 g, 30.0 mmol) under argon, followed by RuPhos (0.47 g, 1.0 mmol) and RuPhos-PdG3 (0.04 g, 0.05 mmol). The reaction mixture was stirred at 80° C. for 16 h under argon. The reaction mixture was cooled to room temperature and filtered through a celite bed. The filtrate was concentrated and purified by flash column chromatography (SiO₂, 230-400, 30% ethyl acetate in petroleum ether) to afford tert-butyl 7-((3-(2,6-bis(benzyloxy)pyridin-3-yl)-1-methyl-1H-indazol-6-yl)amino)-6-methyl-2-azaspiro[3.5]nonane-2-carboxylate (3.5 g) as an-off white solid. Diastereomeric mixture was subjected to SFC separation to isolate four isomers. The first eluting isomer was assigned arbitrarily as tert-butyl (6S,7R)-7-((3-(2,6-bis(benzyloxy)pyridin-3-yl)-1-methyl-1H-indazol-6-yl)amino)-6-methyl-2-azaspiro[3.5]nonane-2-carboxylate (0.60 g).

LC-MS (ESI): m/z=674.43 [M+H]⁺

### Preparation of tert-butyl (6S,7R)-7-((3-(2,6-dioxopiperidin-3-yl)-1-methyl-1H-indazol-6-yl)amino)-6-methyl-2-azaspiro[3.5]nonane-2-carboxylate

A stirred solution of tert-butyl (6S,7R)-7-((3-(2,6-bis(benzyloxy)pyridin-3-yl)-1-methyl-1H-indazol-6-yl)amino)-6-methyl-2-azaspiro[3.5]nonane-2-carboxylate (0.60 g, 0.90 mmol) in THF (25 mL) was degassed with nitrogen. 20% Palladium hydroxide (0.60 g) was then added. The resulting mixture was stirred under H₂ (80 psi) at room temperature for 16 h. The reaction mixture was filtered through a celite bed, concentrated, washed with n-pentane, and dried to afford tert-butyl (6S,7R)-7-((3-(2,6-dioxopiperidin-3-yl)-1-methyl-1H-indazol-6-yl)amino)-6-methyl-2-azaspiro[3.5]nonane-2-carboxylate (0.45 g) as a green solid.

LC-MS (ESI): m/z=496.40 [M+H]⁺

### Preparation of 3-(1-methyl-6-(((6S,7R)-6-methyl-2-azaspiro[3.5]nonan-7-yl)amino)-1H-indazol-3-yl)piperidine-2,6-dione

To a stirred solution of tert-butyl (6S,7R)-7-((3-(2,6-dioxopiperidin-3-yl)-1-methyl-1H-indazol-6-yl)amino)-6-methyl-2-azaspiro[3.5]nonane-2-carboxylate (0.45 g, 0.90 mmol)) in DCM (4.5 mL), was added TFA (4.5 mL) at 0° C. The reaction mixture was stirred at room temperature for 2 h. The reaction mixture was concentrated under reduced pressure and washed with n-pentane to afford 3-(1-methyl-6-(((6S,7R)-6-methyl-2-azaspiro[3.5]nonan-7-yl)amino)-1H-indazol-3-yl)piperidine-2,6-dione (0.3 g) as a brown solid.

LC-MS (ESI): m/z=395.63 [M+H]⁺.

### Preparation of 2-((6-((5-chloro-2-((6S,7R)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6S,7R)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide]

To a solution of 3-(1-methyl-6-(((6S,7R)-6-methyl-2-azaspiro[3.5]nonan-7-yl)amino)-1H-indazol-3-yl)piperidine-2,6-dione (0.15 g, 0.38 mmol) and 2-((6-((2,5-dichloropyrimidin-4-yl)amino)-1-methyl-2-oxo-1,2-dihydro-1,8-naphthyridin-3-yl)oxy)-N-methylacetamide (0.09 g, 0.23 mmol) in DMSO (3 mL) was added N,N-diisopropylethylamine (0.39 g, 3.04 mmol). The reaction mixture was stirred at 100° C. for 5 h. The reaction mixture was poured into ice cold water and stirred for 15 minutes. The resulting precipitate was filtered, dried under reduced pressure, and purified by prep-HPLC to afford 2-((6-((5-chloro-2-((6S,7R)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6S,7R)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide] (0.006 g) as an off white solid.

¹H NMR (400 MHz, DMSO-d₆): δ. 10.81 (s, 1H), 9.04 (s, 1H), 8.84 (s, 1H), 8.48 (s, 1H), 8.07 (s, 1H), 7.97 (d, J=4.40 Hz, 1H), 7.29 (d, J=8.80 Hz, 1H), 7.20 (s, 1H), 6.50 (d, J=8.80 Hz, 1H), 6.36 (s, 1H), 5.65 (d, J=9.20 Hz, 1H), 4.65 (s, 1H), 4.14-4.16 (m, 1H), 3.74-3.67 (m, 10H), 3.00-2.98 (m, 1H), 2.68-2.67 (m, 3H), 2.60-2.58 (m, 2H), 2.26-2.24 (m, 1H), 2.16-2.15 (m, 1H), 2.07-1.99 (m, 3H), 1.65 (t, J=12.80 Hz, 1H), 1.50 (s, 1H), 1.39-1.36 (m, 1H), 1.24 (s, 1H), 1.16 (t, J=7.20 Hz, 1H), 0.97 (d, J=6.00 Hz, 3H).

LC-MS (ESI): m/z=768.47 [M+H]⁺.

### Example 55: 2-((6-((5-chloro-2-((6R,7R)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6R,7R)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide] (Compound 156d)

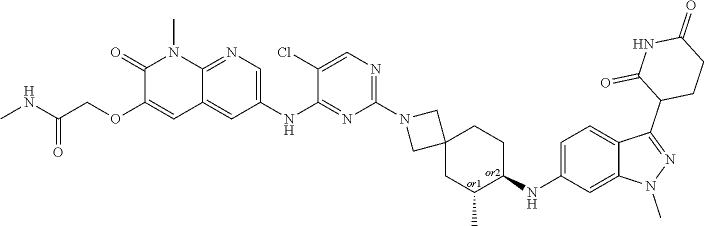 [SMILES: CNC(=O)COc1cc2cc(Nc3nc(N4CC5(CC[C@@H](Nc6ccc7c(C8CCC(=O)NC8=O)nn(C)c7c6)[C@H](C)C5)C4)ncc3Cl)cnc2n(C)c1=O]

### Preparation of tert-butyl (6R,7R)-7-((3-(2,6-dioxopiperidin-3-yl)-1-methyl-1H-indazol-6-yl)amino)-6-methyl-2-azaspiro[3.5]nonane-2-carboxylate

To a solution of tert-butyl (6R,7R)-7-((3-(2,6-bis(benzyloxy)pyridin-3-yl)-1-methyl-1H-indazol-6-yl)amino)-6-methyl-2-azaspiro[3.5]nonane-2-carboxylate (0.20 g, 0.29 mmol) in THF (8.0 mL) was added 20% palladium hydroxide (0.20 g) under N₂. The reaction mixture was stirred under H₂ (80 psi) pressure at room temperature for 16 h. The reaction mixture was filtered through a celite bed, concentrated under reduced pressure, washed with n-pentane, and dried to afford tert-butyl (6R,7R)-7-((3-(2,6-dioxopiperidin-3-yl)-1-methyl-1H-indazol-6-yl)amino)-6-methyl-2-azaspiro[3.5]nonane-2-carboxylate (0.14 g) as a brown solid.

LC-MS (ESI): m/z=496.60 [M+H]⁺.

### Preparation of 3-(1-methyl-6-(((6R,7R)-6-methyl-2-azaspiro[3.5]nonan-7-yl)amino)-1H-indazol-3-yl)piperidine-2,6-dione

To a solution of tert-butyl (6R,7R)-7-((3-(2,6-dioxopiperidin-3-yl)-1-methyl-1H-indazol-6-yl)amino)-6-methyl-2-azaspiro[3.5]nonane-2-carboxylate (0.14 g, 1.28 mmol)) in DCM (3.0 mL) cooled to 0° C. was added TFA (1.40 mL). The reaction mixture was stirred at room temperature for 2 h. The reaction mixture was concentrated under reduced pressure and washed with n-pentane to provide 3-(1-methyl-6-(((6R,7R)-6-methyl-2-azaspiro[3.5]nonan-7-yl)amino)-1H-indazol-3-yl)piperidine-2,6-dione (0.2 g) as a brown solid.

LC-MS (ESI): m/z=396.46 [M+H]⁺

### Preparation of 2-((6-((5-chloro-2-((6R,7R)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6R,7R)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide]

To a solution of 3-(1-methyl-6-(((6R,7R)-6-methyl-2-azaspiro[3.5]nonan-7-yl)amino)-1H-indazol-3-yl)piperidine-2,6-dione (0.10 g, 0.20 mmol) and 2-((6-((2,5-dichloropyrimidin-4-yl)amino)-1-methyl-2-oxo-1,2-dihydro-1,8-naphthyridin-3-yl)oxy)-N-methylacetamide (0.04 g, 0.12 mmol) in DMSO (2.0 mL) was added N,N-diisopropylethylamine (0.21 g, 1.62 mmol). The reaction mixture was stirred at 100° C. for 12 h. The reaction mixture was poured into ice cold water and stirred for 15 minutes. The resulting precipitate was filtered, dried, and purified by prep-HPLC to afford 2-((6-((5-chloro-2-((6R,7R)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide [rel-2-((6-((5-chloro-2-((6R,7R)-7-((3-(2,6-dioxopiperidin-3-yl)-1-methyl-1H-indazol-6-yl)amino)-6-methyl-2-azaspiro[3.5]nonan-2-yl)pyrimidin-4-yl)amino)-1-methyl-2-oxo-1,2-dihydro-1,8-naphthyridin-3-yl)oxy)-N-methylacetamide] (0.009 g) as an off white solid.

¹H NMR (400 MHz, DMSO-d₆): δ 10.81 (s, 1H), 9.03 (s, 1H), 8.83 (d, J=4.00 Hz, 1H), 8.42 (d, J=4.00 Hz, 1H), 8.05 (s, 1H), 7.95 (d, J=4.00 Hz, 1H), 7.30 (d, J=8.00 Hz, 1H), 7.24 (s, 1H), 6.68 (d, J=8.00 Hz, 1H), 6.40 (s, 1H), 5.56 (d, J=8.00 Hz, 1H), 4.62 (s, 2H), 4.18-4.14 (m, 1H), 3.79-3.66 (m, 10H), 3.61-3.60 (m, 1H), 2.68 (d, J=4.00 Hz, 3H), 2.59 (d, J=4.00 Hz, 2H), 2.27-2.22 (m, 1H), 2.17-2.11 (m, 1H), 1.84-1.71 (m, 4H), 1.67-1.60 (m, 2H), 1.49-1.47 (m, 1H), 0.91 (d, J=4.00 Hz, 3H).

LC-MS (ESI): m/z=768.47 [M+H]⁺.

### Example 56: 2-((6-((5-chloro-2-(4-((1-(3-(2,6-dioxopiperidin-3-yl)-1-methyl-1H-indazol-7-yl)piperidin-4-yl)methyl)piperazin-1-yl)pyridin-4-yl)amino)-1-methyl-2-oxo-1,2-dihydro-1,8-naphthyridin-3-yl)oxy)-N-methylacetamide (Compound 160)

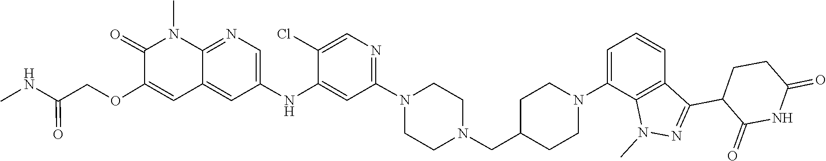 [SMILES: CNC(=O)COc1cc2cc(Nc3cc(N4CCN(CC5CCN(c6cccc7c(C8CCC(=O)NC8=O)nn(C)c67)CC5)CC4)ncc3Cl)cnc2n(C)c1=O]

### Preparation of tert-butyl 4-(5-chloro-4-iodopyridin-2-yl)piperazine-1-carboxylate

To a stirred solution of 5-chloro-2-fluoro-4-iodopyridine (1.00 g, 3.88 mmol) in DMSO (10 mL) and DIPEA (3.43 mL, 19.4 mmol) was added tert-butyl piperazine-1-carboxylate (0.72 g, 3.88 mmol). The reaction mixture was stirred at 100° C. for 16 h. The reaction mixture was cooled to room temperature, diluted with water (50 mL), and extracted with ethyl acetate (30 mL×2). The combined organic layer was dried over anhydrous Na₂SO₄, filtered, and concentrated under reduced pressure to afford a crude product which was purified by flash chromatography (230-400 mesh silica gel; 10-15% ethyl acetate in petroleum ether) to afford tert-butyl 4-(5-chloro-4-iodopyridin-2-yl)piperazine-1-carboxylate as a white solid (0.71 g).

LC-MS (ESI): m/z=424.02 [M+H]⁺

### Preparation of tert-butyl 4-(5-chloro-4-((8-methyl-6-(2-(methylamino)-2-oxoethoxy)-7-oxo-7,8-dihydro-1,8-naphthyridin-3-yl)amino)pyridin-2-yl)piperazine-1-carboxylate

To a stirred solution of tert-butyl 4-(5-chloro-4-iodopyridin-2-yl)piperazine-1-carboxylate (0.70 g, 1.65 mmol) and 2-((6-amino-1-methyl-2-oxo-1,2-dihydro-1,8-naphthyridin-3-yl)oxy)-N-methylacetamide (0.65 g, 2.47 mmol) in 1,4-dioxane (14 mL) was added cesium carbonate (1.61 g, 4.95 mmol). The reaction mixture was degassed with argon for 15 minutes. Then Ruphos (0.08 g, 0.17 mmol) and Ruphos Pd G3 (0.07 g, 0.08 mmol) were added under argon. The reaction mixture was then stirred at 100° C. for 16 h. The reaction mixture was diluted with ethyl acetate (40 mL), filtered through a celite bed, and washed with 10% MeOH:DCM (250 mL). The filtrate was concentrated and dried under vacuum to obtain a crude product which was purified by reverse phase flash chromatography (C₁₈ silica gel; 20-25% 0.1% formic acid in MeCN) to afford tert-butyl 4-(5-chloro-4-((8-methyl-6-(2-(methylamino)-2-oxoethoxy)-7-oxo-7,8-dihydro-1,8-naphthyridin-3-yl)amino)pyridin-2-yl)piperazine-1-carboxylate (0.15 g) as a white solid.

LC-MS (ESI): m/z=91.06 [M+H]⁺

### Preparation of 2-((6-((5-chloro-2-(piperazin-1-yl)pyridin-4-yl)amino)-1-methyl-2-oxo-1,2-dihydro-1,8-naphthyridin-3-yl)oxy)-N-methylacetamide

To a stirred solution of tert-butyl 4-(5-chloro-4-((8-methyl-6-(2-(methylamino)-2-oxoethoxy)-7-oxo-7,8-dihydro-1,8-naphthyridin-3-yl)amino)pyridin-2-yl)piperazine-1-carboxylate (0.15 g, 0.26 mmol) in DCM (1.5 mL) was added TFA (0.75 mL) at 0° C. The reaction mixture was stirred at room temperature for 3 h. The reaction mixture was concentrated under reduced pressure, co-distilled with DCM (3×10 mL), and dried to afford 2-((6-((5-chloro-2-(piperazin-1-yl)pyridin-4-yl)amino)-1-methyl-2-oxo-1,2-dihydro-1,8-naphthyridin-3-yl)oxy)-N-methylacetamide (0.20 g) as a brown solid which was used in the next step without purification.

LC-MS (ESI): m/z=458.19 [M+H]⁺

### Preparation of (1-(3-(2,6-bis(benzyloxy)pyridin-3-yl)-1-methyl-1H-indazol-7-yl)piperidin-4-yl)methanol

To a stirred solution of 3-(2,6-bis(benzyloxy)pyridin-3-yl)-7-bromo-1-methyl-1H-indazole (1.0 g, 1.99 mmol), and piperidin-4-ylmethanol (0.92 g, 7.99 mmol) in 1,4-dioxane (20 mL) was added cesium carbonate (1.95 g, 5.99 mmol). The reaction mixture was degassed with argon for 10 minutes. Then was added Pd-PEPPSI-iHeptCl (0.097 g, 0.10 mmol). The mixture was again degassed for 5 min. The reaction mixture was stirred at 100° C. for 16 h. The reaction mixture was cooled to room temperature, diluted with ethyl acetate, and filtered through a celite bed. The filtrate was concentrated to obtain a crude product which was purified by flash column chromatography (SiO₂, 230-400 mesh, 30-35% ethyl acetate in petroleum ether) to afford (1-(3-(2,6-bis(benzyloxy)pyridin-3-yl)-1-methyl-1H-indazol-7-yl)piperidin-4-yl)methanol (0.32 g) as a brown sticky compound.

LC-MS (ESI): 535.60 [M+H]⁺

### Preparation of 3-(7-(4-(hydroxymethyl)piperidin-1-yl)-1-methyl-1H-indazol-3-yl)piperidine-2,6-dione

To a stirred solution of (1-(3-(2,6-bis(benzyloxy)pyridin-3-yl)-1-methyl-1H-indazol-7-yl)piperidin-4-yl)methanol (0.30 g, 0.56 mmol) in THF (12 mL) was added 20% Pd(OH)₂/C (0.30 g, 100% w/w). The reaction mixture was stirred at room temperature under H₂ atmosphere (60 psi) for 5 h. The reaction mixture was diluted with THF (50 mL), filtered through a celite bed, and washed with 200 mL of THF:DCM (1:1). The filtrate was concentrated and dried to afford 3-(7-(4-(hydroxymethyl)piperidin-1-yl)-1-methyl-1H-indazol-3-yl)piperidine-2,6-dione (0.21 g) as a brown solid which was used in the next step without purification.

LC-MS (ESI): m/z=357.19 [M+H]

### Preparation of 1-(3-(2,6-dioxopiperidin-3-yl)-1-methyl-1H-indazol-7-yl)piperidine-4-carbaldehyde

To a stirred solution of 3-(7-(4-(hydroxymethyl)piperidin-1-yl)-1-methyl-1H-indazol-3-yl)piperidine-2,6-dione (0.20 g, 0.56 mmol) in DCM (20 mL) was added Dess-Martin periodinane (0.71 g, 1.68 mmol) at 0° C. The reaction mixture was stirred at room temperature for 4 h. The reaction mixture was extracted with DCM (50 mL×2), and the combined organic layer was dried over anhydrous Na₂SO₄, filtered, and concentrated under reduced pressure to afford 1-(3-(2,6-dioxopiperidin-3-yl)-1-methyl-1H-indazol-7-yl)piperidine-4-carbaldehyde as a brown gummy compound (0.25 g) which was used in the next step without further purification.

LC-MS (ESI): m/z=355.46 [M+H]⁺

### Preparation of 2-((6-((5-chloro-2-(4-((1-(3-(2,6-dioxopiperidin-3-yl)-1-methyl-1H-indazol-7-yl)piperidin-4-yl)methyl)piperazin-1-yl)pyridin-4-yl)amino)-1-methyl-2-oxo-1,2-dihydro-1,8-naphthyridin-3-yl)oxy)-N-methylacetamide

To a stirred solution of 1-(3-(2,6-dioxopiperidin-3-yl)-1-methyl-1H-indazol-7-yl)piperidine-4-carbaldehyde (0.1 g, 0.28 mmol) and 2-((6-((5-chloro-2-(piperazin-1-yl)pyridin-4-yl)amino)-1-methyl-2-oxo-1,2-dihydro-1,8-naphthyridin-3-yl)oxy)-N-methylacetamide (0.077 g, 0.16 mmol) in DCM (2 mL) was added TEA (0.50 mL). The reaction mixture was refluxed for 1 h. The reaction mixture was then cooled to room temperature, treated with sodium triacetoxyborohydride (0.17 g, 0.84 mmol) at 0° C., and stirred at room temperature for 5 h. The reaction mixture was concentrated under reduced pressure to provide a crude product which was purified by prep-HPLC to afford 2-((6-((5-chloro-2-(4-((1-(3-(2,6-dioxopiperidin-3-yl)-1-methyl-1H-indazol-7-yl)piperidin-4-yl)methyl)piperazin-1-yl)pyridin-4-yl)amino)-1-methyl-2-oxo-1,2-dihydro-1,8-naphthyridin-3-yl)oxy)-N-methylacetamide (0.024 g) as an off white solid.

¹H NMR, 400 MHz, DMSO-d6: δ 10.88 (s, 1H), 8.46 (d, J=2.80 Hz, 2H), 8.02-7.93 (m, 3H), 7.36 (dd, J=2.40, 6.60 Hz, 1H), 7.30 (s, 1H), 7.02-6.99 (m, 2H), 6.15 (s, 1H), 4.59 (s, 2H), 4.32 (q, J=5.20 Hz, 1H), 4.23 (s, 3H), 3.74 (s, 3H), 3.26 (s, 6H), 2.67-2.60 (m, 7H), 2.39 (br s, 4H), 2.30-2.12 (m, 4H), 1.89-1.84 (m, 2H), 1.68 (br s, 1H), 1.38-1.36 (m, 2H).

LC-MS (ESI): m/z=796.49 [M+H]⁺

### Example 57: 2-((6-((5-chloro-2-(4-((1-(3-(2,4-dioxotetrahydropyrimidin-1 (2H)-yl)-1-methyl-1H-indazol-7-yl)piperidin-4-yl)methyl)piperazin-1-yl)pyrimidin-4-yl)amino)-1-methyl-2-oxo-1,2-dihydro-1,8-naphthyridin-3-yl)oxy)-N-methylacetamide (Compound

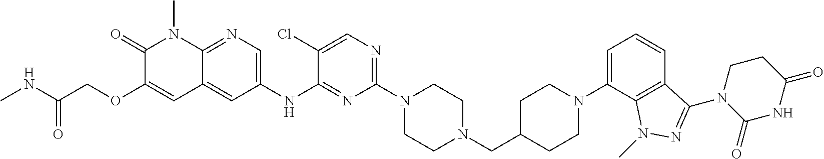 [SMILES: CNC(=O)COc1cc2cc(Nc3nc(N4CCN(CC5CCN(c6cccc7c(N8CCC(=O)NC8=O)nn(C)c67)CC5)CC4)ncc3Cl)cnc2n(C)c1=O]

### Preparation of 7-bromo-1-methyl-1H-indazol-3-amine

A solution of 3-bromo-2-fluorobenzonitrile (25.00 g, 124.99 mmol) in ethanol (400 mL) was degassed with nitrogen for 10 minutes, followed by the addition of methyl hydrazine (85% in aqueous) (11.52 g, 249.99 mmol) at 0° C. The resulting mixture was then stirred at 120° C. for 18 h and concentrated under vacuum to obtain a residue, which was then purified by column chromatography (SiO₂; 40% EtOAc in petroleum ether) to afford 7-bromo-1-methyl-1H-indazol-3-amine (18.0 g) as a brown semi-solid.

LC-MS (ESI): m/z=226.09 [M+H]⁺

### Preparation of 3-((7-bromo-1-methyl-1H-indazol-3-yl)amino) propanoic acid

To a stirred solution of 7-bromo-1-methyl-1H-indazol-3-amine (17.00 g, 75.20 mmol) in HCl (170 mL, 2N), acrylic acid (8.90 mL, 99.52 mmol) and TBAB (4.84 g, 15.04 mmol) were added. The reaction mixture was heated to 100° C. for 18 h. The reaction mixture was then quenched with ice cold water (200 mL) and extracted with 10% MeOH in ethyl acetate (5×100 mL). The separated organic layers were combined, washed with brine (100 mL), dried over anhydrous Na₂SO₄, filtered, and concentrated under vacuum to afford 3-((7-bromo-1-methyl-1H-indazol-3-yl)amino) propanoic acid (15.6 g) as a pale brown semi-solid which was used in the next step without further purification.

LC-MS (ESI): m/z=298.03 [M+H]⁺.

### Preparation of 1-(7-bromo-1-methyl-1H-indazol-3-yl) dihydropyrimidine-2,4 (1H,3H)-dione

To a stirred solution of 3-((7-bromo-1-methyl-1H-indazol-3-yl)amino) propanoic acid (15.00 g, 50.31 mmol) in acetic acid (150 mL) was added sodium cyanate (13.08 g, 201.25 mmol). The reaction mixture was heated to 75° C. for 18 h, followed by the addition of 4N HCl (180 mL). The resulting mixture was then stirred for an additional 5 h. The reaction mixture was quenched with ice cold water (500 mL). The precipitated solid was filtered, dried under vacuum, and washed with n-pentane to afford 1-(7-bromo-1-methyl-1H-indazol-3-yl) dihydropyrimidine-2,4 (1H,3H)-dione (3.91 g) as a brown solid.

LC-MS (ESI): m/z=324.33 [M+H]⁺.

### Preparation of tert-butyl 4-((1-(3-(2,4-dioxotetrahydropyrimidin-1 (2H)-yl)-1-methyl-1H-indazol-7-yl)piperidin-4-yl)methyl)piperazine-1-carboxylate

A stirred solution of 1-(7-bromo-1-methyl-1H-indazol-3-yl) dihydropyrimidine-2,4 (1H,3H)-dione (3.10 g, 9.59 mmol), tert-butyl 4-(piperidin-4-ylmethyl)piperazine-1-carboxylate (9.52 g, 33.58 mmol) and Cs₂CO₃ (15.63 g, 47.96 mmol) in dioxane (31 mL) and DMF (15.50 mL) was degassed with argon for 10 minutes. Pd-PEPPSI-iHeptCl (0.47 g, 0.48 mmol) was then added. The resulting mixture was heated to 120° C. for 2 h under microwave irradiation. The reaction mixture was filtered through a celite bed, concentrated under vacuum, and purified by column chromatography (SiO₂, 100-200 mesh; 80-90% EtOAc in petroleum ether) to afford tert-butyl 4-((1-(3-(2,4-dioxotetrahydropyrimidin-1 (2H)-yl)-1-methyl-1H-indazol-7-yl) piperidin-4-yl)methyl)piperazine-1-carboxylate (1.22 g) as a pale brown semi-solid.

LC-MS (ESI): m/z=526.67 (M+H)⁺.

### Preparation of 1-(1-methyl-7-(4-(piperazin-1-ylmethyl)piperidin-1-yl)-1H-indazol-3-yl) dihydropyrimidine-2,4 (1H,3H)-dione

To a solution of tert-butyl 4-((1-(3-(2,4-dioxotetrahydropyrimidin-1 (2H)-yl)-1-methyl-1H-indazol-7-yl)piperidin-4-yl)methyl)piperazine-1-carboxylate (1.20 g, 2.28 mmol) in DCM (12.0 mL) was added TFA (6.0 mL) at 0° C. The reaction mixture was stirred at room temperature for 1 h. The reaction mixture was concentrated under reduced pressure and triturated with n-pentane to afford 1-(1-methyl-7-(4-(piperazin-1-ylmethyl)piperidin-1-yl)-1H-indazol-3-yl) dihydropyrimidine-2,4 (1H,3H)-dione (0.95 g) as a pale brown semi solid.

LC-MS (ESI): m/z=426.49 [M+H]⁺.

### Preparation of 2-((6-((5-chloro-2-(4-((1-(3-(2,4-dioxotetrahydropyrimidin-1 (2H)-yl)-1-methyl-1H-indazol-7-yl)piperidin-4-yl)methyl)piperazin-1-yl)pyrimidin-4-yl)amino)-1-methyl-2-oxo-1,2-dihydro-1,8-naphthyridin-3-yl)oxy)-N-methylacetamide

To a stirred solution of 1-(1-methyl-7-(4-(piperazin-1-ylmethyl)piperidin-1-yl)-1H-indazol-3-yl) dihydropyrimidine-2,4 (1H,3H)-dione (0.50 g, 1.17 mmol) and 2-((6-((2,5-dichloropyrimidin-4-yl)amino)-1-methyl-2-oxo-1,2-dihydro-1,8-naphthyridin-3-yl)oxy)-N-methylacetamide (0.19 g, 0.47 mmol) in DMSO (3.0 mL) was added DIPEA (3.0 mL). The reaction mixture was stirred and heated at 100° C. for 18 hours. The reaction mixture was concentrated under reduced pressure to obtain crude product. The crude product was purified by prep-HPLC to afford 2-((6-((5-chloro-2-(4-((1-(3-(2,4-dioxotetrahydropyrimidin-1 (2H)-yl)-1-methyl-1H-indazol-7-yl)piperidin-4-yl)methyl)piperazin-1-yl)pyrimidin-4-yl)amino)-1-methyl-2-oxo-1,2-dihydro-1,8-naphthyridin-3-yl)oxy)-N-methylacetamide (0.20 g, 21%) as an off white solid.

¹H NMR (400 MHz, DMSO-d₆): § 10.53 (s, 1H), 9.09 (s, 1H), 8.70 (d, J=2.40 Hz, 1H), 8.31 (d, J=2.40 Hz, 1H), 8.26 (s, 1H), 8.09 (br s, 1H), 7.94 (d, J=4.80 Hz, 1H), 7.28-7.27 (m, 1H), 7.19 (s, 1H), 7.01 (d, J=4.80 Hz, 2H), 4.63 (s, 2H), 4.23 (s, 3H), 3.88-3.86 (m, 2H), 3.74 (s, 3H), 3.62 (s, 4H), 3.32-3.25 (m, 2H), 2.77-2.75 (m, 2H), 2.74-2.72 (m, 5H), 2.40-2.32 (m, 4H), 2.26-2.24 (m, 2H), 1.91-1.87 (m, 2H), 1.40-1.37 (m, 2H).

LC-MS (ESI): m/z=798.50 [M+H]⁺

### Example 58. 2-((6-((5-chloro-2-((3R)-4-((1-(3-(2,6-dioxopiperidin-3-yl)-1-methyl-1H-indazol-7-yl)piperidin-4-yl)methyl)-3-(trifluoromethyl)piperazin-1-yl)pyrimidin-4-yl)amino)-1-methyl-2-oxo-1,2-dihydro-1,8-naphthyridin-3-yl)oxy)-N-methylacetamide (Compound 141a)

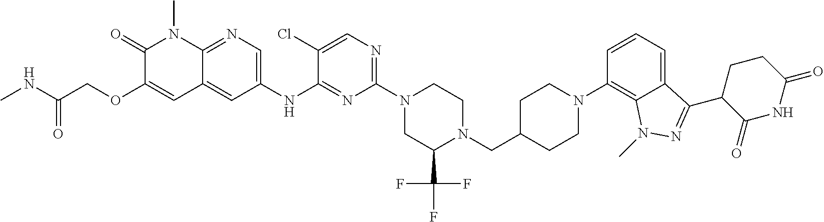 [SMILES: CNC(=O)COc1cc2cc(Nc3nc(N4CCN(CC5CCN(c6cccc7c(C8CCC(=O)NC8=O)nn(C)c67)CC5)[C@@H](C(F)(F)F)C4)ncc3Cl)cnc2n(C)c1=O]

### Preparation of tert-butyl (R)-4-((1-((benzyloxy) carbonyl)piperidin-4-yl)methyl)-3-(trifluoromethyl)piperazine-1-carboxylate

A one dram vial containing decaborane (14) (230.3 mg, 1.884 mmol) was charged with tert-butyl (R)-3-(trifluoromethyl)piperazine-1-carboxylate (1.597 g, 6.281 mmol) and benzyl 4-formylpiperidine-1-carboxylate (1.610 g, 1.319 mL, 6.511 mmol). A stirrer bar was added, followed by wet MeOH (15.00 mL). The reaction mixture was stirred at room temperature for 18 h. The reaction mixture was quenched by the addition of 1.0 M HCl (1.5 mL). The aqueous layer was extracted with DCM (3×). The combined organic layers were concentrated under reduced pressure and purified by column chromatography (0-100% (3:1 EtOAc/EtOH)/heptane) to afford tert-butyl (R)-4-((1-((benzyloxy) carbonyl)piperidin-4-yl)methyl)-3-(trifluoromethyl)piperazine-1-carboxylate (2.390 g).

LC-MS (ESI): m/z=486.7 [M+H]⁺.

### Preparation of tert-butyl (R)-4-(piperidin-4-ylmethyl)-3-(trifluoromethyl)piperazine-1-carboxylate

To a 30 mL vial containing tert-butyl (R)-4-((1-((benzyloxy) carbonyl)piperidin-4-yl)methyl)-3-(trifluoromethyl)piperazine-1-carboxylate (2.393 g, 4.928 mmol) was added isopropanol (6.00 mL) and ethyl acetate (3.00 mL). The reaction mixture was passed through a Pd(OH)₂ cartridge in an H-Cube with a flow rate of 1 mL/min at 60° C. and 20 bar for 3 h. The volatiles of the reaction mixture were evaporated under reduced pressure. The resulting mixture was further dried under vacuum to afford tert-butyl (R)-4-(piperidin-4-ylmethyl)-3-(trifluoromethyl)piperazine-1-carboxylate (1.673 g, 4.761 mmol) which was used in the next step without further purification.

LC-MS (ESI): m/z=352.5 [M+H]⁺.

### Preparation of tert-butyl (R)-4-((1-(3-(2,6-bis(benzyloxy)pyridin-3-yl)-1-methyl-1H-indazol-7-yl)piperidin-4-yl)methyl)-3-(trifluoromethyl)piperazine-1-carboxylate

Dry N_{2(g)} was bubbled through a mixture of tert-butyl (R)-4-(piperidin-4-ylmethyl)-3-(trifluoromethyl)piperazine-1-carboxylate (1.00 g, 2.85 mmol), 3-(2,6-bis(benzyloxy)pyridin-3-yl)-7-bromo-1-methyl-1H-indazole (1.69 g, 3.38 mmol), and RuPhos-Pd-G3 (185 mg, 221 μmol) in THF (15.0 mL) for 10 minutes. While still sparging the solution with N₂ (g), a solution of KOt-Bu (8.25 mL, 1.00 M in THF, 8.25 mmol) was added, and the reaction mixture was stirred and heated at 70° C. for 6 h. The reaction mixture was then treated with formic acid (644 μL, 17.1 mmol) and purified by column chromatography (0-60% (3:1 EtOAc/EtOH)/heptane) to afford tert-butyl (R)-4-((1-(3-(2,6-bis(benzyloxy)pyridin-3-yl)-1-methyl-1H-indazol-7-yl)piperidin-4-yl)methyl)-3-(trifluoromethyl)piperazine-1-carboxylate (1.480 g, 1.920 mmol).

LC-MS (ESI): m/z=671.5 [M-Boc+H]⁺.

### Preparation of tert-butyl (3R)-4-((1-(3-(2,6-dioxopiperidin-3-yl)-1-methyl-1H-indazol-7-yl)piperidin-4-yl)methyl)-3-(trifluoromethyl)piperazine-1-carboxylate

A solution of tert-butyl (R)-4-((1-(3-(2,6-bis(benzyloxy)pyridin-3-yl)-1-methyl-1H-indazol-7-yl)piperidin-4-yl)methyl)-3-(trifluoromethyl)piperazine-1-carboxylate (1.475 g, 1.913 mmol) in EtOAc (10.0 mL), iPrOH (10.0 mL), and DMF (5.00 mL) was sparged for 5 minutes with N_{2(g)}. The reaction mixture was then charged with Pd/C (1.198 g, 10% Wt, 1.126 mmol), sparged with H_{2(g)} for 10 minutes with vigorous stirring, sealed, and warmed to 50° C. under an atmosphere of H_{2(g)} for 3 days. The reaction mixture was sparged with N_{2(g)} for 5 minutes, charged with additional Pd/C (1.021 g, 10% Wt, 959.4 μmol), and then sparged with H_{2(g)} for 10 min. The reaction vessel was sealed and warmed to 50° C. under an atmosphere of H₂ (g) for 20 h. The reaction mixture was then cooled to room temperature, filtered through a pad of celite, and rinsed with EtOAc, MeOH, then DCM. The mixture was concentrated under reduced pressure. The residue was dissolved in minimal DMSO and purified by reverse-phase column chromatography (10-100% MeCN—H₂O [+0.1% formic acid]) to afford tert-butyl (3R)-4-((1-(3-(2,6-dioxopiperidin-3-yl)-1-methyl-1H-indazol-7-yl)piperidin-4-yl)methyl)-3-(trifluoromethyl)piperazine-1-carboxylate (443 mg).

LC-MS (ESI): m/z=593.4 [M+H]⁺.

### Preparation of 3-(1-methyl-7-(4-(((R)-2-(trifluoromethyl)piperazin-1-yl)methyl)piperidin-1-yl)-1H-indazol-3-yl)piperidine-2,6-dione

A solution of tert-butyl (3R)-4-((1-(3-(2,6-dioxopiperidin-3-yl)-1-methyl-1H-indazol-7-yl)piperidin-4-yl)methyl)-3-(trifluoromethyl)piperazine-1-carboxylate (442 mg, 746 μmol) in DCM (1.00 mL) was treated with a solution of TFA (1.00 mL, 13.0 mmol) and DCM (1.00 mL). The reaction mixture was stirred at an ambient temperature for 90 min. The mixture was then concentrated under reduced pressure. The residue was suspended in DCM and concentrated under reduced pressure again to afford 3-(1-methyl-7-(4-(((R)-2-(trifluoromethyl)piperazin-1-yl)methyl)piperidin-1-yl)-1H-indazol-3-yl)piperidine-2,6-dione (416 mg).

LC-MS (ESI): m/z=493.4 [M+H]⁺.

### Preparation of 2-((6-((5-chloro-2-((3R)-4-((1-(3-(2,6-dioxopiperidin-3-yl)-1-methyl-1H-indazol-7-yl)piperidin-4-yl)methyl)-3-(trifluoromethyl)piperazin-1-yl)pyrimidin-4-yl)amino)-1-methyl-2-oxo-1,2-dihydro-1,8-naphthyridin-3-yl)oxy)-N-methylacetamide

A mixture of 2-((6-((2,5-dichloropyrimidin-4-yl)amino)-1-methyl-2-oxo-1,2-dihydro-1,8-naphthyridin-3-yl)oxy)-N-methylacetamide (52.5 mg, 128 μmol) and 3-(1-methyl-7-(4-(((R)-2-(trifluoromethyl)piperazin-1-yl)methyl)piperidin-1-yl)-1H-indazol-3-yl)piperidine-2,6-dione trifluoroacetate (93.0 mg, 153 μmol) in DMSO (0.900 mL) was treated with DIEA (112 μL, 641 μmol). The reaction mixture was warmed to 110° C. for 5 h. The reaction was then cooled to room temperature, filtered, and purified by prep-HPLC to afford 2-((6-((5-chloro-2-((3R)-4-((1-(3-(2,6-dioxopiperidin-3-yl)-1-methyl-1H-indazol-7-yl)piperidin-4-yl)methyl)-3-(trifluoromethyl)piperazin-1-yl)pyrimidin-4-yl)amino)-1-methyl-2-oxo-1,2-dihydro-1,8-naphthyridin-3-yl)oxy)-N-methylacetamide (54 mg).

¹H NMR (400 MHz, DMSO-d₆) δ 10.87 (s, 1H), 9.10 (s, 1H), 8.71 (d, J=2.5 Hz, 1H), 8.31 (d, J=2.5 Hz, 1H), 8.11 (s, 1H), 7.93 (d, J=4.9 Hz, 1H), 7.42-7.31 (m, 1H), 7.16 (s, 1H), 7.01 (d, J=4.7 Hz, 2H), 4.61 (s, 2H), 4.48-4.37 (m, 1H), 4.33 (dd, J=9.6, 5.1 Hz, 1H), 4.24 (s, 3H), 3.74 (s, 3H), 3.63-3.48 (m, 2H), 3.29-3.21 (m, 4H), 2.99-2.90 (m, 1H), 2.85-2.75 (m, 1H), 2.71-2.61 (m, 6H), 2.38-2.26 (m, 2H), 2.20-2.11 (m, 1H), 2.07 (s, 2H), 1.93-1.79 (m, 2H), 1.75-1.66 (m, 1H), 1.45-1.30 (m, 2H).

LC-MS (ESI): m/z=924.5 [M+H]⁺.

### Example 59. 2-((6-((5-chloro-2-(4-((3-(2,6-dioxopiperidin-3-yl)-1-methyl-1H-indazol-7-yl)oxy)piperidin-1-yl)pyrimidin-4-yl)amino)-1-methyl-2-oxo-1,2-dihydro-1,8-naphthyridin-3-yl)oxy)-N-methylacetamide (Compound 148)

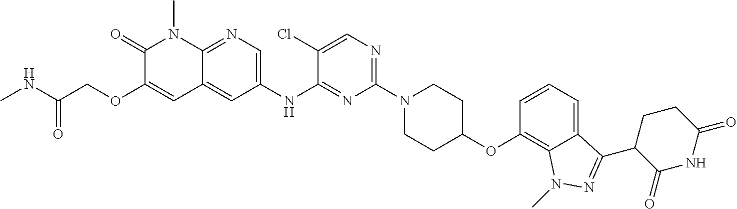 [SMILES: CNC(=O)COc1cc2cc(Nc3nc(N4CCC(Oc5cccc6c(C7CCC(=O)NC7=O)nn(C)c56)CC4)ncc3Cl)cnc2n(C)c1=O]

### Preparation of 2-((6-((5-chloro-2-(4-((3-(2,6-dioxopiperidin-3-yl)-1-methyl-1H-indazol-7-yl)oxy)piperidin-1-yl)pyrimidin-4-yl)amino)-1-methyl-2-oxo-1,2-dihydro-1,8-naphthyridin-3-yl)oxy)-N-methylacetamide

A mixture of 2-((6-((2,5-dichloropyrimidin-4-yl)amino)-1-methyl-2-oxo-1,2-dihydro-1,8-naphthyridin-3-yl)oxy)-N-methylacetamide (40.0 mg, 97.7 μmol) and 3-(1-methyl-7-(piperidin-4-yloxy)-1H-indazol-3-yl)piperidine-2,6-dione hydrochloride (44.4 mg, 117 μmol) in DMSO (0.700 mL) was treated with DIEA (68.1 μL, 391 μmol). The reaction mixture was warmed to 80° C. for 19 h. The reaction mixture was cooled to room temperature, filtered, and purified by prep-HPLC to afford 2-((6-((5-chloro-2-(4-((3-(2,6-dioxopiperidin-3-yl)-1-methyl-1H-indazol-7-yl)oxy)piperidin-1-yl)pyrimidin-4-yl)amino)-1-methyl-2-oxo-1,2-dihydro-1,8-naphthyridin-3-yl)oxy)-N-methylacetamide (28.5 mg).

¹H NMR (400 MHz, DMSO-d₆) § 10.86 (s, 1H), 9.09 (s, 1H), 8.72 (d, J=2.5 Hz, 1H), 8.29 (d, J=2.5 Hz, 1H), 8.11 (s, 1H), 7.90 (d, J=4.9 Hz, 1H), 7.21 (d, J=7.9 Hz, 1H), 7.17 (s, 1H), 7.02-6.90 (m, 2H), 4.92-4.81 (m, OH), 4.59 (s, 2H), 4.31 (dd, J=9.8, 5.1 Hz, 1H), 4.18 (s, 3H), 4.04-3.90 (m, 2H), 3.72 (s, 3H), 3.70-3.59 (m, 2H), 2.73-2.60 (m, 2H), 2.59 (d, J=4.7 Hz, 3H), 2.39-2.24 (m, 1H), 2.23-2.10 (m, 1H), 2.08-1.95 (m, 2H), 1.85-1.71 (m, 2H).

LC-MS (ESI): m/z=715.4 [M+H]⁺.

### Example 60. 2-((6-((5-chloro-2-(4-((2-(3-(2,6-dioxopiperidin-3-yl)-1-methyl-1H-indazol-7-yl)-2-azaspiro[3.5]nonan-7-yl)methyl)piperazin-1-yl)pyrimidin-4-yl)amino)-1-methyl-2-oxo-1,2-dihydro-1,8-naphthyridin-3-yl)oxy)-N-methylacetamide (Compound 149)

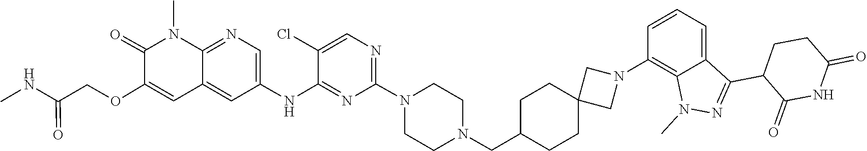 [SMILES: CNC(=O)COc1cc2cc(Nc3nc(N4CCN(CC5CCC6(CC5)CN(c5cccc7c(C8CCC(=O)NC8=O)nn(C)c57)C6)CC4)ncc3Cl)cnc2n(C)c1=O]

### Preparation of benzyl 4-((2-(3-(2,6-bis(benzyloxy)pyridin-3-yl)-1-methyl-1H-indazol-7-yl)-2-azaspiro[3.5]nonan-7-yl)methyl)piperazine-1-carboxylate

To a stirred solution of benzyl 4-((2-azaspiro[3.5]nonan-7-yl)methyl)piperazine-1-carboxylate (0.50 g, 1.39 mmol) in 1,4-dioxane:DMF (1:1) was added 3-(2,6-bis(benzyloxy)pyridin-3-yl)-7-bromo-1-methyl-1H-indazole (0.70 g, 1.39 mmol) and Cs₂CO₃ (1.36 g, 4.19 mmol). The reaction was degassed with argon for 5 minutes, followed by the addition of RuPhos (0.13 g, 0.28 mmol) and RuPhos PdG3 (0.058 g, 0.07 mmol). The resulting reaction mixture was stirred and heated at 110° C. for 3 h. The reaction mixture was poured into ice-cold water (10 mL) and extracted with ethyl acetate (2× 50 mL). The organic layers were dried over anhydrous Na₂SO₄, filtered, and dried under vacuum to obtain a crude product. The crude product was purified by flash column chromatography (46% ethyl acetate in petroleum ether) to afford benzyl 4-((2-(3-(2,6-bis(benzyloxy)pyridin-3-yl)-1-methyl-1H-indazol-7-yl)-2-azaspiro[3.5]nonan-7-yl)methyl)piperazine-1-carboxylate as a pale brown semi solid (469 mg).

LC-MS (ESI): m/z=777.42 [M+H]⁺.

### Preparation of 3-(1-methyl-7-(7-(piperazin-1-ylmethyl)-2-azaspiro[3.5]nonan-2-yl)-1H-indazol-3-yl)piperidine-2,6-dione

To a stirred solution of benzyl 4-((2-(3-(2,6-bis(benzyloxy)pyridin-3-yl)-1-methyl-1H-indazol-7-yl)-2-azaspiro[3.5]nonan-7-yl)methyl)piperazine-1-carboxylate (0.32 g, 0.41 mmol) in THE, were added MeOH, acetic acid, DMF, and 20% Pd(OH)₂ on carbon (0.58 g, 4.18 mmol) under a nitrogen atmosphere. The resultant reaction mixture was stirred at room temperature under a hydrogen atmosphere for 20 h. The reaction mixture was diluted with DCM (20 mL), filtered through a pad of celite, and washed with an excess of 30% THF in DCM (600 mL). The filtrate was collected and concentrated under reduced pressure to afford 3-(1-methyl-7-(7-(piperazin-1-ylmethyl)-2-azaspiro[3.5]nonan-2-yl)-1H-indazol-3-yl)piperidine-2,6-dione as a pale brown semi-solid (166 mg).

LC-MS (ESI): m/z=465.57 [M+H]⁺.

### Preparation of 2-((6-((5-chloro-2-(4-((2-(3-(2,6-dioxopiperidin-3-yl)-1-methyl-1H-indazol-7-yl)-2-azaspiro[3.5]nonan-7-yl)methyl)piperazin-1-yl)pyrimidin-4-yl)amino)-1-methyl-2-oxo-1,2-dihydro-1,8-naphthyridin-3-yl)oxy)-N-methylacetamide

A mixture of 2-((6-((2,5-dichloropyrimidin-4-yl)amino)-1-methyl-2-oxo-1,2-dihydro-1,8-naphthyridin-3-yl)oxy)-N-methylacetamide (40.0 mg, 97.7 μmol) and 3-(1-methyl-7-(7-(piperazin-1-ylmethyl)-2-azaspiro[3.5]nonan-2-yl)-1H-indazol-3-yl)piperidine-2,6-dione hydrochloride (58.8 mg, 117 μmol) in DMSO (0.700 mL) was treated with DIEA (50.5 mg, 68.1 μL, 391 μmol). The reaction mixture was warmed to 80° C. for 19 h. The reaction mixture was cooled to room temperature, filtered, and purified by prep-HPLC to afford 2-((6-((5-chloro-2-(4-((2-(3-(2,6-dioxopiperidin-3-yl)-1-methyl-1H-indazol-7-yl)-2-azaspiro[3.5]nonan-7-yl)methyl)piperazin-1-yl)pyrimidin-4-yl)amino)-1-methyl-2-oxo-1,2-dihydro-1,8-naphthyridin-3-yl)oxy)-N-methylacetamide (20.6 mg).

¹H NMR (400 MHz, DMSO-d₆) δ 10.87 (s, 1H), 9.55 (s, 1H), 8.70 (d, J=2.4 Hz, 1H), 8.27 (d, J=2.5 Hz, 1H), 8.18 (s, 1H), 7.95 (d, J=5.3 Hz, 1H), 7.33-7.12 (m, 2H), 6.98 (t, J=7.7 Hz, 1H), 6.73 (d, J=7.4 Hz, 1H), 4.62 (s, 2H), 4.50-4.36 (m, 1H), 4.31 (dd, J=9.5, 5.1 Hz, 1H), 4.17 (s, 3H), 3.74 (s, 3H), 3.69-3.48 (m, 7H), 3.43-3.34 (m, 1H), 3.10-2.91 (m, 4H), 2.67 (d, J=4.6 Hz, 3H), 2.64-2.58 (m, 1H), 2.41-2.25 (m, 3H), 2.22-2.06 (m, 2H), 2.07-1.91 (m, 2H), 1.90-1.80 (m, 1H), 1.81-1.68 (m, 2H), 1.65-1.42 (m, 2H), 1.17-1.04 (m, 1H). LC-MS (ESI): m/z=837.6 [M+H]⁺.

### Example 61: 2-((6-((5-chloro-2-(4-((4-(3-(2,6-dioxopiperidin-3-yl)-1-methyl-1H-indazol-7-yl)piperidin-1-yl)methyl)-4-fluoropiperidin-1-yl)pyrimidin-4-yl)amino)-1-isopropyl-2-oxo-1,2-dihydro-1,8-naphthyridin-3-yl)oxy)-N-methylacetamide (Compound 161)

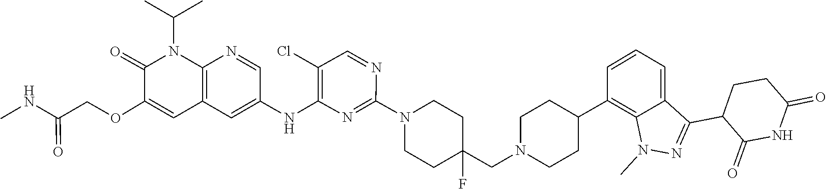 [SMILES: CNC(=O)COc1cc2cc(Nc3nc(N4CCC(F)(CN5CCC(c6cccc7c(C8CCC(=O)NC8=O)nn(C)c67)CC5)CC4)ncc3Cl)cnc2n(C(C)C)c1=O]

### Preparation of tert-butyl 4-(3-(2,6-bis(benzyloxy)pyridin-3-yl)-1-methyl-1H-indazol-7-yl)-3,6-dihydropyridine-1 (2H)-carboxylate

A mixture of tert-butyl 4-(4,4,5,5-tetramethyl-1,3,2-dioxaborolan-2-yl)-3,6-dihydropyridine-1(2H)-carboxylate (1.55 g, 5.00 mmol), 3-(2,6-bis(benzyloxy)pyridin-3-yl)-7-bromo-1-methyl-1H-indazole (2.50 g, 5.00 mmol), and cesium carbonate (4.89 g, 15.0 mmol) in 1,4-dioxane (20 mL) and water (5.0 mL) was treated with xantphos (289 mg, 500 μmol) and Pd₂(dba)₃ (229 mg, 250 μmol). The reaction mixture was equipped with a condenser and nitrogen inlet. The reaction mixture was stirred under nitrogen atmosphere at 100° C. in a heated reaction block overnight. The reaction mixture was allowed to cool to room temperature and was diluted with water. The mixture was extracted with EtOAc (3×), and the combined organics were dried over sodium sulfate, filtered, and concentrated under reduced pressure. The residue was purified by flash chromatography (20-100% EtOAc in heptanes) to afford tert-butyl 4-(3-(2,6-bis(benzyloxy)pyridin-3-yl)-1-methyl-1H-indazol-7-yl)-3,6-dihydropyridine-1 (2H)-carboxylate (2.70 g, 90%) as a yellow foam.

LC-MS (ESI): m/z=603.3 [M+H]⁺.

### Preparation of tert-butyl 4-(3-(2,6-dioxopiperidin-3-yl)-1-methyl-1H-indazol-7-yl)piperidine-1-carboxylate

A solution of tert-butyl 4-(3-(2,6-bis(benzyloxy)pyridin-3-yl)-1-methyl-1H-indazol-7-yl)-3,6-dihydropyridine-1 (2H)-carboxylate (2.70 g, 4.48 mmol) in THF (30 mL) was treated with palladium hydroxide on carbon (786 mg, 20 wt %, 1.12 mmol). Hydrogen gas (balloon) was bubbled through the solution for several minutes, and the reaction mixture was stirred at 50° C. under hydrogen atmosphere in a heated reaction block for 24 hours. The reaction mixture was removed from heat and was filtered through a pad of celite. The filtrate was taken up in DMSO and filtered through a 0.45 μm hydrophilic PTFE syringe filter. The filtrate was purified by reverse phase column chromatography (10-100% MeCN (w/0.1% formic acid) in water (w/0.1% formic acid) to afford tert-butyl 4-(3-(2,6-dioxopiperidin-3-yl)-1-methyl-1H-indazol-7-yl)piperidine-1-carboxylate (0.946 g, 50%).

LC-MS (ESI): m/z=427.3 [M+H]⁺.

### Preparation of 3-(1-methyl-7-(piperidin-4-yl)-1H-indazol-3-yl)piperidine-2,6-dione

A suspension of tert-butyl 4-(3-(2,6-dioxopiperidin-3-yl)-1-methyl-1H-indazol-7-yl)piperidine-1-carboxylate (0.946 g, 2.22 mmol) in 1,4-dioxane (10 mL) was treated with HCl (4 M in dioxane, 5.54 mL, 22.2 mmol). The reaction mixture was stirred at room temperature overnight. The reaction mixture was concentrated under reduced pressure to give crude 3-(1-methyl-7-(piperidin-4-yl)-1H-indazol-3-yl)piperidine-2,6-dione, HCl (899 mg, 99%, 98% purity) as a white solid, which was used in the next step without purification.

LC-MS (ESI): m/z=327.1 [M+H]⁺.

### Preparation of tert-butyl 4-((4-(3-(2,6-dioxopiperidin-3-yl)-1-methyl-1H-indazol-7-yl)piperidin-1-yl)methyl)-4-fluoropiperidine-1-carboxylate

A mixture of 3-(1-methyl-7-(piperidin-4-yl)-1H-indazol-3-yl)piperidine-2,6-dione, HCl (639 mg, 98 wt %, 1.57 mmol), DIPEA (546 μL, 3.14 mmol), and tert-butyl 4-fluoro-4-formylpiperidine-1-carboxylate (363 mg, 1.57 mmol) in THF (20 mL) was treated with acetic acid (269 μL, 4.70 mmol) and sodium triacetoxyborohydride (499 mg, 2.35 mmol). The reaction mixture was stirred at room temperature overnight. The reaction mixture was diluted with water and extracted with EtOAc (3×). The combined organic layers were dried over sodium sulfate, filtered, and concentrated under reduced pressure. The residue was taken up in DMSO and filtered through a 0.45 μm hydrophilic PTFE filter. The filtrate was purified by preparative HPLC to afford tert-butyl 4-((4-(3-(2,6-dioxopiperidin-3-yl)-1-methyl-1H-indazol-7-yl)piperidin-1-yl)methyl)-4-fluoropiperidine-1-carboxylate (0.299 g, 35%) as a pale yellow foam.

LC-MS (ESI): m/z=542.3 [M+H]⁺.

### Preparation of 3-(7-(1-((4-fluoropiperidin-4-yl)methyl)piperidin-4-yl)-1-methyl-1H-indazol-3-yl)piperidine-2,6-dione

A solution of tert-butyl 4-((4-(3-(2,6-dioxopiperidin-3-yl)-1-methyl-1H-indazol-7-yl)piperidin-1-yl)methyl)-4-fluoropiperidine-1-carboxylate (0.299 g, 552 μmol) in DCM (5 mL) was treated with TFA (851 μL, 11.0 mmol). The reaction mixture was stirred at room temperature overnight. The resulting reaction mixture was concentrated under reduced pressure. The residue was taken up in methanol and then concentrated under reduced pressure again to give 3-(7-(1-((4-fluoropiperidin-4-yl)methyl)piperidin-4-yl)-1-methyl-1H-indazol-3-yl)piperidine-2,6-dione, TFA (306 mg) as a pale orange semi-solid, which was used in the next step without purification.

LC-MS (ESI): m/z=442.3 [M+H]⁺.

### Preparation of 2-((6-((5-chloro-2-(4-((4-(3-(2,6-dioxopiperidin-3-yl)-1-methyl-1H-indazol-7-yl)piperidin-1-yl)methyl)-4-fluoropiperidin-1-yl)pyrimidin-4-yl)amino)-1-isopropyl-2-oxo-1,2-dihydro-1,8-naphthyridin-3-yl)oxy)-N-methylacetamide

A mixture of 2-((6-((2,5-dichloropyrimidin-4-yl)amino)-1-isopropyl-2-oxo-1,2-dihydro-1,8-naphthyridin-3-yl)oxy)-N-methylacetamide (120 mg, 275 μmol), 3-(7-(1-((4-fluoropiperidin-4-yl)methyl)piperidin-4-yl)-1-methyl-1H-indazol-3-yl)piperidine-2,6-dione, TFA (153 mg, 275 μmol), and DIPEA (240 μL, 1.38 mmol) in DMSO (1.8 mL) was stirred at 80° C. in a heated reaction block overnight. The reaction mixture was filtered through a 0.45 μm hydrophilic PTFE syringe filter. The filtrate was purified by preparative HPLC to afford 2-((6-((5-chloro-2-(4-((4-(3-(2,6-dioxopiperidin-3-yl)-1-methyl-1H-indazol-7-yl)piperidin-1-yl)methyl)-4-fluoropiperidin-1-yl)pyrimidin-4-yl)amino)-1-isopropyl-2-oxo-1,2-dihydro-1,8-naphthyridin-3-yl)oxy)-N-methylacetamide, formic acid (90.7 mg, 37%) as a tan solid.

¹H NMR (400 MHz, DMSO-d₆) δ 10.87 (s, 1H), 9.08 (s, 1H), 8.67 (d, J=2.5 Hz, 1H), 8.31 (d, J=2.6 Hz, 1H), 8.10 (s, 1H), 7.94 (d, J=4.9 Hz, 1H), 7.61-7.48 (m, 1H), 7.24 (d, J=7.1 Hz, 1H), 7.17-7.01 (m, 2H), 5.96 (s, 1H), 4.58 (s, 2H), 4.34 (dd, J=9.8, 5.1 Hz, 1H), 4.19 (s, 4H), 3.23 (d, J=11.3 Hz, 1H), 3.04 (d, J=10.9 Hz, 2H), 2.73-2.58 (m, 5H), 2.33 (dq, J=11.4, 3.9 Hz, 3H), 2.24-2.10 (m, 1H), 1.98-1.62 (m, 6H), 1.57 (d, J=6.9 Hz, 6H).

LC-MS (ESI): m/z=842.5 [M+H]⁺.

### Example 62: 2-((6-((5-chloro-2-(4-(((3S)-4-(3-(2,6-dioxopiperidin-3-yl)-1-methyl-1H-indazol-7-yl)-3-methylpiperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide (Compound 169a)

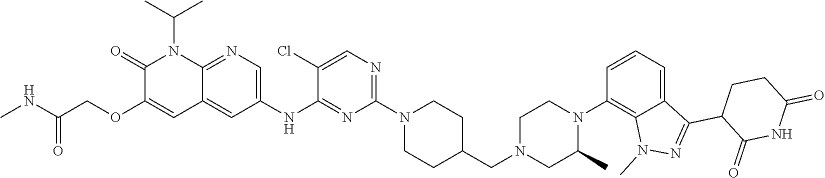 [SMILES: CNC(=O)COc1cc2cc(Nc3nc(N4CCC(CN5CCN(c6cccc7c(C8CCC(=O)NC8=O)nn(C)c67)[C@@H](C)C5)CC4)ncc3Cl)cnc2n(C(C)C)c1=O]

### Preparation of tert-butyl(S)-4-(3-(2,6-bis(benzyloxy)pyridin-3-yl)-1-methyl-1H-indazol-7-yl)-3-methylpiperazine-1-carboxylate

A stirred solution of 3-(2,6-bis(benzyloxy)pyridin-3-yl)-7-bromo-1-methyl-1H-indazole (3.00 g, 5.99 mmol), tert-butyl(S)-3-methylpiperazine-1-carboxylate (3.60 g, 17.99 mmol), and cesium carbonate (5.86 g, 17.99 mmol) in dioxane (30 mL) was degassed with argon for 5 minutes, followed by the addition of Pd-PEPPSI-iHeptCl (0.29 g, 0.30 mmol). The resulting mixture was heated to 100° C. and stirred for 18 h. The reaction mixture was filtered through pad of celite and washed with ethyl acetate (500 mL). The collected filtrate was concentrated under vacuum to obtain a crude product which was purified by flash chromatography (10%-15% of ethyl acetate in petroleum ether) to afford tert-butyl(S)-4-(3-(2,6-bis(benzyloxy)pyridin-3-yl)-1-methyl-1H-indazol-7-yl)-3-methylpiperazine-1-carboxylate (1.80 g) as a pale yellow semi-solid.

LC-MS (ESI): m/z=620.55 [M+H]⁺

### Preparation of(S)-6-(benzyloxy)-5-(1-methyl-7-(2-methylpiperazin-1-yl)-1H-indazol-3-yl)pyridin-2-ol

To a stirred solution of tert-butyl(S)-4-(3-(2,6-bis(benzyloxy)pyridin-3-yl)-1-methyl-1H-indazol-7-yl)-3-methylpiperazine-1-carboxylate (1.80 g, 2.90 mmol) in DCM (18 mL) was added TFA (14.40 mL) at 0° C. The mixture was stirred at room temperature for 2 h. The reaction mixture was concentrated under reduced pressure to afford(S)-6-(benzyloxy)-5-(1-methyl-7-(2-methylpiperazin-1-yl)-1H-indazol-3-yl)pyridin-2-ol (1.82 g) as a pale brown semi-solid which was used in the next step without purification.

LC-MS (ESI): m/z=430.29 [M+H]⁺

### Preparation of benzyl(S)-4-((4-(3-(2-(benzyloxy)-6-hydroxypyridin-3-yl)-1-methyl-1H-indazol-7-yl)-3-methylpiperazin-1-yl)methyl)piperidine-1-carboxylate

To a stirred solution of(S)-6-(benzyloxy)-5-(1-methyl-7-(2-methylpiperazin-1-yl)-1H-indazol-3-yl)pyridin-2-ol (1.82 g, 4.24 mmol) and benzyl 4-formylpiperidine-1-carboxylate (1.26 g, 5.08 mmol), acetic acid (1.82 mL), and sodium acetate (1.04 g, 12.71 mmol) in THF (72 mL) was added portion-wise sodium triacetoxyborohydride (1.80 g, 8.47 mmol). The mixture was stirred at 0° C. for 1 h then at room temperature for 16 h. The reaction mixture was quenched with water (200 mL) and extracted with ethyl acetate (500 mL). The combined organic layers were washed with brine (50 mL), dried over anhydrous Na₂SO₄, filtered, and concentrated under vacuum to obtain crude product which was purified by flash chromatography (80-90% ethyl acetate in petroleum ether) to afford benzyl(S)-4-((4-(3-(2-(benzyloxy)-6-hydroxypyridin-3-yl)-1-methyl-1H-indazol-7-yl)-3-methylpiperazin-1-yl)methyl)piperidine-1-carboxylate (0.68 g) as a pale yellow semi-solid.

LC-MS (ESI): m/z=661.41 [M+H]⁺

### Preparation of 3-(1-methyl-7-((S)-2-methyl-4-(piperidin-4-ylmethyl)piperazin-1-yl)-1H-indazol-3-yl)piperidine-2,6-dione

To a stirred solution of benzyl(S)-4-((4-(3-(2-(benzyloxy)-6-hydroxypyridin-3-yl)-1-methyl-1H-indazol-7-yl)-3-methylpiperazin-1-yl)methyl)piperidine-1-carboxylate (0.65 g, 0.98 mmol) in THF (52 mL) was added 20% Pd(OH)₂ on carbon (1.25 g, 7.87 mmol). The mixture was stirred under hydrogen atmosphere (80 psi) at room temperature for 18 h. The reaction mixture was diluted with DCM (100 mL), filtered through a celite bed, and washed with 30% THF in DCM (700 mL). The collected filtrate was concentrated under vacuum to afford 3-(1-methyl-7-((S)-2-methyl-4-(piperidin-4-ylmethyl)piperazin-1-yl)-1H-indazol-3-yl)piperidine-2,6-dione (0.42 g) as a pale brown semi-solid which was used in the next step without further purification.

LC-MS (ESI): m/z=439.32 [M+H]⁺

### Preparation of 2-((6-((5-chloro-2-(4-(((3S)-4-(3-(2,6-dioxopiperidin-3-yl)-1-methyl-1H-indazol-7-yl)-3-methylpiperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide

To a stirred solution of 3-(1-methyl-7-((S)-2-methyl-4-(piperidin-4-ylmethyl)piperazin-1-yl)-1H-indazol-3-yl)piperidine-2,6-dione (0.21 g, 0.48 mmol), 2-((6-((2,5-dichloropyrimidin-4-yl)amino)-1-isopropyl-2-oxo-1,2-dihydro-1,8-naphthyridin-3-yl)oxy)-N-methylacetamide (0.08 g, 0.19 mmol) in DMSO (2.1 mL), was added DIPEA (2.1 mL). The mixture was heated to 100° C. and stirred for 18 h. The reaction mixture was quenched with ice cold water (100 mL), and the precipitated solid was filtered and dried under vacuum to obtain crude product which was purified by prep-HPLC to afford 2-((6-((5-chloro-2-(4-(((3S)-4-(3-(2,6-dioxopiperidin-3-yl)-1-methyl-1H-indazol-7-yl)-3-methylpiperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide (0.022 g) as an off white solid.

¹H NMR: (400 MHz, DMSO-d₆): δ 10.90 (s, 1H), 9.61 (s, 1H), 9.12 (s, 1H), 8.72 (d, J=2.40 Hz, 1H), 8.27 (d, J=2.00 Hz, 1H), 8.11 (s, 1H), 7.99 (d, J=4.80 Hz, 1H), 7.57 (dd, J=2.80, 8.00 Hz, 1H), 7.25 (d, J=7.20 Hz, 1H), 7.13-7.16 (m, 2H), 5.93-5.96 (m, 1H), 4.60 (s, 1H), 4.47 (d, J=12.80 Hz, 2H), 4.35-4.37 (m, 1H), 4.28 (s, 3H), 3.56-3.60 (m, 2H), 3.04-3.11 (m, 6H), 2.89-2.92 (m, 4H), 2.64 (d, J=5.60 Hz, 3H), 2.33-2.37 (m, 2H), 2.18-2.20 (m, 2H), 1.58 (d, J=7.20 Hz, 6H), 1.36 (s, 2H), 1.24-1.22 (m, 2H), 0.88-0.86 (m, 3H).

LC-MS (ESI): m/z=839.51 [M+H]⁺

### Example 63: 2-((6-((5-chloro-2-(4-(((3R)-4-(3-(2,6-dioxopiperidin-3-yl)-1-methyl-1H-indazol-7-yl)-3-methylpiperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide (Compound 169b)

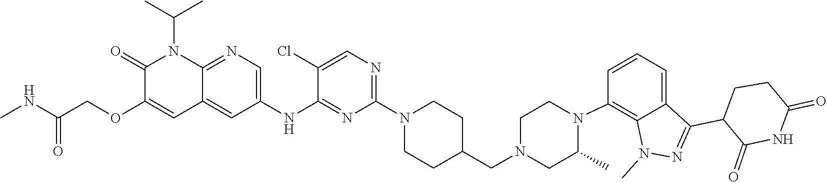 [SMILES: CNC(=O)COc1cc2cc(Nc3nc(N4CCC(CN5CCN(c6cccc7c(C8CCC(=O)NC8=O)nn(C)c67)[C@H](C)C5)CC4)ncc3Cl)cnc2n(C(C)C)c1=O]

### Preparation of tert-butyl (R)-4-(3-(2,6-bis(benzyloxy)pyridin-3-yl)-1-methyl-1H-indazol-7-yl)-3-methylpiperazine-1-carboxylate

To a stirred solution of 3-(2,6-bis(benzyloxy)pyridin-3-yl)-7-bromo-1-methyl-1H-indazole (2.0 g, 3.99 mmol) and tert-butyl (R)-3-methylpiperazine-1-carboxylate (3.19 g, 15.96 mmol) in 1,4-dioxane (40 mL) was added cesium carbonate (3.89 g, 11.97 mmol). The mixture was degassed with argon for 10 minutes followed by the addition of Pd-PEPPSI-iHeptCl (0.19 g, 0.19 mmol). The reaction mixture was stirred at 100° C. for 16 h. The reaction mixture was cooled to room temperature, filtered through a celite bed, and washed with ethyl acetate (100 mL). The filtrate was concentrated to get a crude product which was purified by flash column chromatography (15% ethyl acetate in petroleum ether) tert-butyl (R)-4-(3-(2,6-bis(benzyloxy)pyridin-3-yl)-1-methyl-1H-indazol-7-yl)-3-methylpiperazine-1-carboxylate (0.62 g) as a pale-yellow solid.

LC-MS (ESI): m/z=620.38 [M+H]⁺

### Preparation of (R)-6-(benzyloxy)-5-(1-methyl-7-(2-methylpiperazin-1-yl)-1H-indazol-3-yl)pyridin-2-ol

A stirred solution of tert-butyl (R)-4-(3-(2,6-bis(benzyloxy)pyridin-3-yl)-1-methyl-1H-indazol-7-yl)-3-methylpiperazine-1-carboxylate (0.62 g, 1.0 mmol)) in DCM (12.4 mL) was cooled to 0° C. followed by the addition of TFA (6.2 mL). The reaction mixture was stirred at room temperature for 2 h. The reaction mixture was concentrated under reduced pressure and co-distilled with diethyl ether. The crude product was washed with n-pentane to obtain (R)-6-(benzyloxy)-5-(1-methyl-7-(2-methylpiperazin-1-yl)-1H-indazol-3-yl)pyridin-2-ol (0.40 g) as a pink semi-solid.

LC-MS (ESI): m/z=430.48 [M+H]⁺

### Preparation of benzyl (R)-4-((4-(3-(2-(benzyloxy)-6-hydroxypyridin-3-yl)-1-methyl-1H-indazol-7-yl)-3-methylpiperazin-1-yl)methyl)piperidine-1-carboxylate

To a stirred solution of (R)-6-(benzyloxy)-5-(1-methyl-7-(2-methylpiperazin-1-yl)-1H-indazol-3-yl)pyridin-2-ol (0.4 g, 0.93 mmol) and benzyl 4-formylpiperidine-1-carboxylate (0.18 g, 0.74 mmol) in THF (12 mL) was added acetic acid (0.4 mL) and sodium acetate (0.22 g, 2.79 mmol) at 0° C. The mixture was stirred for 2 h followed by the addition of NaBH(OAc)₃ (0.59 g, 2.79 mmol). The mixture was stirred at room temperature for 16 h. The reaction mixture was quenched with ice-cold water (10 mL) and extracted with DCM (3×10 mL). The combined organic layers were washed with brine (10 mL), dried over anhydrous Na₂SO₄, filtered, and dried under vacuum to get a crude product which was purified by column chromatography (10% methanol in DCM) to afford benzyl (R)-4-((4-(3-(2-(benzyloxy)-6-hydroxypyridin-3-yl)-1-methyl-1H-indazol-7-yl)-3-methylpiperazin-1-yl)methyl)piperidine-1-carboxylate (0.28 g) as a pink semi-solid.

LC-MS (ESI): m/z=661.63 [M+H]⁺

### Preparation of 3-(1-methyl-7-((R)-2-methyl-4-(piperidin-4-ylmethyl)piperazin-1-yl)-1H-indazol-3-yl)piperidine-2,6-dione

To a stirred solution of benzyl (R)-4-((4-(3-(2-(benzyloxy)-6-hydroxypyridin-3-yl)-1-methyl-1H-indazol-7-yl)-3-methylpiperazin-1-yl)methyl)piperidine-1-carboxylate (0.28 g, 0.42 mmol) in THF (5.6 mL) was added 20% Pd(OH)₂, (0.35 g, 2.54 mmol). The mixture was stirred under a hydrogen atmosphere (80 psi) for 16 h at room temperature. The reaction mixture was diluted with THF and filtered through a pad of celite and washed with excess 20% THF in DCM. The filtrate was collected and concentrated to afford 3-(1-methyl-7-((R)-2-methyl-4-(piperidin-4-ylmethyl)piperazin-1-yl)-1H-indazol-3-yl)piperidine-2,6-dione as a brown semi-solid (0.26 g) which was directly used in the next step without purification.

LC-MS (ESI): m/z=439.51 [M+H]⁺

### Preparation of 2-((6-((5-chloro-2-(4-(((3R)-4-(3-(2,6-dioxopiperidin-3-yl)-1-methyl-1H-indazol-7-yl)-3-methylpiperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide

To a stirred solution of 3-(1-methyl-7-((R)-2-methyl-4-(piperidin-4-ylmethyl)piperazin-1-yl)-1H-indazol-3-yl)piperidine-2,6-dione (0.13 g,0.29 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.038 g, 0.08 mmol) in DMSO (1.3 mL) was added N,N-diisopropylethylamine (0.60 mL, 2.32 mmol). The mixture was stirred at 100° C. for 6 h. The reaction mixture was quenched with ice-cold water, filtered and dried under vacuum to get a crude product which was purified by prep-HPLC to afford 2-((6-((5-chloro-2-(4-(((3R)-4-(3-(2,6-dioxopiperidin-3-yl)-1-methyl-1H-indazol-7-yl)-3-methylpiperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide (0.017 g) as an off-white solid.

¹H NMR (400 MHz, DMSO-d₆): δ 10.88 (s, 1H), 9.04 (s, 1H), 8.69 (s, 1H), 8.46 (s, 1H), 8.30 (s, 1H), 8.07 (s, 1H), 7.96 (d, J=4.80 Hz, 1H), 7.46 (s, 1H), 7.24 (s, 1H), 7.09 (t, J=6.40 Hz, 1H), 5.96 (s, 1H), 4.58 (s, 2H), 4.47 (d, J=10.40 Hz, 2H), 4.36-4.32 (m, 1H), 4.26 (s, 3H), 3.34 (s, 1H), 3.17-3.04 (m, 2H), 3.01-2.75 (m, 4H), 2.69-2.63 (m, 5H), 2.33-2.20 (m, 5H), 1.90-1.57 (m, 4H), 1.58-1.57 (m, 6H), 1.07-1.06 (m, 2H), 1.04 (s, 3H).

LC-MS (ESI): m/z=839.52 [M+H]⁺

### Example 64: 2-((6-((5-chloro-2-((3S,4S)-4-((4-(3-(2,6-dioxopiperidin-3-yl)-1-methyl-1H-indazol-6-yl)piperazin-1-yl)methyl)-3-methylpiperidin-1-yl)pyrimidin-4-yl)amino)-1-isopropyl-2-oxo-1,2-dihydro-1,8-naphthyridin-3-yl)oxy)-N-methylacetamide (Compound 176a)

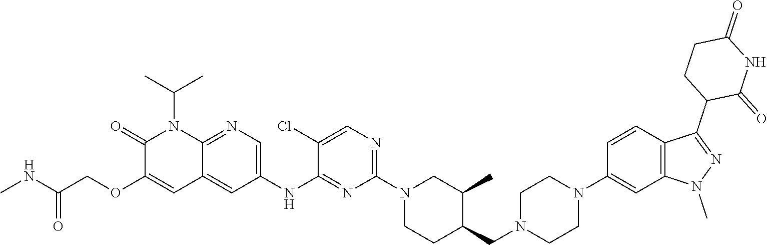 [SMILES: CNC(=O)COc1cc2cc(Nc3nc(N4CC[C@H](CN5CCN(c6ccc7c(C8CCC(=O)NC8=O)nn(C)c7c6)CC5)[C@H](C)C4)ncc3Cl)cnc2n(C(C)C)c1=O]

### Preparation of 3-(2,6-bis(benzyloxy)pyridin-3-yl)-1-methyl-6-(piperazin-1-yl)-1H-indazole

A solution of 3-(2,6-bis(benzyloxy)pyridin-3-yl)-6-bromo-1-methyl-1H-indazole (1.0 g, 1.99 mmol), piperazine (0.17 g, 1.99 mmol), and cesium carbonate (1.95 g, 5.99 mmol) in dioxane (20.0 mL) was purged with argon for 15 min. Afterwards, Ruphos (0.09 g, 0.20 mmol) and RuPhos Pd G3 (0.08 g, 0.10 mmol) was added. The mixture was heated to 100° C. and stirred for 16 h. The reaction mixture was filtered through a pad of celite and washed with ethyl acetate (200 mL). The collected filtrate was concentrated under reduced pressure to obtain a crude product which was purified by flash column chromatography (15-20% ethyl acetate in petroleum ether) to afford 3-(2,6-bis(benzyloxy)pyridin-3-yl)-1-methyl-6-(piperazin-1-yl)-1H-indazole (0.51 g) as a pale brown-solid.

LC-MS (ESI): m/z=606.75 [M+H]⁺

### Preparation of tert-butyl (3S,4S)-4-(4-(3-(2,6-bis(benzyloxy)pyridin-3-yl)-1-methyl-1H-indazol-6-yl)piperazine-1-carbonyl)-3-methylpiperidine-1-carboxylate

To a stirred solution of 3-(2,6-bis(benzyloxy)pyridin-3-yl)-1-methyl-6-(piperazin-1-yl)-1H-indazole (0.45 g, 0.02 mmol), (3S,4S)-1-(tert-butoxycarbonyl)-3-methylpiperidine-4-carboxylic acid (0.22 g, 0.02 mmol), and DIPEA (1.10 mL, 6.23 mmol) in DMF (4.50 mL) was added HATU (0.50 g, 1.33 mmol). The mixture was stirred at room temperature for 1 h. The reaction mixture was diluted with water (10 mL), and the precipitated solid was filtered and dried under reduced pressure to obtain a crude product which was triturated with diethyl ether (20 mL) and dried under vacuum to afford tert-butyl (3S,4S)-4-(4-(3-(2,6-bis(benzyloxy)pyridin-3-yl)-1-methyl-1H-indazol-6-yl)piperazine-1-carbonyl)-3-methylpiperidine-1-carboxylate (0.51 g) as an off-white solid.

LC-MS (ESI): m/z=731.46 [M+H]⁺

### Preparation of tert-butyl (3S,4S)-4-((4-(3-(2,6-bis(benzyloxy)pyridin-3-yl)-1-methyl-1H-indazol-6-yl)piperazin-1-yl)methyl)-3-methylpiperidine-1-carboxylate

To a stirred solution of tert-butyl (3S,4S)-4-(4-(3-(2,6-bis(benzyloxy)pyridin-3-yl)-1-methyl-1H-indazol-6-yl)piperazine-1-carbonyl)-3-methylpiperidine-1-carboxylate (0.45 g, 0.02 mmol) in THF (5.0 mL) was added BH₃-DMS (Neat) (2.5 mL) at 0° C. The mixture was stirred at room temperature for 7 h. The reaction mixture was quenched with methanol (100 mL) and concentrated under reduced pressure to obtain a crude product which was purified by flash column chromatography (20% ethyl acetate in petroleum ether as eluent) to afford tert-butyl (3S,4S)-4-((4-(3-(2,6-bis(benzyloxy)pyridin-3-yl)-1-methyl-1H-indazol-6-yl)piperazin-1-yl)methyl)-3-methylpiperidine-1-carboxylate (0.45 g) as an off-white solid.

LC-MS (ESI): m/z=717.53 [M+H]⁺

### Preparation of tert-butyl (3S,4S)-4-((4-(3-(2,6-dioxopiperidin-3-yl)-1-methyl-1H-indazol-6-yl)piperazin-1-yl)methyl)-3-methylpiperidine-1-carboxylate

To a stirred solution of tert-butyl (3S,4S)-4-((4-(3-(2,6-bis(benzyloxy)pyridin-3-yl)-1-methyl-1H-indazol-6-yl)piperazin-1-yl)methyl)-3-methylpiperidine-1-carboxylate (0.36 g, 0.50 mmol) in THF (108.0 mL) was added 20% palladium hydroxide (1.08 g). The mixture was stirred under hydrogen atmosphere (Parr, 80 psi) at room temperature for 18 h. The reaction was diluted with THF (200 mL), filtered through a pad of celite, and washed with excess of 40% THF in DCM (800 mL). The collected filtrate was concentrated under vacuum to obtain a crude product which was triturated with n-pentane (50 mL) and dried under vacuum to afford tert-butyl (3S,4S)-4-((4-(3-(2,6-dioxopiperidin-3-yl)-1-methyl-1H-indazol-6-yl)piperazin-1-yl)methyl)-3-methylpiperidine-1-carboxylate (0.22 g) as a pale brown semi-solid.

LC-MS (ESI): m/z=539.67 [M+H]⁺

### Preparation of 3-(1-methyl-6-(4-(((3S,4S)-3-methylpiperidin-4-yl)methyl)piperazin-1-yl)-1H-indazol-3-yl)piperidine-2,6-dione

To a stirred solution of tert-butyl (3S,4S)-4-((4-(3-(2,6-dioxopiperidin-3-yl)-1-methyl-1H-indazol-6-yl)piperazin-1-yl)methyl)-3-methylpiperidine-1-carboxylate (0.22 g, 0.41 mmol) in DCM (6.60 mL) was added TFA (2.20 mL). The mixture was stirred at room temperature for 2 h. The reaction mixture was concentrated under vacuum to obtain a crude product which was triturated with DEE (10 mL). The precipitated solid was filtered and concentrated under vacuum to afford 3-(1-methyl-6-(4-(((3S,4S)-3-methylpiperidin-4-yl)methyl)piperazin-1-yl)-1H-indazol-3-yl)piperidine-2,6-dione (0.17 g, TFA salt) as a pale brown semi-solid.

LC-MS (ESI): m/z=439.61 [M+H]⁺

### Preparation of 2-((6-((5-chloro-2-((3S,4S)-4-((4-(3-(2,6-dioxopiperidin-3-yl)-1-methyl-1H-indazol-6-yl)piperazin-1-yl)methyl)-3-methylpiperidin-1-yl)pyrimidin-4-yl) amino)-1-isopropyl-2-oxo-1,2-dihydro-1,8-naphthyridin-3-yl)oxy)-N-methylacetamide

To stirred solution of 3-(1-methyl-6-(4-(((3S,4S)-3-methylpiperidin-4-yl)methyl)piperazin-1-yl)-1H-indazol-3-yl)piperidine-2,6-dione (0.20 g, 0.45 mmol), 2-((6-((2,5-dichloropyrimidin-4-yl)amino)-1-isopropyl-2-oxo-1,2-dihydro-1,8-naphthyridin-3-yl)oxy)-N-methylacetamide (0.06 g, 0.14 mmol) in DMSO (4.0 mL) was added DIPEA (2.0 mL). The mixture was heated to 100° C. and stirred for 16 h. The reaction mixture was poured into ice-cold water (20 mL), and the precipitated solid was filtered and dried under vacuum to obtain a crude product which was purified by prep-HPLC to afford 2-((6-((5-chloro-2-((3S,4S)-4-((4-(3-(2,6-dioxopiperidin-3-yl)-1-methyl-1H-indazol-6-yl)piperazin-1-yl)methyl)-3-methylpiperidin-1-yl)pyrimidin-4-yl)amino)-1-isopropyl-2-oxo-1,2-dihydro-1,8-naphthyridin-3-yl)oxy)-N-methylacetamide (0.049 g) as an off-white solid.

LC-MS (ESI): m/z=837.46 [M−H]⁻

¹H-NMR (400 MHz, DMSO-d₆): δ 10.85 (s, 1H), 9.00 (s, 1H), 8.69 (d, J=2.40 Hz, 1H), 8.28 (s, 1H), 8.05 (s, 1H), 7.95 (d, J=4.80 Hz, 1H), 7.49 (d, J=8.80 Hz, 1H), 7.08 (s, 1H), 6.91 (d, J=9.20 Hz, 1H), 6.84 (s, 1H), 5.95 (s, 1H), 4.57 (s, 2H), 4.25-4.24 (m, 2H), 3.89 (s, 3H), 3.21 (br s, 4H), 3.09-3.06 (m, 1H), 2.94-2.90 (m, 1H), 2.67 (d, J=4.40 Hz, 3H), 2.61-2.59 (m, 6H), 2.29-2.28 (m, 4H), 2.21-1.91 (m, 2H), 1.56 (d, J=6.80 Hz, 6H), 1.46-1.41 (m, 3H), 0.92-0.89 (m, 2H).

### Example 65: 2-((6-((5-chloro-2-((3R,4S)-4-((4-(3-(2,6-dioxopiperidin-3-yl)-1-methyl-1H-indazol-6-yl)piperazin-1-yl)methyl)-3-methylpiperidin-1-yl)pyrimidin-4-yl)amino)-1-isopropyl-2-oxo-1,2-dihydro-1,8-naphthyridin-3-yl)oxy)-N-methylacetamide (Compound 176b)

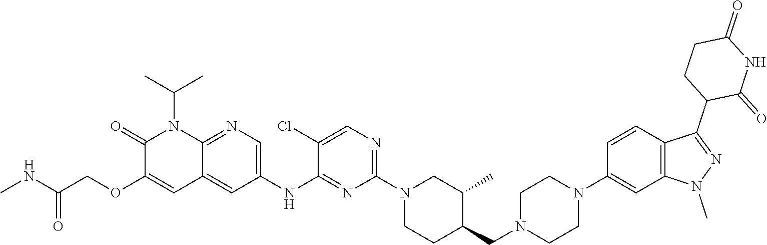 [SMILES: CNC(=O)COc1cc2cc(Nc3nc(N4CC[C@H](CN5CCN(c6ccc7c(C8CCC(=O)NC8=O)nn(C)c7c6)CC5)[C@@H](C)C4)ncc3Cl)cnc2n(C(C)C)c1=O]

### Preparation of tert-butyl (3R,4S)-4-(4-(3-(2,6-bis(benzyloxy)pyridin-3-yl)-1-methyl-1H-indazol-6-yl)piperazine-1-carbonyl)-3-methylpiperidine-1-carboxylate

To a stirred solution of (3R,4S)-1-(tert-butoxycarbonyl)-3-methylpiperidine-4-carboxylic acid (0.45 g, 1.85 mmol) and 3-(2,6-bis(benzyloxy)pyridin-3-yl)-1-methyl-6-(piperazin-1-yl)-1H-indazole (0.93 g, 1.85 mmol) in DMF (4.5 mL) was added DIPEA (2.5 mL, 14.8 mmol) and HATU (1.0 g, 2.77 mmol). The reaction mixture was stirred at room temperature for 2 h. After completion, the reaction mixture was diluted with ethyl acetate (50 mL) and washed with cold water (3×20 mL). The organic layer was dried over anhydrous sodium sulfate and concentrated under reduced pressure. The crude product was triturated by using n-pentane to afford tert-butyl (3R,4S)-4-(4-(3-(2,6-bis(benzyloxy)pyridin-3-yl)-1-methyl-1H-indazol-6-yl)piperazine-1-carbonyl)-3-methylpiperidine-1-carboxylate (1.20 g) as an off-white solid.

LC-MS (ESI): m/z=731.85 [M+H]⁺

### Preparation of tert-butyl (3R,4S)-4-((4-(3-(2,6-bis(benzyloxy)pyridin-3-yl)-1-methyl-1H-indazol-6-yl)piperazin-1-yl)methyl)-3-methylpiperidine-1-carboxylate

To a stirred solution of tert-butyl (3R,4S)-4-(4-(3-(2,6-bis(benzyloxy)pyridin-3-yl)-1-methyl-1H-indazol-6-yl)piperazine-1-carbonyl)-3-methylpiperidine-1-carboxylate (1.2 g, 1.64 mmol) in THF (24 mL) was added 2.0 M borane dimethylsulfide solution in THF (24 mL). The reaction mixture was stirred at room temperature for 8 h. The reaction mixture was quenched with methanol and evaporated under reduced pressure. The resulting residue was dissolved in methanol and refluxed at 80° C. for 2 h. The reaction mixture was concentrated under reduced pressure to get a crude product which was purified by flash column chromatography (50% ethyl acetate in petroleum ether) to afford tert-butyl (3R,4S)-4-((4-(3-(2,6-bis(benzyloxy)pyridin-3-yl)-1-methyl-1H-indazol-6-yl)piperazin-1-yl)methyl)-3-methylpiperidine-1-carboxylate (0.8 g) as a brown semi-solid.

LC-MS (ESI): m/z=717.5 [M+H]⁺

### Preparation of tert-butyl (3R,4S)-4-((4-(3-(2,6-dioxopiperidin-3-yl)-1-methyl-1H-indazol-6-yl)piperazin-1-yl)methyl)-3-methylpiperidine-1-carboxylate

To a stirred solution of tert-butyl (3R,4S)-4-((4-(3-(2,6-bis(benzyloxy)pyridin-3-yl)-1-methyl-1H-indazol-6-yl)piperazin-1-yl)methyl)-3-methylpiperidine-1-carboxylate (0.40 g, 0.55 mmol) in THF (12.0 mL) was added 20% Pd(OH)₂ on carbon (0.40 g, 100% w/w). The mixture was stirred under hydrogen atmosphere (80 psi) at room temperature for 16 h. The reaction mixture was diluted with ethyl acetate (50 mL), filtered through a celite bed, and washed with 50% THF in ethyl acetate (200 mL). The collected filtrate was concentrated under reduced pressure to get a crude product which was triturated by using diethyl ether to afford tert-butyl (3R,4S)-4-((4-(3-(2,6-dioxopiperidin-3-yl)-1-methyl-1H-indazol-6-yl)piperazin-1-yl)methyl)-3-methylpiperidine-1-carboxylate (0.30 g) as a brown semi-solid.

LC-MS (ESI): m/z=539.61 [M+H]⁺

### Preparation of 3-(1-methyl-6-(4-(((3R,4S)-3-methylpiperidin-4-yl)methyl)piperazin-1-yl)-1H-indazol-3-yl)piperidine-2,6-dione

To a stirred solution of tert-butyl (3R,4S)-4-((4-(3-(2,6-dioxopiperidin-3-yl)-1-methyl-1H-indazol-6-yl)piperazin-1-yl)methyl)-3-methylpiperidine-1-carboxylate (0.30 g, 0.56 mmol) in DCM (3.0 mL) was added TFA (1.5 mL). The mixture was stirred at room temperature for 1 h. The reaction mixture was concentrated and co-distilled with petroleum ether (2×10) under reduced pressure to get a crude product which was triturated by using diethyl ether to afford 3-(1-methyl-6-(4-(((3R,4S)-3-methylpiperidin-4-yl)methyl)piperazin-1-yl)-1H-indazol-3-yl)piperidine-2,6-dione (0.24 g) as an off-white solid.

LC-MS (ESI): m/z=439.45 [M+H]⁺

### Preparation of 2-((6-((5-chloro-2-((3R,4S)-4-((4-(3-(2,6-dioxopiperidin-3-yl)-1-methyl-1H-indazol-6-yl)piperazin-1-yl)methyl)-3-methylpiperidin-1-yl)pyrimidin-4-yl)amino)-1-isopropyl-2-oxo-1,2-dihydro-1,8-naphthyridin-3-yl)oxy)-N-methylacetamide

To a stirred solution of 3-(1-methyl-6-(4-(((3R,4S)-3-methylpiperidin-4-yl)methyl)piperazin-1-yl)-1H-indazol-3-yl)piperidine-2,6-dione (0.10 g, 0.23 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.03 g, 0.07 mmol) in DMSO (2.0 mL) was added DIPEA (0.2 mL, 1.14 mmol). The resulting reaction mixture was stirred at 100° C. for 6 h. The reaction mixture was poured into ice-cold water and stirred for 15 minutes. The resulting precipitate was filtered and dried to get crude product which was purified by prep-HPLC to afford 2-((6-((5-chloro-2-((3R,4S)-4-((4-(3-(2,6-dioxopiperidin-3-yl)-1-methyl-1H-indazol-6-yl)piperazin-1-yl)methyl)-3-methylpiperidin-1-yl)pyrimidin-4-yl)amino)-1-isopropyl-2-oxo-1,2-dihydro-1,8-naphthyridin-3-yl)oxy)-N-methylacetamide (38 mg) as an off-white solid.

LC-MS (ESI): m/z=839.51 [M+H]⁺

¹H-NMR (400 MHz, DMSO-d₆): δ 10.84 (s, 1H), 9.04 (s, 1H), 8.70 (d, J=2.40 Hz, 1H), 8.50 (s, 1H), 8.26 (d, J=2.0 Hz, 1H), 8.07 (s, 1H), 7.93 (d, J=4.40 Hz, 1H), 7.49 (d, J=9.20 Hz, 1H), 7.12 (s, 1H), 6.91 (d, J=9.20 Hz, 1H), 6.83 (s, 1H), 5.98 (s, 1H), 4.55 (s, 2H), 4.26-4.25 (m, 3H), 3.90 (s, 3H), 3.25-3.21 (m, 4H), 2.84-2.81 (m, 1H), 2.70-2.60 (m, 7H), 2.50-2.40 (m, 3H), 2.29-2.27 (m, 1H), 2.13-2.10 (m, 2H), 1.93-1.90 (m, 1H), 1.56 (d, J=6.80 Hz, 7H), 1.29-1.28 (m, 1H), 1.06-1.04 (m, 1H), 0.92 (d, J=6.0 Hz, 3H).

### Example 66: 2-((6-((5-chloro-2-((3R,4R)-4-((4-(3-(2,6-dioxopiperidin-3-yl)-1-methyl-1H-indazol-6-yl)piperazin-1-yl)methyl)-3-methylpiperidin-1-yl)pyrimidin-4-yl)amino)-1-isopropyl-2-oxo-1,2-dihydro-1,8-naphthyridin-3-yl)oxy)-N-methylacetamide (Compound 176c)

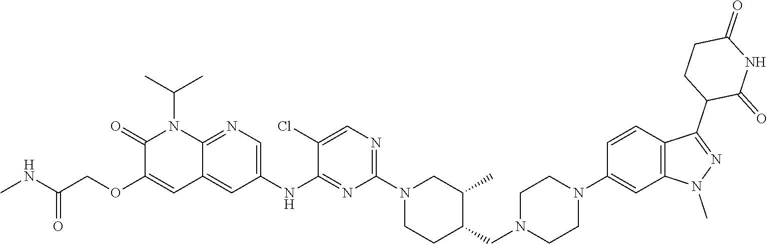 [SMILES: CNC(=O)COc1cc2cc(Nc3nc(N4CC[C@@H](CN5CCN(c6ccc7c(C8CCC(=O)NC8=O)nn(C)c7c6)CC5)[C@@H](C)C4)ncc3Cl)cnc2n(C(C)C)c1=O]

### Preparation of tert-butyl (3R,4R)-4-(4-(3-(2,6-bis(benzyloxy)pyridin-3-yl)-1-methyl-1H-indazol-6-yl)piperazine-1-carbonyl)-3-methylpiperidine-1-carboxylate

To a stirred solution of 3-(2,6-bis(benzyloxy)pyridin-3-yl)-1-methyl-6-(piperazin-1-yl)-1H-indazole (1.0 g, 1.97 mmol) and (3R,4R)-1-(tert-butoxycarbonyl)-3-methylpiperidine-4-carboxylic acid (0.48 g, 1.97 mmol) in DMF (10 mL) was added DIPEA (1.79 g, 13.84 mmol) followed by HATU (1.12 g, 2.96 mmol) at 0° C. The resultant reaction mixture was stirred at room temperature for 1 h. The reaction mixture was quenched with cold water (20 mL), and the solid was filtered and dried under reduced pressure to obtain tert-butyl (3R,4R)-4-(4-(3-(2,6-bis(benzyloxy)pyridin-3-yl)-1-methyl-1H-indazol-6-yl)piperazine-1-carbonyl)-3-methylpiperidine-1-carboxylate (1.3 g) as a white solid.

LC-MS (ESI): m/z=731.5 [M+H]⁺

### Preparation of tert-butyl (3R,4R)-4-((4-(3-(2,6-bis(benzyloxy)pyridin-3-yl)-1-methyl-1H-indazol-6-yl)piperazin-1-yl)methyl)-3-methylpiperidine-1-carboxylate

To a stirred solution of tert-butyl (3R,4R)-4-(4-(3-(2,6-bis(benzyloxy)pyridin-3-yl)-1-methyl-1H-indazol-6-yl)piperazine-1-carbonyl)-3-methylpiperidine-1-carboxylate (1.3 g, 1.77 mmol) in THF (39 mL) was added BH₃-DMS (104 mL) at 0° C. The resultant reaction mixture was stirred at room temperature for 16 h. The reaction was quenched with methanol (100 mL) slowly and concentrated under reduced pressure. The crude product was purified by flash column chromatography (20% ethyl acetate in petroleum ether) to provide tert-butyl (3R,4R)-4-((4-(3-(2,6-bis(benzyloxy)pyridin-3-yl)-1-methyl-1H-indazol-6-yl)piperazin-1-yl)methyl)-3-methylpiperidine-1-carboxylate (1.2 g) as a white semi-solid.

LC-MS (ESI): m/z=717.53 [M+H]⁺

### Preparation of tert-butyl (3R,4R)-4-((4-(3-(2,6-dioxopiperidin-3-yl)-1-methyl-1H-indazol-6-yl)piperazin-1-yl)methyl)-3-methylpiperidine-1-carboxylate

To a stirred solution of tert-butyl (3R,4R)-4-((4-(3-(2,6-bis(benzyloxy)pyridin-3-yl)-1-methyl-1H-indazol-6-yl)piperazin-1-yl)methyl)-3-methylpiperidine-1-carboxylate (1.2 g, 1.67 mmol) in THF (48 mL) under a nitrogen atmosphere was added 20% palladium hydroxide on carbon (4.69 g). The reaction mixture was stirred under a hydrogen atmosphere with 80 psi at room temperature for 16 h. The reaction mixture was filtered through a celite bed and washed with 20% THF in DCM (3×100 mL). The combined organic layer was concentered to provide a crude product which was purified by triturating with diethyl ether followed by n-pentane to afford tert-butyl (3R,4R)-4-((4-(3-(2,6-dioxopiperidin-3-yl)-1-methyl-1H-indazol-6-yl)piperazin-1-yl)methyl)-3-methylpiperidine-1-carboxylate as a brown semi-solid (0.62 g)

LC-MS (ESI): m/z=539.65 [M+H]⁺

### Preparation of 3-(1-methyl-6-(4-(((3R,4R)-3-methylpiperidin-4-yl)methyl)piperazin-1-yl)-1H-indazol-3-yl)piperidine-2,6-dione

To a stirred solution of tert-butyl (3R,4R)-4-((4-(3-(2,6-dioxopiperidin-3-yl)-1-methyl-1H-indazol-6-yl)piperazin-1-yl)methyl)-3-methylpiperidine-1-carboxylate (0.68 g, 1.26 mmol) in DCM (6.8 mL) was added trifluoroacetic acid (0.77 mL, 10.09 mmol). The mixture was stirred at room temperature for 2 h. The reaction mixture was concentrated under vacuum to get a crude compound which was triturated by diethyl ether (5 mL) to afford 3-(1-methyl-6-(4-(((3R,4R)-3-methylpiperidin-4-yl)methyl)piperazin-1-yl)-1H-indazol-3-yl)piperidine-2,6-dione (0.5 g) as a brown semi-solid.

LC-MS (ESI): m/z=439.28 [M+H]

### Preparation of 2-((6-((5-chloro-2-((3R,4R)-4-((4-(3-(2,6-dioxopiperidin-3-yl)-1-methyl-1H-indazol-6-yl)piperazin-1-yl)methyl)-3-methylpiperidin-1-yl)pyrimidin-4-yl)amino)-1-isopropyl-2-oxo-1,2-dihydro-1,8-naphthyridin-3-yl)oxy)-N-methylacetamide

To a stirred solution of 3-(1-methyl-6-(4-(((3R,4R)-3-methylpiperidin-4-yl)methyl)piperazin-1-yl)-1H-indazol-3-yl)piperidine-2,6-dione (0.15 g, 0.76 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.045 g, 0.45 mmol) in DMSO (3.0 mL) was added N,N-diisopropylethylamine (0.49 g, 6.08 mmol). The reaction mixture was stirred at 100° C. for 5 h. The reaction mixture was poured into ice cold water and stirred for 15 minutes. The resulting precipitate was filtered and dried to get crude product which was purified by prep-HPLC 2-((6-((5-chloro-2-((3R,4R)-4-((4-(3-(2,6-dioxopiperidin-3-yl)-1-methyl-1H-indazol-6-yl)piperazin-1-yl)methyl)-3-methylpiperidin-1-yl)pyrimidin-4-yl)amino)-1-isopropyl-2-oxo-1,2-dihydro-1,8-naphthyridin-3-yl)oxy)-N-methylacetamide (0.0157 g) as a pale brown solid.

¹H NMR (400 MHz, DMSO-d₆): δ 10.85 (s, 1H), 9.00 (s, 1H), 8.69 (s, 1H), 8.27 (s, 1H), 8.16 (s, 1H), 7.94 (1H), 7.49 (d, J=8.80 Hz, 1H), 7.08 (s, 1H), 6.91 (d, 1H), 6.84 (s, 1H), 5.90 (s, 1H), 4.57 (s, 2H), 4.38-4.36 (m, 1H), 4.27-4.24 (m, 2H), 3.89 (s, 3H), 3.32-3.21 (m, 4H), 3.09 (d, J=Hz, 1H), 2.94-2.64 (m, 1H), 2.62-2.50 (m, 9H), 2.31-2.13 (m, 4H), 1.96-1.91 (m, 2H), 1.57-1.39 (m, 9H), 0.77 (d, J=6.40 Hz, 3H).

LC-MS (ESI): m/z=837.43 [M−H]⁻

### Example 67: 2-((6-((5-chloro-2-((3S,4S)-4-((4-(3-(2,6-dioxopiperidin-3-yl)-1-methyl-1H-indazol-7-yl)piperazin-1-yl)methyl)-3-methylpiperidin-1-yl)pyrimidin-4-yl)amino)-1-isopropyl-2-oxo-1,2-dihydro-1,8-naphthyridin-3-yl)oxy)-N-methylacetamide (Compound 177a)

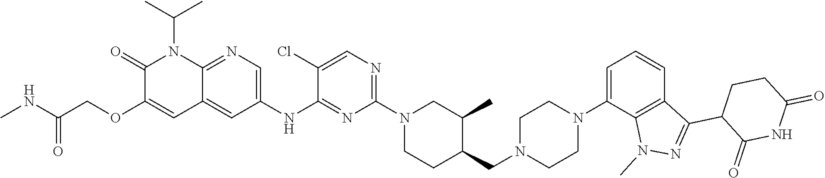 [SMILES: CNC(=O)COc1cc2cc(Nc3nc(N4CC[C@H](CN5CCN(c6cccc7c(C8CCC(=O)NC8=O)nn(C)c67)CC5)[C@H](C)C4)ncc3Cl)cnc2n(C(C)C)c1=O]

### Preparation of tert-butyl 4-(3-(2,6-bis(benzyloxy)pyridin-3-yl)-1-methyl-1H-indazol-7-yl) piperazine-1-carboxylate

A solution of 3-(2,6-bis(benzyloxy)pyridin-3-yl)-7-bromo-1-methyl-1H-indazole (1.0 g, 1.99 mmol) and piperazine (0.17 g, 2.0 mmol) and cesium carbonate (1.95 g, 5.99 mmol) in 1,4-dioxane (20.0 mL) was purged with argon for 10 minutes. Pd-PEPPSI-iHeptCl (0.10 g, 0.10 mmol) was then added and heated to 100° C. for 12 h. The reaction mixture was diluted with ethyl acetate (100 mL) and quenched with water (30 mL). The separated organic layer was dried over anhydrous Na₂SO₄, filtered, and concentrated under vacuum to obtain a crude product which was purified by flash column chromatography (20% ethyl acetate in petroleum ether) to afford tert-butyl 4-(3-(2,6-bis(benzyloxy)pyridin-3-yl)-1-methyl-1H-indazol-7-yl) piperazine-1-carboxylate (0.70 g) as a pale brown semi-solid.

LC-MS (ESI): m/z=506.55 [M+H]⁺

### Preparation of tert-butyl (3S,4S)-4-(4-(3-(2,6-bis(benzyloxy)pyridin-3-yl)-1-methyl-1H-indazol-7-yl)piperazine-1-carbonyl)-3-methylpiperidine-1-carboxylate

To a stirred solution of tert-butyl 4-(3-(2,6-bis(benzyloxy)pyridin-3-yl)-1-methyl-1H-indazol-7-yl)piperazine-1-carboxylate (0.77 g, 1.86 mmol) and (3S,4S)-1-(tert-butoxycarbonyl)-3-methylpiperidine-4-carboxylic acid (0.45 g, 1.86 mmol) in DMF (9.40 mL) was added DIPEA (2.30 mL, 13.01 mmol) followed by HATU (1.06 g, 2.79 mmol) at 0° C. The resultant reaction mixture was stirred at room temperature for 1 h. The reaction mixture was poured into ice cold water (20 mL), and the precipitated solid was filtered and dried under vacuum to obtain a crude product. The crude product was triturated with n-pentane (20 mL) to afford tert-butyl (3S,4S)-4-(4-(3-(2,6-bis(benzyloxy)pyridin-3-yl)-1-methyl-1H-indazol-7-yl)piperazine-1-carbonyl)-3-methylpiperidine-1-carboxylate (0.50 g) as an off-white solid.

LC-MS (ESI): m/z=731.73 [M+H]⁺

### Preparation of tert-butyl (3S,4S)-4-((4-(3-(2,6-bis(benzyloxy)pyridin-3-yl)-1-methyl-1H-indazol-7-yl)piperazin-1-yl)methyl)-3-methylpiperidine-1-carboxylate

To a stirred solution of tert-butyl (3S,4S)-4-(4-(3-(2,6-bis(benzyloxy)pyridin-3-yl)-1-methyl-1H-indazol-7-yl)piperazine-1-carbonyl)-3-methylpiperidine-1-carboxylate (1.0 g, 1.37 mmol) in THF (10.0 mL) was added BH₃.DMS (10.0 mL) at 0° C. The mixture was stirred at room temperature for 18 h. The reaction mixture was quenched with methanol (30 mL) and heated at 80° C. for 18 h. The reaction mixture was concentrated under vacuum to obtain a crude product which was triturated with n-pentane (20 mL) to afford tert-butyl (3S,4S)-4-((4-(3-(2,6-bis(benzyloxy)pyridin-3-yl)-1-methyl-1H-indazol-7-yl)piperazin-1-yl)methyl)-3-methylpiperidine-1-carboxylate (0.80 g) as an off-white solid.

LC-MS (ESI): m/z=717.71 [M+H]⁺

### Preparation of tert-butyl (3S,4S)-4-((4-(3-(2,6-dioxopiperidin-3-yl)-1-methyl-1H-indazol-7-yl)piperazin-1-yl)methyl)-3-methylpiperidine-1-carboxylate

To a stirred solution tert-butyl (3S,4S)-4-((4-(3-(2,6-bis(benzyloxy)pyridin-3-yl)-1-methyl-1H-indazol-7-yl)piperazin-1-yl)methyl)-3-methylpiperidine-1-carboxylate (0.54 g, 0.75 mmol) in THF (100.0 mL) was added 20% Pd(OH)₂/C (2.0 g, 400% w/w). The mixture was stirred under hydrogen atmosphere (80 psi) at room temperature for 18 h. The reaction mixture was diluted with DCM (100 mL) and filtered through a celite bed which was washed with an excess of 30% THF:DCM (500 mL). The collected filtrate was concentrated under vacuum to obtain a crude product which was triturated with n-pentane (100 mL) to afford tert-butyl (3S,4S)-4-((4-(3-(2,6-dioxopiperidin-3-yl)-1-methyl-1H-indazol-7-yl)piperazin-1-yl)methyl)-3-methylpiperidine-1-carboxylate (0.35 g) as an off-white solid.

LC-MS (ESI): m/z=539.44 [M+H]⁺

### Preparation of 3-(1-methyl-7-(4-(((3S,4S)-3-methylpiperidin-4-yl)methyl)piperazin-1-yl)-1H-indazol-3-yl)piperidine-2,6-dione

To a stirred solution tert-butyl (3S,4S)-4-((4-(3-(2,6-dioxopiperidin-3-yl)-1-methyl-1H-indazol-7-yl)piperazin-1-yl)methyl)-3-methylpiperidine-1-carboxylate (0.30 g, 0.55 mmol) in DCM (6.0 mL) was added TFA (3.0 mL) at 0° C. The mixture was stirred at room temperature for 2 h. The reaction mixture was concentrated under vacuum to obtain a crude product which was triturated with n-pentane (10 mL) to afford 3-(1-methyl-7-(4-(((3S,4S)-3-methylpiperidin-4-yl)methyl)piperazin-1-yl)-1H-indazol-3-yl)piperidine-2,6-dione (0.40 g) as a pale-brown solid.

LC-MS (ESI): m/z=439.51 [M+H]⁺

### Preparation of 2-((6-((5-chloro-2-((3S,4S)-4-((4-(3-(2,6-dioxopiperidin-3-yl)-1-methyl-1H-indazol-7-yl)piperazin-1-yl)methyl)-3-methylpiperidin-1-yl)pyrimidin-4-yl)amino)-1-isopropyl-2-oxo-1,2-dihydro-1,8-naphthyridin-3-yl)oxy)-N-methylacetamide

To a stirred solution of 3-(1-methyl-7-(4-(((3S,4S)-3-methylpiperidin-4-yl)methyl)piperazin-1-yl)-1H-indazol-3-yl)piperidine-2,6-dione (0.45 g, 1.03 mmol) and DIPEA (4.50 mL) in DMSO (9.0 mL) was added 2-((6-((2,5-dichloropyrimidin-4-yl)amino)-1-isopropyl-2-oxo-1,2-dihydro-1,8-naphthyridin-3-yl)oxy)-N-methylacetamide (0.09 g, 0.20 mmol). The mixture was stirred at 100° C. for 18 h. The reaction mixture was poured into ice-cold water (20 mL), and the precipitated solid was filtered and dried under vacuum to obtain a crude product which was purified by prep-HPLC to afford 2-((6-((5-chloro-2-((3S,4S)-4-((4-(3-(2,6-dioxopiperidin-3-yl)-1-methyl-1H-indazol-7-yl)piperazin-1-yl)methyl)-3-methylpiperidin-1-yl)pyrimidin-4-yl)amino)-1-isopropyl-2-oxo-1,2-dihydro-1,8-naphthyridin-3-yl)oxy)-N-methylacetamide (0.05 g) as an off white solid.

¹H NMR (400 MHz, DMSO-d₆): δ 10.88 (s, 1H), 9.00 (s, 1H), 8.69 (d, J=2.40 Hz, 1H), 8.28 (d, J=2.00 Hz, 1H), 8.05 (s, 1H), 7.95 (d, J=4.80 Hz, 1H), 7.39 (dd, J=1.20, 7.20 Hz, 1H), 7.08 (s, 1H), 7.02-7.00 (m, 2H), 5.95 (br s, 1H), 4.62 (s, 2H), 4.33-4.32 (m, 2H), 4.24 (s, 4H), 3.32-3.22 (m, 4H), 3.09-3.096 (m, 4H), 2.66 (d, J=8.80 Hz, 3H), 2.62-2.61 (m, 2H), 2.50-2.35 (m, 4H), 2.26-2.24 (m, 2H), 2.17-2.15 (m, 2H), 1.57 (d, J=6.80 Hz, 6H), 1.39-1.36 (m, 2H), 0.78 (d, J=6.80 Hz, 3H).

LC-MS (ESI): m/z=839.52 [M+H]⁺

### Example 68: 2-((6-((5-chloro-2-((3R,4S)-4-((4-(3-(2,6-dioxopiperidin-3-yl)-1-methyl-1H-indazol-7-yl)piperazin-1-yl)methyl)-3-methylpiperidin-1-yl)pyrimidin-4-yl)amino)-1-isopropyl-2-oxo-1,2-dihydro-1,8-naphthyridin-3-yl)oxy)-N-methylacetamide (Compound 177d)

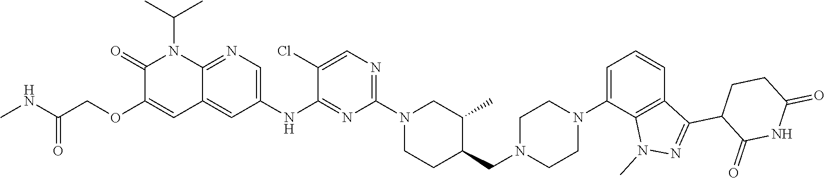 [SMILES: CNC(=O)COc1cc2cc(Nc3nc(N4CC[C@H](CN5CCN(c6cccc7c(C8CCC(=O)NC8=O)nn(C)c67)CC5)[C@@H](C)C4)ncc3Cl)cnc2n(C(C)C)c1=O]

### Preparation of 3-(2,6-bis(benzyloxy)pyridin-3-yl)-1-methyl-7-(piperazin-1-yl)-1H-indazole

To a stirred solution of 3-(2,6-bis(benzyloxy)pyridin-3-yl)-7-bromo-1-methyl-1H-indazole (5.0 g, 9.99 mmol) and piperazine (5.0 g, 19.98 mmol) in 1,4-dioxane (100 mL) was added cesium carbonate (9.76 g, 29.97 mmol). The mixture was degassed with argon for 15 minutes followed by the addition of added Pd-PEPPSI-IHeptCl (0.48 g, 0.49 mmol). The mixture was stirred at 100° C. for 16 h. The reaction mixture was diluted with ethyl acetate (50 mL), filtered through a celite bed, and washed with ethyl acetate (100 mL). The collected filtrate was concentrated under reduced pressure to get crude product which was purified by flash column chromatography (50% ethyl acetate in petroleum ether) to afford 3-(2,6-bis(benzyloxy)pyridin-3-yl)-1-methyl-7-(piperazin-1-yl)-1H-indazole (3.0 g) as a brown semi-solid.

LC-MS (ESI): m/z=506.65 [M+H]⁺

### Preparation of tert-butyl (3R,4S)-4-(4-(3-(2,6-bis(benzyloxy)pyridin-3-yl)-1-methyl-1H-indazol-7-yl)piperazine-1-carbonyl)-3-methylpiperidine-1-carboxylate

To a stirred solution of (3R,4S)-1-(tert-butoxycarbonyl)-3-methylpiperidine-4-carboxylic acid (0.48 g, 1.97 mmol) and 3-(2,6-bis(benzyloxy)pyridin-3-yl)-1-methyl-7-(piperazin-1-yl)-1H-indazole (1.0 g, 1.97 mmol) in THF (20 mL) was added DIPEA (1.37 mL, 7.88 mmol). The mixture stirred for 10 min followed by the addition of HATU (108 mg, 0.28 mmol) at 0° C. The mixture was stirred at room temperature for 2 h. After completion, the reaction mass was diluted with ethyl acetate (30 mL) and washed with cold water (3×20 mL). The organic layer was dried over anhydrous sodium sulphate and concentrated under reduced pressure. The crude was triturated by using n-pentane to afford tert-butyl (3R,4S)-4-(4-(3-(2,6-bis(benzyloxy)pyridin-3-yl)-1-methyl-1H-indazol-7-yl)piperazine-1-carbonyl)-3-methylpiperidine-1-carboxylate (1.3 g) as an off-white solid.

LC-MS (ESI): m/z=731.77 [M+H]⁺

### Preparation of tert-butyl (3R,4S)-4-((4-(3-(2,6-bis(benzyloxy)pyridin-3-yl)-1-methyl-1H-indazol-7-yl)piperazin-1-yl)methyl)-3-methylpiperidine-1-carboxylate

To a stirred solution of tert-butyl (3R,4S)-4-(4-(3-(2,6-bis(benzyloxy)pyridin-3-yl)-1-methyl-1H-indazol-7-yl)piperazine-1-carbonyl)-3-methylpiperidine-1-carboxylate (1.3 g, 1.77 mmol) in THF (26 mL) was added 2.0 M borane dimethylsulfide solution in THF (26 mL,). The reaction mixture was stirred at room temperature for 8 h. The reaction mixture was quenched with methanol and evaporated under reduced pressure. Then the residue was dissolved in methanol and refluxed at 80° C. for 2 h. The reaction mixture was concentrated under reduced pressure to get a crude product which was purified by flash column chromatography (50% ethyl acetate in petroleum ether) to afford tert-butyl (3R,4S)-4-((4-(3-(2,6-bis(benzyloxy)pyridin-3-yl)-1-methyl-1H-indazol-7-yl)piperazin-1-yl)methyl)-3-methylpiperidine-1-carboxylate (0.9 g) as a brown semi-solid.

LC-MS (ESI): m/z=717.75 [M+H]

### Preparation of tert-butyl (3R,4S)-4-((4-(3-(2,6-dioxopiperidin-3-yl)-1-methyl-1H-indazol-7-yl)piperazin-1-yl)methyl)-3-methylpiperidine-1-carboxylate

To a stirred solution of tert-butyl (3R,4S)-4-((4-(3-(2,6-bis(benzyloxy)pyridin-3-yl)-1-methyl-1H-indazol-7-yl)piperazin-1-yl)methyl)-3-methylpiperidine-1-carboxylate (0.90 g, 1.25 mmol) in THF (45.0 mL) was added 20% Pd(OH)₂ on carbon, moisture 50% wet (3.6 g). The mixture was stirred under hydrogen atmosphere (80 Psi) at room temperature for 16 h. The reaction mixture was diluted with ethyl acetate (50 mL), filtered through a celite bed, and washed with 50% THF in ethyl acetate (200 mL). The collected filtrate was concentrated under reduced pressure to get a crude product which was triturated by using diethyl ether to afford tert-butyl (3R,4S)-4-((4-(3-(2,6-dioxopiperidin-3-yl)-1-methyl-1H-indazol-7-yl)piperazin-1-yl)methyl)-3-methylpiperidine-1-carboxylate (0.60 g) as a brown semi-solid.

LC-MS (ESI): m/z=539.67 [M+H]⁺

### Preparation of 3-(1-methyl-7-(4-(((3R,4S)-3-methylpiperidin-4-yl)methyl)piperazin-1-yl)-1H-indazol-3-yl)piperidine-2,6-dione

To a stirred solution of tert-butyl (3R,4S)-4-((4-(3-(2,6-dioxopiperidin-3-yl)-1-methyl-1H-indazol-7-yl)piperazin-1-yl)methyl)-3-methylpiperidine-1-carboxylate (0.60 g, 1.11 mmol) in DCM (6.0 mL) was added TFA (6.0 mL). The mixture was stirred at room temperature for 1 h. The reaction mixture was concentrated and co-distilled with petroleum ether under reduced pressure to get a crude product which was triturated by using diethyl ether to afford 3-(1-methyl-7-(4-(((3R,4S)-3-methylpiperidin-4-yl)methyl)piperazin-1-yl)-1H-indazol-3-yl)piperidine-2,6-dione (0.4 g) as an off-white solid.

LC-MS (ESI): m/z=439.55 [M+H]⁺

### Preparation of 2-((6-((5-chloro-2-((3R,4S)-4-((4-(3-(2,6-dioxopiperidin-3-yl)-1-methyl-1H-indazol-7-yl)piperazin-1-yl)methyl)-3-methylpiperidin-1-yl)pyrimidin-4-yl)amino)-1-isopropyl-2-oxo-1,2-dihydro-1,8-naphthyridin-3-yl)oxy)-N-methylacetamide

To a stirred solution of 3-(1-methyl-7-(4-(((3R,4S)-3-methylpiperidin-4-yl)methyl)piperazin-1-yl)-1H-indazol-3-yl)piperidine-2,6-dione (0.20 g, 0.45 mmol) in DMSO (4.0 mL) was added 2-((6-((2,5-dichloropyrimidin-4-yl)amino)-1-isopropyl-2-oxo-1,2-dihydro-1,8-naphthyridin-3-yl)oxy)-N-methylacetamide (0.06 g, 0.13 mmol). The reaction mixture was stirred at 100° C. for 16 h. The reaction mixture was poured into ice-cold water and stirred for 15 minutes. The resulting precipitate was filtered and dried to get crude product which was purified by prep-HPLC to afford 2-((6-((5-chloro-2-((3R,4S)-4-((4-(3-(2,6-dioxopiperidin-3-yl)-1-methyl-1H-indazol-7-yl)piperazin-1-yl)methyl)-3-methylpiperidin-1-yl)pyrimidin-4-yl)amino)-1-isopropyl-2-oxo-1,2-dihydro-1,8-naphthyridin-3-yl)oxy)-N-methylacetamide (0.06 g) as a white solid.

LC-MS (ESI): m/z=839.48 [M+H]⁺

¹H-NMR (400 MHz, DMSO-d₆): § 10.88 (s, 1H), 9.04 (s, 1H), 8.70 (d, J=2.40 Hz, 1H), 8.26 (d, J=2.00 Hz, 1H), 8.07 (s, 1H), 7.94 (d, J=4.80 Hz, 1H), 7.38-7.36 (m, 1H), 7.13 (s, 1H), 7.01-7.00 (m, 2H), 5.98 (s, 1H), 4.56 (s, 2H), 4.351 (br, 1H), 4.33-4.31 (m, 2H), 4.24 (s, 3H), 2.84-2.81 (m, 8H), 2.67-2.64 (m, 4H), 2.59-2.58 (m, 3H), 2.33-2.32 (m, 1H), 2.15-2.13 (m, 3H), 1.92-1.91 (m, 1H), 1.57 (d, J=6.80 Hz, 6H), 1.47-1.45 (m, 1H), 1.30-1.29 (m, 1H), 1.12-1.07 (m, 1H), 0.92 (d, J=6.40 Hz, 3H).

### Example 69: 2-((6-((5-chloro-2-(7-((3-(2,6-dioxopiperidin-3-yl)-1-methyl-1H-indazol-6-yl)amino)-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide (Compound 182)

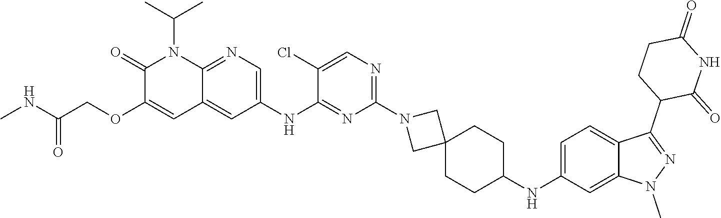 [SMILES: CNC(=O)COc1cc2cc(Nc3nc(N4CC5(CCC(Nc6ccc7c(C8CCC(=O)NC8=O)nn(C)c7c6)CC5)C4)ncc3Cl)cnc2n(C(C)C)c1=O]

### Preparation of tert-butyl 7-[[3-(2,6-dioxo-3-piperidyl)-1-methyl-indazol-6-yl]amino]-2-azaspiro[3.5]nonane-2-carboxylate

To a solution of tert-butyl 7-oxo-2-azaspiro[3.5]nonane-2-carboxylate (2 g, 8.36 mmol) and 3-(6-amino-1-methyl-indazol-3-yl)piperidine-2,6-dione (2.16 g, 8.36 mmol) in DCM (20 mL) was added NaBH(OAc)₃ (2.66 g, 12.54 mmol) and AcOH (50.19 mg, 835.74 μmol, 47.84 μL). The mixture was stirred at 0° C. for 2 h. The reaction mixture was diluted with H₂O (100 mL) and extracted with ethyl acetate (300 mL). The combined organic layers were washed with brine (100 mL), dried over Na₂SO₄, filtered, and concentrated under reduced pressure to give a residue. The residue was purified by column chromatography (petroleum ether/ethyl acetate 3/1 to 0/1) to afford tert-butyl 7-[[3-(2,6-dioxo-3-piperidyl)-1-methyl-indazol-6-yl]amino]-2-azaspiro[3.5]nonane-2-carboxylate (3.4 g, 7.06 mmol) as a yellow solid.

LC-MS (ESI): m/z=482.2 [M+H]⁺

### Preparation of 3-[6-(2-azaspiro[3.5]nonan-7-ylamino)-1-methyl-indazol-3-yl]piperidine-2,6-dione

To a solution of tert-butyl 7-[[3-(2,6-dioxo-3-piperidyl)-1-methyl-indazol-6-yl]amino]-2-azaspiro[3.5]nonane-2-carboxylate (3.4 g, 7.06 mmol) in DCM (10 mL) was added HCl/dioxane (10 mL). The mixture was stirred at 25° C. for 2 h. The mixture was concentrated under reduced pressure to give a residue. The residue was purified by column chromatography (SiO₂, DCM/MeOH=I/O to 5/1). 3-[6-(2-azaspiro[3.5]nonan-7-ylamino)-1-methyl-indazol-3-yl]piperidine-2,6-dione (1.5 g, 3.74 mmol) was obtained as a gray solid.

LC-MS (ESI): m/z=382.2 [M+H]⁺

¹H NMR (400 MHz, DMSO-d₆) δ=10.86 (s, 1H), 8.79-8.69 (m, 1H), 8.25 (s, 1H), 8.04 (s, 1H), 7.86 (d, J=7.6 Hz, 1H), 7.56 (d, J=8.8 Hz, 1H), 7.45 (d, J=8.8 Hz, 1H), 7.11-7.05 (m, 1H), 6.73 (d, J=9.2 Hz, 1H), 6.13 (s, 1H), 4.53-4.36 (m, 3H), 4.29 (dd, J=5.2, 9.2 Hz, 1H), 3.92 (s, 3H), 3.70 (d, J=8.0 Hz, 4H), 3.58 (s, 3H), 3.30 (s, 4H), 2.23-2.10 (m, 2H), 1.91 (d, J=9.6 Hz, 5H), 1.69-1.52 (m, 5H), 1.27-1.25 (m, 1H), 1.24 (s, 1H), 1.40-1.22 (m, 1H), 0.78-0.68 (m, 1H), 0.54 (t, J=5.6 Hz, 2H), 0.42-0.34 (m, 1H)

### Preparation of 2-((6-((5-chloro-2-(7-((3-(2,6-dioxopiperidin-3-yl)-1-methyl-1H-indazol-6-yl)amino)-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide

To a solution of 2-((6-((2,5-dichloropyrimidin-4-yl)amino)-1-isopropyl-2-oxo-1,2-dihydro-1,8-naphthyridin-3-yl)oxy)-N-methylacetamide (50 mg, 114.34 μmol) in DMSO (1 mL) was added triethylamine (46.28 mg, 457.37 μmol, 63.66 μmol) and 3-[6-(2-azaspiro[3.5]nonan-7-ylamino)-1-methyl-indazol-3-yl]piperidine-2,6-dione (52.34 mg, 137.21 μmol). The mixture was stirred at 100° C. for 1 hr. The reaction mixture was diluted with H₂O (20 mL), and then the mixture was extracted with ethyl acetate (60 mL). The combined organic layers were washed with brine (10 mL×2), dried over Na₂SO₄, filtered, and concentrated under reduced pressure to give a residue. The residue was purified by prep-TLC (DCM:MeOH=20:1) to afford 2-((6-((5-chloro-2-(7-((3-(2,6-dioxopiperidin-3-yl)-1-methyl-1H-indazol-6-yl)amino)-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide (17.6 mg, 20.70 μmol) as a brown solid.

LC-MS (ESI): m/z=782 [M+H]⁺

¹H NMR (400 MHz, DMSO-d₆) δ=10.81 (s, 1H), 9.02 (s, 1H), 8.78 (s, 1H), 8.47 (s, 1H), 8.11-7.93 (m, 2H), 7.32-7.30 (m, 1H), 7.14 (s, 1H), 6.53-6.51 (m, 1H), 6.38 (s, 1H), 6.06-5.88 (m, 1H), 5.76-5.67 (m, 1H), 4.60 (s, 2H), 4.18-4.16 (m, 1H), 4.07-3.96 (m, 1H), 3.83-3.68 (m, 6H), 2.74-2.57 (m, 6H), 2.34-2.11 (m, 2H), 1.99-1.88 (m, 4H), 1.70-1.54 (m, 7H), 1.33-1.15 (m, 3H)

### Example 70: 2-[[6-[[5-chloro-2-[7-[3-(2,6-dioxo-3-piperidyl)-1-methyl-indazol-6-yl]oxy-2-azaspiro[3.5]nonan-2-yl]pyrimidin-4-yl]amino]-1-isopropyl-2-oxo-1,8-naphthyridin-3-yl]oxy]-N-methyl-acetamide (Compound 183)

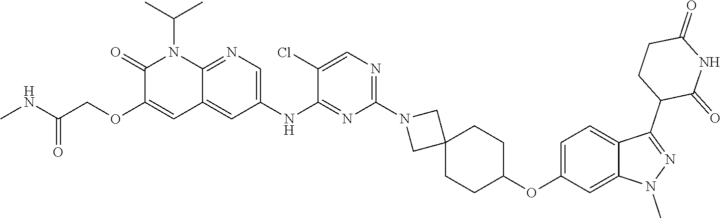 [SMILES: CNC(=O)COc1cc2cc(Nc3nc(N4CC5(CCC(Oc6ccc7c(C8CCC(=O)NC8=O)nn(C)c7c6)CC5)C4)ncc3Cl)cnc2n(C(C)C)c1=O]

### Preparation of tert-butyl 7-[3-(2,6-dibenzyloxy-3-pyridyl)-1-methyl-indazol-6-yl]oxy-2-azaspiro[3.5]nonane-2-carboxylate

A mixture of tert-butyl 7-hydroxy-2-azaspiro[3.5]nonane-2-carboxylate (2 g, 8.29 mmol) and 3-(2,6-dibenzyloxy-3-pyridyl)-1-methyl-indazol-6-ol (3.99 g, 9.12 mmol) in toluene (20 mL) was degassed and purged with N₂ three times. To this mixture was added 2-(tributyl-phosphanylidene) acetonitrile (2.00 g, 8.29 mmol). The mixture was stirred at 120° C. for 12 h under N₂ atmosphere. The reaction mixture was filtered and concentrated under reduced pressure to give a residue. The residue was purified by column chromatography (petroleum ether/ethyl acetate=3/1 to 0/1) to afford tert-butyl 7-[3-(2,6-dibenzyloxy-3-pyridyl)-1-methyl-indazol-6-yl]oxy-2-azaspiro[3.5]nonane-2-carboxylate (2.1 g, 3.18 mmol) as a white solid.

LC-MS (ESI): m/z=661.4 [M+H]⁺

### Preparation of tert-butyl 7-[3-(2,6-dioxo-3-piperidyl)-1-methyl-indazol-6-yl]oxy-2-azaspiro[3.5]nonane-2-carboxylate

A solution of tert-butyl 7-[3-(2,6-dibenzyloxy-3-pyridyl)-1-methyl-indazol-6-yl]oxy-2-azaspiro[3.5]nonane-2-carboxylate (2.1 g, 2.86 mmol) in 2,2,2-trifluoroethanol (45 mL) and AcOH (17.18 mg, 286.02 μmol, 16.37 μL) was degassed and purged with N₂ three times. To this mixture was added Pd/C (760.95 mg, 715.04 μmol, 10% purity) under N₂ atmosphere. The mixture was purged with H₂ three times and then stirred at 25° C. for 6 h. The mixture was concentrated under reduced pressure to give a residue. The residue was purified by column chromatography (petroleum ether/ethyl acetate=1/0 to 1/1) to afford tert-butyl 7-[3-(2,6-dioxo-3-piperidyl)-1-methyl-indazol-6-yl]oxy-2-azaspiro[3.5]nonane-2-carboxylate (1 g, 2.07 mmol) as a yellow oil.

### Preparation of 3-[6-(2-azaspiro[3.5]nonan-7-yloxy)-1-methyl-indazol-3-yl]piperidine-2,6-dione

To a solution of tert-butyl 7-[3-(2,6-dioxo-3-piperidyl)-1-methyl-indazol-6-yl]oxy-2-azaspiro[3.5]nonane-2-carboxylate (3.4 g, 7.06 mmol) in DCM (10 mL) was added HCl/dioxane (10 mL). The mixture was stirred at 25° C. for 2 hr. The mixture was concentrated under reduced pressure to give a residue. The residue was purified by column chromatography (DCM/MeOH=I/O to 5/1) to afford 3-[6-(2-azaspiro[3.5]nonan-7-yloxy)-1-methyl-indazol-3-yl]piperidine-2,6-dione (1.5 g, 3.74 mmol) as a gray solid.

LC-MS (ESI): m/z=383.2 [M+H]⁺

¹H NMR (400 MHz, DMSO-d₆): δ=10.85 (s, 1H), 9.02 (br d, J=1.2 Hz, 2H), 7.55 (d, J=8.8 Hz, 1H), 7.07 (d, J=1.6 Hz, 1H), 6.72 (dd, J=2.0, 8.8 Hz, 1H), 4.54-4.45 (m, 1H), 4.29 (dd, J=5.2, 9.2 Hz, 1H), 3.92 (s, 3H), 3.69 (br d, J=3.6 Hz, 4H), 2.70-2.59 (m, 2H), 2.37-2.26 (m, 1H), 2.21-2.10 (m, 1H), 2.02-1.92 (m, 2H), 1.91-1.82 (m, 2H), 1.73-1.63 (m, 2H), 1.59-1.50 (m, 2H)

### Preparation of 2-[[6-[[5-chloro-2-[7-[3-(2,6-dioxo-3-piperidyl)-1-methyl-indazol-6-yl]oxy-2-azaspiro[3.5]nonan-2-yl]pyrimidin-4-yl]amino]-1-isopropyl-2-oxo-1,8-naphthyridin-3-yl]oxy]-N-methyl-acetamide

To a solution of 2-[[6-[(2,5-dichloropyrimidin-4-yl)amino]-1-isopropyl-2-oxo-1,8-naphthyridin-3-yl]oxy]-N-methyl-acetamide (300 mg, 686.06 μmol) in DMSO (1 mL) was added 3-[6-(2-azaspiro[3.5]nonan-7-yloxy)-1-methyl-indazol-3-yl]piperidine-2,6-dione (314.86 mg, 823.27 μmol) and N,N-diethylethanamine (277.69 mg, 2.74 mmol, 381.96 μL). The mixture was stirred at 100° C. for 1 hour. The reaction mixture was diluted with water (50 mL) and extracted with ethyl acetate (30 mL×2). The combined organic layers were washed with brine (50 mL×2), dried over Na₂SO₄, filtered, and concentrated under reduced pressure to give a residue. The residue was purified by column chromatography (DCM:MeOH=5/1 to 1/1) to afford 2-[[6-[[5-chloro-2-[7-[3-(2,6-dioxo-3-piperidyl)-1-methyl-indazol-6-yl]oxy-2-azaspiro[3.5]nonan-2-yl]pyrimidin-4-yl]amino]-1-isopropyl-2-oxo-1,8-naphthyridin-3-yl]oxy]-N-methyl-acetamide (400 mg, 480.04 μmol) as a white solid.

LC-MS (ESI): m/z=783.7 [M+H]⁺

¹H NMR (400 MHz, DMSO-d₆) δ=10.85 (s, 1H), 9.02 (s, 1H), 8.77 (s, 1H), 8.42 (d, J=1.6 Hz, 1H), 8.06 (s, 1H), 7.96 (d, J=4.8 Hz, 1H), 7.55 (d, J=8.8 Hz, 1H), 7.16 (s, 1H), 7.08 (s, 1H), 6.74 (d, J=8.4 Hz, 1H), 6.03-5.91 (m, 1H), 5.75 (s, 3H), 4.61-4.48 (m, 3H), 4.29 (dd, J=5.2, 9.2 Hz, 1H), 3.92 (s, 3H), 3.72 (s, 3H), 2.70-2.59 (m, 6H), 2.34-2.28 (m, 1H), 2.20-2.10 (m, 1H), 1.99-1.90 (m, 3H), 1.72-1.65 (m, 2H), 1.57 (d, J=6.8 Hz, 6H).

### Example 71: 2-[[6-[[5-chloro-2-[7-[[3-(2,6-dioxo-3-piperidyl)-5-fluoro-1-methyl-indazol-6-yl]amino]-2-azaspiro[3.5]nonan-2-yl]pyrimidin-4-yl]amino]-1-isopropyl-2-oxo-1,8-naphthyridin-3-yl]oxy]-N-methyl-acetamide (Compound 184)

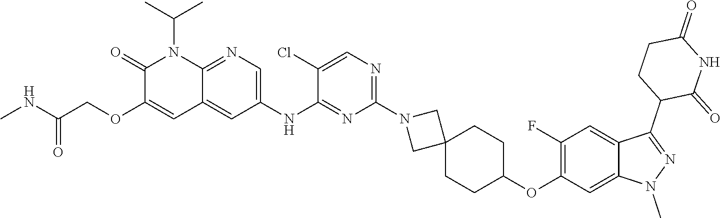 [SMILES: CNC(=O)COc1cc2cc(Nc3nc(N4CC5(CCC(Oc6cc7c(cc6F)c(C6CCC(=O)NC6=O)nn7C)CC5)C4)ncc3Cl)cnc2n(C(C)C)c1=O]

### Preparation of 6-bromo-5-fluoro-3-iodo-1H-indazole

To a solution of 6-bromo-5-fluoro-1H-indazole (20 g, 93.01 mmol) in dioxane (200 mL) and H₂O (60 mL) was added KOH (7.83 g, 139.52 mmol, 46.51 mL) and I₂ (35.41 g, 139.52 mmol, 28.10 mL). The mixture was stirred at 25° C. for 2 h. The reaction mixture was concentrated under reduced pressure to remove dioxane. The residue was diluted with H₂O (2000 mL) and extracted with ethyl acetate (2000 mL), dried over Na₂SO₄, filtered, and concentrated under reduced pressure to give 6-bromo-5-fluoro-3-iodo-1H-indazole (31 g, 90.93 mmol) as a brown solid, which was used in the next step without further purification.

¹H NMR (400 MHz, DMSO-d₆) δ=8.00 (d, J=5.6 Hz, 1H), 7.37 (d, J=8.4 Hz, 1H).

### Preparation of 6-bromo-5-fluoro-3-iodo-1-methyl-indazole

A mixture of 6-bromo-5-fluoro-3-iodo-1H-indazole (25 g, 73.33 mmol), Mel (20.82 g, 146.66 mmol, 9.13 mL), and NaH (4.40 g, 110.00 mmol) in DMF (250 mL) was degassed and purged with N₂ three times at 0° C. The mixture was stirred at 25° C. for 1 hr under N₂ atmosphere. The reaction mixture was quenched by addition of NH₄Cl (40 mL) at 0° C., diluted with H₂O (300 mL), and extracted with ethyl acetate (900 mL). The combined organic layers were washed with NaCl (900 mL), dried over Na₂SO₄, filtered, and concentrated under reduced pressure to give a residue. The residue was purified by flash silica gel chromatography (0~20% ethyl acetate/petroleum ether) to give 6-bromo-5-fluoro-3-iodo-1-methyl-indazole (15 g, 40.99 mmol) as a white solid.

LC-MS (ESI): m/z=354.9 [M+H]⁺

### Preparation of 6-bromo-3-(2,6-dibenzyloxy-3-pyridyl)-5-fluoro-1-methyl-indazole

A mixture of 6-bromo-5-fluoro-3-iodo-1-methyl-indazole (15 g, 42.26 mmol), 2,6-dibenzyloxy-3-(4,4,5,5-tetramethyl-1,3,2-dioxaborolan-2-yl)pyridine (17.64 g, 42.26 mmol), Pd(dppf)Cl₂ (3.09 g, 4.23 mmol), and Cs₂CO₃ (41.31 g, 126.78 mmol) in THF (150 mL) was degassed and purged with N₂ three times. The mixture was stirred at 60° C. for 12 h under N₂ atmosphere. The reaction mixture was partitioned between H₂O (200 mL) and ethyl acetate (600 mL). The organic phase was separated, dried over Na₂SO₄, filtered, and concentrated under reduced pressure to give a residue. The residue was purified by flash silica gel chromatography (0~15% ethyl acetate/petroleum ether) to afford 6-bromo-3-(2,6-dibenzyloxy-3-pyridyl)-5-fluoro-1-methyl-indazole (9.5 g, 17.96 mmol) as a brown solid.

LC-MS (ESI): m/z=518.1 [M+H]⁺.

### Preparation of tert-butyl 7-amino-2-azaspiro[3.5]nonane-2-carboxylate

A mixture of tert-butyl 7-oxo-2-azaspiro[3.5]nonane-2-carboxylate (14 g, 58.50 mmol) NH₄OAc (18.04 g, 234.01 mmol), NaBH₄ (4.43 g, 117.00 mmol) in iPrOH (150 mL) at −20° C. was degassed and purged with N₂ three times. The mixture was stirred at 20° C. for 1 h under N₂ atmosphere. The reaction mixture was quenched by addition of NH₄Cl (60 mL) at 0° C., and then diluted with H₂O (300 mL). The mixture was extracted with ethyl acetate (900 mL), dried over Na₂SO₄, filtered, and concentrated under reduced pressure to give a residue. The crude product was triturated with DCM at 10° C. for 30 min to afford tert-butyl 7-amino-2-azaspiro[3.5]nonane-2-carboxylate (14 g, 58.25 mmol) as a white solid.

### Preparation of tert-butyl 7-[[3-(2,6-dibenzyloxy-3-pyridyl)-5-fluoro-1-methyl-indazol-6-yl]amino]-2-azaspiro[3.5]nonane-2-carboxylate

A mixture of 6-bromo-3-(2,6-dibenzyloxy-3-pyridyl)-5-fluoro-1-methyl-indazole (9.5 g, 18.33 mmol), tert-butyl 7-amino-2-azaspiro[3.5]nonane-2-carboxylate (6.61 g, 27.49 mmol), tBuONa (3.70 g, 38.49 mmol), BINAP (2.28 g, 3.67 mmol), and Pd₂(dba)₃ (1.68 g, 1.83 mmol) in dioxane (100 mL) was degassed and purged with N₂ three times. The mixture was stirred at 95° C. for 5 h under N₂ atmosphere. The reaction mixture was partitioned between H₂O (400 mL) and ethyl acetate (1200 mL). The organic phase was separated, washed with ethyl acetate (1200 mL), dried over Na₂SO₄, filtered, and concentrated under reduced pressure to give a residue. The residue was purified by flash silica gel chromatography (0~48% ethyl acetate/petroleum ether) to afford tert-butyl 7-[[3-(2,6-dibenzyloxy-3-pyridyl)-5-fluoro-1-methyl-indazol-6-yl]amino]-2-azaspiro[3.5]nonane-2-carboxylate (10 g, 12.54 mmol) as a brown solid

LC-MS (ESI): m/z=678.3 [M+H]⁺

### Preparation of tert-butyl 7-[[3-(2,6-dioxo-3-piperidyl)-5-fluoro-1-methyl-indazol-6-yl]amino]-2-azaspiro[3.5]nonane-2-carboxylate

A mixture of tert-butyl 7-[[3-(2,6-dibenzyloxy-3-pyridyl)-5-fluoro-1-methyl-indazol-6-yl]amino]-2-azaspiro[3.5]nonane-2-carboxylate (9.5 g, 14.02 mmol), Pd/C (14.92 g, 14.02 mmol, 10% purity), Pd(OH)₂ (9.84 g, 14.02 mmol, 20% purity) and AcOH (2.52 g, 42.05 mmol, 2.41 mL) in CF₃CH₂OH (100 mL) was degassed and purged with H₂ three times. The mixture was stirred at 25° C. for 16 h under H₂ atmosphere. The mixture was concentrated under reduced pressure to give a residue. The residue was purified by flash silica gel chromatography (0~70% ethyl acetate/petroleum ether) to afford tert-butyl 7-[[3-(2,6-dioxo-3-piperidyl)-5-fluoro-1-methyl-indazol-6-yl]amino]-2-azaspiro[3.5]nonane-2-carboxylate (6 g, 11.53 mmol) as a brown oil.

LC-MS (ESI): m/z=500.2 [M+H]⁺

### Preparation of 3-[6-(2-azaspiro[3.5]nonan-7-ylamino)-5-fluoro-1-methyl-indazol-3-yl]piperidine-2,6-dione

A solution of tert-butyl 7-[[3-(2,6-dioxo-3-piperidyl)-5-fluoro-1-methyl-indazol-6-yl]amino]-2-azaspiro[3.5]nonane-2-carboxylate (6 g, 12.01 mmol) in TFA (6 mL) and DCM (18 mL) was stirred at 25° C. for 0.5 h. The mixture was concentrated under reduced pressure to give 3-[6-(2-azaspiro[3.5]nonan-7-ylamino)-5-fluoro-1-methyl-indazol-3-yl]piperidine-2,6-dione (4 g, 9.31 mol) as a brown oil.

LC-MS (ESI): m/z=661.3 [M+H]⁺

¹H NMR (400 MHz, MeOD-d4) δ=7.27 (d, J=11.6 Hz, 1H), 6.60 (d, J=7.2 Hz, 1H), 4.23 (dd, J=5.2, 9.2 Hz, 1H), 91 (s, 3H), 3.88 (s, 2H), 3.81 (s, 2H), 3.51-3.42 (m, 1H), 2.83-2.65 (m, 2H), 2.40 (dt, J=4.4, 9.2 Hz, 1H), 2.32-2.23 (m, H), 2.17-2.04 (m, 4H), 1.86-1.69 (m, 2H), 1.42-1.27 (m, 2H).

### Preparation of 2-[[6-[[5-chloro-2-[7-[[3-(2,6-dioxo-3-piperidyl)-5-fluoro-1-methyl-indazol-6-yl]amino]-2-azaspiro[3.5]nonan-2-yl]pyrimidin-4-yl]amino]-1-isopropyl-2-oxo-1,8-naphthyridin-3-yl]oxy]-N-methyl-acetamide

To a solution of 2-[[6-[(2,5-dichloropyrimidin-4-yl)amino]-1-isopropyl-2-oxo-1,8-naphthyridin-3-yl]oxy]-N-methylacetamide (50 mg, 114.34 μmol) and 3-[6-(2-azaspiro[3.5]nonan-7-ylamino)-5-fluoro-1-methyl-indazol-3-yl]piperidine-2,6-dione (54.81 mg, 137.21 μmol) in DMSO (1 mL) was added Et₃N (46.28 mg, 457.37 μmol, 63.66 μL). The mixture was stirred at 100° C. for 2 h. The reaction mixture was filtered, and the filtrate was concentrated to give a residue. The residue was purified by prep-HPLC to afford 2-[[6-[[5-chloro-2-[7-[[3-(2,6-dioxo-3-piperidyl)-5-fluoro-1-methyl-indazol-6-yl]amino]-2-azaspiro[3.5]nonan-2-yl]pyrimidin-4-yl]amino]-1-isopropyl-2-oxo-1,8-naphthyridin-3-yl]oxy]-N-methyl-acetamide (13.71 mg, 16.05 μmol) as a yellow solid.

LC-MS (ESI): m/z=800.3 [M+H]⁺.

¹H NMR (400 MHz, DMSO-d₆) δ=10.81 (s, 1H), 9.02 (s, 1H), 8.78 (s, 1H), 8.58-8.43 (m, 1H), 8.07 (s, 1H), 8.03-7.92 (m, 1H), 7.31 (d, J=12.0 Hz, 1H), 7.11 (s, 1H), 6.66 (d, J=7.2 Hz, 1H), 6.04-5.88 (m, 1H), 5.40-5.25 (m, 1H), 4.60 (s, 2H), 4.18 (dd, J=5.6, 8.8 Hz, 1H), 3.86 (s, 3H), 3.78-3.62 (m, 4H), 3.48-3.38 (m, 1H), 2.67 (s, 3H), 2.18-2.10 (m, 1H), 1.94 (d, J=10.4 Hz, 5H), 1.67 (t, J=11.2 Hz, 3H), 1.57 (d, J=6.8 Hz, 6H), 1.49-1.32 (m, 3H).

### Example 72: 2-[[6-[[5-chloro-2-[(6R,7S)-7-[3-(2,6-dioxo-3-piperidyl)-1-methyl-indazol-6-yl]oxy-6-methyl-2-azaspiro[3.5]nonan-2-yl]pyrimidin-4-yl]amino]-1-isopropyl-2-oxo-1,8-naphthyridin-3-yl]oxy]-N-methyl-acetamide [rel-2-((6-((5-chloro-2-((6R,7S)-7-((3-(2,6-dioxopiperidin-3-yl)-1-methyl-1H-indazol-6-yl)oxy)-6-methyl-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide] (Compound 185a)

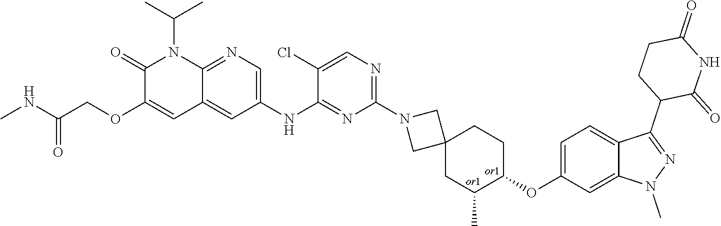 [SMILES: CNC(=O)COc1cc2cc(Nc3nc(N4CC5(CC[C@H](Oc6ccc7c(C8CCC(=O)NC8=O)nn(C)c7c6)[C@H](C)C5)C4)ncc3Cl)cnc2n(C(C)C)c1=O]

### Preparation of tert-butyl (6R,7S)-7-[3-(2,6-dibenzyloxy-3-pyridyl)-1-methyl-indazol-6-yl]oxy-6-methyl-2-azaspiro[3.5]nonane-2-carboxylate

To a solution of tert-butyl (6R,7R)-7-hydroxy-6-methyl-2-azaspiro[3.5]nonane-2-carboxylate (500 mg, 1.96 mmol) in toluene (10 mL) was added 2-(tributyl-phosphanylidene) acetonitrile (567.11 mg, 2.35 mmol) and 3-(2,6-dibenzyloxy-3-pyridyl)-1-methyl-indazol-6-ol (856.64 mg, 1.96 mmol). The mixture was stirred at 120° C. for 12 h. The reaction mixture was concentrated under reduced pressure to give a residue. The residue was purified by prep-HPLC to afford tert-butyl (6R,7S)-7-[3-(2,6-dibenzyloxy-3-pyridyl)-1-methyl-indazol-6-yl]oxy-6-methyl-2-azaspiro[3.5]nonane-2-carboxylate (320 mg, 469.45 μmol) as a yellow oil.

LC-MS (ESI): m/z=675.7 [M+H]⁺

### Preparation of tert-butyl (6R,7S)-7-[3-(2,6-dioxo-3-piperidyl)-1-methyl-indazol-6-yl]oxy-6-methyl-2-azaspiro[3.5]nonane-2-carboxylate

To a solution of tert-butyl (6R,7S)-7-[3-(2,6-dibenzyloxy-3-pyridyl)-1-methyl-indazol-6-yl]oxy-6-methyl-2-azaspiro[3.5]nonane-2-carboxylate (300 mg, 444.56 μmol) in CF₃CH₂OH (3 mL) was added Pd/C (473.10 mg, 444.56 μmol, 10% purity), Pd(OH)₂ (312.16 mg, 444.56 μmol, 20% purity), and CH₃COOH (2.67 mg, 44.46 μmol, 2.54 μL) under N₂ atmosphere. The mixture was stirred under H₂ at 25° C. for 2 h. The reaction mixture was filtered, and the filtrate was concentrated to give a residue. The residue was purified by flash silica gel chromatography (64% ethyl acetate/petroleum ether) to afford tert-butyl (6R,7S)-7-[3-(2,6-dioxo-3-piperidyl)-1-methyl-indazol-6-yl]oxy-6-methyl-2-azaspiro[3.5]nonane-2-carboxylate (200 mg, 394.69 μmol) as a yellow oil.

LC-MS (ESI): m/z=497.3 [M+H]⁺

### Preparation of 3-[1-methyl-6-[[(6R,7S)-6-methyl-2-azaspiro[3.5]nonan-7-yl]oxy] indazol-3-yl]piperidine-2,6-dione

To a solution of tert-butyl (6R,7S)-7-[3-(2,6-dioxo-3-piperidyl)-1-methyl-indazol-6-yl]oxy-6-methyl-2-azaspiro[3.5]nonane-2-carboxylate (200 mg, 402.74 μmol, 1 eq) in DCM (2.5 mL) was added HCl/dioxane (4 M, 2.00 mL). The mixture was stirred at 25° C. for 1 h. The reaction mixture was concentrated under reduced pressure to give 3-[1-methyl-6-[[(6R,7S)-6-methyl-2-azaspiro[3.5]nonan-7-yl]oxy] indazol-3-yl]piperidine-2,6-dione (170 mg, 394.47 μmol) as a white solid.

LC-MS (ESI): m/z=397.4 [M+H]⁺

¹H NMR (400 MHz, DMSO-d₆): δ=10.87 (s, 1H), 9.07 (br s, 2H), 7.55 (d, J=9.2 Hz, 1H), 7.07 (s, 1H), 6.75 (dd, J=1.6, 8.8 Hz, 1H), 5.76 (s, 1H), 4.48 (br s, 1H), 4.29 (dd, J=5.2, 9.6 Hz, 1H), 3.91 (s, 4H), 3.76-3.72 (m, 1H), 3.63 (br d, J=4.4 Hz, 1H), 2.70-2.58 (m, 2H), 2.34-2.28 (m, 1H), 2.15 (br dd, J=5.2, 13.6 Hz, 1H), 2.03-1.95 (m, 1H), 1.84-1.76 (m, 2H), 1.68-1.61 (m, 1H), 1.51-1.41 (m, 1H), 1.23 (s, 1H), 0.98 (br d, J=6.4 Hz, 3H)

### Preparation of 2-[[6-[[5-chloro-2-[(6R,7S)-7-[3-(2,6-dioxo-3-piperidyl)-1-methyl-indazol-6-yl]oxy-6-methyl-2-azaspiro[3.5]nonan-2-yl]pyrimidin-4-yl]amino]-1-isopropyl-2-oxo-1,8-naphthyridin-3-yl]oxy]-N-methyl-acetamide [rel-2-((6-((5-chloro-2-((6R,7S)-7-((3-(2,6-dioxopiperidin-3-yl)-1-methyl-1H-indazol-6-yl)oxy)-6-methyl-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide]

To a solution of 2-[[6-[(2,5-dichloropyrimidin-4-yl)amino]-1-isopropyl-2-oxo-1,8-naphthyridin-3-yl]oxy]-N-methyl-acetamide (50 mg, 114.34 μmol, 1 eq) in DMSO (1 mL) was added 3-[1-methyl-6-[[(6R,7S)-6-methyl-2-azaspiro[3.5]nonan-7-yl]oxy] indazol-3-yl]piperidine-2,6-dione (45.34 mg, 114.34 μmol) and TEA (46.28 mg, 457.37 μmol, 63.66 μL). The mixture was stirred at 100° C. for 2 h. The reaction mixture was filtered, and the filtrate was concentrated to give a residue. The residue was purified by prep-HPLC to afford 2-[[6-[[5-chloro-2-[[(6R,7S)-7-[3-(2,6-dioxo-3-piperidyl)-1-methyl-indazol-6-yl]oxy-6-methyl-2-azaspiro[3.5]nonan-2-yl]pyrimidin-4-yl]amino]-1-isopropyl-2-oxo-1,8-naphthyridin-3-yl]oxy]-N-methyl-acetamide [rel-2-((6-((5-chloro-2-((6R,7S)-7-((3-(2,6-dioxopiperidin-3-yl)-1-methyl-1H-indazol-6-yl)oxy)-6-methyl-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide] (18.46 mg, 22.92 μmol) as a pink solid.

LC-MS (ESI): m/z=797.4 [M+H]⁺

¹H NMR (400 MHz, DMSO-d₆) δ=10.86 (s, 1H), 9.02 (s, 1H), 8.81-8.72 (m, 1H), 8.45 (s, 1H), 8.06 (s, 1H), 8.01-7.93 (m, 1H), 7.56 (d, J=9.2 Hz, 1H), 7.15 (s, 1H), 7.07 (s, 1H), 6.81-6.71 (m, 1H), 6.04-5.91 (m, 1H), 4.59 (s, 2H), 4.52 (s, 1H), 4.29 (dd, J=5.2, 9.6 Hz, 1H), 3.91 (s, 3H), 3.77 (s, 2H), 3.68 (s, 2H), 2.69-2.65 (m, 5H), 2.33 (s, 1H), 2.15 (dd, J=5.6, 12.8 Hz, 1H), 2.02 (d, J=12.0 Hz, 1H), 1.91-1.83 (m, 1H), 1.76-1.63 (m, 4H), 1.57 (d, J=6.8 Hz, 6H), 1.23 (s, 1H), 0.99 (d, J=6.0 Hz, 3H)

### Example 73: 2-[[6-[[5-chloro-2-[(6S,7R)-7-[3-(2,6-dioxo-3-piperidyl)-1-methyl-indazol-6-yl]oxy-6-methyl-2-azaspiro[3.5]nonan-2-yl]pyrimidin-4-yl]amino]-1-isopropyl-2-oxo-1,8-naphthyridin-3-yl]oxy]-N-methyl-acetamide [rel-2-((6-((5-chloro-2-((6S,7R)-7-((3-(2,6-dioxopiperidin-3-yl)-1-methyl-1H-indazol-6-yl)oxy)-6-methyl-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide] (Compound 185b)

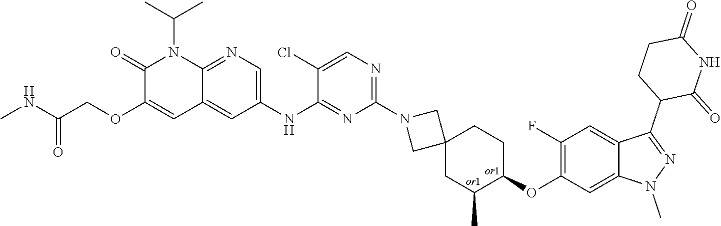 [SMILES: CNC(=O)COc1cc2cc(Nc3nc(N4CC5(CC[C@@H](Oc6cc7c(cc6F)c(C6CCC(=O)NC6=O)nn7C)[C@@H](C)C5)C4)ncc3Cl)cnc2n(C(C)C)c1=O]

### Preparation of tert-butyl (6S,7R)-7-[3-(2,6-dibenzyloxy-3-pyridyl)-1-methyl-indazol-6-yl]oxy-6-methyl-2-azaspiro[3.5]nonane-2-carboxylate

A mixture of tert-butyl (6S,7S)-7-hydroxy-6-methyl-2-azaspiro[3.5]nonane-2-carboxylate (0.5 g, 1.96 mmol), 3-(2,6-dibenzyloxy-3-pyridyl)-1-methyl-indazol-6-ol (1.03 g, 2.35 mmol), and 2-(tributylphosphanylidene) acetonitrile (519.85 mg, 2.15 mmol) in toluene (6 mL) was degassed and purged with N₂ three times. The mixture was stirred at 120° C. for 16 h under N₂ atmosphere. The reaction mixture was concentrated under reduced pressure to remove toluene. The residue was purified by prep-HPLC to afford tert-butyl (6S,7R)-7-[3-(2,6-dibenzyloxy-3-pyridyl)-1-methyl-indazol-6-yl]oxy-6-methyl-2-azaspiro[3.5]nonane-2-carboxylate (0.52 g, 770.57 μmol) as a brown oil.

LC-MS (ESI): m/z=675.4 [M+H]⁺

### Preparation of tert-butyl (6S,7R)-7-[3-(2,6-dioxo-3-piperidyl)-1-methyl-indazol-6-yl]oxy-6-methyl-2-azaspiro[3.5]nonane-2-carboxylate

A mixture of tert-butyl (6S,7R)-7-[3-(2,6-dibenzyloxy-3-pyridyl)-1-methyl-indazol-6-yl]oxy-6-methyl-2-azaspiro[3.5]nonane-2-carboxylate (500 mg, 740.93 μmol), Pd/C (236.55 mg, 222.28 μmol, 10% purity), AcOH (4.45 mg, 74.09 μmol, 4.24 μL) in CF₃CH₂OH (6 mL) was degassed and purged with H₂ three times. The mixture was stirred at 25° C. for 16 h under H₂ atmosphere. The reaction mixture was filtered, and the filtrate was concentrated to give a residue. The residue was purified by flash silica gel chromatography (0~65% ethyl acetate/petroleum ether) to afford tert-butyl (6S,7R)-7-[3-(2,6-dioxo-3-piperidyl)-1-methyl-indazol-6-yl]oxy-6-methyl-2-azaspiro[3.5]nonane-2-carboxylate (200 mg, 382.60 μmol) as a colorless oil.

LC-MS (ESI): m/z=497.5 [M+H]⁺

### Preparation of 3-[1-methyl-6-[[(6S,7R)-6-methyl-2-azaspiro[3.5]nonan-7-yl]oxy] indazol-3-yl]piperidine-2,6-dione

A solution of tert-butyl (6S,7R)-7-[3-(2,6-dioxo-3-piperidyl)-1-methyl-indazol-6-yl]oxy-6-methyl-2-azaspiro[3.5]nonane-2-carboxylate (200 mg, 402.74 μmol) in DCM (1.5 mL) and TFA (0.5 mL) was stirred at 25° C. for 0.5 h. The reaction mixture was concentrated under reduced pressure to give 3-[1-methyl-6-[(6S,7R)-6-methyl-2-azaspiro[3.5]nonan-7-yl]oxy Jindazol-3-yl]piperidine-2,6-dione (155 mg, 387.03 μmol) as a brown solid.

LC-MS (ESI): m/z=397.3 [M+H]⁺

¹H NMR (400 MHz, DMSO-d₆) δ=10.87 (s, 1H), 8.74-8.52 (m, 2H), 7.55 (d, J=8.8 Hz, 1H), 7.07 (d, J=1.6 Hz, 1H), 6.75 (dd, J=2.0, 8.9 Hz, 1H), 4.48 (s, 1H), 4.29 (dd, J=5.2, 9.6 Hz, 1H), 4.03 (q, J=7.2 Hz, 1H), 3.84-3.70 (m, 2H), 3.70-3.59 (m, 2H), 2.36-2.30 (m, 1H), 2.20-2.09 (m, 1H), 2.03-1.95 (m, 3H), 1.79 (t, J=10.0 Hz, 2H), 1.71-1.58 (m, 2H), 1.52-1.38 (m, 1H), 1.17 (t, J=7.2 Hz, 2H), 0.98 (d, J=6.4 Hz, 3H).

### Preparation of 2-[[6-[[5-chloro-2-[(6S,7R)-7-[3-(2,6-dioxo-3-piperidyl)-1-methyl-indazol-6-yl]oxy-6-methyl-2-azaspiro[3.5]nonan-2-yl]pyrimidin-4-yl]amino]-1-isopropyl-2-oxo-1,8-naphthyridin-3-yl]oxy]-N-methyl-acetamide [rel-2-((6-((5-chloro-2-((6S,7R)-7-((3-(2,6-dioxopiperidin-3-yl)-1-methyl-1H-indazol-6-yl)oxy)-6-methyl-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide]

To a solution of 2-[[6-[(2,5-dichloropyrimidin-4-yl)amino]-1-isopropyl-2-oxo-1,8-naphthyridin-3-yl]oxy]-N-methylacetamide (50 mg, 114.34 u mol) and 3-[1-methyl-6-[[(6S,7R)-6-methyl-2-azaspiro[3.5]nonan-7-yl]oxy] indazol-3-yl]piperidine-2,6-dione (54.40 mg, 137.21 μmol) in DMSO (1 mL) was added Et₃N (46.28 mg, 457.37 μmol, 63.66 μL). The mixture was stirred at 100° C. for 2 h. The reaction mixture was filtered, and the filtrate was concentrated to give a residue. The residue was purified by prep-HPLC to afford 2-[[6-[[5-chloro-2-[(6S,7R)-7-[3-(2,6-dioxo-3-piperidyl)-1-methyl-indazol-6-yl]oxy-6-methyl-2-azaspiro[3.5]nonan-2-yl]pyrimidin-4-yl]amino]-1-isopropyl-2-oxo-1,8-naphthyridin-3-yl]oxy]-N-methyl-acetamide [rel-2-((6-((5-chloro-2-((6S,7R)-7-((3-(2,6-dioxopiperidin-3-yl)-1-methyl-1H-indazol-6-yl)oxy)-6-methyl-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide] (25.75 mg, 30.81 μmol) was obtained as a yellow solid.

LC-MS (ESI): m/z=797.3 [M+H]⁺.

¹H NMR (400 MHz, DMSO-d₆) δ=10.98-10.69 (m, 1H), 9.11-8.94 (m, 1H), 8.77 (s, 1H), 8.50-8.42 (m, 2H), 8.07-7.94 (m, 1H), 7.56 (d, J=8.8 Hz, 1H), 7.26-7.11 (m, 1H), 7.10-6.95 (m, 1H), 6.84-6.72 (m, 1H), 6.13-5.68 (m, 1H), 4.59 (s, 2H), 4.52 (s, 1H), 4.29 (dd, J=5.2, 9.2 Hz, 1H), 3.91 (s, 3H), 3.77 (s, 2H), 3.71-3.61 (m, 2H), 2.73-2.59 (m, 5H), 2.35-2.28 (m, 2H), 2.21-2.08 (m, 1H), 2.07-1.94 (m, 1H), 1.92-1.80 (m, 1H), 1.79-1.63 (m, 3H), 1.57 (d, J=6.8 Hz, 6H), 1.18 (s, 1H), 0.99 (d, J=6.0 Hz, 3H)

### Example 74: 2-[[6-[[5-chloro-2-[(6S,7S)-7-[3-(2,6-dioxo-3-piperidyl)-1-methyl-indazol-6-yl]oxy-6-methyl-2-azaspiro[3.5]nonan-2-yl]pyrimidin-4-yl]amino]-1-isopropyl-2-oxo-1,8-naphthyridin-3-yl|oxy]-N-methyl-acetamide [rel-2-((6-((5-chloro-2-((6S,7S)-7-((3-(2,6-dioxopiperidin-3-yl)-1-methyl-1H-indazol-6-yl)oxy)-6-methyl-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide] (Compound 185c)

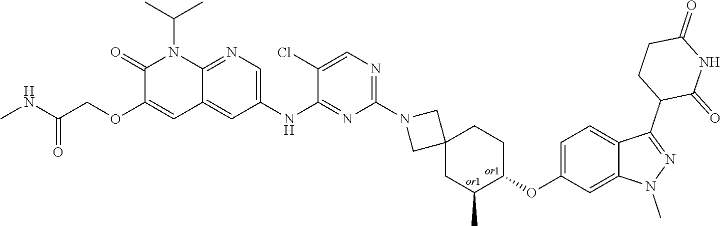 [SMILES: CNC(=O)COc1cc2cc(Nc3nc(N4CC5(CC[C@H](Oc6ccc7c(C8CCC(=O)NC8=O)nn(C)c7c6)[C@@H](C)C5)C4)ncc3Cl)cnc2n(C(C)C)c1=O]

### Preparation of tert-butyl 6-methyl-7-oxo-2-azaspiro[3.5]nonane-2-carboxylate

To a solution of LiHMDS (55.935 g, 334.28 mL, 1 M, 334.28 mmol) in THF (200 mL) under N₂ was added a solution of tert-butyl 7-oxo-2-azaspiro[3.5]nonane-2-carboxylate (4.0 g, 167.14 mmol) in THF (200 mL) dropwise. The resulting mixture was stirred at −78° C. for 1 h followed by the addition of iodomethane (47.448 g, 20.8 mL, 334.28 mmol). The mixture was stirred at 25° C. for 12 h. The reaction mixture was quenched by addition of saturated NH₄Cl solution (200 mL) and extracted with ethyl acetate (70 mL×3). The combined organic layers were washed with brine (70 mL×2), dried over Na₂SO₄, filtered, and concentrated under reduced pressure to give a residue. The residue was purified by column chromatography (petroleum ether/ethyl acetate=10:1) to give tert-butyl 6-methyl-7-oxo-2-azaspiro[3.5]nonane-2-carboxylate (27 g, 0.11 mol) as a white solid.

¹H NMR (400 MHz, DMSO-d₆) δ=3.83 (br s, 2H), 3.53 (br s, 2H), 2.48-2.39 (m, 1H), 2.21-2.06 (m, 3H), 1.77 (dt, J=4.4, 13.6 Hz, 1H), 1.52 (t, J=12.8 Hz, 1H), 1.39 (s, 9H), 0.88 (d, J=6.4 Hz, 3H)

### Preparation of tert-butyl 7-hydroxy-6-methyl-2-azaspiro[3.5]nonane-2-carboxylate

To a solution of tert-butyl 6-methyl-7-oxo-2-azaspiro[3.5]nonane-2-carboxylate (26 g, 102.63 mmol) in MeOH (300 mL) was added NaBH₄ (11.65 g, 307.89 mmol) at 0° C. The mixture was stirred at 0° C. for 1.5 h. The reaction mixture diluted with NH₄Cl (500 mL) and extracted with ethyl acetate (90 mL×3). The combined organic layers were washed with brine (100 mL×2), dried over Na₂SO₄, filtered, and concentrated under reduced pressure to give a residue. The residue was purified by column chromatography (petroleum ether/ethyl acetate=10/1 to 6/1) to afford tert-butyl 7-hydroxy-6-methyl-2-azaspiro[3.5]nonane-2-carboxylate (6.8 g, 21.54 mmol) as a white solid.

LC-MS (ESI): m/z=200.2 [M+H]⁺

¹H NMR: (400 MHz, MeOD-d4) δ=3.69-3.48 (m, 4H), 3.09-2.94 (m, 1H), 1.93-1.80 (m, 3H), 1.57-1.47 (m, 1H), 1.43 (s, 9H), 1.33-1.18 (m, 3H), 1.01 (br d, J=6.0 Hz, 3H)

### Preparation of tert-butyl (6S,7R)-7-hydroxy-6-methyl-2-azaspiro[3.5]nonane-2-carboxylate, tert-butyl (6R,7S)-7-hydroxy-6-methyl-2-azaspiro[3.5]nonane-2-carboxylate, tert-butyl (6S,7S)-7-hydroxy-6-methyl-2-azaspiro[3.5]nonane-2-carboxylate, and tert-butyl (6R,7R)-7-hydroxy-6-methyl-2-azaspiro[3.5]nonane-2-carboxylate

Stereoisomers of tert-butyl 7-hydroxy-6-methyl-2-azaspiro[3.5]nonane-2-carboxylate (2.6 g, 10.18 mmol) were separated by SFC to afford tert-butyl (6S,7R)-7-hydroxy-6-methyl-2-azaspiro[3.5]nonane-2-carboxylate (622 mg, 2.31 mmol), tert-butyl (6R,7S)-7-hydroxy-6-methyl-2-azaspiro[3.5]nonane-2-carboxylate (1.17 g, 4.20 mmol), tert-butyl (6S,7S)-7-hydroxy-6-methyl-2-azaspiro[3.5]nonane-2-carboxylate (3.3 g, 12.76 mmol), and tert-butyl (6R,7R)-7-hydroxy-6-methyl-2-azaspiro[3.5]nonane-2-carboxylate (3.3 g, 12.80 mmol).

LC-MS (ESI): m/z=200.2 [M+H]⁺

LC-MS (ESI): m/z=200.2 [M+H]⁺

LC-MS (ESI): m/z=200.2 [M+H]⁺

LC-MS (ESI): m/z=200.2 [M+H]⁺

¹H NMR: (400 MHz, DMSO-d₆) δ=4.21 (d, J=4.0 Hz, 1H), 3.53-3.39 (m, 4H), 1.75-1.55 (m, 2H), 1.53-1.38 (m, 5H), 1.36 (s, 9H), 0.85 (br d, J=6.0 Hz, 3H).

¹H NMR: (400 MHz, DMSO-d₆) δ=4.21 (d, J=4.0 Hz, 1H), 3.52-3.39 (m, 4H), 1.72-1.55 (m, 2H), 1.52-1.37 (m, 5H), 1.36 (s, 9H), 0.88-0.81 (m, 3H).

¹H NMR: (400 MHz, DMSO-d₆) δ=4.43 (d, J=5.2 Hz, 1H), 3.51 (br s, 2H), 3.42 (br s, 2H), 2.85 (tt, J=4.8, 9.6 Hz, 1H), 1.83-1.63 (m, 3H), 1.36 (s, 9H), 1.22-1.02 (m, 3H), 0.91 (d, J=6.0 Hz, 3H).

¹H NMR: (400 MHz, DMSO-d₆) δ=4.43 (d, J=5.2 Hz, 1H), 3.51 (br s, 2H), 3.42 (br s, 2H), 2.90-2.81 (m, 1H), 1.79-1.65 (m, 3H), 1.36 (s, 9H), 1.21-1.02 (m, 3H), 0.91 (d, J=6.0 Hz, 3H).

### Preparation of tert-butyl (6S,7S)-7-[3-(2,6-dibenzyloxy-3-pyridyl)-1-methyl-indazol-6-yl]oxy-6-methyl-2-azaspiro[3.5]nonane-2-carboxylate

A mixture of 6-bromo-3-(2,6-dibenzyloxy-3-pyridyl)-1-methyl-indazole (1 g, 2.00 mmol, 1 eq), tert-butyl (6S,7S)-7-hydroxy-6-methyl-2-azaspiro[3.5]nonane-2-carboxylate (1.02 g, 4.00 mmol), Ir[dF(CF3)ppy]₂(dtbpy)(PF₆) (22.42 mg, 19.98 μmol), NiCl₂.dtbbpy (39.77 mg, 99.92 μmol), quinuclidine (22.22 mg, 199.85 μmol), and TMP (564.58 mg, 4.00 mmol, 678.58 μL) in MeCN (33 mL) was purged with nitrogen three times. The reaction vial was then sealed with parafilm, placed 2 cm away from one blue LED, and irradiated at 25° C. for 14 h. The reaction mixture was concentrated under reduced pressure to give a residue. The residue was purified by flash silica gel chromatography (30% ethyl acetate/petroleum ether) to afford tert-butyl (6S,7S)-7-[3-(2,6-dibenzyloxy-3-pyridyl)-1-methyl-indazol-6-yl]oxy-6-methyl-2-azaspiro[3.5]nonane-2-carboxylate (1.4 g, 1.66 mmol) as a white solid.

LC-MS (ESI): m/z=675.5 [M+H]⁺.

¹H NMR (400 MHz, DMSO-d₆) δ=7.90-7.85 (m, 1H), 7.54-7.49 (m, 1H), 7.49-7.44 (m, 2H), 7.42-7.31 (m, 6H), 7.30-7.28 (m, 1H), 6.64-6.53 (m, 2H), 5.43 (d, J=11.2 Hz, 4H), 3.99 (s, 3H), 3.51 (s, 5H), 2.08-1.85 (m, 1H), 1.94-1.84 (m, 2H), 1.78-1.72 (m, 4H), 1.38 (s, 9H), 1.00-0.95 (m, 3H).

### Preparation of tert-butyl (6S,7S)-7-[3-(2,6-dioxo-3-piperidyl)-1-methyl-indazol-6-yl]oxy-6-methyl-2-azaspiro[3.5]nonane-2-carboxylate

To a solution of tert-butyl (6S,7S)-7-[3-(2,6-dibenzyloxy-3-pyridyl)-1-methyl-indazol-6-yl]oxy-6-methyl-2-azaspiro[3.5]nonane-2-carboxylate (1.30 g, 1.93 mmol) in CF₃CH₂OH (13 mL) was added Pd/C (615.03 mg, 577.93 μmol, 10% purity) and AcOH (11.57 mg, 192.64 μmol, 11.03 μL) under N₂ atmosphere. The suspension was degassed and purged with H₂ three times. The mixture was stirred under H₂ (15 psi) at 25° C. for 16 hr. The reaction mixture was filtered and concentrated under reduced pressure to give a residue. The residue was purified by flash silica gel chromatography (60-100% ethyl acetate/petroleum ether) to afford tert-butyl (6S,7S)-7-[3-(2,6-dioxo-3-piperidyl)-1-methyl-indazol-6-yl]oxy-6-methyl-2-azaspiro[3.5]nonane-2-carboxylate (400 mg, 792.92 μmol) as a white solid.

LC-MS (ESI): m/z=497.4 [M+H]⁺

¹H NMR (400 MHz, DMSO-d₆) δ=10.85 (s, 1H), 7.54 (d, J=9.2 Hz, 1H), 7.06 (s, 1H), 6.72 (d, J=8.8 Hz, 1H), 4.35-4.27 (m, 1H), 4.00 (s, 1H), 3.92 (s, 3H), 3.61 (s, 2H), 3.51 (s, 2H), 2.70-2.57 (m, 2H), 2.34-2.28 (m, 1H), 2.19-2.03 (m, 2H), 1.95-1.82 (m, 2H), 1.71-1.56 (m, 2H), 1.38 (s, 9H), 1.32-1.23 (m, 2H), 0.98 (d, J=6.0 Hz, 3H).

### Preparation of 3-[1-methyl-6-[[(6S,7S)-6-methyl-2-azaspiro[3.5]nonan-7-yl]oxy] indazol-3-yl]piperidine-2,6-dione

To a solution of tert-butyl (6S,7S)-7-[3-(2,6-dioxo-3-piperidyl)-1-methyl-indazol-6-yl]oxy-6-methyl-2-azaspiro[3.5]nonane-2-carboxylate (400 mg, 805.48 μmol) in DCM (3 mL) was added TFA (1 mL). The mixture was stirred at 25° C. for 0.5 h. The reaction mixture was concentrated under reduced pressure to give 3-[1-methyl-6-[[(6S,7S)-6-methyl-2-azaspiro[3.5]nonan-7-yl]oxy] indazol-3-yl]piperidine-2,6-dione (590 mg, crude, TFA) was obtained as a colorless gum

LC-MS (ESI): m/z=397.3 [M+H]⁺

### Preparation of 2-[[6-[[5-chloro-2-[(6S,7S)-7-[3-(2,6-dioxo-3-piperidyl)-1-methyl-indazol-6-yl]oxy-6-methyl-2-azaspiro[3.5]nonan-2-yl]pyrimidin-4-yl]amino]-1-isopropyl-2-oxo-1,8-naphthyridin-3-yl]oxy]-N-methyl-acetamide [rel-2-((6-((5-chloro-2-((6S,7S)-7-((3-(2,6-dioxopiperidin-3-yl)-1-methyl-1H-indazol-6-yl)oxy)-6-methyl-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide]

To a solution of 2-[[6-[(2,5-dichloropyrimidin-4-yl)amino]-1-isopropyl-2-oxo-1,8-naphthyridin-3-yl]oxy]-N-methyl-acetamide (100 mg, 228.69 μmol) in DMSO (1 mL) was added TEA (92.56 mg, 914.75 μmol, 127.32 μL) and 3-[1-methyl-6-[[(6S,7S)-6-methyl-2-azaspiro[3.5]nonan-7-yl]oxy]indazol-3-yl]piperidine-2,6-dione (136.01 mg, 343.03 μmol). The mixture was stirred at 100° C. for 1 h. The reaction mixture was filtered to give a residue. The residue was purified by prep-HPLC to afford 2-[[6-[[5-chloro-2-[(6S,7S)-7-[3-(2,6-dioxo-3-piperidyl)-1-methyl-indazol-6-yl]oxy-6-methyl-2-azaspiro[3.5]nonan-2-yl]pyrimidin-4-yl]amino]-1-isopropyl-2-oxo-1,8-naphthyridin-3-yl]oxy]-N-methyl-acetamide [rel-2-((6-((5-chloro-2-((6S,7S)-7-((3-(2,6-dioxopiperidin-3-yl)-1-methyl-1H-indazol-6-yl)oxy)-6-methyl-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide] (76 mg, 95.32 μmol) as a white solid.

LC-MS (ESI): m/z=797.5 [M+H]⁺.

¹H NMR (400 MHz, DMSO-d₆) δ=10.86 (s, 1H), 9.03 (s, 1H), 8.78 (s, 1H), 8.41 (s, 1H), 8.16-8.01 (m, 1H), 8.00-7.86 (m, 1H), 7.66-7.44 (m, 1H), 7.17 (s, 1H), 7.11-7.01 (m, 1H), 6.81-6.66 (m, 1H), 6.06-5.87 (m, 1H), 4.65-4.50 (m, 2H), 4.34-4.23 (m, 1H), 4.09-3.99 (m, 1H), 3.92 (s, 3H), 3.84-3.73 (m, 2H), 3.72-3.63 (m, 2H), 2.68 (s, 3H), 2.54 (s, 2H), 2.31-2.25 (m, 1H), 2.20-2.05 (m, 2H), 2.03-1.88 (m, 2H), 1.79-1.65 (m, 2H), 1.65-1.48 (m, 6H), 1.47-1.27 (m, 2H), 1.00-0.99 (m, 3H).

### Example 75: 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-(2-hydroxyethyl)-2-oxo-1,2-dihydro-1,8-naphthyridin-3-yl)oxy)-N-methylacetamide (Compound 186)

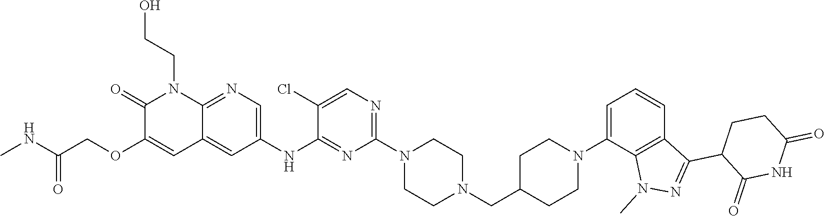 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)cnc2n(CCO)c1=O]

### Preparation of 1-(2-methoxyethyl)-5-nitro-1H-pyrrolo[2,3-b]pyridine

To a stirred solution of 5-nitro-1H-pyrrolo[2,3-b]pyridine (20.0 g, 0.13 mol) and potassium carbonate (44.16 g, 0.32 mol) in DMF (400 mL) was added 1-bromo-2-methoxyethane (44.85 g, 0.32 mol) drop-wise. The mixture was stirred at room temperature for 16 h. Ice-cold water (1000 mL) was added slowly to the reaction mixture, and the resulting precipitate was filtered and dried under vacuum to afford 1-(2-methoxyethyl)-5-nitro-1H-pyrrolo[2,3-b]pyridine (30 g) as an off-white solid which was used as such for the next step.

LC-MS (ESI): m/z=222.03 [M+H]⁺

### Preparation of 1-(2-methoxyethyl)-5-nitro-1H-pyrrolo[2,3-b]pyridine-2,3-dione

To a stirred solution of 1-(2-methoxyethyl)-5-nitro-1H-pyrrolo[2,3-b]pyridine (20.0 g, 0.09 mol) in acetone (170 mL), acetic acid (640 mL), and H₂O (200 mL) at 0° C. was added CrO₃ (117.16 g, 1.17 mol) slowly. The mixture was stirred at room temperature for 0.5 h. The reaction mixture was diluted with water (500 mL) and extracted with DCM (500 mL×4). The separated organic layers were combined, washed with brine (200 mL) and dried over Na₂SO₄, filtered, and concentrated to afford 1-(2-methoxyethyl)-5-nitro-1H-pyrrolo[2,3-b]pyridine-2,3-dione (20 g) as a pale-yellow solid which was used as such for next step without further purification.

LC-MS (ESI): m/z=270.20 [M+H₂O]⁺

### Preparation of 3-hydroxy-1-(2-methoxyethyl)-6-nitro-1,8-naphthyridin-2 (1H)-one

To a stirred solution of 1-(2-methoxyethyl)-5-nitro-1H-pyrrolo[2,3-b]pyridine-2,3-dione (20.0 g, 0.08 mol) in EtOH (400 mL) was added triethyl amine (23 mL, 0.17 mol) and (trimethylsilyl diazomethane (2.0 M in diethyl ether) (17 mL, 0.17 mol) at 0° C. The mixture was stirred at room temperature for 4 h. The reaction mixture was diluted with water (300 mL) and extracted with ethyl acetate (300 mL×3). The combined organic layer was washed with brine, dried over Na₂SO₄, and concentrated to afford crude 3-hydroxy-1-(2-methoxyethyl)-6-nitro-1,8-naphthyridin-2 (1H)-one (5.0 g) as a brown solid which was used as such for next step without further purification.

LC-MS (ESI): m/z=266.03 [M+H]

### Preparation of 3-hydroxy-1-(2-hydroxyethyl)-6-nitro-1,8-naphthyridin-2 (1H)-one

To a stirred solution of 3-hydroxy-1-(2-methoxyethyl)-6-nitro-1,8-naphthyridin-2 (1H)-one (5.0 g, 0.018 mol) in DCM (100 mL) was added BBr₃ (1M in DCM, 2.8 mL, 0.028 mol) at 0° C. The mixture was stirred at room temperature for 2 h. The reaction was quenched with cold water (500 mL) and extracted with 10% MeOH in DCM (300 mL×3). The combined organic layer was washed with brine, dried over Na₂SO₄, and concentrated to afford a crude product which was purified by column chromatography (0-100% ethyl acetate in petroleum ether) to afford 3-hydroxy-1-(2-hydroxyethyl)-6-nitro-1,8-naphthyridin-2 (1H)-one (2.0 g) as a solid.

LC-MS (ESI): m/z=251.99 [M+H]⁺

### Preparation of 1-(2-((tert-butyldimethylsilyl)oxy) ethyl)-3-hydroxy-6-nitro-1,8-naphthyridin-2 (1H)-one

To a stirred solution of 3-hydroxy-1-(2-hydroxyethyl)-6-nitro-1,8-naphthyridin-2 (1H)-one (2.0 g, 0.0079 mol) in THF (40 mL) were added imidazole (0.644 g, 0.0095 mol) and TBDMS-Cl (1.43 g, 0.0095 mol). The mixture was stirred at room temperature for 2 h. The reaction was quenched with cold water (250 mL) and extracted with ethyl acetate (100 mL×3). The combined organic layer was washed with brine, dried over Na₂SO₄, and concentrated to afford a crude product which was purified by column chromatography (SiO₂, 100-200 mesh; 0-100% ethyl acetate in petroleum ether) to afford 1-(2-((tert-butyldimethylsilyl)oxy) ethyl)-3-hydroxy-6-nitro-1,8-naphthyridin-2 (1H)-one (0.25 g) as an off white solid.

LC-MS (ESI): m/z=366.15 [M+H]⁺

### Preparation of 2-((1-(2-((tert-butyldimethylsilyl)oxy) ethyl)-6-nitro-2-oxo-1,2-dihydro-1,8-naphthyridin-3-yl)oxy)-N-methylacetamide

To a stirred solution of 1-(2-((tert-butyldimethylsilyl)oxy) ethyl)-3-hydroxy-6-nitro-1,8-naphthyridin-2 (1H)-one (0.25 g, 0.68 mol) in DMF (5 mL) was added cesium carbonate (0.332 g, 1.02 mol) and 2-bromo-N-methylacetamide (0.123 g, 0.816 mol) at 0° C. The mixture was stirred at room temperature for 1 h. Ice water (100 mL) was added to the reaction mixture and the resultant precipitate was filtered and dried under vacuum to afford 2-((1-(2-((tert-butyldimethylsilyl)oxy) ethyl)-6-nitro-2-oxo-1,2-dihydro-1,8-naphthyridin-3-yl)oxy)-N-methylacetamide (0.2 g) as an off-white solid.

LC-MS (ESI): m/z=437.18 [M+H]⁺

### Preparation of 2-((6-amino-1-(2-((tert-butyldimethylsilyl)oxy) ethyl)-2-oxo-1,2-dihydro-1,8-naphthyridin-3-yl)oxy)-N-methylacetamide

To a stirred suspension of 2-((1-(2-((tert-butyldimethylsilyl)oxy) ethyl)-6-nitro-2-oxo-1,2-dihydro-1,8-naphthyridin-3-yl)oxy)-N-methylacetamide (0.2 g, 0.45 mmol) in MeOH (4 mL) was added iron (0.256 g, 4.58 mol) and ammonium chloride (0.244 g, 4.58 mmol) at room temperature. The mixture was heated at 80° C. for 4 h. The reaction was quenched with cold water (100 mL) and extracted with ethyl acetate (50 mL×3). The combined organic layer was washed with brine, dried over Na₂SO₄, and concentrated to afford 2-((6-amino-1-(2-((tert-butyldimethylsilyl)oxy) ethyl)-2-oxo-1,2-dihydro-1,8-naphthyridin-3-yl)oxy)-N-methylacetamide (0.15 g) as a solid.

LC-MS (ESI): m/z=407.46 [M+H]

### Preparation of 2-((1-(2-((tert-butyldimethylsilyl)oxy) ethyl)-6-((2,5-dichloropyrimidin-4-yl)amino)-2-oxo-1,2-dihydro-1,8-naphthyridin-3-yl)oxy)-N-methylacetamide

To a stirred solution of 2-((6-amino-1-(2-((tert-butyldimethylsilyl)oxy) ethyl)-2-oxo-1,2-dihydro-1,8-naphthyridin-3-yl)oxy)-N-methylacetamide (0.15 g, 0.36 mmol) in DMSO (3 mL) were added N,N-diisopropylethylamine (0.5 mL, 2.16 mol) and 2,4,5-trichloropyrimidine (0.806 g, 0.468 mmol) at room temperature. The mixture was stirred at 110° C. for 16 h. Ice cold water (100 mL) was added to the reaction mixture, and the resulting precipitate was filtered and dried under vacuum to afford 2-((1-(2-((tert-butyldimethylsilyl)oxy) ethyl)-6-((2,5-dichloropyrimidin-4-yl)amino)-2-oxo-1,2-dihydro-1,8-naphthyridin-3-yl)oxy)-N-methylacetamide (0.15 g) as a white solid.

LC-MS (ESI): m/z=553.47 [M+H]⁺

### Preparation of 2-((6-((2,5-dichloropyrimidin-4-yl)amino)-1-(2-hydroxyethyl)-2-oxo-1,2-dihydro-1,8-naphthyridin-3-yl)oxy)-N-methylacetamide

To a stirred solution of 2-((1-(2-((tert-butyldimethylsilyl)oxy) ethyl)-6-((2,5-dichloropyrimidin-4-yl)amino)-2-oxo-1,2-dihydro-1,8-naphthyridin-3-yl)oxy)-N-methylacetamide (0.150 g, 0.27 mmol) in THF (3 mL) was added TBAF (1M in THF) (0.4 mL, 0.40 mmol) at 0° C. The mixture was stirred at room temperature for 4 h. Ice cold water (50 mL) was added, and the mixture was extracted with ethyl acetate (20 mL×3). The combined organic layer was washed with brine, dried over Na₂SO₄ and concentrated to afford 2-((6-((2,5-dichloropyrimidin-4-yl)amino)-1-(2-hydroxyethyl)-2-oxo-1,2-dihydro-1,8-naphthyridin-3-yl) oxy)-N-methylacetamide 0.06 g) as an off-white solid.

LC-MS (ESI): m/z=437.01 [M−H]⁻

### Preparation of 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-(2-hydroxyethyl)-2-oxo-1,2-dihydro-1,8-naphthyridin-3-yl)oxy)-N-methylacetamide

To a stirred suspension of 3-(1-methyl-7-(4-(piperazin-1-ylmethyl)piperidin-1-yl)-1H-indazol-3-yl)piperidine-2,6-dione (0.08 g, 0.18 mmol) in DMSO (1.5 mL) were added N,N-diisopropylethylamine (0.19 mL, 1.08 mol) and 2-((6-((2,5-dichloropyrimidin-4-yl)amino)-1-(2-hydroxyethyl)-2-oxo-1,2-dihydro-1,8-naphthyridin-3-yl)oxy)-N-methylacetamide (0.048 g, 0.12 mmol) at room temperature. The reaction mass was heated at 110° C. and stirred for 16 h. The reaction was concentrated under reduced pressure to obtain a crude product which was purified by prep-HPLC to afford 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-(2-hydroxyethyl)-2-oxo-1,2-dihydro-1,8-naphthyridin-3-yl)oxy)-N-methylacetamide (0.006 g) as an off-white solid.

¹H NMR: (400 MHz, DMSO-d₆): δ 10.88 (s, 1H), 9.39 (br s, 1H), 9.27 (s, 1H), 8.67 (d, J=2.4 Hz, 1H), 8.28 (d, J=2.0 Hz, 1H), 8.19 (s, 1H), 7.96 (d, J=4.4 Hz, 1H), 7.41-7.36 (m, 1H), 7.22-6.87 (m, 4H), 4.89 (br s, 1H), 4.61 (s, 2H), 4.55 (t, J=6.8 Hz, 2H), 4.44 (br s, 1H), 4.40-4.30 (m, 1H), 4.25 (s, 3H), 3.65-3.57 (m, 4H), 3.20-3.33 (m, 3H), 2.75-260 (m, 3H), 2.32 (s, 1H), 2.18 (s, 1H), 1.90 (d, J=7.2 Hz, 2H), 1.50 (s, 2H), 1.35 (s, 3H), 1.25-1.20 (m, 6H).

LC-MS (ESI): m/z=827.47 [M+H]⁺

### Example 76: 2-((6-((5-chloro-2-(4-((1S)-1-(1-(3-(2,6-dioxopiperidin-3-yl)-1-methyl-1H-indazol-7-yl)piperidin-4-yl)-1-hydroxyethyl)piperidin-1-yl)pyrimidin-4-yl)amino)-1-isopropyl-2-oxo-1,2-dihydro-1,8-naphthyridin-3-yl)oxy)-N-methylacetamide [rel-2-((6-((5-chloro-2-(4-((1S)-1-(1-(3-(2,6-dioxopiperidin-3-yl)-1-methyl-1H-indazol-7-yl)piperidin-4-yl)-1-hydroxyethyl)piperidin-1-yl)pyrimidin-4-yl)amino)-1-isopropyl-2-oxo-1,2-dihydro-1,8-naphthyridin-3-yl)oxy)-N-methylacetamide] (Compound 194a)

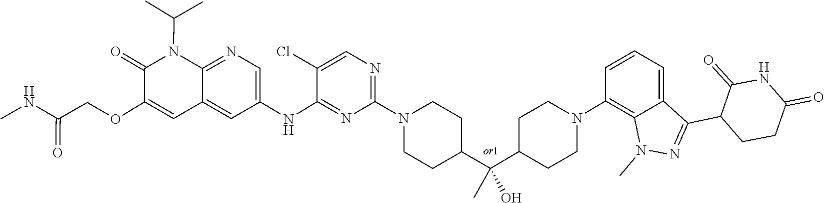 [SMILES: CNC(=O)COc1cc2cc(Nc3nc(N4CCC([C@@](C)(O)C5CCN(c6cccc7c(C8CCC(=O)NC8=O)nn(C)c67)CC5)CC4)ncc3Cl)cnc2n(C(C)C)c1=O]

### Preparation of benzyl 4-((pyridin-2-ylthio) carbonyl)piperidine-1-carboxylate

To a stirred solution of 1-((benzyloxy) carbonyl)piperidine-4-carboxylic acid (10.0 g, 37.98 mmol) and 1,2-di (pyridin-2-yl)disulfane (9.2 g, 41.77 mmol) in MeCN (200 mL) was added triphenylphosphine (10.90 g, 41.77 mmol). The mixture was stirred at room temperature for 16 h. The reaction mixture was concentrated under reduced pressure to get the crude product which was purified by flash column chromatography (20% ethyl acetate in petroleum ether) to afford benzyl 4-((pyridin-2-ylthio) carbonyl)piperidine-1-carboxylate as a yellow liquid (8.0 g).

LC-MS (ESI): m/z=357.40 [M+H]⁺

### Preparation of benzyl 4-(1-(tert-butoxycarbonyl)piperidine-4-carbonyl)piperidine-1-carboxylate

To a stirred solution of benzyl 4-((pyridin-2-ylthio) carbonyl)piperidine-1-carboxylate (8.0 g, 22.44 mmol), 1-(tert-butyl) 4-(1,3-dioxoisoindolin-2-yl)piperidine-1,4-dicarboxylate (8.40 g, 22.44 mmol), and dimethyl[2,2′-bipyridine]-4,4′-dicarboxylate (0.12 g, 0.45 mmol) in THF/DMA (160 mL, 1:1) were added NiBr₂(dme) (0.14 g, 0.45 mmol), ZnCl₂ (0.61 g, 4.48 mmol), and zinc dust (2.93 g, 44.88 mmol). The reaction mixture was stirred at room temperature for 16 h. After completion of the reaction, the reaction mixture was filtered through celite and washed with ethyl acetate (50 mL). The filtrate was evaporated to get a crude product which was purified by column chromatography (50% ethyl acetate in petroleum ether) to afford benzyl 4-(1-(tert-butoxycarbonyl)piperidine-4-carbonyl)piperidine-1-carboxylate (8.0 g)

LC-MS (ESI): m/z=453.62 [M+22+H]⁺

### Preparation of benzyl 4-(piperidine-4-carbonyl)piperidine-1-carboxylate

To a stirred solution of benzyl 4-(1-(tert-butoxycarbonyl)piperidine-4-carbonyl)piperidine-1-carboxylate (6.0 g, 13.93 mmol) in DCM (120 mL) was added TFA (30.0 mL) at 0° C. The mixture was stirred at room temperature for 5 h. After completion, the reaction mixture was concentrated under reduced pressure, then diluted with water (20 mL) and extracted with ethyl acetate (20 mL). After that, the aqueous layer was basified (pH=12) by saturated solution of sodium hydroxide (20 mL) and extracted with ethyl acetate (3×20 mL). The organic was evaporated to get crude benzyl 4-(piperidine-4-carbonyl)piperidine-1-carboxylate (4.0 g) as a pale yellow liquid which was used in the next step without further purification.

LC-MS (ESI): m/z=331.18 [M+H]

### Preparation of benzyl 4-(1-(3-(2,6-bis(benzyloxy)pyridin-3-yl)-1-methyl-1H-indazol-7-yl)piperidine-4-carbonyl)piperidine-1-carboxylate

A stirred solution of benzyl 4-(piperidine-4-carbonyl)piperidine-1-carboxylate (2.64 g, 7.99 mmol), 3-(2,6-bis(benzyloxy)pyridin-3-yl)-7-bromo-1-methyl-1H-indazole (2.0 g, 3.99 mmol) and Cs₂CO₃ (3.89 g, 11.99 mmol) in 1,4-dioxane (40 mL) was degassed with N₂ for 5 min. Afterwards, Pd-PEPPSI-iHEPT-Cl (0.19 g, 0.2 mmol) was added, and the reaction mixture was heated at 100° C. and stirred for 16 h. After completion, the reaction was filtered through celite, washed with ethyl acetate and evaporated to get a crude compound which was purified by column chromatography (50% ethyl acetate in petroleum ether) to afford benzyl 4-(1-(3-(2,6-bis(benzyloxy)pyridin-3-yl)-1-methyl-1H-indazol-7-yl)piperidine-4-carbonyl)piperidine-1-carboxylate (1.8 g) as a white solid.

LC-MS (ESI): m/z=750.68 [M+H]⁺

### Preparation of benzyl(S)-4-(1-(1-(3-(2,6-bis(benzyloxy)pyridin-3-yl)-1-methyl-1H-indazol-7-yl)piperidin-4-yl)-1-hydroxyethyl)piperidine-1-carboxylate and benzyl (R)-4-(1-(1-(3-(2,6-bis(benzyloxy)pyridin-3-yl)-1-methyl-1H-indazol-7-yl)piperidin-4-yl)-1-hydroxyethyl)piperidine-1-carboxylate

To a stirred solution of benzyl 4-(1-(3-(2,6-bis(benzyloxy)pyridin-3-yl)-1-methyl-1H-indazol-7-yl)piperidine-4-carbonyl)piperidine-1-carboxylate (2.0 g, 2.66 mmol) in THF (60 mL) was added methyl magnesium chloride (3.0 M in diethyl ether) (4.43 mL, 13.33 mmol) at −60° C. The mixture was allowed to stir at room temperature for 5 h. After completion, the reaction mixture was quenched with NH₄Cl (20 mL) and extracted with ethyl acetate (2×100 mL). The organic layer was evaporated to get a crude product which was purified by column chromatography (50% ethyl acetate in petroleum ether) to afford benzyl 4-(1-(1-(3-(2,6-bis(benzyloxy)pyridin-3-yl)-1-methyl-1H-indazol-7-yl)piperidin-4-yl)-1-hydroxyethyl)piperidine-1-carboxylate (1.8 g) as a white solid. Stereoisomers of benzyl 4-(1-(1-(3-(2,6-bis(benzyloxy)pyridin-3-yl)-1-methyl-1H-indazol-7-yl)piperidin-4-yl)-1-hydroxyethyl)piperidine-1-carboxylate were separated by SFC to provide benzyl(S)-4-(1-(1-(3-(2,6-bis(benzyloxy)pyridin-3-yl)-1-methyl-1H-indazol-7-yl)piperidin-4-yl)-1-hydroxyethyl)piperidine-1-carboxylate and benzyl (R)-4-(1-(1-(3-(2,6-bis(benzyloxy)pyridin-3-yl)-1-methyl-1H-indazol-7-yl)piperidin-4-yl)-1-hydroxyethyl)piperidine-1-carboxylate

LC-MS (ESI): m/z=766.67 [M+H]⁺

### Preparation of 3-(7-(4-((R)-1-hydroxy-1-(piperidin-4-yl)ethyl)piperidin-1-yl)-1-methyl-1H-indazol-3-yl)piperidine-2,6-dione

To a stirred solution of benzyl(S)-4-(1-(1-(3-(2,6-bis(benzyloxy)pyridin-3-yl)-1-methyl-1H-indazol-7-yl)piperidin-4-yl)-1-hydroxyethyl)piperidine-1-carboxylate (0.6 g, 0.78 mmol) in THF (18.0 mL) was added 20% palladium hydroxide on carbon (1.8 g, 100% w/w). The reaction mixture was stirred under H₂ pressure (80 psi) at room temperature for 16 h. After completion, the reaction mixture was filtered through celite and washed with DCM (50 mL). The filtrate was evaporated to get a crude compound which was triturated with diethyl ether to afford 3-(7-(4-((R)-1-hydroxy-1-(piperidin-4-yl)ethyl)piperidin-1-yl)-1-methyl-1H-indazol-3-yl)piperidine-2,6-dione (0.15 g) as a white solid.

### Preparation of 2-((6-((5-chloro-2-(4-((1S)-1-(1-(3-(2,6-dioxopiperidin-3-yl)-1-methyl-1H-indazol-7-yl)piperidin-4-yl)-1-hydroxyethyl)piperidin-1-yl)pyrimidin-4-yl)amino)-1-isopropyl-2-oxo-1,2-dihydro-1,8-naphthyridin-3-yl)oxy)-N-methylacetamide [rel-2-((6-((5-chloro-2-(4-((1S)-1-(1-(3-(2,6-dioxopiperidin-3-yl)-1-methyl-1H-indazol-7-yl)piperidin-4-yl)-1-hydroxyethyl)piperidin-1-yl)pyrimidin-4-yl)amino)-1-isopropyl-2-oxo-1,2-dihydro-1,8-naphthyridin-3-yl)oxy)-N-methylacetamide]

To a stirred solution of 3-(7-(4-((R)-1-hydroxy-1-(piperidin-4-yl)ethyl)piperidin-1-yl)-1-methyl-1H-indazol-3-yl)piperidine-2,6-dione (0.1 g, 0.22 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.058 g, 0.13 mmol) in DMSO (3.0 mL) was added DIPEA (0.4 mL, 2.20 mmol). The reaction mixture was heated at 100° C. and stirred for 5 h. The reaction mixture was concentrated under reduced pressure to get a crude product which was purified by prep-HPLC to afford 2-((6-((5-chloro-2-(4-((1S)-1-(1-(3-(2,6-dioxopiperidin-3-yl)-1-methyl-1H-indazol-7-yl)piperidin-4-yl)-1-hydroxyethyl)piperidin-1-yl)pyrimidin-4-yl)amino)-1-isopropyl-2-oxo-1,2-dihydro-1,8-naphthyridin-3-yl)oxy)-N-methylacetamide [rel-2-((6-((5-chloro-2-(4-((1S)-1-(1-(3-(2,6-dioxopiperidin-3-yl)-1-methyl-1H-indazol-7-yl)piperidin-4-yl)-1-hydroxyethyl)piperidin-1-yl)pyrimidin-4-yl)amino)-1-isopropyl-2-oxo-1,2-dihydro-1,8-naphthyridin-3-yl)oxy)-N-methylacetamide] (27.16 mg) as a white solid.

¹H NMR (400 MHz, DMSO-d₆): δ 10.87 (s, 1H), 9.02 (s 1H), 8.70 (d, J=2.4 Hz, 1H), 8.03 (d, J=2.4 Hz, 1H), 8.07 (s, 1H), 7.94 (d, J=4.8 Hz, 1H), 7.36 (dd, J=6.8, 1.6 Hz, 1H), 7.10 (s, 1H), 7.02-6.97 (m, 2H), 5.96 (bs, 1H), 4.57 (s, 4H), 4.34-4.31 (m, 1H), 4.24 (s, 3H), 3.93 (s, 1H), 3.31 (bs, 2H), 2.79-2.62 (m, 9H), 2.30 (d, J=5.2 Hz, 1H), 2.19-2.14 (m, 1H), 1.82-1.77 (m, 1H), 1.74-1.56 (m, 13H), 1.15-1.30 (m, 2H), 0.97 (s, 3H).

LC-MS (ESI): m/z=854.49 [M+H]⁺

### Example 77: 2-((6-((5-chloro-2-(4-((4-(3-(2,6-dioxopiperidin-3-yl)-1-methyl-1H-indazol-6-yl)-2,2-dimethylpiperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide (Compound 196)

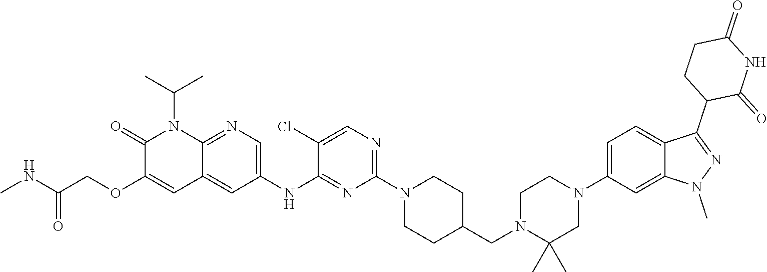 [SMILES: CNC(=O)COc1cc2cc(Nc3nc(N4CCC(CN5CCN(c6ccc7c(C8CCC(=O)NC8=O)nn(C)c7c6)CC5(C)C)CC4)ncc3Cl)cnc2n(C(C)C)c1=O]

### Preparation of tert-butyl 4-((1-((benzyloxy) carbonyl)piperidin-4-yl)methyl)-3,3-dimethylpiperazine-1-carboxylate

To a stirred solution of tert-butyl 3,3-dimethylpiperazine-1-carboxylate (1.0 g, 4.66 mmol) and benzyl 4-formylpiperidine-1-carboxylate (1.38 g, 5.60 mmol) in THF (10.0 ml) was added acetic acid (0.20 ml, 4.66 mmol) and anhydrous sodium acetate (1.14 g, 13.99 mmol). The reaction was stirred at room temperature for 1 h followed by the addition of sodium triacetoxyborohydride (2.96 g, 13.99 mmol) at 0° C. The reaction mixture was stirred at room temperature for 12 h. The reaction mixture was quenched with water (50 ml) and extracted with ethyl acetate (100 mL). The organic layer was separated and concentrated under reduced pressure to get crude product which was purified by flash column chromatography (25% ethyl acetate in petroleum ether) to afford tert-butyl 4-((1-((benzyloxy) carbonyl)piperidin-4-yl)methyl)-3,3-dimethylpiperazine-1-carboxylate as a pure product (1.0 g).

LC-MS (ESI): m/z=446.57 [M+H]

### Preparation of benzyl 4-((2,2-dimethylpiperazin-1-yl)methyl)piperidine-1-carboxylate

To a stirred solution of tert-butyl 4-((1-((benzyloxy) carbonyl)piperidin-4-yl)methyl)-3,3-dimethylpiperazine-1-carboxylate (2 g, 4.48 mmol) in DCM (20 mL) was added TFA (2 mL) at 0° C. The reaction mixture was stirred at room temperature for 3 h. The reaction mixture was concentrated under reduced pressure to get a crude product which was purified by trituration with diethyl ether (20 mL) to afford benzyl 4-((2,2-dimethylpiperazin-1-yl)methyl)piperidine-1-carboxylate (1.5 g) as a solid.

LC-MS (ESI): m/z=346.47 [M+H]⁺

### Preparation of benzyl 4-((4-(3-(2,6-bis(benzyloxy)pyridin-3-yl)-1-methyl-1H-indazol-7-yl)-2,2-dimethylpiperazin-1-yl)methyl)piperidine-1-carboxylate

To a stirred solution of benzyl 4-((2,2-dimethylpiperazin-1-yl)methyl)piperidine-1-carboxylate (1.0 g, 2.89 mmol), 3-(2,6-bis(benzyloxy)pyridin-3-yl)-6-bromo-1-methyl-1H-indazole (0.72 g, 1.44 mmol), and ^{t}BuONa (2 M in THF) (4.33 mL, 8.67 mmol) in 1-4 dioxane (10 mL) was added Ruphos-Pd-G3 (0.24 g, 0.28 mmol) under N₂. The reaction mixture was heated at 100° C. and stirred for 16 h. The reaction mixture was quenched with ice cold water (20 mL) and extracted with ethyl acetate (100 mL). The organic layer was separated and concentrated under reduced pressure to get a crude product which was purified by flash column chromatography (50-60% ethyl acetate in petroleum ether) to afford benzyl b 4-((4-(3-(2,6-bis(benzyloxy)pyridin-3-yl)-1-methyl-1H-indazol-7-yl)-2,2-dimethylpiperazin-1-yl)methyl)piperidine-1-carboxylate (0.53 g).

LC-MS (ESI): m/z=765.80 [M+H]⁺

### Preparation of 3-(6-(3,3-dimethyl-4-(piperidin-4-ylmethyl)piperazin-1-yl)-1-methyl-1H-indazol-3-yl)piperidine-2,6-dione

A stirred solution of benzyl 4-((4-(3-(2,6-bis(benzyloxy)pyridin-3-yl)-1-methyl-1H-indazol-7-yl)-2,2-dimethylpiperazin-1-yl)methyl)piperidine-1-carboxylate (0.80 g, 1.04 mmol) in THF (24 mL) was degassed with nitrogen for 5 min followed by the addition of 20% palladium hydroxide on carbon (0.80 g, 100% w/w). The mixture was stirred under hydrogen pressure (80 psi) at room temperature for 16 h. The reaction mixture was diluted with ethyl acetate (50 mL), filtered through a celite bed, and concentrated to get crude product which was purified by trituration with diethyl ether to afford 3-(6-(3,3-dimethyl-4-(piperidin-4-ylmethyl)piperazin-1-yl)-1-methyl-1H-indazol-3-yl)piperidine-2,6-dione (0.20 g) as a solid.

LC-MS (ESI): m/z=553.51 [M+H]⁺

### Preparation of 2-((6-((5-chloro-2-(4-((4-(3-(2,6-dioxopiperidin-3-yl)-1-methyl-1H-indazol-6-yl)-2,2-dimethylpiperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide

To a stirred solution of 3-(6-(3,3-dimethyl-4-(piperidin-4-ylmethyl)piperazin-1-yl)-1-methyl-1H-indazol-3-yl)piperidine-2,6-dione (0.100 g, 0.221 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.048 g, 0.110 mmol) in DMSO (2 mL) was added N,N-diisopropylethylamine (0.11 mL, 0.66 mmol) at room temperature. The mixture was heated at 100° C. and stirred for 4 h. The reaction mixture was concentrated under reduced pressure and purified by prep-HPLC to afford 2-((6-((5-chloro-2-(4-((4-(3-(2,6-dioxopiperidin-3-yl)-1-methyl-1H-indazol-6-yl)-2,2-dimethylpiperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide (0.022 g) as an off white solid.

¹H NMR (400 MHz, DMSO-d₆): 10.84 (s, 1H), 9.03 (s, 1H), 8.68 (s, 1H), 8.29 (s 1H), 8.07 (s 1H), 7.94 (bs, 1H), 7.47 (d, J=8.4 Hz, 1H), 7.08 (s, 1H), 6.96-6.80 (m, 2H), 5.95 (bs, 1H), 4.56-4.46 (m, 4H), 4.25 (s, 1H), 3.88 (s, 3H), 3.40-3.37 (m, 1H), 3.24 (bs, 2H), 3.04-2.81 (m, 4H), 2.66-2.50 (m, 6H), 2.18 (bs, 3H), 1.75 (m, 2H), 1.56 (d, J=6.8 Hz, 6H), 1.39 (bs, 1H), 1.22-0.90 (m, 9H).

LC-MS (ESI): m/z=853.50 [M+H]⁺

### Example 78: 2-((6-((5-chloro-2-((6S,7R)-7-((3-(2,6-dioxopiperidin-3-yl)-1-methyl-1H-indazol-6-yl)amino)-6-(trifluoromethyl)-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide [rel-2-((6-((5-chloro-2-((6S,7R)-7-((3-(2,6-dioxopiperidin-3-yl)-1-methyl-1H-indazol-6-yl)amino)-6-(trifluoromethyl)-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide] (Compound 221a)

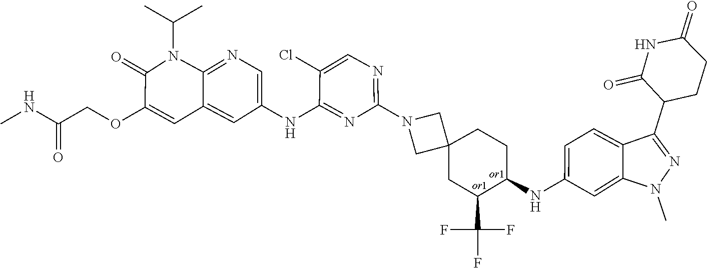 [SMILES: CNC(=O)COc1cc2cc(Nc3nc(N4CC5(CC[C@@H](Nc6ccc7c(C8CCC(=O)NC8=O)nn(C)c7c6)[C@@H](C(F)(F)F)C5)C4)ncc3Cl)cnc2n(C(C)C)c1=O]

### Preparation of tert-butyl (6S,7R)-7-[[3-(2,6-dibenzyloxy-3-pyridyl)-1-methyl-indazol-6-yl]amino]-6-(trifluoromethyl)-2-azaspiro[3.5]nonane-2-carboxylate

A mixture of 6-bromo-3-(2,6-dibenzyloxy-3-pyridyl)-1-methyl-indazole (400 mg, 799.38 μmol), (6S,7R)-7-amino-6-(trifluoromethyl)-2-azaspiro[3.5]nonane-2-carboxylate (295.78 mg, 959.26 μmol), Cs₂CO₃ (781.36 mg, 2.40 mmol), and XPhos Pd G3 (67.66 mg, 79.94 μmol) in dioxane (10 mL) was degassed and purged with N₂ three times. The mixture was stirred at 90° C. for 12 h under N₂ atmosphere. The reaction mixture was partitioned between H₂O (10 mL) and ethyl acetate (30 mL). The organic phase was separated, dried over Na₂SO₄, filtered, and concentrated under reduced pressure to give a residue. The residue was purified by flash silica gel chromatography (0~40% ethyl acetate/petroleum ether) to afford tert-butyl (6S,7R)-7-[[3-(2,6-dibenzyloxy-3-pyridyl)-1-methyl-indazol-6-yl]amino]-6-(trifluoromethyl)-2-azaspiro[3.5]nonane-2-carboxylate (250 mg, 291.97 μmol) as a blue oil.

LC-MS (ESI): m/z=633.5 [M+H]⁺.

### Preparation of tert-butyl (6S,7R)-7-[[3-(2,6-dioxo-3-piperidyl)-1-methyl-indazol-6-yl]amino]-6-(trifluoromethyl)-2-azaspiro[3.5]nonane-2-carboxylate

A mixture of tert-butyl (6S,7R)-7-[[3-(2,6-dibenzyloxy-3-pyridyl)-1-methyl-indazol-6-yl]amino]-6-(trifluoromethyl)-2-azaspiro[3.5]nonane-2-carboxylate (250 mg, 343.50 μmol), Pd/C (365.55 mg, 343.50 μmol, 10% purity), and Pd(OH)₂ (241.19 mg, 343.50 μmol, 20% purity) in CF₃CH₂OH (10 mL) was degassed and purged with H₂ three times. The mixture was stirred at 25° C. for 12 h under H₂ atmosphere. The reaction mixture was filtered, and the filtrate was concentrated to give a residue. The residue was purified by flash silica gel chromatography (0~10% DCM/MeOH) to afford tert-butyl (6S,7R)-7-[[3-(2,6-dioxo-3-piperidyl)-1-methyl-indazol-6-yl]amino]-6-(trifluoromethyl)-2-azaspiro[3.5]nonane-2-carboxylate (130 mg, 182.14 μmol) as a blue oil.

LC-MS (ESI): m/z=550.2 [M+H]⁺.

¹H NMR (400 MHz, DMSO-d₆) δ=10.92-10.77 (m, 1H), 8.83-8.74 (m, 1H), 8.24 (d, J=2.0 Hz, 1H), 8.05-7.99 (m, 1H), 7.81 (dd, J=2.0, 9.2 Hz, 1H), 7.55 (d, J=8.8 Hz, 1H), 7.42 (d, J=9.2 Hz, 1H), 6.85 (s, 1H), 6.67 (dd, J=2.0, 8.8 Hz, 1H), 6.28-6.10 (m, 1H), 4.83-4.68 (m, 1H), 4.52-4.33 (m, 2H), 4.29 (dd, J=5.2, 9.6 Hz, 1H), 4.06 (s, 2H), 4.00-3.94 (m, 2H), 3.93-3.88 (m, 3H), 3.56 (s, 3H), 2.88-2.76 (m, 2H), 2.69-2.55 (m, 2H), 2.37-2.20 (m, 3H), 2.19-2.08 (m, 1H), 2.03-1.87 (m, 1H), 1.40-1.21 (m, 1H), 0.79-0.62 (m, 1H), 0.59-0.45 (m, 2H), 0.43-0.29 (m, 1H)

### Preparation of 3-[1-methyl-6-[[(6S,7R)-6-(trifluoromethyl)-2-azaspiro[3.5]nonan-7-yl]amino] indazol-3-yl]piperidine-2,6-dione

A solution of tert-butyl (6S,7R)-7-[[3-(2,6-dioxo-3-piperidyl)-1-methyl-indazol-6-yl]amino]-6-(trifluoromethyl)-2-azaspiro[3.5]nonane-2-carboxylate (110 mg, 200.15 μmol) in DCM (1.5 mL) and TFA (0.5 mL) was stirred at 25° C. for 1 h. The reaction mixture was concentrated under reduced pressure to give 3-[1-methyl-6-[[(6S,7R)-6-(trifluoromethyl)-2-azaspiro[3.5]nonan-7-yl]amino] indazol-3-yl]piperidine-2,6-dione (89 mg, 189.99 μmol) as a blue oil.

LC-MS (ESI): m/z=250.2 [M+H]⁺.

### Preparation of 2-((6-((5-chloro-2-((6S,7R)-7-((3-(2,6-dioxopiperidin-3-yl)-1-methyl-1H-indazol-6-yl)amino)-6-(trifluoromethyl)-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide [rel-2-((6-((5-chloro-2-((6S,7R)-7-((3-(2,6-dioxopiperidin-3-yl)-1-methyl-1H-indazol-6-yl)amino)-6-(trifluoromethyl)-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide]

To a solution of 3-[1-methyl-6-[[(6S,7R)-6-(trifluoromethyl)-2-azaspiro[3.5]nonan-7-yl]amino] indazol-3-yl]piperidine-2,6-dione (40 mg, 88.99 μmol, 1 eq) and 2-[[6-[(2,5-dichloropyrimidin-4-yl)amino]-1-isopropyl-2-oxo-1,8-naphthyridin-3-yl]oxy]-N-methyl-acetamide (31.13 mg, 71.20 μmol) in DMSO (1 mL) was added Et₃N (36.02 mg, 355.98 μmol, 49.55 μL). The mixture was stirred at 100° C. for 1 hr. The reaction mixture was filtered, and the filtrate was concentrated to give a residue. The residue was purified by prep-HPLC to afford 2-((6-((5-chloro-2-((6S,7R)-7-((3-(2,6-dioxopiperidin-3-yl)-1-methyl-1H-indazol-6-yl)amino)-6-(trifluoromethyl)-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide [rel-2-((6-((5-chloro-2-((6S,7R)-7-((3-(2,6-dioxopiperidin-3-yl)-1-methyl-1H-indazol-6-yl)amino)-6-(trifluoromethyl)-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide] (15.68 mg, 16.72 μmol) as a yellow solid.

LC-MS (ESI): m/z=850.4 [M+H]⁺.

¹H NMR (400 MHz, DMSO-d₆) δ=11.04-10.50 (m, 1H), 9.17-8.95 (m, 1H), 8.80 (dd, J=1.6, 2.8 Hz, 1H), 8.58-8.14 (m, 1H), 8.07 (s, 1H), 7.46-7.27 (m, 1H), 7.19 (s, 1H), 6.75-6.60 (m, 1H), 6.58-6.40 (m, 1H), 6.06-5.90 (m, 1H), 5.86-5.79 (m, 1H), 4.69-4.47 (m, 2H), 4.23-4.03 (m, 2H), 3.87-3.79 (m, 4H), 3.79-3.74 (m, 2H), 3.74-3.68 (m, 1H), 2.73-2.64 (m, 4H), 2.63-2.56 (m, 2H), 2.35-2.21 (m, 3H), 2.17-2.08 (m, 1H), 2.04-1.89 (m, 2H), 1.87-1.79 (m, 2H), 1.73-1.65 (m, 1H), 1.57 (d, J=7.2 Hz, 6H)

### Example 79: 2-((6-((5-chloro-2-(4-((1r,3r)-3-(4-(3-(2,6-dioxopiperidin-3-yl)-1-methyl-1H-indazol-6-yl)piperazin-1-yl)cyclobutoxy)piperidin-1-yl)pyrimidin-4-yl)amino)-1-isopropyl-2-oxo-1,2-dihydro-1,8-naphthyridin-3-yl)oxy)-N-methylacetamide (Compound 226a)

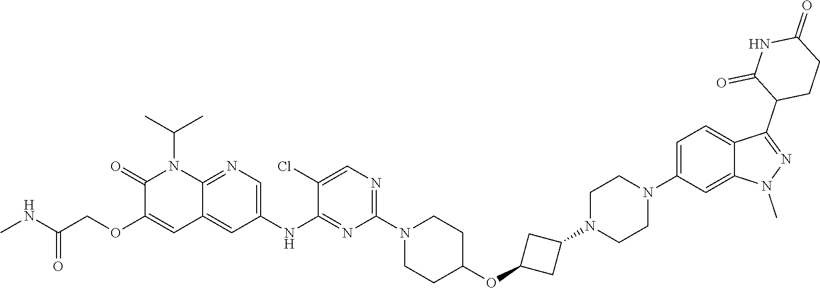 [SMILES: CNC(=O)COc1cc2cc(Nc3nc(N4CCC(O[C@H]5C[C@H](N6CCN(c7ccc8c(C9CCC(=O)NC9=O)nn(C)c8c7)CC6)C5)CC4)ncc3Cl)cnc2n(C(C)C)c1=O]

### Preparation of tert-butyl 4-((1r,3r)-3-(piperazin-1-yl)cyclobutoxy)piperidine-1-carboxylate

To a stirred solution of benzyl 4-((1r,3r)-3-((1-(tert-butoxycarbonyl)piperidin-4-yl)oxy) cyclobutyl)piperazine-1-carboxylate (1.7 g, 3.58 mmol) in THF (68 mL) was added 20% Pd(OH)₂ (2.01 g, 14.35 mmol). The mixture was stirred under hydrogen atmosphere (80 psi) at room temperature for 16 h. The reaction mixture was diluted with THF and filtered through a pad of celite, which was washed with excess of 20% THF in DCM. The filtrate was collected and concentrated to afford tert-butyl 4-((1r,3r)-3-(piperazin-1-yl)cyclobutoxy)piperidine-1-carboxylate (1.2 g) as a yellow semi-solid.

LC-MS (ESI): m/z=340.33 [M+H]⁺

### Preparation of tert-butyl 4-((1r,3r)-3-(4-(3-(2,6-bis(benzyloxy)pyridin-3-yl)-1-methyl-1H-indazol-6-yl)piperazin-1-yl)cyclobutoxy)piperidine-1-carboxylate

To a stirred solution of 3-(2,6-bis(benzyloxy)pyridin-3-yl)-6-bromo-1-methyl-1H-indazole (1.0 g, 1.99 mmol) and tert-butyl 4-((1r,3r)-3-(piperazin-1-yl)cyclobutoxy)piperidine-1-carboxylate (1.14 g, 3.38 mmol) in 1,4-dioxane (10.0 mL) was added potassium tert-butoxide (0.66 g, 5.97 mmol). The mixture was purged with argon for 5 minutes followed by the addition of bis(tri-tert-butylphosphine) palladium (0) (0.10 g, 0.19 mmol). The resultant reaction mixture was heated at 100° C. for 16 h. The reaction was filtered through a celite pad and the filtrate was extracted with ethyl acetate (20 mL×2). The combined organic layer was washed with brine (10 mL), dried over sodium sulfate, filtered, and concentrated under reduced pressure to get the crude product which was purified by flash column chromatography (10% methanol in DCM) to afford tert-butyl 4-((1r,3r)-3-(4-(3-(2,6-bis(benzyloxy)pyridin-3-yl)-1-methyl-1H-indazol-6-yl)piperazin-1-yl)cyclobutoxy)piperidine-1-carboxylate (1.0 g) as a brown semi-solid.

LC-MS (ESI): m/z=759.36 [M+H]⁺

### Preparation of tert-butyl 4-((1r,3r)-3-(4-(3-(2,6-dioxopiperidin-3-yl)-1-methyl-1H-indazol-6-yl)piperazin-1-yl)cyclobutoxy)piperidine-1-carboxylate

To a stirred solution of tert-butyl 4-((1r,3r)-3-(4-(3-(2,6-bis(benzyloxy)pyridin-3-yl)-1-methyl-1H-indazol-6-yl)piperazin-1-yl)cyclobutoxy)piperidine-1-carboxylate (1.0 g, 1.31 mmol) in THF (40 mL) was added 20% Pd(OH)₂, (0.74 g, 5.27 mmol). The mixture was stirred under hydrogen atmosphere (80 psi) at room temperature for 16 h. The reaction mixture was diluted with THF and filtered through a pad of celite, which was washed with an excess of 20% THF in DCM. The filtrate was collected and concentrated to afford tert-butyl 4-((1r,3r)-3-(4-(3-(2,6-dioxopiperidin-3-yl)-1-methyl-1H-indazol-6-yl)piperazin-1-yl) cyclobutoxy)piperidine-1-carboxylate (0.75 g) as a yellow semi-solid.

LC-MS (ESI): m/z=581.77 [M+H]⁺

### Preparation of 3-(1-methyl-6-(4-((1r,3r)-3-(piperidin-4-yloxy)cyclobutyl)piperazin-1-yl)-1H-indazol-3-yl)piperidine-2,6-dione

To a stirred solution of tert-butyl 4-((1r,3r)-3-(4-(3-(2,6-dioxopiperidin-3-yl)-1-methyl-1H-indazol-6-yl)piperazin-1-yl)cyclobutoxy)piperidine-1-carboxylate (0.75 g, 1.29 mmol) in DCM (15 mL) was added TFA (0.69 g, 2.58 mmol). The mixture was stirred at room temperature for 2 h. The reaction mixture was concentrated under reduced pressure to get a crude product which was triturated with diethyl ether (5 mL). The solid was dried to afford 3-(1-methyl-6-(4-((1r,3r)-3-(piperidin-4-yloxy)cyclobutyl)piperazin-1-yl)-1H-indazol-3-yl)piperidine-2,6-dione (0.6 g, crude) as a black semi-solid.

LC-MS (ESI): m/z=481.49 [M+H]⁺

### Preparation of 2-((6-((5-chloro-2-(4-((1r,3r)-3-(4-(3-(2,6-dioxopiperidin-3-yl)-1-methyl-1H-indazol-6-yl)piperazin-1-yl)cyclobutoxy)piperidin-1-yl)pyrimidin-4-yl)amino)-1-isopropyl-2-oxo-1,2-dihydro-1,8-naphthyridin-3-yl)oxy)-N-methylacetamide

To a stirred solution of 3-(1-methyl-6-(4-((1r,3r)-3-(piperidin-4-yloxy)cyclobutyl)piperazin-1-yl)-1H-indazol-3-yl)piperidine-2,6-dione (0.3 g, 0.62 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.08 g, 0.18 mmol) in DMSO (3.0 mL) was added N,N-diisopropylethylamine (0.89 mL, 4.99 mmol). The reaction mixture was stirred at 100° C. for 6 h. The reaction mixture was quenched with ice-cold water, and the solid precipitate was filtered and dried under vacuum to get a crude product which was purified by prep-HPLC to afford 2-((6-((5-chloro-2-(4-((1r,3r)-3-(4-(3-(2,6-dioxopiperidin-3-yl)-1-methyl-1H-indazol-6-yl)piperazin-1-yl)cyclobutoxy)piperidin-1-yl)pyrimidin-4-yl)amino)-1-isopropyl-2-oxo-1,2-dihydro-1,8-naphthyridin-3-yl)oxy)-N-methylacetamide (0.053 g) as a brown solid.

LC-MS (ESI): m/z=881.41 [M+H]⁺

¹H NMR (400 MHz, DMSO-d₆): δ 10.90 (s, 1H), 9.06 (s, 1H), 8.66 (d, J=1.60 Hz, 1H), 8.29 (d, J=2.00 Hz, 1H), 8.18 (s, 2H), 8.08 (s, 1H), 7.94 (d, J=4.40 Hz, 1H), 7.49 (d, J=8.80 Hz, 1H), 7.08 (s, 1H), 6.90 (d, J=8.80 Hz, 1H), 6.83 (s, 1H), 5.96 (br s, 1H), 4.57 (s, 2H), 4.27-4.23 (m, 1H), 4.06-4.03 (m, 2H), 3.88-3.86 (m, 4H), 3.54-3.52 (m, 1H), 3.33-3.21 (m, 7H), 2.68 (d, J=4.8 Hz, 3H), 2.63-2.54 (m, 3H), 2.50-2.42 (m, 5H), 2.35-2.33 (m, 1H), 2.18-2.11 (m, 1H), 1.81-1.69 (m, 4H), 1.57 (d, J=6.8 Hz, 6H), 1.39-1.36 (m, 2H).

### Example 80: 2-((6-((5-chloro-2-(4-(((3R)-1-(3-(2,6-dioxopiperidin-3-yl)-1-methyl-1H-indazol-6-yl)pyrrolidin-3-yl)methyl)piperazin-1-yl)pyrimidin-4-yl)amino)-1-isopropyl-2-oxo-1,2-dihydro-1,8-naphthyridin-3-yl)oxy)-N-methylacetamide (Compound 228b)

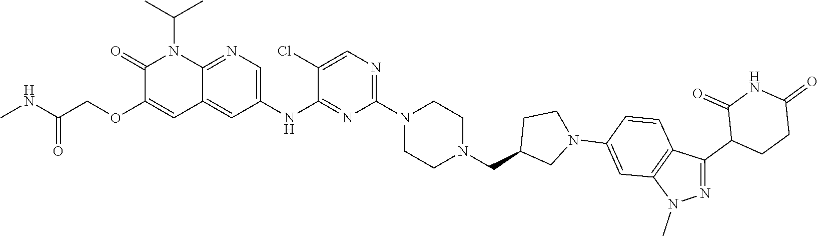 [SMILES: CNC(=O)COc1cc2cc(Nc3nc(N4CCN(C[C@H]5CCN(c6ccc7c(C8CCC(=O)NC8=O)nn(C)c7c6)C5)CC4)ncc3Cl)cnc2n(C(C)C)c1=O]

### Preparation of 1-(tert-butyl) 3-methyl(S)-pyrrolidine-1,3-dicarboxylate

To a stirred solution of(S)-1-(tert-butoxycarbonyl)pyrrolidine-3-carboxylic acid (5.0 g, 23.24 mmol) in methanol (150 mL) was added conc. H₂SO₄ dropwise at 0° C. The mixture was stirred at 80° C. for 3 h. The mixture was concentrated under reduced pressure, diluted with ethyl acetate (200 mL), and washed with a solution of sodium bicarbonate (300 mL) in water and brine (200 mL). The organic layers were dried over anhydrous Na₂SO₄, filtered, and concentrated to afford 1-(tert-butyl) 3-methyl(S)-pyrrolidine-1,3-dicarboxylate (5.0 g) as an off white semi-solid.

LC-MS (ESI): m/z=130.03 [M−100]⁺

### Preparation of methyl(S)-pyrrolidine-3-carboxylate

To a stirred solution of 1-(tert-butyl) 3-methyl(S)-pyrrolidine-1,3-dicarboxylate (5.0 g, 21.82 mmol) in DCM (50.0 mL) was added 4 M HCl in 1,4 dioxane (10.0 mL) at 0° C. The mixture was stirred at room temperature for 2 h. The reaction mixture was concentrated under reduced pressure, and the crude product was triturated with diethyl ether (50 mL) to afford methyl(S)-pyrrolidine-3-carboxylate (4.1 g) as a brown semi-solid.

LC-MS (ESI): m/z=130.03 [M+H]⁺

### Preparation of methyl(S)-1-(3-(2,6-bis(benzyloxy)pyridin-3-yl)-1-methyl-1H-indazol-6-yl)pyrrolidine-3-carboxylate

To a stirred solution of methyl(S)-pyrrolidine-3-carboxylate (3.0 g, 23.2 mmol) and 3-(2,6-bis(benzyloxy)pyridin-3-yl)-6-bromo-1-methyl-1H-indazole (5.80 g, 11.6 mmol) in dioxane was added cesium carbonate (22.6 g, 69.6 mmol). The reaction mixture was degassed with argon for 10 minutes. To this mixture was added Ruphos Pd-G₃ (0.9 g, 1.1 mmol), and the resulting mixture was again degassed for 5 minutes. The mixture was stirred at 100° C. for 16 h. The reaction mixture was filtered through celite and concentrated under vacuum to obtain a crude product which was purified by column chromatography (50% ethyl acetate in petroleum ether) to afford methyl(S)-1-(3-(2,6-bis(benzyloxy)pyridin-3-yl)-1-methyl-1H-indazol-6-yl)pyrrolidine-3-carboxylate (1.3 g) as a pale yellow semi-solid.

LC-MS (ESI): m/z=549.54 [M+H]⁺

### Preparation of(S)-1-(3-(2,6-bis(benzyloxy)pyridin-3-yl)-1-methyl-1H-indazol-6-yl)pyrrolidine-3-carboxylic acid

To a stirred solution of methyl(S)-1-(3-(2,6-bis(benzyloxy)pyridin-3-yl)-1-methyl-1H-indazol-6-yl)pyrrolidine-3-carboxylate (1.3 g, 2.37 mmol) in THF (13 mL) and H₂O (6.5 mL) was added LiOH·H₂O (0.50 g, 11.85) at 0° C. The mixture was stirred at room temperature for 2 h. The reaction mixture was diluted with water (20 mL) and extracted with ethyl acetate (50 mL×2). The organic layer was dried over sodium sulphate and concentrated under reduced pressure to afford(S)-1-(3-(2,6-bis(benzyloxy)pyridin-3-yl)-1-methyl-1H-indazol-6-yl)pyrrolidine-3-carboxylic acid (1.2 g) as an off-white solid.

LC-MS (ESI): m/z=535.41 [M+H]⁺

### Preparation of tert-butyl (R)-4-(1-(3-(2,6-bis(benzyloxy)pyridin-3-yl)-1-methyl-1H-indazol-6-yl)pyrrolidine-3-carbonyl)piperazine-1-carboxylate

To a stirred solution of(S)-1-(3-(2,6-bis(benzyloxy)pyridin-3-yl)-1-methyl-1H-indazol-6-yl)pyrrolidine-3-carboxylic acid (1.10 g, 2.05 mmol) and tert-butyl piperazine-1-carboxylate (0.38 g, 2.05 mmol) in DMF (22.0 mL) was added N,N-diisopropylethylamine (1.07 mL, 6.15 mmol). The mixture was stirred at room temperature for 5 min followed by the addition of HATU (1.16 g, 3.07 mmol) at 0° C. The mixture was stirred at room temperature for 2 h. The reaction mixture was poured into ice-cold water and stirred for 10 min. The precipitated solid was filtered and dried to afford tert-butyl (R)-4-(1-(3-(2,6-bis(benzyloxy)pyridin-3-yl)-1-methyl-1H-indazol-6-yl)pyrrolidine-3-carbonyl)piperazine-1-carboxylate (1.24 g) as an off-white solid.

LC-MS (ESI): m/z=703.50 [M+H]⁺

### Preparation of tert-butyl (R)-4-((1-(3-(2,6-bis(benzyloxy)pyridin-3-yl)-1-methyl-1H-indazol-6-yl)pyrrolidin-3-yl)methyl)piperazine-1-carboxylate

To a stirred solution of tert-butyl (R)-4-(1-(3-(2,6-bis(benzyloxy)pyridin-3-yl)-1-methyl-1H-indazol-6-yl)pyrrolidine-3-carbonyl)piperazine-1-carboxylate (1.22 g, 1.73 mmol) in THF (24.4 mL) was added borane dimethylsulfide (neat) (12.2 mL) at 0° C. and stirred at room temperature for 16 h. The reaction mixture was quenched with methanol and evaporated under reduced pressure. Then, the residue was dissolved in methanol and refluxed at 50° C. for 2 h. The reaction mixture was concentrated under reduced pressure to get a crude product which was purified by column chromatography (70% ethyl acetate in petroleum ether) to afford tert-butyl (R)-4-((1-(3-(2,6-bis(benzyloxy)pyridin-3-yl)-1-methyl-1H-indazol-6-yl)pyrrolidin-3-yl)methyl)piperazine-1-carboxylate (0.9 g) as a brown semi-solid.

LC-MS (ESI): m/z=689.67 [M+H]⁺

### Preparation of tert-butyl 4-(((3R)-1-(3-(2,6-dioxopiperidin-3-yl)-1-methyl-1H-indazol-6-yl)pyrrolidin-3-yl)methyl)piperazine-1-carboxylate

To a stirred solution of tert-butyl (R)-4-((1-(3-(2,6-bis(benzyloxy)pyridin-3-yl)-1-methyl-1H-indazol-6-yl)pyrrolidin-3-yl)methyl)piperazine-1-carboxylate (0.86 g, 1.24 mmol) in DMF (25.8 mL) and AcOH (0.86 mL) was added 20% Pd(OH)₂ (1.72 g, 200% w/w). The mixture was stirred under hydrogen pressure (20 psi) at room temperature for 16 h. The reaction mixture was diluted with THF (100 mL), filtered through celite, and washed with THF (200 mL). The filtrate was concentrated and dried to afford tert-butyl 4-(((3R)-1-(3-(2,6-dioxopiperidin-3-yl)-1-methyl-1H-indazol-6-yl)pyrrolidin-3-yl)methyl)piperazine-1-carboxylate (0.60 g) as a brown semi-solid.

LC-MS (ESI): m/z=511.50 [M+H]⁺

### Preparation of 3-(1-methyl-6-((R)-3-(piperazin-1-ylmethyl)pyrrolidin-1-yl)-1H-indazol-3-yl)piperidine-2,6-dione

To a stirred solution of tert-butyl 4-(((3R)-1-(3-(2,6-dioxopiperidin-3-yl)-1-methyl-1H-indazol-6-yl)pyrrolidin-3-yl)methyl)piperazine-1-carboxylate (0.60 g, 1.17 mmol) in DCM (6.0 mL) was added TFA (3.0 mL) at 0° C. The mixture was stirred at room temperature for 2 h. The reaction mixture was concentrated and co-distilled with DCM (3×50 mL) to get a crude product which was triturated with diethyl ether (15 mL). The resulting product was dried to afford 3-(1-methyl-6-((R)-3-(piperazin-1-ylmethyl)pyrrolidin-1-yl)-1H-indazol-3-yl)piperidine-2,6-dione (0.5 g) as a brown solid.

LC-MS (ESI): m/z=411.21 [M+H]⁺

### Preparation of 2-((6-((5-chloro-2-(4-(((3R)-1-(3-(2,6-dioxopiperidin-3-yl)-1-methyl-1H-indazol-6-yl)pyrrolidin-3-yl)methyl)piperazin-1-yl)pyrimidin-4-yl)amino)-1-isopropyl-2-oxo-1,2-dihydro-1,8-naphthyridin-3-yl)oxy)-N-methylacetamide

To a stirred solution of 3-(1-methyl-6-((R)-3-(piperazin-1-ylmethyl)pyrrolidin-1-yl)-1H-indazol-3-yl)piperidine-2,6-dione (0.25 g, 0.60 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.079 g, 0.18 mmol) in DMSO (5.0 mL) was added N,N-diisopropylethylamine (0.55 mL, 3.0 mmol). The resulting reaction mixture was stirred at 100° C. for 6 h. The reaction mixture was poured into ice cold water (50 mL). The solid precipitated was filtered and dried to get a crude product which was purified by Prep-HPLC to afford 2-((6-((5-chloro-2-(4-(((3R)-1-(3-(2,6-dioxopiperidin-3-yl)-1-methyl-1H-indazol-6-yl)pyrrolidin-3-yl)methyl)piperazin-1-yl)pyrimidin-4-yl)amino)-1-isopropyl-2-oxo-1,2-dihydro-1,8-naphthyridin-3-yl)oxy)-N-methylacetamide (0.026 g) as an off-white solid.

LC-MS (ESI): m/z=811.66 [M+H]⁺

¹H NMR (400 MHz, DMSO-d₆): δ 10.83 (s, 1H), 9.08 (s, 1H), 8.67 (d, J=2.40 Hz, 1H), 8.31 (d, J=2.40 Hz, 1H), 8.10 (s, 1H), 7.93-7.92 (d, J=4.40 Hz, 1H), 7.46-7.44 (d, J=4.40 Hz, 1H), 7.11 (s, 1H), 6.54 (d, J=9.20 Hz, 1H), 6.34 (s, 1H), 5.88 (br, 1H), 4.58 (s, 2H), 4.24-4.23 (m, 1H), 3.84 (s, 3H), 3.69 (br, 4H), 3.64-3.49 (m, 1H), 3.49-3.47 (m, 2H), 3.08-3.07 (m, 1H), 2.67-2.59 (m, 7H), 2.40-2.39 (m, 4H), 2.25-2.18 (m, 4H), 1.74-1.73 (m, 1H), 1.56-1.55 (d, J=6.80 Hz, 6H).

### Example 130: 2-((6-((5-chloro-2-(4-((4-(3-(2,6-dioxopiperidin-3-yl)-1-methyl-1H-indazol-6-yl)piperidin-1-yl)methyl)piperidin-1-yl)pyrimidin-4-yl)amino)-1-isopropyl-2-oxo-1,2-dihydro-1,8-naphthyridin-3-yl)oxy)-N-methylacetamide (Compound 212)

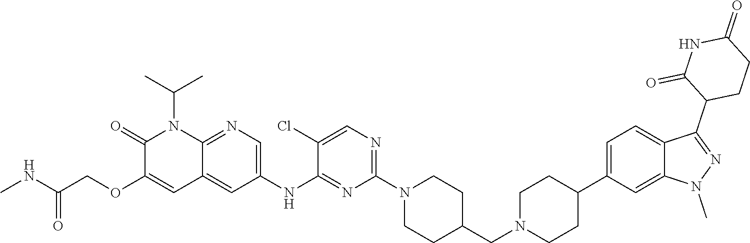 [SMILES: CNC(=O)COc1cc2cc(Nc3nc(N4CCC(CN5CCC(c6ccc7c(C8CCC(=O)NC8=O)nn(C)c7c6)CC5)CC4)ncc3Cl)cnc2n(C(C)C)c1=O]

### Preparation of tert-butyl 4-((4-(3-(2,6-dioxopiperidin-3-yl)-1-methyl-1H-indazol-6-yl)piperidin-1-yl)methyl)piperidine-1-carboxylate

To a stirred solution of 3-(1-methyl-6-(piperidin-4-yl)-1H-indazol-3-yl)piperidine-2,6-dione (0.60 g, 1.83 mmol, prepared using published methods e.g., in International Application Publication No. WO2021255212) and tert-butyl 4-formylpiperidine-1-carboxylate (0.62 g, 2.94 mmol) in THF (12 mL) was added acetic acid (0.6 mL) and sodium acetate (0.45 g, 5.49 mmol). The mixture was stirred for 30 min followed by the addition of sodium triacetoxy borohydride (1.16 g, 5.49 mmol) at 0° C. under nitrogen atmosphere. The mixture was stirred at room temperature for 2 h. The reaction mixture was diluted with water (200 mL) and extracted with ethyl acetate (2×100 mL). The combined organic layers were washed with brine, dried, and concentrated under reduced pressure to get a crude product which was purified by flash column chromatography (25% ethyl acetate in petroleum ether) to afford tert-butyl 4-((4-(3-(2,6-dioxopiperidin-3-yl)-1-methyl-1H-indazol-6-yl)piperidin-1-yl)methyl)piperidine-1-carboxylate (0.55 g) as a yellow semi-solid.

LC-MS (ESI): m/z=524.50 [M+H]⁺

### Preparation of 3-(1-methyl-6-(1-(piperidin-4-ylmethyl)piperidin-4-yl)-1H-indazol-3-yl)piperidine-2,6-dione

To a stirred solution of tert-butyl 4-((4-(3-(2,6-dioxopiperidin-3-yl)-1-methyl-1H-indazol-6-yl)piperidin-1-yl)methyl)piperidine-1-carboxylate (0.55 g, 1.50 mmol) in DCM (4.0 mL) was added TFA (4.0 mL) at 0° C. The mixture was stirred at room temperature for 1 h. The reaction mixture was concentrated and co-distilled with petroleum ether under reduced pressure to get a crude product which was triturated with diethyl ether to afford 3-(1-methyl-6-(1-(piperidin-4-ylmethyl)piperidin-4-yl)-1H-indazol-3-yl)piperidine-2,6-dione (0.55 g).

LC-MS (ESI): m/z=424.26 [M+H]⁺

### Preparation of 2-((6-((5-chloro-2-(4-((4-(3-(2,6-dioxopiperidin-3-yl)-1-methyl-1H-indazol-6-yl)piperidin-1-yl)methyl)piperidin-1-yl)pyrimidin-4-yl)amino)-1-isopropyl-2-oxo-1,2-dihydro-1,8-naphthyridin-3-yl)oxy)-N-methylacetamide

To a stirred solution of 3-(1-methyl-6-(1-(piperidin-4-ylmethyl)piperidin-4-yl)-1H-indazol-3-yl)piperidine-2,6-dione (0.55 g, 1.29 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.22 g, 0.51 mmol) in DMSO (11 mL) was added N,N-diisopropylethylamine (0.83 g, 6.45 mmol). The mixture was stirred at 100° C. for 16 h. The reaction mixture was poured into ice-cold water and stirred for 15 minutes. The resulting precipitate was filtered and dried to get a crude product which was purified by prep-HPLC to afford 2-((6-((5-chloro-2-(4-((4-(3-(2,6-dioxopiperidin-3-yl)-1-methyl-1H-indazol-6-yl)piperidin-1-yl)methyl)piperidin-1-yl)pyrimidin-4-yl)amino)-1-isopropyl-2-oxo-1,2-dihydro-1,8-naphthyridin-3-yl)oxy)-N-methylacetamide (78 mg) as a white solid.

LC-MS (ESI): m/z=822.41 [M−H]⁻

¹H NMR (400 MHz, DMSO-d₆): δ 10.87 (s, 1H), 9.03 (s, 1H), 8.69 (d, J=2.40 Hz, 1H), 8.30 (d, J=2.40 Hz, 1H), 8.07 (s, 1H), 7.95 (d, J=4.80 Hz, 1H), 7.60 (d, J=8.40 Hz, 1H), 7.44 (s, 1H), 7.10 (s, 1H), 7.03 (d, J=8.40 Hz, 1H), 5.94 (br s, 1H), 4.59 (s, 2H), 4.47-4.44 (m, 2H), 4.34-4.33 (m, 1H), 3.97 (s, 3H), 3.04-2.97 (m, 2H), 2.89-2.85 (m, 2H), 2.69-2.55 (m, 6H), 2.37-2.34 (s, 1H), 22.17-2.14 (m, 3H), 2.09-1.95 (m, 2H), 1.81-1.78 (m, 7H), 1.60 (d, J=4.80 Hz, 6H), 1.06-1.04 (m, 2H).

### Example 153: 2-((6-((5-chloro-2-(7-((1-(3-(2,6-dioxopiperidin-3-yl)-1-methyl-1H-indazol-7-yl)piperidin-4-yl)methyl)-3-oxa-7,9-diazabicyclo[3.3.1]nonan-9-yl)pyrimidin-4-yl)amino)-1-isopropyl-2-oxo-1,2-dihydro-1,8-naphthyridin-3-yl)oxy)-N-methylacetamide (Compound 162)

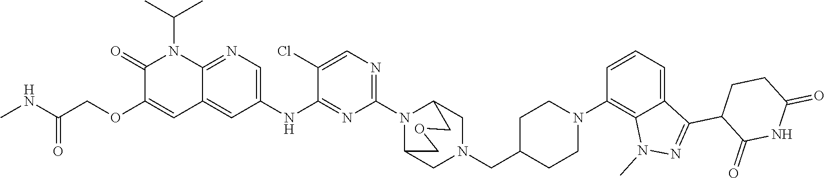 [SMILES: CNC(=O)COc1cc2cc(Nc3nc(N4C5COCC4CN(CC4CCN(c6cccc7c(C8CCC(=O)NC8=O)nn(C)c67)CC4)C5)ncc3Cl)cnc2n(C(C)C)c1=O]

### Preparation of tert-butyl 7-((1-((benzyloxy) carbonyl)piperidin-4-yl)methyl)-3-oxa-7,9-diazabicyclo[3.3.1]nonane-9-carboxylate

A solution of tert-butyl 3-oxa-7,9-diazabicyclo[3.3.1]nonane-9-carboxylate (1.03 g, 4.51 mmol) and benzyl 4-formylpiperidine-1-carboxylate (1.12 g, 4.51 mmol) in THF (22.5 mL) was treated with acetic acid (258 μL, 4.51 mmol), followed by sodium triacetoxyborohydride (1.43 g, 6.77 mmol). The reaction mixture was stirred at room temperature for 3 days. The reaction mixture was diluted with water and extracted ethyl acetate (3×). The combined organics were dried over Na₂SO₄, filtered, and concentrated under reduced pressure. The residue was purified by flash column chromatography (20-100% ethyl acetate in heptanes) to give tert-butyl 7-((1-((benzyloxy) carbonyl)piperidin-4-yl)methyl)-3-oxa-7,9-diazabicyclo[3.3.1]nonane-9-carboxylate (1.753 g) as a colorless oil. LC-MS (ESI): m/z=460.1 [M+H]⁺.

### Preparation of tert-butyl 7-(piperidin-4-ylmethyl)-3-oxa-7,9-diazabicyclo[3.3.1]nonane-9-carboxylate

A solution of tert-butyl 7-((1-((benzyloxy) carbonyl)piperidin-4-yl)methyl)-3-oxa-7,9-diazabicyclo[3.3.1]nonane-9-carboxylate (1.75 g, 3.81 mmol) in isopropanol (20 mL) and ethyl acetate (10 mL) was treated with Pd/C (405 mg, 10 wt %, 381 μmol). Hydrogen gas was bubbled through the reaction mixture several minutes, and the reaction mixture was stirred at room temperature under hydrogen atmosphere (balloon) overnight. The reaction mixture was filtered through a pad of celite, rinsing with ethyl acetate. The filtrate was evaporated to give tert-butyl 7-(piperidin-4-ylmethyl)-3-oxa-7,9-diazabicyclo[3.3.1]nonane-9-carboxylate (1.275 g, 97% purity) as a colorless oil, which was carried forward without additional purification.

LC-MS (ESI): m/z=326.4 [M+H]⁺.

### Preparation of tert-butyl 7-((1-(3-(2,6-bis(benzyloxy)pyridin-3-yl)-1-methyl-1H-indazol-7-yl)piperidin-4-yl)methyl)-3-oxa-7,9-diazabicyclo[3.3.1]nonane-9-carboxylate

To a stirred solution of tert-butyl 7-(piperidin-4-ylmethyl)-3-oxa-7,9-diazabicyclo[3.3.1]nonane-9-carboxylate (1.275 g, 97 wt %, 3.800 mmol) and 3-(2,6-bis(benzyloxy)pyridin-3-yl)-7-bromo-1-methyl-1H-indazole (2.282 g, 4.560 mmol) in THF (20 mL) under a nitrogen atmosphere was added RuPhos Pd G3 (317.8 mg, 380.0 μmol), followed by potassium tert-butoxide (11.40 mL, 11.40 mmol, 1.0 M in THF). The reaction mixture was equipped with a condenser and stirred under nitrogen atmosphere at 70° C. for 3 h 45 min. The reaction mixture was quenched with formic acid (573.4 μL, 15.20 mmol), then was concentrated under reduced pressure. The residue was taken up in DMSO and filtered, first through a pad of Celite® and then through a 0.45 μm hydrophilic PTFE filter. The filtrate was purified by preparative HPLC to give tert-butyl 7-((1-(3-(2,6-bis(benzyloxy)pyridin-3-yl)-1-methyl-1H-indazol-7-yl)piperidin-4-yl)methyl)-3-oxa-7,9-diazabicyclo[3.3.1]nonane-9-carboxylate (865.8 mg) as a tan solid. LC-MS (ESI): m/z=745.3 [M+H]⁺.

### Preparation of tert-butyl 7-((1-(3-(2,6-dioxopiperidin-3-yl)-1-methyl-1H-indazol-7-yl)piperidin-4-yl)methyl)-3-oxa-7,9-diazabicyclo[3.3.1]nonane-9-carboxylate

A solution of tert-butyl 7-((1-(3-(2,6-bis(benzyloxy)pyridin-3-yl)-1-methyl-1H-indazol-7-yl)piperidin-4-yl)methyl)-3-oxa-7,9-diazabicyclo[3.3.1]nonane-9-carboxylate (865.8 mg, 1.162 mmol) in isopropanol (10 mL)/DMF (2 mL)/ethyl acetate (2 mL) was treated with palladium hydroxide on carbon (204.0 mg, 20 wt %, 290.6 μmol). Hydrogen gas was bubbled through the reaction mixture with a long needle for several minutes, and the reaction mixture was then stirred under hydrogen atmosphere (balloon) at 50° C. in a heating block for two nights. The reaction mixture was filtered through a pad of celite, rinsing with ethyl acetate. The filtrate was concentrated under reduced pressure. The reaction mixture was taken up in DMSO and filtered through a 0.45 μm hydrophilic PTFE filter. The filtrate was purified by preparative HPLC to give tert-butyl 7-((1-(3-(2,6-dioxopiperidin-3-yl)-1-methyl-1H-indazol-7-yl)piperidin-4-yl)methyl)-3-oxa-7,9-diazabicyclo[3.3.1]nonane-9-carboxylate (303.2 mg) as a grey semi-solid. LC-MS (ESI): m/z=567.3 [M+H]⁺.

### Preparation of 3-(7-(4-((3-oxa-7,9-diazabicyclo[3.3.1]nonan-7-yl)methyl)piperidin-1-yl)-1-methyl-1H-indazol-3-yl)piperidine-2,6-dione

A solution of tert-butyl 7-((1-(3-(2,6-dioxopiperidin-3-yl)-1-methyl-1H-indazol-7-yl)piperidin-4-yl)methyl)-3-oxa-7,9-diazabicyclo[3.3.1]nonane-9-carboxylate (303.2 mg, 535.0 μmol) in DCM (3 mL) was treated with trifluoroacetic acid (412 μL, 5.35 mmol). The mixture was stirred at room temperature overnight. The reaction mixture was concentrated under reduced pressure to give 3-(7-(4-((3-oxa-7,9-diazabicyclo[3.3.1]nonan-7-yl)methyl)piperidin-1-yl)-1-methyl-1H-indazol-3-yl)piperidine-2,6-dione (310 mg) as a purplish oil, which was carried forward without purification. LC-MS (ESI): m/z=467.4 [M+H]⁺.

### Preparation of 2-((6-((5-chloro-2-(7-((1-(3-(2,6-dioxopiperidin-3-yl)-1-methyl-1H-indazol-7-yl)piperidin-4-yl)methyl)-3-oxa-7,9-diazabicyclo[3.3.1]nonan-9-yl)pyrimidin-4-yl)amino)-1-isopropyl-2-oxo-1,2-dihydro-1,8-naphthyridin-3-yl)oxy)-N-methylacetamide

A mixture of 2-((6-((2,5-dichloropyrimidin-4-yl)amino)-1-isopropyl-2-oxo-1,2-dihydro-1,8-naphthyridin-3-yl)oxy)-N-methylacetamide (113 mg, 258 μmol), 3-(7-(4-((3-oxa-7,9-diazabicyclo[3.3.1]nonan-7-yl)methyl)piperidin-1-yl)-1-methyl-1H-indazol-3-yl)piperidine-2,6-dione, TFA (150 mg, 258 μmol), and DIPEA (225 μL, 1.29 mmol) in DMSO (2 mL) was stirred at 100° C. in a heated reaction block overnight. The reaction mixture was filtered through a 0.45 μm hydrophilic PTFE syringe filter, rinsing with DMSO. The filtrate was purified by preparative HPLC to afford 2-((6-((5-chloro-2-(7-((1-(3-(2,6-dioxopiperidin-3-yl)-1-methyl-1H-indazol-7-yl)piperidin-4-yl)methyl)-3-oxa-7,9-diazabicyclo[3.3.1]nonan-9-yl)pyrimidin-4-yl)amino)-1-isopropyl-2-oxo-1,2-dihydro-1,8-naphthyridin-3-yl)oxy)-N-methylacetamide (13.7 mg) was obtained as an off-white solid. ¹H NMR (400 MHz, DMSO-d₆) δ (ppm)=10.87 (s, 1H), 9.15 (s, 1H), 8.61 (d, J=2.5 Hz, 1H), 8.34-8.25 (m, 1H), 8.13 (s, 1H), 7.96 (d, J=4.7 Hz, 1H), 7.36 (dd, J=6.1, 2.8 Hz, 1H), 7.00 (d, J=4.3 Hz, 2.6H), 6.61 (s, 0.4H), 5.95 (s, 1H), 4.56 (m, 3H), 4.33 (dd, J=9.5, 5.1 Hz, 1H), 4.24 (m, 4H), 3.84 (d, J=11.0 Hz, 2H), 3.69 (t, J=11.0 Hz, 2H), 3.21 (s, 1H), 2.95 (d, J=10.8 Hz, 2H), 2.73-2.59 (m, 4H), 2.54 (s, 1H), 2.31 (dd, J=10.0, 4.9 Hz, 3H), 2.21-2.04 (m, 3H), 1.87 (d, J=12.4 Hz, 2H), 1.70 (s, 1H), 1.56 (d, J=6.9 Hz, 6H), 1.35 (d, J=12.9 Hz, 2H). LC-MS (ESI): m/z=867.5 [M+H]⁺.

### Example 154: 2-((6-((5-chloro-2-(4-((4-(3-(2,6-dioxopiperidin-3-yl)-1-methyl-1H-indazol-6-yl)piperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide (Compound 214)

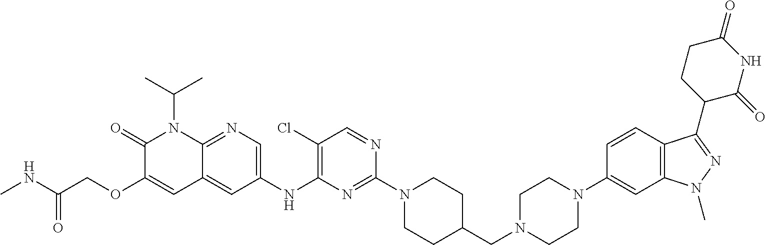 [SMILES: CNC(=O)COc1cc2cc(Nc3nc(N4CCC(CN5CCN(c6ccc7c(C8CCC(=O)NC8=O)nn(C)c7c6)CC5)CC4)ncc3Cl)cnc2n(C(C)C)c1=O]

### Preparation of tert-butyl 4-(3-(2,6-bis(benzyloxy)pyridin-3-yl)-1-methyl-1H-indazol-6-yl)piperazine-1-carboxylate

A mixture of 3-(2,6-bis(benzyloxy)pyridin-3-yl)-6-bromo-1-methyl-1H-indazole (50.0 g, 99.9 mmol, 1.00 equiv.), tert-butyl piperazine-1-carboxylate (37.2 g, 200 mmol, 2.00 equiv.), Pd(OAc) 2 (3.45 g, 0.100 equiv.), BINAP (2.80 g, 0.100 eq) and Cs₂CO₃ (96.7 g, 297 mmol, 3.00 equiv.) in 1,4-dioxane (500 mL) was degassed and purged with N₂ three times. The mixture was stirred at 100° C. for 1 h under N₂ atmosphere. To the reaction mixture was added H₂O (200 mL) and filtered. The aqueous phase was separated and extracted with ethyl acetate (300 mL×2). The organic phases were combined and washed with brine (400) mL, dried over Na₂SO₄, filtered, and concentrated under reduced pressure to give a residue. The residue was purified by column chromatography (petroleum ether/ethyl acetate=10/1 to 5/1) to afford tert-butyl 4-(3-(2,6-bis(benzyloxy)pyridin-3-yl)-1-methyl-1H-indazol-6-yl)piperazine-1-carboxylate (65.0 g) as a yellow solid.

LC-MS (ESI): m/z=606.4 [M+H]⁺

### Preparation of tert-butyl 4-(3-(2,6-dioxopiperidin-3-yl)-1-methyl-1H-indazol-6-yl)piperazine-1-carboxylate

A mixture of tert-butyl 4-(3-(2,6-bis(benzyloxy)pyridin-3-yl)-1-methyl-1H-indazol-6-yl)piperazine-1-carboxylate (65.0 g, 107 mmol, 1.00 equiv.) in EtOH (650 mL), THF (600 mL), and AcOH (65.0 mL) was added Pd/C (65.0 g, 10% purity) and Pd(OH)₂ (65.0 g, 20% purity) under N₂ atmosphere. The suspension was degassed and purged with H₂ three times. The mixture was stirred under H₂ (50 psi) at 50° C. for 4 hrs. The reaction mixture was filtered, washed with THF (5.00 L), and concentrated under reduced pressure to give a residue. The residue was triturated with petroleum ether (50.0 mL) and ethyl acetate (50.0 mL) to afford tert-butyl 4-(3-(2,6-dioxopiperidin-3-yl)-1-methyl-1H-indazol-6-yl)piperazine-1-carboxylate (18.4 g) as a pink solid.

LC-MS (ESI): m/z=428.3 [M+H]⁺

### Preparation of 3-(1-methyl-6-(piperazin-1-yl)-1H-indazol-3-yl)piperidine-2,6-dione

A solution of tert-butyl 4-(3-(2,6-dioxopiperidin-3-yl)-1-methyl-1H-indazol-6-yl)piperazine-1-carboxylate (14.0 g, 32.8 mmol, 1.00 equiv.) in HCl/ethyl acetate (100 mL) was stirred at 20° C. for 2 h. The reaction mixture was filtered and concentrated under reduced pressure to give 3-(1-methyl-6-(piperazin-1-yl)-1H-indazol-3-yl)piperidine-2,6-dione (11.5 g) as a light yellow solid.

LC-MS (ESI): m/z=328.4 [M+H]⁺

### Preparation of 2-((6-((2-(4-(1,3-dioxolan-2-yl)piperidin-1-yl)-5-chloropyrimidin-4-yl)amino)-1-isopropyl-2-oxo-1,2-dihydro-1,8-naphthyridin-3-yl)oxy)-N-methylacetamide

A mixture of 2-((6-((2,5-dichloropyrimidin-4-yl)amino)-1-isopropyl-2-oxo-1,2-dihydro-1,8-naphthyridin-3-yl)oxy)-N-methylacetamide (500 mg, 1.14 mmol), 4-(1,3-dioxolan-2-yl)piperidine (216 mg, 1.37 mmol), and DIPEA (598 μL, 3.43 mmol) in DMSO (5.5 mL) was stirred at 80° C. in a heated reaction block overnight. The reaction mixture was diluted with brine. The resulting suspension was filtered. The precipitate was washed with a small amount of MeOH/water, then collected and dried under vacuum to afford 2-((6-((2-(4-(1,3-dioxolan-2-yl)piperidin-1-yl)-5-chloropyrimidin-4-yl)amino)-1-isopropyl-2-oxo-1,2-dihydro-1,8-naphthyridin-3-yl)oxy)-N-methylacetamide (393.7 mg) as a tan solid and was carried forward without additional purification. LC-MS (ESI): m/z=558.2 [M+H]⁺.

### Preparation of 2-((6-((5-chloro-2-(4-formylpiperidin-1-yl)pyrimidin-4-yl)amino)-1-isopropyl-2-oxo-1,2-dihydro-1,8-naphthyridin-3-yl)oxy)-N-methylacetamide

A solution of 2-((6-((2-(4-(1,3-dioxolan-2-yl)piperidin-1-yl)-5-chloropyrimidin-4-yl)amino)-1-isopropyl-2-oxo-1,2-dihydro-1,8-naphthyridin-3-yl)oxy)-N-methylacetamide (393.7 mg, 705.5 μmol) in formic acid (2.662 mL, 70.55 mmol) was stirred at 80° C. for 3 h. The reaction mixture was concentrated under reduced pressure to give 2-((6-((5-chloro-2-(4-formylpiperidin-1-yl)pyrimidin-4-yl)amino)-1-isopropyl-2-oxo-1,2-dihydro-1,8-naphthyridin-3-yl)oxy)-N-methylacetamide (395 mg, 705 μmol) and carried forward without purification. LC-MS (ESI): m/z=514.3 [M+H]⁺.

### Preparation of 2-((6-((5-chloro-2-(4-((4-(3-(2,6-dioxopiperidin-3-yl)-1-methyl-1H-indazol-6-yl)piperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide

A mixture of crude 2-((6-((5-chloro-2-(4-formylpiperidin-1-yl)pyrimidin-4-yl)amino)-1-isopropyl-2-oxo-1,2-dihydro-1,8-naphthyridin-3-yl)oxy)-N-methylacetamide, formic acid (131 mg, 234 μmol), 3-(1-methyl-6-(piperazin-1-yl)-1H-indazol-3-yl)piperidine-2,6-dione, HCl (128 mg, 351 μmol), and potassium acetate (68.9 mg, 702 μmol) in THF (2.0 mL) and DMF (0.50 mL) was treated with sodium triacetoxyborohydride (74.4 mg, 351 μmol). The reaction mixture was stirred at room temperature overnight. The reaction mixture was concentrated under reduced pressure. The residue was taken up in DMSO/water and filtered through a 0.45 μm hydrophilic PTFE syringe filter. The filtrate was purified by preparative HPLC to give 2-((6-((5-chloro-2-(4-((4-(3-(2,6-dioxopiperidin-3-yl)-1-methyl-1H-indazol-6-yl)piperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide (104.1 mg) as a white solid. ¹H NMR (400 MHz, CD₃SOCD₃, 300 K) δ (ppm)=10.77 (s, 1H), 8.96 (s, 1H), 8.62 (d, J=2.5 Hz, 1H), 8.22 (d, J=2.4 Hz, 1H), 8.00 (s, 1H), 7.86 (br d, J=4.6 Hz, 1H), 7.42 (d, J=8.9 Hz, 1H), 7.02 (s, 1H), 6.84 (dd, J=1.5, 9.0 Hz, 1H), 6.76 (s, 1H), 5.98-5.79 (m, 1H), 4.50 (s, 2H), 4.39 (br d, J=11.6 Hz, 2H), 4.19 (dd, J=5.1, 9.1 Hz, 1H), 3.82 (s, 3H), 3.14 (br s, 4H), 2.79 (br t, J=11.9 Hz, 2H), 2.60 (d, J=4.6 Hz, 3H), 2.57-2.51 (m, 2H), 2.30-2.02 (m, 5H), 1.82-1.63 (m, 3H), 1.49 (d, J=6.9 Hz, 6H), 1.06-0.90 (m, 2H). LC-MS (ESI): m/z=825.4 [M+H]⁺.

### Example 155: 2-((6-((5-chloro-2-(4-(((2R)-4-(3-(2,6-dioxopiperidin-3-yl)-1-methyl-1H-indazol-6-yl)-2-methylpiperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide (Compound 215a)

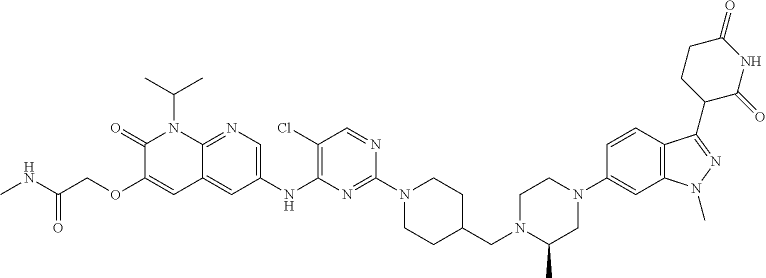 [SMILES: CNC(=O)COc1cc2cc(Nc3nc(N4CCC(CN5CCN(c6ccc7c(C8CCC(=O)NC8=O)nn(C)c7c6)C[C@H]5C)CC4)ncc3Cl)cnc2n(C(C)C)c1=O]

### Preparation of tert-butyl (R)-4-(3-(2,6-bis(benzyloxy)pyridin-3-yl)-1-methyl-1H-indazol-6-yl)-2-methylpiperazine-1-carboxylate

To a solution of 3-(2,6-bis(benzyloxy)pyridin-3-yl)-6-bromo-1-methyl-1H-indazole (15.0 g, 29.9 mmol, 1.00 equiv.) and tert-butyl (R)-2-methylpiperazine-1-carboxylate (12.0 g, 59.9 mmol, 2.00 equiv.) in dioxane (150 mL) was added t-BuONa (8.64 g, 89.9 mmol, 3.00 equiv.) and RuPhos (1.40 g, 3.00 mmol, 0.10 equiv.). The mixture was degassed and purged with N₂ three times. To this solution was added Pd₂(dba)₃ (2.75 g, 3.00 mmol, 0.10 equiv.). The mixture was stirred at 100° C. for 1 h. The reaction mixture was diluted with H₂O (200 mL) and extracted with ethyl acetate (100 mL×3). The combined organic layers were washed with brine (100 mL×2), dried over Na₂SO₄, filtered, and concentrated under reduced pressure to give a residue. The residue was purified by column chromatography (SiO₂, petroleum ether:ethyl acetate=5:1 to 1:1) to give tert-butyl (R)-4-(3-(2,6-bis(benzyloxy)pyridin-3-yl)-1-methyl-1H-indazol-6-yl)-2-methylpiperazine-1-carboxylate (13.2 g, 21.3 mmol) as a yellow solid.

LC-MS (ESI): m/z=621.0 (M+H)+

¹H NMR: (400 MHz, DMSO-d₆) § 7.89 (br d, J=8.0 Hz, 1H), 7.54-7.23 (m, 10H), 6.88-6.71 (m, 2H), 6.55 (d, J=8.1 Hz, 1H), 5.80-5.71 (m, 1H), 5.42 (br d, J=18.8 Hz, 4H), 4.29-4.18 (m, 1H), 4.02-3.92 (m, 3H), 3.90-3.77 (m, 1H), 3.69-3.60 (m, 1H), 3.60-3.50 (m, 1H), 3.20 (br t, J=10.9 Hz, 1H), 2.93-2.83 (m, 1H), 2.74-2.62 (m, 1H), 1.43 (s, 9H), 1.22 (br d, J=6.6 Hz, 3H)

### Preparation of tert-butyl(2R)-4-(3-(2,6-dioxopiperidin-3-yl)-1-methyl-1H-indazol-6-yl)-2-methylpiperazine-1-carboxylate

To a solution of tert-butyl (R)-4-(3-(2,6-bis(benzyloxy)pyridin-3-yl)-1-methyl-1H-indazol-6-yl)-2-methylpiperazine-1-carboxylate (12.0 g, 19.3 mmol, 1.00 equiv.) in THF (120 mL), EtOH (120 mL), and AcOH (12 mL) was added Pd/C (3.00 g, 2.82 mmol, 10.0% purity) and Pd(OH)₂ (3.00 g, 4.27 mmol, 20.0% purity) under N₂ atmosphere. The suspension was degassed and purged with H₂ three times. The mixture was stirred under H₂ (50 Psi) at 50° C. for 4 h. The reaction mixture was filtered, washed with THF (200 ml), and concentrated under reduced pressure to give tert-butyl(2R)-4-(3-(2,6-dioxopiperidin-3-yl)-1-methyl-1H-indazol-6-yl)-2-methylpiperazine-1-carboxylate (9.5 g, crude) as a brown solid.

LC-MS (ESI): m/z=442.4 (M+H)+

### Preparation of 3-(1-methyl-6-((R)-3-methylpiperazin-1-yl)-1H-indazol-3-yl)piperidine-2,6-dione

A mixture of tert-butyl(2R)-4-(3-(2,6-dioxopiperidin-3-yl)-1-methyl-1H-indazol-6-yl)-2-methylpiperazine-1-carboxylate (6.50 g, 14.7 mmol, 1.00 equiv.) in HCl/dioxane (70.0 mL) was stirred at 25° C. for 1 h. The reaction mixture was filtered and concentrated under reduced pressure to give 3-(1-methyl-6-((R)-3-methylpiperazin-1-yl)-1H-indazol-3-yl)piperidine-2,6-dione (5.00 g, 14.3 mmol) as an off-white solid.

LC-MS (ESI): m/z=342.4 (M+H)⁺

¹H NMR: (400 MHz, DMSO-d₆) § 10.85 (s, 1H), 9.66 (br d, J=9.8 Hz, 1H), 9.50-9.33 (m, 1H), 7.56 (d, J=8.9 Hz, 1H), 7.02-6.89 (m, 2H), 5.82-5.52 (m, 3H), 4.28 (dd, J=5.1, 9.3 Hz, 1H), 3.92 (s, 3H), 3.89-3.78 (m, 2H), 3.44-3.30 (m, 2H), 3.20-3.04 (m, 2H), 2.88 (dd, J=10.8, 12.8 Hz, 1H), 2.71-2.55 (m, 2H), 2.39-2.24 (m, 1H), 2.21-2.09 (m, 1H), 1.34 (d, J=6.5 Hz, 3H)

### Preparation of 2-((6-((5-chloro-2-(4-(((2R)-4-(3-(2,6-dioxopiperidin-3-yl)-1-methyl-1H-indazol-6-yl)-2-methylpiperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide

A mixture of crude 2-((6-((5-chloro-2-(4-formylpiperidin-1-yl)pyrimidin-4-yl)amino)-1-isopropyl-2-oxo-1,2-dihydro-1,8-naphthyridin-3-yl)oxy)-N-methylacetamide (131 mg, 234 μmol), 3-(1-methyl-6-((R)-3-methylpiperazin-1-yl)-1H-indazol-3-yl)piperidine-2,6-dione, HCl (133 mg, 351 μmol), and potassium acetate (68.9 mg, 702 μmol) in THF (2.0 mL) and DMF (0.50 mL) was treated with sodium triacetoxyborohydride (74.4 mg, 351 μmol). The reaction mixture was stirred at room temperature overnight. The reaction mixture was concentrated under reduced pressure. The residue was taken up in DMSO/water and filtered through a 0.45 μm hydrophilic PTFE syringe filter. The filtrate was purified by preparative HPLC to give 2-((6-((5-chloro-2-(4-(((2R)-4-(3-(2,6-dioxopiperidin-3-yl)-1-methyl-1H-indazol-6-yl)-2-methylpiperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide (122.7 mg) as a white solid. ¹H NMR (400 MHz, DMSO-d₆) δ (ppm)=10.77 (s, 1H), 8.95 (s, 1H), 8.62 (d, J=2.5 Hz, 1H), 8.23 (d, J=2.5 Hz, 1H), 8.00 (s, 1H), 7.86 (br d, J=4.5 Hz, 1H), 7.42 (d, J=8.9 Hz, 1H), 7.01 (s, 1H), 6.84 (dd, J=1.3, 9.0 Hz, 1H), 6.75 (s, 1H), 5.88 (br s, 1H), 4.50 (s, 2H), 4.39 (br d, J=12.1 Hz, 2H), 4.18 (dd, J=5.1, 9.1 Hz, 1H), 3.82 (s, 3H), 3.43 (br d, J=8.5 Hz, 2H), 2.95-2.71 (m, 4H), 2.60 (d, J=4.6 Hz, 3H), 2.57-2.49 (m, 3H), 2.30-2.17 (m, 2H), 2.14-2.03 (m, 1H), 1.92 (br d, J=9.3 Hz, 1H), 1.84-1.66 (m, 2H), 1.61 (br d, J=12.5 Hz, 1H), 1.50 (d, J=6.9 Hz, 6H), 1.09-0.84 (m, 5H). LC-MS (ESI): m/z=839.4 [M+H]⁺.

### Example 156: 2-((6-((5-chloro-2-(4-(((2S)-4-(3-(2,6-dioxopiperidin-3-yl)-1-methyl-1H-indazol-6-yl)-2-methylpiperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide

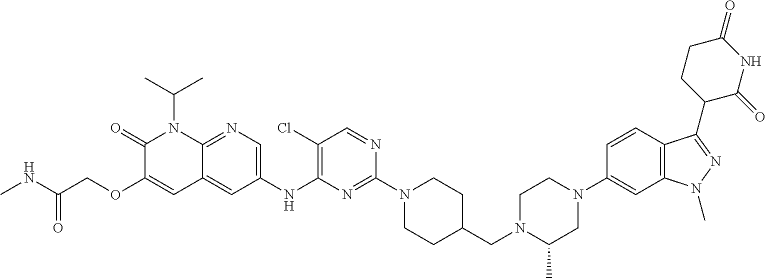 [SMILES: CNC(=O)COc1cc2cc(Nc3nc(N4CCC(CN5CCN(c6ccc7c(C8CCC(=O)NC8=O)nn(C)c7c6)C[C@@H]5C)CC4)ncc3Cl)cnc2n(C(C)C)c1=O]

### Preparation of tert-butyl(S)-4-(3-(2,6-bis(benzyloxy)pyridin-3-yl)-1-methyl-1H-indazol-6-yl)-2-methylpiperazine-1-carboxylate

A mixture of 3-(2,6-bis(benzyloxy)pyridin-3-yl)-6-bromo-1-methyl-1H-indazole (15.0 g, 29.9 mmol, 1.00 equiv.), tert-butyl(S)-2-methylpiperazine-1-carboxylate (12.0 g, 59.9 mmol, 2.00 equiv., Pd₂(dba)₃ (2.75 g, 3.00 mmol, 0.10 equiv.), RuPhos (1.40 g, 3.00 mmol, 0.10 equiv.) and t-BuONa (8.64 g, 89.9 mmol, 3.00 equiv.) in dioxane (150 mL) was degassed and purged with N₂ three times. The mixture was stirred at 100° C. for 1 h under N₂ atmosphere. The reaction mixture was quenched by the addition of H₂O (40.0 mL) at 25° C., diluted with ethyl acetate (100 mL), and extracted with ethyl acetate (100 mL×2). The combined organic layers were washed with brine (100 mL×3), dried over anhydrous Na₂SO₄, filtered, and concentrated under reduced pressure to give a residue. The residue was purified by column chromatography (petroleum ether:ethyl acetate=5:1 to 1:1) to give tert-butyl(S)-4-(3-(2,6-bis(benzyloxy)pyridin-3-yl)-1-methyl-1H-indazol-6-yl)-2-methylpiperazine-1-carboxylate (18.5 g, 25.3 mmol) as a yellow oil.

LC-MS (ESI): m/z=621.2 (M+H)+

¹H NMR: (400 MHz, DMSO-d₆) § 7.91-7.85 (m, 1H), 7.53-7.52 (m, 1H), 7.52 (d, J=9.0 Hz, 1H), 7.48-7.43 (m, 2H), 7.42-7.34 (m, 5H), 7.33-7.25 (m, 4H), 6.84 (s, 1H), 6.79 (dd, J=1.6, 9.1 Hz, 1H), 6.55 (d, J=8.0 Hz, 1H), 5.42 (d, J=18.6 Hz, 4H), 4.22 (br d, J=2.5 Hz, 1H), 3.96 (s, 3H), 3.83 (br d, J=13.1 Hz, 1H), 3.69-3.52 (m, 2H), 3.26-3.15 (m, 1H), 2.96-2.84 (m, 1H), 2.75-2.64 (m, 1H), 1.43 (s, 10H), 1.22 (d, J=6.6 Hz, 3H)

### Preparation of tert-butyl(2S)-4-(3-(2,6-dioxopiperidin-3-yl)-1-methyl-1H-indazol-6-yl)-2-methylpiperazine-1-carboxylate

To a solution of tert-butyl(S)-4-(3-(2,6-bis(benzyloxy)pyridin-3-yl)-1-methyl-1H-indazol-6-yl)-2-methylpiperazine-1-carboxylate (15.0 g, 24.2 mmol, 1.00 equiv.) in EtOH (150 mL) and THF (150 mL) was added Pd/C (3.70 g, 3.48 mmol, 10.0% purity), Pd(OH)₂ (3.70 g, 26.3 mmol, 1.09 equiv.), and AcOH (15.0 mL) under N₂. The suspension was degassed under vacuum and purged with H₂ three times. The mixture was stirred under H₂ (50 psi) at 50° C. for 4 h. The reaction mixture was filtered, washed with THF (200 mL), and concentrated under reduced pressure to give tert-butyl(2S)-4-(3-(2,6-dioxopiperidin-3-yl)-1-methyl-1H-indazol-6-yl)-2-methylpiperazine-1-carboxylate (9.00 g, 16.9 mmol) as a yellow solid.

LC-MS (ESI): m/z=442.4 (M+H)+

### Preparation of 3-(1-methyl-6-((S)-3-methylpiperazin-1-yl)-1H-indazol-3-yl)piperidine-2,6-dione

A mixture of tert-butyl(2S)-4-(3-(2,6-dioxopiperidin-3-yl)-1-methyl-1H-indazol-6-yl)-2-methylpiperazine-1-carboxylate (9.00 g, 20.3 mmol, 1.00 eq) in HCl/dioxane (90.0 mL) was stirred at 25° C. for 1 h. The reaction mixture was filtered and concentrated under reduced pressure to give 3-(1-methyl-6-((S)-3-methylpiperazin-1-yl)-1H-indazol-3-yl)piperidine-2,6-dione (5.50 g, 14.3 mmol) as a yellow solid.

LC-MS (ESI): m/z=342.1 (M+H)⁺

¹H NMR: (400 MHz, DMSO-d₆)

δ 10.86 (s, 1H), 9.75-9.39 (m, 2H), 7.56 (d, J=8.9 Hz, 1H), 7.02-6.93 (m, 2H), 4.28 (br dd, J=5.0, 9.4 Hz, 1H), 3.92 (s, 3H), 3.89-3.79 (m, 2H), 3.46-3.29 (m, 2H), 3.12 (br d, J=8.6 Hz, 2H), 2.90 (br dd, J=10.9, 12.6 Hz, 1H), 2.70-2.55 (m, 3H), 2.38-2.26 (m, 1H), 2.22-2.09 (m, 1H), 1.35 (d, J=6.5 Hz, 3H)

### Preparation of 2-((6-((5-chloro-2-(4-(((2S)-4-(3-(2,6-dioxopiperidin-3-yl)-1-methyl-1H-indazol-6-yl)-2-methylpiperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide

A mixture of crude 2-((6-((5-chloro-2-(4-formylpiperidin-1-yl)pyrimidin-4-yl)amino)-1-isopropyl-2-oxo-1,2-dihydro-1,8-naphthyridin-3-yl)oxy)-N-methylacetamide (131 mg, 234 μmol), 3-(1-methyl-6-((S)-3-methylpiperazin-1-yl)-1H-indazol-3-yl)piperidine-2,6-dione (120 mg, 351 μmol), and potassium acetate (68.9 mg, 702 μmol) in THF (2.0 mL) in DMF (0.50 mL) was treated with sodium triacetoxyborohydride (74.4 mg, 351 μmol). The reaction mixture was stirred at room temperature overnight. The reaction mixture was concentrated under reduced pressure. The residue was taken up in DMSO/water and filtered through a 0.45 μm hydrophilic PTFE syringe filter. The filtrate was purified by preparative HPLC to give 2-((6-((5-chloro-2-(4-(((2S)-4-(3-(2,6-dioxopiperidin-3-yl)-1-methyl-1H-indazol-6-yl)-2-methylpiperazin-1-yl)methyl)piperidin-1-yl)pyrimidin-4-yl)amino)-1-isopropyl-2-oxo-1,2-dihydro-1,8-naphthyridin-3-yl)oxy)-N-methylacetamide (111.8 mg) as a white solid. ¹H NMR (400 MHz, CD₃SOCD₃, 300 K) δ (ppm)=10.77 (s, 1H), 8.95 (s, 1H), 8.62 (d, J=2.5 Hz, 1H), 8.23 (d, J=2.5 Hz, 1H), 8.00 (s, 1H), 7.86 (br d, J=4.8 Hz, 1H), 7.42 (d, J=9.0 Hz, 1H), 7.01 (s, 1H), 6.84 (dd, J=1.4, 9.1 Hz, 1H), 6.75 (s, 1H), 5.88 (br s, 1H), 4.50 (s, 2H), 4.39 (br d, J=12.0 Hz, 2H), 4.18 (dd, J=5.1, 9.1 Hz, 1H), 3.82 (s, 3H), 3.43 (br d, J=8.3 Hz, 2H), 2.94-2.72 (m, 4H), 2.60 (d, J=4.6 Hz, 3H), 2.57-2.46 (m, 4H), 2.31-2.18 (m, 2H), 2.14-2.02 (m, 1H), 1.92 (br dd, J=4.6, 12.5 Hz, 1H), 1.82-1.67 (m, 2H), 1.61 (br d, J=11.6 Hz, 1H), 1.50 (d, J=7.0 Hz, 6H), 1.08-0.84 (m, 5H). LC-MS (ESI): m/z=839.4 [M+H]⁺

### Example 157. 2-((6-((5-Chloro-2-((3S)-4-(((3S)-1-(3-(2,6-dioxopiperidin-3-yl)-1-methyl-1H-indazol-6-yl)pyrrolidin-3-yl)methyl)-3-methylpiperazin-1-yl)pyrimidin-4-yl)amino)-1-isopropyl-2-oxo-1,2-dihydro-1,8-naphthyridin-3-yl)oxy)-N-methylacetamide (Compound 216b)

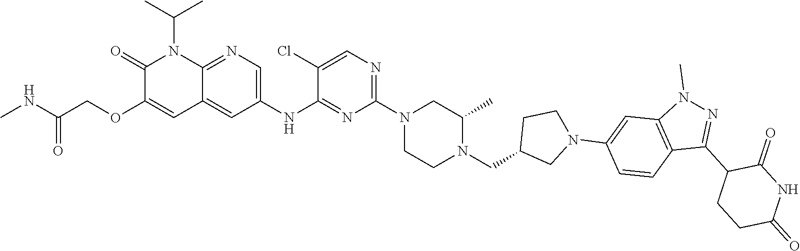 [SMILES: CNC(=O)COc1cc2cc(Nc3nc(N4CCN(C[C@@H]5CCN(c6ccc7c(C8CCC(=O)NC8=O)nn(C)c7c6)C5)[C@@H](C)C4)ncc3Cl)cnc2n(C(C)C)c1=O]

### Preparation of (R)-(1-(3-(2,6-bis(benzyloxy)pyridin-3-yl)-1-methyl-1H-indazol-6-yl)pyrrolidin-3-yl)methanol

To a solution of 3-(2,6-bis(benzyloxy)pyridin-3-yl)-6-bromo-1-methyl-1H-indazole (6 g, 11.99 mmol, 1 equiv.), (R)-pyrrolidin-3-ylmethanol (1.82 g, 17.99 mmol, 1.5 equiv.) and NaOtBu (2.30 g, 23.98 mmol, 2 equiv.) in dioxane (100 mL) was added Pd₂(dba)₃ (1.10 g, 1.20 mmol, 0.1 equiv.) and rac-BINAP (1.49 g, 2.40 mmol, 0.2 equiv.). The mixture was stirred at 100° C. under a nitrogen atmosphere for 5 hours. The reaction was diluted with water (100 mL) and extracted with ethyl acetate (200 mL×2). The combined organic layers were washed with brine 100 mL×1), dried over Na₂SO₄, filtered, and concentrated under reduced pressure to give a residue which was purified by silica gel column chromatography (petroleum ether/ethyl acetate, 5:1 to 1:1) to give (R)-(1-(3-(2,6-bis(benzyloxy)pyridin-3-yl)-1-methyl-1H-indazol-6-yl)pyrrolidin-3-yl)methanol (2.9 g, 5.05 mmol) as a yellow oil. LC-MS (ESI): m/z=521.2 [M+H]⁺.

### Preparation of 3-(6-((R)-3-(hydroxymethyl)pyrrolidin-1-yl)-1-methyl-1H-indazol-3-yl)piperidine-2,6-dione

To a solution of (R)-(1-(3-(2,6-bis(benzyloxy)pyridin-3-yl)-1-methyl-1H-indazol-6-yl)pyrrolidin-3-yl)methanol (2.9 g, 5.57 mmol, 1 equiv.) in THF (20 mL) was added Pd(OH)₂ (1.56 g, 2.23 mmol, 20% by weight, 0.4 equiv.) and Pd/C (2.37 g, 2.23 mmol, 10% by weight, 0.4 equiv.). The suspension was degassed and purged with hydrogen three times and then stirred under a hydrogen atmosphere (balloon, 15 psi) at 25° C. for 12 hours. The reaction mixture was filtered through a bed of celite and rinsed with THE. The resulting filtrate was concentrated under reduced pressure to give 3-(6-((R)-3-(hydroxymethyl)pyrrolidin-1-yl)-1-methyl-1H-indazol-3-yl)piperidine-2,6-dione (1.9 g, 5.55 mmol) as a yellow oil. LC-MS (ESI): m/z=343.2 [M+H]⁺.

### Preparation of 3-(6-((R)-3-(chloromethyl)pyrrolidin-1-yl)-1-methyl-1H-indazol-3-yl)piperidine-2,6-dione

To a solution of 3-(6-((R)-3-(hydroxymethyl)pyrrolidin-1-yl)-1-methyl-1H-indazol-3-yl)piperidine-2,6-dione (1.9 g, 5.55 mmol, 1 equiv.) in DCM (20 mL) was added PPh₃ (1.46 g, 5.55 mmol, 1 equiv.) and CC14 (4.27 g, 27.75 mmol, 2.68 mL, 5 equiv.). The mixture was stirred at 25° C. for 12 hours. The reaction was diluted with water (100 mL) and extracted with ethyl acetate (200 mL×2). The combined organic layers were washed with brine (100 mL×1), dried over anhydrous sodium sulfate, filtered, and concentrated under reduced pressure to give a residue that was purified by silica gel column chromatography (petroleum ether/ethyl acetate, 3:1 to 1:1) to give 3-(6-((R)-3-(chloromethyl)pyrrolidin-1-yl)-1-methyl-1H-indazol-3-yl)piperidine-2,6-dione as a purple solid that was used without further purification assuming theoretical yield. LC-MS (ESI): m/z=361.0 [M+H]⁺. ¹H NMR (400 MHz, DMSO-d₆) ¿=10.83 (s, 1H), 7.47 (d, J=8.8 Hz, 1H), 6.56 (d, J=8.8 Hz, 1H), 6.37 (s, 1H), 4.26-4.20 (m, 1H), 3.85 (s, 3H), 3.79-3.74 (m, 2H), 3.54-3.49 (m, 1H), 3.45-3.39 (m, 2H), 3.17 (t, J=3.6 Hz, 1H), 2.76-2.71 (m, 1H), 2.60 (d, J=5.6 Hz, 2H), 2.26 (d, J=5.6 Hz, 1H), 2.20-2.15 (m, 2H), 1.88-1.82 (m, 1H).

### Preparation of tert-butyl (3S)-4-(((3S)-1-(3-(2,6-dioxopiperidin-3-yl)-1-methyl-1H-indazol-6-yl)pyrrolidin-3-yl)methyl)-3-methylpiperazine-1-carboxylate

To a solution of 3-(6-((R)-3-(chloromethyl)pyrrolidin-1-yl)-1-methyl-1H-indazol-3-yl)piperidine-2,6-dione (200 mg, 1 equiv., 554 mmol) and tert-butyl(S)-3-methylpiperazine-1-carboxylate (167 mg, 1.5 equiv., 831 μmol) in DMF (1 mL) was added sodium iodide (83.1 mg, 1 equiv., 554 mmol) and DIEA (287 mg, 386 mL, 4 equiv., 2.22 mmol). The mixture was stirred at 100° C. for 16 h. The mixture was purified by preparative RP-HPLC to afford tert-butyl (3S)-4-(((3S)-1-(3-(2,6-dioxopiperidin-3-yl)-1-methyl-1H-indazol-6-yl)pyrrolidin-3-yl)methyl)-3-methylpiperazine-1-carboxylate (179 mg, 341 μmol). LC-MS (ESI): m/z 525.4 [M+H]⁺.

### Preparation of 3-(1-methyl-6-((S)-3-(((S)-2-methylpiperazin-1-yl)methyl)pyrrolidin-1-yl)-1H-indazol-3-yl)piperidine-2,6-dione dihydrochloride

To a solution of tert-butyl (3S)-4-(((3S)-1-(3-(2,6-dioxopiperidin-3-yl)-1-methyl-1H-indazol-6-yl)pyrrolidin-3-yl)methyl)-3-methylpiperazine-1-carboxylate (179 mg, 1 equiv., 341 μmol) in DCM (5 mL) was added HCl in dioxane (373 mg, 2.56 mL, 4.0 molar, 30 equiv., 10.2 mmol). The mixture was stirred for 16 h at ambient temperature. The mixture was concentrated to dryness to afford 3-(1-methyl-6-((S)-3-(((S)-2-methylpiperazin-1-yl)methyl)pyrrolidin-1-yl)-1H-indazol-3-yl)piperidine-2,6-dione dihydrochloride (188 mg, 378 μmol) that was used without further purification. LC-MS (ESI): m/z 425.3 [M+H]⁺.

### Preparation of 2-((6-((5-chloro-2-((3S)-4-(((3S)-1-(3-(2,6-dioxopiperidin-3-yl)-1-methyl-1H-indazol-6-yl)pyrrolidin-3-yl)methyl)-3-methylpiperazin-1-yl)pyrimidin-4-yl)amino)-1-isopropyl-2-oxo-1,2-dihydro-1,8-naphthyridin-3-yl)oxy)-N-methylacetamide

A mixture of 2-((6-((2,5-dichloropyrimidin-4-yl)amino)-1-isopropyl-2-oxo-1,2-dihydro-1,8-naphthyridin-3-yl)oxy)-N-methylacetamide (53 mg, 1.2 equiv., 0.12 mmol), 3-(1-methyl-6-((S)-3-(((S)-2-methylpiperazin-1-yl)methyl)pyrrolidin-1-yl)-1H-indazol-3-yl)piperidine-2,6-dione dihydrochloride (50 mg, 1 equiv., 0.10 mmol), and CsF (76 mg, 5.0 equiv., 0.50 mmol) in DMSO was treated with DIEA (78 mg, 0.11 mL, 6.00 equiv., 0.60 mmol). The reaction mixture was then stirred at 80° C. for 15 h. The reaction was purified directly after cooling by preparative RP-HPLC to afford 2-((6-((5-chloro-2-((3S)-4-(((3S)-1-(3-(2,6-dioxopiperidin-3-yl)-1-methyl-1H-indazol-6-yl)pyrrolidin-3-yl)methyl)-3-methylpiperazin-1-yl)pyrimidin-4-yl)amino)-1-isopropyl-2-oxo-1,2-dihydro-1,8-naphthyridin-3-yl)oxy)-N-methylacetamide (45 mg, 55 μmol). LC-MS (ESI): m/z 825.4 [M+H]⁺. ¹H NMR (400 MHz, DMSO-d₆) δ 10.75 (s, 1H), 9.01 (s, 1H), 8.60 (d, J=2.5 Hz, 1H), 8.22 (d, J=2.5 Hz, 1H), 8.02 (s, 1H), 7.84 (d, J=4.9 Hz, 1H), 7.38 (d, J=8.8 Hz, 1H), 7.05 (s, 1H), 6.47 (dd, J=10.7, 8.9 Hz, 1H), 6.26 (s, 1H), 5.88 (s, 1H), 4.49 (s, 2H), 4.15 (dd, J=8.8, 5.1 Hz, 1H), 3.98 (s, 2H), 3.77 (s, 3H), 3.37 (dd, J=19.0, 10.9 Hz, 2H), 3.16 (t, J=11.2 Hz, 1H), 3.08-2.99 (m, 1H), 2.87 (dd, J=29.8, 11.8 Hz, 2H), 2.76-2.50 (m, 8H), 2.36-1.92 (m, 3H), 1.64 (dd, J=12.0, 7.9 Hz, 1H), 1.49 (d, J=6.9 Hz, 7H), 0.90 (d, J=6.1 Hz, 3H).

### Example 158. 2-((6-((5-chloro-2-(9-((4-(3-(2,6-dioxopiperidin-3-yl)-1-methyl-1H-indazol-6-yl)piperazin-1-yl)methyl)-3-azaspiro[5.5]undecan-3-yl)pyrimidin-4-yl)amino)-1-isopropyl-2-oxo-1,2-dihydro-1,8-naphthyridin-3-yl)oxy)-N-methylacetamide (Compound 204)

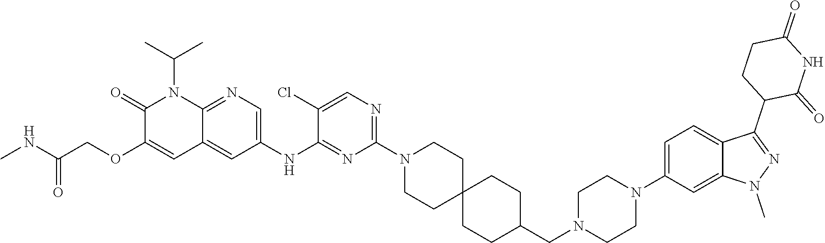 [SMILES: CNC(=O)COc1cc2cc(Nc3nc(N4CCC5(CCC(CN6CCN(c7ccc8c(C9CCC(=O)NC9=O)nn(C)c8c7)CC6)CC5)CC4)ncc3Cl)cnc2n(C(C)C)c1=O]

### Preparation of tert-butyl 9-((4-(3-(2,6-dioxopiperidin-3-yl)-1-methyl-1H-indazol-6-yl)piperazin-1-yl)methyl)-3-azaspiro[5.5]undecane-3-carboxylate

A mixture of tert-butyl 9-formyl-3-azaspiro[5.5]undecane-3-carboxylate (835 mg, 2.97 mmol), 3-(1-methyl-6-(piperazin-1-yl)-1H-indazol-3-yl)piperidine-2,6-dione hydrochloride (720 mg, 1.98 mmol), and potassium acetate (583 mg, 5.94 mmol) was suspended in THF (10.0 mL) and DMF (1.50 mL). The mixture was stirred at room temperature for 20 min. The white mixture was then treated all at once with sodium triacetoxyborohydride (629 mg, 2.97 mmol), and the reaction mixture was stirred at room temperature for 24 h. The reaction mixture was treated with saturated aqueous ammonium chloride solution (~5 mL) and then extracted with ethyl acetate (3×). The combined organics were dried over sodium sulfate, filtered, and concentrated under reduced pressure. The residue was purified by column chromatography (0-100% (3:1 ethyl acetate/EtOH)/heptane) to afford tert-butyl 9-((4-(3-(2,6-dioxopiperidin-3-yl)-1-methyl-1H-indazol-6-yl)piperazin-1-yl)methyl)-3-azaspiro[5.5]undecane-3-carboxylate (819 mg). LC-MS (ESI): m/z=593.2 [M+H]⁺.

### Preparation of 3-(6-(4-((3-azaspiro[5.5]undecan-9-yl)methyl)piperazin-1-yl)-1-methyl-1H-indazol-3-yl)piperidine-2,6-dione bis(trifluoroacetate)

A solution of tert-butyl 9-((4-(3-(2,6-dioxopiperidin-3-yl)-1-methyl-1H-indazol-6-yl)piperazin-1-yl)methyl)-3-azaspiro[5.5]undecane-3-carboxylate (816.4 mg, 1.00 equiv., 1.377 mmol) and DCM (2.00 mL) was treated with a solution of TFA (2.96 g, 2.00 mL, 18.8 equiv., 26.0 mmol) and DCM (2.00 mL). The mixture was stirred at room temperature for 2 h. The reaction mixture was concentrated under reduced pressure to afford 3-(6-(4-((3-azaspiro[5.5]undecan-9-yl)methyl)piperazin-1-yl)-1-methyl-1H-indazol-3-yl)piperidine-2,6-dione bis(trifluoroacetate) (992 mg). LC-MS (ESI): m/z=493.4 [M+H]⁺.

### Preparation of 2-((6-((5-chloro-2-(9-((4-(3-(2,6-dioxopiperidin-3-yl)-1-methyl-1H-indazol-6-yl)piperazin-1-yl)methyl)-3-azaspiro[5.5]undecan-3-yl)pyrimidin-4-yl)amino)-1-isopropyl-2-oxo-1,2-dihydro-1,8-naphthyridin-3-yl)oxy)-N-methylacetamide

A mixture of 2-((6-((2,5-dichloropyrimidin-4-yl)amino)-1-isopropyl-2-oxo-1,2-dihydro-1,8-naphthyridin-3-yl)oxy)-N-methylacetamide (47.8 mg, 109 μmol), 3-(6-(4-((3-azaspiro[5.5]undecan-9-yl)methyl)piperazin-1-yl)-1-methyl-1H-indazol-3-yl)piperidine-2,6-dione bis(trifluoroacetate) (60.0 mg, 83.3 μmol), and CsF (55.0 mg, 362 μmol) in DMSO (1.30 mL) was treated with DIEA (87.0 μL, 500 μmol). The mixture was warmed to 80° C. for 15 h, then cooled to room temperature. The reaction mixture was filtered and purified by prep-HPLC to afford 2-((6-((5-chloro-2-(9-((4-(3-(2,6-dioxopiperidin-3-yl)-1-methyl-1H-indazol-6-yl)piperazin-1-yl)methyl)-3-azaspiro[5.5]undecan-3-yl)pyrimidin-4-yl)amino)-1-isopropyl-2-oxo-1,2-dihydro-1,8-naphthyridin-3-yl)oxy)-N-methylacetamide (17.6 mg). ¹H NMR (400 MHz, DMSO-d₆) § 10.84 (s, 1H), 9.01 (s, 1H), 8.68 (d, J=2.5 Hz, 1H), 8.30 (d, J=2.6 Hz, 1H), 8.06 (s, 1H), 8.00-7.89 (m, 1H), 7.49 (d, J=8.9 Hz, 1H), 7.07 (s, 1H), 6.91 (d, J=8.9 Hz, 1H), 6.83 (s, 1H), 5.96 (s, 1H), 4.58 (s, 2H), 4.25 (dd, J=9.2, 5.2 Hz, 1H), 3.89 (s, 3H), 3.69-3.53 (m, 5H), 3.24-3.11 (m, 5H), 2.68 (d, J=4.7 Hz, 3H), 2.64-2.59 (m, 3H), 2.36-2.24 (m, 2H), 2.23-2.11 (m, 3H), 1.77-1.65 (m, 3H), 1.57 (d, J=6.9 Hz, 6H), 1.50-1.42 (m, 3H), 1.38-1.26 (m, 2H), 1.19-1.01 (m, 5H). LC-MS (ESI): m/z=447.3 [M+2H]²⁺.

### Example 159. 2-((6-((5-chloro-2-(7-((4-(3-(2,6-dioxopiperidin-3-yl)-1-methyl-1H-indazol-6-yl)piperazin-1-yl)methyl)-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide (Compound 205)

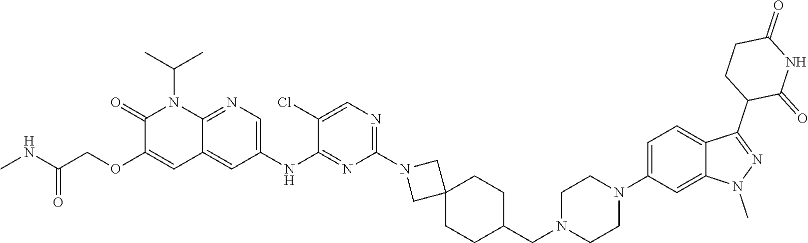 [SMILES: CNC(=O)COc1cc2cc(Nc3nc(N4CC5(CCC(CN6CCN(c7ccc8c(C9CCC(=O)NC9=O)nn(C)c8c7)CC6)CC5)C4)ncc3Cl)cnc2n(C(C)C)c1=O]

### Preparation of tert-butyl 7-((4-(3-(2,6-dioxopiperidin-3-yl)-1-methyl-1H-indazol-6-yl)piperazin-1-yl)methyl)-2-azaspiro[3.5]nonane-2-carboxylate

A mixture of tert-butyl 7-formyl-2-azaspiro[3.5]nonane-2-carboxylate (110 mg, 433 μmol), 3-(1-methyl-6-(piperazin-1-yl)-1H-indazol-3-yl)piperidine-2,6-dione hydrochloride (105 mg, 289 μmol), and potassium acetate (85.0 mg, 866 μmol) suspended in THF (1.50 mL) and DMF (0.225 mL) was stirred at room temperature for 10 min. The white mixture was then treated all at once with sodium triacetoxyborohydride (91.7 mg, 433 μmol), and the reaction mixture was stirred at room temperature for 24 h. The reaction mixture was treated with saturated aqueous ammonium chloride solution (5 mL) and then extracted with ethyl acetate (3×). The combined organics were dried over sodium sulfate, filtered, concentrated under reduced pressure, and purified by column chromatography (0-100% (3:1 ethyl acetate/EtOH)/heptane) to afford tert-butyl 7-((4-(3-(2,6-dioxopiperidin-3-yl)-1-methyl-1H-indazol-6-yl)piperazin-1-yl)methyl)-2-azaspiro[3.5]nonane-2-carboxylate (113.0 mg). LC-MS (ESI): m/z=565.3 [M+H]⁺.

### Preparation of 3-(6-(4-((2-azaspiro[3.5]nonan-7-yl)methyl)piperazin-1-yl)-1-methyl-1H-indazol-3-yl)piperidine-2,6-dione bis(trifluoroacetate)

A solution of tert-butyl 7-((4-(3-(2,6-dioxopiperidin-3-yl)-1-methyl-1H-indazol-6-yl)piperazin-1-yl)methyl)-2-azaspiro[3.5]nonane-2-carboxylate (111.9 mg, 1.00 equiv., 198.1 μmol) and DCM (1.00 mL) was treated with a solution of TFA (3.108 kg, 2100 mL, 1.376e+5 Eq, 27.26 mol) and DCM (1.00 mL). The mixture was stirred at room temperature for 2 h. The volatiles were removed in vacuo to afford 3-(6-(4-((2-azaspiro[3.5]nonan-7-yl)methyl)piperazin-1-yl)-1-methyl-1H-indazol-3-yl)piperidine-2,6-dione bis(trifluoroacetate) (137 mg). LC-MS (ESI): m/z=465.3 [M+H]⁺.

### Preparation of 2-((6-((5-chloro-2-(7-((4-(3-(2,6-dioxopiperidin-3-yl)-1-methyl-1H-indazol-6-yl)piperazin-1-yl)methyl)-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide

A mixture of 2-((6-((2,5-dichloropyrimidin-4-yl)amino)-1-isopropyl-2-oxo-1,2-dihydro-1,8-naphthyridin-3-yl)oxy)-N-methylacetamide (17.4 mg, 39.8 μmol), 3-(6-(4-((2-azaspiro[3.5]nonan-7-yl)methyl)piperazin-1-yl)-1-methyl-1H-indazol-3-yl)piperidine-2,6-dione bis(trifluoroacetate) (20.0 mg, 28.9 μmol), and CsF (30.0 mg, 197 μmol) in DMSO (0.500 mL) was treated with DIEA (30.2 μL, 173 μmol). The mixture was warmed to 80° C., stirred for 15 h, and then cooled to room temperature. The reaction mixture was filtered and purified by prep-HPLC to afford 2-((6-((5-chloro-2-(7-((4-(3-(2,6-dioxopiperidin-3-yl)-1-methyl-1H-indazol-6-yl)piperazin-1-yl)methyl)-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide (12.1 mg). ¹H NMR (400 MHz, DMSO-d₆) § 10.77 (s, 1H), 8.94 (s, 1H), 8.70 (d, J=2.5 Hz, 1H), 8.36 (d, J=2.6 Hz, 1H), 7.98 (s, 1H), 7.94-7.79 (m, 1H), 7.42 (d, J=8.9 Hz, 1H), 7.07 (s, 1H), 6.84 (d, J=9.0 Hz, 1H), 6.76 (s, 1H), 5.89 (s, 1H), 4.51 (s, 2H), 4.18 (dd, J=9.1, 5.1 Hz, 1H), 3.82 (s, 3H), 3.60 (s, 2H), 3.56 (s, 2H), 3.21-3.01 (m, 6H), 2.67-2.59 (m, 4H), 2.57-2.48 (m, 2H), 2.35-2.15 (m, 1H), 2.15-1.99 (m, 4H), 1.88-1.74 (m, 2H), 1.71-1.60 (m, 2H), 1.50 (d, J=6.9 Hz, 6H), 1.48-1.31 (m, 3H), 0.98-0.75 (m, 2H). LC-MS (ESI): m/z=433.3 [M+2H]²⁺.

### Example 160. 2-((6-((5-chloro-2-(9-((4-(3-(2,6-dioxopiperidin-3-yl)-1-methyl-1H-indazol-6-yl)piperidin-1-yl)methyl)-3-azaspiro[5.5]undecan-3-yl)pyrimidin-4-yl)amino)-1-isopropyl-2-oxo-1,2-dihydro-1,8-naphthyridin-3-yl)oxy)-N-methylacetamide (Compound 237)

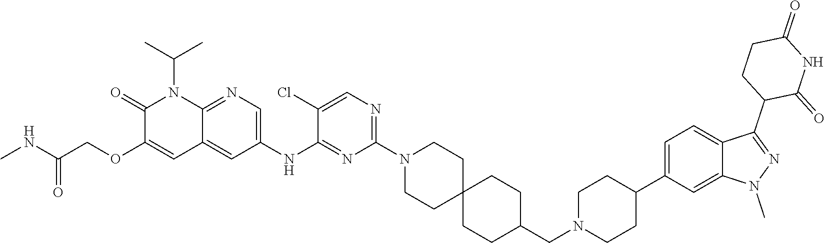 [SMILES: CNC(=O)COc1cc2cc(Nc3nc(N4CCC5(CCC(CN6CCC(c7ccc8c(C9CCC(=O)NC9=O)nn(C)c8c7)CC6)CC5)CC4)ncc3Cl)cnc2n(C(C)C)c1=O]

### Preparation of tert-butyl 9-((4-(3-(2,6-dioxopiperidin-3-yl)-1-methyl-1H-indazol-6-yl)piperidin-1-yl)methyl)-3-azaspiro[5.5]undecane-3-carboxylate

A mixture of tert-butyl 9-formyl-3-azaspiro[5.5]undecane-3-carboxylate (0.927 g, 3.29 mmol),), 3-(1-methyl-6-(piperidin-4-yl)-1H-indazol-3-yl)piperidine-2,6-dione, HCl (1.00 g, 2.76 mmol, prepared using published methods e.g., in International Application Publication No. WO2021255212), and potassium acetate (811 mg, 8.27 mmol) was suspended in THF (10.0 mL) and DMF (1.50 mL). The mixture was stirred at room temperature for 10 min. The white mixture was then treated all at once with sodium triacetoxyborohydride (876 mg, 4.13 mmol), and the reaction mixture was allowed to stir at room temperature for 1 h. The reaction mixture was treated with a saturated aqueous ammonium chloride solution (5 mL), then extracted with ethyl acetate (3×). The combined organic layers were dried over sodium sulfate, filtered, and concentrated under reduced pressure. The residue was purified by column chromatography to afford tert-butyl 9-((4-(3-(2,6-dioxopiperidin-3-yl)-1-methyl-1H-indazol-6-yl)piperidin-1-yl)methyl)-3-azaspiro[5.5]undecane-3-carboxylate (1.245 g). LC-MS (ESI): m/z=592.4 [M+H]⁺.

### Preparation of 3-(6-(1-((3-azaspiro[5.5]undecan-9-yl)methyl)piperidin-4-yl)-1-methyl-1H-indazol-3-yl)piperidine-2,6-dione tris (trifluoroacetate)

A mixture of tert-butyl 9-((4-(3-(2,6-dioxopiperidin-3-yl)-1-methyl-1H-indazol-6-yl)piperidin-1-yl)methyl)-3-azaspiro[5.5]undecane-3-carboxylate (1.244 g, 2.102 mmol) in DCM (2.00 mL) was treated with a solution of TFA (2.00 mL, 26.0 mmol)/DCM (2.00 mL). The mixture was stirred at room temperature for 1 h. The volatiles were removed under reduced pressure to afford 3-(6-(1-((3-azaspiro[5.5]undecan-9-yl)methyl)piperidin-4-yl)-1-methyl-1H-indazol-3-yl)piperidine-2,6-dione tris (trifluoroacetate) (1.75 g), which was used as is without further purification. LC-MS (ESI): m/z=492.4 [M+H]⁺.

### Preparation of 2-((6-((5-chloro-2-(9-((4-(3-(2,6-dioxopiperidin-3-yl)-1-methyl-1H-indazol-6-yl)piperidin-1-yl)methyl)-3-azaspiro[5.5]undecan-3-yl)pyrimidin-4-yl)amino)-1-isopropyl-2-oxo-1,2-dihydro-1,8-naphthyridin-3-yl)oxy)-N-methylacetamide

A mixture of 2-((6-((2,5-dichloropyrimidin-4-yl)amino)-1-isopropyl-2-oxo-1,2-dihydro-1,8-naphthyridin-3-yl)oxy)-N-methylacetamide (55 mg, 0.13 mmol), 3-(6-(1-((3-azaspiro[5.5]undecan-9-yl)methyl)piperidin-4-yl)-1-methyl-1H-indazol-3-yl)piperidine-2,6-dione tris (trifluoroacetate) (97 mg), and CsF (64 mg, 0.42 mmol) in DMSO (2.00 mL) was treated with DIEA (91 μL, 0.52 mmol). The reaction mixture was warmed to 100° C. for 17 h. The reaction mixture was filtered with DMSO and purified by prep-HPLC to afford 2-((6-((5-chloro-2-(9-((4-(3-(2,6-dioxopiperidin-3-yl)-1-methyl-1H-indazol-6-yl)piperidin-1-yl)methyl)-3-azaspiro[5.5]undecan-3-yl)pyrimidin-4-yl)amino)-1-isopropyl-2-oxo-1,2-dihydro-1,8-naphthyridin-3-yl)oxy)-N-methylacetamide (54.3 mg) as a white solid. ¹H NMR (400 MHz, DMSO-d₆) δ 10.87 (s, 1H), 9.02 (s, 1H), 8.68 (d, J=2.4 Hz, 1H), 8.31 (s, 1H), 8.07 (s, 1H), 7.95 (s, 1H), 7.42 (s, 1H), 7.11-6.98 (m, 2H), 6.01-5.88 (m, 1H), 4.58 (s, 2H), 4.39-4.24 (m, 1H), 3.97 (s, 3H), 3.72-3.51 (m, 6H), 3.30-3.21 (m, 4H), 3.15-2.90 (m, 4H), 2.76-2.57 (m, 5H), 2.42-2.24 (m, 3H), 2.21-2.12 (m, 2H), 2.09-1.89 (m, 2H), 1.86-1.65 (m, 3H), 1.57 (d, J=6.9 Hz, 6H), 1.52-1.43 (m, 1H), 1.36-1.27 (m, 2H), 1.26-1.20 (m, 2H), 1.18-1.02 (m, 2H). LC-MS (ESI): m/z=446.8 [M+2H]²⁺

### Example 161. 2-((6-((5-chloro-2-(9-((4-(3-(2,6-dioxopiperidin-3-yl)-7-fluoro-1-methyl-1H-indazol-6-yl)piperidin-1-yl)methyl)-3-azaspiro[5.5]undecan-3-yl)pyrimidin-4-yl)amino)-1-isopropyl-2-oxo-1,2-dihydro-1,8-naphthyridin-3-yl)oxy)-N-methylacetamide (Compound 240)

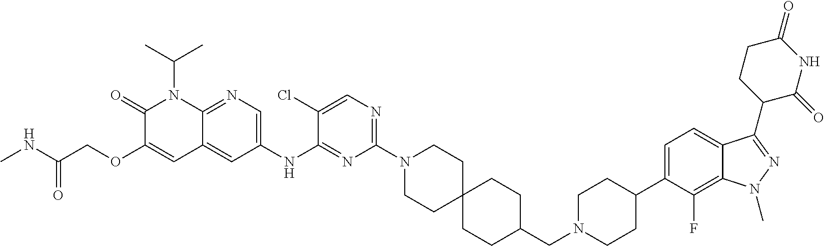 [SMILES: CNC(=O)COc1cc2cc(Nc3nc(N4CCC5(CCC(CN6CCC(c7ccc8c(C9CCC(=O)NC9=O)nn(C)c8c7F)CC6)CC5)CC4)ncc3Cl)cnc2n(C(C)C)c1=O]

### Preparation of tert-butyl 4-(3-(2,6-bis(benzyloxy)pyridin-3-yl)-7-fluoro-1-methyl-1H-indazol-6-yl)-3,6-dihydropyridine-1 (2H)-carboxylate

To a solution of 3-(2,6-bis(benzyloxy)pyridin-3-yl)-6-bromo-7-fluoro-1-methyl-1H-indazole (1.0 g, 1.93 mmol) in 1,4-dioxane:water (20 mL, 9:1) were added tert-butyl 4-(4,4,5,5-tetramethyl-1,3,2-dioxaborolan-2-yl)-3,6-dihydropyridine-1 (2H)-carboxylate (0.71 g, 2.32 mmol) and potassium phosphate (1.6 g, 7.72 mmol). The reaction mixture was purged with argon for 15 min. Then, PdCl₂(dppf) (0.14 g, 0.19 mmol) was added, and the reaction mixture was stirred at 80° C. for 4 h. After completion, the reaction mixture was filtered through a celite pad. The filtrate was diluted with water (100 mL) and extracted with ethyl acetate (2×100 mL). The organic layer was dried over anhydrous Na₂SO₄, filtered, and concentrated under reduced pressure. The crude was purified by silica gel (100-200) flash column chromatography (0-35% ethyl acetate in petroleum ether) to afford tert-butyl 4-(3-(2,6-bis(benzyloxy)pyridin-3-yl)-7-fluoro-1-methyl-1H-indazol-6-yl)-3,6-dihydropyridine-1 (2H)-carboxylate (0.80 g, 60%) as a light green semi solid.

LCMS (ESI): m/z=621.58 (M+H)+

### Preparation of tert-butyl 4-(3-(2,6-dioxopiperidin-3-yl)-7-fluoro-1-methyl-1H-indazol-6-yl)piperidine-1-carboxylate

To a mixture of tert-butyl 4-(3-(2,6-bis(benzyloxy)pyridin-3-yl)-7-fluoro-1-methyl-1H-indazol-6-yl)-3,6-dihydropyridine-1 (2H)-carboxylate (0.80 g, 0.17 mmol) in THF:MeOH (1:1; 32 mL) was added 10% Pd/C (0.8 g; w/w). The reaction mixture was kept in a parr shaker under H₂ atmosphere (60 psi) for 6 h. After completion, the reaction mixture was filtered through a celite pad., and the filtrate was concentrated under reduced pressure to afford tert-butyl 4-(3-(2,6-dioxopiperidin-3-yl)-7-fluoro-1-methyl-1H-indazol-6-yl)piperidine-1-carboxylate (0.53 g, 62%) as an off-white solid.

LCMS (ESI): m/z=445.43 (M+H)⁺

### Preparation of 3-(7-fluoro-1-methyl-6-(piperidin-4-yl)-1H-indazol-3-yl)piperidine-2,6-dione

To a stirred solution of tert-butyl 4-(3-(2,6-dioxopiperidin-3-yl)-7-fluoro-1-methyl-1H-indazol-6-yl)piperidine-1-carboxylate (0.53 g, 5.16 mmol) in DCM (5.3 mL) at 0° C. was added TFA (5.3 mL). The reaction mixture was stirred at room temperature for 2 h. After completion, the reaction mixture was concentrated under reduced pressure. The crude was triturated with diethyl ether and dried under vacuum to afford 3-(7-fluoro-1-methyl-6-(piperidin-4-yl)-1H-indazol-3-yl)piperidine-2,6-dione (0.49 g, 92%) as an off-white solid LCMS (ESI): m/z=345.38 (M+H)+

### Preparation of tert-butyl 9-((4-(3-(2,6-dioxopiperidin-3-yl)-7-fluoro-1-methyl-1H-indazol-6-yl)piperidin-1-yl)methyl)-3-azaspiro[5.5]undecane-3-carboxylate

A mixture of tert-butyl 9-formyl-3-azaspiro[5.5]undecane-3-carboxylate (257 mg, 914 μmol), 3-(7-fluoro-1-methyl-6-(piperidin-4-yl)-1H-indazol-3-yl)piperidine-2,6-dione, HCl (290 mg, 761 μmol), and potassium acetate (224 mg, 2.28 mmol) was suspended in THF (5.00 mL) and DMF (0.750 mL). The mixture was stirred at room temperature for 15 min. The white mixture was then treated all at once with sodium triacetoxyborohydride (242 mg, 1.14 mmol), and the reaction mixture was allowed to stir at room temperature for 1 h. The reaction mixture was treated with saturated aqueous ammonium chloride solution (5 mL) and then extracted with DCM (3×). The combined organics were dried over sodium sulfate, filtered, and concentrated under reduced pressure. The residue was purified by column chromatography to afford tert-butyl 9-((4-(3-(2,6-dioxopiperidin-3-yl)-7-fluoro-1-methyl-1H-indazol-6-yl)piperidin-1-yl)methyl)-3-azaspiro[5.5]undecane-3-carboxylate (378 mg). LC-MS (ESI): m/z=610.4 [M+H]⁺.

### Preparation of 3-(6-(1-((3-azaspiro[5.5]undecan-9-yl)methyl)piperidin-4-yl)-7-fluoro-1-methyl-1H-indazol-3-yl)piperidine-2,6-dione dihydrochloride

A solution of tert-butyl 9-((4-(3-(2,6-dioxopiperidin-3-yl)-7-fluoro-1-methyl-1H-indazol-6-yl)piperidin-1-yl)methyl)-3-azaspiro[5.5]undecane-3-carboxylate (377 mg, 618 μmol) in dioxane (2.00 mL) was treated with HCl (4.0 M in dioxane) (4.00 mL, 16.0 mmol). The mixture was stirred at room temperature for 3 h. The volatiles were removed under reduced pressure to afford 3-(6-(1-((3-azaspiro[5.5]undecan-9-yl)methyl)piperidin-4-yl)-7-fluoro-1-methyl-1H-indazol-3-yl)piperidine-2,6-dione dihydrochloride (381 mg), which was used as is without further purification. LC-MS (ESI): m/z=510.3 [M+H]⁺.

### Preparation of 2-((6-((5-chloro-2-(9-((4-(3-(2,6-dioxopiperidin-3-yl)-7-fluoro-1-methyl-1H-indazol-6-yl)piperidin-1-yl)methyl)-3-azaspiro[5.5]undecan-3-yl)pyrimidin-4-yl)amino)-1-isopropyl-2-oxo-1,2-dihydro-1,8-naphthyridin-3-yl)oxy)-N-methylacetamide

A mixture of 2-((6-((2,5-dichloropyrimidin-4-yl)amino)-1-isopropyl-2-oxo-1,2-dihydro-1,8-naphthyridin-3-yl)oxy)-N-methylacetamide (176 mg, 402 μmol), 3-(6-(1-((3-azaspiro[5.5]undecan-9-yl)methyl)piperidin-4-yl)-7-fluoro-1-methyl-1H-indazol-3-yl)piperidine-2,6-dione dihydrochloride (180 mg, 309 μmol), and CsF (188 mg, 1.24 mmol) in DMSO (2.00 mL) was treated with DIEA (269 μL, 1.54 mmol). The reaction mixture was warmed to 80° C. for 24 h and then cooled to room temperature. The reaction mixture was filtered with DMSO and purified by prep-HPLC to afford 2-((6-((5-chloro-2-(9-((4-(3-(2,6-dioxopiperidin-3-yl)-7-fluoro-1-methyl-1H-indazol-6-yl)piperidin-1-yl)methyl)-3-azaspiro[5.5]undecan-3-yl)pyrimidin-4-yl)amino)-1-isopropyl-2-oxo-1,2-dihydro-1,8-naphthyridin-3-yl)oxy)-N-methylacetamide (67.6 mg) as an off-white solid. ¹H NMR (400 MHz, DMSO-d₆) δ 10.91 (s, 1H), 9.02 (s, 1H), 8.69 (d, J=2.4 Hz, 1H), 8.31 (d, J=2.5 Hz, 1H), 8.07 (s, 1H), 8.00-7.90 (m, 1H), 7.46 (d, J=8.4 Hz, 1H), 7.12-7.00 (m, 2H), 6.04-5.85 (m, 1H), 4.58 (s, 2H), 4.36 (dd, J=10.3, 5.0 Hz, 1H), 4.11 (s, 3H), 3.72-3.51 (m, 4H), 3.32-3.25 (m, 3H), 3.03-2.84 (m, 3H), 2.72-2.62 (m, 5H), 2.38-2.29 (m, 2H), 2.24-2.11 (m, 3H), 2.09-1.93 (m, 1H), 1.86-1.74 (m, 1H), 1.74-1.65 (m, 3H), 1.57 (d, J=6.9 Hz, 7H), 1.50-1.43 (m, 2H), 1.36-1.29 (m, 2H), 1.27-1.20 (m, 2H), 1.17-1.00 (m, 3H). LC-MS (ESI): m/z=455.9 [M+2H]²⁺

### Example 162. 2-[[6-[[5-chloro-2-[(6R,7R)-7-[3-(2,6-dioxo-3-piperidyl)-1-methyl-indazol-6-yl]oxy-6-methyl-2-azaspiro[3.5]nonan-2-yl]pyrimidin-4-yl]amino]-1-isopropyl-2-oxo-1,8-naphthyridin-3-yl]oxy]-N-methyl-acetamide [rel-2-((6-((5-chloro-2-((6R,7R)-7-((3-(2,6-dioxopiperidin-3-yl)-1-methyl-1H-indazol-6-yl)oxy)-6-methyl-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide] (Compound 185d)

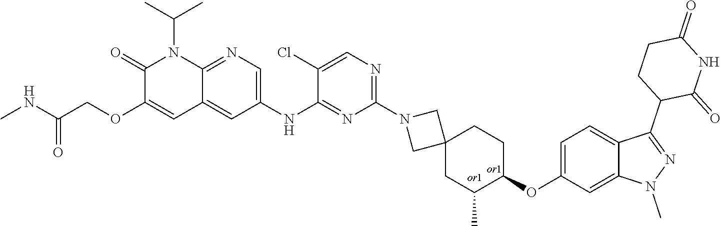 [SMILES: CNC(=O)COc1cc2cc(Nc3nc(N4CC5(CC[C@@H](Oc6ccc7c(C8CCC(=O)NC8=O)nn(C)c7c6)[C@H](C)C5)C4)ncc3Cl)cnc2n(C(C)C)c1=O]

### Preparation of 2-[[6-[[5-chloro-2-[(6R,7R)-7-[3-(2,6-dioxo-3-piperidyl)-1-methyl-indazol-6-yl]oxy-6-methyl-2-azaspiro[3.5]nonan-2-yl]pyrimidin-4-yl]amino]-1-isopropyl-2-oxo-1,8-naphthyridin-3-yl]oxy]-N-methyl-acetamide [rel-2-((6-((5-chloro-2-((6R,7R)-7-((3-(2,6-dioxopiperidin-3-yl)-1-methyl-1H-indazol-6-yl)oxy)-6-methyl-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide]

To a solution of 2-[6-[[(2,5-dichloropyrimidin-4-yl)amino]-1-isopropyl-2-oxo-1,8-naphthyridin-3-yl]oxy]-N-methyl-acetamide (100 mg, 228.69 μmol, 1 equiv) in DMSO (1 mL) was added TEA (92.56 mg, 914.75 μmol, 127.32 μL, 4 equiv) and 3-[1-methyl-6-[[(6R,7R)-6-methyl-2-azaspiro[3.5]nonan-7-yl]oxy] indazol-3-yl]piperidine-2,6-dione (136.01 mg, 343.03 μmol, 1.5 equiv). The mixture was stirred at 100° C. for 1 h. The reaction mixture was filtered to give a residue. The residue was purified by prep-HPLC to afford 2-[[6-[[5-chloro-2-[(6R,7R)-7-[3-(2,6-dioxo-3-piperidyl)-1-methyl-indazol-6-yl]oxy-6-methyl-2-azaspiro[3.5]nonan-2-yl]pyrimidin-4-yl]amino]-1-isopropyl-2-oxo-1,8-naphthyridin-3-yl]oxy]-N-methyl-acetamide [rel-2-((6-((5-chloro-2-((6R,7R)-7-((3-(2,6-dioxopiperidin-3-yl)-1-methyl-1H-indazol-6-yl)oxy)-6-methyl-2-azaspiro[3.5]nonan-2-yl)pyrimidin-4-yl)amino)-1-isopropyl-2-oxo-1,2-dihydro-1,8-naphthyridin-3-yl)oxy)-N-methylacetamide] (1.64 mg, 2.06 μmol) as a yellow solid.

LC-MS (ESI): m/z=797.4 [M+H]⁺.

¹H NMR (400 MHz, DMSO-d₆) δ=10.89-10.83 (m, 1H), 9.03 (s, 1H), 8.78 (s, 1H), 8.41 (s, 1H), 8.07 (s, 1H), 7.96 (d, J=3.2 Hz, 1H), 7.55 (d, J=8.4 Hz, 1H), 7.18 (s, 1H), 7.09 (s, 1H), 6.74 (d, J=10.0 Hz, 1H), 6.04-5.92 (m, 1H), 4.59 (s, 2H), 4.34-4.24 (m, 1H), 4.09-4.01 (m, 1H), 3.93 (s, 3H), 3.81-3.76 (m, 1H), 3.69 (s, 2H), 2.70-2.69 (m, 4H), 2.18-2.10 (m, 2H), 2.02-1.95 (m, 2H), 1.77-1.67 (m, 2H), 1.58 (d, J=7.2 Hz, 6H), 1.49-1.38 (m, 2H), 1.28-1.22 (m, 3H), 1.15 (s, 1H), 0.99 (s, 2H).

### Example 163: 2-((6-((5-chloro-2-((3S,4S)-4-((4-(3-(2,6-dioxopiperidin-3-yl)-1-methyl-1H-indazol-7-yl)piperazin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide [rel-2-((6-((5-chloro-2-((3S,4S)-4-((4-(3-(2,6-dioxopiperidin-3-yl)-1-methyl-1H-indazol-7-yl)piperazin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide] (Compound 238a)

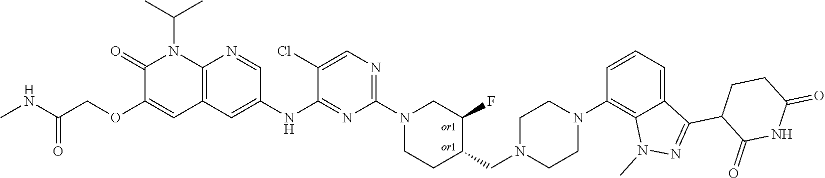 [SMILES: CNC(=O)COc1cc2cc(Nc3nc(N4CC[C@@H](CN5CCN(c6cccc7c(C8CCC(=O)NC8=O)nn(C)c67)CC5)[C@H](F)C4)ncc3Cl)cnc2n(C(C)C)c1=O]

### Preparation of trans-tert-butyl 4-(4-(3-(2-(benzyloxy)-6-hydroxypyridin-3-yl)-1-methyl-1H-indazol-7-yl)piperazine-1-carbonyl)-3-fluoropiperidine-1-carboxylate

To a stirred solution of 6-(benzyloxy)-5-(1-methyl-7-(piperazin-1-yl)-1H-indazol-3-yl)pyridin-2-ol (1.60 g, 3.85 mmol) and trans-1-(tert-butoxycarbonyl)-3-fluoropiperidine-4-carboxylic acid (0.95 g, 3.85 mmol) in THF (32.0 mL) was added DIPEA (2.0 mL, 11.55 mmol). The reaction mixture was stirred for 10 minutes. To this mixture was added propylphosphonic anhydride (6.12 g, 19.25 mmol) at 0° C. The mixture was stirred at room temperature for 3 h. The reaction mixture was quenched with cold water (40 mL) and extracted with ethyl acetate (3×60 mL). The combined organic layer was dried over anhydrous Na₂SO₄, filtered, and concentrated under reduced pressure to obtain a crude product which was purified by flash chromatography (50-100% ethyl acetate in petroleum ether) to afford trans-tert-butyl 4-(4-(3-(2-(benzyloxy)-6-hydroxypyridin-3-yl)-1-methyl-1H-indazol-7-yl)piperazine-1-carbonyl)-3-fluoropiperidine-1-carboxylate (1.30 g) as a pale-yellow solid.

LC-MS (ESI): m/z=645.35 [M+H]⁺

### Preparation of trans-tert-butyl-4-((4-(3-(2-(benzyloxy)-6-hydroxypyridin-3-yl)-1-methyl-1H-indazol-7-yl)piperazin-1-yl)methyl)-3-fluoropiperidine-1-carboxylate

To a stirred solution of trans-tert-butyl 4-(4-(3-(2-(benzyloxy)-6-hydroxypyridin-3-yl)-1-methyl-1H-indazol-7-yl)piperazine-1-carbonyl)-3-fluoropiperidine-1-carboxylate (1.30 g, 2.01 mmol) in THF (26.0 mL) was added BH₃.DMS (neat) (6.5 mL) at 0° C. The mixture was stirred at room temperature for 16 h. The reaction mixture was quenched with MeOH (30 mL) and concentrated and co-distilled with methanol (30 mL×2) to obtain crude (as a Borane DMS complex). To this residue was added MeOH (150 mL) and refluxed at 80° C. for 6 h. The mixture was cooled to room temperature, concentrated, and dried to afford trans-tert-butyl-4-((4-(3-(2-(benzyloxy)-6-hydroxypyridin-3-yl)-1-methyl-1H-indazol-7-yl)piperazin-1-yl)methyl)-3-fluoropiperidine-1-carboxylate (1.20 g) as an off white solid.

LC-MS (ESI): m/z=631.67 [M+H]⁺

### Preparation of tert-butyl (3S,4S)-4-((4-(3-(2-(benzyloxy)-6-hydroxypyridin-3-yl)-1-methyl-1H-indazol-7-yl)piperazin-1-yl)methyl)-3-fluoropiperidine-1-carboxylate and tert-butyl (3R,4R)-4-((4-(3-(2-(benzyloxy)-6-hydroxypyridin-3-yl)-1-methyl-1H-indazol-7-yl)piperazin-1-yl)methyl)-3-fluoropiperidine-1-carboxylate

Stereoisomers of trans-tert-butyl-4-((4-(3-(2-(benzyloxy)-6-hydroxypyridin-3-yl)-1-methyl-1H-indazol-7-yl)piperazin-1-yl)methyl)-3-fluoropiperidine-1-carboxylate (1.20 g) were separated by chiral SFC purification to afford tert-butyl (3S,4S)-4-((4-(3-(2-(benzyloxy)-6-hydroxypyridin-3-yl)-1-methyl-1H-indazol-7-yl)piperazin-1-yl)methyl)-3-fluoropiperidine-1-carboxylate as an off white solid (0.60 g) and tert-butyl (3R,4R)-4-((4-(3-(2-(benzyloxy)-6-hydroxypyridin-3-yl)-1-methyl-1H-indazol-7-yl)piperazin-1-yl)methyl)-3-fluoropiperidine-1-carboxylate as an off white solid (0.50 g).

### Preparation of tert-butyl (3S,4S)-4-((4-(3-(2,6-dioxopiperidin-3-yl)-1-methyl-1H-indazol-7-yl)piperazin-1-yl)methyl)-3-fluoropiperidine-1-carboxylate

To a stirred solution of tert-butyl (3S,4S)-4-((4-(3-(2-(benzyloxy)-6-hydroxypyridin-3-yl)-1-methyl-1H-indazol-7-yl)piperazin-1-yl)methyl)-3-fluoropiperidine-1-carboxylate (0.60 g, 0.95 mmol.) in THF (60.0 mL) was added 20% palladium hydroxide (0.72 g, 120% w/w). The mixture was stirred under H₂ (80 psi) pressure for 16 h at room temperature. The reaction mixture was diluted with THF (80 mL), filtered through a celite bed, and washed with THF:DCM (1:1, 200 mL). The filtrate was concentrated and dried to obtain crude product which was triturated with diethyl ether, and dried to afford tert-butyl (3S,4S)-4-((4-(3-(2,6-dioxopiperidin-3-yl)-1-methyl-1H-indazol-7-yl)piperazin-1-yl)methyl)-3-fluoropiperidine-1-carboxylate (0.40 g) as a light brown semi-solid.

LC-MS (ESI): m/z=543.57 [M+H]⁺

### Preparation of 3-(7-(4-(((3S,4S)-3-fluoropiperidin-4-yl)methyl)piperazin-1-yl)-1-methyl-1H-indazol-3-yl)piperidine-2,6-dione

To a stirred solution of tert-butyl (3S,4S)-4-((4-(3-(2,6-dioxopiperidin-3-yl)-1-methyl-1H-indazol-7-yl)piperazin-1-yl)methyl)-3-fluoropiperidine-1-carboxylate (0.40 g, 0.74 mmol) in DCM (12.0 mL) was added TFA (2.0 mL) at 0° C. The mixture was stirred at room temperature for 2 h. The reaction mixture was concentrated under reduced pressure and co-distilled with DCM (2×20 mL) to obtain crude product which was triturated with diethyl ether (2×10 mL) and dried to afford 3-(7-(4-(((3S,4S)-3-fluoropiperidin-4-yl)methyl)piperazin-1-yl)-1-methyl-1H-indazol-3-yl)piperidine-2,6-dione (0.41 g) as a pale brown solid.

LC-MS (ESI): m/z=443.54 [M+H]⁺

### Preparation of 2-((6-((5-chloro-2-((3S,4S)-4-((4-(3-(2,6-dioxopiperidin-3-yl)-1-methyl-1H-indazol-7-yl)piperazin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide [rel-2-((6-((5-chloro-2-((3S,4S)-4-((4-(3-(2,6-dioxopiperidin-3-yl)-1-methyl-1H-indazol-7-yl)piperazin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide]

To a stirred solution of 3-(7-(4-(((3S,4S)-3-fluoropiperidin-4-yl)methyl)piperazin-1-yl)-1-methyl-1H-indazol-3-yl)piperidine-2,6-dione (0.20 g, 0.45 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.08 g, 0.18 mmol) in DMSO (2.0 mL) was added DIPEA (2.0 mL). The mixture was stirred at 100° C. for 6 h. The reaction mixture was cooled to room temperature and treated with ice water. The precipitated solid was filtered and dried to obtain a crude product which was purified by prep HPLC to afford 2-((6-((5-chloro-2-((3S,4S)-4-((4-(3-(2,6-dioxopiperidin-3-yl)-1-methyl-1H-indazol-7-yl)piperazin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide [rel-2-((6-((5-chloro-2-((3S,4S)-4-((4-(3-(2,6-dioxopiperidin-3-yl)-1-methyl-1H-indazol-7-yl)piperazin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide](0.049 g) as an off white solid.

LC-MS (ESI): m/z=843.50 [M+H]⁺

¹H NMR (400 MHz, DMSO-d₆): δ 10.88 (s, 1H), 9.13 (s, 1H), 8.68 (d, J=2.40 Hz, 1H), 8.28 (d, J=2.40 Hz, 1H), 8.11 (s, 1H), 7.94 (d, J=4.80 Hz, 1H), 7.39 (dd, J=1.60, 7.00 Hz, 1H), 7.11 (s, 1H), 7.05-7.00 (m, 2H), 5.95 (brs, 1H), 4.56 (s, 2H), 4.46-4.31 (m, 3H), 4.24 (s, 3H), 4.14-4.06 (m, 1H), 3.21-2.75 (m, 8H), 2.68-2.58 (m, 7H), 2.39-2.27 (m, 2H), 2.19-1.99 (m, 4H), 1.57 (d, J=7.20 Hz, 6H), 1.25-1.23 (m, 1H).

### Example 164: 2-((6-((5-chloro-2-((3R,4R)-4-((4-(3-(2,6-dioxopiperidin-3-yl)-1-methyl-1H-indazol-7-yl)piperazin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide [rel-2-((6-((5-chloro-2-((3R,4R)-4-((4-(3-(2,6-dioxopiperidin-3-yl)-1-methyl-1H-indazol-7-yl)piperazin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide] (Compound 238b)

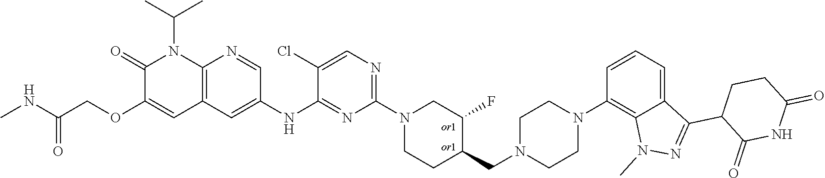 [SMILES: CNC(=O)COc1cc2cc(Nc3nc(N4CC[C@H](CN5CCN(c6cccc7c(C8CCC(=O)NC8=O)nn(C)c67)CC5)[C@@H](F)C4)ncc3Cl)cnc2n(C(C)C)c1=O]

### Preparation of tert-butyl (3R,4R)-4-((4-(3-(2,6-dioxopiperidin-3-yl)-1-methyl-1H-indazol-7-yl)piperazin-1-yl)methyl)-3-fluoropiperidine-1-carboxylate

To a stirred solution of tert-butyl (3R,4R)-4-((4-(3-(2-(benzyloxy)-6-hydroxypyridin-3-yl)-1-methyl-1H-indazol-7-yl)piperazin-1-yl)methyl)-3-fluoropiperidine-1-carboxylate (0.50 g, 0.79 mmol) in THF (50.0 mL) was added 20% palladium hydroxide (0.50 g, 100% w/w). The mixture was stirred under H₂ (80 psi) pressure for 16 h at room temperature. The reaction mixture was diluted with THF (50 mL), filtered through a celite bed, and washed with THF:DCM (1:1, 200 mL). The filtrate was concentrated and dried to obtain a crude product which was triturated with diethyl ether, and dried to afford tert-butyl (3R,4R)-4-((4-(3-(2,6-dioxopiperidin-3-yl)-1-methyl-1H-indazol-7-yl)piperazin-1-yl)methyl)-3-fluoropiperidine-1-carboxylate (0.44 g) as a brown semisolid.

LC-MS (ESI): m/z=543.57 [M+H]⁺

### Preparation of 3-(7-(4-(((3R,4R)-3-fluoropiperidin-4-yl)methyl)piperazin-1-yl)-1-methyl-1H-indazol-3-yl)piperidine-2,6-dione

To a stirred solution of tert-butyl (3R,4R)-4-((4-(3-(2,6-dioxopiperidin-3-yl)-1-methyl-1H-indazol-7-yl)piperazin-1-yl)methyl)-3-fluoropiperidine-1-carboxylate (0.44 g, 0.81 mmol) in DCM (13.2 mL) was added TFA (2.20 mL) at 0° C. The mixture was stirred at room temperature for 2 h. The reaction mixture was concentrated under reduced pressure and co-distilled with DCM (3×30 mL) to obtain crude product which was triturated with diethyl ether (3×10 mL), and dried to afford 3-(7-(4-(((3R,4R)-3-fluoropiperidin-4-yl)methyl)piperazin-1-yl)-1-methyl-1H-indazol-3-yl)piperidine-2,6-dione (0.40 g) as an off white solid.

LC-MS (ESI): m/z=443.24 [M+H]⁺

### Preparation of 2-((6-((5-chloro-2-((3R,4R)-4-((4-(3-(2,6-dioxopiperidin-3-yl)-1-methyl-1H-indazol-7-yl)piperazin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide

To a stirred solution of 3-(7-(4-(((3R,4R)-3-fluoropiperidin-4-yl)methyl)piperazin-1-yl)-1-methyl-1H-indazol-3-yl)piperidine-2,6-dione (0.20 g, 0.45 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.08 g, 0.18 mmol) in DMSO (2.0 mL) was added DIPEA (2.0 mL). The mixture was stirred at 100° C. for 6 h. The reaction mixture was cooled to room temperature and treated with ice water. The precipitated solid was filtered and dried to obtain a crude product which was purified by prep HPLC to afford 2-((6-((5-chloro-2-((3R,4R)-4-((4-(3-(2,6-dioxopiperidin-3-yl)-1-methyl-1H-indazol-7-yl)piperazin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide (0.051 g) as an off white solid.

LC-MS (ESI): m/z=843.55 [M+H]⁺

¹H NMR (400 MHz, DMSO-d₆): δ 10.89 (s, 1H), 9.21 (s, 2H), 8.71 (d, J=2.40 Hz, 1H), 8.25 (d, J=2.00 Hz, 1H), 8.15 (s, 1H), 7.96 (d, J=4.80 Hz, 1H), 7.49 (d, J=8.00 Hz, 1H), 7.21-7.05 (m, 3H), 5.99 (br s, 1H), 4.57 (s, 4H), 4.38-4.26 (m, 5H), 3.71-3.68 (m, 2H), 3.36-3.14 (m, 9H), 2.68-2.62 (m, 5H), 2.50-2.31 (m, 3H), 2.17 (q, J=5.60 Hz, 1H), 2.05-1.96 (m, 1H), 1.58 (d, J=6.80 Hz, 6H), 1.36-1.34 (m, 1H).

### Example 165. 2-((6-((5-chloro-2-(9-((1-(3-(2,6-dioxopiperidin-3-yl)-5-fluoro-1-methyl-1H-indazol-6-yl)piperidin-4-yl)methyl)-3,9-diazaspiro[5.5]undecan-3-yl)pyrimidin-4-yl)amino)-1-isopropyl-2-oxo-1,2-dihydro-1,8-naphthyridin-3-yl)oxy)-N-methylacetamide (Compound 239)

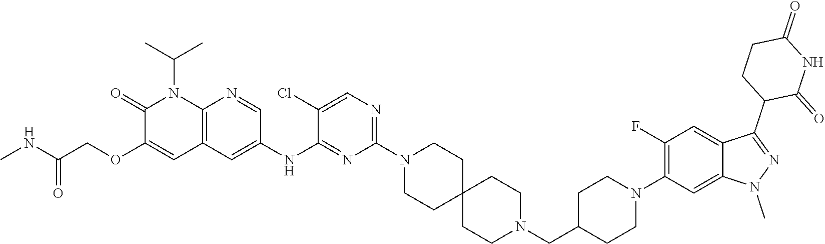 [SMILES: CNC(=O)COc1cc2cc(Nc3nc(N4CCC5(CCN(CC6CCN(c7cc8c(cc7F)c(C7CCC(=O)NC7=O)nn8C)CC6)CC5)CC4)ncc3Cl)cnc2n(C(C)C)c1=O]

### Preparation of 6-bromo-5-fluoro-1H-indazole

To a stirred solution of 4-bromo-2,5-difluorobenzaldehyde (5.00 g, 22.62 mmol) in DME (50 mL), methoxyamine hydrochloride (2.00 g, 23.98 mmol) and potassium carbonate (3.00 g, 21.72 mmol) were added. The mixture was heated at 40° C. for 3 h. The mixture was diluted with DME (100 mL) and filtered through a pad of celite and washed with excess of ethyl acetate (100 mL). The collected filtrate was concentrated under vacuum to obtain a crude product. Hydrazine monohydrate, 98+% (12.5 mL) in DME (50 mL) was added to the above crude product and heated to 90° C. for 18 h. The reaction mixture was concentrated under reduced pressure to afford crude product which was purified by flash chromatography (10-15% of ethyl acetate in petroleum ether) to obtain 6-bromo-5-fluoro-1H-indazole (1.30 g) as a pale-yellow solid.

LC-MS (ESI): m/z=214.85 [M+H]⁺

### Preparation of 6-bromo-5-fluoro-3-iodo-1H-indazole

To stirred solution of 6-bromo-5-fluoro-1H-indazole (1.30 g, 6.05 mmol) in THF (25 mL), were added iodine (3.07 g, 12.09 mmol) and potassium hydroxide (1.70 g, 30.23 mmol) at room temperature. The mixture was stirred at room temperature for 2 h. The reaction mixture was concentrated under reduced pressure to afford a crude product which was triturated with n-pentane to afford 6-bromo-5-fluoro-3-iodo-1H-indazole (0.90 g) as a pale-brown solid.

LC-MS (ESI): m/z=340.84 [M+H]⁺

### Preparation of 6-bromo-5-fluoro-3-iodo-1-methyl-1H-indazole

To a stirred solution of 6-bromo-5-fluoro-3-iodo-1H-indazole (0.90 g, 2.64 mmol) in THF (10 mL) were added potassium tert-butoxide (0.89 g, 7.92 mmol) and iodomethane (1 mL) at 0° C. The mixture was stirred at room temperature for 4 h. The reaction mixture was quenched with water (50 mL) and extracted with ethyl acetate (100 mL). The combined organic layers were washed with brine, dried over anhydrous Na₂SO₄, filtered, and concentrated under vacuum to obtain a crude product which was purified by flash chromatography (10-15% of ethyl acetate in petroleum ether) to obtain 6-bromo-5-fluoro-3-iodo-1-methyl-1H-indazole as an off-white solid (0.45 g).

LC-MS (ESI): m/z=354.84 [M+H]⁺

### Preparation of 3-(2,6-bis(benzyloxy)pyridin-3-yl)-6-bromo-5-fluoro-1-methyl-1H-indazole

To a stirred solution of 6-bromo-5-fluoro-3-iodo-1-methyl-1H-indazole (0.45 g, 1.27 mmol) and (2,6-bis(benzyloxy)pyridin-3-yl) boronic acid (0.51 g, 1.52 mmol) in dioxane (9 mL) and water (0.90 mL) was added K₃PO₄ (0.81 g, 3.80 mmol). The mixture was purged with N₂ for 15 min followed by the addition of Pd(PPh₃)₄ (0.15 g, 0.13 mmol). The mixture was heated at 100° C. and stirred for 6 h. The reaction mixture was cooled to room temperature, diluted with ethyl acetate (100 mL), filtered through a celite bed, and washed with DCM (100 mL). The filtrate was concentrated to get a crude product which was purified by flash chromatography (5-10% of ethyl acetate in petroleum ether) to obtain 3-(2,6-bis(benzyloxy)pyridin-3-yl)-6-bromo-5-fluoro-1-methyl-1H-indazole as a pale-yellow solid (0.45 g).

LC-MS (ESI): m/z=520.35 [M+H]⁺

### Preparation of tert-butyl 9-((1-(3-(2,6-bis(benzyloxy)pyridin-3-yl)-5-fluoro-1-methyl-1H-indazol-6-yl)piperidin-4-yl)methyl)-3,9-diazaspiro[5.5]undecane-3-carboxylate

To a solution of 3-(2,6-bis(benzyloxy)pyridin-3-yl)-6-bromo-5-fluoro-1-methyl-1H-indazole (1.00 g, 1 equiv, 1.93 mmol) in 1,4-dioxane (8 mL) was added tert-butyl 9-(piperidin-4-ylmethyl)-3,9-diazaspiro[5.5]undecane-3-carboxylate (746 mg, 1.1 equiv, 2.12 mmol), sodium tert-butoxide (556 mg, 2.89 mL, 2.0 molar, 3.0 equiv, 5.79 mmol), and (2-dicyclohexylphosphino-2′,6′-diisopropoxy-1,l′-biphenyl) [2-(2′-amino-1,1′-biphenyl)]palladium (II) methanesulfonate (121 mg, 0.075 equiv, 145 μmol). The reaction vial was sealed and heated to 100° C. for 1 h. The reaction mixture was partitioned between ethyl acetate and saturated ammonium chloride (aq). The organic layer was removed, and the aqueous layer was extracted with ethyl acetate twice more. The combined organic layer was washed with brine, dried over sodium sulfate, and filtered. The filtrate was taken, and volatile organics were removed under reduced pressure to give a foamy brown solid. The solid was taken up in dichloromethane and purified on a silica gel column (0-20% methanol in dichloromethane) to give tert-butyl 9-((1-(3-(2,6-bis(benzyloxy)pyridin-3-yl)-5-fluoro-1-methyl-1H-indazol-6-yl)piperidin-4-yl)methyl)-3,9-diazaspiro[5.5]undecane-3-carboxylate (1.088 g) as an orange solid. LC-MS (ESI): m/z=789.5 [M+H]⁺

### Preparation of tert-butyl 9-((1-(3-(2,6-dioxopiperidin-3-yl)-5-fluoro-1-methyl-1H-indazol-6-yl)piperidin-4-yl)methyl)-3,9-diazaspiro[5.5]undecane-3-carboxylate

A solution of tert-butyl 9-((1-(3-(2,6-bis(benzyloxy)pyridin-3-yl)-5-fluoro-1-methyl-1H-indazol-6-yl)piperidin-4-yl)methyl)-3,9-diazaspiro[5.5]undecane-3-carboxylate (1.088 g, 1 equiv, 1.379 mmol) in tetrahydrofuran (30.0 mL) and N,N-dimethylformamide (6.00 mL) was treated with palladium hydroxide on carbon (193.6 mg, 20% Wt, 0.20 equiv, 275.8 μmol). The flask was evacuated and backfilled with hydrogen (3×). The mixture was stirred under hydrogen (balloon) for 21 h at 40° C. The reaction mixture was filtered through celite. The filter cake was washed with dichloromethane. The filtrate was taken, and volatile organics were removed under reduced pressure to give tert-butyl 9-((1-(3-(2,6-dioxopiperidin-3-yl)-5-fluoro-1-methyl-1H-indazol-6-yl)piperidin-4-yl)methyl)-3,9-diazaspiro[5.5]undecane-3-carboxylate (930 mg) as a dark brown solid. Carried forward without further purification. LC-MS (ESI): m/z=611.4 [M+1]⁺

### Preparation of 3-(6-(4-((3,9-diazaspiro[5.5]undecan-3-yl)methyl)piperidin-1-yl)-5-fluoro-1-methyl-1H-indazol-3-yl)piperidine-2,6-dione, 3HCl

To a solution of tert-butyl 9-((1-(3-(2,6-dioxopiperidin-3-yl)-5-fluoro-1-methyl-1H-indazol-6-yl)piperidin-4-yl)methyl)-3,9-diazaspiro[5.5]undecane-3-carboxylate (930 mg, 1 equiv, 1.52 mmol) in 1,4-dioxane (5.0 mL) was added HCl (729 mg, 5.00 mL, 4.0 M in dioxane, 13.1 equiv, 20.0 mmol). The reaction mixture was stirred at ambient temperature for 1 h. The mixture was concentrated under reduced pressure to give 3-(6-(4-((3,9-diazaspiro[5.5]undecan-3-yl)methyl)piperidin-1-yl)-5-fluoro-1-methyl-1H-indazol-3-yl)piperidine-2,6-dione, 3HCl (785 mg) as a dark brown solid which was used in the next step without further purification. LC-MS (ESI): m/z=511.4 [M+H]⁺

### Preparation of 2-((6-((5-chloro-2-(9-((1-(3-(2,6-dioxopiperidin-3-yl)-5-fluoro-1-methyl-1H-indazol-6-yl)piperidin-4-yl)methyl)-3,9-diazaspiro[5.5]undecan-3-yl)pyrimidin-4-yl)amino)-1-isopropyl-2-oxo-1,2-dihydro-1,8-naphthyridin-3-yl)oxy)-N-methylacetamide

To a solution of 2-((6-((2,5-dichloropyrimidin-4-yl)amino)-1-isopropyl-2-oxo-1,2-dihydro-1,8-naphthyridin-3-yl)oxy)-N-methylacetamide (180 mg, 1 equiv, 412 μmol) in DMSO (2.0 mL) was added 3-(6-(4-((3,9-diazaspiro[5.5]undecan-3-yl)methyl)piperidin-1-yl)-5-fluoro-1-methyl-1H-indazol-3-yl)piperidine-2,6-dione, 3HCl (383 mg, 1.5 equiv, 617 μmol) and DIEA (266 mg, 359 μL, 5.0 equiv, 2.06 mmol). The reaction mixture was stirred at 80° C. for 20 h. The reaction mixture was filtered and purified by reverse phase HPLC to give a gray solid which was taken up in acetonitrile: water, frozen, and lyophilized to afford 2-((6-((5-chloro-2-(9-((1-(3-(2,6-dioxopiperidin-3-yl)-5-fluoro-1-methyl-1H-indazol-6-yl)piperidin-4-yl)methyl)-3,9-diazaspiro[5.5]undecan-3-yl)pyrimidin-4-yl)amino)-1-isopropyl-2-oxo-1,2-dihydro-1,8-naphthyridin-3-yl)oxy)-N-methylacetamide (104 mg) as a light gray solid. 1H NMR (400 MHz, DMSO-d₆) δ (ppm)=10.84 (s, 1H), 9.02 (s, 1H), 8.68 (d, J=2.3 Hz, 1H), 8.30 (d, J=2.4 Hz, 1H), 8.07 (s, 1H), 7.95 (br d, J=4.9 Hz, 1H), 7.43 (d, J=12.5 Hz, 1H), 7.14-7.02 (m, 2H), 6.10-5.79 (m, 1H), 4.58 (s, 2H), 4.26 (dd, J=5.1, 9.6 Hz, 1H), 3.93 (s, 3H), 3.61 (br s, 4H), 3.41 (br d, J=11.4 Hz, 2H), 2.72-2.57 (m, 7H), 2.44-2.27 (m, 5H), 2.23 (br d, J=6.8 Hz, 2H), 2.18-2.08 (m, 1H), 1.81 (br d, J=11.1 Hz, 2H), 1.74-1.63 (m, 1H), 1.57 (d, J=6.9 Hz, 6H), 1.49 (br s, 4H), 1.41 (br s, 4H), 1.36-1.24 (m, 2H). LC-MS (ESI): m/z=456.4 [(M+H)/2]⁺

### Example 166. 2-((6-((5-chloro-2-((2R)-4-((1-(3-(2,6-dioxopiperidin-3-yl)-1-methyl-1H-indazol-7-yl)piperidin-4-yl)methyl)-2-(hydroxymethyl)piperazin-1-yl)pyrimidin-4-yl)amino)-1-isopropyl-2-oxo-1,2-dihydro-1,8-naphthyridin-3-yl)oxy)-N-methylacetamide (Compound 155b)

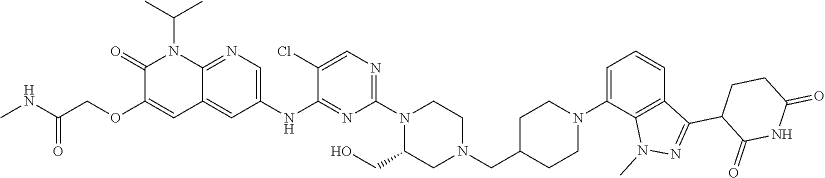 [SMILES: CNC(=O)COc1cc2cc(Nc3nc(N4CCN(CC5CCN(c6cccc7c(C8CCC(=O)NC8=O)nn(C)c67)CC5)C[C@@H]4CO)ncc3Cl)cnc2n(C(C)C)c1=O]

### Preparation of tert-butyl (R)-4-(5-chloro-4-((8-isopropyl-6-(2-(methylamino)-2-oxoethoxy)-7-oxo-7,8-dihydro-1,8-naphthyridin-3-yl)amino)pyrimidin-2-yl)-3-(hydroxymethyl)piperazine-1-carboxylate

To a stirred solution of 2-((6-((2,5-dichloropyrimidin-4-yl)amino)-1-isopropyl-2-oxo-1,2-dihydro-1,8-naphthyridin-3-yl)oxy)-N-methylacetamide (0.1 g, 0.22 mmol) and tert-butyl (R)-3-(hydroxymethyl)piperazine-1-carboxylate (0.29 g, 1.37 mmol) in DMSO (2.0 mL) was added N,N-diisopropylethylamine (0.11 mL, 0.66 mmol). The mixture was stirred at 100° C. for 16 h. The reaction mixture was poured into ice-cold water and stirred for 10 min. The precipitate obtained was filtered and dried to get crude tert-butyl (R)-4-(5-chloro-4-((8-isopropyl-6-(2-(methylamino)-2-oxoethoxy)-7-oxo-7,8-dihydro-1,8-naphthyridin-3-yl)amino)pyrimidin-2-yl)-3-(hydroxymethyl)piperazine-1-carboxylate (0.13 g) as an off white solid which was directly used for the next step without further purification.

LC-MS (ESI): m/z=617.53 [M+H]⁺

### Preparation of (R)-2-((6-((5-chloro-2-(2-(hydroxymethyl)piperazin-1-yl)pyrimidin-4-yl)amino)-1-isopropyl-2-oxo-1,2-dihydro-1,8-naphthyridin-3-yl)oxy)-N-methylacetamide

To a stirred solution of tert-butyl (R)-4-(5-chloro-4-((8-isopropyl-6-(2-(methylamino)-2-oxoethoxy)-7-oxo-7,8-dihydro-1,8-naphthyridin-3-yl)amino)pyrimidin-2-yl)-3-(hydroxymethyl)piperazine-1-carboxylate (0.13 g, 0.21 mmol)) in DCM (2.6 mL) cooled to 0° C. was added TFA (0.65 mL). The mixture was stirred at room temperature for 2 h. The reaction mixture was concentrated under reduced pressure to get a crude product which was washed with n-pentane to obtain (R)-2-((6-((5-chloro-2-(2-(hydroxymethyl)piperazin-1-yl)pyrimidin-4-yl)amino)-1-isopropyl-2-oxo-1,2-dihydro-1,8-naphthyridin-3-yl)oxy)-N-methylacetamide (0.12 g) as a brown semisolid.

LC-MS (ESI): m/z=517.25 [M+H]⁺

### Preparation of 2-((6-((5-chloro-2-((2R)-4-((1-(3-(2,6-dioxopiperidin-3-yl)-1-methyl-1H-indazol-7-yl)piperidin-4-yl)methyl)-2-(hydroxymethyl)piperazin-1-yl)pyrimidin-4-yl)amino)-1-isopropyl-2-oxo-1,2-dihydro-1,8-naphthyridin-3-yl)oxy)-N-methylacetamide

To a stirred solution of tert-butyl (R)-2-((6-((5-chloro-2-(2-(hydroxymethyl)piperazin-1-yl)pyrimidin-4-yl)amino)-1-isopropyl-2-oxo-1,2-dihydro-1,8-naphthyridin-3-yl)oxy)-N-methylacetamide (0.10 g, 0.19 mmol), 1-(3-(2,6-dioxopiperidin-3-yl)-1-methyl-1H-indazol-7-yl)piperidine-4-carbaldehyde (0.1 g, 0.28 mmol), and sodium acetate anhydrous (0.046 g, 0.57 mmol) in DCM (5.0 mL) was added acetic acid (0.10 mL). The mixture was stirred at room temperature for 3 h followed by the addition of sodium triacetoxy borohydride (0.12 g, 0.57 mmol) at 0° C. under nitrogen atmosphere. The resulting mixture was stirred at room temperature for 13 h. The reaction mixture was quenched with water (50 mL) and extracted with ethyl acetate (2×100 mL). The combined organic layers were washed with brine, dried over anhydrous Na₂SO₄, filtered and concentrated under reduced pressure to get a crude product which was purified by Prep-HPLC to afford 2-((6-((5-chloro-2-((2R)-4-((1-(3-(2,6-dioxopiperidin-3-yl)-1-methyl-1H-indazol-7-yl)piperidin-4-yl)methyl)-2-(hydroxymethyl)piperazin-1-yl)pyrimidin-4-yl)amino)-1-isopropyl-2-oxo-1,2-dihydro-1,8-naphthyridin-3-yl)oxy)-N-methylacetamide (0.039 g) as an off white solid.

LC-MS (ESI): m/z=855.48 [M+H]⁺

¹H NMR (400 MHz, DMSO-d₆): δ 10.88 (s, 1H), 9.07 (s, 1H), 8.75-8.38 (m, 2H), 8.09 (s, 1H), 7.93 (d, J=4.40 Hz, 1H), 7.38-7.21 (m, 2H), 7.01 (d, J=4.80 Hz, 1H), 5.95 (br, 1H), 4.90 (br, 1H), 4.54 (s, 3H), 4.31-4.33 (m, 1H), 4.25 (s, 3H), 3.85 (br, 1H), 3.51 (br, 1H), 3.32 (br, 2H), 3.09-3.12 (m, 2H), 2.87 (d, J=10.40 Hz, 1H), 2.50-2.51 (m, 8H), 2.36-2.37 (m, 1H), 2.31-2.32 (m, 3H), 1.89-1.92 (m, 4H), 1.73 (br, 1H), 1.57 (d, J=6.80 Hz, 6H), 1.36-1.40 (m, 3H).

### Example 167: 2-((6-((5-chloro-2-((3S,4R)-4-((4-(3-(2,6-dioxopiperidin-3-yl)-1-methyl-1H-indazol-6-yl)piperidin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide [rel-2-((6-((5-chloro-2-((3S,4R)-4-((4-(3-(2,6-dioxopiperidin-3-yl)-1-methyl-1H-indazol-6-yl)piperidin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide] (Compound 242a)

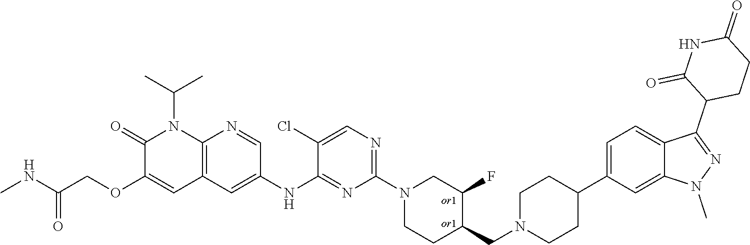 [SMILES: CNC(=O)COc1cc2cc(Nc3nc(N4CC[C@H](CN5CCC(c6ccc7c(C8CCC(=O)NC8=O)nn(C)c7c6)CC5)[C@H](F)C4)ncc3Cl)cnc2n(C(C)C)c1=O]

### Preparation of tert-butyl 4-(3-(2,6-bis(benzyloxy)pyridin-3-yl)-1-methyl-1H-indazol-6-yl)piperidine-1-carboxylate

To a stirred solution of 3-(2,6-bis(benzyloxy)pyridin-3-yl)-6-bromo-1-methyl-1H-indazole (12.0 g, 23.98 mmol) and tert-butyl 4-bromopiperidine-1-carboxylate (12.6 g, 47.96 mmol) in MeOH (180 mL) was added nickel (II) chloride ethylene glycol dimethyl ether complex (0.52 g, 2.39 mmol), 1,10-phenanthroline (0.86 g, 4.79 mmol), manganese (2.63 g, 47.96 mmol), and sodium tetrafluoroborate (1.31 g, 11.99 mmol). The mixture was stirred for 10 minutes followed by the addition of 4-ethyl pyridine (1.28 g, 11.99 mmol) at room temperature. The mixture was stirred at 70° C. for 16 h. The reaction mixture was concentrated under reduced pressure, quenched with water (1000 mL), and extracted with ethyl acetate (3×300 mL). The combined organic layer was dried over anhydrous Na₂SO₄, filtered, and concentrated under reduced pressure to obtain a crude product which was purified by flash chromatography (50-100% ethyl acetate in petroleum ether) to afford tert-butyl 4-(3-(2,6-bis(benzyloxy)pyridin-3-yl)-1-methyl-1H-indazol-6-yl)piperidine-1-carboxylate (7.0 g) as a pale yellow solid.

LC-MS (ESI): m/z=605.36 [M+H]⁺

### Preparation of 6-(benzyloxy)-3-(1-methyl-6-(piperidin-4-yl)-1H-indazol-3-yl)pyridin-2-ol

To a solution of tert-butyl 4-(3-(2,6-bis(benzyloxy)pyridin-3-yl)-1-methyl-1H-indazol-6-yl)piperidine-1-carboxylate (7.0 g, 11.58 mmol) in DCM (210 mL) was added TFA (14 mL, 2 V) at 0° C. The mixture was stirred at room temperature for 2 h. The reaction mixture was concentrated under reduced pressure and co-distilled with DCM (2×50 mL) to obtain a crude product which was triturated with diethyl ether (2×10 mL) to afford 6-(benzyloxy)-3-(1-methyl-6-(piperidin-4-yl)-1H-indazol-3-yl)pyridin-2-ol (8.0 g) as a pale brown solid.

LC-MS (ESI): m/z=415.36 [M+H]⁺

### Preparation of cis-tert-butyl 4-(4-(3-(6-(benzyloxy)-2-hydroxypyridin-3-yl)-1-methyl-1H-indazol-6-yl)piperidine-1-carbonyl)-3-fluoropiperidine-1-carboxylate

To a stirred solution of 6-(benzyloxy)-3-(1-methyl-6-(piperidin-4-yl)-1H-indazol-3-yl)pyridin-2-ol (4.0 g, 9.65 mmol) and cis-1-(tert-butoxycarbonyl)-3-fluoropiperidine-4-carboxylic acid (2.38 g, 9.65 mmol) in DMF (40 mL, 10 v) was added DIPEA (3.11 g, 24.12 mmol) and HATU (4.4 g, 11.58 mmol). The mixture was stirred at room temperature for 12 h. The reaction mixture was cooled to room temperature and treated with ice water. The solid precipitate was filtered and dried to get a crude product which was purified by flash chromatography (50-100% ethyl acetate in petroleum ether) to afford cis-tert-butyl 4-(4-(3-(6-(benzyloxy)-2-hydroxypyridin-3-yl)-1-methyl-1H-indazol-6-yl)piperidine-1-carbonyl)-3-fluoropiperidine-1-carboxylate (4.0 g) as a pale yellow solid.

LC-MS (ESI): m/z=644.36 [M+H]⁺

### Preparation of cis-tert-butyl 4-((4-(3-(6-(benzyloxy)-2-hydroxypyridin-3-yl)-1-methyl-1H-indazol-6-yl)piperidin-1-yl)methyl)-3-fluoropiperidine-1-carboxylate

To a stirred solution of cis-tert-butyl 4-(4-(3-(6-(benzyloxy)-2-hydroxypyridin-3-yl)-1-methyl-1H-indazol-6-yl)piperidine-1-carbonyl)-3-fluoropiperidine-1-carboxylate (4.0 g, 6.21 mmol) in THF (80.0 mL) was added BH₃.DMS (neat) (8.0 mL) at 0° C. The mixture was stirred at room temperature for 16 h. The reaction mixture was quenched with MeOH (30 mL), concentrated, and co-distilled with methanol (30 mL) to get a crude product. To this mixture was added MeOH (150 mL). The resulting mixture was refluxed for 4 h, cooled to room temperature, concentrated, and dried to afford cis-tert-butyl 4-((4-(3-(6-(benzyloxy)-2-hydroxypyridin-3-yl)-1-methyl-1H-indazol-6-yl)piperidin-1-yl)methyl)-3-fluoropiperidine-1-carboxylate (2.0 g) as an off white solid.

LC-MS (ESI): m/z=630.39 [M+H]⁺

### Preparation of tert-butyl (3S,4R)-4-((4-(3-(2-(benzyloxy)-6-hydroxypyridin-3-yl)-1-methyl-1H-indazol-6-yl)piperidin-1-yl)methyl)-3-fluoropiperidine-1-carboxylate and tert-butyl (3R,4S)-4-((4-(3-(2-(benzyloxy)-6-hydroxypyridin-3-yl)-1-methyl-1H-indazol-6-yl)piperidin-1-yl)methyl)-3-fluoropiperidine-1-carboxylate

Stereoisomers of cis-tert-butyl 4-((4-(3-(6-(benzyloxy)-2-hydroxypyridin-3-yl)-1-methyl-1H-indazol-6-yl)piperidin-1-yl)methyl)-3-fluoropiperidine-1-carboxylate (1.50 g) were separated by chiral SFC to afford tert-butyl (3S,4R)-4-((4-(3-(2-(benzyloxy)-6-hydroxypyridin-3-yl)-1-methyl-1H-indazol-6-yl)piperidin-1-yl)methyl)-3-fluoropiperidine-1-carboxylate as an off white solid (0.60 g) and tert-butyl (3R,4S)-4-((4-(3-(2-(benzyloxy)-6-hydroxypyridin-3-yl)-1-methyl-1H-indazol-6-yl)piperidin-1-yl)methyl)-3-fluoropiperidine-1-carboxylate as an off white solid (0.60 g).

LC-MS (ESI): m/z=630.39 [M+H]⁺

LC-MS (ESI): m/z=630.70 [M+H]⁺

### Preparation of tert-butyl (3S,4R)-4-((4-(3-(2,6-dioxopiperidin-3-yl)-1-methyl-1H-indazol-6-yl)piperidin-1-yl)methyl)-3-fluoropiperidine-1-carboxylate

To a stirred solution of tert-butyl (3S,4R)-4-((4-(3-(2-(benzyloxy)-6-hydroxypyridin-3-yl)-1-methyl-1H-indazol-6-yl)piperidin-1-yl)methyl)-3-fluoropiperidine-1-carboxylate (0.60 g, 0.95 mmol) in THF (60.0 mL) was added 20% palladium hydroxide (0.72 g, 120% w/w). The mixture was stirred under H₂ (80 psi) pressure for 16 h at room temperature. The reaction mixture was diluted with THF (80 mL), filtered through a celite bed, and washed with THF:DCM (1:1, 200 mL). The filtrate was concentrated and dried to obtain a crude product which was triturated with diethyl ether, and dried to afford tert-butyl (3S,4R)-4-((4-(3-(2,6-dioxopiperidin-3-yl)-1-methyl-1H-indazol-6-yl)piperidin-1-yl)methyl)-3-fluoropiperidine-1-carboxylate (0.40 g) as a light brown semi solid.

LC-MS (ESI): m/z=542.51 [M+H]⁺

### Preparation of 3-(6-(1-(((3S,4R)-3-fluoropiperidin-4-yl)methyl)piperidin-4-yl)-1-methyl-1H-indazol-3-yl)piperidine-2,6-dione

To a stirred solution of tert-butyl (3S,4R)-4-((4-(3-(2,6-dioxopiperidin-3-yl)-1-methyl-1H-indazol-6-yl)piperidin-1-yl)methyl)-3-fluoropiperidine-1-carboxylate (0.40 g, 0.74 mmol) in DCM (12.0 mL) was added TFA (2.0 mL) at 0° C. The mixture was stirred at room temperature for 2 h. The reaction mixture was concentrated under reduced pressure and co-distilled with DCM (2×20 mL) to obtain a crude product which was triturated with diethyl ether (2×10 mL), and dried to afford 3-(6-(1-(((3S,4R)-3-fluoropiperidin-4-yl)methyl)piperidin-4-yl)-1-methyl-1H-indazol-3-yl)piperidine-2,6-dione (0.40 g) as a pale brown solid.

LC-MS (ESI): m/z=442.26 [M+H]⁺

### Preparation of 2-((6-((5-chloro-2-((3S,4R)-4-((4-(3-(2,6-dioxopiperidin-3-yl)-1-methyl-1H-indazol-6-yl)piperidin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide [rel-2-((6-((5-chloro-2-((3S,4R)-4-((4-(3-(2,6-dioxopiperidin-3-yl)-1-methyl-1H-indazol-6-yl)piperidin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide]

To a stirred solution of 3-(6-(1-(((3S,4R)-3-fluoropiperidin-4-yl)methyl)piperidin-4-yl)-1-methyl-1H-indazol-3-yl)piperidine-2,6-dione (0.20 g, 0.45 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.08 g, 0.18 mmol) in DMSO (2.0 mL) was added DIPEA (2.0 mL). The mixture was stirred at 100° C. for 6 h. The reaction mixture was cooled to room temperature and treated with ice water. The precipitated solid was filtered and dried to obtain crude product which was purified by prep-HPLC to afford 2-((6-((5-chloro-2-((3S,4R)-4-((4-(3-(2,6-dioxopiperidin-3-yl)-1-methyl-1H-indazol-6-yl)piperidin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide [rel-2-((6-((5-chloro-2-((3S,4R)-4-((4-(3-(2,6-dioxopiperidin-3-yl)-1-methyl-1H-indazol-6-yl)piperidin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide] (0.023 g) as an off white solid.

LC-MS (ESI): m/z=842.52 [M+H]⁺

¹H NMR (400 MHz, DMSO-d₆): δ 10.87 (s, 1H), 9.06 (s, 1H), 8.68 (d, J=2.4 Hz, 1H), 8.30 (d, J=2.0 Hz, 1H), 8.07 (s, 1H), 7.95 (d, J=4.4 Hz, 1H), 7.61 (d, J=8.4 Hz, 1H), 7.42 (s, 1H), 7.12 (s, 1H), 7.05 (d, J=8.4 Hz, 1H), 5.95 (br s, 1H), 4.95-4.8 (m, 2H), 4.59 (s, 3H), 4.35-4.29 (m, 1H), 3.95 (s, 3H), 3.1-2.92 (m, 5H), 2.71-2.60 (m, 5H), 2.40-2.30 (m, 2H), 2.25-2.12 (m, 2H), 2.10-2.01 (m, 3H), 1.79 (br s, 4H), 1.60-1.51 (m, 7H), 1.45-1.35 (m, 1H).

### Example 168: 2-((6-((5-chloro-2-((3R,4S)-4-((4-(3-(2,6-dioxopiperidin-3-yl)-1-methyl-1H-indazol-6-yl)piperidin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide [rel-2-((6-((5-chloro-2-((3R,4S)-4-((4-(3-(2,6-dioxopiperidin-3-yl)-1-methyl-1H-indazol-6-yl)piperidin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide] (Compound 242b)

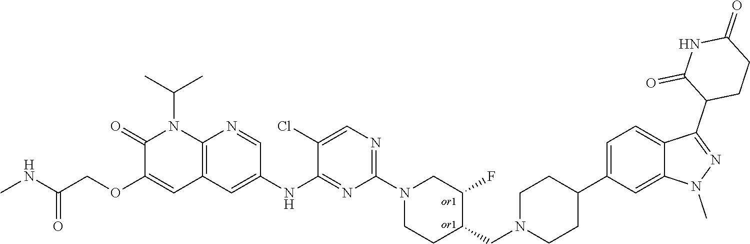 [SMILES: CNC(=O)COc1cc2cc(Nc3nc(N4CC[C@@H](CN5CCC(c6ccc7c(C8CCC(=O)NC8=O)nn(C)c7c6)CC5)[C@@H](F)C4)ncc3Cl)cnc2n(C(C)C)c1=O]

### Preparation of tert-butyl (3R,4S)-4-((4-(3-(2,6-dioxopiperidin-3-yl)-1-methyl-1H-indazol-6-yl)piperidin-1-yl)methyl)-3-fluoropiperidine-1-carboxylate

To a stirred solution of tert-butyl (3R,4S)-4-((4-(3-(2-(benzyloxy)-6-hydroxypyridin-3-yl)-1-methyl-1H-indazol-6-yl)piperidin-1-yl)methyl)-3-fluoropiperidine-1-carboxylate (0.60 g, 0.95 mmol) in THF (60.0 mL) was added 20% palladium hydroxide (0.72 g, 120% w/w). The mixture was stirred under H₂ (80 psi) pressure for 16 h at room temperature. The reaction mixture was diluted with THF (80 mL), filtered through a celite bed, and washed with THF:DCM (1:1, 200 mL). The filtrate was concentrated and dried to obtain a crude product which was triturated with diethyl ether and dried to afford tert-butyl (3R,4S)-4-((4-(3-(2,6-dioxopiperidin-3-yl)-1-methyl-1H-indazol-6-yl)piperidin-1-yl)methyl)-3-fluoropiperidine-1-carboxylate (0.40 g) as a light brown semi-solid.

LC-MS (ESI): m/z=540.37 [M−H]

### Preparation of 3-(6-(1-(((3R,4S)-3-fluoropiperidin-4-yl)methyl)piperidin-4-yl)-1-methyl-1H-indazol-3-yl)piperidine-2,6-dione

To a stirred solution of tert-butyl (3R,4S)-4-((4-(3-(2,6-dioxopiperidin-3-yl)-1-methyl-1H-indazol-6-yl)piperidin-1-yl)methyl)-3-fluoropiperidine-1-carboxylate (0.40 g, 0.73 mmol) in DCM (12.0 mL) was added TFA (2.0 mL) at 0° C. The mixture was stirred at room temperature for 2 h. the reaction mixture was concentrated under reduced pressure and co-distilled with DCM (2×20 mL) to obtain a crude product which was triturated with diethyl ether (2×10 mL), and dried to afford 3-(6-(1-(((3R,4S)-3-fluoropiperidin-4-yl)methyl)piperidin-4-yl)-1-methyl-1H-indazol-3-yl)piperidine-2,6-dione (0.28 g) as a pale brown solid.

LC-MS (ESI): m/z=442.26 [M+H]⁺

### Preparation of 2-((6-((5-chloro-2-((3R,4S)-4-((4-(3-(2,6-dioxopiperidin-3-yl)-1-methyl-1H-indazol-6-yl)piperidin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide [rel-2-((6-((5-chloro-2-((3R,4S)-4-((4-(3-(2,6-dioxopiperidin-3-yl)-1-methyl-1H-indazol-6-yl)piperidin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide]

To a stirred solution of 3-(6-(1-(((3R,4S)-3-fluoropiperidin-4-yl)methyl)piperidin-4-yl)-1-methyl-1H-indazol-3-yl)piperidine-2,6-dione (0.13 g, 0.29 mmol) and 2-((6-((2,5-dichloropyrimidin-4-yl)amino)-1-isopropyl-2-oxo-1,2-dihydro-1,8-naphthyridin-3-yl)oxy)-N-methylacetamide (0.04 g, 0.11 mmol) in DMSO (1.3 mL) was added DIPEA (1.3 mL). The mixture was stirred at 100° C. for 6 h. The reaction mixture was cooled to room temperature and poured into ice water. The precipitated solid was filtered and dried to obtain a crude product which was purified by prep-HPLC to afford 2-((6-((5-chloro-2-((3R,4S)-4-((4-(3-(2,6-dioxopiperidin-3-yl)-1-methyl-1H-indazol-6-yl)piperidin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide [rel-2-((6-((5-chloro-2-((3R,4S)-4-((4-(3-(2,6-dioxopiperidin-3-yl)-1-methyl-1H-indazol-6-yl)piperidin-1-yl)methyl)-3-fluoropiperidin-1-yl)pyrimidin-4-yl)amino)-1-isopropyl-2-oxo-1,2-dihydro-1,8-naphthyridin-3-yl)oxy)-N-methylacetamide](0.008 g) as an off white solid.

LC-MS (ESI): m/z=842.52 [M+H]⁺

¹HNMR (400 MHz, DMSO-d₆): δ 10.87 (s, 1H), 9.06 (s, 1H), 8.68 (d, J=2.4 Hz, 1H), 8.30 (d, J=2.0 Hz, 1H), 8.07 (s, 1H), 7.95 (d, J=4.4 Hz, 1H), 7.61 (d, J=8.4 Hz, 1H), 7.42 (s, 1H), 7.12 (s, 1H), 7.05 (d, J=8.4 Hz, 1H), 5.95 (br s, 1H), 4.95-4.81 (m, 2H), 4.59 (s, 3H), 4.35-4.29 (m, 1H), 3.95 (s, 3H), 3.10-2.92 (m, 5H), 2.71-2.60 (m, 5H), 2.40-2.30 (m, 2H), 2.25-2.12 (m, 2H), 2.10-2.01 (m, 3H), 1.79 (br s, 4H), 1.60-1.51 (m, 7H), 1.45-1.35 (m, 1H)

### Example 169: 2-((6-((5-chloro-2-(4-((1-(3-(2,6-dioxopiperidin-3-yl)-1-methyl-1H-indazol-7-yl)-3,3-difluoropiperidin-4-yl)methyl)piperazin-1-yl)pyrimidin-4-yl)amino)-1-isopropyl-2-oxo-1,2-dihydro-1,8-naphthyridin-3-yl)oxy)-N-methylacetamide (Compound 241)

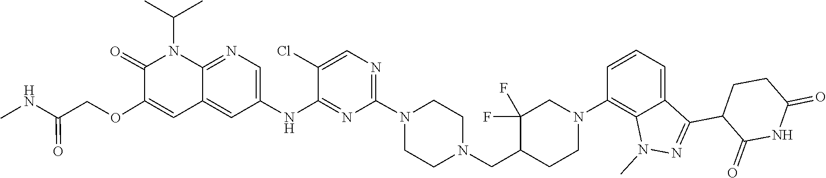 [SMILES: CNC(=O)COc1cc2cc(Nc3nc(N4CCN(CC5CCN(c6cccc7c(C8CCC(=O)NC8=O)nn(C)c67)CC5(F)F)CC4)ncc3Cl)cnc2n(C(C)C)c1=O]

### Preparation of tert-butyl 3,3-difluoro-4-((((4-nitrophenyl) sulfonyl)oxy) methyl)piperidine-1-carboxylate

To stirred solution of tert-butyl 3,3-difluoro-4-(hydroxymethyl)piperidine-1-carboxylate (1.0 g, 3.98 mmol) and 4-nitrobenzenesulfonyl chloride (1.76 g, 7.96 mmol) in CH₃CN (20.0 mL) was added triethylamine (1.6 mL) and DMAP (0.048 g, 3.98 mmol) at 0° C. The mixture was stirred for 2 h. The reaction mixture was quenched with water (20 mL) and extracted with ethyl acetate (50 mL). The organic layer was separated, washed with brine (20 mL), dried over anhydrous Na₂SO₄, filtered, and concentrated to get a crude product which was purified by flash column chromatography (10% ethyl acetate in petroleum ether) to afford tert-butyl 3,3-difluoro-4-((((4-nitrophenyl) sulfonyl)oxy)methyl)piperidine-1-carboxylate (0.7 g) as an off white solid.

LC-MS (ESI): m/z=459.10 [M+Na]⁺

### Preparation of (3,3-difluoropiperidin-4-yl)methyl 4-nitrobenzenesulfonate

To a stirred solution of tert-butyl 3,3-difluoro-4-((((4-nitrophenyl) sulfonyl)oxy) methyl)piperidine-1-carboxylate (1.0 g, 1.60 mmol) in DCM (7.0 mL) was added TFA (0.7 mL) at 0° C. The reaction mixture was stirred at room temperature for 3 h. The reaction mixture was concentrated under reduced pressure to get (3,3-difluoropiperidin-4-yl)methyl 4-nitrobenzenesulfonate (0.7 g) as a brown solid.

LC-MS (ESI): m/z=337.06 [M+H]⁺

### Preparation of benzyl 3,3-difluoro-4-((((4-nitrophenyl) sulfonyl)oxy)methyl)piperidine-1-carboxylate

To stirred solution of (3,3-difluoropiperidin-4-yl)methyl 4-nitrobenzenesulfonate (0.7 g, 2.08 mmol) in THF (10.0 mL) at 0° C. was added triethylamine (1.08 mL, 0.20 mmol) and DMAP (0.021 g, 6.24 mmol). The resulting mixture was stirred at room temperature for 12 h. The reaction mixture was quenched with water (20 mL) and extracted with ethyl acetate (50 mL). The organic layer was separated, washed with brine (20 mL), dried over anhydrous Na₂SO₄, filtered, and concentrated to get crude product which was purified by flash column chromatography (30% ethyl acetate in petroleum ether) to afford benzyl 3,3-difluoro-4-((((4-nitrophenyl) sulfonyl)oxy)methyl)piperidine-1-carboxylate (0.8 g) as a brown gum.

LC-MS (ESI): m/z=493.30 [M+Na]⁺

### Preparation of tert-butyl 4-((1-((benzyloxy) carbonyl)-3,3-difluoropiperidin-4-yl)methyl)piperazine-1-carboxylate

To a stirred solution of benzyl 3,3-difluoro-4-((((4-nitrophenyl) sulfonyl)oxy)methyl)piperidine-1-carboxylate (0.8 g, 1.11 mmol) was added Boc-piperazine (0.64 g, 3.33 mmol) and DIPEA (1.39 mL, 7.7 mmol) in acetonitrile (8.0 mL) at rt. The resultant mixture was stirred at 80° C. for 12 h. The reaction mixture was cooled to room temperature, quenched with water (20 mL), and extracted with ethyl acetate (50 mL). The organic layer was separated, washed with brine (20 mL), dried over anhydrous Na₂SO₄, filtered, and concentrated to get a crude product which was purified by flash column chromatography (40% ethyl acetate in petroleum ether) to afford tert-butyl 4-((1-((benzyloxy) carbonyl)-3,3-difluoropiperidin-4-yl)methyl)piperazine-1-carboxylate (0.5 g) as colorless gum.

LC-MS (ESI): m/z=454.27 [M+H]⁺

### Preparation of tert-butyl 4-((3,3-difluoropiperidin-4-yl)methyl)piperazine-1-carboxylate

To a stirred solution of tert-butyl 4-((1-((benzyloxy) carbonyl)-3,3-difluoropiperidin-4-yl)methyl)piperazine-1-carboxylate (0.5 g, 1.10 mmol) in THF (5.0 ml) was added 10% Pd/C (100% w/w) (0.5 g). The reaction mixture was kept under hydrogen pressure (90 psi) at room temperature for 12 h. The reaction mixture was filtered through celite, and the filtrate was concentrated under reduced pressure to get tert-butyl 4-((3,3-difluoropiperidin-4-yl)methyl)piperazine-1-carboxylate (0.4 g) as a brown solid.

LC-MS (ESI): m/z=320.37 [M+H]⁺

### Preparation of tert-butyl 4-((1-(3-(2,6-bis(benzyloxy)pyridin-3-yl)-1-methyl-1H-indazol-7-yl)-3,3-difluoropiperidin-4-yl)methyl)piperazine-1-carboxylate

A stirred solution of tert-butyl 4-((3,3-difluoropiperidin-4-yl)methyl)piperazine-1-carboxylate (0.4 g, 1.25 mmol) and 3-(2,6-bis(benzyloxy)pyridin-3-yl)-7-bromo-1-methyl-1H-indazole in 1,4-dioxane (16.0 mL) was degassed using nitrogen for 15 min. To this mixture was added Pd-PEPPSI-iHEPT (C₁) (0.08 g, 0.08 mmol) and NaOtBu (0.36 g, 3.75 mmol). The resulting mixture was stirred at 80° C. for 6 h. The reaction mixture was filtered through celite, and the filtrate was concentrated under reduced pressure to get a crude product which was purified by flash column chromatography (60-70% ethyl acetate in petroleum ether) to provide tert-butyl 4-((1-(3-(2,6-bis(benzyloxy)pyridin-3-yl)-1-methyl-1H-indazol-7-yl)-3,3-difluoropiperidin-4-yl)methyl)piperazine-1-carboxylate (0.1 g) as an off-white solid

LC-MS (ESI): m/z=739.68 [M+H]⁺

### Preparation of tert-butyl 4-((1-(3-(2,6-dioxopiperidin-3-yl)-1-methyl-1H-indazol-7-yl)-3,3-difluoropiperidin-4-yl)methyl)piperazine-1-carboxylate

To a stirred solution of tert-butyl 4-((1-(3-(2,6-bis(benzyloxy)pyridin-3-yl)-1-methyl-1H-indazol-7-yl)-3,3-difluoropiperidin-4-yl)methyl)piperazine-1-carboxylate (0.1 g, 0. mmol) in THF (2.0 mL) was added 20% Pd(OH)₂ (100% w/w) (0.1 g). The reaction mixture was kept under hydrogen pressure (90 psi) at room temperature for 12 h. The reaction mixture was filtered through celite, and the filtrate was concentrated under reduced pressure to get tert-butyl 4-((1-(3-(2,6-dioxopiperidin-3-yl)-1-methyl-1H-indazol-7-yl)-3,3-difluoropiperidin-4-yl)methyl)piperazine-1-carboxylate (0.04 g) as a brown gum

LC-MS (ESI): m/z=561.68 [M+H]⁺

### Preparation of 3-(7-(3,3-difluoro-4-(piperazin-1-ylmethyl)piperidin-1-yl)-1-methyl-1H-indazol-3-yl)piperidine-2,6-dione

To a stirred solution of tert-butyl 4-((1-(3-(2,6-dioxopiperidin-3-yl)-1-methyl-1H-indazol-7-yl)-3,3-difluoropiperidin-4-yl)methyl)piperazine-1-carboxylate (0.04 g, 0.07 mmol) in DCM (2.0 ml) was added TFA (2.0 ml) at 0° C. The mixture was stirred at room temperature for 3 h. The reaction mixture was concentrated under reduced pressure to afford 3-(7-(3,3-difluoro-4-(piperazin-1-ylmethyl)piperidin-1-yl)-1-methyl-1H-indazol-3-yl)piperidine-2,6-dione (0.02 g) as a brown solid.

LC-MS (ESI): m/z=477.24 [M+H]⁺

### Preparation of 2-((6-((5-chloro-2-(4-((1-(3-(2,6-dioxopiperidin-3-yl)-1-methyl-1H-indazol-7-yl)-3,3-difluoropiperidin-4-yl)methyl)piperazin-1-yl)pyrimidin-4-yl)amino)-1-isopropyl-2-oxo-1,2-dihydro-1,8-naphthyridin-3-yl)oxy)-N-methylacetamide

To a stirred solution of 3-(7-(3,3-difluoro-4-(piperazin-1-ylmethyl)piperidin-1-yl)-1-methyl-1H-indazol-3-yl)piperidine-2,6-dione (0.02 g, 0.04 mmol) in dry DMSO (1.0 mL) was added N,N-diisopropylethylamine (0.5 mL) and 2-((6-((2,5-dichloropyrimidin-4-yl)amino)-1-isopropyl-2-oxo-1,2-dihydro-1,8-naphthyridin-3-yl)oxy)-N-methylacetamide (0.009 g, 0.02 mmol) at room temperature. The mixture was heated at 120° C. and stirred for 4 h. The reaction mixture was cooled to room temperature, and the solvent was removed under reduced pressure to get a crude product which was purified by prep-HPLC to afford 2-((6-((5-chloro-2-(4-((1-(3-(2,6-dioxopiperidin-3-yl)-1-methyl-1H-indazol-7-yl)-3,3-difluoropiperidin-4-yl)methyl)piperazin-1-yl)pyrimidin-4-yl)amino)-1-isopropyl-2-oxo-1,2-dihydro-1,8-naphthyridin-3-yl)oxy)-N-methylacetamide (0.009 g) as an off white solid.

LC-MS (ESI): m/z=861.45 [M+H]⁺

¹H NMR data: (400 MHz, DMSO-d₆): δ 10.88 (s, 1H), 9.09 (s, 1H), 8.67 (d, J=2.40 Hz, 1H), 8.32 (d, J=2.40 Hz, 1H), 8.10 (s, 1H), 7.95 (d, J=4.40 Hz, 1H), 7.46 (d, J=7.60 Hz, 1H), 7.05-7.03 (m, 3H), 5.96 (br s, 1H), 4.59 (s, 2H), 4.35-4.31 (m, 1H), 4.23 (s, 3H), 3.63 (s, 4H), 3.41-3.32 (m, 1H), 3.29-3.21 (m, 2H), 2.68-2.61 (m, 6H), 2.63-2.56 (m, 4H), 2.35-2.31 (m, 3H), 2.16-2.09 (m, 2H), 1.65 (d, J=10.80 Hz, 8H).

The following examples were synthesized using methods similar to those described in the preceding examples:

| Example | Comp | LCMS | |
|---|---|---|---|
| No. | No. | (ESI) | ¹H NMR |
| 81 | 163a | m/z = | ¹H NMR (400 MHz, DMSO-d6): δ 10.87 (s, 1H), 9.09 (s, 1H), 8.67 (d, |
| | | 447.3 | J = 2.5 Hz, 1H), 8.30 (s, 1H), 8.11 (s, 1H), 7.94 (d, J = 4.9 Hz, 1H), |
| | | [M/2 + H]⁺ | 7.41-7.30 (m, 1H), 7.08 (s, 1H), 7.01 (d, J = 4.7 Hz, 2H), 5.95 (s, 1H), |
| | | | 4.57 (s, 2H), 4.48-4.36 (m, 1H), 4.33 (dd, J = 9.5, 5.1 Hz, 1H), 4.24 |
| | | | (s, 3H), 4.09-3.89 (m, 1H), 3.65-3.48 (m, 2H), 3.30-3.13 (m, 4H), |
| | | | 3.00-2.90 (m, 1H), 2.85-2.76 (m, 1H), 2.73-2.66 (m, 4H), 2.66- |
| | | | 2.59 (m, 2H), 2.38-2.26 (m, 2H), 2.16 (dd, J = 13.1, 5.9 Hz, 1H), 1.95- |
| | | | 1.80 (m, 2H), 1.80-1.65 (m, 2H), 1.57 (d, J = 6.9 Hz, 6H), 1.44- |
| | | | 1.29 (m, 2H). |
| 82 | 164 | m/z = | ¹H NMR (400 MHz, DMSO-d6): δ 10.85 (s, 1H), 9.07 (s, 1H), 8.68 (d, |
| | | 743.3 | J = 2.5 Hz, 1H), 8.30 (d, J = 2.6 Hz, 1H), 8.10 (s, 1H), 7.90 (d, J = 4.9 |
| | | [M + H]⁺ | Hz, 1H), 7.56 (d, J = 8.8 Hz, 1H), 7.14 (d, J = 2.0 Hz, 1H), 7.09 (s, 1H), |
| | | | 6.76 (dd, J = 8.8, 2.0 Hz, 1H), 5.94 (s, 1H), 4.82-4.70 (m, 1H), 4.55 |
| | | | (s, 2H), 4.29 (dd, J = 9.4, 5.1 Hz, 1H), 4.14-3.99 (m, 2H), 3.92 (s, |
| | | | 3H), 3.51 (t, J = 10.3 Hz, 2H), 2.71-2.61 (m, 2H), 2.59 (d, J = 4.7 Hz, |
| | | | 3H), 2.38-2.25 (m, 1H), 2.20-2.11 (m, 1H), 2.07-1.96 (m, 2H), |
| | | | 1.71-1.60 (m, 2H), 1.55 (d, J = 6.9 Hz, 6H). |
| 83 | 165 | m/z = | ¹H NMR (400 MHz, DMSO-d6): δ 10.84 (s, 1H), 9.13 (s, 1H), 8.69 (d, |
| | | 728.3 | J = 2.5 Hz, 1H), 8.38 (d, J = 2.5 Hz, 1H), 8.14 (s, 1H), 8.03-7.84 (m, |
| | | [M + H]⁺ | 1H), 7.52 (d, J = 8.9 Hz, 1H), 7.15 (s, 1H), 6.99 (d, J = 9.1 Hz, 1H), |
| | | | 6.94 (s, 1H), 5.98 (s, 1H), 4.61 (s, 2H), 4.26 (dd, J = 9.2, 5.1 Hz, 1H), |
| | | | 3.90 (s, 3H), 3.86-3.72 (m, 4H), 3.30-3.24 (m, 4H), 2.67 (d, J = 4.6 |
| | | | Hz, 4H), 2.64-2.58 (m, 1H), 2.32-2.25 (m, 1H), 2.20-2.10 (m, 1H), |
| | | | 1.58 (d, J = 6.9 Hz, 6H). |
| 84 | 166 | m/z = | ¹H NMR (400 MHz, DMSO-d6): δ 10.87 (s, 1H), 9.12 (s, 1H), 8.68 (s, |
| | | 728.3 | 1H), 8.34 (s, 1H), 8.20-8.09 (m, 1H), 7.89 (s, 1H), 7.41 (d, J = 7.6 Hz, |
| | | [M + H]⁺ | 1H), 7.14 (s, 1H), 7.11-6.91 (m, 2H), 5.95 (s, 1H), 4.55 (s, 2H), 4.40- |
| | | | 4.32 (m, 1H), 4.29 (s, 3H), 3.31-3.27 (m, 2H), 3.26-3.13 (m, 4H), |
| | | | 2.88-2.74 (m, 2H), 2.73-2.60 (m, 2H), 2.60-2.55 (m, 3H), 2.40- |
| | | | 2.25 (m, 1H), 2.25-2.08 (m, 1H), 1.55 (d, J = 6.8 Hz, 6H). |
| 85 | 167 | m/z = | ¹H NMR (400 MHz, DMSO-d6): δ 10.87 (s, 1H), 9.07 (s, 1H), 8.67 (d, |
| | | 842.4 | J = 2.5 Hz, 1H), 8.31 (d, J = 2.5 Hz, 1H), 8.10 (s, 1H), 7.94 (q, J = 4.7 |
| | | [M + H]⁺ | Hz, 1H), 7.59 (d, J = 8.4 Hz, 1H), 7.42 (s, 1H), 7.10 (s, 1H), 7.02 (dd, J = |
| | | | 8.5, 1.3 Hz, 1H), 5.96 (s, 1H), 4.58 (s, 2H), 4.32 (dd, J = 9.7, 5.1 Hz, |
| | | | 1H), 4.22 (d, J = 12.9 Hz, 2H), 3.96 (s, 3H), 3.22 (t, J = 11.8 Hz, 2H), |
| | | | 3.02 (d, J = 11.2 Hz, 2H), 2.67 (d, J = 4.7 Hz, 3H), 2.65-2.56 (m, |
| | | | 2H), 2.34 (dd, J = 9.3, 4.5 Hz, 1H), 2.28-2.10 (m, 3H), 1.88 (t, J = |
| | | | 11.8 Hz, 2H), 1.81-1.67 (m, 4H), 1.57 (d, J = 6.9 Hz, 6H). |
| 86 | 168a | m/z = | ¹H NMR (400 MHz, DMSO-d6): δ 10.88 (s, 1H), 9.07 (s, 1H), 8.65 (d, |
| | | 839.52 | J = 2.40 Hz, 1H), 8.45 (s, 1H), 8.36 (d, J = 2.40 Hz, 1H), 8.10 (s, 1H), |
| | | [M + H]⁺ | 7.97 (d, J = 4.40 Hz, 1H), 7.36-7.36 (m, 1H), 7.01-7.00 (m, 2H), 5.09 |
| | | | (br s, 1H), 4.61 (s, 3H), 4.33-4.31 (m, 1H), 4.24 (s, 4H), 3.20-3.10 (m, |
| | | | 3H), 2.68 (s, 4H), 2.67-2.66 (m, 4H), 2.21-2.19 (m, 4H), 2.07-2.05 (m, |
| | | | 1H), 1.87-1.84 (m, 3H), 1.62 (br s, 1H), 1.58 (d, J = 6.80 Hz, 6H), |
| | | | 1.39-1.36 (m, 3H), 1.22 (d, J = 6.80 Hz, 3H). |
| 87 | 168b | m/z = | ¹H NMR (400 MHz, DMSO-d6): δ 10.88 (s, 1H), 9.07 (s, 1H), 8.65 (d, |
| | | 839.52 | J = 2.40 Hz, 1H), 8.36 (d, J = 2.00 Hz, 1H), 8.10 (s, 1H), 7.97 (d, J = |
| | | [M + H]⁺ | 4.40 Hz, 1H), 7.37-7.36 (m, 1H), 7.05-7.00 (m, 3H), 5.96 (br s, 1H), |
| | | | 4.59-4.55 (m, 3H), 4.35-4.25 (m, 5H), 3.24 (br, 3H), 2.91-2.87 (m, 1H), |
| | | | 2.80-2.76 (m, 1H), 2.70-2.65 (m, 7H), 2.33-2.30 (m, 4H), 2.07-2.01 (m, |
| | | | 1H), 1.99-1.86 (m, 3H), 1.79-1.70 (m, 1H), 1.57 (d, J = 6.80 Hz, 6H), |
| | | | 1.50-1.34 (m, 2H), 1.22 (d, J = 6.40 Hz, 3H). |
| 88 | 170 | m/z = | ¹H NMR (400 MHz, DMSO-d6): δ 10.87 (s, 1H), 9.14 (s, 1H), 8.66 (d, |
| | | 837.58 | J = 2.00 Hz, 1H), 8.38 (d, J = 2.00 Hz, 2H), 7.98 (d, J = 4.40 Hz, 1H), |
| | | [M + H]⁺ | 7.36-7.35 (m, 1H), 7.27 (s, 1H), 7.01-7.00 (m, 2H), 5.31 (q, J = 9.20 |
| | | | Hz, 2H), 4.65 (s, 2H), 4.33-4.28 (m, 1H), 4.24 (s, 3H), 3.61 (s, 4H), |
| | | | 3.25-3.23 (m, 3H), 2.62-2.59 (m, 6H), 2.40-2.39 (m, 4H), 2.30-2.27 (m, |
| | | | 3H), 2.18-2.14 (m, 1H), 1.90-1.87 (m, 2H), 1.73 (br s, 1H), 1.50-1.44 |
| | | | (m, 2H). |
| 89 | 171 | m/z = | ¹H NMR (400 MHz, DMSO-d6): δ 10.90 (s, 1H), 9.01 (s, 1H), 8.70 (d, |
| | | 853.61 | J = 2.40 Hz, 1H), 8.27 (d, J = 2.00 Hz, 1H), 8.07 (s, 1H), 7.93 (d, J = |
| | | [M + H]⁺ | 4.40 Hz, 1H), 7.49 (d, J = 9.20 Hz, 1H), 7.11 (s, 1H), 6.91 (d, J = 9.20 |
| | | | Hz, 1H), 6.82 (s, 1H), 5.95 (s, 1H), 4.58 (s, 2H), 4.27-4.24 (m, 1H), |
| | | | 3.89 (s, 3H), 3.32-3.18 (m, 4H), 2.78-2.64 (m, 2H), 2.61-2.59 (m, 7H), |
| | | | 2.33-2.29 (m, 1H), 2.17-2.16 (m, 1H), 1.81-1.75 (m, 6H), 1.55 (d, J = |
| | | | 6.80 Hz, 6H), 1.24 (s, 1H), 1.13-1.11 (m, 2H), 0.90 (s, 6H). |
| 90 | 172 | m/z = | ¹H NMR (400 MHz, DMSO-d6): δ 10.87 (s, 1H), 9.06 (bs, 1H), 8.66 |
| | | 853.54 | (d, J = 2.0 Hz, 1H), 8.26 (br s, 1H), 8.06 (s, 1H), 7.94 (d, J = 4.40 Hz, |
| | | [M + H]⁺ | 1H), 7.37 (t, J = 4.0 Hz, 1H), 7.10 (s, 1H), 7.01 (d, J = 5.2 Hz, 2H), |
| | | | 5.40 (br s, 1H), 4.45 (s, 2H), 4.43-4.30 (m, 1H), 4.23 (s, 3H), 3.65 (br s, |
| | | | 2H), 3.39 (br s, 2H), 3.25 (br s, 2H), 2.68-2.64 (m, 7H), 2.33-2.27 (m, |
| | | | 3H), 1.90-1.86 (m, 2H), 1.58-1.56 (m, 6H), 1.35 (s, 6H), 0.94 (s, 6H). |
| 91 | 173 | m/z = | ¹H NMR (400 MHz, DMSO-d6): δ 10.88 (s, 1H), 9.06 (s, 1H), 8.70 (d, |
| | | 837.54 | J = 2.80 Hz, 1H), 8.49 (d, J = 2.40 Hz, 1H), 8.11 (s, 1H), 7.96 (d, J = |
| | | [M + H]⁺ | 4.80 Hz, 1H), 7.35 (d, J = 8.00 Hz, 1H), 7.08 (s, 1H), 6.99-6.97 (m, |
| | | | 1H), 6.93-6.92 (m, 1H), 5.94 (br s, 1H), 4.59 (s, 2H), 4.32-4.31 (m, |
| | | | 1H), 4.18-4.22 (m, 5H), 3.15-3.18 (m, 4H), 2.59-2.60 (m, 9H), 2.30- |
| | | | 2.32 (m, 3H), 2.15-2.17 (m, 1H), 1.52-1.55 (m, 11H), 1.36-1.49 (m, |
| | | | 2H). |
| 92 | 174a | m/z = | ¹H NMR (400 MHz, DMSO-d6): δ 10.98 (s, 1H), 9.00 (s, 1H), 8.76 (d, |
| | | 837.58 | J = 18.00 Hz, 1H), 8.41 (d, J = 20.40 Hz, 1H), 8.27 (s, 1H), 8.06 (s, |
| | | [M + H]⁺ | 1H), 7.95 (s, 1H), 7.35 (dd, J = 2.00, 6.80 Hz, 1H), 6.98-6.96 (m, 3H), |
| | | | 6.00 (s, 1H), 4.58 (s, 3H), 4.32 (q, J = 5.20 Hz, 1H), 4.21 (s, 3H), 3.57 |
| | | | (s, 2H), 3.22 (br, 3H), 2.92 (s, 1H), 2.60-2.58 (m, 7H), 2.40 (br, 2H), |
| | | | 2.30-2.29 (m, 1H), 2.14-2.13 (m, 1H), 1.84 (d, J = 9.60 Hz, 3H), 1.74 |
| | | | (d, J = 8.40 Hz, 1H), 1.49-1.36 (m, 9H). |
| 93 | 174b | m/z = | ¹H NMR (400 MHz, DMSO-d6): δ 10.80 (s, 1H), 9.01 (s, 1H), 8.81- |
| | | 837.54 | 8.71 (m, 1H), 8.39-8.35 (m, 1H), 8.06 (s, 1H), 7.97-7.96 (m, 1H), 7.35- |
| | | [M + H]⁺ | 7.34 (m, 1H), 6.99-6.98 (m, 3H), 5.96 (s, 1H), 4.58-4.42 (m, 3H), 4.32- |
| | | | 4.31 (m, 1H), 4.21 (s, 3H), 3.53-3.52 (m, 1H), 3.22-3.20 (m, 4H), 2.92 |
| | | | (s, 1H), 2.60-2.58 (m, 8H), 2.45-2.44 (m, 2H), 2.30-2.29 (m, 1H), 2.15- |
| | | | 2.13 (m, 1H), 1.89-1.85 (m, 3H), 1.8-1.7 (m, 1H), 1.70-1.41 (m, 7H), |
| | | | 1.40-1.30 (m, 2H), |
| 94 | 175a | m/z = | ¹H NMR (400 MHz, DMSO-d6): δ 10.87 (s, 1H), 9.03 (s, 1H), 8.68 (d, |
| | | 837.54 | J = 2.00 Hz, 1H), 8.29 (s, 1H), 8.07 (s, 1H), 7.95 (d, J = 4.40 Hz, 1H), |
| | | [M + H]⁺ | 7.29 (d, J = 7.60 Hz, 1H), 7.09-6.97 (m, 3H), 5.96 (s, 1H), 4.58 (s, 2H), |
| | | | 4.47-4.42 (m, 2H), 4.32 (q, J = 5.20 Hz, 1H), 4.21 (s, 3H), 3.89 (s, 1H), |
| | | | 3.57 (t, J = 8.40 Hz, 1H), 3.47 (s, 1H), 3.05 (t, J = 8.80 Hz, 1H), 2.83 |
| | | | (t, J = 9.60 Hz, 2H), 2.73-2.62 (m, 7H), 2.46-2.41 (m, 2H), 2.31-2.25 |
| | | | (m, 1H), 2.19-2.12 (m, 1H), 1.88-1.78 (m, 4H), 1.57 (d, J = 6.80 Hz, |
| | | | 7H), 1.06 (s, 1H). |
| 95 | 175b | m/z = | ¹H NMR (400 MHz, DMSO-d6): δ 10.87 (s, 1H), 9.03 (s, 1H), 8.68 (d, |
| | | 837.50 | J = 2.40 Hz, 1H), 8.37 (s, 1H), 8.29 (d, J = 2.40 Hz, 1H), 8.07 (s, 1H), |
| | | [M + H]⁺ | 7.96 (d, J = 4.80 Hz, 1H), 7.29 (d, J = 7.60 Hz, 1H), 6.99-6.97 (m, 3H), |
| | | | 6.00 (s, 1H), 4.58 (s, 2H), 4.46 (d, J = 12.00 Hz, 2H), 4.32 (q, J = 5.20 |
| | | | Hz, 1H), 4.21 (s, 3H), 3.89 (s, 1H), 3.57 (t, J = 8.00 Hz, 1H), 3.47 (br, |
| | | | 1H), 3.05 (t, J = 8.40 Hz, 1H), 2.83 (t, J = 8.80 Hz, 2H), 2.61-2.59 (m, |
| | | | 8H), 2.39-2.31 (m, 2H), 2.18 (s, 1H), 1.82-1.78 (m, 4H), 1.57 (d, J = |
| | | | 6.80 Hz, 7H), 1.06 (s, 1H). |
| 96 | 176d | m/z = | ¹H NMR (400 MHz, DMSO-d6): δ 10.83 (s, 1H), 9.03 (s, 1H), 8.70 (d, |
| | | 839.44 | J = 2.0 Hz, 1H), 8.26-8.27 (m, 2H), 8.06 (s, 1H), 7.92 (d, J = 4.40 Hz, |
| | | [M + H]⁺ | 1H), 7.49 (d, J = 8.80 Hz, 1H), 7.12 (s, 1H), 6.91 (d, J = 8.80 Hz, 1H), |
| | | | 6.83 (s, 1H), 5.90 (s, 1H), 4.56 (s, 2H), 4.50-4.41 (m, 1H), 4.40-4.24 |
| | | | (m, 2H), 3.89 (s, 3H), 3.2 (br, 4H), 2.84-2.82 (m, 1H), 2.56-2.50 (m, |
| | | | 7H), 2.44-2.42 (m, 3H), 2.29-2.27 (m, 1H), 2.20-2.11 (m, 2H), 1.93- |
| | | | 1.90 (m, 1H), 1.56 (d, J = 6.80 Hz, 7H), 1.30-1.28 (m, 1H), 1.06-1.04 |
| | | | (m, 1H), 0.93-0.91 (m, 3H). |
| 97 | 177b | m/z = | ¹H NMR (400 MHz, DMSO-d6): δ 10.8 (s, 1H), 9.11 (m, 2H), 8.73 (d, |
| | | 839.52 | J = 2.0 Hz, 1H), 8.24 (s, 1H), 8.08 (s, 1H), 7.97 (d, J = 4.40 Hz, 1H), |
| | | [M + H]⁺ | 7.48 (d, J = 8.0 Hz, 1H), 7.21-7.06 (m, 3H), 5.96 (s, 1H), 4.57 (s, 2H), |
| | | | 4.38-4.26 (m, 5H), 4.08 (s, 1H), 3.69-3.66 (m, 2H), 3.20 (m, 9H), 2.68- |
| | | | 2.58 (m, 6H), 2.50 (m, 1H), 2.27-2.14 (m, 1H), 2.00 (brs, 1H), 1.58- |
| | | | 1.56 (m, 8H), 1.35 (s, 1H), 0.85-0.83 (d, J = 6.80 Hz, 3H). |
| 98 | 177c | m/z = | ¹H NMR (400 MHz, DMSO-d6): δ 10.87 (s, 1H), 9.04 (s, 1H), 8.70 (d, |
| | | 839.52 | J = 2.40 Hz, 1H), 8.27 (s, 1H), 8.07 (s, 1H), 7.94 (d, J = 4.40 Hz, 1H), |
| | | [M + H]⁺ | 7.38-7.37 (dd, J = 1.60, 6.80 Hz, 1H), 7.13 (s, 1H), 7.01-7.00 (m, 2H), |
| | | | 5.96 (s, 1H), 4.60 (s, 2H), 4.43 (s, 1H), 4.33-4.32 (q, J = 4.80 Hz, 2H), |
| | | | 4.24 (s, 3H), 2.84-2.81 (m, 8H), 2.67-2.64 (m, 3H), 2.61-2.59 (m, 3H), |
| | | | 2.50-2.45 (br, 1H), 2.33-2.32 (s, 1H), 2.15-2.12 (m, 3H), 1.94-1.92 (m, |
| | | | 1H), 1.70 (d, J = 6.80 Hz, 6H), 1.51 (s, 1H), 1.35 (s, 1H), 1.10 (s, 1H), |
| | | | 0.92 (d, J = 6.0 Hz, 3H). |
| 99 | 178 | m/z = | ¹H NMR (400 MHz, DMSO-d6): δ 10.86 (s, 1H), 9.02 (s, 1H), 8.70 (d, |
| | | 841.6 | J = 2.5 Hz, 1H), 8.28 (d, J = 2.5 Hz, 1H), 8.07 (s, 1H), 8.02-7.91 (m, |
| | | [M + H]⁺ | 1H), 7.42-7.36 (m, 1H), 7.10 (s, 1H), 7.04-6.96 (m, 2H), 6.02-5.90 |
| | | | (m, 1H), 4.56 (s, 2H), 4.35-4.29 (m, 1H), 4.30-4.25 (m, 1H), 4.23 (s, |
| | | | 3H), 4.15-4.04 (m, 2H), 3.39-2.72 (m, 5H), 2.71-2.57 (m, 5H), |
| | | | 2.40-2.27 (m, 3H), 2.22-2.11 (m, 1H), 1.57 (d, J = 6.9 Hz, 6H), 1.55- |
| | | | 1.47 (m, 4H). |
| 100 | 179a | m/z = | ¹H NMR (400 MHz, DMSO-d6): δ 10.88 (s, 1H), 9.25-9.21 (m, 2H), |
| | | 853.57 | 9.06 (br s, 1H), 8.64 (dd, J = 2.40, 5.60 Hz, 1H), 8.30-8.25 (m, 1H), |
| | | [M + H]⁺ | 8.16 (d, J = 2.40 Hz, 1H), 7.96-7.95 (m, 1H), 7.39-7.37 (m, 1H), 7.02- |
| | | | 7.00 (m, 1H), 6.87 (s, 1H), 5.96 (br s, 1H), 4.79 (m, 1H), 4.58 (s, 2H), |
| | | | 4.43-4.36 (m, 1H), 4.35-4.31 (m, 2H), 4.25 (d, J = 8.40 Hz, 3H), 3.36- |
| | | | 3.32 (m, 5H), 3.17-3.10 (m, 2H), 2.67 (d, J = 4.00 Hz, 6H), 2.18 (s, |
| | | | 2H), 1.91-1.89 (m, 4H), 1.58 (d, J = 6.80 Hz, 9H), 1.36-1.34 (m, 4H). |
| 101 | 179b | m/z = | ¹H NMR (400 MHz, DMSO-d6): δ 10.88 (s, 1H), 9.23 (d, J = 15.60 |
| | | 853.47 | Hz, 1H), 9.09 (s, 1H), 8.63-8.57 (m, 1H), 8.31-8.25 (m, 1H), 8.16 (d, J = |
| | | [M + H]⁺ | 2.00 Hz, 1H), 7.98-7.97 (m, 1H), 7.39-7.38 (m, 1H), 7.00-6.96 (m, |
| | | | 3H), 5.96 (s, 1H), 4.79-4.18 (m, 8H), 3.66 (br, 2H), 3.11-2.68 (m, 5H), |
| | | | 2.51-2.50 (m, 7H), 2.35-2.31 (m, 1H), 2.20-2.19 (m, 1H), 1.93-1.88 (m, |
| | | | 3H), 1.58 (d, J = 6.80 Hz, 8H), 1.20-1.19 (m, 6H). |
| 102 | 180a | m/z = | ¹H NMR (400 MHz, DMSO-d6): δ 10.81 (s, 1H), 9.03 (s, 1H), 8.78 (s, |
| | | 796.50 | 1H), 8.51 (s, 1H), 8.06 (s, 1H), 7.99 (d, J = 4.4 Hz, 1H), 7.29 (d, J = |
| | | [M + H]⁺ | 8.4 Hz, 1H), 7.12 (s, 1H), 6.50 (d, J = 8.8 Hz, 1H), 6.36 (s, 1H), 5.97 |
| | | | (bs, 1H), 5.64 (d, J = 9.2 Hz, 1H) 4.60 (s, 2H), 4.17-4.10 (m, 1H), 3.89- |
| | | | 3.73 (m, 5H), 3.67 (s, 2H), 3.05-2.95 (m, 1H), 2.70-260 (m, 3H), 2.58- |
| | | | 2.50 (m, 2H), 2.27-2.22 (m, 1H), 2.16-2.11 (m, 1H), 1.99-1.89 (m, 3H), |
| | | | 1.68-1.50 (m, 8H), 1.41-1.30 (m, 1H). 1.20-1.12 (m, 1H), 1.00-0.90 (d, J = |
| | | | 6.0 Hz, 3H). |
| 103 | 180b | m/z = | ¹H NMR (400 MHz, DMSO-d6): δ 10.81 (s, 1H), 9.45 (s, 1H), 8.77 (s, |
| | | 796.46 | 1H), 8.38 (s, 1H), 8.18 (s, 1H), 7.95 (d, J = 4.80 Hz, 1H), 7.31-7.30 (d, |
| | | [M + H]⁺ | J = 8.80 Hz, 1H), 7.20 (s, 1H), 6.69 (d, J = 7.80 Hz, 1H), 6.41 (s, 1H), |
| | | | 5.96 (brs, 1H), 5.58 (brs, 1H), 4.59 (s, 2H), 4.16-4.15 (m, 1H), 3.79- |
| | | | 3.75 (m, 3H), 3.75-3.73 (m, 2H), 3.61-3.59 (m, 3H), 2.68-2.67 (m, 3H), |
| | | | 2.58-2.51 (m, 2H), 2.58-2.51 (m, 1H), 2.11-2.07 (m, 1H), 1.69-1.66 (m, |
| | | | 12H), 1.50-1.40 (m, 1H), 0.89 (d, J = 8.80 Hz, 3H). |
| 104 | 180c | m/z = | ¹H NMR (400 MHz, DMSO-d6): δ 10.81 (s, 1H), 9.38 (s, 1H), 8.76 (s, |
| | | 796.50 | 1H), 8.46 (brs, 1H), 8.17 (s, 1H), 7.98 (d, J = 3.60 Hz, 1H), 7.30 (d, J = |
| | | [M + H]⁺ | 8.80 Hz, 1H), 7.15 (s, 1H), 6.51 (d, J = 8.80 Hz, 1H), 6.38 (s, 1H), 5.97 |
| | | | (s, 1H), 5.65 (s, 1H), 4.61 (s, 2H), 4.16-4.15 (m, 1H), 3.78-3.71 (m, |
| | | | 7H), 3.05-3.00 (m, 1H), 2.69-2.68 (m, 3H), 2.60-2.58 (m, 2H), 2.24- |
| | | | 2.22 (m, 1H), 2.13-2.11 (m, 1H), 1.94-1.90 (m, 3H), 1.70-1.34 (m, 9H), |
| | | | 1.20-1.05 (m, 1H), 0.98 (d, J = 6.00 Hz, 3H). |
| 105 | 180d | m/z = | ¹H NMR (400 MHz, DMSO-d6): δ 10.81 (s, 1H), 9.03 (s, 1H), 8.79 (s, |
| | | 796.45 | 1H), 8.42 (s, 1H), 8.06 (s, 1H), 7.96 (d, J = 4.40 Hz, 1H), 7.31 (d, J = |
| | | [M + H]⁺ | 8.80 Hz, 1H), 7.18 (s, 1H), 6.69 (d, J = 8.40 Hz, 1H), 6.41 (s, 1H), 6.00 |
| | | | (s, 1H), 5.57 (d, J = 8.40 Hz, 1H), 4.58 (s, 2H), 4.16-4.15 (m, 1H), |
| | | | 3.68-3.61 (m, 7H), 3.60 (brs, 1H), 2.68-2.67 (m, 3H), 2.51-2.50 (m, |
| | | | 2H), 2.25-2.23 (m, 1H), 2.15-2.13 (m, 1H), 1.75-1.72 (m, 4H), 1.58- |
| | | | 1.57 (m, 8H), 8 1.50-1.48 (m, 1H), 1.36 (s, 1H), 0.91 (d, J = 6.40 Hz, |
| | | | 3H). |
| 106 | 181 | m/z = 783 | ¹H NMR (400 MHz, DMSO-d6): δ = 10.87 (s, 1H), 9.02 (s, 1H), 8.77 |
| | | [M + H]⁺ | (s, 1H), 8.42 (s, 1H), 8.07 (s, 1H), 7.96-7.94 (m, 1H), 7.26-7.10 (m, |
| | | | 2H), 6.98-6.96 (m, 1H), 6.89-6.87 (m, 1H), 6.09-5.84 (m, 1H), 4.73- |
| | | | 4.48 (m, 3H), 4.32-4.30 (m, 1H), 4.18 (s, 3H), 3.74 (s, 3H), 3.18-3.13 |
| | | | (m, 1H), 2.68-2.53 (m, 6H), 2.33-2.31 (m, 1H), 2.18-2.16 (m, 1H), |
| | | | 1.94-1.92 (m, 4H), 1.81-1.52 (m, 9H) |
| 107 | 187 | m/z = | ¹H NMR (400 MHz, DMSO-d6): δ 10.82 (s, 1H), 9.02 (s, 1H), 8.69 (d, |
| | | 837.46 | J = 2.40 Hz, 1H), 8.39 (d, J = 2.40 Hz, 1H), 8.09 (s, 1H), 7.94 (d, J = |
| | | [M + H]⁺ | 4.40 Hz, 1H), 7.20 (d, J = 7.60 Hz, 1H), 7.11 (s, 1H), 7.00-6.92 (m, |
| | | | 2H), 5.96 (br s, 1H), 4.56 (s, 2H), 4.42 (d, J = 11.20 Hz, 2H), 4.28-4.29 |
| | | | (m, 1H), 4.16 (d, J = 5.20 Hz, 2H), 4.11 (s, 3H), 3.11 (d, J = 9.20 Hz, |
| | | | 2H), 2.95 (t, J = 10.00 Hz, 2H), 2.81 (t, J = 12.00 Hz, 3H), 2.66 (d, J = |
| | | | 4.80 Hz, 3H), 2.37 (d, J = 6.80 Hz, 2H), 2.12-2.14 (m, 3H), 1.67 (d, J = |
| | | | 10.80 Hz, 2H), 1.56 (d, J = 7.20 Hz, 6H), 1.36 (s, 3H), 0.98-0.95 (m, |
| | | | 2H). |
| 108 | 188a | m/z = | ¹H NMR (400 MHz, DMSO-d6): δ 10.93 (s, 1H), 9.64-9.56 (m, 1H), |
| | | 823.6 | 8.84-8.79 (m, 1H), 8.46-8.41 (m, 1H), 8.21 (s, 1H), 8.14-8.09 (m, |
| | | [M + H]⁺ | 1H), 8.04-7.98 (m, 1H), 7.49-7.41 (m, 1H), 7.14 (s, 1H), 7.11-7.05 |
| | | | (m, 2H), 6.02 (s, 1H), 4.67-4.61 (m, 2H), 4.59-4.51 (m, 2H), 4.44- |
| | | | 4.36 (m, 1H), 4.31 (s, 3H), 3.13 (s, 3H), 3.02-2.82 (m, 3H), 2.77- |
| | | | 2.66 (m, 6H), 2.60 (s, 2H), 2.52-2.30 (m, 1H), 2.27-2.18 (m, 1H), |
| | | | 2.14-1.69 (m, 3H), 1.69-1.57 (m, 7H), 1.25-0.99 (m, 6H). |
| 109 | 188b | m/z = 823 | ¹H NMR (400 MHz, DMSO-d6): δ 10.87 (s, 1H), 9.55 (s, 1H), 8.78- |
| | | [M + H]⁺ | 8.72 (m, 1H), 8.40-8.35 (m, 1H), 8.08-8.03 (m, 1H), 7.98-7.92 (m, |
| | | | 1H), 7.42-7.34 (m, 1H), 7.08 (s, 1H), 7.05-6.99 (m, 2H), 6.03-5.84 |
| | | | (m, 1H), 4.58 (s, 2H), 4.52-4.45 (m, 2H), 4.37-4.29 (m, 1H), 4.25 (s, |
| | | | 3H), 3.18-2.96 (m, 1H), 2.93-2.80 (m, 3H), 2.71-2.56 (m, 5H), |
| | | | 2.43-2.09 (m, 1H), 2.09-1.61 (m, 3H), 1.61-1.50 (m, 6H), 1.27- |
| | | | 0.90 (m, 7H). |
| 110 | 189a | m/z = | ¹H NMR (400 MHz, DMSO-d6): δ (ppm) = 10.80 (s, 1H), 9.01 (s, 1H), |
| | | 856.8 | 8.60 (d, J = 2.0 Hz, 1H), 8.24 (br d, J = 1.8 Hz, 1H), 8.03 (s, 1H), 7.88 |
| | | [M + H]⁺ | (br d, J = 4.5 Hz, 1H), 7.31 (br t, J = 4.4 Hz, 1H), 7.10-6.89 (m, 3H), |
| | | | 5.89 (br d, J = 1.6 Hz, 1H), 4.51 (s, 2H), 4.26 (br dd, J = 5.1, 9.5 Hz, |
| | | | 1H), 4.17 (s, 4H), 3.20-3.01 (m, 4H), 2.96-2.69 (m, 2H), 2.60 (d, J = |
| | | | 4.6 Hz, 4H), 2.56 (br d, J = 6.0 Hz, 3H), 2.35-2.18 (m, 2H), 2.09 (br |
| | | | dd, J = 5.4, 12.9 Hz, 1H), 2.02-1.89 (m, 1H), 1.81-1.58 (m, 2H), 1.50 |
| | | | (d, J = 6.8 Hz, 6H), 0.96 (br s, 2H). |
| 111 | 190 | m/z = | ¹H NMR (400 MHz, DMSO-d6): δ 10.84 (s, 1H), 8.42 (d, J = 2.40 Hz, |
| | | 824.50 | 1H), 8.14 (s, 1H), 7.95-7.93 (m, 3H), 7.49 (d, J = 9.20 Hz, 1H), 7.22 (s, |
| | | [M + H]⁺ | 1H), 6.90 (d, J = 8.80 Hz, 1H), 6.82 (s, 1H), 6.21 (s, 1H), 5.96 (s, 1H), |
| | | | 4.55 (s, 1H), 4.25-4.23 (m, 1H), 4.04 (d, J = 12.00 Hz, 2H), 3.88 (s, |
| | | | 3H), 3.20 (brs, 4H), 2.60-2.59 (m, 8H), 2.48 (br, 3H), 2.28-2.27 (m, |
| | | | 1H), 2.14-2.13 (m, 3H), 1.81-1.70 (m, 3H), 1.74-1.71 (m, 7H), 1.01- |
| | | | 1.10 (m, 2H). |
| 112 | 191 | m/z = | ¹H NMR (400 MHz, DMSO-d6): δ 10.87 (s, 1H), 9.26 (s, 1H), 9.03 (s, |
| | | 851.52 | 1H), 8.65 (s, 1H), 8.28 (s, 1H), 8.16 (s, 1H), 7.94 (d, J = 4.40 Hz, 1H), |
| | | [M + H]⁺ | 7.39-7.37 (m, 1H), 7.01-6.095 (m, 3H), 5.96 (br s, 1H), 4.57 (s, 2H), |
| | | | 4.32-4.25 (m, 5H), 3.65-3.45 (m, 4H), 3.27 (s, 4H), 2.68-2.50 (m, 7H), |
| | | | 2.40-2.30 (m, 1H), 2.30-2.15 (m, 1H), 2.14-1.18 (m, 3H), 1.58-1.50 (m, |
| | | | 9H), 1.36-1.24 (m, 1H), 0.9-0.8 (m, 2H). |
| 113 | 192a | m/z = | ¹H NMR (400 MHz, DMSO-d6): δ 10.89 (s, 1H), 9.12 (s, 1H), 8.93 (br |
| | | 839.59 | s, 1H), 8.73 (d, J = 2.00 Hz, 1H), 8.25 (d, J = 2.40 Hz, 1H), 8.11 (s, |
| | | [M + H]⁺ | 1H), 7.98 (d, J = 4.40 Hz, 1H), 7.49 (d, J = 7.60 Hz, 1H), 7.11-7.05 (m, |
| | | | 3H), 5.9 (br s, 1H), 4.58 (s, 2H), 4.55-4.38 (m, 2H), 4.35-4.30 (m, 1H), |
| | | | 4.26 (s, 3H), 3.78-3.71 (m, 1H), 3.60-3.50 (m, 2H), 3.20-2.80 (m, 8H), |
| | | | 2.70-2.55 (m, 5H), 2.33 (s, 1H), 2.14-2.16 (m, 2H), 1.91-1.89 (m, 1H), |
| | | | 1.57-1.58 (m, 7H), 1.39-1.13 (m, 5H). |
| 114 | 192b | m/z = | ¹H NMR (400 MHz, DMSO-d6): δ 10.89 (s, 1H), 9.12 (s, 1H), 8.93 (br |
| | | 839.52 | s, 1H), 8.73 (d, J = 2.40 Hz, 1H), 8.25 (d, J = 2.00 Hz, 1H), 8.11 (s, |
| | | [M + H]⁺ | 1H), 7.97 (d, J = 4.40 Hz, 1H), 7.49 (d, J = 7.60 Hz, 1H), 7.11-7.05 (m, |
| | | | 3H), 5.90 (s, 1H), 4.60 (s, 2H), 4.55-4.45 (m, 2H), 4.36-4.34 (m, 1H), |
| | | | 4.26 (s, 3H), 3.80-8.60 (m, 2H), 3.50-3.39 (m, 3H), 3.15-3.05 (m, 1H), |
| | | | 2.92-2.90 (m, 4H), 2.64-2.60 (m, 6H), 2.33-2.30 (m, 1H), 2.16-2.13 (m, |
| | | | 2H), 1.91-1.85 (m, 1H), 1.57 (d, J = 6.8 Hz, 7H), 1.35-1.33 (m, 4H). |
| 115 | 193a | m/z = | ¹H NMR (400 MHz, DMSO-d6): δ 10.98 (s, 1H), 9.01 (s, 1H), 8.69 (d, |
| | | 839.52 | J = 2.4 Hz, 1H), 8.29 (d, J = 2.4 Hz, 1H), 8.07 (s, 1H), 7.94 (d, J = 4.8 |
| | | [M + H]⁺ | Hz, 1H), 7.38 (dd, J = 4.4, 2.0 Hz, 1H), 7.10 (s, 1H), 7.02-6.99 (m, |
| | | | 2H), 5.96 (s, 1H), 4.57 (s, 2H), 4.50-4.48 (m, 2H) 4.35-4.31 (m, 1H), |
| | | | 4.24 (s, 3H), 3.31 (br s, 2H), 3.10 (bs, 6H), 2.79-2.67 (m, 7H), 2.64- |
| | | | 2.60 (m, 1H), 2.50-2.49 (m, 1H), 2.30-2.28 (m, 1H), 1.58-1.56 (m, 9H) |
| | | | 1.06-1.03, (m, 2H), 0.97-0.95 (m, 3H). |
| 116 | 194b | m/z = | ¹H NMR (400 MHz, DMSO-d6): δ 10.87 (s, 1H), 9.12 (s 1H), 8.69 (d, |
| | | 854.49 | J = 2.4 Hz, 1H), 8.29 (d, J = 2.4 Hz, 1H), 8.09 (s, 1H), 7.94 (d, J = 4.4 |
| | | [M + H]⁺ | Hz, 1H), 7.40-7.28 (m, 2H), 7.10-6.95 (m, 3H), 5.96 (bs, 1H), 4.57-4.52 |
| | | | (m, 4H), 4.34-4.31 (m, 1H), 4.25 (s, 3H), 3.327 (bs, 2H), 2.78-2.58 (m, |
| | | | 9H), 2.31 (bs, 1H), 2.19-2.14 (m, 1H), 1.92-1.89 (m, 1H), 1.72-1.56 (m, |
| | | | 10H), 1.28-1.17 (m, 4H), 0.97 (s, 3H), 0.90-0.80 (m, 1H). |
| 117 | 195a | m/z = | ¹H NMR (400 MHz, DMSO-d6): δ 10.88 (s, 1H), 9.24 (s, 1H), 9.09 (br |
| | | 837.46 | s, 1H), 8.68 (d, J = 2.4 Hz, 1H), 8.30 (d, J = 2.0 Hz, 1H), 8.18 (s, 1H), |
| | | [M + H]⁺ | 7.95 (d, J = 6.8 Hz, 1H), 7.15 (s, 1H), 7.04-7.01 (m, 2H), 5.97 (s, 1H), |
| | | | 4.58 (s, 4H), 4.35-4.31 (m, 1H) 4.25 (s, 3H), 3.60-3.56 (m, 2H), 3.39- |
| | | | 3.28 (s, 7H), 2.75-2.66 (m, 7H), 2.50-2.49 (m, 1H), 2.30-2.17 (m, 2H), |
| | | | 1.81-1.77 (m, 2H) 1.58-1.56 (m, 8H), 1.35-1.30 (m, 3H). |
| 118 | 197b | m/z = | ¹H NMR (400 MHz, DMSO-d6): δ 10.88 (s, 1H), 9.04 (s, 1H), 8.69 (d, |
| | | 851.45 | J = 2.00 Hz, 1H), 8.30-8.28 (m, 1H), 8.07 (s, 1H), 7.96 (d, J = 4.40 Hz, |
| | | [M + H]⁺ | 1H), 7.39 (d, J = 6.80 Hz, 1H), 7.10 (s, 1H), 7.09-7.00 (m, 2H), 5.97 |
| | | | (br s, 1H), 4.58 (s, 2H), 4.36 (br s, 2H), 4.40-4.20 (m, 4H), 3.25-3.00 |
| | | | (m, 5H), 2.84-2.81 (m, 3H), 2.73-2.62 (m, 5H), 2.17-2.09 (m, 4H), |
| | | | 1.79-1.70 (m, 3H), 1.58 (d, J = 6.80 Hz, 6H), 1.20-0.90 (m, 8H). |
| 119 | 199 | m/z = | ¹H NMR (400 MHz, DMSO-d6): δ (ppm) = 10.81 (s, 1H), 9.02 (s, 1H), |
| | | 809.8 | 8.69 (d, J = 2.4 Hz, 1H), 8.30 (d, J = 2.5 Hz, 1H), 8.07 (s, 1H), 7.96 (br |
| | | [M + H]⁺ | d, J = 4.8 Hz, 1H), 7.31 (d, J = 8.8 Hz, 1H), 7.09 (s, 1H), 6.51 (dd, J = |
| | | | 1.1, 8.9 Hz, 1H), 6.37 (s, 1H), 5.96 (br s, 1H), 5.68 (br d, J = 8.0 Hz, |
| | | | 1H), 4.59 (s, 2H), 4.17 (dd, J = 5.2, 8.7 Hz, 1H), 3.80 (s, 3H), 3.63 (br |
| | | | s, 4H), 2.69 (d, J = 4.8 Hz, 3H), 2.60 (br t, J = 6.6 Hz, 2H), 2.31-2.19 |
| | | | (m, 1H), 2.19-2.09 (m, 1H), 1.90-1.79 (m, 2H), 1.74 (br d, J = 11.6 |
| | | | Hz, 2H), 1.57 (d, J = 6.9 Hz, 6H), 1.51 (br d, J = 2.4 Hz, 2H), 1.44- |
| | | | 1.25 (m, 6H). |
| 120 | 200 | m/z = | ¹H NMR (400 MHz, DMSO-d6): 10.90 (s, 1H), 9.11 (s, 1H), 8.71 (s, |
| | | 851.41 | 1H), 8.60 (br s, 1H), 8.25 (s 1H), 8.10 (s, 1H), 7.96 (d, J = 4.4 Hz, 1H), |
| | | [M + H]⁺ | 7.50 (d, J = 7.6 Hz 1H), 7.11-7.06 (m, 3H), 5.95 (br s, 1H), 4.57 (s, |
| | | | 2H), 4.47(br s, 2H), 4.38-4.34 (m, 1H), 4.26 (s, 3H), 3.62 (br s, 2H), |
| | | | 3.37-3.30 (m, 3H), 3.31-3.29 (m, 1H), 2.94-2.85 (m, 4H), 2.67-2.61 (m, |
| | | | 4H), 2.39 (s, 1H), 2.18-1.98 (m, 3H), 1.71 (d, J = 10 Hz 1H), 1.58 (d, J = |
| | | | 6.8 Hz, 6H), 1.40 (s, 3H), 1.35 (m, 3H), 1.30-1.10 (m, 3H). |
| 121 | 201a | m/z = | ¹H NMR (400 MHz, DMSO-d6): δ 10.84 (s, 1H), 9.02 (s 1H), 8.70 (d, |
| | | 852.44 | J = 2.4 Hz, 1H), 8.30 (d, J = 2.0 Hz, 1H), 8.07 (s, 1H), 7.94 (d, J = 4.4 |
| | | [M + H]⁺ | Hz, 1H), 7.46 (d, J = 8.8 Hz, 1H), 7.10 (s, 1H), 6.90 (d, J = 8.8 Hz, |
| | | | 1H), 6.80 (s, 1H), 5.95 (bs, 1H), 4.57-4.54 (m, 4H), 4.26-4.22 (m, 1H), |
| | | | 3.88-3.84 (m, 6H), 3.79-3.74 (m, 2H), 2.66-2.59 (m, 7H), 2.30-2.27 (m, |
| | | | 1H), 2.17-2.13 (m, 1H), 1.81-1.55 (m, 14H), 1.30-1.17 (m, 2H), 0.97 (s, |
| | | | 3H). |
| 122 | 202 | m/z = | ¹H NMR (400 MHz, DMSO-d6): δ = 10.81 (s, 1H), 9.03 (s, 1H), 8.80 |
| | | 754.5 | (s, 1H), 8.41 (d, J = 2.0 Hz, 1H), 8.07 (s, 1H), 7.96 (d, J = 4.8 Hz, 1H), |
| | | [M + H]⁺ | 7.33 (d, J = 8.8 Hz, 1H), 7.17 (s, 1H), 6.48 (d, J = 8.8 Hz, 1H), 6.26 (s, |
| | | | 1H), 6.18 (d, J = 6.1 Hz, 1H), 5.98 (d, J = 5.6 Hz, 1H), 4.60 (s, 2H), |
| | | | 4.18 (d, J = 5.2, 8.4 Hz, 1H), 4.10 (s, 2H), 3.96 (s, 2H), 3.91-3.84 (m, |
| | | | 1H), 3.80 (s, 3H), 2.76-2.65 (m, 5H), 2.59 (d, J = 6.1 Hz, 2H), 2.25 (t, |
| | | | J = 8.3, 14.0 Hz, 1H), 2.18-2.03 (m, 3H), 1.57 (d, J = 6.8 Hz, 6H) |
| 123 | 203 | m/z = | ¹H NMR (400 MHz, DMSO-d6): δ (ppm) = 10.88 (s, 1H), 9.11 (s, 1H), |
| | | 840.3 | 8.70 (s, 1H), 8.27 (br d, J = 1.0 Hz, 1H), 8.15-8.07 (m, 1H), 7.98 (br s, |
| | | [M + H]⁺ | 1H), 7.59 (d, J = 8.1 Hz, 1H), 7.45-7.19 (m, 1H), 7.17-7.05 (m, 2H), |
| | | | 6.07-5.84 (m, 1H), 5.39 (br s, 1H), 4.59 (s, 2H), 4.36 (dd, J = 5.1, 10.0 |
| | | | Hz, 1H), 4.30-4.05 (m, 5H), 3.79-3.64 (m, 3H), 3.40-3.28 (m, 4H), |
| | | | 3.18 (br d, J = 2.6 Hz, 2H), 2.75-2.57 (m, 5H), 2.41-2.22 (m, 3H), |
| | | | 2.22-2.10 (m, 2H), 2.09-1.93 (m, 2H), 1.81-1.68 (m, 2H), 1.67- |
| | | | 1.50 (m, 8H) |
| 124 | 206 | m/z = | ¹H NMR (400 MHz, DMSO-d6): δ = 10.90-10.75 (m, 1H), 9.11-8.60 |
| | | 319.1 | (m, 2H), 8.50-8.19 (m, 1H), 8.06-7.85 (m, 2H), 7.47(d, J = 8.8 Hz, |
| | | [M + H]⁺ | 1H), 7.25-7.10 (m, 1H), 7.07-6.76 (m, 1H), 6.45-6.17 (m, 2H), 6.12- |
| | | | 5.79 (m, 1H), 4.56 (s, 2H), 4.23 (dd, J = 5.2, 8.8 Hz, 1H), 3.84 (s, |
| | | | 3H), 3.62 (s, 2H), 3.58-3.50 (m, 2H), 2.69 (d, J = 4.8 Hz, 3H), 2.62- |
| | | | 2.58 (m, 2H), 2.32-2.22 m, 2H), 2.21-2.03 (m, 2H), 1.97-1.89 (m, |
| | | | 2H), 1.88-1.78 (m, 2H), 1.59 (d, J = 6.8 Hz, 6H), 1.55-1.44 (m, 2H), |
| | | | 1.39-1.21 (m, 2H), 1.08 (t, J = 6.8 Hz, 1H), 0.94 (t, J = 7.2 Hz, 1H) |
| 125 | 207 | m/z = | ¹H NMR (400 MHz, DMSO-d6): δ = 10.83 (s, 1H), 9.03 (s, 1H), 8.79 |
| | | 768.6 | (d, J = 2.0 Hz, 1H), 8.42 (d, J = 2.4 Hz, 1H), 8.07 (s, 1H), 7.96 (d, J = |
| | | [M + H]⁺ | 4.8 Hz, 1H), 7.47 (d, J = 8.8 Hz, 1H), 7.16 (s, 1H), 6.80 (d, J = 1.2, 9.2 |
| | | | Hz, 1H), 6.63 (s, 1H), 6.08-5.86 (m, 1H), 4.60 (s, 2H), 4.24 (d, J = |
| | | | 5.2, 9.2 Hz, 1H), 4.12 (s, 2H), 4.04 (t, J = 8.0 Hz, 1H), 3.94 (s, 2H), |
| | | | 3.87 (s, 3H), 2.84 (s, 3H), 2.68 (d, J = 4.6 Hz, 3H), 2.65-2.56 (m, 4H), |
| | | | 2.34-2.22 (m, 3H), 2.21-2.10 (m, 1H), 1.57 (d, J = 6.8 Hz, 6H) |
| 126 | 208a | m/z = | ¹H NMR (400 MHz, DMSO-d6): δ 10.87 (s, 1H), 9.25 (d, J = 8.00 Hz, |
| | | 883.46 | 1H), 8.71 (d, J = 2.00 Hz, 1H), 8.22 (s, 2H), 7.94 (d, J = 4.80 Hz, 1H), |
| | | [M + H]⁺ | 7.39-7.38 (m, 1H), 7.01-6.91 (m, 3H), 6.11-5.70-5.50 (m, 1H), 4.80- |
| | | | 4.00 (m, 7H), 3.90-3.80 (m, 1H), 3.60-3.00 (m, 9H), 2.80-2.58 (m, 7H), |
| | | | 2.40-2.28 (m, 2H), 1.99-1.94 (m, 4H), 1.57-1.47 (m, 8H), 1.30-1.0 9 |
| | | | (m, 6H). |
| 127 | 208b | m/z = | ¹H NMR (400 MHz, DMSO-d6): δ 10.80 (s, 1H), 9.01 (s, 1H), 8.73 (s, |
| | | 883.48 | 1H), 8.39 (s, 1H), 8.17 (s, 1H), 8.07 (s, 1H), 7.92 (d, J = 4.4 Hz, 1H), |
| | | [M + H]⁺ | 7.36-7.34 (m, 1H), 7.18 (s, 1H), 7.029-6.99 (m. 2H), 5.96 (br s, 1H), |
| | | | 4.56 (s, 2H), 4.34-4.31 (m, 1H), 4.22-4.13 (m, 5H), 3.80 (br s, 1H), |
| | | | 3.46-3.45 (m, 4H), 3.14-3.17 (m, 2H), 2.70-2.58 (m, 6H), 2.50-2.40 (m, |
| | | | 1H), 2.40-2.30 (m, 1H), 2.32-2.20 (m, 1H), 2.05-1.90 (m, 2H), 1.90- |
| | | | 1.85 (m, 2H), 1.56 (d, J = 6.80 Hz, 6H), 1.50-1.35 (br s, 3H), 1.20-1.08 |
| | | | (m, 6H). |
| 128 | 209 | m/z = | ¹H NMR (400 MHz, DMSO-d6): δ 10.96 (br s, 1H), 8.68 (d, J = 2.00 |
| | | 837.46 | Hz, 1H), 8.30 (d, J = 2.40 Hz, 1H), 8.06 (s, 1H), 7.95 (d, J = 4.80 Hz, |
| | | [M + H]⁺ | 1H), 7.51-7.46 (m, 2H), 7.12-7.08 (m, 2H), 6.01 (s, 1H), 4.58 (s, 2H), |
| | | | 4.47 (d, J = 11.20 Hz, 2H), 4.36-4.32 (m, 1H), 4.18 (s, 3H), 3.60-3.58 |
| | | | (m, 4H), 3.10-3.07 (m, 2H), 2.85-2.67 (m, 2H), 2.66-2.54 (m, 7H), |
| | | | 2.36-2.35 (m, 1H), 2.18-2.15 (m, 2H), 2.05 (d, J = 8.00 Hz, 1H), 1.82- |
| | | | 1.79 (m, 2H), 1.57 (d, J = 6.80 Hz, 6H), 1.36 (s, 3H), 1.13-1.10 (m, |
| | | | 1H). |
| 129 | 211 | m/z = | ¹H NMR (400 MHz, DMSO-d6): δ 10.85 (s, 1H), 9.18 (s, 1H), 8.68 (d, |
| | | 839.44 | J = 2.40 Hz, 1H), 8.28 (d, J = 2.00 Hz, 1H), 8.11 (s, 1H), 7.93 (d, J = |
| | | [M + H]⁺ | 4.40 Hz, 1H), 7.53 (d, J = 9.20 Hz, 1H), 7.10 (s, 1H), 6.95 (d, J = 9.20 |
| | | | Hz, 1H), 6.90 (s, 1H), 400 MHz, DMSO-d6: δ 5.95 (s, 1H), 4.56 (s, |
| | | | 2H), 4.44 (d, J = −12.00 Hz, 2H), 4.29-4.25 (m, 1H), 3.82-3.75 (m, 2H), |
| | | | 3.64-3.62 (m, 2H), 3.25-3.18 (m, 4H), 3.01-2.95 (m, 3H), 2.67-2.64 (m, |
| | | | 5H), 2.51-2.50 (m, 1H), 2.30-2.29 (m, 1H), 1.65-1.61 (m, 2H), 1.58 (d, |
| | | | J = −8.00 Hz, 8H). |
| 131 | 213 | m/z = | ¹H NMR (400 MHz, DMSO-d6): δ = 10.86 (s, 1H), 9.05 (s, 1H), 8.80 |
| | | 755.5 | (s, 1H), 8.41 (d, J = 2.4 Hz, 1H), 8.07 (s, 1H), 7.97 (d, J = 4.4 Hz, 1H), |
| | | [M + H]⁺ | 7.56 (d, J = 8.8 Hz, 1H), 7.19-7.10 (m, 1H), 6.90 (d, J = 1.6 Hz, 1H), |
| | | | 6.73-6.62 (m, 1H), 6.04-5.88 (m, 1H), 4.80 (t, J = 6.8 Hz, 1H), 4.59 |
| | | | (s, 2H), 4.29 (dd, J = 5.2, 9.6 Hz, 1H), 4.11 (s, 2H), 4.00 (s, 2H), 3.91 |
| | | | (s, 3H), 2.91-2.81 (m, 2H), 2.68 (d, J = 4.4 Hz, 3H), 2.64-2.57 (m, |
| | | | 2H), 2.34-2.26 (m, 3H), 2.20-2.09 (m, 1H), 1.57 (d, J = 6.8 Hz, 6H) |
| 132 | 216a | m/z = | ¹H NMR (400 MHz, DMSO-d6): δ 10.82 (s, 1H), 9.08 (s, 1H), 8.67 (d, |
| | | 825.4 | J = 2.5 Hz, 1H), 8.29 (d, J = 2.6 Hz, 1H), 8.08 (s, 1H), 7.92 (d, J = 5.0 |
| | | [M + H]⁺ | Hz, 1H), 7.45 (d, J = 8.8 Hz, 1H), 7.12 (s, 1H), 6.58-6.50 (m, 1H), |
| | | | 6.34 (d, J = 1.9 Hz, 1H), 5.95 (s, 1H), 4.56 (s, 2H), 4.22 (dd, J = 8.8, 5.2 |
| | | | Hz, 1H), 3.99 (d, J = 13.1 Hz, 2H), 3.83 (s, 3H), 3.46 (t, J = 8.3 Hz, |
| | | | 1H), 3.37 (d, J = 4.9 Hz, 1H), 3.29-3.22 (m, 1H), 3.05-2.87 (m, 3H), |
| | | | 2.66 (d, J = 4.7 Hz, 3H), 2.64-2.56 (m, 2H), 2.40 (s, 1H), 2.32-2.08 |
| | | | (m, 3H), 1.80 (dd, J = 12.5, 7.2 Hz, 1H), 1.56 (d, J = 6.9 Hz, 6H), 0.99 |
| | | | (d, J = 6.2 Hz, 3H). |
| 133 | 216c | m/z = | ¹H NMR (400 MHz, DMSO-d6): δ 10.75 (s, 1H), 9.01 (s, 1H), 8.60 (d, |
| | | 825.4 | J = 2.5 Hz, 1H), 8.22 (d, J = 2.6 Hz, 1H), 8.01 (s, 1H), 7.84 (d, J = 5.0 |
| | | [M + H]⁺ | Hz, 1H), 7.38 (d, J = 8.8 Hz, 1H), 7.05 (s, 1H), 6.52-6.43 (m, 1H), |
| | | | 6.26 (s, 1H), 5.88 (s, 1H), 4.49 (s, 2H), 4.15 (dd, J = 8.8, 5.1 Hz, 1H), |
| | | | 3.98 (s, 2H), 3.77 (s, 3H), 3.36 (dd, J = 19.2, 11.1 Hz, 2H), 3.19 (d, J = |
| | | | 26.0 Hz, 2H), 3.08-2.99 (m, 1H), 2.87 (dd, J = 29.5, 12.4 Hz, 2H), |
| | | | 2.75-2.49 (m, 6H), 2.35-1.97 (m, 6H), 1.69-1.60 (m, 1H), 1.49 (d, |
| | | | J = 6.9 Hz, 7H), 0.90 (d, J = 6.1 Hz, 3H). |
| 134 | 216d | m/z = | ¹H NMR (400 MHz, DMSO-d6): δ 10.82 (s, 1H), 9.08 (s, 1H), 8.67 (d, |
| | | 825.4 | J = 2.5 Hz, 1H), 8.29 (d, J = 2.5 Hz, 1H), 8.08 (s, 1H), 7.92 (d, J = 4.9 |
| | | [M + H]⁺ | Hz, 1H), 7.45 (d, J = 8.9 Hz, 1H), 7.12 (s, 1H), 6.53 (dd, J = 10.3, 8.4 |
| | | | Hz, 1H), 6.34 (d, J = 1.8 Hz, 1H), 5.95 (s, 1H), 4.22 (dd, J = 8.7, 5.1 |
| | | | Hz, 1H), 4.00 (s, 2H), 3.83 (s, 3H), 3.46 (t, J = 8.4 Hz, 1H), 3.37 (s, |
| | | | 2H), 3.27 (d, J = 14.3 Hz, 2H), 3.10-2.84 (m, 3H), 2.66 (d, J = 4.7 Hz, |
| | | | 3H), 2.60 (d, J = 6.2 Hz, 2H), 2.41 (s, 4H), 2.31-2.08 (m, 4H), 1.80 |
| | | | (dd, J = 12.4, 7.3 Hz, 1H), 1.56 (d, J = 6.9 Hz, 7H), 0.99 (d, J = 6.1 Hz, |
| | | | 3H). |
| 135 | 217a | m/z = | ¹H NMR (400 MHz, DMSO-d6): δ 10.90 (s, 1H), 8.45-8.38 (m 2H), |
| | | 838.45 | 8.00-7.99 (m, 2H), 7.95 (d, J = 4.40 Hz, 1H), 7.58-7.55 (m, 1H), 7.08- |
| | | [M + H]⁺ | 6.38 (m, 4H), 6.23 (s, 1H), 5.97 (br s, 1H), 4.56 (s, 2H), 4.36-4.35 (m, |
| | | | 1H), 4.27 (s, 3H), 4.05-4.03 (m, 2H), 3.67-3.64 (m, 2H), 3.28-3.25 (m, |
| | | | 2H), 3.11-3.01 (m, 5H), 2.84-2.81 (m, 2H), 2.78-2.68 (m, 3H), 2.63- |
| | | | 2.61 (m, 2H), 2.36 (s, 1H), 2.22-2.00 (m, 2H), 1.78-1.75 (m, 2H), 1.58 |
| | | | (d, J = 6.8 Hz, 6H), 1.23-1.18 (m, 1H), 0.86 (d, J = 6.0 Hz, 3H). |
| 136 | 217b | m/z = | ¹H NMR (400 MHz, DMSO-d6): δ 10.90 (s, 1H), 9.42 (s, 1H), 8.08- |
| | | 838.45 | 8.04 (m, 3H), 7.57 (d, J = 2.80 Hz, 1H), 7.15-7.13 (m, 3H), 6.29 (s, |
| | | [M + H]⁺ | 1H), 5.95 (brs, 1H), 4.36-4.35 (m, 1H), 4.27 (s, 3H), 4.07 (d, J = 10.80 |
| | | | Hz, 2H), 3.91 (brs, 2H), 3.60-3.55 (m, 3H), 3.25-3.20 (m, 2H), 3.14 (s, |
| | | | 7H), 2.82-2.79 (m, 2H), 2.63-2.61 (m, 2H), 2.35 (s, 1H), 2.09-2.07 (m, |
| | | | 3H), 1.85-1.79 (m, 2H), 1.58 (d, J = 6.80 Hz, 6H), 1.36 (s, 2H), 0.86 (d, |
| | | | J = 6.00 Hz, 3H). |
| 137 | 218a | m/z = | ¹H NMR (400 MHz, DMSO-d6): δ 10.85 (s, 1H), 8.96 (s, 1H), 8.89- |
| | | 811.43 | 8.81 (m, 1H), 8.45-8.32 (m, 1H), 8.14 (s, 1H), 8.07 (s, 1H), 7.93 (s, |
| | | [M + H]⁺ | 1H), 7.49 (d, J = 9.20 Hz, 1H), 7.14 (s, 1H), 6.91 (d, J = 8 Hz, 1H), |
| | | | 6.83 (br s, 1H), 4.56 (s, 2H), 4.28-4.24 (m, 1H), 3.89 (s, 3H), 3.65-3.55 |
| | | | (m, 2H), 3.44-3.39 (m, 1H), 3.21 (m, 5H), 2.67-2.50 (m, 10H), 2.50- |
| | | | 2.41 (m, 2H), 2.41-2.39 (m, 1H), 2.31-2.30 (m, 1H), 2.05 (m, 1H), |
| | | | 1.91-1.47 (m, 7H). |
| 138 | 218b | m/z = | ¹H NMR (400 MHz, DMSO-d6): δ 10.90 (s, 1H), 8.97 (s, 1H), 8.82- |
| | | 811.39 | 8.89 (m, 1H), 8.30-8.45 (m, 1H), 8.07 (s, 1H), 7.92 (s, 1H), 7.49 (d, J = |
| | | [M + H]⁺ | 9.20 Hz, 1H), 7.14 (s, 1H), 6.83-6.90 (m, 2H), 5.93 (s, 1H), 4.96 (s, |
| | | | 2H), 4.24-4.25 (m, 1H), 3.89 (s, 3H), 3.39-3.42 (m, 3H), 3.32 (brs, 5H), |
| | | | 2.58-2.59 (m, 7H), 2.29-2.39 (m, 5H), 2.17-2.18 (m, 1H), 2.07-2.14 (m, |
| | | | 1H), 1.40-1.70 (m, 8H). |
| 139 | 219 | m/z = | ¹H NMR (400 MHz, DMSO-d6): 10.84 (s, 1H), 9.11 (s, 1H), 8.66 (d, J = |
| | | 840.43 | 2.0 Hz, 1H), 8.30 (d, J = 2.0 Hz, 1H), 8.12 (s 1H), 7.92 (d, J = 4.4 |
| | | [M + H]⁺ | Hz, 1H), 7.52 (d, J = 8.8 Hz 1H), 7.15 (s, 1H), 6.94-6.88 (m, 2H), 5.96 |
| | | | (bs, 1H), 4.57 (s, 2H), 4.35-4.24 (m, 1H), 3.89 (s, 3H), 3.65 (br s, 4H), |
| | | | 3.40-3.22 (m, 11H), 2.75-2.59 (m, 5H), 2.40.-2.30 (m, 1H), 2.20-2.10 |
| | | | (m, 1H), 1.56 (d, J = 6.8 Hz, 6H). |
| 140 | 220 | m/z = | ¹H NMR (400 MHz, DMSO-d6): δ 10.88 (s, 1H), 9.11 (s, 1H), 8.18 (d, |
| | | 839.36 | J = 3.20 Hz, 1H), 8.30 (d, J = 2.00 Hz, 1H), 8.12 (s, 1H), 7.94 (d, J = |
| | | [M + H]⁺ | 4.80 Hz, 1H), 7.61 (d, J = 8.40 Hz, 1H), 7.44 (s, 1H), 7.16 (s, 1H), 7.04 |
| | | | (d, J = 8.40 Hz, 1H), 5.97 (br s, 1H), 4.58 (s, 2H), 4.34-4.32 (m, 1H), |
| | | | 3.97 (s, 3H), 3.80-3.77 (m, 2H), 3.65 (br s, 4H), 3.32 (s, 4H), 2.90-2.87 |
| | | | (m, 3H), 2.72-2.60 (m, 5H), 2.40-2.30 (s, 1H), 2.20-2.14 (m, 1H), 1.83- |
| | | | 1.80 (m, 2H), 1.74-1.71 (m, 2H), 1.69-1.58 (m, 6H). |
| 141 | 221b | m/z = | ¹H NMR (400 MHz, DMSO-d6): δ = 10.83 (s, 1H), 9.06 (s, 1H), 8.79 |
| | | 850.2 | (d, J = 1.9 Hz, 1H), 8.50-8.43 (m, 1H), 8.09 (s, 1H), 8.01 (br d, J = |
| | | [M + H]⁺ | 1.1 Hz, 1H), 7.38-7.27 (m, 1H), 7.15 (s, 1H), 6.51-6.41 (m, 2H), 6.06- |
| | | | 5.90 (m, 1H), 5.75 (br d, J = 9.8 Hz, 1H), 4.63 (br s, 2H), 4.18 (dd, J = |
| | | | 5.2, 8.7 Hz, 1H), 3.93-3.84 (m, 1H), 3.82 (s, 3H), 3.79-3.62 (m, |
| | | | 4H), 2.68 (d, J = 4.6 Hz, 3H), 2.64-2.52 (m, 3H), 2.49-2.41 (m, 1H), |
| | | | 2.32-2.20 (m, 2H), 2.19-2.10 (m, 1H), 2.03 (br d, J = 10.4 Hz, 1H), |
| | | | 1.93 (br d, J = 12.0 Hz, 1H), 1.79-1.63 (m, 2H), 1.58 (d, J = 7.0 Hz, |
| | | | 6H), 1.37-1.21 (m, 1H) |
| 142 | 222 | m/z = | ¹H NMR (400 MHz, DMSO-d6): δ = 10.88 (s, 1H), 9.01 (s, 1H), 8.78 |
| | | 800.7 | (d, J = 1.6 Hz, 1H), 8.52-8.38 (m, 1H), 8.06 (s, 1H), 7.96 (d, J = 3.6 |
| | | [M + H]⁺ | Hz, 1H), 7.31 (dd, J = 4.4, 8.8 Hz, 1H), 7.13 (s, 1H), 6.95 (dd, J = 9.2, |
| | | | 10.4 Hz, 1H), 6.10-5.81 (m, 1H), 4.59 (s, 1H), 4.32 (dd, J = 5.2, 9.6 |
| | | | Hz, 1H), 4.20 (s, 4H), 3.74 (s, 2H), 3.62 (s, 2H), 2.99-2.84 (m, 1H), |
| | | | 2.71-2.59 (m, 5H), 2.35-2.28 (m, 1H), 2.16 (dd, J = 5.6, 13.2 Hz, |
| | | | 1H), 1.90 (d, J = 11.2 Hz, 2H), 1.84-1.77 (m, 2H), 1.62-1.31 (m, |
| | | | 11H) |
| 143 | 223 | m/z = | ¹H NMR (400 MHz, DMSO-d6): δ 10.90 (s, 1H), 9.12 (s, 1H), 8.66 (d, |
| | | 839.44 | J = 2.4 Hz, 1H), 8.31 (d, J = 2.4 Hz, 1H), 8.12 (s, 1H), 7.94 (d, J = 4.4 |
| | | [M + H]⁺ | Hz, 1H), 7.41 (d, J = 7.2 Hz, 1H), 7.16 (s, 1H), 7.06-7.00 (m, 2H), 5.97 |
| | | | (bs, 1H), 4.58 (s, 2H), 4.36-4.32 (m, 1H), 4.26 (s, 3H), 3.72-3.63 (m, |
| | | | 6H), 3.30-3.15 (m, 8H), 2.67-2.66 (m, 2H), 2.63-2.61 (m, 5H), 2.40- |
| | | | 2.38 (m, 1H), 2.18-2.14 (m, 1H), 1.57 (d, J = 6.8 Hz, 6H). |
| 144 | 225 | m/z = | ¹H NMR (400 MHz, DMSO-d6): δ 10.87 (s, 1H), 9.06 (s, 1H), 8.66 (d, |
| | | 826.33 | J = 2.4 Hz, 1H), 8.31 (d, J = 2.4 Hz, 1H), 8.08 (s, 1H), 7.94 (d, J = 4.4 |
| | | [M + H]⁺ | Hz, 1H), 7.36 (d, J = 4.8 Hz, 1H), 7.08 (s, 1H), 7.01-7.00 (m, 2H), 5.96 |
| | | | (br s, 1H), 4.581(s, 2H), 4.34-4.31 (m, 1H), 4.24(s, 3H), 4.05 (br, 2H), |
| | | | 3.75-3.60 (m, 2H), 3.265 (br s, 3H), 2.984 (br, 1H), 2.74-2.61 (m, 8H), |
| | | | 2.29 (s, 1H), 2.26-2.13 (m, 1H), 2.10-2.05 (m, 1H), 1.90-1.86 (m, 3H), |
| | | | 1.70-1.52 (m, 7H), 1.65 (br, 2H). |
| 145 | 227a | m/z = | ¹H NMR (400 MHz, DMSO-d6): δ 10.84 (s, 1H), 9.80 (br s, 1H), 9.25 |
| | | 881.46 | (s, 1H), 8.66 (d, J = 2.40 Hz, 1H), 8.26 (d, J = 2.40 Hz, 1H), 8.18 (s, |
| | | [M + H]⁺ | 1H), 7.95 (d, J = 4.40 Hz, 1H), 7.49 (d, J = 8.80 Hz, 1H), 7.21-6.96 (m, |
| | | | 6H TFA salt), 6.91 (d, J = 9.20 Hz, 1H), 6.86 (s, 1H), 5.99 (br s, 1H), |
| | | | 4.58-4.50 (m, 4H), 4.26-4.23 (m, 1H), 3.94-3.88 (m, 3H), 3.32-3.30 (m, |
| | | | 3H), 3.18-3.12 (m, 3H), 3.00-2.88 (m, 5H), 2.68-2.61 (m, 8H), 2.41- |
| | | | 2.35 (m, 2H), 2.31-2.27 (m, 3H), 1.94-1.91 (m, 2H), 1.56 (d, J = 6.80 |
| | | | Hz, 8H), 1.35 (s, 2H). |
| 146 | 228a | m/z = | ¹H NMR (400 MHz, DMSO-d6): δ 10.83 (s, 1H), 9.08 (s, 1H), 8.67- |
| | | 811.39 | 8.66 (d, J4 Hz, 1H), 8.31-8.30 (d, J = 4.0 Hz, 1H), 8.14 (s, 1H), 7.93- |
| | | [M + H]⁺ | 7.92 (d, J = 4.0 Hz, 1H), 7.46-7.44 (d, J = 8.0 Hz, 1H), 7.10 (s, 1H), |
| | | | 6.55-6.53 (d, J = 8.0 Hz, 1H), 6.34 (s, 1H), 5.98 (br, 1H), 4.57 (s, 2H), |
| | | | 4.25-4.24 (m, 1H), 3.84 (s, 3H), 3.68-3.59 (m, 4H), 3.50-3.47 (m, 1H), |
| | | | 3.40 (br, 2H), 3.10-3.01 (m, 1H), 2.67-2.62 (m, 6H), 2.49-2.43 (m, 5H), |
| | | | 2.33-2.18 (m, 1H), 2.20-2.09 (m, 2H), 1.77-1.74 (m, 2H), 1.56-1.55 (d, |
| | | | J = 4.0 Hz, 6H). |
| 147 | 229 | m/z = | ¹H NMR (400 MHz, DMSO-d6): δ (ppm) = 10.84 (s, 1H), 9.03 (s, 1H), 8.68 |
| | | 426.3 | (d, J = 2.0 Hz, 1H), 8.28 (br s, 1H), 8.06 (s, 1H), 7.95 (br d, J = 4.6 Hz, 1H), |
| | | [M/2 + H]⁺ | 7.50 (br d, J = 8.5 Hz, 1H), 7.08 (s, 1H), 6.92 (br d, J = 8.9 Hz, 1H), |
| | | | 6.83 (br s, 1H), 5.95 (br d, J = 1.0 Hz, 1H), 4.58 (s, 2H), 4.26 (br dd, J = |
| | | | 5.1, 8.9 Hz, 1H), 3.89 (s, 3H), 3.70-3.41 (m, 4H), 3.21 (br s, 4H), |
| | | | 2.77 (br s, 1H), 2.69 (d, J = 4.6 Hz, 3H), 2.65-2.55 (m, 2H), 2.42 (br s, |
| | | | 3H), 2.36-2.24 (m, 1H), 2.21-2.11 (m, 1H), 2.09-1.94 (m, 2H), 1.57 |
| | | | (br d, J = 6.9 Hz, 10H), 1.52-1.41 (m, 2H) |
| 148 | 230 | m/z = | ¹H NMR (400 MHz, DMSO-d6): δ 10.87 (s, 1H), 9.03 (s, 1H), 8.68 (d, |
| | | 426.3 | J = 2.1 Hz, 1H), 8.28 (d, J = 1.9 Hz, 1H), 8.07 (s, 1H), 7.96 (br d, J = |
| | | [M/2 + H]⁺ | 4.6 Hz, 1H), 7.39 (br d, J = 6.3 Hz, 1H), 7.14-6.97 (m, 3H), 5.97 (br d, |
| | | | J = 3.9 Hz, 1H), 4.59 (s, 2H), 4.33 (dd, J = 5.1, 9.6 Hz, 1H), 4.23 (s, |
| | | | 3H), 3.71-3.45 (m, 4H), 3.24-3.02 (m, 2H), 3.00-2.77 (m, 4H), 2.75- |
| | | | 2.56 (m, 6H), 2.38-2.25 (m, 1H), 2.25-1.93 (m, 5H), 1.70-1.40 (m, |
| | | | 12H) |
| 149 | 231a | m/z = | ¹H NMR (400 MHz, DMSO-d6): δ 10.88 (s, 1H), 9.03 (br s, 1H), 8.69 |
| | | 855.47 | (d, J = 2.00 Hz, 1H), 8.30 (d, J = 2.40 Hz, 1H), 8.07 (s, 1H), 7.95 (d, J = |
| | | [M + H]⁺ | 4.40 Hz, 1H), 7.38-7.39 (m, 1H), 7.09 (s, 1H), 7.02 (d, J = 5.60 Hz, |
| | | | 2H), 6.87 (br s, 1H), 4.58-4.54 (m, 5H), 4.33-4.32 (m, 1H), 4.24 (s, |
| | | | 3H), 3.68 (br s, 1H), 3.10-3.05 (m, 4H), 2.83 (s, 3H), 2.62-2.61 (m, |
| | | | 9H), 2.30 (s, 1H), 2.18-2.11 (m, 1H), 1.77-1.75 (m, 3H), 1.57 (d, J = |
| | | | 6.80 Hz, 6H), 1.30-1.25 (m, 2H), 0.85-0.83 (m, 2H). |
| 150 | 231b | m/z = | ¹H NMR (400 MHz, DMSO-d6): δ 10.87 (s, 1H), 9.03 (s, 1H), 8.68 (d, |
| | | 855.5 | J = 2.40 Hz, 1H), 8.30 (d, J = 6.20 Hz, 1H), 8.07 (s, 1H), 7.95 (d, J = |
| | | [M + H]⁺ | 4.80 Hz, 1H), 7.39-7.38 (m, 1H), 7.09 (s, 1H), 7.01 (d, J = 5.20 Hz, |
| | | | 2H), 5.95 (s, 1H), 4.48-4.45 (m, 5H), 4.33-4.32 (m, 1H), 4.24 (s, 3H), |
| | | | 3.68 (brs, 1H), 3.20 (brs, 4H), 3.04-2.85 (m, 3H), 2.62-2.61 (m, 5H), |
| | | | 2.40-2.38 (m, 3H), 2.28-2.18 (m, 1H), 2.07 (s, 1H), 1.84-1.81 (m, 4H), |
| | | | 1.64 (d, J = 14.80 Hz, 6H), 1.36 (s, 1H), 1.05 (brs, 2H). |
| 151 | 232a | m/z = | ¹H NMR (400 MHz, DMSO-d6): δ 10.85 (s, 1H), 9.03 (s, 1H), 8.69 (d, |
| | | 855.6 | J = 2.40 Hz, 1H), 8.30 (d, J = 2.40 Hz, 1H), 8.07 (s, 1H), 7.94 (d, J = |
| | | [M + H]⁺ | 4.40 Hz, 1H), 7.50 (d, J = 8.80 Hz, 1H), 7.08 (s, 1H), 6.89 (d, J = 9.20 |
| | | | Hz, 1H), 6.81 (s, 1H), 5.95 (br s, 1H), 4.57 (s, 3H), 4.46-4.44 (m, 2H), |
| | | | 4.27-4.24 (m, 1H), 3.89 (s, 3H), 3.64 (d, J = Hz, 1H), 3.50-3.40 (m, |
| | | | 3H), 2.97-2.84 (m, 5H), 2.67-2.59 (m, 6H), 2.41 (m, 2H), 2.38 (br, 1H), |
| | | | 2.35-2.33 (m, 2H), 1.79-1.69 (m, 3H), 1.56 (d, J = 6.80 Hz, 6H), 1.03 |
| | | | (m, 2H). |
| 152 | 233a | m/z = | ¹H NMR (400 MHz, DMSO-d6): δ 10.87 (s, 1H), 8.43 (d, J = 2.40 Hz, |
| | | 854.48 | 1H), 8.18 (s, 1H), 7.93-7.92 (m, 3H), 7.36-7.35 (m, 1H), 7.21 (s, 1H), |
| | | [M + H]⁺ | 7.02-7.00 (m, 2H), 6.22 (s, 1H), 6.00 (s, 1H), 4.51-4.52 (m, 3H), 4.32- |
| | | | 4.31 (m, 1H), 4.23 (s, 3H), 3.80-3.72 (m, 1H), 3.62-3.61 (m, 1H), 3.48- |
| | | | 3.42 (m, 1H), 3.15-3.12 (m, 4H), 3.01-2.96 (m, 1H), 2.84-2.81 (m, 1H), |
| | | | 2.62-2.60 (m, 8H), 2.20-2.18 (m, 5H), 2.00-1.91 (m, 1H), 1.89-1.81 (m, |
| | | | 1H), 1.55 (d, J = 11.20 Hz, 6H), 1.39-1.36 (m, 2H), 1.24 (s, 1H). |
| 170 | 197a | m/z = | 1H NMR 400 MHz, DMSO-d6: δ 10.89 (s, 1H), 9.12 (s, 1H), 8.72 (d, |
| | | 853.51 | J = 2.40 Hz, 1H), 8.57 (s, 1H), 8.26 (d, J = 2.00 Hz, 1H), 8.11 (s, 1H), |
| | | [M + H]+ | 7.96-7.97 (m, 1H), 7.49 (d, J = 8.00 Hz, 1H), 6.95-7.05 (m, 3H), 5.95 |
| | | | (br s, 1H), 4.60-4.45 (m, 4H), 4.40-4.30 (m, 1H), 4.26 (s, 3H), 3.81 (s, |
| | | | 1H), 3.72 (s, 2H), 3.30-3.00 (m, 4H), 2.90-2.80 (m, 4H), 2.64 (d, J = |
| | | | 5.60 Hz, 3H), 2.50-2.50 (m, 1H), 2.31 (s, 1H), δ 2.00-2.14 (m, 1H), |
| | | | 1.89 (d, J = 11.20 Hz, 2H), 1.58 (d, J = 6.80 Hz, 6H), 1.24-1.26 (m, |
| | | | 9H). |
| 171 | 210a | m/z = | 1H NMR (400 MHz, DMSO-d6): δ 10.90 (d, J = 4.40 Hz, 1H), 9.66 (s, |
| | | 823.48 | 1H), 9.35 (brs, 1H), 8.81 (d, J = 2.00 Hz, 1H), 8.31 (d, J = 2.40 Hz, |
| | | [M + H]+ | 1H), 8.10 (d, J = 3.60 Hz, 1H), 7.96 (d, J = 4.40 Hz, 1H), 7.58 (dd, J = |
| | | | 2.80, 8.00 Hz, 1H), 7.26-6.96 (m, 2H), 5.95 (brs, 1H), 4.59 (s, 2H), 4.50 |
| | | | (d, J = 12.40 Hz, 2H), 4.37 (q, J = 5.20 Hz, 1H), 4.28 (s, 3H), 3.61-3.52 |
| | | | (m, 3H), 3.38-3.22 (m, 2H), 3.17-3.09 (m, 3H), 3.03-2.88 (m, 3H), |
| | | | 2.68-2.61 (m, 5H), 2.33-2.32 (m, 1H), 2.20-2.17 (m, 2H), 1.82-1.80 (m, |
| | | | 2H), 1.57 (d, J = 6.80 Hz, 6H), 1.23-1.20 (m, 2H), 0.87 (d, J = 6.00 Hz, |
| | | | 3H). |
| 172 | 210b | m/z = | 1H NMR, 400 MHz, DMSO-d6: δ 10.91 (s, 1H), 9.66 (s, 1H), 9.35 |
| | | 823.48 | (brs, 1H), 8.81 (d, J = 2.40 Hz, 1H), 8.31 (d, J = 2.40 Hz, 1H), 8.10 (d, J = |
| | | [M + H]+ | 3.60 Hz, 1H), 7.96 (d, J = 4.40 Hz, 1H), 7.58 (dd, J = 2.80, 8.00 Hz, |
| | | | 1H), 7.27-7.20 (m, 1H), 7.17-7.10 (m, 1H), 6.01 (s, 1H), 4.59 (s, 2H), |
| | | | 4.50 (d, J = 12.40 Hz, 2H), 4.37 (q, J = 5.20 Hz, 1H), 4.28 (s, 3H), 3.59- |
| | | | 3.51 (br, 3H), 3.35-3.30 (m, 2H), 3.19-3.09 (m, 3H), 3.05-2.89 (m, 3H), |
| | | | 2.70-2.62 (m, 5H), 2.39-2.51 (m, 1H), 2.23-2.14 (m, 2H), 1.83-1.75 (m, |
| | | | 2H), 1.57 (d, J = 6.80 Hz, 6H), 1.25-1.19 (m, 2H), 0.87 (d, J = 6.00 Hz, |
| | | | 3H), |
| 173 | 224 | m/z = | 1H NMR (400 MHz, DMSO-d6): δ 10.89 (s, 1H), 9.11 (s, 1H), 8.66 (d, |
| | | 839.36 | J = 3.20 Hz, 1H), 8.30 (d, J = 2.00 Hz, 1H), 8.12 (s, 1H), 7.94 (d, J = |
| | | [M + H]+ | 4.80 Hz, 1H), 7.55 (d, J = 8.40 Hz, 1H), 7.25 (d, J = 8.40 Hz, 1H), 7.16 |
| | | | (s, 1H), 7.08 (t, J = 8.40 Hz, 1H), 5.95 (br s, 1H), 4.57 (s, 2H), 4.37- |
| | | | 4.23 (m, 1H), 4.20 (s, 2H), 3.81-3.75 (m, 2H), 3.60-3.50 (m, 4H), 3.25 |
| | | | (br s, 3H), 3.05-2.95 (m, 2H), 2.59-2.70-2.55 (m, 5H), 2.40-2.25 (m, |
| | | | 1H), 2.20-2.10 (m, 1H), 1.90-1.80 (m, 2H), 1.75-1.69 (m, 2H), 1.57- |
| | | | 1.56 (m, 6H), 1.25-1.23 (s, 2H), 0.85 (s, 1H). |
| 174 | 235 | m/z = | 1H NMR (400 MHz, DMSO-d6): δ 10.80 (s, 1H), 9.02 (s, 1H), 8.68 (d, |
| | | 839.52 | J = 2.40 Hz, 1H), 8.43 (brs, 2H), 8.28 (d, J = 2.40 Hz, 1H), 8.06 (s, 1H), |
| | | [M + H]+ | 7.94 (d, J = 4.80 Hz, 1H), 7.42 (d, J = 9.20 Hz, 1H), 7.08 (s, 1H), 6.69 |
| | | | (d, J = 1.60 Hz, 1H), 6.51 (s, 1H), 5.95 (s, 1H), 4.57 (s, 2H), 4.42 (d, J = |
| | | | 12.00 Hz, 2H), 4.21-4.20 (m, 1H), 3.83 (s, 3H), 3.54-3.53 (m, 3H), |
| | | | 2.77-2.74 (m, 4H), 2.67-2.66 (m, 3H), 2.60-2.59 (m, 2H), 2.45-2.44 (m, |
| | | | 1H), 2.28-2.25 (m, 2H), 2.16-2.14 (m, 1H), 1.89 (brs, 2H), 1.75-1.65 |
| | | | (m, 2H), 1.57 (d, J = 12.00 Hz, 6H), 1.35 (s, 2H) 0.99 (m, 2H). |
| 175 | 236 | m/z = | 1H NMR (400 MHz, DMSO-d6): 10.87 (s, 1H), 9.03 (s, 1H), 8.69 (d, J = |
| | | 839.52 | 2.40 Hz, 1H), 8.28 (d, J = 2.40 Hz, 1H), 8.07 (s, 1H), 7.94 (d, J = 4.40 |
| | | [M + H]+ | Hz, 1H), 7.37 (d, J = 7.60 Hz, 1H), 7.11-7.09 (m, 2H), 7.00-6.99 (t, J = |
| | | | 7.60 Hz, 1H), 5.96 (s, 1H), 4.57 (s, 2H), 4.46 (d, J = 11.60 Hz, 2H), |
| | | | 4.32-4.31 (m, 1H), 4.26 (s, 3H), 3.20 (brs, 4H), 2.77-2.75 (m, 5H), |
| | | | 2.70-2.61 (m, 5H), 2.32-2.31 (m, 3H), 2.18-2.17 (m, 1H), 1.92-1.89 (m, |
| | | | 2H), 1.79-1.76 (m, 3H), 1.58 (d, J = 8.80 Hz, 6H), 1.46 (s, 1H), 1.10- |
| | | | 1.03 (m, 2H). |
| 176 | 221c | m/z = | 1H NMR (400 MHz, DMSO-d6): δ = 10.83 (s, 1H), 9.06 (s, 1H), 8.79 |
| | | 850.4 | (d, J = 1.9 Hz, 1H), 8.45 (br s, 1H), 8.09 (s, 1H), 8.00 (br s, 1H), 7.33 |
| | | [M + H]+ | (d, J = 8.4 Hz, 1H), 7.15 (s, 1H), 6.57-6.35 (m, 2H), 6.16-5.92 (m, |
| | | | 1H), 5.75 (br d, J = 9.8 Hz, 1H), 4.63 (br s, 2H), 4.18 (dd, J = 5.1, 8.8 |
| | | | Hz, 1H), 3.94-3.85 (m, 1H), 3.82 (s, 3H), 3.79-3.62 (m, 4H), 2.68 (d, |
| | | | J = 4.5 Hz, 3H), 2.64-2.52 (m, 3H), 2.47-2.47 (m, 1H), 2.49-2.44 |
| | | | (m, 1H), 2.35-2.19 (m, 2H), 2.19-2.09 (m, 1H), 2.03 (br d, J = 10.4 |
| | | | Hz, 1H), 1.93 (br d, J = 11.3 Hz, 1H), 1.80-1.63 (m, 2H), 1.58 (d, J = |
| | | | 6.9 Hz, 6H), 1.39-1.19 (m, 1H) |
| 177 | 221d | m/z = | 1H NMR (400 MHz, DMSO-d6): δ = 10.82 (s, 1H), 9.05 (s, 1H), 8.88- |
| | | 850.5 | 8.70 (m, 1H), 8.07 (s, 1H), 8.02-7.91 (m, 1H), 7.34 (d, J = 8.8 Hz, |
| | | [M + H]+ | 1H), 7.25-7.15 (m, 1H), 6.68 (d, J = 8.0 Hz, 1H), 6.49 (s, 1H), 6.03- |
| | | | 5.88 (m, 1H), 5.88-5.74 (m, 1H), 4.59 (s, 2H), |
| | | | 4.21-4.06 (m, 2H), 3.87-3.79 (m, 4H), 3.77 (br d, J = 8.0 Hz, 2H), |
| | | | 3.74-3.68 (m, 1H), 2.68 (d, J = 4.8 Hz, 4H), 2.64-2.56 (m, 2H), 2.42- |
| | | | 2.22 (m, 3H), 2.22-2.01 (m, 2H), 1.98-1.89 (m, 1H), 1.88-1.78 (m, |
| | | | 2H), 1.74-1.63 (m, 1H), 1.57 (d, J = 6.8 Hz, 6H) |
| 178 | 232b | m/z = | 1H NMR (400 MHz, DMSO-d6): δ 9.03 (s, 1H), 8.69 (d, J = 2.40 Hz, |
| | | 855.5 | 1H), 8.30 (s, 1H), 8.07 (s, 1H), 7.95 (d, J = 4.80 Hz, 1H), 7.50 (d, J = |
| | | [M + H]+ | 8.80 Hz, 1H), 7.08 (s, 1H), 6.89 (d, J = 9.20 Hz, 1H), 6.81 (s, 1H), 6.00 |
| | | | (s, 1H), 4.57 (s, 2H), 4.46 (d, J = 10.80 Hz, 1H), 4.25-4.24 (m, 1H), |
| | | | 3.89 (s, 3H), 3.50-3.35 (m, 4H), 2.86-2.84 (m, 3H), 2.66 (d, J = 4.40 |
| | | | Hz, 3H), 2.61-2.51 (m, 2H), 2.41 (brs, 2H), 2.40-2.20 (m, 2H), 2.17- |
| | | | 2.13 (m, 2H), 1.86 (s, 6H), 1.72-1.69 (m, 2H), 1.56 (d, J = 6.80 Hz, |
| | | | 6H), 1.03 (m, 2H). |
| 179 | 198 | | 1H NMR (400 MHz, DMSO-d6): δ = 10.88 (s, 1H), 9.02 (s, 1H), 8.78 |
| | | | (d, J = 2.3 Hz, 1H), 8.58-8.38 (m, 1H), 8.06 (s, 1H), 7.97 (br d, J = 4.8 |
| | | | Hz, 1H), 7.12 (s, 1H), 6.64 (dd, J = 8.3, 10.1 Hz, 1H), 6.48 (dd, J = 3.9, |
| | | | 8.4 Hz, 1H), 6.06-5.87 (m, 1H), 4.81 (br d, J = 6.8 Hz, 1H), 4.59 (s, |
| | | | 2H), 4.28-4.22 (m, 3H), 3.79-3.64 (m, 4H), 3.22 (br s, 1H), 2.83- |
| | | | 2.63 (m, 5H), 2.56 (br d, J = 4.5 Hz, 1H), 2.33-2.17 (m, 1H), 2.17- |
| | | | 2.07 (m, 1H), 1.92 (br d, J = 10.5 Hz, 4H), 1.68-1.51 (m, 8H), 1.49- |
| | | | 1.35 (m, 2H). |
| 180 | 193b | m/z = | 1H NMR (400 MHz, DMSO-d6): δ 10.89 (s, 1H), 9.09 (s, 1H), 9.05 (br |
| | | 839.44 | s, 1H), 8.73 (d, J = 2.4 Hz, 1H), 8.27 (d, J = 2.4 Hz, 1H), 8.10 (s, 1H), |
| | | [M + H]+ | 7.96-7.95 (d, J = 4.4 Hz, 1H), 7.50-7.48 (d, J = 7.6 Hz, 1H), 7.11-7.06 |
| | | | (m, 3H), 5.95 (br s, 1H), 4.58 (s, 4H), 4.36-4.34 (m, 1H), 4.25 (s, 3H), |
| | | | 3.70-3.50 (m, 2H), 3.40-3.25 (m, 5H) 3.15 (s, 2H), 2.82 (t, J = 12 Hz, |
| | | | 2H), 2.60-2.50 (m, 5H), 2.25-2.16 (m, 3H), 1.75-1.56 (m, 7H), 1.35- |
| | | | 1.26 (m, 5H). |
| 181 | 195b | m/z = | 1H NMR (400 MHz, DMSO-d6): δ 10.88 (s, 1H), 9.06 (s, 1H), 8.67 (s, |
| | | 837.31 | 1H), 8.31 (s, 1H), 8.18 (s, 1H), 7.95 (d, J = 4.4 Hz, 1H), 7.37 (d, J = 6.8 |
| | | [M + H]+ | Hz, 1H), 7.15 (s, 1H), 7.01-6.99 (m, 2H), 5.96 (s, 1H), 4.58 (s, 2H), |
| | | | 4.34-4.31 (m, 1H), 4.24 (s, 3H), 3.61 (br s, 4H), 3.32 (s, 2H), 2.67-2.58 |
| | | | (m12H), 2.50-2.49 (m, 2H), 2.35-2.32 (m, 2H), 2.18-2.14 (m, 1H), |
| | | | 1.57-1.56 (m, 6H), 38-1.23 (m, 2H), 0.92-0.90 (m, 3H). |
| 182 | 201b | m/z = | 1H NMR (400 MHz, DMSO-d6): δ 10.84 (s, 1H), 9.02 (s 1H), 8.70 (d, |
| | | 852.45 | J = 2.4 Hz, 1H), 8.30 (d, J = 2.0 Hz, 1H), 8.07 (s, 1H), 7.94 (d, J = 4.4 |
| | | [M + H]+ | Hz, 1H), 7.46 (d, J = 8.8 Hz, 1H), 7.10 (s, 1H), 6.90 (d, J = 8.4 Hz, 1H), |
| | | | 6.80 (s, 1H), 5.95 (bs, 1H), 4.57-4.54 (m, 4H), 4.26-4.22 (m, 1H), 3.88- |
| | | | 3.84 (m, 6H), 3.76-2.59 (m, 9H), 2.30-2.28 (m, 1H), 2.18-2.14 (m, 1H), |
| | | | 1.81-1.55 (m, 14H), 1.35-1.20 (m, 2H), 0.97 (s, 3H). |

### Example P1

### Resolution of Stereoisomeric Mixtures

Mixtures of stereoisomers were resolved using methods described in Table P1.

| | | Retention | |
|---|---|---|---|
| Starting Material | Separation Method | Time | Final Product |
| tert-butyl 7-((3-(2,6- | Column: Chiralpak IH (30 × | R_{T} = 10.1 | 156a |
| bis(benzyloxy)pyridin-3- | 250 mm, internal diameter: 5 μm); | min | (Example 48) |
| yl)-1-methyl-1H-indazol- | mobile phase: Phase A (CO₂), and | R_{T} = 8.13 | 156b |
| 6-yl)amino)-6-methyl-2- | Phase B (MeOH with 0.2% | min | (Example 53) |
| azaspiro[3.5]nonane-2- | diethylamine); isocratic elution | R_{T} = 7.36 | 156c |
| carboxylate | B %: 50%; flow rate: 100 mL/min; | min | (Example 54) |
| | detector: PDA; wavelength: 225 | R_{T} = 10.7 | 156d |
| | nm; column temperature: 35° C.; | min | (Example 55) |
| | back pressure: 100 bar | | |
| tert-butyl 7-((3-(2,6- | Column: Lux-Cellulose -4 (30 × | R_{T} = 3.19 | 159a |
| bis(benzyloxy)pyridin-3- | 250 mm, internal diameter: 5 μm); | min | (Example 51) |
| yl)-1-methyl-1H-indazol- | mobile phase: Phase A (CO₂), and | R_{T} = 3.07 | 159b |
| 7-yl)amino)-6-methyl-2- | Phase B (ACN/MeOH with 0.2% | min | (Example 52) |
| azaspiro[3.5]nonane-2- | NH₃•H₂O); isocratic elution B %: | | |
| carboxylate | 40%; flow rate: 100 mL/min; | | |
| | detector: PDA; wavelength: 225 | | |
| | nm; column temperature: 30° C.; | | |
| | back pressure: 100 bar | | |
| tert-butyl 7-hydroxy-6- | Column: Chiralpak AD-3 (50 mm × | R_{T} = 1.28 | 185a |
| methyl-2- | 4.6 mm, 3 μm); mobile phase: | min | (Example 72) |
| azaspiro[3.5]nonane-2- | Phase A (CO₂), and Phase B | R_{T} = 1.04 | 185b |
| carboxylate | (iPrOH with 0.05% diethylamine); | min | (Example 73) |
| | Gradient elution: B in A from 5% | R_{T} = 1.16 | 185c |
| | to 40%; flow rate: 3 mL/min; | min | (Example 74) |
| | detector: PDA; wavelength: 220 | R_{T} = 1.27 | 185d |
| | nm; column temperature: 35° C.; | min | (Example162) |
| | back pressure: 100 bar | | |
| benzyl 4-(1-(1-(3-(2,6- | Column: Lux-Cellulose -4 (4.6 × | R_{T} = 5.37 | 194a |
| bis(benzyloxy)pyridin-3- | 150 mm, internal diameter: 3 μm); | min | (Example 76) |
| yl)-1-methyl-1H-indazol- | mobile phase: Phase A (CO₂), and | R_{T} = 6.47 | 194b |
| 7-yl)piperidin-4-yl)-1- | Phase B (ACN/MeOH with 0.2% | min | (Example 116) |
| hydroxyethyl)piperidine- | NH₃•H₂O); isocratic elution B %: | | |
| 1-carboxylate | 50%; flow rate: 3 mL/min; | | |
| | detector: PDA; wavelength: 230 | | |
| | nm; column temperature: 30° C.; | | |
| | back pressure: 100 bar | | |
| tert-butyl 7- | Column: Chiralpak AD-3 (50 mm × | R_{T} = 0.53 | 221a |
| (benzylamino)-6- | 4.6 mm, 3 μm); mobile phase: | min | (Example 78) |
| (trifluoromethyl)-2- | Phase A (CO₂), and Phase B | R_{T} = 0.85 | 221b |
| azaspiro[3.5]nonane-2- | (MeOH with 0.05% | min | (Example 141) |
| carboxylate | diethylamine); Gradient elution: B | R_{T} = 1.31 | 221c |
| | in A from 5% to 40%; flow rate: | min | (Example 176) |
| | 3 mL/min; detector: PDA; | R_{T} = 0.63 | 221d |
| | wavelength: 220 nm; column | | |
| | temperature: 35° C.; back pressure: | | |
| | 100 bar | min | (Example 177) |
| trans-tert-butyl-4-((4-(3- | Column: Lux-Cellulose -4 (4.6 × | R_{T} = 8.23 | 238a |
| (2-(benzyloxy)-6- | 150 mm, internal diameter: 3 μm); | min | (Example 163) |
| hydroxypyridin-3-yl)-1- | mobile phase: Phase A (CO₂), and | R_{T} = 10.20 | 238b |
| methyl-1H-indazol-7- | Phase B (ACN/MeOH with 0.2% | min | (Example 164) |
| yl)piperazin-1-yl)methyl)- | NH₃•H₂O); isocratic elution B %: | | |
| 3-fluoropiperidine-1- | 40%; flow rate: 3 mL/min; | | |
| carboxylate | detector: PDA; wavelength: 330 | | |
| | nm; column temperature: 30° C.; | | |
| | back pressure: 100 bar | | |
| cis-tert-butyl 4-((4-(3-(6- | Column: Chiralpak AD-H (4.6 × | R_{T} = 5.45 | 242a |
| (benzyloxy)-2- | 250 mm, internal diameter: 5 μm); | min | (Example 167) |
| hydroxypyridin-3-yl)-1- | mobile phase: Phase A (CO₂), and | R_{T} = 6.08 | 242b |
| methyl-1H-indazol-6- | Phase B (ACN/MeOH with 0.2% | min | (Example 168) |
| yl)piperidin-1-yl)methyl)- | 7N NH₃•H₂O); isocratic elution | | |
| 3-fluoropiperidine-1- | B %: 45%; flow rate: 3 mL/min; | | |
| carboxylate | detector: PDA; wavelength: 225 | | |
| | nm; column temperature: 30° C.; | | |
| | back pressure: 100 bar | | |

### Example B1

### HiBiT Based Degradation Assay

Human BCL6 protein coding open reading frame fused with N-terminal HiBiT coding sequence was synthesized from Integrated DNA Technologies (IDT). Next, the N-HiBiT-BCL6 sequence was cloned into pLV-UBC-PGK-Puro, a lentivirus plasmid purchased from Vectorbuilder, to generate pLV-UBC-N-HiBiT-BCL6-PGK-puro. Lentiviral particles were generated from Lenti-X™ 293T cells (Clontech) by co-transfection of pLV-UBC-N-HiBiT-BCL6-PGK-puro plasmids and lentiviral packaging plasmid mix (Cellecta). HT1080 [HT1080] (ATCC CCL-121) cells were infected with the lentivirus, and HT1080 cells stably integrated with the lentiviral vectors were established by incubation with 1 μg/mL puromycin (Thermofisher).

HT1080 cells expressing N-terminal HiBiT tagged BCL6 were dispensed into a 384-well plate pre-spotted with compounds at varying concentrations. Five thousand cells were seeded into each well in 40 μL of RPMI 1640 media plus 10% Fetal Bovine Serum (10082-147, Thermofisher). After 5 hours of incubation at 37° C. with 5% CO₂, 30 μL of the NANO-GLOR HiBiT Lytic Detection System working solution (Promega) was added to each well and incubated at room temperature for 15 min. After incubation, luminescence was read on a PHERAstar FSX Plate Reader (BMG). BCL6 degradation at each indicated concentration was normalized with DMSO control. The BCL6 degradation curves were plotted using a four-parameter logistic model.

Table B1 provides the Yₘᵢₙ and EC₅₀ values for certain compounds of this disclosure.

TABLE B1

| Compound No. | Yₘᵢₙ (5 hr) | EC₅₀ (5 hr) |
|---|---|---|
| 101 | ++++ | ++++ |
| 102 | ++++ | +++ |
| 103 | ++++ | +++ |
| 104 | +++ | ++++ |
| 105a | ++++ | +++ |
| 106a | ++ | ++++ |
| 107 | +++ | ++++ |
| 108 | ++ | +++ |
| 109 | +++ | +++ |
| 110 | +++ | +++ |
| 111 | +++ | ++++ |
| 112 | ++++ | ++++ |
| 113 | ++++ | +++ |
| 114 | +++ | ++++ |
| 115 | +++ | ++++ |
| 117 | ++++ | ++++ |
| 118 | ++++ | ++++ |
| 119 | +++ | ++++ |
| 120 | ++++ | ++++ |
| 121 | +++ | ++++ |
| 122a | ++ | +++ |
| 122b | +++ | +++ |
| 123a | ++ | ++++ |
| 123b | ++++ | ++++ |
| 132 | ++++ | ++++ |
| 133 | ++++ | ++++ |
| 134 | +++ | ++++ |
| 135 | ++++ | ++++ |
| 136a | ++++ | ++++ |
| 137 | ++++ | ++++ |
| 138 | ++++ | ++++ |
| 139 | +++ | ++ |
| 140 | +++ | ++++ |
| 141a | +++ | +++ |
| 142 | +++ | ++++ |
| 143 | ++++ | ++++ |
| 144 | ++++ | ++++ |
| 145 | ++++ | ++++ |
| 146a | ++++ | +++ |
| 146b | ++++ | +++ |
| 147 | ++++ | ++++ |
| 148 | +++ | ++++ |
| 149 | +++ | +++ |
| 150 | ++++ | ++++ |
| 151a | +++ | ++++ |
| 151b | ++ | ++++ |
| 152 | +++ | +++ |
| 153a | +++ | ++++ |
| 153b | +++ | ++++ |
| 154 | ++++ | ++++ |
| 155a | +++ | +++ |
| 155b | ++++ | ++++ |
| 156a | ++++ | ++++ |
| 156b | ++++ | ++++ |
| 156c | ++++ | ++++ |
| 156d | ++++ | ++++ |
| 157 | ++++ | +++ |
| 158 | ++ | ++++ |
| 159a | +++ | ++++ |
| 159b | +++ | ++++ |
| 160 | ++++ | ++++ |
| 161 | ++++ | +++ |
| 162 | ++++ | ++++ |
| 163a | +++ | ++ |
| 164 | +++ | ++++ |
| 165 | ++ | ++++ |
| 166 | +++ | ++++ |
| 167 | ++++ | ++++ |
| 168a | +++ | +++ |
| 168b | +++ | +++ |
| 169a | ++++ | ++++ |
| 169b | ++++ | ++++ |
| 170 | ++++ | +++ |
| 171 | ++ | ++++ |
| 172 | +++ | +++ |
| 173 | ++ | +++ |
| 174a | ++ | ++++ |
| 174b | +++ | +++ |
| 175a | +++ | ++++ |
| 175b | +++ | ++++ |
| 176a | +++ | ++++ |
| 176b | +++ | ++++ |
| 176c | +++ | +++ |
| 176d | +++ | ++++ |
| 177a | ++++ | +++ |
| 177b | +++ | +++ |
| 177c | +++ | ++ |
| 177d | ++++ | +++ |
| 178 | +++ | ++++ |
| 179a | +++ | ++ |
| 179b | +++ | ++ |
| 180a | +++ | +++ |
| 180b | +++ | ++++ |
| 180c | ++++ | ++++ |
| 180d | ++++ | ++++ |
| 181 | +++ | +++ |
| 182 | ++++ | ++++ |
| 183 | ++++ | ++++ |
| 184 | ++++ | ++++ |
| 185a | ++++ | ++++ |
| 185b | ++++ | ++++ |
| 185c | ++++ | ++++ |
| 185d | ++++ | ++++ |
| 186 | ++++ | +++ |
| 187 | +++ | +++ |
| 188a | +++ | +++ |
| 188b | ++++ | +++ |
| 189a | ++++ | +++ |
| 190 | ++ | +++ |
| 191 | +++ | +++ |
| 192a | ++++ | +++ |
| 192b | ++++ | +++ |
| 193a | +++ | +++ |
| 193b | ++++ | +++ |
| 194a | ++++ | +++ |
| 194b | +++ | +++ |
| 195a | ++++ | +++ |
| 195b | +++ | +++ |
| 196 | +++ | ++++ |
| 197a | +++ | +++ |
| 197b | ++++ | +++ |
| 198 | ++ | +++ |
| 199 | ++++ | ++++ |
| 200 | +++ | +++ |
| 201a | ++ | ++++ |
| 201b | +++ | ++++ |
| 202 | ++++ | ++++ |
| 203 | ++++ | ++++ |
| 204 | ++++ | +++ |
| 205 | ++++ | ++++ |
| 206 | ++++ | ++++ |
| 207 | +++ | ++++ |
| 208a | +++ | +++ |
| 208b | ++++ | +++ |
| 209 | +++ | ++++ |
| 210a | ++++ | +++ |
| 210b | ++++ | +++ |
| 211 | +++ | ++++ |
| 212 | ++++ | ++++ |
| 213 | ++++ | ++++ |
| 214 | +++ | ++++ |
| 215a | +++ | ++++ |
| 215b | +++ | +++ |
| 216a | ++ | +++ |
| 216b | +++ | +++ |
| 216c | ++ | ++++ |
| 216d | +++ | ++++ |
| 217a | ++++ | +++ |
| 217b | + | + |
| 218a | +++ | ++++ |
| 218b | +++ | ++++ |
| 219 | +++ | ++++ |
| 220 | +++ | ++++ |
| 221a | ++++ | ++++ |
| 221b | ++++ | ++++ |
| 222 | ++ | +++ |
| 223 | ++++ | ++++ |
| 224 | +++ | ++++ |
| 225 | ++++ | +++ |
| 226a | +++ | ++++ |
| 227a | ++ | ++++ |
| 228a | +++ | ++++ |
| 228b | ++ | +++ |
| 229 | ++++ | ++++ |
| 230 | +++ | +++ |
| 231a | ++++ | +++ |
| 231b | ++++ | ++++ |
| 232a | +++ | ++++ |
| 232b | +++ | ++++ |
| 233a | ++++ | +++ |
| 234 | ++ | ++ |
| 235 | +++ | +++ |
| 236 | +++ | ++++ |
| 237 | ++++ | ++++ |
| 238a | ++++ | +++ |
| 238b | ++++ | +++ |
| 239 | ++++ | +++ |
| 240 | ++++ | +++ |
| 241 | ++++ | +++ |
| 242a | ++++ | ++++ |
| 242b | ++++ | ++++ |

Notes:
Yₘᵢₙ < 30%: “++++”;
30% ≤ Yₘᵢₙ < 50%: “+++”;
50% ≤ Yₘᵢₙ < 70%; “++”;
Yₘᵢₙ ≥ 70%; “+”;
EC₅₀ < 10 nM: “++++”;
10 nM ≤ EC₅₀ < 100 nM: “+++”;
100 nM ≤ EC₅₀ < 1000 nM: “++”;
EC₅₀ ≥ 1000 nM: “+”

### Example B2

### Cell Proliferation Assay

OCI-Ly1 (DSMZ: ACC 722), SU-DHL-5 (ATCC CRL-2958), Toledo (ATCC CRL-2631), and WSU-DLCL2 (DSMZ: ACC 575) cells are seeded at 1,000 cells per well in 50 μL RPMI 1640 media plus 10% Fetal Bovine Serum (10082-147, Thermofisher) in black 384-well plates. The plates are pre-spotted with compounds at varying concentrations. After 5 days of incubation at 37° C. with 5% CO₂, cell viabilities are assessed using CELLTITER-GLO® Cell Viability Assay kit according to manufacturer's instructions (Promega). Relative cell proliferation at each concentration is normalized against DMSO control.

### REFERENCES

### OCI-Ly1 Cells:

1) Chang, H., Blondal, et al. (1995). Leukemia & Lymphoma 19 (1-2): 165-171.
2) Tweeddale, M. E., eta 1. (1987). Blood 69 (5): 1307-1314.
3) Farrugia, M. M., et al. (1994). Blood 83 (1): 191-198.
4) Mehra, S., et al. (2002). Genes Chromosomes Cancer 33 (3): 225-234.
5) Küppers, R., et al. (2003). Journal of Clinical Investigation 111 (4): 529-537.
6) Ngo, V. N., et al. (2011). Nature 470 (7332): 115-9.

### WSU-DLCL2 Cells:

1) Al-Katib, A. M., et al. (1998). Clinical Cancer Research 4 (5): 1305-1314.
2) Mohammad, R. M., et al. (2000). Clinical Cancer Research 6 (12): 4950-4956.
3) Morin, R. D., et al. (2010). Nature Genetics 42 (2): 181-185.
4) Quentmeier, H., et al. (2019). Scientific Reports 9 (1): 8218.

### Example B3

### Pharmacokinetic Assessment

Pharmacokinetics (PK) studies are conducted on male CD-1 mice by two delivery routes: intravenous (IV) injection and oral gavage (PO). Mice for the IV group (n=3) are allowed free access to food and water and mice for the PO group (n=3) are fasted 6-8 hours prior to dosing. The test articles are formulated in solution for the IV route (commonly 5% DMSO/10% Solutol HS15/85% water (pH 5)) and solution or suspension for the PO route (commonly 100% PEG400). On the day of the experiment, test article is administered via vein injection (commonly 1 mg/kg) for IV route or via oral gavage (commonly 10 mg/kg) for PO route, respectively. Blood samples are collected via the saphenous vein using a needle (commonly 20G) at 0.83 to 24 hours post dose. Approximately 30 μL of blood per timepoint is collected into pre-chilled tubes using K2EDTA as the anti-coagulant. After collection of blood samples at each time point, the blood samples are stored on ice. Blood samples are centrifuged within 0.5 hour of collection to separate plasma. Centrifugation is conducted at 2500×g for 15 minutes at 4° C. Plasma samples are immediately acidified (commonly 0.1M sodium citrate (pH=5) buffer). The samples are then submitted to LC-MS/MS for sample analysis. Pharmacokinetics parameters, including clearance (IV), area under the curve (AUC), and oral bioavailability (% F) are calculated by non-compartmental model.

### Exemplary Embodiments

### P01 Embodiments

Embodiment 1. A compound of Formula (I):

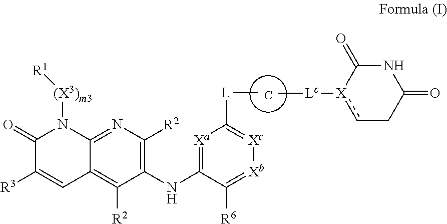

or a pharmaceutically acceptable salt thereof, wherein:
R¹ is selected from the group consisting of: H, halo, cyano, and R^{b1};
m3 is 0, 1, 2, or 3;
each X³ is independently selected from the group consisting of: —O—, —NR^{f}—, —C(═O)—, and C₁₋₃ alkylene optionally substituted with 1-3 R^{c};
provided that the N—(X³)ₘ₃—R¹ moiety does not contain any O—O, N—O, N—N, O-halo, or N-halo bonds;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e};
R³ is selected from the group consisting of:
-A¹-C(R⁴R⁴)-A² and —CH═CH-A², wherein:
A¹ is —O— or —S—;
each R⁴ is independently selected from the group consisting of: H, C₁₋₆ alkyl, and C₁₋₆ haloalkyl; or
the pair of R⁴ taken together with the carbon atom to which each is attached form a C₃₋₆ cycloalkyl ring or a 4-8 membered heterocyclyl ring, wherein the C₃₋₆ cycloalkyl ring or 4-8 membered heterocyclyl ring is optionally substituted with 1-3 R^{g};
A² is selected from the group consisting of: —C(O)OH, —C(O)NH₂, —C(O)R^{3A}, —C(O)OR^{3A}, —C(O)NR^{3A}R^{f}, —S(O)₁₋₂ (C₁₋₆ alkyl), —P(O)—(C₁₋₆ alkyl)₂, and —C(═NH) NH₂, wherein:
R^{3A} is selected from the group consisting of: C₁₋₆ alkyl, C₃₋₆ alkenyl, C₃₋₆ alkynyl, C₃₋₆ cycloalkyl, and 3-8 membered heterocyclyl, each of which is optionally substituted with 1-6 substituents independently selected from the group consisting of R^{a} and —(C₀₋₃ alkylene)-R^{b1};
X^{a} and X^{c} are independently selected from the group consisting of: N, CH, and CF, provided that one or both of X^{a} and X^{c} is N;
X^{b} is selected from the group consisting of N and CR^{x1};
R⁶ and R^{x1} are each independently selected from the group consisting of: H, halo, C₁₋₂ alkyl, C₁₋₂ haloalkyl, C₁₋₂ alkoxy, CN, and —C═CH;
L is -(L^{A})ₙ₁-, wherein L^{A} and n1 are defined according to (AA) or (BB):
   (AA)
n1 is an integer from 1 to 15; and
each L^{A} is independently selected from the group consisting of: L^{A1}, L^{A3}, and L^{A4}, provided that 1-3 occurrences of L^{A} is L^{A4};
   (BB)
n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, O—O, N—N, N—S(O) o, or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, S(O)₀₋₂ (C₁₋₆ alkyl), S(O)₀₋₂ (C₁₋₆ haloalkyl), S(O)₁₋₂N(R^{f})₂, —R^{b}, and C₁₋₆ alkyl optionally substituted with 1-6 R^{c};
Ring C is selected from the group consisting of:

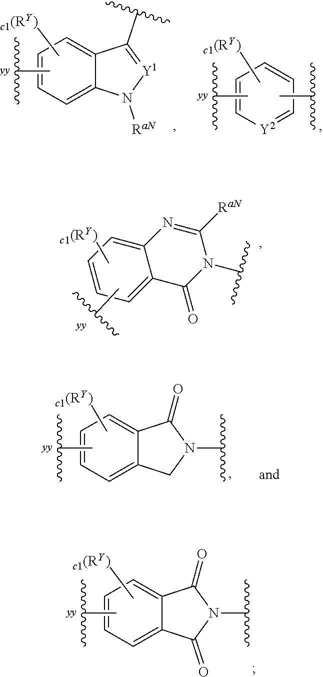

c1 is 0, 1, 2, or 3;
each R^{Y} is independently selected from the group consisting of: R^{a} and R^{b};
R^{aN} is H or C₁₋₆ alkyl optionally substituted with 1-3 R^{c};
Y¹ and Y² are independently N, CH, or CRY;
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —C(═O)—, —N(R^{d})—, and O, provided that when X is N, then L^{C} is other than O; and further provided that when Ring C is attached to -L^{C}- via a ring nitrogen, then X is CH, and L^{C} is a bond;
each R^{a} is independently selected from the group consisting of:
(a) halo;
(b) cyano;
(c) —OH;
(d) oxo;
(e) C₁₋₆ alkoxy optionally substituted with 1-6 R^{c};
(f) —NR^{d}R^{e};
(g) C(═O) C₁₋₆ alkyl optionally substituted with 1-6 R^{c};
(h) C(═O)OH;
(i) C(═O)OC₁₋₆ alkyl;
(j) C(═O)OC₁₋₆ haloalkyl;
(k) C(═O)N(R^{f})₂;
(l) S(O)₀₋₂ (C₁₋₆ alkyl);
(m) S(O)₀₋₂ (C₁₋₆ haloalkyl);
(n) S(O)₁₋₂N(R^{f}) 2; and
(o) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O) C₁₋₆ alkyl, C(═O) C₁₋₆ haloalkyl, C(═O)OC₁₋₆ alkyl, C(═O)OC₁₋₆ haloalkyl, C(═O)OH, C(═O)N(R^{f})₂, S(O)₀₋₂ (C₁₋₆ alkyl), S(O)₀₋₂ (C₁₋₆ haloalkyl), and S(O)₁₋₂N(R^{f})₂;
each R^{d} and R^{e} is independently selected from the group consisting of: H, C(═O) C₁₋₆ alkyl, C(═O) C₁₋₆ haloalkyl, C(═O)OC₁₋₆ alkyl, C(═O)OC₁₋₆ haloalkyl, C(═O)N(R^{f}) 2, S(O)₁₋₂ (C₁₋₆ alkyl), S(O)₁₋₂ (C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, cyano, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H) (C₁₋₃ alkyl), and —(C₀₋₃ alkylene)-N(C₁₋₃ alkyl)₂.

Embodiment 2. The compound of Embodiment 1, wherein R³ is -A¹-C(R⁴R⁴)-A².

Embodiment 3. The compound of Embodiments 1 or 2, wherein A¹ is —O—.

Embodiment 4. The compound of any one of Embodiments 1-3, wherein each R⁴ is H.

Embodiment 5. The compound of any one of Embodiments 1-3, wherein one R⁴ is C₁₋₃ alkyl (e.g., methyl); and the other R⁴ is H.

Embodiment 6. The compound of Embodiment 1, wherein R³ is —CH═CH-A².

Embodiment 7. The compound of any one of Embodiments 1-6, wherein A² is —C(O)NH₂ or —C(O)NR^{3A}R^{f}.

Embodiment 8. The compound of Embodiment 7, wherein R^{3A} is C₁₋₃ alkyl optionally substituted with 1-6 R^{c}.

Embodiment 9. The compound of Embodiments 1-8, wherein A² is —C(O)NH₂, —C(O)NHMe, or —C(O)NMe₂.

Embodiment 10. The compound of any one of Embodiments 1-9, wherein A² is —C(O)NHMe.

Embodiment 11. The compound of any one of Embodiments 1-4, wherein R³ is -A¹-C(R⁴R⁴)-A²; A¹ is O; each R⁴ is H; and A² is —C(O)NH₂ or —C(O)NR^{3A}R^{f}, wherein R^{3A} is C₁₋₃ alkyl optionally substituted with 1-6 R^{c}.

Embodiment 12. The compound of Embodiment 11, wherein A² is —C(O)NH₂, —C(O)NHMe, or —C(O)NMe₂.

Embodiment 13. The compound of any one of the Embodiments 1~4 or 11-12, wherein R³ is

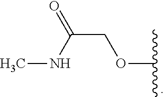 [SMILES: *OCC(=O)NC]

Embodiment 14. The compound of any one of Embodiments 1-13, wherein Xa is N; X^{c} is N; and X^{b} is CR^{x1} (e.g., CH).

Embodiment 15. The compound of any one of Embodiments 1-13, wherein Xa is CH; X^{c} is N; and X^{b} is CR^{x1} (e.g., CH).

Embodiment 16. The compound of any one of Embodiments 1-15, wherein R⁶ is —Cl or —F.

Embodiment 17. The compound of any one of Embodiments 1-16, wherein each R² is H.

Embodiment 18. The compound of any one of Embodiments 1-17, wherein m3 is 0.

Embodiment 19. The compound of any one of Embodiments 1-17, wherein m3 is 1; and X³ is C₁₋₃ alkylene (e.g., methylene, ethylene, or isopropylene).

Embodiment 20. The compound of any one of Embodiments 1-19, wherein R¹ is H.

Embodiment 21. The compound of any one of Embodiments 1-19, wherein R¹ is R^{b1}.

Embodiment 22. The compound of any one of Embodiments 1-19 or 21, wherein R¹ is C₃₋₆ cycloalkyl or 4-6 membered heterocyclyl, each of which is optionally substituted with 1-3 R^{g}.

Embodiment 23. The compound of Embodiment 22, wherein R¹ is selected from the group consisting of:

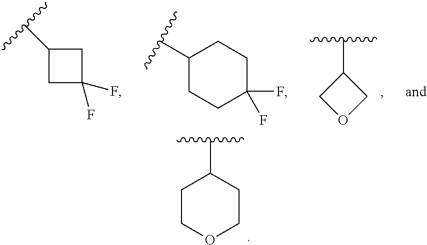

Embodiment 24. The compound of any one of Embodiments 1-17, wherein m3 is 1; X³ is methylene, ethylene, or isopropylene; and R¹ is H.

Embodiment 25. The compound of Embodiment 1, wherein:
R³ is -A¹-C(R⁴R⁴)-A², wherein A¹ is O; each R⁴ is H; and A² is —C(O)NH₂ or —C(O)NR^{3A}R^{f}, wherein R^{3A} is C₁₋₃ alkyl optionally substituted with 1-6 R^{c};
each R² is H;
X^{a} is N or CH;
X^{c} is N;
X^{b} is CH; and
R⁶ is —F or —C₁.

Embodiment 26. The compound of Embodiment 25, wherein m3 is 1; X³ is methylene, ethylene, or isopropylene; and R¹ is H.

Embodiment 27. The compound of Embodiments 25 or 26, wherein X^{a} is N.

Embodiment 28. The compound of any one of Embodiments 1-27, wherein Ring C is

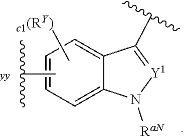

Embodiment 29. The compound of Embodiments 1-28, wherein Ring C is

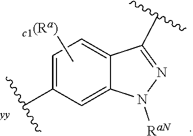

Embodiment 30. The compound of Embodiments 1-28, wherein Ring C is

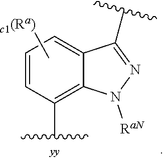

Embodiment 31. The compound of any one of Embodiments 1-27, wherein Ring C is

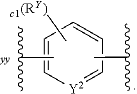

Embodiment 32. The compound of any one of Embodiments 1-27, wherein Ring C is selected from the group consisting of:

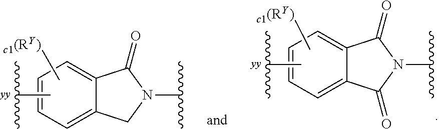

Embodiment 33. The compound of any one of Embodiments 1-32, wherein cl is 0.

Embodiment 34. The compound of any one of Embodiments 1-32, wherein cl is 1; and R^{Y} is halo (e.g., —F).

Embodiment 35. The compound of anyone of Embodiments 1-34, wherein X is CH.

Embodiment 36. The compound of any one of Embodiments 1-31 or 33-34, wherein X is N.

Embodiment 37. The compound of any one of Embodiments 1-30 or 33-35, wherein R^{aN} is C₁₋₃ alkyl (e.g., methyl).

Embodiment 38. The compound of any one of Embodiments 1-31, 33-35, or 37, wherein L^{C} is a bond.

Embodiment 39. The compound of any one of Embodiments 1-27, wherein the

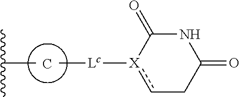

moiety is selected from the group consisting of:

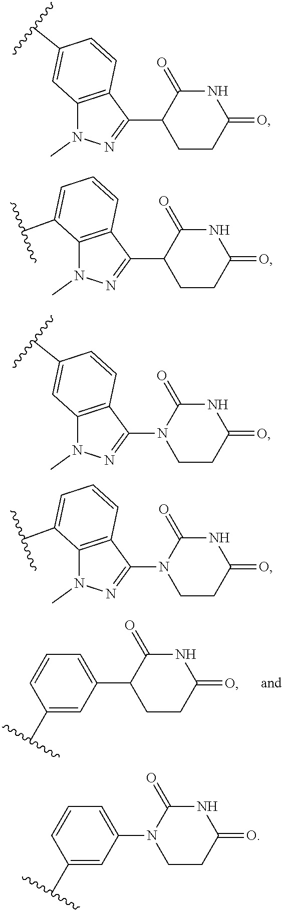

Embodiment 40. The compound of any one of Embodiments 1-27 or 39, wherein the

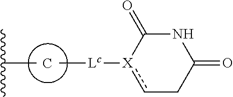

the moiety is selected from the group consisting of:

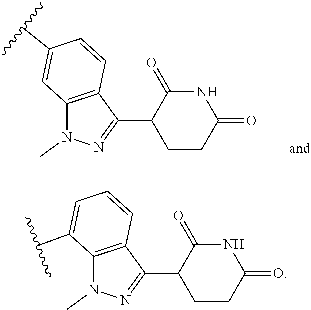

### Embodiment 41. The compound of any one of Embodiments 1-27, wherein the

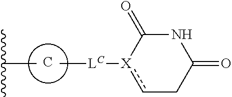

moiety is selected from the group consisting of:

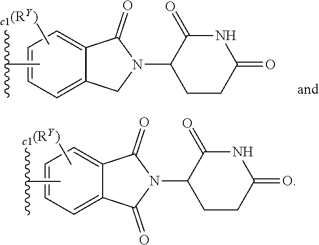

Embodiment 42. The compound of any one of Embodiments 1-27 or 41, wherein the

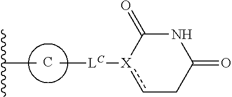

moiety is

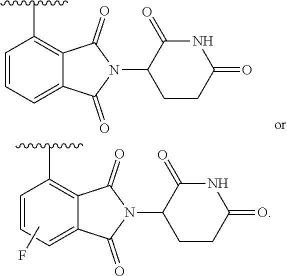

Embodiment 43. The compound of any one of Embodiments 1-42, wherein L is -(L^{A})ₙ₁—, wherein L^{A} and n1 are defined according to (AA).

Embodiment 44. The compound of any one of Embodiments 1-43, wherein n1 is an integer from 1 to 5.

Embodiment 45. The compound of any one of Embodiments 1-44, wherein n1 is an integer from 2 to 5 (e.g., 2 or 3).

Embodiment 46. The compound of any one of Embodiments 1-45, wherein L is selected from the group consisting of:

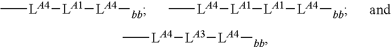

wherein bb represents the point of attachment to Ring C.

Embodiment 47. The compound of Embodiment 46, wherein each L^{A4} is independently a C₃₋₁₀ cycloalkylene or 4-12 membered heterocyclylene, each of which is optionally substituted with 1-6 R^{a}.

Embodiment 48. The compound of Embodiments 46 or 47, wherein each L^{A4} is independently a 4-12 membered heterocyclylene optionally substituted with 1-6 R^{a}.

Embodiment 49. The compound of any one of Embodiments 46-48, wherein each L^{A4} is independently a 4-10 membered heterocyclylene optionally substituted with 1-3 R^{a}.

Embodiment 50. The compound of any one of Embodiments 46-49, wherein each L^{A4} is independently a monocyclic 4-6 membered nitrogen-containing heterocyclylene optionally substituted with 1-3 R^{a}

Embodiment 51. The compound of any one of Embodiments 46-48, wherein one L^{A4} is a monocyclic 4-6 membered monocyclic nitrogen-containing heterocyclylene optionally substituted with 1-3 R^{a}; and the other L^{A4} is a bicyclic 6-12 membered nitrogen-containing heterocyclylene optionally substituted with 1-3 R^{a}.

Embodiment 52. The compound of Embodiment 51, wherein the other L^{A4} is a spirocyclic bicyclic 6-10 membered nitrogen-containing heterocyclylene optionally substituted with 1-3 R^{a}.

Embodiment 53. The compound of Embodiment 51, wherein the other L^{A4} is a bridged bicyclic 6-10 membered nitrogen-containing heterocyclylene optionally substituted with 1-3 R^{a}.

Embodiment 54. The compound of any one of Embodiments 47-53, wherein each R^{a} present on L^{A4} is independently selected from the group consisting of: F, methyl, CF₃, CHF₂, and CH₂F.

Embodiment 55. The compound of any one of Embodiments 46-54, wherein L is -L^{A4}-L^{A1}_L^{A4}-_{bb}, wherein bb represents the point of attachment to Ring C.

Embodiment 56. The compound of Embodiment 55, wherein L^{A1} is —CH₂—.

Embodiment 57. The compound of Embodiment 55, wherein L^{A1} is —C(Me)₂-.

Embodiment 58. The compound of any one of Embodiments 46-54, wherein L is -L^{A4}_L^{A3}-L^{A4}-_{bb}, wherein bb represents the point of attachment to Ring C.

Embodiment 59. The compound of Embodiment 58, wherein L^{A3} is —O—, —NH—, or —N(C₁₋₃ alkyl)-.

Embodiment 60. The compound of any one of Embodiments 46-54, wherein L is -L^{A4}_L^{A1}_L^{A1}_L^{A4}-_{bb}, wherein bb represents the point of attachment to Ring C.

Embodiment 61. The compound of Embodiment 60, wherein each L^{A1} is CH₂.

Embodiment 62. The compound of any one of Embodiments 1-45, wherein L is -L^{A4}_L^{A1}_(L^{A3})_{1-2-bb}, wherein bb represents the point of attachment to Ring C.

Embodiment 63. The compound of Embodiment 62, wherein L is -L^{A4}-L^{A1}-L^{A3}-_{bb}, wherein bb represents the point of attachment to Ring C.

Embodiment 64. The compound of Embodiments 62 or 63, wherein L^{A1} is —CH₂—

Embodiment 65. The compound of Embodiments 63 or 64, wherein L^{A3} is NH or N(C₁₋₃ alkyl) (e.g., NH).

Embodiment 66. The compound of any one of Embodiments 62-65, wherein L is -L^{A4}-CH₂—NH—_{bb}, wherein bb represents the point of attachment to Ring C.

Embodiment 67. The compound of any one of Embodiments 62-66, wherein L^{A4} is a 4-10 membered heterocyclylene optionally substituted with 1-3 R^{a}.

Embodiment 68. The compound of any one of Embodiments 62-67, wherein L^{A4} is a monocyclic 4-6 membered nitrogen-containing heterocyclylene optionally substituted with 1-3 R^{a}.

Embodiment 69. The compound of any one of Embodiments 1-46, wherein L is selected from the group consisting of the moieties delineated in Table L1a

Table L1a

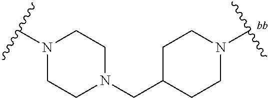

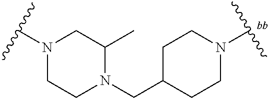

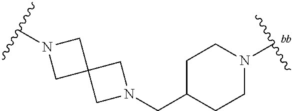

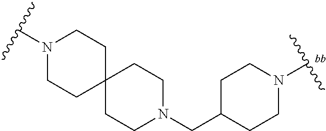

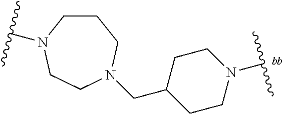

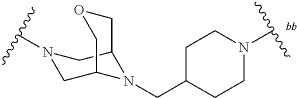

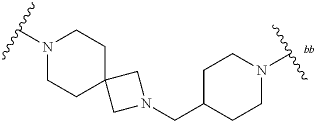

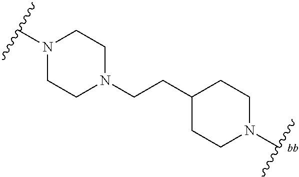

wherein bb represents the point of attachment to Ring C.

Embodiment 70. The compound of any one of Embodiments 1-45 or 62, wherein L is selected from the group consisting of the moieties delineated in Table L3a

TABLE L3a

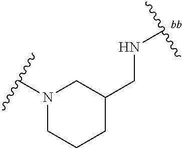

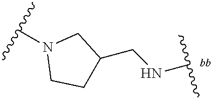

wherein bb represents the point of attachment to Ring C.

Embodiment 71. The compound of Embodiment 1, wherein:
the

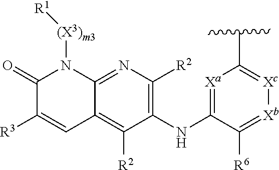

moiety is:

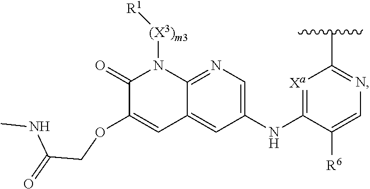

wherein:
R⁶ is —F or —Cl;
X^{a} is N or CH;
L is selected from the group consisting of:

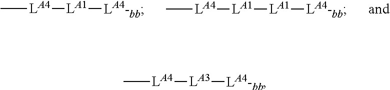

wherein bb represents the point of attachment to Ring C, wherein:
each L^{A4} is independently a monocyclic 4-6 membered nitrogen-containing heterocyclylene optionally substituted with 1-3 R^{a}; and
the

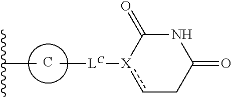

moiety is selected from the group consisting of:

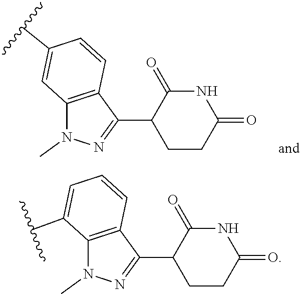

Embodiment 72. The compound of Embodiment 1, wherein:
the

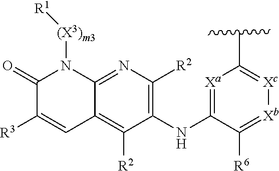

moiety is:

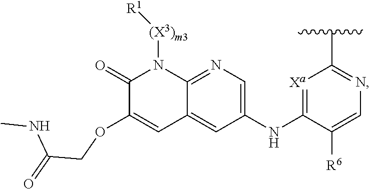

wherein:
R⁶ is —F or —C₁;
X^{a} is N or CH;
L is selected from the group consisting of:

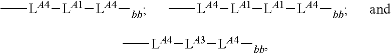

wherein bb represents the point of attachment to Ring C, wherein:
one L^{A4} is a monocyclic 4-6 membered nitrogen-containing heterocyclylene optionally substituted with 1-3 R^{a}; and the other L^{A4} is a bicyclic 6-12 membered nitrogen-containing heterocyclylene optionally substituted with 1-3 R^{a}; and
the

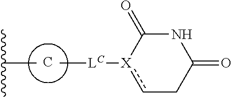

moiety is selected from the group consisting of:

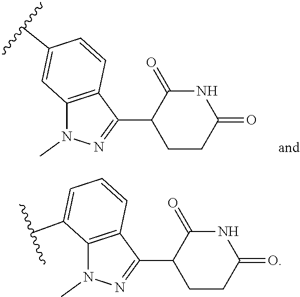

Embodiment 73. The compound of Embodiment 1, wherein:
the

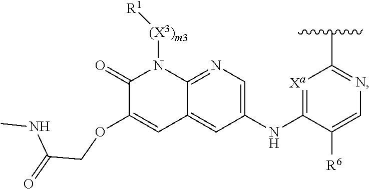

moiety is:

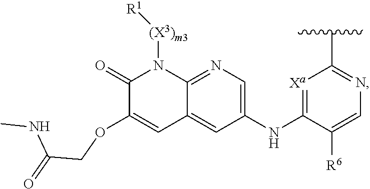

wherein:
R⁶ is —F or —C₁;
X^{a} is N or CH;
L is selected from the group consisting of the moieties delineated in Table L1a or Table L3a; and
the

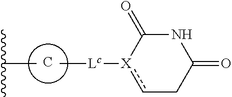

moiety is selected from the group consisting of:

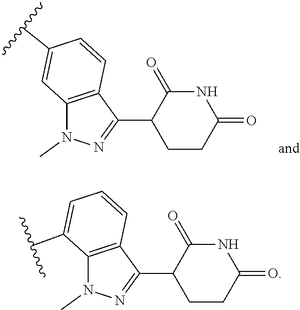

Embodiment 74. The compound of any one of Embodiments 71-73, wherein m3 is 1; X³ is methylene, ethylene, or isopropylene; and R¹ is H.

Embodiment 75. The compound of any one of Embodiments 71-74, wherein X^{a} is N.

Embodiment 76. The compound of Embodiment 1, wherein the compound is selected from the group consisting of Compound No. 101, 102, 103, 104, 105, 105a, 106, 106a, 107, 108, 109, 110, 111, 112, 113, 114, 115, 116, 117, 118, 119, 120, 121, 122, 122a, 122b, 123, 123a, 123b, 124, 125, 126, 126a, 127, 127a, 128, 129, 130, 130a, 131, and 131a as depicted in Table C1, or a pharmaceutically acceptable salt thereof.

Embodiment 77. A pharmaceutical composition comprising a compound of any one of Embodiments 1-76, or a pharmaceutically acceptable salt thereof, and a pharmaceutically acceptable excipient.

Embodiment 78. A BCL6 protein non-covalently bound with a compound of any one of Embodiments 1-76, or a pharmaceutically acceptable salt thereof.

Embodiment 79. A ternary complex comprising a BCL6 protein, a compound of any one of Embodiments 1-76, or a pharmaceutically acceptable salt thereof, and a CRBN protein.

Embodiment 80. A method for treating a cancer in a subject in need thereof, the method comprising administering to the subject a therapeutically effective amount of a compound of any one of Embodiments 1-76, or a pharmaceutically acceptable salt thereof, or a pharmaceutical composition according to Embodiment 77.

Embodiment 81. The method of Embodiment 80, wherein the cancer is a hematological cancer, breast cancer, gastrointestinal cancer, brain cancer, lung cancer, or a combination thereof.

Embodiment 82. The method of Embodiment 81, wherein the hematological cancer is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), chronic lymphocytic leukemia (CLL), acute lymphocytic leukemia (ALL), chronic myeloid leukemia (CML), or a combination thereof.

Embodiment 83. The method of Embodiment 82, wherein the hematological cancer is DLBCL.

Embodiment 84. The method of any one of Embodiments 80-83, further comprising administering an additional therapy or therapeutic agent to the subject.

Embodiment 85. The method of Embodiment 84, wherein the additional therapy or therapeutic agent is a PI3K inhibitor, a BTK inhibitor, a JAK inhibitor, a BRaf inhibitor, a MEK inhibitor, a Bcl-2 inhibitor, a Bcl-xL inhibitor, an XPO1 inhibitor, an immunomodulatory imide drug, anti-CD19 therapy, anti-CD20 therapy, anti-CD3 therapy, or a combination thereof.

Embodiment 86. A method for treating an autoimmune condition in a subject in need thereof, the method comprising administering to the subject a therapeutically effective amount of a compound of any one of Embodiments 1-76, or a pharmaceutically acceptable salt thereof, or a pharmaceutical composition according to Embodiment 77.

Embodiment 87. A method for treating a lymphoproliferative disorder in a subject in need thereof, the method comprising administering to the subject a therapeutically effective amount of a compound of any one of Embodiments 1-76, or a pharmaceutically acceptable salt thereof, or a pharmaceutical composition according to Embodiment 77.

Embodiment 88. A method for inducing degradation of a BCL6 protein in a mammalian cell, the method comprising contacting the mammalian cell with an effective amount of a compound of any one of Embodiments 1-76, or a pharmaceutically acceptable salt thereof.

Embodiment 89. The method of Embodiment 88, wherein the contacting occurs in vivo.

Embodiment 90. The method of Embodiment 88, wherein the contacting occurs in vitro.

Embodiment 91. The method of any one of Embodiments 88-90, wherein the mammalian cell is a mammalian cancer cell.

### P05 Embodiments

Embodiment 1. A compound of Formula (I):

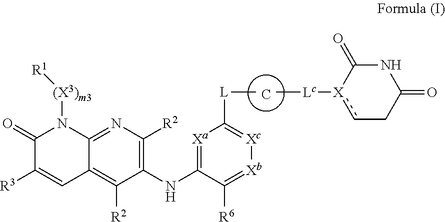

or a pharmaceutically acceptable salt thereof, wherein:
R¹ is selected from the group consisting of: H, halo, cyano, and R^{b1};
m3 is 0, 1, 2, or 3;
each X³ is independently selected from the group consisting of: —O—, —NR^{f}—, —C(═O)—, and C₁₋₃ alkylene optionally substituted with 1-3 R^{c};
provided that the N—(X³) m3-R¹ moiety does not contain any O—O, N—O, N—N, O-halo, or N-halo bonds;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e};
R³ is selected from the group consisting of:
-A¹-C(R⁴R⁴)-A² and —CH—CH-A², wherein:
A¹ is —O— or —S—;
each R⁴ is independently selected from the group consisting of: H, C₁₋₆ alkyl, and C₁₋₆ haloalkyl; or
the pair of R⁴ taken together with the carbon atom to which each is attached form a C₃₋₆ cycloalkyl ring or a 4-8 membered heterocyclyl ring, wherein the C₃₋₆ cycloalkyl ring or 4-8 membered heterocyclyl ring is optionally substituted with 1-3 R^{g};
A² is selected from the group consisting of: —C(O)OH, —C(O)NH₂, —C(O)R^{3A}, —C(O)OR^{3A}, —C(O)NR^{3A}R^{f}, —S(O)₁₋₂ (C₁₋₆ alkyl), —P(O)—(C₁₋₆ alkyl)₂, and —C(═NH) NH₂, wherein:
R^{3A} is selected from the group consisting of: C₁₋₆ alkyl, C₃₋₆ alkenyl, C₃₋₆ alkynyl, C₃₋₆ cycloalkyl, and 3-8 membered heterocyclyl, each of which is optionally substituted with 1-6 substituents independently selected from the group consisting of R^{a} and —(C₀₋₃ alkylene)-R^{b1};
X^{a} and X^{c} are independently selected from the group consisting of: N, CH, and CF, provided that one or both of X^{a} and X^{c} is N;
X^{b} is selected from the group consisting of N and CR^{x1};
R⁶ and R^{x1} are each independently selected from the group consisting of: H, halo, C₁₋₂ alkyl, C₁₋₂ haloalkyl, C₁₋₂ alkoxy, CN, and —C═CH;
L is -(L^{A})ₙ₁-, wherein L^{A} and n1 are defined according to (AA) or (BB):
   (AA)
n1 is an integer from 1 to 15; and
each L^{A} is independently selected from the group consisting of: L^{A1}, L^{A3}, and L^{A4}, provided that 1-3 occurrences of L^{A} is L^{A4}.
   (BB)
n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, O—O, N—N, N—S(O) o, or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f}) 2, S(O)₀₋₂ (C₁₋₆ alkyl), S(O)₀₋₂ (C₁₋₆ haloalkyl), S(O)₁₋₂N(R^{f})₂, —R^{b}, and C₁₋₆ alkyl optionally substituted with 1-6 R^{c};
Ring C is selected from the group consisting of:

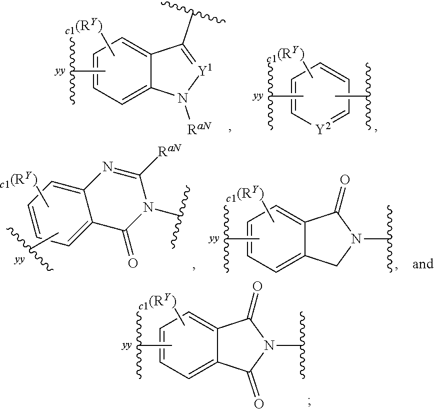

c1 is 0, 1, 2, or 3;
each R^{Y} is independently selected from the group consisting of: R^{a} and R^{b};
R^{aN} is H or C₁₋₆ alkyl optionally substituted with 1-3 R^{c};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —C(═O)—, —N(R^{d})—, and O, provided that when X is N, then L^{C} is other than O; and
further provided that when Ring C is attached to -L^{C}- via a ring nitrogen, then X is CH, and L^{C} is a bond;
each R^{a} is independently selected from the group consisting of:
(a) halo;
(b) cyano;
(c) —OH;
(d) oxo;
(e) C₁₋₆ alkoxy optionally substituted with 1-6 R^{c};
(f) —NR^{d}R^{e};
(g) C(═O) C₁₋₆ alkyl optionally substituted with 1-6 R^{c};
(h) C(═O)OH;
(i) C(═O)OC₁₋₆ alkyl;
(j) C(═O)OC₁₋₆ haloalkyl;
(k) C(═O)N(R^{f})₂;
(l) S(O)₀₋₂ (C₁₋₆ alkyl);
(m) S(O)₀₋₂ (C₁₋₆ haloalkyl);
(n) S(O)₁₋₂N(R^{f})₂; and
(o) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂ (C₁₋₆ alkyl), S(O)₀₋₂ (C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, cyano, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H) (C₁₋₃ alkyl), and —(C₀₋₃ alkylene)-N(C₁₋₃ alkyl) 2.

Embodiment 2. The compound of Embodiment 1, wherein R³ is -A¹-C(R⁴R⁴)-A².

Embodiment 3. The compound of Embodiments 1 or 2, wherein A¹ is —O—.

Embodiment 4. The compound of any one of Embodiments 1-3, wherein each R⁴ is H.

Embodiment 5. The compound of any one of Embodiments 1-3, wherein one R⁴ is C₁₋₃ alkyl (e.g., methyl); and the other R⁴ is H.

Embodiment 6. The compound of Embodiment 1, wherein R³ is —CH═CH-A².

Embodiment 7. The compound of any one of Embodiments 1-6, wherein A² is —C(O)NH₂ or —C(O)NR^{3A}R^{f}.

Embodiment 8. The compound of Embodiment 7, wherein R^{3A} is C₁₋₃ alkyl optionally substituted with 1-6 R^{c}.

Embodiment 9. The compound of Embodiments 1-8, wherein A² is —C(O)NH₂, —C(O)NHMe, or —C(O)NMe₂.

Embodiment 10. The compound of any one of Embodiments 1-9, wherein A² is —C(O)NHMe.

Embodiment 11. The compound of any one of Embodiments 1-4, wherein R³ is -A¹-C(R⁴R⁴)-A²; A¹ is O; each R⁴ is H; and A² is —C(O)NH₂ or —C(O)NR^{3A}R^{f}, wherein R^{3A} is C₁₋₃ alkyl optionally substituted with 1-6 R^{c}.

Embodiment 12. The compound of Embodiment 11, wherein A² is —C(O)NH₂, —C(O)NHMe, or —C(O)NMe₂.

Embodiment 13. The compound of any one of the Embodiments 1~4 or 11-12, wherein R³ is

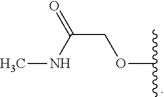 [SMILES: *OCC(=O)NC]

Embodiment 14. The compound of any one of Embodiments 1-13, wherein X^{a} is N; X^{c} is N; and X^{b} is CR^{x1} (e.g., CH).

Embodiment 15. The compound of any one of Embodiments 1-13, wherein X^{a} is CH; X^{c} is N; and X^{b} is CR^{x1} (e.g., CH).

Embodiment 16. The compound of any one of Embodiments 1-15, wherein R⁶ is —Cl or —F.

Embodiment 17. The compound of any one of Embodiments 1-16, wherein each R² is H.

Embodiment 18. The compound of any one of Embodiments 1-17, wherein m3 is 0.

Embodiment 19. The compound of any one of Embodiments 1-17, wherein m3 is 1; and X³ is C₁₋₃ alkylene (e.g., methylene, ethylene, or isopropylene).

Embodiment 20. The compound of any one of Embodiments 1-19, wherein R¹ is H.

Embodiment 21. The compound of any one of Embodiments 1-19, wherein R¹ is R^{b1}.

Embodiment 22. The compound of any one of Embodiments 1-19 or 21, wherein R¹ is C₃₋₆ cycloalkyl or 4-6 membered heterocyclyl, each of which is optionally substituted with 1-3 R^{g}.

Embodiment 23. The compound of Embodiment 22, wherein R¹ is selected from the group consisting of:

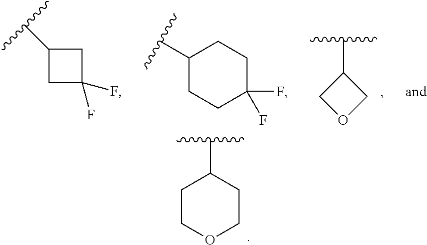

Embodiment 24. The compound of any one of Embodiments 1-17, wherein m3 is 1; X³ is methylene, ethylene, or isopropylene; and R¹ is H.

Embodiment 25. The compound of Embodiment 1, wherein:
R³ is -A¹-C(R⁴R⁴)-A², wherein A¹ is O; each R⁴ is H; and A² is —C(O)NH₂ or —C(O)NR^{3A}R^{f}, wherein R^{3A} is C₁₋₃ alkyl optionally substituted with 1-6 R^{c};
each R² is H;
X^{a} is N or CH;
X^{c} is N;
X^{b} is CH; and
R⁶ is —F or —Cl.

Embodiment 26. The compound of Embodiment 25, wherein m3 is 1; X³ is methylene, ethylene, or isopropylene; and R¹ is H.

Embodiment 27. The compound of Embodiments 25 or 26, wherein X^{a} is N.

Embodiment 28. The compound of any one of Embodiments 1-27, wherein Ring C is

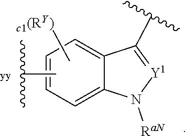

Embodiment 29. The compound of Embodiments 1-28, wherein Ring C is

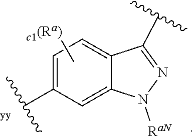

Embodiment 30. The compound of Embodiments 1-28, wherein Ring C is

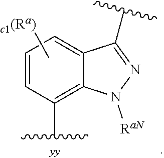

Embodiment 31. The compound of any one of Embodiments 1-27, wherein Ring C is

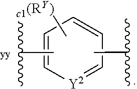

Embodiment 32. The compound of any one of Embodiments 1-27, wherein Ring C is selected from the group consisting of:

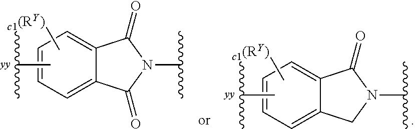

Embodiment 33. The compound of any one of Embodiments 1-32, wherein cl is 0.

Embodiment 34. The compound of any one of Embodiments 1-32, wherein cl is 1; and R^{Y} is halo (e.g., —F).

Embodiment 35. The compound of anyone of Embodiments 1-34, wherein X is CH.

Embodiment 36. The compound of any one of Embodiments 1-31 or 33-34, wherein X is N.

Embodiment 37. The compound of any one of Embodiments 1-30 or 33-35, wherein R^{aN} is C₁₋₃ alkyl (e.g., methyl).

Embodiment 38. The compound of any one of Embodiments 1-31, 33-35, or 37, wherein L^{C} is a bond.

Embodiment 39. The compound of any one of Embodiments 1-27, wherein the

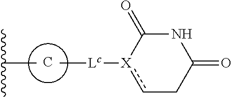

moiety is selected from the group consisting of:

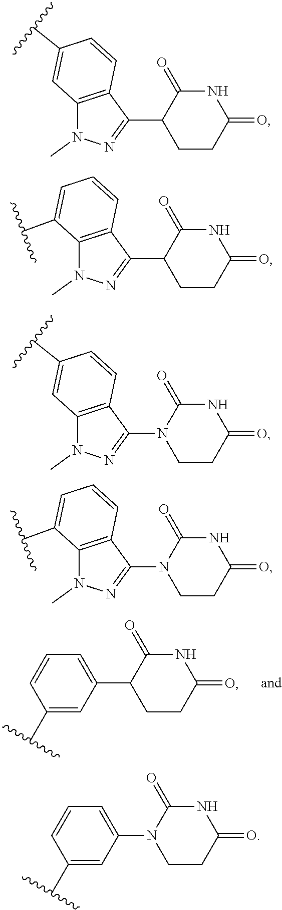

Embodiment 40. The compound of any one of Embodiments 1-27 or 39, wherein the

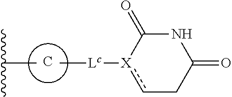

moiety is selected from the group consisting of:

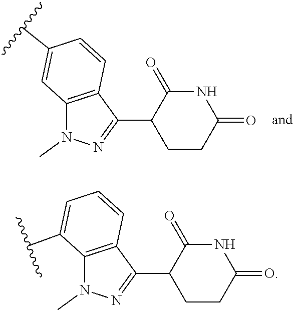

Embodiment 41. The compound of any one of Embodiments 1-27, wherein the

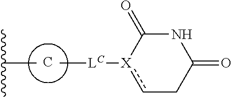

moiety is selected from the group consisting of:

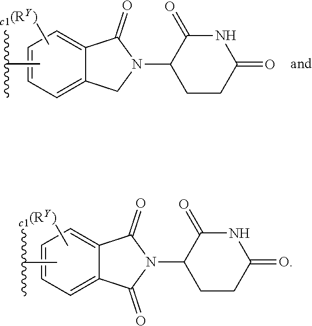

Embodiment 42. The compound of any one of Embodiments 1-27 or 41, wherein the

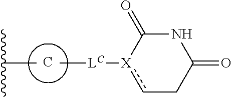

moiety is

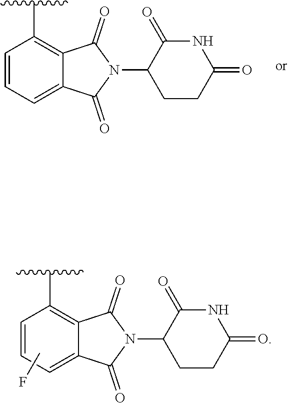

Embodiment 43. The compound of any one of Embodiments 1-42, wherein L is -(L^{A})ₙ₁—, wherein L^{A} and n1 are defined according to (AA).

Embodiment 44. The compound of any one of Embodiments 1-43, wherein n1 is an integer from 1 to 5.

Embodiment 45. The compound of any one of Embodiments 1-44, wherein n1 is an integer from 2 to 5 (e.g., 2 or 3).

Embodiment 46. The compound of any one of Embodiments 1-45, wherein L is selected from the group consisting of:

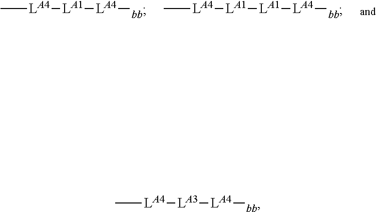

wherein bb represents the point of attachment to Ring C.

Embodiment 47. The compound of Embodiment 46, wherein each L^{A4} is independently a C₃₋₁₀ cycloalkylene or 4-12 membered heterocyclylene, each of which is optionally substituted with 1-6 R^{a}.

Embodiment 48. The compound of Embodiments 46 or 47, wherein each L^{A4} is independently a 4-12 membered heterocyclylene optionally substituted with 1-6 R^{a}.

Embodiment 49. The compound of any one of Embodiments 46-48, wherein each L^{A4} is independently a 4-10 membered heterocyclylene optionally substituted with 1-3 R^{a}.

Embodiment 50. The compound of any one of Embodiments 46-49, wherein each L^{A4} is independently a monocyclic 4-6 membered nitrogen-containing heterocyclylene optionally substituted with 1-3 R^{a}

Embodiment 51. The compound of any one of Embodiments 46-48, wherein one L^{A4} is a monocyclic 4-6 membered monocyclic nitrogen-containing heterocyclylene optionally substituted with 1-3 R^{a}; and the other L^{A4} is a bicyclic 6-12 membered nitrogen-containing heterocyclylene optionally substituted with 1-3 R^{a}.

Embodiment 52. The compound of Embodiment 51, wherein the other L^{A4} is a spirocyclic bicyclic 6-10 membered nitrogen-containing heterocyclylene optionally substituted with 1-3 R^{a}.

Embodiment 53. The compound of Embodiment 51, wherein the other L^{A4} is a bridged bicyclic 6-10 membered nitrogen-containing heterocyclylene optionally substituted with 1-3 R^{a}

Embodiment 54. The compound of any one of Embodiments 47-53, wherein each R^{a} present on L^{A4} is independently selected from the group consisting of: F, methyl, CF₃, CHF₂, and CH₂F.

Embodiment 55. The compound of any one of Embodiments 46-54, wherein L is -L^{A4}_L^{A1}-L^{A4}-_{bb}, wherein bb represents the point of attachment to Ring C.

Embodiment 56. The compound of Embodiment 55, wherein L^{A1} is —CH₂—.

Embodiment 57. The compound of Embodiment 55, wherein L^{A1} is —C(Me)₂-.

Embodiment 58. The compound of any one of Embodiments 46-54, wherein L is -L^{A4}_L^{A3}_L^{A4}-_{bb}, wherein bb represents the point of attachment to Ring C.

Embodiment 59. The compound of Embodiment 58, wherein L^{A3} is —O—, —NH—, or —N(C₁₋₃ alkyl)-.

Embodiment 60. The compound of any one of Embodiments 46-54, wherein L is -L^{A4}_L^{A1}-L^{A1}-L^{A4}-_{bb}, wherein bb represents the point of attachment to Ring C.

Embodiment 61. The compound of Embodiment 60, wherein each L^{A1} is CH₂.

Embodiment 62. The compound of any one of Embodiments 1-45, wherein L is -L^{A4}-L^{A1}_(L^{A3})_{1-2-bb}, wherein bb represents the point of attachment to Ring C.

Embodiment 63. The compound of Embodiment 62, wherein L is -L^{A4}-L^{A1}-L^{A3}-_{bb}, wherein bb represents the point of attachment to Ring C.

Embodiment 64. The compound of Embodiments 62 or 63, wherein L^{A1} is —CH₂—

Embodiment 65. The compound of Embodiments 63 or 64, wherein L^{A3} is NH or N(C₁₋₃ alkyl) (e.g., NH).

Embodiment 66. The compound of any one of Embodiments 62-65, wherein L is -L^{A4}-CH₂—NH—_{bb}, wherein bb represents the point of attachment to Ring C.

Embodiment 67. The compound of any one of Embodiments 62-66, wherein L^{A4} is a 4-10 membered heterocyclylene optionally substituted with 1-3 R^{a}.

Embodiment 68. The compound of any one of Embodiments 62-67, wherein L^{A4} is a monocyclic 4-6 membered nitrogen-containing heterocyclylene optionally substituted with 1-3 R^{a}.

Embodiment 69. The compound of any one of Embodiments 1-46, wherein L is selected from the group consisting of the moieties delineated in Table L1a

TABLE L1a

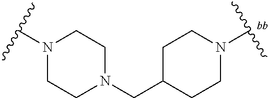

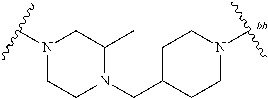

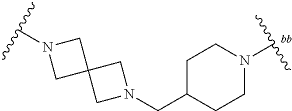

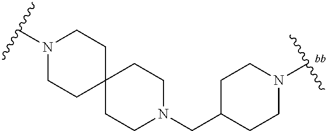

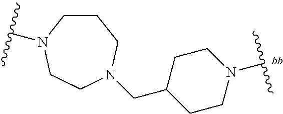

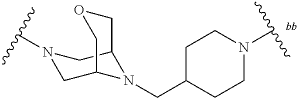

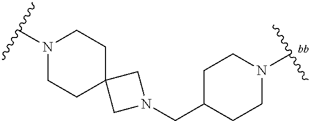

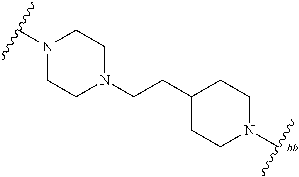

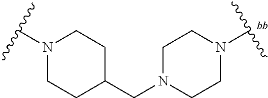

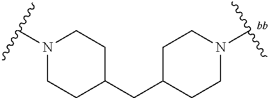

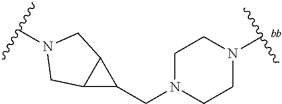

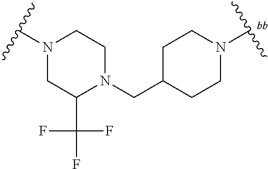

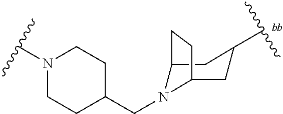

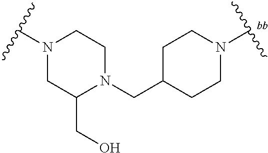

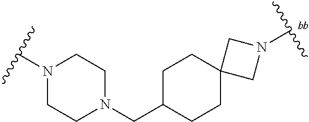

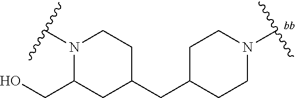

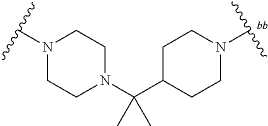

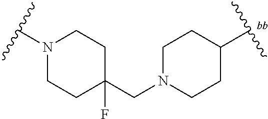

wherein bb represents the point of attachment to Ring C.

Embodiment 70. The compound of any one of Embodiments 1-45 or 62, wherein L is selected from the group consisting of the moieties delineated in Table L3a

TABLE L3a

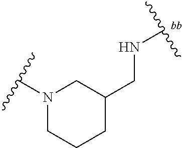

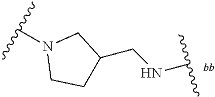

wherein bb represents the point of attachment to Ring C.

Embodiment 71. The compound of Embodiment 1, wherein:
the

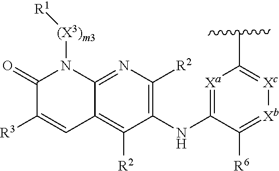

moiety is:

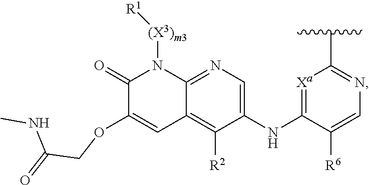

wherein:
R⁶ is —F or —C₁;
X^{a} is N or CH;
L is selected from the group consisting of:

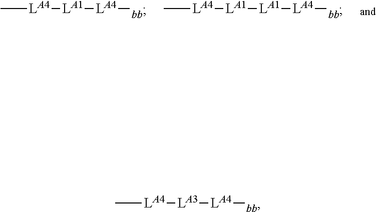

wherein bb represents the point of attachment to Ring C, wherein:
each L^{A4} is independently a monocyclic 4-6 membered nitrogen-containing heterocyclylene optionally substituted with 1-3 R^{a}; and
the

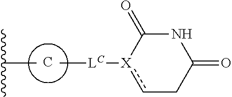

moiety is selected from the group consisting of:

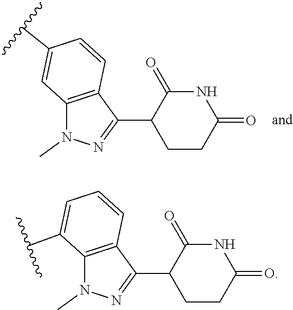

Embodiment 72. The compound of Embodiment 1, wherein:
the

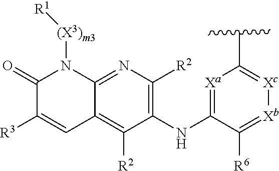

moiety is:

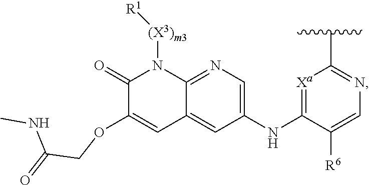

wherein:
R⁶ is —F or —C₁;
X^{a} is N or CH;
L is selected from the group consisting of:

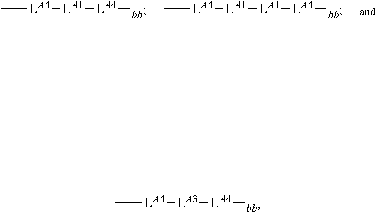

wherein bb represents the point of attachment to Ring C, wherein:

one L^{A4} is a monocyclic 4-6 membered nitrogen-containing heterocyclylene optionally substituted with 1-3 R^{a}; and the other L^{A4} is a bicyclic 6-12 membered nitrogen-containing heterocyclylene optionally substituted with 1-3 R^{a}; and
the

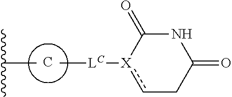

moiety is selected from the group consisting of:

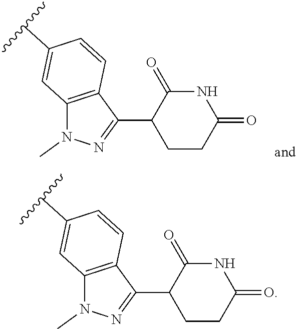

Embodiment 73. The compound of Embodiment 1, wherein:
the

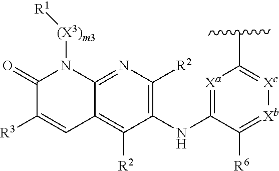

moiety is:

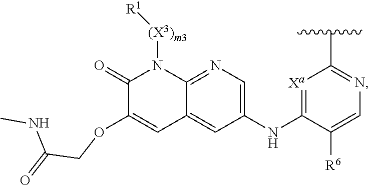

wherein:
R⁶ is —F or —C₁;
X^{a} is N or CH;
L is selected from the group consisting of the moieties delineated in Table L1a or Table L3a; and
the

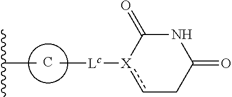

moiety is selected from the group consisting of:

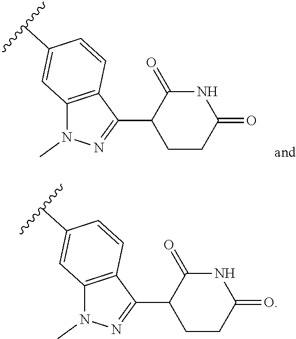 [SMILES: *c1ccc2c(C3CCC(=O)NC3=O)nn(C)c2c1 ; *c1ccc2c(C3CCC(=O)NC3=O)nn(C)c2c1]

Embodiment 74. The compound of any one of Embodiments 71-73, wherein m3 is 1; X³ is methylene, ethylene, or isopropylene; and R¹ is H.

Embodiment 75. The compound of any one of Embodiments 71-74, wherein X^{a} is N.

Embodiment 76. The compound of Embodiment 1, wherein the compound is selected from the group consisting of Compound No. 101, 102, 103, 104, 105, 105a, 106, 106a, 107, 108, 109, 110, 111, 112, 113, 114, 115, 116, 117, 118, 119, 120, 121, 122, 122a, 122b, 123, 123a, 123b, 124, 125, 126, 126a, 127, 127a, 128, 129, 130, 130a, 131, 131a, 132, 133, 134, 135, 136, 136a, 137, 138, 139, 140, 141, 141a, 142, 143, 144, 145, 146, 146a, 146b, 147, 148, 149, 150, 151, 151a, 151b, 152, 153, 153a, 153b, 154, 155, 155a, 155b, 156, 156a, 156b, 156c, 156d, 157, 158, 159, 159a, 159b, 160, 161, 162, 163, 163a, 164, 165, 166, 167, 168, 168a, 168b, 169, 169a, 169b, 170, 171, 172, 173, 174, 174a, 174b, 175, 175a, 175b, 176, 176a, 176b, 176c, 176d, 177, 177a, 177b, 177c, 177d, 178, 179, 179a, 179b, 180, 180a, 180b, 180c, 180d, 181, 182, 183, 184, 185, 185a, 185b, 185c, 185d, 186, 187, 188, 188a, 188b, 189, 189a, 190, 191, 192, 192a, 192b, 193, 193a, 194, 194a, 194b, 195, 195a, 196, 197, 197a, 197b, 198, 199, 200, 201, 201a, 202, 203, 204, 205, 206, 207, 208, 208a, 208b, 209, 210, 210a, 210b, 211, 212, 213, 214, 215, 215a, 215b, 216, 216a, 216b, 216c, 216d, 217, 217a, 217b, 218, 218a, 218b, 219, 220, 221, 221a, 221b, 221c, 221d, 222, 223, 224, 225, 226, 226a, 227, 227a, 228, 228a, 228b, 229, 230, 231, 231a, 231b, 232, 232a, 232b, 233, 233a, 235, 236, 237, 238, 238a, 238b, 239, 240, 241, 242, 242a, and 242b as depicted in Table C₁, or a pharmaceutically acceptable salt thereof.

Embodiment 77. A pharmaceutical composition comprising a compound of any one of Embodiments 1-76, or a pharmaceutically acceptable salt thereof, and a pharmaceutically acceptable excipient.

Embodiment 78. A BCL6 protein non-covalently bound with a compound of any one of Embodiments 1-76, or a pharmaceutically acceptable salt thereof.

Embodiment 79. A ternary complex comprising a BCL6 protein, a compound of any one of Embodiments 1-76, or a pharmaceutically acceptable salt thereof, and a CRBN protein.

Embodiment 80. A method for treating a cancer in a subject in need thereof, the method comprising administering to the subject a therapeutically effective amount of a compound of any one of Embodiments 1-76, or a pharmaceutically acceptable salt thereof, or a pharmaceutical composition according to Embodiment 77.

Embodiment 81. The method of Embodiment 80, wherein the cancer is a hematological cancer, breast cancer, gastrointestinal cancer, brain cancer, lung cancer, or a combination thereof.

Embodiment 82. The method of Embodiment 81, wherein the hematological cancer is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), peripheral T-cell lymphoma (PTCL), chronic lymphocytic leukemia (CLL), acute lymphocytic leukemia (ALL), or chronic myeloid leukemia (CML).

Embodiment 83. The method of Embodiment 82, wherein the hematological cancer is selected from the group consisting of DLBCL, FL, MCL, BL, PTCL, and ALL (e.g., B-ALL).

Embodiment 84. The method of Embodiment 82, wherein the hematological cancer is FL or DLBCL.

Embodiment 85. The method of Embodiment 82, wherein the hematological cancer is DLBCL.

Embodiment 86. The method of Embodiment 82, wherein the hematological cancer is FL.

Embodiment 87. The method of Embodiment 82, wherein the hematological cancer is BL.

Embodiment 88. The method of Embodiment 82, wherein the hematological cancer is a PTCL.

Embodiment 89. The method of Embodiment 82, wherein, the hematological cancer is ALL (e.g., B-ALL).

Embodiment 90. The method of any one of Embodiments 80-89, wherein the therapeutically effective amount of a compound of any one of Embodiments 1-76, or a pharmaceutically acceptable salt thereof, or a pharmaceutical composition according to Embodiment 77, is administered to the subject as a monotherapy.

Embodiment 91. The method of any one of Embodiments 80-89, comprising administering an additional therapy or therapeutic agent to the subject.

Embodiment 92. The method of Embodiment 91, wherein the additional therapy or therapeutic agent is a PI3K inhibitor, an Abl inhibitor (e.g., a BCR-Abl inhibitor), a BTK inhibitor, a JAK inhibitor, a BRaf inhibitor, a MEK inhibitor, a BCL-2 inhibitor, a BCL-XL inhibitor, an XPO1 inhibitor, an inhibitor of the polycomb repressive complex 2 (PRC2), an immunomodulatory imide drug, anti-CD19 therapy, anti-CD20 therapy, anti-CD3 therapy, chemotherapy, or a combination thereof.

Embodiment 93. The method of Embodiment 92, wherein the additional therapy or therapeutic agent is an Abl inhibitor.

Embodiment 94. The method of Embodiment 93, wherein the cancer is a B-ALL (e.g., a Philadelphia chromosome positive B-ALL or Philadelphia chromosome-like B-ALL).

Embodiment 95. The method of Embodiment 92, wherein the additional therapy or therapeutic agent is a BTK inhibitor.

Embodiment 96. The method of Embodiment 95, wherein the cancer is a B-ALL (e.g., a pre-BCR+B-ALL or a B-ALL dependent on Ras signaling).

Embodiment 97. The method of Embodiment 92, wherein the additional therapy or therapeutic agent is a JAK inhibitor.

Embodiment 98. The method of Embodiment 97, wherein the cancer is a B-ALL (e.g., a JAK2 (e.g., JAK2^{R683G} or JAK2^{I682F}) mutant B-ALL, with or without high CRLF2 expression).

Embodiment 99. The method of Embodiment 92, wherein the additional therapy or therapeutic agent is a BCL-2 inhibitor.

Embodiment 100. The method of Embodiment 99, wherein the cancer is selected from the group consisting of DLBCL, FL, MCL, and ALL (e.g., B-ALL).

Embodiment 101. The method of Embodiment 99, wherein the cancer is a DLBCL.

Embodiment 102. The method of Embodiment 99, wherein the cancer is a FL.

Embodiment 103. The method of Embodiment 99, wherein the cancer is an MCL.

Embodiment 104. The method of Embodiment 99, wherein the cancer is a B-ALL (e.g., an MLL-rearranged (e.g., an MLL-Af4 fusion, an MLL-Af6 fusion, an MLL-Af9 fusion, an MLL-ENL fusion, or an MLL-PTD fusion) B-ALL, or a BCL2 amplified B-ALL).

Embodiment 105. The method of Embodiment 92, wherein the additional therapy or therapeutic agent is an inhibitor of the PRC2.

Embodiment 106. The method of Embodiment 105, wherein the cancer is selected from the group consisting of DLBCL, FL, MCL, and ALL (e.g., B-ALL).

Embodiment 107. The method of Embodiment 105, wherein the cancer is a DLBCL.

Embodiment 108. The method of Embodiment 105, wherein the cancer is a FL.

Embodiment 109. The method of Embodiment 105, wherein the cancer is an MCL.

Embodiment 110. The method of Embodiment 105, wherein the cancer is a B-ALL (e.g., an MLL-rearranged (e.g., an MLL-Af4 fusion, an MLL-Af6 fusion, an MLL-Af9 fusion, an MLL-ENL fusion, or an MLL-PTD fusion) B-ALL, or a BCL2 amplified B-ALL).

Embodiment 111. The method of Embodiment 92, wherein the additional therapy or therapeutic agent is anti-CD20 therapy.

Embodiment 112. The method of Embodiment 111, wherein the cancer is DLBCL or FL.

Embodiment 113. The method of Embodiment 111, wherein the cancer is a DLBCL.

Embodiment 114. The method of Embodiment 111, wherein the cancer is a FL.

Embodiment 115. The method of Embodiment 92, wherein the additional therapy or therapeutic agent is chemotherapy.

Embodiment 116. The method of Embodiment 115, wherein the cancer is B-ALL (e.g., an MLL-rearranged (e.g., an MLL-Af4 fusion, an MLL-Af6 fusion, an MLL-Af9 fusion, an MLL-ENL fusion, or an MLL-PTD fusion) B-ALL, a Philadelphia chromosome positive B-ALL, a Philadelphia chromosome-like B-ALL, a pre-BCR+B-ALL, a B-ALL dependent on Ras signaling).

Embodiment 117. A method for treating an autoimmune condition in a subject in need thereof, the method comprising administering to the subject a therapeutically effective amount of a compound of any one of Embodiments 1-76, or a pharmaceutically acceptable salt thereof, or a pharmaceutical composition according to Embodiment 77.

Embodiment 118. A method for treating a lymphoproliferative disorder in a subject in need thereof, the method comprising administering to the subject a therapeutically effective amount of a compound of any one of Embodiments 1-76, or a pharmaceutically acceptable salt thereof, or a pharmaceutical composition according to Embodiment 77.

Embodiment 119. A method for inducing degradation of a BCL6 protein in a mammalian cell, the method comprising contacting the mammalian cell with an effective amount of a compound of any one of Embodiments 1-76, or a pharmaceutically acceptable salt thereof.

Embodiment 120. The method of Embodiment 119, wherein the contacting occurs in vivo.

Embodiment 121. The method of Embodiment 119, wherein the contacting occurs in vitro.

Embodiment 122. The method of any one of Embodiments 119-121, wherein the mammalian cell is a mammalian cancer cell.

### Exemplary Formula (I-a) Embodiments

Embodiment 1. A compound of Formula (I-a):

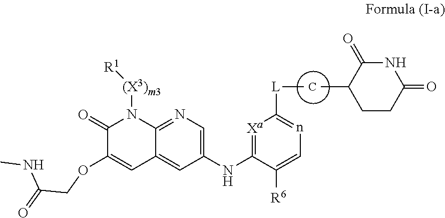

or a pharmaceutically acceptable salt thereof, wherein:
X^{a} is N or CH;
R⁶ is —F or —C₁;
m3 is 1, X³ is C₁₋₃ alkylene, and R¹ is H;
Ring C is selected from the group consisting of:

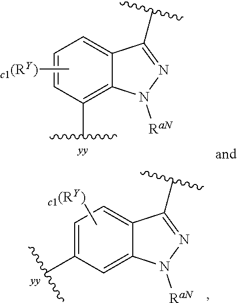

wherein: c1 is 0 or 1, RY is selected from the group consisting of halo (e.g., —F) and C₁₋₃ alkyl optionally substituted with 1-3 F, and R^{aN} is C₁₋₃ alkyl;
L is selected from the group consisting of:

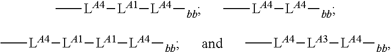

wherein bb represents the point of attachment to Ring C; and
L^{A1} is CH₂, CHMe, or CMe₂;
each L^{A4} is independently 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

Embodiment 2. The compound of Embodiment 1, wherein each L^{A4} is independently monocyclic 4-6 membered nitrogen-containing heterocyclylene optionally substituted with 1-3 R^{a}, wherein each L^{A4} contains 1-2 ring nitrogen atoms and no additional ring heteroatoms.

Embodiment 3. The compound of Embodiment 1, wherein one L^{A4} is a monocyclic 4-6 membered nitrogen-containing heterocyclylene optionally substituted with 1-3 R^{a}; and
the other L^{A4} is a bicyclic spirocyclic 6-12 (e.g., 6-10) membered nitrogen-containing heterocyclylene optionally substituted with 1-3 R^{a},
wherein each L^{A4} contains 1-2 ring nitrogen atoms and no additional ring heteroatoms.

Embodiment 4. The compound of Embodiment 1, wherein the compound is a compound of Formula (I-a-1):

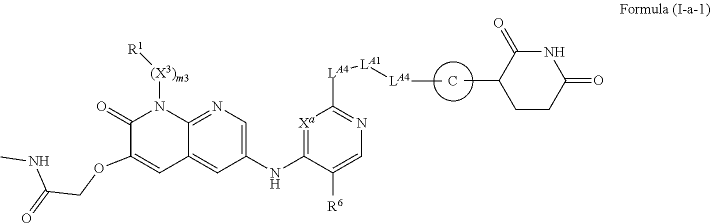

or a pharmaceutically acceptable salt thereof, wherein:
X^{a} is N or CH;
R⁶ is —F or —C₁;
m3 is 1, X³ is C₁₋₃ alkylene, and R¹ is H;
Ring C is selected from the group consisting of:

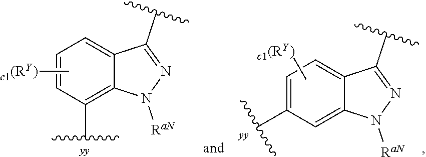

wherein: c1 is 0 or 1, RY is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂; and
each L^{A4} is independently monocyclic 4-6 membered nitrogen-containing heterocyclylene optionally substituted with 1-3 R^{a}, wherein:
each L^{A4} contains 1-2 ring nitrogen atoms and no additional ring heteroatoms, and
each R^{a} present on L^{A4} is independently selected from the group consisting of: —F, CN, C₁₋₃ alkoxy, OH, and C₁₋₃ alkyl optionally substituted with 1-3 F.

Embodiment 5. The compound of any one of Embodiments 1 or 4, wherein the compound is a compound of Formula (I-a-2):

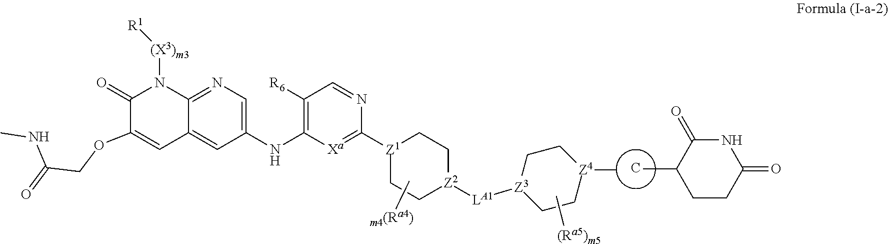

or a pharmaceutically acceptable salt thereof, wherein:
X^{a} is N or CH;
R⁶ is —F or —C₁;
m3 is 1, X³ is C₁₋₃ alkylene, and R¹ is H;
Ring C is selected from the group consisting of:

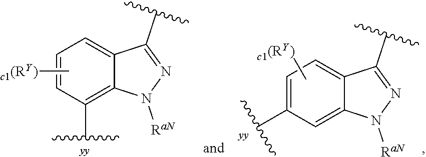

wherein: c1 is 0 or 1, RY is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂;
Z¹ and Z² are independently selected from the group consisting of: CH, CR^{a4}, and N;
Z³ and Z⁴ are independently selected from the group consisting of: CH, CRa5, and N,
provided that at least one of Z¹ and Z² is N; at least one of Z³ and Z⁴ is N; and when Z² is N, then Z³ is CH or CR^{a5};
m4 and m5 are independently selected from the group consisting of: 0, 1, and 2; and
each R^{a4} and R^{a5} is independently selected from the group consisting of: —F, CN, C₁₋₃ alkoxy, OH, and C₁₋₃ alkyl optionally substituted with 1-3 F.

Embodiment 6. The compound of Embodiment 5, wherein Z¹ is N; Z² is CH or CR^{a4}; and Z³ is N.

Embodiment 7. The compound of Embodiment 1, wherein the compound is a compound of Formula (I-a-3):

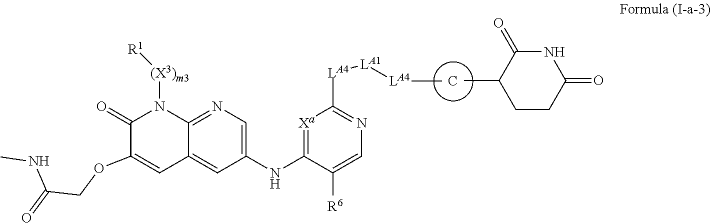

or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of:

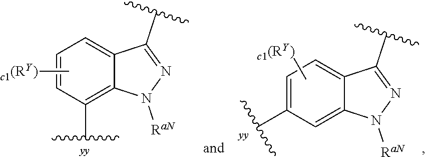

wherein: cl is 0 or 1, R^{Y} is selected from the group consisting of halo (e.g., —F) and C₁₋₃ alkyl optionally substituted with 1-3 F, and R^{aN} is C₁₋₃ alkyl;
L^{A1} is CH₂, CHMe, or C(Me)₂; and
one L^{A4} is a monocyclic 4-6 membered nitrogen-containing heterocyclylene optionally substituted with 1-3 R^{a}; and
the other L^{A4} is a bicyclic 6-12 membered nitrogen-containing heterocyclylene optionally substituted with 1-3 R^{a}, wherein:
each L^{A4} contains 1-2 ring nitrogen atoms and no additional ring heteroatom, and
each R^{a} present on L^{A4} is independently selected from the group consisting of: —F, CN, C₁₋₃ alkoxy, OH, and C₁₋₃ alkyl optionally substituted with 1-3 F.

Embodiment 8. The compound of Embodiment 7, wherein one L^{A4} is a monocyclic 4-6 membered nitrogen-containing heterocyclylene optionally substituted with 1-3 R^{a}; and
the other L^{A4} is a bicyclic spirocyclic 6-12 (e.g., 7, 9, or 11) membered nitrogen-containing heterocyclylene optionally substituted with 1-3 R^{a}.

Embodiment 9. The compound of Embodiment 7, wherein one L^{A4} is a monocyclic 4-6 membered nitrogen-containing heterocyclylene optionally substituted with 1-3 R^{a}; and
the other L^{A4} is a bicyclic bridged 6-12 (e.g., 7, 8, or 9) membered nitrogen-containing heterocyclylene optionally substituted with 1-3 R^{a}.

Embodiment 10. The compound of Embodiment 7, wherein one L^{A4} is a monocyclic 4-6 membered nitrogen-containing heterocyclylene optionally substituted with 1-3 R^{a}; and
the other L^{A4} is a bicyclic fused 6-12 (e.g., 6) membered nitrogen-containing heterocyclylene optionally substituted with 1-3 R^{a}.

Embodiment 11. The compound of any one of Embodiments 1 or 7, wherein the compound is a compound of Formula (I-a-4):

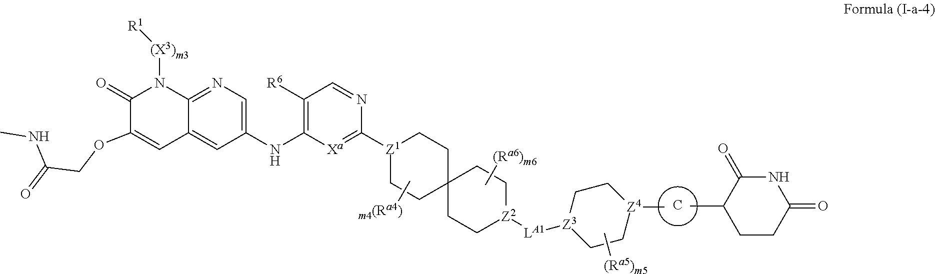

or pharmaceutically acceptable salts thereof, wherein:
X^{a} is N or CH;
R⁶ is —F or —C₁;
m3 is 1, X³ is C₁₋₃ alkylene, and R¹ is H;
Ring C is selected from the group consisting of:

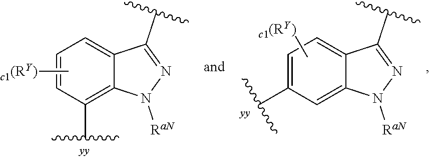

wherein: cl is 0 or 1, RY is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂;
Z¹ and Z² are independently selected from the group consisting of: CH, CR^{a4}, and N;
Z³ and Z⁴ are independently selected from the group consisting of: CH, CR^{a5}, and N,
provided that at least one of Z¹ and Z² is N; at least one of Z³ and Z⁴ is N; and when Z² is N, then Z³ is CH or CR^{a5},
m4 and m6 are independently 0 or 1;
m5 is 0, 1, or 2; and
each R^{a4}, R^{a5}, and R^{a6} is independently selected from the group consisting of: —F, CN, C₁₋₃ alkoxy, OH, and C₁₋₃ alkyl optionally substituted with 1-3 F.

Embodiment 12. The compound of Embodiment 1, wherein L is selected from the group consisting of the moieties delineated in Table L-I-a:

TABLE L-I-a

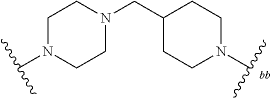

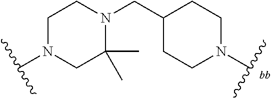

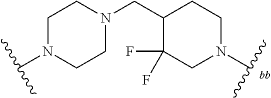

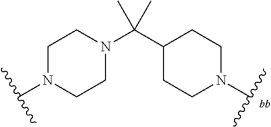

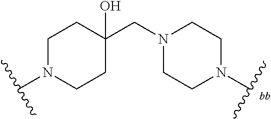

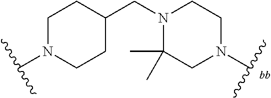

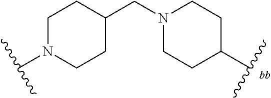

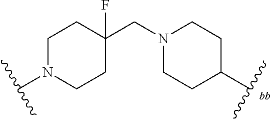

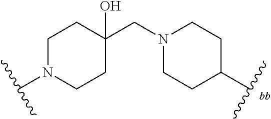

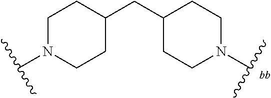

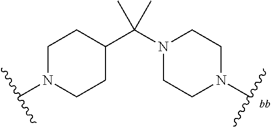

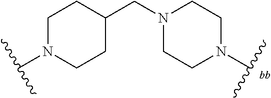

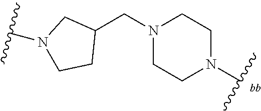

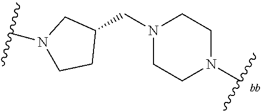

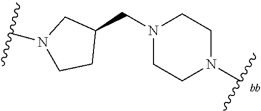

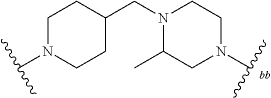

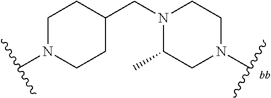

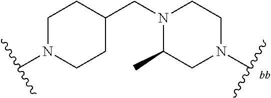

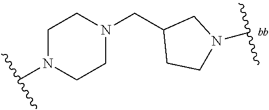

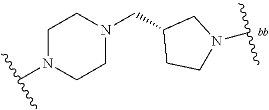

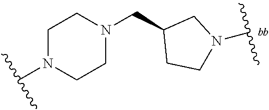

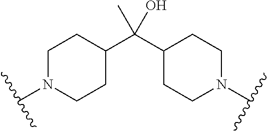

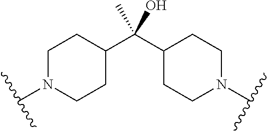

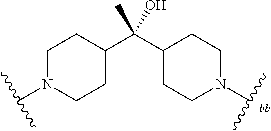

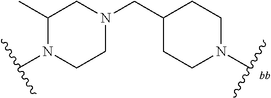

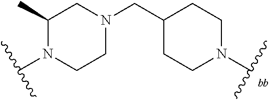

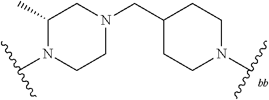

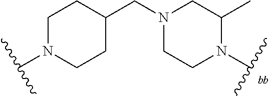

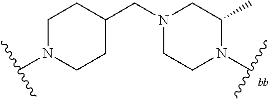

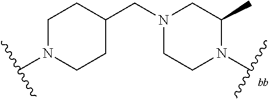

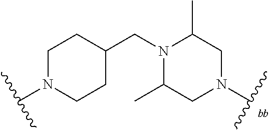

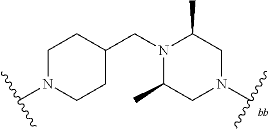

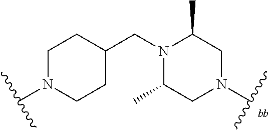

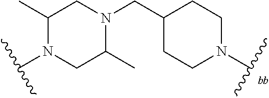

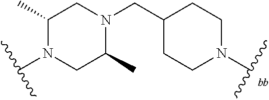

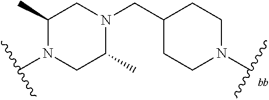

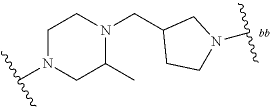

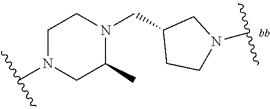

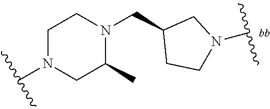

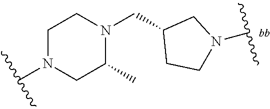

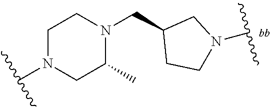

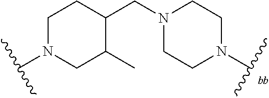

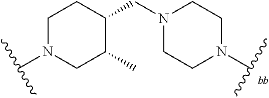

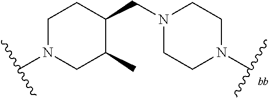

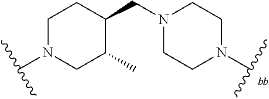

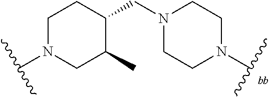

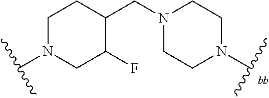

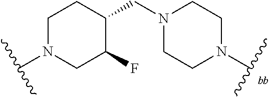

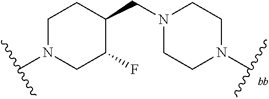

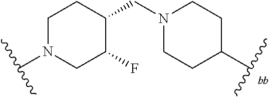

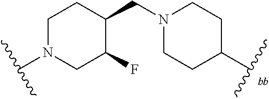

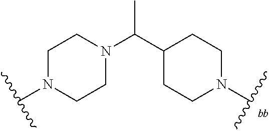

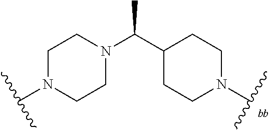

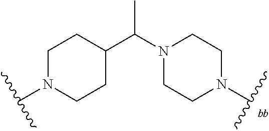

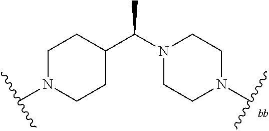

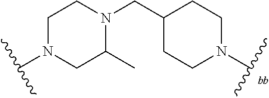

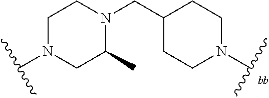

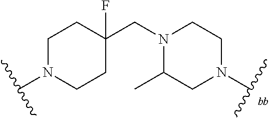

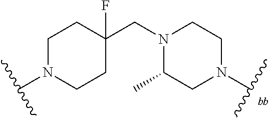

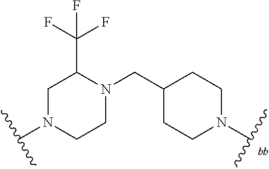

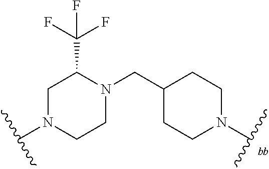

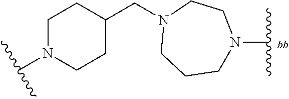

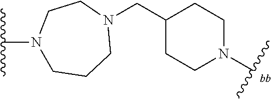

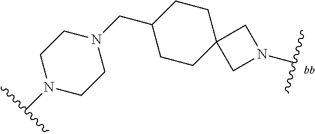

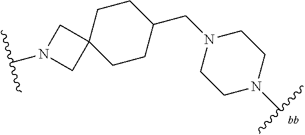

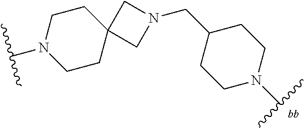

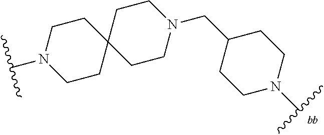

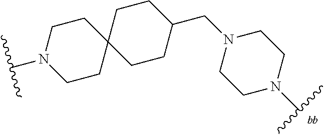

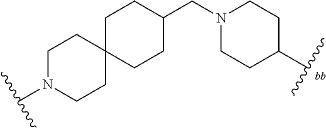

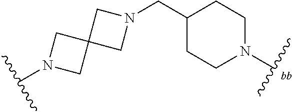

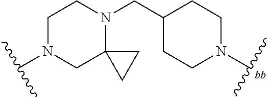

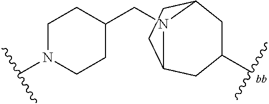

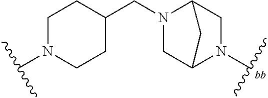

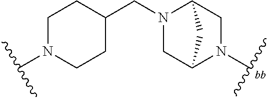

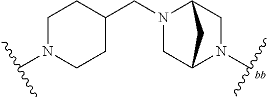

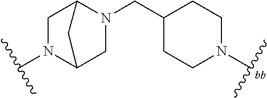

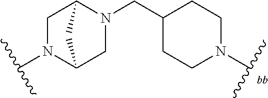

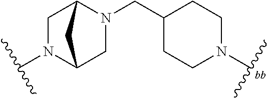

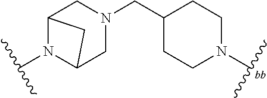

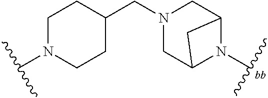

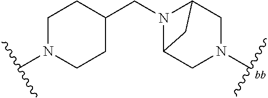

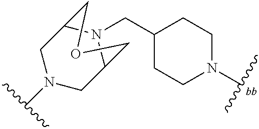

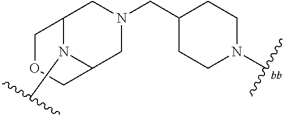

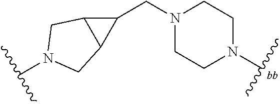

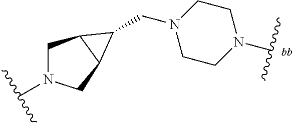

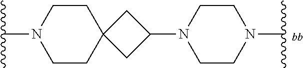

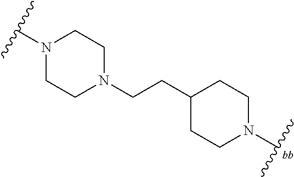

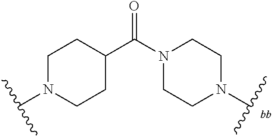

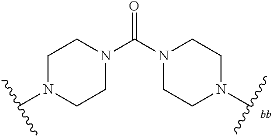

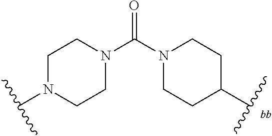

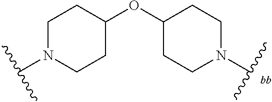

wherein bb represents the point of attachment to Ring C; or

TABLE L1a
Table L1a

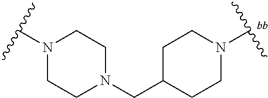

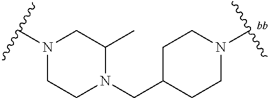

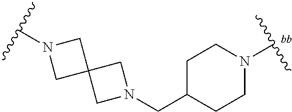

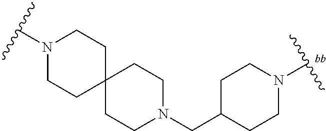

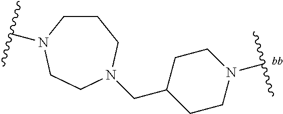

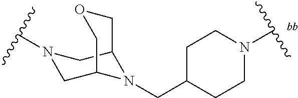

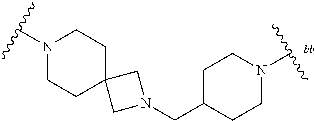

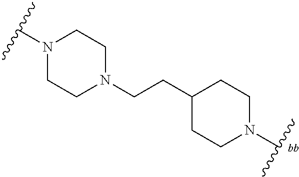

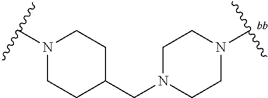

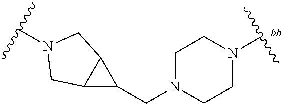

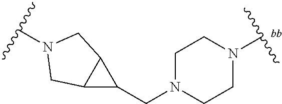

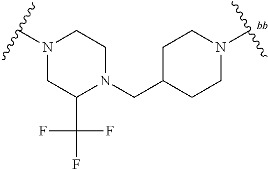

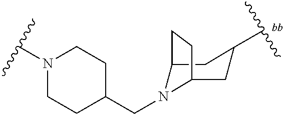

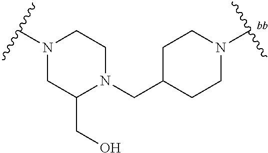

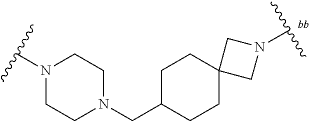

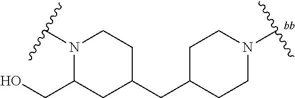

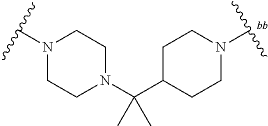

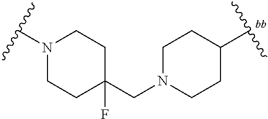

wherein bb represents the point of attachment to Ring C; or

TABLE L1a
Table L1a

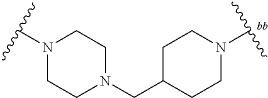

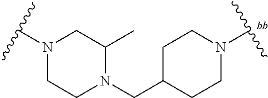

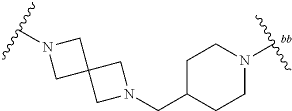

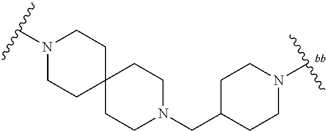

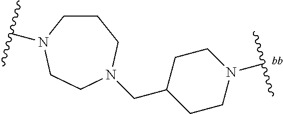

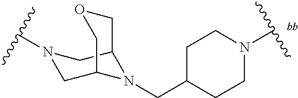

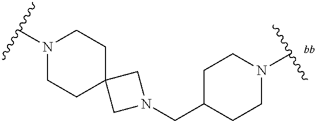

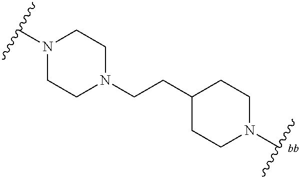

wherein bb represents the point of attachment to Ring C.

Embodiment 13. The compound of Embodiment 1, wherein the compound is selected from the group consisting of Compounds Nos. 101, 103, 104, 105, 105a, 106, 106a, 107, 110, 111, 112, 113, 114, 115, 116, 117, 118, 119, 120, 121, 124, 125, 126, 126a, 127, 127a, 128, 129, 130, 130a, 131, 131a, 132, 133, 134, 135, 136, 136a, 139, 140, 141, 141a, 143, 145, 149, 152, 158, 160, 161, 162, 163, 163a, 167, 168, 168a, 168b, 169, 169a, 169b, 171, 172, 173, 174, 174a, 174b, 175, 175a, 175b, 176, 176a, 176b, 176c, 176d, 177, 177a, 177b, 177c, 177d, 178, 179, 179a, 179b, 187, 188, 188a, 188b, 189, 189a, 190, 191, 192, 192a, 192b, 193, 193a, 195, 195a, 196, 197, 197a, 197b, 200, 203, 204, 205, 209, 210, 210a, 210b, 211, 212, 214, 215, 215a, 215b, 216, 216a, 216b, 216c, 216d, 217, 217a, 217b, 218, 218a, 218b, 219, 220, 223, 224, 225, 228, 228a, 228b, 229, 230, 235, 236, 237, 238, 238a, 238b, 239, 240, 241, 242, 242a, and 242b as depicted in Table C₁, or a pharmaceutically acceptable salt thereof; or

Compounds Nos. 101, 103, 104, 105, 105a, 106, 106a, 107, 110, 111, 112, 113, 114, 115, 116, 117, 118, 119, 120, 121, 124, 125, 126, 126a, 127, 127a, 128, 129, 130, 130a, 131, 131a, 132, 133, 134, 135, 136, 136a, 139, 140, 141, 141a, 143, 145, 149, 152, 158, 160, 161, 162, 163, 163a, 167, 168, 168a, 168b, 169, 169a, 169b, 171, 172, 173, 174, 174a, 174b, 175, 175a, 175b, 176, 176a, 176b, 176c, 176d, 177, 177a, 177b, 177c, 177d, 178, 179, 179a, 179b, 187, 188, 188a, 188b, 189, 189a, 190, 191, 192, 192a, 192b, 193, 193a, 195, 195a, 196, 197, 197a, 197b, 200, 203, 204, 205, 209, 210, 210a, 210b, 211, 212, 214, 215, 215a, 215b, 216, 216a, 216b, 216c, 216d, 217, 217a, 217b, 218, 218a, 218b, 219, 220, 223, 224, 225, 228, 228a, 228b, 229, and 230 as depicted in Table C1, or a pharmaceutically acceptable salt thereof; or

Compounds Nos. 101, 103, 104, 105, 105a, 106, 106a, 107, 110, 111, 112, 113, 114, 115, 116, 117, 118, 119, 120, 121, 124, 125, 126, 126a, 127, 127a, 128, 129, 130, 130a, 131, 131a, 132, 133, 134, 135, 136, 136a, 139, 140, 141, 141a, 143, 145, 149, 152, 158, 160, and 161 as depicted in Table C1, or a pharmaceutically acceptable salt thereof, or

Compounds Nos. 101, 103, 104, 105, 105a, 106, 106a, 107, 110, 111, 112, 113, 114, 115, 116, 117, 118, 119, 120, 121, 124, 125, 126, 126a, 127, 127a, 128, 129, 130, 130a, 131, and 131a as depicted in Table C1, or a pharmaceutically acceptable salt thereof.

Embodiment 14. A pharmaceutical composition comprising a compound of any one of Embodiments 1-13, or a pharmaceutically acceptable salt thereof, and a pharmaceutically

Embodiment 15. A compound of any one of Embodiments 1-13, or a pharmaceutically acceptable salt thereof, or the pharmaceutical composition of Embodiment 14 for use in treatment.

Embodiment 16. A compound of any one of Embodiments 1-13 or a pharmaceutically acceptable salt thereof, or the pharmaceutical composition of Embodiment 14 for use in the treatment of a cancer selected from the group consisting of a hematological cancer, breast cancer, gastrointestinal cancer, brain cancer, and lung cancer.

Embodiment 17. The compound or pharmaceutical composition of Embodiment 16, wherein the hematological cancer is selected from the group consisting of diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), peripheral T-cell lymphoma (PTCL), chronic lymphocytic leukemia (CLL), acute lymphocytic leukemia (ALL), and chronic myeloid leukemia (CML); optionally wherein the hematological cancer is selected from the group consisting of diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), chronic lymphocytic leukemia (CLL), acute lymphocytic leukemia (ALL), and chronic myeloid leukemia (CML); optionally wherein the hematological cancer is selected from the group consisting of DLBCL, FL, MCL, BL, PTCL, and ALL (e.g., B-ALL); optionally wherein the hematological cancer is FL or DLBCL; optionally wherein in the hematological cancer is DLBCL.

Embodiment 18. The compound or pharmaceutical composition of Embodiment 15 and an additional therapeutic agent or therapy selected from the group consisting of a PI3K inhibitor, an Abl inhibitor (e.g., a BCR-Abl inhibitor), a BTK inhibitor, a JAK inhibitor, a BRaf inhibitor, a MEK inhibitor, a BCL-2 inhibitor, a Bcl-XL inhibitor, an XPO1 inhibitor, an inhibitor of the polycomb repressive complex 2 (PRC2), an immunomodulatory imide drug, anti-CD19 therapy, anti-CD20 therapy, anti-CD3 therapy, chemotherapy, and a combination thereof for use in treatment; or
an additional therapeutic agent or therapy selected from the group consisting of a PI3K inhibitor, a BTK inhibitor, a JAK inhibitor, a BRaf inhibitor, a MEK inhibitor, a BCL-2 inhibitor, a BCL-XL inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof for use in treatment; or
an additional therapeutic agent or therapy selected from the group consisting of a PI3K inhibitor, a BTK inhibitor, a JAK inhibitor, a BRaf inhibitor, a MEK inhibitor, a Bcl-2 inhibitor, a Bcl-xL inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof Embodiment 1. for use in treatment; or
an additional therapeutic agent or therapy selected from the group consisting of a PI3K inhibitor, a BTK inhibitor, a JAK inhibitor, a BRaf inhibitor, a MEK inhibitor, a Bcl-2 inhibitor, a Bcl-xL inhibitor, an XPO1 inhibitor, an immunomodulatory imide drug, anti-CD19 therapy, anti-CD20 therapy, anti-CD3 therapy, and a combination thereof for use in treatment.

Embodiment 19. The compound or pharmaceutical composition of Embodiment 16 and an additional therapeutic agent or therapy selected from the group consisting of a PI3K inhibitor, an Abl inhibitor (e.g., a BCR-Abl inhibitor), a BTK inhibitor, a JAK inhibitor, a BRaf inhibitor, a MEK inhibitor, a BCL-2 inhibitor, a Bcl-XL inhibitor, an XPO1 inhibitor, an inhibitor of the polycomb repressive complex 2 (PRC2), an immunomodulatory imide drug, anti-CD19 therapy, anti-CD20 therapy, anti-CD3 therapy, chemotherapy, and a combination thereof; or
an additional therapeutic agent or therapy selected from the group consisting of a PI3K inhibitor, a BTK inhibitor, a JAK inhibitor, a BRaf inhibitor, a MEK inhibitor, a BCL-2 inhibitor, a BCL-XL inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof; or
an additional therapeutic agent or therapy selected from the group consisting of a PI3K inhibitor, a BTK inhibitor, a JAK inhibitor, a BRaf inhibitor, a MEK inhibitor, a Bcl-2 inhibitor, a Bcl-xL inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof, or
an additional therapeutic agent or therapy selected from the group consisting of a PI3K inhibitor, a BTK inhibitor, a JAK inhibitor, a BRaf inhibitor, a MEK inhibitor, a Bcl-2 inhibitor, a Bcl-xL inhibitor, an XPO1 inhibitor, an immunomodulatory imide drug, anti-CD19 therapy, anti-CD20 therapy, anti-CD3 therapy, and a combination thereof.

Embodiment 20. A compound of any one of Embodiments 1-13, or a pharmaceutically acceptable salt thereof, or the pharmaceutical composition of Embodiment 14 for use in a method of treating cancer selected from the group consisting of diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), peripheral T-cell lymphoma (PTCL), chronic lymphocytic leukemia (CLL), acute lymphocytic leukemia (ALL), and chronic myeloid leukemia (CML); optionally wherein the cancer is selected from the group consisting of diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), chronic lymphocytic leukemia (CLL), acute lymphocytic leukemia (ALL), and chronic myeloid leukemia (CML); optionally wherein the cancer is selected from the group consisting of DLBCL, FL, MCL, BL, PTCL, and ALL (e.g., B-ALL); optionally wherein the cancer is FL or DLBCL; optionally wherein in the cancer is DLBCL, the method comprising administering to a subject a therapeutically effective amount of a compound of Embodiments 1-13, or a pharmaceutically acceptable salt thereof, or the pharmaceutical composition of Embodiment 14.

Embodiment 21. The compound or pharmaceutical composition of Embodiment 20, wherein the method comprises administering to the subject a therapeutically effective amount of an additional therapeutic agent or therapy selected from the group consisting of a PI3K inhibitor, an Abl inhibitor (e.g., a BCR-Abl inhibitor), a BTK inhibitor, a JAK inhibitor, a BRaf inhibitor, a MEK inhibitor, a BCL-2 inhibitor, a Bcl-XL inhibitor, an XPO1 inhibitor, an inhibitor of the polycomb repressive complex 2 (PRC2), an immunomodulatory imide drug, anti-CD19 therapy, anti-CD20 therapy, anti-CD3 therapy, chemotherapy, and a combination thereof; or
an additional therapeutic agent or therapy selected from the group consisting of a PI3K inhibitor, a BTK inhibitor, a JAK inhibitor, a BRaf inhibitor, a MEK inhibitor, a BCL-2 inhibitor, a BCL-XL inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof; or
an additional therapeutic agent or therapy selected from the group consisting of a PI3K inhibitor, a BTK inhibitor, a JAK inhibitor, a BRaf inhibitor, a MEK inhibitor, a Bcl-2 inhibitor, a Bcl-xL inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof; or
an additional therapeutic agent or therapy selected from the group consisting of a PI3K inhibitor, a BTK inhibitor, a JAK inhibitor, a BRaf inhibitor, a MEK inhibitor, a Bcl-2 inhibitor, a Bcl-xL inhibitor, an XPO1 inhibitor, an immunomodulatory imide drug, anti-CD19 therapy, anti-CD20 therapy, anti-CD3 therapy, and a combination thereof.

Embodiment 22. The compound or pharmaceutical composition of Embodiment 46, wherein the compound of any one of Embodiments 1-13, or a pharmaceutically acceptable salt thereof, or the pharmaceutical composition of Embodiment 14, and the additional therapeutic agent or therapy are administered simultaneously, separately, or sequentially.

### Exemplary Formula (I) Embodiments

Embodiment 1. A compound of Formula (I):

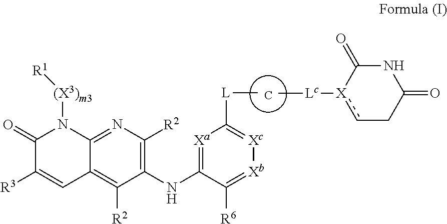

or a pharmaceutically acceptable salt thereof, wherein:
R³ is

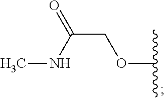 [SMILES: *OCC(=O)NC]

X^{a} is N; X^{c} is N; and X^{b} is CR^{x1} (e.g., CH); or X^{a} is CH; X^{c} is N; and X^{b} is CR^{x1} (e.g., CH);
R⁶ is —F or —C₁;
m3 is 1, X³ is C₁₋₃ alkylene, and R¹ is H;
Ring C is

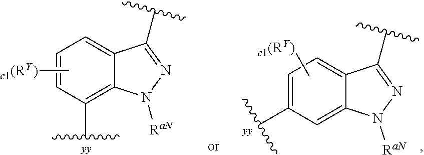

wherein: c1 is 0; or cl is 1 and R^{Y} is halo (e.g., —F);
R^{aN} is C₁₋₃ alkyl (e.g., methyl);
X is CH;
L^{C} is a bond;
L is selected from the group consisting of:

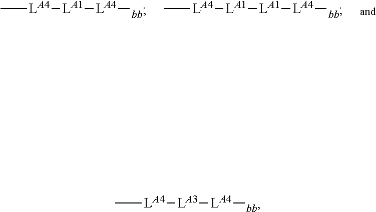

wherein bb represents the point of attachment to Ring C; and

L^{A1} is CH₂;
each L^{A4} is independently a 4-10 membered heterocyclylene optionally substituted with 1-3 R^{a}, wherein:
each R^{a} present on L^{A4} is independently selected from the group consisting of: F, methyl, CF₃, CHF₂, and CH₂F.

Embodiment 2. The compound of Embodiment 1, wherein one L^{A4} is a monocyclic 4-6 membered monocyclic nitrogen-containing heterocyclylene optionally substituted with 1-3 R^{a}; and the other L^{A4} is a bicyclic 6-12 membered nitrogen-containing heterocyclylene optionally substituted with 1-3 Rav

Embodiment 3. The compound of Embodiment 1, wherein one L^{A4} is a monocyclic 4-6 membered monocyclic nitrogen-containing heterocyclylene optionally substituted with 1-3 R^{a}; and the other L^{A4} is a bicyclic 6-12 membered nitrogen-containing heterocyclylene optionally substituted with 1-3 R^{a}

Embodiment 4. The compound of Embodiment 1, wherein L is -L^{A4}-L^{A1}-L^{A4}-_{bb}, wherein bb represents the point of attachment to Ring C.

Embodiment 5. The compound of Embodiment 4, wherein one L^{A4} is a monocyclic 4-6 membered nitrogen-containing heterocyclylene optionally substituted with 1-3 R^{a}; and
the other L^{A4} is a bicyclic spirocyclic 6-10 membered nitrogen-containing heterocyclylene optionally substituted with 1-3 R^{a}.

Embodiment 6. The compound of Embodiment 4, wherein one L^{A4} is a monocyclic 4-6 membered nitrogen-containing heterocyclylene optionally substituted with 1-3 R^{a}; and
the other L^{A4} is a bicyclic bridged 6-10 membered nitrogen-containing heterocyclylene optionally substituted with 1-3 R^{a}.

Embodiment 7. The compound of Embodiment 1, wherein L is selected from the group consisting of the moieties delineated in Table L-I-a:

TABLE L1a

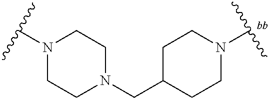

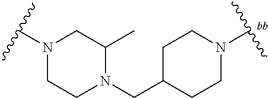

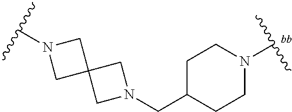

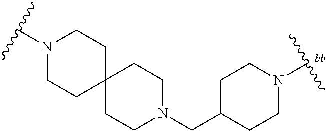

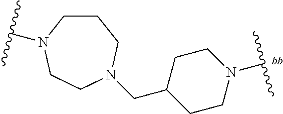

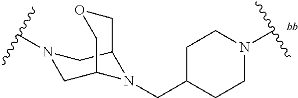

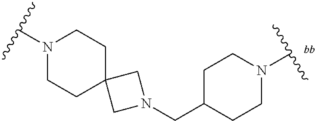

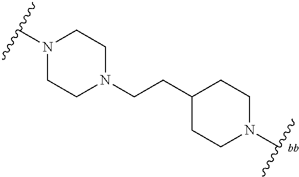

wherein bb represents the point of attachment to Ring C.

Embodiment 8. The compound of Embodiment 1, wherein the compound is selected from the group consisting of Compounds Nos. 101, 103, 104, 105, 105a, 106, 106a, 107, 110, 111, 112, 113, 114, 115, 116, 117, 118, 119, 120, 121, 124, 125, 126, 126a, 127, 127a, 128, 129, 130, 130a, 131, and 131a as depicted in Table C1, or a pharmaceutically acceptable salt thereof.

Embodiment 9. A pharmaceutical composition comprising a compound of any one of Embodiments 1-8, or a pharmaceutically acceptable salt thereof, and a pharmaceutically acceptable excipient.

Embodiment 10. A compound of any one of Embodiments 1-8, or a pharmaceutically acceptable salt thereof, or the pharmaceutical composition of Embodiment 9 for use in treatment.

Embodiment 11. A compound of any one of Embodiments 1-8 or a pharmaceutically acceptable salt thereof, or the pharmaceutical composition of Embodiment 9 for use in the treatment of a cancer selected from the group consisting of a hematological cancer, breast cancer, gastrointestinal cancer, brain cancer, and lung cancer.

Embodiment 12. The compound or pharmaceutical composition of Embodiment 11, wherein the hematological cancer is selected from the group consisting of diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), chronic lymphocytic leukemia (CLL), acute lymphocytic leukemia (ALL), and chronic myeloid leukemia (CML); optionally wherein in the hematological cancer is DLBCL.

Embodiment 13. The compound or pharmaceutical composition of Embodiment 11 and an additional therapeutic agent or therapy selected from the group consisting of a PI3K inhibitor, a BTK inhibitor, a JAK inhibitor, a BRaf inhibitor, a MEK inhibitor, a Bcl-2 inhibitor, a Bcl-xL inhibitor, an XPO1 inhibitor, an immunomodulatory imide drug, anti-CD19 therapy, anti-CD20 therapy, anti-CD3 therapy, and a combination thereof for use in treatment.

Embodiment 14. The compound or pharmaceutical composition of Embodiment 12 and an additional therapeutic agent or therapy selected from the group consisting of a PI3K inhibitor, a BTK inhibitor, a JAK inhibitor, a BRaf inhibitor, a MEK inhibitor, a Bcl-2 inhibitor, a Bcl-xL inhibitor, an XPO1 inhibitor, an immunomodulatory imide drug, anti-CD19 therapy, anti-CD20 therapy, anti-CD3 therapy, and a combination thereof.

Embodiment 15. A compound of any one of Embodiments 1-8, or a pharmaceutically acceptable salt thereof, or the pharmaceutical composition of Embodiment 9 for use in a method of treating cancer selected from the group consisting of diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), chronic lymphocytic leukemia (CLL), acute lymphocytic leukemia (ALL), and chronic myeloid leukemia (CML); optionally wherein in the cancer is DLBCL, the method comprising administering to a subject a therapeutically effective amount of a compound of Embodiments 1-8, or a pharmaceutically acceptable salt thereof, or the pharmaceutical composition of Embodiment 9.

Embodiment 16. The compound or pharmaceutical composition of Embodiment 15, wherein the method comprises administering to the subject a therapeutically effective amount of an additional therapeutic agent or therapy selected from the group consisting of a PI3K inhibitor, a BTK inhibitor, a JAK inhibitor, a BRaf inhibitor, a MEK inhibitor, a Bcl-2 inhibitor, a Bcl-xL inhibitor, an XPO1 inhibitor, an immunomodulatory imide drug, anti-CD19 therapy, anti-CD20 therapy, anti-CD3 therapy, and a combination thereof.

Embodiment 17. The compound or pharmaceutical composition of Embodiment 16, wherein the compound of any one of Embodiments 1-8, or a pharmaceutically acceptable salt thereof, or the pharmaceutical composition of Embodiment 9, and the additional therapeutic agent or therapy are administered simultaneously, separately, or sequentially.

### Exemplary Formula (I) Embodiments

Embodiment 1. A compound of Formula (I):

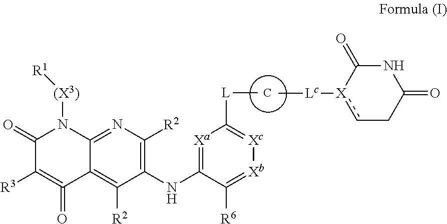

or a pharmaceutically acceptable salt thereof, wherein:
R¹ is selected from the group consisting of: H, halo, cyano, and R^{b1};
m3 is 0, 1, 2, or 3;
each X³ is independently selected from the group consisting of: —O—, —NR^{f}—, —C(═O)—, and C₁₋₃ alkylene optionally substituted with 1-3 R^{c};
provided that the N—(X³) m3-R¹ moiety does not contain any O—O, N—O, N—N, O-halo, or N-halo bonds;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e};
R³ is selected from the group consisting of:
-A¹-C(R⁴R⁴)-A² and —CH═CH-A², wherein:
A¹ is —O— or —S—;
each R⁴ is independently selected from the group consisting of: H, C₁₋₆ alkyl, and C₁₋₆ haloalkyl; or
the pair of R⁴ taken together with the carbon atom to which each is attached form a C₃-6 cycloalkyl ring or a 4-8 membered heterocyclyl ring, wherein the C₃₋₆ cycloalkyl ring or 4-8 membered heterocyclyl ring is optionally substituted with 1-3 R⁸;
A² is selected from the group consisting of: —C(O)OH, —C(O)NH₂, —C(O)R^{3A}, —C(O)OR^{3A}, —C(O)NR^{3A}R^{f}, —S(O)₁₋₂ (C₁₋₆ alkyl), —P(O)—(C₁₋₆ alkyl) 2, and —C(═NH) NH₂, wherein:
R^{3A} is selected from the group consisting of: C₁₋₆ alkyl, C₃₋₆ alkenyl, C₃₋₆ alkynyl, C₃₋₆ cycloalkyl, and 3-8 membered heterocyclyl, each of which is optionally substituted with 1-6 substituents independently selected from the group consisting of R^{a} and —(C₀₋₃ alkylene)-R^{b1};
X^{a} and X^{c} are independently selected from the group consisting of: N, CH, and CF, provided that one or both of X^{a} and X^{c} is N;
X^{b} is selected from the group consisting of N and CR^{x1};
R⁶ and Ral are each independently selected from the group consisting of: H, halo, C₁₋₂ alkyl, C₁₋₂ haloalkyl, C₁₋₂ alkoxy, CN, and —C═CH;
L is -(L^{A})ₙ₁-, wherein L^{A} and n1 are defined according to (AA) or (BB):
   (AA)
n1 is an integer from 1 to 15; and
each L^{A} is independently selected from the group consisting of: L^{A1}, L^{A3}, and L^{A4}, provided that 1-3 occurrences of L^{A} is L^{A4};
   (BB)
n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, O—O, N—N, N—S(O) o, or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, S(O)₀₋₂ (C₁₋₆ alkyl), S(O)₀₋₂ (C₁₋₆ haloalkyl), S(O)₁₋₂N(R^{f})₂, —R^{b}, and C₁₋₆ alkyl optionally substituted with 1-6 R^{c};
Ring C is selected from the group consisting of:

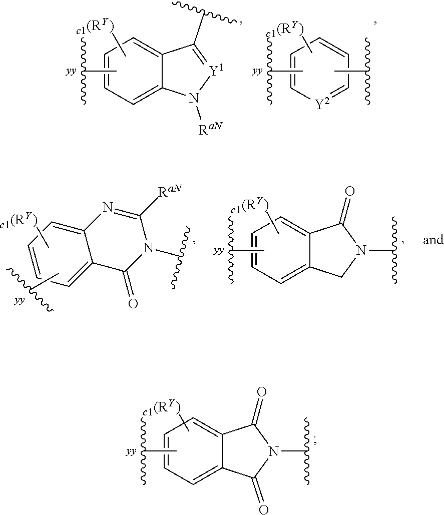

c1 is 0, 1, 2, or 3;
each R^{Y} is independently selected from the group consisting of: R^{a} and R^{b};
R^{aN} is H or C₁₋₆ alkyl optionally substituted with 1-3 R^{c};

Y¹ and Y² are independently N, CH, or CRY;

yy represents the point of attachment to L;
X is CH, C, or N;

the [symbol] is a single bond or a double bond;

L^{C} is selected from the group consisting of: a bond, —CH₂—, —CHR^{a}—, —C(R^{a})₂—, —C(═O)—, —N(R^{d})—, and O, provided that when X is N, then L^{C} is other than O; and
further provided that when Ring Cis attached to -L^{C}- via a ring nitrogen, then X is CH, and L^{C} is a bond;
each R^{a} is independently selected from the group consisting of:
(a) halo;
(b) cyano;
(c) —OH;
(d) oxo;
(e) C₁₋₆ alkoxy optionally substituted with 1-6 R^{c};
(f) —NR^{d}R^{e};
(g) C(═O) C₁₋₆ alkyl optionally substituted with 1-6 R^{c};
(h) C(═O)OH;
(i) C(═O)OC₁₋₆ alkyl;
(j) C(═O)OC₁₋₆ haloalkyl;
(k) C(═O)N(R^{f})₂;
(l) S(O)₀₋₂ (C₁₋₆ alkyl);
(m) S(O)₀₋₂ (C₁₋₆ haloalkyl);
(n) S(O)₁₋₂N(R^{f}) 2; and
(o) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl C(═O)OC₁₋₆ haloalkyl, C(═O)OH, C(═O)N(R^{f})₂, S(O)₀₋₂ (C₁₋₆ alkyl), S(O)₀₋₂ (C₁₋₆ haloalkyl), and S(O)₁₋₂N(R^{f})₂;
each R^{d} and R^{e} is independently selected from the group consisting of: H, C(═O) C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂ (C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, cyano, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H) (C₁₋₃ alkyl), and —(C₀₋₃ alkylene)-N(C₁₋₃ alkyl)₂.

Embodiment 2. The compound of Embodiment 1, wherein R³ is -A¹-C(R⁴R⁴)-A².

Embodiment 3. The compound of Embodiment 1 or 2, wherein A¹ is —O—.

Embodiment 4. The compound of any one of Embodiments 1-3, wherein each R⁴ is H.

Embodiment 5. The compound of any one of Embodiments 1-4, wherein A² is —C(O)NH₂ or —C(O)NR^{3A}R^{f}

Embodiment 6. The compound of Embodiment 5, wherein R^{3A} is C₁₋₃ alkyl optionally substituted with 1-6 R^{c}.

Embodiment 7. The compound of Embodiments 1-6, wherein A² is —C(O)NH₂, —C(O)NHMe, or —C(O)NMe₂.

Embodiment 8. The compound of any one of Embodiments 1-7, wherein A² is —C(O)NHMe.

Embodiment 9. The compound of Embodiments 1 or 2, wherein R³ is -A¹-C(R⁴R⁴)-A²; A¹ is O; each R⁴ is H; and A² is —C(O)NH₂ or —C(O)NR^{3A}R^{f}, wherein R^{3A} is C₁₋₃ alkyl optionally substituted with 1-6 R^{c}.

Embodiment 10. The compound of Embodiment 9, wherein A² is —C(O)NH₂, —C(O)NHMe, or —C(O)NMe₂.

Embodiment 11. The compound of Embodiment 1, wherein R³ is

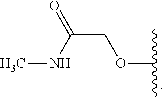 [SMILES: *OCC(=O)NC]

Embodiment 12. The compound of any one of Embodiments 1-11, wherein X^{a} is N; X^{c} is N; and X^{b} is CR^{x1} (e.g., CH).

Embodiment 13. The compound of any one of Embodiments 1-11, wherein X^{a} is CH; X^{c} is N; and X^{b} is CR^{x1} (e.g., CH).

Embodiment 14. The compound of any one of Embodiments 1-13, wherein R⁶ is —Cl or —F.

Embodiment 15. The compound of any one of Embodiments 1-14, wherein each R² is H.

Embodiment 16. The compound of any one of Embodiments 1-15, wherein m3 is 0.

Embodiment 17. The compound of any one of Embodiments 1-15, wherein m3 is 1; and X³ is C₁₋₃ alkylene (e.g., methylene, ethylene, or isopropylene).

Embodiment 18. The compound of any one of Embodiments 1-17, wherein R¹ is H.

Embodiment 19. The compound of any one of Embodiments 1-15, wherein m3 is 1; X³ is methylene, ethylene, or isopropylene; and R¹ is H.

Embodiment 20. The compound of Embodiment 1, wherein:
R³ is -A¹-C(R⁴R⁴)-A², wherein A¹ is O; each R⁴ is H; and A² is —C(O)NH₂ or —C(O)NR^{3A}R^{f}, wherein R^{3A} is C₁₋₃ alkyl optionally substituted with 1-6 R^{c};
each R² is H;
X^{a} is N or CH;
X^{c} is N;
X^{b} is CH; and
R⁶ is —F or —C₁.

Embodiment 21. The compound of Embodiment 20, wherein m3 is 1; X³ is methylene, ethylene, or isopropylene; and R¹ is H.

Embodiment 22. The compound of Embodiments 20 or 21, wherein X^{a} is N.

Embodiment 23. The compound of any one of Embodiments 1-22, wherein Ring Cis

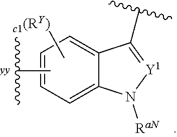

Embodiment 24. The compound of Embodiments 1-23, wherein Ring C is

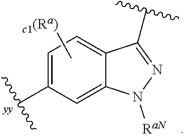

Embodiment 25. The compound of Embodiments 1-23, wherein Ring C is

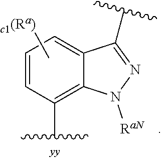

Embodiment 26. The compound of any one of Embodiments 1-25, wherein cl is 0.

Embodiment 27. The compound of any one of Embodiments 1-25, wherein cl is 1; and RY is halo (e.g., —F).

Embodiment 28. The compound of anyone of Embodiments 1-27, wherein X is CH.

Embodiment 29. The compound of any one of Embodiments 1-28, wherein R^{aN} is C₁₋₃ alkyl (e.g., methyl).

Embodiment 30. The compound of any one of Embodiments 1-29, wherein L^{C} is a bond.

Embodiment 31. The compound of any one of Embodiments 1-22, wherein the

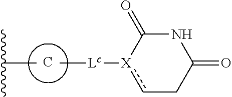

moiety is selected from the group consisting of:

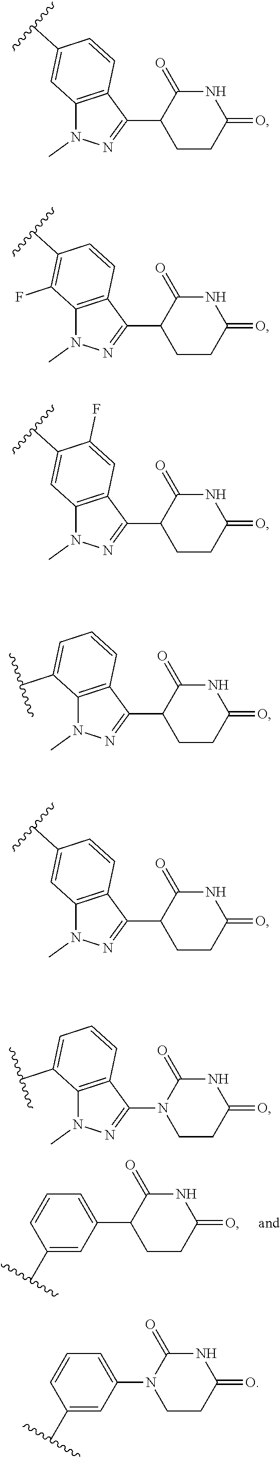

Embodiment 32. The compound of any one of Embodiments 1-22 or 31, wherein the

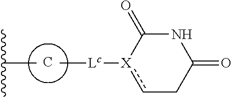

moiety is selected from the group consisting of:

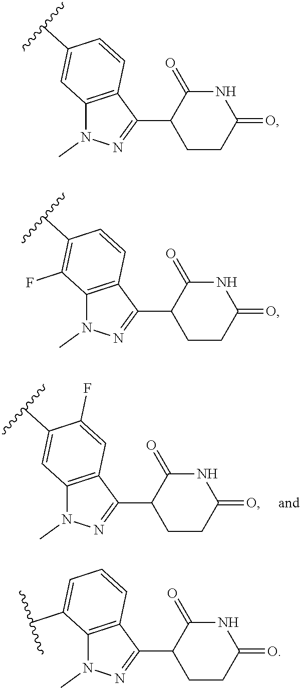

Embodiment 33. The compound of any one of Embodiments 1-32, wherein L is -(L^{A})ₙ₁—, wherein L^{A} and n1 are defined according to (AA).

Embodiment 34. The compound of any one of Embodiments 1-33, wherein n1 is an integer from 1 to 5.

Embodiment 35. The compound of any one of Embodiments 1-34, wherein n1 is an integer from 2 to 5 (e.g., 2 or 3).

Embodiment 36. The compound of Embodiment 1, wherein the compound is selected from the group consisting of Compound No. 101, 102, 103, 104, 105, 105a, 106, 106a, 107, 108, 109, 110, 111, 112, 113, 114, 115, 116, 117, 118, 119, 120, 121, 122, 122a, 122b, 123, 123a, 123b, 124, 125, 126, 126a, 127, 127a, 128, 129, 130, 130a, 131, 131a, 132, 133, 134, 135, 136, 136a, 137, 138, 139, 140, 141, 141a, 142, 143, 144, 145, 146, 146a, 146b, 147, 148, 149, 150, 151, 151a, 151b, 152, 153, 153a, 153b, 154, 155, 155a, 155b, 156, 156a, 156b, 156c, 156d, 157, 158, 159, 159a, 159b, 160, 161, 162, 163, 163a, 164, 165, 166, 167, 168, 168a, 168b, 169, 169a, 169b, 170, 171, 172, 173, 174, 174a, 174b, 175, 175a, 175b, 176, 176a, 176b, 176c, 176d, 177, 177a, 177b, 177c, 177d, 178, 179, 179a, 179b, 180, 180a, 180b, 180c, 180d, 181, 182, 183, 184, 185, 185a, 185b, 185c, 185d, 186, 187, 188, 188a, 188b, 189, 189a, 190, 191, 192, 192a, 192b, 193, 193a, 194, 194a, 194b, 195, 195a, 196, 197, 197a, 1976, 198, 199, 200, 201, 201a, 202, 203, 204, 205, 206, 207, 208, 208a, 208b, 209, 210, 210a, 210b, 211, 212, 213, 214, 215, 215a, 215b, 216, 216a, 216b, 216c, 216d, 217, 217a, 217b, 218, 218a, 218b, 219, 220, 221, 221a, 221b, 221c, 221d, 222, 223, 224, 225, 226, 226a, 227, 227a, 228, 228a, 228b, 229, 230, 231, 231a, 231b, 232, 232a, 232b, 233, 233a, 235, 236, 237, 238, 238a, 238b, 239, 240, 241, 242, 242a, and 242b as depicted in Table C1, or a pharmaceutically acceptable salt thereof; or

Compound No. 101, 102, 103, 104, 105, 105a, 106, 106a, 107, 108, 109, 110, 111, 112, 113, 114, 115, 116, 117, 118, 119, 120, 121, 122, 122a, 122b, 123, 123a, 123b, 124, 125, 126, 126a, 127, 127a, 128, 129, 130, 130a, 131, 131a, 132, 133, 134, 135, 136, 136a, 137, 138, 139, 140, 141, 141a, 142, 143, 144, 145, 146, 146a, 146b, 147, 148, 149, 150, 151, 151a, 151b, 152, 153, 153a, 153b, 154, 155, 155a, 155b, 156, 156a, 156b, 156c, 156d, 157, 158, 159, 159a, 159b, 160, 161, 162, 163, 163a, 164, 165, 166, 167, 168, 168a, 168b, 169, 169a, 169b, 170, 171, 172, 173, 174, 174a, 174b, 175, 175a, 175b, 176, 176a, 176b, 176c, 176d, 177, 177a, 1776, 177c, 177d, 178, 179, 179a, 179b, 180, 180a, 180b, 180c, 180d, 181, 182, 183, 184, 185, 185a, 185b, 185c, 185d, 186, 187, 188, 188a, 188b, 189, 189a, 190, 191, 192, 192a, 192b, 193, 193a, 194, 194a, 194b, 195, 195a, 196, 197, 197a, 197b, 198, 199, 200, 201, 201a, 202, 203, 204, 205, 206, 207, 208, 208a, 208b, 209, 210, 210a, 210b, 211, 212, 213, 214, 215, 215a, 215b, 216, 216a, 216b, 216c, 216d, 217, 217a, 217b, 218, 218a, 218b, 219, 220, 221, 221a, 221b, 221c, 221d, 222, 223, 224, 225, 226, 226a, 227, 227a, 228, 228a, 228b, 229, 230, 231, 231a, 231b, 232, 232a, 233, and 233a as depicted in Table C1, or a pharmaceutically acceptable salt thereof; or

Compound No. 101, 102, 103, 104, 105, 105a, 106, 106a, 107, 108, 109, 110, 111, 112, 113, 114, 115, 116, 117, 118, 119, 120, 121, 122, 122a, 122b, 123, 123a, 123b, 124, 125, 126, 126a, 127, 127a, 128, 129, 130, 130a, 131, 131a, 132, 133, 134, 135, 136, 136a, 137, 138, 139, 140, 141, 141a, 142, 143, 144, 145, 146, 146a, 146b, 147, 148, 149, 150, 151, 151a, 151b, 152, 153, 153a, 153b, 154, 155, 155a, 156, 156a, 156b, 156c, 156d, 157, 158, 159, 159a, 159b, 160, and 161 as depicted in Table C1, or a pharmaceutically acceptable salt thereof; or

Compound No. 101, 102, 103, 104, 105, 105a, 106, 106a, 107, 108, 109, 110, 111, 112, 113, 114, 115, 116, 117, 118, 119, 120, 121, 122, 122a, 122b, 123, 123a, 123b, 124, 125, 126, 126a, 127, 127a, 128, 129, 130, 130a, 131, and 131a as depicted in Table C1, or a pharmaceutically acceptable salt thereof.

Embodiment 37. A pharmaceutical composition comprising a compound of any one of Embodiments 1-36, or a pharmaceutically acceptable salt thereof, and a pharmaceutically

Embodiment 38. A compound of any one of Embodiments 1-36, or a pharmaceutically acceptable salt thereof, or the pharmaceutical composition of Embodiment 37 for use in treatment.

Embodiment 39. A compound of any one of Embodiments 1-36 or a pharmaceutically acceptable salt thereof, or the pharmaceutical composition of Embodiment 37 for use in the treatment of a cancer selected from the group consisting of a hematological cancer, breast cancer, gastrointestinal cancer, brain cancer, and lung cancer.

Embodiment 40. The compound or pharmaceutical composition of Embodiment 39, wherein the hematological cancer is selected from the group consisting of diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), peripheral T-cell lymphoma (PTCL), chronic lymphocytic leukemia (CLL), acute lymphocytic leukemia (ALL), and chronic myeloid leukemia (CML); optionally wherein the hematological cancer is selected from the group consisting of diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), chronic lymphocytic leukemia (CLL), acute lymphocytic leukemia (ALL), and chronic myeloid leukemia (CML); optionally wherein the hematological cancer is selected from the group consisting of DLBCL, FL, MCL, BL, PTCL, and ALL (e.g., B-ALL); optionally wherein the hematological cancer is FL or DLBCL; optionally wherein in the hematological cancer is DLBCL.

Embodiment 41. The compound or pharmaceutical composition of Embodiment 38 and an additional therapeutic agent or therapy selected from the group consisting of a PI3K inhibitor, an Abl inhibitor (e.g., a BCR-Abl inhibitor), a BTK inhibitor, a JAK inhibitor, a BRaf inhibitor, a MEK inhibitor, a BCL-2 inhibitor, a Bcl-XL inhibitor, an XPO1 inhibitor, an inhibitor of the polycomb repressive complex 2 (PRC2), an immunomodulatory imide drug, anti-CD19 therapy, anti-CD20 therapy, anti-CD3 therapy, chemotherapy, and a combination thereof for use in treatment; or
an additional therapeutic agent or therapy selected from the group consisting of a PI3K inhibitor, a BTK inhibitor, a JAK inhibitor, a BRaf inhibitor, a MEK inhibitor, a BCL-2 inhibitor, a BCL-XL inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof for use in treatment; or
an additional therapeutic agent or therapy selected from the group consisting of a PI3K inhibitor, a BTK inhibitor, a JAK inhibitor, a BRaf inhibitor, a MEK inhibitor, a Bcl-2 inhibitor, a Bcl-xL inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof for use in treatment; or
an additional therapeutic agent or therapy selected from the group consisting of a PI3K inhibitor, a BTK inhibitor, a JAK inhibitor, a BRaf inhibitor, a MEK inhibitor, a Bcl-2 inhibitor, a Bcl-xL inhibitor, an XPO1 inhibitor, an immunomodulatory imide drug, anti-CD19 therapy, anti-CD20 therapy, anti-CD3 therapy, and a combination thereof for use in treatment.

Embodiment 42. The compound or pharmaceutical composition of Embodiment 39 and an additional therapeutic agent or therapy selected from the group consisting of a PI3K inhibitor, an Abl inhibitor (e.g., a BCR-Abl inhibitor), a BTK inhibitor, a JAK inhibitor, a BRaf inhibitor, a MEK inhibitor, a BCL-2 inhibitor, a Bcl-XL inhibitor, an XPO1 inhibitor, an inhibitor of the polycomb repressive complex 2 (PRC2), an immunomodulatory imide drug, anti-CD19 therapy, anti-CD20 therapy, anti-CD3 therapy, chemotherapy, and a combination thereof; or
an additional therapeutic agent or therapy selected from the group consisting of a PI3K inhibitor, a BTK inhibitor, a JAK inhibitor, a BRaf inhibitor, a MEK inhibitor, a BCL-2 inhibitor, a BCL-XL inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof; or
an additional therapeutic agent or therapy selected from the group consisting of a PI3K a Bcl-xL inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof, or
an additional therapeutic agent or therapy selected from the group consisting of a PI3K inhibitor, a BTK inhibitor, a JAK inhibitor, a BRaf inhibitor, a MEK inhibitor, a Bcl-2 inhibitor, a Bcl-xL inhibitor, an XPO1 inhibitor, an immunomodulatory imide drug, anti-CD19 therapy, anti-CD20 therapy, anti-CD3 therapy, and a combination thereof.

Embodiment 43. A compound of any one of Embodiments 1-36, or a pharmaceutically acceptable salt thereof, or the pharmaceutical composition of Embodiment 37 for use in a method of treating cancer selected from the group consisting of diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), peripheral T-cell lymphoma (PTCL), chronic lymphocytic leukemia (CLL), acute lymphocytic leukemia (ALL), and chronic myeloid leukemia (CML); optionally wherein the cancer is selected from the group consisting of diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), chronic lymphocytic leukemia (CLL), acute lymphocytic leukemia (ALL), and chronic myeloid leukemia (CML); optionally wherein the cancer is selected from the group consisting of DLBCL, FL, MCL, BL, PTCL, and ALL (e.g., B-ALL); optionally wherein the cancer is FL or DLBCL; optionally wherein in the cancer is DLBCL, the method comprising administering to a subject a therapeutically effective amount of a compound of Embodiments 1-36, or a pharmaceutically acceptable salt thereof, or the pharmaceutical composition of Embodiment 37.

Embodiment 44. The compound or pharmaceutical composition of Embodiment 43, wherein the method comprises administering to the subject a therapeutically effective amount of an additional therapeutic agent or therapy selected from the group consisting of a PI3K inhibitor, an Abl inhibitor (e.g., a BCR-Abl inhibitor), a BTK inhibitor, a JAK inhibitor, a BRaf inhibitor, a MEK inhibitor, a BCL-2 inhibitor, a Bcl-XL inhibitor, an XPO1 inhibitor, an inhibitor of the polycomb repressive complex 2 (PRC2), an immunomodulatory imide drug, anti-CD19 therapy, anti-CD20 therapy, anti-CD3 therapy, chemotherapy, and a combination thereof; or
an additional therapeutic agent or therapy selected from the group consisting of a PI3K inhibitor, a BTK inhibitor, a JAK inhibitor, a BRaf inhibitor, a MEK inhibitor, a BCL-2 inhibitor, a BCL-XL inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof; or
an additional therapeutic agent or therapy selected from the group consisting of a PI3K inhibitor, a BTK inhibitor, a JAK inhibitor, a BRaf inhibitor, a MEK inhibitor, a Bcl-2 inhibitor, a Bcl-xL inhibitor, an XPO1 inhibitor, an inhibitor of the polycomb repressive complex 2, an immunomodulatory imide drug, anti-CD19 therapy, anti-CD20 therapy, anti-CD3 therapy, and a combination thereof; or
an additional therapeutic agent or therapy selected from the group consisting of a PI3K inhibitor, a BTK inhibitor, a JAK inhibitor, a BRaf inhibitor, a MEK inhibitor, a Bcl-2 inhibitor, a Bcl-xL inhibitor, an XPO1 inhibitor, an immunomodulatory imide drug, anti-CD19 therapy, anti-CD20 therapy, anti-CD3 therapy, and a combination thereof.

Embodiment 45. The compound or pharmaceutical composition of Embodiment 46, wherein the compound of any one of Embodiments 1-36, or a pharmaceutically acceptable salt thereof, or the pharmaceutical composition of Embodiment 37, and the additional therapeutic agent or therapy are administered simultaneously, separately, or sequentially.

1.-67. (canceled)

68. A compound of Formula (I): 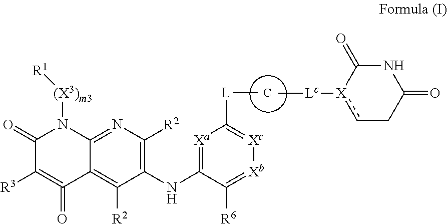
or a pharmaceutically acceptable salt thereof, wherein:
R¹ is selected from the group consisting of: H, halo, cyano, and R^{b1};
m3 is 0, 1, 2, or 3;
each X³ is independently selected from the group consisting of: —O—, —NR^{f}—, —C(═O)—, and C₁₋₃ alkylene optionally substituted with 1-3 R^{c};
provided that the N—(X³)ₘ₃—R¹ moiety does not contain any O—O, N—O, N—N, O-halo, or N-halo bonds;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e},
R³ is selected from the group consisting of:
-A¹-C(R⁴R⁴)-A² and —CH—CH-A², wherein:
A¹ is —O— or —S—;
each R⁴ is independently selected from the group consisting of: H, C₁₋₆ alkyl, and C₁₋₆ haloalkyl; or
the pair of R⁴ taken together with the carbon atom to which each is attached form a C₃₋₆ cycloalkyl ring or a 4-8 membered heterocyclyl ring, wherein the C₃₋₆ cycloalkyl ring or 4-8 membered heterocyclyl ring is optionally substituted with 1-3 R^{g};
A² is selected from the group consisting of: —C(O)OH, —C(O)NH₂, —C(O)R³⁴, —C(O)OR^{3A}, —C(O)NR^{3A}R^{f}, —S(O)₁₋₂ (C₁₋₆ alkyl), —P(O)—(C₁₋₆ alkyl)₂, and —C(═NH)NH₂, wherein:
R^{3A} is selected from the group consisting of: C₁₋₆ alkyl, C₃₋₆ alkenyl, C₃₋₆ alkynyl, C₃₋₆ cycloalkyl, and 3-8 membered heterocyclyl, each of which is optionally substituted with 1-6 substituents independently selected from the group consisting of R^{a} and —(C₀₋₃ alkylene)-R^{b1};
X^{a} and X^{c} are independently selected from the group consisting of: N, CH, and CF, provided that one or both of X^{a} and X^{c} is N;
X^{b} is selected from the group consisting of N and CR^{x1};
R⁶ and R^{x1} are each independently selected from the group consisting of: H, halo, C₁₋₂ alkyl, C₁₋₂ haloalkyl, C₁₋₂ alkoxy, CN, and —C═CH;
L is -(L^{A})ₙ₁-, wherein
n1 is an integer from 1 to 5; and
each L^{A} is independently selected from the group consisting of: L^{A1}, L^{A3}, and L^{A4}, provided that 1-3 occurrences of L^{A} is L^{A4};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, O—O, N—N, N—S(O) o, or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, S(O)₀₋₂ (C₁₋₆ alkyl), S(O)₀₋₂ (C₁₋₆ haloalkyl), S(O)₁₋₂N(R^{f})₂, —R^{b}, and C₁₋₆ alkyl optionally substituted with 1-6 R^{c};
Ring C is selected from the group consisting of: 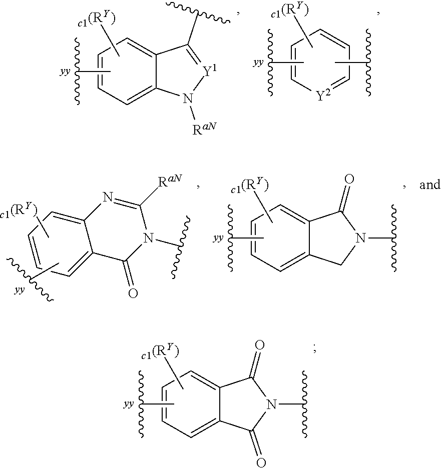
c1 is 0, 1, 2, or 3;
each R^{Y} is independently selected from the group consisting of: R^{a} and R^{b};
R^{aN} is H or C₁₋₆ alkyl optionally substituted with 1-3 R^{c};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —C(═O)—, —N(R^{d})—, and O, provided that when X is N, then L^{C} is other than O; and
further provided that when Ring C is attached to -L^{C}- via a ring nitrogen, then X is CH, and L^{C} is a bond;
each R^{a} is independently selected from the group consisting of:
(a) halo;
(b) cyano;
(c) —OH;
(d) oxo;
(e) C₁₋₆ alkoxy optionally substituted with 1-6 R^{c};
(f) —NR^{d}R^{e},
(g) C(═O) C₁₋₆ alkyl optionally substituted with 1-6 R^{c};
(h) C(═O)OH;
(i) C(═O)OC₁₋₆ alkyl;
(j) C(═O)OC₁₋₆ haloalkyl;
(k) C(—═O)N(R^{f})₂;
(l) S(O)₀₋₂ (C₁₋₆ alkyl);
(m) S(O)₀₋₂ (C₁₋₆ haloalkyl);
(n) S(O)₁₋₂N(R^{f})₂; and
(o) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂ (C₁₋₆ alkyl), S(O)₀₋₂ (C₁₋₆ haloalkyl), and S(O)₁₋₂N(R^{f})₂;
each R^{d} and R^{e} is independently selected from the group consisting of: H, C(═O) C₁₋₆ alkyl, C(═O) C₁₋₆ haloalkyl, C(═O)OC₁₋₆ alkyl, C(═O)OC₁₋₆ haloalkyl, C(═O)N(R^{f})₂, S(O)₁₋₂ (C₁₋₆ alkyl), S(O)₁₋₂ (C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, cyano, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

69. The compound of claim 68, wherein R³ is -A¹-C(R⁴R⁴)-A²; A¹ is O; each R⁴ is H; and A² is —C(O)NH₂ or —C(O)NR^{3A}R^{f}, wherein R^{3A} is C₁₋₃ alkyl optionally substituted with 1-6 R^{c}.

70. The compound of claim 68, wherein R³ is

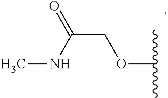 [SMILES: *OCC(=O)NC]

71. The compound of claim 68, wherein R⁶ is —C₁ or —F; and each R² is H.

72. The compound of claim 68, wherein m3 is 1; X³ is C₁₋₃ alkylene; and R¹ is —H.

73. The compound of claim 68, wherein R³ is -A¹-C(R⁴R⁴)-A², wherein A¹ is O;
each R⁴ is H; and A² is —C(O)NH₂ or —C(O)NR^{3A}R^{f}, wherein R^{3A} is C₁₋₃ alkyl optionally substituted with 1-6 R^{c};
each R² is H;
X^{a} is N or CH;
X^{c} is N;
X^{b} is CH; and
R⁶ is —F or —Cl.

74. The compound of claim 68, wherein Ring C is

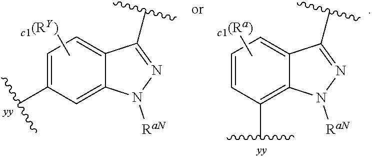

75. The compound of claim 74, wherein cl is 0 or 1; and R^{Y} is halo.

76. The compound of claim 74, wherein X is CH; and L^{C} is a bond.

77. The compound of claim 68, wherein L is selected from the group consisting of: 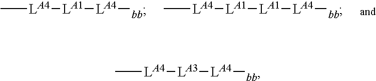
wherein bb represents the point of attachment to Ring C.

78. The compound of claim 77, wherein each L^{A4} is independently a monocyclic 4-6 membered nitrogen-containing heterocyclylene optionally substituted with 1-3 R^{a}.

79. The compound of claim 77, wherein one L^{A4} is a monocyclic 4-6 membered monocyclic nitrogen-containing heterocyclylene optionally substituted with 1-3 Rª; and the other L^{A4} is a bicyclic 6-12 membered nitrogen-containing heterocyclylene optionally substituted with 1-3 Rª.

80. The compound of claim 77, wherein each R^{a} present on L^{A4} is independently selected from the group consisting of: —F, CN, C₁₋₃ alkoxy, OH, and C₁₋₃ alkyl optionally substituted with 1-3 F.

81. The compound of claim 68, wherein the compound is a compound of Formula (I-a-2): 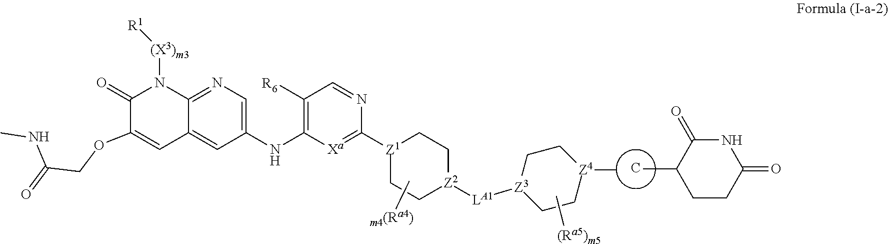
or a pharmaceutically acceptable salt thereof, wherein:
X^{a} is N or CH;
R⁶ is —F or —Cl;
m3 is 1;
X³ is C₁₋₃ alkylene;
R¹ is H;
L^{A1} is CH₂, CHMe, or CMe₂;
Z¹ and Z² are independently selected from the group consisting of: CH, CR¹⁴, and N;
Z³ and Z⁴ are independently selected from the group consisting of: CH, CR^{a5}, and N, provided that at least one of Z¹ and Z² is N; at least one of Z³ and Z⁴ is N; and when Z² is N, then Z³ is CH or CR^{a5};
m4 and m5 are independently selected from the group consisting of: 0, 1, and 2; and
each R^{a4} and R^{a5} is independently selected from the group consisting of: —F, CN, C₁₋₃ alkoxy, OH, and C₁₋₃ alkyl optionally substituted with 1-3 F; and
Ring C is selected from the group consisting of: 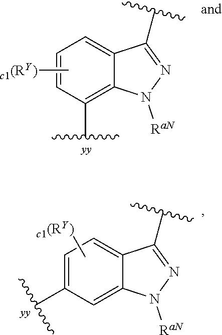
wherein:
c1 is 0 or 1,
R^{Y} is selected from the group consisting of halo and C₁₋₃ alkyl optionally substituted with 1-3 F, and
R^{aN} is C₁₋₃ alkyl.

82. The compound of claim 81, wherein Z¹ is N; Z² is CH or CR^{a4}; and Z³ is N.

83. The compound of claim 81, wherein Z¹ is N; Z² is CH; Z³ is N; Z⁴ is CH; one of m4 and m5 is 0 or 1; and the other of m4 and m5 is 1.

84. The compound of claim 68, wherein the compound is a compound of Formula (I-b-1): 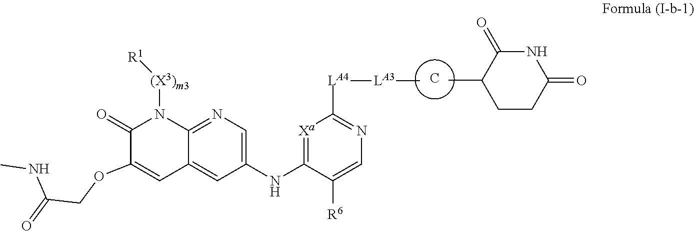
or pharmaceutically acceptable salts thereof, wherein:
X^{a} is N or CH;
R⁶ is —F or —Cl;
m3 is 1;
X³ is C₁₋₃ alkylene;
R¹ is H;
L^{A4} is a 6-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F; and
L^{A3} is —NH—, —N(C₁₋₃ alkyl)-, or —O—; and
Ring C is selected from the group consisting of: 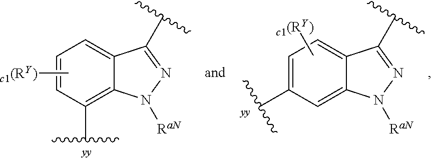
wherein:
c1 is 0 or 1,
R^{Y} is selected from the group consisting of halo and C₁₋₃ alkyl optionally substituted with 1-3 F, and
R^{aN} is C₁₋₃ alkyl.

85. The compound of claim 84, wherein L^{A4} is a bicyclic spirocyclic 6-12 membered nitrogen-containing heterocyclylene optionally substituted with 1-3 R^{a}, wherein L^{A4} contains 1-2 ring nitrogen atoms and no additional ring heteroatoms.

86. A pharmaceutical composition comprising a compound of claim 68, or a pharmaceutically acceptable salt thereof, and a pharmaceutically acceptable excipient.

87. A method for treating a cancer in a subject in need thereof, the method comprising administering to the subject a therapeutically effective amount of a compound of claim 68, or a pharmaceutically acceptable salt thereof.


SEQUENCE LISTING (1 sequence)

SEQ ID NO: 1 - protein, 751 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHRKCNYSFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP